(19) United States
(12) Patent Application Publication
(10) Pub. No.: US 2023/0002398 A1
(43) Pub. Date: Jan. 5, 2023

(54) HETEROCYCLIC COMPOUND AND LIGHT-EMITTING DEVICE INCLUDING THE SAME

(71) Applicant: Samsung Electronics Co., Ltd., Suwon-si (KR)

(72) Inventors: Soonok Jeon, Suwon-si (KR); Inkoo KIM, Suwon-si (KR); Sangmo KIM, Hwaseong-si (KR); Juhyun KIM, Seoul (KR); Yusuke MARUYAMA, Hwaseong-si (KR); Won-joon SON, Suwon-si (KR); Dmitry ANDROSOV, Hwaseong-si (KR); Hyeonho Choi, Seoul (KR)

(21) Appl. No.: 17/318,939

(22) Filed: May 12, 2021

(30) Foreign Application Priority Data
May 12, 2020 (KR) 10-2020-0056667
May 11, 2021 (KR) 10-2021-0060729

(51) Int. Cl. C07D 487/22; H01L 51/00; C07D 487/16; C07F 5/02

(52) U.S. Cl. CPC C07D 487/22; H01L 51/0072; H01L 51/5024; C07F 5/02; H01L 51/006; C07D 487/16

(57) ABSTRACT

Disclosed are a heterocyclic compound represented by Formula 1-1 or 1-2,

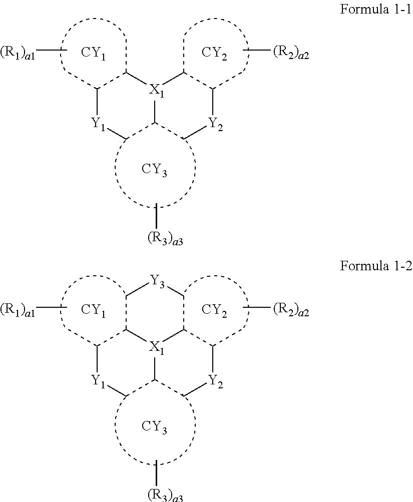

wherein, in Formulae 1-1 and 1-2, X₁, CY₁ to CY₃, R₁ to R₃, and a1 to a3, are the same as described in the specification. Also disclosed is an organic light-emitting device including the same.

### CROSS-REFERENCE TO RELATED APPLICATION

This application is based on and claims priority to Korean Patent Application Nos. 10-2020-0056667, filed on May 12, 2020 and 10-2021-0060729, filed on May 11, 2021 in the Korean Intellectual Property Office, and all the benefits accruing therefrom under 35 U.S.C. § 119, the entire content of which is incorporated by reference herein.

### BACKGROUND

### 1. Field

One or more embodiments relate to a heterocyclic compound having a novel structure and an organic light-emitting device including the same.

### 2. Description of the Related Art

Organic light-emitting devices (OLEDs) are self-emissive devices that produce full-color images, and also have wide viewing angles, high contrast ratios, short response times, and excellent characteristics in terms of luminance, driving voltage, and response speed, compared to devices in the art.

In an example, an organic light-emitting device includes an anode, a cathode, and an organic layer between the anode and the cathode, wherein the organic layer includes an emission layer. A hole transport region may be located between the anode and the emission layer, and an electron transport region may be located between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transition from an excited state to a ground state to thereby generate light.

Various types of organic light emitting devices are known. However, there still remains a need in OLEDs having one or more, preferably all, of low voltage, high efficiency, high color purity, and long lifetime characteristics

### SUMMARY

Provided are a heterocyclic compound having a novel structure and an organic light-emitting device including the same, wherein the organic light-emitting device has low voltage, high efficiency, high color purity, and long lifetime characteristics.

Additional aspects will be set forth in part in the description, which follows and, in part, will be apparent from the description, or may be learned by practice of the presented embodiments of the disclosure.

An aspect of the present disclosure provides a heterocyclic compound represented by Formula 1-1 or 1-2:

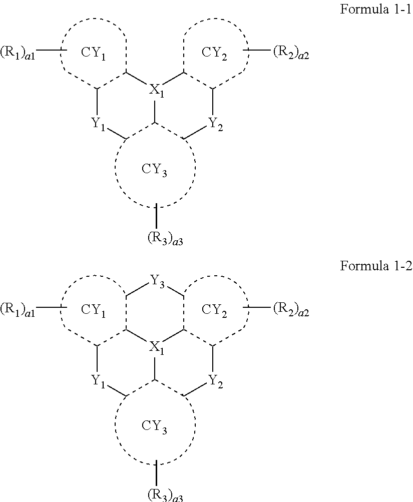

wherein, in Formulae 1-1 and 1-2,

X₁ may be B or N,

ring CY₁, ring CY₂, and ring CY₃ may each independently be a C₅-C₃₀ carbocyclic group or a C₁-C₃₀ heterocyclic group,

at least one of ring CY₁, ring CY₂, and ring CY₃ may include a pyrimidine group,

Y₁, Y₂, and Y₃ may each independently be O, S, Se, B(R₄), N(R₄), C(R₄)(R₅), Si(R₄)(R₅), Ge(R₄)(R₅), P(R₄), P(═O)(R₄), P(═S)(R₄), or Al(R₄),

R₁, R₂, R₃, R₄, and R₅ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

two neighboring groups from among R₁ to R₅ may be optionally bonded to each other to form a C₅-C₃₀ carbocyclic group unsubstituted or substituted with R₁₀ or a C₁-C₃₀ heterocyclic group unsubstituted or substituted with R₁₀, and R₁₀ is the same as described in connection with R₁,

a1 to a3 may each independently be an integer from 1 to 10,

any one of the following condition (i) to (iii) is satisfied,

(i) when any one of ring CY₁, ring CY₂, and ring CY₃ contains pyrimidine, at least one of Y₁ to Y₃ is N(R₄),

(ii) when two of ring CY₁, ring CY₂, and ring CY₃ each contain pyrimidine, at least one of Y₁ to Y₃ is N(R₄),

(iii) when each of ring CY₁, ring CY₂, and ring CY₃ contains pyrimidine, Y₁ to Y₃ may each independently be Se, B(R₄), N(R₄), C(R₄)(R₅), Si(R₄)(R₅), Ge(R₄)(R₅), P(R₄), P(═O)(R₄), P(═S)(R₄), or Al(R₄),

at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ arylalkyl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, or the substituted monovalent non-aromatic condensed heteropolycyclic group may be

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkylthio group, or a C₁-C₆₀ alkoxy group,

a C₁-C₆₀ alkyl group, a C₂-C₆₀ alkenyl group, a C₂-C₆₀ alkynyl group, a C₁-C₆₀ alkylthio group, or a C₁-C₆₀ alkoxy group, each independently substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or —P(═O)(Q₁₈)(Q₁₉),

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group,

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group, each independently substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅)-B(Q₂₆)(Q₂₇), or —P(═O)(Q₂₈)(Q₂₉), or

—Si(Q₃₁)(Q₃₂)(Q₃₃), —N(Q₃₄)(Q₃₅), —B(Q₃₆)(Q₃₇), or —P(═O)(Q₃₈)(Q₃₉),

wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, or a substituted or unsubstituted monovalent non-aromatic condensed heteropolycyclic group.

Another aspect provides an organic light-emitting device including a first electrode, a second electrode, and an organic layer including an emission layer between the first electrode and the second electrode, wherein the organic layer includes the heterocyclic compound represented by Formula 1-1 or 1-2.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic cross-sectional view of an organic light-emitting device according to an embodiment;

FIGS. 2A, 2B, and 2C each show a diagram schematically illustrating energy transfer in an emission layer of an organic light-emitting device according to an embodiment.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list, for example, “at least one of a, b, or c” indicates only a, only b, only c, both a and b, both a and c, both b and c, all of a, b, and c, or variations thereof.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10%, 5% of the stated value.

In any formula, * and *′ each indicate a binding site to a neighboring atom or a neighboring functional group.

An aspect of the present disclosure provides a heterocyclic compound represented by Formula 1-1 or 1-2 below:

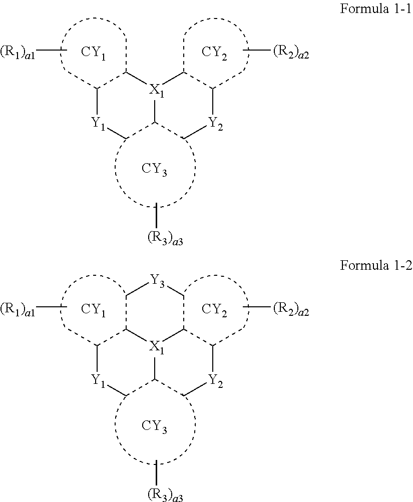

Formulae 1-1 and 1-2 will be described in detail.

X₁ in Formulae 1-1 and 1-2 may be B or N. For example, X₁ may be B.

Ring CY₁, ring CY₂, and ring CY₃ in Formulae 1-1 and 1-2 may each independently be a C₅-C₃₀ carbocyclic group or a C₁-C₃₀ heterocyclic group, and at least one of ring CY₁, ring CY₂, and ring CY₃ may include a pyrimidine group.

In an embodiment, ring CY₁, ring CY₂, and ring CY₃ may each independently be group A, group B, a condensed ring in which two or more groups of group A are condensed with each other, a condensed ring in which two or more groups of group B are condensed with each other, or a condensed ring in which at least one group A is condensed with at least one group B.

Group A may be a cyclopenta-1,3-diene group, an indene group, an azulene group, a benzene group, a naphthalene group, an anthracene group, a phenanthrene group, a tetracene group, a tetraphene group, a pyrene group, a chrysene group, a triphenylene group, or a fluorene group, or

Group B may be a furan group, a thiophene group, a pyrrole group, borole group, a silole group, a pyrrolidine group, an imidazole group, a triazole group, an oxazole group, an isoxazole group, a thiazole group, an isothiazole group, a pyridine group, a pyrimidine group, a pyridazine group, a triazine group, an indole group, an isoindole group, an indolizine group, a quinoline group, an isoquinoline group, a quinoxaline group, an isoquinoxaline group, a carbazole group, a dibenzofuran group, a dibenzothiophene group, a dibenzosilole group, or a dibenzoborole group.

For example, ring CY₁, ring CY₂, and ring CY₃ may each independently be a benzene group, a naphthalene group, a fluorene group, a pyridine group, a pyrimidine group, a pyrazine group, a quinoline group, an isoquinoline group, a quinazoline group, a quinoxaline group, a phthalazine group, a carbazole group, an azacarbazole group, a dibenzofuran group, a dibenzothiophene group, a dibenzoselenophene group, a dibenzosilole group, an azadibenzofuran group, an azadibenzothiophene group, an azadibenzoselenophene group, or an azadibenzosilole group, but embodiments of the present disclosure are not limited thereto.

In an embodiment, when 1 or 2 rings of the ring CY₁ to ring CY₃ include a pyrimidine group, the remaining ring may be a benzene group, a naphthalene group, or a fluorene group.

For example, ring CY₃ may be a pyrimidine group and ring CY₁ and ring CY₂ may each be a benzene group; ring CY₁ and ring CY₂ may each be a pyrimidine group and ring CY₃ may be a benzene group; or ring CY₁, ring CY₂, and ring CY₃ may each be a pyrimidine group, but embodiments of the present disclosure are not limited thereto.

Y₁, Y₂, and Y₃ in Formulae 1-1 and 1-2 may each independently be O, S, Se, B(R₄), N(R₄), C(R₄)(R₅), Si(R₄)(R₅), Ge(R₄)(R₅), P(R₄), P(═O)(R₄), P(═S)(R₄), or Al(R₄).

In an embodiment, at least one of Y₁ to Y₃ may be N(R₄). For example, each of Y₁ to Y₃ may be N(R₄), but embodiments of the present disclosure are not limited thereto.

In an embodiment, X₁ may be B and each of Y₁ and Y₂ may be N(R₄).

In an embodiment, X₁ may be B and Y₁ to Y₃ may be identical to each other. For example, X₁ may be B and Y₁ to Y₃ may each be C(R₄)(R₅) or N(R₄).

R₁, R₂, R₃, R₄ and R₅ in Formulae 1-1 and 1-2 may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q), and neighboring two groups of R₁ to R₅ may optionally be bonded to form a C₅-C₃₀ carbocyclic group unsubstituted or substituted with R₁₀ or a C₁-C₃₀ heterocyclic group unsubstituted or substituted with R₁₀. In this regard, R₁₀ may be the same as described in connection with R₁.

In an embodiment, R₁ to R₅ may each independently be:

hydrogen, deuterium, —F, —Cl, —Br, —I, a hydroxyl group, a cyano group (CN), a nitro group, an amino group, a C₁-C₆₀ alkyl group, a C₁-C₆₀ alkylthio group, or a C₁-C₆₀ alkoxy group;

a C₁-C₆₀ alkyl group, a C₁-C₆₀ alkylthio group, or a C₁-C₆₀ alkoxy group, each independently substituted with at least one of deuterium, —F, —Cl, —Br, —I, a hydroxyl group, a cyano group (CN), a nitro group, an amino group, a phenyl group, a biphenyl group, a naphthyl group, a fluorenyl group, a phenanthrenyl group, an anthracenyl group, a fluoranthenyl group, a triphenylenyl group, a pyrenyl group, or a chrysenyl group;

a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group;

a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each independently substituted with at least one of deuterium, —F, —Cl, —Br, —I, a hydroxyl group, a cyano group (CN), a nitro group, an amino group, a C₁-C₆₀ alkyl group, a C₁-C₆₀ alkoxy group, a C₁-C₆₀ alkylthio group, a C₇-C₆₀ arylalkyl group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a carbazolyl group, —Si(Q₃₁)(Q₃₂)(Q₃₃), —N(Q₃₄)(Q₃₅), —B(Q₃₆)(Q₃₇), or —P(═O)(Q₃₈)(Q₃₉); or

—N(Q₁)(Q₂), —Si(Q₃)(Q₄)(Q₅), —B(Q₆)(Q₇), or —P(═O)(Q₈)(Q₉),

but embodiments of the present disclosure are not limited thereto.

For example, R₁ to R₃ may each independently be:

hydrogen, deuterium, —F, —Cl, —Br, —I, a hydroxyl group, a cyano group, a nitro group, a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group; or

a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group, each independently substituted with at least one of deuterium, —F, —Cl, —Br, —I, a hydroxyl group, a cyano group, a nitro group, or a phenyl group.

For example, R₄ and R₅ may each independently be:

hydrogen, deuterium, —F, —Cl, —Br, —I, a hydroxyl group, a cyano group (CN), a nitro group, an amino group, a C₁-C₆₀ alkyl group, a C₁-C₆₀ alkylthio group, or a C₁-C₆₀ alkoxy group;

a C₁-C₆₀ alkyl group, a C₁-C₆₀ alkylthio group, or a C₁-C₆₀ alkoxy group, each independently substituted with at least one of deuterium, —F, —Cl, —Br, —I, a hydroxyl group, a cyano group (CN), a nitro group, an amino group, or a phenyl group; and

a group represented by one of Formulae 4-1 to 4-42, but embodiments of the present disclosure are not limited thereto:

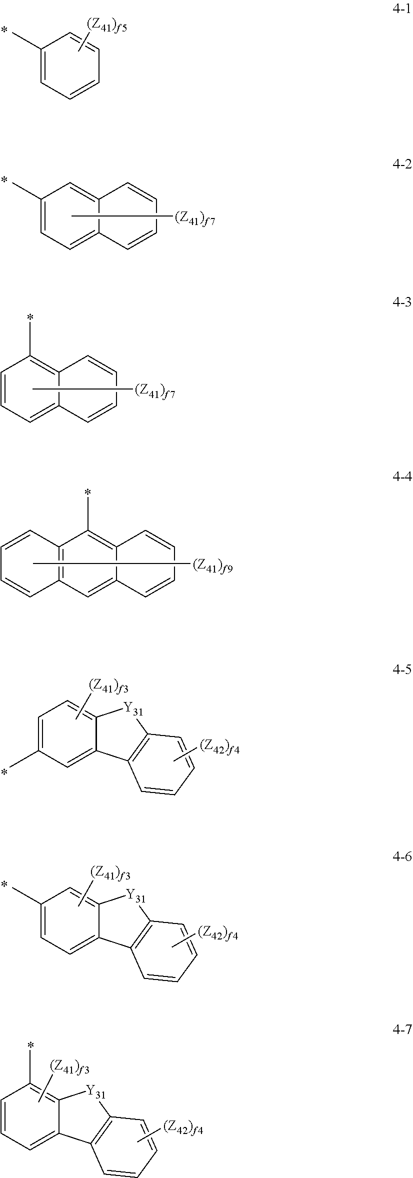

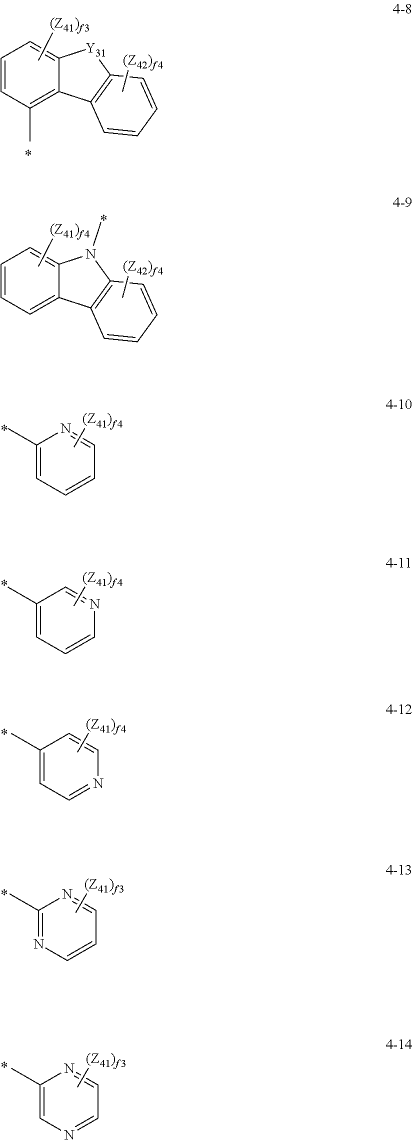

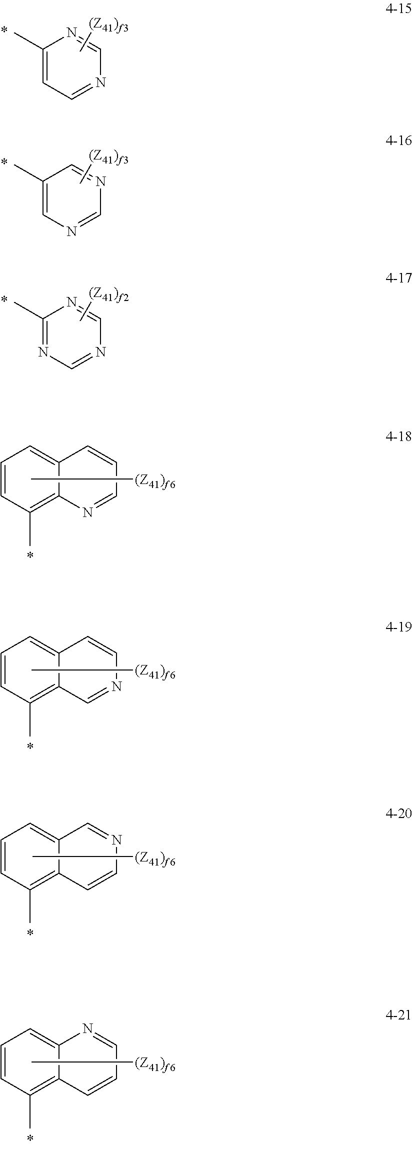

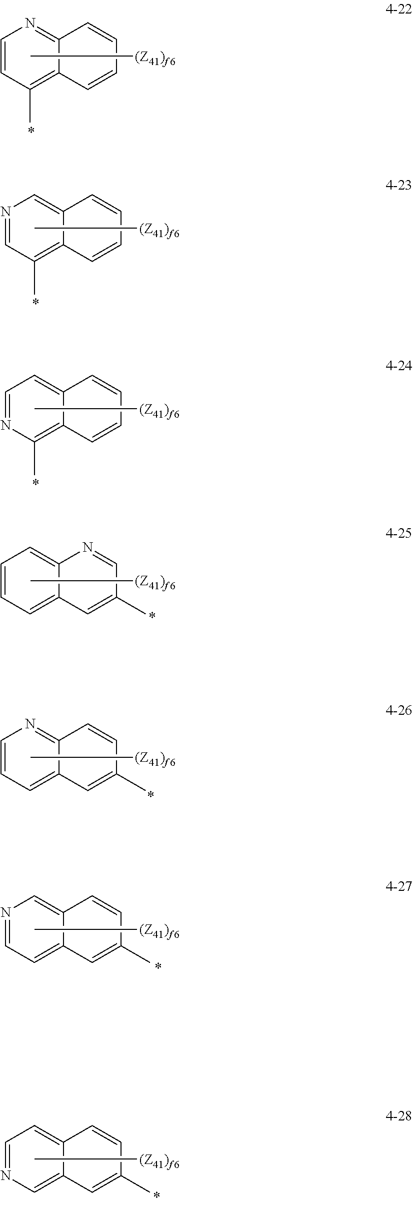

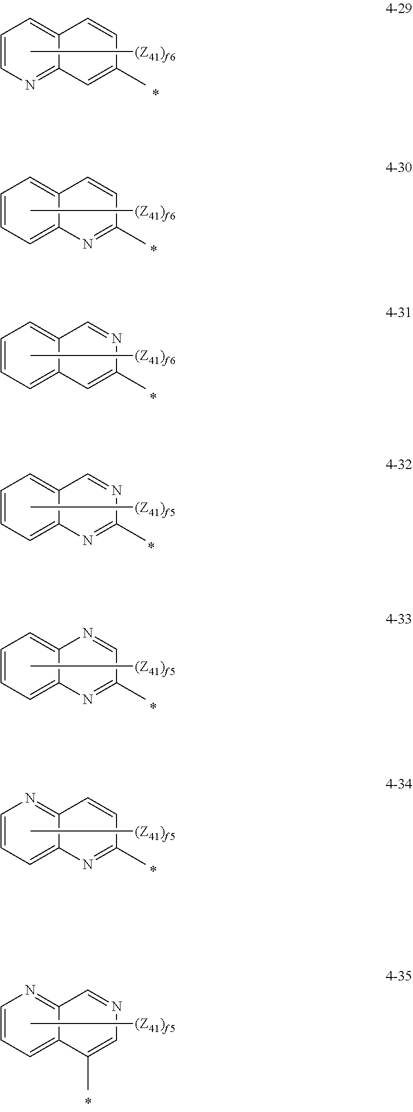

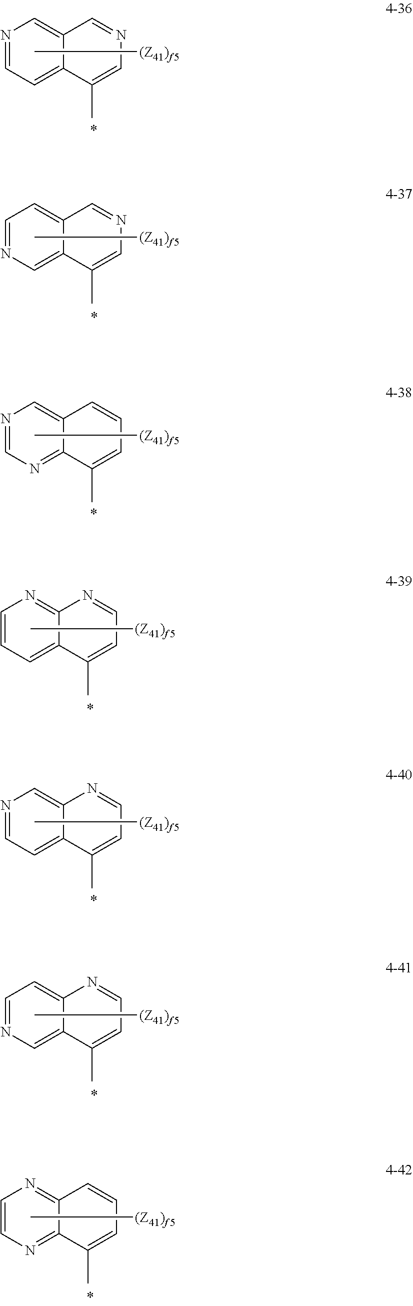

wherein, in Formulae 4-1 to 4-42,

Y₃₁ may be O, S, C(Z₄₃)(Z₄₄), N(Z₄₃), or Si(Z₄₃)(Z₄₄),

Z₄₁ to Z₄₄ may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a C₇-C₆₀ arylalkyl group, a cyclopentyl group, a cyclohexyl group, a cycloheptyl group, a cyclopentenyl group, a cyclohexenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group;

a cyclopentyl group, a cyclohexyl group, a cycloheptyl group, a cyclopentenyl group, a cyclohexenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, or a C₇-C₆₀ arylalkyl group (for example, a cumyl group),

f3 may be an integer from 1 to 3,

f4 may be an integer from 1 to 4,

f5 may be an integer from 1 to 5,

f6 may be an integer from 1 to 6,

f7 may be an integer from 1 to 7,

f9 may be an integer from 1 to 9,

* indicates a binding site to a neighboring atom, and

substituent Z₄₁ is present on each ring through which the bond with the substituent Z₄₁ passes.

For example, R₄ and R₅ may each independently be:

hydrogen, deuterium, —F, —Cl, —Br, —I, a hydroxyl group, a cyano group, a nitro group, a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group; or

a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group, each independently substituted with at least one of deuterium, —F, —Cl, —Br, —I, a hydroxyl group, a cyano group, a nitro group, or a phenyl group; and

a group represented by one of Formulae 4-1 to 4-8, and

Y₃₁ in Formulae 4-5 to 4-8 may be S, C(Z₄₃)(Z₄₄), N(Z₄₃), or Si(Z₄₃)(Z₄₄).

In Formulae 1-1 and 1-2, a1 indicates the number of R₁(s) and may be an integer from 1 to 10, wherein, when a1 is 2 or more, two or more of R₁(s) may be identical to or different from each other; a2 indicates the number of R₂(s) and may be an integer from 1 to 10, wherein, when a2 is 2 or more, two or more of R₂(s) may be identical to or different from each other; and a3 indicates the number of R₃(s) and may be an integer from 1 to 10, wherein, when a3 is 2 or more, two or more of R₃(s) may be identical to or different from each other.

A heterocyclic compound represented by Formula 1-1 or 1-2 may satisfy any of the following conditions (i) to (iii):

(i) when any one of ring CY₁, ring CY₂, and ring CY₃ contains pyrimidine, at least one of Y₁ to Y₃ is N(R₄);

(ii) when two of ring CY₁, ring CY₂, and ring CY₃ each contain pyrimidine, at least one of Y₁ to Y₃ is N(R₄); and

(iii) when each of ring CY₁, ring CY₂, and ring CY₃ contains pyrimidine, Y₁ to Y₃ may each independently be Se, B(R₄), N(R₄), C(R₄)(R₅), Si(R₄)(R₅), Ge(R₄)(R₅), P(R₄), P(═O)(R₄), P(═S)(R₄), or Al(R₄).

In an embodiment, the heterocyclic compound may be represented by one of Formulae 2-1 to 2-25:

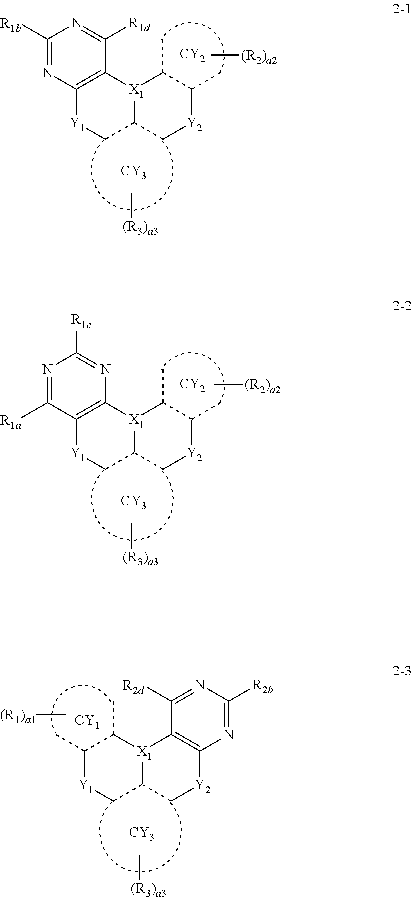

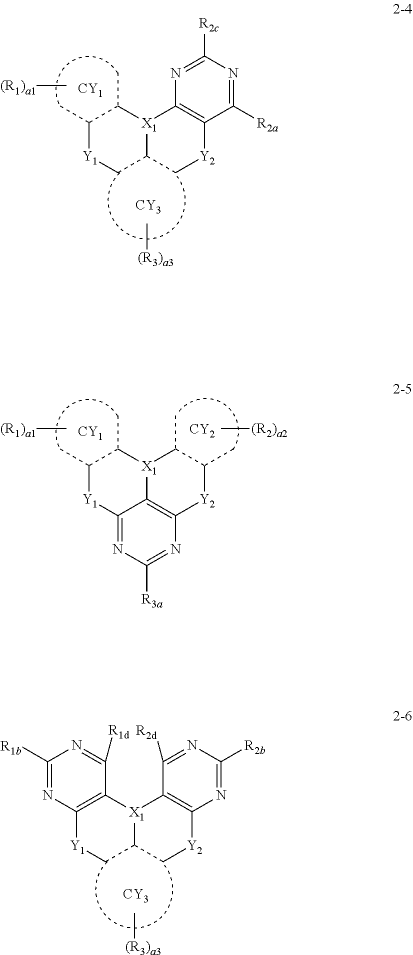

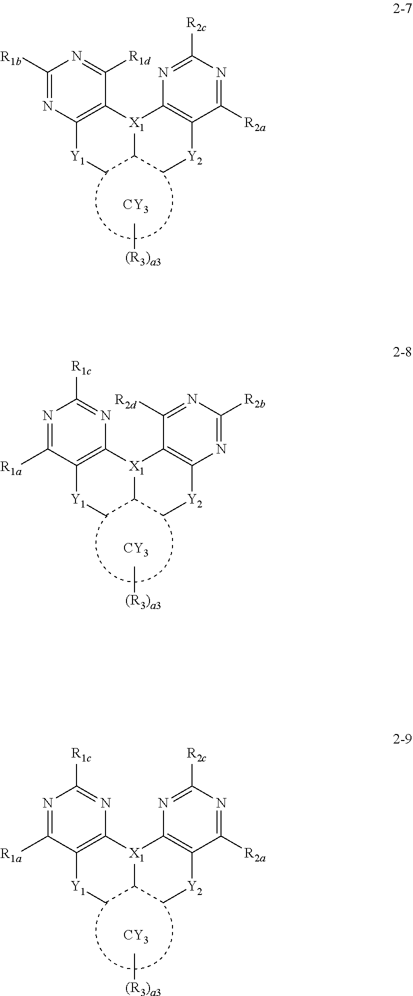

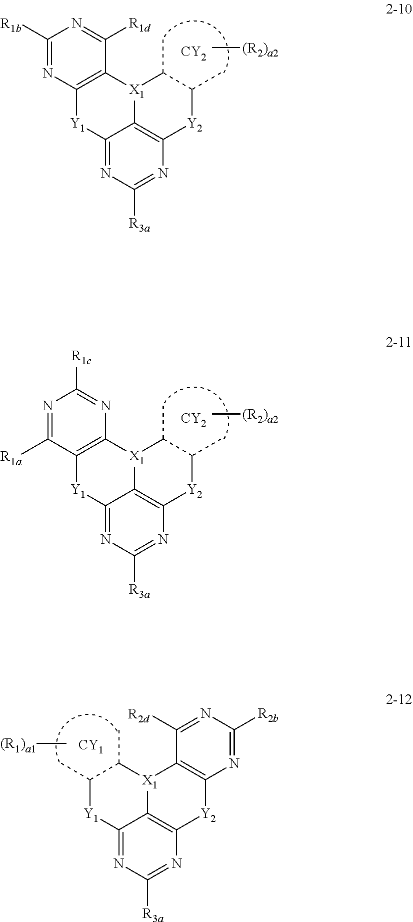

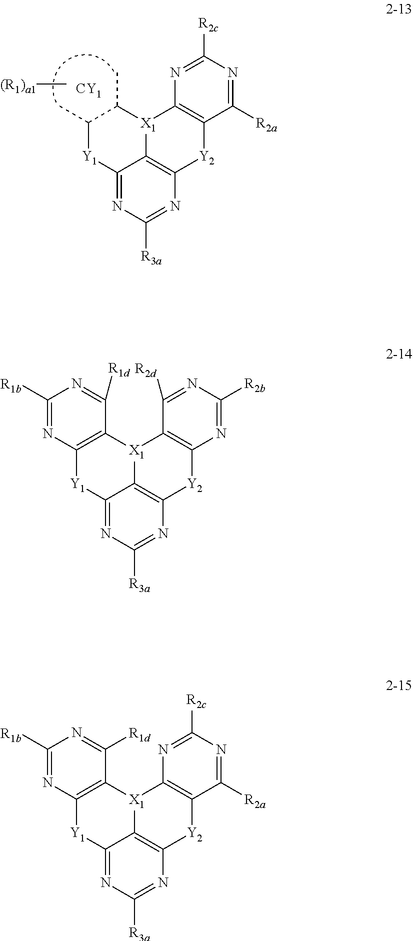

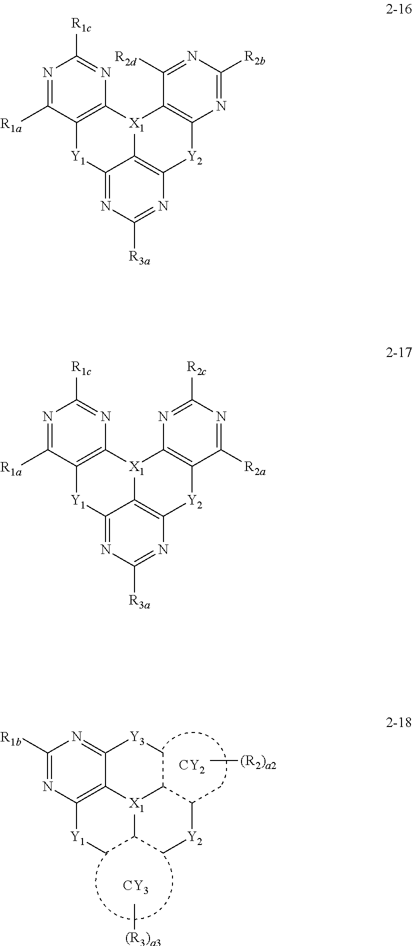

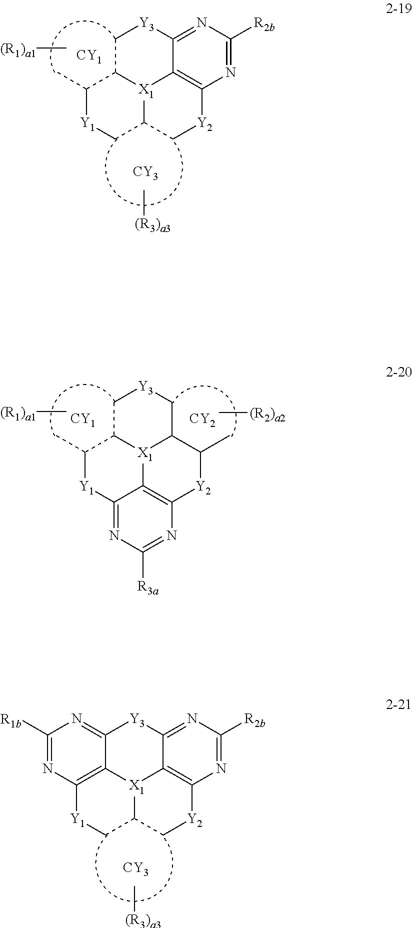

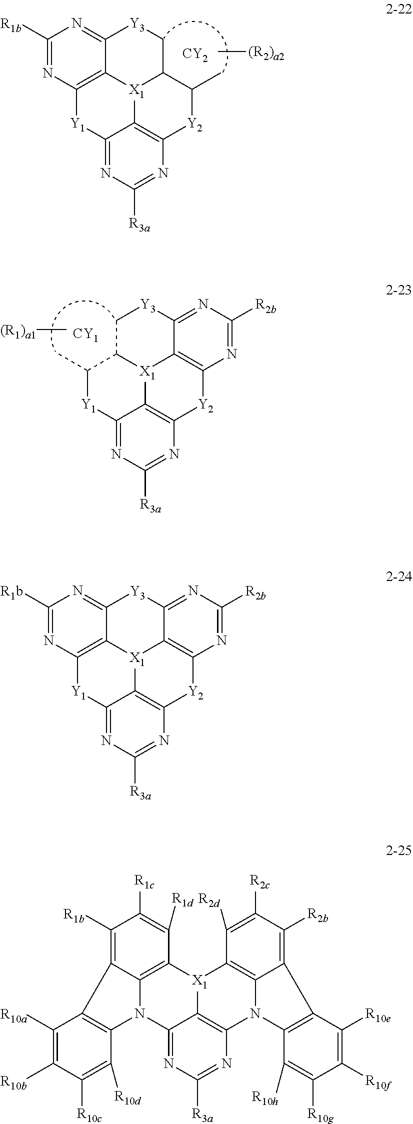

wherein, in Formulae 2-1 to 2-25,

ring CY₁, ring CY₂, ring CY₃, X₁, Y₁, Y₂, Y₃, R₁, R₂, R₃, a1, a2, and a3 are the same as described in the present specification,

R₁ₐ to R_{1d} are each independently the same as described in connection with R₁,

R₂ₐ to R_{2d} are each independently the same as described in connection with R₂,

R₃ₐ is the same as described in connection with R₃, and

R₁₀ₐ to R₁₀ₕ are each independently the same as described in connection with R₁₀.

For example, in Formulae 2-1 to 2-13 and 2-18 to 2-23,

ring CY₁ to ring CY₃ may each independently be a benzene group, a naphthalene group, or a fluorene group.

In an embodiment, the heterocyclic compound may be of Compounds of groups 2-1 to 2-25 below:

### Group 2-1

The compounds are represented by the formula 2-1 below, wherein CY₂ and CY₃ are each a benzene group, X₁ is B, and Y1, Y2, R1b, R1d, (R2)a2, (R3)a3 are each shown in the table 1 below,

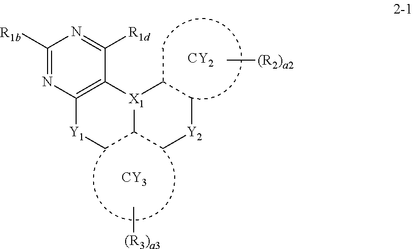

TABLE 1

| Compound No. | Y1 | Y2 | R1b | R1d | (R2)a2 | (R3)a3 |
|---|---|---|---|---|---|---|
| 2-1-001 | N—Ph | N—Ph | H | H | H | H |
| 2-1-002 | N—Ph | N—Ph | t-bu | H | H | H |
| 2-1-003 | N—Ph | N—Ph | methyl | H | H | H |
| 2-1-004 | N—Ph | N—Ph | t-bu | t-bu | t-bu | t-bu |
| 2-1-005 | N—Ph | N—Ph | methyl | methyl | methyl | methyl |
| 2-1-006 | N—Ph | N—Ph | b1 | H | H | H |
| 2-1-007 | N—Ph | N—Ph | b2 | H | H | H |
| 2-1-008 | N—Ph | N—Ph | b3 | H | H | H |
| 2-1-009 | N—Ph | N—Ph | H | H | H | a1 |
| 2-1-010 | N—Ph | N—Ph | t-bu | H | H | a1 |
| 2-1-011 | N—Ph | N—Ph | methyl | H | H | a1 |
| 2-1-012 | N—Ph | N—Ph | b1 | H | H | a1 |
| 2-1-013 | N—Ph | N—Ph | b2 | H | H | a1 |
| 2-1-014 | N—Ph | N—Ph | b3 | H | H | a1 |
| 2-1-015 | N—Ph | N—Ph | H | H | H | a2 |
| 2-1-016 | N—Ph | N—Ph | t-bu | H | H | a2 |
| 2-1-017 | N—Ph | N—Ph | methyl | H | H | a2 |
| 2-1-018 | N—Ph | N—Ph | b1 | H | H | a2 |
| 2-1-019 | N—Ph | N—Ph | b2 | H | H | a2 |
| 2-1-020 | N—Ph | N—Ph | b3 | H | H | a2 |
| 2-1-021 | N—Ph | N—Ph | H | H | H | a3 |
| 2-1-022 | N—Ph | N—Ph | t-bu | H | H | a3 |
| 2-1-023 | N—Ph | N—Ph | methyl | H | H | a3 |
| 2-1-024 | N—Ph | N—Ph | b1 | H | H | a3 |
| 2-1-025 | N—Ph | N—Ph | b2 | H | H | a3 |
| 2-1-026 | N—Ph | N—Ph | b3 | H | H | a3 |
| 2-1-027 | N—Ph | N—Ph | H | H | H | a4 |
| 2-1-028 | N—Ph | N—Ph | t-bu | H | H | a4 |
| 2-1-029 | N—Ph | N—Ph | methyl | H | H | a4 |
| 2-1-030 | N—Ph | N—Ph | b1 | H | H | a4 |
| 2-1-031 | N—Ph | N—Ph | b2 | H | H | a4 |
| 2-1-032 | N—Ph | N—Ph | b3 | H | H | a4 |
| 2-1-033 | N—Ph | N—Ph | H | H | H | a5 |
| 2-1-034 | N—Ph | N—Ph | t-bu | H | H | a5 |
| 2-1-035 | N—Ph | N—Ph | methyl | H | H | a5 |
| 2-1-036 | N—Ph | N—Ph | b1 | H | H | a5 |
| 2-1-037 | N—Ph | N—Ph | b2 | H | H | a5 |
| 2-1-038 | N—Ph | N—Ph | b3 | H | H | a5 |
| 2-1-039 | N—Ph | N—Ph | H | H | H | a6 |
| 2-1-040 | N—Ph | N—Ph | t-bu | H | H | a6 |
| 2-1-041 | N—Ph | N—Ph | methyl | H | H | a6 |
| 2-1-042 | N—Ph | N—Ph | b1 | H | H | a6 |
| 2-1-043 | N—Ph | N—Ph | b2 | H | H | a6 |
| 2-1-044 | N—Ph | N—Ph | b3 | H | H | a6 |
| 2-1-045 | N—Ph | N—Ph | H | H | a1 | H |
| 2-1-046 | N—Ph | N—Ph | t-bu | H | a1 | H |
| 2-1-047 | N—Ph | N—Ph | methyl | H | a1 | H |
| 2-1-048 | N—Ph | N—Ph | b1 | H | a1 | H |
| 2-1-049 | N—Ph | N—Ph | b2 | H | a1 | H |
| 2-1-050 | N—Ph | N—Ph | b3 | H | a1 | H |
| 2-1-051 | N—Ph | N—Ph | H | H | a2 | H |
| 2-1-052 | N—Ph | N—Ph | t-bu | H | a2 | H |
| 2-1-053 | N—Ph | N—Ph | methyl | H | a2 | H |
| 2-1-054 | N—Ph | N—Ph | b1 | H | a2 | H |
| 2-1-055 | N—Ph | N—Ph | b2 | H | a2 | H |
| 2-1-056 | N—Ph | N—Ph | b3 | H | a2 | H |
| 2-1-057 | N—Ph | N—Ph | H | H | a3 | H |
| 2-1-058 | N—Ph | N—Ph | t-bu | H | a3 | H |
| 2-1-059 | N—Ph | N—Ph | methyl | H | a3 | H |
| 2-1-060 | N—Ph | N—Ph | b1 | H | a3 | H |
| 2-1-061 | N—Ph | N—Ph | b2 | H | a3 | H |
| 2-1-062 | N—Ph | N—Ph | b3 | H | a3 | H |
| 2-1-063 | N—Ph | N—Ph | H | H | a4 | H |
| 2-1-064 | N—Ph | N—Ph | t-bu | H | a4 | H |
| 2-1-065 | N—Ph | N—Ph | methyl | H | a4 | H |
| 2-1-066 | N—Ph | N—Ph | b1 | H | a4 | H |
| 2-1-067 | N—Ph | N—Ph | b2 | H | a4 | H |
| 2-1-068 | N—Ph | N—Ph | b3 | H | a4 | H |
| 2-1-069 | N—Ph | N—Ph | H | H | a5 | H |
| 2-1-070 | N—Ph | N—Ph | t-bu | H | a5 | H |
| 2-1-071 | N—Ph | N—Ph | methyl | H | a5 | H |
| 2-1-072 | N—Ph | N—Ph | b1 | H | a5 | H |
| 2-1-073 | N—Ph | N—Ph | b2 | H | a5 | H |
| 2-1-074 | N—Ph | N—Ph | b3 | H | a5 | H |
| 2-1-075 | N—Ph | N—Ph | H | H | a6 | H |
| 2-1-076 | N—Ph | N—Ph | t-bu | H | a6 | H |
| 2-1-077 | N—Ph | N—Ph | methyl | H | a6 | H |
| 2-1-078 | N—Ph | N—Ph | b1 | H | a6 | H |
| 2-1-079 | N—Ph | N—Ph | b2 | H | a6 | H |
| 2-1-080 | N—Ph | N—Ph | b3 | H | a6 | H |
| 2-1-081 | N—Ph | N—Ph | H | H | b1 | H |
| 2-1-082 | N—Ph | N—Ph | t-bu | H | b1 | H |
| 2-1-083 | N—Ph | N—Ph | methyl | H | b1 | H |
| 2-1-084 | N—Ph | N—Ph | b1 | H | b1 | H |
| 2-1-085 | N—Ph | N—Ph | b2 | H | b1 | H |
| 2-1-086 | N—Ph | N—Ph | b3 | H | b1 | H |
| 2-1-087 | N—Ph | N—Ph | H | H | b2 | H |
| 2-1-088 | N—Ph | N—Ph | t-bu | H | b2 | H |
| 2-1-089 | N—Ph | N—Ph | methyl | H | b2 | H |
| 2-1-090 | N—Ph | N—Ph | b1 | H | b2 | H |
| 2-1-091 | N—Ph | N—Ph | b2 | H | b2 | H |
| 2-1-092 | N—Ph | N—Ph | b3 | H | b2 | H |
| 2-1-093 | N—Ph | N—Ph | H | H | b3 | H |
| 2-1-094 | N—Ph | N—Ph | t-bu | H | b3 | H |
| 2-1-095 | N—Ph | N—Ph | methyl | H | b3 | H |
| 2-1-096 | N—Ph | N—Ph | b1 | H | b3 | H |
| 2-1-097 | N—Ph | N—Ph | b2 | H | b3 | H |
| 2-1-098 | N—Ph | N—Ph | b3 | H | b3 | H |
| 2-1-099 | N—Ph | N—Ph | H | H | b4 | H |
| 2-1-100 | N—Ph | N—Ph | t-bu | H | b4 | H |
| 2-1-101 | N—Ph | N—Ph | methyl | H | b4 | H |
| 2-1-102 | N—Ph | N—Ph | b1 | H | b4 | H |
| 2-1-103 | N—Ph | N—Ph | b2 | H | b4 | H |
| 2-1-104 | N—Ph | N—Ph | b3 | H | b4 | H |
| 2-1-105 | N—Ph | N—Ph | H | H | b5 | H |
| 2-1-106 | N—Ph | N—Ph | t-bu | H | b5 | H |
| 2-1-107 | N—Ph | N—Ph | methyl | H | b5 | H |
| 2-1-108 | N—Ph | N—Ph | b1 | H | b5 | H |
| 2-1-109 | N—Ph | N—Ph | b2 | H | b5 | H |
| 2-1-110 | N—Ph | N—Ph | b3 | H | b5 | H |
| 2-1-111 | N—Ph | N—Ph | H | H | b6 | H |
| 2-1-112 | N—Ph | N—Ph | t-bu | H | b6 | H |
| 2-1-113 | N—Ph | N—Ph | methyl | H | b6 | H |
| 2-1-114 | N—Ph | N—Ph | b1 | H | b6 | H |
| 2-1-115 | N—Ph | N—Ph | b2 | H | b6 | H |
| 2-1-116 | N—Ph | N—Ph | b3 | H | b6 | H |
| 2-1-117 | N—Ph | N—Ph | H | H | b7 | H |
| 2-1-118 | N—Ph | N—Ph | t-bu | H | b7 | H |
| 2-1-119 | N—Ph | N—Ph | methyl | H | b7 | H |
| 2-1-120 | N—Ph | N—Ph | b1 | H | b7 | H |
| 2-1-121 | N—Ph | N—Ph | b2 | H | b7 | H |
| 2-1-122 | N—Ph | N—Ph | b3 | H | b7 | H |
| 2-1-123 | N—Ph | N—Ph | H | H | b8 | H |
| 2-1-124 | N—Ph | N—Ph | t-bu | H | b8 | H |
| 2-1-125 | N—Ph | N—Ph | methyl | H | b8 | H |
| 2-1-126 | N—Ph | N—Ph | b1 | H | b8 | H |
| 2-1-127 | N—Ph | N—Ph | b2 | H | b8 | H |
| 2-1-128 | N—Ph | N—Ph | b3 | H | b8 | H |
| 2-1-129 | N—Ph | N—Ph | H | H | b9 | H |
| 2-1-130 | N—Ph | N—Ph | t-bu | H | b9 | H |
| 2-1-131 | N—Ph | N—Ph | methyl | H | b9 | H |
| 2-1-132 | N—Ph | N—Ph | b1 | H | b9 | H |
| 2-1-133 | N—Ph | N—Ph | b2 | H | b9 | H |
| 2-1-134 | N—Ph | N—Ph | b3 | H | b9 | H |
| 2-1-135 | N—Ph | N—Ph | H | H | b10 | H |
| 2-1-136 | N—Ph | N—Ph | t-bu | H | b10 | H |
| 2-1-137 | N—Ph | N—Ph | methyl | H | b10 | H |
| 2-1-138 | N—Ph | N—Ph | b1 | H | b10 | H |
| 2-1-139 | N—Ph | N—Ph | b2 | H | b10 | H |
| 2-1-140 | N—Ph | N—Ph | b3 | H | b10 | H |
| 2-1-141 | N—Ph | N—Ph | H | H | a1 | a1 |
| 2-1-142 | N—Ph | N—Ph | t-bu | H | a1 | a1 |
| 2-1-143 | N—Ph | N—Ph | methyl | H | a1 | a1 |
| 2-1-144 | N—Ph | N—Ph | b1 | H | a1 | a1 |
| 2-1-145 | N—Ph | N—Ph | b2 | H | a1 | a1 |
| 2-1-146 | N—Ph | N—Ph | b3 | H | a1 | a1 |
| 2-1-147 | N—Ph | N—Ph | H | H | a2 | a2 |
| 2-1-148 | N—Ph | N—Ph | t-bu | H | a2 | a2 |
| 2-1-149 | N—Ph | N—Ph | methyl | H | a2 | a2 |
| 2-1-150 | N—Ph | N—Ph | b1 | H | a2 | a2 |
| 2-1-151 | N—Ph | N—Ph | b2 | H | a2 | a2 |
| 2-1-152 | N—Ph | N—Ph | b3 | H | a2 | a2 |
| 2-1-153 | N—Ph | N—Ph | H | H | a3 | a3 |
| 2-1-154 | N—Ph | N—Ph | t-bu | H | a3 | a3 |
| 2-1-155 | N—Ph | N—Ph | methyl | H | a3 | a3 |
| 2-1-156 | N—Ph | N—Ph | b1 | H | a3 | a3 |
| 2-1-157 | N—Ph | N—Ph | b2 | H | a3 | a3 |
| 2-1-158 | N—Ph | N—Ph | b3 | H | a3 | a3 |
| 2-1-159 | N—Ph | N—Ph | H | H | b1 | b1 |
| 2-1-160 | N—Ph | N—Ph | t-bu | H | b1 | b1 |
| 2-1-161 | N—Ph | N—Ph | methyl | H | b1 | b1 |
| 2-1-162 | N—Ph | N—Ph | b1 | H | b1 | b1 |
| 2-1-163 | N—Ph | N—Ph | b2 | H | b1 | b1 |
| 2-1-164 | N—Ph | N—Ph | b3 | H | b1 | b1 |
| 2-1-165 | N—Ph | N—Ph | H | H | b2 | b2 |
| 2-1-166 | N—Ph | N—Ph | t-bu | H | b2 | b2 |
| 2-1-167 | N—Ph | N—Ph | methyl | H | b2 | b2 |
| 2-1-168 | N—Ph | N—Ph | b1 | H | b2 | b2 |
| 2-1-169 | N—Ph | N—Ph | b2 | H | b2 | b2 |
| 2-1-170 | N—Ph | N—Ph | b3 | H | b2 | b2 |
| 2-1-171 | N—Ph | N—Ph | H | H | b3 | b3 |
| 2-1-172 | N—Ph | N—Ph | t-bu | H | b3 | b3 |
| 2-1-173 | N—Ph | N—Ph | methyl | H | b3 | b3 |
| 2-1-174 | N—Ph | N—Ph | b1 | H | b3 | b3 |
| 2-1-175 | N—Ph | N—Ph | b2 | H | b3 | b3 |
| 2-1-176 | N—Ph | N—Ph | b3 | H | b3 | b3 |
| 2-1-177 | N—Ph | N—Ph | H | H | b5 | b5 |
| 2-1-178 | N—Ph | N—Ph | t-bu | H | b5 | b5 |
| 2-1-179 | N—Ph | N—Ph | methyl | H | b5 | b5 |
| 2-1-180 | N—Ph | N—Ph | b1 | H | b5 | b5 |
| 2-1-181 | N—Ph | N—Ph | b2 | H | b5 | b5 |
| 2-1-182 | N—Ph | N—Ph | b3 | H | b5 | b5 |
| 2-1-183 | N—Ph | N—Ph | H | H | b6 | b6 |
| 2-1-184 | N—Ph | N—Ph | t-bu | H | b6 | b6 |
| 2-1-185 | N—Ph | N—Ph | methyl | H | b6 | b6 |
| 2-1-186 | N—Ph | N—Ph | b1 | H | b6 | b6 |
| 2-1-187 | N—Ph | N—Ph | b2 | H | b6 | b6 |
| 2-1-188 | N—Ph | N—Ph | b3 | H | b6 | b6 |
| 2-1-189 | N—Ph | N—Ph | H | H | b7 | b7 |
| 2-1-190 | N—Ph | N—Ph | t-bu | H | b7 | b7 |
| 2-1-191 | N—Ph | N—Ph | methyl | H | b7 | b7 |
| 2-1-192 | N—Ph | N—Ph | b1 | H | b7 | b7 |
| 2-1-193 | N—Ph | N—Ph | b2 | H | b7 | b7 |
| 2-1-194 | N—Ph | N—Ph | b3 | H | b7 | b7 |
| 2-1-195 | N-tolyl | N-tolyl | H | H | H | H |
| 2-1-196 | N-tolyl | N-tolyl | t-bu | H | H | H |
| 2-1-197 | N-tolyl | N-tolyl | methyl | H | H | H |
| 2-1-198 | N-tolyl | N-tolyl | t-bu | t-bu | t-bu | t-bu |
| 2-1-199 | N-tolyl | N-tolyl | methyl | methyl | methyl | methyl |
| 2-1-200 | N-tolyl | N-tolyl | b1 | H | H | H |
| 2-1-201 | N-tolyl | N-tolyl | b2 | H | H | H |
| 2-1-202 | N-tolyl | N-tolyl | b3 | H | H | H |
| 2-1-203 | N-tolyl | N-tolyl | H | H | H | a1 |
| 2-1-204 | N-tolyl | N-tolyl | t-bu | H | H | a1 |
| 2-1-205 | N-tolyl | N-tolyl | methyl | H | H | a1 |
| 2-1-206 | N-tolyl | N-tolyl | b1 | H | H | a1 |
| 2-1-207 | N-tolyl | N-tolyl | b2 | H | H | a1 |
| 2-1-208 | N-tolyl | N-tolyl | b3 | H | H | a1 |
| 2-1-209 | N-tolyl | N-tolyl | H | H | H | a2 |
| 2-1-210 | N-tolyl | N-tolyl | t-bu | H | H | a2 |
| 2-1-211 | N-tolyl | N-tolyl | methyl | H | H | a2 |
| 2-1-212 | N-tolyl | N-tolyl | b1 | H | H | a2 |
| 2-1-213 | N-tolyl | N-tolyl | b2 | H | H | a2 |
| 2-1-214 | N-tolyl | N-tolyl | b3 | H | H | a2 |
| 2-1-215 | N-tolyl | N-tolyl | H | H | H | a3 |
| 2-1-216 | N-tolyl | N-tolyl | t-bu | H | H | a3 |
| 2-1-217 | N-tolyl | N-tolyl | methyl | H | H | a3 |
| 2-1-218 | N-tolyl | N-tolyl | b1 | H | H | a3 |
| 2-1-219 | N-tolyl | N-tolyl | b2 | H | H | a3 |
| 2-1-220 | N-tolyl | N-tolyl | b3 | H | H | a3 |
| 2-1-221 | N-tolyl | N-tolyl | H | H | H | a4 |
| 2-1-222 | N-tolyl | N-tolyl | t-bu | H | H | a4 |
| 2-1-223 | N-tolyl | N-tolyl | methyl | H | H | a4 |
| 2-1-224 | N-tolyl | N-tolyl | b1 | H | H | a4 |
| 2-1-225 | N-tolyl | N-tolyl | b2 | H | H | a4 |
| 2-1-226 | N-tolyl | N-tolyl | b3 | H | H | a4 |
| 2-1-227 | N-tolyl | N-tolyl | H | H | H | a5 |
| 2-1-228 | N-tolyl | N-tolyl | t-bu | H | H | a5 |
| 2-1-229 | N-tolyl | N-tolyl | methyl | H | H | a5 |
| 2-1-230 | N-tolyl | N-tolyl | b1 | H | H | a5 |
| 2-1-231 | N-tolyl | N-tolyl | b2 | H | H | a5 |
| 2-1-232 | N-tolyl | N-tolyl | b3 | H | H | a5 |
| 2-1-233 | N-tolyl | N-tolyl | H | H | H | a6 |
| 2-1-234 | N-tolyl | N-tolyl | t-bu | H | H | a6 |
| 2-1-235 | N-tolyl | N-tolyl | methyl | H | H | a6 |
| 2-1-236 | N-tolyl | N-tolyl | b1 | H | H | a6 |
| 2-1-237 | N-tolyl | N-tolyl | b2 | H | H | a6 |
| 2-1-238 | N-tolyl | N-tolyl | b3 | H | H | a6 |
| 2-1-239 | N-tolyl | N-tolyl | H | H | a1 | H |
| 2-1-240 | N-tolyl | N-tolyl | t-bu | H | a1 | H |
| 2-1-241 | N-tolyl | N-tolyl | methyl | H | a1 | H |
| 2-1-242 | N-tolyl | N-tolyl | b1 | H | a1 | H |
| 2-1-243 | N-tolyl | N-tolyl | b2 | H | a1 | H |
| 2-1-244 | N-tolyl | N-tolyl | b3 | H | a1 | H |
| 2-1-245 | N-tolyl | N-tolyl | H | H | a2 | H |
| 2-1-246 | N-tolyl | N-tolyl | t-bu | H | a2 | H |
| 2-1-247 | N-tolyl | N-tolyl | methyl | H | a2 | H |
| 2-1-248 | N-tolyl | N-tolyl | b1 | H | a2 | H |
| 2-1-249 | N-tolyl | N-tolyl | b2 | H | a2 | H |
| 2-1-250 | N-tolyl | N-tolyl | b3 | H | a2 | H |
| 2-1-251 | N-tolyl | N-tolyl | H | H | a3 | H |
| 2-1-252 | N-tolyl | N-tolyl | t-bu | H | a3 | H |
| 2-1-253 | N-tolyl | N-tolyl | methyl | H | a3 | H |
| 2-1-254 | N-tolyl | N-tolyl | b1 | H | a3 | H |
| 2-1-255 | N-tolyl | N-tolyl | b2 | H | a3 | H |
| 2-1-256 | N-tolyl | N-tolyl | b3 | H | a3 | H |
| 2-1-257 | N-tolyl | N-tolyl | H | H | a4 | H |
| 2-1-258 | N-tolyl | N-tolyl | t-bu | H | a4 | H |
| 2-1-259 | N-tolyl | N-tolyl | methyl | H | a4 | H |
| 2-1-260 | N-tolyl | N-tolyl | b1 | H | a4 | H |
| 2-1-261 | N-tolyl | N-tolyl | b2 | H | a4 | H |
| 2-1-262 | N-tolyl | N-tolyl | b3 | H | a4 | H |
| 2-1-263 | N-tolyl | N-tolyl | H | H | a5 | H |
| 2-1-264 | N-tolyl | N-tolyl | t-bu | H | a5 | H |
| 2-1-265 | N-tolyl | N-tolyl | methyl | H | a5 | H |
| 2-1-266 | N-tolyl | N-tolyl | b1 | H | a5 | H |
| 2-1-267 | N-tolyl | N-tolyl | b2 | H | a5 | H |
| 2-1-268 | N-tolyl | N-tolyl | b3 | H | a5 | H |
| 2-1-269 | N-tolyl | N-tolyl | H | H | a6 | H |
| 2-1-270 | N-tolyl | N-tolyl | t-bu | H | a6 | H |
| 2-1-271 | N-tolyl | N-tolyl | methyl | H | a6 | H |
| 2-1-272 | N-tolyl | N-tolyl | b1 | H | a6 | H |
| 2-1-273 | N-tolyl | N-tolyl | b2 | H | a6 | H |
| 2-1-274 | N-tolyl | N-tolyl | b3 | H | a6 | H |
| 2-1-275 | N-tolyl | N-tolyl | H | H | b1 | H |
| 2-1-276 | N-tolyl | N-tolyl | t-bu | H | b1 | H |
| 2-1-277 | N-tolyl | N-tolyl | methyl | H | b1 | H |
| 2-1-278 | N-tolyl | N-tolyl | b1 | H | b1 | H |
| 2-1-279 | N-tolyl | N-tolyl | b2 | H | b1 | H |
| 2-1-280 | N-tolyl | N-tolyl | b3 | H | b1 | H |
| 2-1-281 | N-tolyl | N-tolyl | H | H | b2 | H |
| 2-1-282 | N-tolyl | N-tolyl | t-bu | H | b2 | H |
| 2-1-283 | N-tolyl | N-tolyl | methyl | H | b2 | H |
| 2-1-284 | N-tolyl | N-tolyl | b1 | H | b2 | H |
| 2-1-285 | N-tolyl | N-tolyl | b2 | H | b2 | H |
| 2-1-286 | N-tolyl | N-tolyl | b3 | H | b2 | H |
| 2-1-287 | N-tolyl | N-tolyl | H | H | b3 | H |
| 2-1-288 | N-tolyl | N-tolyl | t-bu | H | b3 | H |
| 2-1-289 | N-tolyl | N-tolyl | methyl | H | b3 | H |
| 2-1-290 | N-tolyl | N-tolyl | b1 | H | b3 | H |
| 2-1-291 | N-tolyl | N-tolyl | b2 | H | b3 | H |
| 2-1-292 | N-tolyl | N-tolyl | b3 | H | b3 | H |
| 2-1-293 | N-tolyl | N-tolyl | H | H | b4 | H |
| 2-1-294 | N-tolyl | N-tolyl | t-bu | H | b4 | H |
| 2-1-295 | N-tolyl | N-tolyl | methyl | H | b4 | H |
| 2-1-296 | N-tolyl | N-tolyl | b1 | H | b4 | H |
| 2-1-297 | N-tolyl | N-tolyl | b2 | H | b4 | H |
| 2-1-298 | N-tolyl | N-tolyl | b3 | H | b4 | H |
| 2-1-299 | N-tolyl | N-tolyl | H | H | b5 | H |
| 2-1-300 | N-tolyl | N-tolyl | t-bu | H | b5 | H |
| 2-1-301 | N-tolyl | N-tolyl | methyl | H | b5 | H |
| 2-1-302 | N-tolyl | N-tolyl | b1 | H | b5 | H |
| 2-1-303 | N-tolyl | N-tolyl | b2 | H | b5 | H |
| 2-1-304 | N-tolyl | N-tolyl | b3 | H | b5 | H |
| 2-1-305 | N-tolyl | N-tolyl | H | H | b6 | H |
| 2-1-306 | N-tolyl | N-tolyl | t-bu | H | b6 | H |
| 2-1-307 | N-tolyl | N-tolyl | methyl | H | b6 | H |
| 2-1-308 | N-tolyl | N-tolyl | b1 | H | b6 | H |
| 2-1-309 | N-tolyl | N-tolyl | b2 | H | b6 | H |
| 2-1-310 | N-tolyl | N-tolyl | b3 | H | b6 | H |
| 2-1-311 | N-tolyl | N-tolyl | H | H | b7 | H |
| 2-1-312 | N-tolyl | N-tolyl | t-bu | H | b7 | H |
| 2-1-313 | N-tolyl | N-tolyl | methyl | H | b7 | H |
| 2-1-314 | N-tolyl | N-tolyl | b1 | H | b7 | H |
| 2-1-315 | N-tolyl | N-tolyl | b2 | H | b7 | H |
| 2-1-316 | N-tolyl | N-tolyl | b3 | H | b7 | H |
| 2-1-317 | N-tolyl | N-tolyl | H | H | b8 | H |
| 2-1-318 | N-tolyl | N-tolyl | t-bu | H | b8 | H |
| 2-1-319 | N-tolyl | N-tolyl | methyl | H | b8 | H |
| 2-1-320 | N-tolyl | N-tolyl | b1 | H | b8 | H |
| 2-1-321 | N-tolyl | N-tolyl | b2 | H | b8 | H |
| 2-1-322 | N-tolyl | N-tolyl | b3 | H | b8 | H |
| 2-1-323 | N-tolyl | N-tolyl | H | H | b9 | H |
| 2-1-324 | N-tolyl | N-tolyl | t-bu | H | b9 | H |
| 2-1-325 | N-tolyl | N-tolyl | methyl | H | b9 | H |
| 2-1-326 | N-tolyl | N-tolyl | b1 | H | b9 | H |
| 2-1-327 | N-tolyl | N-tolyl | b2 | H | b9 | H |
| 2-1-328 | N-tolyl | N-tolyl | b3 | H | b9 | H |
| 2-1-329 | N-tolyl | N-tolyl | H | H | b10 | H |
| 2-1-330 | N-tolyl | N-tolyl | t-bu | H | b10 | H |
| 2-1-331 | N-tolyl | N-tolyl | methyl | H | b10 | H |
| 2-1-332 | N-tolyl | N-tolyl | b1 | H | b10 | H |
| 2-1-333 | N-tolyl | N-tolyl | b2 | H | b10 | H |
| 2-1-334 | N-tolyl | N-tolyl | b3 | H | b10 | H |
| 2-1-335 | N-tolyl | N-tolyl | H | H | a1 | a1 |
| 2-1-336 | N-tolyl | N-tolyl | t-bu | H | a1 | a1 |
| 2-1-337 | N-tolyl | N-tolyl | methyl | H | a1 | a1 |
| 2-1-338 | N-tolyl | N-tolyl | b1 | H | a1 | a1 |
| 2-1-339 | N-tolyl | N-tolyl | b2 | H | a1 | a1 |
| 2-1-340 | N-tolyl | N-tolyl | b3 | H | a1 | a1 |
| 2-1-341 | N-tolyl | N-tolyl | H | H | a2 | a2 |
| 2-1-342 | N-tolyl | N-tolyl | t-bu | H | a2 | a2 |
| 2-1-343 | N-tolyl | N-tolyl | methyl | H | a2 | a2 |
| 2-1-344 | N-tolyl | N-tolyl | b1 | H | a2 | a2 |
| 2-1-345 | N-tolyl | N-tolyl | b2 | H | a2 | a2 |
| 2-1-346 | N-tolyl | N-tolyl | b3 | H | a2 | a2 |
| 2-1-347 | N-tolyl | N-tolyl | H | H | a3 | a3 |
| 2-1-348 | N-tolyl | N-tolyl | t-bu | H | a3 | a3 |
| 2-1-349 | N-tolyl | N-tolyl | methyl | H | a3 | a3 |
| 2-1-350 | N-tolyl | N-tolyl | b1 | H | a3 | a3 |
| 2-1-351 | N-tolyl | N-tolyl | b2 | H | a3 | a3 |
| 2-1-352 | N-tolyl | N-tolyl | b3 | H | a3 | a3 |
| 2-1-353 | N-tolyl | N-tolyl | H | H | b1 | b1 |
| 2-1-354 | N-tolyl | N-tolyl | t-bu | H | b1 | b1 |
| 2-1-355 | N-tolyl | N-tolyl | methyl | H | b1 | b1 |
| 2-1-356 | N-tolyl | N-tolyl | b1 | H | b1 | b1 |
| 2-1-357 | N-tolyl | N-tolyl | b2 | H | b1 | b1 |
| 2-1-358 | N-tolyl | N-tolyl | b3 | H | b1 | b1 |
| 2-1-359 | N-tolyl | N-tolyl | H | H | b2 | b2 |
| 2-1-360 | N-tolyl | N-tolyl | t-bu | H | b2 | b2 |
| 2-1-361 | N-tolyl | N-tolyl | methyl | H | b2 | b2 |
| 2-1-362 | N-tolyl | N-tolyl | b1 | H | b2 | b2 |
| 2-1-363 | N-tolyl | N-tolyl | b2 | H | b2 | b2 |
| 2-1-364 | N-tolyl | N-tolyl | b3 | H | b2 | b2 |
| 2-1-365 | N-tolyl | N-tolyl | H | H | b3 | b3 |
| 2-1-366 | N-tolyl | N-tolyl | t-bu | H | b3 | b3 |
| 2-1-367 | N-tolyl | N-tolyl | methyl | H | b3 | b3 |
| 2-1-368 | N-tolyl | N-tolyl | b1 | H | b3 | b3 |
| 2-1-369 | N-tolyl | N-tolyl | b2 | H | b3 | b3 |
| 2-1-370 | N-tolyl | N-tolyl | b3 | H | b3 | b3 |
| 2-1-371 | N-tolyl | N-tolyl | H | H | b5 | b5 |
| 2-1-372 | N-tolyl | N-tolyl | t-bu | H | b5 | b5 |
| 2-1-373 | N-tolyl | N-tolyl | methyl | H | b5 | b5 |
| 2-1-374 | N-tolyl | N-tolyl | b1 | H | b5 | b5 |
| 2-1-375 | N-tolyl | N-tolyl | b2 | H | b5 | b5 |
| 2-1-376 | N-tolyl | N-tolyl | b3 | H | b5 | b5 |
| 2-1-377 | N-tolyl | N-tolyl | H | H | b6 | b6 |
| 2-1-378 | N-tolyl | N-tolyl | t-bu | H | b6 | b6 |
| 2-1-379 | N-tolyl | N-tolyl | methyl | H | b6 | b6 |
| 2-1-380 | N-tolyl | N-tolyl | b1 | H | b6 | b6 |
| 2-1-381 | N-tolyl | N-tolyl | b2 | H | b6 | b6 |
| 2-1-382 | N-tolyl | N-tolyl | b3 | H | b6 | b6 |
| 2-1-383 | N-tolyl | N-tolyl | H | H | b7 | b7 |
| 2-1-384 | N-tolyl | N-tolyl | t-bu | H | b7 | b7 |
| 2-1-385 | N-tolyl | N-tolyl | methyl | H | b7 | b7 |
| 2-1-386 | N-tolyl | N-tolyl | b1 | H | b7 | b7 |
| 2-1-387 | N-tolyl | N-tolyl | b2 | H | b7 | b7 |
| 2-1-388 | N-tolyl | N-tolyl | b3 | H | b7 | b7 |
| 2-1-389 | N-Biph | N-Biph | H | H | H | H |
| 2-1-390 | N-Biph | N-Biph | t-bu | H | H | H |
| 2-1-391 | N-Biph | N-Biph | methyl | H | H | H |
| 2-1-392 | N-Biph | N-Biph | t-bu | t-bu | t-bu | t-bu |
| 2-1-393 | N-Biph | N-Biph | methyl | methyl | methyl | methyl |
| 2-1-394 | N-Biph | N-Biph | b1 | H | H | H |
| 2-1-395 | N-Biph | N-Biph | b2 | H | H | H |
| 2-1-396 | N-Biph | N-Biph | b3 | H | H | H |
| 2-1-397 | N-Biph | N-Biph | H | H | H | a1 |
| 2-1-398 | N-Biph | N-Biph | t-bu | H | H | a1 |
| 2-1-399 | N-Biph | N-Biph | methyl | H | H | a1 |
| 2-1-400 | N-Biph | N-Biph | b1 | H | H | a1 |
| 2-1-401 | N-Biph | N-Biph | b2 | H | H | a1 |
| 2-1-402 | N-Biph | N-Biph | b3 | H | H | a1 |
| 2-1-403 | N-Biph | N-Biph | H | H | H | a2 |
| 2-1-404 | N-Biph | N-Biph | t-bu | H | H | a2 |
| 2-1-405 | N-Biph | N-Biph | methyl | H | H | a2 |
| 2-1-406 | N-Biph | N-Biph | b1 | H | H | a2 |
| 2-1-407 | N-Biph | N-Biph | b2 | H | H | a2 |
| 2-1-408 | N-Biph | N-Biph | b3 | H | H | a2 |
| 2-1-409 | N-Biph | N-Biph | H | H | H | a3 |
| 2-1-410 | N-Biph | N-Biph | t-bu | H | H | a3 |
| 2-1-411 | N-Biph | N-Biph | methyl | H | H | a3 |
| 2-1-412 | N-Biph | N-Biph | b1 | H | H | a3 |
| 2-1-413 | N-Biph | N-Biph | b2 | H | H | a3 |
| 2-1-414 | N-Biph | N-Biph | b3 | H | H | a3 |
| 2-1-415 | N-Biph | N-Biph | H | H | H | a4 |
| 2-1-416 | N-Biph | N-Biph | t-bu | H | H | a4 |
| 2-1-417 | N-Biph | N-Biph | methyl | H | H | a4 |
| 2-1-418 | N-Biph | N-Biph | b1 | H | H | a4 |
| 2-1-419 | N-Biph | N-Biph | b2 | H | H | a4 |
| 2-1-420 | N-Biph | N-Biph | b3 | H | H | a4 |
| 2-1-421 | N-Biph | N-Biph | H | H | H | a5 |
| 2-1-422 | N-Biph | N-Biph | t-bu | H | H | a5 |
| 2-1-423 | N-Biph | N-Biph | methyl | H | H | a5 |
| 2-1-424 | N-Biph | N-Biph | b1 | H | H | a5 |
| 2-1-425 | N-Biph | N-Biph | b2 | H | H | a5 |
| 2-1-426 | N-Biph | N-Biph | b3 | H | H | a5 |
| 2-1-427 | N-Biph | N-Biph | H | H | H | a6 |
| 2-1-428 | N-Biph | N-Biph | t-bu | H | H | a6 |
| 2-1-429 | N-Biph | N-Biph | methyl | H | H | a6 |
| 2-1-430 | N-Biph | N-Biph | b1 | H | H | a6 |
| 2-1-431 | N-Biph | N-Biph | b2 | H | H | a6 |
| 2-1-432 | N-Biph | N-Biph | b3 | H | H | a6 |
| 2-1-433 | N-Biph | N-Biph | H | H | a1 | H |
| 2-1-434 | N-Biph | N-Biph | t-bu | H | a1 | H |
| 2-1-435 | N-Biph | N-Biph | methyl | H | a1 | H |
| 2-1-436 | N-Biph | N-Biph | b1 | H | a1 | H |
| 2-1-437 | N-Biph | N-Biph | b2 | H | a1 | H |
| 2-1-438 | N-Biph | N-Biph | b3 | H | a1 | H |
| 2-1-439 | N-Biph | N-Biph | H | H | a2 | H |
| 2-1-440 | N-Biph | N-Biph | t-bu | H | a2 | H |
| 2-1-441 | N-Biph | N-Biph | methyl | H | a2 | H |
| 2-1-442 | N-Biph | N-Biph | b1 | H | a2 | H |
| 2-1-443 | N-Biph | N-Biph | b2 | H | a2 | H |
| 2-1-444 | N-Biph | N-Biph | b3 | H | a2 | H |
| 2-1-445 | N-Biph | N-Biph | H | H | a3 | H |
| 2-1-446 | N-Biph | N-Biph | t-bu | H | a3 | H |
| 2-1-447 | N-Biph | N-Biph | methyl | H | a3 | H |
| 2-1-448 | N-Biph | N-Biph | b1 | H | a3 | H |
| 2-1-449 | N-Biph | N-Biph | b2 | H | a3 | H |
| 2-1-450 | N-Biph | N-Biph | b3 | H | a3 | H |
| 2-1-451 | N-Biph | N-Biph | H | H | a4 | H |
| 2-1-452 | N-Biph | N-Biph | t-bu | H | a4 | H |
| 2-1-453 | N-Biph | N-Biph | methyl | H | a4 | H |
| 2-1-454 | N-Biph | N-Biph | b1 | H | a4 | H |
| 2-1-455 | N-Biph | N-Biph | b2 | H | a4 | H |
| 2-1-456 | N-Biph | N-Biph | b3 | H | a4 | H |
| 2-1-457 | N-Biph | N-Biph | H | H | a5 | H |
| 2-1-458 | N-Biph | N-Biph | t-bu | H | a5 | H |
| 2-1-459 | N-Biph | N-Biph | methyl | H | a5 | H |
| 2-1-460 | N-Biph | N-Biph | b1 | H | a5 | H |
| 2-1-461 | N-Biph | N-Biph | b2 | H | a5 | H |
| 2-1-462 | N-Biph | N-Biph | b3 | H | a5 | H |
| 2-1-463 | N-Biph | N-Biph | H | H | a6 | H |
| 2-1-464 | N-Biph | N-Biph | t-bu | H | a6 | H |
| 2-1-465 | N-Biph | N-Biph | methyl | H | a6 | H |
| 2-1-466 | N-Biph | N-Biph | b1 | H | a6 | H |
| 2-1-467 | N-Biph | N-Biph | b2 | H | a6 | H |
| 2-1-468 | N-Biph | N-Biph | b3 | H | a6 | H |
| 2-1-469 | N-Biph | N-Biph | H | H | b1 | H |
| 2-1-470 | N-Biph | N-Biph | t-bu | H | b1 | H |
| 2-1-471 | N-Biph | N-Biph | methyl | H | b1 | H |
| 2-1-472 | N-Biph | N-Biph | b1 | H | b1 | H |
| 2-1-473 | N-Biph | N-Biph | b2 | H | b1 | H |
| 2-1-474 | N-Biph | N-Biph | b3 | H | b1 | H |
| 2-1-475 | N-Biph | N-Biph | H | H | b2 | H |
| 2-1-476 | N-Biph | N-Biph | t-bu | H | b2 | H |
| 2-1-477 | N-Biph | N-Biph | methyl | H | b2 | H |
| 2-1-478 | N-Biph | N-Biph | b1 | H | b2 | H |
| 2-1-479 | N-Biph | N-Biph | b2 | H | b2 | H |
| 2-1-480 | N-Biph | N-Biph | b3 | H | b2 | H |
| 2-1-481 | N-Biph | N-Biph | H | H | b3 | H |
| 2-1-482 | N-Biph | N-Biph | t-bu | H | b3 | H |
| 2-1-483 | N-Biph | N-Biph | methyl | H | b3 | H |
| 2-1-484 | N-Biph | N-Biph | b1 | H | b3 | H |
| 2-1-485 | N-Biph | N-Biph | b2 | H | b3 | H |
| 2-1-486 | N-Biph | N-Biph | b3 | H | b3 | H |
| 2-1-487 | N-Biph | N-Biph | H | H | b4 | H |
| 2-1-488 | N-Biph | N-Biph | t-bu | H | b4 | H |
| 2-1-489 | N-Biph | N-Biph | methyl | H | b4 | H |
| 2-1-490 | N-Biph | N-Biph | b1 | H | b4 | H |
| 2-1-491 | N-Biph | N-Biph | b2 | H | b4 | H |
| 2-1-492 | N-Biph | N-Biph | b3 | H | b4 | H |
| 2-1-493 | N-Biph | N-Biph | H | H | b5 | H |
| 2-1-494 | N-Biph | N-Biph | t-bu | H | b5 | H |
| 2-1-495 | N-Biph | N-Biph | methyl | H | b5 | H |
| 2-1-496 | N-Biph | N-Biph | b1 | H | b5 | H |
| 2-1-497 | N-Biph | N-Biph | b2 | H | b5 | H |
| 2-1-498 | N-Biph | N-Biph | b3 | H | b5 | H |
| 2-1-499 | N-Biph | N-Biph | H | H | b6 | H |
| 2-1-500 | N-Biph | N-Biph | t-bu | H | b6 | H |
| 2-1-501 | N-Biph | N-Biph | methyl | H | b6 | H |
| 2-1-502 | N-Biph | N-Biph | b1 | H | b6 | H |
| 2-1-503 | N-Biph | N-Biph | b2 | H | b6 | H |
| 2-1-504 | N-Biph | N-Biph | b3 | H | b6 | H |
| 2-1-505 | N-Biph | N-Biph | H | H | b7 | H |
| 2-1-506 | N-Biph | N-Biph | t-bu | H | b7 | H |
| 2-1-507 | N-Biph | N-Biph | methyl | H | b7 | H |
| 2-1-508 | N-Biph | N-Biph | b1 | H | b7 | H |
| 2-1-509 | N-Biph | N-Biph | b2 | H | b7 | H |
| 2-1-510 | N-Biph | N-Biph | b3 | H | b7 | H |
| 2-1-511 | N-Biph | N-Biph | H | H | b8 | H |
| 2-1-512 | N-Biph | N-Biph | t-bu | H | b8 | H |
| 2-1-513 | N-Biph | N-Biph | methyl | H | b8 | H |
| 2-1-514 | N-Biph | N-Biph | b1 | H | b8 | H |
| 2-1-515 | N-Biph | N-Biph | b2 | H | b8 | H |
| 2-1-516 | N-Biph | N-Biph | b3 | H | b8 | H |
| 2-1-517 | N-Biph | N-Biph | H | H | b9 | H |
| 2-1-518 | N-Biph | N-Biph | t-bu | H | b9 | H |
| 2-1-519 | N-Biph | N-Biph | methyl | H | b9 | H |
| 2-1-520 | N-Biph | N-Biph | b1 | H | b9 | H |
| 2-1-521 | N-Biph | N-Biph | b2 | H | b9 | H |
| 2-1-522 | N-Biph | N-Biph | b3 | H | b9 | H |
| 2-1-523 | N-Biph | N-Biph | H | H | b10 | H |
| 2-1-524 | N-Biph | N-Biph | t-bu | H | b10 | H |
| 2-1-525 | N-Biph | N-Biph | methyl | H | b10 | H |
| 2-1-526 | N-Biph | N-Biph | b1 | H | b10 | H |
| 2-1-527 | N-Biph | N-Biph | b2 | H | b10 | H |
| 2-1-528 | N-Biph | N-Biph | b3 | H | b10 | H |
| 2-1-529 | N-Biph | N-Biph | H | H | a1 | a1 |
| 2-1-530 | N-Biph | N-Biph | t-bu | H | a1 | a1 |
| 2-1-531 | N-Biph | N-Biph | methyl | H | a1 | a1 |
| 2-1-532 | N-Biph | N-Biph | b1 | H | a1 | a1 |
| 2-1-533 | N-Biph | N-Biph | b2 | H | a1 | a1 |
| 2-1-534 | N-Biph | N-Biph | b3 | H | a1 | a1 |
| 2-1-535 | N-Biph | N-Biph | H | H | a2 | a2 |
| 2-1-536 | N-Biph | N-Biph | t-bu | H | a2 | a2 |
| 2-1-537 | N-Biph | N-Biph | methyl | H | a2 | a2 |
| 2-1-538 | N-Biph | N-Biph | b1 | H | a2 | a2 |
| 2-1-539 | N-Biph | N-Biph | b2 | H | a2 | a2 |
| 2-1-540 | N-Biph | N-Biph | b3 | H | a2 | a2 |
| 2-1-541 | N-Biph | N-Biph | H | H | a3 | a3 |
| 2-1-542 | N-Biph | N-Biph | t-bu | H | a3 | a3 |
| 2-1-543 | N-Biph | N-Biph | methyl | H | a3 | a3 |
| 2-1-544 | N-Biph | N-Biph | b1 | H | a3 | a3 |
| 2-1-545 | N-Biph | N-Biph | b2 | H | a3 | a3 |
| 2-1-546 | N-Biph | N-Biph | b3 | H | a3 | a3 |
| 2-1-547 | N-Biph | N-Biph | H | H | b1 | b1 |
| 2-1-548 | N-Biph | N-Biph | t-bu | H | b1 | b1 |
| 2-1-549 | N-Biph | N-Biph | methyl | H | b1 | b1 |
| 2-1-550 | N-Biph | N-Biph | b1 | H | b1 | b1 |
| 2-1-551 | N-Biph | N-Biph | b2 | H | b1 | b1 |
| 2-1-552 | N-Biph | N-Biph | b3 | H | b1 | b1 |
| 2-1-553 | N-Biph | N-Biph | H | H | b2 | b2 |
| 2-1-554 | N-Biph | N-Biph | t-bu | H | b2 | b2 |
| 2-1-555 | N-Biph | N-Biph | methyl | H | b2 | b2 |
| 2-1-556 | N-Biph | N-Biph | b1 | H | b2 | b2 |
| 2-1-557 | N-Biph | N-Biph | b2 | H | b2 | b2 |
| 2-1-558 | N-Biph | N-Biph | b3 | H | b2 | b2 |
| 2-1-559 | N-Biph | N-Biph | H | H | b3 | b3 |
| 2-1-560 | N-Biph | N-Biph | t-bu | H | b3 | b3 |
| 2-1-561 | N-Biph | N-Biph | methyl | H | b3 | b3 |
| 2-1-562 | N-Biph | N-Biph | b1 | H | b3 | b3 |
| 2-1-563 | N-Biph | N-Biph | b2 | H | b3 | b3 |
| 2-1-564 | N-Biph | N-Biph | b3 | H | b3 | b3 |
| 2-1-565 | N-Biph | N-Biph | H | H | b5 | b5 |
| 2-1-566 | N-Biph | N-Biph | t-bu | H | b5 | b5 |
| 2-1-567 | N-Biph | N-Biph | methyl | H | b5 | b5 |
| 2-1-568 | N-Biph | N-Biph | b1 | H | b5 | b5 |
| 2-1-569 | N-Biph | N-Biph | b2 | H | b5 | b5 |
| 2-1-570 | N-Biph | N-Biph | b3 | H | b5 | b5 |
| 2-1-571 | N-Biph | N-Biph | H | H | b6 | b6 |
| 2-1-572 | N-Biph | N-Biph | t-bu | H | b6 | b6 |
| 2-1-573 | N-Biph | N-Biph | methyl | H | b6 | b6 |
| 2-1-574 | N-Biph | N-Biph | b1 | H | b6 | b6 |
| 2-1-575 | N-Biph | N-Biph | b2 | H | b6 | b6 |
| 2-1-576 | N-Biph | N-Biph | b3 | H | b6 | b6 |
| 2-1-577 | N-Biph | N-Biph | H | H | b7 | b7 |
| 2-1-578 | N-Biph | N-Biph | t-bu | H | b7 | b7 |
| 2-1-579 | N-Biph | N-Biph | methyl | H | b7 | b7 |
| 2-1-580 | N-Biph | N-Biph | b1 | H | b7 | b7 |
| 2-1-581 | N-Biph | N-Biph | b2 | H | b7 | b7 |
| 2-1-582 | N-Biph | N-Biph | b3 | H | b7 | b7 |
| 2-1-583 | O | N—Ph | H | H | H | H |
| 2-1-584 | O | N—Ph | t-bu | H | H | H |
| 2-1-585 | O | N—Ph | methyl | H | H | H |
| 2-1-586 | O | N—Ph | t-bu | t-bu | t-bu | t-bu |
| 2-1-587 | O | N—Ph | methyl | methyl | methyl | methyl |
| 2-1-588 | O | N—Ph | b1 | H | H | H |
| 2-1-589 | O | N—Ph | b2 | H | H | H |
| 2-1-590 | O | N—Ph | b3 | H | H | H |
| 2-1-591 | O | N—Ph | H | H | H | a1 |
| 2-1-592 | O | N—Ph | t-bu | H | H | a1 |
| 2-1-593 | O | N—Ph | methyl | H | H | a1 |
| 2-1-594 | O | N—Ph | b1 | H | H | a1 |
| 2-1-595 | O | N—Ph | b2 | H | H | a1 |
| 2-1-596 | O | N—Ph | b3 | H | H | a1 |
| 2-1-597 | O | N—Ph | H | H | H | a2 |
| 2-1-598 | O | N—Ph | t-bu | H | H | a2 |
| 2-1-599 | O | N—Ph | methyl | H | H | a2 |
| 2-1-600 | O | N—Ph | b1 | H | H | a2 |
| 2-1-601 | O | N—Ph | b2 | H | H | a2 |
| 2-1-602 | O | N—Ph | b3 | H | H | a2 |
| 2-1-603 | O | N—Ph | H | H | H | a3 |
| 2-1-604 | O | N—Ph | t-bu | H | H | a3 |
| 2-1-605 | O | N—Ph | methyl | H | H | a3 |
| 2-1-606 | O | N—Ph | b1 | H | H | a3 |
| 2-1-607 | O | N—Ph | b2 | H | H | a3 |
| 2-1-608 | O | N—Ph | b3 | H | H | a3 |
| 2-1-609 | O | N—Ph | H | H | H | a4 |
| 2-1-610 | O | N—Ph | t-bu | H | H | a4 |
| 2-1-611 | O | N—Ph | methyl | H | H | a4 |
| 2-1-612 | O | N—Ph | b1 | H | H | a4 |
| 2-1-613 | O | N—Ph | b2 | H | H | a4 |
| 2-1-614 | O | N—Ph | b3 | H | H | a4 |
| 2-1-615 | O | N—Ph | H | H | H | a5 |
| 2-1-616 | O | N—Ph | t-bu | H | H | a5 |
| 2-1-617 | O | N—Ph | methyl | H | H | a5 |
| 2-1-618 | O | N—Ph | b1 | H | H | a5 |
| 2-1-619 | O | N—Ph | b2 | H | H | a5 |
| 2-1-620 | O | N—Ph | b3 | H | H | a5 |
| 2-1-621 | O | N—Ph | H | H | H | a6 |
| 2-1-622 | O | N—Ph | t-bu | H | H | a6 |
| 2-1-623 | O | N—Ph | methyl | H | H | a6 |
| 2-1-624 | O | N—Ph | b1 | H | H | a6 |
| 2-1-625 | O | N—Ph | b2 | H | H | a6 |
| 2-1-626 | O | N—Ph | b3 | H | H | a6 |
| 2-1-627 | O | N—Ph | H | H | a1 | H |
| 2-1-628 | O | N—Ph | t-bu | H | a1 | H |
| 2-1-629 | O | N—Ph | methyl | H | a1 | H |
| 2-1-630 | O | N—Ph | b1 | H | a1 | H |
| 2-1-631 | O | N—Ph | b2 | H | a1 | H |
| 2-1-632 | O | N—Ph | b3 | H | a1 | H |
| 2-1-633 | O | N—Ph | H | H | a2 | H |
| 2-1-634 | O | N—Ph | t-bu | H | a2 | H |
| 2-1-635 | O | N—Ph | methyl | H | a2 | H |
| 2-1-636 | O | N—Ph | b1 | H | a2 | H |
| 2-1-637 | O | N—Ph | b2 | H | a2 | H |
| 2-1-638 | O | N—Ph | b3 | H | a2 | H |
| 2-1-639 | O | N—Ph | H | H | a3 | H |
| 2-1-640 | O | N—Ph | t-bu | H | a3 | H |
| 2-1-641 | O | N—Ph | methyl | H | a3 | H |
| 2-1-642 | O | N—Ph | b1 | H | a3 | H |
| 2-1-643 | O | N—Ph | b2 | H | a3 | H |
| 2-1-644 | O | N—Ph | b3 | H | a3 | H |
| 2-1-645 | O | N—Ph | H | H | a4 | H |
| 2-1-646 | O | N—Ph | t-bu | H | a4 | H |
| 2-1-647 | O | N—Ph | methyl | H | a4 | H |
| 2-1-648 | O | N—Ph | b1 | H | a4 | H |
| 2-1-649 | O | N—Ph | b2 | H | a4 | H |
| 2-1-650 | O | N—Ph | b3 | H | a4 | H |
| 2-1-651 | O | N—Ph | H | H | a5 | H |
| 2-1-652 | O | N—Ph | t-bu | H | a5 | H |
| 2-1-653 | O | N—Ph | methyl | H | a5 | H |
| 2-1-654 | O | N—Ph | b1 | H | a5 | H |
| 2-1-655 | O | N—Ph | b2 | H | a5 | H |
| 2-1-656 | O | N—Ph | b3 | H | a5 | H |
| 2-1-657 | O | N—Ph | H | H | a6 | H |
| 2-1-658 | O | N—Ph | t-bu | H | a6 | H |
| 2-1-659 | O | N—Ph | methyl | H | a6 | H |
| 2-1-660 | O | N—Ph | b1 | H | a6 | H |
| 2-1-661 | O | N—Ph | b2 | H | a6 | H |
| 2-1-662 | O | N—Ph | b3 | H | a6 | H |
| 2-1-663 | O | N—Ph | H | H | b1 | H |
| 2-1-664 | O | N—Ph | t-bu | H | b1 | H |
| 2-1-665 | O | N—Ph | methyl | H | b1 | H |
| 2-1-666 | O | N—Ph | b1 | H | b1 | H |
| 2-1-667 | O | N—Ph | b2 | H | b1 | H |
| 2-1-668 | O | N—Ph | b3 | H | b1 | H |
| 2-1-669 | O | N—Ph | H | H | b2 | H |
| 2-1-670 | O | N—Ph | t-bu | H | b2 | H |
| 2-1-671 | O | N—Ph | methyl | H | b2 | H |
| 2-1-672 | O | N—Ph | b1 | H | b2 | H |
| 2-1-673 | O | N—Ph | b2 | H | b2 | H |
| 2-1-674 | O | N—Ph | b3 | H | b2 | H |
| 2-1-675 | O | N—Ph | H | H | b3 | H |
| 2-1-676 | O | N—Ph | t-bu | H | b3 | H |
| 2-1-677 | O | N—Ph | methyl | H | b3 | H |
| 2-1-678 | O | N—Ph | b1 | H | b3 | H |
| 2-1-679 | O | N—Ph | b2 | H | b3 | H |
| 2-1-680 | O | N—Ph | b3 | H | b3 | H |
| 2-1-681 | O | N—Ph | H | H | b4 | H |
| 2-1-682 | O | N—Ph | t-bu | H | b4 | H |
| 2-1-683 | O | N—Ph | methyl | H | b4 | H |
| 2-1-684 | O | N—Ph | b1 | H | b4 | H |
| 2-1-685 | O | N—Ph | b2 | H | b4 | H |
| 2-1-686 | O | N—Ph | b3 | H | b4 | H |
| 2-1-687 | O | N—Ph | H | H | b5 | H |
| 2-1-688 | O | N—Ph | t-bu | H | b5 | H |
| 2-1-689 | O | N—Ph | methyl | H | b5 | H |
| 2-1-690 | O | N—Ph | b1 | H | b5 | H |
| 2-1-691 | O | N—Ph | b2 | H | b5 | H |
| 2-1-692 | O | N—Ph | b3 | H | b5 | H |
| 2-1-693 | O | N—Ph | H | H | b6 | H |
| 2-1-694 | O | N—Ph | t-bu | H | b6 | H |
| 2-1-695 | O | N—Ph | methyl | H | b6 | H |
| 2-1-696 | O | N—Ph | b1 | H | b6 | H |
| 2-1-697 | O | N—Ph | b2 | H | b6 | H |
| 2-1-698 | O | N—Ph | b3 | H | b6 | H |
| 2-1-699 | O | N—Ph | H | H | b7 | H |
| 2-1-700 | O | N—Ph | t-bu | H | b7 | H |
| 2-1-701 | O | N—Ph | methyl | H | b7 | H |
| 2-1-702 | O | N—Ph | b1 | H | b7 | H |
| 2-1-703 | O | N—Ph | b2 | H | b7 | H |
| 2-1-704 | O | N—Ph | b3 | H | b7 | H |
| 2-1-705 | O | N—Ph | H | H | b8 | H |
| 2-1-706 | O | N—Ph | t-bu | H | b8 | H |
| 2-1-707 | O | N—Ph | methyl | H | b8 | H |
| 2-1-708 | O | N—Ph | b1 | H | b8 | H |
| 2-1-709 | O | N—Ph | b2 | H | b8 | H |
| 2-1-710 | O | N—Ph | b3 | H | b8 | H |
| 2-1-711 | O | N—Ph | H | H | b9 | H |
| 2-1-712 | O | N—Ph | t-bu | H | b9 | H |
| 2-1-713 | O | N—Ph | methyl | H | b9 | H |
| 2-1-714 | O | N—Ph | b1 | H | b9 | H |
| 2-1-715 | O | N—Ph | b2 | H | b9 | H |
| 2-1-716 | O | N—Ph | b3 | H | b9 | H |
| 2-1-717 | O | N—Ph | H | H | b10 | H |
| 2-1-718 | O | N—Ph | t-bu | H | b10 | H |
| 2-1-719 | O | N—Ph | methyl | H | b10 | H |
| 2-1-720 | O | N—Ph | b1 | H | b10 | H |
| 2-1-721 | O | N—Ph | b2 | H | b10 | H |
| 2-1-722 | O | N—Ph | b3 | H | b10 | H |
| 2-1-723 | O | N—Ph | H | H | a1 | a1 |
| 2-1-724 | O | N—Ph | t-bu | H | a1 | a1 |
| 2-1-725 | O | N—Ph | methyl | H | a1 | a1 |
| 2-1-726 | O | N—Ph | b1 | H | a1 | a1 |
| 2-1-727 | O | N—Ph | b2 | H | a1 | a1 |
| 2-1-728 | O | N—Ph | b3 | H | a1 | a1 |
| 2-1-729 | O | N—Ph | H | H | a2 | a2 |
| 2-1-730 | O | N—Ph | t-bu | H | a2 | a2 |
| 2-1-731 | O | N—Ph | methyl | H | a2 | a2 |
| 2-1-732 | O | N—Ph | b1 | H | a2 | a2 |
| 2-1-733 | O | N—Ph | b2 | H | a2 | a2 |
| 2-1-734 | O | N—Ph | b3 | H | a2 | a2 |
| 2-1-735 | O | N—Ph | H | H | a3 | a3 |
| 2-1-736 | O | N—Ph | t-bu | H | a3 | a3 |
| 2-1-737 | O | N—Ph | methyl | H | a3 | a3 |
| 2-1-738 | O | N—Ph | b1 | H | a3 | a3 |
| 2-1-739 | O | N—Ph | b2 | H | a3 | a3 |
| 2-1-740 | O | N—Ph | b3 | H | a3 | a3 |
| 2-1-741 | O | N—Ph | H | H | b1 | b1 |
| 2-1-742 | O | N—Ph | t-bu | H | b1 | b1 |
| 2-1-743 | O | N—Ph | methyl | H | b1 | b1 |
| 2-1-744 | O | N—Ph | b1 | H | b1 | b1 |
| 2-1-745 | O | N—Ph | b2 | H | b1 | b1 |
| 2-1-746 | O | N—Ph | b3 | H | b1 | b1 |
| 2-1-747 | O | N—Ph | H | H | b2 | b2 |
| 2-1-748 | O | N—Ph | t-bu | H | b2 | b2 |
| 2-1-749 | O | N—Ph | methyl | H | b2 | b2 |
| 2-1-750 | O | N—Ph | b1 | H | b2 | b2 |
| 2-1-751 | O | N—Ph | b2 | H | b2 | b2 |
| 2-1-752 | O | N—Ph | b3 | H | b2 | b2 |
| 2-1-753 | O | N—Ph | H | H | b3 | b3 |
| 2-1-754 | O | N—Ph | t-bu | H | b3 | b3 |
| 2-1-755 | O | N—Ph | methyl | H | b3 | b3 |
| 2-1-756 | O | N—Ph | b1 | H | b3 | b3 |
| 2-1-757 | O | N—Ph | b2 | H | b3 | b3 |
| 2-1-758 | O | N—Ph | b3 | H | b3 | b3 |
| 2-1-759 | O | N—Ph | H | H | b5 | b5 |
| 2-1-760 | O | N—Ph | t-bu | H | b5 | b5 |
| 2-1-761 | O | N—Ph | methyl | H | b5 | b5 |
| 2-1-762 | O | N—Ph | b1 | H | b5 | b5 |
| 2-1-763 | O | N—Ph | b2 | H | b5 | b5 |
| 2-1-764 | O | N—Ph | b3 | H | b5 | b5 |
| 2-1-765 | O | N—Ph | H | H | b6 | b6 |
| 2-1-766 | O | N—Ph | t-bu | H | b6 | b6 |
| 2-1-767 | O | N—Ph | methyl | H | b6 | b6 |
| 2-1-768 | O | N—Ph | b1 | H | b6 | b6 |
| 2-1-769 | O | N—Ph | b2 | H | b6 | b6 |
| 2-1-770 | O | N—Ph | b3 | H | b6 | b6 |
| 2-1-771 | O | N—Ph | H | H | b7 | b7 |
| 2-1-772 | O | N—Ph | t-bu | H | b7 | b7 |
| 2-1-773 | O | N—Ph | methyl | H | b7 | b7 |
| 2-1-774 | O | N—Ph | b1 | H | b7 | b7 |
| 2-1-775 | O | N—Ph | b2 | H | b7 | b7 |
| 2-1-776 | O | N—Ph | b3 | H | b7 | b7 |
| 2-1-777 | S | N—Ph | H | H | H | H |
| 2-1-778 | S | N—Ph | t-bu | H | H | H |
| 2-1-779 | S | N—Ph | methyl | H | H | H |
| 2-1-780 | S | N—Ph | t-bu | t-bu | t-bu | t-bu |
| 2-1-781 | S | N—Ph | methyl | methyl | methyl | methyl |
| 2-1-782 | S | N—Ph | b1 | H | H | H |
| 2-1-783 | S | N—Ph | b2 | H | H | H |
| 2-1-784 | S | N—Ph | b3 | H | H | H |
| 2-1-785 | S | N—Ph | H | H | H | a1 |
| 2-1-786 | S | N—Ph | t-bu | H | H | a1 |
| 2-1-787 | S | N—Ph | methyl | H | H | a1 |
| 2-1-788 | S | N—Ph | b1 | H | H | a1 |
| 2-1-789 | S | N—Ph | b2 | H | H | a1 |
| 2-1-790 | S | N—Ph | b3 | H | H | a1 |
| 2-1-791 | S | N—Ph | H | H | H | a2 |
| 2-1-792 | S | N—Ph | t-bu | H | H | a2 |
| 2-1-793 | S | N—Ph | methyl | H | H | a2 |
| 2-1-794 | S | N—Ph | b1 | H | H | a2 |
| 2-1-795 | S | N—Ph | b2 | H | H | a2 |
| 2-1-796 | S | N—Ph | b3 | H | H | a2 |
| 2-1-797 | S | N—Ph | H | H | H | a3 |
| 2-1-798 | S | N—Ph | t-bu | H | H | a3 |
| 2-1-799 | S | N—Ph | methyl | H | H | a3 |
| 2-1-800 | S | N—Ph | b1 | H | H | a3 |
| 2-1-801 | S | N—Ph | b2 | H | H | a3 |
| 2-1-802 | S | N—Ph | b3 | H | H | a3 |
| 2-1-803 | S | N—Ph | H | H | H | a4 |
| 2-1-804 | S | N—Ph | t-bu | H | H | a4 |
| 2-1-805 | S | N—Ph | methyl | H | H | a4 |
| 2-1-806 | S | N—Ph | b1 | H | H | a4 |
| 2-1-807 | S | N—Ph | b2 | H | H | a4 |
| 2-1-808 | S | N—Ph | b3 | H | H | a4 |
| 2-1-809 | S | N—Ph | H | H | H | a5 |
| 2-1-810 | S | N—Ph | t-bu | H | H | a5 |
| 2-1-811 | S | N—Ph | methyl | H | H | a5 |
| 2-1-812 | S | N—Ph | b1 | H | H | a5 |
| 2-1-813 | S | N—Ph | b2 | H | H | a5 |
| 2-1-814 | S | N—Ph | b3 | H | H | a5 |
| 2-1-815 | S | N—Ph | H | H | H | a6 |
| 2-1-816 | S | N—Ph | t-bu | H | H | a6 |
| 2-1-817 | S | N—Ph | methyl | H | H | a6 |
| 2-1-818 | S | N—Ph | b1 | H | H | a6 |
| 2-1-819 | S | N—Ph | b2 | H | H | a6 |
| 2-1-820 | S | N—Ph | b3 | H | H | a6 |
| 2-1-821 | S | N—Ph | H | H | a1 | H |
| 2-1-822 | S | N—Ph | t-bu | H | a1 | H |
| 2-1-823 | S | N—Ph | methyl | H | a1 | H |
| 2-1-824 | S | N—Ph | b1 | H | a1 | H |
| 2-1-825 | S | N—Ph | b2 | H | a1 | H |
| 2-1-826 | S | N—Ph | b3 | H | a1 | H |
| 2-1-827 | S | N—Ph | H | H | a2 | H |
| 2-1-828 | S | N—Ph | t-bu | H | a2 | H |
| 2-1-829 | S | N—Ph | methyl | H | a2 | H |
| 2-1-830 | S | N—Ph | b1 | H | a2 | H |
| 2-1-831 | S | N—Ph | b2 | H | a2 | H |
| 2-1-832 | S | N—Ph | b3 | H | a2 | H |
| 2-1-833 | S | N—Ph | H | H | a3 | H |
| 2-1-834 | S | N—Ph | t-bu | H | a3 | H |
| 2-1-835 | S | N—Ph | methyl | H | a3 | H |
| 2-1-836 | S | N—Ph | b1 | H | a3 | H |
| 2-1-837 | S | N—Ph | b2 | H | a3 | H |
| 2-1-838 | S | N—Ph | b3 | H | a3 | H |
| 2-1-839 | S | N—Ph | H | H | a4 | H |
| 2-1-840 | S | N—Ph | t-bu | H | a4 | H |
| 2-1-841 | S | N—Ph | methyl | H | a4 | H |
| 2-1-842 | S | N—Ph | b1 | H | a4 | H |
| 2-1-843 | S | N—Ph | b2 | H | a4 | H |
| 2-1-844 | S | N—Ph | b3 | H | a4 | H |
| 2-1-845 | S | N—Ph | H | H | a5 | H |
| 2-1-846 | S | N—Ph | t-bu | H | a5 | H |
| 2-1-847 | S | N—Ph | methyl | H | a5 | H |
| 2-1-848 | S | N—Ph | b1 | H | a5 | H |
| 2-1-849 | S | N—Ph | b2 | H | a5 | H |
| 2-1-850 | S | N—Ph | b3 | H | a5 | H |
| 2-1-851 | S | N—Ph | H | H | a6 | H |
| 2-1-852 | S | N—Ph | t-bu | H | a6 | H |
| 2-1-853 | S | N—Ph | methyl | H | a6 | H |
| 2-1-854 | S | N—Ph | b1 | H | a6 | H |
| 2-1-855 | S | N—Ph | b2 | H | a6 | H |
| 2-1-856 | S | N—Ph | b3 | H | a6 | H |
| 2-1-857 | S | N—Ph | H | H | b1 | H |
| 2-1-858 | S | N—Ph | t-bu | H | b1 | H |
| 2-1-859 | S | N—Ph | methyl | H | b1 | H |
| 2-1-860 | S | N—Ph | b1 | H | b1 | H |
| 2-1-861 | S | N—Ph | b2 | H | b1 | H |
| 2-1-862 | S | N—Ph | b3 | H | b1 | H |
| 2-1-863 | S | N—Ph | H | H | b2 | H |
| 2-1-864 | S | N—Ph | t-bu | H | b2 | H |
| 2-1-865 | S | N—Ph | methyl | H | b2 | H |
| 2-1-866 | S | N—Ph | b1 | H | b2 | H |
| 2-1-867 | S | N—Ph | b2 | H | b2 | H |
| 2-1-868 | S | N—Ph | b3 | H | b2 | H |
| 2-1-869 | S | N—Ph | H | H | b3 | H |
| 2-1-870 | S | N—Ph | t-bu | H | b3 | H |
| 2-1-871 | S | N—Ph | methyl | H | b3 | H |
| 2-1-872 | S | N—Ph | b1 | H | b3 | H |
| 2-1-873 | S | N—Ph | b2 | H | b3 | H |
| 2-1-874 | S | N—Ph | b3 | H | b3 | H |
| 2-1-875 | S | N—Ph | H | H | b4 | H |
| 2-1-876 | S | N—Ph | t-bu | H | b4 | H |
| 2-1-877 | S | N—Ph | methyl | H | b4 | H |
| 2-1-878 | S | N—Ph | b1 | H | b4 | H |
| 2-1-879 | S | N—Ph | b2 | H | b4 | H |
| 2-1-880 | S | N—Ph | b3 | H | b4 | H |
| 2-1-881 | S | N—Ph | H | H | b5 | H |
| 2-1-882 | S | N—Ph | t-bu | H | b5 | H |
| 2-1-883 | S | N—Ph | methyl | H | b5 | H |
| 2-1-884 | S | N—Ph | b1 | H | b5 | H |
| 2-1-885 | S | N—Ph | b2 | H | b5 | H |
| 2-1-886 | S | N—Ph | b3 | H | b5 | H |
| 2-1-887 | S | N—Ph | H | H | b6 | H |
| 2-1-888 | S | N—Ph | t-bu | H | b6 | H |
| 2-1-889 | S | N—Ph | methyl | H | b6 | H |
| 2-1-890 | S | N—Ph | b1 | H | b6 | H |
| 2-1-891 | S | N—Ph | b2 | H | b6 | H |
| 2-1-892 | S | N—Ph | b3 | H | b6 | H |
| 2-1-893 | S | N—Ph | H | H | b7 | H |
| 2-1-894 | S | N—Ph | t-bu | H | b7 | H |
| 2-1-895 | S | N—Ph | methyl | H | b7 | H |
| 2-1-896 | S | N—Ph | b1 | H | b7 | H |
| 2-1-897 | S | N—Ph | b2 | H | b7 | H |
| 2-1-898 | S | N—Ph | b3 | H | b7 | H |
| 2-1-899 | S | N—Ph | H | H | b8 | H |
| 2-1-900 | S | N—Ph | t-bu | H | b8 | H |
| 2-1-901 | S | N—Ph | methyl | H | b8 | H |
| 2-1-902 | S | N—Ph | b1 | H | b8 | H |
| 2-1-903 | S | N—Ph | b2 | H | b8 | H |
| 2-1-904 | S | N—Ph | b3 | H | b8 | H |
| 2-1-905 | S | N—Ph | H | H | b9 | H |
| 2-1-906 | S | N—Ph | t-bu | H | b9 | H |
| 2-1-907 | S | N—Ph | methyl | H | b9 | H |
| 2-1-908 | S | N—Ph | b1 | H | b9 | H |
| 2-1-909 | S | N—Ph | b2 | H | b9 | H |
| 2-1-910 | S | N—Ph | b3 | H | b9 | H |
| 2-1-911 | S | N—Ph | H | H | b10 | H |
| 2-1-912 | S | N—Ph | t-bu | H | b10 | H |
| 2-1-913 | S | N—Ph | methyl | H | b10 | H |
| 2-1-914 | S | N—Ph | b1 | H | b10 | H |
| 2-1-915 | S | N—Ph | b2 | H | b10 | H |
| 2-1-916 | S | N—Ph | b3 | H | b10 | H |
| 2-1-917 | S | N—Ph | H | H | a1 | a1 |
| 2-1-918 | S | N—Ph | t-bu | H | a1 | a1 |
| 2-1-919 | S | N—Ph | methyl | H | a1 | a1 |
| 2-1-920 | S | N—Ph | b1 | H | a1 | a1 |
| 2-1-921 | S | N—Ph | b2 | H | a1 | a1 |
| 2-1-922 | S | N—Ph | b3 | H | a1 | a1 |
| 2-1-923 | S | N—Ph | H | H | a2 | a2 |
| 2-1-924 | S | N—Ph | t-bu | H | a2 | a2 |
| 2-1-925 | S | N—Ph | methyl | H | a2 | a2 |
| 2-1-926 | S | N—Ph | b1 | H | a2 | a2 |
| 2-1-927 | S | N—Ph | b2 | H | a2 | a2 |
| 2-1-928 | S | N—Ph | b3 | H | a2 | a2 |
| 2-1-929 | S | N—Ph | H | H | a3 | a3 |
| 2-1-930 | S | N—Ph | t-bu | H | a3 | a3 |
| 2-1-931 | S | N—Ph | methyl | H | a3 | a3 |
| 2-1-932 | S | N—Ph | b1 | H | a3 | a3 |
| 2-1-933 | S | N—Ph | b2 | H | a3 | a3 |
| 2-1-934 | S | N—Ph | b3 | H | a3 | a3 |
| 2-1-935 | S | N—Ph | H | H | b1 | b1 |
| 2-1-936 | S | N—Ph | t-bu | H | b1 | b1 |
| 2-1-937 | S | N—Ph | methyl | H | b1 | b1 |
| 2-1-938 | S | N—Ph | b1 | H | b1 | b1 |
| 2-1-939 | S | N—Ph | b2 | H | b1 | b1 |
| 2-1-940 | S | N—Ph | b3 | H | b1 | b1 |
| 2-1-941 | S | N—Ph | H | H | b2 | b2 |
| 2-1-942 | S | N—Ph | t-bu | H | b2 | b2 |
| 2-1-943 | S | N—Ph | methyl | H | b2 | b2 |
| 2-1-944 | S | N—Ph | b1 | H | b2 | b2 |
| 2-1-945 | S | N—Ph | b2 | H | b2 | b2 |
| 2-1-946 | S | N—Ph | b3 | H | b2 | b2 |
| 2-1-947 | S | N—Ph | H | H | b3 | b3 |
| 2-1-948 | S | N—Ph | t-bu | H | b3 | b3 |
| 2-1-949 | S | N—Ph | methyl | H | b3 | b3 |
| 2-1-950 | S | N—Ph | b1 | H | b3 | b3 |
| 2-1-951 | S | N—Ph | b2 | H | b3 | b3 |
| 2-1-952 | S | N—Ph | b3 | H | b3 | b3 |
| 2-1-953 | S | N—Ph | H | H | b5 | b5 |
| 2-1-954 | S | N—Ph | t-bu | H | b5 | b5 |
| 2-1-955 | S | N—Ph | methyl | H | b5 | b5 |
| 2-1-956 | S | N—Ph | b1 | H | b5 | b5 |
| 2-1-957 | S | N—Ph | b2 | H | b5 | b5 |
| 2-1-958 | S | N—Ph | b3 | H | b5 | b5 |
| 2-1-959 | S | N—Ph | H | H | b6 | b6 |
| 2-1-960 | S | N—Ph | t-bu | H | b6 | b6 |
| 2-1-961 | S | N—Ph | methyl | H | b6 | b6 |
| 2-1-962 | S | N—Ph | b1 | H | b6 | b6 |
| 2-1-963 | S | N—Ph | b2 | H | b6 | b6 |
| 2-1-964 | S | N—Ph | b3 | H | b6 | b6 |
| 2-1-965 | S | N—Ph | H | H | b7 | b7 |
| 2-1-966 | S | N—Ph | t-bu | H | b7 | b7 |
| 2-1-967 | S | N—Ph | methyl | H | b7 | b7 |
| 2-1-968 | S | N—Ph | b1 | H | b7 | b7 |
| 2-1-969 | S | N—Ph | b2 | H | b7 | b7 |
| 2-1-970 | S | N—Ph | b3 | H | b7 | b7 |

### Group 2-2

The compounds are represented by the formula 2-2 below, wherein CY₂ and CY₃ are each a benzene group, X₁ is B, and Y1, Y2, R1a, R1c, (R2)a2, (R3)a3 are each shown in the table 2 below,

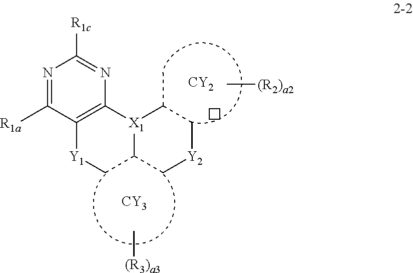

TABLE 2

| Compound No. | Y1 | Y2 | R1a | R1c | (R2)a2 | (R3)a3 |
|---|---|---|---|---|---|---|
| 2-2-001 | N—Ph | N—Ph | H | H | H | H |
| 2-2-002 | N—Ph | N—Ph | t-bu | t-bu | H | H |
| 2-2-003 | N—Ph | N—Ph | methyl | methyl | H | H |
| 2-2-004 | N—Ph | N—Ph | t-bu | t-bu | t-bu | t-bu |
| 2-2-005 | N—Ph | N—Ph | methyl | methyl | methyl | methyl |
| 2-2-006 | N—Ph | N—Ph | b1 | b1 | H | H |
| 2-2-007 | N—Ph | N—Ph | b2 | b2 | H | H |
| 2-2-008 | N—Ph | N—Ph | b3 | b3 | H | H |
| 2-2-009 | N—Ph | N—Ph | H | H | H | a1 |
| 2-2-010 | N—Ph | N—Ph | t-bu | t-bu | H | a1 |
| 2-2-011 | N—Ph | N—Ph | methyl | methyl | H | a1 |
| 2-2-012 | N—Ph | N—Ph | b1 | b1 | H | a1 |
| 2-2-013 | N—Ph | N—Ph | b2 | b2 | H | a1 |
| 2-2-014 | N—Ph | N—Ph | b3 | b3 | H | a1 |
| 2-2-015 | N—Ph | N—Ph | H | H | H | a2 |
| 2-2-016 | N—Ph | N—Ph | t-bu | t-bu | H | a2 |
| 2-2-017 | N—Ph | N—Ph | methyl | methyl | H | a2 |
| 2-2-018 | N—Ph | N—Ph | b1 | b1 | H | a2 |
| 2-2-019 | N—Ph | N—Ph | b2 | b2 | H | a2 |
| 2-2-020 | N—Ph | N—Ph | b3 | b3 | H | a2 |
| 2-2-021 | N—Ph | N—Ph | H | H | H | a3 |
| 2-2-022 | N—Ph | N—Ph | t-bu | t-bu | H | a3 |
| 2-2-023 | N—Ph | N—Ph | methyl | methyl | H | a3 |
| 2-2-024 | N—Ph | N—Ph | b1 | b1 | H | a3 |
| 2-2-025 | N—Ph | N—Ph | b2 | b2 | H | a3 |
| 2-2-026 | N—Ph | N—Ph | b3 | b3 | H | a3 |
| 2-2-027 | N—Ph | N—Ph | H | H | H | a4 |
| 2-2-028 | N—Ph | N—Ph | t-bu | t-bu | H | a4 |
| 2-2-029 | N—Ph | N—Ph | methyl | methyl | H | a4 |
| 2-2-030 | N—Ph | N—Ph | b1 | b1 | H | a4 |
| 2-2-031 | N—Ph | N—Ph | b2 | b2 | H | a4 |
| 2-2-032 | N—Ph | N—Ph | b3 | b3 | H | a4 |
| 2-2-033 | N—Ph | N—Ph | H | H | H | a5 |
| 2-2-034 | N—Ph | N—Ph | t-bu | t-bu | H | a5 |
| 2-2-035 | N—Ph | N—Ph | methyl | methyl | H | a5 |
| 2-2-036 | N—Ph | N—Ph | b1 | b1 | H | a5 |
| 2-2-037 | N—Ph | N—Ph | b2 | b2 | H | a5 |
| 2-2-038 | N—Ph | N—Ph | b3 | b3 | H | a5 |
| 2-2-039 | N—Ph | N—Ph | H | H | H | a6 |
| 2-2-040 | N—Ph | N—Ph | t-bu | t-bu | H | a6 |
| 2-2-041 | N—Ph | N—Ph | methyl | methyl | H | a6 |
| 2-2-042 | N—Ph | N—Ph | b1 | b1 | H | a6 |
| 2-2-043 | N—Ph | N—Ph | b2 | b2 | H | a6 |
| 2-2-044 | N—Ph | N—Ph | b3 | b3 | H | a6 |
| 2-2-045 | N—Ph | N—Ph | H | H | a1 | H |
| 2-2-046 | N—Ph | N—Ph | t-bu | t-bu | a1 | H |
| 2-2-047 | N—Ph | N—Ph | methyl | methyl | a1 | H |
| 2-2-048 | N—Ph | N—Ph | b1 | b1 | a1 | H |
| 2-2-049 | N—Ph | N—Ph | b2 | b2 | a1 | H |
| 2-2-050 | N—Ph | N—Ph | b3 | b3 | a1 | H |
| 2-2-051 | N—Ph | N—Ph | H | H | a2 | H |
| 2-2-052 | N—Ph | N—Ph | t-bu | t-bu | a2 | H |
| 2-2-053 | N—Ph | N—Ph | methyl | methyl | a2 | H |
| 2-2-054 | N—Ph | N—Ph | b1 | b1 | a2 | H |
| 2-2-055 | N—Ph | N—Ph | b2 | b2 | a2 | H |
| 2-2-056 | N—Ph | N—Ph | b3 | b3 | a2 | H |
| 2-2-057 | N—Ph | N—Ph | H | H | a3 | H |
| 2-2-058 | N—Ph | N—Ph | t-bu | t-bu | a3 | H |
| 2-2-059 | N—Ph | N—Ph | methyl | methyl | a3 | H |
| 2-2-060 | N—Ph | N—Ph | b1 | b1 | a3 | H |
| 2-2-061 | N—Ph | N—Ph | b2 | b2 | a3 | H |
| 2-2-062 | N—Ph | N—Ph | b3 | b3 | a3 | H |
| 2-2-063 | N—Ph | N—Ph | H | H | a4 | H |
| 2-2-064 | N—Ph | N—Ph | t-bu | t-bu | a4 | H |
| 2-2-065 | N—Ph | N—Ph | methyl | methyl | a4 | H |
| 2-2-066 | N—Ph | N—Ph | b1 | b1 | a4 | H |
| 2-2-067 | N—Ph | N—Ph | b2 | b2 | a4 | H |
| 2-2-068 | N—Ph | N—Ph | b3 | b3 | a4 | H |
| 2-2-069 | N—Ph | N—Ph | H | H | a5 | H |
| 2-2-070 | N—Ph | N—Ph | t-bu | t-bu | a5 | H |
| 2-2-071 | N—Ph | N—Ph | methyl | methyl | a5 | H |
| 2-2-072 | N—Ph | N—Ph | b1 | b1 | a5 | H |
| 2-2-073 | N—Ph | N—Ph | b2 | b2 | a5 | H |
| 2-2-074 | N—Ph | N—Ph | b3 | b3 | a5 | H |
| 2-2-075 | N—Ph | N—Ph | H | H | a6 | H |
| 2-2-076 | N—Ph | N—Ph | t-bu | t-bu | a6 | H |
| 2-2-077 | N—Ph | N—Ph | methyl | methyl | a6 | H |
| 2-2-078 | N—Ph | N—Ph | b1 | b1 | a6 | H |
| 2-2-079 | N—Ph | N—Ph | b2 | b2 | a6 | H |
| 2-2-080 | N—Ph | N—Ph | b3 | b3 | a6 | H |
| 2-2-081 | N—Ph | N—Ph | H | H | b1 | H |
| 2-2-082 | N—Ph | N—Ph | t-bu | t-bu | b1 | H |
| 2-2-083 | N—Ph | N—Ph | methyl | methyl | b1 | H |
| 2-2-084 | N—Ph | N—Ph | b1 | b1 | b1 | H |
| 2-2-085 | N—Ph | N—Ph | b2 | b2 | b1 | H |
| 2-2-086 | N—Ph | N—Ph | b3 | b3 | b1 | H |
| 2-2-087 | N—Ph | N—Ph | H | H | b2 | H |
| 2-2-088 | N—Ph | N—Ph | t-bu | t-bu | b2 | H |
| 2-2-089 | N—Ph | N—Ph | methyl | methyl | b2 | H |
| 2-2-090 | N—Ph | N—Ph | b1 | b1 | b2 | H |
| 2-2-091 | N—Ph | N—Ph | b2 | b2 | b2 | H |
| 2-2-092 | N—Ph | N—Ph | b3 | b3 | b2 | H |
| 2-2-093 | N—Ph | N—Ph | H | H | b3 | H |
| 2-2-094 | N—Ph | N—Ph | t-bu | t-bu | b3 | H |
| 2-2-095 | N—Ph | N—Ph | methyl | methyl | b3 | H |
| 2-2-096 | N—Ph | N—Ph | b1 | b1 | b3 | H |
| 2-2-097 | N—Ph | N—Ph | b2 | b2 | b3 | H |
| 2-2-098 | N—Ph | N—Ph | b3 | b3 | b3 | H |
| 2-2-099 | N—Ph | N—Ph | H | H | b4 | H |
| 2-2-100 | N—Ph | N—Ph | t-bu | t-bu | b4 | H |
| 2-2-101 | N—Ph | N—Ph | methyl | methyl | b4 | H |
| 2-2-102 | N—Ph | N—Ph | b1 | b1 | b4 | H |
| 2-2-103 | N—Ph | N—Ph | b2 | b2 | b4 | H |
| 2-2-104 | N—Ph | N—Ph | b3 | b3 | b4 | H |
| 2-2-105 | N—Ph | N—Ph | H | H | b5 | H |
| 2-2-106 | N—Ph | N—Ph | t-bu | t-bu | b5 | H |
| 2-2-107 | N—Ph | N—Ph | methyl | methyl | b5 | H |
| 2-2-108 | N—Ph | N—Ph | b1 | b1 | b5 | H |
| 2-2-109 | N—Ph | N—Ph | b2 | b2 | b5 | H |
| 2-2-110 | N—Ph | N—Ph | b3 | b3 | b5 | H |
| 2-2-111 | N—Ph | N—Ph | H | H | b6 | H |
| 2-2-112 | N—Ph | N—Ph | t-bu | t-bu | b6 | H |
| 2-2-113 | N—Ph | N—Ph | methyl | methyl | b6 | H |
| 2-2-114 | N—Ph | N—Ph | b1 | b1 | b6 | H |
| 2-2-115 | N—Ph | N—Ph | b2 | b2 | b6 | H |
| 2-2-116 | N—Ph | N—Ph | b3 | b3 | b6 | H |
| 2-2-117 | N—Ph | N—Ph | H | H | b7 | H |
| 2-2-118 | N—Ph | N—Ph | t-bu | t-bu | b7 | H |
| 2-2-119 | N—Ph | N—Ph | methyl | methyl | b7 | H |
| 2-2-120 | N—Ph | N—Ph | b1 | b1 | b7 | H |
| 2-2-121 | N—Ph | N—Ph | b2 | b2 | b7 | H |
| 2-2-122 | N—Ph | N—Ph | b3 | b3 | b7 | H |
| 2-2-123 | N—Ph | N—Ph | H | H | b8 | H |
| 2-2-124 | N—Ph | N—Ph | t-bu | t-bu | b8 | H |
| 2-2-125 | N—Ph | N—Ph | methyl | methyl | b8 | H |
| 2-2-126 | N—Ph | N—Ph | b1 | b1 | b8 | H |
| 2-2-127 | N—Ph | N—Ph | b2 | b2 | b8 | H |
| 2-2-128 | N—Ph | N—Ph | b3 | b3 | b8 | H |
| 2-2-129 | N—Ph | N—Ph | H | H | b9 | H |
| 2-2-130 | N—Ph | N—Ph | t-bu | t-bu | b9 | H |
| 2-2-131 | N—Ph | N—Ph | methyl | methyl | b9 | H |
| 2-2-132 | N—Ph | N—Ph | b1 | b1 | b9 | H |
| 2-2-133 | N—Ph | N—Ph | b2 | b2 | b9 | H |
| 2-2-134 | N—Ph | N—Ph | b3 | b3 | b9 | H |
| 2-2-135 | N—Ph | N—Ph | H | H | b10 | H |
| 2-2-136 | N—Ph | N—Ph | t-bu | t-bu | b10 | H |
| 2-2-137 | N—Ph | N—Ph | methyl | methyl | b10 | H |
| 2-2-138 | N—Ph | N—Ph | b1 | b1 | b10 | H |
| 2-2-139 | N—Ph | N—Ph | b2 | b2 | b10 | H |
| 2-2-140 | N—Ph | N—Ph | b3 | b3 | b10 | H |
| 2-2-141 | N—Ph | N—Ph | H | H | a1 | a1 |
| 2-2-142 | N—Ph | N—Ph | t-bu | t-bu | a1 | a1 |
| 2-2-143 | N—Ph | N—Ph | methyl | methyl | a1 | a1 |
| 2-2-144 | N—Ph | N—Ph | b1 | b1 | a1 | a1 |
| 2-2-145 | N—Ph | N—Ph | b2 | b2 | a1 | a1 |
| 2-2-146 | N—Ph | N—Ph | b3 | b3 | a1 | a1 |
| 2-2-147 | N—Ph | N—Ph | H | H | a2 | a2 |
| 2-2-148 | N—Ph | N—Ph | t-bu | t-bu | a2 | a2 |
| 2-2-149 | N—Ph | N—Ph | methyl | methyl | a2 | a2 |
| 2-2-150 | N—Ph | N—Ph | b1 | b1 | a2 | a2 |
| 2-2-151 | N—Ph | N—Ph | b2 | b2 | a2 | a2 |
| 2-2-152 | N—Ph | N—Ph | b3 | b3 | a2 | a2 |
| 2-2-153 | N—Ph | N—Ph | H | H | a3 | a3 |
| 2-2-154 | N—Ph | N—Ph | t-bu | t-bu | a3 | a3 |
| 2-2-155 | N—Ph | N—Ph | methyl | methyl | a3 | a3 |
| 2-2-156 | N—Ph | N—Ph | b1 | b1 | a3 | a3 |
| 2-2-157 | N—Ph | N—Ph | b2 | b2 | a3 | a3 |
| 2-2-158 | N—Ph | N—Ph | b3 | b3 | a3 | a3 |
| 2-2-159 | N—Ph | N—Ph | H | H | b1 | b1 |
| 2-2-160 | N—Ph | N—Ph | t-bu | t-bu | b1 | b1 |
| 2-2-161 | N—Ph | N—Ph | methyl | methyl | b1 | b1 |
| 2-2-162 | N—Ph | N—Ph | b1 | b1 | b1 | b1 |
| 2-2-163 | N—Ph | N—Ph | b2 | b2 | b1 | b1 |
| 2-2-164 | N—Ph | N—Ph | b3 | b3 | b1 | b1 |
| 2-2-165 | N—Ph | N—Ph | H | H | b2 | b2 |
| 2-2-166 | N—Ph | N—Ph | t-bu | t-bu | b2 | b2 |
| 2-2-167 | N—Ph | N—Ph | methyl | methyl | b2 | b2 |
| 2-2-168 | N—Ph | N—Ph | b1 | b1 | b2 | b2 |
| 2-2-169 | N—Ph | N—Ph | b2 | b2 | b2 | b2 |
| 2-2-170 | N—Ph | N—Ph | b3 | b3 | b2 | b2 |
| 2-2-171 | N—Ph | N—Ph | H | H | b3 | b3 |
| 2-2-172 | N—Ph | N—Ph | t-bu | t-bu | b3 | b3 |
| 2-2-173 | N—Ph | N—Ph | methyl | methyl | b3 | b3 |
| 2-2-174 | N—Ph | N—Ph | b1 | b1 | b3 | b3 |
| 2-2-175 | N—Ph | N—Ph | b2 | b2 | b3 | b3 |
| 2-2-176 | N—Ph | N—Ph | b3 | b3 | b3 | b3 |
| 2-2-177 | N—Ph | N—Ph | H | H | b5 | b5 |
| 2-2-178 | N—Ph | N—Ph | t-bu | t-bu | b5 | b5 |
| 2-2-179 | N—Ph | N—Ph | methyl | methyl | b5 | b5 |
| 2-2-180 | N—Ph | N—Ph | b1 | b1 | b5 | b5 |
| 2-2-181 | N—Ph | N—Ph | b2 | b2 | b5 | b5 |
| 2-2-182 | N—Ph | N—Ph | b3 | b3 | b5 | b5 |
| 2-2-183 | N—Ph | N—Ph | H | H | b6 | b6 |
| 2-2-184 | N—Ph | N—Ph | t-bu | t-bu | b6 | b6 |
| 2-2-185 | N—Ph | N—Ph | methyl | methyl | b6 | b6 |
| 2-2-186 | N—Ph | N—Ph | b1 | b1 | b6 | b6 |
| 2-2-187 | N—Ph | N—Ph | b2 | b2 | b6 | b6 |
| 2-2-188 | N—Ph | N—Ph | b3 | b3 | b6 | b6 |
| 2-2-189 | N—Ph | N—Ph | H | H | b7 | b7 |
| 2-2-190 | N—Ph | N—Ph | t-bu | t-bu | b7 | b7 |
| 2-2-191 | N—Ph | N—Ph | methyl | methyl | b7 | b7 |
| 2-2-192 | N—Ph | N—Ph | b1 | b1 | b7 | b7 |
| 2-2-193 | N—Ph | N—Ph | b2 | b2 | b7 | b7 |
| 2-2-194 | N—Ph | N—Ph | b3 | b3 | b7 | b7 |
| 2-2-195 | N-tolyl | N-tolyl | H | H | H | H |
| 2-2-196 | N-tolyl | N-tolyl | t-bu | t-bu | H | H |
| 2-2-197 | N-tolyl | N-tolyl | methyl | methyl | H | H |
| 2-2-198 | N-tolyl | N-tolyl | t-bu | t-bu | t-bu | t-bu |
| 2-2-199 | N-tolyl | N-tolyl | methyl | methyl | methyl | methyl |
| 2-2-200 | N-tolyl | N-tolyl | b1 | b1 | H | H |
| 2-2-201 | N-tolyl | N-tolyl | b2 | b2 | H | H |
| 2-2-202 | N-tolyl | N-tolyl | b3 | b3 | H | H |
| 2-2-203 | N-tolyl | N-tolyl | H | H | H | a1 |
| 2-2-204 | N-tolyl | N-tolyl | t-bu | t-bu | H | a1 |
| 2-2-205 | N-tolyl | N-tolyl | methyl | methyl | H | a1 |
| 2-2-206 | N-tolyl | N-tolyl | b1 | b1 | H | a1 |
| 2-2-207 | N-tolyl | N-tolyl | b2 | b2 | H | a1 |
| 2-2-208 | N-tolyl | N-tolyl | b3 | b3 | H | a1 |
| 2-2-209 | N-tolyl | N-tolyl | H | H | H | a2 |
| 2-2-210 | N-tolyl | N-tolyl | t-bu | t-bu | H | a2 |
| 2-2-211 | N-tolyl | N-tolyl | methyl | methyl | H | a2 |
| 2-2-212 | N-tolyl | N-tolyl | b1 | b1 | H | a2 |
| 2-2-213 | N-tolyl | N-tolyl | b2 | b2 | H | a2 |
| 2-2-214 | N-tolyl | N-tolyl | b3 | b3 | H | a2 |
| 2-2-215 | N-tolyl | N-tolyl | H | H | H | a3 |
| 2-2-216 | N-tolyl | N-tolyl | t-bu | t-bu | H | a3 |
| 2-2-217 | N-tolyl | N-tolyl | methyl | methyl | H | a3 |
| 2-2-218 | N-tolyl | N-tolyl | b1 | b1 | H | a3 |
| 2-2-219 | N-tolyl | N-tolyl | b2 | b2 | H | a3 |
| 2-2-220 | N-tolyl | N-tolyl | b3 | b3 | H | a3 |
| 2-2-221 | N-tolyl | N-tolyl | H | H | H | a4 |
| 2-2-222 | N-tolyl | N-tolyl | t-bu | t-bu | H | a4 |
| 2-2-223 | N-tolyl | N-tolyl | methyl | methyl | H | a4 |
| 2-2-224 | N-tolyl | N-tolyl | b1 | b1 | H | a4 |
| 2-2-225 | N-tolyl | N-tolyl | b2 | b2 | H | a4 |
| 2-2-226 | N-tolyl | N-tolyl | b3 | b3 | H | a4 |
| 2-2-227 | N-tolyl | N-tolyl | H | H | H | a5 |
| 2-2-228 | N-tolyl | N-tolyl | t-bu | t-bu | H | a5 |
| 2-2-229 | N-tolyl | N-tolyl | methyl | methyl | H | a5 |
| 2-2-230 | N-tolyl | N-tolyl | b1 | b1 | H | a5 |
| 2-2-231 | N-tolyl | N-tolyl | b2 | b2 | H | a5 |
| 2-2-232 | N-tolyl | N-tolyl | b3 | b3 | H | a5 |
| 2-2-233 | N-tolyl | N-tolyl | H | H | H | a6 |
| 2-2-234 | N-tolyl | N-tolyl | t-bu | t-bu | H | a6 |
| 2-2-235 | N-tolyl | N-tolyl | methyl | methyl | H | a6 |
| 2-2-236 | N-tolyl | N-tolyl | b1 | b1 | H | a6 |
| 2-2-237 | N-tolyl | N-tolyl | b2 | b2 | H | a6 |
| 2-2-238 | N-tolyl | N-tolyl | b3 | b3 | H | a6 |
| 2-2-239 | N-tolyl | N-tolyl | H | H | a1 | H |
| 2-2-240 | N-tolyl | N-tolyl | t-bu | t-bu | a1 | H |
| 2-2-241 | N-tolyl | N-tolyl | methyl | methyl | a1 | H |
| 2-2-242 | N-tolyl | N-tolyl | b1 | b1 | a1 | H |
| 2-2-243 | N-tolyl | N-tolyl | b2 | b2 | a1 | H |
| 2-2-244 | N-tolyl | N-tolyl | b3 | b3 | a1 | H |
| 2-2-245 | N-tolyl | N-tolyl | H | H | a2 | H |
| 2-2-246 | N-tolyl | N-tolyl | t-bu | t-bu | a2 | H |
| 2-2-247 | N-tolyl | N-tolyl | methyl | methyl | a2 | H |
| 2-2-248 | N-tolyl | N-tolyl | b1 | b1 | a2 | H |
| 2-2-249 | N-tolyl | N-tolyl | b2 | b2 | a2 | H |
| 2-2-250 | N-tolyl | N-tolyl | b3 | b3 | a2 | H |
| 2-2-251 | N-tolyl | N-tolyl | H | H | a3 | H |
| 2-2-252 | N-tolyl | N-tolyl | t-bu | t-bu | a3 | H |
| 2-2-253 | N-tolyl | N-tolyl | methyl | methyl | a3 | H |
| 2-2-254 | N-tolyl | N-tolyl | b1 | b1 | a3 | H |
| 2-2-255 | N-tolyl | N-tolyl | b2 | b2 | a3 | H |
| 2-2-256 | N-tolyl | N-tolyl | b3 | b3 | a3 | H |
| 2-2-257 | N-tolyl | N-tolyl | H | H | a4 | H |
| 2-2-258 | N-tolyl | N-tolyl | t-bu | t-bu | a4 | H |
| 2-2-259 | N-tolyl | N-tolyl | methyl | methyl | a4 | H |
| 2-2-260 | N-tolyl | N-tolyl | b1 | b1 | a4 | H |
| 2-2-261 | N-tolyl | N-tolyl | b2 | b2 | a4 | H |
| 2-2-262 | N-tolyl | N-tolyl | b3 | b3 | a4 | H |
| 2-2-263 | N-tolyl | N-tolyl | H | H | a5 | H |
| 2-2-264 | N-tolyl | N-tolyl | t-bu | t-bu | a5 | H |
| 2-2-265 | N-tolyl | N-tolyl | methyl | methyl | a5 | H |
| 2-2-266 | N-tolyl | N-tolyl | b1 | b1 | a5 | H |
| 2-2-267 | N-tolyl | N-tolyl | b2 | b2 | a5 | H |
| 2-2-268 | N-tolyl | N-tolyl | b3 | b3 | a5 | H |
| 2-2-269 | N-tolyl | N-tolyl | H | H | a6 | H |
| 2-2-270 | N-tolyl | N-tolyl | t-bu | t-bu | a6 | H |
| 2-2-271 | N-tolyl | N-tolyl | methyl | methyl | a6 | H |
| 2-2-272 | N-tolyl | N-tolyl | b1 | b1 | a6 | H |
| 2-2-273 | N-tolyl | N-tolyl | b2 | b2 | a6 | H |
| 2-2-274 | N-tolyl | N-tolyl | b3 | b3 | a6 | H |
| 2-2-275 | N-tolyl | N-tolyl | H | H | b1 | H |
| 2-2-276 | N-tolyl | N-tolyl | t-bu | t-bu | b1 | H |
| 2-2-277 | N-tolyl | N-tolyl | methyl | methyl | b1 | H |
| 2-2-278 | N-tolyl | N-tolyl | b1 | b1 | b1 | H |
| 2-2-279 | N-tolyl | N-tolyl | b2 | b2 | b1 | H |
| 2-2-280 | N-tolyl | N-tolyl | b3 | b3 | b1 | H |
| 2-2-281 | N-tolyl | N-tolyl | H | H | b2 | H |
| 2-2-282 | N-tolyl | N-tolyl | t-bu | t-bu | b2 | H |
| 2-2-283 | N-tolyl | N-tolyl | methyl | methyl | b2 | H |
| 2-2-284 | N-tolyl | N-tolyl | b1 | b1 | b2 | H |
| 2-2-285 | N-tolyl | N-tolyl | b2 | b2 | b2 | H |
| 2-2-286 | N-tolyl | N-tolyl | b3 | b3 | b2 | H |
| 2-2-287 | N-tolyl | N-tolyl | H | H | b3 | H |
| 2-2-288 | N-tolyl | N-tolyl | t-bu | t-bu | b3 | H |
| 2-2-289 | N-tolyl | N-tolyl | methyl | methyl | b3 | H |
| 2-2-290 | N-tolyl | N-tolyl | b1 | b1 | b3 | H |
| 2-2-291 | N-tolyl | N-tolyl | b2 | b2 | b3 | H |
| 2-2-292 | N-tolyl | N-tolyl | b3 | b3 | b3 | H |
| 2-2-293 | N-tolyl | N-tolyl | H | H | b4 | H |
| 2-2-294 | N-tolyl | N-tolyl | t-bu | t-bu | b4 | H |
| 2-2-295 | N-tolyl | N-tolyl | methyl | methyl | b4 | H |
| 2-2-296 | N-tolyl | N-tolyl | b1 | b1 | b4 | H |
| 2-2-297 | N-tolyl | N-tolyl | b2 | b2 | b4 | H |
| 2-2-298 | N-tolyl | N-tolyl | b3 | b3 | b4 | H |
| 2-2-299 | N-tolyl | N-tolyl | H | H | b5 | H |
| 2-2-300 | N-tolyl | N-tolyl | t-bu | t-bu | b5 | H |
| 2-2-301 | N-tolyl | N-tolyl | methyl | methyl | b5 | H |
| 2-2-302 | N-tolyl | N-tolyl | b1 | b1 | b5 | H |
| 2-2-303 | N-tolyl | N-tolyl | b2 | b2 | b5 | H |
| 2-2-304 | N-tolyl | N-tolyl | b3 | b3 | b5 | H |
| 2-2-305 | N-tolyl | N-tolyl | H | H | b6 | H |
| 2-2-306 | N-tolyl | N-tolyl | t-bu | t-bu | b6 | H |
| 2-2-307 | N-tolyl | N-tolyl | methyl | methyl | b6 | H |
| 2-2-308 | N-tolyl | N-tolyl | b1 | b1 | b6 | H |
| 2-2-309 | N-tolyl | N-tolyl | b2 | b2 | b6 | H |
| 2-2-310 | N-tolyl | N-tolyl | b3 | b3 | b6 | H |
| 2-2-311 | N-tolyl | N-tolyl | H | H | b7 | H |
| 2-2-312 | N-tolyl | N-tolyl | t-bu | t-bu | b7 | H |
| 2-2-313 | N-tolyl | N-tolyl | methyl | methyl | b7 | H |
| 2-2-314 | N-tolyl | N-tolyl | b1 | b1 | b7 | H |
| 2-2-315 | N-tolyl | N-tolyl | b2 | b2 | b7 | H |
| 2-2-316 | N-tolyl | N-tolyl | b3 | b3 | b7 | H |
| 2-2-317 | N-tolyl | N-tolyl | H | H | b8 | H |
| 2-2-318 | N-tolyl | N-tolyl | t-bu | t-bu | b8 | H |
| 2-2-319 | N-tolyl | N-tolyl | methyl | methyl | b8 | H |
| 2-2-320 | N-tolyl | N-tolyl | b1 | b1 | b8 | H |
| 2-2-321 | N-tolyl | N-tolyl | b2 | b2 | b8 | H |
| 2-2-322 | N-tolyl | N-tolyl | b3 | b3 | b8 | H |
| 2-2-323 | N-tolyl | N-tolyl | H | H | b9 | H |
| 2-2-324 | N-tolyl | N-tolyl | t-bu | t-bu | b9 | H |
| 2-2-325 | N-tolyl | N-tolyl | methyl | methyl | b9 | H |
| 2-2-326 | N-tolyl | N-tolyl | b1 | b1 | b9 | H |
| 2-2-327 | N-tolyl | N-tolyl | b2 | b2 | b9 | H |
| 2-2-328 | N-tolyl | N-tolyl | b3 | b3 | b9 | H |
| 2-2-329 | N-tolyl | N-tolyl | H | H | b10 | H |
| 2-2-330 | N-tolyl | N-tolyl | t-bu | t-bu | b10 | H |
| 2-2-331 | N-tolyl | N-tolyl | methyl | methyl | b10 | H |
| 2-2-332 | N-tolyl | N-tolyl | b1 | b1 | b10 | H |
| 2-2-333 | N-tolyl | N-tolyl | b2 | b2 | b10 | H |
| 2-2-334 | N-tolyl | N-tolyl | b3 | b3 | b10 | H |
| 2-2-335 | N-tolyl | N-tolyl | H | H | a1 | a1 |
| 2-2-336 | N-tolyl | N-tolyl | t-bu | t-bu | a1 | a1 |
| 2-2-337 | N-tolyl | N-tolyl | methyl | methyl | a1 | a1 |
| 2-2-338 | N-tolyl | N-tolyl | b1 | b1 | a1 | a1 |
| 2-2-339 | N-tolyl | N-tolyl | b2 | b2 | a1 | a1 |
| 2-2-340 | N-tolyl | N-tolyl | b3 | b3 | a1 | a1 |
| 2-2-341 | N-tolyl | N-tolyl | H | H | a2 | a2 |
| 2-2-342 | N-tolyl | N-tolyl | t-bu | t-bu | a2 | a2 |
| 2-2-343 | N-tolyl | N-tolyl | methyl | methyl | a2 | a2 |
| 2-2-344 | N-tolyl | N-tolyl | b1 | b1 | a2 | a2 |
| 2-2-345 | N-tolyl | N-tolyl | b2 | b2 | a2 | a2 |
| 2-2-346 | N-tolyl | N-tolyl | b3 | b3 | a2 | a2 |
| 2-2-347 | N-tolyl | N-tolyl | H | H | a3 | a3 |
| 2-2-348 | N-tolyl | N-tolyl | t-bu | t-bu | a3 | a3 |
| 2-2-349 | N-tolyl | N-tolyl | methyl | methyl | a3 | a3 |
| 2-2-350 | N-tolyl | N-tolyl | b1 | b1 | a3 | a3 |
| 2-2-351 | N-tolyl | N-tolyl | b2 | b2 | a3 | a3 |
| 2-2-352 | N-tolyl | N-tolyl | b3 | b3 | a3 | a3 |
| 2-2-353 | N-tolyl | N-tolyl | H | H | b1 | b1 |
| 2-2-354 | N-tolyl | N-tolyl | t-bu | t-bu | b1 | b1 |
| 2-2-355 | N-tolyl | N-tolyl | methyl | methyl | b1 | b1 |
| 2-2-356 | N-tolyl | N-tolyl | b1 | b1 | b1 | b1 |
| 2-2-357 | N-tolyl | N-tolyl | b2 | b2 | b1 | b1 |
| 2-2-358 | N-tolyl | N-tolyl | b3 | b3 | b1 | b1 |
| 2-2-359 | N-tolyl | N-tolyl | H | H | b2 | b2 |
| 2-2-360 | N-tolyl | N-tolyl | t-bu | t-bu | b2 | b2 |
| 2-2-361 | N-tolyl | N-tolyl | methyl | methyl | b2 | b2 |
| 2-2-362 | N-tolyl | N-tolyl | b1 | b1 | b2 | b2 |
| 2-2-363 | N-tolyl | N-tolyl | b2 | b2 | b2 | b2 |
| 2-2-364 | N-tolyl | N-tolyl | b3 | b3 | b2 | b2 |
| 2-2-365 | N-tolyl | N-tolyl | H | H | b3 | b3 |
| 2-2-366 | N-tolyl | N-tolyl | t-bu | t-bu | b3 | b3 |
| 2-2-367 | N-tolyl | N-tolyl | methyl | methyl | b3 | b3 |
| 2-2-368 | N-tolyl | N-tolyl | b1 | b1 | b3 | b3 |
| 2-2-369 | N-tolyl | N-tolyl | b2 | b2 | b3 | b3 |
| 2-2-370 | N-tolyl | N-tolyl | b3 | b3 | b3 | b3 |
| 2-2-371 | N-tolyl | N-tolyl | H | H | b5 | b5 |
| 2-2-372 | N-tolyl | N-tolyl | t-bu | t-bu | b5 | b5 |
| 2-2-373 | N-tolyl | N-tolyl | methyl | methyl | b5 | b5 |
| 2-2-374 | N-tolyl | N-tolyl | b1 | b1 | b5 | b5 |
| 2-2-375 | N-tolyl | N-tolyl | b2 | b2 | b5 | b5 |
| 2-2-376 | N-tolyl | N-tolyl | b3 | b3 | b5 | b5 |
| 2-2-377 | N-tolyl | N-tolyl | H | H | b6 | b6 |
| 2-2-378 | N-tolyl | N-tolyl | t-bu | t-bu | b6 | b6 |
| 2-2-379 | N-tolyl | N-tolyl | methyl | methyl | b6 | b6 |
| 2-2-380 | N-tolyl | N-tolyl | b1 | b1 | b6 | b6 |
| 2-2-381 | N-tolyl | N-tolyl | b2 | b2 | b6 | b6 |
| 2-2-382 | N-tolyl | N-tolyl | b3 | b3 | b6 | b6 |
| 2-2-383 | N-tolyl | N-tolyl | H | H | b7 | b7 |
| 2-2-384 | N-tolyl | N-tolyl | t-bu | t-bu | b7 | b7 |
| 2-2-385 | N-tolyl | N-tolyl | methyl | methyl | b7 | b7 |
| 2-2-386 | N-tolyl | N-tolyl | b1 | b1 | b7 | b7 |
| 2-2-387 | N-tolyl | N-tolyl | b2 | b2 | b7 | b7 |
| 2-2-388 | N-tolyl | N-tolyl | b3 | b3 | b7 | b7 |
| 2-2-389 | N-Biph | N-Biph | H | H | H | H |
| 2-2-390 | N-Biph | N-Biph | t-bu | t-bu | H | H |
| 2-2-391 | N-Biph | N-Biph | methyl | methyl | H | H |
| 2-2-392 | N-Biph | N-Biph | t-bu | t-bu | t-bu | t-bu |
| 2-2-393 | N-Biph | N-Biph | methyl | methyl | methyl | methyl |
| 2-2-394 | N-Biph | N-Biph | b1 | b1 | H | H |
| 2-2-395 | N-Biph | N-Biph | b2 | b2 | H | H |
| 2-2-396 | N-Biph | N-Biph | b3 | b3 | H | H |
| 2-2-397 | N-Biph | N-Biph | H | H | H | a1 |
| 2-2-398 | N-Biph | N-Biph | t-bu | t-bu | H | a1 |
| 2-2-399 | N-Biph | N-Biph | methyl | methyl | H | a1 |
| 2-2-400 | N-Biph | N-Biph | b1 | b1 | H | a1 |
| 2-2-401 | N-Biph | N-Biph | b2 | b2 | H | a1 |
| 2-2-402 | N-Biph | N-Biph | b3 | b3 | H | a1 |
| 2-2-403 | N-Biph | N-Biph | H | H | H | a2 |
| 2-2-404 | N-Biph | N-Biph | t-bu | t-bu | H | a2 |
| 2-2-405 | N-Biph | N-Biph | methyl | methyl | H | a2 |
| 2-2-406 | N-Biph | N-Biph | b1 | b1 | H | a2 |
| 2-2-407 | N-Biph | N-Biph | b2 | b2 | H | a2 |
| 2-2-408 | N-Biph | N-Biph | b3 | b3 | H | a2 |
| 2-2-409 | N-Biph | N-Biph | H | H | H | a3 |
| 2-2-410 | N-Biph | N-Biph | t-bu | t-bu | H | a3 |
| 2-2-411 | N-Biph | N-Biph | methyl | methyl | H | a3 |
| 2-2-412 | N-Biph | N-Biph | b1 | b1 | H | a3 |
| 2-2-413 | N-Biph | N-Biph | b2 | b2 | H | a3 |
| 2-2-414 | N-Biph | N-Biph | b3 | b3 | H | a3 |
| 2-2-415 | N-Biph | N-Biph | H | H | H | a4 |
| 2-2-416 | N-Biph | N-Biph | t-bu | t-bu | H | a4 |
| 2-2-417 | N-Biph | N-Biph | methyl | methyl | H | a4 |
| 2-2-418 | N-Biph | N-Biph | b1 | b1 | H | a4 |
| 2-2-419 | N-Biph | N-Biph | b2 | b2 | H | a4 |
| 2-2-420 | N-Biph | N-Biph | b3 | b3 | H | a4 |
| 2-2-421 | N-Biph | N-Biph | H | H | H | a5 |
| 2-2-422 | N-Biph | N-Biph | t-bu | t-bu | H | a5 |
| 2-2-423 | N-Biph | N-Biph | methyl | methyl | H | a5 |
| 2-2-424 | N-Biph | N-Biph | b1 | b1 | H | a5 |
| 2-2-425 | N-Biph | N-Biph | b2 | b2 | H | a5 |
| 2-2-426 | N-Biph | N-Biph | b3 | b3 | H | a5 |
| 2-2-427 | N-Biph | N-Biph | H | H | H | a6 |
| 2-2-428 | N-Biph | N-Biph | t-bu | t-bu | H | a6 |
| 2-2-429 | N-Biph | N-Biph | methyl | methyl | H | a6 |
| 2-2-430 | N-Biph | N-Biph | b1 | b1 | H | a6 |
| 2-2-431 | N-Biph | N-Biph | b2 | b2 | H | a6 |
| 2-2-432 | N-Biph | N-Biph | b3 | b3 | H | a6 |
| 2-2-433 | N-Biph | N-Biph | H | H | a1 | H |
| 2-2-434 | N-Biph | N-Biph | t-bu | t-bu | a1 | H |
| 2-2-435 | N-Biph | N-Biph | methyl | methyl | a1 | H |
| 2-2-436 | N-Biph | N-Biph | b1 | b1 | a1 | H |
| 2-2-437 | N-Biph | N-Biph | b2 | b2 | a1 | H |
| 2-2-438 | N-Biph | N-Biph | b3 | b3 | a1 | H |
| 2-2-439 | N-Biph | N-Biph | H | H | a2 | H |
| 2-2-440 | N-Biph | N-Biph | t-bu | t-bu | a2 | H |
| 2-2-441 | N-Biph | N-Biph | methyl | methyl | a2 | H |
| 2-2-442 | N-Biph | N-Biph | b1 | b1 | a2 | H |
| 2-2-443 | N-Biph | N-Biph | b2 | b2 | a2 | H |
| 2-2-444 | N-Biph | N-Biph | b3 | b3 | a2 | H |
| 2-2-445 | N-Biph | N-Biph | H | H | a3 | H |
| 2-2-446 | N-Biph | N-Biph | t-bu | t-bu | a3 | H |
| 2-2-447 | N-Biph | N-Biph | methyl | methyl | a3 | H |
| 2-2-448 | N-Biph | N-Biph | b1 | b1 | a3 | H |
| 2-2-449 | N-Biph | N-Biph | b2 | b2 | a3 | H |
| 2-2-450 | N-Biph | N-Biph | b3 | b3 | a3 | H |
| 2-2-451 | N-Biph | N-Biph | H | H | a4 | H |
| 2-2-452 | N-Biph | N-Biph | t-bu | t-bu | a4 | H |
| 2-2-453 | N-Biph | N-Biph | methyl | methyl | a4 | H |
| 2-2-454 | N-Biph | N-Biph | b1 | b1 | a4 | H |
| 2-2-455 | N-Biph | N-Biph | b2 | b2 | a4 | H |
| 2-2-456 | N-Biph | N-Biph | b3 | b3 | a4 | H |
| 2-2-457 | N-Biph | N-Biph | H | H | a5 | H |
| 2-2-458 | N-Biph | N-Biph | t-bu | t-bu | a5 | H |
| 2-2-459 | N-Biph | N-Biph | methyl | methyl | a5 | H |
| 2-2-460 | N-Biph | N-Biph | b1 | b1 | a5 | H |
| 2-2-461 | N-Biph | N-Biph | b2 | b2 | a5 | H |
| 2-2-462 | N-Biph | N-Biph | b3 | b3 | a5 | H |
| 2-2-463 | N-Biph | N-Biph | H | H | a6 | H |
| 2-2-464 | N-Biph | N-Biph | t-bu | t-bu | a6 | H |
| 2-2-465 | N-Biph | N-Biph | methyl | methyl | a6 | H |
| 2-2-466 | N-Biph | N-Biph | b1 | b1 | a6 | H |
| 2-2-467 | N-Biph | N-Biph | b2 | b2 | a6 | H |
| 2-2-468 | N-Biph | N-Biph | b3 | b3 | a6 | H |
| 2-2-469 | N-Biph | N-Biph | H | H | b1 | H |
| 2-2-470 | N-Biph | N-Biph | t-bu | t-bu | b1 | H |
| 2-2-471 | N-Biph | N-Biph | methyl | methyl | b1 | H |
| 2-2-472 | N-Biph | N-Biph | b1 | b1 | b1 | H |
| 2-2-473 | N-Biph | N-Biph | b2 | b2 | b1 | H |
| 2-2-474 | N-Biph | N-Biph | b3 | b3 | b1 | H |
| 2-2-475 | N-Biph | N-Biph | H | H | b2 | H |
| 2-2-476 | N-Biph | N-Biph | t-bu | t-bu | b2 | H |
| 2-2-477 | N-Biph | N-Biph | methyl | methyl | b2 | H |
| 2-2-478 | N-Biph | N-Biph | b1 | b1 | b2 | H |
| 2-2-479 | N-Biph | N-Biph | b2 | b2 | b2 | H |
| 2-2-480 | N-Biph | N-Biph | b3 | b3 | b2 | H |
| 2-2-481 | N-Biph | N-Biph | H | H | b3 | H |
| 2-2-482 | N-Biph | N-Biph | t-bu | t-bu | b3 | H |
| 2-2-483 | N-Biph | N-Biph | methyl | methyl | b3 | H |
| 2-2-484 | N-Biph | N-Biph | b1 | b1 | b3 | H |
| 2-2-485 | N-Biph | N-Biph | b2 | b2 | b3 | H |
| 2-2-486 | N-Biph | N-Biph | b3 | b3 | b3 | H |
| 2-2-487 | N-Biph | N-Biph | H | H | b4 | H |
| 2-2-488 | N-Biph | N-Biph | t-bu | t-bu | b4 | H |
| 2-2-489 | N-Biph | N-Biph | methyl | methyl | b4 | H |
| 2-2-490 | N-Biph | N-Biph | b1 | b1 | b4 | H |
| 2-2-491 | N-Biph | N-Biph | b2 | b2 | b4 | H |
| 2-2-492 | N-Biph | N-Biph | b3 | b3 | b4 | H |
| 2-2-493 | N-Biph | N-Biph | H | H | b5 | H |
| 2-2-494 | N-Biph | N-Biph | t-bu | t-bu | b5 | H |
| 2-2-495 | N-Biph | N-Biph | methyl | methyl | b5 | H |
| 2-2-496 | N-Biph | N-Biph | b1 | b1 | b5 | H |
| 2-2-497 | N-Biph | N-Biph | b2 | b2 | b5 | H |
| 2-2-498 | N-Biph | N-Biph | b3 | b3 | b5 | H |
| 2-2-499 | N-Biph | N-Biph | H | H | b6 | H |
| 2-2-500 | N-Biph | N-Biph | t-bu | t-bu | b6 | H |
| 2-2-501 | N-Biph | N-Biph | methyl | methyl | b6 | H |
| 2-2-502 | N-Biph | N-Biph | b1 | b1 | b6 | H |
| 2-2-503 | N-Biph | N-Biph | b2 | b2 | b6 | H |
| 2-2-504 | N-Biph | N-Biph | b3 | b3 | b6 | H |
| 2-2-505 | N-Biph | N-Biph | H | H | b7 | H |
| 2-2-506 | N-Biph | N-Biph | t-bu | t-bu | b7 | H |
| 2-2-507 | N-Biph | N-Biph | methyl | methyl | b7 | H |
| 2-2-508 | N-Biph | N-Biph | b1 | b1 | b7 | H |
| 2-2-509 | N-Biph | N-Biph | b2 | b2 | b7 | H |
| 2-2-510 | N-Biph | N-Biph | b3 | b3 | b7 | H |
| 2-2-511 | N-Biph | N-Biph | H | H | b8 | H |
| 2-2-512 | N-Biph | N-Biph | t-bu | t-bu | b8 | H |
| 2-2-513 | N-Biph | N-Biph | methyl | methyl | b8 | H |
| 2-2-514 | N-Biph | N-Biph | b1 | b1 | b8 | H |
| 2-2-515 | N-Biph | N-Biph | b2 | b2 | b8 | H |
| 2-2-516 | N-Biph | N-Biph | b3 | b3 | b8 | H |
| 2-2-517 | N-Biph | N-Biph | H | H | b9 | H |
| 2-2-518 | N-Biph | N-Biph | t-bu | t-bu | b9 | H |
| 2-2-519 | N-Biph | N-Biph | methyl | methyl | b9 | H |
| 2-2-520 | N-Biph | N-Biph | b1 | b1 | b9 | H |
| 2-2-521 | N-Biph | N-Biph | b2 | b2 | b9 | H |
| 2-2-522 | N-Biph | N-Biph | b3 | b3 | b9 | H |
| 2-2-523 | N-Biph | N-Biph | H | H | b10 | H |
| 2-2-524 | N-Biph | N-Biph | t-bu | t-bu | b10 | H |
| 2-2-525 | N-Biph | N-Biph | methyl | methyl | b10 | H |
| 2-2-526 | N-Biph | N-Biph | b1 | b1 | b10 | H |
| 2-2-527 | N-Biph | N-Biph | b2 | b2 | b10 | H |
| 2-2-528 | N-Biph | N-Biph | b3 | b3 | b10 | H |
| 2-2-529 | N-Biph | N-Biph | H | H | a1 | a1 |
| 2-2-530 | N-Biph | N-Biph | t-bu | t-bu | a1 | a1 |
| 2-2-531 | N-Biph | N-Biph | methyl | methyl | a1 | a1 |
| 2-2-532 | N-Biph | N-Biph | b1 | b1 | a1 | a1 |
| 2-2-533 | N-Biph | N-Biph | b2 | b2 | a1 | a1 |
| 2-2-534 | N-Biph | N-Biph | b3 | b3 | a1 | a1 |
| 2-2-535 | N-Biph | N-Biph | H | H | a2 | a2 |
| 2-2-536 | N-Biph | N-Biph | t-bu | t-bu | a2 | a2 |
| 2-2-537 | N-Biph | N-Biph | methyl | methyl | a2 | a2 |
| 2-2-538 | N-Biph | N-Biph | b1 | b1 | a2 | a2 |
| 2-2-539 | N-Biph | N-Biph | b2 | b2 | a2 | a2 |
| 2-2-540 | N-Biph | N-Biph | b3 | b3 | a2 | a2 |
| 2-2-541 | N-Biph | N-Biph | H | H | a3 | a3 |
| 2-2-542 | N-Biph | N-Biph | t-bu | t-bu | a3 | a3 |
| 2-2-543 | N-Biph | N-Biph | methyl | methyl | a3 | a3 |
| 2-2-544 | N-Biph | N-Biph | b1 | b1 | a3 | a3 |
| 2-2-545 | N-Biph | N-Biph | b2 | b2 | a3 | a3 |
| 2-2-546 | N-Biph | N-Biph | b3 | b3 | a3 | a3 |
| 2-2-547 | N-Biph | N-Biph | H | H | b1 | b1 |
| 2-2-548 | N-Biph | N-Biph | t-bu | t-bu | b1 | b1 |
| 2-2-549 | N-Biph | N-Biph | methyl | methyl | b1 | b1 |
| 2-2-550 | N-Biph | N-Biph | b1 | b1 | b1 | b1 |
| 2-2-551 | N-Biph | N-Biph | b2 | b2 | b1 | b1 |
| 2-2-552 | N-Biph | N-Biph | b3 | b3 | b1 | b1 |
| 2-2-553 | N-Biph | N-Biph | H | H | b2 | b2 |
| 2-2-554 | N-Biph | N-Biph | t-bu | t-bu | b2 | b2 |
| 2-2-555 | N-Biph | N-Biph | methyl | methyl | b2 | b2 |
| 2-2-556 | N-Biph | N-Biph | b1 | b1 | b2 | b2 |
| 2-2-557 | N-Biph | N-Biph | b2 | b2 | b2 | b2 |
| 2-2-558 | N-Biph | N-Biph | b3 | b3 | b2 | b2 |
| 2-2-559 | N-Biph | N-Biph | H | H | b3 | b3 |
| 2-2-560 | N-Biph | N-Biph | t-bu | t-bu | b3 | b3 |
| 2-2-561 | N-Biph | N-Biph | methyl | methyl | b3 | b3 |
| 2-2-562 | N-Biph | N-Biph | b1 | b1 | b3 | b3 |
| 2-2-563 | N-Biph | N-Biph | b2 | b2 | b3 | b3 |
| 2-2-564 | N-Biph | N-Biph | b3 | b3 | b3 | b3 |
| 2-2-565 | N-Biph | N-Biph | H | H | b5 | b5 |
| 2-2-566 | N-Biph | N-Biph | t-bu | t-bu | b5 | b5 |
| 2-2-567 | N-Biph | N-Biph | methyl | methyl | b5 | b5 |
| 2-2-568 | N-Biph | N-Biph | b1 | b1 | b5 | b5 |
| 2-2-569 | N-Biph | N-Biph | b2 | b2 | b5 | b5 |
| 2-2-570 | N-Biph | N-Biph | b3 | b3 | b5 | b5 |
| 2-2-571 | N-Biph | N-Biph | H | H | b6 | b6 |
| 2-2-572 | N-Biph | N-Biph | t-bu | t-bu | b6 | b6 |
| 2-2-573 | N-Biph | N-Biph | methyl | methyl | b6 | b6 |
| 2-2-574 | N-Biph | N-Biph | b1 | b1 | b6 | b6 |
| 2-2-575 | N-Biph | N-Biph | b2 | b2 | b6 | b6 |
| 2-2-576 | N-Biph | N-Biph | b3 | b3 | b6 | b6 |
| 2-2-577 | N-Biph | N-Biph | H | H | b7 | b7 |
| 2-2-578 | N-Biph | N-Biph | t-bu | t-bu | b7 | b7 |
| 2-2-579 | N-Biph | N-Biph | methyl | methyl | b7 | b7 |
| 2-2-580 | N-Biph | N-Biph | b1 | b1 | b7 | b7 |
| 2-2-581 | N-Biph | N-Biph | b2 | b2 | b7 | b7 |
| 2-2-582 | N-Biph | N-Biph | b3 | b3 | b7 | b7 |
| 2-2-583 | O | N—Ph | H | H | H | H |
| 2-2-584 | O | N—Ph | t-bu | t-bu | H | H |
| 2-2-585 | O | N—Ph | methyl | methyl | H | H |
| 2-2-586 | O | N—Ph | t-bu | t-bu | t-bu | t-bu |
| 2-2-587 | O | N—Ph | methyl | methyl | methyl | methyl |
| 2-2-588 | O | N—Ph | b1 | b1 | H | H |
| 2-2-589 | O | N—Ph | b2 | b2 | H | H |
| 2-2-590 | O | N—Ph | b3 | b3 | H | H |
| 2-2-591 | O | N—Ph | H | H | H | a1 |
| 2-2-592 | O | N—Ph | t-bu | t-bu | H | a1 |
| 2-2-593 | O | N—Ph | methyl | methyl | H | a1 |
| 2-2-594 | O | N—Ph | b1 | b1 | H | a1 |
| 2-2-595 | O | N—Ph | b2 | b2 | H | a1 |
| 2-2-596 | O | N—Ph | b3 | b3 | H | a1 |
| 2-2-597 | O | N—Ph | H | H | H | a2 |
| 2-2-598 | O | N—Ph | t-bu | t-bu | H | a2 |
| 2-2-599 | O | N—Ph | methyl | methyl | H | a2 |
| 2-2-600 | O | N—Ph | b1 | b1 | H | a2 |
| 2-2-601 | O | N—Ph | b2 | b2 | H | a2 |
| 2-2-602 | O | N—Ph | b3 | b3 | H | a2 |
| 2-2-603 | O | N—Ph | H | H | H | a3 |
| 2-2-604 | O | N—Ph | t-bu | t-bu | H | a3 |
| 2-2-605 | O | N—Ph | methyl | methyl | H | a3 |
| 2-2-606 | O | N—Ph | b1 | b1 | H | a3 |
| 2-2-607 | O | N—Ph | b2 | b2 | H | a3 |
| 2-2-608 | O | N—Ph | b3 | b3 | H | a3 |
| 2-2-609 | O | N—Ph | H | H | H | a4 |
| 2-2-610 | O | N—Ph | t-bu | t-bu | H | a4 |
| 2-2-611 | O | N—Ph | methyl | methyl | H | a4 |
| 2-2-612 | O | N—Ph | b1 | b1 | H | a4 |
| 2-2-613 | O | N—Ph | b2 | b2 | H | a4 |
| 2-2-614 | O | N—Ph | b3 | b3 | H | a4 |
| 2-2-615 | O | N—Ph | H | H | H | a5 |
| 2-2-616 | O | N—Ph | t-bu | t-bu | H | a5 |
| 2-2-617 | O | N—Ph | methyl | methyl | H | a5 |
| 2-2-618 | O | N—Ph | b1 | b1 | H | a5 |
| 2-2-619 | O | N—Ph | b2 | b2 | H | a5 |
| 2-2-620 | O | N—Ph | b3 | b3 | H | a5 |
| 2-2-621 | O | N—Ph | H | H | H | a6 |
| 2-2-622 | O | N—Ph | t-bu | t-bu | H | a6 |
| 2-2-623 | O | N—Ph | methyl | methyl | H | a6 |
| 2-2-624 | O | N—Ph | b1 | b1 | H | a6 |
| 2-2-625 | O | N—Ph | b2 | b2 | H | a6 |
| 2-2-626 | O | N—Ph | b3 | b3 | H | a6 |
| 2-2-627 | O | N—Ph | H | H | a1 | H |
| 2-2-628 | O | N—Ph | t-bu | t-bu | a1 | H |
| 2-2-629 | O | N—Ph | methyl | methyl | a1 | H |
| 2-2-630 | O | N—Ph | b1 | b1 | a1 | H |
| 2-2-631 | O | N—Ph | b2 | b2 | a1 | H |
| 2-2-632 | O | N—Ph | b3 | b3 | a1 | H |
| 2-2-633 | O | N—Ph | H | H | a2 | H |
| 2-2-634 | O | N—Ph | t-bu | t-bu | a2 | H |
| 2-2-635 | O | N—Ph | methyl | methyl | a2 | H |
| 2-2-636 | O | N—Ph | b1 | b1 | a2 | H |
| 2-2-637 | O | N—Ph | b2 | b2 | a2 | H |
| 2-2-638 | O | N—Ph | b3 | b3 | a2 | H |
| 2-2-639 | O | N—Ph | H | H | a3 | H |
| 2-2-640 | O | N—Ph | t-bu | t-bu | a3 | H |
| 2-2-641 | O | N—Ph | methyl | methyl | a3 | H |
| 2-2-642 | O | N—Ph | b1 | b1 | a3 | H |
| 2-2-643 | O | N—Ph | b2 | b2 | a3 | H |
| 2-2-644 | O | N—Ph | b3 | b3 | a3 | H |
| 2-2-645 | O | N—Ph | H | H | a4 | H |
| 2-2-646 | O | N—Ph | t-bu | t-bu | a4 | H |
| 2-2-647 | O | N—Ph | methyl | methyl | a4 | H |
| 2-2-648 | O | N—Ph | b1 | b1 | a4 | H |
| 2-2-649 | O | N—Ph | b2 | b2 | a4 | H |
| 2-2-650 | O | N—Ph | b3 | b3 | a4 | H |
| 2-2-651 | O | N—Ph | H | H | a5 | H |
| 2-2-652 | O | N—Ph | t-bu | t-bu | a5 | H |
| 2-2-653 | O | N—Ph | methyl | methyl | a5 | H |
| 2-2-654 | O | N—Ph | b1 | b1 | a5 | H |
| 2-2-655 | O | N—Ph | b2 | b2 | a5 | H |
| 2-2-656 | O | N—Ph | b3 | b3 | a5 | H |
| 2-2-657 | O | N—Ph | H | H | a6 | H |
| 2-2-658 | O | N—Ph | t-bu | t-bu | a6 | H |
| 2-2-659 | O | N—Ph | methyl | methyl | a6 | H |
| 2-2-660 | O | N—Ph | b1 | b1 | a6 | H |
| 2-2-661 | O | N—Ph | b2 | b2 | a6 | H |
| 2-2-662 | O | N—Ph | b3 | b3 | a6 | H |
| 2-2-663 | O | N—Ph | H | H | b1 | H |
| 2-2-664 | O | N—Ph | t-bu | t-bu | b1 | H |
| 2-2-665 | O | N—Ph | methyl | methyl | b1 | H |
| 2-2-666 | O | N—Ph | b1 | b1 | b1 | H |
| 2-2-667 | O | N—Ph | b2 | b2 | b1 | H |
| 2-2-668 | O | N—Ph | b3 | b3 | b1 | H |
| 2-2-669 | O | N—Ph | H | H | b2 | H |
| 2-2-670 | O | N—Ph | t-bu | t-bu | b2 | H |
| 2-2-671 | O | N—Ph | methyl | methyl | b2 | H |
| 2-2-672 | O | N—Ph | b1 | b1 | b2 | H |
| 2-2-673 | O | N—Ph | b2 | b2 | b2 | H |
| 2-2-674 | O | N—Ph | b3 | b3 | b2 | H |
| 2-2-675 | O | N—Ph | H | H | b3 | H |
| 2-2-676 | O | N—Ph | t-bu | t-bu | b3 | H |
| 2-2-677 | O | N—Ph | methyl | methyl | b3 | H |
| 2-2-678 | O | N—Ph | b1 | b1 | b3 | H |
| 2-2-679 | O | N—Ph | b2 | b2 | b3 | H |
| 2-2-680 | O | N—Ph | b3 | b3 | b3 | H |
| 2-2-681 | O | N—Ph | H | H | b4 | H |
| 2-2-682 | O | N—Ph | t-bu | t-bu | b4 | H |
| 2-2-683 | O | N—Ph | methyl | methyl | b4 | H |
| 2-2-684 | O | N—Ph | b1 | b1 | b4 | H |
| 2-2-685 | O | N—Ph | b2 | b2 | b4 | H |
| 2-2-686 | O | N—Ph | b3 | b3 | b4 | H |
| 2-2-687 | O | N—Ph | H | H | b5 | H |
| 2-2-688 | O | N—Ph | t-bu | t-bu | b5 | H |
| 2-2-689 | O | N—Ph | methyl | methyl | b5 | H |
| 2-2-690 | O | N—Ph | b1 | b1 | b5 | H |
| 2-2-691 | O | N—Ph | b2 | b2 | b5 | H |
| 2-2-692 | O | N—Ph | b3 | b3 | b5 | H |
| 2-2-693 | O | N—Ph | H | H | b6 | H |
| 2-2-694 | O | N—Ph | t-bu | t-bu | b6 | H |
| 2-2-695 | O | N—Ph | methyl | methyl | b6 | H |
| 2-2-696 | O | N—Ph | b1 | b1 | b6 | H |
| 2-2-697 | O | N—Ph | b2 | b2 | b6 | H |
| 2-2-698 | O | N—Ph | b3 | b3 | b6 | H |
| 2-2-699 | O | N—Ph | H | H | b7 | H |
| 2-2-700 | O | N—Ph | t-bu | t-bu | b7 | H |
| 2-2-701 | O | N—Ph | methyl | methyl | b7 | H |
| 2-2-702 | O | N—Ph | b1 | b1 | b7 | H |
| 2-2-703 | O | N—Ph | b2 | b2 | b7 | H |
| 2-2-704 | O | N—Ph | b3 | b3 | b7 | H |
| 2-2-705 | O | N—Ph | H | H | b8 | H |
| 2-2-706 | O | N—Ph | t-bu | t-bu | b8 | H |
| 2-2-707 | O | N—Ph | methyl | methyl | b8 | H |
| 2-2-708 | O | N—Ph | b1 | b1 | b8 | H |
| 2-2-709 | O | N—Ph | b2 | b2 | b8 | H |
| 2-2-710 | O | N—Ph | b3 | b3 | b8 | H |
| 2-2-711 | O | N—Ph | H | H | b9 | H |
| 2-2-712 | O | N—Ph | t-bu | t-bu | b9 | H |
| 2-2-713 | O | N—Ph | methyl | methyl | b9 | H |
| 2-2-714 | O | N—Ph | b1 | b1 | b9 | H |
| 2-2-715 | O | N—Ph | b2 | b2 | b9 | H |
| 2-2-716 | O | N—Ph | b3 | b3 | b9 | H |
| 2-2-717 | O | N—Ph | H | H | b10 | H |
| 2-2-718 | O | N—Ph | t-bu | t-bu | b10 | H |
| 2-2-719 | O | N—Ph | methyl | methyl | b10 | H |
| 2-2-720 | O | N—Ph | b1 | b1 | b10 | H |
| 2-2-721 | O | N—Ph | b2 | b2 | b10 | H |
| 2-2-722 | O | N—Ph | b3 | b3 | b10 | H |
| 2-2-723 | O | N—Ph | H | H | a1 | a1 |
| 2-2-724 | O | N—Ph | t-bu | t-bu | a1 | a1 |
| 2-2-725 | O | N—Ph | methyl | methyl | a1 | a1 |
| 2-2-726 | O | N—Ph | b1 | b1 | a1 | a1 |
| 2-2-727 | O | N—Ph | b2 | b2 | a1 | a1 |
| 2-2-728 | O | N—Ph | b3 | b3 | a1 | a1 |
| 2-2-729 | O | N—Ph | H | H | a2 | a2 |
| 2-2-730 | O | N—Ph | t-bu | t-bu | a2 | a2 |
| 2-2-731 | O | N—Ph | methyl | methyl | a2 | a2 |
| 2-2-732 | O | N—Ph | b1 | b1 | a2 | a2 |
| 2-2-733 | O | N—Ph | b2 | b2 | a2 | a2 |
| 2-2-734 | O | N—Ph | b3 | b3 | a2 | a2 |
| 2-2-735 | O | N—Ph | H | H | a3 | a3 |
| 2-2-736 | O | N—Ph | t-bu | t-bu | a3 | a3 |
| 2-2-737 | O | N—Ph | methyl | methyl | a3 | a3 |
| 2-2-738 | O | N—Ph | b1 | b1 | a3 | a3 |
| 2-2-739 | O | N—Ph | b2 | b2 | a3 | a3 |
| 2-2-740 | O | N—Ph | b3 | b3 | a3 | a3 |
| 2-2-741 | O | N—Ph | H | H | b1 | b1 |
| 2-2-742 | O | N—Ph | t-bu | t-bu | b1 | b1 |
| 2-2-743 | O | N—Ph | methyl | methyl | b1 | b1 |
| 2-2-744 | O | N—Ph | b1 | b1 | b1 | b1 |
| 2-2-745 | O | N—Ph | b2 | b2 | b1 | b1 |
| 2-2-746 | O | N—Ph | b3 | b3 | b1 | b1 |
| 2-2-747 | O | N—Ph | H | H | b2 | b2 |
| 2-2-748 | O | N—Ph | t-bu | t-bu | b2 | b2 |
| 2-2-749 | O | N—Ph | methyl | methyl | b2 | b2 |
| 2-2-750 | O | N—Ph | b1 | b1 | b2 | b2 |
| 2-2-751 | O | N—Ph | b2 | b2 | b2 | b2 |
| 2-2-752 | O | N—Ph | b3 | b3 | b2 | b2 |
| 2-2-753 | O | N—Ph | H | H | b3 | b3 |
| 2-2-754 | O | N—Ph | t-bu | t-bu | b3 | b3 |
| 2-2-755 | O | N—Ph | methyl | methyl | b3 | b3 |
| 2-2-756 | O | N—Ph | b1 | b1 | b3 | b3 |
| 2-2-757 | O | N—Ph | b2 | b2 | b3 | b3 |
| 2-2-758 | O | N—Ph | b3 | b3 | b3 | b3 |
| 2-2-759 | O | N—Ph | H | H | b5 | b5 |
| 2-2-760 | O | N—Ph | t-bu | t-bu | b5 | b5 |
| 2-2-761 | O | N—Ph | methyl | methyl | b5 | b5 |
| 2-2-762 | O | N—Ph | b1 | b1 | b5 | b5 |
| 2-2-763 | O | N—Ph | b2 | b2 | b5 | b5 |
| 2-2-764 | O | N—Ph | b3 | b3 | b5 | b5 |
| 2-2-765 | O | N—Ph | H | H | b6 | b6 |
| 2-2-766 | O | N—Ph | t-bu | t-bu | b6 | b6 |
| 2-2-767 | O | N—Ph | methyl | methyl | b6 | b6 |
| 2-2-768 | O | N—Ph | b1 | b1 | b6 | b6 |
| 2-2-769 | O | N—Ph | b2 | b2 | b6 | b6 |
| 2-2-770 | O | N—Ph | b3 | b3 | b6 | b6 |
| 2-2-771 | O | N—Ph | H | H | b7 | b7 |
| 2-2-772 | O | N—Ph | t-bu | t-bu | b7 | b7 |
| 2-2-773 | O | N—Ph | methyl | methyl | b7 | b7 |
| 2-2-774 | O | N—Ph | b1 | b1 | b7 | b7 |
| 2-2-775 | O | N—Ph | b2 | b2 | b7 | b7 |
| 2-2-776 | O | N—Ph | b3 | b3 | b7 | b7 |
| 2-2-777 | S | N—Ph | H | H | H | H |
| 2-2-778 | S | N—Ph | t-bu | t-bu | H | H |
| 2-2-779 | S | N—Ph | methyl | methyl | H | H |
| 2-2-780 | S | N—Ph | t-bu | t-bu | t-bu | t-bu |
| 2-2-781 | S | N—Ph | methyl | methyl | methyl | methyl |
| 2-2-782 | S | N—Ph | b1 | b1 | H | H |
| 2-2-783 | S | N—Ph | b2 | b2 | H | H |
| 2-2-784 | S | N—Ph | b3 | b3 | H | H |
| 2-2-785 | S | N—Ph | H | H | H | a1 |
| 2-2-786 | S | N—Ph | t-bu | t-bu | H | a1 |
| 2-2-787 | S | N—Ph | methyl | methyl | H | a1 |
| 2-2-788 | S | N—Ph | b1 | b1 | H | a1 |
| 2-2-789 | S | N—Ph | b2 | b2 | H | a1 |
| 2-2-790 | S | N—Ph | b3 | b3 | H | a1 |
| 2-2-791 | S | N—Ph | H | H | H | a2 |
| 2-2-792 | S | N—Ph | t-bu | t-bu | H | a2 |
| 2-2-793 | S | N—Ph | methyl | methyl | H | a2 |
| 2-2-794 | S | N—Ph | b1 | b1 | H | a2 |
| 2-2-795 | S | N—Ph | b2 | b2 | H | a2 |
| 2-2-796 | S | N—Ph | b3 | b3 | H | a2 |
| 2-2-797 | S | N—Ph | H | H | H | a3 |
| 2-2-798 | S | N—Ph | t-bu | t-bu | H | a3 |
| 2-2-799 | S | N—Ph | methyl | methyl | H | a3 |
| 2-2-800 | S | N—Ph | b1 | b1 | H | a3 |
| 2-2-801 | S | N—Ph | b2 | b2 | H | a3 |
| 2-2-802 | S | N—Ph | b3 | b3 | H | a3 |
| 2-2-803 | S | N—Ph | H | H | H | a4 |
| 2-2-804 | S | N—Ph | t-bu | t-bu | H | a4 |
| 2-2-805 | S | N—Ph | methyl | methyl | H | a4 |
| 2-2-806 | S | N—Ph | b1 | b1 | H | a4 |
| 2-2-807 | S | N—Ph | b2 | b2 | H | a4 |
| 2-2-808 | S | N—Ph | b3 | b3 | H | a4 |
| 2-2-809 | S | N—Ph | H | H | H | a5 |
| 2-2-810 | S | N—Ph | t-bu | t-bu | H | a5 |
| 2-2-811 | S | N—Ph | methyl | methyl | H | a5 |
| 2-2-812 | S | N—Ph | b1 | b1 | H | a5 |
| 2-2-813 | S | N—Ph | b2 | b2 | H | a5 |
| 2-2-814 | S | N—Ph | b3 | b3 | H | a5 |
| 2-2-815 | S | N—Ph | H | H | H | a6 |
| 2-2-816 | S | N—Ph | t-bu | t-bu | H | a6 |
| 2-2-817 | S | N—Ph | methyl | methyl | H | a6 |
| 2-2-818 | S | N—Ph | b1 | b1 | H | a6 |
| 2-2-819 | S | N—Ph | b2 | b2 | H | a6 |
| 2-2-820 | S | N—Ph | b3 | b3 | H | a6 |
| 2-2-821 | S | N—Ph | H | H | a1 | H |
| 2-2-822 | S | N—Ph | t-bu | t-bu | a1 | H |
| 2-2-823 | S | N—Ph | methyl | methyl | a1 | H |
| 2-2-824 | S | N—Ph | b1 | b1 | a1 | H |
| 2-2-825 | S | N—Ph | b2 | b2 | a1 | H |
| 2-2-826 | S | N—Ph | b3 | b3 | a1 | H |
| 2-2-827 | S | N—Ph | H | H | a2 | H |
| 2-2-828 | S | N—Ph | t-bu | t-bu | a2 | H |
| 2-2-829 | S | N—Ph | methyl | methyl | a2 | H |
| 2-2-830 | S | N—Ph | b1 | b1 | a2 | H |
| 2-2-831 | S | N—Ph | b2 | b2 | a2 | H |
| 2-2-832 | S | N—Ph | b3 | b3 | a2 | H |
| 2-2-833 | S | N—Ph | H | H | a3 | H |
| 2-2-834 | S | N—Ph | t-bu | t-bu | a3 | H |
| 2-2-835 | S | N—Ph | methyl | methyl | a3 | H |
| 2-2-836 | S | N—Ph | b1 | b1 | a3 | H |
| 2-2-837 | S | N—Ph | b2 | b2 | a3 | H |
| 2-2-838 | S | N—Ph | b3 | b3 | a3 | H |
| 2-2-839 | S | N—Ph | H | H | a4 | H |
| 2-2-840 | S | N—Ph | t-bu | t-bu | a4 | H |
| 2-2-841 | S | N—Ph | methyl | methyl | a4 | H |
| 2-2-842 | S | N—Ph | b1 | b1 | a4 | H |
| 2-2-843 | S | N—Ph | b2 | b2 | a4 | H |
| 2-2-844 | S | N—Ph | b3 | b3 | a4 | H |
| 2-2-845 | S | N—Ph | H | H | a5 | H |
| 2-2-846 | S | N—Ph | t-bu | t-bu | a5 | H |
| 2-2-847 | S | N—Ph | methyl | methyl | a5 | H |
| 2-2-848 | S | N—Ph | b1 | b1 | a5 | H |
| 2-2-849 | S | N—Ph | b2 | b2 | a5 | H |
| 2-2-850 | S | N—Ph | b3 | b3 | a5 | H |
| 2-2-851 | S | N—Ph | H | H | a6 | H |
| 2-2-852 | S | N—Ph | t-bu | t-bu | a6 | H |
| 2-2-853 | S | N—Ph | methyl | methyl | a6 | H |
| 2-2-854 | S | N—Ph | b1 | b1 | a6 | H |
| 2-2-855 | S | N—Ph | b2 | b2 | a6 | H |
| 2-2-856 | S | N—Ph | b3 | b3 | a6 | H |
| 2-2-857 | S | N—Ph | H | H | b1 | H |
| 2-2-858 | S | N—Ph | t-bu | t-bu | b1 | H |
| 2-2-859 | S | N—Ph | methyl | methyl | b1 | H |
| 2-2-860 | S | N—Ph | b1 | b1 | b1 | H |
| 2-2-861 | S | N—Ph | b2 | b2 | b1 | H |
| 2-2-862 | S | N—Ph | b3 | b3 | b1 | H |
| 2-2-863 | S | N—Ph | H | H | b2 | H |
| 2-2-864 | S | N—Ph | t-bu | t-bu | b2 | H |
| 2-2-865 | S | N—Ph | methyl | methyl | b2 | H |
| 2-2-866 | S | N—Ph | b1 | b1 | b2 | H |
| 2-2-867 | S | N—Ph | b2 | b2 | b2 | H |
| 2-2-868 | S | N—Ph | b3 | b3 | b2 | H |
| 2-2-869 | S | N—Ph | H | H | b3 | H |
| 2-2-870 | S | N—Ph | t-bu | t-bu | b3 | H |
| 2-2-871 | S | N—Ph | methyl | methyl | b3 | H |
| 2-2-872 | S | N—Ph | b1 | b1 | b3 | H |
| 2-2-873 | S | N—Ph | b2 | b2 | b3 | H |
| 2-2-874 | S | N—Ph | b3 | b3 | b3 | H |
| 2-2-875 | S | N—Ph | H | H | b4 | H |
| 2-2-876 | S | N—Ph | t-bu | t-bu | b4 | H |
| 2-2-877 | S | N—Ph | methyl | methyl | b4 | H |
| 2-2-878 | S | N—Ph | b1 | b1 | b4 | H |
| 2-2-879 | S | N—Ph | b2 | b2 | b4 | H |
| 2-2-880 | S | N—Ph | b3 | b3 | b4 | H |
| 2-2-881 | S | N—Ph | H | H | b5 | H |
| 2-2-882 | S | N—Ph | t-bu | t-bu | b5 | H |
| 2-2-883 | S | N—Ph | methyl | methyl | b5 | H |
| 2-2-884 | S | N—Ph | b1 | b1 | b5 | H |
| 2-2-885 | S | N—Ph | b2 | b2 | b5 | H |
| 2-2-886 | S | N—Ph | b3 | b3 | b5 | H |
| 2-2-887 | S | N—Ph | H | H | b6 | H |
| 2-2-888 | S | N—Ph | t-bu | t-bu | b6 | H |
| 2-2-889 | S | N—Ph | methyl | methyl | b6 | H |
| 2-2-890 | S | N—Ph | b1 | b1 | b6 | H |
| 2-2-891 | S | N—Ph | b2 | b2 | b6 | H |
| 2-2-892 | S | N—Ph | b3 | b3 | b6 | H |
| 2-2-893 | S | N—Ph | H | H | b7 | H |
| 2-2-894 | S | N—Ph | t-bu | t-bu | b7 | H |
| 2-2-895 | S | N—Ph | methyl | methyl | b7 | H |
| 2-2-896 | S | N—Ph | b1 | b1 | b7 | H |
| 2-2-897 | S | N—Ph | b2 | b2 | b7 | H |
| 2-2-898 | S | N—Ph | b3 | b3 | b7 | H |
| 2-2-899 | S | N—Ph | H | H | b8 | H |
| 2-2-900 | S | N—Ph | t-bu | t-bu | b8 | H |
| 2-2-901 | S | N—Ph | methyl | methyl | b8 | H |
| 2-2-902 | S | N—Ph | b1 | b1 | b8 | H |
| 2-2-903 | S | N—Ph | b2 | b2 | b8 | H |
| 2-2-904 | S | N—Ph | b3 | b3 | b8 | H |
| 2-2-905 | S | N—Ph | H | H | b9 | H |
| 2-2-906 | S | N—Ph | t-bu | t-bu | b9 | H |
| 2-2-907 | S | N—Ph | methyl | methyl | b9 | H |
| 2-2-908 | S | N—Ph | b1 | b1 | b9 | H |
| 2-2-909 | S | N—Ph | b2 | b2 | b9 | H |
| 2-2-910 | S | N—Ph | b3 | b3 | b9 | H |
| 2-2-911 | S | N—Ph | H | H | b10 | H |
| 2-2-912 | S | N—Ph | t-bu | t-bu | b10 | H |
| 2-2-913 | S | N—Ph | methyl | methyl | b10 | H |
| 2-2-914 | S | N—Ph | b1 | b1 | b10 | H |
| 2-2-915 | S | N—Ph | b2 | b2 | b10 | H |
| 2-2-916 | S | N—Ph | b3 | b3 | b10 | H |
| 2-2-917 | S | N—Ph | H | H | a1 | a1 |
| 2-2-918 | S | N—Ph | t-bu | t-bu | a1 | a1 |
| 2-2-919 | S | N—Ph | methyl | methyl | a1 | a1 |
| 2-2-920 | S | N—Ph | b1 | b1 | a1 | a1 |
| 2-2-921 | S | N—Ph | b2 | b2 | a1 | a1 |
| 2-2-922 | S | N—Ph | b3 | b3 | a1 | a1 |
| 2-2-923 | S | N—Ph | H | H | a2 | a2 |
| 2-2-924 | S | N—Ph | t-bu | t-bu | a2 | a2 |
| 2-2-925 | S | N—Ph | methyl | methyl | a2 | a2 |
| 2-2-926 | S | N—Ph | b1 | b1 | a2 | a2 |
| 2-2-927 | S | N—Ph | b2 | b2 | a2 | a2 |
| 2-2-928 | S | N—Ph | b3 | b3 | a2 | a2 |
| 2-2-929 | S | N—Ph | H | H | a3 | a3 |
| 2-2-930 | S | N—Ph | t-bu | t-bu | a3 | a3 |
| 2-2-931 | S | N—Ph | methyl | methyl | a3 | a3 |
| 2-2-932 | S | N—Ph | b1 | b1 | a3 | a3 |
| 2-2-933 | S | N—Ph | b2 | b2 | a3 | a3 |
| 2-2-934 | S | N—Ph | b3 | b3 | a3 | a3 |
| 2-2-935 | S | N—Ph | H | H | b1 | b1 |
| 2-2-936 | S | N—Ph | t-bu | t-bu | b1 | b1 |
| 2-2-937 | S | N—Ph | methyl | methyl | b1 | b1 |
| 2-2-938 | S | N—Ph | b1 | b1 | b1 | b1 |
| 2-2-939 | S | N—Ph | b2 | b2 | b1 | b1 |
| 2-2-940 | S | N—Ph | b3 | b3 | b1 | b1 |
| 2-2-941 | S | N—Ph | H | H | b2 | b2 |
| 2-2-942 | S | N—Ph | t-bu | t-bu | b2 | b2 |
| 2-2-943 | S | N—Ph | methyl | methyl | b2 | b2 |
| 2-2-944 | S | N—Ph | b1 | b1 | b2 | b2 |
| 2-2-945 | S | N—Ph | b2 | b2 | b2 | b2 |
| 2-2-946 | S | N—Ph | b3 | b3 | b2 | b2 |
| 2-2-947 | S | N—Ph | H | H | b3 | b3 |
| 2-2-948 | S | N—Ph | t-bu | t-bu | b3 | b3 |
| 2-2-949 | S | N—Ph | methyl | methyl | b3 | b3 |
| 2-2-950 | S | N—Ph | b1 | b1 | b3 | b3 |
| 2-2-951 | S | N—Ph | b2 | b2 | b3 | b3 |
| 2-2-952 | S | N—Ph | b3 | b3 | b3 | b3 |
| 2-2-953 | S | N—Ph | H | H | b5 | b5 |
| 2-2-954 | S | N—Ph | t-bu | t-bu | b5 | b5 |
| 2-2-955 | S | N—Ph | methyl | methyl | b5 | b5 |
| 2-2-956 | S | N—Ph | b1 | b1 | b5 | b5 |
| 2-2-957 | S | N—Ph | b2 | b2 | b5 | b5 |
| 2-2-958 | S | N—Ph | b3 | b3 | b5 | b5 |
| 2-2-959 | S | N—Ph | H | H | b6 | b6 |
| 2-2-960 | S | N—Ph | t-bu | t-bu | b6 | b6 |
| 2-2-961 | S | N—Ph | methyl | methyl | b6 | b6 |
| 2-2-962 | S | N—Ph | b1 | b1 | b6 | b6 |
| 2-2-963 | S | N—Ph | b2 | b2 | b6 | b6 |
| 2-2-964 | S | N—Ph | b3 | b3 | b6 | b6 |
| 2-2-965 | S | N—Ph | H | H | b7 | b7 |
| 2-2-966 | S | N—Ph | t-bu | t-bu | b7 | b7 |
| 2-2-967 | S | N—Ph | methyl | methyl | b7 | b7 |
| 2-2-968 | S | N—Ph | b1 | b1 | b7 | b7 |
| 2-2-969 | S | N—Ph | b2 | b2 | b7 | b7 |
| 2-2-970 | S | N—Ph | b3 | b3 | b7 | b7 |

### Group 2-3

The compounds are represented by the formula 2-3 below, wherein CY₁ and CY₃ are each a benzene group, X₁ is B, and Y1, Y2, R2b, R2d, (R1)a1, (R3)a3 are each shown in the table 3 below,

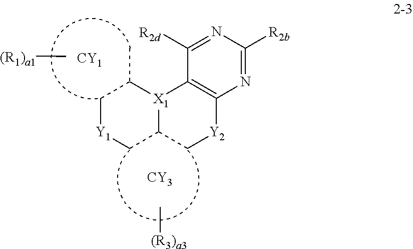

TABLE 3

| Compound No. | Y1 | Y2 | R2b | R2d | (R1)a1 | (R3)a3 |
|---|---|---|---|---|---|---|
| 2-3-001 | N—Ph | N—Ph | H | H | H | H |
| 2-3-002 | N—Ph | N—Ph | t-bu | H | H | H |
| 2-3-003 | N—Ph | N—Ph | methyl | H | H | H |
| 2-3-004 | N—Ph | N—Ph | t-bu | t-bu | t-bu | t-bu |
| 2-3-005 | N—Ph | N—Ph | methyl | methyl | methyl | methyl |
| 2-3-006 | N—Ph | N—Ph | b1 | H | H | H |
| 2-3-007 | N—Ph | N—Ph | b2 | H | H | H |
| 2-3-008 | N—Ph | N—Ph | b3 | H | H | H |
| 2-3-009 | N—Ph | N—Ph | H | H | H | a1 |
| 2-3-010 | N—Ph | N—Ph | t-bu | H | H | a1 |
| 2-3-011 | N—Ph | N—Ph | methyl | H | H | a1 |
| 2-3-012 | N—Ph | N—Ph | b1 | H | H | a1 |
| 2-3-013 | N—Ph | N—Ph | b2 | H | H | a1 |
| 2-3-014 | N—Ph | N—Ph | b3 | H | H | a1 |
| 2-3-015 | N—Ph | N—Ph | H | H | H | a2 |
| 2-3-016 | N—Ph | N—Ph | t-bu | H | H | a2 |
| 2-3-017 | N—Ph | N—Ph | methyl | H | H | a2 |
| 2-3-018 | N—Ph | N—Ph | b1 | H | H | a2 |
| 2-3-019 | N—Ph | N—Ph | b2 | H | H | a2 |
| 2-3-020 | N—Ph | N—Ph | b3 | H | H | a2 |
| 2-3-021 | N—Ph | N—Ph | H | H | H | a3 |
| 2-3-022 | N—Ph | N—Ph | t-bu | H | H | a3 |
| 2-3-023 | N—Ph | N—Ph | methyl | H | H | a3 |
| 2-3-024 | N—Ph | N—Ph | b1 | H | H | a3 |
| 2-3-025 | N—Ph | N—Ph | b2 | H | H | a3 |
| 2-3-026 | N—Ph | N—Ph | b3 | H | H | a3 |
| 2-3-027 | N—Ph | N—Ph | H | H | H | a4 |
| 2-3-028 | N—Ph | N—Ph | t-bu | H | H | a4 |
| 2-3-029 | N—Ph | N—Ph | methyl | H | H | a4 |
| 2-3-030 | N—Ph | N—Ph | b1 | H | H | a4 |
| 2-3-031 | N—Ph | N—Ph | b2 | H | H | a4 |
| 2-3-032 | N—Ph | N—Ph | b3 | H | H | a4 |
| 2-3-033 | N—Ph | N—Ph | H | H | H | a5 |
| 2-3-034 | N—Ph | N—Ph | t-bu | H | H | a5 |
| 2-3-035 | N—Ph | N—Ph | methyl | H | H | a5 |
| 2-3-036 | N—Ph | N—Ph | b1 | H | H | a5 |
| 2-3-037 | N—Ph | N—Ph | b2 | H | H | a5 |
| 2-3-038 | N—Ph | N—Ph | b3 | H | H | a5 |
| 2-3-039 | N—Ph | N—Ph | H | H | H | a6 |
| 2-3-040 | N—Ph | N—Ph | t-bu | H | H | a6 |
| 2-3-041 | N—Ph | N—Ph | methyl | H | H | a6 |
| 2-3-042 | N—Ph | N—Ph | b1 | H | H | a6 |
| 2-3-043 | N—Ph | N—Ph | b2 | H | H | a6 |
| 2-3-044 | N—Ph | N—Ph | b3 | H | H | a6 |
| 2-3-045 | N—Ph | N—Ph | H | H | a1 | H |
| 2-3-046 | N—Ph | N—Ph | t-bu | H | a1 | H |
| 2-3-047 | N—Ph | N—Ph | methyl | H | a1 | H |
| 2-3-048 | N—Ph | N—Ph | b1 | H | a1 | H |
| 2-3-049 | N—Ph | N—Ph | b2 | H | a1 | H |
| 2-3-050 | N—Ph | N—Ph | b3 | H | a1 | H |
| 2-3-051 | N—Ph | N—Ph | H | H | a2 | H |
| 2-3-052 | N—Ph | N—Ph | t-bu | H | a2 | H |
| 2-3-053 | N—Ph | N—Ph | methyl | H | a2 | H |
| 2-3-054 | N—Ph | N—Ph | b1 | H | a2 | H |
| 2-3-055 | N—Ph | N—Ph | b2 | H | a2 | H |
| 2-3-056 | N—Ph | N—Ph | b3 | H | a2 | H |
| 2-3-057 | N—Ph | N—Ph | H | H | a3 | H |
| 2-3-058 | N—Ph | N—Ph | t-bu | H | a3 | H |
| 2-3-059 | N—Ph | N—Ph | methyl | H | a3 | H |
| 2-3-060 | N—Ph | N—Ph | b1 | H | a3 | H |
| 2-3-061 | N—Ph | N—Ph | b2 | H | a3 | H |
| 2-3-062 | N—Ph | N—Ph | b3 | H | a3 | H |
| 2-3-063 | N—Ph | N—Ph | H | H | a4 | H |
| 2-3-064 | N—Ph | N—Ph | t-bu | H | a4 | H |
| 2-3-065 | N—Ph | N—Ph | methyl | H | a4 | H |
| 2-3-066 | N—Ph | N—Ph | b1 | H | a4 | H |
| 2-3-067 | N—Ph | N—Ph | b2 | H | a4 | H |
| 2-3-068 | N—Ph | N—Ph | b3 | H | a4 | H |
| 2-3-069 | N—Ph | N—Ph | H | H | a5 | H |
| 2-3-070 | N—Ph | N—Ph | t-bu | H | a5 | H |
| 2-3-071 | N—Ph | N—Ph | methyl | H | a5 | H |
| 2-3-072 | N—Ph | N—Ph | b1 | H | a5 | H |
| 2-3-073 | N—Ph | N—Ph | b2 | H | a5 | H |
| 2-3-074 | N—Ph | N—Ph | b3 | H | a5 | H |
| 2-3-075 | N—Ph | N—Ph | H | H | a6 | H |
| 2-3-076 | N—Ph | N—Ph | t-bu | H | a6 | H |
| 2-3-077 | N—Ph | N—Ph | methyl | H | a6 | H |
| 2-3-078 | N—Ph | N—Ph | b1 | H | a6 | H |
| 2-3-079 | N—Ph | N—Ph | b2 | H | a6 | H |
| 2-3-080 | N—Ph | N—Ph | b3 | H | a6 | H |
| 2-3-081 | N—Ph | N—Ph | H | H | b1 | H |
| 2-3-082 | N—Ph | N—Ph | t-bu | H | b1 | H |
| 2-3-083 | N—Ph | N—Ph | methyl | H | b1 | H |
| 2-3-084 | N—Ph | N—Ph | b1 | H | b1 | H |
| 2-3-085 | N—Ph | N—Ph | b2 | H | b1 | H |
| 2-3-086 | N—Ph | N—Ph | b3 | H | b1 | H |
| 2-3-087 | N—Ph | N—Ph | H | H | b2 | H |
| 2-3-088 | N—Ph | N—Ph | t-bu | H | b2 | H |
| 2-3-089 | N—Ph | N—Ph | methyl | H | b2 | H |
| 2-3-090 | N—Ph | N—Ph | b1 | H | b2 | H |
| 2-3-091 | N—Ph | N—Ph | b2 | H | b2 | H |
| 2-3-092 | N—Ph | N—Ph | b3 | H | b2 | H |
| 2-3-093 | N—Ph | N—Ph | H | H | b3 | H |
| 2-3-094 | N—Ph | N—Ph | t-bu | H | b3 | H |
| 2-3-095 | N—Ph | N—Ph | methyl | H | b3 | H |
| 2-3-096 | N—Ph | N—Ph | b1 | H | b3 | H |
| 2-3-097 | N—Ph | N—Ph | b2 | H | b3 | H |
| 2-3-098 | N—Ph | N—Ph | b3 | H | b3 | H |
| 2-3-099 | N—Ph | N—Ph | H | H | b4 | H |
| 2-3-100 | N—Ph | N—Ph | t-bu | H | b4 | H |
| 2-3-101 | N—Ph | N—Ph | methyl | H | b4 | H |
| 2-3-102 | N—Ph | N—Ph | b1 | H | b4 | H |
| 2-3-103 | N—Ph | N—Ph | b2 | H | b4 | H |
| 2-3-104 | N—Ph | N—Ph | b3 | H | b4 | H |
| 2-3-105 | N—Ph | N—Ph | H | H | b5 | H |
| 2-3-106 | N—Ph | N—Ph | t-bu | H | b5 | H |
| 2-3-107 | N—Ph | N—Ph | methyl | H | b5 | H |
| 2-3-108 | N—Ph | N—Ph | b1 | H | b5 | H |
| 2-3-109 | N—Ph | N—Ph | b2 | H | b5 | H |
| 2-3-110 | N—Ph | N—Ph | b3 | H | b5 | H |
| 2-3-111 | N—Ph | N—Ph | H | H | b6 | H |
| 2-3-112 | N—Ph | N—Ph | t-bu | H | b6 | H |
| 2-3-113 | N—Ph | N—Ph | methyl | H | b6 | H |
| 2-3-114 | N—Ph | N—Ph | b1 | H | b6 | H |
| 2-3-115 | N—Ph | N—Ph | b2 | H | b6 | H |
| 2-3-116 | N—Ph | N—Ph | b3 | H | b6 | H |
| 2-3-117 | N—Ph | N—Ph | H | H | b7 | H |
| 2-3-118 | N—Ph | N—Ph | t-bu | H | b7 | H |
| 2-3-119 | N—Ph | N—Ph | methyl | H | b7 | H |
| 2-3-120 | N—Ph | N—Ph | b1 | H | b7 | H |
| 2-3-121 | N—Ph | N—Ph | b2 | H | b7 | H |
| 2-3-122 | N—Ph | N—Ph | b3 | H | b7 | H |
| 2-3-123 | N—Ph | N—Ph | H | H | b8 | H |
| 2-3-124 | N—Ph | N—Ph | t-bu | H | b8 | H |
| 2-3-125 | N—Ph | N—Ph | methyl | H | b8 | H |
| 2-3-126 | N—Ph | N—Ph | b1 | H | b8 | H |
| 2-3-127 | N—Ph | N—Ph | b2 | H | b8 | H |
| 2-3-128 | N—Ph | N—Ph | b3 | H | b8 | H |
| 2-3-129 | N—Ph | N—Ph | H | H | b9 | H |
| 2-3-130 | N—Ph | N—Ph | t-bu | H | b9 | H |
| 2-3-131 | N—Ph | N—Ph | methyl | H | b9 | H |
| 2-3-132 | N—Ph | N—Ph | b1 | H | b9 | H |
| 2-3-133 | N—Ph | N—Ph | b2 | H | b9 | H |
| 2-3-134 | N—Ph | N—Ph | b3 | H | b9 | H |
| 2-3-135 | N—Ph | N—Ph | H | H | b10 | H |
| 2-3-136 | N—Ph | N—Ph | t-bu | H | b10 | H |
| 2-3-137 | N—Ph | N—Ph | methyl | H | b10 | H |
| 2-3-138 | N—Ph | N—Ph | b1 | H | b10 | H |
| 2-3-139 | N—Ph | N—Ph | b2 | H | b10 | H |
| 2-3-140 | N—Ph | N—Ph | b3 | H | b10 | H |
| 2-3-141 | N—Ph | N—Ph | H | H | a1 | a1 |
| 2-3-142 | N—Ph | N—Ph | t-bu | H | a1 | a1 |
| 2-3-143 | N—Ph | N—Ph | methyl | H | a1 | a1 |
| 2-3-144 | N—Ph | N—Ph | b1 | H | a1 | a1 |
| 2-3-145 | N—Ph | N—Ph | b2 | H | a1 | a1 |
| 2-3-146 | N—Ph | N—Ph | b3 | H | a1 | a1 |
| 2-3-147 | N—Ph | N—Ph | H | H | a2 | a2 |
| 2-3-148 | N—Ph | N—Ph | t-bu | H | a2 | a2 |
| 2-3-149 | N—Ph | N—Ph | methyl | H | a2 | a2 |
| 2-3-150 | N—Ph | N—Ph | b1 | H | a2 | a2 |
| 2-3-151 | N—Ph | N—Ph | b2 | H | a2 | a2 |
| 2-3-152 | N—Ph | N—Ph | b3 | H | a2 | a2 |
| 2-3-153 | N—Ph | N—Ph | H | H | a3 | a3 |
| 2-3-154 | N—Ph | N—Ph | t-bu | H | a3 | a3 |
| 2-3-155 | N—Ph | N—Ph | methyl | H | a3 | a3 |
| 2-3-156 | N—Ph | N—Ph | b1 | H | a3 | a3 |
| 2-3-157 | N—Ph | N—Ph | b2 | H | a3 | a3 |
| 2-3-158 | N—Ph | N—Ph | b3 | H | a3 | a3 |
| 2-3-159 | N—Ph | N—Ph | H | H | b1 | b1 |
| 2-3-160 | N—Ph | N—Ph | t-bu | H | b1 | b1 |
| 2-3-161 | N—Ph | N—Ph | methyl | H | b1 | b1 |
| 2-3-162 | N—Ph | N—Ph | b1 | H | b1 | b1 |
| 2-3-163 | N—Ph | N—Ph | b2 | H | b1 | b1 |
| 2-3-164 | N—Ph | N—Ph | b3 | H | b1 | b1 |
| 2-3-165 | N—Ph | N—Ph | H | H | b2 | b2 |
| 2-3-166 | N—Ph | N—Ph | t-bu | H | b2 | b2 |
| 2-3-167 | N—Ph | N—Ph | methyl | H | b2 | b2 |
| 2-3-168 | N—Ph | N—Ph | b1 | H | b2 | b2 |
| 2-3-169 | N—Ph | N—Ph | b2 | H | b2 | b2 |
| 2-3-170 | N—Ph | N—Ph | b3 | H | b2 | b2 |
| 2-3-171 | N—Ph | N—Ph | H | H | b3 | b3 |
| 2-3-172 | N—Ph | N—Ph | t-bu | H | b3 | b3 |
| 2-3-173 | N—Ph | N—Ph | methyl | H | b3 | b3 |
| 2-3-174 | N—Ph | N—Ph | b1 | H | b3 | b3 |
| 2-3-175 | N—Ph | N—Ph | b2 | H | b3 | b3 |
| 2-3-176 | N—Ph | N—Ph | b3 | H | b3 | b3 |
| 2-3-177 | N—Ph | N—Ph | H | H | b5 | b5 |
| 2-3-178 | N—Ph | N—Ph | t-bu | H | b5 | b5 |
| 2-3-179 | N—Ph | N—Ph | methyl | H | b5 | b5 |
| 2-3-180 | N—Ph | N—Ph | b1 | H | b5 | b5 |
| 2-3-181 | N—Ph | N—Ph | b2 | H | b5 | b5 |
| 2-3-182 | N—Ph | N—Ph | b3 | H | b5 | b5 |
| 2-3-183 | N—Ph | N—Ph | H | H | b6 | b6 |
| 2-3-184 | N—Ph | N—Ph | t-bu | H | b6 | b6 |
| 2-3-185 | N—Ph | N—Ph | methyl | H | b6 | b6 |
| 2-3-186 | N—Ph | N—Ph | b1 | H | b6 | b6 |
| 2-3-187 | N—Ph | N—Ph | b2 | H | b6 | b6 |
| 2-3-188 | N—Ph | N—Ph | b3 | H | b6 | b6 |
| 2-3-189 | N—Ph | N—Ph | H | H | b7 | b7 |
| 2-3-190 | N—Ph | N—Ph | t-bu | H | b7 | b7 |
| 2-3-191 | N—Ph | N—Ph | methyl | H | b7 | b7 |
| 2-3-192 | N—Ph | N—Ph | b1 | H | b7 | b7 |
| 2-3-193 | N—Ph | N—Ph | b2 | H | b7 | b7 |
| 2-3-194 | N—Ph | N—Ph | b3 | H | b7 | b7 |
| 2-3-195 | N-tolyl | N-tolyl | H | H | H | H |
| 2-3-196 | N-tolyl | N-tolyl | t-bu | H | H | H |
| 2-3-197 | N-tolyl | N-tolyl | methyl | H | H | H |
| 2-3-198 | N-tolyl | N-tolyl | t-bu | t-bu | t-bu | t-bu |
| 2-3-199 | N-tolyl | N-tolyl | methyl | methyl | methyl | methyl |
| 2-3-200 | N-tolyl | N-tolyl | b1 | H | H | H |
| 2-3-201 | N-tolyl | N-tolyl | b2 | H | H | H |
| 2-3-202 | N-tolyl | N-tolyl | b3 | H | H | H |
| 2-3-203 | N-tolyl | N-tolyl | H | H | H | a1 |
| 2-3-204 | N-tolyl | N-tolyl | t-bu | H | H | a1 |
| 2-3-205 | N-tolyl | N-tolyl | methyl | H | H | a1 |
| 2-3-206 | N-tolyl | N-tolyl | b1 | H | H | a1 |
| 2-3-207 | N-tolyl | N-tolyl | b2 | H | H | a1 |
| 2-3-208 | N-tolyl | N-tolyl | b3 | H | H | a1 |
| 2-3-209 | N-tolyl | N-tolyl | H | H | H | a2 |
| 2-3-210 | N-tolyl | N-tolyl | t-bu | H | H | a2 |
| 2-3-211 | N-tolyl | N-tolyl | methyl | H | H | a2 |
| 2-3-212 | N-tolyl | N-tolyl | b1 | H | H | a2 |
| 2-3-213 | N-tolyl | N-tolyl | b2 | H | H | a2 |
| 2-3-214 | N-tolyl | N-tolyl | b3 | H | H | a2 |
| 2-3-215 | N-tolyl | N-tolyl | H | H | H | a3 |
| 2-3-216 | N-tolyl | N-tolyl | t-bu | H | H | a3 |
| 2-3-217 | N-tolyl | N-tolyl | methyl | H | H | a3 |
| 2-3-218 | N-tolyl | N-tolyl | b1 | H | H | a3 |
| 2-3-219 | N-tolyl | N-tolyl | b2 | H | H | a3 |
| 2-3-220 | N-tolyl | N-tolyl | b3 | H | H | a3 |
| 2-3-221 | N-tolyl | N-tolyl | H | H | H | a4 |
| 2-3-222 | N-tolyl | N-tolyl | t-bu | H | H | a4 |
| 2-3-223 | N-tolyl | N-tolyl | methyl | H | H | a4 |
| 2-3-224 | N-tolyl | N-tolyl | b1 | H | H | a4 |
| 2-3-225 | N-tolyl | N-tolyl | b2 | H | H | a4 |
| 2-3-226 | N-tolyl | N-tolyl | b3 | H | H | a4 |
| 2-3-227 | N-tolyl | N-tolyl | H | H | H | a5 |
| 2-3-228 | N-tolyl | N-tolyl | t-bu | H | H | a5 |
| 2-3-229 | N-tolyl | N-tolyl | methyl | H | H | a5 |
| 2-3-230 | N-tolyl | N-tolyl | b1 | H | H | a5 |
| 2-3-231 | N-tolyl | N-tolyl | b2 | H | H | a5 |
| 2-3-232 | N-tolyl | N-tolyl | b3 | H | H | a5 |
| 2-3-233 | N-tolyl | N-tolyl | H | H | H | a6 |
| 2-3-234 | N-tolyl | N-tolyl | t-bu | H | H | a6 |
| 2-3-235 | N-tolyl | N-tolyl | methyl | H | H | a6 |
| 2-3-236 | N-tolyl | N-tolyl | b1 | H | H | a6 |
| 2-3-237 | N-tolyl | N-tolyl | b2 | H | H | a6 |
| 2-3-238 | N-tolyl | N-tolyl | b3 | H | H | a6 |
| 2-3-239 | N-tolyl | N-tolyl | H | H | a1 | H |
| 2-3-240 | N-tolyl | N-tolyl | t-bu | H | a1 | H |
| 2-3-241 | N-tolyl | N-tolyl | methyl | H | a1 | H |
| 2-3-242 | N-tolyl | N-tolyl | b1 | H | a1 | H |
| 2-3-243 | N-tolyl | N-tolyl | b2 | H | a1 | H |
| 2-3-244 | N-tolyl | N-tolyl | b3 | H | a1 | H |
| 2-3-245 | N-tolyl | N-tolyl | H | H | a2 | H |
| 2-3-246 | N-tolyl | N-tolyl | t-bu | H | a2 | H |
| 2-3-247 | N-tolyl | N-tolyl | methyl | H | a2 | H |
| 2-3-248 | N-tolyl | N-tolyl | b1 | H | a2 | H |
| 2-3-249 | N-tolyl | N-tolyl | b2 | H | a2 | H |
| 2-3-250 | N-tolyl | N-tolyl | b3 | H | a2 | H |
| 2-3-251 | N-tolyl | N-tolyl | H | H | a3 | H |
| 2-3-252 | N-tolyl | N-tolyl | t-bu | H | a3 | H |
| 2-3-253 | N-tolyl | N-tolyl | methyl | H | a3 | H |
| 2-3-254 | N-tolyl | N-tolyl | b1 | H | a3 | H |
| 2-3-255 | N-tolyl | N-tolyl | b2 | H | a3 | H |
| 2-3-256 | N-tolyl | N-tolyl | b3 | H | a3 | H |
| 2-3-257 | N-tolyl | N-tolyl | H | H | a4 | H |
| 2-3-258 | N-tolyl | N-tolyl | t-bu | H | a4 | H |
| 2-3-259 | N-tolyl | N-tolyl | methyl | H | a4 | H |
| 2-3-260 | N-tolyl | N-tolyl | b1 | H | a4 | H |
| 2-3-261 | N-tolyl | N-tolyl | b2 | H | a4 | H |
| 2-3-262 | N-tolyl | N-tolyl | b3 | H | a4 | H |
| 2-3-263 | N-tolyl | N-tolyl | H | H | a5 | H |
| 2-3-264 | N-tolyl | N-tolyl | t-bu | H | a5 | H |
| 2-3-265 | N-tolyl | N-tolyl | methyl | H | a5 | H |
| 2-3-266 | N-tolyl | N-tolyl | b1 | H | a5 | H |
| 2-3-267 | N-tolyl | N-tolyl | b2 | H | a5 | H |
| 2-3-268 | N-tolyl | N-tolyl | b3 | H | a5 | H |
| 2-3-269 | N-tolyl | N-tolyl | H | H | a6 | H |
| 2-3-270 | N-tolyl | N-tolyl | t-bu | H | a6 | H |
| 2-3-271 | N-tolyl | N-tolyl | methyl | H | a6 | H |
| 2-3-272 | N-tolyl | N-tolyl | b1 | H | a6 | H |
| 2-3-273 | N-tolyl | N-tolyl | b2 | H | a6 | H |
| 2-3-274 | N-tolyl | N-tolyl | b3 | H | a6 | H |
| 2-3-275 | N-tolyl | N-tolyl | H | H | b1 | H |
| 2-3-276 | N-tolyl | N-tolyl | t-bu | H | b1 | H |
| 2-3-277 | N-tolyl | N-tolyl | methyl | H | b1 | H |
| 2-3-278 | N-tolyl | N-tolyl | b1 | H | b1 | H |
| 2-3-279 | N-tolyl | N-tolyl | b2 | H | b1 | H |
| 2-3-280 | N-tolyl | N-tolyl | b3 | H | b1 | H |
| 2-3-281 | N-tolyl | N-tolyl | H | H | b2 | H |
| 2-3-282 | N-tolyl | N-tolyl | t-bu | H | b2 | H |
| 2-3-283 | N-tolyl | N-tolyl | methyl | H | b2 | H |
| 2-3-284 | N-tolyl | N-tolyl | b1 | H | b2 | H |
| 2-3-285 | N-tolyl | N-tolyl | b2 | H | b2 | H |
| 2-3-286 | N-tolyl | N-tolyl | b3 | H | b2 | H |
| 2-3-287 | N-tolyl | N-tolyl | H | H | b3 | H |
| 2-3-288 | N-tolyl | N-tolyl | t-bu | H | b3 | H |
| 2-3-289 | N-tolyl | N-tolyl | methyl | H | b3 | H |
| 2-3-290 | N-tolyl | N-tolyl | b1 | H | b3 | H |
| 2-3-291 | N-tolyl | N-tolyl | b2 | H | b3 | H |
| 2-3-292 | N-tolyl | N-tolyl | b3 | H | b3 | H |
| 2-3-293 | N-tolyl | N-tolyl | H | H | b4 | H |
| 2-3-294 | N-tolyl | N-tolyl | t-bu | H | b4 | H |
| 2-3-295 | N-tolyl | N-tolyl | methyl | H | b4 | H |
| 2-3-296 | N-tolyl | N-tolyl | b1 | H | b4 | H |
| 2-3-297 | N-tolyl | N-tolyl | b2 | H | b4 | H |
| 2-3-298 | N-tolyl | N-tolyl | b3 | H | b4 | H |
| 2-3-299 | N-tolyl | N-tolyl | H | H | b5 | H |
| 2-3-300 | N-tolyl | N-tolyl | t-bu | H | b5 | H |
| 2-3-301 | N-tolyl | N-tolyl | methyl | H | b5 | H |
| 2-3-302 | N-tolyl | N-tolyl | b1 | H | b5 | H |
| 2-3-303 | N-tolyl | N-tolyl | b2 | H | b5 | H |
| 2-3-304 | N-tolyl | N-tolyl | b3 | H | b5 | H |
| 2-3-305 | N-tolyl | N-tolyl | H | H | b6 | H |
| 2-3-306 | N-tolyl | N-tolyl | t-bu | H | b6 | H |
| 2-3-307 | N-tolyl | N-tolyl | methyl | H | b6 | H |
| 2-3-308 | N-tolyl | N-tolyl | b1 | H | b6 | H |
| 2-3-309 | N-tolyl | N-tolyl | b2 | H | b6 | H |
| 2-3-310 | N-tolyl | N-tolyl | b3 | H | b6 | H |
| 2-3-311 | N-tolyl | N-tolyl | H | H | b7 | H |
| 2-3-312 | N-tolyl | N-tolyl | t-bu | H | b7 | H |
| 2-3-313 | N-tolyl | N-tolyl | methyl | H | b7 | H |
| 2-3-314 | N-tolyl | N-tolyl | b1 | H | b7 | H |
| 2-3-315 | N-tolyl | N-tolyl | b2 | H | b7 | H |
| 2-3-316 | N-tolyl | N-tolyl | b3 | H | b7 | H |
| 2-3-317 | N-tolyl | N-tolyl | H | H | b8 | H |
| 2-3-318 | N-tolyl | N-tolyl | t-bu | H | b8 | H |
| 2-3-319 | N-tolyl | N-tolyl | methyl | H | b8 | H |
| 2-3-320 | N-tolyl | N-tolyl | b1 | H | b8 | H |
| 2-3-321 | N-tolyl | N-tolyl | b2 | H | b8 | H |
| 2-3-322 | N-tolyl | N-tolyl | b3 | H | b8 | H |
| 2-3-323 | N-tolyl | N-tolyl | H | H | b9 | H |
| 2-3-324 | N-tolyl | N-tolyl | t-bu | H | b9 | H |
| 2-3-325 | N-tolyl | N-tolyl | methyl | H | b9 | H |
| 2-3-326 | N-tolyl | N-tolyl | b1 | H | b9 | H |
| 2-3-327 | N-tolyl | N-tolyl | b2 | H | b9 | H |
| 2-3-328 | N-tolyl | N-tolyl | b3 | H | b9 | H |
| 2-3-329 | N-tolyl | N-tolyl | H | H | b10 | H |
| 2-3-330 | N-tolyl | N-tolyl | t-bu | H | b10 | H |
| 2-3-331 | N-tolyl | N-tolyl | methyl | H | b10 | H |
| 2-3-332 | N-tolyl | N-tolyl | b1 | H | b10 | H |
| 2-3-333 | N-tolyl | N-tolyl | b2 | H | b10 | H |
| 2-3-334 | N-tolyl | N-tolyl | b3 | H | b10 | H |
| 2-3-335 | N-tolyl | N-tolyl | H | H | a1 | a1 |
| 2-3-336 | N-tolyl | N-tolyl | t-bu | H | a1 | a1 |
| 2-3-337 | N-tolyl | N-tolyl | methyl | H | a1 | a1 |
| 2-3-338 | N-tolyl | N-tolyl | b1 | H | a1 | a1 |
| 2-3-339 | N-tolyl | N-tolyl | b2 | H | a1 | a1 |
| 2-3-340 | N-tolyl | N-tolyl | b3 | H | a1 | a1 |
| 2-3-341 | N-tolyl | N-tolyl | H | H | a2 | a2 |
| 2-3-342 | N-tolyl | N-tolyl | t-bu | H | a2 | a2 |
| 2-3-343 | N-tolyl | N-tolyl | methyl | H | a2 | a2 |
| 2-3-344 | N-tolyl | N-tolyl | b1 | H | a2 | a2 |
| 2-3-345 | N-tolyl | N-tolyl | b2 | H | a2 | a2 |
| 2-3-346 | N-tolyl | N-tolyl | b3 | H | a2 | a2 |
| 2-3-347 | N-tolyl | N-tolyl | H | H | a3 | a3 |
| 2-3-348 | N-tolyl | N-tolyl | t-bu | H | a3 | a3 |
| 2-3-349 | N-tolyl | N-tolyl | methyl | H | a3 | a3 |
| 2-3-350 | N-tolyl | N-tolyl | b1 | H | a3 | a3 |
| 2-3-351 | N-tolyl | N-tolyl | b2 | H | a3 | a3 |
| 2-3-352 | N-tolyl | N-tolyl | b3 | H | a3 | a3 |
| 2-3-353 | N-tolyl | N-tolyl | H | H | b1 | b1 |
| 2-3-354 | N-tolyl | N-tolyl | t-bu | H | b1 | b1 |
| 2-3-355 | N-tolyl | N-tolyl | methyl | H | b1 | b1 |
| 2-3-356 | N-tolyl | N-tolyl | b1 | H | b1 | b1 |
| 2-3-357 | N-tolyl | N-tolyl | b2 | H | b1 | b1 |
| 2-3-358 | N-tolyl | N-tolyl | b3 | H | b1 | b1 |
| 2-3-359 | N-tolyl | N-tolyl | H | H | b2 | b2 |
| 2-3-360 | N-tolyl | N-tolyl | t-bu | H | b2 | b2 |
| 2-3-361 | N-tolyl | N-tolyl | methyl | H | b2 | b2 |
| 2-3-362 | N-tolyl | N-tolyl | b1 | H | b2 | b2 |
| 2-3-363 | N-tolyl | N-tolyl | b2 | H | b2 | b2 |
| 2-3-364 | N-tolyl | N-tolyl | b3 | H | b2 | b2 |
| 2-3-365 | N-tolyl | N-tolyl | H | H | b3 | b3 |
| 2-3-366 | N-tolyl | N-tolyl | t-bu | H | b3 | b3 |
| 2-3-367 | N-tolyl | N-tolyl | methyl | H | b3 | b3 |
| 2-3-368 | N-tolyl | N-tolyl | b1 | H | b3 | b3 |
| 2-3-369 | N-tolyl | N-tolyl | b2 | H | b3 | b3 |
| 2-3-370 | N-tolyl | N-tolyl | b3 | H | b3 | b3 |
| 2-3-371 | N-tolyl | N-tolyl | H | H | b5 | b5 |
| 2-3-372 | N-tolyl | N-tolyl | t-bu | H | b5 | b5 |
| 2-3-373 | N-tolyl | N-tolyl | methyl | H | b5 | b5 |
| 2-3-374 | N-tolyl | N-tolyl | b1 | H | b5 | b5 |
| 2-3-375 | N-tolyl | N-tolyl | b2 | H | b5 | b5 |
| 2-3-376 | N-tolyl | N-tolyl | b3 | H | b5 | b5 |
| 2-3-377 | N-tolyl | N-tolyl | H | H | b6 | b6 |
| 2-3-378 | N-tolyl | N-tolyl | t-bu | H | b6 | b6 |
| 2-3-379 | N-tolyl | N-tolyl | methyl | H | b6 | b6 |
| 2-3-380 | N-tolyl | N-tolyl | b1 | H | b6 | b6 |
| 2-3-381 | N-tolyl | N-tolyl | b2 | H | b6 | b6 |
| 2-3-382 | N-tolyl | N-tolyl | b3 | H | b6 | b6 |
| 2-3-383 | N-tolyl | N-tolyl | H | H | b7 | b7 |
| 2-3-384 | N-tolyl | N-tolyl | t-bu | H | b7 | b7 |
| 2-3-385 | N-tolyl | N-tolyl | methyl | H | b7 | b7 |
| 2-3-386 | N-tolyl | N-tolyl | b1 | H | b7 | b7 |
| 2-3-387 | N-tolyl | N-tolyl | b2 | H | b7 | b7 |
| 2-3-388 | N-tolyl | N-tolyl | b3 | H | b7 | b7 |
| 2-3-389 | N-Biph | N-Biph | H | H | H | H |
| 2-3-390 | N-Biph | N-Biph | t-bu | H | H | H |
| 2-3-391 | N-Biph | N-Biph | methyl | H | H | H |
| 2-3-392 | N-Biph | N-Biph | t-bu | t-bu | t-bu | t-bu |
| 2-3-393 | N-Biph | N-Biph | methyl | methyl | methyl | methyl |
| 2-3-394 | N-Biph | N-Biph | b1 | H | H | H |
| 2-3-395 | N-Biph | N-Biph | b2 | H | H | H |
| 2-3-396 | N-Biph | N-Biph | b3 | H | H | H |
| 2-3-397 | N-Biph | N-Biph | H | H | H | a1 |
| 2-3-398 | N-Biph | N-Biph | t-bu | H | H | a1 |
| 2-3-399 | N-Biph | N-Biph | methyl | H | H | a1 |
| 2-3-400 | N-Biph | N-Biph | b1 | H | H | a1 |
| 2-3-401 | N-Biph | N-Biph | b2 | H | H | a1 |
| 2-3-402 | N-Biph | N-Biph | b3 | H | H | a1 |
| 2-3-403 | N-Biph | N-Biph | H | H | H | a2 |
| 2-3-404 | N-Biph | N-Biph | t-bu | H | H | a2 |
| 2-3-405 | N-Biph | N-Biph | methyl | H | H | a2 |
| 2-3-406 | N-Biph | N-Biph | b1 | H | H | a2 |
| 2-3-407 | N-Biph | N-Biph | b2 | H | H | a2 |
| 2-3-408 | N-Biph | N-Biph | b3 | H | H | a2 |
| 2-3-409 | N-Biph | N-Biph | H | H | H | a3 |
| 2-3-410 | N-Biph | N-Biph | t-bu | H | H | a3 |
| 2-3-411 | N-Biph | N-Biph | methyl | H | H | a3 |
| 2-3-412 | N-Biph | N-Biph | b1 | H | H | a3 |
| 2-3-413 | N-Biph | N-Biph | b2 | H | H | a3 |
| 2-3-414 | N-Biph | N-Biph | b3 | H | H | a3 |
| 2-3-415 | N-Biph | N-Biph | H | H | H | a4 |
| 2-3-416 | N-Biph | N-Biph | t-bu | H | H | a4 |
| 2-3-417 | N-Biph | N-Biph | methyl | H | H | a4 |
| 2-3-418 | N-Biph | N-Biph | b1 | H | H | a4 |
| 2-3-419 | N-Biph | N-Biph | b2 | H | H | a4 |
| 2-3-420 | N-Biph | N-Biph | b3 | H | H | a4 |
| 2-3-421 | N-Biph | N-Biph | H | H | H | a5 |
| 2-3-422 | N-Biph | N-Biph | t-bu | H | H | a5 |
| 2-3-423 | N-Biph | N-Biph | methyl | H | H | a5 |
| 2-3-424 | N-Biph | N-Biph | b1 | H | H | a5 |
| 2-3-425 | N-Biph | N-Biph | b2 | H | H | a5 |
| 2-3-426 | N-Biph | N-Biph | b3 | H | H | a5 |
| 2-3-427 | N-Biph | N-Biph | H | H | H | a6 |
| 2-3-428 | N-Biph | N-Biph | t-bu | H | H | a6 |
| 2-3-429 | N-Biph | N-Biph | methyl | H | H | a6 |
| 2-3-430 | N-Biph | N-Biph | b1 | H | H | a6 |
| 2-3-431 | N-Biph | N-Biph | b2 | H | H | a6 |
| 2-3-432 | N-Biph | N-Biph | b3 | H | H | a6 |
| 2-3-433 | N-Biph | N-Biph | H | H | a1 | H |
| 2-3-434 | N-Biph | N-Biph | t-bu | H | a1 | H |
| 2-3-435 | N-Biph | N-Biph | methyl | H | a1 | H |
| 2-3-436 | N-Biph | N-Biph | b1 | H | a1 | H |
| 2-3-437 | N-Biph | N-Biph | b2 | H | a1 | H |
| 2-3-438 | N-Biph | N-Biph | b3 | H | a1 | H |
| 2-3-439 | N-Biph | N-Biph | H | H | a2 | H |
| 2-3-440 | N-Biph | N-Biph | t-bu | H | a2 | H |
| 2-3-441 | N-Biph | N-Biph | methyl | H | a2 | H |
| 2-3-442 | N-Biph | N-Biph | b1 | H | a2 | H |
| 2-3-443 | N-Biph | N-Biph | b2 | H | a2 | H |
| 2-3-444 | N-Biph | N-Biph | b3 | H | a2 | H |
| 2-3-445 | N-Biph | N-Biph | H | H | a3 | H |
| 2-3-446 | N-Biph | N-Biph | t-bu | H | a3 | H |
| 2-3-447 | N-Biph | N-Biph | methyl | H | a3 | H |
| 2-3-448 | N-Biph | N-Biph | b1 | H | a3 | H |
| 2-3-449 | N-Biph | N-Biph | b2 | H | a3 | H |
| 2-3-450 | N-Biph | N-Biph | b3 | H | a3 | H |
| 2-3-451 | N-Biph | N-Biph | H | H | a4 | H |
| 2-3-452 | N-Biph | N-Biph | t-bu | H | a4 | H |
| 2-3-453 | N-Biph | N-Biph | methyl | H | a4 | H |
| 2-3-454 | N-Biph | N-Biph | b1 | H | a4 | H |
| 2-3-455 | N-Biph | N-Biph | b2 | H | a4 | H |
| 2-3-456 | N-Biph | N-Biph | b3 | H | a4 | H |
| 2-3-457 | N-Biph | N-Biph | H | H | a5 | H |
| 2-3-458 | N-Biph | N-Biph | t-bu | H | a5 | H |
| 2-3-459 | N-Biph | N-Biph | methyl | H | a5 | H |
| 2-3-460 | N-Biph | N-Biph | b1 | H | a5 | H |
| 2-3-461 | N-Biph | N-Biph | b2 | H | a5 | H |
| 2-3-462 | N-Biph | N-Biph | b3 | H | a5 | H |
| 2-3-463 | N-Biph | N-Biph | H | H | a6 | H |
| 2-3-464 | N-Biph | N-Biph | t-bu | H | a6 | H |
| 2-3-465 | N-Biph | N-Biph | methyl | H | a6 | H |
| 2-3-466 | N-Biph | N-Biph | b1 | H | a6 | H |
| 2-3-467 | N-Biph | N-Biph | b2 | H | a6 | H |
| 2-3-468 | N-Biph | N-Biph | b3 | H | a6 | H |
| 2-3-469 | N-Biph | N-Biph | H | H | b1 | H |
| 2-3-470 | N-Biph | N-Biph | t-bu | H | b1 | H |
| 2-3-471 | N-Biph | N-Biph | methyl | H | b1 | H |
| 2-3-472 | N-Biph | N-Biph | b1 | H | b1 | H |
| 2-3-473 | N-Biph | N-Biph | b2 | H | b1 | H |
| 2-3-474 | N-Biph | N-Biph | b3 | H | b1 | H |
| 2-3-475 | N-Biph | N-Biph | H | H | b2 | H |
| 2-3-476 | N-Biph | N-Biph | t-bu | H | b2 | H |
| 2-3-477 | N-Biph | N-Biph | methyl | H | b2 | H |
| 2-3-478 | N-Biph | N-Biph | b1 | H | b2 | H |
| 2-3-479 | N-Biph | N-Biph | b2 | H | b2 | H |
| 2-3-480 | N-Biph | N-Biph | b3 | H | b2 | H |
| 2-3-481 | N-Biph | N-Biph | H | H | b3 | H |
| 2-3-482 | N-Biph | N-Biph | t-bu | H | b3 | H |
| 2-3-483 | N-Biph | N-Biph | methyl | H | b3 | H |
| 2-3-484 | N-Biph | N-Biph | b1 | H | b3 | H |
| 2-3-485 | N-Biph | N-Biph | b2 | H | b3 | H |
| 2-3-486 | N-Biph | N-Biph | b3 | H | b3 | H |
| 2-3-487 | N-Biph | N-Biph | H | H | b4 | H |
| 2-3-488 | N-Biph | N-Biph | t-bu | H | b4 | H |
| 2-3-489 | N-Biph | N-Biph | methyl | H | b4 | H |
| 2-3-490 | N-Biph | N-Biph | b1 | H | b4 | H |
| 2-3-491 | N-Biph | N-Biph | b2 | H | b4 | H |
| 2-3-492 | N-Biph | N-Biph | b3 | H | b4 | H |
| 2-3-493 | N-Biph | N-Biph | H | H | b5 | H |
| 2-3-494 | N-Biph | N-Biph | t-bu | H | b5 | H |
| 2-3-495 | N-Biph | N-Biph | methyl | H | b5 | H |
| 2-3-496 | N-Biph | N-Biph | b1 | H | b5 | H |
| 2-3-497 | N-Biph | N-Biph | b2 | H | b5 | H |
| 2-3-498 | N-Biph | N-Biph | b3 | H | b5 | H |
| 2-3-499 | N-Biph | N-Biph | H | H | b6 | H |
| 2-3-500 | N-Biph | N-Biph | t-bu | H | b6 | H |
| 2-3-501 | N-Biph | N-Biph | methyl | H | b6 | H |
| 2-3-502 | N-Biph | N-Biph | b1 | H | b6 | H |
| 2-3-503 | N-Biph | N-Biph | b2 | H | b6 | H |
| 2-3-504 | N-Biph | N-Biph | b3 | H | b6 | H |
| 2-3-505 | N-Biph | N-Biph | H | H | b7 | H |
| 2-3-506 | N-Biph | N-Biph | t-bu | H | b7 | H |
| 2-3-507 | N-Biph | N-Biph | methyl | H | b7 | H |
| 2-3-508 | N-Biph | N-Biph | b1 | H | b7 | H |
| 2-3-509 | N-Biph | N-Biph | b2 | H | b7 | H |
| 2-3-510 | N-Biph | N-Biph | b3 | H | b7 | H |
| 2-3-511 | N-Biph | N-Biph | H | H | b8 | H |
| 2-3-512 | N-Biph | N-Biph | t-bu | H | b8 | H |
| 2-3-513 | N-Biph | N-Biph | methyl | H | b8 | H |
| 2-3-514 | N-Biph | N-Biph | b1 | H | b8 | H |
| 2-3-515 | N-Biph | N-Biph | b2 | H | b8 | H |
| 2-3-516 | N-Biph | N-Biph | b3 | H | b8 | H |
| 2-3-517 | N-Biph | N-Biph | H | H | b9 | H |
| 2-3-518 | N-Biph | N-Biph | t-bu | H | b9 | H |
| 2-3-519 | N-Biph | N-Biph | methyl | H | b9 | H |
| 2-3-520 | N-Biph | N-Biph | b1 | H | b9 | H |
| 2-3-521 | N-Biph | N-Biph | b2 | H | b9 | H |
| 2-3-522 | N-Biph | N-Biph | b3 | H | b9 | H |
| 2-3-523 | N-Biph | N-Biph | H | H | b10 | H |
| 2-3-524 | N-Biph | N-Biph | t-bu | H | b10 | H |
| 2-3-525 | N-Biph | N-Biph | methyl | H | b10 | H |
| 2-3-526 | N-Biph | N-Biph | b1 | H | b10 | H |
| 2-3-527 | N-Biph | N-Biph | b2 | H | b10 | H |
| 2-3-528 | N-Biph | N-Biph | b3 | H | b10 | H |
| 2-3-529 | N-Biph | N-Biph | H | H | a1 | a1 |
| 2-3-530 | N-Biph | N-Biph | t-bu | H | a1 | a1 |
| 2-3-531 | N-Biph | N-Biph | methyl | H | a1 | a1 |
| 2-3-532 | N-Biph | N-Biph | b1 | H | a1 | a1 |
| 2-3-533 | N-Biph | N-Biph | b2 | H | a1 | a1 |
| 2-3-534 | N-Biph | N-Biph | b3 | H | a1 | a1 |
| 2-3-535 | N-Biph | N-Biph | H | H | a2 | a2 |
| 2-3-536 | N-Biph | N-Biph | t-bu | H | a2 | a2 |
| 2-3-537 | N-Biph | N-Biph | methyl | H | a2 | a2 |
| 2-3-538 | N-Biph | N-Biph | b1 | H | a2 | a2 |
| 2-3-539 | N-Biph | N-Biph | b2 | H | a2 | a2 |
| 2-3-540 | N-Biph | N-Biph | b3 | H | a2 | a2 |
| 2-3-541 | N-Biph | N-Biph | H | H | a3 | a3 |
| 2-3-542 | N-Biph | N-Biph | t-bu | H | a3 | a3 |
| 2-3-543 | N-Biph | N-Biph | methyl | H | a3 | a3 |
| 2-3-544 | N-Biph | N-Biph | b1 | H | a3 | a3 |
| 2-3-545 | N-Biph | N-Biph | b2 | H | a3 | a3 |
| 2-3-546 | N-Biph | N-Biph | b3 | H | a3 | a3 |
| 2-3-547 | N-Biph | N-Biph | H | H | b1 | b1 |
| 2-3-548 | N-Biph | N-Biph | t-bu | H | b1 | b1 |
| 2-3-549 | N-Biph | N-Biph | methyl | H | b1 | b1 |
| 2-3-550 | N-Biph | N-Biph | b1 | H | b1 | b1 |
| 2-3-551 | N-Biph | N-Biph | b2 | H | b1 | b1 |
| 2-3-552 | N-Biph | N-Biph | b3 | H | b1 | b1 |
| 2-3-553 | N-Biph | N-Biph | H | H | b2 | b2 |
| 2-3-554 | N-Biph | N-Biph | t-bu | H | b2 | b2 |
| 2-3-555 | N-Biph | N-Biph | methyl | H | b2 | b2 |
| 2-3-556 | N-Biph | N-Biph | b1 | H | b2 | b2 |
| 2-3-557 | N-Biph | N-Biph | b2 | H | b2 | b2 |
| 2-3-558 | N-Biph | N-Biph | b3 | H | b2 | b2 |
| 2-3-559 | N-Biph | N-Biph | H | H | b3 | b3 |
| 2-3-560 | N-Biph | N-Biph | t-bu | H | b3 | b3 |
| 2-3-561 | N-Biph | N-Biph | methyl | H | b3 | b3 |
| 2-3-562 | N-Biph | N-Biph | b1 | H | b3 | b3 |
| 2-3-563 | N-Biph | N-Biph | b2 | H | b3 | b3 |
| 2-3-564 | N-Biph | N-Biph | b3 | H | b3 | b3 |
| 2-3-565 | N-Biph | N-Biph | H | H | b5 | b5 |
| 2-3-566 | N-Biph | N-Biph | t-bu | H | b5 | b5 |
| 2-3-567 | N-Biph | N-Biph | methyl | H | b5 | b5 |
| 2-3-568 | N-Biph | N-Biph | b1 | H | b5 | b5 |
| 2-3-569 | N-Biph | N-Biph | b2 | H | b5 | b5 |
| 2-3-570 | N-Biph | N-Biph | b3 | H | b5 | b5 |
| 2-3-571 | N-Biph | N-Biph | H | H | b6 | b6 |
| 2-3-572 | N-Biph | N-Biph | t-bu | H | b6 | b6 |
| 2-3-573 | N-Biph | N-Biph | methyl | H | b6 | b6 |
| 2-3-574 | N-Biph | N-Biph | b1 | H | b6 | b6 |
| 2-3-575 | N-Biph | N-Biph | b2 | H | b6 | b6 |
| 2-3-576 | N-Biph | N-Biph | b3 | H | b6 | b6 |
| 2-3-577 | N-Biph | N-Biph | H | H | b7 | b7 |
| 2-3-578 | N-Biph | N-Biph | t-bu | H | b7 | b7 |
| 2-3-579 | N-Biph | N-Biph | methyl | H | b7 | b7 |
| 2-3-580 | N-Biph | N-Biph | b1 | H | b7 | b7 |
| 2-3-581 | N-Biph | N-Biph | b2 | H | b7 | b7 |
| 2-3-582 | N-Biph | N-Biph | b3 | H | b7 | b7 |
| 2-3-583 | O | N—Ph | H | H | H | H |
| 2-3-584 | O | N—Ph | t-bu | H | H | H |
| 2-3-585 | O | N—Ph | methyl | H | H | H |
| 2-3-586 | O | N—Ph | t-bu | t-bu | t-bu | t-bu |
| 2-3-587 | O | N—Ph | methyl | methyl | methyl | methyl |
| 2-3-588 | O | N—Ph | b1 | H | H | H |
| 2-3-589 | O | N—Ph | b2 | H | H | H |
| 2-3-590 | O | N—Ph | b3 | H | H | H |
| 2-3-591 | O | N—Ph | H | H | H | a1 |
| 2-3-592 | O | N—Ph | t-bu | H | H | a1 |
| 2-3-593 | O | N—Ph | methyl | H | H | a1 |
| 2-3-594 | O | N—Ph | b1 | H | H | a1 |
| 2-3-595 | O | N—Ph | b2 | H | H | a1 |
| 2-3-596 | O | N—Ph | b3 | H | H | a1 |
| 2-3-597 | O | N—Ph | H | H | H | a2 |
| 2-3-598 | O | N—Ph | t-bu | H | H | a2 |
| 2-3-599 | O | N—Ph | methyl | H | H | a2 |
| 2-3-600 | O | N—Ph | b1 | H | H | a2 |
| 2-3-601 | O | N—Ph | b2 | H | H | a2 |
| 2-3-602 | O | N—Ph | b3 | H | H | a2 |
| 2-3-603 | O | N—Ph | H | H | H | a3 |
| 2-3-604 | O | N—Ph | t-bu | H | H | a3 |
| 2-3-605 | O | N—Ph | methyl | H | H | a3 |
| 2-3-606 | O | N—Ph | b1 | H | H | a3 |
| 2-3-607 | O | N—Ph | b2 | H | H | a3 |
| 2-3-608 | O | N—Ph | b3 | H | H | a3 |
| 2-3-609 | O | N—Ph | H | H | H | a4 |
| 2-3-610 | O | N—Ph | t-bu | H | H | a4 |
| 2-3-611 | O | N—Ph | methyl | H | H | a4 |
| 2-3-612 | O | N—Ph | b1 | H | H | a4 |
| 2-3-613 | O | N—Ph | b2 | H | H | a4 |
| 2-3-614 | O | N—Ph | b3 | H | H | a4 |
| 2-3-615 | O | N—Ph | H | H | H | a5 |
| 2-3-616 | O | N—Ph | t-bu | H | H | a5 |
| 2-3-617 | O | N—Ph | methyl | H | H | a5 |
| 2-3-618 | O | N—Ph | b1 | H | H | a5 |
| 2-3-619 | O | N—Ph | b2 | H | H | a5 |
| 2-3-620 | O | N—Ph | b3 | H | H | a5 |
| 2-3-621 | O | N—Ph | H | H | H | a6 |
| 2-3-622 | O | N—Ph | t-bu | H | H | a6 |
| 2-3-623 | O | N—Ph | methyl | H | H | a6 |
| 2-3-624 | O | N—Ph | b1 | H | H | a6 |
| 2-3-625 | O | N—Ph | b2 | H | H | a6 |
| 2-3-626 | O | N—Ph | b3 | H | H | a6 |
| 2-3-627 | O | N—Ph | H | H | a1 | H |
| 2-3-628 | O | N—Ph | t-bu | H | a1 | H |
| 2-3-629 | O | N—Ph | methyl | H | a1 | H |
| 2-3-630 | O | N—Ph | b1 | H | a1 | H |
| 2-3-631 | O | N—Ph | b2 | H | a1 | H |
| 2-3-632 | O | N—Ph | b3 | H | a1 | H |
| 2-3-633 | O | N—Ph | H | H | a2 | H |
| 2-3-634 | O | N—Ph | t-bu | H | a2 | H |
| 2-3-635 | O | N—Ph | methyl | H | a2 | H |
| 2-3-636 | O | N—Ph | b1 | H | a2 | H |
| 2-3-637 | O | N—Ph | b2 | H | a2 | H |
| 2-3-638 | O | N—Ph | b3 | H | a2 | H |
| 2-3-639 | O | N—Ph | H | H | a3 | H |
| 2-3-640 | O | N—Ph | t-bu | H | a3 | H |
| 2-3-641 | O | N—Ph | methyl | H | a3 | H |
| 2-3-642 | O | N—Ph | b1 | H | a3 | H |
| 2-3-643 | O | N—Ph | b2 | H | a3 | H |
| 2-3-644 | O | N—Ph | b3 | H | a3 | H |
| 2-3-645 | O | N—Ph | H | H | a4 | H |
| 2-3-646 | O | N—Ph | t-bu | H | a4 | H |
| 2-3-647 | O | N—Ph | methyl | H | a4 | H |
| 2-3-648 | O | N—Ph | b1 | H | a4 | H |
| 2-3-649 | O | N—Ph | b2 | H | a4 | H |
| 2-3-650 | O | N—Ph | b3 | H | a4 | H |
| 2-3-651 | O | N—Ph | H | H | a5 | H |
| 2-3-652 | O | N—Ph | t-bu | H | a5 | H |
| 2-3-653 | O | N—Ph | methyl | H | a5 | H |
| 2-3-654 | O | N—Ph | b1 | H | a5 | H |
| 2-3-655 | O | N—Ph | b2 | H | a5 | H |
| 2-3-656 | O | N—Ph | b3 | H | a5 | H |
| 2-3-657 | O | N—Ph | H | H | a6 | H |
| 2-3-658 | O | N—Ph | t-bu | H | a6 | H |
| 2-3-659 | O | N—Ph | methyl | H | a6 | H |
| 2-3-660 | O | N—Ph | b1 | H | a6 | H |
| 2-3-661 | O | N—Ph | b2 | H | a6 | H |
| 2-3-662 | O | N—Ph | b3 | H | a6 | H |
| 2-3-663 | O | N—Ph | H | H | b1 | H |
| 2-3-664 | O | N—Ph | t-bu | H | b1 | H |
| 2-3-665 | O | N—Ph | methyl | H | b1 | H |
| 2-3-666 | O | N—Ph | b1 | H | b1 | H |
| 2-3-667 | O | N—Ph | b2 | H | b1 | H |
| 2-3-668 | O | N—Ph | b3 | H | b1 | H |
| 2-3-669 | O | N—Ph | H | H | b2 | H |
| 2-3-670 | O | N—Ph | t-bu | H | b2 | H |
| 2-3-671 | O | N—Ph | methyl | H | b2 | H |
| 2-3-672 | O | N—Ph | b1 | H | b2 | H |
| 2-3-673 | O | N—Ph | b2 | H | b2 | H |
| 2-3-674 | O | N—Ph | b3 | H | b2 | H |
| 2-3-675 | O | N—Ph | H | H | b3 | H |
| 2-3-676 | O | N—Ph | t-bu | H | b3 | H |
| 2-3-677 | O | N—Ph | methyl | H | b3 | H |
| 2-3-678 | O | N—Ph | b1 | H | b3 | H |
| 2-3-679 | O | N—Ph | b2 | H | b3 | H |
| 2-3-680 | O | N—Ph | b3 | H | b3 | H |
| 2-3-681 | O | N—Ph | H | H | b4 | H |
| 2-3-682 | O | N—Ph | t-bu | H | b4 | H |
| 2-3-683 | O | N—Ph | methyl | H | b4 | H |
| 2-3-684 | O | N—Ph | b1 | H | b4 | H |
| 2-3-685 | O | N—Ph | b2 | H | b4 | H |
| 2-3-686 | O | N—Ph | b3 | H | b4 | H |
| 2-3-687 | O | N—Ph | H | H | b5 | H |
| 2-3-688 | O | N—Ph | t-bu | H | b5 | H |
| 2-3-689 | O | N—Ph | methyl | H | b5 | H |
| 2-3-690 | O | N—Ph | b1 | H | b5 | H |
| 2-3-691 | O | N—Ph | b2 | H | b5 | H |
| 2-3-692 | O | N—Ph | b3 | H | b5 | H |
| 2-3-693 | O | N—Ph | H | H | b6 | H |
| 2-3-694 | O | N—Ph | t-bu | H | b6 | H |
| 2-3-695 | O | N—Ph | methyl | H | b6 | H |
| 2-3-696 | O | N—Ph | b1 | H | b6 | H |
| 2-3-697 | O | N—Ph | b2 | H | b6 | H |
| 2-3-698 | O | N—Ph | b3 | H | b6 | H |
| 2-3-699 | O | N—Ph | H | H | b7 | H |
| 2-3-700 | O | N—Ph | t-bu | H | b7 | H |
| 2-3-701 | O | N—Ph | methyl | H | b7 | H |
| 2-3-702 | O | N—Ph | b1 | H | b7 | H |
| 2-3-703 | O | N—Ph | b2 | H | b7 | H |
| 2-3-704 | O | N—Ph | b3 | H | b7 | H |
| 2-3-705 | O | N—Ph | H | H | b8 | H |
| 2-3-706 | O | N—Ph | t-bu | H | b8 | H |
| 2-3-707 | O | N—Ph | methyl | H | b8 | H |
| 2-3-708 | O | N—Ph | b1 | H | b8 | H |
| 2-3-709 | O | N—Ph | b2 | H | b8 | H |
| 2-3-710 | O | N—Ph | b3 | H | b8 | H |
| 2-3-711 | O | N—Ph | H | H | b9 | H |
| 2-3-712 | O | N—Ph | t-bu | H | b9 | H |
| 2-3-713 | O | N—Ph | methyl | H | b9 | H |
| 2-3-714 | O | N—Ph | b1 | H | b9 | H |
| 2-3-715 | O | N—Ph | b2 | H | b9 | H |
| 2-3-716 | O | N—Ph | b3 | H | b9 | H |
| 2-3-717 | O | N—Ph | H | H | b10 | H |
| 2-3-718 | O | N—Ph | t-bu | H | b10 | H |
| 2-3-719 | O | N—Ph | methyl | H | b10 | H |
| 2-3-720 | O | N—Ph | b1 | H | b10 | H |
| 2-3-721 | O | N—Ph | b2 | H | b10 | H |
| 2-3-722 | O | N—Ph | b3 | H | b10 | H |
| 2-3-723 | O | N—Ph | H | H | a1 | a1 |
| 2-3-724 | O | N—Ph | t-bu | H | a1 | a1 |
| 2-3-725 | O | N—Ph | methyl | H | a1 | a1 |
| 2-3-726 | O | N—Ph | b1 | H | a1 | a1 |
| 2-3-727 | O | N—Ph | b2 | H | a1 | a1 |
| 2-3-728 | O | N—Ph | b3 | H | a1 | a1 |
| 2-3-729 | O | N—Ph | H | H | a2 | a2 |
| 2-3-730 | O | N—Ph | t-bu | H | a2 | a2 |
| 2-3-731 | O | N—Ph | methyl | H | a2 | a2 |
| 2-3-732 | O | N—Ph | b1 | H | a2 | a2 |
| 2-3-733 | O | N—Ph | b2 | H | a2 | a2 |
| 2-3-734 | O | N—Ph | b3 | H | a2 | a2 |
| 2-3-735 | O | N—Ph | H | H | a3 | a3 |
| 2-3-736 | O | N—Ph | t-bu | H | a3 | a3 |
| 2-3-737 | O | N—Ph | methyl | H | a3 | a3 |
| 2-3-738 | O | N—Ph | b1 | H | a3 | a3 |
| 2-3-739 | O | N—Ph | b2 | H | a3 | a3 |
| 2-3-740 | O | N—Ph | b3 | H | a3 | a3 |
| 2-3-741 | O | N—Ph | H | H | b1 | b1 |
| 2-3-742 | O | N—Ph | t-bu | H | b1 | b1 |
| 2-3-743 | O | N—Ph | methyl | H | b1 | b1 |
| 2-3-744 | O | N—Ph | b1 | H | b1 | b1 |
| 2-3-745 | O | N—Ph | b2 | H | b1 | b1 |
| 2-3-746 | O | N—Ph | b3 | H | b1 | b1 |
| 2-3-747 | O | N—Ph | H | H | b2 | b2 |
| 2-3-748 | O | N—Ph | t-bu | H | b2 | b2 |
| 2-3-749 | O | N—Ph | methyl | H | b2 | b2 |
| 2-3-750 | O | N—Ph | b1 | H | b2 | b2 |
| 2-3-751 | O | N—Ph | b2 | H | b2 | b2 |
| 2-3-752 | O | N—Ph | b3 | H | b2 | b2 |
| 2-3-753 | O | N—Ph | H | H | b3 | b3 |
| 2-3-754 | O | N—Ph | t-bu | H | b3 | b3 |
| 2-3-755 | O | N—Ph | methyl | H | b3 | b3 |
| 2-3-756 | O | N—Ph | b1 | H | b3 | b3 |
| 2-3-757 | O | N—Ph | b2 | H | b3 | b3 |
| 2-3-758 | O | N—Ph | b3 | H | b3 | b3 |
| 2-3-759 | O | N—Ph | H | H | b5 | b5 |
| 2-3-760 | O | N—Ph | t-bu | H | b5 | b5 |
| 2-3-761 | O | N—Ph | methyl | H | b5 | b5 |
| 2-3-762 | O | N—Ph | b1 | H | b5 | b5 |
| 2-3-763 | O | N—Ph | b2 | H | b5 | b5 |
| 2-3-764 | O | N—Ph | b3 | H | b5 | b5 |
| 2-3-765 | O | N—Ph | H | H | b6 | b6 |
| 2-3-766 | O | N—Ph | t-bu | H | b6 | b6 |
| 2-3-767 | O | N—Ph | methyl | H | b6 | b6 |
| 2-3-768 | O | N—Ph | b1 | H | b6 | b6 |
| 2-3-769 | O | N—Ph | b2 | H | b6 | b6 |
| 2-3-770 | O | N—Ph | b3 | H | b6 | b6 |
| 2-3-771 | O | N—Ph | H | H | b7 | b7 |
| 2-3-772 | O | N—Ph | t-bu | H | b7 | b7 |
| 2-3-773 | O | N—Ph | methyl | H | b7 | b7 |
| 2-3-774 | O | N—Ph | b1 | H | b7 | b7 |
| 2-3-775 | O | N—Ph | b2 | H | b7 | b7 |
| 2-3-776 | O | N—Ph | b3 | H | b7 | b7 |
| 2-3-777 | S | N—Ph | H | H | H | H |
| 2-3-778 | S | N—Ph | t-bu | H | H | H |
| 2-3-779 | S | N—Ph | methyl | H | H | H |
| 2-3-780 | S | N—Ph | t-bu | t-bu | t-bu | t-bu |
| 2-3-781 | S | N—Ph | methyl | methyl | methyl | methyl |
| 2-3-782 | S | N—Ph | b1 | H | H | H |
| 2-3-783 | S | N—Ph | b2 | H | H | H |
| 2-3-784 | S | N—Ph | b3 | H | H | H |
| 2-3-785 | S | N—Ph | H | H | H | a1 |
| 2-3-786 | S | N—Ph | t-bu | H | H | a1 |
| 2-3-787 | S | N—Ph | methyl | H | H | a1 |
| 2-3-788 | S | N—Ph | b1 | H | H | a1 |
| 2-3-789 | S | N—Ph | b2 | H | H | a1 |
| 2-3-790 | S | N—Ph | b3 | H | H | a1 |
| 2-3-791 | S | N—Ph | H | H | H | a2 |
| 2-3-792 | S | N—Ph | t-bu | H | H | a2 |
| 2-3-793 | S | N—Ph | methyl | H | H | a2 |
| 2-3-794 | S | N—Ph | b1 | H | H | a2 |
| 2-3-795 | S | N—Ph | b2 | H | H | a2 |
| 2-3-796 | S | N—Ph | b3 | H | H | a2 |
| 2-3-797 | S | N—Ph | H | H | H | a3 |
| 2-3-798 | S | N—Ph | t-bu | H | H | a3 |
| 2-3-799 | S | N—Ph | methyl | H | H | a3 |
| 2-3-800 | S | N—Ph | b1 | H | H | a3 |
| 2-3-801 | S | N—Ph | b2 | H | H | a3 |
| 2-3-802 | S | N—Ph | b3 | H | H | a3 |
| 2-3-803 | S | N—Ph | H | H | H | a4 |
| 2-3-804 | S | N—Ph | t-bu | H | H | a4 |
| 2-3-805 | S | N—Ph | methyl | H | H | a4 |
| 2-3-806 | S | N—Ph | b1 | H | H | a4 |
| 2-3-807 | S | N—Ph | b2 | H | H | a4 |
| 2-3-808 | S | N—Ph | b3 | H | H | a4 |
| 2-3-809 | S | N—Ph | H | H | H | a5 |
| 2-3-810 | S | N—Ph | t-bu | H | H | a5 |
| 2-3-811 | S | N—Ph | methyl | H | H | a5 |
| 2-3-812 | S | N—Ph | b1 | H | H | a5 |
| 2-3-813 | S | N—Ph | b2 | H | H | a5 |
| 2-3-814 | S | N—Ph | b3 | H | H | a5 |
| 2-3-815 | S | N—Ph | H | H | H | a6 |
| 2-3-816 | S | N—Ph | t-bu | H | H | a6 |
| 2-3-817 | S | N—Ph | methyl | H | H | a6 |
| 2-3-818 | S | N—Ph | b1 | H | H | a6 |
| 2-3-819 | S | N—Ph | b2 | H | H | a6 |
| 2-3-820 | S | N—Ph | b3 | H | H | a6 |
| 2-3-821 | S | N—Ph | H | H | a1 | H |
| 2-3-822 | S | N—Ph | t-bu | H | a1 | H |
| 2-3-823 | S | N—Ph | methyl | H | a1 | H |
| 2-3-824 | S | N—Ph | b1 | H | a1 | H |
| 2-3-825 | S | N—Ph | b2 | H | a1 | H |
| 2-3-826 | S | N—Ph | b3 | H | a1 | H |
| 2-3-827 | S | N—Ph | H | H | a2 | H |
| 2-3-828 | S | N—Ph | t-bu | H | a2 | H |
| 2-3-829 | S | N—Ph | methyl | H | a2 | H |
| 2-3-830 | S | N—Ph | b1 | H | a2 | H |
| 2-3-831 | S | N—Ph | b2 | H | a2 | H |
| 2-3-832 | S | N—Ph | b3 | H | a2 | H |
| 2-3-833 | S | N—Ph | H | H | a3 | H |
| 2-3-834 | S | N—Ph | t-bu | H | a3 | H |
| 2-3-835 | S | N—Ph | methyl | H | a3 | H |
| 2-3-836 | S | N—Ph | b1 | H | a3 | H |
| 2-3-837 | S | N—Ph | b2 | H | a3 | H |
| 2-3-838 | S | N—Ph | b3 | H | a3 | H |
| 2-3-839 | S | N—Ph | H | H | a4 | H |
| 2-3-840 | S | N—Ph | t-bu | H | a4 | H |
| 2-3-841 | S | N—Ph | methyl | H | a4 | H |
| 2-3-842 | S | N—Ph | b1 | H | a4 | H |
| 2-3-843 | S | N—Ph | b2 | H | a4 | H |
| 2-3-844 | S | N—Ph | b3 | H | a4 | H |
| 2-3-845 | S | N—Ph | H | H | a5 | H |
| 2-3-846 | S | N—Ph | t-bu | H | a5 | H |
| 2-3-847 | S | N—Ph | methyl | H | a5 | H |
| 2-3-848 | S | N—Ph | b1 | H | a5 | H |
| 2-3-849 | S | N—Ph | b2 | H | a5 | H |
| 2-3-850 | S | N—Ph | b3 | H | a5 | H |
| 2-3-851 | S | N—Ph | H | H | a6 | H |
| 2-3-852 | S | N—Ph | t-bu | H | a6 | H |
| 2-3-853 | S | N—Ph | methyl | H | a6 | H |
| 2-3-854 | S | N—Ph | b1 | H | a6 | H |
| 2-3-855 | S | N—Ph | b2 | H | a6 | H |
| 2-3-856 | S | N—Ph | b3 | H | a6 | H |
| 2-3-857 | S | N—Ph | H | H | b1 | H |
| 2-3-858 | S | N—Ph | t-bu | H | b1 | H |
| 2-3-859 | S | N—Ph | methyl | H | b1 | H |
| 2-3-860 | S | N—Ph | b1 | H | b1 | H |
| 2-3-861 | S | N—Ph | b2 | H | b1 | H |
| 2-3-862 | S | N—Ph | b3 | H | b1 | H |
| 2-3-863 | S | N—Ph | H | H | b2 | H |
| 2-3-864 | S | N—Ph | t-bu | H | b2 | H |
| 2-3-865 | S | N—Ph | methyl | H | b2 | H |
| 2-3-866 | S | N—Ph | b1 | H | b2 | H |
| 2-3-867 | S | N—Ph | b2 | H | b2 | H |
| 2-3-868 | S | N—Ph | b3 | H | b2 | H |
| 2-3-869 | S | N—Ph | H | H | b3 | H |
| 2-3-870 | S | N—Ph | t-bu | H | b3 | H |
| 2-3-871 | S | N—Ph | methyl | H | b3 | H |
| 2-3-872 | S | N—Ph | b1 | H | b3 | H |
| 2-3-873 | S | N—Ph | b2 | H | b3 | H |
| 2-3-874 | S | N—Ph | b3 | H | b3 | H |
| 2-3-875 | S | N—Ph | H | H | b4 | H |
| 2-3-876 | S | N—Ph | t-bu | H | b4 | H |
| 2-3-877 | S | N—Ph | methyl | H | b4 | H |
| 2-3-878 | S | N—Ph | b1 | H | b4 | H |
| 2-3-879 | S | N—Ph | b2 | H | b4 | H |
| 2-3-880 | S | N—Ph | b3 | H | b4 | H |
| 2-3-881 | S | N—Ph | H | H | b5 | H |
| 2-3-882 | S | N—Ph | t-bu | H | b5 | H |
| 2-3-883 | S | N—Ph | methyl | H | b5 | H |
| 2-3-884 | S | N—Ph | b1 | H | b5 | H |
| 2-3-885 | S | N—Ph | b2 | H | b5 | H |
| 2-3-886 | S | N—Ph | b3 | H | b5 | H |
| 2-3-887 | S | N—Ph | H | H | b6 | H |
| 2-3-888 | S | N—Ph | t-bu | H | b6 | H |
| 2-3-889 | S | N—Ph | methyl | H | b6 | H |
| 2-3-890 | S | N—Ph | b1 | H | b6 | H |
| 2-3-891 | S | N—Ph | b2 | H | b6 | H |
| 2-3-892 | S | N—Ph | b3 | H | b6 | H |
| 2-3-893 | S | N—Ph | H | H | b7 | H |
| 2-3-894 | S | N—Ph | t-bu | H | b7 | H |
| 2-3-895 | S | N—Ph | methyl | H | b7 | H |
| 2-3-896 | S | N—Ph | b1 | H | b7 | H |
| 2-3-897 | S | N—Ph | b2 | H | b7 | H |
| 2-3-898 | S | N—Ph | b3 | H | b7 | H |
| 2-3-899 | S | N—Ph | H | H | b8 | H |
| 2-3-900 | S | N—Ph | t-bu | H | b8 | H |
| 2-3-901 | S | N—Ph | methyl | H | b8 | H |
| 2-3-902 | S | N—Ph | b1 | H | b8 | H |
| 2-3-903 | S | N—Ph | b2 | H | b8 | H |
| 2-3-904 | S | N—Ph | b3 | H | b8 | H |
| 2-3-905 | S | N—Ph | H | H | b9 | H |
| 2-3-906 | S | N—Ph | t-bu | H | b9 | H |
| 2-3-907 | S | N—Ph | methyl | H | b9 | H |
| 2-3-908 | S | N—Ph | b1 | H | b9 | H |
| 2-3-909 | S | N—Ph | b2 | H | b9 | H |
| 2-3-910 | S | N—Ph | b3 | H | b9 | H |
| 2-3-911 | S | N—Ph | H | H | b10 | H |
| 2-3-912 | S | N—Ph | t-bu | H | b10 | H |
| 2-3-913 | S | N—Ph | methyl | H | b10 | H |
| 2-3-914 | S | N—Ph | b1 | H | b10 | H |
| 2-3-915 | S | N—Ph | b2 | H | b10 | H |
| 2-3-916 | S | N—Ph | b3 | H | b10 | H |
| 2-3-917 | S | N—Ph | H | H | a1 | a1 |
| 2-3-918 | S | N—Ph | t-bu | H | a1 | a1 |
| 2-3-919 | S | N—Ph | methyl | H | a1 | a1 |
| 2-3-920 | S | N—Ph | b1 | H | a1 | a1 |
| 2-3-921 | S | N—Ph | b2 | H | a1 | a1 |
| 2-3-922 | S | N—Ph | b3 | H | a1 | a1 |
| 2-3-923 | S | N—Ph | H | H | a2 | a2 |
| 2-3-924 | S | N—Ph | t-bu | H | a2 | a2 |
| 2-3-925 | S | N—Ph | methyl | H | a2 | a2 |
| 2-3-926 | S | N—Ph | b1 | H | a2 | a2 |
| 2-3-927 | S | N—Ph | b2 | H | a2 | a2 |
| 2-3-928 | S | N—Ph | b3 | H | a2 | a2 |
| 2-3-929 | S | N—Ph | H | H | a3 | a3 |
| 2-3-930 | S | N—Ph | t-bu | H | a3 | a3 |
| 2-3-931 | S | N—Ph | methyl | H | a3 | a3 |
| 2-3-932 | S | N—Ph | b1 | H | a3 | a3 |
| 2-3-933 | S | N—Ph | b2 | H | a3 | a3 |
| 2-3-934 | S | N—Ph | b3 | H | a3 | a3 |
| 2-3-935 | S | N—Ph | H | H | b1 | b1 |
| 2-3-936 | S | N—Ph | t-bu | H | b1 | b1 |
| 2-3-937 | S | N—Ph | methyl | H | b1 | b1 |
| 2-3-938 | S | N—Ph | b1 | H | b1 | b1 |
| 2-3-939 | S | N—Ph | b2 | H | b1 | b1 |
| 2-3-940 | S | N—Ph | b3 | H | b1 | b1 |
| 2-3-941 | S | N—Ph | H | H | b2 | b2 |
| 2-3-942 | S | N—Ph | t-bu | H | b2 | b2 |
| 2-3-943 | S | N—Ph | methyl | H | b2 | b2 |
| 2-3-944 | S | N—Ph | b1 | H | b2 | b2 |
| 2-3-945 | S | N—Ph | b2 | H | b2 | b2 |
| 2-3-946 | S | N—Ph | b3 | H | b2 | b2 |
| 2-3-947 | S | N—Ph | H | H | b3 | b3 |
| 2-3-948 | S | N—Ph | t-bu | H | b3 | b3 |
| 2-3-949 | S | N—Ph | methyl | H | b3 | b3 |
| 2-3-950 | S | N—Ph | b1 | H | b3 | b3 |
| 2-3-951 | S | N—Ph | b2 | H | b3 | b3 |
| 2-3-952 | S | N—Ph | b3 | H | b3 | b3 |
| 2-3-953 | S | N—Ph | H | H | b5 | b5 |
| 2-3-954 | S | N—Ph | t-bu | H | b5 | b5 |
| 2-3-955 | S | N—Ph | methyl | H | b5 | b5 |
| 2-3-956 | S | N—Ph | b1 | H | b5 | b5 |
| 2-3-957 | S | N—Ph | b2 | H | b5 | b5 |
| 2-3-958 | S | N—Ph | b3 | H | b5 | b5 |
| 2-3-959 | S | N—Ph | H | H | b6 | b6 |
| 2-3-960 | S | N—Ph | t-bu | H | b6 | b6 |
| 2-3-961 | S | N—Ph | methyl | H | b6 | b6 |
| 2-3-962 | S | N—Ph | b1 | H | b6 | b6 |
| 2-3-963 | S | N—Ph | b2 | H | b6 | b6 |
| 2-3-964 | S | N—Ph | b3 | H | b6 | b6 |
| 2-3-965 | S | N—Ph | H | H | b7 | b7 |
| 2-3-966 | S | N—Ph | t-bu | H | b7 | b7 |
| 2-3-967 | S | N—Ph | methyl | H | b7 | b7 |
| 2-3-968 | S | N—Ph | b1 | H | b7 | b7 |
| 2-3-969 | S | N—Ph | b2 | H | b7 | b7 |
| 2-3-970 | S | N—Ph | b3 | H | b7 | b7 |

### Group 2-4,

The compounds are represented by the formula 2-4 below, wherein CY₁ and CY₃ are each a benzene group, X₁ is B, and Y1, Y2, R2a, R2c, (R1)a1, (R3)a3 are each shown in the table 4 below,

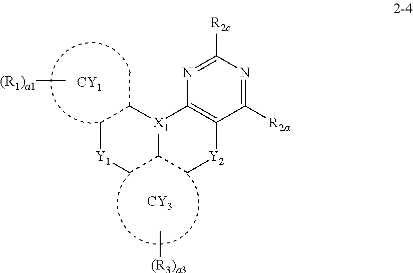

TABLE 4

| Compound No. | Y1 | Y2 | R2a | R2c | (R1)a1 | (R3)a3 |
|---|---|---|---|---|---|---|
| 2-4-001 | N—Ph | N—Ph | H | H | H | H |
| 2-4-002 | N—Ph | N—Ph | t-bu | t-bu | H | H |
| 2-4-003 | N—Ph | N—Ph | methyl | methyl | H | H |
| 2-4-004 | N—Ph | N—Ph | t-bu | t-bu | t-bu | t-bu |
| 2-4-005 | N—Ph | N—Ph | methyl | methyl | methyl | methyl |
| 2-4-006 | N—Ph | N—Ph | b1 | b1 | H | H |
| 2-4-007 | N—Ph | N—Ph | b2 | b2 | H | H |
| 2-4-008 | N—Ph | N—Ph | b3 | b3 | H | H |
| 2-4-009 | N—Ph | N—Ph | H | H | H | a1 |
| 2-4-010 | N—Ph | N—Ph | t-bu | t-bu | H | a1 |
| 2-4-011 | N—Ph | N—Ph | methyl | methyl | H | a1 |
| 2-4-012 | N—Ph | N—Ph | b1 | b1 | H | a1 |
| 2-4-013 | N—Ph | N—Ph | b2 | b2 | H | a1 |
| 2-4-014 | N—Ph | N—Ph | b3 | b3 | H | a1 |
| 2-4-015 | N—Ph | N—Ph | H | H | H | a2 |
| 2-4-016 | N—Ph | N—Ph | t-bu | t-bu | H | a2 |
| 2-4-017 | N—Ph | N—Ph | methyl | methyl | H | a2 |
| 2-4-018 | N—Ph | N—Ph | b1 | b1 | H | a2 |
| 2-4-019 | N—Ph | N—Ph | b2 | b2 | H | a2 |
| 2-4-020 | N—Ph | N—Ph | b3 | b3 | H | a2 |
| 2-4-021 | N—Ph | N—Ph | H | H | H | a3 |
| 2-4-022 | N—Ph | N—Ph | t-bu | t-bu | H | a3 |
| 2-4-023 | N—Ph | N—Ph | methyl | methyl | H | a3 |
| 2-4-024 | N—Ph | N—Ph | b1 | b1 | H | a3 |
| 2-4-025 | N—Ph | N—Ph | b2 | b2 | H | a3 |
| 2-4-026 | N—Ph | N—Ph | b3 | b3 | H | a3 |
| 2-4-027 | N—Ph | N—Ph | H | H | H | a4 |
| 2-4-028 | N—Ph | N—Ph | t-bu | t-bu | H | a4 |
| 2-4-029 | N—Ph | N—Ph | methyl | methyl | H | a4 |
| 2-4-030 | N—Ph | N—Ph | b1 | b1 | H | a4 |
| 2-4-031 | N—Ph | N—Ph | b2 | b2 | H | a4 |
| 2-4-032 | N—Ph | N—Ph | b3 | b3 | H | a4 |
| 2-4-033 | N—Ph | N—Ph | H | H | H | a5 |
| 2-4-034 | N—Ph | N—Ph | t-bu | t-bu | H | a5 |
| 2-4-035 | N—Ph | N—Ph | methyl | methyl | H | a5 |
| 2-4-036 | N—Ph | N—Ph | b1 | b1 | H | a5 |
| 2-4-037 | N—Ph | N—Ph | b2 | b2 | H | a5 |
| 2-4-038 | N—Ph | N—Ph | b3 | b3 | H | a5 |
| 2-4-039 | N—Ph | N—Ph | H | H | H | a6 |
| 2-4-040 | N—Ph | N—Ph | t-bu | t-bu | H | a6 |
| 2-4-041 | N—Ph | N—Ph | methyl | methyl | H | a6 |
| 2-4-042 | N—Ph | N—Ph | b1 | b1 | H | a6 |
| 2-4-043 | N—Ph | N—Ph | b2 | b2 | H | a6 |
| 2-4-044 | N—Ph | N—Ph | b3 | b3 | H | a6 |
| 2-4-045 | N—Ph | N—Ph | H | H | a1 | H |
| 2-4-046 | N—Ph | N—Ph | t-bu | t-bu | a1 | H |
| 2-4-047 | N—Ph | N—Ph | methyl | methyl | a1 | H |
| 2-4-048 | N—Ph | N—Ph | b1 | b1 | a1 | H |
| 2-4-049 | N—Ph | N—Ph | b2 | b2 | a1 | H |
| 2-4-050 | N—Ph | N—Ph | b3 | b3 | a1 | H |
| 2-4-051 | N—Ph | N—Ph | H | H | a2 | H |
| 2-4-052 | N—Ph | N—Ph | t-bu | t-bu | a2 | H |
| 2-4-053 | N—Ph | N—Ph | methyl | methyl | a2 | H |
| 2-4-054 | N—Ph | N—Ph | b1 | b1 | a2 | H |
| 2-4-055 | N—Ph | N—Ph | b2 | b2 | a2 | H |
| 2-4-056 | N—Ph | N—Ph | b3 | b3 | a2 | H |
| 2-4-057 | N—Ph | N—Ph | H | H | a3 | H |
| 2-4-058 | N—Ph | N—Ph | t-bu | t-bu | a3 | H |
| 2-4-059 | N—Ph | N—Ph | methyl | methyl | a3 | H |
| 2-4-060 | N—Ph | N—Ph | b1 | b1 | a3 | H |
| 2-4-061 | N—Ph | N—Ph | b2 | b2 | a3 | H |
| 2-4-062 | N—Ph | N—Ph | b3 | b3 | a3 | H |
| 2-4-063 | N—Ph | N—Ph | H | H | a4 | H |
| 2-4-064 | N—Ph | N—Ph | t-bu | t-bu | a4 | H |
| 2-4-065 | N—Ph | N—Ph | methyl | methyl | a4 | H |
| 2-4-066 | N—Ph | N—Ph | b1 | b1 | a4 | H |
| 2-4-067 | N—Ph | N—Ph | b2 | b2 | a4 | H |
| 2-4-068 | N—Ph | N—Ph | b3 | b3 | a4 | H |
| 2-4-069 | N—Ph | N—Ph | H | H | a5 | H |
| 2-4-070 | N—Ph | N—Ph | t-bu | t-bu | a5 | H |
| 2-4-071 | N—Ph | N—Ph | methyl | methyl | a5 | H |
| 2-4-072 | N—Ph | N—Ph | b1 | b1 | a5 | H |
| 2-4-073 | N—Ph | N—Ph | b2 | b2 | a5 | H |
| 2-4-074 | N—Ph | N—Ph | b3 | b3 | a5 | H |
| 2-4-075 | N—Ph | N—Ph | H | H | a6 | H |
| 2-4-076 | N—Ph | N—Ph | t-bu | t-bu | a6 | H |
| 2-4-077 | N—Ph | N—Ph | methyl | methyl | a6 | H |
| 2-4-078 | N—Ph | N—Ph | b1 | b1 | a6 | H |
| 2-4-079 | N—Ph | N—Ph | b2 | b2 | a6 | H |
| 2-4-080 | N—Ph | N—Ph | b3 | b3 | a6 | H |
| 2-4-081 | N—Ph | N—Ph | H | H | b1 | H |
| 2-4-082 | N—Ph | N—Ph | t-bu | t-bu | b1 | H |
| 2-4-083 | N—Ph | N—Ph | methyl | methyl | b1 | H |
| 2-4-084 | N—Ph | N—Ph | b1 | b1 | b1 | H |
| 2-4-085 | N—Ph | N—Ph | b2 | b2 | b1 | H |
| 2-4-086 | N—Ph | N—Ph | b3 | b3 | b1 | H |
| 2-4-087 | N—Ph | N—Ph | H | H | b2 | H |
| 2-4-088 | N—Ph | N—Ph | t-bu | t-bu | b2 | H |
| 2-4-089 | N—Ph | N—Ph | methyl | methyl | b2 | H |
| 2-4-090 | N—Ph | N—Ph | b1 | b1 | b2 | H |
| 2-4-091 | N—Ph | N—Ph | b2 | b2 | b2 | H |
| 2-4-092 | N—Ph | N—Ph | b3 | b3 | b2 | H |
| 2-4-093 | N—Ph | N—Ph | H | H | b3 | H |
| 2-4-094 | N—Ph | N—Ph | t-bu | t-bu | b3 | H |
| 2-4-095 | N—Ph | N—Ph | methyl | methyl | b3 | H |
| 2-4-096 | N—Ph | N—Ph | b1 | b1 | b3 | H |
| 2-4-097 | N—Ph | N—Ph | b2 | b2 | b3 | H |
| 2-4-098 | N—Ph | N—Ph | b3 | b3 | b3 | H |
| 2-4-099 | N—Ph | N—Ph | H | H | b4 | H |
| 2-4-100 | N—Ph | N—Ph | t-bu | t-bu | b4 | H |
| 2-4-101 | N—Ph | N—Ph | methyl | methyl | b4 | H |
| 2-4-102 | N—Ph | N—Ph | b1 | b1 | b4 | H |
| 2-4-103 | N—Ph | N—Ph | b2 | b2 | b4 | H |
| 2-4-104 | N—Ph | N—Ph | b3 | b3 | b4 | H |
| 2-4-105 | N—Ph | N—Ph | H | H | b5 | H |
| 2-4-106 | N—Ph | N—Ph | t-bu | t-bu | b5 | H |
| 2-4-107 | N—Ph | N—Ph | methyl | methyl | b5 | H |
| 2-4-108 | N—Ph | N—Ph | b1 | b1 | b5 | H |
| 2-4-109 | N—Ph | N—Ph | b2 | b2 | b5 | H |
| 2-4-110 | N—Ph | N—Ph | b3 | b3 | b5 | H |
| 2-4-111 | N—Ph | N—Ph | H | H | b6 | H |
| 2-4-112 | N—Ph | N—Ph | t-bu | t-bu | b6 | H |
| 2-4-113 | N—Ph | N—Ph | methyl | methyl | b6 | H |
| 2-4-114 | N—Ph | N—Ph | b1 | b1 | b6 | H |
| 2-4-115 | N—Ph | N—Ph | b2 | b2 | b6 | H |
| 2-4-116 | N—Ph | N—Ph | b3 | b3 | b6 | H |
| 2-4-117 | N—Ph | N—Ph | H | H | b7 | H |
| 2-4-118 | N—Ph | N—Ph | t-bu | t-bu | b7 | H |
| 2-4-119 | N—Ph | N—Ph | methyl | methyl | b7 | H |
| 2-4-120 | N—Ph | N—Ph | b1 | b1 | b7 | H |
| 2-4-121 | N—Ph | N—Ph | b2 | b2 | b7 | H |
| 2-4-122 | N—Ph | N—Ph | b3 | b3 | b7 | H |
| 2-4-123 | N—Ph | N—Ph | H | H | b8 | H |
| 2-4-124 | N—Ph | N—Ph | t-bu | t-bu | b8 | H |
| 2-4-125 | N—Ph | N—Ph | methyl | methyl | b8 | H |
| 2-4-126 | N—Ph | N—Ph | b1 | b1 | b8 | H |
| 2-4-127 | N—Ph | N—Ph | b2 | b2 | b8 | H |
| 2-4-128 | N—Ph | N—Ph | b3 | b3 | b8 | H |
| 2-4-129 | N—Ph | N—Ph | H | H | b9 | H |
| 2-4-130 | N—Ph | N—Ph | t-bu | t-bu | b9 | H |
| 2-4-131 | N—Ph | N—Ph | methyl | methyl | b9 | H |
| 2-4-132 | N—Ph | N—Ph | b1 | b1 | b9 | H |
| 2-4-133 | N—Ph | N—Ph | b2 | b2 | b9 | H |
| 2-4-134 | N—Ph | N—Ph | b3 | b3 | b9 | H |
| 2-4-135 | N—Ph | N—Ph | H | H | b10 | H |
| 2-4-136 | N—Ph | N—Ph | t-bu | t-bu | b10 | H |
| 2-4-137 | N—Ph | N—Ph | methyl | methyl | b10 | H |
| 2-4-138 | N—Ph | N—Ph | b1 | b1 | b10 | H |
| 2-4-139 | N—Ph | N—Ph | b2 | b2 | b10 | H |
| 2-4-140 | N—Ph | N—Ph | b3 | b3 | b10 | H |
| 2-4-141 | N—Ph | N—Ph | H | H | a1 | a1 |
| 2-4-142 | N—Ph | N—Ph | t-bu | t-bu | a1 | a1 |
| 2-4-143 | N—Ph | N—Ph | methyl | methyl | a1 | a1 |
| 2-4-144 | N—Ph | N—Ph | b1 | b1 | a1 | a1 |
| 2-4-145 | N—Ph | N—Ph | b2 | b2 | a1 | a1 |
| 2-4-146 | N—Ph | N—Ph | b3 | b3 | a1 | a1 |
| 2-4-147 | N—Ph | N—Ph | H | H | a2 | a2 |
| 2-4-148 | N—Ph | N—Ph | t-bu | t-bu | a2 | a2 |
| 2-4-149 | N—Ph | N—Ph | methyl | methyl | a2 | a2 |
| 2-4-150 | N—Ph | N—Ph | b1 | b1 | a2 | a2 |
| 2-4-151 | N—Ph | N—Ph | b2 | b2 | a2 | a2 |
| 2-4-152 | N—Ph | N—Ph | b3 | b3 | a2 | a2 |
| 2-4-153 | N—Ph | N—Ph | H | H | a3 | a3 |
| 2-4-154 | N—Ph | N—Ph | t-bu | t-bu | a3 | a3 |
| 2-4-155 | N—Ph | N—Ph | methyl | methyl | a3 | a3 |
| 2-4-156 | N—Ph | N—Ph | b1 | b1 | a3 | a3 |
| 2-4-157 | N—Ph | N—Ph | b2 | b2 | a3 | a3 |
| 2-4-158 | N—Ph | N—Ph | b3 | b3 | a3 | a3 |
| 2-4-159 | N—Ph | N—Ph | H | H | b1 | b1 |
| 2-4-160 | N—Ph | N—Ph | t-bu | t-bu | b1 | b1 |
| 2-4-161 | N—Ph | N—Ph | methyl | methyl | b1 | b1 |
| 2-4-162 | N—Ph | N—Ph | b1 | b1 | b1 | b1 |
| 2-4-163 | N—Ph | N—Ph | b2 | b2 | b1 | b1 |
| 2-4-164 | N—Ph | N—Ph | b3 | b3 | b1 | b1 |
| 2-4-165 | N—Ph | N—Ph | H | H | b2 | b2 |
| 2-4-166 | N—Ph | N—Ph | t-bu | t-bu | b2 | b2 |
| 2-4-167 | N—Ph | N—Ph | methyl | methyl | b2 | b2 |
| 2-4-168 | N—Ph | N—Ph | b1 | b1 | b2 | b2 |
| 2-4-169 | N—Ph | N—Ph | b2 | b2 | b2 | b2 |
| 2-4-170 | N—Ph | N—Ph | b3 | b3 | b2 | b2 |
| 2-4-171 | N—Ph | N—Ph | H | H | b3 | b3 |
| 2-4-172 | N—Ph | N—Ph | t-bu | t-bu | b3 | b3 |
| 2-4-173 | N—Ph | N—Ph | methyl | methyl | b3 | b3 |
| 2-4-174 | N—Ph | N—Ph | b1 | b1 | b3 | b3 |
| 2-4-175 | N—Ph | N—Ph | b2 | b2 | b3 | b3 |
| 2-4-176 | N—Ph | N—Ph | b3 | b3 | b3 | b3 |
| 2-4-177 | N—Ph | N—Ph | H | H | b5 | b5 |
| 2-4-178 | N—Ph | N—Ph | t-bu | t-bu | b5 | b5 |
| 2-4-179 | N—Ph | N—Ph | methyl | methyl | b5 | b5 |
| 2-4-180 | N—Ph | N—Ph | b1 | b1 | b5 | b5 |
| 2-4-181 | N—Ph | N—Ph | b2 | b2 | b5 | b5 |
| 2-4-182 | N—Ph | N—Ph | b3 | b3 | b5 | b5 |
| 2-4-183 | N—Ph | N—Ph | H | H | b6 | b6 |
| 2-4-184 | N—Ph | N—Ph | t-bu | t-bu | b6 | b6 |
| 2-4-185 | N—Ph | N—Ph | methyl | methyl | b6 | b6 |
| 2-4-186 | N—Ph | N—Ph | b1 | b1 | b6 | b6 |
| 2-4-187 | N—Ph | N—Ph | b2 | b2 | b6 | b6 |
| 2-4-188 | N—Ph | N—Ph | b3 | b3 | b6 | b6 |
| 2-4-189 | N—Ph | N—Ph | H | H | b7 | b7 |
| 2-4-190 | N—Ph | N—Ph | t-bu | t-bu | b7 | b7 |
| 2-4-191 | N—Ph | N—Ph | methyl | methyl | b7 | b7 |
| 2-4-192 | N—Ph | N—Ph | b1 | b1 | b7 | b7 |
| 2-4-193 | N—Ph | N—Ph | b2 | b2 | b7 | b7 |
| 2-4-194 | N—Ph | N—Ph | b3 | b3 | b7 | b7 |
| 2-4-195 | N-tolyl | N-tolyl | H | H | H | H |
| 2-4-196 | N-tolyl | N-tolyl | t-bu | t-bu | H | H |
| 2-4-197 | N-tolyl | N-tolyl | methyl | methyl | H | H |
| 2-4-198 | N-tolyl | N-tolyl | t-bu | t-bu | t-bu | t-bu |
| 2-4-199 | N-tolyl | N-tolyl | methyl | methyl | methyl | methyl |
| 2-4-200 | N-tolyl | N-tolyl | b1 | b1 | H | H |
| 2-4-201 | N-tolyl | N-tolyl | b2 | b2 | H | H |
| 2-4-202 | N-tolyl | N-tolyl | b3 | b3 | H | H |
| 2-4-203 | N-tolyl | N-tolyl | H | H | H | a1 |
| 2-4-204 | N-tolyl | N-tolyl | t-bu | t-bu | H | a1 |
| 2-4-205 | N-tolyl | N-tolyl | methyl | methyl | H | a1 |
| 2-4-206 | N-tolyl | N-tolyl | b1 | b1 | H | a1 |
| 2-4-207 | N-tolyl | N-tolyl | b2 | b2 | H | a1 |
| 2-4-208 | N-tolyl | N-tolyl | b3 | b3 | H | a1 |
| 2-4-209 | N-tolyl | N-tolyl | H | H | H | a2 |
| 2-4-210 | N-tolyl | N-tolyl | t-bu | t-bu | H | a2 |
| 2-4-211 | N-tolyl | N-tolyl | methyl | methyl | H | a2 |
| 2-4-212 | N-tolyl | N-tolyl | b1 | b1 | H | a2 |
| 2-4-213 | N-tolyl | N-tolyl | b2 | b2 | H | a2 |
| 2-4-214 | N-tolyl | N-tolyl | b3 | b3 | H | a2 |
| 2-4-215 | N-tolyl | N-tolyl | H | H | H | a3 |
| 2-4-216 | N-tolyl | N-tolyl | t-bu | t-bu | H | a3 |
| 2-4-217 | N-tolyl | N-tolyl | methyl | methyl | H | a3 |
| 2-4-218 | N-tolyl | N-tolyl | b1 | b1 | H | a3 |
| 2-4-219 | N-tolyl | N-tolyl | b2 | b2 | H | a3 |
| 2-4-220 | N-tolyl | N-tolyl | b3 | b3 | H | a3 |
| 2-4-221 | N-tolyl | N-tolyl | H | H | H | a4 |
| 2-4-222 | N-tolyl | N-tolyl | t-bu | t-bu | H | a4 |
| 2-4-223 | N-tolyl | N-tolyl | methyl | methyl | H | a4 |
| 2-4-224 | N-tolyl | N-tolyl | b1 | b1 | H | a4 |
| 2-4-225 | N-tolyl | N-tolyl | b2 | b2 | H | a4 |
| 2-4-226 | N-tolyl | N-tolyl | b3 | b3 | H | a4 |
| 2-4-227 | N-tolyl | N-tolyl | H | H | H | a5 |
| 2-4-228 | N-tolyl | N-tolyl | t-bu | t-bu | H | a5 |
| 2-4-229 | N-tolyl | N-tolyl | methyl | methyl | H | a5 |
| 2-4-230 | N-tolyl | N-tolyl | b1 | b1 | H | a5 |
| 2-4-231 | N-tolyl | N-tolyl | b2 | b2 | H | a5 |
| 2-4-232 | N-tolyl | N-tolyl | b3 | b3 | H | a5 |
| 2-4-233 | N-tolyl | N-tolyl | H | H | H | a6 |
| 2-4-234 | N-tolyl | N-tolyl | t-bu | t-bu | H | a6 |
| 2-4-235 | N-tolyl | N-tolyl | methyl | methyl | H | a6 |
| 2-4-236 | N-tolyl | N-tolyl | b1 | b1 | H | a6 |
| 2-4-237 | N-tolyl | N-tolyl | b2 | b2 | H | a6 |
| 2-4-238 | N-tolyl | N-tolyl | b3 | b3 | H | a6 |
| 2-4-239 | N-tolyl | N-tolyl | H | H | a1 | H |
| 2-4-240 | N-tolyl | N-tolyl | t-bu | t-bu | a1 | H |
| 2-4-241 | N-tolyl | N-tolyl | methyl | methyl | a1 | H |
| 2-4-242 | N-tolyl | N-tolyl | b1 | b1 | a1 | H |
| 2-4-243 | N-tolyl | N-tolyl | b2 | b2 | a1 | H |
| 2-4-244 | N-tolyl | N-tolyl | b3 | b3 | a1 | H |
| 2-4-245 | N-tolyl | N-tolyl | H | H | a2 | H |
| 2-4-246 | N-tolyl | N-tolyl | t-bu | t-bu | a2 | H |
| 2-4-247 | N-tolyl | N-tolyl | methyl | methyl | a2 | H |
| 2-4-248 | N-tolyl | N-tolyl | b1 | b1 | a2 | H |
| 2-4-249 | N-tolyl | N-tolyl | b2 | b2 | a2 | H |
| 2-4-250 | N-tolyl | N-tolyl | b3 | b3 | a2 | H |
| 2-4-251 | N-tolyl | N-tolyl | H | H | a3 | H |
| 2-4-252 | N-tolyl | N-tolyl | t-bu | t-bu | a3 | H |
| 2-4-253 | N-tolyl | N-tolyl | methyl | methyl | a3 | H |
| 2-4-254 | N-tolyl | N-tolyl | b1 | b1 | a3 | H |
| 2-4-255 | N-tolyl | N-tolyl | b2 | b2 | a3 | H |
| 2-4-256 | N-tolyl | N-tolyl | b3 | b3 | a3 | H |
| 2-4-257 | N-tolyl | N-tolyl | H | H | a4 | H |
| 2-4-258 | N-tolyl | N-tolyl | t-bu | t-bu | a4 | H |
| 2-4-259 | N-tolyl | N-tolyl | methyl | methyl | a4 | H |
| 2-4-260 | N-tolyl | N-tolyl | b1 | b1 | a4 | H |
| 2-4-261 | N-tolyl | N-tolyl | b2 | b2 | a4 | H |
| 2-4-262 | N-tolyl | N-tolyl | b3 | b3 | a4 | H |
| 2-4-263 | N-tolyl | N-tolyl | H | H | a5 | H |
| 2-4-264 | N-tolyl | N-tolyl | t-bu | t-bu | a5 | H |
| 2-4-265 | N-tolyl | N-tolyl | methyl | methyl | a5 | H |
| 2-4-266 | N-tolyl | N-tolyl | b1 | b1 | a5 | H |
| 2-4-267 | N-tolyl | N-tolyl | b2 | b2 | a5 | H |
| 2-4-268 | N-tolyl | N-tolyl | b3 | b3 | a5 | H |
| 2-4-269 | N-tolyl | N-tolyl | H | H | a6 | H |
| 2-4-270 | N-tolyl | N-tolyl | t-bu | t-bu | a6 | H |
| 2-4-271 | N-tolyl | N-tolyl | methyl | methyl | a6 | H |
| 2-4-272 | N-tolyl | N-tolyl | b1 | b1 | a6 | H |
| 2-4-273 | N-tolyl | N-tolyl | b2 | b2 | a6 | H |
| 2-4-274 | N-tolyl | N-tolyl | b3 | b3 | a6 | H |
| 2-4-275 | N-tolyl | N-tolyl | H | H | b1 | H |
| 2-4-276 | N-tolyl | N-tolyl | t-bu | t-bu | b1 | H |
| 2-4-277 | N-tolyl | N-tolyl | methyl | methyl | b1 | H |
| 2-4-278 | N-tolyl | N-tolyl | b1 | b1 | b1 | H |
| 2-4-279 | N-tolyl | N-tolyl | b2 | b2 | b1 | H |
| 2-4-280 | N-tolyl | N-tolyl | b3 | b3 | b1 | H |
| 2-4-281 | N-tolyl | N-tolyl | H | H | b2 | H |
| 2-4-282 | N-tolyl | N-tolyl | t-bu | t-bu | b2 | H |
| 2-4-283 | N-tolyl | N-tolyl | methyl | methyl | b2 | H |
| 2-4-284 | N-tolyl | N-tolyl | b1 | b1 | b2 | H |
| 2-4-285 | N-tolyl | N-tolyl | b2 | b2 | b2 | H |
| 2-4-286 | N-tolyl | N-tolyl | b3 | b3 | b2 | H |
| 2-4-287 | N-tolyl | N-tolyl | H | H | b3 | H |
| 2-4-288 | N-tolyl | N-tolyl | t-bu | t-bu | b3 | H |
| 2-4-289 | N-tolyl | N-tolyl | methyl | methyl | b3 | H |
| 2-4-290 | N-tolyl | N-tolyl | b1 | b1 | b3 | H |
| 2-4-291 | N-tolyl | N-tolyl | b2 | b2 | b3 | H |
| 2-4-292 | N-tolyl | N-tolyl | b3 | b3 | b3 | H |
| 2-4-293 | N-tolyl | N-tolyl | H | H | b4 | H |
| 2-4-294 | N-tolyl | N-tolyl | t-bu | t-bu | b4 | H |
| 2-4-295 | N-tolyl | N-tolyl | methyl | methyl | b4 | H |
| 2-4-296 | N-tolyl | N-tolyl | b1 | b1 | b4 | H |
| 2-4-297 | N-tolyl | N-tolyl | b2 | b2 | b4 | H |
| 2-4-298 | N-tolyl | N-tolyl | b3 | b3 | b4 | H |
| 2-4-299 | N-tolyl | N-tolyl | H | H | b5 | H |
| 2-4-300 | N-tolyl | N-tolyl | t-bu | t-bu | b5 | H |
| 2-4-301 | N-tolyl | N-tolyl | methyl | methyl | b5 | H |
| 2-4-302 | N-tolyl | N-tolyl | b1 | b1 | b5 | H |
| 2-4-303 | N-tolyl | N-tolyl | b2 | b2 | b5 | H |
| 2-4-304 | N-tolyl | N-tolyl | b3 | b3 | b5 | H |
| 2-4-305 | N-tolyl | N-tolyl | H | H | b6 | H |
| 2-4-306 | N-tolyl | N-tolyl | t-bu | t-bu | b6 | H |
| 2-4-307 | N-tolyl | N-tolyl | methyl | methyl | b6 | H |
| 2-4-308 | N-tolyl | N-tolyl | b1 | b1 | b6 | H |
| 2-4-309 | N-tolyl | N-tolyl | b2 | b2 | b6 | H |
| 2-4-310 | N-tolyl | N-tolyl | b3 | b3 | b6 | H |
| 2-4-311 | N-tolyl | N-tolyl | H | H | b7 | H |
| 2-4-312 | N-tolyl | N-tolyl | t-bu | t-bu | b7 | H |
| 2-4-313 | N-tolyl | N-tolyl | methyl | methyl | b7 | H |
| 2-4-314 | N-tolyl | N-tolyl | b1 | b1 | b7 | H |
| 2-4-315 | N-tolyl | N-tolyl | b2 | b2 | b7 | H |
| 2-4-316 | N-tolyl | N-tolyl | b3 | b3 | b7 | H |
| 2-4-317 | N-tolyl | N-tolyl | H | H | b8 | H |
| 2-4-318 | N-tolyl | N-tolyl | t-bu | t-bu | b8 | H |
| 2-4-319 | N-tolyl | N-tolyl | methyl | methyl | b8 | H |
| 2-4-320 | N-tolyl | N-tolyl | b1 | b1 | b8 | H |
| 2-4-321 | N-tolyl | N-tolyl | b2 | b2 | b8 | H |
| 2-4-322 | N-tolyl | N-tolyl | b3 | b3 | b8 | H |
| 2-4-323 | N-tolyl | N-tolyl | H | H | b9 | H |
| 2-4-324 | N-tolyl | N-tolyl | t-bu | t-bu | b9 | H |
| 2-4-325 | N-tolyl | N-tolyl | methyl | methyl | b9 | H |
| 2-4-326 | N-tolyl | N-tolyl | b1 | b1 | b9 | H |
| 2-4-327 | N-tolyl | N-tolyl | b2 | b2 | b9 | H |
| 2-4-328 | N-tolyl | N-tolyl | b3 | b3 | b9 | H |
| 2-4-329 | N-tolyl | N-tolyl | H | H | b10 | H |
| 2-4-330 | N-tolyl | N-tolyl | t-bu | t-bu | b10 | H |
| 2-4-331 | N-tolyl | N-tolyl | methyl | methyl | b10 | H |
| 2-4-332 | N-tolyl | N-tolyl | b1 | b1 | b10 | H |
| 2-4-333 | N-tolyl | N-tolyl | b2 | b2 | b10 | H |
| 2-4-334 | N-tolyl | N-tolyl | b3 | b3 | b10 | H |
| 2-4-335 | N-tolyl | N-tolyl | H | H | a1 | a1 |
| 2-4-336 | N-tolyl | N-tolyl | t-bu | t-bu | a1 | a1 |
| 2-4-337 | N-tolyl | N-tolyl | methyl | methyl | a1 | a1 |
| 2-4-338 | N-tolyl | N-tolyl | b1 | b1 | a1 | a1 |
| 2-4-339 | N-tolyl | N-tolyl | b2 | b2 | a1 | a1 |
| 2-4-340 | N-tolyl | N-tolyl | b3 | b3 | a1 | a1 |
| 2-4-341 | N-tolyl | N-tolyl | H | H | a2 | a2 |
| 2-4-342 | N-tolyl | N-tolyl | t-bu | t-bu | a2 | a2 |
| 2-4-343 | N-tolyl | N-tolyl | methyl | methyl | a2 | a2 |
| 2-4-344 | N-tolyl | N-tolyl | b1 | b1 | a2 | a2 |
| 2-4-345 | N-tolyl | N-tolyl | b2 | b2 | a2 | a2 |
| 2-4-346 | N-tolyl | N-tolyl | b3 | b3 | a2 | a2 |
| 2-4-347 | N-tolyl | N-tolyl | H | H | a3 | a3 |
| 2-4-348 | N-tolyl | N-tolyl | t-bu | t-bu | a3 | a3 |
| 2-4-349 | N-tolyl | N-tolyl | methyl | methyl | a3 | a3 |
| 2-4-350 | N-tolyl | N-tolyl | b1 | b1 | a3 | a3 |
| 2-4-351 | N-tolyl | N-tolyl | b2 | b2 | a3 | a3 |
| 2-4-352 | N-tolyl | N-tolyl | b3 | b3 | a3 | a3 |
| 2-4-353 | N-tolyl | N-tolyl | H | H | b1 | b1 |
| 2-4-354 | N-tolyl | N-tolyl | t-bu | t-bu | b1 | b1 |
| 2-4-355 | N-tolyl | N-tolyl | methyl | methyl | b1 | b1 |
| 2-4-356 | N-tolyl | N-tolyl | b1 | b1 | b1 | b1 |
| 2-4-357 | N-tolyl | N-tolyl | b2 | b2 | b1 | b1 |
| 2-4-358 | N-tolyl | N-tolyl | b3 | b3 | b1 | b1 |
| 2-4-359 | N-tolyl | N-tolyl | H | H | b2 | b2 |
| 2-4-360 | N-tolyl | N-tolyl | t-bu | t-bu | b2 | b2 |
| 2-4-361 | N-tolyl | N-tolyl | methyl | methyl | b2 | b2 |
| 2-4-362 | N-tolyl | N-tolyl | b1 | b1 | b2 | b2 |
| 2-4-363 | N-tolyl | N-tolyl | b2 | b2 | b2 | b2 |
| 2-4-364 | N-tolyl | N-tolyl | b3 | b3 | b2 | b2 |
| 2-4-365 | N-tolyl | N-tolyl | H | H | b3 | b3 |
| 2-4-366 | N-tolyl | N-tolyl | t-bu | t-bu | b3 | b3 |
| 2-4-367 | N-tolyl | N-tolyl | methyl | methyl | b3 | b3 |
| 2-4-368 | N-tolyl | N-tolyl | b1 | b1 | b3 | b3 |
| 2-4-369 | N-tolyl | N-tolyl | b2 | b2 | b3 | b3 |
| 2-4-370 | N-tolyl | N-tolyl | b3 | b3 | b3 | b3 |
| 2-4-371 | N-tolyl | N-tolyl | H | H | b5 | b5 |
| 2-4-372 | N-tolyl | N-tolyl | t-bu | t-bu | b5 | b5 |
| 2-4-373 | N-tolyl | N-tolyl | methyl | methyl | b5 | b5 |
| 2-4-374 | N-tolyl | N-tolyl | b1 | b1 | b5 | b5 |
| 2-4-375 | N-tolyl | N-tolyl | b2 | b2 | b5 | b5 |
| 2-4-376 | N-tolyl | N-tolyl | b3 | b3 | b5 | b5 |
| 2-4-377 | N-tolyl | N-tolyl | H | H | b6 | b6 |
| 2-4-378 | N-tolyl | N-tolyl | t-bu | t-bu | b6 | b6 |
| 2-4-379 | N-tolyl | N-tolyl | methyl | methyl | b6 | b6 |
| 2-4-380 | N-tolyl | N-tolyl | b1 | b1 | b6 | b6 |
| 2-4-381 | N-tolyl | N-tolyl | b2 | b2 | b6 | b6 |
| 2-4-382 | N-tolyl | N-tolyl | b3 | b3 | b6 | b6 |
| 2-4-383 | N-tolyl | N-tolyl | H | H | b7 | b7 |
| 2-4-384 | N-tolyl | N-tolyl | t-bu | t-bu | b7 | b7 |
| 2-4-385 | N-tolyl | N-tolyl | methyl | methyl | b7 | b7 |
| 2-4-386 | N-tolyl | N-tolyl | b1 | b1 | b7 | b7 |
| 2-4-387 | N-tolyl | N-tolyl | b2 | b2 | b7 | b7 |
| 2-4-388 | N-tolyl | N-tolyl | b3 | b3 | b7 | b7 |
| 2-4-389 | N-Biph | N-Biph | H | H | H | H |
| 2-4-390 | N-Biph | N-Biph | t-bu | t-bu | H | H |
| 2-4-391 | N-Biph | N-Biph | methyl | methyl | H | H |
| 2-4-392 | N-Biph | N-Biph | t-bu | t-bu | t-bu | t-bu |
| 2-4-393 | N-Biph | N-Biph | methyl | methyl | methyl | methyl |
| 2-4-394 | N-Biph | N-Biph | b1 | b1 | H | H |
| 2-4-395 | N-Biph | N-Biph | b2 | b2 | H | H |
| 2-4-396 | N-Biph | N-Biph | b3 | b3 | H | H |
| 2-4-397 | N-Biph | N-Biph | H | H | H | a1 |
| 2-4-398 | N-Biph | N-Biph | t-bu | t-bu | H | a1 |
| 2-4-399 | N-Biph | N-Biph | methyl | methyl | H | a1 |
| 2-4-400 | N-Biph | N-Biph | b1 | b1 | H | a1 |
| 2-4-401 | N-Biph | N-Biph | b2 | b2 | H | a1 |
| 2-4-402 | N-Biph | N-Biph | b3 | b3 | H | a1 |
| 2-4-403 | N-Biph | N-Biph | H | H | H | a2 |
| 2-4-404 | N-Biph | N-Biph | t-bu | t-bu | H | a2 |
| 2-4-405 | N-Biph | N-Biph | methyl | methyl | H | a2 |
| 2-4-406 | N-Biph | N-Biph | b1 | b1 | H | a2 |
| 2-4-407 | N-Biph | N-Biph | b2 | b2 | H | a2 |
| 2-4-408 | N-Biph | N-Biph | b3 | b3 | H | a2 |
| 2-4-409 | N-Biph | N-Biph | H | H | H | a3 |
| 2-4-410 | N-Biph | N-Biph | t-bu | t-bu | H | a3 |
| 2-4-411 | N-Biph | N-Biph | methyl | methyl | H | a3 |
| 2-4-412 | N-Biph | N-Biph | b1 | b1 | H | a3 |
| 2-4-413 | N-Biph | N-Biph | b2 | b2 | H | a3 |
| 2-4-414 | N-Biph | N-Biph | b3 | b3 | H | a3 |
| 2-4-415 | N-Biph | N-Biph | H | H | H | a4 |
| 2-4-416 | N-Biph | N-Biph | t-bu | t-bu | H | a4 |
| 2-4-417 | N-Biph | N-Biph | methyl | methyl | H | a4 |
| 2-4-418 | N-Biph | N-Biph | b1 | b1 | H | a4 |
| 2-4-419 | N-Biph | N-Biph | b2 | b2 | H | a4 |
| 2-4-420 | N-Biph | N-Biph | b3 | b3 | H | a4 |
| 2-4-421 | N-Biph | N-Biph | H | H | H | a5 |
| 2-4-422 | N-Biph | N-Biph | t-bu | t-bu | H | a5 |
| 2-4-423 | N-Biph | N-Biph | methyl | methyl | H | a5 |
| 2-4-424 | N-Biph | N-Biph | b1 | b1 | H | a5 |
| 2-4-425 | N-Biph | N-Biph | b2 | b2 | H | a5 |
| 2-4-426 | N-Biph | N-Biph | b3 | b3 | H | a5 |
| 2-4-427 | N-Biph | N-Biph | H | H | H | a6 |
| 2-4-428 | N-Biph | N-Biph | t-bu | t-bu | H | a6 |
| 2-4-429 | N-Biph | N-Biph | methyl | methyl | H | a6 |
| 2-4-430 | N-Biph | N-Biph | b1 | b1 | H | a6 |
| 2-4-431 | N-Biph | N-Biph | b2 | b2 | H | a6 |
| 2-4-432 | N-Biph | N-Biph | b3 | b3 | H | a6 |
| 2-4-433 | N-Biph | N-Biph | H | H | a1 | H |
| 2-4-434 | N-Biph | N-Biph | t-bu | t-bu | a1 | H |
| 2-4-435 | N-Biph | N-Biph | methyl | methyl | a1 | H |
| 2-4-436 | N-Biph | N-Biph | b1 | b1 | a1 | H |
| 2-4-437 | N-Biph | N-Biph | b2 | b2 | a1 | H |
| 2-4-438 | N-Biph | N-Biph | b3 | b3 | a1 | H |
| 2-4-439 | N-Biph | N-Biph | H | H | a2 | H |
| 2-4-440 | N-Biph | N-Biph | t-bu | t-bu | a2 | H |
| 2-4-441 | N-Biph | N-Biph | methyl | methyl | a2 | H |
| 2-4-442 | N-Biph | N-Biph | b1 | b1 | a2 | H |
| 2-4-443 | N-Biph | N-Biph | b2 | b2 | a2 | H |
| 2-4-444 | N-Biph | N-Biph | b3 | b3 | a2 | H |
| 2-4-445 | N-Biph | N-Biph | H | H | a3 | H |
| 2-4-446 | N-Biph | N-Biph | t-bu | t-bu | a3 | H |
| 2-4-447 | N-Biph | N-Biph | methyl | methyl | a3 | H |
| 2-4-448 | N-Biph | N-Biph | b1 | b1 | a3 | H |
| 2-4-449 | N-Biph | N-Biph | b2 | b2 | a3 | H |
| 2-4-450 | N-Biph | N-Biph | b3 | b3 | a3 | H |
| 2-4-451 | N-Biph | N-Biph | H | H | a4 | H |
| 2-4-452 | N-Biph | N-Biph | t-bu | t-bu | a4 | H |
| 2-4-453 | N-Biph | N-Biph | methyl | methyl | a4 | H |
| 2-4-454 | N-Biph | N-Biph | b1 | b1 | a4 | H |
| 2-4-455 | N-Biph | N-Biph | b2 | b2 | a4 | H |
| 2-4-456 | N-Biph | N-Biph | b3 | b3 | a4 | H |
| 2-4-457 | N-Biph | N-Biph | H | H | a5 | H |
| 2-4-458 | N-Biph | N-Biph | t-bu | t-bu | a5 | H |
| 2-4-459 | N-Biph | N-Biph | methyl | methyl | a5 | H |
| 2-4-460 | N-Biph | N-Biph | b1 | b1 | a5 | H |
| 2-4-461 | N-Biph | N-Biph | b2 | b2 | a5 | H |
| 2-4-462 | N-Biph | N-Biph | b3 | b3 | a5 | H |
| 2-4-463 | N-Biph | N-Biph | H | H | a6 | H |
| 2-4-464 | N-Biph | N-Biph | t-bu | t-bu | a6 | H |
| 2-4-465 | N-Biph | N-Biph | methyl | methyl | a6 | H |
| 2-4-466 | N-Biph | N-Biph | b1 | b1 | a6 | H |
| 2-4-467 | N-Biph | N-Biph | b2 | b2 | a6 | H |
| 2-4-468 | N-Biph | N-Biph | b3 | b3 | a6 | H |
| 2-4-469 | N-Biph | N-Biph | H | H | b1 | H |
| 2-4-470 | N-Biph | N-Biph | t-bu | t-bu | b1 | H |
| 2-4-471 | N-Biph | N-Biph | methyl | methyl | b1 | H |
| 2-4-472 | N-Biph | N-Biph | b1 | b1 | b1 | H |
| 2-4-473 | N-Biph | N-Biph | b2 | b2 | b1 | H |
| 2-4-474 | N-Biph | N-Biph | b3 | b3 | b1 | H |
| 2-4-475 | N-Biph | N-Biph | H | H | b2 | H |
| 2-4-476 | N-Biph | N-Biph | t-bu | t-bu | b2 | H |
| 2-4-477 | N-Biph | N-Biph | methyl | methyl | b2 | H |
| 2-4-478 | N-Biph | N-Biph | b1 | b1 | b2 | H |
| 2-4-479 | N-Biph | N-Biph | b2 | b2 | b2 | H |
| 2-4-480 | N-Biph | N-Biph | b3 | b3 | b2 | H |
| 2-4-481 | N-Biph | N-Biph | H | H | b3 | H |
| 2-4-482 | N-Biph | N-Biph | t-bu | t-bu | b3 | H |
| 2-4-483 | N-Biph | N-Biph | methyl | methyl | b3 | H |
| 2-4-484 | N-Biph | N-Biph | b1 | b1 | b3 | H |
| 2-4-485 | N-Biph | N-Biph | b2 | b2 | b3 | H |
| 2-4-486 | N-Biph | N-Biph | b3 | b3 | b3 | H |
| 2-4-487 | N-Biph | N-Biph | H | H | b4 | H |
| 2-4-488 | N-Biph | N-Biph | t-bu | t-bu | b4 | H |
| 2-4-489 | N-Biph | N-Biph | methyl | methyl | b4 | H |
| 2-4-490 | N-Biph | N-Biph | b1 | b1 | b4 | H |
| 2-4-491 | N-Biph | N-Biph | b2 | b2 | b4 | H |
| 2-4-492 | N-Biph | N-Biph | b3 | b3 | b4 | H |
| 2-4-493 | N-Biph | N-Biph | H | H | b5 | H |
| 2-4-494 | N-Biph | N-Biph | t-bu | t-bu | b5 | H |
| 2-4-495 | N-Biph | N-Biph | methyl | methyl | b5 | H |
| 2-4-496 | N-Biph | N-Biph | b1 | b1 | b5 | H |
| 2-4-497 | N-Biph | N-Biph | b2 | b2 | b5 | H |
| 2-4-498 | N-Biph | N-Biph | b3 | b3 | b5 | H |
| 2-4-499 | N-Biph | N-Biph | H | H | b6 | H |
| 2-4-500 | N-Biph | N-Biph | t-bu | t-bu | b6 | H |
| 2-4-501 | N-Biph | N-Biph | methyl | methyl | b6 | H |
| 2-4-502 | N-Biph | N-Biph | b1 | b1 | b6 | H |
| 2-4-503 | N-Biph | N-Biph | b2 | b2 | b6 | H |
| 2-4-504 | N-Biph | N-Biph | b3 | b3 | b6 | H |
| 2-4-505 | N-Biph | N-Biph | H | H | b7 | H |
| 2-4-506 | N-Biph | N-Biph | t-bu | t-bu | b7 | H |
| 2-4-507 | N-Biph | N-Biph | methyl | methyl | b7 | H |
| 2-4-508 | N-Biph | N-Biph | b1 | b1 | b7 | H |
| 2-4-509 | N-Biph | N-Biph | b2 | b2 | b7 | H |
| 2-4-510 | N-Biph | N-Biph | b3 | b3 | b7 | H |
| 2-4-511 | N-Biph | N-Biph | H | H | b8 | H |
| 2-4-512 | N-Biph | N-Biph | t-bu | t-bu | b8 | H |
| 2-4-513 | N-Biph | N-Biph | methyl | methyl | b8 | H |
| 2-4-514 | N-Biph | N-Biph | b1 | b1 | b8 | H |
| 2-4-515 | N-Biph | N-Biph | b2 | b2 | b8 | H |
| 2-4-516 | N-Biph | N-Biph | b3 | b3 | b8 | H |
| 2-4-517 | N-Biph | N-Biph | H | H | b9 | H |
| 2-4-518 | N-Biph | N-Biph | t-bu | t-bu | b9 | H |
| 2-4-519 | N-Biph | N-Biph | methyl | methyl | b9 | H |
| 2-4-520 | N-Biph | N-Biph | b1 | b1 | b9 | H |
| 2-4-521 | N-Biph | N-Biph | b2 | b2 | b9 | H |
| 2-4-522 | N-Biph | N-Biph | b3 | b3 | b9 | H |
| 2-4-523 | N-Biph | N-Biph | H | H | b10 | H |
| 2-4-524 | N-Biph | N-Biph | t-bu | t-bu | b10 | H |
| 2-4-525 | N-Biph | N-Biph | methyl | methyl | b10 | H |
| 2-4-526 | N-Biph | N-Biph | b1 | b1 | b10 | H |
| 2-4-527 | N-Biph | N-Biph | b2 | b2 | b10 | H |
| 2-4-528 | N-Biph | N-Biph | b3 | b3 | b10 | H |
| 2-4-529 | N-Biph | N-Biph | H | H | a1 | a1 |
| 2-4-530 | N-Biph | N-Biph | t-bu | t-bu | a1 | a1 |
| 2-4-531 | N-Biph | N-Biph | methyl | methyl | a1 | a1 |
| 2-4-532 | N-Biph | N-Biph | b1 | b1 | a1 | a1 |
| 2-4-533 | N-Biph | N-Biph | b2 | b2 | a1 | a1 |
| 2-4-534 | N-Biph | N-Biph | b3 | b3 | a1 | a1 |
| 2-4-535 | N-Biph | N-Biph | H | H | a2 | a2 |
| 2-4-536 | N-Biph | N-Biph | t-bu | t-bu | a2 | a2 |
| 2-4-537 | N-Biph | N-Biph | methyl | methyl | a2 | a2 |
| 2-4-538 | N-Biph | N-Biph | b1 | b1 | a2 | a2 |
| 2-4-539 | N-Biph | N-Biph | b2 | b2 | a2 | a2 |
| 2-4-540 | N-Biph | N-Biph | b3 | b3 | a2 | a2 |
| 2-4-541 | N-Biph | N-Biph | H | H | a3 | a3 |
| 2-4-542 | N-Biph | N-Biph | t-bu | t-bu | a3 | a3 |
| 2-4-543 | N-Biph | N-Biph | methyl | methyl | a3 | a3 |
| 2-4-544 | N-Biph | N-Biph | b1 | b1 | a3 | a3 |
| 2-4-545 | N-Biph | N-Biph | b2 | b2 | a3 | a3 |
| 2-4-546 | N-Biph | N-Biph | b3 | b3 | a3 | a3 |
| 2-4-547 | N-Biph | N-Biph | H | H | b1 | b1 |
| 2-4-548 | N-Biph | N-Biph | t-bu | t-bu | b1 | b1 |
| 2-4-549 | N-Biph | N-Biph | methyl | methyl | b1 | b1 |
| 2-4-550 | N-Biph | N-Biph | b1 | b1 | b1 | b1 |
| 2-4-551 | N-Biph | N-Biph | b2 | b2 | b1 | b1 |
| 2-4-552 | N-Biph | N-Biph | b3 | b3 | b1 | b1 |
| 2-4-553 | N-Biph | N-Biph | H | H | b2 | b2 |
| 2-4-554 | N-Biph | N-Biph | t-bu | t-bu | b2 | b2 |
| 2-4-555 | N-Biph | N-Biph | methyl | methyl | b2 | b2 |
| 2-4-556 | N-Biph | N-Biph | b1 | b1 | b2 | b2 |
| 2-4-557 | N-Biph | N-Biph | b2 | b2 | b2 | b2 |
| 2-4-558 | N-Biph | N-Biph | b3 | b3 | b2 | b2 |
| 2-4-559 | N-Biph | N-Biph | H | H | b3 | b3 |
| 2-4-560 | N-Biph | N-Biph | t-bu | t-bu | b3 | b3 |
| 2-4-561 | N-Biph | N-Biph | methyl | methyl | b3 | b3 |
| 2-4-562 | N-Biph | N-Biph | b1 | b1 | b3 | b3 |
| 2-4-563 | N-Biph | N-Biph | b2 | b2 | b3 | b3 |
| 2-4-564 | N-Biph | N-Biph | b3 | b3 | b3 | b3 |
| 2-4-565 | N-Biph | N-Biph | H | H | b5 | b5 |
| 2-4-566 | N-Biph | N-Biph | t-bu | t-bu | b5 | b5 |
| 2-4-567 | N-Biph | N-Biph | methyl | methyl | b5 | b5 |
| 2-4-568 | N-Biph | N-Biph | b1 | b1 | b5 | b5 |
| 2-4-569 | N-Biph | N-Biph | b2 | b2 | b5 | b5 |
| 2-4-570 | N-Biph | N-Biph | b3 | b3 | b5 | b5 |
| 2-4-571 | N-Biph | N-Biph | H | H | b6 | b6 |
| 2-4-572 | N-Biph | N-Biph | t-bu | t-bu | b6 | b6 |
| 2-4-573 | N-Biph | N-Biph | methyl | methyl | b6 | b6 |
| 2-4-574 | N-Biph | N-Biph | b1 | b1 | b6 | b6 |
| 2-4-575 | N-Biph | N-Biph | b2 | b2 | b6 | b6 |
| 2-4-576 | N-Biph | N-Biph | b3 | b3 | b6 | b6 |
| 2-4-577 | N-Biph | N-Biph | H | H | b7 | b7 |
| 2-4-578 | N-Biph | N-Biph | t-bu | t-bu | b7 | b7 |
| 2-4-579 | N-Biph | N-Biph | methyl | methyl | b7 | b7 |
| 2-4-580 | N-Biph | N-Biph | b1 | b1 | b7 | b7 |
| 2-4-581 | N-Biph | N-Biph | b2 | b2 | b7 | b7 |
| 2-4-582 | N-Biph | N-Biph | b3 | b3 | b7 | b7 |
| 2-4-583 | O | N—Ph | H | H | H | H |
| 2-4-584 | O | N—Ph | t-bu | t-bu | H | H |
| 2-4-585 | O | N—Ph | methyl | methyl | H | H |
| 2-4-586 | O | N—Ph | t-bu | t-bu | t-bu | t-bu |
| 2-4-587 | O | N—Ph | methyl | methyl | methyl | methyl |
| 2-4-588 | O | N—Ph | b1 | b1 | H | H |
| 2-4-589 | O | N—Ph | b2 | b2 | H | H |
| 2-4-590 | O | N—Ph | b3 | b3 | H | H |
| 2-4-591 | O | N—Ph | H | H | H | a1 |
| 2-4-592 | O | N—Ph | t-bu | t-bu | H | a1 |
| 2-4-593 | O | N—Ph | methyl | methyl | H | a1 |
| 2-4-594 | O | N—Ph | b1 | b1 | H | a1 |
| 2-4-595 | O | N—Ph | b2 | b2 | H | a1 |
| 2-4-596 | O | N—Ph | b3 | b3 | H | a1 |
| 2-4-597 | O | N—Ph | H | H | H | a2 |
| 2-4-598 | O | N—Ph | t-bu | t-bu | H | a2 |
| 2-4-599 | O | N—Ph | methyl | methyl | H | a2 |
| 2-4-600 | O | N—Ph | b1 | b1 | H | a2 |
| 2-4-601 | O | N—Ph | b2 | b2 | H | a2 |
| 2-4-602 | O | N—Ph | b3 | b3 | H | a2 |
| 2-4-603 | O | N—Ph | H | H | H | a3 |
| 2-4-604 | O | N—Ph | t-bu | t-bu | H | a3 |
| 2-4-605 | O | N—Ph | methyl | methyl | H | a3 |
| 2-4-606 | O | N—Ph | b1 | b1 | H | a3 |
| 2-4-607 | O | N—Ph | b2 | b2 | H | a3 |
| 2-4-608 | O | N—Ph | b3 | b3 | H | a3 |
| 2-4-609 | O | N—Ph | H | H | H | a4 |
| 2-4-610 | O | N—Ph | t-bu | t-bu | H | a4 |
| 2-4-611 | O | N—Ph | methyl | methyl | H | a4 |
| 2-4-612 | O | N—Ph | b1 | b1 | H | a4 |
| 2-4-613 | O | N—Ph | b2 | b2 | H | a4 |
| 2-4-614 | O | N—Ph | b3 | b3 | H | a4 |
| 2-4-615 | O | N—Ph | H | H | H | a5 |
| 2-4-616 | O | N—Ph | t-bu | t-bu | H | a5 |
| 2-4-617 | O | N—Ph | methyl | methyl | H | a5 |
| 2-4-618 | O | N—Ph | b1 | b1 | H | a5 |
| 2-4-619 | O | N—Ph | b2 | b2 | H | a5 |
| 2-4-620 | O | N—Ph | b3 | b3 | H | a5 |
| 2-4-621 | O | N—Ph | H | H | H | a6 |
| 2-4-622 | O | N—Ph | t-bu | t-bu | H | a6 |
| 2-4-623 | O | N—Ph | methyl | methyl | H | a6 |
| 2-4-624 | O | N—Ph | b1 | b1 | H | a6 |
| 2-4-625 | O | N—Ph | b2 | b2 | H | a6 |
| 2-4-626 | O | N—Ph | b3 | b3 | H | a6 |
| 2-4-627 | O | N—Ph | H | H | a1 | H |
| 2-4-628 | O | N—Ph | t-bu | t-bu | a1 | H |
| 2-4-629 | O | N—Ph | methyl | methyl | a1 | H |
| 2-4-630 | O | N—Ph | b1 | b1 | a1 | H |
| 2-4-631 | O | N—Ph | b2 | b2 | a1 | H |
| 2-4-632 | O | N—Ph | b3 | b3 | a1 | H |
| 2-4-633 | O | N—Ph | H | H | a2 | H |
| 2-4-634 | O | N—Ph | t-bu | t-bu | a2 | H |
| 2-4-635 | O | N—Ph | methyl | methyl | a2 | H |
| 2-4-636 | O | N—Ph | b1 | b1 | a2 | H |
| 2-4-637 | O | N—Ph | b2 | b2 | a2 | H |
| 2-4-638 | O | N—Ph | b3 | b3 | a2 | H |
| 2-4-639 | O | N—Ph | H | H | a3 | H |
| 2-4-640 | O | N—Ph | t-bu | t-bu | a3 | H |
| 2-4-641 | O | N—Ph | methyl | methyl | a3 | H |
| 2-4-642 | O | N—Ph | b1 | b1 | a3 | H |
| 2-4-643 | O | N—Ph | b2 | b2 | a3 | H |
| 2-4-644 | O | N—Ph | b3 | b3 | a3 | H |
| 2-4-645 | O | N—Ph | H | H | a4 | H |
| 2-4-646 | O | N—Ph | t-bu | t-bu | a4 | H |
| 2-4-647 | O | N—Ph | methyl | methyl | a4 | H |
| 2-4-648 | O | N—Ph | b1 | b1 | a4 | H |
| 2-4-649 | O | N—Ph | b2 | b2 | a4 | H |
| 2-4-650 | O | N—Ph | b3 | b3 | a4 | H |
| 2-4-651 | O | N—Ph | H | H | a5 | H |
| 2-4-652 | O | N—Ph | t-bu | t-bu | a5 | H |
| 2-4-653 | O | N—Ph | methyl | methyl | a5 | H |
| 2-4-654 | O | N—Ph | b1 | b1 | a5 | H |
| 2-4-655 | O | N—Ph | b2 | b2 | a5 | H |
| 2-4-656 | O | N—Ph | b3 | b3 | a5 | H |
| 2-4-657 | O | N—Ph | H | H | a6 | H |
| 2-4-658 | O | N—Ph | t-bu | t-bu | a6 | H |
| 2-4-659 | O | N—Ph | methyl | methyl | a6 | H |
| 2-4-660 | O | N—Ph | b1 | b1 | a6 | H |
| 2-4-661 | O | N—Ph | b2 | b2 | a6 | H |
| 2-4-662 | O | N—Ph | b3 | b3 | a6 | H |
| 2-4-663 | O | N—Ph | H | H | b1 | H |
| 2-4-664 | O | N—Ph | t-bu | t-bu | b1 | H |
| 2-4-665 | O | N—Ph | methyl | methyl | b1 | H |
| 2-4-666 | O | N—Ph | b1 | b1 | b1 | H |
| 2-4-667 | O | N—Ph | b2 | b2 | b1 | H |
| 2-4-668 | O | N—Ph | b3 | b3 | b1 | H |
| 2-4-669 | O | N—Ph | H | H | b2 | H |
| 2-4-670 | O | N—Ph | t-bu | t-bu | b2 | H |
| 2-4-671 | O | N—Ph | methyl | methyl | b2 | H |
| 2-4-672 | O | N—Ph | b1 | b1 | b2 | H |
| 2-4-673 | O | N—Ph | b2 | b2 | b2 | H |
| 2-4-674 | O | N—Ph | b3 | b3 | b2 | H |
| 2-4-675 | O | N—Ph | H | H | b3 | H |
| 2-4-676 | O | N—Ph | t-bu | t-bu | b3 | H |
| 2-4-677 | O | N—Ph | methyl | methyl | b3 | H |
| 2-4-678 | O | N—Ph | b1 | b1 | b3 | H |
| 2-4-679 | O | N—Ph | b2 | b2 | b3 | H |
| 2-4-680 | O | N—Ph | b3 | b3 | b3 | H |
| 2-4-681 | O | N—Ph | H | H | b4 | H |
| 2-4-682 | O | N—Ph | t-bu | t-bu | b4 | H |
| 2-4-683 | O | N—Ph | methyl | methyl | b4 | H |
| 2-4-684 | O | N—Ph | b1 | b1 | b4 | H |
| 2-4-685 | O | N—Ph | b2 | b2 | b4 | H |
| 2-4-686 | O | N—Ph | b3 | b3 | b4 | H |
| 2-4-687 | O | N—Ph | H | H | b5 | H |
| 2-4-688 | O | N—Ph | t-bu | t-bu | b5 | H |
| 2-4-689 | O | N—Ph | methyl | methyl | b5 | H |
| 2-4-690 | O | N—Ph | b1 | b1 | b5 | H |
| 2-4-691 | O | N—Ph | b2 | b2 | b5 | H |
| 2-4-692 | O | N—Ph | b3 | b3 | b5 | H |
| 2-4-693 | O | N—Ph | H | H | b6 | H |
| 2-4-694 | O | N—Ph | t-bu | t-bu | b6 | H |
| 2-4-695 | O | N—Ph | methyl | methyl | b6 | H |
| 2-4-696 | O | N—Ph | b1 | b1 | b6 | H |
| 2-4-697 | O | N—Ph | b2 | b2 | b6 | H |
| 2-4-698 | O | N—Ph | b3 | b3 | b6 | H |
| 2-4-699 | O | N—Ph | H | H | b7 | H |
| 2-4-700 | O | N—Ph | t-bu | t-bu | b7 | H |
| 2-4-701 | O | N—Ph | methyl | methyl | b7 | H |
| 2-4-702 | O | N—Ph | b1 | b1 | b7 | H |
| 2-4-703 | O | N—Ph | b2 | b2 | b7 | H |
| 2-4-704 | O | N—Ph | b3 | b3 | b7 | H |
| 2-4-705 | O | N—Ph | H | H | b8 | H |
| 2-4-706 | O | N—Ph | t-bu | t-bu | b8 | H |
| 2-4-707 | O | N—Ph | methyl | methyl | b8 | H |
| 2-4-708 | O | N—Ph | b1 | b1 | b8 | H |
| 2-4-709 | O | N—Ph | b2 | b2 | b8 | H |
| 2-4-710 | O | N—Ph | b3 | b3 | b8 | H |
| 2-4-711 | O | N—Ph | H | H | b9 | H |
| 2-4-712 | O | N—Ph | t-bu | t-bu | b9 | H |
| 2-4-713 | O | N—Ph | methyl | methyl | b9 | H |
| 2-4-714 | O | N—Ph | b1 | b1 | b9 | H |
| 2-4-715 | O | N—Ph | b2 | b2 | b9 | H |
| 2-4-716 | O | N—Ph | b3 | b3 | b9 | H |
| 2-4-717 | O | N—Ph | H | H | b10 | H |
| 2-4-718 | O | N—Ph | t-bu | t-bu | b10 | H |
| 2-4-719 | O | N—Ph | methyl | methyl | b10 | H |
| 2-4-720 | O | N—Ph | b1 | b1 | b10 | H |
| 2-4-721 | O | N—Ph | b2 | b2 | b10 | H |
| 2-4-722 | O | N—Ph | b3 | b3 | b10 | H |
| 2-4-723 | O | N—Ph | H | H | a1 | a1 |
| 2-4-724 | O | N—Ph | t-bu | t-bu | a1 | a1 |
| 2-4-725 | O | N—Ph | methyl | methyl | a1 | a1 |
| 2-4-726 | O | N—Ph | b1 | b1 | a1 | a1 |
| 2-4-727 | O | N—Ph | b2 | b2 | a1 | a1 |
| 2-4-728 | O | N—Ph | b3 | b3 | a1 | a1 |
| 2-4-729 | O | N—Ph | H | H | a2 | a2 |
| 2-4-730 | O | N—Ph | t-bu | t-bu | a2 | a2 |
| 2-4-731 | O | N—Ph | methyl | methyl | a2 | a2 |
| 2-4-732 | O | N—Ph | b1 | b1 | a2 | a2 |
| 2-4-733 | O | N—Ph | b2 | b2 | a2 | a2 |
| 2-4-734 | O | N—Ph | b3 | b3 | a2 | a2 |
| 2-4-735 | O | N—Ph | H | H | a3 | a3 |
| 2-4-736 | O | N—Ph | t-bu | t-bu | a3 | a3 |
| 2-4-737 | O | N—Ph | methyl | methyl | a3 | a3 |
| 2-4-738 | O | N—Ph | b1 | b1 | a3 | a3 |
| 2-4-739 | O | N—Ph | b2 | b2 | a3 | a3 |
| 2-4-740 | O | N—Ph | b3 | b3 | a3 | a3 |
| 2-4-741 | O | N—Ph | H | H | b1 | b1 |
| 2-4-742 | O | N—Ph | t-bu | t-bu | b1 | b1 |
| 2-4-743 | O | N—Ph | methyl | methyl | b1 | b1 |
| 2-4-744 | O | N—Ph | b1 | b1 | b1 | b1 |
| 2-4-745 | O | N—Ph | b2 | b2 | b1 | b1 |
| 2-4-746 | O | N—Ph | b3 | b3 | b1 | b1 |
| 2-4-747 | O | N—Ph | H | H | b2 | b2 |
| 2-4-748 | O | N—Ph | t-bu | t-bu | b2 | b2 |
| 2-4-749 | O | N—Ph | methyl | methyl | b2 | b2 |
| 2-4-750 | O | N—Ph | b1 | b1 | b2 | b2 |
| 2-4-751 | O | N—Ph | b2 | b2 | b2 | b2 |
| 2-4-752 | O | N—Ph | b3 | b3 | b2 | b2 |
| 2-4-753 | O | N—Ph | H | H | b3 | b3 |
| 2-4-754 | O | N—Ph | t-bu | t-bu | b3 | b3 |
| 2-4-755 | O | N—Ph | methyl | methyl | b3 | b3 |
| 2-4-756 | O | N—Ph | b1 | b1 | b3 | b3 |
| 2-4-757 | O | N—Ph | b2 | b2 | b3 | b3 |
| 2-4-758 | O | N—Ph | b3 | b3 | b3 | b3 |
| 2-4-759 | O | N—Ph | H | H | b5 | b5 |
| 2-4-760 | O | N—Ph | t-bu | t-bu | b5 | b5 |
| 2-4-761 | O | N—Ph | methyl | methyl | b5 | b5 |
| 2-4-762 | O | N—Ph | b1 | b1 | b5 | b5 |
| 2-4-763 | O | N—Ph | b2 | b2 | b5 | b5 |
| 2-4-764 | O | N—Ph | b3 | b3 | b5 | b5 |
| 2-4-765 | O | N—Ph | H | H | b6 | b6 |
| 2-4-766 | O | N—Ph | t-bu | t-bu | b6 | b6 |
| 2-4-767 | O | N—Ph | methyl | methyl | b6 | b6 |
| 2-4-768 | O | N—Ph | b1 | b1 | b6 | b6 |
| 2-4-769 | O | N—Ph | b2 | b2 | b6 | b6 |
| 2-4-770 | O | N—Ph | b3 | b3 | b6 | b6 |
| 2-4-771 | O | N—Ph | H | H | b7 | b7 |
| 2-4-772 | O | N—Ph | t-bu | t-bu | b7 | b7 |
| 2-4-773 | O | N—Ph | methyl | methyl | b7 | b7 |
| 2-4-774 | O | N—Ph | b1 | b1 | b7 | b7 |
| 2-4-775 | O | N—Ph | b2 | b2 | b7 | b7 |
| 2-4-776 | O | N—Ph | b3 | b3 | b7 | b7 |
| 2-4-777 | S | N—Ph | H | H | H | H |
| 2-4-778 | S | N—Ph | t-bu | t-bu | H | H |
| 2-4-779 | S | N—Ph | methyl | methyl | H | H |
| 2-4-780 | S | N—Ph | t-bu | t-bu | t-bu | t-bu |
| 2-4-781 | S | N—Ph | methyl | methyl | methyl | methyl |
| 2-4-782 | S | N—Ph | b1 | b1 | H | H |
| 2-4-783 | S | N—Ph | b2 | b2 | H | H |
| 2-4-784 | S | N—Ph | b3 | b3 | H | H |
| 2-4-785 | S | N—Ph | H | H | H | a1 |
| 2-4-786 | S | N—Ph | t-bu | t-bu | H | a1 |
| 2-4-787 | S | N—Ph | methyl | methyl | H | a1 |
| 2-4-788 | S | N—Ph | b1 | b1 | H | a1 |
| 2-4-789 | S | N—Ph | b2 | b2 | H | a1 |
| 2-4-790 | S | N—Ph | b3 | b3 | H | a1 |
| 2-4-791 | S | N—Ph | H | H | H | a2 |
| 2-4-792 | S | N—Ph | t-bu | t-bu | H | a2 |
| 2-4-793 | S | N—Ph | methyl | methyl | H | a2 |
| 2-4-794 | S | N—Ph | b1 | b1 | H | a2 |
| 2-4-795 | S | N—Ph | b2 | b2 | H | a2 |
| 2-4-796 | S | N—Ph | b3 | b3 | H | a2 |
| 2-4-797 | S | N—Ph | H | H | H | a3 |
| 2-4-798 | S | N—Ph | t-bu | t-bu | H | a3 |
| 2-4-799 | S | N—Ph | methyl | methyl | H | a3 |
| 2-4-800 | S | N—Ph | b1 | b1 | H | a3 |
| 2-4-801 | S | N—Ph | b2 | b2 | H | a3 |
| 2-4-802 | S | N—Ph | b3 | b3 | H | a3 |
| 2-4-803 | S | N—Ph | H | H | H | a4 |
| 2-4-804 | S | N—Ph | t-bu | t-bu | H | a4 |
| 2-4-805 | S | N—Ph | methyl | methyl | H | a4 |
| 2-4-806 | S | N—Ph | b1 | b1 | H | a4 |
| 2-4-807 | S | N—Ph | b2 | b2 | H | a4 |
| 2-4-808 | S | N—Ph | b3 | b3 | H | a4 |
| 2-4-809 | S | N—Ph | H | H | H | a5 |
| 2-4-810 | S | N—Ph | t-bu | t-bu | H | a5 |
| 2-4-811 | S | N—Ph | methyl | methyl | H | a5 |
| 2-4-812 | S | N—Ph | b1 | b1 | H | a5 |
| 2-4-813 | S | N—Ph | b2 | b2 | H | a5 |
| 2-4-814 | S | N—Ph | b3 | b3 | H | a5 |
| 2-4-815 | S | N—Ph | H | H | H | a6 |
| 2-4-816 | S | N—Ph | t-bu | t-bu | H | a6 |
| 2-4-817 | S | N—Ph | methyl | methyl | H | a6 |
| 2-4-818 | S | N—Ph | b1 | b1 | H | a6 |
| 2-4-819 | S | N—Ph | b2 | b2 | H | a6 |
| 2-4-820 | S | N—Ph | b3 | b3 | H | a6 |
| 2-4-821 | S | N—Ph | H | H | a1 | H |
| 2-4-822 | S | N—Ph | t-bu | t-bu | a1 | H |
| 2-4-823 | S | N—Ph | methyl | methyl | a1 | H |
| 2-4-824 | S | N—Ph | b1 | b1 | a1 | H |
| 2-4-825 | S | N—Ph | b2 | b2 | a1 | H |
| 2-4-826 | S | N—Ph | b3 | b3 | a1 | H |
| 2-4-827 | S | N—Ph | H | H | a2 | H |
| 2-4-828 | S | N—Ph | t-bu | t-bu | a2 | H |
| 2-4-829 | S | N—Ph | methyl | methyl | a2 | H |
| 2-4-830 | S | N—Ph | b1 | b1 | a2 | H |
| 2-4-831 | S | N—Ph | b2 | b2 | a2 | H |
| 2-4-832 | S | N—Ph | b3 | b3 | a2 | H |
| 2-4-833 | S | N—Ph | H | H | a3 | H |
| 2-4-834 | S | N—Ph | t-bu | t-bu | a3 | H |
| 2-4-835 | S | N—Ph | methyl | methyl | a3 | H |
| 2-4-836 | S | N—Ph | b1 | b1 | a3 | H |
| 2-4-837 | S | N—Ph | b2 | b2 | a3 | H |
| 2-4-838 | S | N—Ph | b3 | b3 | a3 | H |
| 2-4-839 | S | N—Ph | H | H | a4 | H |
| 2-4-840 | S | N—Ph | t-bu | t-bu | a4 | H |
| 2-4-841 | S | N—Ph | methyl | methyl | a4 | H |
| 2-4-842 | S | N—Ph | b1 | b1 | a4 | H |
| 2-4-843 | S | N—Ph | b2 | b2 | a4 | H |
| 2-4-844 | S | N—Ph | b3 | b3 | a4 | H |
| 2-4-845 | S | N—Ph | H | H | a5 | H |
| 2-4-846 | S | N—Ph | t-bu | t-bu | a5 | H |
| 2-4-847 | S | N—Ph | methyl | methyl | a5 | H |
| 2-4-848 | S | N—Ph | b1 | b1 | a5 | H |
| 2-4-849 | S | N—Ph | b2 | b2 | a5 | H |
| 2-4-850 | S | N—Ph | b3 | b3 | a5 | H |
| 2-4-851 | S | N—Ph | H | H | a6 | H |
| 2-4-852 | S | N—Ph | t-bu | t-bu | a6 | H |
| 2-4-853 | S | N—Ph | methyl | methyl | a6 | H |
| 2-4-854 | S | N—Ph | b1 | b1 | a6 | H |
| 2-4-855 | S | N—Ph | b2 | b2 | a6 | H |
| 2-4-856 | S | N—Ph | b3 | b3 | a6 | H |
| 2-4-857 | S | N—Ph | H | H | b1 | H |
| 2-4-858 | S | N—Ph | t-bu | t-bu | b1 | H |
| 2-4-859 | S | N—Ph | methyl | methyl | b1 | H |
| 2-4-860 | S | N—Ph | b1 | b1 | b1 | H |
| 2-4-861 | S | N—Ph | b2 | b2 | b1 | H |
| 2-4-862 | S | N—Ph | b3 | b3 | b1 | H |
| 2-4-863 | S | N—Ph | H | H | b2 | H |
| 2-4-864 | S | N—Ph | t-bu | t-bu | b2 | H |
| 2-4-865 | S | N—Ph | methyl | methyl | b2 | H |
| 2-4-866 | S | N—Ph | b1 | b1 | b2 | H |
| 2-4-867 | S | N—Ph | b2 | b2 | b2 | H |
| 2-4-868 | S | N—Ph | b3 | b3 | b2 | H |
| 2-4-869 | S | N—Ph | H | H | b3 | H |
| 2-4-870 | S | N—Ph | t-bu | t-bu | b3 | H |
| 2-4-871 | S | N—Ph | methyl | methyl | b3 | H |
| 2-4-872 | S | N—Ph | b1 | b1 | b3 | H |
| 2-4-873 | S | N—Ph | b2 | b2 | b3 | H |
| 2-4-874 | S | N—Ph | b3 | b3 | b3 | H |
| 2-4-875 | S | N—Ph | H | H | b4 | H |
| 2-4-876 | S | N—Ph | t-bu | t-bu | b4 | H |
| 2-4-877 | S | N—Ph | methyl | methyl | b4 | H |
| 2-4-878 | S | N—Ph | b1 | b1 | b4 | H |
| 2-4-879 | S | N—Ph | b2 | b2 | b4 | H |
| 2-4-880 | S | N—Ph | b3 | b3 | b4 | H |
| 2-4-881 | S | N—Ph | H | H | b5 | H |
| 2-4-882 | S | N—Ph | t-bu | t-bu | b5 | H |
| 2-4-883 | S | N—Ph | methyl | methyl | b5 | H |
| 2-4-884 | S | N—Ph | b1 | b1 | b5 | H |
| 2-4-885 | S | N—Ph | b2 | b2 | b5 | H |
| 2-4-886 | S | N—Ph | b3 | b3 | b5 | H |
| 2-4-887 | S | N—Ph | H | H | b6 | H |
| 2-4-888 | S | N—Ph | t-bu | t-bu | b6 | H |
| 2-4-889 | S | N—Ph | methyl | methyl | b6 | H |
| 2-4-890 | S | N—Ph | b1 | b1 | b6 | H |
| 2-4-891 | S | N—Ph | b2 | b2 | b6 | H |
| 2-4-892 | S | N—Ph | b3 | b3 | b6 | H |
| 2-4-893 | S | N—Ph | H | H | b7 | H |
| 2-4-894 | S | N—Ph | t-bu | t-bu | b7 | H |
| 2-4-895 | S | N—Ph | methyl | methyl | b7 | H |
| 2-4-896 | S | N—Ph | b1 | b1 | b7 | H |
| 2-4-897 | S | N—Ph | b2 | b2 | b7 | H |
| 2-4-898 | S | N—Ph | b3 | b3 | b7 | H |
| 2-4-899 | S | N—Ph | H | H | b8 | H |
| 2-4-900 | S | N—Ph | t-bu | t-bu | b8 | H |
| 2-4-901 | S | N—Ph | methyl | methyl | b8 | H |
| 2-4-902 | S | N—Ph | b1 | b1 | b8 | H |
| 2-4-903 | S | N—Ph | b2 | b2 | b8 | H |
| 2-4-904 | S | N—Ph | b3 | b3 | b8 | H |
| 2-4-905 | S | N—Ph | H | H | b9 | H |
| 2-4-906 | S | N—Ph | t-bu | t-bu | b9 | H |
| 2-4-907 | S | N—Ph | methyl | methyl | b9 | H |
| 2-4-908 | S | N—Ph | b1 | b1 | b9 | H |
| 2-4-909 | S | N—Ph | b2 | b2 | b9 | H |
| 2-4-910 | S | N—Ph | b3 | b3 | b9 | H |
| 2-4-911 | S | N—Ph | H | H | b10 | H |
| 2-4-912 | S | N—Ph | t-bu | t-bu | b10 | H |
| 2-4-913 | S | N—Ph | methyl | methyl | b10 | H |
| 2-4-914 | S | N—Ph | b1 | b1 | b10 | H |
| 2-4-915 | S | N—Ph | b2 | b2 | b10 | H |
| 2-4-916 | S | N—Ph | b3 | b3 | b10 | H |
| 2-4-917 | S | N—Ph | H | H | a1 | a1 |
| 2-4-918 | S | N—Ph | t-bu | t-bu | a1 | a1 |
| 2-4-919 | S | N—Ph | methyl | methyl | a1 | a1 |
| 2-4-920 | S | N—Ph | b1 | b1 | a1 | a1 |
| 2-4-921 | S | N—Ph | b2 | b2 | a1 | a1 |
| 2-4-922 | S | N—Ph | b3 | b3 | a1 | a1 |
| 2-4-923 | S | N—Ph | H | H | a2 | a2 |
| 2-4-924 | S | N—Ph | t-bu | t-bu | a2 | a2 |
| 2-4-925 | S | N—Ph | methyl | methyl | a2 | a2 |
| 2-4-926 | S | N—Ph | b1 | b1 | a2 | a2 |
| 2-4-927 | S | N—Ph | b2 | b2 | a2 | a2 |
| 2-4-928 | S | N—Ph | b3 | b3 | a2 | a2 |
| 2-4-929 | S | N—Ph | H | H | a3 | a3 |
| 2-4-930 | S | N—Ph | t-bu | t-bu | a3 | a3 |
| 2-4-931 | S | N—Ph | methyl | methyl | a3 | a3 |
| 2-4-932 | S | N—Ph | b1 | b1 | a3 | a3 |
| 2-4-933 | S | N—Ph | b2 | b2 | a3 | a3 |
| 2-4-934 | S | N—Ph | b3 | b3 | a3 | a3 |
| 2-4-935 | S | N—Ph | H | H | b1 | b1 |
| 2-4-936 | S | N—Ph | t-bu | t-bu | b1 | b1 |
| 2-4-937 | S | N—Ph | methyl | methyl | b1 | b1 |
| 2-4-938 | S | N—Ph | b1 | b1 | b1 | b1 |
| 2-4-939 | S | N—Ph | b2 | b2 | b1 | b1 |
| 2-4-940 | S | N—Ph | b3 | b3 | b1 | b1 |
| 2-4-941 | S | N—Ph | H | H | b2 | b2 |
| 2-4-942 | S | N—Ph | t-bu | t-bu | b2 | b2 |
| 2-4-943 | S | N—Ph | methyl | methyl | b2 | b2 |
| 2-4-944 | S | N—Ph | b1 | b1 | b2 | b2 |
| 2-4-945 | S | N—Ph | b2 | b2 | b2 | b2 |
| 2-4-946 | S | N—Ph | b3 | b3 | b2 | b2 |
| 2-4-947 | S | N—Ph | H | H | b3 | b3 |
| 2-4-948 | S | N—Ph | t-bu | t-bu | b3 | b3 |
| 2-4-949 | S | N—Ph | methyl | methyl | b3 | b3 |
| 2-4-950 | S | N—Ph | b1 | b1 | b3 | b3 |
| 2-4-951 | S | N—Ph | b2 | b2 | b3 | b3 |
| 2-4-952 | S | N—Ph | b3 | b3 | b3 | b3 |
| 2-4-953 | S | N—Ph | H | H | b5 | b5 |
| 2-4-954 | S | N—Ph | t-bu | t-bu | b5 | b5 |
| 2-4-955 | S | N—Ph | methyl | methyl | b5 | b5 |
| 2-4-956 | S | N—Ph | b1 | b1 | b5 | b5 |
| 2-4-957 | S | N—Ph | b2 | b2 | b5 | b5 |
| 2-4-958 | S | N—Ph | b3 | b3 | b5 | b5 |
| 2-4-959 | S | N—Ph | H | H | b6 | b6 |
| 2-4-960 | S | N—Ph | t-bu | t-bu | b6 | b6 |
| 2-4-961 | S | N—Ph | methyl | methyl | b6 | b6 |
| 2-4-962 | S | N—Ph | b1 | b1 | b6 | b6 |
| 2-4-963 | S | N—Ph | b2 | b2 | b6 | b6 |
| 2-4-964 | S | N—Ph | b3 | b3 | b6 | b6 |
| 2-4-965 | S | N—Ph | H | H | b7 | b7 |
| 2-4-966 | S | N—Ph | t-bu | t-bu | b7 | b7 |
| 2-4-967 | S | N—Ph | methyl | methyl | b7 | b7 |
| 2-4-968 | S | N—Ph | b1 | b1 | b7 | b7 |
| 2-4-969 | S | N—Ph | b2 | b2 | b7 | b7 |
| 2-4-970 | S | N—Ph | b3 | b3 | b7 | b7 |

### Group 2-5

The compounds are represented by the formula 2-5 below, wherein CY₁ and CY₂ are each a benzene group, X₁ is B, and Y1, Y2, R3a, (R1)a1, (R2)a2 are each shown in the table 5 below,

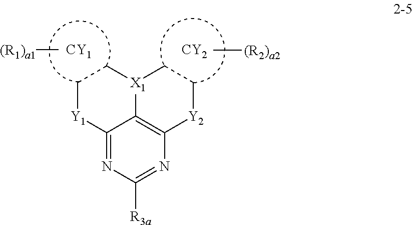

TABLE 5

| Compound | | | | | |
|---|---|---|---|---|---|
| No. | Y1 | Y2 | R3a | (R1)a1 | (R2)a2 |
| 2-5-001 | N—Ph | N—Ph | H | H | H |
| 2-5-002 | N—Ph | N—Ph | t-bu | H | H |
| 2-5-003 | N—Ph | N—Ph | methyl | H | H |
| 2-5-004 | N—Ph | N—Ph | t-bu | t-bu | t-bu |
| 2-5-005 | N—Ph | N—Ph | methyl | methyl | methyl |
| 2-5-006 | N—Ph | N—Ph | b1 | H | H |
| 2-5-007 | N—Ph | N—Ph | b2 | H | H |
| 2-5-008 | N—Ph | N—Ph | b3 | H | H |
| 2-5-009 | N—Ph | N—Ph | H | H | a1 |
| 2-5-010 | N—Ph | N—Ph | t-bu | H | a1 |
| 2-5-011 | N—Ph | N—Ph | methyl | H | a1 |
| 2-5-012 | N—Ph | N—Ph | b1 | H | a1 |
| 2-5-013 | N—Ph | N—Ph | b2 | H | a1 |
| 2-5-014 | N—Ph | N—Ph | b3 | H | a1 |
| 2-5-015 | N—Ph | N—Ph | H | H | a2 |
| 2-5-016 | N—Ph | N—Ph | t-bu | H | a2 |
| 2-5-017 | N—Ph | N—Ph | methyl | H | a2 |
| 2-5-018 | N—Ph | N—Ph | b1 | H | a2 |
| 2-5-019 | N—Ph | N—Ph | b2 | H | a2 |
| 2-5-020 | N—Ph | N—Ph | b3 | H | a2 |
| 2-5-021 | N—Ph | N—Ph | H | H | a3 |
| 2-5-022 | N—Ph | N—Ph | t-bu | H | a3 |
| 2-5-023 | N—Ph | N—Ph | methyl | H | a3 |
| 2-5-024 | N—Ph | N—Ph | b1 | H | a3 |
| 2-5-025 | N—Ph | N—Ph | b2 | H | a3 |
| 2-5-026 | N—Ph | N—Ph | b3 | H | a3 |
| 2-5-027 | N—Ph | N—Ph | H | H | a4 |
| 2-5-028 | N—Ph | N—Ph | t-bu | H | a4 |
| 2-5-029 | N—Ph | N—Ph | methyl | H | a4 |
| 2-5-030 | N—Ph | N—Ph | b1 | H | a4 |
| 2-5-031 | N—Ph | N—Ph | b2 | H | a4 |
| 2-5-032 | N—Ph | N—Ph | b3 | H | a4 |
| 2-5-033 | N—Ph | N—Ph | H | H | a5 |
| 2-5-034 | N—Ph | N—Ph | t-bu | H | a5 |
| 2-5-035 | N—Ph | N—Ph | methyl | H | a5 |
| 2-5-036 | N—Ph | N—Ph | b1 | H | a5 |
| 2-5-037 | N—Ph | N—Ph | b2 | H | a5 |
| 2-5-038 | N—Ph | N—Ph | b3 | H | a5 |
| 2-5-039 | N—Ph | N—Ph | H | H | a6 |
| 2-5-040 | N—Ph | N—Ph | t-bu | H | a6 |
| 2-5-041 | N—Ph | N—Ph | methyl | H | a6 |
| 2-5-042 | N—Ph | N—Ph | b1 | H | a6 |
| 2-5-043 | N—Ph | N—Ph | b2 | H | a6 |
| 2-5-044 | N—Ph | N—Ph | b3 | H | a6 |
| 2-5-045 | N—Ph | N—Ph | H | H | b1 |
| 2-5-046 | N—Ph | N—Ph | t-bu | H | b1 |
| 2-5-047 | N—Ph | N—Ph | methyl | H | b1 |
| 2-5-048 | N—Ph | N—Ph | b1 | H | b1 |
| 2-5-049 | N—Ph | N—Ph | b2 | H | b1 |
| 2-5-050 | N—Ph | N—Ph | b3 | H | b1 |
| 2-5-051 | N—Ph | N—Ph | H | H | b2 |
| 2-5-052 | N—Ph | N—Ph | t-bu | H | b2 |
| 2-5-053 | N—Ph | N—Ph | methyl | H | b2 |
| 2-5-054 | N—Ph | N—Ph | b1 | H | b2 |
| 2-5-055 | N—Ph | N—Ph | b2 | H | b2 |
| 2-5-056 | N—Ph | N—Ph | b3 | H | b2 |
| 2-5-057 | N—Ph | N—Ph | H | H | b3 |
| 2-5-058 | N—Ph | N—Ph | t-bu | H | b3 |
| 2-5-059 | N—Ph | N—Ph | methyl | H | b3 |
| 2-5-060 | N—Ph | N—Ph | b1 | H | b3 |
| 2-5-061 | N—Ph | N—Ph | b2 | H | b3 |
| 2-5-062 | N—Ph | N—Ph | b3 | H | b3 |
| 2-5-063 | N—Ph | N—Ph | H | H | b4 |
| 2-5-064 | N—Ph | N—Ph | t-bu | H | b4 |
| 2-5-065 | N—Ph | N—Ph | methyl | H | b4 |
| 2-5-066 | N—Ph | N—Ph | b1 | H | b4 |
| 2-5-067 | N—Ph | N—Ph | b2 | H | b4 |
| 2-5-068 | N—Ph | N—Ph | b3 | H | b4 |
| 2-5-069 | N—Ph | N—Ph | H | H | b5 |
| 2-5-070 | N—Ph | N—Ph | t-bu | H | b5 |
| 2-5-071 | N—Ph | N—Ph | methyl | H | b5 |
| 2-5-072 | N—Ph | N—Ph | b1 | H | b5 |
| 2-5-073 | N—Ph | N—Ph | b2 | H | b5 |
| 2-5-074 | N—Ph | N—Ph | b3 | H | b5 |
| 2-5-075 | N—Ph | N—Ph | H | H | b6 |
| 2-5-076 | N—Ph | N—Ph | t-bu | H | b6 |
| 2-5-077 | N—Ph | N—Ph | methyl | H | b6 |
| 2-5-078 | N—Ph | N—Ph | b1 | H | b6 |
| 2-5-079 | N—Ph | N—Ph | b2 | H | b6 |
| 2-5-080 | N—Ph | N—Ph | b3 | H | b6 |
| 2-5-081 | N—Ph | N—Ph | H | H | b7 |
| 2-5-082 | N—Ph | N—Ph | t-bu | H | b7 |
| 2-5-083 | N—Ph | N—Ph | methyl | H | b7 |
| 2-5-084 | N—Ph | N—Ph | b1 | H | b7 |
| 2-5-085 | N—Ph | N—Ph | b2 | H | b7 |
| 2-5-086 | N—Ph | N—Ph | b3 | H | b7 |
| 2-5-087 | N—Ph | N—Ph | H | H | b8 |
| 2-5-088 | N—Ph | N—Ph | t-bu | H | b8 |
| 2-5-089 | N—Ph | N—Ph | methyl | H | b8 |
| 2-5-090 | N—Ph | N—Ph | b1 | H | b8 |
| 2-5-091 | N—Ph | N—Ph | b2 | H | b8 |
| 2-5-092 | N—Ph | N—Ph | b3 | H | b8 |
| 2-5-093 | N—Ph | N—Ph | H | H | b9 |
| 2-5-094 | N—Ph | N—Ph | t-bu | H | b9 |
| 2-5-095 | N—Ph | N—Ph | methyl | H | b9 |
| 2-5-096 | N—Ph | N—Ph | b1 | H | b9 |
| 2-5-097 | N—Ph | N—Ph | b2 | H | b9 |
| 2-5-098 | N—Ph | N—Ph | b3 | H | b9 |
| 2-5-099 | N—Ph | N—Ph | H | H | b10 |
| 2-5-100 | N—Ph | N—Ph | t-bu | H | b10 |
| 2-5-101 | N—Ph | N—Ph | methyl | H | b10 |
| 2-5-102 | N—Ph | N—Ph | b1 | H | b10 |
| 2-5-103 | N—Ph | N—Ph | b2 | H | b10 |
| 2-5-104 | N—Ph | N—Ph | b3 | H | b10 |
| 2-5-105 | N—Ph | N—Ph | H | a1 | H |
| 2-5-106 | N—Ph | N—Ph | t-bu | a1 | H |
| 2-5-107 | N—Ph | N—Ph | methyl | a1 | H |
| 2-5-108 | N—Ph | N—Ph | b1 | a1 | H |
| 2-5-109 | N—Ph | N—Ph | b2 | a1 | H |
| 2-5-110 | N—Ph | N—Ph | b3 | a1 | H |
| 2-5-111 | N—Ph | N—Ph | H | a2 | H |
| 2-5-112 | N—Ph | N—Ph | t-bu | a2 | H |
| 2-5-113 | N—Ph | N—Ph | methyl | a2 | H |
| 2-5-114 | N—Ph | N—Ph | b1 | a2 | H |
| 2-5-115 | N—Ph | N—Ph | b2 | a2 | H |
| 2-5-116 | N—Ph | N—Ph | b3 | a2 | H |
| 2-5-117 | N—Ph | N—Ph | H | a3 | H |
| 2-5-118 | N—Ph | N—Ph | t-bu | a3 | H |
| 2-5-119 | N—Ph | N—Ph | methyl | a3 | H |
| 2-5-120 | N—Ph | N—Ph | b1 | a3 | H |
| 2-5-121 | N—Ph | N—Ph | b2 | a3 | H |
| 2-5-122 | N—Ph | N—Ph | b3 | a3 | H |
| 2-5-123 | N—Ph | N—Ph | H | a4 | H |
| 2-5-124 | N—Ph | N—Ph | t-bu | a4 | H |
| 2-5-125 | N—Ph | N—Ph | methyl | a4 | H |
| 2-5-126 | N—Ph | N—Ph | b1 | a4 | H |
| 2-5-127 | N—Ph | N—Ph | b2 | a4 | H |
| 2-5-128 | N—Ph | N—Ph | b3 | a4 | H |
| 2-5-129 | N—Ph | N—Ph | H | a5 | H |
| 2-5-130 | N—Ph | N—Ph | t-bu | a5 | H |
| 2-5-131 | N—Ph | N—Ph | methyl | a5 | H |
| 2-5-132 | N—Ph | N—Ph | b1 | a5 | H |
| 2-5-133 | N—Ph | N—Ph | b2 | a5 | H |
| 2-5-134 | N—Ph | N—Ph | b3 | a5 | H |
| 2-5-135 | N—Ph | N—Ph | H | a6 | H |
| 2-5-136 | N—Ph | N—Ph | t-bu | a6 | H |
| 2-5-137 | N—Ph | N—Ph | methyl | a6 | H |
| 2-5-138 | N—Ph | N—Ph | b1 | a6 | H |
| 2-5-139 | N—Ph | N—Ph | b2 | a6 | H |
| 2-5-140 | N—Ph | N—Ph | b3 | a6 | H |
| 2-5-141 | N—Ph | N—Ph | H | b1 | H |
| 2-5-142 | N—Ph | N—Ph | t-bu | b1 | H |
| 2-5-143 | N—Ph | N—Ph | methyl | b1 | H |
| 2-5-144 | N—Ph | N—Ph | b1 | b1 | H |
| 2-5-145 | N—Ph | N—Ph | b2 | b1 | H |
| 2-5-146 | N—Ph | N—Ph | b3 | b1 | H |
| 2-5-147 | N—Ph | N—Ph | H | b2 | H |
| 2-5-148 | N—Ph | N—Ph | t-bu | b2 | H |
| 2-5-149 | N—Ph | N—Ph | methyl | b2 | H |
| 2-5-150 | N—Ph | N—Ph | b1 | b2 | H |
| 2-5-151 | N—Ph | N—Ph | b2 | b2 | H |
| 2-5-152 | N—Ph | N—Ph | b3 | b2 | H |
| 2-5-153 | N—Ph | N—Ph | H | b3 | H |
| 2-5-154 | N—Ph | N—Ph | t-bu | b3 | H |
| 2-5-155 | N—Ph | N—Ph | methyl | b3 | H |
| 2-5-156 | N—Ph | N—Ph | b1 | b3 | H |
| 2-5-157 | N—Ph | N—Ph | b2 | b3 | H |
| 2-5-158 | N—Ph | N—Ph | b3 | b3 | H |
| 2-5-159 | N—Ph | N—Ph | H | b4 | H |
| 2-5-160 | N—Ph | N—Ph | t-bu | b4 | H |
| 2-5-161 | N—Ph | N—Ph | methyl | b4 | H |
| 2-5-162 | N—Ph | N—Ph | b1 | b4 | H |
| 2-5-163 | N—Ph | N—Ph | b2 | b4 | H |
| 2-5-164 | N—Ph | N—Ph | b3 | b4 | H |
| 2-5-165 | N—Ph | N—Ph | H | b5 | H |
| 2-5-166 | N—Ph | N—Ph | t-bu | b5 | H |
| 2-5-167 | N—Ph | N—Ph | methyl | b5 | H |
| 2-5-168 | N—Ph | N—Ph | b1 | b5 | H |
| 2-5-169 | N—Ph | N—Ph | b2 | b5 | H |
| 2-5-170 | N—Ph | N—Ph | b3 | b5 | H |
| 2-5-171 | N—Ph | N—Ph | H | b6 | H |
| 2-5-172 | N—Ph | N—Ph | t-bu | b6 | H |
| 2-5-173 | N—Ph | N—Ph | methyl | b6 | H |
| 2-5-174 | N—Ph | N—Ph | b1 | b6 | H |
| 2-5-175 | N—Ph | N—Ph | b2 | b6 | H |
| 2-5-176 | N—Ph | N—Ph | b3 | b6 | H |
| 2-5-177 | N—Ph | N—Ph | H | b7 | H |
| 2-5-178 | N—Ph | N—Ph | t-bu | b7 | H |
| 2-5-179 | N—Ph | N—Ph | methyl | b7 | H |
| 2-5-180 | N—Ph | N—Ph | b1 | b7 | H |
| 2-5-181 | N—Ph | N—Ph | b2 | b7 | H |
| 2-5-182 | N—Ph | N—Ph | b3 | b7 | H |
| 2-5-183 | N—Ph | N—Ph | H | b8 | H |
| 2-5-184 | N—Ph | N—Ph | t-bu | b8 | H |
| 2-5-185 | N—Ph | N—Ph | methyl | b8 | H |
| 2-5-186 | N—Ph | N—Ph | b1 | b8 | H |
| 2-5-187 | N—Ph | N—Ph | b2 | b8 | H |
| 2-5-188 | N—Ph | N—Ph | b3 | b8 | H |
| 2-5-189 | N—Ph | N—Ph | H | b9 | H |
| 2-5-190 | N—Ph | N—Ph | t-bu | b9 | H |
| 2-5-191 | N—Ph | N—Ph | methyl | b9 | H |
| 2-5-192 | N—Ph | N—Ph | b1 | b9 | H |
| 2-5-193 | N—Ph | N—Ph | b2 | b9 | H |
| 2-5-194 | N—Ph | N—Ph | b3 | b9 | H |
| 2-5-195 | N—Ph | N—Ph | H | b10 | H |
| 2-5-196 | N—Ph | N—Ph | t-bu | b10 | H |
| 2-5-197 | N—Ph | N—Ph | methyl | b10 | H |
| 2-5-198 | N—Ph | N—Ph | b1 | b10 | H |
| 2-5-199 | N—Ph | N—Ph | b2 | b10 | H |
| 2-5-200 | N—Ph | N—Ph | b3 | b10 | H |
| 2-5-201 | N—Ph | N—Ph | H | a1 | a1 |
| 2-5-202 | N—Ph | N—Ph | t-bu | a1 | a1 |
| 2-5-203 | N—Ph | N—Ph | methyl | a1 | a1 |
| 2-5-204 | N—Ph | N—Ph | b1 | a1 | a1 |
| 2-5-205 | N—Ph | N—Ph | b2 | a1 | a1 |
| 2-5-206 | N—Ph | N—Ph | b3 | a1 | a1 |
| 2-5-207 | N—Ph | N—Ph | H | a2 | a2 |
| 2-5-208 | N—Ph | N—Ph | t-bu | a2 | a2 |
| 2-5-209 | N—Ph | N—Ph | methyl | a2 | a2 |
| 2-5-210 | N—Ph | N—Ph | b1 | a2 | a2 |
| 2-5-211 | N—Ph | N—Ph | b2 | a2 | a2 |
| 2-5-212 | N—Ph | N—Ph | b3 | a2 | a2 |
| 2-5-213 | N—Ph | N—Ph | H | a3 | a3 |
| 2-5-214 | N—Ph | N—Ph | t-bu | a3 | a3 |
| 2-5-215 | N—Ph | N—Ph | methyl | a3 | a3 |
| 2-5-216 | N—Ph | N—Ph | b1 | a3 | a3 |
| 2-5-217 | N—Ph | N—Ph | b2 | a3 | a3 |
| 2-5-218 | N—Ph | N—Ph | b3 | a3 | a3 |
| 2-5-219 | N—Ph | N—Ph | H | b1 | b1 |
| 2-5-220 | N—Ph | N—Ph | t-bu | b1 | b1 |
| 2-5-221 | N—Ph | N—Ph | methyl | b1 | b1 |
| 2-5-222 | N—Ph | N—Ph | b1 | b1 | b1 |
| 2-5-223 | N—Ph | N—Ph | b2 | b1 | b1 |
| 2-5-224 | N—Ph | N—Ph | b3 | b1 | b1 |
| 2-5-225 | N—Ph | N—Ph | H | b2 | b2 |
| 2-5-226 | N—Ph | N—Ph | t-bu | b2 | b2 |
| 2-5-227 | N—Ph | N—Ph | methyl | b2 | b2 |
| 2-5-228 | N—Ph | N—Ph | b1 | b2 | b2 |
| 2-5-229 | N—Ph | N—Ph | b2 | b2 | b2 |
| 2-5-230 | N—Ph | N—Ph | b3 | b2 | b2 |
| 2-5-231 | N—Ph | N—Ph | H | b3 | b3 |
| 2-5-232 | N—Ph | N—Ph | t-bu | b3 | b3 |
| 2-5-233 | N—Ph | N—Ph | methyl | b3 | b3 |
| 2-5-234 | N—Ph | N—Ph | b1 | b3 | b3 |
| 2-5-235 | N—Ph | N—Ph | b2 | b3 | b3 |
| 2-5-236 | N—Ph | N—Ph | b3 | b3 | b3 |
| 2-5-237 | N—Ph | N—Ph | H | b5 | b5 |
| 2-5-238 | N—Ph | N—Ph | t-bu | b5 | b5 |
| 2-5-239 | N—Ph | N—Ph | methyl | b5 | b5 |
| 2-5-240 | N—Ph | N—Ph | b1 | b5 | b5 |
| 2-5-241 | N—Ph | N—Ph | b2 | b5 | b5 |
| 2-5-242 | N—Ph | N—Ph | b3 | b5 | b5 |
| 2-5-243 | N—Ph | N—Ph | H | b6 | b6 |
| 2-5-244 | N—Ph | N—Ph | t-bu | b6 | b6 |
| 2-5-245 | N—Ph | N—Ph | methyl | b6 | b6 |
| 2-5-246 | N—Ph | N—Ph | b1 | b6 | b6 |
| 2-5-247 | N—Ph | N—Ph | b2 | b6 | b6 |
| 2-5-248 | N—Ph | N—Ph | b3 | b6 | b6 |
| 2-5-249 | N—Ph | N—Ph | H | b7 | b7 |
| 2-5-250 | N—Ph | N—Ph | t-bu | b7 | b7 |
| 2-5-251 | N—Ph | N—Ph | methyl | b7 | b7 |
| 2-5-252 | N—Ph | N—Ph | b1 | b7 | b7 |
| 2-5-253 | N—Ph | N—Ph | b2 | b7 | b7 |
| 2-5-254 | N—Ph | N—Ph | b3 | b7 | b7 |
| 2-5-255 | N-tolyl | N-tolyl | H | H | H |
| 2-5-256 | N-tolyl | N-tolyl | t-bu | H | H |
| 2-5-257 | N-tolyl | N-tolyl | methyl | H | H |
| 2-5-258 | N-tolyl | N-tolyl | t-bu | t-bu | t-bu |
| 2-5-259 | N-tolyl | N-tolyl | methyl | methyl | methyl |
| 2-5-260 | N-tolyl | N-tolyl | b1 | H | H |
| 2-5-261 | N-tolyl | N-tolyl | b2 | H | H |
| 2-5-262 | N-tolyl | N-tolyl | b3 | H | H |
| 2-5-263 | N-tolyl | N-tolyl | H | H | a1 |
| 2-5-264 | N-tolyl | N-tolyl | t-bu | H | a1 |
| 2-5-265 | N-tolyl | N-tolyl | methyl | H | a1 |
| 2-5-266 | N-tolyl | N-tolyl | b1 | H | a1 |
| 2-5-267 | N-tolyl | N-tolyl | b2 | H | a1 |
| 2-5-268 | N-tolyl | N-tolyl | b3 | H | a1 |
| 2-5-269 | N-tolyl | N-tolyl | H | H | a2 |
| 2-5-270 | N-tolyl | N-tolyl | t-bu | H | a2 |
| 2-5-271 | N-tolyl | N-tolyl | methyl | H | a2 |
| 2-5-272 | N-tolyl | N-tolyl | b1 | H | a2 |
| 2-5-273 | N-tolyl | N-tolyl | b2 | H | a2 |
| 2-5-274 | N-tolyl | N-tolyl | b3 | H | a2 |
| 2-5-275 | N-tolyl | N-tolyl | H | H | a3 |
| 2-5-276 | N-tolyl | N-tolyl | t-bu | H | a3 |
| 2-5-277 | N-tolyl | N-tolyl | methyl | H | a3 |
| 2-5-278 | N-tolyl | N-tolyl | b1 | H | a3 |
| 2-5-279 | N-tolyl | N-tolyl | b2 | H | a3 |
| 2-5-280 | N-tolyl | N-tolyl | b3 | H | a3 |
| 2-5-281 | N-tolyl | N-tolyl | H | H | a4 |
| 2-5-282 | N-tolyl | N-tolyl | t-bu | H | a4 |
| 2-5-283 | N-tolyl | N-tolyl | methyl | H | a4 |
| 2-5-284 | N-tolyl | N-tolyl | b1 | H | a4 |
| 2-5-285 | N-tolyl | N-tolyl | b2 | H | a4 |
| 2-5-286 | N-tolyl | N-tolyl | b3 | H | a4 |
| 2-5-287 | N-tolyl | N-tolyl | H | H | a5 |
| 2-5-288 | N-tolyl | N-tolyl | t-bu | H | a5 |
| 2-5-289 | N-tolyl | N-tolyl | methyl | H | a5 |
| 2-5-290 | N-tolyl | N-tolyl | b1 | H | a5 |
| 2-5-291 | N-tolyl | N-tolyl | b2 | H | a5 |
| 2-5-292 | N-tolyl | N-tolyl | b3 | H | a5 |
| 2-5-293 | N-tolyl | N-tolyl | H | H | a6 |
| 2-5-294 | N-tolyl | N-tolyl | t-bu | H | a6 |
| 2-5-295 | N-tolyl | N-tolyl | methyl | H | a6 |
| 2-5-296 | N-tolyl | N-tolyl | b1 | H | a6 |
| 2-5-297 | N-tolyl | N-tolyl | b2 | H | a6 |
| 2-5-298 | N-tolyl | N-tolyl | b3 | H | a6 |
| 2-5-299 | N-tolyl | N-tolyl | H | H | b1 |
| 2-5-300 | N-tolyl | N-tolyl | t-bu | H | b1 |
| 2-5-301 | N-tolyl | N-tolyl | methyl | H | b1 |
| 2-5-302 | N-tolyl | N-tolyl | b1 | H | b1 |
| 2-5-303 | N-tolyl | N-tolyl | b2 | H | b1 |
| 2-5-304 | N-tolyl | N-tolyl | b3 | H | b1 |
| 2-5-305 | N-tolyl | N-tolyl | H | H | b2 |
| 2-5-306 | N-tolyl | N-tolyl | t-bu | H | b2 |
| 2-5-307 | N-tolyl | N-tolyl | methyl | H | b2 |
| 2-5-308 | N-tolyl | N-tolyl | b1 | H | b2 |
| 2-5-309 | N-tolyl | N-tolyl | b2 | H | b2 |
| 2-5-310 | N-tolyl | N-tolyl | b3 | H | b2 |
| 2-5-311 | N-tolyl | N-tolyl | H | H | b3 |
| 2-5-312 | N-tolyl | N-tolyl | t-bu | H | b3 |
| 2-5-313 | N-tolyl | N-tolyl | methyl | H | b3 |
| 2-5-314 | N-tolyl | N-tolyl | b1 | H | b3 |
| 2-5-315 | N-tolyl | N-tolyl | b2 | H | b3 |
| 2-5-316 | N-tolyl | N-tolyl | b3 | H | b3 |
| 2-5-317 | N-tolyl | N-tolyl | H | H | b4 |
| 2-5-318 | N-tolyl | N-tolyl | t-bu | H | b4 |
| 2-5-319 | N-tolyl | N-tolyl | methyl | H | b4 |
| 2-5-320 | N-tolyl | N-tolyl | b1 | H | b4 |
| 2-5-321 | N-tolyl | N-tolyl | b2 | H | b4 |
| 2-5-322 | N-tolyl | N-tolyl | b3 | H | b4 |
| 2-5-323 | N-tolyl | N-tolyl | H | H | b5 |
| 2-5-324 | N-tolyl | N-tolyl | t-bu | H | b5 |
| 2-5-325 | N-tolyl | N-tolyl | methyl | H | b5 |
| 2-5-326 | N-tolyl | N-tolyl | b1 | H | b5 |
| 2-5-327 | N-tolyl | N-tolyl | b2 | H | b5 |
| 2-5-328 | N-tolyl | N-tolyl | b3 | H | b5 |
| 2-5-329 | N-tolyl | N-tolyl | H | H | b6 |
| 2-5-330 | N-tolyl | N-tolyl | t-bu | H | b6 |
| 2-5-331 | N-tolyl | N-tolyl | methyl | H | b6 |
| 2-5-332 | N-tolyl | N-tolyl | b1 | H | b6 |
| 2-5-333 | N-tolyl | N-tolyl | b2 | H | b6 |
| 2-5-334 | N-tolyl | N-tolyl | b3 | H | b6 |
| 2-5-335 | N-tolyl | N-tolyl | H | H | b7 |
| 2-5-336 | N-tolyl | N-tolyl | t-bu | H | b7 |
| 2-5-337 | N-tolyl | N-tolyl | methyl | H | b7 |
| 2-5-338 | N-tolyl | N-tolyl | b1 | H | b7 |
| 2-5-339 | N-tolyl | N-tolyl | b2 | H | b7 |
| 2-5-340 | N-tolyl | N-tolyl | b3 | H | b7 |
| 2-5-341 | N-tolyl | N-tolyl | H | H | b8 |
| 2-5-342 | N-tolyl | N-tolyl | t-bu | H | b8 |
| 2-5-343 | N-tolyl | N-tolyl | methyl | H | b8 |
| 2-5-344 | N-tolyl | N-tolyl | b1 | H | b8 |
| 2-5-345 | N-tolyl | N-tolyl | b2 | H | b8 |
| 2-5-346 | N-tolyl | N-tolyl | b3 | H | b8 |
| 2-5-347 | N-tolyl | N-tolyl | H | H | b9 |
| 2-5-348 | N-tolyl | N-tolyl | t-bu | H | b9 |
| 2-5-349 | N-tolyl | N-tolyl | methyl | H | b9 |
| 2-5-350 | N-tolyl | N-tolyl | b1 | H | b9 |
| 2-5-351 | N-tolyl | N-tolyl | b2 | H | b9 |
| 2-5-352 | N-tolyl | N-tolyl | b3 | H | b9 |
| 2-5-353 | N-tolyl | N-tolyl | H | H | b10 |
| 2-5-354 | N-tolyl | N-tolyl | t-bu | H | b10 |
| 2-5-355 | N-tolyl | N-tolyl | methyl | H | b10 |
| 2-5-356 | N-tolyl | N-tolyl | b1 | H | b10 |
| 2-5-357 | N-tolyl | N-tolyl | b2 | H | b10 |
| 2-5-358 | N-tolyl | N-tolyl | b3 | H | b10 |
| 2-5-359 | N-tolyl | N-tolyl | H | a1 | H |
| 2-5-360 | N-tolyl | N-tolyl | t-bu | a1 | H |
| 2-5-361 | N-tolyl | N-tolyl | methyl | a1 | H |
| 2-5-362 | N-tolyl | N-tolyl | b1 | a1 | H |
| 2-5-363 | N-tolyl | N-tolyl | b2 | a1 | H |
| 2-5-364 | N-tolyl | N-tolyl | b3 | a1 | H |
| 2-5-365 | N-tolyl | N-tolyl | H | a2 | H |
| 2-5-366 | N-tolyl | N-tolyl | t-bu | a2 | H |
| 2-5-367 | N-tolyl | N-tolyl | methyl | a2 | H |
| 2-5-368 | N-tolyl | N-tolyl | b1 | a2 | H |
| 2-5-369 | N-tolyl | N-tolyl | b2 | a2 | H |
| 2-5-370 | N-tolyl | N-tolyl | b3 | a2 | H |
| 2-5-371 | N-tolyl | N-tolyl | H | a3 | H |
| 2-5-372 | N-tolyl | N-tolyl | t-bu | a3 | H |
| 2-5-373 | N-tolyl | N-tolyl | methyl | a3 | H |
| 2-5-374 | N-tolyl | N-tolyl | b1 | a3 | H |
| 2-5-375 | N-tolyl | N-tolyl | b2 | a3 | H |
| 2-5-376 | N-tolyl | N-tolyl | b3 | a3 | H |
| 2-5-377 | N-tolyl | N-tolyl | H | a4 | H |
| 2-5-378 | N-tolyl | N-tolyl | t-bu | a4 | H |
| 2-5-379 | N-tolyl | N-tolyl | methyl | a4 | H |
| 2-5-380 | N-tolyl | N-tolyl | b1 | a4 | H |
| 2-5-381 | N-tolyl | N-tolyl | b2 | a4 | H |
| 2-5-382 | N-tolyl | N-tolyl | b3 | a4 | H |
| 2-5-383 | N-tolyl | N-tolyl | H | a5 | H |
| 2-5-384 | N-tolyl | N-tolyl | t-bu | a5 | H |
| 2-5-385 | N-tolyl | N-tolyl | methyl | a5 | H |
| 2-5-386 | N-tolyl | N-tolyl | b1 | a5 | H |
| 2-5-387 | N-tolyl | N-tolyl | b2 | a5 | H |
| 2-5-388 | N-tolyl | N-tolyl | b3 | a5 | H |
| 2-5-389 | N-tolyl | N-tolyl | H | a6 | H |
| 2-5-390 | N-tolyl | N-tolyl | t-bu | a6 | H |
| 2-5-391 | N-tolyl | N-tolyl | methyl | a6 | H |
| 2-5-392 | N-tolyl | N-tolyl | b1 | a6 | H |
| 2-5-393 | N-tolyl | N-tolyl | b2 | a6 | H |
| 2-5-394 | N-tolyl | N-tolyl | b3 | a6 | H |
| 2-5-395 | N-tolyl | N-tolyl | H | b1 | H |
| 2-5-396 | N-tolyl | N-tolyl | t-bu | b1 | H |
| 2-5-397 | N-tolyl | N-tolyl | methyl | b1 | H |
| 2-5-398 | N-tolyl | N-tolyl | b1 | b1 | H |
| 2-5-399 | N-tolyl | N-tolyl | b2 | b1 | H |
| 2-5-400 | N-tolyl | N-tolyl | b3 | b1 | H |
| 2-5-401 | N-tolyl | N-tolyl | H | b2 | H |
| 2-5-402 | N-tolyl | N-tolyl | t-bu | b2 | H |
| 2-5-403 | N-tolyl | N-tolyl | methyl | b2 | H |
| 2-5-404 | N-tolyl | N-tolyl | b1 | b2 | H |
| 2-5-405 | N-tolyl | N-tolyl | b2 | b2 | H |
| 2-5-406 | N-tolyl | N-tolyl | b3 | b2 | H |
| 2-5-407 | N-tolyl | N-tolyl | H | b3 | H |
| 2-5-408 | N-tolyl | N-tolyl | t-bu | b3 | H |
| 2-5-409 | N-tolyl | N-tolyl | methyl | b3 | H |
| 2-5-410 | N-tolyl | N-tolyl | b1 | b3 | H |
| 2-5-411 | N-tolyl | N-tolyl | b2 | b3 | H |
| 2-5-412 | N-tolyl | N-tolyl | b3 | b3 | H |
| 2-5-413 | N-tolyl | N-tolyl | H | b4 | H |
| 2-5-414 | N-tolyl | N-tolyl | t-bu | b4 | H |
| 2-5-415 | N-tolyl | N-tolyl | methyl | b4 | H |
| 2-5-416 | N-tolyl | N-tolyl | b1 | b4 | H |
| 2-5-417 | N-tolyl | N-tolyl | b2 | b4 | H |
| 2-5-418 | N-tolyl | N-tolyl | b3 | b4 | H |
| 2-5-419 | N-tolyl | N-tolyl | H | b5 | H |
| 2-5-420 | N-tolyl | N-tolyl | t-bu | b5 | H |
| 2-5-421 | N-tolyl | N-tolyl | methyl | b5 | H |
| 2-5-422 | N-tolyl | N-tolyl | b1 | b5 | H |
| 2-5-423 | N-tolyl | N-tolyl | b2 | b5 | H |
| 2-5-424 | N-tolyl | N-tolyl | b3 | b5 | H |
| 2-5-425 | N-tolyl | N-tolyl | H | b6 | H |
| 2-5-426 | N-tolyl | N-tolyl | t-bu | b6 | H |
| 2-5-427 | N-tolyl | N-tolyl | methyl | b6 | H |
| 2-5-428 | N-tolyl | N-tolyl | b1 | b6 | H |
| 2-5-429 | N-tolyl | N-tolyl | b2 | b6 | H |
| 2-5-430 | N-tolyl | N-tolyl | b3 | b6 | H |
| 2-5-431 | N-tolyl | N-tolyl | H | b7 | H |
| 2-5-432 | N-tolyl | N-tolyl | t-bu | b7 | H |
| 2-5-433 | N-tolyl | N-tolyl | methyl | b7 | H |
| 2-5-434 | N-tolyl | N-tolyl | b1 | b7 | H |
| 2-5-435 | N-tolyl | N-tolyl | b2 | b7 | H |
| 2-5-436 | N-tolyl | N-tolyl | b3 | b7 | H |
| 2-5-437 | N-tolyl | N-tolyl | H | b8 | H |
| 2-5-438 | N-tolyl | N-tolyl | t-bu | b8 | H |
| 2-5-439 | N-tolyl | N-tolyl | methyl | b8 | H |
| 2-5-440 | N-tolyl | N-tolyl | b1 | b8 | H |
| 2-5-441 | N-tolyl | N-tolyl | b2 | b8 | H |
| 2-5-442 | N-tolyl | N-tolyl | b3 | b8 | H |
| 2-5-443 | N-tolyl | N-tolyl | H | b9 | H |
| 2-5-444 | N-tolyl | N-tolyl | t-bu | b9 | H |
| 2-5-445 | N-tolyl | N-tolyl | methyl | b9 | H |
| 2-5-446 | N-tolyl | N-tolyl | b1 | b9 | H |
| 2-5-447 | N-tolyl | N-tolyl | b2 | b9 | H |
| 2-5-448 | N-tolyl | N-tolyl | b3 | b9 | H |
| 2-5-449 | N-tolyl | N-tolyl | H | b10 | H |
| 2-5-450 | N-tolyl | N-tolyl | t-bu | b10 | H |
| 2-5-451 | N-tolyl | N-tolyl | methyl | b10 | H |
| 2-5-452 | N-tolyl | N-tolyl | b1 | b10 | H |
| 2-5-453 | N-tolyl | N-tolyl | b2 | b10 | H |
| 2-5-454 | N-tolyl | N-tolyl | b3 | b10 | H |
| 2-5-455 | N-tolyl | N-tolyl | H | a1 | a1 |
| 2-5-456 | N-tolyl | N-tolyl | t-bu | a1 | a1 |
| 2-5-457 | N-tolyl | N-tolyl | methyl | a1 | a1 |
| 2-5-458 | N-tolyl | N-tolyl | b1 | a1 | a1 |
| 2-5-459 | N-tolyl | N-tolyl | b2 | a1 | a1 |
| 2-5-460 | N-tolyl | N-tolyl | b3 | a1 | a1 |
| 2-5-461 | N-tolyl | N-tolyl | H | a2 | a2 |
| 2-5-462 | N-tolyl | N-tolyl | t-bu | a2 | a2 |
| 2-5-463 | N-tolyl | N-tolyl | methyl | a2 | a2 |
| 2-5-464 | N-tolyl | N-tolyl | b1 | a2 | a2 |
| 2-5-465 | N-tolyl | N-tolyl | b2 | a2 | a2 |
| 2-5-466 | N-tolyl | N-tolyl | b3 | a2 | a2 |
| 2-5-467 | N-tolyl | N-tolyl | H | a3 | a3 |
| 2-5-468 | N-tolyl | N-tolyl | t-bu | a3 | a3 |
| 2-5-469 | N-tolyl | N-tolyl | methyl | a3 | a3 |
| 2-5-470 | N-tolyl | N-tolyl | b1 | a3 | a3 |
| 2-5-471 | N-tolyl | N-tolyl | b2 | a3 | a3 |
| 2-5-472 | N-tolyl | N-tolyl | b3 | a3 | a3 |
| 2-5-473 | N-tolyl | N-tolyl | H | b1 | b1 |
| 2-5-474 | N-tolyl | N-tolyl | t-bu | b1 | b1 |
| 2-5-475 | N-tolyl | N-tolyl | methyl | b1 | b1 |
| 2-5-476 | N-tolyl | N-tolyl | b1 | b1 | b1 |
| 2-5-477 | N-tolyl | N-tolyl | b2 | b1 | b1 |
| 2-5-478 | N-tolyl | N-tolyl | b3 | b1 | b1 |
| 2-5-479 | N-tolyl | N-tolyl | H | b2 | b2 |
| 2-5-480 | N-tolyl | N-tolyl | t-bu | b2 | b2 |
| 2-5-481 | N-tolyl | N-tolyl | methyl | b2 | b2 |
| 2-5-482 | N-tolyl | N-tolyl | b1 | b2 | b2 |
| 2-5-483 | N-tolyl | N-tolyl | b2 | b2 | b2 |
| 2-5-484 | N-tolyl | N-tolyl | b3 | b2 | b2 |
| 2-5-485 | N-tolyl | N-tolyl | H | b3 | b3 |
| 2-5-486 | N-tolyl | N-tolyl | t-bu | b3 | b3 |
| 2-5-487 | N-tolyl | N-tolyl | methyl | b3 | b3 |
| 2-5-488 | N-tolyl | N-tolyl | b1 | b3 | b3 |
| 2-5-489 | N-tolyl | N-tolyl | b2 | b3 | b3 |
| 2-5-490 | N-tolyl | N-tolyl | b3 | b3 | b3 |
| 2-5-491 | N-tolyl | N-tolyl | H | b5 | b5 |
| 2-5-492 | N-tolyl | N-tolyl | t-bu | b5 | b5 |
| 2-5-493 | N-tolyl | N-tolyl | methyl | b5 | b5 |
| 2-5-494 | N-tolyl | N-tolyl | b1 | b5 | b5 |
| 2-5-495 | N-tolyl | N-tolyl | b2 | b5 | b5 |
| 2-5-496 | N-tolyl | N-tolyl | b3 | b5 | b5 |
| 2-5-497 | N-tolyl | N-tolyl | H | b6 | b6 |
| 2-5-498 | N-tolyl | N-tolyl | t-bu | b6 | b6 |
| 2-5-499 | N-tolyl | N-tolyl | methyl | b6 | b6 |
| 2-5-500 | N-tolyl | N-tolyl | b1 | b6 | b6 |
| 2-5-501 | N-tolyl | N-tolyl | b2 | b6 | b6 |
| 2-5-502 | N-tolyl | N-tolyl | b3 | b6 | b6 |
| 2-5-503 | N-tolyl | N-tolyl | H | b7 | b7 |
| 2-5-504 | N-tolyl | N-tolyl | t-bu | b7 | b7 |
| 2-5-505 | N-tolyl | N-tolyl | methyl | b7 | b7 |
| 2-5-506 | N-tolyl | N-tolyl | b1 | b7 | b7 |
| 2-5-507 | N-tolyl | N-tolyl | b2 | b7 | b7 |
| 2-5-508 | N-tolyl | N-tolyl | b3 | b7 | b7 |
| 2-5-509 | N-Biph | N-Biph | H | H | H |
| 2-5-510 | N-Biph | N-Biph | t-bu | H | H |
| 2-5-511 | N-Biph | N-Biph | methyl | H | H |
| 2-5-512 | N-Biph | N-Biph | t-bu | t-bu | t-bu |
| 2-5-513 | N-Biph | N-Biph | methyl | methyl | methyl |
| 2-5-514 | N-Biph | N-Biph | b1 | H | H |
| 2-5-515 | N-Biph | N-Biph | b2 | H | H |
| 2-5-516 | N-Biph | N-Biph | b3 | H | H |
| 2-5-517 | N-Biph | N-Biph | H | H | a1 |
| 2-5-518 | N-Biph | N-Biph | t-bu | H | a1 |
| 2-5-519 | N-Biph | N-Biph | methyl | H | a1 |
| 2-5-520 | N-Biph | N-Biph | b1 | H | a1 |
| 2-5-521 | N-Biph | N-Biph | b2 | H | a1 |
| 2-5-522 | N-Biph | N-Biph | b3 | H | a1 |
| 2-5-523 | N-Biph | N-Biph | H | H | a2 |
| 2-5-524 | N-Biph | N-Biph | t-bu | H | a2 |
| 2-5-525 | N-Biph | N-Biph | methyl | H | a2 |
| 2-5-526 | N-Biph | N-Biph | b1 | H | a2 |
| 2-5-527 | N-Biph | N-Biph | b2 | H | a2 |
| 2-5-528 | N-Biph | N-Biph | b3 | H | a2 |
| 2-5-529 | N-Biph | N-Biph | H | H | a3 |
| 2-5-530 | N-Biph | N-Biph | t-bu | H | a3 |
| 2-5-531 | N-Biph | N-Biph | methyl | H | a3 |
| 2-5-532 | N-Biph | N-Biph | b1 | H | a3 |
| 2-5-533 | N-Biph | N-Biph | b2 | H | a3 |
| 2-5-534 | N-Biph | N-Biph | b3 | H | a3 |
| 2-5-535 | N-Biph | N-Biph | H | H | a4 |
| 2-5-536 | N-Biph | N-Biph | t-bu | H | a4 |
| 2-5-537 | N-Biph | N-Biph | methyl | H | a4 |
| 2-5-538 | N-Biph | N-Biph | b1 | H | a4 |
| 2-5-539 | N-Biph | N-Biph | b2 | H | a4 |
| 2-5-540 | N-Biph | N-Biph | b3 | H | a4 |
| 2-5-541 | N-Biph | N-Biph | H | H | a5 |
| 2-5-542 | N-Biph | N-Biph | t-bu | H | a5 |
| 2-5-543 | N-Biph | N-Biph | methyl | H | a5 |
| 2-5-544 | N-Biph | N-Biph | b1 | H | a5 |
| 2-5-545 | N-Biph | N-Biph | b2 | H | a5 |
| 2-5-546 | N-Biph | N-Biph | b3 | H | a5 |
| 2-5-547 | N-Biph | N-Biph | H | H | a6 |
| 2-5-548 | N-Biph | N-Biph | t-bu | H | a6 |
| 2-5-549 | N-Biph | N-Biph | methyl | H | a6 |
| 2-5-550 | N-Biph | N-Biph | b1 | H | a6 |
| 2-5-551 | N-Biph | N-Biph | b2 | H | a6 |
| 2-5-552 | N-Biph | N-Biph | b3 | H | a6 |
| 2-5-553 | N-Biph | N-Biph | H | H | b1 |
| 2-5-554 | N-Biph | N-Biph | t-bu | H | b1 |
| 2-5-555 | N-Biph | N-Biph | methyl | H | b1 |
| 2-5-556 | N-Biph | N-Biph | b1 | H | b1 |
| 2-5-557 | N-Biph | N-Biph | b2 | H | b1 |
| 2-5-558 | N-Biph | N-Biph | b3 | H | b1 |
| 2-5-559 | N-Biph | N-Biph | H | H | b2 |
| 2-5-560 | N-Biph | N-Biph | t-bu | H | b2 |
| 2-5-561 | N-Biph | N-Biph | methyl | H | b2 |
| 2-5-562 | N-Biph | N-Biph | b1 | H | b2 |
| 2-5-563 | N-Biph | N-Biph | b2 | H | b2 |
| 2-5-564 | N-Biph | N-Biph | b3 | H | b2 |
| 2-5-565 | N-Biph | N-Biph | H | H | b3 |
| 2-5-566 | N-Biph | N-Biph | t-bu | H | b3 |
| 2-5-567 | N-Biph | N-Biph | methyl | H | b3 |
| 2-5-568 | N-Biph | N-Biph | b1 | H | b3 |
| 2-5-569 | N-Biph | N-Biph | b2 | H | b3 |
| 2-5-570 | N-Biph | N-Biph | b3 | H | b3 |
| 2-5-571 | N-Biph | N-Biph | H | H | b4 |
| 2-5-572 | N-Biph | N-Biph | t-bu | H | b4 |
| 2-5-573 | N-Biph | N-Biph | methyl | H | b4 |
| 2-5-574 | N-Biph | N-Biph | b1 | H | b4 |
| 2-5-575 | N-Biph | N-Biph | b2 | H | b4 |
| 2-5-576 | N-Biph | N-Biph | b3 | H | b4 |
| 2-5-577 | N-Biph | N-Biph | H | H | b5 |
| 2-5-578 | N-Biph | N-Biph | t-bu | H | b5 |
| 2-5-579 | N-Biph | N-Biph | methyl | H | b5 |
| 2-5-580 | N-Biph | N-Biph | b1 | H | b5 |
| 2-5-581 | N-Biph | N-Biph | b2 | H | b5 |
| 2-5-582 | N-Biph | N-Biph | b3 | H | b5 |
| 2-5-583 | N-Biph | N-Biph | H | H | b6 |
| 2-5-584 | N-Biph | N-Biph | t-bu | H | b6 |
| 2-5-585 | N-Biph | N-Biph | methyl | H | b6 |
| 2-5-586 | N-Biph | N-Biph | b1 | H | b6 |
| 2-5-587 | N-Biph | N-Biph | b2 | H | b6 |
| 2-5-588 | N-Biph | N-Biph | b3 | H | b6 |
| 2-5-589 | N-Biph | N-Biph | H | H | b7 |
| 2-5-590 | N-Biph | N-Biph | t-bu | H | b7 |
| 2-5-591 | N-Biph | N-Biph | methyl | H | b7 |
| 2-5-592 | N-Biph | N-Biph | b1 | H | b7 |
| 2-5-593 | N-Biph | N-Biph | b2 | H | b7 |
| 2-5-594 | N-Biph | N-Biph | b3 | H | b7 |
| 2-5-595 | N-Biph | N-Biph | H | H | b8 |
| 2-5-596 | N-Biph | N-Biph | t-bu | H | b8 |
| 2-5-597 | N-Biph | N-Biph | methyl | H | b8 |
| 2-5-598 | N-Biph | N-Biph | b1 | H | b8 |
| 2-5-599 | N-Biph | N-Biph | b2 | H | b8 |
| 2-5-600 | N-Biph | N-Biph | b3 | H | b8 |
| 2-5-601 | N-Biph | N-Biph | H | H | b9 |
| 2-5-602 | N-Biph | N-Biph | t-bu | H | b9 |
| 2-5-603 | N-Biph | N-Biph | methyl | H | b9 |
| 2-5-604 | N-Biph | N-Biph | b1 | H | b9 |
| 2-5-605 | N-Biph | N-Biph | b2 | H | b9 |
| 2-5-606 | N-Biph | N-Biph | b3 | H | b9 |
| 2-5-607 | N-Biph | N-Biph | H | H | b10 |
| 2-5-608 | N-Biph | N-Biph | t-bu | H | b10 |
| 2-5-609 | N-Biph | N-Biph | methyl | H | b10 |
| 2-5-610 | N-Biph | N-Biph | b1 | H | b10 |
| 2-5-611 | N-Biph | N-Biph | b2 | H | b10 |
| 2-5-612 | N-Biph | N-Biph | b3 | H | b10 |
| 2-5-613 | N-Biph | N-Biph | H | a1 | H |
| 2-5-614 | N-Biph | N-Biph | t-bu | a1 | H |
| 2-5-615 | N-Biph | N-Biph | methyl | a1 | H |
| 2-5-616 | N-Biph | N-Biph | b1 | a1 | H |
| 2-5-617 | N-Biph | N-Biph | b2 | a1 | H |
| 2-5-618 | N-Biph | N-Biph | b3 | a1 | H |
| 2-5-619 | N-Biph | N-Biph | H | a2 | H |
| 2-5-620 | N-Biph | N-Biph | t-bu | a2 | H |
| 2-5-621 | N-Biph | N-Biph | methyl | a2 | H |
| 2-5-622 | N-Biph | N-Biph | b1 | a2 | H |
| 2-5-623 | N-Biph | N-Biph | b2 | a2 | H |
| 2-5-624 | N-Biph | N-Biph | b3 | a2 | H |
| 2-5-625 | N-Biph | N-Biph | H | a3 | H |
| 2-5-626 | N-Biph | N-Biph | t-bu | a3 | H |
| 2-5-627 | N-Biph | N-Biph | methyl | a3 | H |
| 2-5-628 | N-Biph | N-Biph | b1 | a3 | H |
| 2-5-629 | N-Biph | N-Biph | b2 | a3 | H |
| 2-5-630 | N-Biph | N-Biph | b3 | a3 | H |
| 2-5-631 | N-Biph | N-Biph | H | a4 | H |
| 2-5-632 | N-Biph | N-Biph | t-bu | a4 | H |
| 2-5-633 | N-Biph | N-Biph | methyl | a4 | H |
| 2-5-634 | N-Biph | N-Biph | b1 | a4 | H |
| 2-5-635 | N-Biph | N-Biph | b2 | a4 | H |
| 2-5-636 | N-Biph | N-Biph | b3 | a4 | H |
| 2-5-637 | N-Biph | N-Biph | H | a5 | H |
| 2-5-638 | N-Biph | N-Biph | t-bu | a5 | H |
| 2-5-639 | N-Biph | N-Biph | methyl | a5 | H |
| 2-5-640 | N-Biph | N-Biph | b1 | a5 | H |
| 2-5-641 | N-Biph | N-Biph | b2 | a5 | H |
| 2-5-642 | N-Biph | N-Biph | b3 | a5 | H |
| 2-5-643 | N-Biph | N-Biph | H | a6 | H |
| 2-5-644 | N-Biph | N-Biph | t-bu | a6 | H |
| 2-5-645 | N-Biph | N-Biph | methyl | a6 | H |
| 2-5-646 | N-Biph | N-Biph | b1 | a6 | H |
| 2-5-647 | N-Biph | N-Biph | b2 | a6 | H |
| 2-5-648 | N-Biph | N-Biph | b3 | a6 | H |
| 2-5-649 | N-Biph | N-Biph | H | b1 | H |
| 2-5-650 | N-Biph | N-Biph | t-bu | b1 | H |
| 2-5-651 | N-Biph | N-Biph | methyl | b1 | H |
| 2-5-652 | N-Biph | N-Biph | b1 | b1 | H |
| 2-5-653 | N-Biph | N-Biph | b2 | b1 | H |
| 2-5-654 | N-Biph | N-Biph | b3 | b1 | H |
| 2-5-655 | N-Biph | N-Biph | H | b2 | H |
| 2-5-656 | N-Biph | N-Biph | t-bu | b2 | H |
| 2-5-657 | N-Biph | N-Biph | methyl | b2 | H |
| 2-5-658 | N-Biph | N-Biph | b1 | b2 | H |
| 2-5-659 | N-Biph | N-Biph | b2 | b2 | H |
| 2-5-660 | N-Biph | N-Biph | b3 | b2 | H |
| 2-5-661 | N-Biph | N-Biph | H | b3 | H |
| 2-5-662 | N-Biph | N-Biph | t-bu | b3 | H |
| 2-5-663 | N-Biph | N-Biph | methyl | b3 | H |
| 2-5-664 | N-Biph | N-Biph | b1 | b3 | H |
| 2-5-665 | N-Biph | N-Biph | b2 | b3 | H |
| 2-5-666 | N-Biph | N-Biph | b3 | b3 | H |
| 2-5-667 | N-Biph | N-Biph | H | b4 | H |
| 2-5-668 | N-Biph | N-Biph | t-bu | b4 | H |
| 2-5-669 | N-Biph | N-Biph | methyl | b4 | H |
| 2-5-670 | N-Biph | N-Biph | b1 | b4 | H |
| 2-5-671 | N-Biph | N-Biph | b2 | b4 | H |
| 2-5-672 | N-Biph | N-Biph | b3 | b4 | H |
| 2-5-673 | N-Biph | N-Biph | H | b5 | H |
| 2-5-674 | N-Biph | N-Biph | t-bu | b5 | H |
| 2-5-675 | N-Biph | N-Biph | methyl | b5 | H |
| 2-5-676 | N-Biph | N-Biph | b1 | b5 | H |
| 2-5-677 | N-Biph | N-Biph | b2 | b5 | H |
| 2-5-678 | N-Biph | N-Biph | b3 | b5 | H |
| 2-5-679 | N-Biph | N-Biph | H | b6 | H |
| 2-5-680 | N-Biph | N-Biph | t-bu | b6 | H |
| 2-5-681 | N-Biph | N-Biph | methyl | b6 | H |
| 2-5-682 | N-Biph | N-Biph | b1 | b6 | H |
| 2-5-683 | N-Biph | N-Biph | b2 | b6 | H |
| 2-5-684 | N-Biph | N-Biph | b3 | b6 | H |
| 2-5-685 | N-Biph | N-Biph | H | b7 | H |
| 2-5-686 | N-Biph | N-Biph | t-bu | b7 | H |
| 2-5-687 | N-Biph | N-Biph | methyl | b7 | H |
| 2-5-688 | N-Biph | N-Biph | b1 | b7 | H |
| 2-5-689 | N-Biph | N-Biph | b2 | b7 | H |
| 2-5-690 | N-Biph | N-Biph | b3 | b7 | H |
| 2-5-691 | N-Biph | N-Biph | H | b8 | H |
| 2-5-692 | N-Biph | N-Biph | t-bu | b8 | H |
| 2-5-693 | N-Biph | N-Biph | methyl | b8 | H |
| 2-5-694 | N-Biph | N-Biph | b1 | b8 | H |
| 2-5-695 | N-Biph | N-Biph | b2 | b8 | H |
| 2-5-696 | N-Biph | N-Biph | b3 | b8 | H |
| 2-5-697 | N-Biph | N-Biph | H | b9 | H |
| 2-5-698 | N-Biph | N-Biph | t-bu | b9 | H |
| 2-5-699 | N-Biph | N-Biph | methyl | b9 | H |
| 2-5-700 | N-Biph | N-Biph | b1 | b9 | H |
| 2-5-701 | N-Biph | N-Biph | b2 | b9 | H |
| 2-5-702 | N-Biph | N-Biph | b3 | b9 | H |
| 2-5-703 | N-Biph | N-Biph | H | b10 | H |
| 2-5-704 | N-Biph | N-Biph | t-bu | b10 | H |
| 2-5-705 | N-Biph | N-Biph | methyl | b10 | H |
| 2-5-706 | N-Biph | N-Biph | b1 | b10 | H |
| 2-5-707 | N-Biph | N-Biph | b2 | b10 | H |
| 2-5-708 | N-Biph | N-Biph | b3 | b10 | H |
| 2-5-709 | N-Biph | N-Biph | H | a1 | a1 |
| 2-5-710 | N-Biph | N-Biph | t-bu | a1 | a1 |
| 2-5-711 | N-Biph | N-Biph | methyl | a1 | a1 |
| 2-5-712 | N-Biph | N-Biph | b1 | a1 | a1 |
| 2-5-713 | N-Biph | N-Biph | b2 | a1 | a1 |
| 2-5-714 | N-Biph | N-Biph | b3 | a1 | a1 |
| 2-5-715 | N-Biph | N-Biph | H | a2 | a2 |
| 2-5-716 | N-Biph | N-Biph | t-bu | a2 | a2 |
| 2-5-717 | N-Biph | N-Biph | methyl | a2 | a2 |
| 2-5-718 | N-Biph | N-Biph | b1 | a2 | a2 |
| 2-5-719 | N-Biph | N-Biph | b2 | a2 | a2 |
| 2-5-720 | N-Biph | N-Biph | b3 | a2 | a2 |
| 2-5-721 | N-Biph | N-Biph | H | a3 | a3 |
| 2-5-722 | N-Biph | N-Biph | t-bu | a3 | a3 |
| 2-5-723 | N-Biph | N-Biph | methyl | a3 | a3 |
| 2-5-724 | N-Biph | N-Biph | b1 | a3 | a3 |
| 2-5-725 | N-Biph | N-Biph | b2 | a3 | a3 |
| 2-5-726 | N-Biph | N-Biph | b3 | a3 | a3 |
| 2-5-727 | N-Biph | N-Biph | H | b1 | b1 |
| 2-5-728 | N-Biph | N-Biph | t-bu | b1 | b1 |
| 2-5-729 | N-Biph | N-Biph | methyl | b1 | b1 |
| 2-5-730 | N-Biph | N-Biph | b1 | b1 | b1 |
| 2-5-731 | N-Biph | N-Biph | b2 | b1 | b1 |
| 2-5-732 | N-Biph | N-Biph | b3 | b1 | b1 |
| 2-5-733 | N-Biph | N-Biph | H | b2 | b2 |
| 2-5-734 | N-Biph | N-Biph | t-bu | b2 | b2 |
| 2-5-735 | N-Biph | N-Biph | methyl | b2 | b2 |
| 2-5-736 | N-Biph | N-Biph | b1 | b2 | b2 |
| 2-5-737 | N-Biph | N-Biph | b2 | b2 | b2 |
| 2-5-738 | N-Biph | N-Biph | b3 | b2 | b2 |
| 2-5-739 | N-Biph | N-Biph | H | b3 | b3 |
| 2-5-740 | N-Biph | N-Biph | t-bu | b3 | b3 |
| 2-5-741 | N-Biph | N-Biph | methyl | b3 | b3 |
| 2-5-742 | N-Biph | N-Biph | b1 | b3 | b3 |
| 2-5-743 | N-Biph | N-Biph | b2 | b3 | b3 |
| 2-5-744 | N-Biph | N-Biph | b3 | b3 | b3 |
| 2-5-745 | N-Biph | N-Biph | H | b5 | b5 |
| 2-5-746 | N-Biph | N-Biph | t-bu | b5 | b5 |
| 2-5-747 | N-Biph | N-Biph | methyl | b5 | b5 |
| 2-5-748 | N-Biph | N-Biph | b1 | b5 | b5 |
| 2-5-749 | N-Biph | N-Biph | b2 | b5 | b5 |
| 2-5-750 | N-Biph | N-Biph | b3 | b5 | b5 |
| 2-5-751 | N-Biph | N-Biph | H | b6 | b6 |
| 2-5-752 | N-Biph | N-Biph | t-bu | b6 | b6 |
| 2-5-753 | N-Biph | N-Biph | methyl | b6 | b6 |
| 2-5-754 | N-Biph | N-Biph | b1 | b6 | b6 |
| 2-5-755 | N-Biph | N-Biph | b2 | b6 | b6 |
| 2-5-756 | N-Biph | N-Biph | b3 | b6 | b6 |
| 2-5-757 | N-Biph | N-Biph | H | b7 | b7 |
| 2-5-758 | N-Biph | N-Biph | t-bu | b7 | b7 |
| 2-5-759 | N-Biph | N-Biph | methyl | b7 | b7 |
| 2-5-760 | N-Biph | N-Biph | b1 | b7 | b7 |
| 2-5-761 | N-Biph | N-Biph | b2 | b7 | b7 |
| 2-5-762 | N-Biph | N-Biph | b3 | b7 | b7 |
| 2-5-763 | O | N—Ph | H | H | H |
| 2-5-764 | O | N—Ph | t-bu | H | H |
| 2-5-765 | O | N—Ph | methyl | H | H |
| 2-5-766 | O | N—Ph | t-bu | t-bu | t-bu |
| 2-5-767 | O | N—Ph | methyl | methyl | methyl |
| 2-5-768 | O | N—Ph | b1 | H | H |
| 2-5-769 | O | N—Ph | b2 | H | H |
| 2-5-770 | O | N—Ph | b3 | H | H |
| 2-5-771 | O | N—Ph | H | H | a1 |
| 2-5-772 | O | N—Ph | t-bu | H | a1 |
| 2-5-773 | O | N—Ph | methyl | H | a1 |
| 2-5-774 | O | N—Ph | b1 | H | a1 |
| 2-5-775 | O | N—Ph | b2 | H | a1 |
| 2-5-776 | O | N—Ph | b3 | H | a1 |
| 2-5-777 | O | N—Ph | H | H | a2 |
| 2-5-778 | O | N—Ph | t-bu | H | a2 |
| 2-5-779 | O | N—Ph | methyl | H | a2 |
| 2-5-780 | O | N—Ph | b1 | H | a2 |
| 2-5-781 | O | N—Ph | b2 | H | a2 |
| 2-5-782 | O | N—Ph | b3 | H | a2 |
| 2-5-783 | O | N—Ph | H | H | a3 |
| 2-5-784 | O | N—Ph | t-bu | H | a3 |
| 2-5-785 | O | N—Ph | methyl | H | a3 |
| 2-5-786 | O | N—Ph | b1 | H | a3 |
| 2-5-787 | O | N—Ph | b2 | H | a3 |
| 2-5-788 | O | N—Ph | b3 | H | a3 |
| 2-5-789 | O | N—Ph | H | H | a4 |
| 2-5-790 | O | N—Ph | t-bu | H | a4 |
| 2-5-791 | O | N—Ph | methyl | H | a4 |
| 2-5-792 | O | N—Ph | b1 | H | a4 |
| 2-5-793 | O | N—Ph | b2 | H | a4 |
| 2-5-794 | O | N—Ph | b3 | H | a4 |
| 2-5-795 | O | N—Ph | H | H | a5 |
| 2-5-796 | O | N—Ph | t-bu | H | a5 |
| 2-5-797 | O | N—Ph | methyl | H | a5 |
| 2-5-798 | O | N—Ph | b1 | H | a5 |
| 2-5-799 | O | N—Ph | b2 | H | a5 |
| 2-5-800 | O | N—Ph | b3 | H | a5 |
| 2-5-801 | O | N—Ph | H | H | a6 |
| 2-5-802 | O | N—Ph | t-bu | H | a6 |
| 2-5-803 | O | N—Ph | methyl | H | a6 |
| 2-5-804 | O | N—Ph | b1 | H | a6 |
| 2-5-805 | O | N—Ph | b2 | H | a6 |
| 2-5-806 | O | N—Ph | b3 | H | a6 |
| 2-5-807 | O | N—Ph | H | H | b1 |
| 2-5-808 | O | N—Ph | t-bu | H | b1 |
| 2-5-809 | O | N—Ph | methyl | H | b1 |
| 2-5-810 | O | N—Ph | b1 | H | b1 |
| 2-5-811 | O | N—Ph | b2 | H | b1 |
| 2-5-812 | O | N—Ph | b3 | H | b1 |
| 2-5-813 | O | N—Ph | H | H | b2 |
| 2-5-814 | O | N—Ph | t-bu | H | b2 |
| 2-5-815 | O | N—Ph | methyl | H | b2 |
| 2-5-816 | O | N—Ph | b1 | H | b2 |
| 2-5-817 | O | N—Ph | b2 | H | b2 |
| 2-5-818 | O | N—Ph | b3 | H | b2 |
| 2-5-819 | O | N—Ph | H | H | b3 |
| 2-5-820 | O | N—Ph | t-bu | H | b3 |
| 2-5-821 | O | N—Ph | methyl | H | b3 |
| 2-5-822 | O | N—Ph | b1 | H | b3 |
| 2-5-823 | O | N—Ph | b2 | H | b3 |
| 2-5-824 | O | N—Ph | b3 | H | b3 |
| 2-5-825 | O | N—Ph | H | H | b4 |
| 2-5-826 | O | N—Ph | t-bu | H | b4 |
| 2-5-827 | O | N—Ph | methyl | H | b4 |
| 2-5-828 | O | N—Ph | b1 | H | b4 |
| 2-5-829 | O | N—Ph | b2 | H | b4 |
| 2-5-830 | O | N—Ph | b3 | H | b4 |
| 2-5-831 | O | N—Ph | H | H | b5 |
| 2-5-832 | O | N—Ph | t-bu | H | b5 |
| 2-5-833 | O | N—Ph | methyl | H | b5 |
| 2-5-834 | O | N—Ph | b1 | H | b5 |
| 2-5-835 | O | N—Ph | b2 | H | b5 |
| 2-5-836 | O | N—Ph | b3 | H | b5 |
| 2-5-837 | O | N—Ph | H | H | b6 |
| 2-5-838 | O | N—Ph | t-bu | H | b6 |
| 2-5-839 | O | N—Ph | methyl | H | b6 |
| 2-5-840 | O | N—Ph | b1 | H | b6 |
| 2-5-841 | O | N—Ph | b2 | H | b6 |
| 2-5-842 | O | N—Ph | b3 | H | b6 |
| 2-5-843 | O | N—Ph | H | H | b7 |
| 2-5-844 | O | N—Ph | t-bu | H | b7 |
| 2-5-845 | O | N—Ph | methyl | H | b7 |
| 2-5-846 | O | N—Ph | b1 | H | b7 |
| 2-5-847 | O | N—Ph | b2 | H | b7 |
| 2-5-848 | O | N—Ph | b3 | H | b7 |
| 2-5-849 | O | N—Ph | H | H | b8 |
| 2-5-850 | O | N—Ph | t-bu | H | b8 |
| 2-5-851 | O | N—Ph | methyl | H | b8 |
| 2-5-852 | O | N—Ph | b1 | H | b8 |
| 2-5-853 | O | N—Ph | b2 | H | b8 |
| 2-5-854 | O | N—Ph | b3 | H | b8 |
| 2-5-855 | O | N—Ph | H | H | b9 |
| 2-5-856 | O | N—Ph | t-bu | H | b9 |
| 2-5-857 | O | N—Ph | methyl | H | b9 |
| 2-5-858 | O | N—Ph | b1 | H | b9 |
| 2-5-859 | O | N—Ph | b2 | H | b9 |
| 2-5-860 | O | N—Ph | b3 | H | b9 |
| 2-5-861 | O | N—Ph | H | H | b10 |
| 2-5-862 | O | N—Ph | t-bu | H | b10 |
| 2-5-863 | O | N—Ph | methyl | H | b10 |
| 2-5-864 | O | N—Ph | b1 | H | b10 |
| 2-5-865 | O | N—Ph | b2 | H | b10 |
| 2-5-866 | O | N—Ph | b3 | H | b10 |
| 2-5-867 | O | N—Ph | H | a1 | H |
| 2-5-868 | O | N—Ph | t-bu | a1 | H |
| 2-5-869 | O | N—Ph | methyl | a1 | H |
| 2-5-870 | O | N—Ph | b1 | a1 | H |
| 2-5-871 | O | N—Ph | b2 | a1 | H |
| 2-5-872 | O | N—Ph | b3 | a1 | H |
| 2-5-873 | O | N—Ph | H | a2 | H |
| 2-5-874 | O | N—Ph | t-bu | a2 | H |
| 2-5-875 | O | N—Ph | methyl | a2 | H |
| 2-5-876 | O | N—Ph | b1 | a2 | H |
| 2-5-877 | O | N—Ph | b2 | a2 | H |
| 2-5-878 | O | N—Ph | b3 | a2 | H |
| 2-5-879 | O | N—Ph | H | a3 | H |
| 2-5-880 | O | N—Ph | t-bu | a3 | H |
| 2-5-881 | O | N—Ph | methyl | a3 | H |
| 2-5-882 | O | N—Ph | b1 | a3 | H |
| 2-5-883 | O | N—Ph | b2 | a3 | H |
| 2-5-884 | O | N—Ph | b3 | a3 | H |
| 2-5-885 | O | N—Ph | H | a4 | H |
| 2-5-886 | O | N—Ph | t-bu | a4 | H |
| 2-5-887 | O | N—Ph | methyl | a4 | H |
| 2-5-888 | O | N—Ph | b1 | a4 | H |
| 2-5-889 | O | N—Ph | b2 | a4 | H |
| 2-5-890 | O | N—Ph | b3 | a4 | H |
| 2-5-891 | O | N—Ph | H | a5 | H |
| 2-5-892 | O | N—Ph | t-bu | a5 | H |
| 2-5-893 | O | N—Ph | methyl | a5 | H |
| 2-5-894 | O | N—Ph | b1 | a5 | H |
| 2-5-895 | O | N—Ph | b2 | a5 | H |
| 2-5-896 | O | N—Ph | b3 | a5 | H |
| 2-5-897 | O | N—Ph | H | a6 | H |
| 2-5-898 | O | N—Ph | t-bu | a6 | H |
| 2-5-899 | O | N—Ph | methyl | a6 | H |
| 2-5-900 | O | N—Ph | b1 | a6 | H |
| 2-5-901 | O | N—Ph | b2 | a6 | H |
| 2-5-902 | O | N—Ph | b3 | a6 | H |
| 2-5-903 | O | N—Ph | H | b1 | H |
| 2-5-904 | O | N—Ph | t-bu | b1 | H |
| 2-5-905 | O | N—Ph | methyl | b1 | H |
| 2-5-906 | O | N—Ph | b1 | b1 | H |
| 2-5-907 | O | N—Ph | b2 | b1 | H |
| 2-5-908 | O | N—Ph | b3 | b1 | H |
| 2-5-909 | O | N—Ph | H | b2 | H |
| 2-5-910 | O | N—Ph | t-bu | b2 | H |
| 2-5-911 | O | N—Ph | methyl | b2 | H |
| 2-5-912 | O | N—Ph | b1 | b2 | H |
| 2-5-913 | O | N—Ph | b2 | b2 | H |
| 2-5-914 | O | N—Ph | b3 | b2 | H |
| 2-5-915 | O | N—Ph | H | b3 | H |
| 2-5-916 | O | N—Ph | t-bu | b3 | H |
| 2-5-917 | O | N—Ph | methyl | b3 | H |
| 2-5-918 | O | N—Ph | b1 | b3 | H |
| 2-5-919 | O | N—Ph | b2 | b3 | H |
| 2-5-920 | O | N—Ph | b3 | b3 | H |
| 2-5-921 | O | N—Ph | H | b4 | H |
| 2-5-922 | O | N—Ph | t-bu | b4 | H |
| 2-5-923 | O | N—Ph | methyl | b4 | H |
| 2-5-924 | O | N—Ph | b1 | b4 | H |
| 2-5-925 | O | N—Ph | b2 | b4 | H |
| 2-5-926 | O | N—Ph | b3 | b4 | H |
| 2-5-927 | O | N—Ph | H | b5 | H |
| 2-5-928 | O | N—Ph | t-bu | b5 | H |
| 2-5-929 | O | N—Ph | methyl | b5 | H |
| 2-5-930 | O | N—Ph | b1 | b5 | H |
| 2-5-931 | O | N—Ph | b2 | b5 | H |
| 2-5-932 | O | N—Ph | b3 | b5 | H |
| 2-5-933 | O | N—Ph | H | b6 | H |
| 2-5-934 | O | N—Ph | t-bu | b6 | H |
| 2-5-935 | O | N—Ph | methyl | b6 | H |
| 2-5-936 | O | N—Ph | b1 | b6 | H |
| 2-5-937 | O | N—Ph | b2 | b6 | H |
| 2-5-938 | O | N—Ph | b3 | b6 | H |
| 2-5-939 | O | N—Ph | H | b7 | H |
| 2-5-940 | O | N—Ph | t-bu | b7 | H |
| 2-5-941 | O | N—Ph | methyl | b7 | H |
| 2-5-942 | O | N—Ph | b1 | b7 | H |
| 2-5-943 | O | N—Ph | b2 | b7 | H |
| 2-5-944 | O | N—Ph | b3 | b7 | H |
| 2-5-945 | O | N—Ph | H | b8 | H |
| 2-5-946 | O | N—Ph | t-bu | b8 | H |
| 2-5-947 | O | N—Ph | methyl | b8 | H |
| 2-5-948 | O | N—Ph | b1 | b8 | H |
| 2-5-949 | O | N—Ph | b2 | b8 | H |
| 2-5-950 | O | N—Ph | b3 | b8 | H |
| 2-5-951 | O | N—Ph | H | b9 | H |
| 2-5-952 | O | N—Ph | t-bu | b9 | H |
| 2-5-953 | O | N—Ph | methyl | b9 | H |
| 2-5-954 | O | N—Ph | b1 | b9 | H |
| 2-5-955 | O | N—Ph | b2 | b9 | H |
| 2-5-956 | O | N—Ph | b3 | b9 | H |
| 2-5-957 | O | N—Ph | H | b10 | H |
| 2-5-958 | O | N—Ph | t-bu | b10 | H |
| 2-5-959 | O | N—Ph | methyl | b10 | H |
| 2-5-960 | O | N—Ph | b1 | b10 | H |
| 2-5-961 | O | N—Ph | b2 | b10 | H |
| 2-5-962 | O | N—Ph | b3 | b10 | H |
| 2-5-963 | O | N—Ph | H | a1 | a1 |
| 2-5-964 | O | N—Ph | t-bu | a1 | a1 |
| 2-5-965 | O | N—Ph | methyl | a1 | a1 |
| 2-5-966 | O | N—Ph | b1 | a1 | a1 |
| 2-5-967 | O | N—Ph | b2 | a1 | a1 |
| 2-5-968 | O | N—Ph | b3 | a1 | a1 |
| 2-5-969 | O | N—Ph | H | a2 | a2 |
| 2-5-970 | O | N—Ph | t-bu | a2 | a2 |
| 2-5-971 | O | N—Ph | methyl | a2 | a2 |
| 2-5-972 | O | N—Ph | b1 | a2 | a2 |
| 2-5-973 | O | N—Ph | b2 | a2 | a2 |
| 2-5-974 | O | N—Ph | b3 | a2 | a2 |
| 2-5-975 | O | N—Ph | H | a3 | a3 |
| 2-5-976 | O | N—Ph | t-bu | a3 | a3 |
| 2-5-977 | O | N—Ph | methyl | a3 | a3 |
| 2-5-978 | O | N—Ph | b1 | a3 | a3 |
| 2-5-979 | O | N—Ph | b2 | a3 | a3 |
| 2-5-980 | O | N—Ph | b3 | a3 | a3 |
| 2-5-981 | O | N—Ph | H | b1 | b1 |
| 2-5-982 | O | N—Ph | t-bu | b1 | b1 |
| 2-5-983 | O | N—Ph | methyl | b1 | b1 |
| 2-5-984 | O | N—Ph | b1 | b1 | b1 |
| 2-5-985 | O | N—Ph | b2 | b1 | b1 |
| 2-5-986 | O | N—Ph | b3 | b1 | b1 |
| 2-5-987 | O | N—Ph | H | b2 | b2 |
| 2-5-988 | O | N—Ph | t-bu | b2 | b2 |
| 2-5-989 | O | N—Ph | methyl | b2 | b2 |
| 2-5-990 | O | N—Ph | b1 | b2 | b2 |
| 2-5-991 | O | N—Ph | b2 | b2 | b2 |
| 2-5-992 | O | N—Ph | b3 | b2 | b2 |
| 2-5-993 | O | N—Ph | H | b3 | b3 |
| 2-5-994 | O | N—Ph | t-bu | b3 | b3 |
| 2-5-995 | O | N—Ph | methyl | b3 | b3 |
| 2-5-996 | O | N—Ph | b1 | b3 | b3 |
| 2-5-997 | O | N—Ph | b2 | b3 | b3 |
| 2-5-998 | O | N—Ph | b3 | b3 | b3 |
| 2-5-999 | O | N—Ph | H | b5 | b5 |
| 2-5-1000 | O | N—Ph | t-bu | b5 | b5 |
| 2-5-1001 | O | N—Ph | methyl | b5 | b5 |
| 2-5-1002 | O | N—Ph | b1 | b5 | b5 |
| 2-5-1003 | O | N—Ph | b2 | b5 | b5 |
| 2-5-1004 | O | N—Ph | b3 | b5 | b5 |
| 2-5-1005 | O | N—Ph | H | b6 | b6 |
| 2-5-1006 | O | N—Ph | t-bu | b6 | b6 |
| 2-5-1007 | O | N—Ph | methyl | b6 | b6 |
| 2-5-1008 | O | N—Ph | b1 | b6 | b6 |
| 2-5-1009 | O | N—Ph | b2 | b6 | b6 |
| 2-5-1010 | O | N—Ph | b3 | b6 | b6 |
| 2-5-1011 | O | N—Ph | H | b7 | b7 |
| 2-5-1012 | O | N—Ph | t-bu | b7 | b7 |
| 2-5-1013 | O | N—Ph | methyl | b7 | b7 |
| 2-5-1014 | O | N—Ph | b1 | b7 | b7 |
| 2-5-1015 | O | N—Ph | b2 | b7 | b7 |
| 2-5-1016 | O | N—Ph | b3 | b7 | b7 |
| 2-5-1017 | S | N—Ph | H | H | H |
| 2-5-1018 | S | N—Ph | t-bu | H | H |
| 2-5-1019 | S | N—Ph | methyl | H | H |
| 2-5-1020 | S | N—Ph | t-bu | t-bu | t-bu |
| 2-5-1021 | S | N—Ph | methyl | methyl | methyl |
| 2-5-1022 | S | N—Ph | b1 | H | H |
| 2-5-1023 | S | N—Ph | b2 | H | H |
| 2-5-1024 | S | N—Ph | b3 | H | H |
| 2-5-1025 | S | N—Ph | H | H | a1 |
| 2-5-1026 | S | N—Ph | t-bu | H | a1 |
| 2-5-1027 | S | N—Ph | methyl | H | a1 |
| 2-5-1028 | S | N—Ph | b1 | H | a1 |
| 2-5-1029 | S | N—Ph | b2 | H | a1 |
| 2-5-1030 | S | N—Ph | b3 | H | a1 |
| 2-5-1031 | S | N—Ph | H | H | a2 |
| 2-5-1032 | S | N—Ph | t-bu | H | a2 |
| 2-5-1033 | S | N—Ph | methyl | H | a2 |
| 2-5-1034 | S | N—Ph | b1 | H | a2 |
| 2-5-1035 | S | N—Ph | b2 | H | a2 |
| 2-5-1036 | S | N—Ph | b3 | H | a2 |
| 2-5-1037 | S | N—Ph | H | H | a3 |
| 2-5-1038 | S | N—Ph | t-bu | H | a3 |
| 2-5-1039 | S | N—Ph | methyl | H | a3 |
| 2-5-1040 | S | N—Ph | b1 | H | a3 |
| 2-5-1041 | S | N—Ph | b2 | H | a3 |
| 2-5-1042 | S | N—Ph | b3 | H | a3 |
| 2-5-1043 | S | N—Ph | H | H | a4 |
| 2-5-1044 | S | N—Ph | t-bu | H | a4 |
| 2-5-1045 | S | N—Ph | methyl | H | a4 |
| 2-5-1046 | S | N—Ph | b1 | H | a4 |
| 2-5-1047 | S | N—Ph | b2 | H | a4 |
| 2-5-1048 | S | N—Ph | b3 | H | a4 |
| 2-5-1049 | S | N—Ph | H | H | a5 |
| 2-5-1050 | S | N—Ph | t-bu | H | a5 |
| 2-5-1051 | S | N—Ph | methyl | H | a5 |
| 2-5-1052 | S | N—Ph | b1 | H | a5 |
| 2-5-1053 | S | N—Ph | b2 | H | a5 |
| 2-5-1054 | S | N—Ph | b3 | H | a5 |
| 2-5-1055 | S | N—Ph | H | H | a6 |
| 2-5-1056 | S | N—Ph | t-bu | H | a6 |
| 2-5-1057 | S | N—Ph | methyl | H | a6 |
| 2-5-1058 | S | N—Ph | b1 | H | a6 |
| 2-5-1059 | S | N—Ph | b2 | H | a6 |
| 2-5-1060 | S | N—Ph | b3 | H | a6 |
| 2-5-1061 | S | N—Ph | H | H | b1 |
| 2-5-1062 | S | N—Ph | t-bu | H | b1 |
| 2-5-1063 | S | N—Ph | methyl | H | b1 |
| 2-5-1064 | S | N—Ph | b1 | H | b1 |
| 2-5-1065 | S | N—Ph | b2 | H | b1 |
| 2-5-1066 | S | N—Ph | b3 | H | b1 |
| 2-5-1067 | S | N—Ph | H | H | b2 |
| 2-5-1068 | S | N—Ph | t-bu | H | b2 |
| 2-5-1069 | S | N—Ph | methyl | H | b2 |
| 2-5-1070 | S | N—Ph | b1 | H | b2 |
| 2-5-1071 | S | N—Ph | b2 | H | b2 |
| 2-5-1072 | S | N—Ph | b3 | H | b2 |
| 2-5-1073 | S | N—Ph | H | H | b3 |
| 2-5-1074 | S | N—Ph | t-bu | H | b3 |
| 2-5-1075 | S | N—Ph | methyl | H | b3 |
| 2-5-1076 | S | N—Ph | b1 | H | b3 |
| 2-5-1077 | S | N—Ph | b2 | H | b3 |
| 2-5-1078 | S | N—Ph | b3 | H | b3 |
| 2-5-1079 | S | N—Ph | H | H | b4 |
| 2-5-1080 | S | N—Ph | t-bu | H | b4 |
| 2-5-1081 | S | N—Ph | methyl | H | b4 |
| 2-5-1082 | S | N—Ph | b1 | H | b4 |
| 2-5-1083 | S | N—Ph | b2 | H | b4 |
| 2-5-1084 | S | N—Ph | b3 | H | b4 |
| 2-5-1085 | S | N—Ph | H | H | b5 |
| 2-5-1086 | S | N—Ph | t-bu | H | b5 |
| 2-5-1087 | S | N—Ph | methyl | H | b5 |
| 2-5-1088 | S | N—Ph | b1 | H | b5 |
| 2-5-1089 | S | N—Ph | b2 | H | b5 |
| 2-5-1090 | S | N—Ph | b3 | H | b5 |
| 2-5-1091 | S | N—Ph | H | H | b6 |
| 2-5-1092 | S | N—Ph | t-bu | H | b6 |
| 2-5-1093 | S | N—Ph | methyl | H | b6 |
| 2-5-1094 | S | N—Ph | b1 | H | b6 |
| 2-5-1095 | S | N—Ph | b2 | H | b6 |
| 2-5-1096 | S | N—Ph | b3 | H | b6 |
| 2-5-1097 | S | N—Ph | H | H | b7 |
| 2-5-1098 | S | N—Ph | t-bu | H | b7 |
| 2-5-1099 | S | N—Ph | methyl | H | b7 |
| 2-5-1100 | S | N—Ph | b1 | H | b7 |
| 2-5-1101 | S | N—Ph | b2 | H | b7 |
| 2-5-1102 | S | N—Ph | b3 | H | b7 |
| 2-5-1103 | S | N—Ph | H | H | b8 |
| 2-5-1104 | S | N—Ph | t-bu | H | b8 |
| 2-5-1105 | S | N—Ph | methyl | H | b8 |
| 2-5-1106 | S | N—Ph | b1 | H | b8 |
| 2-5-1107 | S | N—Ph | b2 | H | b8 |
| 2-5-1108 | S | N—Ph | b3 | H | b8 |
| 2-5-1109 | S | N—Ph | H | H | b9 |
| 2-5-1110 | S | N—Ph | t-bu | H | b9 |
| 2-5-1111 | S | N—Ph | methyl | H | b9 |
| 2-5-1112 | S | N—Ph | b1 | H | b9 |
| 2-5-1113 | S | N—Ph | b2 | H | b9 |
| 2-5-1114 | S | N—Ph | b3 | H | b9 |
| 2-5-1115 | S | N—Ph | H | H | b10 |
| 2-5-1116 | S | N—Ph | t-bu | H | b10 |
| 2-5-1117 | S | N—Ph | methyl | H | b10 |
| 2-5-1118 | S | N—Ph | b1 | H | b10 |
| 2-5-1119 | S | N—Ph | b2 | H | b10 |
| 2-5-1120 | S | N—Ph | b3 | H | b10 |
| 2-5-1121 | S | N—Ph | H | a1 | H |
| 2-5-1122 | S | N—Ph | t-bu | a1 | H |
| 2-5-1123 | S | N—Ph | methyl | a1 | H |
| 2-5-1124 | S | N—Ph | b1 | a1 | H |
| 2-5-1125 | S | N—Ph | b2 | a1 | H |
| 2-5-1126 | S | N—Ph | b3 | a1 | H |
| 2-5-1127 | S | N—Ph | H | a2 | H |
| 2-5-1128 | S | N—Ph | t-bu | a2 | H |
| 2-5-1129 | S | N—Ph | methyl | a2 | H |
| 2-5-1130 | S | N—Ph | b1 | a2 | H |
| 2-5-1131 | S | N—Ph | b2 | a2 | H |
| 2-5-1132 | S | N—Ph | b3 | a2 | H |
| 2-5-1133 | S | N—Ph | H | a3 | H |
| 2-5-1134 | S | N—Ph | t-bu | a3 | H |
| 2-5-1135 | S | N—Ph | methyl | a3 | H |
| 2-5-1136 | S | N—Ph | b1 | a3 | H |
| 2-5-1137 | S | N—Ph | b2 | a3 | H |
| 2-5-1138 | S | N—Ph | b3 | a3 | H |
| 2-5-1139 | S | N—Ph | H | a4 | H |
| 2-5-1140 | S | N—Ph | t-bu | a4 | H |
| 2-5-1141 | S | N—Ph | methyl | a4 | H |
| 2-5-1142 | S | N—Ph | b1 | a4 | H |
| 2-5-1143 | S | N—Ph | b2 | a4 | H |
| 2-5-1144 | S | N—Ph | b3 | a4 | H |
| 2-5-1145 | S | N—Ph | H | a5 | H |
| 2-5-1146 | S | N—Ph | t-bu | a5 | H |
| 2-5-1147 | S | N—Ph | methyl | a5 | H |
| 2-5-1148 | S | N—Ph | b1 | a5 | H |
| 2-5-1149 | S | N—Ph | b2 | a5 | H |
| 2-5-1150 | S | N—Ph | b3 | a5 | H |
| 2-5-1151 | S | N—Ph | H | a6 | H |
| 2-5-1152 | S | N—Ph | t-bu | a6 | H |
| 2-5-1153 | S | N—Ph | methyl | a6 | H |
| 2-5-1154 | S | N—Ph | b1 | a6 | H |
| 2-5-1155 | S | N—Ph | b2 | a6 | H |
| 2-5-1156 | S | N—Ph | b3 | a6 | H |
| 2-5-1157 | S | N—Ph | H | b1 | H |
| 2-5-1158 | S | N—Ph | t-bu | b1 | H |
| 2-5-1159 | S | N—Ph | methyl | b1 | H |
| 2-5-1160 | S | N—Ph | b1 | b1 | H |
| 2-5-1161 | S | N—Ph | b2 | b1 | H |
| 2-5-1162 | S | N—Ph | b3 | b1 | H |
| 2-5-1163 | S | N—Ph | H | b2 | H |
| 2-5-1164 | S | N—Ph | t-bu | b2 | H |
| 2-5-1165 | S | N—Ph | methyl | b2 | H |
| 2-5-1166 | S | N—Ph | b1 | b2 | H |
| 2-5-1167 | S | N—Ph | b2 | b2 | H |
| 2-5-1168 | S | N—Ph | b3 | b2 | H |
| 2-5-1169 | S | N—Ph | H | b3 | H |
| 2-5-1170 | S | N—Ph | t-bu | b3 | H |
| 2-5-1171 | S | N—Ph | methyl | b3 | H |
| 2-5-1172 | S | N—Ph | b1 | b3 | H |
| 2-5-1173 | S | N—Ph | b2 | b3 | H |
| 2-5-1174 | S | N—Ph | b3 | b3 | H |
| 2-5-1175 | S | N—Ph | H | b4 | H |
| 2-5-1176 | S | N—Ph | t-bu | b4 | H |
| 2-5-1177 | S | N—Ph | methyl | b4 | H |
| 2-5-1178 | S | N—Ph | b1 | b4 | H |
| 2-5-1179 | S | N—Ph | b2 | b4 | H |
| 2-5-1180 | S | N—Ph | b3 | b4 | H |
| 2-5-1181 | S | N—Ph | H | b5 | H |
| 2-5-1182 | S | N—Ph | t-bu | b5 | H |
| 2-5-1183 | S | N—Ph | methyl | b5 | H |
| 2-5-1184 | S | N—Ph | b1 | b5 | H |
| 2-5-1185 | S | N—Ph | b2 | b5 | H |
| 2-5-1186 | S | N—Ph | b3 | b5 | H |
| 2-5-1187 | S | N—Ph | H | b6 | H |
| 2-5-1188 | S | N—Ph | t-bu | b6 | H |
| 2-5-1189 | S | N—Ph | methyl | b6 | H |
| 2-5-1190 | S | N—Ph | b1 | b6 | H |
| 2-5-1191 | S | N—Ph | b2 | b6 | H |
| 2-5-1192 | S | N—Ph | b3 | b6 | H |
| 2-5-1193 | S | N—Ph | H | b7 | H |
| 2-5-1194 | S | N—Ph | t-bu | b7 | H |
| 2-5-1195 | S | N—Ph | methyl | b7 | H |
| 2-5-1196 | S | N—Ph | b1 | b7 | H |
| 2-5-1197 | S | N—Ph | b2 | b7 | H |
| 2-5-1198 | S | N—Ph | b3 | b7 | H |
| 2-5-1199 | S | N—Ph | H | b8 | H |
| 2-5-1200 | S | N—Ph | t-bu | b8 | H |
| 2-5-1201 | S | N—Ph | methyl | b8 | H |
| 2-5-1202 | S | N—Ph | b1 | b8 | H |
| 2-5-1203 | S | N—Ph | b2 | b8 | H |
| 2-5-1204 | S | N—Ph | b3 | b8 | H |
| 2-5-1205 | S | N—Ph | H | b9 | H |
| 2-5-1206 | S | N—Ph | t-bu | b9 | H |
| 2-5-1207 | S | N—Ph | methyl | b9 | H |
| 2-5-1208 | S | N—Ph | b1 | b9 | H |
| 2-5-1209 | S | N—Ph | b2 | b9 | H |
| 2-5-1210 | S | N—Ph | b3 | b9 | H |
| 2-5-1211 | S | N—Ph | H | b10 | H |
| 2-5-1212 | S | N—Ph | t-bu | b10 | H |
| 2-5-1213 | S | N—Ph | methyl | b10 | H |
| 2-5-1214 | S | N—Ph | b1 | b10 | H |
| 2-5-1215 | S | N—Ph | b2 | b10 | H |
| 2-5-1216 | S | N—Ph | b3 | b10 | H |
| 2-5-1217 | S | N—Ph | H | a1 | a1 |
| 2-5-1218 | S | N—Ph | t-bu | a1 | a1 |
| 2-5-1219 | S | N—Ph | methyl | a1 | a1 |
| 2-5-1220 | S | N—Ph | b1 | a1 | a1 |
| 2-5-1221 | S | N—Ph | b2 | a1 | a1 |
| 2-5-1222 | S | N—Ph | b3 | a1 | a1 |
| 2-5-1223 | S | N—Ph | H | a2 | a2 |
| 2-5-1224 | S | N—Ph | t-bu | a2 | a2 |
| 2-5-1225 | S | N—Ph | methyl | a2 | a2 |
| 2-5-1226 | S | N—Ph | b1 | a2 | a2 |
| 2-5-1227 | S | N—Ph | b2 | a2 | a2 |
| 2-5-1228 | S | N—Ph | b3 | a2 | a2 |
| 2-5-1229 | S | N—Ph | H | a3 | a3 |
| 2-5-1230 | S | N—Ph | t-bu | a3 | a3 |
| 2-5-1231 | S | N—Ph | methyl | a3 | a3 |
| 2-5-1232 | S | N—Ph | b1 | a3 | a3 |
| 2-5-1233 | S | N—Ph | b2 | a3 | a3 |
| 2-5-1234 | S | N—Ph | b3 | a3 | a3 |
| 2-5-1235 | S | N—Ph | H | b1 | b1 |
| 2-5-1236 | S | N—Ph | t-bu | b1 | b1 |
| 2-5-1237 | S | N—Ph | methyl | b1 | b1 |
| 2-5-1238 | S | N—Ph | b1 | b1 | b1 |
| 2-5-1239 | S | N—Ph | b2 | b1 | b1 |
| 2-5-1240 | S | N—Ph | b3 | b1 | b1 |
| 2-5-1241 | S | N—Ph | H | b2 | b2 |
| 2-5-1242 | S | N—Ph | t-bu | b2 | b2 |
| 2-5-1243 | S | N—Ph | methyl | b2 | b2 |
| 2-5-1244 | S | N—Ph | b1 | b2 | b2 |
| 2-5-1245 | S | N—Ph | b2 | b2 | b2 |
| 2-5-1246 | S | N—Ph | b3 | b2 | b2 |
| 2-5-1247 | S | N—Ph | H | b3 | b3 |
| 2-5-1248 | S | N—Ph | t-bu | b3 | b3 |
| 2-5-1249 | S | N—Ph | methyl | b3 | b3 |
| 2-5-1250 | S | N—Ph | b1 | b3 | b3 |
| 2-5-1251 | S | N—Ph | b2 | b3 | b3 |
| 2-5-1252 | S | N—Ph | b3 | b3 | b3 |
| 2-5-1253 | S | N—Ph | H | b5 | b5 |
| 2-5-1254 | S | N—Ph | t-bu | b5 | b5 |
| 2-5-1255 | S | N—Ph | methyl | b5 | b5 |
| 2-5-1256 | S | N—Ph | b1 | b5 | b5 |
| 2-5-1257 | S | N—Ph | b2 | b5 | b5 |
| 2-5-1258 | S | N—Ph | b3 | b5 | b5 |
| 2-5-1259 | S | N—Ph | H | b6 | b6 |
| 2-5-1260 | S | N—Ph | t-bu | b6 | b6 |
| 2-5-1261 | S | N—Ph | methyl | b6 | b6 |
| 2-5-1262 | S | N—Ph | b1 | b6 | b6 |
| 2-5-1263 | S | N—Ph | b2 | b6 | b6 |
| 2-5-1264 | S | N—Ph | b3 | b6 | b6 |
| 2-5-1265 | S | N—Ph | H | b7 | b7 |
| 2-5-1266 | S | N—Ph | t-bu | b7 | b7 |
| 2-5-1267 | S | N—Ph | methyl | b7 | b7 |
| 2-5-1268 | S | N—Ph | b1 | b7 | b7 |
| 2-5-1269 | S | N—Ph | b2 | b7 | b7 |
| 2-5-1270 | S | N—Ph | b3 | b7 | b7 |

### Group 2-6

The compounds are represented by the formula 2-6 below, wherein CY₃ is a benzene group, X₁ is B, and Y1, Y2, R1b, R1d, R2b, R2d, (R3)a3 are each shown in the table 6 below,

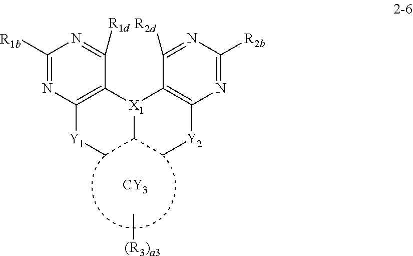

TABLE 6

| Compound | | | | | | | |
|---|---|---|---|---|---|---|---|
| No. | Y1 | Y2 | R1b | R1d | R2b | R2d | (R3)a3 |
| 2-6-001 | N—Ph | N—Ph | H | H | H | H | H |
| 2-6-002 | N—Ph | N—Ph | t-bu | H | t-bu | H | H |
| 2-6-003 | N—Ph | N—Ph | methyl | H | methyl | H | H |
| 2-6-004 | N—Ph | N—Ph | t-bu | t-bu | t-bu | t-bu | t-bu |
| 2-6-005 | N—Ph | N—Ph | methyl | methyl | methyl | methyl | methyl |
| 2-6-006 | N—Ph | N—Ph | b1 | H | b1 | H | H |
| 2-6-007 | N—Ph | N—Ph | b2 | H | b2 | H | H |
| 2-6-008 | N—Ph | N—Ph | b3 | H | b3 | H | H |
| 2-6-009 | N—Ph | N—Ph | H | H | H | H | a1 |
| 2-6-010 | N—Ph | N—Ph | t-bu | H | t-bu | H | a1 |
| 2-6-011 | N—Ph | N—Ph | methyl | H | methyl | H | a1 |
| 2-6-012 | N—Ph | N—Ph | b1 | H | b1 | H | a1 |
| 2-6-013 | N—Ph | N—Ph | b2 | H | b2 | H | a1 |
| 2-6-014 | N—Ph | N—Ph | b3 | H | b3 | H | a1 |
| 2-6-015 | N—Ph | N—Ph | H | H | H | H | a2 |
| 2-6-016 | N—Ph | N—Ph | t-bu | H | t-bu | H | a2 |
| 2-6-017 | N—Ph | N—Ph | methyl | H | methyl | H | a2 |
| 2-6-018 | N—Ph | N—Ph | b1 | H | b1 | H | a2 |
| 2-6-019 | N—Ph | N—Ph | b2 | H | b2 | H | a2 |
| 2-6-020 | N—Ph | N—Ph | b3 | H | b3 | H | a2 |
| 2-6-021 | N—Ph | N—Ph | H | H | H | H | a3 |
| 2-6-022 | N—Ph | N—Ph | t-bu | H | t-bu | H | a3 |
| 2-6-023 | N—Ph | N—Ph | methyl | H | methyl | H | a3 |
| 2-6-024 | N—Ph | N—Ph | b1 | H | b1 | H | a3 |
| 2-6-025 | N—Ph | N—Ph | b2 | H | b2 | H | a3 |
| 2-6-026 | N—Ph | N—Ph | b3 | H | b3 | H | a3 |
| 2-6-027 | N—Ph | N—Ph | H | H | H | H | a4 |
| 2-6-028 | N—Ph | N—Ph | t-bu | H | t-bu | H | a4 |
| 2-6-029 | N—Ph | N—Ph | methyl | H | methyl | H | a4 |
| 2-6-030 | N—Ph | N—Ph | b1 | H | b1 | H | a4 |
| 2-6-031 | N—Ph | N—Ph | b2 | H | b2 | H | a4 |
| 2-6-032 | N—Ph | N—Ph | b3 | H | b3 | H | a4 |
| 2-6-033 | N—Ph | N—Ph | H | H | H | H | a5 |
| 2-6-034 | N—Ph | N—Ph | t-bu | H | t-bu | H | a5 |
| 2-6-035 | N—Ph | N—Ph | methyl | H | methyl | H | a5 |
| 2-6-036 | N—Ph | N—Ph | b1 | H | b1 | H | a5 |
| 2-6-037 | N—Ph | N—Ph | b2 | H | b2 | H | a5 |
| 2-6-038 | N—Ph | N—Ph | b3 | H | b3 | H | a5 |
| 2-6-039 | N—Ph | N—Ph | H | H | H | H | a6 |
| 2-6-040 | N—Ph | N—Ph | t-bu | H | t-bu | H | a6 |
| 2-6-041 | N—Ph | N—Ph | methyl | H | methyl | H | a6 |
| 2-6-042 | N—Ph | N—Ph | b1 | H | b1 | H | a6 |
| 2-6-043 | N—Ph | N—Ph | b2 | H | b2 | H | a6 |
| 2-6-044 | N—Ph | N—Ph | b3 | H | b3 | H | a6 |
| 2-6-045 | N-tolyl | N-tolyl | H | H | H | H | H |
| 2-6-046 | N-tolyl | N-tolyl | t-bu | H | t-bu | H | H |
| 2-6-047 | N-tolyl | N-tolyl | methyl | H | methyl | H | H |
| 2-6-048 | N-tolyl | N-tolyl | t-bu | t-bu | t-bu | t-bu | t-bu |
| 2-6-049 | N-tolyl | N-tolyl | methyl | methyl | methyl | methyl | methyl |
| 2-6-050 | N-tolyl | N-tolyl | b1 | H | b1 | H | H |
| 2-6-051 | N-tolyl | N-tolyl | b2 | H | b2 | H | H |
| 2-6-052 | N-tolyl | N-tolyl | b3 | H | b3 | H | H |
| 2-6-053 | N-tolyl | N-tolyl | H | H | H | H | a1 |
| 2-6-054 | N-tolyl | N-tolyl | t-bu | H | t-bu | H | a1 |
| 2-6-055 | N-tolyl | N-tolyl | methyl | H | methyl | H | a1 |
| 2-6-056 | N-tolyl | N-tolyl | b1 | H | b1 | H | a1 |
| 2-6-057 | N-tolyl | N-tolyl | b2 | H | b2 | H | a1 |
| 2-6-058 | N-tolyl | N-tolyl | b3 | H | b3 | H | a1 |
| 2-6-059 | N-tolyl | N-tolyl | H | H | H | H | a2 |
| 2-6-060 | N-tolyl | N-tolyl | t-bu | H | t-bu | H | a2 |
| 2-6-061 | N-tolyl | N-tolyl | methyl | H | methyl | H | a2 |
| 2-6-062 | N-tolyl | N-tolyl | b1 | H | b1 | H | a2 |
| 2-6-063 | N-tolyl | N-tolyl | b2 | H | b2 | H | a2 |
| 2-6-064 | N-tolyl | N-tolyl | b3 | H | b3 | H | a2 |
| 2-6-065 | N-tolyl | N-tolyl | H | H | H | H | a3 |
| 2-6-066 | N-tolyl | N-tolyl | t-bu | H | t-bu | H | a3 |
| 2-6-067 | N-tolyl | N-tolyl | methyl | H | methyl | H | a3 |
| 2-6-068 | N-tolyl | N-tolyl | b1 | H | b1 | H | a3 |
| 2-6-069 | N-tolyl | N-tolyl | b2 | H | b2 | H | a3 |
| 2-6-070 | N-tolyl | N-tolyl | b3 | H | b3 | H | a3 |
| 2-6-071 | N-tolyl | N-tolyl | H | H | H | H | a4 |
| 2-6-072 | N-tolyl | N-tolyl | t-bu | H | t-bu | H | a4 |
| 2-6-073 | N-tolyl | N-tolyl | methyl | H | methyl | H | a4 |
| 2-6-074 | N-tolyl | N-tolyl | b1 | H | b1 | H | a4 |
| 2-6-075 | N-tolyl | N-tolyl | b2 | H | b2 | H | a4 |
| 2-6-076 | N-tolyl | N-tolyl | b3 | H | b3 | H | a4 |
| 2-6-077 | N-tolyl | N-tolyl | H | H | H | H | a5 |
| 2-6-078 | N-tolyl | N-tolyl | t-bu | H | t-bu | H | a5 |
| 2-6-079 | N-tolyl | N-tolyl | methyl | H | methyl | H | a5 |
| 2-6-080 | N-tolyl | N-tolyl | b1 | H | b1 | H | a5 |
| 2-6-081 | N-tolyl | N-tolyl | b2 | H | b2 | H | a5 |
| 2-6-082 | N-tolyl | N-tolyl | b3 | H | b3 | H | a5 |
| 2-6-083 | N-tolyl | N-tolyl | H | H | H | H | a6 |
| 2-6-084 | N-tolyl | N-tolyl | t-bu | H | t-bu | H | a6 |
| 2-6-085 | N-tolyl | N-tolyl | methyl | H | methyl | H | a6 |
| 2-6-086 | N-tolyl | N-tolyl | b1 | H | b1 | H | a6 |
| 2-6-087 | N-tolyl | N-tolyl | b2 | H | b2 | H | a6 |
| 2-6-088 | N-tolyl | N-tolyl | b3 | H | b3 | H | a6 |
| 2-6-089 | N-Biph | N-Biph | H | H | H | H | H |
| 2-6-090 | N-Biph | N-Biph | t-bu | H | t-bu | H | H |
| 2-6-091 | N-Biph | N-Biph | methyl | H | methyl | H | H |
| 2-6-092 | N-Biph | N-Biph | t-bu | t-bu | t-bu | t-bu | t-bu |
| 2-6-093 | N-Biph | N-Biph | methyl | methyl | methyl | methyl | methyl |
| 2-6-094 | N-Biph | N-Biph | b1 | H | b1 | H | H |
| 2-6-095 | N-Biph | N-Biph | b2 | H | b2 | H | H |
| 2-6-096 | N-Biph | N-Biph | b3 | H | b3 | H | H |
| 2-6-097 | N-Biph | N-Biph | H | H | H | H | a1 |
| 2-6-098 | N-Biph | N-Biph | t-bu | H | t-bu | H | a1 |
| 2-6-099 | N-Biph | N-Biph | methyl | H | methyl | H | a1 |
| 2-6-100 | N-Biph | N-Biph | b1 | H | b1 | H | a1 |
| 2-6-101 | N-Biph | N-Biph | b2 | H | b2 | H | a1 |
| 2-6-102 | N-Biph | N-Biph | b3 | H | b3 | H | a1 |
| 2-6-103 | N-Biph | N-Biph | H | H | H | H | a2 |
| 2-6-104 | N-Biph | N-Biph | t-bu | H | t-bu | H | a2 |
| 2-6-105 | N-Biph | N-Biph | methyl | H | methyl | H | a2 |
| 2-6-106 | N-Biph | N-Biph | b1 | H | b1 | H | a2 |
| 2-6-107 | N-Biph | N-Biph | b2 | H | b2 | H | a2 |
| 2-6-108 | N-Biph | N-Biph | b3 | H | b3 | H | a2 |
| 2-6-109 | N-Biph | N-Biph | H | H | H | H | a3 |
| 2-6-110 | N-Biph | N-Biph | t-bu | H | t-bu | H | a3 |
| 2-6-111 | N-Biph | N-Biph | methyl | H | methyl | H | a3 |
| 2-6-112 | N-Biph | N-Biph | b1 | H | b1 | H | a3 |
| 2-6-113 | N-Biph | N-Biph | b2 | H | b2 | H | a3 |
| 2-6-114 | N-Biph | N-Biph | b3 | H | b3 | H | a3 |
| 2-6-115 | N-Biph | N-Biph | H | H | H | H | a4 |
| 2-6-116 | N-Biph | N-Biph | t-bu | H | t-bu | H | a4 |
| 2-6-117 | N-Biph | N-Biph | methyl | H | methyl | H | a4 |
| 2-6-118 | N-Biph | N-Biph | b1 | H | b1 | H | a4 |
| 2-6-119 | N-Biph | N-Biph | b2 | H | b2 | H | a4 |
| 2-6-120 | N-Biph | N-Biph | b3 | H | b3 | H | a4 |
| 2-6-121 | N-Biph | N-Biph | H | H | H | H | a5 |
| 2-6-122 | N-Biph | N-Biph | t-bu | H | t-bu | H | a5 |
| 2-6-123 | N-Biph | N-Biph | methyl | H | methyl | H | a5 |
| 2-6-124 | N-Biph | N-Biph | b1 | H | b1 | H | a5 |
| 2-6-125 | N-Biph | N-Biph | b2 | H | b2 | H | a5 |
| 2-6-126 | N-Biph | N-Biph | b3 | H | b3 | H | a5 |
| 2-6-127 | N-Biph | N-Biph | H | H | H | H | a6 |
| 2-6-128 | N-Biph | N-Biph | t-bu | H | t-bu | H | a6 |
| 2-6-129 | N-Biph | N-Biph | methyl | H | methyl | H | a6 |
| 2-6-130 | N-Biph | N-Biph | b1 | H | b1 | H | a6 |
| 2-6-131 | N-Biph | N-Biph | b2 | H | b2 | H | a6 |
| 2-6-132 | N-Biph | N-Biph | b3 | H | b3 | H | a6 |
| 2-6-133 | O | N—Ph | H | H | H | H | H |
| 2-6-134 | O | N—Ph | t-bu | H | t-bu | H | H |
| 2-6-135 | O | N—Ph | methyl | H | methyl | H | H |
| 2-6-136 | O | N—Ph | t-bu | t-bu | t-bu | t-bu | t-bu |
| 2-6-137 | O | N—Ph | methyl | methyl | methyl | methyl | methyl |
| 2-6-138 | O | N—Ph | b1 | H | b1 | H | H |
| 2-6-139 | O | N—Ph | b2 | H | b2 | H | H |
| 2-6-140 | O | N—Ph | b3 | H | b3 | H | H |
| 2-6-141 | O | N—Ph | H | H | H | H | a1 |
| 2-6-142 | O | N—Ph | t-bu | H | t-bu | H | a1 |
| 2-6-143 | O | N—Ph | methyl | H | methyl | H | a1 |
| 2-6-144 | O | N—Ph | b1 | H | b1 | H | a1 |
| 2-6-145 | O | N—Ph | b2 | H | b2 | H | a1 |
| 2-6-146 | O | N—Ph | b3 | H | b3 | H | a1 |
| 2-6-147 | O | N—Ph | H | H | H | H | a2 |
| 2-6-148 | O | N—Ph | t-bu | H | t-bu | H | a2 |
| 2-6-149 | O | N—Ph | methyl | H | methyl | H | a2 |
| 2-6-150 | O | N—Ph | b1 | H | b1 | H | a2 |
| 2-6-151 | O | N—Ph | b2 | H | b2 | H | a2 |
| 2-6-152 | O | N—Ph | b3 | H | b3 | H | a2 |
| 2-6-153 | O | N—Ph | H | H | H | H | a3 |
| 2-6-154 | O | N—Ph | t-bu | H | t-bu | H | a3 |
| 2-6-155 | O | N—Ph | methyl | H | methyl | H | a3 |
| 2-6-156 | O | N—Ph | b1 | H | b1 | H | a3 |
| 2-6-157 | O | N—Ph | b2 | H | b2 | H | a3 |
| 2-6-158 | O | N—Ph | b3 | H | b3 | H | a3 |
| 2-6-159 | O | N—Ph | H | H | H | H | a4 |
| 2-6-160 | O | N—Ph | t-bu | H | t-bu | H | a4 |
| 2-6-161 | O | N—Ph | methyl | H | methyl | H | a4 |
| 2-6-162 | O | N—Ph | b1 | H | b1 | H | a4 |
| 2-6-163 | O | N—Ph | b2 | H | b2 | H | a4 |
| 2-6-164 | O | N—Ph | b3 | H | b3 | H | a4 |
| 2-6-165 | O | N—Ph | H | H | H | H | a5 |
| 2-6-166 | O | N—Ph | t-bu | H | t-bu | H | a5 |
| 2-6-167 | O | N—Ph | methyl | H | methyl | H | a5 |
| 2-6-168 | O | N—Ph | b1 | H | b1 | H | a5 |
| 2-6-169 | O | N—Ph | b2 | H | b2 | H | a5 |
| 2-6-170 | O | N—Ph | b3 | H | b3 | H | a5 |
| 2-6-171 | O | N—Ph | H | H | H | H | a6 |
| 2-6-172 | O | N—Ph | t-bu | H | t-bu | H | a6 |
| 2-6-173 | O | N—Ph | methyl | H | methyl | H | a6 |
| 2-6-174 | O | N—Ph | b1 | H | b1 | H | a6 |
| 2-6-175 | O | N—Ph | b2 | H | b2 | H | a6 |
| 2-6-176 | O | N—Ph | b3 | H | b3 | H | a6 |
| 2-6-177 | S | N—Ph | H | H | H | H | H |
| 2-6-178 | S | N—Ph | t-bu | H | t-bu | H | H |
| 2-6-179 | S | N—Ph | methyl | H | methyl | H | H |
| 2-6-180 | S | N—Ph | t-bu | t-bu | t-bu | t-bu | t-bu |
| 2-6-181 | S | N—Ph | methyl | methyl | methyl | methyl | methyl |
| 2-6-182 | S | N—Ph | b1 | H | b1 | H | H |
| 2-6-183 | S | N—Ph | b2 | H | b2 | H | H |
| 2-6-184 | S | N—Ph | b3 | H | b3 | H | H |
| 2-6-185 | S | N—Ph | H | H | H | H | a1 |
| 2-6-186 | S | N—Ph | t-bu | H | t-bu | H | a1 |
| 2-6-187 | S | N—Ph | methyl | H | methyl | H | a1 |
| 2-6-188 | S | N—Ph | b1 | H | b1 | H | a1 |
| 2-6-189 | S | N—Ph | b2 | H | b2 | H | a1 |
| 2-6-190 | S | N—Ph | b3 | H | b3 | H | a1 |
| 2-6-191 | S | N—Ph | H | H | H | H | a2 |
| 2-6-192 | S | N—Ph | t-bu | H | t-bu | H | a2 |
| 2-6-193 | S | N—Ph | methyl | H | methyl | H | a2 |
| 2-6-194 | S | N—Ph | b1 | H | b1 | H | a2 |
| 2-6-195 | S | N—Ph | b2 | H | b2 | H | a2 |
| 2-6-196 | S | N—Ph | b3 | H | b3 | H | a2 |
| 2-6-197 | S | N—Ph | H | H | H | H | a3 |
| 2-6-198 | S | N—Ph | t-bu | H | t-bu | H | a3 |
| 2-6-199 | S | N—Ph | methyl | H | methyl | H | a3 |
| 2-6-200 | S | N—Ph | b1 | H | b1 | H | a3 |
| 2-6-201 | S | N—Ph | b2 | H | b2 | H | a3 |
| 2-6-202 | S | N—Ph | b3 | H | b3 | H | a3 |
| 2-6-203 | S | N—Ph | H | H | H | H | a4 |
| 2-6-204 | S | N—Ph | t-bu | H | t-bu | H | a4 |
| 2-6-205 | S | N—Ph | methyl | H | methyl | H | a4 |
| 2-6-206 | S | N—Ph | b1 | H | b1 | H | a4 |
| 2-6-207 | S | N—Ph | b2 | H | b2 | H | a4 |
| 2-6-208 | S | N—Ph | b3 | H | b3 | H | a4 |
| 2-6-209 | S | N—Ph | H | H | H | H | a5 |
| 2-6-210 | S | N—Ph | t-bu | H | t-bu | H | a5 |
| 2-6-211 | S | N—Ph | methyl | H | methyl | H | a5 |
| 2-6-212 | S | N—Ph | b1 | H | b1 | H | a5 |
| 2-6-213 | S | N—Ph | b2 | H | b2 | H | a5 |
| 2-6-214 | S | N—Ph | b3 | H | b3 | H | a5 |
| 2-6-215 | S | N—Ph | H | H | H | H | a6 |
| 2-6-216 | S | N—Ph | t-bu | H | t-bu | H | a6 |
| 2-6-217 | S | N—Ph | methyl | H | methyl | H | a6 |
| 2-6-218 | S | N—Ph | b1 | H | b1 | H | a6 |
| 2-6-219 | S | N—Ph | b2 | H | b2 | H | a6 |
| 2-6-220 | S | N—Ph | b3 | H | b3 | H | a6 |

### Group 2-7

The compounds are represented by the formula 2-7 below, wherein CY₃ is a benzene group, X₁ is B, and Y1, Y2, R1b, R1d, R2a, R2c, (R3)a3 are each shown in the table 7 below,

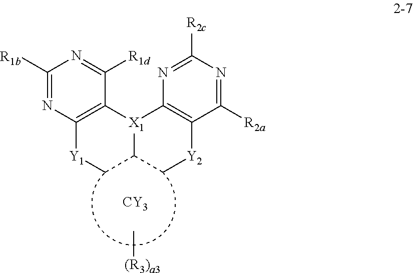

TABLE 7

| Compound | | | | | | | |
|---|---|---|---|---|---|---|---|
| No. | Y1 | Y2 | R1b | R1d | R2a | R2c | (R3)a3 |
| 2-7-001 | N—Ph | N—Ph | H | H | H | H | H |
| 2-7-002 | N—Ph | N—Ph | t-bu | H | t-bu | H | H |
| 2-7-003 | N—Ph | N—Ph | methyl | H | methyl | H | H |
| 2-7-004 | N—Ph | N—Ph | t-bu | t-bu | t-bu | t-bu | t-bu |
| 2-7-005 | N—Ph | N—Ph | methyl | methyl | methyl | methyl | methyl |
| 2-7-006 | N—Ph | N—Ph | b1 | H | b1 | H | H |
| 2-7-007 | N—Ph | N—Ph | b2 | H | b2 | H | H |
| 2-7-008 | N—Ph | N—Ph | b3 | H | b3 | H | H |
| 2-7-009 | N—Ph | N—Ph | H | H | H | H | a1 |
| 2-7-010 | N—Ph | N—Ph | t-bu | H | t-bu | t-bu | a1 |
| 2-7-011 | N—Ph | N—Ph | methyl | H | methyl | methyl | a1 |
| 2-7-012 | N—Ph | N—Ph | b1 | H | b1 | b1 | a1 |
| 2-7-013 | N—Ph | N—Ph | b2 | H | b2 | b2 | a1 |
| 2-7-014 | N—Ph | N—Ph | b3 | H | b3 | b3 | a1 |
| 2-7-015 | N—Ph | N—Ph | H | H | H | H | a2 |
| 2-7-016 | N—Ph | N—Ph | t-bu | H | t-bu | t-bu | a2 |
| 2-7-017 | N—Ph | N—Ph | methyl | H | methyl | methyl | a2 |
| 2-7-018 | N—Ph | N—Ph | b1 | H | b1 | b1 | a2 |
| 2-7-019 | N—Ph | N—Ph | b2 | H | b2 | b2 | a2 |
| 2-7-020 | N—Ph | N—Ph | b3 | H | b3 | b3 | a2 |
| 2-7-021 | N—Ph | N—Ph | H | H | H | H | a3 |
| 2-7-022 | N—Ph | N—Ph | t-bu | H | t-bu | t-bu | a3 |
| 2-7-023 | N—Ph | N—Ph | methyl | H | methyl | methyl | a3 |
| 2-7-024 | N—Ph | N—Ph | b1 | H | b1 | b1 | a3 |
| 2-7-025 | N—Ph | N—Ph | b2 | H | b2 | b2 | a3 |
| 2-7-026 | N—Ph | N—Ph | b3 | H | b3 | b3 | a3 |
| 2-7-027 | N—Ph | N—Ph | H | H | H | H | a4 |
| 2-7-028 | N—Ph | N—Ph | t-bu | H | t-bu | t-bu | a4 |
| 2-7-029 | N—Ph | N—Ph | methyl | H | methyl | methyl | a4 |
| 2-7-030 | N—Ph | N—Ph | b1 | H | b1 | b1 | a4 |
| 2-7-031 | N—Ph | N—Ph | b2 | H | b2 | b2 | a4 |
| 2-7-032 | N—Ph | N—Ph | b3 | H | b3 | b3 | a4 |
| 2-7-033 | N—Ph | N—Ph | H | H | H | H | a5 |
| 2-7-034 | N—Ph | N—Ph | t-bu | H | t-bu | t-bu | a5 |
| 2-7-035 | N—Ph | N—Ph | methyl | H | methyl | methyl | a5 |
| 2-7-036 | N—Ph | N—Ph | b1 | H | b1 | b1 | a5 |
| 2-7-037 | N—Ph | N—Ph | b2 | H | b2 | b2 | a5 |
| 2-7-038 | N—Ph | N—Ph | b3 | H | b3 | b3 | a5 |
| 2-7-039 | N—Ph | N—Ph | H | H | H | H | a6 |
| 2-7-040 | N—Ph | N—Ph | t-bu | H | t-bu | t-bu | a6 |
| 2-7-041 | N—Ph | N—Ph | methyl | H | methyl | methyl | a6 |
| 2-7-042 | N—Ph | N—Ph | b1 | H | b1 | b1 | a6 |
| 2-7-043 | N—Ph | N—Ph | b2 | H | b2 | b2 | a6 |
| 2-7-044 | N—Ph | N—Ph | b3 | H | b3 | b3 | a6 |
| 2-7-045 | N-tolyl | N-tolyl | H | H | H | H | H |
| 2-7-046 | N-tolyl | N-tolyl | t-bu | H | t-bu | H | H |
| 2-7-047 | N-tolyl | N-tolyl | methyl | H | methyl | H | H |
| 2-7-048 | N-tolyl | N-tolyl | t-bu | t-bu | t-bu | t-bu | t-bu |
| 2-7-049 | N-tolyl | N-tolyl | methyl | methyl | methyl | methyl | methyl |
| 2-7-050 | N-tolyl | N-tolyl | b1 | H | b1 | H | H |
| 2-7-051 | N-tolyl | N-tolyl | b2 | H | b2 | H | H |
| 2-7-052 | N-tolyl | N-tolyl | b3 | H | b3 | H | H |
| 2-7-053 | N-tolyl | N-tolyl | H | H | H | H | a1 |
| 2-7-054 | N-tolyl | N-tolyl | t-bu | H | t-bu | t-bu | a1 |
| 2-7-055 | N-tolyl | N-tolyl | methyl | H | methyl | methyl | a1 |
| 2-7-056 | N-tolyl | N-tolyl | b1 | H | b1 | b1 | a1 |
| 2-7-057 | N-tolyl | N-tolyl | b2 | H | b2 | b2 | a1 |
| 2-7-058 | N-tolyl | N-tolyl | b3 | H | b3 | b3 | a1 |
| 2-7-059 | N-tolyl | N-tolyl | H | H | H | H | a2 |
| 2-7-060 | N-tolyl | N-tolyl | t-bu | H | t-bu | t-bu | a2 |
| 2-7-061 | N-tolyl | N-tolyl | methyl | H | methyl | methyl | a2 |
| 2-7-062 | N-tolyl | N-tolyl | b1 | H | b1 | b1 | a2 |
| 2-7-063 | N-tolyl | N-tolyl | b2 | H | b2 | b2 | a2 |
| 2-7-064 | N-tolyl | N-tolyl | b3 | H | b3 | b3 | a2 |
| 2-7-065 | N-tolyl | N-tolyl | H | H | H | H | a3 |
| 2-7-066 | N-tolyl | N-tolyl | t-bu | H | t-bu | t-bu | a3 |
| 2-7-067 | N-tolyl | N-tolyl | methyl | H | methyl | methyl | a3 |
| 2-7-068 | N-tolyl | N-tolyl | b1 | H | b1 | b1 | a3 |
| 2-7-069 | N-tolyl | N-tolyl | b2 | H | b2 | b2 | a3 |
| 2-7-070 | N-tolyl | N-tolyl | b3 | H | b3 | b3 | a3 |
| 2-7-071 | N-tolyl | N-tolyl | H | H | H | H | a4 |
| 2-7-072 | N-tolyl | N-tolyl | t-bu | H | t-bu | t-bu | a4 |
| 2-7-073 | N-tolyl | N-tolyl | methyl | H | methyl | methyl | a4 |
| 2-7-074 | N-tolyl | N-tolyl | b1 | H | b1 | b1 | a4 |
| 2-7-075 | N-tolyl | N-tolyl | b2 | H | b2 | b2 | a4 |
| 2-7-076 | N-tolyl | N-tolyl | b3 | H | b3 | b3 | a4 |
| 2-7-077 | N-tolyl | N-tolyl | H | H | H | H | a5 |
| 2-7-078 | N-tolyl | N-tolyl | t-bu | H | t-bu | t-bu | a5 |
| 2-7-079 | N-tolyl | N-tolyl | methyl | H | methyl | methyl | a5 |
| 2-7-080 | N-tolyl | N-tolyl | b1 | H | b1 | b1 | a5 |
| 2-7-081 | N-tolyl | N-tolyl | b2 | H | b2 | b2 | a5 |
| 2-7-082 | N-tolyl | N-tolyl | b3 | H | b3 | b3 | a5 |
| 2-7-083 | N-tolyl | N-tolyl | H | H | H | H | a6 |
| 2-7-084 | N-tolyl | N-tolyl | t-bu | H | t-bu | t-bu | a6 |
| 2-7-085 | N-tolyl | N-tolyl | methyl | H | methyl | methyl | a6 |
| 2-7-086 | N-tolyl | N-tolyl | b1 | H | b1 | b1 | a6 |
| 2-7-087 | N-tolyl | N-tolyl | b2 | H | b2 | b2 | a6 |
| 2-7-088 | N-tolyl | N-tolyl | b3 | H | b3 | b3 | a6 |
| 2-7-089 | N-Biph | N-Biph | H | H | H | H | H |
| 2-7-090 | N-Biph | N-Biph | t-bu | H | t-bu | H | H |
| 2-7-091 | N-Biph | N-Biph | methyl | H | methyl | H | H |
| 2-7-092 | N-Biph | N-Biph | t-bu | t-bu | t-bu | t-bu | t-bu |
| 2-7-093 | N-Biph | N-Biph | methyl | methyl | methyl | methyl | methyl |
| 2-7-094 | N-Biph | N-Biph | b1 | H | b1 | H | H |
| 2-7-095 | N-Biph | N-Biph | b2 | H | b2 | H | H |
| 2-7-096 | N-Biph | N-Biph | b3 | H | b3 | H | H |
| 2-7-097 | N-Biph | N-Biph | H | H | H | H | a1 |
| 2-7-098 | N-Biph | N-Biph | t-bu | H | t-bu | t-bu | a1 |
| 2-7-099 | N-Biph | N-Biph | methyl | H | methyl | methyl | a1 |
| 2-7-100 | N-Biph | N-Biph | b1 | H | b1 | b1 | a1 |
| 2-7-101 | N-Biph | N-Biph | b2 | H | b2 | b2 | a1 |
| 2-7-102 | N-Biph | N-Biph | b3 | H | b3 | b3 | a1 |
| 2-7-103 | N-Biph | N-Biph | H | H | H | H | a2 |
| 2-7-104 | N-Biph | N-Biph | t-bu | H | t-bu | t-bu | a2 |
| 2-7-105 | N-Biph | N-Biph | methyl | H | methyl | methyl | a2 |
| 2-7-106 | N-Biph | N-Biph | b1 | H | b1 | b1 | a2 |
| 2-7-107 | N-Biph | N-Biph | b2 | H | b2 | b2 | a2 |
| 2-7-108 | N-Biph | N-Biph | b3 | H | b3 | b3 | a2 |
| 2-7-109 | N-Biph | N-Biph | H | H | H | H | a3 |
| 2-7-110 | N-Biph | N-Biph | t-bu | H | t-bu | t-bu | a3 |
| 2-7-111 | N-Biph | N-Biph | methyl | H | methyl | methyl | a3 |
| 2-7-112 | N-Biph | N-Biph | b1 | H | b1 | b1 | a3 |
| 2-7-113 | N-Biph | N-Biph | b2 | H | b2 | b2 | a3 |
| 2-7-114 | N-Biph | N-Biph | b3 | H | b3 | b3 | a3 |
| 2-7-115 | N-Biph | N-Biph | H | H | H | H | a4 |
| 2-7-116 | N-Biph | N-Biph | t-bu | H | t-bu | t-bu | a4 |
| 2-7-117 | N-Biph | N-Biph | methyl | H | methyl | methyl | a4 |
| 2-7-118 | N-Biph | N-Biph | b1 | H | b1 | b1 | a4 |
| 2-7-119 | N-Biph | N-Biph | b2 | H | b2 | b2 | a4 |
| 2-7-120 | N-Biph | N-Biph | b3 | H | b3 | b3 | a4 |
| 2-7-121 | N-Biph | N-Biph | H | H | H | H | a5 |
| 2-7-122 | N-Biph | N-Biph | t-bu | H | t-bu | t-bu | a5 |
| 2-7-123 | N-Biph | N-Biph | methyl | H | methyl | methyl | a5 |
| 2-7-124 | N-Biph | N-Biph | b1 | H | b1 | b1 | a5 |
| 2-7-125 | N-Biph | N-Biph | b2 | H | b2 | b2 | a5 |
| 2-7-126 | N-Biph | N-Biph | b3 | H | b3 | b3 | a5 |
| 2-7-127 | N-Biph | N-Biph | H | H | H | H | a6 |
| 2-7-128 | N-Biph | N-Biph | t-bu | H | t-bu | t-bu | a6 |
| 2-7-129 | N-Biph | N-Biph | methyl | H | methyl | methyl | a6 |
| 2-7-130 | N-Biph | N-Biph | b1 | H | b1 | b1 | a6 |
| 2-7-131 | N-Biph | N-Biph | b2 | H | b2 | b2 | a6 |
| 2-7-132 | N-Biph | N-Biph | b3 | H | b3 | b3 | a6 |
| 2-7-133 | O | N—Ph | H | H | H | H | H |
| 2-7-134 | O | N—Ph | t-bu | H | t-bu | H | H |
| 2-7-135 | O | N—Ph | methyl | H | methyl | H | H |
| 2-7-136 | O | N—Ph | t-bu | t-bu | t-bu | t-bu | t-bu |
| 2-7-137 | O | N—Ph | methyl | methyl | methyl | methyl | methyl |
| 2-7-138 | O | N—Ph | b1 | H | b1 | H | H |
| 2-7-139 | O | N—Ph | b2 | H | b2 | H | H |
| 2-7-140 | O | N—Ph | b3 | H | b3 | H | H |
| 2-7-141 | O | N—Ph | H | H | H | H | a1 |
| 2-7-142 | O | N—Ph | t-bu | H | t-bu | t-bu | a1 |
| 2-7-143 | O | N—Ph | methyl | H | methyl | methyl | a1 |
| 2-7-144 | O | N—Ph | b1 | H | b1 | b1 | a1 |
| 2-7-145 | O | N—Ph | b2 | H | b2 | b2 | a1 |
| 2-7-146 | O | N—Ph | b3 | H | b3 | b3 | a1 |
| 2-7-147 | O | N—Ph | H | H | H | H | a2 |
| 2-7-148 | O | N—Ph | t-bu | H | t-bu | t-bu | a2 |
| 2-7-149 | O | N—Ph | methyl | H | methyl | methyl | a2 |
| 2-7-150 | O | N—Ph | b1 | H | b1 | b1 | a2 |
| 2-7-151 | O | N—Ph | b2 | H | b2 | b2 | a2 |
| 2-7-152 | O | N—Ph | b3 | H | b3 | b3 | a2 |
| 2-7-153 | O | N—Ph | H | H | H | H | a3 |
| 2-7-154 | O | N—Ph | t-bu | H | t-bu | t-bu | a3 |
| 2-7-155 | O | N—Ph | methyl | H | methyl | methyl | a3 |
| 2-7-156 | O | N—Ph | b1 | H | b1 | b1 | a3 |
| 2-7-157 | O | N—Ph | b2 | H | b2 | b2 | a3 |
| 2-7-158 | O | N—Ph | b3 | H | b3 | b3 | a3 |
| 2-7-159 | O | N—Ph | H | H | H | H | a4 |
| 2-7-160 | O | N—Ph | t-bu | H | t-bu | t-bu | a4 |
| 2-7-161 | O | N—Ph | methyl | H | methyl | methyl | a4 |
| 2-7-162 | O | N—Ph | b1 | H | b1 | b1 | a4 |
| 2-7-163 | O | N—Ph | b2 | H | b2 | b2 | a4 |
| 2-7-164 | O | N—Ph | b3 | H | b3 | b3 | a4 |
| 2-7-165 | O | N—Ph | H | H | H | H | a5 |
| 2-7-166 | O | N—Ph | t-bu | H | t-bu | t-bu | a5 |
| 2-7-167 | O | N—Ph | methyl | H | methyl | methyl | a5 |
| 2-7-168 | O | N—Ph | b1 | H | b1 | b1 | a5 |
| 2-7-169 | O | N—Ph | b2 | H | b2 | b2 | a5 |
| 2-7-170 | O | N—Ph | b3 | H | b3 | b3 | a5 |
| 2-7-171 | O | N—Ph | H | H | H | H | a6 |
| 2-7-172 | O | N—Ph | t-bu | H | t-bu | t-bu | a6 |
| 2-7-173 | O | N—Ph | methyl | H | methyl | methyl | a6 |
| 2-7-174 | O | N—Ph | b1 | H | b1 | b1 | a6 |
| 2-7-175 | O | N—Ph | b2 | H | b2 | b2 | a6 |
| 2-7-176 | O | N—Ph | b3 | H | b3 | b3 | a6 |
| 2-7-177 | S | N—Ph | H | H | H | H | H |
| 2-7-178 | S | N—Ph | t-bu | H | t-bu | H | H |
| 2-7-179 | S | N—Ph | methyl | H | methyl | H | H |
| 2-7-180 | S | N—Ph | t-bu | t-bu | t-bu | t-bu | t-bu |
| 2-7-181 | S | N—Ph | methyl | methyl | methyl | methyl | methyl |
| 2-7-182 | S | N—Ph | b1 | H | b1 | H | H |
| 2-7-183 | S | N—Ph | b2 | H | b2 | H | H |
| 2-7-184 | S | N—Ph | b3 | H | b3 | H | H |
| 2-7-185 | S | N—Ph | H | H | H | H | a1 |
| 2-7-186 | S | N—Ph | t-bu | H | t-bu | t-bu | a1 |
| 2-7-187 | S | N—Ph | methyl | H | methyl | methyl | a1 |
| 2-7-188 | S | N—Ph | b1 | H | b1 | b1 | a1 |
| 2-7-189 | S | N—Ph | b2 | H | b2 | b2 | a1 |
| 2-7-190 | S | N—Ph | b3 | H | b3 | b3 | a1 |
| 2-7-191 | S | N—Ph | H | H | H | H | a2 |
| 2-7-192 | S | N—Ph | t-bu | H | t-bu | t-bu | a2 |
| 2-7-193 | S | N—Ph | methyl | H | methyl | methyl | a2 |
| 2-7-194 | S | N—Ph | b1 | H | b1 | b1 | a2 |
| 2-7-195 | S | N—Ph | b2 | H | b2 | b2 | a2 |
| 2-7-196 | S | N—Ph | b3 | H | b3 | b3 | a2 |
| 2-7-197 | S | N—Ph | H | H | H | H | a3 |
| 2-7-198 | S | N—Ph | t-bu | H | t-bu | t-bu | a3 |
| 2-7-199 | S | N—Ph | methyl | H | methyl | methyl | a3 |
| 2-7-200 | S | N—Ph | b1 | H | b1 | b1 | a3 |
| 2-7-201 | S | N—Ph | b2 | H | b2 | b2 | a3 |
| 2-7-202 | S | N—Ph | b3 | H | b3 | b3 | a3 |
| 2-7-203 | S | N—Ph | H | H | H | H | a4 |
| 2-7-204 | S | N—Ph | t-bu | H | t-bu | t-bu | a4 |
| 2-7-205 | S | N—Ph | methyl | H | methyl | methyl | a4 |
| 2-7-206 | S | N—Ph | b1 | H | b1 | b1 | a4 |
| 2-7-207 | S | N—Ph | b2 | H | b2 | b2 | a4 |
| 2-7-208 | S | N—Ph | b3 | H | b3 | b3 | a4 |
| 2-7-209 | S | N—Ph | H | H | H | H | a5 |
| 2-7-210 | S | N—Ph | t-bu | H | t-bu | t-bu | a5 |
| 2-7-211 | S | N—Ph | methyl | H | methyl | methyl | a5 |
| 2-7-212 | S | N—Ph | b1 | H | b1 | b1 | a5 |
| 2-7-213 | S | N—Ph | b2 | H | b2 | b2 | a5 |
| 2-7-214 | S | N—Ph | b3 | H | b3 | b3 | a5 |
| 2-7-215 | S | N—Ph | H | H | H | H | a6 |
| 2-7-216 | S | N—Ph | t-bu | H | t-bu | t-bu | a6 |
| 2-7-217 | S | N—Ph | methyl | H | methyl | methyl | a6 |
| 2-7-218 | S | N—Ph | b1 | H | b1 | b1 | a6 |
| 2-7-219 | S | N—Ph | b2 | H | b2 | b2 | a6 |
| 2-7-220 | S | N—Ph | b3 | H | b3 | b3 | a6 |

### Group 2-8

The compounds are represented by the formula 2-8 below, wherein CY₃ is a benzene group, X₁ is B, and Y1, Y2, R1a, R1c, R2b, R2d, (R3)a3 are each shown in the table 8 below,

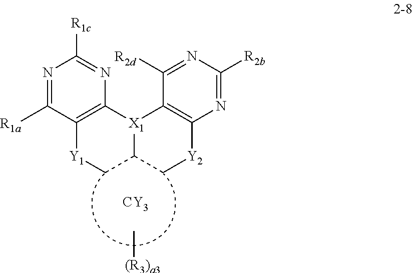

TABLE 8

| Compound | | | | | | | |
|---|---|---|---|---|---|---|---|
| No. | Y1 | Y2 | R1a | R1c | R2b | R2d | (R3)a3 |
| 2-8-001 | N—Ph | N—Ph | H | H | H | H | H |
| 2-8-002 | N—Ph | N—Ph | t-bu | H | t-bu | H | H |
| 2-8-003 | N—Ph | N—Ph | methyl | H | methyl | H | H |
| 2-8-004 | N—Ph | N—Ph | t-bu | t-bu | t-bu | t-bu | t-bu |
| 2-8-005 | N—Ph | N—Ph | methyl | methyl | methyl | methyl | methyl |
| 2-8-006 | N—Ph | N—Ph | b1 | H | b1 | H | H |
| 2-8-007 | N—Ph | N—Ph | b2 | H | b2 | H | H |
| 2-8-008 | N—Ph | N—Ph | b3 | H | b3 | H | H |
| 2-8-009 | N—Ph | N—Ph | H | H | H | H | a1 |
| 2-8-010 | N—Ph | N—Ph | t-bu | t-bu | t-bu | H | a1 |
| 2-8-011 | N—Ph | N—Ph | methyl | methyl | methyl | H | a1 |
| 2-8-012 | N—Ph | N—Ph | b1 | b1 | b1 | H | a1 |
| 2-8-013 | N—Ph | N—Ph | b2 | b2 | b2 | H | a1 |
| 2-8-014 | N—Ph | N—Ph | b3 | b3 | b3 | H | a1 |
| 2-8-015 | N—Ph | N—Ph | H | H | H | H | a2 |
| 2-8-016 | N—Ph | N—Ph | t-bu | t-bu | t-bu | H | a2 |
| 2-8-017 | N—Ph | N—Ph | methyl | methyl | methyl | H | a2 |
| 2-8-018 | N—Ph | N—Ph | b1 | b1 | b1 | H | a2 |
| 2-8-019 | N—Ph | N—Ph | b2 | b2 | b2 | H | a2 |
| 2-8-020 | N—Ph | N—Ph | b3 | b3 | b3 | H | a2 |
| 2-8-021 | N—Ph | N—Ph | H | H | H | H | a3 |
| 2-8-022 | N—Ph | N—Ph | t-bu | t-bu | t-bu | H | a3 |
| 2-8-023 | N—Ph | N—Ph | methyl | methyl | methyl | H | a3 |
| 2-8-024 | N—Ph | N—Ph | b1 | b1 | b1 | H | a3 |
| 2-8-025 | N—Ph | N—Ph | b2 | b2 | b2 | H | a3 |
| 2-8-026 | N—Ph | N—Ph | b3 | b3 | b3 | H | a3 |
| 2-8-027 | N—Ph | N—Ph | H | H | H | H | a4 |
| 2-8-028 | N—Ph | N—Ph | t-bu | t-bu | t-bu | H | a4 |
| 2-8-029 | N—Ph | N—Ph | methyl | methyl | methyl | H | a4 |
| 2-8-030 | N—Ph | N—Ph | b1 | b1 | b1 | H | a4 |
| 2-8-031 | N—Ph | N—Ph | b2 | b2 | b2 | H | a4 |
| 2-8-032 | N—Ph | N—Ph | b3 | b3 | b3 | H | a4 |
| 2-8-033 | N—Ph | N—Ph | H | H | H | H | a5 |
| 2-8-034 | N—Ph | N—Ph | t-bu | t-bu | t-bu | H | a5 |
| 2-8-035 | N—Ph | N—Ph | methyl | methyl | methyl | H | a5 |
| 2-8-036 | N—Ph | N—Ph | b1 | b1 | b1 | H | a5 |
| 2-8-037 | N—Ph | N—Ph | b2 | b2 | b2 | H | a5 |
| 2-8-038 | N—Ph | N—Ph | b3 | b3 | b3 | H | a5 |
| 2-8-039 | N—Ph | N—Ph | H | H | H | H | a6 |
| 2-8-040 | N—Ph | N—Ph | t-bu | t-bu | t-bu | H | a6 |
| 2-8-041 | N—Ph | N—Ph | methyl | methyl | methyl | H | a6 |
| 2-8-042 | N—Ph | N—Ph | b1 | b1 | b1 | H | a6 |
| 2-8-043 | N—Ph | N—Ph | b2 | b2 | b2 | H | a6 |
| 2-8-044 | N—Ph | N—Ph | b3 | b3 | b3 | H | a6 |
| 2-8-045 | N-tolyl | N-tolyl | H | H | H | H | H |
| 2-8-046 | N-tolyl | N-tolyl | t-bu | H | t-bu | H | H |
| 2-8-047 | N-tolyl | N-tolyl | methyl | H | methyl | H | H |
| 2-8-048 | N-tolyl | N-tolyl | t-bu | t-bu | t-bu | t-bu | t-bu |
| 2-8-049 | N-tolyl | N-tolyl | methyl | methyl | methyl | methyl | methyl |
| 2-8-050 | N-tolyl | N-tolyl | b1 | H | b1 | H | H |
| 2-8-051 | N-tolyl | N-tolyl | b2 | H | b2 | H | H |
| 2-8-052 | N-tolyl | N-tolyl | b3 | H | b3 | H | H |
| 2-8-053 | N-tolyl | N-tolyl | H | H | H | H | a1 |
| 2-8-054 | N-tolyl | N-tolyl | t-bu | t-bu | t-bu | H | a1 |
| 2-8-055 | N-tolyl | N-tolyl | methyl | methyl | methyl | H | a1 |
| 2-8-056 | N-tolyl | N-tolyl | b1 | b1 | b1 | H | a1 |
| 2-8-057 | N-tolyl | N-tolyl | b2 | b2 | b2 | H | a1 |
| 2-8-058 | N-tolyl | N-tolyl | b3 | b3 | b3 | H | a1 |
| 2-8-059 | N-tolyl | N-tolyl | H | H | H | H | a2 |
| 2-8-060 | N-tolyl | N-tolyl | t-bu | t-bu | t-bu | H | a2 |
| 2-8-061 | N-tolyl | N-tolyl | methyl | methyl | methyl | H | a2 |
| 2-8-062 | N-tolyl | N-tolyl | b1 | b1 | b1 | H | a2 |
| 2-8-063 | N-tolyl | N-tolyl | b2 | b2 | b2 | H | a2 |
| 2-8-064 | N-tolyl | N-tolyl | b3 | b3 | b3 | H | a2 |
| 2-8-065 | N-tolyl | N-tolyl | H | H | H | H | a3 |
| 2-8-066 | N-tolyl | N-tolyl | t-bu | t-bu | t-bu | H | a3 |
| 2-8-067 | N-tolyl | N-tolyl | methyl | methyl | methyl | H | a3 |
| 2-8-068 | N-tolyl | N-tolyl | b1 | b1 | b1 | H | a3 |
| 2-8-069 | N-tolyl | N-tolyl | b2 | b2 | b2 | H | a3 |
| 2-8-070 | N-tolyl | N-tolyl | b3 | b3 | b3 | H | a3 |
| 2-8-071 | N-tolyl | N-tolyl | H | H | H | H | a4 |
| 2-8-072 | N-tolyl | N-tolyl | t-bu | t-bu | t-bu | H | a4 |
| 2-8-073 | N-tolyl | N-tolyl | methyl | methyl | methyl | H | a4 |
| 2-8-074 | N-tolyl | N-tolyl | b1 | b1 | b1 | H | a4 |
| 2-8-075 | N-tolyl | N-tolyl | b2 | b2 | b2 | H | a4 |
| 2-8-076 | N-tolyl | N-tolyl | b3 | b3 | b3 | H | a4 |
| 2-8-077 | N-tolyl | N-tolyl | H | H | H | H | a5 |
| 2-8-078 | N-tolyl | N-tolyl | t-bu | t-bu | t-bu | H | a5 |
| 2-8-079 | N-tolyl | N-tolyl | methyl | methyl | methyl | H | a5 |
| 2-8-080 | N-tolyl | N-tolyl | b1 | b1 | b1 | H | a5 |
| 2-8-081 | N-tolyl | N-tolyl | b2 | b2 | b2 | H | a5 |
| 2-8-082 | N-tolyl | N-tolyl | b3 | b3 | b3 | H | a5 |
| 2-8-083 | N-tolyl | N-tolyl | H | H | H | H | a6 |
| 2-8-084 | N-tolyl | N-tolyl | t-bu | t-bu | t-bu | H | a6 |
| 2-8-085 | N-tolyl | N-tolyl | methyl | methyl | methyl | H | a6 |
| 2-8-086 | N-tolyl | N-tolyl | b1 | b1 | b1 | H | a6 |
| 2-8-087 | N-tolyl | N-tolyl | b2 | b2 | b2 | H | a6 |
| 2-8-088 | N-tolyl | N-tolyl | b3 | b3 | b3 | H | a6 |
| 2-8-089 | N-Biph | N-Biph | H | H | H | H | H |
| 2-8-090 | N-Biph | N-Biph | t-bu | H | t-bu | H | H |
| 2-8-091 | N-Biph | N-Biph | methyl | H | methyl | H | H |
| 2-8-092 | N-Biph | N-Biph | t-bu | t-bu | t-bu | t-bu | t-bu |
| 2-8-093 | N-Biph | N-Biph | methyl | methyl | methyl | methyl | methyl |
| 2-8-094 | N-Biph | N-Biph | b1 | H | b1 | H | H |
| 2-8-095 | N-Biph | N-Biph | b2 | H | b2 | H | H |
| 2-8-096 | N-Biph | N-Biph | b3 | H | b3 | H | H |
| 2-8-097 | N-Biph | N-Biph | H | H | H | H | a1 |
| 2-8-098 | N-Biph | N-Biph | t-bu | t-bu | t-bu | H | a1 |
| 2-8-099 | N-Biph | N-Biph | methyl | methyl | methyl | H | a1 |
| 2-8-100 | N-Biph | N-Biph | b1 | b1 | b1 | H | a1 |
| 2-8-101 | N-Biph | N-Biph | b2 | b2 | b2 | H | a1 |
| 2-8-102 | N-Biph | N-Biph | b3 | b3 | b3 | H | a1 |
| 2-8-103 | N-Biph | N-Biph | H | H | H | H | a2 |
| 2-8-104 | N-Biph | N-Biph | t-bu | t-bu | t-bu | H | a2 |
| 2-8-105 | N-Biph | N-Biph | methyl | methyl | methyl | H | a2 |
| 2-8-106 | N-Biph | N-Biph | b1 | b1 | b1 | H | a2 |
| 2-8-107 | N-Biph | N-Biph | b2 | b2 | b2 | H | a2 |
| 2-8-108 | N-Biph | N-Biph | b3 | b3 | b3 | H | a2 |
| 2-8-109 | N-Biph | N-Biph | H | H | H | H | a3 |
| 2-8-110 | N-Biph | N-Biph | t-bu | t-bu | t-bu | H | a3 |
| 2-8-111 | N-Biph | N-Biph | methyl | methyl | methyl | H | a3 |
| 2-8-112 | N-Biph | N-Biph | b1 | b1 | b1 | H | a3 |
| 2-8-113 | N-Biph | N-Biph | b2 | b2 | b2 | H | a3 |
| 2-8-114 | N-Biph | N-Biph | b3 | b3 | b3 | H | a3 |
| 2-8-115 | N-Biph | N-Biph | H | H | H | H | a4 |
| 2-8-116 | N-Biph | N-Biph | t-bu | t-bu | t-bu | H | a4 |
| 2-8-117 | N-Biph | N-Biph | methyl | methyl | methyl | H | a4 |
| 2-8-118 | N-Biph | N-Biph | b1 | b1 | b1 | H | a4 |
| 2-8-119 | N-Biph | N-Biph | b2 | b2 | b2 | H | a4 |
| 2-8-120 | N-Biph | N-Biph | b3 | b3 | b3 | H | a4 |
| 2-8-121 | N-Biph | N-Biph | H | H | H | H | a5 |
| 2-8-122 | N-Biph | N-Biph | t-bu | t-bu | t-bu | H | a5 |
| 2-8-123 | N-Biph | N-Biph | methyl | methyl | methyl | H | a5 |
| 2-8-124 | N-Biph | N-Biph | b1 | b1 | b1 | H | a5 |
| 2-8-125 | N-Biph | N-Biph | b2 | b2 | b2 | H | a5 |
| 2-8-126 | N-Biph | N-Biph | b3 | b3 | b3 | H | a5 |
| 2-8-127 | N-Biph | N-Biph | H | H | H | H | a6 |
| 2-8-128 | N-Biph | N-Biph | t-bu | t-bu | t-bu | H | a6 |
| 2-8-129 | N-Biph | N-Biph | methyl | methyl | methyl | H | a6 |
| 2-8-130 | N-Biph | N-Biph | b1 | b1 | b1 | H | a6 |
| 2-8-131 | N-Biph | N-Biph | b2 | b2 | b2 | H | a6 |
| 2-8-132 | N-Biph | N-Biph | b3 | b3 | b3 | H | a6 |
| 2-8-133 | O | N—Ph | H | H | H | H | H |
| 2-8-134 | O | N—Ph | t-bu | H | t-bu | H | H |
| 2-8-135 | O | N—Ph | methyl | H | methyl | H | H |
| 2-8-136 | O | N—Ph | t-bu | t-bu | t-bu | t-bu | t-bu |
| 2-8-137 | O | N—Ph | methyl | methyl | methyl | methyl | methyl |
| 2-8-138 | O | N—Ph | b1 | H | b1 | H | H |
| 2-8-139 | O | N—Ph | b2 | H | b2 | H | H |
| 2-8-140 | O | N—Ph | b3 | H | b3 | H | H |
| 2-8-141 | O | N—Ph | H | H | H | H | a1 |
| 2-8-142 | O | N—Ph | t-bu | t-bu | t-bu | H | a1 |
| 2-8-143 | O | N—Ph | methyl | methyl | methyl | H | a1 |
| 2-8-144 | O | N—Ph | b1 | b1 | b1 | H | a1 |
| 2-8-145 | O | N—Ph | b2 | b2 | b2 | H | a1 |
| 2-8-146 | O | N—Ph | b3 | b3 | b3 | H | a1 |
| 2-8-147 | O | N—Ph | H | H | H | H | a2 |
| 2-8-148 | O | N—Ph | t-bu | t-bu | t-bu | H | a2 |
| 2-8-149 | O | N—Ph | methyl | methyl | methyl | H | a2 |
| 2-8-150 | O | N—Ph | b1 | b1 | b1 | H | a2 |
| 2-8-151 | O | N—Ph | b2 | b2 | b2 | H | a2 |
| 2-8-152 | O | N—Ph | b3 | b3 | b3 | H | a2 |
| 2-8-153 | O | N—Ph | H | H | H | H | a3 |
| 2-8-154 | O | N—Ph | t-bu | t-bu | t-bu | H | a3 |
| 2-8-155 | O | N—Ph | methyl | methyl | methyl | H | a3 |
| 2-8-156 | O | N—Ph | b1 | b1 | b1 | H | a3 |
| 2-8-157 | O | N—Ph | b2 | b2 | b2 | H | a3 |
| 2-8-158 | O | N—Ph | b3 | b3 | b3 | H | a3 |
| 2-8-159 | O | N—Ph | H | H | H | H | a4 |
| 2-8-160 | O | N—Ph | t-bu | t-bu | t-bu | H | a4 |
| 2-8-161 | O | N—Ph | methyl | methyl | methyl | H | a4 |
| 2-8-162 | O | N—Ph | b1 | b1 | b1 | H | a4 |
| 2-8-163 | O | N—Ph | b2 | b2 | b2 | H | a4 |
| 2-8-164 | O | N—Ph | b3 | b3 | b3 | H | a4 |
| 2-8-165 | O | N—Ph | H | H | H | H | a5 |
| 2-8-166 | O | N—Ph | t-bu | t-bu | t-bu | H | a5 |
| 2-8-167 | O | N—Ph | methyl | methyl | methyl | H | a5 |
| 2-8-168 | O | N—Ph | b1 | b1 | b1 | H | a5 |
| 2-8-169 | O | N—Ph | b2 | b2 | b2 | H | a5 |
| 2-8-170 | O | N—Ph | b3 | b3 | b3 | H | a5 |
| 2-8-171 | O | N—Ph | H | H | H | H | a6 |
| 2-8-172 | O | N—Ph | t-bu | t-bu | t-bu | H | a6 |
| 2-8-173 | O | N—Ph | methyl | methyl | methyl | H | a6 |
| 2-8-174 | O | N—Ph | b1 | b1 | b1 | H | a6 |
| 2-8-175 | O | N—Ph | b2 | b2 | b2 | H | a6 |
| 2-8-176 | O | N—Ph | b3 | b3 | b3 | H | a6 |
| 2-8-177 | S | N—Ph | H | H | H | H | H |
| 2-8-178 | S | N—Ph | t-bu | H | t-bu | H | H |
| 2-8-179 | S | N—Ph | methyl | H | methyl | H | H |
| 2-8-180 | S | N—Ph | t-bu | t-bu | t-bu | t-bu | t-bu |
| 2-8-181 | S | N—Ph | methyl | methyl | methyl | methyl | methyl |
| 2-8-182 | S | N—Ph | b1 | H | b1 | H | H |
| 2-8-183 | S | N—Ph | b2 | H | b2 | H | H |
| 2-8-184 | S | N—Ph | b3 | H | b3 | H | H |
| 2-8-185 | S | N—Ph | H | H | H | H | a1 |
| 2-8-186 | S | N—Ph | t-bu | t-bu | t-bu | H | a1 |
| 2-8-187 | S | N—Ph | methyl | methyl | methyl | H | a1 |
| 2-8-188 | S | N—Ph | b1 | b1 | b1 | H | a1 |
| 2-8-189 | S | N—Ph | b2 | b2 | b2 | H | a1 |
| 2-8-190 | S | N—Ph | b3 | b3 | b3 | H | a1 |
| 2-8-191 | S | N—Ph | H | H | H | H | a2 |
| 2-8-192 | S | N—Ph | t-bu | t-bu | t-bu | H | a2 |
| 2-8-193 | S | N—Ph | methyl | methyl | methyl | H | a2 |
| 2-8-194 | S | N—Ph | b1 | b1 | b1 | H | a2 |
| 2-8-195 | S | N—Ph | b2 | b2 | b2 | H | a2 |
| 2-8-196 | S | N—Ph | b3 | b3 | b3 | H | a2 |
| 2-8-197 | S | N—Ph | H | H | H | H | a3 |
| 2-8-198 | S | N—Ph | t-bu | t-bu | t-bu | H | a3 |
| 2-8-199 | S | N—Ph | methyl | methyl | methyl | H | a3 |
| 2-8-200 | S | N—Ph | b1 | b1 | b1 | H | a3 |
| 2-8-201 | S | N—Ph | b2 | b2 | b2 | H | a3 |
| 2-8-202 | S | N—Ph | b3 | b3 | b3 | H | a3 |
| 2-8-203 | S | N—Ph | H | H | H | H | a4 |
| 2-8-204 | S | N—Ph | t-bu | t-bu | t-bu | H | a4 |
| 2-8-205 | S | N—Ph | methyl | methyl | methyl | H | a4 |
| 2-8-206 | S | N—Ph | b1 | b1 | b1 | H | a4 |
| 2-8-207 | S | N—Ph | b2 | b2 | b2 | H | a4 |
| 2-8-208 | S | N—Ph | b3 | b3 | b3 | H | a4 |
| 2-8-209 | S | N—Ph | H | H | H | H | a5 |
| 2-8-210 | S | N—Ph | t-bu | t-bu | t-bu | H | a5 |
| 2-8-211 | S | N—Ph | methyl | methyl | methyl | H | a5 |
| 2-8-212 | S | N—Ph | b1 | b1 | b1 | H | a5 |
| 2-8-213 | S | N—Ph | b2 | b2 | b2 | H | a5 |
| 2-8-214 | S | N—Ph | b3 | b3 | b3 | H | a5 |
| 2-8-215 | S | N—Ph | H | H | H | H | a6 |
| 2-8-216 | S | N—Ph | t-bu | t-bu | t-bu | H | a6 |
| 2-8-217 | S | N—Ph | methyl | methyl | methyl | H | a6 |
| 2-8-218 | S | N—Ph | b1 | b1 | b1 | H | a6 |
| 2-8-219 | S | N—Ph | b2 | b2 | b2 | H | a6 |
| 2-8-220 | S | N—Ph | b3 | b3 | b3 | H | a6 |

### Group 2-9

The compounds are represented by the formula 2-9 below, wherein CY₃ is a benzene group, X₁ is B, and Y1, Y2, R1a, R1c, R2a, R2c, (R3)a3 are each shown in the table 9 below,

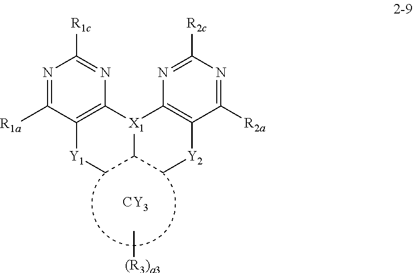

TABLE 9

| Compound | | | | | | | |
|---|---|---|---|---|---|---|---|
| No. | Y1 | Y2 | R1a | R1c | R2a | R2c | (R3)a3 |
| 2-9-001 | N—Ph | N—Ph | H | H | H | H | H |
| 2-9-002 | N—Ph | N—Ph | t-bu | H | t-bu | H | H |
| 2-9-003 | N—Ph | N—Ph | methyl | H | methyl | H | H |
| 2-9-004 | N—Ph | N—Ph | t-bu | t-bu | t-bu | t-bu | t-bu |
| 2-9-005 | N—Ph | N—Ph | methyl | methyl | methyl | methyl | methyl |
| 2-9-006 | N—Ph | N—Ph | b1 | H | b1 | H | H |
| 2-9-007 | N—Ph | N—Ph | b2 | H | b2 | H | H |
| 2-9-008 | N—Ph | N—Ph | b3 | H | b3 | H | H |
| 2-9-009 | N—Ph | N—Ph | H | H | H | H | a1 |
| 2-9-010 | N—Ph | N—Ph | t-bu | t-bu | t-bu | t-bu | a1 |
| 2-9-011 | N—Ph | N—Ph | methyl | methyl | methyl | methyl | a1 |
| 2-9-012 | N—Ph | N—Ph | b1 | b1 | b1 | b1 | a1 |
| 2-9-013 | N—Ph | N—Ph | b2 | b2 | b2 | b2 | a1 |
| 2-9-014 | N—Ph | N—Ph | b3 | b3 | b3 | b3 | a1 |
| 2-9-015 | N—Ph | N—Ph | H | H | H | H | a2 |
| 2-9-016 | N—Ph | N—Ph | t-bu | t-bu | t-bu | t-bu | a2 |
| 2-9-017 | N—Ph | N—Ph | methyl | methyl | methyl | methyl | a2 |
| 2-9-018 | N—Ph | N—Ph | b1 | b1 | b1 | b1 | a2 |
| 2-9-019 | N—Ph | N—Ph | b2 | b2 | b2 | b2 | a2 |
| 2-9-020 | N—Ph | N—Ph | b3 | b3 | b3 | b3 | a2 |
| 2-9-021 | N—Ph | N—Ph | H | H | H | H | a3 |
| 2-9-022 | N—Ph | N—Ph | t-bu | t-bu | t-bu | t-bu | a3 |
| 2-9-023 | N—Ph | N—Ph | methyl | methyl | methyl | methyl | a3 |
| 2-9-024 | N—Ph | N—Ph | b1 | b1 | b1 | b1 | a3 |
| 2-9-025 | N—Ph | N—Ph | b2 | b2 | b2 | b2 | a3 |
| 2-9-026 | N—Ph | N—Ph | b3 | b3 | b3 | b3 | a3 |
| 2-9-027 | N—Ph | N—Ph | H | H | H | H | a4 |
| 2-9-028 | N—Ph | N—Ph | t-bu | t-bu | t-bu | t-bu | a4 |
| 2-9-029 | N—Ph | N—Ph | methyl | methyl | methyl | methyl | a4 |
| 2-9-030 | N—Ph | N—Ph | b1 | b1 | b1 | b1 | a4 |
| 2-9-031 | N—Ph | N—Ph | b2 | b2 | b2 | b2 | a4 |
| 2-9-032 | N—Ph | N—Ph | b3 | b3 | b3 | b3 | a4 |
| 2-9-033 | N—Ph | N—Ph | H | H | H | H | a5 |
| 2-9-034 | N—Ph | N—Ph | t-bu | t-bu | t-bu | t-bu | a5 |
| 2-9-035 | N—Ph | N—Ph | methyl | methyl | methyl | methyl | a5 |
| 2-9-036 | N—Ph | N—Ph | b1 | b1 | b1 | b1 | a5 |
| 2-9-037 | N—Ph | N—Ph | b2 | b2 | b2 | b2 | a5 |
| 2-9-038 | N—Ph | N—Ph | b3 | b3 | b3 | b3 | a5 |
| 2-9-039 | N—Ph | N—Ph | H | H | H | H | a6 |
| 2-9-040 | N—Ph | N—Ph | t-bu | t-bu | t-bu | t-bu | a6 |
| 2-9-041 | N—Ph | N—Ph | methyl | methyl | methyl | methyl | a6 |
| 2-9-042 | N—Ph | N—Ph | b1 | b1 | b1 | b1 | a6 |
| 2-9-043 | N—Ph | N—Ph | b2 | b2 | b2 | b2 | a6 |
| 2-9-044 | N—Ph | N—Ph | b3 | b3 | b3 | b3 | a6 |
| 2-9-045 | N-tolyl | N-tolyl | H | H | H | H | H |
| 2-9-046 | N-tolyl | N-tolyl | t-bu | H | t-bu | H | H |
| 2-9-047 | N-tolyl | N-tolyl | methyl | H | methyl | H | H |
| 2-9-048 | N-tolyl | N-tolyl | t-bu | t-bu | t-bu | t-bu | t-bu |
| 2-9-049 | N-tolyl | N-tolyl | methyl | methyl | methyl | methyl | methyl |
| 2-9-050 | N-tolyl | N-tolyl | b1 | H | b1 | H | H |
| 2-9-051 | N-tolyl | N-tolyl | b2 | H | b2 | H | H |
| 2-9-052 | N-tolyl | N-tolyl | b3 | H | b3 | H | H |
| 2-9-053 | N-tolyl | N-tolyl | H | H | H | H | a1 |
| 2-9-054 | N-tolyl | N-tolyl | t-bu | t-bu | t-bu | t-bu | a1 |
| 2-9-055 | N-tolyl | N-tolyl | methyl | methyl | methyl | methyl | a1 |
| 2-9-056 | N-tolyl | N-tolyl | b1 | b1 | b1 | b1 | a1 |
| 2-9-057 | N-tolyl | N-tolyl | b2 | b2 | b2 | b2 | a1 |
| 2-9-058 | N-tolyl | N-tolyl | b3 | b3 | b3 | b3 | a1 |
| 2-9-059 | N-tolyl | N-tolyl | H | H | H | H | a2 |
| 2-9-060 | N-tolyl | N-tolyl | t-bu | t-bu | t-bu | t-bu | a2 |
| 2-9-061 | N-tolyl | N-tolyl | methyl | methyl | methyl | methyl | a2 |
| 2-9-062 | N-tolyl | N-tolyl | b1 | b1 | b1 | b1 | a2 |
| 2-9-063 | N-tolyl | N-tolyl | b2 | b2 | b2 | b2 | a2 |
| 2-9-064 | N-tolyl | N-tolyl | b3 | b3 | b3 | b3 | a2 |
| 2-9-065 | N-tolyl | N-tolyl | H | H | H | H | a3 |
| 2-9-066 | N-tolyl | N-tolyl | t-bu | t-bu | t-bu | t-bu | a3 |
| 2-9-067 | N-tolyl | N-tolyl | methyl | methyl | methyl | methyl | a3 |
| 2-9-068 | N-tolyl | N-tolyl | b1 | b1 | b1 | b1 | a3 |
| 2-9-069 | N-tolyl | N-tolyl | b2 | b2 | b2 | b2 | a3 |
| 2-9-070 | N-tolyl | N-tolyl | b3 | b3 | b3 | b3 | a3 |
| 2-9-071 | N-tolyl | N-tolyl | H | H | H | H | a4 |
| 2-9-072 | N-tolyl | N-tolyl | t-bu | t-bu | t-bu | t-bu | a4 |
| 2-9-073 | N-tolyl | N-tolyl | methyl | methyl | methyl | methyl | a4 |
| 2-9-074 | N-tolyl | N-tolyl | b1 | b1 | b1 | b1 | a4 |
| 2-9-075 | N-tolyl | N-tolyl | b2 | b2 | b2 | b2 | a4 |
| 2-9-076 | N-tolyl | N-tolyl | b3 | b3 | b3 | b3 | a4 |
| 2-9-077 | N-tolyl | N-tolyl | H | H | H | H | a5 |
| 2-9-078 | N-tolyl | N-tolyl | t-bu | t-bu | t-bu | t-bu | a5 |
| 2-9-079 | N-tolyl | N-tolyl | methyl | methyl | methyl | methyl | a5 |
| 2-9-080 | N-tolyl | N-tolyl | b1 | b1 | b1 | b1 | a5 |
| 2-9-081 | N-tolyl | N-tolyl | b2 | b2 | b2 | b2 | a5 |
| 2-9-082 | N-tolyl | N-tolyl | b3 | b3 | b3 | b3 | a5 |
| 2-9-083 | N-tolyl | N-tolyl | H | H | H | H | a6 |
| 2-9-084 | N-tolyl | N-tolyl | t-bu | t-bu | t-bu | t-bu | a6 |
| 2-9-085 | N-tolyl | N-tolyl | methyl | methyl | methyl | methyl | a6 |
| 2-9-086 | N-tolyl | N-tolyl | b1 | b1 | b1 | b1 | a6 |
| 2-9-087 | N-tolyl | N-tolyl | b2 | b2 | b2 | b2 | a6 |
| 2-9-088 | N-tolyl | N-tolyl | b3 | b3 | b3 | b3 | a6 |
| 2-9-089 | N-Biph | N-Biph | H | H | H | H | H |
| 2-9-090 | N-Biph | N-Biph | t-bu | H | t-bu | H | H |
| 2-9-091 | N-Biph | N-Biph | methyl | H | methyl | H | H |
| 2-9-092 | N-Biph | N-Biph | t-bu | t-bu | t-bu | t-bu | t-bu |
| 2-9-093 | N-Biph | N-Biph | methyl | methyl | methyl | methyl | methyl |
| 2-9-094 | N-Biph | N-Biph | b1 | H | b1 | H | H |
| 2-9-095 | N-Biph | N-Biph | b2 | H | b2 | H | H |
| 2-9-096 | N-Biph | N-Biph | b3 | H | b3 | H | H |
| 2-9-097 | N-Biph | N-Biph | H | H | H | H | a1 |
| 2-9-098 | N-Biph | N-Biph | t-bu | t-bu | t-bu | t-bu | a1 |
| 2-9-099 | N-Biph | N-Biph | methyl | methyl | methyl | methyl | a1 |
| 2-9-100 | N-Biph | N-Biph | b1 | b1 | b1 | b1 | a1 |
| 2-9-101 | N-Biph | N-Biph | b2 | b2 | b2 | b2 | a1 |
| 2-9-102 | N-Biph | N-Biph | b3 | b3 | b3 | b3 | a1 |
| 2-9-103 | N-Biph | N-Biph | H | H | H | H | a2 |
| 2-9-104 | N-Biph | N-Biph | t-bu | t-bu | t-bu | t-bu | a2 |
| 2-9-105 | N-Biph | N-Biph | methyl | methyl | methyl | methyl | a2 |
| 2-9-106 | N-Biph | N-Biph | b1 | b1 | b1 | b1 | a2 |
| 2-9-107 | N-Biph | N-Biph | b2 | b2 | b2 | b2 | a2 |
| 2-9-108 | N-Biph | N-Biph | b3 | b3 | b3 | b3 | a2 |
| 2-9-109 | N-Biph | N-Biph | H | H | H | H | a3 |
| 2-9-110 | N-Biph | N-Biph | t-bu | t-bu | t-bu | t-bu | a3 |
| 2-9-111 | N-Biph | N-Biph | methyl | methyl | methyl | methyl | a3 |
| 2-9-112 | N-Biph | N-Biph | b1 | b1 | b1 | b1 | a3 |
| 2-9-113 | N-Biph | N-Biph | b2 | b2 | b2 | b2 | a3 |
| 2-9-114 | N-Biph | N-Biph | b3 | b3 | b3 | b3 | a3 |
| 2-9-115 | N-Biph | N-Biph | H | H | H | H | a4 |
| 2-9-116 | N-Biph | N-Biph | t-bu | t-bu | t-bu | t-bu | a4 |
| 2-9-117 | N-Biph | N-Biph | methyl | methyl | methyl | methyl | a4 |
| 2-9-118 | N-Biph | N-Biph | b1 | b1 | b1 | b1 | a4 |
| 2-9-119 | N-Biph | N-Biph | b2 | b2 | b2 | b2 | a4 |
| 2-9-120 | N-Biph | N-Biph | b3 | b3 | b3 | b3 | a4 |
| 2-9-121 | N-Biph | N-Biph | H | H | H | H | a5 |
| 2-9-122 | N-Biph | N-Biph | t-bu | t-bu | t-bu | t-bu | a5 |
| 2-9-123 | N-Biph | N-Biph | methyl | methyl | methyl | methyl | a5 |
| 2-9-124 | N-Biph | N-Biph | b1 | b1 | b1 | b1 | a5 |
| 2-9-125 | N-Biph | N-Biph | b2 | b2 | b2 | b2 | a5 |
| 2-9-126 | N-Biph | N-Biph | b3 | b3 | b3 | b3 | a5 |
| 2-9-127 | N-Biph | N-Biph | H | H | H | H | a6 |
| 2-9-128 | N-Biph | N-Biph | t-bu | t-bu | t-bu | t-bu | a6 |
| 2-9-129 | N-Biph | N-Biph | methyl | methyl | methyl | methyl | a6 |
| 2-9-130 | N-Biph | N-Biph | b1 | b1 | b1 | b1 | a6 |
| 2-9-131 | N-Biph | N-Biph | b2 | b2 | b2 | b2 | a6 |
| 2-9-132 | N-Biph | N-Biph | b3 | b3 | b3 | b3 | a6 |
| 2-9-133 | O | N—Ph | H | H | H | H | H |
| 2-9-134 | O | N—Ph | t-bu | H | t-bu | H | H |
| 2-9-135 | O | N—Ph | methyl | H | methyl | H | H |
| 2-9-136 | O | N—Ph | t-bu | t-bu | t-bu | t-bu | t-bu |
| 2-9-137 | O | N—Ph | methyl | methyl | methyl | methyl | methyl |
| 2-9-138 | O | N—Ph | b1 | H | b1 | H | H |
| 2-9-139 | O | N—Ph | b2 | H | b2 | H | H |
| 2-9-140 | O | N—Ph | b3 | H | b3 | H | H |
| 2-9-141 | O | N—Ph | H | H | H | H | a1 |
| 2-9-142 | O | N—Ph | t-bu | t-bu | t-bu | t-bu | a1 |
| 2-9-143 | O | N—Ph | methyl | methyl | methyl | methyl | a1 |
| 2-9-144 | O | N—Ph | b1 | b1 | b1 | b1 | a1 |
| 2-9-145 | O | N—Ph | b2 | b2 | b2 | b2 | a1 |
| 2-9-146 | O | N—Ph | b3 | b3 | b3 | b3 | a1 |
| 2-9-147 | O | N—Ph | H | H | H | H | a2 |
| 2-9-148 | O | N—Ph | t-bu | t-bu | t-bu | t-bu | a2 |
| 2-9-149 | O | N—Ph | methyl | methyl | methyl | methyl | a2 |
| 2-9-150 | O | N—Ph | b1 | b1 | b1 | b1 | a2 |
| 2-9-151 | O | N—Ph | b2 | b2 | b2 | b2 | a2 |
| 2-9-152 | O | N—Ph | b3 | b3 | b3 | b3 | a2 |
| 2-9-153 | O | N—Ph | H | H | H | H | a3 |
| 2-9-154 | O | N—Ph | t-bu | t-bu | t-bu | t-bu | a3 |
| 2-9-155 | O | N—Ph | methyl | methyl | methyl | methyl | a3 |
| 2-9-156 | O | N—Ph | b1 | b1 | b1 | b1 | a3 |
| 2-9-157 | O | N—Ph | b2 | b2 | b2 | b2 | a3 |
| 2-9-158 | O | N—Ph | b3 | b3 | b3 | b3 | a3 |
| 2-9-159 | O | N—Ph | H | H | H | H | a4 |
| 2-9-160 | O | N—Ph | t-bu | t-bu | t-bu | t-bu | a4 |
| 2-9-161 | O | N—Ph | methyl | methyl | methyl | methyl | a4 |
| 2-9-162 | O | N—Ph | b1 | b1 | b1 | b1 | a4 |
| 2-9-163 | O | N—Ph | b2 | b2 | b2 | b2 | a4 |
| 2-9-164 | O | N—Ph | b3 | b3 | b3 | b3 | a4 |
| 2-9-165 | O | N—Ph | H | H | H | H | a5 |
| 2-9-166 | O | N—Ph | t-bu | t-bu | t-bu | t-bu | a5 |
| 2-9-167 | O | N—Ph | methyl | methyl | methyl | methyl | a5 |
| 2-9-168 | O | N—Ph | b1 | b1 | b1 | b1 | a5 |
| 2-9-169 | O | N—Ph | b2 | b2 | b2 | b2 | a5 |
| 2-9-170 | O | N—Ph | b3 | b3 | b3 | b3 | a5 |
| 2-9-171 | O | N—Ph | H | H | H | H | a6 |
| 2-9-172 | O | N—Ph | t-bu | t-bu | t-bu | t-bu | a6 |
| 2-9-173 | O | N—Ph | methyl | methyl | methyl | methyl | a6 |
| 2-9-174 | O | N—Ph | b1 | b1 | b1 | b1 | a6 |
| 2-9-175 | O | N—Ph | b2 | b2 | b2 | b2 | a6 |
| 2-9-176 | O | N—Ph | b3 | b3 | b3 | b3 | a6 |
| 2-9-177 | S | N—Ph | H | H | H | H | H |
| 2-9-178 | S | N—Ph | t-bu | H | t-bu | H | H |
| 2-9-179 | S | N—Ph | methyl | H | methyl | H | H |
| 2-9-180 | S | N—Ph | t-bu | t-bu | t-bu | t-bu | t-bu |
| 2-9-181 | S | N—Ph | methyl | methyl | methyl | methyl | methyl |
| 2-9-182 | S | N—Ph | b1 | H | b1 | H | H |
| 2-9-183 | S | N—Ph | b2 | H | b2 | H | H |
| 2-9-184 | S | N—Ph | b3 | H | b3 | H | H |
| 2-9-185 | S | N—Ph | H | H | H | H | a1 |
| 2-9-186 | S | N—Ph | t-bu | t-bu | t-bu | t-bu | a1 |
| 2-9-187 | S | N—Ph | methyl | methyl | methyl | methyl | a1 |
| 2-9-188 | S | N—Ph | b1 | b1 | b1 | b1 | a1 |
| 2-9-189 | S | N—Ph | b2 | b2 | b2 | b2 | a1 |
| 2-9-190 | S | N—Ph | b3 | b3 | b3 | b3 | a1 |
| 2-9-191 | S | N—Ph | H | H | H | H | a2 |
| 2-9-192 | S | N—Ph | t-bu | t-bu | t-bu | t-bu | a2 |
| 2-9-193 | S | N—Ph | methyl | methyl | methyl | methyl | a2 |
| 2-9-194 | S | N—Ph | b1 | b1 | b1 | b1 | a2 |
| 2-9-195 | S | N—Ph | b2 | b2 | b2 | b2 | a2 |
| 2-9-196 | S | N—Ph | b3 | b3 | b3 | b3 | a2 |
| 2-9-197 | S | N—Ph | H | H | H | H | a3 |
| 2-9-198 | S | N—Ph | t-bu | t-bu | t-bu | t-bu | a3 |
| 2-9-199 | S | N—Ph | methyl | methyl | methyl | methyl | a3 |
| 2-9-200 | S | N—Ph | b1 | b1 | b1 | b1 | a3 |
| 2-9-201 | S | N—Ph | b2 | b2 | b2 | b2 | a3 |
| 2-9-202 | S | N—Ph | b3 | b3 | b3 | b3 | a3 |
| 2-9-203 | S | N—Ph | H | H | H | H | a4 |
| 2-9-204 | S | N—Ph | t-bu | t-bu | t-bu | t-bu | a4 |
| 2-9-205 | S | N—Ph | methyl | methyl | methyl | methyl | a4 |
| 2-9-206 | S | N—Ph | b1 | b1 | b1 | b1 | a4 |
| 2-9-207 | S | N—Ph | b2 | b2 | b2 | b2 | a4 |
| 2-9-208 | S | N—Ph | b3 | b3 | b3 | b3 | a4 |
| 2-9-209 | S | N—Ph | H | H | H | H | a5 |
| 2-9-210 | S | N—Ph | t-bu | t-bu | t-bu | t-bu | a5 |
| 2-9-211 | S | N—Ph | methyl | methyl | methyl | methyl | a5 |
| 2-9-212 | S | N—Ph | b1 | b1 | b1 | b1 | a5 |
| 2-9-213 | S | N—Ph | b2 | b2 | b2 | b2 | a5 |
| 2-9-214 | S | N—Ph | b3 | b3 | b3 | b3 | a5 |
| 2-9-215 | S | N—Ph | H | H | H | H | a6 |
| 2-9-216 | S | N—Ph | t-bu | t-bu | t-bu | t-bu | a6 |
| 2-9-217 | S | N—Ph | methyl | methyl | methyl | methyl | a6 |
| 2-9-218 | S | N—Ph | b1 | b1 | b1 | b1 | a6 |
| 2-9-219 | S | N—Ph | b2 | b2 | b2 | b2 | a6 |
| 2-9-220 | S | N—Ph | b3 | b3 | b3 | b3 | a6 |

### Group 2-10

The compounds are represented by the formula 2-10 below, wherein CY₂ is a benzene group, X₁ is B, and Y1, Y2, R1b, R1d, R3a, (R2)a2 are each shown in the table 10 below,

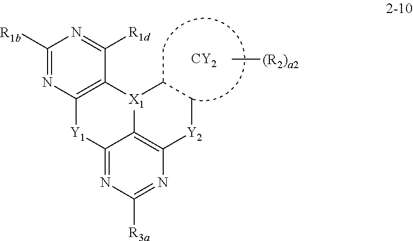

TABLE 10

| Compound | | | | | | |
|---|---|---|---|---|---|---|
| No. | Y1 | Y2 | R1b | R1d | R3a | (R2)a2 |
| 2-10-001 | N—Ph | N—Ph | H | H | H | H |
| 2-10-002 | N—Ph | N—Ph | t-bu | H | t-bu | H |
| 2-10-003 | N—Ph | N—Ph | methyl | H | methyl | H |
| 2-10-004 | N—Ph | N—Ph | t-bu | t-bu | t-bu | t-bu |
| 2-10-005 | N—Ph | N—Ph | methyl | methyl | methyl | methyl |
| 2-10-006 | N—Ph | N—Ph | b1 | H | b1 | H |
| 2-10-007 | N—Ph | N—Ph | b2 | H | b2 | H |
| 2-10-008 | N—Ph | N—Ph | b3 | H | b3 | H |
| 2-10-009 | N—Ph | N—Ph | H | H | H | a1 |
| 2-10-010 | N—Ph | N—Ph | t-bu | H | t-bu | a1 |
| 2-10-011 | N—Ph | N—Ph | methyl | H | methyl | a1 |
| 2-10-012 | N—Ph | N—Ph | b1 | H | b1 | a1 |
| 2-10-013 | N—Ph | N—Ph | b2 | H | b2 | a1 |
| 2-10-014 | N—Ph | N—Ph | b3 | H | b3 | a1 |
| 2-10-015 | N—Ph | N—Ph | H | H | H | a2 |
| 2-10-016 | N—Ph | N—Ph | t-bu | H | t-bu | a2 |
| 2-10-017 | N—Ph | N—Ph | methyl | H | methyl | a2 |
| 2-10-018 | N—Ph | N—Ph | b1 | H | b1 | a2 |
| 2-10-019 | N—Ph | N—Ph | b2 | H | b2 | a2 |
| 2-10-020 | N—Ph | N—Ph | b3 | H | b3 | a2 |
| 2-10-021 | N—Ph | N—Ph | H | H | H | a3 |
| 2-10-022 | N—Ph | N—Ph | t-bu | H | t-bu | a3 |
| 2-10-023 | N—Ph | N—Ph | methyl | H | methyl | a3 |
| 2-10-024 | N—Ph | N—Ph | b1 | H | b1 | a3 |
| 2-10-025 | N—Ph | N—Ph | b2 | H | b2 | a3 |
| 2-10-026 | N—Ph | N—Ph | b3 | H | b3 | a3 |
| 2-10-027 | N—Ph | N—Ph | H | H | H | a4 |
| 2-10-028 | N—Ph | N—Ph | t-bu | H | t-bu | a4 |
| 2-10-029 | N—Ph | N—Ph | methyl | H | methyl | a4 |
| 2-10-030 | N—Ph | N—Ph | b1 | H | b1 | a4 |
| 2-10-031 | N—Ph | N—Ph | b2 | H | b2 | a4 |
| 2-10-032 | N—Ph | N—Ph | b3 | H | b3 | a4 |
| 2-10-033 | N—Ph | N—Ph | H | H | H | a5 |
| 2-10-034 | N—Ph | N—Ph | t-bu | H | t-bu | a5 |
| 2-10-035 | N—Ph | N—Ph | methyl | H | methyl | a5 |
| 2-10-036 | N—Ph | N—Ph | b1 | H | b1 | a5 |
| 2-10-037 | N—Ph | N—Ph | b2 | H | b2 | a5 |
| 2-10-038 | N—Ph | N—Ph | b3 | H | b3 | a5 |
| 2-10-039 | N—Ph | N—Ph | H | H | H | a6 |
| 2-10-040 | N—Ph | N—Ph | t-bu | H | t-bu | a6 |
| 2-10-041 | N—Ph | N—Ph | methyl | H | methyl | a6 |
| 2-10-042 | N—Ph | N—Ph | b1 | H | b1 | a6 |
| 2-10-043 | N—Ph | N—Ph | b2 | H | b2 | a6 |
| 2-10-044 | N—Ph | N—Ph | b3 | H | b3 | a6 |
| 2-10-045 | N—Ph | N—Ph | H | H | H | b1 |
| 2-10-046 | N—Ph | N—Ph | t-bu | H | t-bu | b1 |
| 2-10-047 | N—Ph | N—Ph | methyl | H | methyl | b1 |
| 2-10-048 | N—Ph | N—Ph | b1 | H | b1 | b1 |
| 2-10-049 | N—Ph | N—Ph | b2 | H | b2 | b1 |
| 2-10-050 | N—Ph | N—Ph | b3 | H | b3 | b1 |
| 2-10-051 | N—Ph | N—Ph | H | H | H | b2 |
| 2-10-052 | N—Ph | N—Ph | t-bu | H | t-bu | b2 |
| 2-10-053 | N—Ph | N—Ph | methyl | H | methyl | b2 |
| 2-10-054 | N—Ph | N—Ph | b1 | H | b1 | b2 |
| 2-10-055 | N—Ph | N—Ph | b2 | H | b2 | b2 |
| 2-10-056 | N—Ph | N—Ph | b3 | H | b3 | b2 |
| 2-10-057 | N—Ph | N—Ph | H | H | H | b3 |
| 2-10-058 | N—Ph | N—Ph | t-bu | H | t-bu | b3 |
| 2-10-059 | N—Ph | N—Ph | methyl | H | methyl | b3 |
| 2-10-060 | N—Ph | N—Ph | b1 | H | b1 | b3 |
| 2-10-061 | N—Ph | N—Ph | b2 | H | b2 | b3 |
| 2-10-062 | N—Ph | N—Ph | b3 | H | b3 | b3 |
| 2-10-063 | N—Ph | N—Ph | H | H | H | b4 |
| 2-10-064 | N—Ph | N—Ph | t-bu | H | t-bu | b4 |
| 2-10-065 | N—Ph | N—Ph | methyl | H | methyl | b4 |
| 2-10-066 | N—Ph | N—Ph | b1 | H | b1 | b4 |
| 2-10-067 | N—Ph | N—Ph | b2 | H | b2 | b4 |
| 2-10-068 | N—Ph | N—Ph | b3 | H | b3 | b4 |
| 2-10-069 | N—Ph | N—Ph | H | H | H | b5 |
| 2-10-070 | N—Ph | N—Ph | t-bu | H | t-bu | b5 |
| 2-10-071 | N—Ph | N—Ph | methyl | H | methyl | b5 |
| 2-10-072 | N—Ph | N—Ph | b1 | H | b1 | b5 |
| 2-10-073 | N—Ph | N—Ph | b2 | H | b2 | b5 |
| 2-10-074 | N—Ph | N—Ph | b3 | H | b3 | b5 |
| 2-10-075 | N—Ph | N—Ph | H | H | H | b6 |
| 2-10-076 | N—Ph | N—Ph | t-bu | H | t-bu | b6 |
| 2-10-077 | N—Ph | N—Ph | methyl | H | methyl | b6 |
| 2-10-078 | N—Ph | N—Ph | b1 | H | b1 | b6 |
| 2-10-079 | N—Ph | N—Ph | b2 | H | b2 | b6 |
| 2-10-080 | N—Ph | N—Ph | b3 | H | b3 | b6 |
| 2-10-081 | N—Ph | N—Ph | H | H | H | b7 |
| 2-10-082 | N—Ph | N—Ph | t-bu | H | t-bu | b7 |
| 2-10-083 | N—Ph | N—Ph | methyl | H | methyl | b7 |
| 2-10-084 | N—Ph | N—Ph | b1 | H | b1 | b7 |
| 2-10-085 | N—Ph | N—Ph | b2 | H | b2 | b7 |
| 2-10-086 | N—Ph | N—Ph | b3 | H | b3 | b7 |
| 2-10-087 | N—Ph | N—Ph | H | H | H | b8 |
| 2-10-088 | N—Ph | N—Ph | t-bu | H | t-bu | b8 |
| 2-10-089 | N—Ph | N—Ph | methyl | H | methyl | b8 |
| 2-10-090 | N—Ph | N—Ph | b1 | H | b1 | b8 |
| 2-10-091 | N—Ph | N—Ph | b2 | H | b2 | b8 |
| 2-10-092 | N—Ph | N—Ph | b3 | H | b3 | b8 |
| 2-10-093 | N—Ph | N—Ph | H | H | H | b9 |
| 2-10-094 | N—Ph | N—Ph | t-bu | H | t-bu | b9 |
| 2-10-095 | N—Ph | N—Ph | methyl | H | methyl | b9 |
| 2-10-096 | N—Ph | N—Ph | b1 | H | b1 | b9 |
| 2-10-097 | N—Ph | N—Ph | b2 | H | b2 | b9 |
| 2-10-098 | N—Ph | N—Ph | b3 | H | b3 | b9 |
| 2-10-099 | N—Ph | N—Ph | H | H | H | b10 |
| 2-10-100 | N—Ph | N—Ph | t-bu | H | t-bu | b10 |
| 2-10-101 | N—Ph | N—Ph | methyl | H | methyl | b10 |
| 2-10-102 | N—Ph | N—Ph | b1 | H | b1 | b10 |
| 2-10-103 | N—Ph | N—Ph | b2 | H | b2 | b10 |
| 2-10-104 | N—Ph | N—Ph | b3 | H | b3 | b10 |
| 2-10-105 | N-tolyl | N-tolyl | H | H | H | H |
| 2-10-106 | N-tolyl | N-tolyl | t-bu | H | t-bu | H |
| 2-10-107 | N-tolyl | N-tolyl | methyl | H | methyl | H |
| 2-10-108 | N-tolyl | N-tolyl | t-bu | t-bu | t-bu | t-bu |
| 2-10-109 | N-tolyl | N-tolyl | methyl | methyl | methyl | methyl |
| 2-10-110 | N-tolyl | N-tolyl | b1 | H | b1 | H |
| 2-10-111 | N-tolyl | N-tolyl | b2 | H | b2 | H |
| 2-10-112 | N-tolyl | N-tolyl | b3 | H | b3 | H |
| 2-10-113 | N-tolyl | N-tolyl | H | H | H | a1 |
| 2-10-114 | N-tolyl | N-tolyl | t-bu | H | t-bu | a1 |
| 2-10-115 | N-tolyl | N-tolyl | methyl | H | methyl | a1 |
| 2-10-116 | N-tolyl | N-tolyl | b1 | H | b1 | a1 |
| 2-10-117 | N-tolyl | N-tolyl | b2 | H | b2 | a1 |
| 2-10-118 | N-tolyl | N-tolyl | b3 | H | b3 | a1 |
| 2-10-119 | N-tolyl | N-tolyl | H | H | H | a2 |
| 2-10-120 | N-tolyl | N-tolyl | t-bu | H | t-bu | a2 |
| 2-10-121 | N-tolyl | N-tolyl | methyl | H | methyl | a2 |
| 2-10-122 | N-tolyl | N-tolyl | b1 | H | b1 | a2 |
| 2-10-123 | N-tolyl | N-tolyl | b2 | H | b2 | a2 |
| 2-10-124 | N-tolyl | N-tolyl | b3 | H | b3 | a2 |
| 2-10-125 | N-tolyl | N-tolyl | H | H | H | a3 |
| 2-10-126 | N-tolyl | N-tolyl | t-bu | H | t-bu | a3 |
| 2-10-127 | N-tolyl | N-tolyl | methyl | H | methyl | a3 |
| 2-10-128 | N-tolyl | N-tolyl | b1 | H | b1 | a3 |
| 2-10-129 | N-tolyl | N-tolyl | b2 | H | b2 | a3 |
| 2-10-130 | N-tolyl | N-tolyl | b3 | H | b3 | a3 |
| 2-10-131 | N-tolyl | N-tolyl | H | H | H | a4 |
| 2-10-132 | N-tolyl | N-tolyl | t-bu | H | t-bu | a4 |
| 2-10-133 | N-tolyl | N-tolyl | methyl | H | methyl | a4 |
| 2-10-134 | N-tolyl | N-tolyl | b1 | H | b1 | a4 |
| 2-10-135 | N-tolyl | N-tolyl | b2 | H | b2 | a4 |
| 2-10-136 | N-tolyl | N-tolyl | b3 | H | b3 | a4 |
| 2-10-137 | N-tolyl | N-tolyl | H | H | H | a5 |
| 2-10-138 | N-tolyl | N-tolyl | t-bu | H | t-bu | a5 |
| 2-10-139 | N-tolyl | N-tolyl | methyl | H | methyl | a5 |
| 2-10-140 | N-tolyl | N-tolyl | b1 | H | b1 | a5 |
| 2-10-141 | N-tolyl | N-tolyl | b2 | H | b2 | a5 |
| 2-10-142 | N-tolyl | N-tolyl | b3 | H | b3 | a5 |
| 2-10-143 | N-tolyl | N-tolyl | H | H | H | a6 |
| 2-10-144 | N-tolyl | N-tolyl | t-bu | H | t-bu | a6 |
| 2-10-145 | N-tolyl | N-tolyl | methyl | H | methyl | a6 |
| 2-10-146 | N-tolyl | N-tolyl | b1 | H | b1 | a6 |
| 2-10-147 | N-tolyl | N-tolyl | b2 | H | b2 | a6 |
| 2-10-148 | N-tolyl | N-tolyl | b3 | H | b3 | a6 |
| 2-10-149 | N-tolyl | N-tolyl | H | H | H | b1 |
| 2-10-150 | N-tolyl | N-tolyl | t-bu | H | t-bu | b1 |
| 2-10-151 | N-tolyl | N-tolyl | methyl | H | methyl | b1 |
| 2-10-152 | N-tolyl | N-tolyl | b1 | H | b1 | b1 |
| 2-10-153 | N-tolyl | N-tolyl | b2 | H | b2 | b1 |
| 2-10-154 | N-tolyl | N-tolyl | b3 | H | b3 | b1 |
| 2-10-155 | N-tolyl | N-tolyl | H | H | H | b2 |
| 2-10-156 | N-tolyl | N-tolyl | t-bu | H | t-bu | b2 |
| 2-10-157 | N-tolyl | N-tolyl | methyl | H | methyl | b2 |
| 2-10-158 | N-tolyl | N-tolyl | b1 | H | b1 | b2 |
| 2-10-159 | N-tolyl | N-tolyl | b2 | H | b2 | b2 |
| 2-10-160 | N-tolyl | N-tolyl | b3 | H | b3 | b2 |
| 2-10-161 | N-tolyl | N-tolyl | H | H | H | b3 |
| 2-10-162 | N-tolyl | N-tolyl | t-bu | H | t-bu | b3 |
| 2-10-163 | N-tolyl | N-tolyl | methyl | H | methyl | b3 |
| 2-10-164 | N-tolyl | N-tolyl | b1 | H | b1 | b3 |
| 2-10-165 | N-tolyl | N-tolyl | b2 | H | b2 | b3 |
| 2-10-166 | N-tolyl | N-tolyl | b3 | H | b3 | b3 |
| 2-10-167 | N-tolyl | N-tolyl | H | H | H | b4 |
| 2-10-168 | N-tolyl | N-tolyl | t-bu | H | t-bu | b4 |
| 2-10-169 | N-tolyl | N-tolyl | methyl | H | methyl | b4 |
| 2-10-170 | N-tolyl | N-tolyl | b1 | H | b1 | b4 |
| 2-10-171 | N-tolyl | N-tolyl | b2 | H | b2 | b4 |
| 2-10-172 | N-tolyl | N-tolyl | b3 | H | b3 | b4 |
| 2-10-173 | N-tolyl | N-tolyl | H | H | H | b5 |
| 2-10-174 | N-tolyl | N-tolyl | t-bu | H | t-bu | b5 |
| 2-10-175 | N-tolyl | N-tolyl | methyl | H | methyl | b5 |
| 2-10-176 | N-tolyl | N-tolyl | b1 | H | b1 | b5 |
| 2-10-177 | N-tolyl | N-tolyl | b2 | H | b2 | b5 |
| 2-10-178 | N-tolyl | N-tolyl | b3 | H | b3 | b5 |
| 2-10-179 | N-tolyl | N-tolyl | H | H | H | b6 |
| 2-10-180 | N-tolyl | N-tolyl | t-bu | H | t-bu | b6 |
| 2-10-181 | N-tolyl | N-tolyl | methyl | H | methyl | b6 |
| 2-10-182 | N-tolyl | N-tolyl | b1 | H | b1 | b6 |
| 2-10-183 | N-tolyl | N-tolyl | b2 | H | b2 | b6 |
| 2-10-184 | N-tolyl | N-tolyl | b3 | H | b3 | b6 |
| 2-10-185 | N-tolyl | N-tolyl | H | H | H | b7 |
| 2-10-186 | N-tolyl | N-tolyl | t-bu | H | t-bu | b7 |
| 2-10-187 | N-tolyl | N-tolyl | methyl | H | methyl | b7 |
| 2-10-188 | N-tolyl | N-tolyl | b1 | H | b1 | b7 |
| 2-10-189 | N-tolyl | N-tolyl | b2 | H | b2 | b7 |
| 2-10-190 | N-tolyl | N-tolyl | b3 | H | b3 | b7 |
| 2-10-191 | N-tolyl | N-tolyl | H | H | H | b8 |
| 2-10-192 | N-tolyl | N-tolyl | t-bu | H | t-bu | b8 |
| 2-10-193 | N-tolyl | N-tolyl | methyl | H | methyl | b8 |
| 2-10-194 | N-tolyl | N-tolyl | b1 | H | b1 | b8 |
| 2-10-195 | N-tolyl | N-tolyl | b2 | H | b2 | b8 |
| 2-10-196 | N-tolyl | N-tolyl | b3 | H | b3 | b8 |
| 2-10-197 | N-tolyl | N-tolyl | H | H | H | b9 |
| 2-10-198 | N-tolyl | N-tolyl | t-bu | H | t-bu | b9 |
| 2-10-199 | N-tolyl | N-tolyl | methyl | H | methyl | b9 |
| 2-10-200 | N-tolyl | N-tolyl | b1 | H | b1 | b9 |
| 2-10-201 | N-tolyl | N-tolyl | b2 | H | b2 | b9 |
| 2-10-202 | N-tolyl | N-tolyl | b3 | H | b3 | b9 |
| 2-10-203 | N-tolyl | N-tolyl | H | H | H | b10 |
| 2-10-204 | N-tolyl | N-tolyl | t-bu | H | t-bu | b10 |
| 2-10-205 | N-tolyl | N-tolyl | methyl | H | methyl | b10 |
| 2-10-206 | N-tolyl | N-tolyl | b1 | H | b1 | b10 |
| 2-10-207 | N-tolyl | N-tolyl | b2 | H | b2 | b10 |
| 2-10-208 | N-tolyl | N-tolyl | b3 | H | b3 | b10 |
| 2-10-209 | N-Biph | N-Biph | H | H | H | H |
| 2-10-210 | N-Biph | N-Biph | t-bu | H | t-bu | H |
| 2-10-211 | N-Biph | N-Biph | methyl | H | methyl | H |
| 2-10-212 | N-Biph | N-Biph | t-bu | t-bu | t-bu | t-bu |
| 2-10-213 | N-Biph | N-Biph | methyl | methyl | methyl | methyl |
| 2-10-214 | N-Biph | N-Biph | b1 | H | b1 | H |
| 2-10-215 | N-Biph | N-Biph | b2 | H | b2 | H |
| 2-10-216 | N-Biph | N-Biph | b3 | H | b3 | H |
| 2-10-217 | N-Biph | N-Biph | H | H | H | a1 |
| 2-10-218 | N-Biph | N-Biph | t-bu | H | t-bu | a1 |
| 2-10-219 | N-Biph | N-Biph | methyl | H | methyl | a1 |
| 2-10-220 | N-Biph | N-Biph | b1 | H | b1 | a1 |
| 2-10-221 | N-Biph | N-Biph | b2 | H | b2 | a1 |
| 2-10-222 | N-Biph | N-Biph | b3 | H | b3 | a1 |
| 2-10-223 | N-Biph | N-Biph | H | H | H | a2 |
| 2-10-224 | N-Biph | N-Biph | t-bu | H | t-bu | a2 |
| 2-10-225 | N-Biph | N-Biph | methyl | H | methyl | a2 |
| 2-10-226 | N-Biph | N-Biph | b1 | H | b1 | a2 |
| 2-10-227 | N-Biph | N-Biph | b2 | H | b2 | a2 |
| 2-10-228 | N-Biph | N-Biph | b3 | H | b3 | a2 |
| 2-10-229 | N-Biph | N-Biph | H | H | H | a3 |
| 2-10-230 | N-Biph | N-Biph | t-bu | H | t-bu | a3 |
| 2-10-231 | N-Biph | N-Biph | methyl | H | methyl | a3 |
| 2-10-232 | N-Biph | N-Biph | b1 | H | b1 | a3 |
| 2-10-233 | N-Biph | N-Biph | b2 | H | b2 | a3 |
| 2-10-234 | N-Biph | N-Biph | b3 | H | b3 | a3 |
| 2-10-235 | N-Biph | N-Biph | H | H | H | a4 |
| 2-10-236 | N-Biph | N-Biph | t-bu | H | t-bu | a4 |
| 2-10-237 | N-Biph | N-Biph | methyl | H | methyl | a4 |
| 2-10-238 | N-Biph | N-Biph | b1 | H | b1 | a4 |
| 2-10-239 | N-Biph | N-Biph | b2 | H | b2 | a4 |
| 2-10-240 | N-Biph | N-Biph | b3 | H | b3 | a4 |
| 2-10-241 | N-Biph | N-Biph | H | H | H | a5 |
| 2-10-242 | N-Biph | N-Biph | t-bu | H | t-bu | a5 |
| 2-10-243 | N-Biph | N-Biph | methyl | H | methyl | a5 |
| 2-10-244 | N-Biph | N-Biph | b1 | H | b1 | a5 |
| 2-10-245 | N-Biph | N-Biph | b2 | H | b2 | a5 |
| 2-10-246 | N-Biph | N-Biph | b3 | H | b3 | a5 |
| 2-10-247 | N-Biph | N-Biph | H | H | H | a6 |
| 2-10-248 | N-Biph | N-Biph | t-bu | H | t-bu | a6 |
| 2-10-249 | N-Biph | N-Biph | methyl | H | methyl | a6 |
| 2-10-250 | N-Biph | N-Biph | b1 | H | b1 | a6 |
| 2-10-251 | N-Biph | N-Biph | b2 | H | b2 | a6 |
| 2-10-252 | N-Biph | N-Biph | b3 | H | b3 | a6 |
| 2-10-253 | N-Biph | N-Biph | H | H | H | b1 |
| 2-10-254 | N-Biph | N-Biph | t-bu | H | t-bu | b1 |
| 2-10-255 | N-Biph | N-Biph | methyl | H | methyl | b1 |
| 2-10-256 | N-Biph | N-Biph | b1 | H | b1 | b1 |
| 2-10-257 | N-Biph | N-Biph | b2 | H | b2 | b1 |
| 2-10-258 | N-Biph | N-Biph | b3 | H | b3 | b1 |
| 2-10-259 | N-Biph | N-Biph | H | H | H | b2 |
| 2-10-260 | N-Biph | N-Biph | t-bu | H | t-bu | b2 |
| 2-10-261 | N-Biph | N-Biph | methyl | H | methyl | b2 |
| 2-10-262 | N-Biph | N-Biph | b1 | H | b1 | b2 |
| 2-10-263 | N-Biph | N-Biph | b2 | H | b2 | b2 |
| 2-10-264 | N-Biph | N-Biph | b3 | H | b3 | b2 |
| 2-10-265 | N-Biph | N-Biph | H | H | H | b3 |
| 2-10-266 | N-Biph | N-Biph | t-bu | H | t-bu | b3 |
| 2-10-267 | N-Biph | N-Biph | methyl | H | methyl | b3 |
| 2-10-268 | N-Biph | N-Biph | b1 | H | b1 | b3 |
| 2-10-269 | N-Biph | N-Biph | b2 | H | b2 | b3 |
| 2-10-270 | N-Biph | N-Biph | b3 | H | b3 | b3 |
| 2-10-271 | N-Biph | N-Biph | H | H | H | b4 |
| 2-10-272 | N-Biph | N-Biph | t-bu | H | t-bu | b4 |
| 2-10-273 | N-Biph | N-Biph | methyl | H | methyl | b4 |
| 2-10-274 | N-Biph | N-Biph | b1 | H | b1 | b4 |
| 2-10-275 | N-Biph | N-Biph | b2 | H | b2 | b4 |
| 2-10-276 | N-Biph | N-Biph | b3 | H | b3 | b4 |
| 2-10-277 | N-Biph | N-Biph | H | H | H | b5 |
| 2-10-278 | N-Biph | N-Biph | t-bu | H | t-bu | b5 |
| 2-10-279 | N-Biph | N-Biph | methyl | H | methyl | b5 |
| 2-10-280 | N-Biph | N-Biph | b1 | H | b1 | b5 |
| 2-10-281 | N-Biph | N-Biph | b2 | H | b2 | b5 |
| 2-10-282 | N-Biph | N-Biph | b3 | H | b3 | b5 |
| 2-10-283 | N-Biph | N-Biph | H | H | H | b6 |
| 2-10-284 | N-Biph | N-Biph | t-bu | H | t-bu | b6 |
| 2-10-285 | N-Biph | N-Biph | methyl | H | methyl | b6 |
| 2-10-286 | N-Biph | N-Biph | b1 | H | b1 | b6 |
| 2-10-287 | N-Biph | N-Biph | b2 | H | b2 | b6 |
| 2-10-288 | N-Biph | N-Biph | b3 | H | b3 | b6 |
| 2-10-289 | N-Biph | N-Biph | H | H | H | b7 |
| 2-10-290 | N-Biph | N-Biph | t-bu | H | t-bu | b7 |
| 2-10-291 | N-Biph | N-Biph | methyl | H | methyl | b7 |
| 2-10-292 | N-Biph | N-Biph | b1 | H | b1 | b7 |
| 2-10-293 | N-Biph | N-Biph | b2 | H | b2 | b7 |
| 2-10-294 | N-Biph | N-Biph | b3 | H | b3 | b7 |
| 2-10-295 | N-Biph | N-Biph | H | H | H | b8 |
| 2-10-296 | N-Biph | N-Biph | t-bu | H | t-bu | b8 |
| 2-10-297 | N-Biph | N-Biph | methyl | H | methyl | b8 |
| 2-10-298 | N-Biph | N-Biph | b1 | H | b1 | b8 |
| 2-10-299 | N-Biph | N-Biph | b2 | H | b2 | b8 |
| 2-10-300 | N-Biph | N-Biph | b3 | H | b3 | b8 |
| 2-10-301 | N-Biph | N-Biph | H | H | H | b9 |
| 2-10-302 | N-Biph | N-Biph | t-bu | H | t-bu | b9 |
| 2-10-303 | N-Biph | N-Biph | methyl | H | methyl | b9 |
| 2-10-304 | N-Biph | N-Biph | b1 | H | b1 | b9 |
| 2-10-305 | N-Biph | N-Biph | b2 | H | b2 | b9 |
| 2-10-306 | N-Biph | N-Biph | b3 | H | b3 | b9 |
| 2-10-307 | N-Biph | N-Biph | H | H | H | b10 |
| 2-10-308 | N-Biph | N-Biph | t-bu | H | t-bu | b10 |
| 2-10-309 | N-Biph | N-Biph | methyl | H | methyl | b10 |
| 2-10-310 | N-Biph | N-Biph | b1 | H | b1 | b10 |
| 2-10-311 | N-Biph | N-Biph | b2 | H | b2 | b10 |
| 2-10-312 | N-Biph | N-Biph | b3 | H | b3 | b10 |
| 2-10-313 | O | N—Ph | H | H | H | H |
| 2-10-314 | O | N—Ph | t-bu | H | t-bu | H |
| 2-10-315 | O | N—Ph | methyl | H | methyl | H |
| 2-10-316 | O | N—Ph | t-bu | t-bu | t-bu | t-bu |
| 2-10-317 | O | N—Ph | methyl | methyl | methyl | methyl |
| 2-10-318 | O | N—Ph | b1 | H | b1 | H |
| 2-10-319 | O | N—Ph | b2 | H | b2 | H |
| 2-10-320 | O | N—Ph | b3 | H | b3 | H |
| 2-10-321 | O | N—Ph | H | H | H | a1 |
| 2-10-322 | O | N—Ph | t-bu | H | t-bu | a1 |
| 2-10-323 | O | N—Ph | methyl | H | methyl | a1 |
| 2-10-324 | O | N—Ph | b1 | H | b1 | a1 |
| 2-10-325 | O | N—Ph | b2 | H | b2 | a1 |
| 2-10-326 | O | N—Ph | b3 | H | b3 | a1 |
| 2-10-327 | O | N—Ph | H | H | H | a2 |
| 2-10-328 | O | N—Ph | t-bu | H | t-bu | a2 |
| 2-10-329 | O | N—Ph | methyl | H | methyl | a2 |
| 2-10-330 | O | N—Ph | b1 | H | b1 | a2 |
| 2-10-331 | O | N—Ph | b2 | H | b2 | a2 |
| 2-10-332 | O | N—Ph | b3 | H | b3 | a2 |
| 2-10-333 | O | N—Ph | H | H | H | a3 |
| 2-10-334 | O | N—Ph | t-bu | H | t-bu | a3 |
| 2-10-335 | O | N—Ph | methyl | H | methyl | a3 |
| 2-10-336 | O | N—Ph | b1 | H | b1 | a3 |
| 2-10-337 | O | N—Ph | b2 | H | b2 | a3 |
| 2-10-338 | O | N—Ph | b3 | H | b3 | a3 |
| 2-10-339 | O | N—Ph | H | H | H | a4 |
| 2-10-340 | O | N—Ph | t-bu | H | t-bu | a4 |
| 2-10-341 | O | N—Ph | methyl | H | methyl | a4 |
| 2-10-342 | O | N—Ph | b1 | H | b1 | a4 |
| 2-10-343 | O | N—Ph | b2 | H | b2 | a4 |
| 2-10-344 | O | N—Ph | b3 | H | b3 | a4 |
| 2-10-345 | O | N—Ph | H | H | H | a5 |
| 2-10-346 | O | N—Ph | t-bu | H | t-bu | a5 |
| 2-10-347 | O | N—Ph | methyl | H | methyl | a5 |
| 2-10-348 | O | N—Ph | b1 | H | b1 | a5 |
| 2-10-349 | O | N—Ph | b2 | H | b2 | a5 |
| 2-10-350 | O | N—Ph | b3 | H | b3 | a5 |
| 2-10-351 | O | N—Ph | H | H | H | a6 |
| 2-10-352 | O | N—Ph | t-bu | H | t-bu | a6 |
| 2-10-353 | O | N—Ph | methyl | H | methyl | a6 |
| 2-10-354 | O | N—Ph | b1 | H | b1 | a6 |
| 2-10-355 | O | N—Ph | b2 | H | b2 | a6 |
| 2-10-356 | O | N—Ph | b3 | H | b3 | a6 |
| 2-10-357 | O | N—Ph | H | H | H | b1 |
| 2-10-358 | O | N—Ph | t-bu | H | t-bu | b1 |
| 2-10-359 | O | N—Ph | methyl | H | methyl | b1 |
| 2-10-360 | O | N—Ph | b1 | H | b1 | b1 |
| 2-10-361 | O | N—Ph | b2 | H | b2 | b1 |
| 2-10-362 | O | N—Ph | b3 | H | b3 | b1 |
| 2-10-363 | O | N—Ph | H | H | H | b2 |
| 2-10-364 | O | N—Ph | t-bu | H | t-bu | b2 |
| 2-10-365 | O | N—Ph | methyl | H | methyl | b2 |
| 2-10-366 | O | N—Ph | b1 | H | b1 | b2 |
| 2-10-367 | O | N—Ph | b2 | H | b2 | b2 |
| 2-10-368 | O | N—Ph | b3 | H | b3 | b2 |
| 2-10-369 | O | N—Ph | H | H | H | b3 |
| 2-10-370 | O | N—Ph | t-bu | H | t-bu | b3 |
| 2-10-371 | O | N—Ph | methyl | H | methyl | b3 |
| 2-10-372 | O | N—Ph | b1 | H | b1 | b3 |
| 2-10-373 | O | N—Ph | b2 | H | b2 | b3 |
| 2-10-374 | O | N—Ph | b3 | H | b3 | b3 |
| 2-10-375 | O | N—Ph | H | H | H | b4 |
| 2-10-376 | O | N—Ph | t-bu | H | t-bu | b4 |
| 2-10-377 | O | N—Ph | methyl | H | methyl | b4 |
| 2-10-378 | O | N—Ph | b1 | H | b1 | b4 |
| 2-10-379 | O | N—Ph | b2 | H | b2 | b4 |
| 2-10-380 | O | N—Ph | b3 | H | b3 | b4 |
| 2-10-381 | O | N—Ph | H | H | H | b5 |
| 2-10-382 | O | N—Ph | t-bu | H | t-bu | b5 |
| 2-10-383 | O | N—Ph | methyl | H | methyl | b5 |
| 2-10-384 | O | N—Ph | b1 | H | b1 | b5 |
| 2-10-385 | O | N—Ph | b2 | H | b2 | b5 |
| 2-10-386 | O | N—Ph | b3 | H | b3 | b5 |
| 2-10-387 | O | N—Ph | H | H | H | b6 |
| 2-10-388 | O | N—Ph | t-bu | H | t-bu | b6 |
| 2-10-389 | O | N—Ph | methyl | H | methyl | b6 |
| 2-10-390 | O | N—Ph | b1 | H | b1 | b6 |
| 2-10-391 | O | N—Ph | b2 | H | b2 | b6 |
| 2-10-392 | O | N—Ph | b3 | H | b3 | b6 |
| 2-10-393 | O | N—Ph | H | H | H | b7 |
| 2-10-394 | O | N—Ph | t-bu | H | t-bu | b7 |
| 2-10-395 | O | N—Ph | methyl | H | methyl | b7 |
| 2-10-396 | O | N—Ph | b1 | H | b1 | b7 |
| 2-10-397 | O | N—Ph | b2 | H | b2 | b7 |
| 2-10-398 | O | N—Ph | b3 | H | b3 | b7 |
| 2-10-399 | O | N—Ph | H | H | H | b8 |
| 2-10-400 | O | N—Ph | t-bu | H | t-bu | b8 |
| 2-10-401 | O | N—Ph | methyl | H | methyl | b8 |
| 2-10-402 | O | N—Ph | b1 | H | b1 | b8 |
| 2-10-403 | O | N—Ph | b2 | H | b2 | b8 |
| 2-10-404 | O | N—Ph | b3 | H | b3 | b8 |
| 2-10-405 | O | N—Ph | H | H | H | b9 |
| 2-10-406 | O | N—Ph | t-bu | H | t-bu | b9 |
| 2-10-407 | O | N—Ph | methyl | H | methyl | b9 |
| 2-10-408 | O | N—Ph | b1 | H | b1 | b9 |
| 2-10-409 | O | N—Ph | b2 | H | b2 | b9 |
| 2-10-410 | O | N—Ph | b3 | H | b3 | b9 |
| 2-10-411 | O | N—Ph | H | H | H | b10 |
| 2-10-412 | O | N—Ph | t-bu | H | t-bu | b10 |
| 2-10-413 | O | N—Ph | methyl | H | methyl | b10 |
| 2-10-414 | O | N—Ph | b1 | H | b1 | b10 |
| 2-10-415 | O | N—Ph | b2 | H | b2 | b10 |
| 2-10-416 | O | N—Ph | b3 | H | b3 | b10 |
| 2-10-417 | S | N—Ph | H | H | H | H |
| 2-10-418 | S | N—Ph | t-bu | H | t-bu | H |
| 2-10-419 | S | N—Ph | methyl | H | methyl | H |
| 2-10-420 | S | N—Ph | t-bu | t-bu | t-bu | t-bu |
| 2-10-421 | S | N—Ph | methyl | methyl | methyl | methyl |
| 2-10-422 | S | N—Ph | b1 | H | b1 | H |
| 2-10-423 | S | N—Ph | b2 | H | b2 | H |
| 2-10-424 | S | N—Ph | b3 | H | b3 | H |
| 2-10-425 | S | N—Ph | H | H | H | a1 |
| 2-10-426 | S | N—Ph | t-bu | H | t-bu | a1 |
| 2-10-427 | S | N—Ph | methyl | H | methyl | a1 |
| 2-10-428 | S | N—Ph | b1 | H | b1 | a1 |
| 2-10-429 | S | N—Ph | b2 | H | b2 | a1 |
| 2-10-430 | S | N—Ph | b3 | H | b3 | a1 |
| 2-10-431 | S | N—Ph | H | H | H | a2 |
| 2-10-432 | S | N—Ph | t-bu | H | t-bu | a2 |
| 2-10-433 | S | N—Ph | methyl | H | methyl | a2 |
| 2-10-434 | S | N—Ph | b1 | H | b1 | a2 |
| 2-10-435 | S | N—Ph | b2 | H | b2 | a2 |
| 2-10-436 | S | N—Ph | b3 | H | b3 | a2 |
| 2-10-437 | S | N—Ph | H | H | H | a3 |
| 2-10-438 | S | N—Ph | t-bu | H | t-bu | a3 |
| 2-10-439 | S | N—Ph | methyl | H | methyl | a3 |
| 2-10-440 | S | N—Ph | b1 | H | b1 | a3 |
| 2-10-441 | S | N—Ph | b2 | H | b2 | a3 |
| 2-10-442 | S | N—Ph | b3 | H | b3 | a3 |
| 2-10-443 | S | N—Ph | H | H | H | a4 |
| 2-10-444 | S | N—Ph | t-bu | H | t-bu | a4 |
| 2-10-445 | S | N—Ph | methyl | H | methyl | a4 |
| 2-10-446 | S | N—Ph | b1 | H | b1 | a4 |
| 2-10-447 | S | N—Ph | b2 | H | b2 | a4 |
| 2-10-448 | S | N—Ph | b3 | H | b3 | a4 |
| 2-10-449 | S | N—Ph | H | H | H | a5 |
| 2-10-450 | S | N—Ph | t-bu | H | t-bu | a5 |
| 2-10-451 | S | N—Ph | methyl | H | methyl | a5 |
| 2-10-452 | S | N—Ph | b1 | H | b1 | a5 |
| 2-10-453 | S | N—Ph | b2 | H | b2 | a5 |
| 2-10-454 | S | N—Ph | b3 | H | b3 | a5 |
| 2-10-455 | S | N—Ph | H | H | H | a6 |
| 2-10-456 | S | N—Ph | t-bu | H | t-bu | a6 |
| 2-10-457 | S | N—Ph | methyl | H | methyl | a6 |
| 2-10-458 | S | N—Ph | b1 | H | b1 | a6 |
| 2-10-459 | S | N—Ph | b2 | H | b2 | a6 |
| 2-10-460 | S | N—Ph | b3 | H | b3 | a6 |
| 2-10-461 | S | N—Ph | H | H | H | b1 |
| 2-10-462 | S | N—Ph | t-bu | H | t-bu | b1 |
| 2-10-463 | S | N—Ph | methyl | H | methyl | b1 |
| 2-10-464 | S | N—Ph | b1 | H | b1 | b1 |
| 2-10-465 | S | N—Ph | b2 | H | b2 | b1 |
| 2-10-466 | S | N—Ph | b3 | H | b3 | b1 |
| 2-10-467 | S | N—Ph | H | H | H | b2 |
| 2-10-468 | S | N—Ph | t-bu | H | t-bu | b2 |
| 2-10-469 | S | N—Ph | methyl | H | methyl | b2 |
| 2-10-470 | S | N—Ph | b1 | H | b1 | b2 |
| 2-10-471 | S | N—Ph | b2 | H | b2 | b2 |
| 2-10-472 | S | N—Ph | b3 | H | b3 | b2 |
| 2-10-473 | S | N—Ph | H | H | H | b3 |
| 2-10-474 | S | N—Ph | t-bu | H | t-bu | b3 |
| 2-10-475 | S | N—Ph | methyl | H | methyl | b3 |
| 2-10-476 | S | N—Ph | b1 | H | b1 | b3 |
| 2-10-477 | S | N—Ph | b2 | H | b2 | b3 |
| 2-10-478 | S | N—Ph | b3 | H | b3 | b3 |
| 2-10-479 | S | N—Ph | H | H | H | b4 |
| 2-10-480 | S | N—Ph | t-bu | H | t-bu | b4 |
| 2-10-481 | S | N—Ph | methyl | H | methyl | b4 |
| 2-10-482 | S | N—Ph | b1 | H | b1 | b4 |
| 2-10-483 | S | N—Ph | b2 | H | b2 | b4 |
| 2-10-484 | S | N—Ph | b3 | H | b3 | b4 |
| 2-10-485 | S | N—Ph | H | H | H | b5 |
| 2-10-486 | S | N—Ph | t-bu | H | t-bu | b5 |
| 2-10-487 | S | N—Ph | methyl | H | methyl | b5 |
| 2-10-488 | S | N—Ph | b1 | H | b1 | b5 |
| 2-10-489 | S | N—Ph | b2 | H | b2 | b5 |
| 2-10-490 | S | N—Ph | b3 | H | b3 | b5 |
| 2-10-491 | S | N—Ph | H | H | H | b6 |
| 2-10-492 | S | N—Ph | t-bu | H | t-bu | b6 |
| 2-10-493 | S | N—Ph | methyl | H | methyl | b6 |
| 2-10-494 | S | N—Ph | b1 | H | b1 | b6 |
| 2-10-495 | S | N—Ph | b2 | H | b2 | b6 |
| 2-10-496 | S | N—Ph | b3 | H | b3 | b6 |
| 2-10-497 | S | N—Ph | H | H | H | b7 |
| 2-10-498 | S | N—Ph | t-bu | H | t-bu | b7 |
| 2-10-499 | S | N—Ph | methyl | H | methyl | b7 |
| 2-10-500 | S | N—Ph | b1 | H | b1 | b7 |
| 2-10-501 | S | N—Ph | b2 | H | b2 | b7 |
| 2-10-502 | S | N—Ph | b3 | H | b3 | b7 |
| 2-10-503 | S | N—Ph | H | H | H | b8 |
| 2-10-504 | S | N—Ph | t-bu | H | t-bu | b8 |
| 2-10-505 | S | N—Ph | methyl | H | methyl | b8 |
| 2-10-506 | S | N—Ph | b1 | H | b1 | b8 |
| 2-10-507 | S | N—Ph | b2 | H | b2 | b8 |
| 2-10-508 | S | N—Ph | b3 | H | b3 | b8 |
| 2-10-509 | S | N—Ph | H | H | H | b9 |
| 2-10-510 | S | N—Ph | t-bu | H | t-bu | b9 |
| 2-10-511 | S | N—Ph | methyl | H | methyl | b9 |
| 2-10-512 | S | N—Ph | b1 | H | b1 | b9 |
| 2-10-513 | S | N—Ph | b2 | H | b2 | b9 |
| 2-10-514 | S | N—Ph | b3 | H | b3 | b9 |
| 2-10-515 | S | N—Ph | H | H | H | b10 |
| 2-10-516 | S | N—Ph | t-bu | H | t-bu | b10 |
| 2-10-517 | S | N—Ph | methyl | H | methyl | b10 |
| 2-10-518 | S | N—Ph | b1 | H | b1 | b10 |
| 2-10-519 | S | N—Ph | b2 | H | b2 | b10 |
| 2-10-520 | S | N—Ph | b3 | H | b3 | b10 |

### Group 2-11

The compounds are represented by the formula 2-11 below, wherein CY₂ is a benzene group, X₁ is B, and Y1, Y2, R1a, R1c, R3a, (R2)a2 are each shown in the table 11 below,

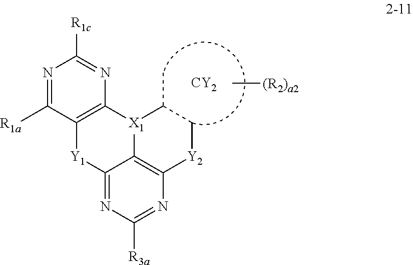

TABLE 11

| Compound No. | Y1 | Y2 | R1a | R1c | R3a | (R2)a2 |
|---|---|---|---|---|---|---|
| 2-11-001 | N—Ph | N—Ph | H | H | H | H |
| 2-11-002 | N—Ph | N—Ph | t-bu | H | t-bu | H |
| 2-11-003 | N—Ph | N—Ph | methyl | H | methyl | H |
| 2-11-004 | N—Ph | N—Ph | t-bu | t-bu | t-bu | t-bu |
| 2-11-005 | N—Ph | N—Ph | methyl | methyl | methyl | methyl |
| 2-11-006 | N—Ph | N—Ph | b1 | H | b1 | H |
| 2-11-007 | N—Ph | N—Ph | b2 | H | b2 | H |
| 2-11-008 | N—Ph | N—Ph | b3 | H | b3 | H |
| 2-11-009 | N—Ph | N—Ph | H | H | H | a1 |
| 2-11-010 | N—Ph | N—Ph | t-bu | t-bu | t-bu | a1 |
| 2-11-011 | N—Ph | N—Ph | methyl | methyl | methyl | a1 |
| 2-11-012 | N—Ph | N—Ph | b1 | b1 | b1 | a1 |
| 2-11-013 | N—Ph | N—Ph | b2 | b2 | b2 | a1 |
| 2-11-014 | N—Ph | N—Ph | b3 | b3 | b3 | a1 |
| 2-11-015 | N—Ph | N—Ph | H | H | H | a2 |
| 2-11-016 | N—Ph | N—Ph | t-bu | t-bu | t-bu | a2 |
| 2-11-017 | N—Ph | N—Ph | methyl | methyl | methyl | a2 |
| 2-11-018 | N—Ph | N—Ph | b1 | b1 | b1 | a2 |
| 2-11-019 | N—Ph | N—Ph | b2 | b2 | b2 | a2 |
| 2-11-020 | N—Ph | N—Ph | b3 | b3 | b3 | a2 |
| 2-11-021 | N—Ph | N—Ph | H | H | H | a3 |
| 2-11-022 | N—Ph | N—Ph | t-bu | t-bu | t-bu | a3 |
| 2-11-023 | N—Ph | N—Ph | methyl | methyl | methyl | a3 |
| 2-11-024 | N—Ph | N—Ph | b1 | b1 | b1 | a3 |
| 2-11-025 | N—Ph | N—Ph | b2 | b2 | b2 | a3 |
| 2-11-026 | N—Ph | N—Ph | b3 | b3 | b3 | a3 |
| 2-11-027 | N—Ph | N—Ph | H | H | H | a4 |
| 2-11-028 | N—Ph | N—Ph | t-bu | t-bu | t-bu | a4 |
| 2-11-029 | N—Ph | N—Ph | methyl | methyl | methyl | a4 |
| 2-11-030 | N—Ph | N—Ph | b1 | b1 | b1 | a4 |
| 2-11-031 | N—Ph | N—Ph | b2 | b2 | b2 | a4 |
| 2-11-032 | N—Ph | N—Ph | b3 | b3 | b3 | a4 |
| 2-11-033 | N—Ph | N—Ph | H | H | H | a5 |
| 2-11-034 | N—Ph | N—Ph | t-bu | t-bu | t-bu | a5 |
| 2-11-035 | N—Ph | N—Ph | methyl | methyl | methyl | a5 |
| 2-11-036 | N—Ph | N—Ph | b1 | b1 | b1 | a5 |
| 2-11-037 | N—Ph | N—Ph | b2 | b2 | b2 | a5 |
| 2-11-038 | N—Ph | N—Ph | b3 | b3 | b3 | a5 |
| 2-11-039 | N—Ph | N—Ph | H | H | H | a6 |
| 2-11-040 | N—Ph | N—Ph | t-bu | t-bu | t-bu | a6 |
| 2-11-041 | N—Ph | N—Ph | methyl | methyl | methyl | a6 |
| 2-11-042 | N—Ph | N—Ph | b1 | b1 | b1 | a6 |
| 2-11-043 | N—Ph | N—Ph | b2 | b2 | b2 | a6 |
| 2-11-044 | N—Ph | N—Ph | b3 | b3 | b3 | a6 |
| 2-11-045 | N—Ph | N—Ph | H | H | H | b1 |
| 2-11-046 | N—Ph | N—Ph | t-bu | t-bu | t-bu | b1 |
| 2-11-047 | N—Ph | N—Ph | methyl | methyl | methyl | b1 |
| 2-11-048 | N—Ph | N—Ph | b1 | b1 | b1 | b1 |
| 2-11-049 | N—Ph | N—Ph | b2 | b2 | b2 | b1 |
| 2-11-050 | N—Ph | N—Ph | b3 | b3 | b3 | b1 |
| 2-11-051 | N—Ph | N—Ph | H | H | H | b2 |
| 2-11-052 | N—Ph | N—Ph | t-bu | t-bu | t-bu | b2 |
| 2-11-053 | N—Ph | N—Ph | methyl | methyl | methyl | b2 |
| 2-11-054 | N—Ph | N—Ph | b1 | b1 | b1 | b2 |
| 2-11-055 | N—Ph | N—Ph | b2 | b2 | b2 | b2 |
| 2-11-056 | N—Ph | N—Ph | b3 | b3 | b3 | b2 |
| 2-11-057 | N—Ph | N—Ph | H | H | H | b3 |
| 2-11-058 | N—Ph | N—Ph | t-bu | t-bu | t-bu | b3 |
| 2-11-059 | N—Ph | N—Ph | methyl | methyl | methyl | b3 |
| 2-11-060 | N—Ph | N—Ph | b1 | b1 | b1 | b3 |
| 2-11-061 | N—Ph | N—Ph | b2 | b2 | b2 | b3 |
| 2-11-062 | N—Ph | N—Ph | b3 | b3 | b3 | b3 |
| 2-11-063 | N—Ph | N—Ph | H | H | H | b4 |
| 2-11-064 | N—Ph | N—Ph | t-bu | t-bu | t-bu | b4 |
| 2-11-065 | N—Ph | N—Ph | methyl | methyl | methyl | b4 |
| 2-11-066 | N—Ph | N—Ph | b1 | b1 | b1 | b4 |
| 2-11-067 | N—Ph | N—Ph | b2 | b2 | b2 | b4 |
| 2-11-068 | N—Ph | N—Ph | b3 | b3 | b3 | b4 |
| 2-11-069 | N—Ph | N—Ph | H | H | H | b5 |
| 2-11-070 | N—Ph | N—Ph | t-bu | t-bu | t-bu | b5 |
| 2-11-071 | N—Ph | N—Ph | methyl | methyl | methyl | b5 |
| 2-11-072 | N—Ph | N—Ph | b1 | b1 | b1 | b5 |
| 2-11-073 | N—Ph | N—Ph | b2 | b2 | b2 | b5 |
| 2-11-074 | N—Ph | N—Ph | b3 | b3 | b3 | b5 |
| 2-11-075 | N—Ph | N—Ph | H | H | H | b6 |
| 2-11-076 | N—Ph | N—Ph | t-bu | t-bu | t-bu | b6 |
| 2-11-077 | N—Ph | N—Ph | methyl | methyl | methyl | b6 |
| 2-11-078 | N—Ph | N—Ph | b1 | b1 | b1 | b6 |
| 2-11-079 | N—Ph | N—Ph | b2 | b2 | b2 | b6 |
| 2-11-080 | N—Ph | N—Ph | b3 | b3 | b3 | b6 |
| 2-11-081 | N—Ph | N—Ph | H | H | H | b7 |
| 2-11-082 | N—Ph | N—Ph | t-bu | t-bu | t-bu | b7 |
| 2-11-083 | N—Ph | N—Ph | methyl | methyl | methyl | b7 |
| 2-11-084 | N—Ph | N—Ph | b1 | b1 | b1 | b7 |
| 2-11-085 | N—Ph | N—Ph | b2 | b2 | b2 | b7 |
| 2-11-086 | N—Ph | N—Ph | b3 | b3 | b3 | b7 |
| 2-11-087 | N—Ph | N—Ph | H | H | H | b8 |
| 2-11-088 | N—Ph | N—Ph | t-bu | t-bu | t-bu | b8 |
| 2-11-089 | N—Ph | N—Ph | methyl | methyl | methyl | b8 |
| 2-11-090 | N—Ph | N—Ph | b1 | b1 | b1 | b8 |
| 2-11-091 | N—Ph | N—Ph | b2 | b2 | b2 | b8 |
| 2-11-092 | N—Ph | N—Ph | b3 | b3 | b3 | b8 |
| 2-11-093 | N—Ph | N—Ph | H | H | H | b9 |
| 2-11-094 | N—Ph | N—Ph | t-bu | t-bu | t-bu | b9 |
| 2-11-095 | N—Ph | N—Ph | methyl | methyl | methyl | b9 |
| 2-11-096 | N—Ph | N—Ph | b1 | b1 | b1 | b9 |
| 2-11-097 | N—Ph | N—Ph | b2 | b2 | b2 | b9 |
| 2-11-098 | N—Ph | N—Ph | b3 | b3 | b3 | b9 |
| 2-11-099 | N—Ph | N—Ph | H | H | H | b10 |
| 2-11-100 | N—Ph | N—Ph | t-bu | t-bu | t-bu | b10 |
| 2-11-101 | N—Ph | N—Ph | methyl | methyl | methyl | b10 |
| 2-11-102 | N—Ph | N—Ph | b1 | b1 | b1 | b10 |
| 2-11-103 | N—Ph | N—Ph | b2 | b2 | b2 | b10 |
| 2-11-104 | N—Ph | N—Ph | b3 | b3 | b3 | b10 |
| 2-11-105 | N-tolyl | N-tolyl | H | H | H | H |
| 2-11-106 | N-tolyl | N-tolyl | t-bu | H | t-bu | H |
| 2-11-107 | N-tolyl | N-tolyl | methyl | H | methyl | H |
| 2-11-108 | N-tolyl | N-tolyl | t-bu | t-bu | t-bu | t-bu |
| 2-11-109 | N-tolyl | N-tolyl | methyl | methyl | methyl | methyl |
| 2-11-110 | N-tolyl | N-tolyl | b1 | H | b1 | H |
| 2-11-111 | N-tolyl | N-tolyl | b2 | H | b2 | H |
| 2-11-112 | N-tolyl | N-tolyl | b3 | H | b3 | H |
| 2-11-113 | N-tolyl | N-tolyl | H | H | H | a1 |
| 2-11-114 | N-tolyl | N-tolyl | t-bu | t-bu | t-bu | a1 |
| 2-11-115 | N-tolyl | N-tolyl | methyl | methyl | methyl | a1 |
| 2-11-116 | N-tolyl | N-tolyl | b1 | b1 | b1 | a1 |
| 2-11-117 | N-tolyl | N-tolyl | b2 | b2 | b2 | a1 |
| 2-11-118 | N-tolyl | N-tolyl | b3 | b3 | b3 | a1 |
| 2-11-119 | N-tolyl | N-tolyl | H | H | H | a2 |
| 2-11-120 | N-tolyl | N-tolyl | t-bu | t-bu | t-bu | a2 |
| 2-11-121 | N-tolyl | N-tolyl | methyl | methyl | methyl | a2 |
| 2-11-122 | N-tolyl | N-tolyl | b1 | b1 | b1 | a2 |
| 2-11-123 | N-tolyl | N-tolyl | b2 | b2 | b2 | a2 |
| 2-11-124 | N-tolyl | N-tolyl | b3 | b3 | b3 | a2 |
| 2-11-125 | N-tolyl | N-tolyl | H | H | H | a3 |
| 2-11-126 | N-tolyl | N-tolyl | t-bu | t-bu | t-bu | a3 |
| 2-11-127 | N-tolyl | N-tolyl | methyl | methyl | methyl | a3 |
| 2-11-128 | N-tolyl | N-tolyl | b1 | b1 | b1 | a3 |
| 2-11-129 | N-tolyl | N-tolyl | b2 | b2 | b2 | a3 |
| 2-11-130 | N-tolyl | N-tolyl | b3 | b3 | b3 | a3 |
| 2-11-131 | N-tolyl | N-tolyl | H | H | H | a4 |
| 2-11-132 | N-tolyl | N-tolyl | t-bu | t-bu | t-bu | a4 |
| 2-11-133 | N-tolyl | N-tolyl | methyl | methyl | methyl | a4 |
| 2-11-134 | N-tolyl | N-tolyl | b1 | b1 | b1 | a4 |
| 2-11-135 | N-tolyl | N-tolyl | b2 | b2 | b2 | a4 |
| 2-11-136 | N-tolyl | N-tolyl | b3 | b3 | b3 | a4 |
| 2-11-137 | N-tolyl | N-tolyl | H | H | H | a5 |
| 2-11-138 | N-tolyl | N-tolyl | t-bu | t-bu | t-bu | a5 |
| 2-11-139 | N-tolyl | N-tolyl | methyl | methyl | methyl | a5 |
| 2-11-140 | N-tolyl | N-tolyl | b1 | b1 | b1 | a5 |
| 2-11-141 | N-tolyl | N-tolyl | b2 | b2 | b2 | a5 |
| 2-11-142 | N-tolyl | N-tolyl | b3 | b3 | b3 | a5 |
| 2-11-143 | N-tolyl | N-tolyl | H | H | H | a6 |
| 2-11-144 | N-tolyl | N-tolyl | t-bu | t-bu | t-bu | a6 |
| 2-11-145 | N-tolyl | N-tolyl | methyl | methyl | methyl | a6 |
| 2-11-146 | N-tolyl | N-tolyl | b1 | b1 | b1 | a6 |
| 2-11-147 | N-tolyl | N-tolyl | b2 | b2 | b2 | a6 |
| 2-11-148 | N-tolyl | N-tolyl | b3 | b3 | b3 | a6 |
| 2-11-149 | N-tolyl | N-tolyl | H | H | H | b1 |
| 2-11-150 | N-tolyl | N-tolyl | t-bu | t-bu | t-bu | b1 |
| 2-11-151 | N-tolyl | N-tolyl | methyl | methyl | methyl | b1 |
| 2-11-152 | N-tolyl | N-tolyl | b1 | b1 | b1 | b1 |
| 2-11-153 | N-tolyl | N-tolyl | b2 | b2 | b2 | b1 |
| 2-11-154 | N-tolyl | N-tolyl | b3 | b3 | b3 | b1 |
| 2-11-155 | N-tolyl | N-tolyl | H | H | H | b2 |
| 2-11-156 | N-tolyl | N-tolyl | t-bu | t-bu | t-bu | b2 |
| 2-11-157 | N-tolyl | N-tolyl | methyl | methyl | methyl | b2 |
| 2-11-158 | N-tolyl | N-tolyl | b1 | b1 | b1 | b2 |
| 2-11-159 | N-tolyl | N-tolyl | b2 | b2 | b2 | b2 |
| 2-11-160 | N-tolyl | N-tolyl | b3 | b3 | b3 | b2 |
| 2-11-161 | N-tolyl | N-tolyl | H | H | H | b3 |
| 2-11-162 | N-tolyl | N-tolyl | t-bu | t-bu | t-bu | b3 |
| 2-11-163 | N-tolyl | N-tolyl | methyl | methyl | methyl | b3 |
| 2-11-164 | N-tolyl | N-tolyl | b1 | b1 | b1 | b3 |
| 2-11-165 | N-tolyl | N-tolyl | b2 | b2 | b2 | b3 |
| 2-11-166 | N-tolyl | N-tolyl | b3 | b3 | b3 | b3 |
| 2-11-167 | N-tolyl | N-tolyl | H | H | H | b4 |
| 2-11-168 | N-tolyl | N-tolyl | t-bu | t-bu | t-bu | b4 |
| 2-11-169 | N-tolyl | N-tolyl | methyl | methyl | methyl | b4 |
| 2-11-170 | N-tolyl | N-tolyl | b1 | b1 | b1 | b4 |
| 2-11-171 | N-tolyl | N-tolyl | b2 | b2 | b2 | b4 |
| 2-11-172 | N-tolyl | N-tolyl | b3 | b3 | b3 | b4 |
| 2-11-173 | N-tolyl | N-tolyl | H | H | H | b5 |
| 2-11-174 | N-tolyl | N-tolyl | t-bu | t-bu | t-bu | b5 |
| 2-11-175 | N-tolyl | N-tolyl | methyl | methyl | methyl | b5 |
| 2-11-176 | N-tolyl | N-tolyl | b1 | b1 | b1 | b5 |
| 2-11-177 | N-tolyl | N-tolyl | b2 | b2 | b2 | b5 |
| 2-11-178 | N-tolyl | N-tolyl | b3 | b3 | b3 | b5 |
| 2-11-179 | N-tolyl | N-tolyl | H | H | H | b6 |
| 2-11-180 | N-tolyl | N-tolyl | t-bu | t-bu | t-bu | b6 |
| 2-11-181 | N-tolyl | N-tolyl | methyl | methyl | methyl | b6 |
| 2-11-182 | N-tolyl | N-tolyl | b1 | b1 | b1 | b6 |
| 2-11-183 | N-tolyl | N-tolyl | b2 | b2 | b2 | b6 |
| 2-11-184 | N-tolyl | N-tolyl | b3 | b3 | b3 | b6 |
| 2-11-185 | N-tolyl | N-tolyl | H | H | H | b7 |
| 2-11-186 | N-tolyl | N-tolyl | t-bu | t-bu | t-bu | b7 |
| 2-11-187 | N-tolyl | N-tolyl | methyl | methyl | methyl | b7 |
| 2-11-188 | N-tolyl | N-tolyl | b1 | b1 | b1 | b7 |
| 2-11-189 | N-tolyl | N-tolyl | b2 | b2 | b2 | b7 |
| 2-11-190 | N-tolyl | N-tolyl | b3 | b3 | b3 | b7 |
| 2-11-191 | N-tolyl | N-tolyl | H | H | H | b8 |
| 2-11-192 | N-tolyl | N-tolyl | t-bu | t-bu | t-bu | b8 |
| 2-11-193 | N-tolyl | N-tolyl | methyl | methyl | methyl | b8 |
| 2-11-194 | N-tolyl | N-tolyl | b1 | b1 | b1 | b8 |
| 2-11-195 | N-tolyl | N-tolyl | b2 | b2 | b2 | b8 |
| 2-11-196 | N-tolyl | N-tolyl | b3 | b3 | b3 | b8 |
| 2-11-197 | N-tolyl | N-tolyl | H | H | H | b9 |
| 2-11-198 | N-tolyl | N-tolyl | t-bu | t-bu | t-bu | b9 |
| 2-11-199 | N-tolyl | N-tolyl | methyl | methyl | methyl | b9 |
| 2-11-200 | N-tolyl | N-tolyl | b1 | b1 | b1 | b9 |
| 2-11-201 | N-tolyl | N-tolyl | b2 | b2 | b2 | b9 |
| 2-11-202 | N-tolyl | N-tolyl | b3 | b3 | b3 | b9 |
| 2-11-203 | N-tolyl | N-tolyl | H | H | H | b10 |
| 2-11-204 | N-tolyl | N-tolyl | t-bu | t-bu | t-bu | b10 |
| 2-11-205 | N-tolyl | N-tolyl | methyl | methyl | methyl | b10 |
| 2-11-206 | N-tolyl | N-tolyl | b1 | b1 | b1 | b10 |
| 2-11-207 | N-tolyl | N-tolyl | b2 | b2 | b2 | b10 |
| 2-11-208 | N-tolyl | N-tolyl | b3 | b3 | b3 | b10 |
| 2-11-209 | N-Biph | N-Biph | H | H | H | H |
| 2-11-210 | N-Biph | N-Biph | t-bu | H | t-bu | H |
| 2-11-211 | N-Biph | N-Biph | methyl | H | methyl | H |
| 2-11-212 | N-Biph | N-Biph | t-bu | t-bu | t-bu | t-bu |
| 2-11-213 | N-Biph | N-Biph | methyl | methyl | methyl | methyl |
| 2-11-214 | N-Biph | N-Biph | b1 | H | b1 | H |
| 2-11-215 | N-Biph | N-Biph | b2 | H | b2 | H |
| 2-11-216 | N-Biph | N-Biph | b3 | H | b3 | H |
| 2-11-217 | N-Biph | N-Biph | H | H | H | a1 |
| 2-11-218 | N-Biph | N-Biph | t-bu | t-bu | t-bu | a1 |
| 2-11-219 | N-Biph | N-Biph | methyl | methyl | methyl | a1 |
| 2-11-220 | N-Biph | N-Biph | b1 | b1 | b1 | a1 |
| 2-11-221 | N-Biph | N-Biph | b2 | b2 | b2 | a1 |
| 2-11-222 | N-Biph | N-Biph | b3 | b3 | b3 | a1 |
| 2-11-223 | N-Biph | N-Biph | H | H | H | a2 |
| 2-11-224 | N-Biph | N-Biph | t-bu | t-bu | t-bu | a2 |
| 2-11-225 | N-Biph | N-Biph | methyl | methyl | methyl | a2 |
| 2-11-226 | N-Biph | N-Biph | b1 | b1 | b1 | a2 |
| 2-11-227 | N-Biph | N-Biph | b2 | b2 | b2 | a2 |
| 2-11-228 | N-Biph | N-Biph | b3 | b3 | b3 | a2 |
| 2-11-229 | N-Biph | N-Biph | H | H | H | a3 |
| 2-11-230 | N-Biph | N-Biph | t-bu | t-bu | t-bu | a3 |
| 2-11-231 | N-Biph | N-Biph | methyl | methyl | methyl | a3 |
| 2-11-232 | N-Biph | N-Biph | b1 | b1 | b1 | a3 |
| 2-11-233 | N-Biph | N-Biph | b2 | b2 | b2 | a3 |
| 2-11-234 | N-Biph | N-Biph | b3 | b3 | b3 | a3 |
| 2-11-235 | N-Biph | N-Biph | H | H | H | a4 |
| 2-11-236 | N-Biph | N-Biph | t-bu | t-bu | t-bu | a4 |
| 2-11-237 | N-Biph | N-Biph | methyl | methyl | methyl | a4 |
| 2-11-238 | N-Biph | N-Biph | b1 | b1 | b1 | a4 |
| 2-11-239 | N-Biph | N-Biph | b2 | b2 | b2 | a4 |
| 2-11-240 | N-Biph | N-Biph | b3 | b3 | b3 | a4 |
| 2-11-241 | N-Biph | N-Biph | H | H | H | a5 |
| 2-11-242 | N-Biph | N-Biph | t-bu | t-bu | t-bu | a5 |
| 2-11-243 | N-Biph | N-Biph | methyl | methyl | methyl | a5 |
| 2-11-244 | N-Biph | N-Biph | b1 | b1 | b1 | a5 |
| 2-11-245 | N-Biph | N-Biph | b2 | b2 | b2 | a5 |
| 2-11-246 | N-Biph | N-Biph | b3 | b3 | b3 | a5 |
| 2-11-247 | N-Biph | N-Biph | H | H | H | a6 |
| 2-11-248 | N-Biph | N-Biph | t-bu | t-bu | t-bu | a6 |
| 2-11-249 | N-Biph | N-Biph | methyl | methyl | methyl | a6 |
| 2-11-250 | N-Biph | N-Biph | b1 | b1 | b1 | a6 |
| 2-11-251 | N-Biph | N-Biph | b2 | b2 | b2 | a6 |
| 2-11-252 | N-Biph | N-Biph | b3 | b3 | b3 | a6 |
| 2-11-253 | N-Biph | N-Biph | H | H | H | b1 |
| 2-11-254 | N-Biph | N-Biph | t-bu | t-bu | t-bu | b1 |
| 2-11-255 | N-Biph | N-Biph | methyl | methyl | methyl | b1 |
| 2-11-256 | N-Biph | N-Biph | b1 | b1 | b1 | b1 |
| 2-11-257 | N-Biph | N-Biph | b2 | b2 | b2 | b1 |
| 2-11-258 | N-Biph | N-Biph | b3 | b3 | b3 | b1 |
| 2-11-259 | N-Biph | N-Biph | H | H | H | b2 |
| 2-11-260 | N-Biph | N-Biph | t-bu | t-bu | t-bu | b2 |
| 2-11-261 | N-Biph | N-Biph | methyl | methyl | methyl | b2 |
| 2-11-262 | N-Biph | N-Biph | b1 | b1 | b1 | b2 |
| 2-11-263 | N-Biph | N-Biph | b2 | b2 | b2 | b2 |
| 2-11-264 | N-Biph | N-Biph | b3 | b3 | b3 | b2 |
| 2-11-265 | N-Biph | N-Biph | H | H | H | b3 |
| 2-11-266 | N-Biph | N-Biph | t-bu | t-bu | t-bu | b3 |
| 2-11-267 | N-Biph | N-Biph | methyl | methyl | methyl | b3 |
| 2-11-268 | N-Biph | N-Biph | b1 | b1 | b1 | b3 |
| 2-11-269 | N-Biph | N-Biph | b2 | b2 | b2 | b3 |
| 2-11-270 | N-Biph | N-Biph | b3 | b3 | b3 | b3 |
| 2-11-271 | N-Biph | N-Biph | H | H | H | b4 |
| 2-11-272 | N-Biph | N-Biph | t-bu | t-bu | t-bu | b4 |
| 2-11-273 | N-Biph | N-Biph | methyl | methyl | methyl | b4 |
| 2-11-274 | N-Biph | N-Biph | b1 | b1 | b1 | b4 |
| 2-11-275 | N-Biph | N-Biph | b2 | b2 | b2 | b4 |
| 2-11-276 | N-Biph | N-Biph | b3 | b3 | b3 | b4 |
| 2-11-277 | N-Biph | N-Biph | H | H | H | b5 |
| 2-11-278 | N-Biph | N-Biph | t-bu | t-bu | t-bu | b5 |
| 2-11-279 | N-Biph | N-Biph | methyl | methyl | methyl | b5 |
| 2-11-280 | N-Biph | N-Biph | b1 | b1 | b1 | b5 |
| 2-11-281 | N-Biph | N-Biph | b2 | b2 | b2 | b5 |
| 2-11-282 | N-Biph | N-Biph | b3 | b3 | b3 | b5 |
| 2-11-283 | N-Biph | N-Biph | H | H | H | b6 |
| 2-11-284 | N-Biph | N-Biph | t-bu | t-bu | t-bu | b6 |
| 2-11-285 | N-Biph | N-Biph | methyl | methyl | methyl | b6 |
| 2-11-286 | N-Biph | N-Biph | b1 | b1 | b1 | b6 |
| 2-11-287 | N-Biph | N-Biph | b2 | b2 | b2 | b6 |
| 2-11-288 | N-Biph | N-Biph | b3 | b3 | b3 | b6 |
| 2-11-289 | N-Biph | N-Biph | H | H | H | b7 |
| 2-11-290 | N-Biph | N-Biph | t-bu | t-bu | t-bu | b7 |
| 2-11-291 | N-Biph | N-Biph | methyl | methyl | methyl | b7 |
| 2-11-292 | N-Biph | N-Biph | b1 | b1 | b1 | b7 |
| 2-11-293 | N-Biph | N-Biph | b2 | b2 | b2 | b7 |
| 2-11-294 | N-Biph | N-Biph | b3 | b3 | b3 | b7 |
| 2-11-295 | N-Biph | N-Biph | H | H | H | b8 |
| 2-11-296 | N-Biph | N-Biph | t-bu | t-bu | t-bu | b8 |
| 2-11-297 | N-Biph | N-Biph | methyl | methyl | methyl | b8 |
| 2-11-298 | N-Biph | N-Biph | b1 | b1 | b1 | b8 |
| 2-11-299 | N-Biph | N-Biph | b2 | b2 | b2 | b8 |
| 2-11-300 | N-Biph | N-Biph | b3 | b3 | b3 | b8 |
| 2-11-301 | N-Biph | N-Biph | H | H | H | b9 |
| 2-11-302 | N-Biph | N-Biph | t-bu | t-bu | t-bu | b9 |
| 2-11-303 | N-Biph | N-Biph | methyl | methyl | methyl | b9 |
| 2-11-304 | N-Biph | N-Biph | b1 | b1 | b1 | b9 |
| 2-11-305 | N-Biph | N-Biph | b2 | b2 | b2 | b9 |
| 2-11-306 | N-Biph | N-Biph | b3 | b3 | b3 | b9 |
| 2-11-307 | N-Biph | N-Biph | H | H | H | b10 |
| 2-11-308 | N-Biph | N-Biph | t-bu | t-bu | t-bu | b10 |
| 2-11-309 | N-Biph | N-Biph | methyl | methyl | methyl | b10 |
| 2-11-310 | N-Biph | N-Biph | b1 | b1 | b1 | b10 |
| 2-11-311 | N-Biph | N-Biph | b2 | b2 | b2 | b10 |
| 2-11-312 | N-Biph | N-Biph | b3 | b3 | b3 | b10 |
| 2-11-313 | O | N—Ph | H | H | H | H |
| 2-11-314 | O | N—Ph | t-bu | H | t-bu | H |
| 2-11-315 | O | N—Ph | methyl | H | methyl | H |
| 2-11-316 | O | N—Ph | t-bu | t-bu | t-bu | t-bu |
| 2-11-317 | O | N—Ph | methyl | methyl | methyl | methyl |
| 2-11-318 | O | N—Ph | b1 | H | b1 | H |
| 2-11-319 | O | N—Ph | b2 | H | b2 | H |
| 2-11-320 | O | N—Ph | b3 | H | b3 | H |
| 2-11-321 | O | N—Ph | H | H | H | a1 |
| 2-11-322 | O | N—Ph | t-bu | t-bu | t-bu | a1 |
| 2-11-323 | O | N—Ph | methyl | methyl | methyl | a1 |
| 2-11-324 | O | N—Ph | b1 | b1 | b1 | a1 |
| 2-11-325 | O | N—Ph | b2 | b2 | b2 | a1 |
| 2-11-326 | O | N—Ph | b3 | b3 | b3 | a1 |
| 2-11-327 | O | N—Ph | H | H | H | a2 |
| 2-11-328 | O | N—Ph | t-bu | t-bu | t-bu | a2 |
| 2-11-329 | O | N—Ph | methyl | methyl | methyl | a2 |
| 2-11-330 | O | N—Ph | b1 | b1 | b1 | a2 |
| 2-11-331 | O | N—Ph | b2 | b2 | b2 | a2 |
| 2-11-332 | O | N—Ph | b3 | b3 | b3 | a2 |
| 2-11-333 | O | N—Ph | H | H | H | a3 |
| 2-11-334 | O | N—Ph | t-bu | t-bu | t-bu | a3 |
| 2-11-335 | O | N—Ph | methyl | methyl | methyl | a3 |
| 2-11-336 | O | N—Ph | b1 | b1 | b1 | a3 |
| 2-11-337 | O | N—Ph | b2 | b2 | b2 | a3 |
| 2-11-338 | O | N—Ph | b3 | b3 | b3 | a3 |
| 2-11-339 | O | N—Ph | H | H | H | a4 |
| 2-11-340 | O | N—Ph | t-bu | t-bu | t-bu | a4 |
| 2-11-341 | O | N—Ph | methyl | methyl | methyl | a4 |
| 2-11-342 | O | N—Ph | b1 | b1 | b1 | a4 |
| 2-11-343 | O | N—Ph | b2 | b2 | b2 | a4 |
| 2-11-344 | O | N—Ph | b3 | b3 | b3 | a4 |
| 2-11-345 | O | N—Ph | H | H | H | a5 |
| 2-11-346 | O | N—Ph | t-bu | t-bu | t-bu | a5 |
| 2-11-347 | O | N—Ph | methyl | methyl | methyl | a5 |
| 2-11-348 | O | N—Ph | b1 | b1 | b1 | a5 |
| 2-11-349 | O | N—Ph | b2 | b2 | b2 | a5 |
| 2-11-350 | O | N—Ph | b3 | b3 | b3 | a5 |
| 2-11-351 | O | N—Ph | H | H | H | a6 |
| 2-11-352 | O | N—Ph | t-bu | t-bu | t-bu | a6 |
| 2-11-353 | O | N—Ph | methyl | methyl | methyl | a6 |
| 2-11-354 | O | N—Ph | b1 | b1 | b1 | a6 |
| 2-11-355 | O | N—Ph | b2 | b2 | b2 | a6 |
| 2-11-356 | O | N—Ph | b3 | b3 | b3 | a6 |
| 2-11-357 | O | N—Ph | H | H | H | b1 |
| 2-11-358 | O | N—Ph | t-bu | t-bu | t-bu | b1 |
| 2-11-359 | O | N—Ph | methyl | methyl | methyl | b1 |
| 2-11-360 | O | N—Ph | b1 | b1 | b1 | b1 |
| 2-11-361 | O | N—Ph | b2 | b2 | b2 | b1 |
| 2-11-362 | O | N—Ph | b3 | b3 | b3 | b1 |
| 2-11-363 | O | N—Ph | H | H | H | b2 |
| 2-11-364 | O | N—Ph | t-bu | t-bu | t-bu | b2 |
| 2-11-365 | O | N—Ph | methyl | methyl | methyl | b2 |
| 2-11-366 | O | N—Ph | b1 | b1 | b1 | b2 |
| 2-11-367 | O | N—Ph | b2 | b2 | b2 | b2 |
| 2-11-368 | O | N—Ph | b3 | b3 | b3 | b2 |
| 2-11-369 | O | N—Ph | H | H | H | b3 |
| 2-11-370 | O | N—Ph | t-bu | t-bu | t-bu | b3 |
| 2-11-371 | O | N—Ph | methyl | methyl | methyl | b3 |
| 2-11-372 | O | N—Ph | b1 | b1 | b1 | b3 |
| 2-11-373 | O | N—Ph | b2 | b2 | b2 | b3 |
| 2-11-374 | O | N—Ph | b3 | b3 | b3 | b3 |
| 2-11-375 | O | N—Ph | H | H | H | b4 |
| 2-11-376 | O | N—Ph | t-bu | t-bu | t-bu | b4 |
| 2-11-377 | O | N—Ph | methyl | methyl | methyl | b4 |
| 2-11-378 | O | N—Ph | b1 | b1 | b1 | b4 |
| 2-11-379 | O | N—Ph | b2 | b2 | b2 | b4 |
| 2-11-380 | O | N—Ph | b3 | b3 | b3 | b4 |
| 2-11-381 | O | N—Ph | H | H | H | b5 |
| 2-11-382 | O | N—Ph | t-bu | t-bu | t-bu | b5 |
| 2-11-383 | O | N—Ph | methyl | methyl | methyl | b5 |
| 2-11-384 | O | N—Ph | b1 | b1 | b1 | b5 |
| 2-11-385 | O | N—Ph | b2 | b2 | b2 | b5 |
| 2-11-386 | O | N—Ph | b3 | b3 | b3 | b5 |
| 2-11-387 | O | N—Ph | H | H | H | b6 |
| 2-11-388 | O | N—Ph | t-bu | t-bu | t-bu | b6 |
| 2-11-389 | O | N—Ph | methyl | methyl | methyl | b6 |
| 2-11-390 | O | N—Ph | b1 | b1 | b1 | b6 |
| 2-11-391 | O | N—Ph | b2 | b2 | b2 | b6 |
| 2-11-392 | O | N—Ph | b3 | b3 | b3 | b6 |
| 2-11-393 | O | N—Ph | H | H | H | b7 |
| 2-11-394 | O | N—Ph | t-bu | t-bu | t-bu | b7 |
| 2-11-395 | O | N—Ph | methyl | methyl | methyl | b7 |
| 2-11-396 | O | N—Ph | b1 | b1 | b1 | b7 |
| 2-11-397 | O | N—Ph | b2 | b2 | b2 | b7 |
| 2-11-398 | O | N—Ph | b3 | b3 | b3 | b7 |
| 2-11-399 | O | N—Ph | H | H | H | b8 |
| 2-11-400 | O | N—Ph | t-bu | t-bu | t-bu | b8 |
| 2-11-401 | O | N—Ph | methyl | methyl | methyl | b8 |
| 2-11-402 | O | N—Ph | b1 | b1 | b1 | b8 |
| 2-11-403 | O | N—Ph | b2 | b2 | b2 | b8 |
| 2-11-404 | O | N—Ph | b3 | b3 | b3 | b8 |
| 2-11-405 | O | N—Ph | H | H | H | b9 |
| 2-11-406 | O | N—Ph | t-bu | t-bu | t-bu | b9 |
| 2-11-407 | O | N—Ph | methyl | methyl | methyl | b9 |
| 2-11-408 | O | N—Ph | b1 | b1 | b1 | b9 |
| 2-11-409 | O | N—Ph | b2 | b2 | b2 | b9 |
| 2-11-410 | O | N—Ph | b3 | b3 | b3 | b9 |
| 2-11-411 | O | N—Ph | H | H | H | b10 |
| 2-11-412 | O | N—Ph | t-bu | t-bu | t-bu | b10 |
| 2-11-413 | O | N—Ph | methyl | methyl | methyl | b10 |
| 2-11-414 | O | N—Ph | b1 | b1 | b1 | b10 |
| 2-11-415 | O | N—Ph | b2 | b2 | b2 | b10 |
| 2-11-416 | O | N—Ph | b3 | b3 | b3 | b10 |
| 2-11-417 | S | N—Ph | H | H | H | H |
| 2-11-418 | S | N—Ph | t-bu | H | t-bu | H |
| 2-11-419 | S | N—Ph | methyl | H | methyl | H |
| 2-11-420 | S | N—Ph | t-bu | t-bu | t-bu | t-bu |
| 2-11-421 | S | N—Ph | methyl | methyl | methyl | methyl |
| 2-11-422 | S | N—Ph | b1 | H | b1 | H |
| 2-11-423 | S | N—Ph | b2 | H | b2 | H |
| 2-11-424 | S | N—Ph | b3 | H | b3 | H |
| 2-11-425 | S | N—Ph | H | H | H | a1 |
| 2-11-426 | S | N—Ph | t-bu | t-bu | t-bu | a1 |
| 2-11-427 | S | N—Ph | methyl | methyl | methyl | a1 |
| 2-11-428 | S | N—Ph | b1 | b1 | b1 | a1 |
| 2-11-429 | S | N—Ph | b2 | b2 | b2 | a1 |
| 2-11-430 | S | N—Ph | b3 | b3 | b3 | a1 |
| 2-11-431 | S | N—Ph | H | H | H | a2 |
| 2-11-432 | S | N—Ph | t-bu | t-bu | t-bu | a2 |
| 2-11-433 | S | N—Ph | methyl | methyl | methyl | a2 |
| 2-11-434 | S | N—Ph | b1 | b1 | b1 | a2 |
| 2-11-435 | S | N—Ph | b2 | b2 | b2 | a2 |
| 2-11-436 | S | N—Ph | b3 | b3 | b3 | a2 |
| 2-11-437 | S | N—Ph | H | H | H | a3 |
| 2-11-438 | S | N—Ph | t-bu | t-bu | t-bu | a3 |
| 2-11-439 | S | N—Ph | methyl | methyl | methyl | a3 |
| 2-11-440 | S | N—Ph | b1 | b1 | b1 | a3 |
| 2-11-441 | S | N—Ph | b2 | b2 | b2 | a3 |
| 2-11-442 | S | N—Ph | b3 | b3 | b3 | a3 |
| 2-11-443 | S | N—Ph | H | H | H | a4 |
| 2-11-444 | S | N—Ph | t-bu | t-bu | t-bu | a4 |
| 2-11-445 | S | N—Ph | methyl | methyl | methyl | a4 |
| 2-11-446 | S | N—Ph | b1 | b1 | b1 | a4 |
| 2-11-447 | S | N—Ph | b2 | b2 | b2 | a4 |
| 2-11-448 | S | N—Ph | b3 | b3 | b3 | a4 |
| 2-11-449 | S | N—Ph | H | H | H | a5 |
| 2-11-450 | S | N—Ph | t-bu | t-bu | t-bu | a5 |
| 2-11-451 | S | N—Ph | methyl | methyl | methyl | a5 |
| 2-11-452 | S | N—Ph | b1 | b1 | b1 | a5 |
| 2-11-453 | S | N—Ph | b2 | b2 | b2 | a5 |
| 2-11-454 | S | N—Ph | b3 | b3 | b3 | a5 |
| 2-11-455 | S | N—Ph | H | H | H | a6 |
| 2-11-456 | S | N—Ph | t-bu | t-bu | t-bu | a6 |
| 2-11-457 | S | N—Ph | methyl | methyl | methyl | a6 |
| 2-11-458 | S | N—Ph | b1 | b1 | b1 | a6 |
| 2-11-459 | S | N—Ph | b2 | b2 | b2 | a6 |
| 2-11-460 | S | N—Ph | b3 | b3 | b3 | a6 |
| 2-11-461 | S | N—Ph | H | H | H | b1 |
| 2-11-462 | S | N—Ph | t-bu | t-bu | t-bu | b1 |
| 2-11-463 | S | N—Ph | methyl | methyl | methyl | b1 |
| 2-11-464 | S | N—Ph | b1 | b1 | b1 | b1 |
| 2-11-465 | S | N—Ph | b2 | b2 | b2 | b1 |
| 2-11-466 | S | N—Ph | b3 | b3 | b3 | b1 |
| 2-11-467 | S | N—Ph | H | H | H | b2 |
| 2-11-468 | S | N—Ph | t-bu | t-bu | t-bu | b2 |
| 2-11-469 | S | N—Ph | methyl | methyl | methyl | b2 |
| 2-11-470 | S | N—Ph | b1 | b1 | b1 | b2 |
| 2-11-471 | S | N—Ph | b2 | b2 | b2 | b2 |
| 2-11-472 | S | N—Ph | b3 | b3 | b3 | b2 |
| 2-11-473 | S | N—Ph | H | H | H | b3 |
| 2-11-474 | S | N—Ph | t-bu | t-bu | t-bu | b3 |
| 2-11-475 | S | N—Ph | methyl | methyl | methyl | b3 |
| 2-11-476 | S | N—Ph | b1 | b1 | b1 | b3 |
| 2-11-477 | S | N—Ph | b2 | b2 | b2 | b3 |
| 2-11-478 | S | N—Ph | b3 | b3 | b3 | b3 |
| 2-11-479 | S | N—Ph | H | H | H | b4 |
| 2-11-480 | S | N—Ph | t-bu | t-bu | t-bu | b4 |
| 2-11-481 | S | N—Ph | methyl | methyl | methyl | b4 |
| 2-11-482 | S | N—Ph | b1 | b1 | b1 | b4 |
| 2-11-483 | S | N—Ph | b2 | b2 | b2 | b4 |
| 2-11-484 | S | N—Ph | b3 | b3 | b3 | b4 |
| 2-11-485 | S | N—Ph | H | H | H | b5 |
| 2-11-486 | S | N—Ph | t-bu | t-bu | t-bu | b5 |
| 2-11-487 | S | N—Ph | methyl | methyl | methyl | b5 |
| 2-11-488 | S | N—Ph | b1 | b1 | b1 | b5 |
| 2-11-489 | S | N—Ph | b2 | b2 | b2 | b5 |
| 2-11-490 | S | N—Ph | b3 | b3 | b3 | b5 |
| 2-11-491 | S | N—Ph | H | H | H | b6 |
| 2-11-492 | S | N—Ph | t-bu | t-bu | t-bu | b6 |
| 2-11-493 | S | N—Ph | methyl | methyl | methyl | b6 |
| 2-11-494 | S | N—Ph | b1 | b1 | b1 | b6 |
| 2-11-495 | S | N—Ph | b2 | b2 | b2 | b6 |
| 2-11-496 | S | N—Ph | b3 | b3 | b3 | b6 |
| 2-11-497 | S | N—Ph | H | H | H | b7 |
| 2-11-498 | S | N—Ph | t-bu | t-bu | t-bu | b7 |
| 2-11-499 | S | N—Ph | methyl | methyl | methyl | b7 |
| 2-11-500 | S | N—Ph | b1 | b1 | b1 | b7 |
| 2-11-501 | S | N—Ph | b2 | b2 | b2 | b7 |
| 2-11-502 | S | N—Ph | b3 | b3 | b3 | b7 |
| 2-11-503 | S | N—Ph | H | H | H | b8 |
| 2-11-504 | S | N—Ph | t-bu | t-bu | t-bu | b8 |
| 2-11-505 | S | N—Ph | methyl | methyl | methyl | b8 |
| 2-11-506 | S | N—Ph | b1 | b1 | b1 | b8 |
| 2-11-507 | S | N—Ph | b2 | b2 | b2 | b8 |
| 2-11-508 | S | N—Ph | b3 | b3 | b3 | b8 |
| 2-11-509 | S | N—Ph | H | H | H | b9 |
| 2-11-510 | S | N—Ph | t-bu | t-bu | t-bu | b9 |
| 2-11-511 | S | N—Ph | methyl | methyl | methyl | b9 |
| 2-11-512 | S | N—Ph | b1 | b1 | b1 | b9 |
| 2-11-513 | S | N—Ph | b2 | b2 | b2 | b9 |
| 2-11-514 | S | N—Ph | b3 | b3 | b3 | b9 |
| 2-11-515 | S | N—Ph | H | H | H | b10 |
| 2-11-516 | S | N—Ph | t-bu | t-bu | t-bu | b10 |
| 2-11-517 | S | N—Ph | methyl | methyl | methyl | b10 |
| 2-11-518 | S | N—Ph | b1 | b1 | b1 | b10 |
| 2-11-519 | S | N—Ph | b2 | b2 | b2 | b10 |
| 2-11-520 | S | N—Ph | b3 | b3 | b3 | b10 |

### Group 2-12

The compounds are represented by the formula 2-12 below, wherein CY₁ is a benzene group, X₁ is B, and Y1, Y2, R2b, R2d, R3a, (R1)a1 are each shown in the table 12 below.

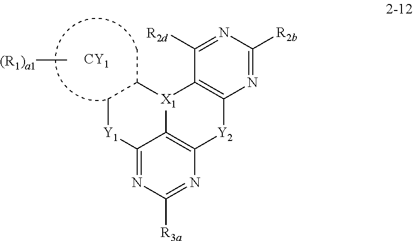

TABLE 12

| Compound No. | Y1 | Y2 | R2b | R2d | R3a | (R1)a1 |
|---|---|---|---|---|---|---|
| 2-12-001 | N—Ph | N—Ph | H | H | H | H |
| 2-12-002 | N—Ph | N—Ph | t-bu | H | t-bu | H |
| 2-12-003 | N—Ph | N—Ph | methyl | H | methyl | H |
| 2-12-004 | N—Ph | N—Ph | t-bu | t-bu | t-bu | t-bu |
| 2-12-005 | N—Ph | N—Ph | methyl | methyl | methyl | methyl |
| 2-12-006 | N—Ph | N—Ph | b1 | H | b1 | H |
| 2-12-007 | N—Ph | N—Ph | b2 | H | b2 | H |
| 2-12-008 | N—Ph | N—Ph | b3 | H | b3 | H |
| 2-12-009 | N—Ph | N—Ph | H | H | H | a1 |
| 2-12-010 | N—Ph | N—Ph | t-bu | H | t-bu | a1 |
| 2-12-011 | N—Ph | N—Ph | methyl | H | methyl | a1 |
| 2-12-012 | N—Ph | N—Ph | b1 | H | b1 | a1 |
| 2-12-013 | N—Ph | N—Ph | b2 | H | b2 | a1 |
| 2-12-014 | N—Ph | N—Ph | b3 | H | b3 | a1 |
| 2-12-015 | N—Ph | N—Ph | H | H | H | a2 |
| 2-12-016 | N—Ph | N—Ph | t-bu | H | t-bu | a2 |
| 2-12-017 | N—Ph | N—Ph | methyl | H | methyl | a2 |
| 2-12-018 | N—Ph | N—Ph | b1 | H | b1 | a2 |
| 2-12-019 | N—Ph | N—Ph | b2 | H | b2 | a2 |
| 2-12-020 | N—Ph | N—Ph | b3 | H | b3 | a2 |
| 2-12-021 | N—Ph | N—Ph | H | H | H | a3 |
| 2-12-022 | N—Ph | N—Ph | t-bu | H | t-bu | a3 |
| 2-12-023 | N—Ph | N—Ph | methyl | H | methyl | a3 |
| 2-12-024 | N—Ph | N—Ph | b1 | H | b1 | a3 |
| 2-12-025 | N—Ph | N—Ph | b2 | H | b2 | a3 |
| 2-12-026 | N—Ph | N—Ph | b3 | H | b3 | a3 |
| 2-12-027 | N—Ph | N—Ph | H | H | H | a4 |
| 2-12-028 | N—Ph | N—Ph | t-bu | H | t-bu | a4 |
| 2-12-029 | N—Ph | N—Ph | methyl | H | methyl | a4 |
| 2-12-030 | N—Ph | N—Ph | b1 | H | b1 | a4 |
| 2-12-031 | N—Ph | N—Ph | b2 | H | b2 | a4 |
| 2-12-032 | N—Ph | N—Ph | b3 | H | b3 | a4 |
| 2-12-033 | N—Ph | N—Ph | H | H | H | a5 |
| 2-12-034 | N—Ph | N—Ph | t-bu | H | t-bu | a5 |
| 2-12-035 | N—Ph | N—Ph | methyl | H | methyl | a5 |
| 2-12-036 | N—Ph | N—Ph | b1 | H | b1 | a5 |
| 2-12-037 | N—Ph | N—Ph | b2 | H | b2 | a5 |
| 2-12-038 | N—Ph | N—Ph | b3 | H | b3 | a5 |
| 2-12-039 | N—Ph | N—Ph | H | H | H | a6 |
| 2-12-040 | N—Ph | N—Ph | t-bu | H | t-bu | a6 |
| 2-12-041 | N—Ph | N—Ph | methyl | H | methyl | a6 |
| 2-12-042 | N—Ph | N—Ph | b1 | H | b1 | a6 |
| 2-12-043 | N—Ph | N—Ph | b2 | H | b2 | a6 |
| 2-12-044 | N—Ph | N—Ph | b3 | H | b3 | a6 |
| 2-12-045 | N—Ph | N—Ph | H | H | H | b1 |
| 2-12-046 | N—Ph | N—Ph | t-bu | H | t-bu | b1 |
| 2-12-047 | N—Ph | N—Ph | methyl | H | methyl | b1 |
| 2-12-048 | N—Ph | N—Ph | b1 | H | b1 | b1 |
| 2-12-049 | N—Ph | N—Ph | b2 | H | b2 | b1 |
| 2-12-050 | N—Ph | N—Ph | b3 | H | b3 | b1 |
| 2-12-051 | N—Ph | N—Ph | H | H | H | b2 |
| 2-12-052 | N—Ph | N—Ph | t-bu | H | t-bu | b2 |
| 2-12-053 | N—Ph | N—Ph | methyl | H | methyl | b2 |
| 2-12-054 | N—Ph | N—Ph | b1 | H | b1 | b2 |
| 2-12-055 | N—Ph | N—Ph | b2 | H | b2 | b2 |
| 2-12-056 | N—Ph | N—Ph | b3 | H | b3 | b2 |
| 2-12-057 | N—Ph | N—Ph | H | H | H | b3 |
| 2-12-058 | N—Ph | N—Ph | t-bu | H | t-bu | b3 |
| 2-12-059 | N—Ph | N—Ph | methyl | H | methyl | b3 |
| 2-12-060 | N—Ph | N—Ph | b1 | H | b1 | b3 |
| 2-12-061 | N—Ph | N—Ph | b2 | H | b2 | b3 |
| 2-12-062 | N—Ph | N—Ph | b3 | H | b3 | b3 |
| 2-12-063 | N—Ph | N—Ph | H | H | H | b4 |
| 2-12-064 | N—Ph | N—Ph | t-bu | H | t-bu | b4 |
| 2-12-065 | N—Ph | N—Ph | methyl | H | methyl | b4 |
| 2-12-066 | N—Ph | N—Ph | b1 | H | b1 | b4 |
| 2-12-067 | N—Ph | N—Ph | b2 | H | b2 | b4 |
| 2-12-068 | N—Ph | N—Ph | b3 | H | b3 | b4 |
| 2-12-069 | N—Ph | N—Ph | H | H | H | b5 |
| 2-12-070 | N—Ph | N—Ph | t-bu | H | t-bu | b5 |
| 2-12-071 | N—Ph | N—Ph | methyl | H | methyl | b5 |
| 2-12-072 | N—Ph | N—Ph | b1 | H | b1 | b5 |
| 2-12-073 | N—Ph | N—Ph | b2 | H | b2 | b5 |
| 2-12-074 | N—Ph | N—Ph | b3 | H | b3 | b5 |
| 2-12-075 | N—Ph | N—Ph | H | H | H | b6 |
| 2-12-076 | N—Ph | N—Ph | t-bu | H | t-bu | b6 |
| 2-12-077 | N—Ph | N—Ph | methyl | H | methyl | b6 |
| 2-12-078 | N—Ph | N—Ph | b1 | H | b1 | b6 |
| 2-12-079 | N—Ph | N—Ph | b2 | H | b2 | b6 |
| 2-12-080 | N—Ph | N—Ph | b3 | H | b3 | b6 |
| 2-12-081 | N—Ph | N—Ph | H | H | H | b7 |
| 2-12-082 | N—Ph | N—Ph | t-bu | H | t-bu | b7 |
| 2-12-083 | N—Ph | N—Ph | methyl | H | methyl | b7 |
| 2-12-084 | N—Ph | N—Ph | b1 | H | b1 | b7 |
| 2-12-085 | N—Ph | N—Ph | b2 | H | b2 | b7 |
| 2-12-086 | N—Ph | N—Ph | b3 | H | b3 | b7 |
| 2-12-087 | N—Ph | N—Ph | H | H | H | b8 |
| 2-12-088 | N—Ph | N—Ph | t-bu | H | t-bu | b8 |
| 2-12-089 | N—Ph | N—Ph | methyl | H | methyl | b8 |
| 2-12-090 | N—Ph | N—Ph | b1 | H | b1 | b8 |
| 2-12-091 | N—Ph | N—Ph | b2 | H | b2 | b8 |
| 2-12-092 | N—Ph | N—Ph | b3 | H | b3 | b8 |
| 2-12-093 | N—Ph | N—Ph | H | H | H | b9 |
| 2-12-094 | N—Ph | N—Ph | t-bu | H | t-bu | b9 |
| 2-12-095 | N—Ph | N—Ph | methyl | H | methyl | b9 |
| 2-12-096 | N—Ph | N—Ph | b1 | H | b1 | b9 |
| 2-12-097 | N—Ph | N—Ph | b2 | H | b2 | b9 |
| 2-12-098 | N—Ph | N—Ph | b3 | H | b3 | b9 |
| 2-12-099 | N—Ph | N—Ph | H | H | H | b10 |
| 2-12-100 | N—Ph | N—Ph | t-bu | H | t-bu | b10 |
| 2-12-101 | N—Ph | N—Ph | methyl | H | methyl | b10 |
| 2-12-102 | N—Ph | N—Ph | b1 | H | b1 | b10 |
| 2-12-103 | N—Ph | N—Ph | b2 | H | b2 | b10 |
| 2-12-104 | N—Ph | N—Ph | b3 | H | b3 | b10 |
| 2-12-105 | N-tolyl | N-tolyl | H | H | H | H |
| 2-12-106 | N-tolyl | N-tolyl | t-bu | H | t-bu | H |
| 2-12-107 | N-tolyl | N-tolyl | methyl | H | methyl | H |
| 2-12-108 | N-tolyl | N-tolyl | t-bu | t-bu | t-bu | t-bu |
| 2-12-109 | N-tolyl | N-tolyl | methyl | methyl | methyl | methyl |
| 2-12-110 | N-tolyl | N-tolyl | b1 | H | b1 | H |
| 2-12-111 | N-tolyl | N-tolyl | b2 | H | b2 | H |
| 2-12-112 | N-tolyl | N-tolyl | b3 | H | b3 | H |
| 2-12-113 | N-tolyl | N-tolyl | H | H | H | a1 |
| 2-12-114 | N-tolyl | N-tolyl | t-bu | H | t-bu | a1 |
| 2-12-115 | N-tolyl | N-tolyl | methyl | H | methyl | a1 |
| 2-12-116 | N-tolyl | N-tolyl | b1 | H | b1 | a1 |
| 2-12-117 | N-tolyl | N-tolyl | b2 | H | b2 | a1 |
| 2-12-118 | N-tolyl | N-tolyl | b3 | H | b3 | a1 |
| 2-12-119 | N-tolyl | N-tolyl | H | H | H | a2 |
| 2-12-120 | N-tolyl | N-tolyl | t-bu | H | t-bu | a2 |
| 2-12-121 | N-tolyl | N-tolyl | methyl | H | methyl | a2 |
| 2-12-122 | N-tolyl | N-tolyl | b1 | H | b1 | a2 |
| 2-12-123 | N-tolyl | N-tolyl | b2 | H | b2 | a2 |
| 2-12-124 | N-tolyl | N-tolyl | b3 | H | b3 | a2 |
| 2-12-125 | N-tolyl | N-tolyl | H | H | H | a3 |
| 2-12-126 | N-tolyl | N-tolyl | t-bu | H | t-bu | a3 |
| 2-12-127 | N-tolyl | N-tolyl | methyl | H | methyl | a3 |
| 2-12-128 | N-tolyl | N-tolyl | b1 | H | b1 | a3 |
| 2-12-129 | N-tolyl | N-tolyl | b2 | H | b2 | a3 |
| 2-12-130 | N-tolyl | N-tolyl | b3 | H | b3 | a3 |
| 2-12-131 | N-tolyl | N-tolyl | H | H | H | a4 |
| 2-12-132 | N-tolyl | N-tolyl | t-bu | H | t-bu | a4 |
| 2-12-133 | N-tolyl | N-tolyl | methyl | H | methyl | a4 |
| 2-12-134 | N-tolyl | N-tolyl | b1 | H | b1 | a4 |
| 2-12-135 | N-tolyl | N-tolyl | b2 | H | b2 | a4 |
| 2-12-136 | N-tolyl | N-tolyl | b3 | H | b3 | a4 |
| 2-12-137 | N-tolyl | N-tolyl | H | H | H | a5 |
| 2-12-138 | N-tolyl | N-tolyl | t-bu | H | t-bu | a5 |
| 2-12-139 | N-tolyl | N-tolyl | methyl | H | methyl | a5 |
| 2-12-140 | N-tolyl | N-tolyl | b1 | H | b1 | a5 |
| 2-12-141 | N-tolyl | N-tolyl | b2 | H | b2 | a5 |
| 2-12-142 | N-tolyl | N-tolyl | b3 | H | b3 | a5 |
| 2-12-143 | N-tolyl | N-tolyl | H | H | H | a6 |
| 2-12-144 | N-tolyl | N-tolyl | t-bu | H | t-bu | a6 |
| 2-12-145 | N-tolyl | N-tolyl | methyl | H | methyl | a6 |
| 2-12-146 | N-tolyl | N-tolyl | b1 | H | b1 | a6 |
| 2-12-147 | N-tolyl | N-tolyl | b2 | H | b2 | a6 |
| 2-12-148 | N-tolyl | N-tolyl | b3 | H | b3 | a6 |
| 2-12-149 | N-tolyl | N-tolyl | H | H | H | b1 |
| 2-12-150 | N-tolyl | N-tolyl | t-bu | H | t-bu | b1 |
| 2-12-151 | N-tolyl | N-tolyl | methyl | H | methyl | b1 |
| 2-12-152 | N-tolyl | N-tolyl | b1 | H | b1 | b1 |
| 2-12-153 | N-tolyl | N-tolyl | b2 | H | b2 | b1 |
| 2-12-154 | N-tolyl | N-tolyl | b3 | H | b3 | b1 |
| 2-12-155 | N-tolyl | N-tolyl | H | H | H | b2 |
| 2-12-156 | N-tolyl | N-tolyl | t-bu | H | t-bu | b2 |
| 2-12-157 | N-tolyl | N-tolyl | methyl | H | methyl | b2 |
| 2-12-158 | N-tolyl | N-tolyl | b1 | H | b1 | b2 |
| 2-12-159 | N-tolyl | N-tolyl | b2 | H | b2 | b2 |
| 2-12-160 | N-tolyl | N-tolyl | b3 | H | b3 | b2 |
| 2-12-161 | N-tolyl | N-tolyl | H | H | H | b3 |
| 2-12-162 | N-tolyl | N-tolyl | t-bu | H | t-bu | b3 |
| 2-12-163 | N-tolyl | N-tolyl | methyl | H | methyl | b3 |
| 2-12-164 | N-tolyl | N-tolyl | b1 | H | b1 | b3 |
| 2-12-165 | N-tolyl | N-tolyl | b2 | H | b2 | b3 |
| 2-12-166 | N-tolyl | N-tolyl | b3 | H | b3 | b3 |
| 2-12-167 | N-tolyl | N-tolyl | H | H | H | b4 |
| 2-12-168 | N-tolyl | N-tolyl | t-bu | H | t-bu | b4 |
| 2-12-169 | N-tolyl | N-tolyl | methyl | H | methyl | b4 |
| 2-12-170 | N-tolyl | N-tolyl | b1 | H | b1 | b4 |
| 2-12-171 | N-tolyl | N-tolyl | b2 | H | b2 | b4 |
| 2-12-172 | N-tolyl | N-tolyl | b3 | H | b3 | b4 |
| 2-12-173 | N-tolyl | N-tolyl | H | H | H | b5 |
| 2-12-174 | N-tolyl | N-tolyl | t-bu | H | t-bu | b5 |
| 2-12-175 | N-tolyl | N-tolyl | methyl | H | methyl | b5 |
| 2-12-176 | N-tolyl | N-tolyl | b1 | H | b1 | b5 |
| 2-12-177 | N-tolyl | N-tolyl | b2 | H | b2 | b5 |
| 2-12-178 | N-tolyl | N-tolyl | b3 | H | b3 | b5 |
| 2-12-179 | N-tolyl | N-tolyl | H | H | H | b6 |
| 2-12-180 | N-tolyl | N-tolyl | t-bu | H | t-bu | b6 |
| 2-12-181 | N-tolyl | N-tolyl | methyl | H | methyl | b6 |
| 2-12-182 | N-tolyl | N-tolyl | b1 | H | b1 | b6 |
| 2-12-183 | N-tolyl | N-tolyl | b2 | H | b2 | b6 |
| 2-12-184 | N-tolyl | N-tolyl | b3 | H | b3 | b6 |
| 2-12-185 | N-tolyl | N-tolyl | H | H | H | b7 |
| 2-12-186 | N-tolyl | N-tolyl | t-bu | H | t-bu | b7 |
| 2-12-187 | N-tolyl | N-tolyl | methyl | H | methyl | b7 |
| 2-12-188 | N-tolyl | N-tolyl | b1 | H | b1 | b7 |
| 2-12-189 | N-tolyl | N-tolyl | b2 | H | b2 | b7 |
| 2-12-190 | N-tolyl | N-tolyl | b3 | H | b3 | b7 |
| 2-12-191 | N-tolyl | N-tolyl | H | H | H | b8 |
| 2-12-192 | N-tolyl | N-tolyl | t-bu | H | t-bu | b8 |
| 2-12-193 | N-tolyl | N-tolyl | methyl | H | methyl | b8 |
| 2-12-194 | N-tolyl | N-tolyl | b1 | H | b1 | b8 |
| 2-12-195 | N-tolyl | N-tolyl | b2 | H | b2 | b8 |
| 2-12-196 | N-tolyl | N-tolyl | b3 | H | b3 | b8 |
| 2-12-197 | N-tolyl | N-tolyl | H | H | H | b9 |
| 2-12-198 | N-tolyl | N-tolyl | t-bu | H | t-bu | b9 |
| 2-12-199 | N-tolyl | N-tolyl | methyl | H | methyl | b9 |
| 2-12-200 | N-tolyl | N-tolyl | b1 | H | b1 | b9 |
| 2-12-201 | N-tolyl | N-tolyl | b2 | H | b2 | b9 |
| 2-12-202 | N-tolyl | N-tolyl | b3 | H | b3 | b9 |
| 2-12-203 | N-tolyl | N-tolyl | H | H | H | b10 |
| 2-12-204 | N-tolyl | N-tolyl | t-bu | H | t-bu | b10 |
| 2-12-205 | N-tolyl | N-tolyl | methyl | H | methyl | b10 |
| 2-12-206 | N-tolyl | N-tolyl | b1 | H | b1 | b10 |
| 2-12-207 | N-tolyl | N-tolyl | b2 | H | b2 | b10 |
| 2-12-208 | N-tolyl | N-tolyl | b3 | H | b3 | b10 |
| 2-12-209 | N-Biph | N-Biph | H | H | H | H |
| 2-12-210 | N-Biph | N-Biph | t-bu | H | t-bu | H |
| 2-12-211 | N-Biph | N-Biph | methyl | H | methyl | H |
| 2-12-212 | N-Biph | N-Biph | t-bu | t-bu | t-bu | t-bu |
| 2-12-213 | N-Biph | N-Biph | methyl | methyl | methyl | methyl |
| 2-12-214 | N-Biph | N-Biph | b1 | H | b1 | H |
| 2-12-215 | N-Biph | N-Biph | b2 | H | b2 | H |
| 2-12-216 | N-Biph | N-Biph | b3 | H | b3 | H |
| 2-12-217 | N-Biph | N-Biph | H | H | H | a1 |
| 2-12-218 | N-Biph | N-Biph | t-bu | H | t-bu | a1 |
| 2-12-219 | N-Biph | N-Biph | methyl | H | methyl | a1 |
| 2-12-220 | N-Biph | N-Biph | b1 | H | b1 | a1 |
| 2-12-221 | N-Biph | N-Biph | b2 | H | b2 | a1 |
| 2-12-222 | N-Biph | N-Biph | b3 | H | b3 | a1 |
| 2-12-223 | N-Biph | N-Biph | H | H | H | a2 |
| 2-12-224 | N-Biph | N-Biph | t-bu | H | t-bu | a2 |
| 2-12-225 | N-Biph | N-Biph | methyl | H | methyl | a2 |
| 2-12-226 | N-Biph | N-Biph | b1 | H | b1 | a2 |
| 2-12-227 | N-Biph | N-Biph | b2 | H | b2 | a2 |
| 2-12-228 | N-Biph | N-Biph | b3 | H | b3 | a2 |
| 2-12-229 | N-Biph | N-Biph | H | H | H | a3 |
| 2-12-230 | N-Biph | N-Biph | t-bu | H | t-bu | a3 |
| 2-12-231 | N-Biph | N-Biph | methyl | H | methyl | a3 |
| 2-12-232 | N-Biph | N-Biph | b1 | H | b1 | a3 |
| 2-12-233 | N-Biph | N-Biph | b2 | H | b2 | a3 |
| 2-12-234 | N-Biph | N-Biph | b3 | H | b3 | a3 |
| 2-12-235 | N-Biph | N-Biph | H | H | H | a4 |
| 2-12-236 | N-Biph | N-Biph | t-bu | H | t-bu | a4 |
| 2-12-237 | N-Biph | N-Biph | methyl | H | methyl | a4 |
| 2-12-238 | N-Biph | N-Biph | b1 | H | b1 | a4 |
| 2-12-239 | N-Biph | N-Biph | b2 | H | b2 | a4 |
| 2-12-240 | N-Biph | N-Biph | b3 | H | b3 | a4 |
| 2-12-241 | N-Biph | N-Biph | H | H | H | a5 |
| 2-12-242 | N-Biph | N-Biph | t-bu | H | t-bu | a5 |
| 2-12-243 | N-Biph | N-Biph | methyl | H | methyl | a5 |
| 2-12-244 | N-Biph | N-Biph | b1 | H | b1 | a5 |
| 2-12-245 | N-Biph | N-Biph | b2 | H | b2 | a5 |
| 2-12-246 | N-Biph | N-Biph | b3 | H | b3 | a5 |
| 2-12-247 | N-Biph | N-Biph | H | H | H | a6 |
| 2-12-248 | N-Biph | N-Biph | t-bu | H | t-bu | a6 |
| 2-12-249 | N-Biph | N-Biph | methyl | H | methyl | a6 |
| 2-12-250 | N-Biph | N-Biph | b1 | H | b1 | a6 |
| 2-12-251 | N-Biph | N-Biph | b2 | H | b2 | a6 |
| 2-12-252 | N-Biph | N-Biph | b3 | H | b3 | a6 |
| 2-12-253 | N-Biph | N-Biph | H | H | H | b1 |
| 2-12-254 | N-Biph | N-Biph | t-bu | H | t-bu | b1 |
| 2-12-255 | N-Biph | N-Biph | methyl | H | methyl | b1 |
| 2-12-256 | N-Biph | N-Biph | b1 | H | b1 | b1 |
| 2-12-257 | N-Biph | N-Biph | b2 | H | b2 | b1 |
| 2-12-258 | N-Biph | N-Biph | b3 | H | b3 | b1 |
| 2-12-259 | N-Biph | N-Biph | H | H | H | b2 |
| 2-12-260 | N-Biph | N-Biph | t-bu | H | t-bu | b2 |
| 2-12-261 | N-Biph | N-Biph | methyl | H | methyl | b2 |
| 2-12-262 | N-Biph | N-Biph | b1 | H | b1 | b2 |
| 2-12-263 | N-Biph | N-Biph | b2 | H | b2 | b2 |
| 2-12-264 | N-Biph | N-Biph | b3 | H | b3 | b2 |
| 2-12-265 | N-Biph | N-Biph | H | H | H | b3 |
| 2-12-266 | N-Biph | N-Biph | t-bu | H | t-bu | b3 |
| 2-12-267 | N-Biph | N-Biph | methyl | H | methyl | b3 |
| 2-12-268 | N-Biph | N-Biph | b1 | H | b1 | b3 |
| 2-12-269 | N-Biph | N-Biph | b2 | H | b2 | b3 |
| 2-12-270 | N-Biph | N-Biph | b3 | H | b3 | b3 |
| 2-12-271 | N-Biph | N-Biph | H | H | H | b4 |
| 2-12-272 | N-Biph | N-Biph | t-bu | H | t-bu | b4 |
| 2-12-273 | N-Biph | N-Biph | methyl | H | methyl | b4 |
| 2-12-274 | N-Biph | N-Biph | b1 | H | b1 | b4 |
| 2-12-275 | N-Biph | N-Biph | b2 | H | b2 | b4 |
| 2-12-276 | N-Biph | N-Biph | b3 | H | b3 | b4 |
| 2-12-277 | N-Biph | N-Biph | H | H | H | b5 |
| 2-12-278 | N-Biph | N-Biph | t-bu | H | t-bu | b5 |
| 2-12-279 | N-Biph | N-Biph | methyl | H | methyl | b5 |
| 2-12-280 | N-Biph | N-Biph | b1 | H | b1 | b5 |
| 2-12-281 | N-Biph | N-Biph | b2 | H | b2 | b5 |
| 2-12-282 | N-Biph | N-Biph | b3 | H | b3 | b5 |
| 2-12-283 | N-Biph | N-Biph | H | H | H | b6 |
| 2-12-284 | N-Biph | N-Biph | t-bu | H | t-bu | b6 |
| 2-12-285 | N-Biph | N-Biph | methyl | H | methyl | b6 |
| 2-12-286 | N-Biph | N-Biph | b1 | H | b1 | b6 |
| 2-12-287 | N-Biph | N-Biph | b2 | H | b2 | b6 |
| 2-12-288 | N-Biph | N-Biph | b3 | H | b3 | b6 |
| 2-12-289 | N-Biph | N-Biph | H | H | H | b7 |
| 2-12-290 | N-Biph | N-Biph | t-bu | H | t-bu | b7 |
| 2-12-291 | N-Biph | N-Biph | methyl | H | methyl | b7 |
| 2-12-292 | N-Biph | N-Biph | b1 | H | b1 | b7 |
| 2-12-293 | N-Biph | N-Biph | b2 | H | b2 | b7 |
| 2-12-294 | N-Biph | N-Biph | b3 | H | b3 | b7 |
| 2-12-295 | N-Biph | N-Biph | H | H | H | b8 |
| 2-12-296 | N-Biph | N-Biph | t-bu | H | t-bu | b8 |
| 2-12-297 | N-Biph | N-Biph | methyl | H | methyl | b8 |
| 2-12-298 | N-Biph | N-Biph | b1 | H | b1 | b8 |
| 2-12-299 | N-Biph | N-Biph | b2 | H | b2 | b8 |
| 2-12-300 | N-Biph | N-Biph | b3 | H | b3 | b8 |
| 2-12-301 | N-Biph | N-Biph | H | H | H | b9 |
| 2-12-302 | N-Biph | N-Biph | t-bu | H | t-bu | b9 |
| 2-12-303 | N-Biph | N-Biph | methyl | H | methyl | b9 |
| 2-12-304 | N-Biph | N-Biph | b1 | H | b1 | b9 |
| 2-12-305 | N-Biph | N-Biph | b2 | H | b2 | b9 |
| 2-12-306 | N-Biph | N-Biph | b3 | H | b3 | b9 |
| 2-12-307 | N-Biph | N-Biph | H | H | H | b10 |
| 2-12-308 | N-Biph | N-Biph | t-bu | H | t-bu | b10 |
| 2-12-309 | N-Biph | N-Biph | methyl | H | methyl | b10 |
| 2-12-310 | N-Biph | N-Biph | b1 | H | b1 | b10 |
| 2-12-311 | N-Biph | N-Biph | b2 | H | b2 | b10 |
| 2-12-312 | N-Biph | N-Biph | b3 | H | b3 | b10 |
| 2-12-313 | O | N—Ph | H | H | H | H |
| 2-12-314 | O | N—Ph | t-bu | H | t-bu | H |
| 2-12-315 | O | N—Ph | methyl | H | methyl | H |
| 2-12-316 | O | N—Ph | t-bu | t-bu | t-bu | t-bu |
| 2-12-317 | O | N—Ph | methyl | methyl | methyl | methyl |
| 2-12-318 | O | N—Ph | b1 | H | b1 | H |
| 2-12-319 | O | N—Ph | b2 | H | b2 | H |
| 2-12-320 | O | N—Ph | b3 | H | b3 | H |
| 2-12-321 | O | N—Ph | H | H | H | a1 |
| 2-12-322 | O | N—Ph | t-bu | H | t-bu | a1 |
| 2-12-323 | O | N—Ph | methyl | H | methyl | a1 |
| 2-12-324 | O | N—Ph | b1 | H | b1 | a1 |
| 2-12-325 | O | N—Ph | b2 | H | b2 | a1 |
| 2-12-326 | O | N—Ph | b3 | H | b3 | a1 |
| 2-12-327 | O | N—Ph | H | H | H | a2 |
| 2-12-328 | O | N—Ph | t-bu | H | t-bu | a2 |
| 2-12-329 | O | N—Ph | methyl | H | methyl | a2 |
| 2-12-330 | O | N—Ph | b1 | H | b1 | a2 |
| 2-12-331 | O | N—Ph | b2 | H | b2 | a2 |
| 2-12-332 | O | N—Ph | b3 | H | b3 | a2 |
| 2-12-333 | O | N—Ph | H | H | H | a3 |
| 2-12-334 | O | N—Ph | t-bu | H | t-bu | a3 |
| 2-12-335 | O | N—Ph | methyl | H | methyl | a3 |
| 2-12-336 | O | N—Ph | b1 | H | b1 | a3 |
| 2-12-337 | O | N—Ph | b2 | H | b2 | a3 |
| 2-12-338 | O | N—Ph | b3 | H | b3 | a3 |
| 2-12-339 | O | N—Ph | H | H | H | a4 |
| 2-12-340 | O | N—Ph | t-bu | H | t-bu | a4 |
| 2-12-341 | O | N—Ph | methyl | H | methyl | a4 |
| 2-12-342 | O | N—Ph | b1 | H | b1 | a4 |
| 2-12-343 | O | N—Ph | b2 | H | b2 | a4 |
| 2-12-344 | O | N—Ph | b3 | H | b3 | a4 |
| 2-12-345 | O | N—Ph | H | H | H | a5 |
| 2-12-346 | O | N—Ph | t-bu | H | t-bu | a5 |
| 2-12-347 | O | N—Ph | methyl | H | methyl | a5 |
| 2-12-348 | O | N—Ph | b1 | H | b1 | a5 |
| 2-12-349 | O | N—Ph | b2 | H | b2 | a5 |
| 2-12-350 | O | N—Ph | b3 | H | b3 | a5 |
| 2-12-351 | O | N—Ph | H | H | H | a6 |
| 2-12-352 | O | N—Ph | t-bu | H | t-bu | a6 |
| 2-12-353 | O | N—Ph | methyl | H | methyl | a6 |
| 2-12-354 | O | N—Ph | b1 | H | b1 | a6 |
| 2-12-355 | O | N—Ph | b2 | H | b2 | a6 |
| 2-12-356 | O | N—Ph | b3 | H | b3 | a6 |
| 2-12-357 | O | N—Ph | H | H | H | b1 |
| 2-12-358 | O | N—Ph | t-bu | H | t-bu | b1 |
| 2-12-359 | O | N—Ph | methyl | H | methyl | b1 |
| 2-12-360 | O | N—Ph | b1 | H | b1 | b1 |
| 2-12-361 | O | N—Ph | b2 | H | b2 | b1 |
| 2-12-362 | O | N—Ph | b3 | H | b3 | b1 |
| 2-12-363 | O | N—Ph | H | H | H | b2 |
| 2-12-364 | O | N—Ph | t-bu | H | t-bu | b2 |
| 2-12-365 | O | N—Ph | methyl | H | methyl | b2 |
| 2-12-366 | O | N—Ph | b1 | H | b1 | b2 |
| 2-12-367 | O | N—Ph | b2 | H | b2 | b2 |
| 2-12-368 | O | N—Ph | b3 | H | b3 | b2 |
| 2-12-369 | O | N—Ph | H | H | H | b3 |
| 2-12-370 | O | N—Ph | t-bu | H | t-bu | b3 |
| 2-12-371 | O | N—Ph | methyl | H | methyl | b3 |
| 2-12-372 | O | N—Ph | b1 | H | b1 | b3 |
| 2-12-373 | O | N—Ph | b2 | H | b2 | b3 |
| 2-12-374 | O | N—Ph | b3 | H | b3 | b3 |
| 2-12-375 | O | N—Ph | H | H | H | b4 |
| 2-12-376 | O | N—Ph | t-bu | H | t-bu | b4 |
| 2-12-377 | O | N—Ph | methyl | H | methyl | b4 |
| 2-12-378 | O | N—Ph | b1 | H | b1 | b4 |
| 2-12-379 | O | N—Ph | b2 | H | b2 | b4 |
| 2-12-380 | O | N—Ph | b3 | H | b3 | b4 |
| 2-12-381 | O | N—Ph | H | H | H | b5 |
| 2-12-382 | O | N—Ph | t-bu | H | t-bu | b5 |
| 2-12-383 | O | N—Ph | methyl | H | methyl | b5 |
| 2-12-384 | O | N—Ph | b1 | H | b1 | b5 |
| 2-12-385 | O | N—Ph | b2 | H | b2 | b5 |
| 2-12-386 | O | N—Ph | b3 | H | b3 | b5 |
| 2-12-387 | O | N—Ph | H | H | H | b6 |
| 2-12-388 | O | N—Ph | t-bu | H | t-bu | b6 |
| 2-12-389 | O | N—Ph | methyl | H | methyl | b6 |
| 2-12-390 | O | N—Ph | b1 | H | b1 | b6 |
| 2-12-391 | O | N—Ph | b2 | H | b2 | b6 |
| 2-12-392 | O | N—Ph | b3 | H | b3 | b6 |
| 2-12-393 | O | N—Ph | H | H | H | b7 |
| 2-12-394 | O | N—Ph | t-bu | H | t-bu | b7 |
| 2-12-395 | O | N—Ph | methyl | H | methyl | b7 |
| 2-12-396 | O | N—Ph | b1 | H | b1 | b7 |
| 2-12-397 | O | N—Ph | b2 | H | b2 | b7 |
| 2-12-398 | O | N—Ph | b3 | H | b3 | b7 |
| 2-12-399 | O | N—Ph | H | H | H | b8 |
| 2-12-400 | O | N—Ph | t-bu | H | t-bu | b8 |
| 2-12-401 | O | N—Ph | methyl | H | methyl | b8 |
| 2-12-402 | O | N—Ph | b1 | H | b1 | b8 |
| 2-12-403 | O | N—Ph | b2 | H | b2 | b8 |
| 2-12-404 | O | N—Ph | b3 | H | b3 | b8 |
| 2-12-405 | O | N—Ph | H | H | H | b9 |
| 2-12-406 | O | N—Ph | t-bu | H | t-bu | b9 |
| 2-12-407 | O | N—Ph | methyl | H | methyl | b9 |
| 2-12-408 | O | N—Ph | b1 | H | b1 | b9 |
| 2-12-409 | O | N—Ph | b2 | H | b2 | b9 |
| 2-12-410 | O | N—Ph | b3 | H | b3 | b9 |
| 2-12-411 | O | N—Ph | H | H | H | b10 |
| 2-12-412 | O | N—Ph | t-bu | H | t-bu | b10 |
| 2-12-413 | O | N—Ph | methyl | H | methyl | b10 |
| 2-12-414 | O | N—Ph | b1 | H | b1 | b10 |
| 2-12-415 | O | N—Ph | b2 | H | b2 | b10 |
| 2-12-416 | O | N—Ph | b3 | H | b3 | b10 |
| 2-12-417 | S | N—Ph | H | H | H | H |
| 2-12-418 | S | N—Ph | t-bu | H | t-bu | H |
| 2-12-419 | S | N—Ph | methyl | H | methyl | H |
| 2-12-420 | S | N—Ph | t-bu | t-bu | t-bu | t-bu |
| 2-12-421 | S | N—Ph | methyl | methyl | methyl | methyl |
| 2-12-422 | S | N—Ph | b1 | H | b1 | H |
| 2-12-423 | S | N—Ph | b2 | H | b2 | H |
| 2-12-424 | S | N—Ph | b3 | H | b3 | H |
| 2-12-425 | S | N—Ph | H | H | H | a1 |
| 2-12-426 | S | N—Ph | t-bu | H | t-bu | a1 |
| 2-12-427 | S | N—Ph | methyl | H | methyl | a1 |
| 2-12-428 | S | N—Ph | b1 | H | b1 | a1 |
| 2-12-429 | S | N—Ph | b2 | H | b2 | a1 |
| 2-12-430 | S | N—Ph | b3 | H | b3 | a1 |
| 2-12-431 | S | N—Ph | H | H | H | a2 |
| 2-12-432 | S | N—Ph | t-bu | H | t-bu | a2 |
| 2-12-433 | S | N—Ph | methyl | H | methyl | a2 |
| 2-12-434 | S | N—Ph | b1 | H | b1 | a2 |
| 2-12-435 | S | N—Ph | b2 | H | b2 | a2 |
| 2-12-436 | S | N—Ph | b3 | H | b3 | a2 |
| 2-12-437 | S | N—Ph | H | H | H | a3 |
| 2-12-438 | S | N—Ph | t-bu | H | t-bu | a3 |
| 2-12-439 | S | N—Ph | methyl | H | methyl | a3 |
| 2-12-440 | S | N—Ph | b1 | H | b1 | a3 |
| 2-12-441 | S | N—Ph | b2 | H | b2 | a3 |
| 2-12-442 | S | N—Ph | b3 | H | b3 | a3 |
| 2-12-443 | S | N—Ph | H | H | H | a4 |
| 2-12-444 | S | N—Ph | t-bu | H | t-bu | a4 |
| 2-12-445 | S | N—Ph | methyl | H | methyl | a4 |
| 2-12-446 | S | N—Ph | b1 | H | b1 | a4 |
| 2-12-447 | S | N—Ph | b2 | H | b2 | a4 |
| 2-12-448 | S | N—Ph | b3 | H | b3 | a4 |
| 2-12-449 | S | N—Ph | H | H | H | a5 |
| 2-12-450 | S | N—Ph | t-bu | H | t-bu | a5 |
| 2-12-451 | S | N—Ph | methyl | H | methyl | a5 |
| 2-12-452 | S | N—Ph | b1 | H | b1 | a5 |
| 2-12-453 | S | N—Ph | b2 | H | b2 | a5 |
| 2-12-454 | S | N—Ph | b3 | H | b3 | a5 |
| 2-12-455 | S | N—Ph | H | H | H | a6 |
| 2-12-456 | S | N—Ph | t-bu | H | t-bu | a6 |
| 2-12-457 | S | N—Ph | methyl | H | methyl | a6 |
| 2-12-458 | S | N—Ph | b1 | H | b1 | a6 |
| 2-12-459 | S | N—Ph | b2 | H | b2 | a6 |
| 2-12-460 | S | N—Ph | b3 | H | b3 | a6 |
| 2-12-461 | S | N—Ph | H | H | H | b1 |
| 2-12-462 | S | N—Ph | t-bu | H | t-bu | b1 |
| 2-12-463 | S | N—Ph | methyl | H | methyl | b1 |
| 2-12-464 | S | N—Ph | b1 | H | b1 | b1 |
| 2-12-465 | S | N—Ph | b2 | H | b2 | b1 |
| 2-12-466 | S | N—Ph | b3 | H | b3 | b1 |
| 2-12-467 | S | N—Ph | H | H | H | b2 |
| 2-12-468 | S | N—Ph | t-bu | H | t-bu | b2 |
| 2-12-469 | S | N—Ph | methyl | H | methyl | b2 |
| 2-12-470 | S | N—Ph | b1 | H | b1 | b2 |
| 2-12-471 | S | N—Ph | b2 | H | b2 | b2 |
| 2-12-472 | S | N—Ph | b3 | H | b3 | b2 |
| 2-12-473 | S | N—Ph | H | H | H | b3 |
| 2-12-474 | S | N—Ph | t-bu | H | t-bu | b3 |
| 2-12-475 | S | N—Ph | methyl | H | methyl | b3 |
| 2-12-476 | S | N—Ph | b1 | H | b1 | b3 |
| 2-12-477 | S | N—Ph | b2 | H | b2 | b3 |
| 2-12-478 | S | N—Ph | b3 | H | b3 | b3 |
| 2-12-479 | S | N—Ph | H | H | H | b4 |
| 2-12-480 | S | N—Ph | t-bu | H | t-bu | b4 |
| 2-12-481 | S | N—Ph | methyl | H | methyl | b4 |
| 2-12-482 | S | N—Ph | b1 | H | b1 | b4 |
| 2-12-483 | S | N—Ph | b2 | H | b2 | b4 |
| 2-12-484 | S | N—Ph | b3 | H | b3 | b4 |
| 2-12-485 | S | N—Ph | H | H | H | b5 |
| 2-12-486 | S | N—Ph | t-bu | H | t-bu | b5 |
| 2-12-487 | S | N—Ph | methyl | H | methyl | b5 |
| 2-12-488 | S | N—Ph | b1 | H | b1 | b5 |
| 2-12-489 | S | N—Ph | b2 | H | b2 | b5 |
| 2-12-490 | S | N—Ph | b3 | H | b3 | b5 |
| 2-12-491 | S | N—Ph | H | H | H | b6 |
| 2-12-492 | S | N—Ph | t-bu | H | t-bu | b6 |
| 2-12-493 | S | N—Ph | methyl | H | methyl | b6 |
| 2-12-494 | S | N—Ph | b1 | H | b1 | b6 |
| 2-12-495 | S | N—Ph | b2 | H | b2 | b6 |
| 2-12-496 | S | N—Ph | b3 | H | b3 | b6 |
| 2-12-497 | S | N—Ph | H | H | H | b7 |
| 2-12-498 | S | N—Ph | t-bu | H | t-bu | b7 |
| 2-12-499 | S | N—Ph | methyl | H | methyl | b7 |
| 2-12-500 | S | N—Ph | b1 | H | b1 | b7 |
| 2-12-501 | S | N—Ph | b2 | H | b2 | b7 |
| 2-12-502 | S | N—Ph | b3 | H | b3 | b7 |
| 2-12-503 | S | N—Ph | H | H | H | b8 |
| 2-12-504 | S | N—Ph | t-bu | H | t-bu | b8 |
| 2-12-505 | S | N—Ph | methyl | H | methyl | b8 |
| 2-12-506 | S | N—Ph | b1 | H | b1 | b8 |
| 2-12-507 | S | N—Ph | b2 | H | b2 | b8 |
| 2-12-508 | S | N—Ph | b3 | H | b3 | b8 |
| 2-12-509 | S | N—Ph | H | H | H | b9 |
| 2-12-510 | S | N—Ph | t-bu | H | t-bu | b9 |
| 2-12-511 | S | N—Ph | methyl | H | methyl | b9 |
| 2-12-512 | S | N—Ph | b1 | H | b1 | b9 |
| 2-12-513 | S | N—Ph | b2 | H | b2 | b9 |
| 2-12-514 | S | N—Ph | b3 | H | b3 | b9 |
| 2-12-515 | S | N—Ph | H | H | H | b10 |
| 2-12-516 | S | N—Ph | t-bu | H | t-bu | b10 |
| 2-12-517 | S | N—Ph | methyl | H | methyl | b10 |
| 2-12-518 | S | N—Ph | b1 | H | b1 | b10 |
| 2-12-519 | S | N—Ph | b2 | H | b2 | b10 |
| 2-12-520 | S | N—Ph | b3 | H | b3 | b10 |

### Group 2-13

The compounds are represented by the formula 2-13 below, wherein CY₁ is a benzene group, X₁ is B, and Y1, Y2, R2a, R2c, R3a, (R1)a1 are each shown in the table 13 below,

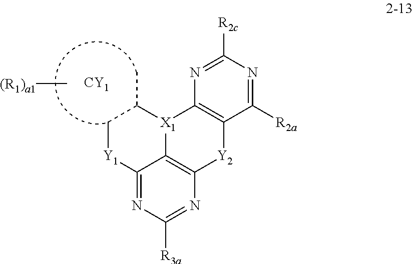

TABLE 13

| Compound No. | Y1 | Y2 | R2a | R2c | R3a | (R1)a1 |
|---|---|---|---|---|---|---|
| 2-13-001 | N—Ph | N—Ph | H | H | H | H |
| 2-13-002 | N—Ph | N—Ph | t-bu | H | t-bu | H |
| 2-13-003 | N—Ph | N—Ph | methyl | H | methyl | H |
| 2-13-004 | N—Ph | N—Ph | t-bu | t-bu | t-bu | t-bu |
| 2-13-005 | N—Ph | N—Ph | methyl | methyl | methyl | methyl |
| 2-13-006 | N—Ph | N—Ph | b1 | H | b1 | H |
| 2-13-007 | N—Ph | N—Ph | b2 | H | b2 | H |
| 2-13-008 | N—Ph | N—Ph | b3 | H | b3 | H |
| 2-13-009 | N—Ph | N—Ph | H | H | H | a1 |
| 2-13-010 | N—Ph | N—Ph | t-bu | t-bu | t-bu | a1 |
| 2-13-011 | N—Ph | N—Ph | methyl | methyl | methyl | a1 |
| 2-13-012 | N—Ph | N—Ph | b1 | b1 | b1 | a1 |
| 2-13-013 | N—Ph | N—Ph | b2 | b2 | b2 | a1 |
| 2-13-014 | N—Ph | N—Ph | b3 | b3 | b3 | a1 |
| 2-13-015 | N—Ph | N—Ph | H | H | H | a2 |
| 2-13-016 | N—Ph | N—Ph | t-bu | t-bu | t-bu | a2 |
| 2-13-017 | N—Ph | N—Ph | methyl | methyl | methyl | a2 |
| 2-13-018 | N—Ph | N—Ph | b1 | b1 | b1 | a2 |
| 2-13-019 | N—Ph | N—Ph | b2 | b2 | b2 | a2 |
| 2-13-020 | N—Ph | N—Ph | b3 | b3 | b3 | a2 |
| 2-13-021 | N—Ph | N—Ph | H | H | H | a3 |
| 2-13-022 | N—Ph | N—Ph | t-bu | t-bu | t-bu | a3 |
| 2-13-023 | N—Ph | N—Ph | methyl | methyl | methyl | a3 |
| 2-13-024 | N—Ph | N—Ph | b1 | b1 | b1 | a3 |
| 2-13-025 | N—Ph | N—Ph | b2 | b2 | b2 | a3 |
| 2-13-026 | N—Ph | N—Ph | b3 | b3 | b3 | a3 |
| 2-13-027 | N—Ph | N—Ph | H | H | H | a4 |
| 2-13-028 | N—Ph | N—Ph | t-bu | t-bu | t-bu | a4 |
| 2-13-029 | N—Ph | N—Ph | methyl | methyl | methyl | a4 |
| 2-13-030 | N—Ph | N—Ph | b1 | b1 | b1 | a4 |
| 2-13-031 | N—Ph | N—Ph | b2 | b2 | b2 | a4 |
| 2-13-032 | N—Ph | N—Ph | b3 | b3 | b3 | a4 |
| 2-13-033 | N—Ph | N—Ph | H | H | H | a5 |
| 2-13-034 | N—Ph | N—Ph | t-bu | t-bu | t-bu | a5 |
| 2-13-035 | N—Ph | N—Ph | methyl | methyl | methyl | a5 |
| 2-13-036 | N—Ph | N—Ph | b1 | b1 | b1 | a5 |
| 2-13-037 | N—Ph | N—Ph | b2 | b2 | b2 | a5 |
| 2-13-038 | N—Ph | N—Ph | b3 | b3 | b3 | a5 |
| 2-13-039 | N—Ph | N—Ph | H | H | H | a6 |
| 2-13-040 | N—Ph | N—Ph | t-bu | t-bu | t-bu | a6 |
| 2-13-041 | N—Ph | N—Ph | methyl | methyl | methyl | a6 |
| 2-13-042 | N—Ph | N—Ph | b1 | b1 | b1 | a6 |
| 2-13-043 | N—Ph | N—Ph | b2 | b2 | b2 | a6 |
| 2-13-044 | N—Ph | N—Ph | b3 | b3 | b3 | a6 |
| 2-13-045 | N—Ph | N—Ph | H | H | H | b1 |
| 2-13-046 | N—Ph | N—Ph | t-bu | t-bu | t-bu | b1 |
| 2-13-047 | N—Ph | N—Ph | methyl | methyl | methyl | b1 |
| 2-13-048 | N—Ph | N—Ph | b1 | b1 | b1 | b1 |
| 2-13-049 | N—Ph | N—Ph | b2 | b2 | b2 | b1 |
| 2-13-050 | N—Ph | N—Ph | b3 | b3 | b3 | b1 |
| 2-13-051 | N—Ph | N—Ph | H | H | H | b2 |
| 2-13-052 | N—Ph | N—Ph | t-bu | t-bu | t-bu | b2 |
| 2-13-053 | N—Ph | N—Ph | methyl | methyl | methyl | b2 |
| 2-13-054 | N—Ph | N—Ph | b1 | b1 | b1 | b2 |
| 2-13-055 | N—Ph | N—Ph | b2 | b2 | b2 | b2 |
| 2-13-056 | N—Ph | N—Ph | b3 | b3 | b3 | b2 |
| 2-13-057 | N—Ph | N—Ph | H | H | H | b3 |
| 2-13-058 | N—Ph | N—Ph | t-bu | t-bu | t-bu | b3 |
| 2-13-059 | N—Ph | N—Ph | methyl | methyl | methyl | b3 |
| 2-13-060 | N—Ph | N—Ph | b1 | b1 | b1 | b3 |
| 2-13-061 | N—Ph | N—Ph | b2 | b2 | b2 | b3 |
| 2-13-062 | N—Ph | N—Ph | b3 | b3 | b3 | b3 |
| 2-13-063 | N—Ph | N—Ph | H | H | H | b4 |
| 2-13-064 | N—Ph | N—Ph | t-bu | t-bu | t-bu | b4 |
| 2-13-065 | N—Ph | N—Ph | methyl | methyl | methyl | b4 |
| 2-13-066 | N—Ph | N—Ph | b1 | b1 | b1 | b4 |
| 2-13-067 | N—Ph | N—Ph | b2 | b2 | b2 | b4 |
| 2-13-068 | N—Ph | N—Ph | b3 | b3 | b3 | b4 |
| 2-13-069 | N—Ph | N—Ph | H | H | H | b5 |
| 2-13-070 | N—Ph | N—Ph | t-bu | t-bu | t-bu | b5 |
| 2-13-071 | N—Ph | N—Ph | methyl | methyl | methyl | b5 |
| 2-13-072 | N—Ph | N—Ph | b1 | b1 | b1 | b5 |
| 2-13-073 | N—Ph | N—Ph | b2 | b2 | b2 | b5 |
| 2-13-074 | N—Ph | N—Ph | b3 | b3 | b3 | b5 |
| 2-13-075 | N—Ph | N—Ph | H | H | H | b6 |
| 2-13-076 | N—Ph | N—Ph | t-bu | t-bu | t-bu | b6 |
| 2-13-077 | N—Ph | N—Ph | methyl | methyl | methyl | b6 |
| 2-13-078 | N—Ph | N—Ph | b1 | b1 | b1 | b6 |
| 2-13-079 | N—Ph | N—Ph | b2 | b2 | b2 | b6 |
| 2-13-080 | N—Ph | N—Ph | b3 | b3 | b3 | b6 |
| 2-13-081 | N—Ph | N—Ph | H | H | H | b7 |
| 2-13-082 | N—Ph | N—Ph | t-bu | t-bu | t-bu | b7 |
| 2-13-083 | N—Ph | N—Ph | methyl | methyl | methyl | b7 |
| 2-13-084 | N—Ph | N—Ph | b1 | b1 | b1 | b7 |
| 2-13-085 | N—Ph | N—Ph | b2 | b2 | b2 | b7 |
| 2-13-086 | N—Ph | N—Ph | b3 | b3 | b3 | b7 |
| 2-13-087 | N—Ph | N—Ph | H | H | H | b8 |
| 2-13-088 | N—Ph | N—Ph | t-bu | t-bu | t-bu | b8 |
| 2-13-089 | N—Ph | N—Ph | methyl | methyl | methyl | b8 |
| 2-13-090 | N—Ph | N—Ph | b1 | b1 | b1 | b8 |
| 2-13-091 | N—Ph | N—Ph | b2 | b2 | b2 | b8 |
| 2-13-092 | N—Ph | N—Ph | b3 | b3 | b3 | b8 |
| 2-13-093 | N—Ph | N—Ph | H | H | H | b9 |
| 2-13-094 | N—Ph | N—Ph | t-bu | t-bu | t-bu | b9 |
| 2-13-095 | N—Ph | N—Ph | methyl | methyl | methyl | b9 |
| 2-13-096 | N—Ph | N—Ph | b1 | b1 | b1 | b9 |
| 2-13-097 | N—Ph | N—Ph | b2 | b2 | b2 | b9 |
| 2-13-098 | N—Ph | N—Ph | b3 | b3 | b3 | b9 |
| 2-13-099 | N—Ph | N—Ph | H | H | H | b10 |
| 2-13-100 | N—Ph | N—Ph | t-bu | t-bu | t-bu | b10 |
| 2-13-101 | N—Ph | N—Ph | methyl | methyl | methyl | b10 |
| 2-13-102 | N—Ph | N—Ph | b1 | b1 | b1 | b10 |
| 2-13-103 | N—Ph | N—Ph | b2 | b2 | b2 | b10 |
| 2-13-104 | N—Ph | N—Ph | b3 | b3 | b3 | b10 |
| 2-13-105 | N-tolyl | N-tolyl | H | H | H | H |
| 2-13-106 | N-tolyl | N-tolyl | t-bu | H | t-bu | H |
| 2-13-107 | N-tolyl | N-tolyl | methyl | H | methyl | H |
| 2-13-108 | N-tolyl | N-tolyl | t-bu | t-bu | t-bu | t-bu |
| 2-13-109 | N-tolyl | N-tolyl | methyl | methyl | methyl | methyl |
| 2-13-110 | N-tolyl | N-tolyl | b1 | H | b1 | H |
| 2-13-111 | N-tolyl | N-tolyl | b2 | H | b2 | H |
| 2-13-112 | N-tolyl | N-tolyl | b3 | H | b3 | H |
| 2-13-113 | N-tolyl | N-tolyl | H | H | H | a1 |
| 2-13-114 | N-tolyl | N-tolyl | t-bu | t-bu | t-bu | a1 |
| 2-13-115 | N-tolyl | N-tolyl | methyl | methyl | methyl | a1 |
| 2-13-116 | N-tolyl | N-tolyl | b1 | b1 | b1 | a1 |
| 2-13-117 | N-tolyl | N-tolyl | b2 | b2 | b2 | a1 |
| 2-13-118 | N-tolyl | N-tolyl | b3 | b3 | b3 | a1 |
| 2-13-119 | N-tolyl | N-tolyl | H | H | H | a2 |
| 2-13-120 | N-tolyl | N-tolyl | t-bu | t-bu | t-bu | a2 |
| 2-13-121 | N-tolyl | N-tolyl | methyl | methyl | methyl | a2 |
| 2-13-122 | N-tolyl | N-tolyl | b1 | b1 | b1 | a2 |
| 2-13-123 | N-tolyl | N-tolyl | b2 | b2 | b2 | a2 |
| 2-13-124 | N-tolyl | N-tolyl | b3 | b3 | b3 | a2 |
| 2-13-125 | N-tolyl | N-tolyl | H | H | H | a3 |
| 2-13-126 | N-tolyl | N-tolyl | t-bu | t-bu | t-bu | a3 |
| 2-13-127 | N-tolyl | N-tolyl | methyl | methyl | methyl | a3 |
| 2-13-128 | N-tolyl | N-tolyl | b1 | b1 | b1 | a3 |
| 2-13-129 | N-tolyl | N-tolyl | b2 | b2 | b2 | a3 |
| 2-13-130 | N-tolyl | N-tolyl | b3 | b3 | b3 | a3 |
| 2-13-131 | N-tolyl | N-tolyl | H | H | H | a4 |
| 2-13-132 | N-tolyl | N-tolyl | t-bu | t-bu | t-bu | a4 |
| 2-13-133 | N-tolyl | N-tolyl | methyl | methyl | methyl | a4 |
| 2-13-134 | N-tolyl | N-tolyl | b1 | b1 | b1 | a4 |
| 2-13-135 | N-tolyl | N-tolyl | b2 | b2 | b2 | a4 |
| 2-13-136 | N-tolyl | N-tolyl | b3 | b3 | b3 | a4 |
| 2-13-137 | N-tolyl | N-tolyl | H | H | H | a5 |
| 2-13-138 | N-tolyl | N-tolyl | t-bu | t-bu | t-bu | a5 |
| 2-13-139 | N-tolyl | N-tolyl | methyl | methyl | methyl | a5 |
| 2-13-140 | N-tolyl | N-tolyl | b1 | b1 | b1 | a5 |
| 2-13-141 | N-tolyl | N-tolyl | b2 | b2 | b2 | a5 |
| 2-13-142 | N-tolyl | N-tolyl | b3 | b3 | b3 | a5 |
| 2-13-143 | N-tolyl | N-tolyl | H | H | H | a6 |
| 2-13-144 | N-tolyl | N-tolyl | t-bu | t-bu | t-bu | a6 |
| 2-13-145 | N-tolyl | N-tolyl | methyl | methyl | methyl | a6 |
| 2-13-146 | N-tolyl | N-tolyl | b1 | b1 | b1 | a6 |
| 2-13-147 | N-tolyl | N-tolyl | b2 | b2 | b2 | a6 |
| 2-13-148 | N-tolyl | N-tolyl | b3 | b3 | b3 | a6 |
| 2-13-149 | N-tolyl | N-tolyl | H | H | H | b1 |
| 2-13-150 | N-tolyl | N-tolyl | t-bu | t-bu | t-bu | b1 |
| 2-13-151 | N-tolyl | N-tolyl | methyl | methyl | methyl | b1 |
| 2-13-152 | N-tolyl | N-tolyl | b1 | b1 | b1 | b1 |
| 2-13-153 | N-tolyl | N-tolyl | b2 | b2 | b2 | b1 |
| 2-13-154 | N-tolyl | N-tolyl | b3 | b3 | b3 | b1 |
| 2-13-155 | N-tolyl | N-tolyl | H | H | H | b2 |
| 2-13-156 | N-tolyl | N-tolyl | t-bu | t-bu | t-bu | b2 |
| 2-13-157 | N-tolyl | N-tolyl | methyl | methyl | methyl | b2 |
| 2-13-158 | N-tolyl | N-tolyl | b1 | b1 | b1 | b2 |
| 2-13-159 | N-tolyl | N-tolyl | b2 | b2 | b2 | b2 |
| 2-13-160 | N-tolyl | N-tolyl | b3 | b3 | b3 | b2 |
| 2-13-161 | N-tolyl | N-tolyl | H | H | H | b3 |
| 2-13-162 | N-tolyl | N-tolyl | t-bu | t-bu | t-bu | b3 |
| 2-13-163 | N-tolyl | N-tolyl | methyl | methyl | methyl | b3 |
| 2-13-164 | N-tolyl | N-tolyl | b1 | b1 | b1 | b3 |
| 2-13-165 | N-tolyl | N-tolyl | b2 | b2 | b2 | b3 |
| 2-13-166 | N-tolyl | N-tolyl | b3 | b3 | b3 | b3 |
| 2-13-167 | N-tolyl | N-tolyl | H | H | H | b4 |
| 2-13-168 | N-tolyl | N-tolyl | t-bu | t-bu | t-bu | b4 |
| 2-13-169 | N-tolyl | N-tolyl | methyl | methyl | methyl | b4 |
| 2-13-170 | N-tolyl | N-tolyl | b1 | b1 | b1 | b4 |
| 2-13-171 | N-tolyl | N-tolyl | b2 | b2 | b2 | b4 |
| 2-13-172 | N-tolyl | N-tolyl | b3 | b3 | b3 | b4 |
| 2-13-173 | N-tolyl | N-tolyl | H | H | H | b5 |
| 2-13-174 | N-tolyl | N-tolyl | t-bu | t-bu | t-bu | b5 |
| 2-13-175 | N-tolyl | N-tolyl | methyl | methyl | methyl | b5 |
| 2-13-176 | N-tolyl | N-tolyl | b1 | b1 | b1 | b5 |
| 2-13-177 | N-tolyl | N-tolyl | b2 | b2 | b2 | b5 |
| 2-13-178 | N-tolyl | N-tolyl | b3 | b3 | b3 | b5 |
| 2-13-179 | N-tolyl | N-tolyl | H | H | H | b6 |
| 2-13-180 | N-tolyl | N-tolyl | t-bu | t-bu | t-bu | b6 |
| 2-13-181 | N-tolyl | N-tolyl | methyl | methyl | methyl | b6 |
| 2-13-182 | N-tolyl | N-tolyl | b1 | b1 | b1 | b6 |
| 2-13-183 | N-tolyl | N-tolyl | b2 | b2 | b2 | b6 |
| 2-13-184 | N-tolyl | N-tolyl | b3 | b3 | b3 | b6 |
| 2-13-185 | N-tolyl | N-tolyl | H | H | H | b7 |
| 2-13-186 | N-tolyl | N-tolyl | t-bu | t-bu | t-bu | b7 |
| 2-13-187 | N-tolyl | N-tolyl | methyl | methyl | methyl | b7 |
| 2-13-188 | N-tolyl | N-tolyl | b1 | b1 | b1 | b7 |
| 2-13-189 | N-tolyl | N-tolyl | b2 | b2 | b2 | b7 |
| 2-13-190 | N-tolyl | N-tolyl | b3 | b3 | b3 | b7 |
| 2-13-191 | N-tolyl | N-tolyl | H | H | H | b8 |
| 2-13-192 | N-tolyl | N-tolyl | t-bu | t-bu | t-bu | b8 |
| 2-13-193 | N-tolyl | N-tolyl | methyl | methyl | methyl | b8 |
| 2-13-194 | N-tolyl | N-tolyl | b1 | b1 | b1 | b8 |
| 2-13-195 | N-tolyl | N-tolyl | b2 | b2 | b2 | b8 |
| 2-13-196 | N-tolyl | N-tolyl | b3 | b3 | b3 | b8 |
| 2-13-197 | N-tolyl | N-tolyl | H | H | H | b9 |
| 2-13-198 | N-tolyl | N-tolyl | t-bu | t-bu | t-bu | b9 |
| 2-13-199 | N-tolyl | N-tolyl | methyl | methyl | methyl | b9 |
| 2-13-200 | N-tolyl | N-tolyl | b1 | b1 | b1 | b9 |
| 2-13-201 | N-tolyl | N-tolyl | b2 | b2 | b2 | b9 |
| 2-13-202 | N-tolyl | N-tolyl | b3 | b3 | b3 | b9 |
| 2-13-203 | N-tolyl | N-tolyl | H | H | H | b10 |
| 2-13-204 | N-tolyl | N-tolyl | t-bu | t-bu | t-bu | b10 |
| 2-13-205 | N-tolyl | N-tolyl | methyl | methyl | methyl | b10 |
| 2-13-206 | N-tolyl | N-tolyl | b1 | b1 | b1 | b10 |
| 2-13-207 | N-tolyl | N-tolyl | b2 | b2 | b2 | b10 |
| 2-13-208 | N-tolyl | N-tolyl | b3 | b3 | b3 | b10 |
| 2-13-209 | N-Biph | N-Biph | H | H | H | H |
| 2-13-210 | N-Biph | N-Biph | t-bu | H | t-bu | H |
| 2-13-211 | N-Biph | N-Biph | methyl | H | methyl | H |
| 2-13-212 | N-Biph | N-Biph | t-bu | t-bu | t-bu | t-bu |
| 2-13-213 | N-Biph | N-Biph | methyl | methyl | methyl | methyl |
| 2-13-214 | N-Biph | N-Biph | b1 | H | b1 | H |
| 2-13-215 | N-Biph | N-Biph | b2 | H | b2 | H |
| 2-13-216 | N-Biph | N-Biph | b3 | H | b3 | H |
| 2-13-217 | N-Biph | N-Biph | H | H | H | a1 |
| 2-13-218 | N-Biph | N-Biph | t-bu | t-bu | t-bu | a1 |
| 2-13-219 | N-Biph | N-Biph | methyl | methyl | methyl | a1 |
| 2-13-220 | N-Biph | N-Biph | b1 | b1 | b1 | a1 |
| 2-13-221 | N-Biph | N-Biph | b2 | b2 | b2 | a1 |
| 2-13-222 | N-Biph | N-Biph | b3 | b3 | b3 | a1 |
| 2-13-223 | N-Biph | N-Biph | H | H | H | a2 |
| 2-13-224 | N-Biph | N-Biph | t-bu | t-bu | t-bu | a2 |
| 2-13-225 | N-Biph | N-Biph | methyl | methyl | methyl | a2 |
| 2-13-226 | N-Biph | N-Biph | b1 | b1 | b1 | a2 |
| 2-13-227 | N-Biph | N-Biph | b2 | b2 | b2 | a2 |
| 2-13-228 | N-Biph | N-Biph | b3 | b3 | b3 | a2 |
| 2-13-229 | N-Biph | N-Biph | H | H | H | a3 |
| 2-13-230 | N-Biph | N-Biph | t-bu | t-bu | t-bu | a3 |
| 2-13-231 | N-Biph | N-Biph | methyl | methyl | methyl | a3 |
| 2-13-232 | N-Biph | N-Biph | b1 | b1 | b1 | a3 |
| 2-13-233 | N-Biph | N-Biph | b2 | b2 | b2 | a3 |
| 2-13-234 | N-Biph | N-Biph | b3 | b3 | b3 | a3 |
| 2-13-235 | N-Biph | N-Biph | H | H | H | a4 |
| 2-13-236 | N-Biph | N-Biph | t-bu | t-bu | t-bu | a4 |
| 2-13-237 | N-Biph | N-Biph | methyl | methyl | methyl | a4 |
| 2-13-238 | N-Biph | N-Biph | b1 | b1 | b1 | a4 |
| 2-13-239 | N-Biph | N-Biph | b2 | b2 | b2 | a4 |
| 2-13-240 | N-Biph | N-Biph | b3 | b3 | b3 | a4 |
| 2-13-241 | N-Biph | N-Biph | H | H | H | a5 |
| 2-13-242 | N-Biph | N-Biph | t-bu | t-bu | t-bu | a5 |
| 2-13-243 | N-Biph | N-Biph | methyl | methyl | methyl | a5 |
| 2-13-244 | N-Biph | N-Biph | b1 | b1 | b1 | a5 |
| 2-13-245 | N-Biph | N-Biph | b2 | b2 | b2 | a5 |
| 2-13-246 | N-Biph | N-Biph | b3 | b3 | b3 | a5 |
| 2-13-247 | N-Biph | N-Biph | H | H | H | a6 |
| 2-13-248 | N-Biph | N-Biph | t-bu | t-bu | t-bu | a6 |
| 2-13-249 | N-Biph | N-Biph | methyl | methyl | methyl | a6 |
| 2-13-250 | N-Biph | N-Biph | b1 | b1 | b1 | a6 |
| 2-13-251 | N-Biph | N-Biph | b2 | b2 | b2 | a6 |
| 2-13-252 | N-Biph | N-Biph | b3 | b3 | b3 | a6 |
| 2-13-253 | N-Biph | N-Biph | H | H | H | b1 |
| 2-13-254 | N-Biph | N-Biph | t-bu | t-bu | t-bu | b1 |
| 2-13-255 | N-Biph | N-Biph | methyl | methyl | methyl | b1 |
| 2-13-256 | N-Biph | N-Biph | b1 | b1 | b1 | b1 |
| 2-13-257 | N-Biph | N-Biph | b2 | b2 | b2 | b1 |
| 2-13-258 | N-Biph | N-Biph | b3 | b3 | b3 | b1 |
| 2-13-259 | N-Biph | N-Biph | H | H | H | b2 |
| 2-13-260 | N-Biph | N-Biph | t-bu | t-bu | t-bu | b2 |
| 2-13-261 | N-Biph | N-Biph | methyl | methyl | methyl | b2 |
| 2-13-262 | N-Biph | N-Biph | b1 | b1 | b1 | b2 |
| 2-13-263 | N-Biph | N-Biph | b2 | b2 | b2 | b2 |
| 2-13-264 | N-Biph | N-Biph | b3 | b3 | b3 | b2 |
| 2-13-265 | N-Biph | N-Biph | H | H | H | b3 |
| 2-13-266 | N-Biph | N-Biph | t-bu | t-bu | t-bu | b3 |
| 2-13-267 | N-Biph | N-Biph | methyl | methyl | methyl | b3 |
| 2-13-268 | N-Biph | N-Biph | b1 | b1 | b1 | b3 |
| 2-13-269 | N-Biph | N-Biph | b2 | b2 | b2 | b3 |
| 2-13-270 | N-Biph | N-Biph | b3 | b3 | b3 | b3 |
| 2-13-271 | N-Biph | N-Biph | H | H | H | b4 |
| 2-13-272 | N-Biph | N-Biph | t-bu | t-bu | t-bu | b4 |
| 2-13-273 | N-Biph | N-Biph | methyl | methyl | methyl | b4 |
| 2-13-274 | N-Biph | N-Biph | b1 | b1 | b1 | b4 |
| 2-13-275 | N-Biph | N-Biph | b2 | b2 | b2 | b4 |
| 2-13-276 | N-Biph | N-Biph | b3 | b3 | b3 | b4 |
| 2-13-277 | N-Biph | N-Biph | H | H | H | b5 |
| 2-13-278 | N-Biph | N-Biph | t-bu | t-bu | t-bu | b5 |
| 2-13-279 | N-Biph | N-Biph | methyl | methyl | methyl | b5 |
| 2-13-280 | N-Biph | N-Biph | b1 | b1 | b1 | b5 |
| 2-13-281 | N-Biph | N-Biph | b2 | b2 | b2 | b5 |
| 2-13-282 | N-Biph | N-Biph | b3 | b3 | b3 | b5 |
| 2-13-283 | N-Biph | N-Biph | H | H | H | b6 |
| 2-13-284 | N-Biph | N-Biph | t-bu | t-bu | t-bu | b6 |
| 2-13-285 | N-Biph | N-Biph | methyl | methyl | methyl | b6 |
| 2-13-286 | N-Biph | N-Biph | b1 | b1 | b1 | b6 |
| 2-13-287 | N-Biph | N-Biph | b2 | b2 | b2 | b6 |
| 2-13-288 | N-Biph | N-Biph | b3 | b3 | b3 | b6 |
| 2-13-289 | N-Biph | N-Biph | H | H | H | b7 |
| 2-13-290 | N-Biph | N-Biph | t-bu | t-bu | t-bu | b7 |
| 2-13-291 | N-Biph | N-Biph | methyl | methyl | methyl | b7 |
| 2-13-292 | N-Biph | N-Biph | b1 | b1 | b1 | b7 |
| 2-13-293 | N-Biph | N-Biph | b2 | b2 | b2 | b7 |
| 2-13-294 | N-Biph | N-Biph | b3 | b3 | b3 | b7 |
| 2-13-295 | N-Biph | N-Biph | H | H | H | b8 |
| 2-13-296 | N-Biph | N-Biph | t-bu | t-bu | t-bu | b8 |
| 2-13-297 | N-Biph | N-Biph | methyl | methyl | methyl | b8 |
| 2-13-298 | N-Biph | N-Biph | b1 | b1 | b1 | b8 |
| 2-13-299 | N-Biph | N-Biph | b2 | b2 | b2 | b8 |
| 2-13-300 | N-Biph | N-Biph | b3 | b3 | b3 | b8 |
| 2-13-301 | N-Biph | N-Biph | H | H | H | b9 |
| 2-13-302 | N-Biph | N-Biph | t-bu | t-bu | t-bu | b9 |
| 2-13-303 | N-Biph | N-Biph | methyl | methyl | methyl | b9 |
| 2-13-304 | N-Biph | N-Biph | b1 | b1 | b1 | b9 |
| 2-13-305 | N-Biph | N-Biph | b2 | b2 | b2 | b9 |
| 2-13-306 | N-Biph | N-Biph | b3 | b3 | b3 | b9 |
| 2-13-307 | N-Biph | N-Biph | H | H | H | b10 |
| 2-13-308 | N-Biph | N-Biph | t-bu | t-bu | t-bu | b10 |
| 2-13-309 | N-Biph | N-Biph | methyl | methyl | methyl | b10 |
| 2-13-310 | N-Biph | N-Biph | b1 | b1 | b1 | b10 |
| 2-13-311 | N-Biph | N-Biph | b2 | b2 | b2 | b10 |
| 2-13-312 | N-Biph | N-Biph | b3 | b3 | b3 | b10 |
| 2-13-313 | O | N—Ph | H | H | H | H |
| 2-13-314 | O | N—Ph | t-bu | H | t-bu | H |
| 2-13-315 | O | N—Ph | methyl | H | methyl | H |
| 2-13-316 | O | N—Ph | t-bu | t-bu | t-bu | t-bu |
| 2-13-317 | O | N—Ph | methyl | methyl | methyl | methyl |
| 2-13-318 | O | N—Ph | b1 | H | b1 | H |
| 2-13-319 | O | N—Ph | b2 | H | b2 | H |
| 2-13-320 | O | N—Ph | b3 | H | b3 | H |
| 2-13-321 | O | N—Ph | H | H | H | a1 |
| 2-13-322 | O | N—Ph | t-bu | t-bu | t-bu | a1 |
| 2-13-323 | O | N—Ph | methyl | methyl | methyl | a1 |
| 2-13-324 | O | N—Ph | b1 | b1 | b1 | a1 |
| 2-13-325 | O | N—Ph | b2 | b2 | b2 | a1 |
| 2-13-326 | O | N—Ph | b3 | b3 | b3 | a1 |
| 2-13-327 | O | N—Ph | H | H | H | a2 |
| 2-13-328 | O | N—Ph | t-bu | t-bu | t-bu | a2 |
| 2-13-329 | O | N—Ph | methyl | methyl | methyl | a2 |
| 2-13-330 | O | N—Ph | b1 | b1 | b1 | a2 |
| 2-13-331 | O | N—Ph | b2 | b2 | b2 | a2 |
| 2-13-332 | O | N—Ph | b3 | b3 | b3 | a2 |
| 2-13-333 | O | N—Ph | H | H | H | a3 |
| 2-13-334 | O | N—Ph | t-bu | t-bu | t-bu | a3 |
| 2-13-335 | O | N—Ph | methyl | methyl | methyl | a3 |
| 2-13-336 | O | N—Ph | b1 | b1 | b1 | a3 |
| 2-13-337 | O | N—Ph | b2 | b2 | b2 | a3 |
| 2-13-338 | O | N—Ph | b3 | b3 | b3 | a3 |
| 2-13-339 | O | N—Ph | H | H | H | a4 |
| 2-13-340 | O | N—Ph | t-bu | t-bu | t-bu | a4 |
| 2-13-341 | O | N—Ph | methyl | methyl | methyl | a4 |
| 2-13-342 | O | N—Ph | b1 | b1 | b1 | a4 |
| 2-13-343 | O | N—Ph | b2 | b2 | b2 | a4 |
| 2-13-344 | O | N—Ph | b3 | b3 | b3 | a4 |
| 2-13-345 | O | N—Ph | H | H | H | a5 |
| 2-13-346 | O | N—Ph | t-bu | t-bu | t-bu | a5 |
| 2-13-347 | O | N—Ph | methyl | methyl | methyl | a5 |
| 2-13-348 | O | N—Ph | b1 | b1 | b1 | a5 |
| 2-13-349 | O | N—Ph | b2 | b2 | b2 | a5 |
| 2-13-350 | O | N—Ph | b3 | b3 | b3 | a5 |
| 2-13-351 | O | N—Ph | H | H | H | a6 |
| 2-13-352 | O | N—Ph | t-bu | t-bu | t-bu | a6 |
| 2-13-353 | O | N—Ph | methyl | methyl | methyl | a6 |
| 2-13-354 | O | N—Ph | b1 | b1 | b1 | a6 |
| 2-13-355 | O | N—Ph | b2 | b2 | b2 | a6 |
| 2-13-356 | O | N—Ph | b3 | b3 | b3 | a6 |
| 2-13-357 | O | N—Ph | H | H | H | b1 |
| 2-13-358 | O | N—Ph | t-bu | t-bu | t-bu | b1 |
| 2-13-359 | O | N—Ph | methyl | methyl | methyl | b1 |
| 2-13-360 | O | N—Ph | b1 | b1 | b1 | b1 |
| 2-13-361 | O | N—Ph | b2 | b2 | b2 | b1 |
| 2-13-362 | O | N—Ph | b3 | b3 | b3 | b1 |
| 2-13-363 | O | N—Ph | H | H | H | b2 |
| 2-13-364 | O | N—Ph | t-bu | t-bu | t-bu | b2 |
| 2-13-365 | O | N—Ph | methyl | methyl | methyl | b2 |
| 2-13-366 | O | N—Ph | b1 | b1 | b1 | b2 |
| 2-13-367 | O | N—Ph | b2 | b2 | b2 | b2 |
| 2-13-368 | O | N—Ph | b3 | b3 | b3 | b2 |
| 2-13-369 | O | N—Ph | H | H | H | b3 |
| 2-13-370 | O | N—Ph | t-bu | t-bu | t-bu | b3 |
| 2-13-371 | O | N—Ph | methyl | methyl | methyl | b3 |
| 2-13-372 | O | N—Ph | b1 | b1 | b1 | b3 |
| 2-13-373 | O | N—Ph | b2 | b2 | b2 | b3 |
| 2-13-374 | O | N—Ph | b3 | b3 | b3 | b3 |
| 2-13-375 | O | N—Ph | H | H | H | b4 |
| 2-13-376 | O | N—Ph | t-bu | t-bu | t-bu | b4 |
| 2-13-377 | O | N—Ph | methyl | methyl | methyl | b4 |
| 2-13-378 | O | N—Ph | b1 | b1 | b1 | b4 |
| 2-13-379 | O | N—Ph | b2 | b2 | b2 | b4 |
| 2-13-380 | O | N—Ph | b3 | b3 | b3 | b4 |
| 2-13-381 | O | N—Ph | H | H | H | b5 |
| 2-13-382 | O | N—Ph | t-bu | t-bu | t-bu | b5 |
| 2-13-383 | O | N—Ph | methyl | methyl | methyl | b5 |
| 2-13-384 | O | N—Ph | b1 | b1 | b1 | b5 |
| 2-13-385 | O | N—Ph | b2 | b2 | b2 | b5 |
| 2-13-386 | O | N—Ph | b3 | b3 | b3 | b5 |
| 2-13-387 | O | N—Ph | H | H | H | b6 |
| 2-13-388 | O | N—Ph | t-bu | t-bu | t-bu | b6 |
| 2-13-389 | O | N—Ph | methyl | methyl | methyl | b6 |
| 2-13-390 | O | N—Ph | b1 | b1 | b1 | b6 |
| 2-13-391 | O | N—Ph | b2 | b2 | b2 | b6 |
| 2-13-392 | O | N—Ph | b3 | b3 | b3 | b6 |
| 2-13-393 | O | N—Ph | H | H | H | b7 |
| 2-13-394 | O | N—Ph | t-bu | t-bu | t-bu | b7 |
| 2-13-395 | O | N—Ph | methyl | methyl | methyl | b7 |
| 2-13-396 | O | N—Ph | b1 | b1 | b1 | b7 |
| 2-13-397 | O | N—Ph | b2 | b2 | b2 | b7 |
| 2-13-398 | O | N—Ph | b3 | b3 | b3 | b7 |
| 2-13-399 | O | N—Ph | H | H | H | b8 |
| 2-13-400 | O | N—Ph | t-bu | t-bu | t-bu | b8 |
| 2-13-401 | O | N—Ph | methyl | methyl | methyl | b8 |
| 2-13-402 | O | N—Ph | b1 | b1 | b1 | b8 |
| 2-13-403 | O | N—Ph | b2 | b2 | b2 | b8 |
| 2-13-404 | O | N—Ph | b3 | b3 | b3 | b8 |
| 2-13-405 | O | N—Ph | H | H | H | b9 |
| 2-13-406 | O | N—Ph | t-bu | t-bu | t-bu | b9 |
| 2-13-407 | O | N—Ph | methyl | methyl | methyl | b9 |
| 2-13-408 | O | N—Ph | b1 | b1 | b1 | b9 |
| 2-13-409 | O | N—Ph | b2 | b2 | b2 | b9 |
| 2-13-410 | O | N—Ph | b3 | b3 | b3 | b9 |
| 2-13-411 | O | N—Ph | H | H | H | b10 |
| 2-13-412 | O | N—Ph | t-bu | t-bu | t-bu | b10 |
| 2-13-413 | O | N—Ph | methyl | methyl | methyl | b10 |
| 2-13-414 | O | N—Ph | b1 | b1 | b1 | b10 |
| 2-13-415 | O | N—Ph | b2 | b2 | b2 | b10 |
| 2-13-416 | O | N—Ph | b3 | b3 | b3 | b10 |
| 2-13-417 | S | N—Ph | H | H | H | H |
| 2-13-418 | S | N—Ph | t-bu | H | t-bu | H |
| 2-13-419 | S | N—Ph | methyl | H | methyl | H |
| 2-13-420 | S | N—Ph | t-bu | t-bu | t-bu | t-bu |
| 2-13-421 | S | N—Ph | methyl | methyl | methyl | methyl |
| 2-13-422 | S | N—Ph | b1 | H | b1 | H |
| 2-13-423 | S | N—Ph | b2 | H | b2 | H |
| 2-13-424 | S | N—Ph | b3 | H | b3 | H |
| 2-13-425 | S | N—Ph | H | H | H | a1 |
| 2-13-426 | S | N—Ph | t-bu | t-bu | t-bu | a1 |
| 2-13-427 | S | N—Ph | methyl | methyl | methyl | a1 |
| 2-13-428 | S | N—Ph | b1 | b1 | b1 | a1 |
| 2-13-429 | S | N—Ph | b2 | b2 | b2 | a1 |
| 2-13-430 | S | N—Ph | b3 | b3 | b3 | a1 |
| 2-13-431 | S | N—Ph | H | H | H | a2 |
| 2-13-432 | S | N—Ph | t-bu | t-bu | t-bu | a2 |
| 2-13-433 | S | N—Ph | methyl | methyl | methyl | a2 |
| 2-13-434 | S | N—Ph | b1 | b1 | b1 | a2 |
| 2-13-435 | S | N—Ph | b2 | b2 | b2 | a2 |
| 2-13-436 | S | N—Ph | b3 | b3 | b3 | a2 |
| 2-13-437 | S | N—Ph | H | H | H | a3 |
| 2-13-438 | S | N—Ph | t-bu | t-bu | t-bu | a3 |
| 2-13-439 | S | N—Ph | methyl | methyl | methyl | a3 |
| 2-13-440 | S | N—Ph | b1 | b1 | b1 | a3 |
| 2-13-441 | S | N—Ph | b2 | b2 | b2 | a3 |
| 2-13-442 | S | N—Ph | b3 | b3 | b3 | a3 |
| 2-13-443 | S | N—Ph | H | H | H | a4 |
| 2-13-444 | S | N—Ph | t-bu | t-bu | t-bu | a4 |
| 2-13-445 | S | N—Ph | methyl | methyl | methyl | a4 |
| 2-13-446 | S | N—Ph | b1 | b1 | b1 | a4 |
| 2-13-447 | S | N—Ph | b2 | b2 | b2 | a4 |
| 2-13-448 | S | N—Ph | b3 | b3 | b3 | a4 |
| 2-13-449 | S | N—Ph | H | H | H | a5 |
| 2-13-450 | S | N—Ph | t-bu | t-bu | t-bu | a5 |
| 2-13-451 | S | N—Ph | methyl | methyl | methyl | a5 |
| 2-13-452 | S | N—Ph | b1 | b1 | b1 | a5 |
| 2-13-453 | S | N—Ph | b2 | b2 | b2 | a5 |
| 2-13-454 | S | N—Ph | b3 | b3 | b3 | a5 |
| 2-13-455 | S | N—Ph | H | H | H | a6 |
| 2-13-456 | S | N—Ph | t-bu | t-bu | t-bu | a6 |
| 2-13-457 | S | N—Ph | methyl | methyl | methyl | a6 |
| 2-13-458 | S | N—Ph | b1 | b1 | b1 | a6 |
| 2-13-459 | S | N—Ph | b2 | b2 | b2 | a6 |
| 2-13-460 | S | N—Ph | b3 | b3 | b3 | a6 |
| 2-13-461 | S | N—Ph | H | H | H | b1 |
| 2-13-462 | S | N—Ph | t-bu | t-bu | t-bu | b1 |
| 2-13-463 | S | N—Ph | methyl | methyl | methyl | b1 |
| 2-13-464 | S | N—Ph | b1 | b1 | b1 | b1 |
| 2-13-465 | S | N—Ph | b2 | b2 | b2 | b1 |
| 2-13-466 | S | N—Ph | b3 | b3 | b3 | b1 |
| 2-13-467 | S | N—Ph | H | H | H | b2 |
| 2-13-468 | S | N—Ph | t-bu | t-bu | t-bu | b2 |
| 2-13-469 | S | N—Ph | methyl | methyl | methyl | b2 |
| 2-13-470 | S | N—Ph | b1 | b1 | b1 | b2 |
| 2-13-471 | S | N—Ph | b2 | b2 | b2 | b2 |
| 2-13-472 | S | N—Ph | b3 | b3 | b3 | b2 |
| 2-13-473 | S | N—Ph | H | H | H | b3 |
| 2-13-474 | S | N—Ph | t-bu | t-bu | t-bu | b3 |
| 2-13-475 | S | N—Ph | methyl | methyl | methyl | b3 |
| 2-13-476 | S | N—Ph | b1 | b1 | b1 | b3 |
| 2-13-477 | S | N—Ph | b2 | b2 | b2 | b3 |
| 2-13-478 | S | N—Ph | b3 | b3 | b3 | b3 |
| 2-13-479 | S | N—Ph | H | H | H | b4 |
| 2-13-480 | S | N—Ph | t-bu | t-bu | t-bu | b4 |
| 2-13-481 | S | N—Ph | methyl | methyl | methyl | b4 |
| 2-13-482 | S | N—Ph | b1 | b1 | b1 | b4 |
| 2-13-483 | S | N—Ph | b2 | b2 | b2 | b4 |
| 2-13-484 | S | N—Ph | b3 | b3 | b3 | b4 |
| 2-13-485 | S | N—Ph | H | H | H | b5 |
| 2-13-486 | S | N—Ph | t-bu | t-bu | t-bu | b5 |
| 2-13-487 | S | N—Ph | methyl | methyl | methyl | b5 |
| 2-13-488 | S | N—Ph | b1 | b1 | b1 | b5 |
| 2-13-489 | S | N—Ph | b2 | b2 | b2 | b5 |
| 2-13-490 | S | N—Ph | b3 | b3 | b3 | b5 |
| 2-13-491 | S | N—Ph | H | H | H | b6 |
| 2-13-492 | S | N—Ph | t-bu | t-bu | t-bu | b6 |
| 2-13-493 | S | N—Ph | methyl | methyl | methyl | b6 |
| 2-13-494 | S | N—Ph | b1 | b1 | b1 | b6 |
| 2-13-495 | S | N—Ph | b2 | b2 | b2 | b6 |
| 2-13-496 | S | N—Ph | b3 | b3 | b3 | b6 |
| 2-13-497 | S | N—Ph | H | H | H | b7 |
| 2-13-498 | S | N—Ph | t-bu | t-bu | t-bu | b7 |
| 2-13-499 | S | N—Ph | methyl | methyl | methyl | b7 |
| 2-13-500 | S | N—Ph | b1 | b1 | b1 | b7 |
| 2-13-501 | S | N—Ph | b2 | b2 | b2 | b7 |
| 2-13-502 | S | N—Ph | b3 | b3 | b3 | b7 |
| 2-13-503 | S | N—Ph | H | H | H | b8 |
| 2-13-504 | S | N—Ph | t-bu | t-bu | t-bu | b8 |
| 2-13-505 | S | N—Ph | methyl | methyl | methyl | b8 |
| 2-13-506 | S | N—Ph | b1 | b1 | b1 | b8 |
| 2-13-507 | S | N—Ph | b2 | b2 | b2 | b8 |
| 2-13-508 | S | N—Ph | b3 | b3 | b3 | b8 |
| 2-13-509 | S | N—Ph | H | H | H | b9 |
| 2-13-510 | S | N—Ph | t-bu | t-bu | t-bu | b9 |
| 2-13-511 | S | N—Ph | methyl | methyl | methyl | b9 |
| 2-13-512 | S | N—Ph | b1 | b1 | b1 | b9 |
| 2-13-513 | S | N—Ph | b2 | b2 | b2 | b9 |
| 2-13-514 | S | N—Ph | b3 | b3 | b3 | b9 |
| 2-13-515 | S | N—Ph | H | H | H | b10 |
| 2-13-516 | S | N—Ph | t-bu | t-bu | t-bu | b10 |
| 2-13-517 | S | N—Ph | methyl | methyl | methyl | b10 |
| 2-13-518 | S | N—Ph | b1 | b1 | b1 | b10 |
| 2-13-519 | S | N—Ph | b2 | b2 | b2 | b10 |
| 2-13-520 | S | N—Ph | b3 | b3 | b3 | b10 |

### Group 2-14

The compounds are represented by the formula 2-14 below, wherein X₁ is B, and Y1, Y2, R1b, R1d, R2b, R2d, R3a are each shown in the table 14 below,

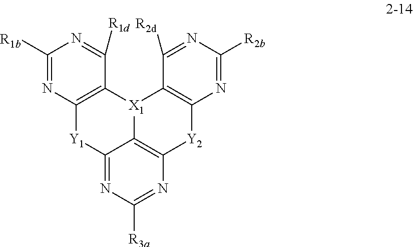

TABLE 14

| Compound No. | Y1 | Y2 | R1b | R1d | R2b | R2d | R3a |
|---|---|---|---|---|---|---|---|
| 2-14-001 | N—Ph | N—Ph | H | H | H | H | H |
| 2-14-002 | N—Ph | N—Ph | t-bu | H | t-bu | H | H |
| 2-14-003 | N—Ph | N—Ph | methyl | H | methyl | H | H |
| 2-14-004 | N—Ph | N—Ph | t-bu | t-bu | t-bu | t-bu | t-bu |
| 2-14-005 | N—Ph | N—Ph | methyl | methyl | methyl | methyl | methyl |
| 2-14-006 | N—Ph | N—Ph | b1 | H | b1 | H | H |
| 2-14-007 | N—Ph | N—Ph | b2 | H | b2 | H | H |
| 2-14-008 | N—Ph | N—Ph | b3 | H | b3 | H | H |
| 2-14-009 | N—Ph | N—Ph | t-bu | H | t-bu | H | t-bu |
| 2-14-010 | N—Ph | N—Ph | methyl | H | methyl | H | methyl |
| 2-14-011 | N—Ph | N—Ph | b1 | H | b1 | H | b1 |
| 2-14-012 | N—Ph | N—Ph | b2 | H | b2 | H | b2 |
| 2-14-013 | N—Ph | N—Ph | b3 | H | b3 | H | b3 |
| 2-14-014 | N-tolyl | N-tolyl | H | H | H | H | H |
| 2-14-015 | N-tolyl | N-tolyl | t-bu | H | t-bu | H | H |
| 2-14-016 | N-tolyl | N-tolyl | methyl | H | methyl | H | H |
| 2-14-017 | N-tolyl | N-tolyl | t-bu | t-bu | t-bu | t-bu | t-bu |
| 2-14-018 | N-tolyl | N-tolyl | methyl | methyl | methyl | methyl | methyl |
| 2-14-019 | N-tolyl | N-tolyl | b1 | H | b1 | H | H |
| 2-14-020 | N-tolyl | N-tolyl | b2 | H | b2 | H | H |
| 2-14-021 | N-tolyl | N-tolyl | b3 | H | b3 | H | H |
| 2-14-022 | N-tolyl | N-tolyl | t-bu | H | t-bu | H | t-bu |
| 2-14-023 | N-tolyl | N-tolyl | methyl | H | methyl | H | methyl |
| 2-14-024 | N-tolyl | N-tolyl | b1 | H | b1 | H | b1 |
| 2-14-025 | N-tolyl | N-tolyl | b2 | H | b2 | H | b2 |
| 2-14-026 | N-tolyl | N-tolyl | b3 | H | b3 | H | b3 |
| 2-14-027 | N-Biph | N-Biph | H | H | H | H | H |
| 2-14-028 | N-Biph | N-Biph | t-bu | H | t-bu | H | H |
| 2-14-029 | N-Biph | N-Biph | methyl | H | methyl | H | H |
| 2-14-030 | N-Biph | N-Biph | t-bu | t-bu | t-bu | t-bu | t-bu |
| 2-14-031 | N-Biph | N-Biph | methyl | methyl | methyl | methyl | methyl |
| 2-14-032 | N-Biph | N-Biph | b1 | H | b1 | H | H |
| 2-14-033 | N-Biph | N-Biph | b2 | H | b2 | H | H |
| 2-14-034 | N-Biph | N-Biph | b3 | H | b3 | H | H |
| 2-14-035 | N-Biph | N-Biph | t-bu | H | t-bu | H | t-bu |
| 2-14-036 | N-Biph | N-Biph | methyl | H | methyl | H | methyl |
| 2-14-037 | N-Biph | N-Biph | b1 | H | b1 | H | b1 |
| 2-14-038 | N-Biph | N-Biph | b2 | H | b2 | H | b2 |
| 2-14-039 | N-Biph | N-Biph | b3 | H | b3 | H | b3 |
| 2-14-040 | N—Ph | O | H | H | H | H | H |
| 2-14-041 | N—Ph | O | t-bu | H | t-bu | H | H |
| 2-14-042 | N—Ph | O | methyl | H | methyl | H | H |
| 2-14-043 | N—Ph | O | t-bu | t-bu | t-bu | t-bu | t-bu |
| 2-14-044 | N—Ph | O | methyl | methyl | methyl | methyl | methyl |
| 2-14-045 | N—Ph | O | b1 | H | b1 | H | H |
| 2-14-046 | N—Ph | O | b2 | H | b2 | H | H |
| 2-14-047 | N—Ph | O | b3 | H | b3 | H | H |
| 2-14-048 | N—Ph | O | t-bu | H | t-bu | H | t-bu |
| 2-14-049 | N—Ph | O | methyl | H | methyl | H | methyl |
| 2-14-050 | N—Ph | O | b1 | H | b1 | H | b1 |
| 2-14-051 | N—Ph | O | b2 | H | b2 | H | b2 |
| 2-14-052 | N—Ph | O | b3 | H | b3 | H | b3 |
| 2-14-053 | N—Ph | S | H | H | H | H | H |
| 2-14-054 | N—Ph | S | t-bu | H | t-bu | H | H |
| 2-14-055 | N—Ph | S | methyl | H | methyl | H | H |
| 2-14-056 | N—Ph | S | t-bu | t-bu | t-bu | t-bu | t-bu |
| 2-14-057 | N—Ph | S | methyl | methyl | methyl | methyl | methyl |
| 2-14-058 | N—Ph | S | b1 | H | b1 | H | H |
| 2-14-059 | N—Ph | S | b2 | H | b2 | H | H |
| 2-14-060 | N—Ph | S | b3 | H | b3 | H | H |
| 2-14-061 | N—Ph | S | t-bu | H | t-bu | H | t-bu |
| 2-14-062 | N—Ph | S | methyl | H | methyl | H | methyl |
| 2-14-063 | N—Ph | S | b1 | H | b1 | H | b1 |
| 2-14-064 | N—Ph | S | b2 | H | b2 | H | b2 |
| 2-14-065 | N—Ph | S | b3 | H | b3 | H | b3 |

### Group 2-15

The compounds are represented by the formula 2-15 below, wherein X₁ is B, and Y1, Y2, R1b, R1d, R2a, R2c, R3a are each shown in the table 15 below,

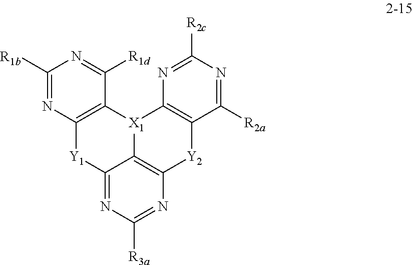

TABLE 15

| Compound No. | Y1 | Y2 | R1b | R1d | R2a | R2c | R3a |
|---|---|---|---|---|---|---|---|
| 2-15-001 | N—Ph | N—Ph | H | H | H | H | H |
| 2-15-002 | N—Ph | N—Ph | t-bu | H | t-bu | H | H |
| 2-15-003 | N—Ph | N—Ph | methyl | H | methyl | H | H |
| 2-15-004 | N—Ph | N—Ph | t-bu | t-bu | t-bu | t-bu | t-bu |
| 2-15-005 | N—Ph | N—Ph | methyl | methyl | methyl | methyl | methyl |
| 2-15-006 | N—Ph | N—Ph | b1 | H | b1 | b1 | H |
| 2-15-007 | N—Ph | N—Ph | b2 | H | b2 | b2 | H |
| 2-15-008 | N—Ph | N—Ph | b3 | H | b3 | b3 | H |
| 2-15-009 | N—Ph | N—Ph | b1 | H | b1 | b1 | b1 |
| 2-15-010 | N—Ph | N—Ph | b2 | H | b2 | b2 | b2 |
| 2-15-011 | N—Ph | N—Ph | b3 | H | b3 | b3 | b3 |
| 2-15-012 | N-tolyl | N-tolyl | H | H | H | H | H |
| 2-15-013 | N-tolyl | N-tolyl | t-bu | H | t-bu | H | H |
| 2-15-014 | N-tolyl | N-tolyl | methyl | H | methyl | H | H |
| 2-15-015 | N-tolyl | N-tolyl | t-bu | t-bu | t-bu | t-bu | t-bu |
| 2-15-016 | N-tolyl | N-tolyl | methyl | methyl | methyl | methyl | methyl |
| 2-15-017 | N-tolyl | N-tolyl | b1 | H | b1 | b1 | H |
| 2-15-018 | N-tolyl | N-tolyl | b2 | H | b2 | b2 | H |
| 2-15-019 | N-tolyl | N-tolyl | b3 | H | b3 | b3 | H |
| 2-15-020 | N-tolyl | N-tolyl | b1 | H | b1 | b1 | b1 |
| 2-15-021 | N-tolyl | N-tolyl | b2 | H | b2 | b2 | b2 |
| 2-15-022 | N-tolyl | N-tolyl | b3 | H | b3 | b3 | b3 |
| 2-15-023 | N-Biph | N-Biph | H | H | H | H | H |
| 2-15-024 | N-Biph | N-Biph | t-bu | H | t-bu | H | H |
| 2-15-025 | N-Biph | N-Biph | methyl | H | methyl | H | H |
| 2-15-026 | N-Biph | N-Biph | t-bu | t-bu | t-bu | t-bu | t-bu |
| 2-15-027 | N-Biph | N-Biph | methyl | methyl | methyl | methyl | methyl |
| 2-15-028 | N-Biph | N-Biph | b1 | H | b1 | b1 | H |
| 2-15-029 | N-Biph | N-Biph | b2 | H | b2 | b2 | H |
| 2-15-030 | N-Biph | N-Biph | b3 | H | b3 | b3 | H |
| 2-15-031 | N-Biph | N-Biph | b1 | H | b1 | b1 | b1 |
| 2-15-032 | N-Biph | N-Biph | b2 | H | b2 | b2 | b2 |
| 2-15-033 | N-Biph | N-Biph | b3 | H | b3 | b3 | b3 |
| 2-15-034 | N—Ph | O | H | H | H | H | H |
| 2-15-035 | N—Ph | O | t-bu | H | t-bu | H | H |
| 2-15-036 | N—Ph | O | methyl | H | methyl | H | H |
| 2-15-037 | N—Ph | O | t-bu | t-bu | t-bu | t-bu | t-bu |
| 2-15-038 | N—Ph | O | methyl | methyl | methyl | methyl | methyl |
| 2-15-039 | N—Ph | O | b1 | H | b1 | b1 | H |
| 2-15-040 | N—Ph | O | b2 | H | b2 | b2 | H |
| 2-15-041 | N—Ph | O | b3 | H | b3 | b3 | H |
| 2-15-042 | N—Ph | O | b1 | H | b1 | b1 | b1 |
| 2-15-043 | N—Ph | O | b2 | H | b2 | b2 | b2 |
| 2-15-044 | N—Ph | O | b3 | H | b3 | b3 | b3 |
| 2-15-045 | N—Ph | S | H | H | H | H | H |
| 2-15-046 | N—Ph | S | t-bu | H | t-bu | H | H |
| 2-15-047 | N—Ph | S | methyl | H | methyl | H | H |
| 2-15-048 | N—Ph | S | t-bu | t-bu | t-bu | t-bu | t-bu |
| 2-15-049 | N—Ph | S | methyl | methyl | methyl | methyl | Methyl |
| 2-15-050 | N—Ph | S | b1 | H | b1 | b1 | H |
| 2-15-051 | N—Ph | S | b2 | H | b2 | b2 | H |
| 2-15-052 | N—Ph | S | b3 | H | b3 | b3 | H |
| 2-15-053 | N—Ph | S | b1 | H | b1 | b1 | b1 |
| 2-15-054 | N—Ph | S | b2 | H | b2 | b2 | b2 |
| 2-15-055 | N—Ph | S | b3 | H | b3 | b3 | b3 |

### Group 2-16

The compounds are represented by the formula 2-16 below, wherein X₁ is B, and Y1, Y2, R1a, R1c, R2b, R2d, R3a are each shown in the table 16 below,

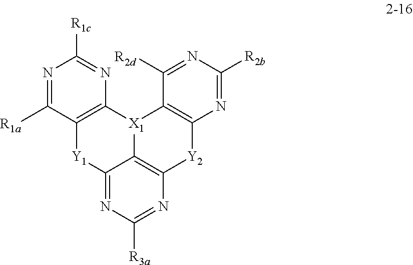

TABLE 16

| Compound No. | Y1 | Y2 | R1a | R1c | R2b | R2d | R3a |
|---|---|---|---|---|---|---|---|
| 2-16-001 | N—Ph | N—Ph | H | H | H | H | H |
| 2-16-002 | N—Ph | N—Ph | t-bu | H | t-bu | H | H |
| 2-16-003 | N—Ph | N—Ph | methyl | H | methyl | H | H |
| 2-16-004 | N—Ph | N—Ph | t-bu | t-bu | t-bu | t-bu | t-bu |
| 2-16-005 | N—Ph | N—Ph | methyl | methyl | methyl | methyl | methyl |
| 2-16-006 | N—Ph | N—Ph | b1 | b1 | b1 | H | H |
| 2-16-007 | N—Ph | N—Ph | b2 | b2 | b2 | H | H |
| 2-16-008 | N—Ph | N—Ph | b3 | b3 | b3 | H | H |
| 2-16-009 | N—Ph | N—Ph | b1 | b1 | b1 | H | b1 |
| 2-16-010 | N—Ph | N—Ph | b2 | b2 | b2 | H | b2 |
| 2-16-011 | N—Ph | N—Ph | b3 | b3 | b3 | H | b3 |
| 2-16-012 | N-tolyl | N-tolyl | H | H | H | H | H |
| 2-16-013 | N-tolyl | N-tolyl | t-bu | H | t-bu | H | H |
| 2-16-014 | N-tolyl | N-tolyl | methyl | H | methyl | H | H |
| 2-16-015 | N-tolyl | N-tolyl | t-bu | t-bu | t-bu | t-bu | t-bu |
| 2-16-016 | N-tolyl | N-tolyl | methyl | methyl | methyl | methyl | methyl |
| 2-16-017 | N-tolyl | N-tolyl | b1 | b1 | b1 | H | H |
| 2-16-018 | N-tolyl | N-tolyl | b2 | b2 | b2 | H | H |
| 2-16-019 | N-tolyl | N-tolyl | b3 | b3 | b3 | H | H |
| 2-16-020 | N-tolyl | N-tolyl | b1 | b1 | b1 | H | b1 |
| 2-16-021 | N-tolyl | N-tolyl | b2 | b2 | b2 | H | b2 |
| 2-16-022 | N-tolyl | N-tolyl | b3 | b3 | b3 | H | b3 |
| 2-16-023 | N-Biph | N-Biph | H | H | H | H | H |
| 2-16-024 | N-Biph | N-Biph | t-bu | H | t-bu | H | H |
| 2-16-025 | N-Biph | N-Biph | methyl | H | methyl | H | H |
| 2-16-026 | N-Biph | N-Biph | t-bu | t-bu | t-bu | t-bu | t-bu |
| 2-16-027 | N-Biph | N-Biph | methyl | methyl | methyl | methyl | methyl |
| 2-16-028 | N-Biph | N-Biph | b1 | b1 | b1 | H | H |
| 2-16-029 | N-Biph | N-Biph | b2 | b2 | b2 | H | H |
| 2-16-030 | N-Biph | N-Biph | b3 | b3 | b3 | H | H |
| 2-16-031 | N-Biph | N-Biph | b1 | b1 | b1 | H | b1 |
| 2-16-032 | N-Biph | N-Biph | b2 | b2 | b2 | H | b2 |
| 2-16-033 | N-Biph | N-Biph | b3 | b3 | b3 | H | b3 |
| 2-16-034 | N—Ph | O | H | H | H | H | H |
| 2-16-035 | N—Ph | O | t-bu | H | t-bu | H | H |
| 2-16-036 | N—Ph | O | methyl | H | methyl | H | H |
| 2-16-037 | N—Ph | O | t-bu | t-bu | t-bu | t-bu | t-bu |
| 2-16-038 | N—Ph | O | methyl | methyl | methyl | methyl | methyl |
| 2-16-039 | N—Ph | O | b1 | b1 | b1 | H | H |
| 2-16-040 | N—Ph | O | b2 | b2 | b2 | H | H |
| 2-16-041 | N—Ph | O | b3 | b3 | b3 | H | H |
| 2-16-042 | N—Ph | O | b1 | b1 | b1 | H | b1 |
| 2-16-043 | N—Ph | O | b2 | b2 | b2 | H | b2 |
| 2-16-044 | N—Ph | O | b3 | b3 | b3 | H | b3 |
| 2-16-045 | N—Ph | S | H | H | H | H | H |
| 2-16-046 | N—Ph | S | t-bu | H | t-bu | H | H |
| 2-16-047 | N—Ph | S | methyl | H | methyl | H | H |
| 2-16-048 | N—Ph | S | t-bu | t-bu | t-bu | t-bu | t-bu |
| 2-16-049 | N—Ph | S | methyl | methyl | methyl | methyl | methyl |
| 2-16-050 | N—Ph | S | b1 | b1 | b1 | H | H |
| 2-16-051 | N—Ph | S | b2 | b2 | b2 | H | H |
| 2-16-052 | N—Ph | S | b3 | b3 | b3 | H | H |
| 2-16-053 | N—Ph | S | b1 | b1 | b1 | H | b1 |
| 2-16-054 | N—Ph | S | b2 | b2 | b2 | H | b2 |
| 2-16-055 | N—Ph | S | b3 | b3 | b3 | H | b3 |

### Group 2-17

The compounds are represented by the formula 2-17 below, wherein X₁ is B, and Y1, Y2, R1a, R1c, R2a, R2c, R3a are each shown in the table 17 below,

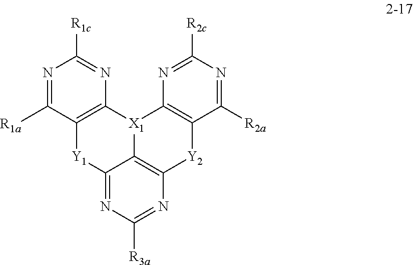

TABLE 17

| Compound No. | Y1 | Y2 | R1a | R1c | R2a | R2c | R3a |
|---|---|---|---|---|---|---|---|
| 2-17-001 | N—Ph | N—Ph | H | H | H | H | H |
| 2-17-002 | N—Ph | N—Ph | t-bu | H | t-bu | H | H |
| 2-17-003 | N—Ph | N—Ph | methyl | H | methyl | H | H |
| 2-17-004 | N—Ph | N—Ph | t-bu | t-bu | t-bu | t-bu | t-bu |
| 2-17-005 | N—Ph | N—Ph | methyl | methyl | methyl | methyl | methyl |
| 2-17-006 | N—Ph | N—Ph | b1 | H | b1 | H | H |
| 2-17-007 | N—Ph | N—Ph | b2 | H | b2 | H | H |
| 2-17-008 | N—Ph | N—Ph | b3 | H | b3 | H | H |
| 2-17-009 | N—Ph | N—Ph | t-bu | H | t-bu | H | t-bu |
| 2-17-010 | N—Ph | N—Ph | methyl | H | methyl | H | methyl |
| 2-17-011 | N—Ph | N—Ph | b1 | H | b1 | H | b1 |
| 2-17-012 | N—Ph | N—Ph | b2 | H | b2 | H | b2 |
| 2-17-013 | N—Ph | N—Ph | b3 | H | b3 | H | b3 |
| 2-17-014 | N—Ph | N—Ph | b1 | b1 | b1 | b1 | H |
| 2-17-015 | N—Ph | N—Ph | b2 | b2 | b2 | b2 | H |
| 2-17-016 | N—Ph | N—Ph | b3 | b3 | b3 | b3 | H |
| 2-17-017 | N—Ph | N—Ph | b1 | b1 | b1 | b1 | b1 |
| 2-17-018 | N—Ph | N—Ph | b2 | b2 | b2 | b2 | b2 |
| 2-17-019 | N—Ph | N—Ph | b3 | b3 | b3 | b3 | b3 |
| 2-17-020 | N-tolyl | N-tolyl | H | H | H | H | H |
| 2-17-021 | N-tolyl | N-tolyl | t-bu | H | t-bu | H | H |
| 2-17-022 | N-tolyl | N-tolyl | methyl | H | methyl | H | H |
| 2-17-023 | N-tolyl | N-tolyl | t-bu | t-bu | t-bu | t-bu | t-bu |
| 2-17-024 | N-tolyl | N-tolyl | methyl | methyl | methyl | methyl | methyl |
| 2-17-025 | N-tolyl | N-tolyl | b1 | H | b1 | H | H |
| 2-17-026 | N-tolyl | N-tolyl | b2 | H | b2 | H | H |
| 2-17-027 | N-tolyl | N-tolyl | b3 | H | b3 | H | H |
| 2-17-028 | N-tolyl | N-tolyl | t-bu | H | t-bu | H | t-bu |
| 2-17-029 | N-tolyl | N-tolyl | methyl | H | methyl | H | methyl |
| 2-17-030 | N-tolyl | N-tolyl | b1 | H | b1 | H | b1 |
| 2-17-031 | N-tolyl | N-tolyl | b2 | H | b2 | H | b2 |
| 2-17-032 | N-tolyl | N-tolyl | b3 | H | b3 | H | b3 |
| 2-17-033 | N-tolyl | N-tolyl | b1 | b1 | b1 | b1 | H |
| 2-17-034 | N-tolyl | N-tolyl | b2 | b2 | b2 | b2 | H |
| 2-17-035 | N-tolyl | N-tolyl | b3 | b3 | b3 | b3 | H |
| 2-17-036 | N-tolyl | N-tolyl | b1 | b1 | b1 | b1 | b1 |
| 2-17-037 | N-tolyl | N-tolyl | b2 | b2 | b2 | b2 | b2 |
| 2-17-038 | N-tolyl | N-tolyl | b3 | b3 | b3 | b3 | b3 |
| 2-17-039 | N-Biph | N-Biph | H | H | H | H | H |
| 2-17-040 | N-Biph | N-Biph | t-bu | H | t-bu | H | H |
| 2-17-041 | N-Biph | N-Biph | methyl | H | methyl | H | H |
| 2-17-042 | N-Biph | N-Biph | t-bu | t-bu | t-bu | t-bu | t-bu |
| 2-17-043 | N-Biph | N-Biph | methyl | methyl | methyl | methyl | methyl |
| 2-17-044 | N-Biph | N-Biph | b1 | H | b1 | H | H |
| 2-17-045 | N-Biph | N-Biph | b2 | H | b2 | H | H |
| 2-17-046 | N-Biph | N-Biph | b3 | H | b3 | H | H |
| 2-17-047 | N-Biph | N-Biph | t-bu | H | t-bu | H | t-bu |
| 2-17-048 | N-Biph | N-Biph | methyl | H | methyl | H | methyl |
| 2-17-049 | N-Biph | N-Biph | b1 | H | b1 | H | b1 |
| 2-17-050 | N-Biph | N-Biph | b2 | H | b2 | H | b2 |
| 2-17-051 | N-Biph | N-Biph | b3 | H | b3 | H | b3 |
| 2-17-052 | N-Biph | N-Biph | b1 | b1 | b1 | b1 | H |
| 2-17-053 | N-Biph | N-Biph | b2 | b2 | b2 | b2 | H |
| 2-17-054 | N-Biph | N-Biph | b3 | b3 | b3 | b3 | H |
| 2-17-055 | N-Biph | N-Biph | b1 | b1 | b1 | b1 | b1 |
| 2-17-056 | N-Biph | N-Biph | b2 | b2 | b2 | b2 | b2 |
| 2-17-057 | N-Biph | N-Biph | b3 | b3 | b3 | b3 | b3 |
| 2-17-058 | N—Ph | O | H | H | H | H | H |
| 2-17-059 | N—Ph | O | t-bu | H | t-bu | H | H |
| 2-17-060 | N—Ph | O | methyl | H | methyl | H | H |
| 2-17-061 | N—Ph | O | t-bu | t-bu | t-bu | t-bu | t-bu |
| 2-17-062 | N—Ph | O | methyl | methyl | methyl | methyl | methyl |
| 2-17-063 | N—Ph | O | b1 | H | b1 | H | H |
| 2-17-064 | N—Ph | O | b2 | H | b2 | H | H |
| 2-17-065 | N—Ph | O | b3 | H | b3 | H | H |
| 2-17-066 | N—Ph | O | t-bu | H | t-bu | H | t-bu |
| 2-17-067 | N—Ph | O | methyl | H | methyl | H | methyl |
| 2-17-068 | N—Ph | O | b1 | H | b1 | H | b1 |
| 2-17-069 | N—Ph | O | b2 | H | b2 | H | b2 |
| 2-17-070 | N—Ph | O | b3 | H | b3 | H | b3 |
| 2-17-071 | N—Ph | O | b1 | b1 | b1 | b1 | H |
| 2-17-072 | N—Ph | O | b2 | b2 | b2 | b2 | H |
| 2-17-073 | N—Ph | O | b3 | b3 | b3 | b3 | H |
| 2-17-074 | N—Ph | O | b1 | b1 | b1 | b1 | b1 |
| 2-17-075 | N—Ph | O | b2 | b2 | b2 | b2 | b2 |
| 2-17-076 | N—Ph | O | b3 | b3 | b3 | b3 | b3 |
| 2-17-077 | N—Ph | S | H | H | H | H | H |
| 2-17-078 | N—Ph | S | t-bu | H | t-bu | H | H |
| 2-17-079 | N—Ph | S | methyl | H | methyl | H | H |
| 2-17-080 | N—Ph | S | t-bu | t-bu | t-bu | t-bu | t-bu |
| 2-17-081 | N—Ph | S | methyl | methyl | methyl | methyl | methyl |
| 2-17-082 | N—Ph | S | b1 | H | b1 | H | H |
| 2-17-083 | N—Ph | S | b2 | H | b2 | H | H |
| 2-17-084 | N—Ph | S | b3 | H | b3 | H | H |
| 2-17-085 | N—Ph | S | t-bu | H | t-bu | H | t-bu |
| 2-17-086 | N—Ph | S | methyl | H | methyl | H | methyl |
| 2-17-087 | N—Ph | S | b1 | H | b1 | H | b1 |
| 2-17-088 | N—Ph | S | b2 | H | b2 | H | b2 |
| 2-17-089 | N—Ph | S | b3 | H | b3 | H | b3 |
| 2-17-090 | N—Ph | S | b1 | b1 | b1 | b1 | H |
| 2-17-091 | N—Ph | S | b2 | b2 | b2 | b2 | H |
| 2-17-092 | N—Ph | S | b3 | b3 | b3 | b3 | H |
| 2-17-093 | N—Ph | S | b1 | b1 | b1 | b1 | b1 |
| 2-17-094 | N—Ph | S | b2 | b2 | b2 | b2 | b2 |
| 2-17-095 | N—Ph | S | b3 | b3 | b3 | b3 | b3 |

### Group 2-18

The compounds are represented by the formula 2-18 below, wherein CY₂ and CY₃ are each a benzene group, X₁ is B, and Y1, Y2, Y3, R1b, (R2)a2, (R3)a3 are each shown in the table 18 below,

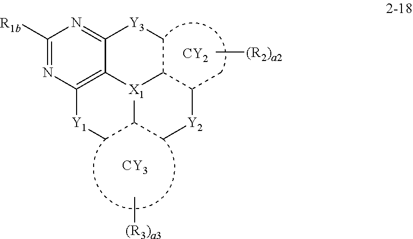

TABLE 18

| Compound | | | | | |
|---|---|---|---|---|---|
| No. | Y1/Y3 | Y2 | R1b | (R2)a2 | (R3)a3 |
| 2-18-001 | N—Ph | N—Ph | H | H | H |
| 2-18-002 | N—Ph | N—Ph | t-bu | H | H |
| 2-18-003 | N—Ph | N—Ph | methyl | H | H |
| 2-18-004 | N—Ph | N—Ph | t-bu | t-bu | t-bu |
| 2-18-005 | N—Ph | N—Ph | methyl | methyl | methyl |
| 2-18-006 | N—Ph | N—Ph | b1 | H | H |
| 2-18-007 | N—Ph | N—Ph | b2 | H | H |
| 2-18-008 | N—Ph | N—Ph | b3 | H | H |
| 2-18-009 | N—Ph | N—Ph | H | H | a1 |
| 2-18-010 | N—Ph | N—Ph | t-bu | H | a1 |
| 2-18-011 | N—Ph | N—Ph | methyl | H | a1 |
| 2-18-012 | N—Ph | N—Ph | b1 | H | a1 |
| 2-18-013 | N—Ph | N—Ph | b2 | H | a1 |
| 2-18-014 | N—Ph | N—Ph | b3 | H | a1 |
| 2-18-015 | N—Ph | N—Ph | H | H | a2 |
| 2-18-016 | N—Ph | N—Ph | t-bu | H | a2 |
| 2-18-017 | N—Ph | N—Ph | methyl | H | a2 |
| 2-18-018 | N—Ph | N—Ph | b1 | H | a2 |
| 2-18-019 | N—Ph | N—Ph | b2 | H | a2 |
| 2-18-020 | N—Ph | N—Ph | b3 | H | a2 |
| 2-18-021 | N—Ph | N—Ph | H | H | a3 |
| 2-18-022 | N—Ph | N—Ph | t-bu | H | a3 |
| 2-18-023 | N—Ph | N—Ph | methyl | H | a3 |
| 2-18-024 | N—Ph | N—Ph | b1 | H | a3 |
| 2-18-025 | N—Ph | N—Ph | b2 | H | a3 |
| 2-18-026 | N—Ph | N—Ph | b3 | H | a3 |
| 2-18-027 | N—Ph | N—Ph | H | H | a4 |
| 2-18-028 | N—Ph | N—Ph | t-bu | H | a4 |
| 2-18-029 | N—Ph | N—Ph | methyl | H | a4 |
| 2-18-030 | N—Ph | N—Ph | b1 | H | a4 |
| 2-18-031 | N—Ph | N—Ph | b2 | H | a4 |
| 2-18-032 | N—Ph | N—Ph | b3 | H | a4 |
| 2-18-033 | N—Ph | N—Ph | H | H | a5 |
| 2-18-034 | N—Ph | N—Ph | t-bu | H | a5 |
| 2-18-035 | N—Ph | N—Ph | methyl | H | a5 |
| 2-18-036 | N—Ph | N—Ph | b1 | H | a5 |
| 2-18-037 | N—Ph | N—Ph | b2 | H | a5 |
| 2-18-038 | N—Ph | N—Ph | b3 | H | a5 |
| 2-18-039 | N Ph | N—Ph | H | H | a6 |
| 2-18-040 | N—Ph | N—Ph | t-bu | H | a6 |
| 2-18-041 | N—Ph | N—Ph | methyl | H | a6 |
| 2-18-042 | N—Ph | N—Ph | b1 | H | a6 |
| 2-18-043 | N—Ph | N—Ph | b2 | H | a6 |
| 2-18-044 | N—Ph | N—Ph | b3 | H | a6 |
| 2-18-045 | N—Ph | N—Ph | H | H | b1 |
| 2-18-046 | N—Ph | N—Ph | t-bu | H | b1 |
| 2-18-047 | N—Ph | N—Ph | methyl | H | b1 |
| 2-18-048 | N—Ph | N—Ph | b1 | H | b1 |
| 2-18-049 | N—Ph | N—Ph | b2 | H | b1 |
| 2-18-050 | N—Ph | N—Ph | b3 | H | b1 |
| 2-18-051 | N—Ph | N—Ph | H | H | b2 |
| 2-18-052 | N—Ph | N—Ph | t-bu | H | b2 |
| 2-18-053 | N—Ph | N—Ph | methyl | H | b2 |
| 2-18-054 | N—Ph | N—Ph | b1 | H | b2 |
| 2-18-055 | N—Ph | N—Ph | b2 | H | b2 |
| 2-18-056 | N—Ph | N—Ph | b3 | H | b2 |
| 2-18-057 | N—Ph | N—Ph | H | H | b3 |
| 2-18-058 | N—Ph | N—Ph | t-bu | H | b3 |
| 2-18-059 | N—Ph | N—Ph | methyl | H | b3 |
| 2-18-060 | N—Ph | N—Ph | b1 | H | b3 |
| 2-18-061 | N—Ph | N—Ph | b2 | H | b3 |
| 2-18-062 | N—Ph | N—Ph | b3 | H | b3 |
| 2-18-063 | N—Ph | N—Ph | H | H | b4 |
| 2-18-064 | N—Ph | N—Ph | t-bu | H | b4 |
| 2-18-065 | N—Ph | N—Ph | methyl | H | b4 |
| 2-18-066 | N—Ph | N—Ph | b1 | H | b4 |
| 2-18-067 | N—Ph | N—Ph | b2 | H | b4 |
| 2-18-068 | N—Ph | N—Ph | b3 | H | b4 |
| 2-18-069 | N—Ph | N—Ph | H | H | b5 |
| 2-18-070 | N—Ph | N—Ph | t-bu | H | b5 |
| 2-18-071 | N—Ph | N—Ph | methyl | H | b5 |
| 2-18-072 | N—Ph | N—Ph | b1 | H | b5 |
| 2-18-073 | N—Ph | N—Ph | b2 | H | b5 |
| 2-18-074 | N—Ph | N—Ph | b3 | H | b5 |
| 2-18-075 | N—Ph | N—Ph | H | H | b6 |
| 2-18-076 | N—Ph | N—Ph | t-bu | H | b6 |
| 2-18-077 | N—Ph | N—Ph | methyl | H | b6 |
| 2-18-078 | N—Ph | N—Ph | b1 | H | b6 |
| 2-18-079 | N—Ph | N—Ph | b2 | H | b6 |
| 2-18-080 | N—Ph | N—Ph | b3 | H | b6 |
| 2-18-081 | N—Ph | N—Ph | H | H | b7 |
| 2-18-082 | N—Ph | N—Ph | t-bu | H | b7 |
| 2-18-083 | N—Ph | N—Ph | methyl | H | b7 |
| 2-18-084 | N—Ph | N—Ph | b1 | H | b7 |
| 2-18-085 | N—Ph | N—Ph | b2 | H | b7 |
| 2-18-086 | N—Ph | N—Ph | b3 | H | b7 |
| 2-18-087 | N—Ph | N—Ph | H | H | b8 |
| 2-18-088 | N—Ph | N—Ph | t-bu | H | b8 |
| 2-18-089 | N—Ph | N—Ph | methyl | H | b8 |
| 2-18-090 | N—Ph | N—Ph | b1 | H | b8 |
| 2-18-091 | N—Ph | N—Ph | b2 | H | b8 |
| 2-18-092 | N—Ph | N—Ph | b3 | H | b8 |
| 2-18-093 | N—Ph | N—Ph | H | H | b9 |
| 2-18-094 | N—Ph | N—Ph | t-bu | H | b9 |
| 2-18-095 | N—Ph | N—Ph | methyl | H | b9 |
| 2-18-096 | N—Ph | N—Ph | b1 | H | b9 |
| 2-18-097 | N—Ph | N—Ph | b2 | H | b9 |
| 2-18-098 | N—Ph | N—Ph | b3 | H | b9 |
| 2-18-099 | N—Ph | N—Ph | H | H | b10 |
| 2-18-100 | N—Ph | N—Ph | t-bu | H | b10 |
| 2-18-101 | N—Ph | N—Ph | methyl | H | b10 |
| 2-18-102 | N—Ph | N—Ph | b1 | H | b10 |
| 2-18-103 | N—Ph | N—Ph | b2 | H | b10 |
| 2-18-104 | N—Ph | N—Ph | b3 | H | b10 |
| 2-18-105 | N—Ph | N—Ph | H | a1 | H |
| 2-18-106 | N—Ph | N—Ph | t-bu | a1 | H |
| 2-18-107 | N—Ph | N—Ph | methyl | a1 | H |
| 2-18-108 | N—Ph | N—Ph | b1 | a1 | H |
| 2-18-109 | N—Ph | N—Ph | b2 | a1 | H |
| 2-18-110 | N—Ph | N—Ph | b3 | a1 | H |
| 2-18-111 | N—Ph | N—Ph | H | a2 | H |
| 2-18-112 | N—Ph | N—Ph | t-bu | a2 | H |
| 2-18-113 | N—Ph | N—Ph | methyl | a2 | H |
| 2-18-114 | N—Ph | N—Ph | b1 | a2 | H |
| 2-18-115 | N—Ph | N—Ph | b2 | a2 | H |
| 2-18-116 | N—Ph | N—Ph | b3 | a2 | H |
| 2-18-117 | N—Ph | N—Ph | H | a3 | H |
| 2-18-118 | N—Ph | N—Ph | t-bu | a3 | H |
| 2-18-119 | N—Ph | N—Ph | methyl | a3 | H |
| 2-18-120 | N—Ph | N—Ph | b1 | a3 | H |
| 2-18-121 | N—Ph | N—Ph | b2 | a3 | H |
| 2-18-122 | N—Ph | N—Ph | b3 | a3 | H |
| 2-18-123 | N—Ph | N—Ph | H | a4 | H |
| 2-18-124 | N—Ph | N—Ph | t-bu | a4 | H |
| 2-18-125 | N—Ph | N—Ph | methyl | a4 | H |
| 2-18-126 | N—Ph | N—Ph | b1 | a4 | H |
| 2-18-127 | N—Ph | N—Ph | b2 | a4 | H |
| 2-18-128 | N—Ph | N—Ph | b3 | a4 | H |
| 2-18-129 | N—Ph | N—Ph | H | a5 | H |
| 2-18-130 | N—Ph | N—Ph | t-bu | a5 | H |
| 2-18-131 | N—Ph | N—Ph | methyl | a5 | H |
| 2-18-132 | N—Ph | N—Ph | b1 | a5 | H |
| 2-18-133 | N—Ph | N—Ph | b2 | a5 | H |
| 2-18-134 | N—Ph | N—Ph | b3 | a5 | H |
| 2-18-135 | N—Ph | N—Ph | H | a6 | H |
| 2-18-136 | N—Ph | N—Ph | t-bu | a6 | H |
| 2-18-137 | N—Ph | N—Ph | methyl | a6 | H |
| 2-18-138 | N—Ph | N—Ph | b1 | a6 | H |
| 2-18-139 | N—Ph | N—Ph | b2 | a6 | H |
| 2-18-140 | N—Ph | N—Ph | b3 | a6 | H |
| 2-18-141 | N—Ph | N—Ph | H | b1 | H |
| 2-18-142 | N—Ph | N—Ph | t-bu | b1 | H |
| 2-18-143 | N—Ph | N—Ph | methyl | b1 | H |
| 2-18-144 | N—Ph | N—Ph | b1 | b1 | H |
| 2-18-145 | N—Ph | N—Ph | b2 | b1 | H |
| 2-18-146 | N—Ph | N—Ph | b3 | b1 | H |
| 2-18-147 | N—Ph | N—Ph | H | b2 | H |
| 2-18-148 | N—Ph | N—Ph | t-bu | b2 | H |
| 2-18-149 | N—Ph | N—Ph | methyl | b2 | H |
| 2-18-150 | N—Ph | N—Ph | b1 | b2 | H |
| 2-18-151 | N—Ph | N—Ph | b2 | b2 | H |
| 2-18-152 | N—Ph | N—Ph | b3 | b2 | H |
| 2-18-153 | N—Ph | N—Ph | H | b3 | H |
| 2-18-154 | N—Ph | N—Ph | t-bu | b3 | H |
| 2-18-155 | N—Ph | N—Ph | methyl | b3 | H |
| 2-18-156 | N—Ph | N—Ph | b1 | b3 | H |
| 2-18-157 | N—Ph | N—Ph | b2 | b3 | H |
| 2-18-158 | N—Ph | N—Ph | b3 | b3 | H |
| 2-18-159 | N—Ph | N—Ph | H | b4 | H |
| 2-18-160 | N—Ph | N—Ph | t-bu | b4 | H |
| 2-18-161 | N—Ph | N—Ph | methyl | b4 | H |
| 2-18-162 | N—Ph | N—Ph | b1 | b4 | H |
| 2-18-163 | N—Ph | N—Ph | b2 | b4 | H |
| 2-18-164 | N—Ph | N—Ph | b3 | b4 | H |
| 2-18-165 | N—Ph | N—Ph | H | b5 | H |
| 2-18-166 | N—Ph | N—Ph | t-bu | b5 | H |
| 2-18-167 | N—Ph | N—Ph | methyl | b5 | H |
| 2-18-168 | N—Ph | N—Ph | b1 | b5 | H |
| 2-18-169 | N—Ph | N—Ph | b2 | b5 | H |
| 2-18-170 | N—Ph | N—Ph | b3 | b5 | H |
| 2-18-171 | N—Ph | N—Ph | H | b6 | H |
| 2-18-172 | N—Ph | N—Ph | t-bu | b6 | H |
| 2-18-173 | N—Ph | N—Ph | methyl | b6 | H |
| 2-18-174 | N—Ph | N—Ph | b1 | b6 | H |
| 2-18-175 | N—Ph | N—Ph | b2 | b6 | H |
| 2-18-176 | N—Ph | N—Ph | b3 | b6 | H |
| 2-18-177 | N—Ph | N—Ph | H | b7 | H |
| 2-18-178 | N—Ph | N—Ph | t-bu | b7 | H |
| 2-18-179 | N—Ph | N—Ph | methyl | b7 | H |
| 2-18-180 | N—Ph | N—Ph | b1 | b7 | H |
| 2-18-181 | N—Ph | N—Ph | b2 | b7 | H |
| 2-18-182 | N—Ph | N—Ph | b3 | b7 | H |
| 2-18-183 | N—Ph | N—Ph | H | b8 | H |
| 2-18-184 | N—Ph | N—Ph | t-bu | b8 | H |
| 2-18-185 | N—Ph | N—Ph | methyl | b8 | H |
| 2-18-186 | N—Ph | N—Ph | b1 | b8 | H |
| 2-18-187 | N—Ph | N—Ph | b2 | b8 | H |
| 2-18-188 | N—Ph | N—Ph | b3 | b8 | H |
| 2-18-189 | N—Ph | N—Ph | H | b9 | H |
| 2-18-190 | N—Ph | N—Ph | t-bu | b9 | H |
| 2-18-191 | N—Ph | N—Ph | methyl | b9 | H |
| 2-18-192 | N—Ph | N—Ph | b1 | b9 | H |
| 2-18-193 | N—Ph | N—Ph | b2 | b9 | H |
| 2-18-194 | N—Ph | N—Ph | b3 | b9 | H |
| 2-18-195 | N—Ph | N—Ph | H | b10 | H |
| 2-18-196 | N—Ph | N—Ph | t-bu | b10 | H |
| 2-18-197 | N—Ph | N—Ph | methyl | b10 | H |
| 2-18-198 | N—Ph | N—Ph | b1 | b10 | H |
| 2-18-199 | N—Ph | N—Ph | b2 | b10 | H |
| 2-18-200 | N—Ph | N—Ph | b3 | b10 | H |
| 2-18-201 | N—Ph | N—Ph | H | a1 | a1 |
| 2-18-202 | N—Ph | N—Ph | t-bu | a1 | a1 |
| 2-18-203 | N—Ph | N—Ph | methyl | a1 | a1 |
| 2-18-204 | N—Ph | N—Ph | b1 | a1 | a1 |
| 2-18-205 | N—Ph | N—Ph | b2 | a1 | a1 |
| 2-18-206 | N—Ph | N—Ph | b3 | a1 | a1 |
| 2-18-207 | N—Ph | N—Ph | H | a2 | a2 |
| 2-18-208 | N—Ph | N—Ph | t-bu | a2 | a2 |
| 2-18-209 | N—Ph | N—Ph | methyl | a2 | a2 |
| 2-18-210 | N—Ph | N—Ph | b1 | a2 | a2 |
| 2-18-211 | N—Ph | N—Ph | b2 | a2 | a2 |
| 2-18-212 | N—Ph | N—Ph | b3 | a2 | a2 |
| 2-18-213 | N—Ph | N—Ph | H | a3 | a3 |
| 2-18-214 | N—Ph | N—Ph | t-bu | a3 | a3 |
| 2-18-215 | N—Ph | N—Ph | methyl | a3 | a3 |
| 2-18-216 | N—Ph | N—Ph | b1 | a3 | a3 |
| 2-18-217 | N—Ph | N—Ph | b2 | a3 | a3 |
| 2-18-218 | N—Ph | N—Ph | b3 | a3 | a3 |
| 2-18-219 | N—Ph | N—Ph | H | b1 | b1 |
| 2-18-220 | N—Ph | N—Ph | t-bu | b1 | b1 |
| 2-18-221 | N—Ph | N—Ph | methyl | b1 | b1 |
| 2-18-222 | N—Ph | N—Ph | b1 | b1 | b1 |
| 2-18-223 | N—Ph | N—Ph | b2 | b1 | b1 |
| 2-18-224 | N—Ph | N—Ph | b3 | b1 | b1 |
| 2-18-225 | N—Ph | N—Ph | H | b2 | b2 |
| 2-18-226 | N—Ph | N—Ph | t-bu | b2 | b2 |
| 2-18-227 | N—Ph | N—Ph | methyl | b2 | b2 |
| 2-18-228 | N—Ph | N—Ph | b1 | b2 | b2 |
| 2-18-229 | N—Ph | N—Ph | b2 | b2 | b2 |
| 2-18-230 | N—Ph | N—Ph | b3 | b2 | b2 |
| 2-18-231 | N—Ph | N—Ph | H | b3 | b3 |
| 2-18-232 | N—Ph | N—Ph | t-bu | b3 | b3 |
| 2-18-233 | N—Ph | N—Ph | methyl | b3 | b3 |
| 2-18-234 | N—Ph | N—Ph | b1 | b3 | b3 |
| 2-18-235 | N—Ph | N—Ph | b2 | b3 | b3 |
| 2-18-236 | N—Ph | N—Ph | b3 | b3 | b3 |
| 2-18-237 | N—Ph | N—Ph | H | b5 | b5 |
| 2-18-238 | N—Ph | N—Ph | t-bu | b5 | b5 |
| 2-18-239 | N—Ph | N—Ph | methyl | b5 | b5 |
| 2-18-240 | N—Ph | N—Ph | b1 | b5 | b5 |
| 2-18-241 | N—Ph | N—Ph | b2 | b5 | b5 |
| 2-18-242 | N—Ph | N—Ph | b3 | b5 | b5 |
| 2-18-243 | N—Ph | N—Ph | H | b6 | b6 |
| 2-18-244 | N—Ph | N—Ph | t-bu | b6 | b6 |
| 2-18-245 | N—Ph | N—Ph | methyl | b6 | b6 |
| 2-18-246 | N—Ph | N—Ph | b1 | b6 | b6 |
| 2-18-247 | N—Ph | N—Ph | b2 | b6 | b6 |
| 2-18-248 | N—Ph | N—Ph | b3 | b6 | b6 |
| 2-18-249 | N—Ph | N—Ph | H | b7 | b7 |
| 2-18-250 | N—Ph | N—Ph | t-bu | b7 | b7 |
| 2-18-251 | N—Ph | N—Ph | methyl | b7 | b7 |
| 2-18-252 | N—Ph | N—Ph | b1 | b7 | b7 |
| 2-18-253 | N—Ph | N—Ph | b2 | b7 | b7 |
| 2-18-254 | N—Ph | N—Ph | b3 | b7 | b7 |
| 2-18-255 | N-tolyl | N-tolyl | H | H | H |
| 2-18-256 | N-tolyl | N-tolyl | t-bu | H | H |
| 2-18-257 | N-tolyl | N-tolyl | methyl | H | H |
| 2-18-258 | N-tolyl | N-tolyl | t-bu | t-bu | t-bu |
| 2-18-259 | N-tolyl | N-tolyl | methyl | methyl | methyl |
| 2-18-260 | N-tolyl | N-tolyl | b1 | H | H |
| 2-18-261 | N-tolyl | N-tolyl | b2 | H | H |
| 2-18-262 | N-tolyl | N-tolyl | b3 | H | H |
| 2-18-263 | N-tolyl | N-tolyl | H | H | a1 |
| 2-18-264 | N-tolyl | N-tolyl | t-bu | H | a1 |
| 2-18-265 | N-tolyl | N-tolyl | methyl | H | a1 |
| 2-18-266 | N-tolyl | N-tolyl | b1 | H | a1 |
| 2-18-267 | N-tolyl | N-tolyl | b2 | H | a1 |
| 2-18-268 | N-tolyl | N-tolyl | b3 | H | a1 |
| 2-18-269 | N-tolyl | N-tolyl | H | H | a2 |
| 2-18-270 | N-tolyl | N-tolyl | t-bu | H | a2 |
| 2-18-271 | N-tolyl | N-tolyl | methyl | H | a2 |
| 2-18-272 | N-tolyl | N-tolyl | b1 | H | a2 |
| 2-18-273 | N-tolyl | N-tolyl | b2 | H | a2 |
| 2-18-274 | N-tolyl | N-tolyl | b3 | H | a2 |
| 2-18-275 | N-tolyl | N-tolyl | H | H | a3 |
| 2-18-276 | N-tolyl | N-tolyl | t-bu | H | a3 |
| 2-18-277 | N-tolyl | N-tolyl | methyl | H | a3 |
| 2-18-278 | N-tolyl | N-tolyl | b1 | H | a3 |
| 2-18-279 | N-tolyl | N-tolyl | b2 | H | a3 |
| 2-18-280 | N-tolyl | N-tolyl | b3 | H | a3 |
| 2-18-281 | N-tolyl | N-tolyl | H | H | a4 |
| 2-18-282 | N-tolyl | N-tolyl | t-bu | H | a4 |
| 2-18-283 | N-tolyl | N-tolyl | methyl | H | a4 |
| 2-18-284 | N-tolyl | N-tolyl | b1 | H | a4 |
| 2-18-285 | N-tolyl | N-tolyl | b2 | H | a4 |
| 2-18-286 | N-tolyl | N-tolyl | b3 | H | a4 |
| 2-18-287 | N-tolyl | N-tolyl | H | H | a5 |
| 2-18-288 | N-tolyl | N-tolyl | t-bu | H | a5 |
| 2-18-289 | N-tolyl | N-tolyl | methyl | H | a5 |
| 2-18-290 | N-tolyl | N-tolyl | b1 | H | a5 |
| 2-18-291 | N-tolyl | N-tolyl | b2 | H | a5 |
| 2-18-292 | N-tolyl | N-tolyl | b3 | H | a5 |
| 2-18-293 | N-tolyl | N-tolyl | H | H | a6 |
| 2-18-294 | N-tolyl | N-tolyl | t-bu | H | a6 |
| 2-18-295 | N-tolyl | N-tolyl | methyl | H | a6 |
| 2-18-296 | N-tolyl | N-tolyl | b1 | H | a6 |
| 2-18-297 | N-tolyl | N-tolyl | b2 | H | a6 |
| 2-18-298 | N-tolyl | N-tolyl | b3 | H | a6 |
| 2-18-299 | N-tolyl | N-tolyl | H | H | b1 |
| 2-18-300 | N-tolyl | N-tolyl | t-bu | H | b1 |
| 2-18-301 | N-tolyl | N-tolyl | methyl | H | b1 |
| 2-18-302 | N-tolyl | N-tolyl | b1 | H | b1 |
| 2-18-303 | N-tolyl | N-tolyl | b2 | H | b1 |
| 2-18-304 | N-tolyl | N-tolyl | b3 | H | b1 |
| 2-18-305 | N-tolyl | N-tolyl | H | H | b2 |
| 2-18-306 | N-tolyl | N-tolyl | t-bu | H | b2 |
| 2-18-307 | N-tolyl | N-tolyl | methyl | H | b2 |
| 2-18-308 | N-tolyl | N-tolyl | b1 | H | b2 |
| 2-18-309 | N-tolyl | N-tolyl | b2 | H | b2 |
| 2-18-310 | N-tolyl | N-tolyl | b3 | H | b2 |
| 2-18-311 | N-tolyl | N-tolyl | H | H | b3 |
| 2-18-312 | N-tolyl | N-tolyl | t-bu | H | b3 |
| 2-18-313 | N-tolyl | N-tolyl | methyl | H | b3 |
| 2-18-314 | N-tolyl | N-tolyl | b1 | H | b3 |
| 2-18-315 | N-tolyl | N-tolyl | b2 | H | b3 |
| 2-18-316 | N-tolyl | N-tolyl | b3 | H | b3 |
| 2-18-317 | N-tolyl | N-tolyl | H | H | b4 |
| 2-18-318 | N-tolyl | N-tolyl | t-bu | H | b4 |
| 2-18-319 | N-tolyl | N-tolyl | methyl | H | b4 |
| 2-18-320 | N-tolyl | N-tolyl | b1 | H | b4 |
| 2-18-321 | N-tolyl | N-tolyl | b2 | H | b4 |
| 2-18-322 | N-tolyl | N-tolyl | b3 | H | b4 |
| 2-18-323 | N-tolyl | N-tolyl | H | H | b5 |
| 2-18-324 | N-tolyl | N-tolyl | t-bu | H | b5 |
| 2-18-325 | N-tolyl | N-tolyl | methyl | H | b5 |
| 2-18-326 | N-tolyl | N-tolyl | b1 | H | b5 |
| 2-18-327 | N-tolyl | N-tolyl | b2 | H | b5 |
| 2-18-328 | N-tolyl | N-tolyl | b3 | H | b5 |
| 2-18-329 | N-tolyl | N-tolyl | H | H | b6 |
| 2-18-330 | N-tolyl | N-tolyl | t-bu | H | b6 |
| 2-18-331 | N-tolyl | N-tolyl | methyl | H | b6 |
| 2-18-332 | N-tolyl | N-tolyl | b1 | H | b6 |
| 2-18-333 | N-tolyl | N-tolyl | b2 | H | b6 |
| 2-18-334 | N-tolyl | N-tolyl | b3 | H | b6 |
| 2-18-335 | N-tolyl | N-tolyl | H | H | b7 |
| 2-18-336 | N-tolyl | N-tolyl | t-bu | H | b7 |
| 2-18-337 | N-tolyl | N-tolyl | methyl | H | b7 |
| 2-18-338 | N-tolyl | N-tolyl | b1 | H | b7 |
| 2-18-339 | N-tolyl | N-tolyl | b2 | H | b7 |
| 2-18-340 | N-tolyl | N-tolyl | b3 | H | b7 |
| 2-18-341 | N-tolyl | N-tolyl | H | H | b8 |
| 2-18-342 | N-tolyl | N-tolyl | t-bu | H | b8 |
| 2-18-343 | N-tolyl | N-tolyl | methyl | H | b8 |
| 2-18-344 | N-tolyl | N-tolyl | b1 | H | b8 |
| 2-18-345 | N-tolyl | N-tolyl | b2 | H | b8 |
| 2-18-346 | N-tolyl | N-tolyl | b3 | H | b8 |
| 2-18-347 | N-tolyl | N-tolyl | H | H | b9 |
| 2-18-348 | N-tolyl | N-tolyl | t-bu | H | b9 |
| 2-18-349 | N-tolyl | N-tolyl | methyl | H | b9 |
| 2-18-350 | N-tolyl | N-tolyl | b1 | H | b9 |
| 2-18-351 | N-tolyl | N-tolyl | b2 | H | b9 |
| 2-18-352 | N-tolyl | N-tolyl | b3 | H | b9 |
| 2-18-353 | N-tolyl | N-tolyl | H | H | b10 |
| 2-18-354 | N-tolyl | N-tolyl | t-bu | H | b10 |
| 2-18-355 | N-tolyl | N-tolyl | methyl | H | b10 |
| 2-18-356 | N-tolyl | N-tolyl | b1 | H | b10 |
| 2-18-357 | N-tolyl | N-tolyl | b2 | H | b10 |
| 2-18-358 | N-tolyl | N-tolyl | b3 | H | b10 |
| 2-18-359 | N-tolyl | N-tolyl | H | a1 | H |
| 2-18-360 | N-tolyl | N-tolyl | t-bu | a1 | H |
| 2-18-361 | N-tolyl | N-tolyl | methyl | a1 | H |
| 2-18-362 | N-tolyl | N-tolyl | b1 | a1 | H |
| 2-18-363 | N-tolyl | N-tolyl | b2 | a1 | H |
| 2-18-364 | N-tolyl | N-tolyl | b3 | a1 | H |
| 2-18-365 | N-tolyl | N-tolyl | H | a2 | H |
| 2-18-366 | N-tolyl | N-tolyl | t-bu | a2 | H |
| 2-18-367 | N-tolyl | N-tolyl | methyl | a2 | H |
| 2-18-368 | N-tolyl | N-tolyl | b1 | a2 | H |
| 2-18-369 | N-tolyl | N-tolyl | b2 | a2 | H |
| 2-18-370 | N-tolyl | N-tolyl | b3 | a2 | H |
| 2-18-371 | N-tolyl | N-tolyl | H | a3 | H |
| 2-18-372 | N-tolyl | N-tolyl | t-bu | a3 | H |
| 2-18-373 | N-tolyl | N-tolyl | methyl | a3 | H |
| 2-18-374 | N-tolyl | N-tolyl | b1 | a3 | H |
| 2-18-375 | N-tolyl | N-tolyl | b2 | a3 | H |
| 2-18-376 | N-tolyl | N-tolyl | b3 | a3 | H |
| 2-18-377 | N-tolyl | N-tolyl | H | a4 | H |
| 2-18-378 | N-tolyl | N-tolyl | t-bu | a4 | H |
| 2-18-379 | N-tolyl | N-tolyl | methyl | a4 | H |
| 2-18-380 | N-tolyl | N-tolyl | b1 | a4 | H |
| 2-18-381 | N-tolyl | N-tolyl | b2 | a4 | H |
| 2-18-382 | N-tolyl | N-tolyl | b3 | a4 | H |
| 2-18-383 | N-tolyl | N-tolyl | H | a5 | H |
| 2-18-384 | N-tolyl | N-tolyl | t-bu | a5 | H |
| 2-18-385 | N-tolyl | N-tolyl | methyl | a5 | H |
| 2-18-386 | N-tolyl | N-tolyl | b1 | a5 | H |
| 2-18-387 | N-tolyl | N-tolyl | b2 | a5 | H |
| 2-18-388 | N-tolyl | N-tolyl | b3 | a5 | H |
| 2-18-389 | N-tolyl | N-tolyl | H | a6 | H |
| 2-18-390 | N-tolyl | N-tolyl | t-bu | a6 | H |
| 2-18-391 | N-tolyl | N-tolyl | methyl | a6 | H |
| 2-18-392 | N-tolyl | N-tolyl | b1 | a6 | H |
| 2-18-393 | N-tolyl | N-tolyl | b2 | a6 | H |
| 2-18-394 | N-tolyl | N-tolyl | b3 | a6 | H |
| 2-18-395 | N-tolyl | N-tolyl | H | b1 | H |
| 2-18-396 | N-tolyl | N-tolyl | t-bu | b1 | H |
| 2-18-397 | N-tolyl | N-tolyl | methyl | b1 | H |
| 2-18-398 | N-tolyl | N-tolyl | b1 | b1 | H |
| 2-18-399 | N-tolyl | N-tolyl | b2 | b1 | H |
| 2-18-400 | N-tolyl | N-tolyl | b3 | b1 | H |
| 2-18-401 | N-tolyl | N-tolyl | H | b2 | H |
| 2-18-402 | N-tolyl | N-tolyl | t-bu | b2 | H |
| 2-18-403 | N-tolyl | N-tolyl | methyl | b2 | H |
| 2-18-404 | N-tolyl | N-tolyl | b1 | b2 | H |
| 2-18-405 | N-tolyl | N-tolyl | b2 | b2 | H |
| 2-18-406 | N-tolyl | N-tolyl | b3 | b2 | H |
| 2-18-407 | N-tolyl | N-tolyl | H | b3 | H |
| 2-18-408 | N-tolyl | N-tolyl | t-bu | b3 | H |
| 2-18-409 | N-tolyl | N-tolyl | methyl | b3 | H |
| 2-18-410 | N-tolyl | N-tolyl | b1 | b3 | H |
| 2-18-411 | N-tolyl | N-tolyl | b2 | b3 | H |
| 2-18-412 | N-tolyl | N-tolyl | b3 | b3 | H |
| 2-18-413 | N-tolyl | N-tolyl | H | b4 | H |
| 2-18-414 | N-tolyl | N-tolyl | t-bu | b4 | H |
| 2-18-415 | N-tolyl | N-tolyl | methyl | b4 | H |
| 2-18-416 | N-tolyl | N-tolyl | b1 | b4 | H |
| 2-18-417 | N-tolyl | N-tolyl | b2 | b4 | H |
| 2-18-418 | N-tolyl | N-tolyl | b3 | b4 | H |
| 2-18-419 | N-tolyl | N-tolyl | H | b5 | H |
| 2-18-420 | N-tolyl | N-tolyl | t-bu | b5 | H |
| 2-18-421 | N-tolyl | N-tolyl | methyl | b5 | H |
| 2-18-422 | N-tolyl | N-tolyl | b1 | b5 | H |
| 2-18-423 | N-tolyl | N-tolyl | b2 | b5 | H |
| 2-18-424 | N-tolyl | N-tolyl | b3 | b5 | H |
| 2-18-425 | N-tolyl | N-tolyl | H | b6 | H |
| 2-18-426 | N-tolyl | N-tolyl | t-bu | b6 | H |
| 2-18-427 | N-tolyl | N-tolyl | methyl | b6 | H |
| 2-18-428 | N-tolyl | N-tolyl | b1 | b6 | H |
| 2-18-429 | N-tolyl | N-tolyl | b2 | b6 | H |
| 2-18-430 | N-tolyl | N-tolyl | b3 | b6 | H |
| 2-18-431 | N-tolyl | N-tolyl | H | b7 | H |
| 2-18-432 | N-tolyl | N-tolyl | t-bu | b7 | H |
| 2-18-433 | N-tolyl | N-tolyl | methyl | b7 | H |
| 2-18-434 | N-tolyl | N-tolyl | b1 | b7 | H |
| 2-18-435 | N-tolyl | N-tolyl | b2 | b7 | H |
| 2-18-436 | N-tolyl | N-tolyl | b3 | b7 | H |
| 2-18-437 | N-tolyl | N-tolyl | H | b8 | H |
| 2-18-438 | N-tolyl | N-tolyl | t-bu | b8 | H |
| 2-18-439 | N-tolyl | N-tolyl | methyl | b8 | H |
| 2-18-440 | N-tolyl | N-tolyl | b1 | b8 | H |
| 2-18-441 | N-tolyl | N-tolyl | b2 | b8 | H |
| 2-18-442 | N-tolyl | N-tolyl | b3 | b8 | H |
| 2-18-443 | N-tolyl | N-tolyl | H | b9 | H |
| 2-18-444 | N-tolyl | N-tolyl | t-bu | b9 | H |
| 2-18-445 | N-tolyl | N-tolyl | methyl | b9 | H |
| 2-18-446 | N-tolyl | N-tolyl | b1 | b9 | H |
| 2-18-447 | N-tolyl | N-tolyl | b2 | b9 | H |
| 2-18-448 | N-tolyl | N-tolyl | b3 | b9 | H |
| 2-18-449 | N-tolyl | N-tolyl | H | b10 | H |
| 2-18-450 | N-tolyl | N-tolyl | t-bu | b10 | H |
| 2-18-451 | N-tolyl | N-tolyl | methyl | b10 | H |
| 2-18-452 | N-tolyl | N-tolyl | b1 | b10 | H |
| 2-18-453 | N-tolyl | N-tolyl | b2 | b10 | H |
| 2-18-454 | N-tolyl | N-tolyl | b3 | b10 | H |
| 2-18-455 | N-tolyl | N-tolyl | H | a1 | a1 |
| 2-18-456 | N-tolyl | N-tolyl | t-bu | a1 | a1 |
| 2-18-457 | N-tolyl | N-tolyl | methyl | a1 | a1 |
| 2-18-458 | N-tolyl | N-tolyl | b1 | a1 | a1 |
| 2-18-459 | N-tolyl | N-tolyl | b2 | a1 | a1 |
| 2-18-460 | N-tolyl | N-tolyl | b3 | a1 | a1 |
| 2-18-461 | N-tolyl | N-tolyl | H | a2 | a2 |
| 2-18-462 | N-tolyl | N-tolyl | t-bu | a2 | a2 |
| 2-18-463 | N-tolyl | N-tolyl | methyl | a2 | a2 |
| 2-18-464 | N-tolyl | N-tolyl | b1 | a2 | a2 |
| 2-18-465 | N-tolyl | N-tolyl | b2 | a2 | a2 |
| 2-18-466 | N-tolyl | N-tolyl | b3 | a2 | a2 |
| 2-18-467 | N-tolyl | N-tolyl | H | a3 | a3 |
| 2-18-468 | N-tolyl | N-tolyl | t-bu | a3 | a3 |
| 2-18-469 | N-tolyl | N-tolyl | methyl | a3 | a3 |
| 2-18-470 | N-tolyl | N-tolyl | b1 | a3 | a3 |
| 2-18-471 | N-tolyl | N-tolyl | b2 | a3 | a3 |
| 2-18-472 | N-tolyl | N-tolyl | b3 | a3 | a3 |
| 2-18-473 | N-tolyl | N-tolyl | H | b1 | b1 |
| 2-18-474 | N-tolyl | N-tolyl | t-bu | b1 | b1 |
| 2-18-475 | N-tolyl | N-tolyl | methyl | b1 | b1 |
| 2-18-476 | N-tolyl | N-tolyl | b1 | b1 | b1 |
| 2-18-477 | N-tolyl | N-tolyl | b2 | b1 | b1 |
| 2-18-478 | N-tolyl | N-tolyl | b3 | b1 | b1 |
| 2-18-479 | N-tolyl | N-tolyl | H | b2 | b2 |
| 2-18-480 | N-tolyl | N-tolyl | t-bu | b2 | b2 |
| 2-18-481 | N-tolyl | N-tolyl | methyl | b2 | b2 |
| 2-18-482 | N-tolyl | N-tolyl | b1 | b2 | b2 |
| 2-18-483 | N-tolyl | N-tolyl | b2 | b2 | b2 |
| 2-18-484 | N-tolyl | N-tolyl | b3 | b2 | b2 |
| 2-18-485 | N-tolyl | N-tolyl | H | b3 | b3 |
| 2-18-486 | N-tolyl | N-tolyl | t-bu | b3 | b3 |
| 2-18-487 | N-tolyl | N-tolyl | methyl | b3 | b3 |
| 2-18-488 | N-tolyl | N-tolyl | b1 | b3 | b3 |
| 2-18-489 | N-tolyl | N-tolyl | b2 | b3 | b3 |
| 2-18-490 | N-tolyl | N-tolyl | b3 | b3 | b3 |
| 2-18-491 | N-tolyl | N-tolyl | H | b5 | b5 |
| 2-18-492 | N-tolyl | N-tolyl | t-bu | b5 | b5 |
| 2-18-493 | N-tolyl | N-tolyl | methyl | b5 | b5 |
| 2-18-494 | N-tolyl | N-tolyl | b1 | b5 | b5 |
| 2-18-495 | N-tolyl | N-tolyl | b2 | b5 | b5 |
| 2-18-496 | N-tolyl | N-tolyl | b3 | b5 | b5 |
| 2-18-497 | N-tolyl | N-tolyl | H | b6 | b6 |
| 2-18-498 | N-tolyl | N-tolyl | t-bu | b6 | b6 |
| 2-18-499 | N-tolyl | N-tolyl | methyl | b6 | b6 |
| 2-18-500 | N-tolyl | N-tolyl | b1 | b6 | b6 |
| 2-18-501 | N-tolyl | N-tolyl | b2 | b6 | b6 |
| 2-18-502 | N-tolyl | N-tolyl | b3 | b6 | b6 |
| 2-18-503 | N-tolyl | N-tolyl | H | b7 | b7 |
| 2-18-504 | N-tolyl | N-tolyl | t-bu | b7 | b7 |
| 2-18-505 | N-tolyl | N-tolyl | methyl | b7 | b7 |
| 2-18-506 | N-tolyl | N-tolyl | b1 | b7 | b7 |
| 2-18-507 | N-tolyl | N-tolyl | b2 | b7 | b7 |
| 2-18-508 | N-tolyl | N-tolyl | b3 | b7 | b7 |
| 2-18-509 | N-Biph | N-Biph | H | H | H |
| 2-18-510 | N-Biph | N-Biph | t-bu | H | H |
| 2-18-511 | N-Biph | N-Biph | methyl | H | H |
| 2-18-512 | N-Biph | N-Biph | t-bu | t-bu | t-bu |
| 2-18-513 | N-Biph | N-Biph | methyl | methyl | methyl |
| 2-18-514 | N-Biph | N-Biph | b1 | H | H |
| 2-18-515 | N-Biph | N-Biph | b2 | H | H |
| 2-18-516 | N-Biph | N-Biph | b3 | H | H |
| 2-18-517 | N-Biph | N-Biph | H | H | a1 |
| 2-18-518 | N-Biph | N-Biph | t-bu | H | a1 |
| 2-18-519 | N-Biph | N-Biph | methyl | H | a1 |
| 2-18-520 | N-Biph | N-Biph | b1 | H | a1 |
| 2-18-521 | N-Biph | N-Biph | b2 | H | a1 |
| 2-18-522 | N-Biph | N-Biph | b3 | H | a1 |
| 2-18-523 | N-Biph | N-Biph | H | H | a2 |
| 2-18-524 | N-Biph | N-Biph | t-bu | H | a2 |
| 2-18-525 | N-Biph | N-Biph | methyl | H | a2 |
| 2-18-526 | N-Biph | N-Biph | b1 | H | a2 |
| 2-18-527 | N-Biph | N-Biph | b2 | H | a2 |
| 2-18-528 | N-Biph | N-Biph | b3 | H | a2 |
| 2-18-529 | N-Biph | N-Biph | H | H | a3 |
| 2-18-530 | N-Biph | N-Biph | t-bu | H | a3 |
| 2-18-531 | N-Biph | N-Biph | methyl | H | a3 |
| 2-18-532 | N-Biph | N-Biph | b1 | H | a3 |
| 2-18-533 | N-Biph | N-Biph | b2 | H | a3 |
| 2-18-534 | N-Biph | N-Biph | b3 | H | a3 |
| 2-18-535 | N-Biph | N-Biph | H | H | a4 |
| 2-18-536 | N-Biph | N-Biph | t-bu | H | a4 |
| 2-18-537 | N-Biph | N-Biph | methyl | H | a4 |
| 2-18-538 | N-Biph | N-Biph | b1 | H | a4 |
| 2-18-539 | N-Biph | N-Biph | b2 | H | a4 |
| 2-18-540 | N-Biph | N-Biph | b3 | H | a4 |
| 2-18-541 | N-Biph | N-Biph | H | H | a5 |
| 2-18-542 | N-Biph | N-Biph | t-bu | H | a5 |
| 2-18-543 | N-Biph | N-Biph | methyl | H | a5 |
| 2-18-544 | N-Biph | N-Biph | b1 | H | a5 |
| 2-18-545 | N-Biph | N-Biph | b2 | H | a5 |
| 2-18-546 | N-Biph | N-Biph | b3 | H | a5 |
| 2-18-547 | N-Biph | N-Biph | H | H | a6 |
| 2-18-548 | N-Biph | N-Biph | t-bu | H | a6 |
| 2-18-549 | N-Biph | N-Biph | methyl | H | a6 |
| 2-18-550 | N-Biph | N-Biph | b1 | H | a6 |
| 2-18-551 | N-Biph | N-Biph | b2 | H | a6 |
| 2-18-552 | N-Biph | N-Biph | b3 | H | a6 |
| 2-18-553 | N-Biph | N-Biph | H | H | b1 |
| 2-18-554 | N-Biph | N-Biph | t-bu | H | b1 |
| 2-18-555 | N-Biph | N-Biph | methyl | H | b1 |
| 2-18-556 | N-Biph | N-Biph | b1 | H | b1 |
| 2-18-557 | N-Biph | N-Biph | b2 | H | b1 |
| 2-18-558 | N-Biph | N-Biph | b3 | H | b1 |
| 2-18-559 | N-Biph | N-Biph | H | H | b2 |
| 2-18-560 | N-Biph | N-Biph | t-bu | H | b2 |
| 2-18-561 | N-Biph | N-Biph | methyl | H | b2 |
| 2-18-562 | N-Biph | N-Biph | b1 | H | b2 |
| 2-18-563 | N-Biph | N-Biph | b2 | H | b2 |
| 2-18-564 | N-Biph | N-Biph | b3 | H | b2 |
| 2-18-565 | N-Biph | N-Biph | H | H | b3 |
| 2-18-566 | N-Biph | N-Biph | t-bu | H | b3 |
| 2-18-567 | N-Biph | N-Biph | methyl | H | b3 |
| 2-18-568 | N-Biph | N-Biph | b1 | H | b3 |
| 2-18-569 | N-Biph | N-Biph | b2 | H | b3 |
| 2-18-570 | N-Biph | N-Biph | b3 | H | b3 |
| 2-18-571 | N-Biph | N-Biph | H | H | b4 |
| 2-18-572 | N-Biph | N-Biph | t-bu | H | b4 |
| 2-18-573 | N-Biph | N-Biph | methyl | H | b4 |
| 2-18-574 | N-Biph | N-Biph | b1 | H | b4 |
| 2-18-575 | N-Biph | N-Biph | b2 | H | b4 |
| 2-18-576 | N-Biph | N-Biph | b3 | H | b4 |
| 2-18-577 | N-Biph | N-Biph | H | H | b5 |
| 2-18-578 | N-Biph | N-Biph | t-bu | H | b5 |
| 2-18-579 | N-Biph | N-Biph | methyl | H | b5 |
| 2-18-580 | N-Biph | N-Biph | b1 | H | b5 |
| 2-18-581 | N-Biph | N-Biph | b2 | H | b5 |
| 2-18-582 | N-Biph | N-Biph | b3 | H | b5 |
| 2-18-583 | N-Biph | N-Biph | H | H | b6 |
| 2-18-584 | N-Biph | N-Biph | t-bu | H | b6 |
| 2-18-585 | N-Biph | N-Biph | methyl | H | b6 |
| 2-18-586 | N-Biph | N-Biph | b1 | H | b6 |
| 2-18-587 | N-Biph | N-Biph | b2 | H | b6 |
| 2-18-588 | N-Biph | N-Biph | b3 | H | b6 |
| 2-18-589 | N-Biph | N-Biph | H | H | b7 |
| 2-18-590 | N-Biph | N-Biph | t-bu | H | b7 |
| 2-18-591 | N-Biph | N-Biph | methyl | H | b7 |
| 2-18-592 | N-Biph | N-Biph | b1 | H | b7 |
| 2-18-593 | N-Biph | N-Biph | b2 | H | b7 |
| 2-18-594 | N-Biph | N-Biph | b3 | H | b7 |
| 2-18-595 | N-Biph | N-Biph | H | H | b8 |
| 2-18-596 | N-Biph | N-Biph | t-bu | H | b8 |
| 2-18-597 | N-Biph | N-Biph | methyl | H | b8 |
| 2-18-598 | N-Biph | N-Biph | b1 | H | b8 |
| 2-18-599 | N-Biph | N-Biph | b2 | H | b8 |
| 2-18-600 | N-Biph | N-Biph | b3 | H | b8 |
| 2-18-601 | N-Biph | N-Biph | H | H | b9 |
| 2-18-602 | N-Biph | N-Biph | t-bu | H | b9 |
| 2-18-603 | N-Biph | N-Biph | methyl | H | b9 |
| 2-18-604 | N-Biph | N-Biph | b1 | H | b9 |
| 2-18-605 | N-Biph | N-Biph | b2 | H | b9 |
| 2-18-606 | N-Biph | N-Biph | b3 | H | b9 |
| 2-18-607 | N-Biph | N-Biph | H | H | b10 |
| 2-18-608 | N-Biph | N-Biph | t-bu | H | b10 |
| 2-18-609 | N-Biph | N-Biph | methyl | H | b10 |
| 2-18-610 | N-Biph | N-Biph | b1 | H | b10 |
| 2-18-611 | N-Biph | N-Biph | b2 | H | b10 |
| 2-18-612 | N-Biph | N-Biph | b3 | H | b10 |
| 2-18-613 | N-Biph | N-Biph | H | a1 | H |
| 2-18-614 | N-Biph | N-Biph | t-bu | a1 | H |
| 2-18-615 | N-Biph | N-Biph | methyl | a1 | H |
| 2-18-616 | N-Biph | N-Biph | b1 | a1 | H |
| 2-18-617 | N-Biph | N-Biph | b2 | a1 | H |
| 2-18-618 | N-Biph | N-Biph | b3 | a1 | H |
| 2-18-619 | N-Biph | N-Biph | H | a2 | H |
| 2-18-620 | N-Biph | N-Biph | t-bu | a2 | H |
| 2-18-621 | N-Biph | N-Biph | methyl | a2 | H |
| 2-18-622 | N-Biph | N-Biph | b1 | a2 | H |
| 2-18-623 | N-Biph | N-Biph | b2 | a2 | H |
| 2-18-624 | N-Biph | N-Biph | b3 | a2 | H |
| 2-18-625 | N-Biph | N-Biph | H | a3 | H |
| 2-18-626 | N-Biph | N-Biph | t-bu | a3 | H |
| 2-18-627 | N-Biph | N-Biph | methyl | a3 | H |
| 2-18-628 | N-Biph | N-Biph | b1 | a3 | H |
| 2-18-629 | N-Biph | N-Biph | b2 | a3 | H |
| 2-18-630 | N-Biph | N-Biph | b3 | a3 | H |
| 2-18-631 | N-Biph | N-Biph | H | a4 | H |
| 2-18-632 | N-Biph | N-Biph | t-bu | a4 | H |
| 2-18-633 | N-Biph | N-Biph | methyl | a4 | H |
| 2-18-634 | N-Biph | N-Biph | b1 | a4 | H |
| 2-18-635 | N-Biph | N-Biph | b2 | a4 | H |
| 2-18-636 | N-Biph | N-Biph | b3 | a4 | H |
| 2-18-637 | N-Biph | N-Biph | H | a5 | H |
| 2-18-638 | N-Biph | N-Biph | t-bu | a5 | H |
| 2-18-639 | N-Biph | N-Biph | methyl | a5 | H |
| 2-18-640 | N-Biph | N-Biph | b1 | a5 | H |
| 2-18-641 | N-Biph | N-Biph | b2 | a5 | H |
| 2-18-642 | N-Biph | N-Biph | b3 | a5 | H |
| 2-18-643 | N-Biph | N-Biph | H | a6 | H |
| 2-18-644 | N-Biph | N-Biph | t-bu | a6 | H |
| 2-18-645 | N-Biph | N-Biph | methyl | a6 | H |
| 2-18-646 | N-Biph | N-Biph | b1 | a6 | H |
| 2-18-647 | N-Biph | N-Biph | b2 | a6 | H |
| 2-18-648 | N-Biph | N-Biph | b3 | a6 | H |
| 2-18-649 | N-Biph | N-Biph | H | b1 | H |
| 2-18-650 | N-Biph | N-Biph | t-bu | b1 | H |
| 2-18-651 | N-Biph | N-Biph | methyl | b1 | H |
| 2-18-652 | N-Biph | N-Biph | b1 | b1 | H |
| 2-18-653 | N-Biph | N-Biph | b2 | b1 | H |
| 2-18-654 | N-Biph | N-Biph | b3 | b1 | H |
| 2-18-655 | N-Biph | N-Biph | H | b2 | H |
| 2-18-656 | N-Biph | N-Biph | t-bu | b2 | H |
| 2-18-657 | N-Biph | N-Biph | methyl | b2 | H |
| 2-18-658 | N-Biph | N-Biph | b1 | b2 | H |
| 2-18-659 | N-Biph | N-Biph | b2 | b2 | H |
| 2-18-660 | N-Biph | N-Biph | b3 | b2 | H |
| 2-18-661 | N-Biph | N-Biph | H | b3 | H |
| 2-18-662 | N-Biph | N-Biph | t-bu | b3 | H |
| 2-18-663 | N-Biph | N-Biph | methyl | b3 | H |
| 2-18-664 | N-Biph | N-Biph | b1 | b3 | H |
| 2-18-665 | N-Biph | N-Biph | b2 | b3 | H |
| 2-18-666 | N-Biph | N-Biph | b3 | b3 | H |
| 2-18-667 | N-Biph | N-Biph | H | b4 | H |
| 2-18-668 | N-Biph | N-Biph | t-bu | b4 | H |
| 2-18-669 | N-Biph | N-Biph | methyl | b4 | H |
| 2-18-670 | N-Biph | N-Biph | b1 | b4 | H |
| 2-18-671 | N-Biph | N-Biph | b2 | b4 | H |
| 2-18-672 | N-Biph | N-Biph | b3 | b4 | H |
| 2-18-673 | N-Biph | N-Biph | H | b5 | H |
| 2-18-674 | N-Biph | N-Biph | t-bu | b5 | H |
| 2-18-675 | N-Biph | N-Biph | methyl | b5 | H |
| 2-18-676 | N-Biph | N-Biph | b1 | b5 | H |
| 2-18-677 | N-Biph | N-Biph | b2 | b5 | H |
| 2-18-678 | N-Biph | N-Biph | b3 | b5 | H |
| 2-18-679 | N-Biph | N-Biph | H | b6 | H |
| 2-18-680 | N-Biph | N-Biph | t-bu | b6 | H |
| 2-18-681 | N-Biph | N-Biph | methyl | b6 | H |
| 2-18-682 | N-Biph | N-Biph | b1 | b6 | H |
| 2-18-683 | N-Biph | N-Biph | b2 | b6 | H |
| 2-18-684 | N-Biph | N-Biph | b3 | b6 | H |
| 2-18-685 | N-Biph | N-Biph | H | b7 | H |
| 2-18-686 | N-Biph | N-Biph | t-bu | b7 | H |
| 2-18-687 | N-Biph | N-Biph | methyl | b7 | H |
| 2-18-688 | N-Biph | N-Biph | b1 | b7 | H |
| 2-18-689 | N-Biph | N-Biph | b2 | b7 | H |
| 2-18-690 | N-Biph | N-Biph | b3 | b7 | H |
| 2-18-691 | N-Biph | N-Biph | H | b8 | H |
| 2-18-692 | N-Biph | N-Biph | t-bu | b8 | H |
| 2-18-693 | N-Biph | N-Biph | methyl | b8 | H |
| 2-18-694 | N-Biph | N-Biph | b1 | b8 | H |
| 2-18-695 | N-Biph | N-Biph | b2 | b8 | H |
| 2-18-696 | N-Biph | N-Biph | b3 | b8 | H |
| 2-18-697 | N-Biph | N-Biph | H | b9 | H |
| 2-18-698 | N-Biph | N-Biph | t-bu | b9 | H |
| 2-18-699 | N-Biph | N-Biph | methyl | b9 | H |
| 2-18-700 | N-Biph | N-Biph | b1 | b9 | H |
| 2-18-701 | N-Biph | N-Biph | b2 | b9 | H |
| 2-18-702 | N-Biph | N-Biph | b3 | b9 | H |
| 2-18-703 | N-Biph | N-Biph | H | b10 | H |
| 2-18-704 | N-Biph | N-Biph | t-bu | b10 | H |
| 2-18-705 | N-Biph | N-Biph | methyl | b10 | H |
| 2-18-706 | N-Biph | N-Biph | b1 | b10 | H |
| 2-18-707 | N-Biph | N-Biph | b2 | b10 | H |
| 2-18-708 | N-Biph | N-Biph | b3 | b10 | H |
| 2-18-709 | N-Biph | N-Biph | H | a1 | a1 |
| 2-18-710 | N-Biph | N-Biph | t-bu | a1 | a1 |
| 2-18-711 | N-Biph | N-Biph | methyl | a1 | a1 |
| 2-18-712 | N-Biph | N-Biph | b1 | a1 | a1 |
| 2-18-713 | N-Biph | N-Biph | b2 | a1 | a1 |
| 2-18-714 | N-Biph | N-Biph | b3 | a1 | a1 |
| 2-18-715 | N-Biph | N-Biph | H | a2 | a2 |
| 2-18-716 | N-Biph | N-Biph | t-bu | a2 | a2 |
| 2-18-717 | N-Biph | N-Biph | methyl | a2 | a2 |
| 2-18-718 | N-Biph | N-Biph | b1 | a2 | a2 |
| 2-18-719 | N-Biph | N-Biph | b2 | a2 | a2 |
| 2-18-720 | N-Biph | N-Biph | b3 | a2 | a2 |
| 2-18-721 | N-Biph | N-Biph | H | a3 | a3 |
| 2-18-722 | N-Biph | N-Biph | t-bu | a3 | a3 |
| 2-18-723 | N-Biph | N-Biph | methyl | a3 | a3 |
| 2-18-724 | N-Biph | N-Biph | b1 | a3 | a3 |
| 2-18-725 | N-Biph | N-Biph | b2 | a3 | a3 |
| 2-18-726 | N-Biph | N-Biph | b3 | a3 | a3 |
| 2-18-727 | N-Biph | N-Biph | H | b1 | b1 |
| 2-18-728 | N-Biph | N-Biph | t-bu | b1 | b1 |
| 2-18-729 | N-Biph | N-Biph | methyl | b1 | b1 |
| 2-18-730 | N-Biph | N-Biph | b1 | b1 | b1 |
| 2-18-731 | N-Biph | N-Biph | b2 | b1 | b1 |
| 2-18-732 | N-Biph | N-Biph | b3 | b1 | b1 |
| 2-18-733 | N-Biph | N-Biph | H | b2 | b2 |
| 2-18-734 | N-Biph | N-Biph | t-bu | b2 | b2 |
| 2-18-735 | N-Biph | N-Biph | methyl | b2 | b2 |
| 2-18-736 | N-Biph | N-Biph | b1 | b2 | b2 |
| 2-18-737 | N-Biph | N-Biph | b2 | b2 | b2 |
| 2-18-738 | N-Biph | N-Biph | b3 | b2 | b2 |
| 2-18-739 | N-Biph | N-Biph | H | b3 | b3 |
| 2-18-740 | N-Biph | N-Biph | t-bu | b3 | b3 |
| 2-18-741 | N-Biph | N-Biph | methyl | b3 | b3 |
| 2-18-742 | N-Biph | N-Biph | b1 | b3 | b3 |
| 2-18-743 | N-Biph | N-Biph | b2 | b3 | b3 |
| 2-18-744 | N-Biph | N-Biph | b3 | b3 | b3 |
| 2-18-745 | N-Biph | N-Biph | H | b5 | b5 |
| 2-18-746 | N-Biph | N-Biph | t-bu | b5 | b5 |
| 2-18-747 | N-Biph | N-Biph | methyl | b5 | b5 |
| 2-18-748 | N-Biph | N-Biph | b1 | b5 | b5 |
| 2-18-749 | N-Biph | N-Biph | b2 | b5 | b5 |
| 2-18-750 | N-Biph | N-Biph | b3 | b5 | b5 |
| 2-18-751 | N-Biph | N-Biph | H | b6 | b6 |
| 2-18-752 | N-Biph | N-Biph | t-bu | b6 | b6 |
| 2-18-753 | N-Biph | N-Biph | methyl | b6 | b6 |
| 2-18-754 | N-Biph | N-Biph | b1 | b6 | b6 |
| 2-18-755 | N-Biph | N-Biph | b2 | b6 | b6 |
| 2-18-756 | N-Biph | N-Biph | b3 | b6 | b6 |
| 2-18-757 | N-Biph | N-Biph | H | b7 | b7 |
| 2-18-758 | N-Biph | N-Biph | t-bu | b7 | b7 |
| 2-18-759 | N-Biph | N-Biph | methyl | b7 | b7 |
| 2-18-760 | N-Biph | N-Biph | b1 | b7 | b7 |
| 2-18-761 | N-Biph | N-Biph | b2 | b7 | b7 |
| 2-18-762 | N-Biph | N-Biph | b3 | b7 | b7 |
| 2-18-763 | O | N—Ph | H | H | H |
| 2-18-764 | O | N—Ph | t-bu | H | H |
| 2-18-765 | O | N—Ph | methyl | H | H |
| 2-18-766 | O | N—Ph | t-bu | t-bu | t-bu |
| 2-18-767 | O | N—Ph | methyl | methyl | methyl |
| 2-18-768 | O | N—Ph | b1 | H | H |
| 2-18-769 | O | N—Ph | b2 | H | H |
| 2-18-770 | O | N—Ph | b3 | H | H |
| 2-18-771 | O | N—Ph | H | H | a1 |
| 2-18-772 | O | N—Ph | t-bu | H | a1 |
| 2-18-773 | O | N—Ph | methyl | H | a1 |
| 2-18-774 | O | N—Ph | b1 | H | a1 |
| 2-18-775 | O | N—Ph | b2 | H | a1 |
| 2-18-776 | O | N—Ph | b3 | H | a1 |
| 2-18-777 | O | N—Ph | H | H | a2 |
| 2-18-778 | O | N—Ph | t-bu | H | a2 |
| 2-18-779 | O | N—Ph | methyl | H | a2 |
| 2-18-780 | O | N—Ph | b1 | H | a2 |
| 2-18-781 | O | N—Ph | b2 | H | a2 |
| 2-18-782 | O | N—Ph | b3 | H | a2 |
| 2-18-783 | O | N—Ph | H | H | a3 |
| 2-18-784 | O | N—Ph | t-bu | H | a3 |
| 2-18-785 | O | N—Ph | methyl | H | a3 |
| 2-18-786 | O | N—Ph | b1 | H | a3 |
| 2-18-787 | O | N—Ph | b2 | H | a3 |
| 2-18-788 | O | N—Ph | b3 | H | a3 |
| 2-18-789 | O | N—Ph | H | H | a4 |
| 2-18-790 | O | N—Ph | t-bu | H | a4 |
| 2-18-791 | O | N—Ph | methyl | H | a4 |
| 2-18-792 | O | N—Ph | b1 | H | a4 |
| 2-18-793 | O | N—Ph | b2 | H | a4 |
| 2-18-794 | O | N—Ph | b3 | H | a4 |
| 2-18-795 | O | N—Ph | H | H | a5 |
| 2-18-796 | O | N—Ph | t-bu | H | a5 |
| 2-18-797 | O | N—Ph | methyl | H | a5 |
| 2-18-798 | O | N—Ph | b1 | H | a5 |
| 2-18-799 | O | N—Ph | b2 | H | a5 |
| 2-18-800 | O | N—Ph | b3 | H | a5 |
| 2-18-801 | O | N—Ph | H | H | a6 |
| 2-18-802 | O | N—Ph | t-bu | H | a6 |
| 2-18-803 | O | N—Ph | methyl | H | a6 |
| 2-18-804 | O | N—Ph | b1 | H | a6 |
| 2-18-805 | O | N—Ph | b2 | H | a6 |
| 2-18-806 | O | N—Ph | b3 | H | a6 |
| 2-18-807 | O | N—Ph | H | H | b1 |
| 2-18-808 | O | N—Ph | t-bu | H | b1 |
| 2-18-809 | O | N—Ph | methyl | H | b1 |
| 2-18-810 | O | N—Ph | b1 | H | b1 |
| 2-18-811 | O | N—Ph | b2 | H | b1 |
| 2-18-812 | O | N—Ph | b3 | H | b1 |
| 2-18-813 | O | N—Ph | H | H | b2 |
| 2-18-814 | O | N—Ph | t-bu | H | b2 |
| 2-18-815 | O | N—Ph | methyl | H | b2 |
| 2-18-816 | O | N—Ph | b1 | H | b2 |
| 2-18-817 | O | N—Ph | b2 | H | b2 |
| 2-18-818 | O | N—Ph | b3 | H | b2 |
| 2-18-819 | O | N—Ph | H | H | b3 |
| 2-18-820 | O | N—Ph | t-bu | H | b3 |
| 2-18-821 | O | N—Ph | methyl | H | b3 |
| 2-18-822 | O | N—Ph | b1 | H | b3 |
| 2-18-823 | O | N—Ph | b2 | H | b3 |
| 2-18-824 | O | N—Ph | b3 | H | b3 |
| 2-18-825 | O | N—Ph | H | H | b4 |
| 2-18-826 | O | N—Ph | t-bu | H | b4 |
| 2-18-827 | O | N—Ph | methyl | H | b4 |
| 2-18-828 | O | N—Ph | b1 | H | b4 |
| 2-18-829 | O | N—Ph | b2 | H | b4 |
| 2-18-830 | O | N—Ph | b3 | H | b4 |
| 2-18-831 | O | N—Ph | H | H | b5 |
| 2-18-832 | O | N—Ph | t-bu | H | b5 |
| 2-18-833 | O | N—Ph | methyl | H | b5 |
| 2-18-834 | O | N—Ph | b1 | H | b5 |
| 2-18-835 | O | N—Ph | b2 | H | b5 |
| 2-18-836 | O | N—Ph | b3 | H | b5 |
| 2-18-837 | O | N—Ph | H | H | b6 |
| 2-18-838 | O | N—Ph | t-bu | H | b6 |
| 2-18-839 | O | N—Ph | methyl | H | b6 |
| 2-18-840 | O | N—Ph | b1 | H | b6 |
| 2-18-841 | O | N—Ph | b2 | H | b6 |
| 2-18-842 | O | N—Ph | b3 | H | b6 |
| 2-18-843 | O | N—Ph | H | H | b7 |
| 2-18-844 | O | N—Ph | t-bu | H | b7 |
| 2-18-845 | O | N—Ph | methyl | H | b7 |
| 2-18-846 | O | N—Ph | b1 | H | b7 |
| 2-18-847 | O | N—Ph | b2 | H | b7 |
| 2-18-848 | O | N—Ph | b3 | H | b7 |
| 2-18-849 | O | N—Ph | H | H | b8 |
| 2-18-850 | O | N—Ph | t-bu | H | b8 |
| 2-18-851 | O | N—Ph | methyl | H | b8 |
| 2-18-852 | O | N—Ph | b1 | H | b8 |
| 2-18-853 | O | N—Ph | b2 | H | b8 |
| 2-18-854 | O | N—Ph | b3 | H | b8 |
| 2-18-855 | O | N—Ph | H | H | b9 |
| 2-18-856 | O | N—Ph | t-bu | H | b9 |
| 2-18-857 | O | N—Ph | methyl | H | b9 |
| 2-18-858 | O | N—Ph | b1 | H | b9 |
| 2-18-859 | O | N—Ph | b2 | H | b9 |
| 2-18-860 | O | N—Ph | b3 | H | b9 |
| 2-18-861 | O | N—Ph | H | H | b10 |
| 2-18-862 | O | N—Ph | t-bu | H | b10 |
| 2-18-863 | O | N—Ph | methyl | H | b10 |
| 2-18-864 | O | N—Ph | b1 | H | b10 |
| 2-18-865 | O | N—Ph | b2 | H | b10 |
| 2-18-866 | O | N—Ph | b3 | H | b10 |
| 2-18-867 | O | N—Ph | H | a1 | H |
| 2-18-868 | O | N—Ph | t-bu | a1 | H |
| 2-18-869 | O | N—Ph | methyl | a1 | H |
| 2-18-870 | O | N—Ph | b1 | a1 | H |
| 2-18-871 | O | N—Ph | b2 | a1 | H |
| 2-18-872 | O | N—Ph | b3 | a1 | H |
| 2-18-873 | O | N—Ph | H | a2 | H |
| 2-18-874 | O | N—Ph | t-bu | a2 | H |
| 2-18-875 | O | N—Ph | methyl | a2 | H |
| 2-18-876 | O | N—Ph | b1 | a2 | H |
| 2-18-877 | O | N—Ph | b2 | a2 | H |
| 2-18-878 | O | N—Ph | b3 | a2 | H |
| 2-18-879 | O | N—Ph | H | a3 | H |
| 2-18-880 | O | N—Ph | t-bu | a3 | H |
| 2-18-881 | O | N—Ph | methyl | a3 | H |
| 2-18-882 | O | N—Ph | b1 | a3 | H |
| 2-18-883 | O | N—Ph | b2 | a3 | H |
| 2-18-884 | O | N—Ph | b3 | a3 | H |
| 2-18-885 | O | N—Ph | H | a4 | H |
| 2-18-886 | O | N—Ph | t-bu | a4 | H |
| 2-18-887 | O | N—Ph | methyl | a4 | H |
| 2-18-888 | O | N—Ph | b1 | a4 | H |
| 2-18-889 | O | N—Ph | b2 | a4 | H |
| 2-18-890 | O | N—Ph | b3 | a4 | H |
| 2-18-891 | O | N—Ph | H | a5 | H |
| 2-18-892 | O | N—Ph | t-bu | a5 | H |
| 2-18-893 | O | N—Ph | methyl | a5 | H |
| 2-18-894 | O | N—Ph | b1 | a5 | H |
| 2-18-895 | O | N—Ph | b2 | a5 | H |
| 2-18-896 | O | N—Ph | b3 | a5 | H |
| 2-18-897 | O | N—Ph | H | a6 | H |
| 2-18-898 | O | N—Ph | t-bu | a6 | H |
| 2-18-899 | O | N—Ph | methyl | a6 | H |
| 2-18-900 | O | N—Ph | b1 | a6 | H |
| 2-18-901 | O | N—Ph | b2 | a6 | H |
| 2-18-902 | O | N—Ph | b3 | a6 | H |
| 2-18-903 | O | N—Ph | H | b1 | H |
| 2-18-904 | O | N—Ph | t-bu | b1 | H |
| 2-18-905 | O | N—Ph | methyl | b1 | H |
| 2-18-906 | O | N—Ph | b1 | b1 | H |
| 2-18-907 | O | N—Ph | b2 | b1 | H |
| 2-18-908 | O | N—Ph | b3 | b1 | H |
| 2-18-909 | O | N—Ph | H | b2 | H |
| 2-18-910 | O | N—Ph | t-bu | b2 | H |
| 2-18-911 | O | N—Ph | methyl | b2 | H |
| 2-18-912 | O | N—Ph | b1 | b2 | H |
| 2-18-913 | O | N—Ph | b2 | b2 | H |
| 2-18-914 | O | N—Ph | b3 | b2 | H |
| 2-18-915 | O | N—Ph | H | b3 | H |
| 2-18-916 | O | N—Ph | t-bu | b3 | H |
| 2-18-917 | O | N—Ph | methyl | b3 | H |
| 2-18-918 | O | N—Ph | b1 | b3 | H |
| 2-18-919 | O | N—Ph | b2 | b3 | H |
| 2-18-920 | O | N—Ph | b3 | b3 | H |
| 2-18-921 | O | N—Ph | H | b4 | H |
| 2-18-922 | O | N—Ph | t-bu | b4 | H |
| 2-18-923 | O | N—Ph | methyl | b4 | H |
| 2-18-924 | O | N—Ph | b1 | b4 | H |
| 2-18-925 | O | N—Ph | b2 | b4 | H |
| 2-18-926 | O | N—Ph | b3 | b4 | H |
| 2-18-927 | O | N—Ph | H | b5 | H |
| 2-18-928 | O | N—Ph | t-bu | b5 | H |
| 2-18-929 | O | N—Ph | methyl | b5 | H |
| 2-18-930 | O | N—Ph | b1 | b5 | H |
| 2-18-931 | O | N—Ph | b2 | b5 | H |
| 2-18-932 | O | N—Ph | b3 | b5 | H |
| 2-18-933 | O | N—Ph | H | b6 | H |
| 2-18-934 | O | N—Ph | t-bu | b6 | H |
| 2-18-935 | O | N—Ph | methyl | b6 | H |
| 2-18-936 | O | N—Ph | b1 | b6 | H |
| 2-18-937 | O | N—Ph | b2 | b6 | H |
| 2-18-938 | O | N—Ph | b3 | b6 | H |
| 2-18-939 | O | N—Ph | H | b7 | H |
| 2-18-940 | O | N—Ph | t-bu | b7 | H |
| 2-18-941 | O | N—Ph | methyl | b7 | H |
| 2-18-942 | O | N—Ph | b1 | b7 | H |
| 2-18-943 | O | N—Ph | b2 | b7 | H |
| 2-18-944 | O | N—Ph | b3 | b7 | H |
| 2-18-945 | O | N—Ph | H | b8 | H |
| 2-18-946 | O | N—Ph | t-bu | b8 | H |
| 2-18-947 | O | N—Ph | methyl | b8 | H |
| 2-18-948 | O | N—Ph | b1 | b8 | H |
| 2-18-949 | O | N—Ph | b2 | b8 | H |
| 2-18-950 | O | N—Ph | b3 | b8 | H |
| 2-18-951 | O | N—Ph | H | b9 | H |
| 2-18-952 | O | N—Ph | t-bu | b9 | H |
| 2-18-953 | O | N—Ph | methyl | b9 | H |
| 2-18-954 | O | N—Ph | b1 | b9 | H |
| 2-18-955 | O | N—Ph | b2 | b9 | H |
| 2-18-956 | O | N—Ph | b3 | b9 | H |
| 2-18-957 | O | N—Ph | H | b10 | H |
| 2-18-958 | O | N—Ph | t-bu | b10 | H |
| 2-18-959 | O | N—Ph | methyl | b10 | H |
| 2-18-960 | O | N—Ph | b1 | b10 | H |
| 2-18-961 | O | N—Ph | b2 | b10 | H |
| 2-18-962 | O | N—Ph | b3 | b10 | H |
| 2-18-963 | O | N—Ph | H | a1 | a1 |
| 2-18-964 | O | N—Ph | t-bu | a1 | a1 |
| 2-18-965 | O | N—Ph | methyl | a1 | a1 |
| 2-18-966 | O | N—Ph | b1 | a1 | a1 |
| 2-18-967 | O | N—Ph | b2 | a1 | a1 |
| 2-18-968 | O | N—Ph | b3 | a1 | a1 |
| 2-18-969 | O | N—Ph | H | a2 | a2 |
| 2-18-970 | O | N—Ph | t-bu | a2 | a2 |
| 2-18-971 | O | N—Ph | methyl | a2 | a2 |
| 2-18-972 | O | N—Ph | b1 | a2 | a2 |
| 2-18-973 | O | N—Ph | b2 | a2 | a2 |
| 2-18-974 | O | N—Ph | b3 | a2 | a2 |
| 2-18-975 | O | N—Ph | H | a3 | a3 |
| 2-18-976 | O | N—Ph | t-bu | a3 | a3 |
| 2-18-977 | O | N—Ph | methyl | a3 | a3 |
| 2-18-978 | O | N—Ph | b1 | a3 | a3 |
| 2-18-979 | O | N—Ph | b2 | a3 | a3 |
| 2-18-980 | O | N—Ph | b3 | a3 | a3 |
| 2-18-981 | O | N—Ph | H | b1 | b1 |
| 2-18-982 | O | N—Ph | t-bu | b1 | b1 |
| 2-18-983 | O | N—Ph | methyl | b1 | b1 |
| 2-18-984 | O | N—Ph | b1 | b1 | b1 |
| 2-18-985 | O | N—Ph | b2 | b1 | b1 |
| 2-18-986 | O | N—Ph | b3 | b1 | b1 |
| 2-18-987 | O | N—Ph | H | b2 | b2 |
| 2-18-988 | O | N—Ph | t-bu | b2 | b2 |
| 2-18-989 | O | N—Ph | methyl | b2 | b2 |
| 2-18-990 | O | N—Ph | b1 | b2 | b2 |
| 2-18-991 | O | N—Ph | b2 | b2 | b2 |
| 2-18-992 | O | N—Ph | b3 | b2 | b2 |
| 2-18-993 | O | N—Ph | H | b3 | b3 |
| 2-18-994 | O | N—Ph | t-bu | b3 | b3 |
| 2-18-995 | O | N—Ph | methyl | b3 | b3 |
| 2-18-996 | O | N—Ph | b1 | b3 | b3 |
| 2-18-997 | O | N—Ph | b2 | b3 | b3 |
| 2-18-998 | O | N—Ph | b3 | b3 | b3 |
| 2-18-999 | O | N—Ph | H | b5 | b5 |
| 2-18-1000 | O | N—Ph | t-bu | b5 | b5 |
| 2-18-1001 | O | N—Ph | methyl | b5 | b5 |
| 2-18-1002 | O | N—Ph | b1 | b5 | b5 |
| 2-18-1003 | O | N—Ph | b2 | b5 | b5 |
| 2-18-1004 | O | N—Ph | b3 | b5 | b5 |
| 2-18-1005 | O | N—Ph | H | b6 | b6 |
| 2-18-1006 | O | N—Ph | t-bu | b6 | b6 |
| 2-18-1007 | O | N—Ph | methyl | b6 | b6 |
| 2-18-1008 | O | N—Ph | b1 | b6 | b6 |
| 2-18-1009 | O | N—Ph | b2 | b6 | b6 |
| 2-18-1010 | O | N—Ph | b3 | b6 | b6 |
| 2-18-1011 | O | N—Ph | H | b7 | b7 |
| 2-18-1012 | O | N—Ph | t-bu | b7 | b7 |
| 2-18-1013 | O | N—Ph | methyl | b7 | b7 |
| 2-18-1014 | O | N—Ph | b1 | b7 | b7 |
| 2-18-1015 | O | N—Ph | b2 | b7 | b7 |
| 2-18-1016 | O | N—Ph | b3 | b7 | b7 |
| 2-18-1017 | S | N—Ph | H | H | H |
| 2-18-1018 | S | N—Ph | t-bu | H | H |
| 2-18-1019 | S | N—Ph | methyl | H | H |
| 2-18-1020 | S | N—Ph | t-bu | t-bu | t-bu |
| 2-18-1021 | S | N—Ph | methyl | methyl | methyl |
| 2-18-1022 | S | N—Ph | b1 | H | H |
| 2-18-1023 | S | N—Ph | b2 | H | H |
| 2-18-1024 | S | N—Ph | b3 | H | H |
| 2-18-1025 | S | N—Ph | H | H | a1 |
| 2-18-1026 | S | N—Ph | t-bu | H | a1 |
| 2-18-1027 | S | N—Ph | methyl | H | a1 |
| 2-18-1028 | S | N—Ph | b1 | H | a1 |
| 2-18-1029 | S | N—Ph | b2 | H | a1 |
| 2-18-1030 | S | N—Ph | b3 | H | a1 |
| 2-18-1031 | S | N—Ph | H | H | a2 |
| 2-18-1032 | S | N—Ph | t-bu | H | a2 |
| 2-18-1033 | S | N—Ph | methyl | H | a2 |
| 2-18-1034 | S | N—Ph | b1 | H | a2 |
| 2-18-1035 | S | N—Ph | b2 | H | a2 |
| 2-18-1036 | S | N—Ph | b3 | H | a2 |
| 2-18-1037 | S | N—Ph | H | H | a3 |
| 2-18-1038 | S | N—Ph | t-bu | H | a3 |
| 2-18-1039 | S | N—Ph | methyl | H | a3 |
| 2-18-1040 | S | N—Ph | b1 | H | a3 |
| 2-18-1041 | S | N—Ph | b2 | H | a3 |
| 2-18-1042 | S | N—Ph | b3 | H | a3 |
| 2-18-1043 | S | N—Ph | H | H | a4 |
| 2-18-1044 | S | N—Ph | t-bu | H | a4 |
| 2-18-1045 | S | N—Ph | methyl | H | a4 |
| 2-18-1046 | S | N—Ph | b1 | H | a4 |
| 2-18-1047 | S | N—Ph | b2 | H | a4 |
| 2-18-1048 | S | N—Ph | b3 | H | a4 |
| 2-18-1049 | S | N—Ph | H | H | a5 |
| 2-18-1050 | S | N—Ph | t-bu | H | a5 |
| 2-18-1051 | S | N—Ph | methyl | H | a5 |
| 2-18-1052 | S | N—Ph | b1 | H | a5 |
| 2-18-1053 | S | N—Ph | b2 | H | a5 |
| 2-18-1054 | S | N—Ph | b3 | H | a5 |
| 2-18-1055 | S | N—Ph | H | H | a6 |
| 2-18-1056 | S | N—Ph | t-bu | H | a6 |
| 2-18-1057 | S | N—Ph | methyl | H | a6 |
| 2-18-1058 | S | N—Ph | b1 | H | a6 |
| 2-18-1059 | S | N—Ph | b2 | H | a6 |
| 2-18-1060 | S | N—Ph | b3 | H | a6 |
| 2-18-1061 | S | N—Ph | H | H | b1 |
| 2-18-1062 | S | N—Ph | t-bu | H | b1 |
| 2-18-1063 | S | N—Ph | methyl | H | b1 |
| 2-18-1064 | S | N—Ph | b1 | H | b1 |
| 2-18-1065 | S | N—Ph | b2 | H | b1 |
| 2-18-1066 | S | N—Ph | b3 | H | b1 |
| 2-18-1067 | S | N—Ph | H | H | b2 |
| 2-18-1068 | S | N—Ph | t-bu | H | b2 |
| 2-18-1069 | S | N—Ph | methyl | H | b2 |
| 2-18-1070 | S | N—Ph | b1 | H | b2 |
| 2-18-1071 | S | N—Ph | b2 | H | b2 |
| 2-18-1072 | S | N—Ph | b3 | H | b2 |
| 2-18-1073 | S | N—Ph | H | H | b3 |
| 2-18-1074 | S | N—Ph | t-bu | H | b3 |
| 2-18-1075 | S | N—Ph | methyl | H | b3 |
| 2-18-1076 | S | N—Ph | b1 | H | b3 |
| 2-18-1077 | S | N—Ph | b2 | H | b3 |
| 2-18-1078 | S | N—Ph | b3 | H | b3 |
| 2-18-1079 | S | N—Ph | H | H | b4 |
| 2-18-1080 | S | N—Ph | t-bu | H | b4 |
| 2-18-1081 | S | N—Ph | methyl | H | b4 |
| 2-18-1082 | S | N—Ph | b1 | H | b4 |
| 2-18-1083 | S | N—Ph | b2 | H | b4 |
| 2-18-1084 | S | N—Ph | b3 | H | b4 |
| 2-18-1085 | S | N—Ph | H | H | b5 |
| 2-18-1086 | S | N—Ph | t-bu | H | b5 |
| 2-18-1087 | S | N—Ph | methyl | H | b5 |
| 2-18-1088 | S | N—Ph | b1 | H | b5 |
| 2-18-1089 | S | N—Ph | b2 | H | b5 |
| 2-18-1090 | S | N—Ph | b3 | H | b5 |
| 2-18-1091 | S | N—Ph | H | H | b6 |
| 2-18-1092 | S | N—Ph | t-bu | H | b6 |
| 2-18-1093 | S | N—Ph | methyl | H | b6 |
| 2-18-1094 | S | N—Ph | b1 | H | b6 |
| 2-18-1095 | S | N—Ph | b2 | H | b6 |
| 2-18-1096 | S | N—Ph | b3 | H | b6 |
| 2-18-1097 | S | N—Ph | H | H | b7 |
| 2-18-1098 | S | N—Ph | t-bu | H | b7 |
| 2-18-1099 | S | N—Ph | methyl | H | b7 |
| 2-18-1100 | S | N—Ph | b1 | H | b7 |
| 2-18-1101 | S | N—Ph | b2 | H | b7 |
| 2-18-1102 | S | N—Ph | b3 | H | b7 |
| 2-18-1103 | S | N—Ph | H | H | b8 |
| 2-18-1104 | S | N—Ph | t-bu | H | b8 |
| 2-18-1105 | S | N—Ph | methyl | H | b8 |
| 2-18-1106 | S | N—Ph | b1 | H | b8 |
| 2-18-1107 | S | N—Ph | b2 | H | b8 |
| 2-18-1108 | S | N—Ph | b3 | H | b8 |
| 2-18-1109 | S | N—Ph | H | H | b9 |
| 2-18-1110 | S | N—Ph | t-bu | H | b9 |
| 2-18-1111 | S | N—Ph | methyl | H | b9 |
| 2-18-1112 | S | N—Ph | b1 | H | b9 |
| 2-18-1113 | S | N—Ph | b2 | H | b9 |
| 2-18-1114 | S | N—Ph | b3 | H | b9 |
| 2-18-1115 | S | N—Ph | H | H | b10 |
| 2-18-1116 | S | N—Ph | t-bu | H | b10 |
| 2-18-1117 | S | N—Ph | methyl | H | b10 |
| 2-18-1118 | S | N—Ph | b1 | H | b10 |
| 2-18-1119 | S | N—Ph | b2 | H | b10 |
| 2-18-1120 | S | N—Ph | b3 | H | b10 |
| 2-18-1121 | S | N—Ph | H | a1 | H |
| 2-18-1122 | S | N—Ph | t-bu | a1 | H |
| 2-18-1123 | S | N—Ph | methyl | a1 | H |
| 2-18-1124 | S | N—Ph | b1 | a1 | H |
| 2-18-1125 | S | N—Ph | b2 | a1 | H |
| 2-18-1126 | S | N—Ph | b3 | a1 | H |
| 2-18-1127 | S | N—Ph | H | a2 | H |
| 2-18-1128 | S | N—Ph | t-bu | a2 | H |
| 2-18-1129 | S | N—Ph | methyl | a2 | H |
| 2-18-1130 | S | N—Ph | b1 | a2 | H |
| 2-18-1131 | S | N—Ph | b2 | a2 | H |
| 2-18-1132 | S | N—Ph | b3 | a2 | H |
| 2-18-1133 | S | N—Ph | H | a3 | H |
| 2-18-1134 | S | N—Ph | t-bu | a3 | H |
| 2-18-1135 | S | N—Ph | methyl | a3 | H |
| 2-18-1136 | S | N—Ph | b1 | a3 | H |
| 2-18-1137 | S | N—Ph | b2 | a3 | H |
| 2-18-1138 | S | N—Ph | b3 | a3 | H |
| 2-18-1139 | S | N—Ph | H | a4 | H |
| 2-18-1140 | S | N—Ph | t-bu | a4 | H |
| 2-18-1141 | S | N—Ph | methyl | a4 | H |
| 2-18-1142 | S | N—Ph | b1 | a4 | H |
| 2-18-1143 | S | N—Ph | b2 | a4 | H |
| 2-18-1144 | S | N—Ph | b3 | a4 | H |
| 2-18-1145 | S | N—Ph | H | a5 | H |
| 2-18-1146 | S | N—Ph | t-bu | a5 | H |
| 2-18-1147 | S | N—Ph | methyl | a5 | H |
| 2-18-1148 | S | N—Ph | b1 | a5 | H |
| 2-18-1149 | S | N—Ph | b2 | a5 | H |
| 2-18-1150 | S | N—Ph | b3 | a5 | H |
| 2-18-1151 | S | N—Ph | H | a6 | H |
| 2-18-1152 | S | N—Ph | t-bu | a6 | H |
| 2-18-1153 | S | N—Ph | methyl | a6 | H |
| 2-18-1154 | S | N—Ph | b1 | a6 | H |
| 2-18-1155 | S | N—Ph | b2 | a6 | H |
| 2-18-1156 | S | N—Ph | b3 | a6 | H |
| 2-18-1157 | S | N—Ph | H | b1 | H |
| 2-18-1158 | S | N—Ph | t-bu | b1 | H |
| 2-18-1159 | S | N—Ph | methyl | b1 | H |
| 2-18-1160 | S | N—Ph | b1 | b1 | H |
| 2-18-1161 | S | N—Ph | b2 | b1 | H |
| 2-18-1162 | S | N—Ph | b3 | b1 | H |
| 2-18-1163 | S | N—Ph | H | b2 | H |
| 2-18-1164 | S | N—Ph | t-bu | b2 | H |
| 2-18-1165 | S | N—Ph | methyl | b2 | H |
| 2-18-1166 | S | N—Ph | b1 | b2 | H |
| 2-18-1167 | S | N—Ph | b2 | b2 | H |
| 2-18-1168 | S | N—Ph | b3 | b2 | H |
| 2-18-1169 | S | N—Ph | H | b3 | H |
| 2-18-1170 | S | N—Ph | t-bu | b3 | H |
| 2-18-1171 | S | N—Ph | methyl | b3 | H |
| 2-18-1172 | S | N—Ph | b1 | b3 | H |
| 2-18-1173 | S | N—Ph | b2 | b3 | H |
| 2-18-1174 | S | N—Ph | b3 | b3 | H |
| 2-18-1175 | S | N—Ph | H | b4 | H |
| 2-18-1176 | S | N—Ph | t-bu | b4 | H |
| 2-18-1177 | S | N—Ph | methyl | b4 | H |
| 2-18-1178 | S | N—Ph | b1 | b4 | H |
| 2-18-1179 | S | N—Ph | b2 | b4 | H |
| 2-18-1180 | S | N—Ph | b3 | b4 | H |
| 2-18-1181 | S | N—Ph | H | b5 | H |
| 2-18-1182 | S | N—Ph | t-bu | b5 | H |
| 2-18-1183 | S | N—Ph | methyl | b5 | H |
| 2-18-1184 | S | N—Ph | b1 | b5 | H |
| 2-18-1185 | S | N—Ph | b2 | b5 | H |
| 2-18-1186 | S | N—Ph | b3 | b5 | H |
| 2-18-1187 | S | N—Ph | H | b6 | H |
| 2-18-1188 | S | N—Ph | t-bu | b6 | H |
| 2-18-1189 | S | N—Ph | methyl | b6 | H |
| 2-18-1190 | S | N—Ph | b1 | b6 | H |
| 2-18-1191 | S | N—Ph | b2 | b6 | H |
| 2-18-1192 | S | N—Ph | b3 | b6 | H |
| 2-18-1193 | S | N—Ph | H | b7 | H |
| 2-18-1194 | S | N—Ph | t-bu | b7 | H |
| 2-18-1195 | S | N—Ph | methyl | b7 | H |
| 2-18-1196 | S | N—Ph | b1 | b7 | H |
| 2-18-1197 | S | N—Ph | b2 | b7 | H |
| 2-18-1198 | S | N—Ph | b3 | b7 | H |
| 2-18-1199 | S | N—Ph | H | b8 | H |
| 2-18-1200 | S | N—Ph | t-bu | b8 | H |
| 2-18-1201 | S | N—Ph | methyl | b8 | H |
| 2-18-1202 | S | N—Ph | b1 | b8 | H |
| 2-18-1203 | S | N—Ph | b2 | b8 | H |
| 2-18-1204 | S | N—Ph | b3 | b8 | H |
| 2-18-1205 | S | N—Ph | H | b9 | H |
| 2-18-1206 | S | N—Ph | t-bu | b9 | H |
| 2-18-1207 | S | N—Ph | methyl | b9 | H |
| 2-18-1208 | S | N—Ph | b1 | b9 | H |
| 2-18-1209 | S | N—Ph | b2 | b9 | H |
| 2-18-1210 | S | N—Ph | b3 | b9 | H |
| 2-18-1211 | S | N—Ph | H | b10 | H |
| 2-18-1212 | S | N—Ph | t-bu | b10 | H |
| 2-18-1213 | S | N—Ph | methyl | b10 | H |
| 2-18-1214 | S | N—Ph | b1 | b10 | H |
| 2-18-1215 | S | N—Ph | b2 | b10 | H |
| 2-18-1216 | S | N—Ph | b3 | b10 | H |
| 2-18-1217 | S | N—Ph | H | a1 | a1 |
| 2-18-1218 | S | N—Ph | t-bu | a1 | a1 |
| 2-18-1219 | S | N—Ph | methyl | a1 | a1 |
| 2-18-1220 | S | N—Ph | b1 | a1 | a1 |
| 2-18-1221 | S | N—Ph | b2 | a1 | a1 |
| 2-18-1222 | S | N—Ph | b3 | a1 | a1 |
| 2-18-1223 | S | N—Ph | H | a2 | a2 |
| 2-18-1224 | S | N—Ph | t-bu | a2 | a2 |
| 2-18-1225 | S | N—Ph | methyl | a2 | a2 |
| 2-18-1226 | S | N—Ph | b1 | a2 | a2 |
| 2-18-1227 | S | N—Ph | b2 | a2 | a2 |
| 2-18-1228 | S | N—Ph | b3 | a2 | a2 |
| 2-18-1229 | S | N—Ph | H | a3 | a3 |
| 2-18-1230 | S | N—Ph | t-bu | a3 | a3 |
| 2-18-1231 | S | N—Ph | methyl | a3 | a3 |
| 2-18-1232 | S | N—Ph | b1 | a3 | a3 |
| 2-18-1233 | S | N—Ph | b2 | a3 | a3 |
| 2-18-1234 | S | N—Ph | b3 | a3 | a3 |
| 2-18-1235 | S | N—Ph | H | b1 | b1 |
| 2-18-1236 | S | N—Ph | t-bu | b1 | b1 |
| 2-18-1237 | S | N—Ph | methyl | b1 | b1 |
| 2-18-1238 | S | N—Ph | b1 | b1 | b1 |
| 2-18-1239 | S | N—Ph | b2 | b1 | b1 |
| 2-18-1240 | S | N—Ph | b3 | b1 | b1 |
| 2-18-1241 | S | N—Ph | H | b2 | b2 |
| 2-18-1242 | S | N—Ph | t-bu | b2 | b2 |
| 2-18-1243 | S | N—Ph | methyl | b2 | b2 |
| 2-18-1244 | S | N—Ph | b1 | b2 | b2 |
| 2-18-1245 | S | N—Ph | b2 | b2 | b2 |
| 2-18-1246 | S | N—Ph | b3 | b2 | b2 |
| 2-18-1247 | S | N—Ph | H | b3 | b3 |
| 2-18-1248 | S | N—Ph | t-bu | b3 | b3 |
| 2-18-1249 | S | N—Ph | methyl | b3 | b3 |
| 2-18-1250 | S | N—Ph | b1 | b3 | b3 |
| 2-18-1251 | S | N—Ph | b2 | b3 | b3 |
| 2-18-1252 | S | N—Ph | b3 | b3 | b3 |
| 2-18-1253 | S | N—Ph | H | b5 | b5 |
| 2-18-1254 | S | N—Ph | t-bu | b5 | b5 |
| 2-18-1255 | S | N—Ph | methyl | b5 | b5 |
| 2-18-1256 | S | N—Ph | b1 | b5 | b5 |
| 2-18-1257 | S | N—Ph | b2 | b5 | b5 |
| 2-18-1258 | S | N—Ph | b3 | b5 | b5 |
| 2-18-1259 | S | N—Ph | H | b6 | b6 |
| 2-18-1260 | S | N—Ph | t-bu | b6 | b6 |
| 2-18-1261 | S | N—Ph | methyl | b6 | b6 |
| 2-18-1262 | S | N—Ph | b1 | b6 | b6 |
| 2-18-1263 | S | N—Ph | b2 | b6 | b6 |
| 2-18-1264 | S | N—Ph | b3 | b6 | b6 |
| 2-18-1265 | S | N—Ph | H | b7 | b7 |
| 2-18-1266 | S | N—Ph | t-bu | b7 | b7 |
| 2-18-1267 | S | N—Ph | methyl | b7 | b7 |
| 2-18-1268 | S | N—Ph | b1 | b7 | b7 |
| 2-18-1269 | S | N—Ph | b2 | b7 | b7 |
| 2-18-1270 | S | N—Ph | b3 | b7 | b7 |

### Group 2-19

The compounds are represented by the formula 2-19 below, wherein CY₁ and CY₃ are each a benzene group, X₁ is B, and Y1, Y2, Y3, R2b, (R1)a1, (R3)a3 are each shown in the table 19 below,

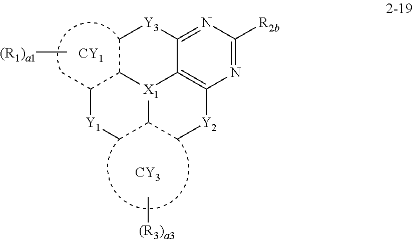

TABLE 19

| Compound No. | Y1 | Y2/Y3 | R2b | (R1)a1 | (R3)a3 |
|---|---|---|---|---|---|
| 2-19-001 | N—Ph | N Ph | H | H | H |
| 2-19-002 | N Ph | N Ph | t-bu | H | H |
| 2-19-003 | N Ph | N Ph | methyl | H | H |
| 2-19-004 | N Ph | N Ph | t-bu | t-bu | t-bu |
| 2-19-005 | N Ph | N Ph | methyl | methyl | methyl |
| 2-19-006 | N Ph | N Ph | b1 | H | H |
| 2-19-007 | N Ph | N Ph | b2 | H | H |
| 2-19-008 | N Ph | N Ph | b3 | H | H |
| 2-19-009 | N Ph | N Ph | H | H | a1 |
| 2-19-010 | N Ph | N Ph | t-bu | H | a1 |
| 2-19-011 | N Ph | N Ph | methyl | H | a1 |
| 2-19-012 | N Ph | N Ph | b1 | H | a1 |
| 2-19-013 | N Ph | N Ph | b2 | H | a1 |
| 2-19-014 | N Ph | N Ph | b3 | H | a1 |
| 2-19-015 | N Ph | N Ph | H | H | a2 |
| 2-19-016 | N Ph | N Ph | t-bu | H | a2 |
| 2-19-017 | N Ph | N Ph | methyl | H | a2 |
| 2-19-018 | N Ph | N Ph | b1 | H | a2 |
| 2-19-019 | N Ph | N Ph | b2 | H | a2 |
| 2-19-020 | N Ph | N Ph | b3 | H | a2 |
| 2-19-021 | N Ph | N Ph | H | H | a3 |
| 2-19-022 | N Ph | N Ph | t-bu | H | a3 |
| 2-19-023 | N Ph | N Ph | methyl | H | a3 |
| 2-19-024 | N Ph | N Ph | b1 | H | a3 |
| 2-19-025 | N Ph | N Ph | b2 | H | a3 |
| 2-19-026 | N Ph | N Ph | b3 | H | a3 |
| 2-19-027 | N Ph | N Ph | H | H | a4 |
| 2-19-028 | N Ph | N Ph | t-bu | H | a4 |
| 2-19-029 | N Ph | N Ph | methyl | H | a4 |
| 2-19-030 | N Ph | N Ph | b1 | H | a4 |
| 2-19-031 | N Ph | N Ph | b2 | H | a4 |
| 2-19-032 | N Ph | N Ph | b3 | H | a4 |
| 2-19-033 | N Ph | N Ph | H | H | a5 |
| 2-19-034 | N Ph | N Ph | t-bu | H | a5 |
| 2-19-035 | N Ph | N Ph | methyl | H | a5 |
| 2-19-036 | N Ph | N Ph | b1 | H | a5 |
| 2-19-037 | N Ph | N Ph | b2 | H | a5 |
| 2-19-038 | N Ph | N Ph | b3 | H | a5 |
| 2-19-039 | N Ph | N Ph | H | H | a6 |
| 2-19-040 | N Ph | N Ph | t-bu | H | a6 |
| 2-19-041 | N Ph | N Ph | methyl | H | a6 |
| 2-19-042 | N Ph | N Ph | b1 | H | a6 |
| 2-19-043 | N Ph | N Ph | b2 | H | a6 |
| 2-19-044 | N Ph | N Ph | b3 | H | a6 |
| 2-19-045 | N Ph | N Ph | H | H | b1 |
| 2-19-046 | N Ph | N Ph | t-bu | H | b1 |
| 2-19-047 | N Ph | N Ph | methyl | H | b1 |
| 2-19-048 | N Ph | N Ph | b1 | H | b1 |
| 2-19-049 | N Ph | N Ph | b2 | H | b1 |
| 2-19-050 | N Ph | N Ph | b3 | H | b1 |
| 2-19-051 | N Ph | N Ph | H | H | b2 |
| 2-19-052 | N Ph | N Ph | t-bu | H | b2 |
| 2-19-053 | N Ph | N Ph | methyl | H | b2 |
| 2-19-054 | N Ph | N Ph | b1 | H | b2 |
| 2-19-055 | N Ph | N Ph | b2 | H | b2 |
| 2-19-056 | N Ph | N Ph | b3 | H | b2 |
| 2-19-057 | N Ph | N Ph | H | H | b3 |
| 2-19-058 | N Ph | N Ph | t-bu | H | b3 |
| 2-19-059 | N Ph | N Ph | methyl | H | b3 |
| 2-19-060 | N Ph | N Ph | b1 | H | b3 |
| 2-19-061 | N Ph | N Ph | b2 | H | b3 |
| 2-19-062 | N Ph | N Ph | b3 | H | b3 |
| 2-19-063 | N Ph | N Ph | H | H | b4 |
| 2-19-064 | N Ph | N Ph | t-bu | H | b4 |
| 2-19-065 | N Ph | N Ph | methyl | H | b4 |
| 2-19-066 | N Ph | N Ph | b1 | H | b4 |
| 2-19-067 | N Ph | N Ph | b2 | H | b4 |
| 2-19-068 | N Ph | N Ph | b3 | H | b4 |
| 2-19-069 | N Ph | N Ph | H | H | b5 |
| 2-19-070 | N Ph | N Ph | t-bu | H | b5 |
| 2-19-071 | N Ph | N Ph | methyl | H | b5 |
| 2-19-072 | N Ph | N Ph | b1 | H | b5 |
| 2-19-073 | N Ph | N Ph | b2 | H | b5 |
| 2-19-074 | N Ph | N Ph | b3 | H | b5 |
| 2-19-075 | N Ph | N Ph | H | H | b6 |
| 2-19-076 | N Ph | N Ph | t-bu | H | b6 |
| 2-19-077 | N Ph | N Ph | methyl | H | b6 |
| 2-19-078 | N Ph | N Ph | b1 | H | b6 |
| 2-19-079 | N Ph | N Ph | b2 | H | b6 |
| 2-19-080 | N Ph | N Ph | b3 | H | b6 |
| 2-19-081 | N Ph | N Ph | H | H | b7 |
| 2-19-082 | N Ph | N Ph | t-bu | H | b7 |
| 2-19-083 | N Ph | N Ph | methyl | H | b7 |
| 2-19-084 | N Ph | N Ph | b1 | H | b7 |
| 2-19-085 | N Ph | N Ph | b2 | H | b7 |
| 2-19-086 | N Ph | N Ph | b3 | H | b7 |
| 2-19-087 | N Ph | N Ph | H | H | b8 |
| 2-19-088 | N Ph | N Ph | t-bu | H | b8 |
| 2-19-089 | N Ph | N Ph | methyl | H | b8 |
| 2-19-090 | N Ph | N Ph | b1 | H | b8 |
| 2-19-091 | N Ph | N Ph | b2 | H | b8 |
| 2-19-092 | N Ph | N Ph | b3 | H | b8 |
| 2-19-093 | N Ph | N Ph | H | H | b9 |
| 2-19-094 | N Ph | N Ph | t-bu | H | b9 |
| 2-19-095 | N Ph | N Ph | methyl | H | b9 |
| 2-19-096 | N Ph | N Ph | b1 | H | b9 |
| 2-19-097 | N Ph | N Ph | b2 | H | b9 |
| 2-19-098 | N Ph | N Ph | b3 | H | b9 |
| 2-19-099 | N Ph | N Ph | H | H | b10 |
| 2-19-100 | N Ph | N Ph | t-bu | H | b10 |
| 2-19-101 | N Ph | N Ph | methyl | H | b10 |
| 2-19-102 | N Ph | N Ph | b1 | H | b10 |
| 2-19-103 | N Ph | N Ph | b2 | H | b10 |
| 2-19-104 | N Ph | N Ph | b3 | H | b10 |
| 2-19-105 | N Ph | N Ph | H | a1 | H |
| 2-19-106 | N Ph | N Ph | t-bu | a1 | H |
| 2-19-107 | N Ph | N Ph | methyl | a1 | H |
| 2-19-108 | N Ph | N Ph | b1 | a1 | H |
| 2-19-109 | N Ph | N Ph | b2 | a1 | H |
| 2-19-110 | N Ph | N Ph | b3 | a1 | H |
| 2-19-111 | N Ph | N Ph | H | a2 | H |
| 2-19-112 | N Ph | N Ph | t-bu | a2 | H |
| 2-19-113 | N Ph | N Ph | methyl | a2 | H |
| 2-19-114 | N Ph | N Ph | b1 | a2 | H |
| 2-19-115 | N Ph | N Ph | b2 | a2 | H |
| 2-19-116 | N Ph | N Ph | b3 | a2 | H |
| 2-19-117 | N Ph | N Ph | H | a3 | H |
| 2-19-118 | N Ph | N Ph | t-bu | a3 | H |
| 2-19-119 | N Ph | N Ph | methyl | a3 | H |
| 2-19-120 | N Ph | N Ph | b1 | a3 | H |
| 2-19-121 | N Ph | N Ph | b2 | a3 | H |
| 2-19-122 | N Ph | N Ph | b3 | a3 | H |
| 2-19-123 | N Ph | N Ph | H | a4 | H |
| 2-19-124 | N Ph | N Ph | t-bu | a4 | H |
| 2-19-125 | N Ph | N Ph | methyl | a4 | H |
| 2-19-126 | N Ph | N Ph | b1 | a4 | H |
| 2-19-127 | N Ph | N Ph | b2 | a4 | H |
| 2-19-128 | N Ph | N Ph | b3 | a4 | H |
| 2-19-129 | N Ph | N Ph | H | a5 | H |
| 2-19-130 | N Ph | N Ph | t-bu | a5 | H |
| 2-19-131 | N Ph | N Ph | methyl | a5 | H |
| 2-19-132 | N Ph | N Ph | b1 | a5 | H |
| 2-19-133 | N Ph | N Ph | b2 | a5 | H |
| 2-19-134 | N Ph | N Ph | b3 | a5 | H |
| 2-19-135 | N Ph | N Ph | H | a6 | H |
| 2-19-136 | N Ph | N Ph | t-bu | a6 | H |
| 2-19-137 | N Ph | N Ph | methyl | a6 | H |
| 2-19-138 | N Ph | N Ph | b1 | a6 | H |
| 2-19-139 | N Ph | N Ph | b2 | a6 | H |
| 2-19-140 | N Ph | N Ph | b3 | a6 | H |
| 2-19-141 | N Ph | N Ph | H | b1 | H |
| 2-19-142 | N Ph | N Ph | t-bu | b1 | H |
| 2-19-143 | N Ph | N Ph | methyl | b1 | H |
| 2-19-144 | N Ph | N Ph | b1 | b1 | H |
| 2-19-145 | N Ph | N Ph | b2 | b1 | H |
| 2-19-146 | N Ph | N Ph | b3 | b1 | H |
| 2-19-147 | N Ph | N Ph | H | b2 | H |
| 2-19-148 | N Ph | N Ph | t-bu | b2 | H |
| 2-19-149 | N Ph | N Ph | methyl | b2 | H |
| 2-19-150 | N Ph | N Ph | b1 | b2 | H |
| 2-19-151 | N Ph | N Ph | b2 | b2 | H |
| 2-19-152 | N Ph | N Ph | b3 | b2 | H |
| 2-19-153 | N Ph | N Ph | H | b3 | H |
| 2-19-154 | N Ph | N Ph | t-bu | b3 | H |
| 2-19-155 | N Ph | N Ph | methyl | b3 | H |
| 2-19-156 | N Ph | N Ph | b1 | b3 | H |
| 2-19-157 | N Ph | N Ph | b2 | b3 | H |
| 2-19-158 | N Ph | N Ph | b3 | b3 | H |
| 2-19-159 | N Ph | N Ph | H | b4 | H |
| 2-19-160 | N Ph | N Ph | t-bu | b4 | H |
| 2-19-161 | N Ph | N Ph | methyl | b4 | H |
| 2-19-162 | N Ph | N Ph | b1 | b4 | H |
| 2-19-163 | N Ph | N Ph | b2 | b4 | H |
| 2-19-164 | N Ph | N Ph | b3 | b4 | H |
| 2-19-165 | N Ph | N Ph | H | b5 | H |
| 2-19-166 | N Ph | N Ph | t-bu | b5 | H |
| 2-19-167 | N Ph | N Ph | methyl | b5 | H |
| 2-19-168 | N Ph | N Ph | b1 | b5 | H |
| 2-19-169 | N Ph | N Ph | b2 | b5 | H |
| 2-19-170 | N Ph | N Ph | b3 | b5 | H |
| 2-19-171 | N Ph | N Ph | H | b6 | H |
| 2-19-172 | N Ph | N Ph | t-bu | b6 | H |
| 2-19-173 | N Ph | N Ph | methyl | b6 | H |
| 2-19-174 | N Ph | N Ph | b1 | b6 | H |
| 2-19-175 | N Ph | N Ph | b2 | b6 | H |
| 2-19-176 | N Ph | N Ph | b3 | b6 | H |
| 2-19-177 | N Ph | N Ph | H | b7 | H |
| 2-19-178 | N Ph | N Ph | t-bu | b7 | H |
| 2-19-179 | N Ph | N Ph | methyl | b7 | H |
| 2-19-180 | N Ph | N Ph | b1 | b7 | H |
| 2-19-181 | N Ph | N Ph | b2 | b7 | H |
| 2-19-182 | N Ph | N Ph | b3 | b7 | H |
| 2-19-183 | N Ph | N Ph | H | b8 | H |
| 2-19-184 | N Ph | N Ph | t-bu | b8 | H |
| 2-19-185 | N Ph | N Ph | methyl | b8 | H |
| 2-19-186 | N Ph | N Ph | b1 | b8 | H |
| 2-19-187 | N Ph | N Ph | b2 | b8 | H |
| 2-19-188 | N Ph | N Ph | b3 | b8 | H |
| 2-19-189 | N Ph | N Ph | H | b9 | H |
| 2-19-190 | N Ph | N Ph | t-bu | b9 | H |
| 2-19-191 | N Ph | N Ph | methyl | b9 | H |
| 2-19-192 | N Ph | N Ph | b1 | b9 | H |
| 2-19-193 | N Ph | N Ph | b2 | b9 | H |
| 2-19-194 | N Ph | N Ph | b3 | b9 | H |
| 2-19-195 | N Ph | N Ph | H | b10 | H |
| 2-19-196 | N Ph | N Ph | t-bu | b10 | H |
| 2-19-197 | N Ph | N Ph | methyl | b10 | H |
| 2-19-198 | N Ph | N Ph | b1 | b10 | H |
| 2-19-199 | N Ph | N Ph | b2 | b10 | H |
| 2-19-200 | N Ph | N Ph | b3 | b10 | H |
| 2-19-201 | N Ph | N Ph | H | a1 | a1 |
| 2-19-202 | N Ph | N Ph | t-bu | a1 | a1 |
| 2-19-203 | N Ph | N Ph | methyl | a1 | a1 |
| 2-19-204 | N Ph | N Ph | b1 | a1 | a1 |
| 2-19-205 | N Ph | N Ph | b2 | a1 | a1 |
| 2-19-206 | N Ph | N Ph | b3 | a1 | a1 |
| 2-19-207 | N Ph | N Ph | H | a2 | a2 |
| 2-19-208 | N Ph | N Ph | t-bu | a2 | a2 |
| 2-19-209 | N Ph | N Ph | methyl | a2 | a2 |
| 2-19-210 | N Ph | N Ph | b1 | a2 | a2 |
| 2-19-211 | N Ph | N Ph | b2 | a2 | a2 |
| 2-19-212 | N Ph | N Ph | b3 | a2 | a2 |
| 2-19-213 | N Ph | N Ph | H | a3 | a3 |
| 2-19-214 | N Ph | N Ph | t-bu | a3 | a3 |
| 2-19-215 | N Ph | N Ph | methyl | a3 | a3 |
| 2-19-216 | N Ph | N Ph | b1 | a3 | a3 |
| 2-19-217 | N Ph | N Ph | b2 | a3 | a3 |
| 2-19-218 | N Ph | N Ph | b3 | a3 | a3 |
| 2-19-219 | N Ph | N Ph | H | b1 | b1 |
| 2-19-220 | N Ph | N Ph | t-bu | b1 | b1 |
| 2-19-221 | N Ph | N Ph | methyl | b1 | b1 |
| 2-19-222 | N Ph | N Ph | b1 | b1 | b1 |
| 2-19-223 | N Ph | N Ph | b2 | b1 | b1 |
| 2-19-224 | N Ph | N Ph | b3 | b1 | b1 |
| 2-19-225 | N Ph | N Ph | H | b2 | b2 |
| 2-19-226 | N Ph | N Ph | t-bu | b2 | b2 |
| 2-19-227 | N Ph | N Ph | methyl | b2 | b2 |
| 2-19-228 | N Ph | N Ph | b1 | b2 | b2 |
| 2-19-229 | N Ph | N Ph | b2 | b2 | b2 |
| 2-19-230 | N Ph | N Ph | b3 | b2 | b2 |
| 2-19-231 | N Ph | N Ph | H | b3 | b3 |
| 2-19-232 | N Ph | N Ph | t-bu | b3 | b3 |
| 2-19-233 | N Ph | N Ph | methyl | b3 | b3 |
| 2-19-234 | N Ph | N Ph | b1 | b3 | b3 |
| 2-19-235 | N Ph | N Ph | b2 | b3 | b3 |
| 2-19-236 | N Ph | N Ph | b3 | b3 | b3 |
| 2-19-237 | N Ph | N Ph | H | b5 | b5 |
| 2-19-238 | N Ph | N Ph | t-bu | b5 | b5 |
| 2-19-239 | N Ph | N Ph | methyl | b5 | b5 |
| 2-19-240 | N Ph | N Ph | b1 | b5 | b5 |
| 2-19-241 | N Ph | N Ph | b2 | b5 | b5 |
| 2-19-242 | N Ph | N Ph | b3 | b5 | b5 |
| 2-19-243 | N Ph | N Ph | H | b6 | b6 |
| 2-19-244 | N Ph | N Ph | t-bu | b6 | b6 |
| 2-19-245 | N Ph | N Ph | methyl | b6 | b6 |
| 2-19-246 | N Ph | N Ph | b1 | b6 | b6 |
| 2-19-247 | N Ph | N Ph | b2 | b6 | b6 |
| 2-19-248 | N Ph | N Ph | b3 | b6 | b6 |
| 2-19-249 | N Ph | N Ph | H | b7 | b7 |
| 2-19-250 | N Ph | N Ph | t-bu | b7 | b7 |
| 2-19-251 | N Ph | N Ph | methyl | b7 | b7 |
| 2-19-252 | N Ph | N Ph | b1 | b7 | b7 |
| 2-19-253 | N Ph | N Ph | b2 | b7 | b7 |
| 2-19-254 | N Ph | N Ph | b3 | b7 | b7 |
| 2-19-255 | N-tolyl | N-tolyl | H | H | H |
| 2-19-256 | N-tolyl | N-tolyl | t-bu | H | H |
| 2-19-257 | N-tolyl | N-tolyl | methyl | H | H |
| 2-19-258 | N-tolyl | N-tolyl | t-bu | t-bu | t-bu |
| 2-19-259 | N-tolyl | N-tolyl | methyl | methyl | methyl |
| 2-19-260 | N-tolyl | N-tolyl | b1 | H | H |
| 2-19-261 | N-tolyl | N-tolyl | b2 | H | H |
| 2-19-262 | N-tolyl | N-tolyl | b3 | H | H |
| 2-19-263 | N-tolyl | N-tolyl | H | H | a1 |
| 2-19-264 | N-tolyl | N-tolyl | t-bu | H | a1 |
| 2-19-265 | N-tolyl | N-tolyl | methyl | H | a1 |
| 2-19-266 | N-tolyl | N-tolyl | b1 | H | a1 |
| 2-19-267 | N-tolyl | N-tolyl | b2 | H | a1 |
| 2-19-268 | N-tolyl | N-tolyl | b3 | H | a1 |
| 2-19-269 | N-tolyl | N-tolyl | H | H | a2 |
| 2-19-270 | N-tolyl | N-tolyl | t-bu | H | a2 |
| 2-19-271 | N-tolyl | N-tolyl | methyl | H | a2 |
| 2-19-272 | N-tolyl | N-tolyl | b1 | H | a2 |
| 2-19-273 | N-tolyl | N-tolyl | b2 | H | a2 |
| 2-19-274 | N-tolyl | N-tolyl | b3 | H | a2 |
| 2-19-275 | N-tolyl | N-tolyl | H | H | a3 |
| 2-19-276 | N-tolyl | N-tolyl | t-bu | H | a3 |
| 2-19-277 | N-tolyl | N-tolyl | methyl | H | a3 |
| 2-19-278 | N-tolyl | N-tolyl | b1 | H | a3 |
| 2-19-279 | N-tolyl | N-tolyl | b2 | H | a3 |
| 2-19-280 | N-tolyl | N-tolyl | b3 | H | a3 |
| 2-19-281 | N-tolyl | N-tolyl | H | H | a4 |
| 2-19-282 | N-tolyl | N-tolyl | t-bu | H | a4 |
| 2-19-283 | N-tolyl | N-tolyl | methyl | H | a4 |
| 2-19-284 | N-tolyl | N-tolyl | b1 | H | a4 |
| 2-19-285 | N-tolyl | N-tolyl | b2 | H | a4 |
| 2-19-286 | N-tolyl | N-tolyl | b3 | H | a4 |
| 2-19-287 | N-tolyl | N-tolyl | H | H | a5 |
| 2-19-288 | N-tolyl | N-tolyl | t-bu | H | a5 |
| 2-19-289 | N-tolyl | N-tolyl | methyl | H | a5 |
| 2-19-290 | N-tolyl | N-tolyl | b1 | H | a5 |
| 2-19-291 | N-tolyl | N-tolyl | b2 | H | a5 |
| 2-19-292 | N-tolyl | N-tolyl | b3 | H | a5 |
| 2-19-293 | N-tolyl | N-tolyl | H | H | a6 |
| 2-19-294 | N-tolyl | N-tolyl | t-bu | H | a6 |
| 2-19-295 | N-tolyl | N-tolyl | methyl | H | a6 |
| 2-19-296 | N-tolyl | N-tolyl | b1 | H | a6 |
| 2-19-297 | N-tolyl | N-tolyl | b2 | H | a6 |
| 2-19-298 | N-tolyl | N-tolyl | b3 | H | a6 |
| 2-19-299 | N-tolyl | N-tolyl | H | H | b1 |
| 2-19-300 | N-tolyl | N-tolyl | t-bu | H | b1 |
| 2-19-301 | N-tolyl | N-tolyl | methyl | H | b1 |
| 2-19-302 | N-tolyl | N-tolyl | b1 | H | b1 |
| 2-19-303 | N-tolyl | N-tolyl | b2 | H | b1 |
| 2-19-304 | N-tolyl | N-tolyl | b3 | H | b1 |
| 2-19-305 | N-tolyl | N-tolyl | H | H | b2 |
| 2-19-306 | N-tolyl | N-tolyl | t-bu | H | b2 |
| 2-19-307 | N-tolyl | N-tolyl | methyl | H | b2 |
| 2-19-308 | N-tolyl | N-tolyl | b1 | H | b2 |
| 2-19-309 | N-tolyl | N-tolyl | b2 | H | b2 |
| 2-19-310 | N-tolyl | N-tolyl | b3 | H | b2 |
| 2-19-311 | N-tolyl | N-tolyl | H | H | b3 |
| 2-19-312 | N-tolyl | N-tolyl | t-bu | H | b3 |
| 2-19-313 | N-tolyl | N-tolyl | methyl | H | b3 |
| 2-19-314 | N-tolyl | N-tolyl | b1 | H | b3 |
| 2-19-315 | N-tolyl | N-tolyl | b2 | H | b3 |
| 2-19-316 | N-tolyl | N-tolyl | b3 | H | b3 |
| 2-19-317 | N-tolyl | N-tolyl | H | H | b4 |
| 2-19-318 | N-tolyl | N-tolyl | t-bu | H | b4 |
| 2-19-319 | N-tolyl | N-tolyl | methyl | H | b4 |
| 2-19-320 | N-tolyl | N-tolyl | b1 | H | b4 |
| 2-19-321 | N-tolyl | N-tolyl | b2 | H | b4 |
| 2-19-322 | N-tolyl | N-tolyl | b3 | H | b4 |
| 2-19-323 | N-tolyl | N-tolyl | H | H | b5 |
| 2-19-324 | N-tolyl | N-tolyl | t-bu | H | b5 |
| 2-19-325 | N-tolyl | N-tolyl | methyl | H | b5 |
| 2-19-326 | N-tolyl | N-tolyl | b1 | H | b5 |
| 2-19-327 | N-tolyl | N-tolyl | b2 | H | b5 |
| 2-19-328 | N-tolyl | N-tolyl | b3 | H | b5 |
| 2-19-329 | N-tolyl | N-tolyl | H | H | b6 |
| 2-19-330 | N-tolyl | N-tolyl | t-bu | H | b6 |
| 2-19-331 | N-tolyl | N-tolyl | methyl | H | b6 |
| 2-19-332 | N-tolyl | N-tolyl | b1 | H | b6 |
| 2-19-333 | N-tolyl | N-tolyl | b2 | H | b6 |
| 2-19-334 | N-tolyl | N-tolyl | b3 | H | b6 |
| 2-19-335 | N-tolyl | N-tolyl | H | H | b7 |
| 2-19-336 | N-tolyl | N-tolyl | t-bu | H | b7 |
| 2-19-337 | N-tolyl | N-tolyl | methyl | H | b7 |
| 2-19-338 | N-tolyl | N-tolyl | b1 | H | b7 |
| 2-19-339 | N-tolyl | N-tolyl | b2 | H | b7 |
| 2-19-340 | N-tolyl | N-tolyl | b3 | H | b7 |
| 2-19-341 | N-tolyl | N-tolyl | H | H | b8 |
| 2-19-342 | N-tolyl | N-tolyl | t-bu | H | b8 |
| 2-19-343 | N-tolyl | N-tolyl | methyl | H | b8 |
| 2-19-344 | N-tolyl | N-tolyl | b1 | H | b8 |
| 2-19-345 | N-tolyl | N-tolyl | b2 | H | b8 |
| 2-19-346 | N-tolyl | N-tolyl | b3 | H | b8 |
| 2-19-347 | N-tolyl | N-tolyl | H | H | b9 |
| 2-19-348 | N-tolyl | N-tolyl | t-bu | H | b9 |
| 2-19-349 | N-tolyl | N-tolyl | methyl | H | b9 |
| 2-19-350 | N-tolyl | N-tolyl | b1 | H | b9 |
| 2-19-351 | N-tolyl | N-tolyl | b2 | H | b9 |
| 2-19-352 | N-tolyl | N-tolyl | b3 | H | b9 |
| 2-19-353 | N-tolyl | N-tolyl | H | H | b10 |
| 2-19-354 | N-tolyl | N-tolyl | t-bu | H | b10 |
| 2-19-355 | N-tolyl | N-tolyl | methyl | H | b10 |
| 2-19-356 | N-tolyl | N-tolyl | b1 | H | b10 |
| 2-19-357 | N-tolyl | N-tolyl | b2 | H | b10 |
| 2-19-358 | N-tolyl | N-tolyl | b3 | H | b10 |
| 2-19-359 | N-tolyl | N-tolyl | H | a1 | H |
| 2-19-360 | N-tolyl | N-tolyl | t-bu | a1 | H |
| 2-19-361 | N-tolyl | N-tolyl | methyl | a1 | H |
| 2-19-362 | N-tolyl | N-tolyl | b1 | a1 | H |
| 2-19-363 | N-tolyl | N-tolyl | b2 | a1 | H |
| 2-19-364 | N-tolyl | N-tolyl | b3 | a1 | H |
| 2-19-365 | N-tolyl | N-tolyl | H | a2 | H |
| 2-19-366 | N-tolyl | N-tolyl | t-bu | a2 | H |
| 2-19-367 | N-tolyl | N-tolyl | methyl | a2 | H |
| 2-19-368 | N-tolyl | N-tolyl | b1 | a2 | H |
| 2-19-369 | N-tolyl | N-tolyl | b2 | a2 | H |
| 2-19-370 | N-tolyl | N-tolyl | b3 | a2 | H |
| 2-19-371 | N-tolyl | N-tolyl | H | a3 | H |
| 2-19-372 | N-tolyl | N-tolyl | t-bu | a3 | H |
| 2-19-373 | N-tolyl | N-tolyl | methyl | a3 | H |
| 2-19-374 | N-tolyl | N-tolyl | b1 | a3 | H |
| 2-19-375 | N-tolyl | N-tolyl | b2 | a3 | H |
| 2-19-376 | N-tolyl | N-tolyl | b3 | a3 | H |
| 2-19-377 | N-tolyl | N-tolyl | H | a4 | H |
| 2-19-378 | N-tolyl | N-tolyl | t-bu | a4 | H |
| 2-19-379 | N-tolyl | N-tolyl | methyl | a4 | H |
| 2-19-380 | N-tolyl | N-tolyl | b1 | a4 | H |
| 2-19-381 | N-tolyl | N-tolyl | b2 | a4 | H |
| 2-19-382 | N-tolyl | N-tolyl | b3 | a4 | H |
| 2-19-383 | N-tolyl | N-tolyl | H | a5 | H |
| 2-19-384 | N-tolyl | N-tolyl | t-bu | a5 | H |
| 2-19-385 | N-tolyl | N-tolyl | methyl | a5 | H |
| 2-19-386 | N-tolyl | N-tolyl | b1 | a5 | H |
| 2-19-387 | N-tolyl | N-tolyl | b2 | a5 | H |
| 2-19-388 | N-tolyl | N-tolyl | b3 | a5 | H |
| 2-19-389 | N-tolyl | N-tolyl | H | a6 | H |
| 2-19-390 | N-tolyl | N-tolyl | t-bu | a6 | H |
| 2-19-391 | N-tolyl | N-tolyl | methyl | a6 | H |
| 2-19-392 | N-tolyl | N-tolyl | b1 | a6 | H |
| 2-19-393 | N-tolyl | N-tolyl | b2 | a6 | H |
| 2-19-394 | N-tolyl | N-tolyl | b3 | a6 | H |
| 2-19-395 | N-tolyl | N-tolyl | H | b1 | H |
| 2-19-396 | N-tolyl | N-tolyl | t-bu | b1 | H |
| 2-19-397 | N-tolyl | N-tolyl | methyl | b1 | H |
| 2-19-398 | N-tolyl | N-tolyl | b1 | b1 | H |
| 2-19-399 | N-tolyl | N-tolyl | b2 | b1 | H |
| 2-19-400 | N-tolyl | N-tolyl | b3 | b1 | H |
| 2-19-401 | N-tolyl | N-tolyl | H | b2 | H |
| 2-19-402 | N-tolyl | N-tolyl | t-bu | b2 | H |
| 2-19-403 | N-tolyl | N-tolyl | methyl | b2 | H |
| 2-19-404 | N-tolyl | N-tolyl | b1 | b2 | H |
| 2-19-405 | N-tolyl | N-tolyl | b2 | b2 | H |
| 2-19-406 | N-tolyl | N-tolyl | b3 | b2 | H |
| 2-19-407 | N-tolyl | N-tolyl | H | b3 | H |
| 2-19-408 | N-tolyl | N-tolyl | t-bu | b3 | H |
| 2-19-409 | N-tolyl | N-tolyl | methyl | b3 | H |
| 2-19-410 | N-tolyl | N-tolyl | b1 | b3 | H |
| 2-19-411 | N-tolyl | N-tolyl | b2 | b3 | H |
| 2-19-412 | N-tolyl | N-tolyl | b3 | b3 | H |
| 2-19-413 | N-tolyl | N-tolyl | H | b4 | H |
| 2-19-414 | N-tolyl | N-tolyl | t-bu | b4 | H |
| 2-19-415 | N-tolyl | N-tolyl | methyl | b4 | H |
| 2-19-416 | N-tolyl | N-tolyl | b1 | b4 | H |
| 2-19-417 | N-tolyl | N-tolyl | b2 | b4 | H |
| 2-19-418 | N-tolyl | N-tolyl | b3 | b4 | H |
| 2-19-419 | N-tolyl | N-tolyl | H | b5 | H |
| 2-19-420 | N-tolyl | N-tolyl | t-bu | b5 | H |
| 2-19-421 | N-tolyl | N-tolyl | methyl | b5 | H |
| 2-19-422 | N-tolyl | N-tolyl | b1 | b5 | H |
| 2-19-423 | N-tolyl | N-tolyl | b2 | b5 | H |
| 2-19-424 | N-tolyl | N-tolyl | b3 | b5 | H |
| 2-19-425 | N-tolyl | N-tolyl | H | b6 | H |
| 2-19-426 | N-tolyl | N-tolyl | t-bu | b6 | H |
| 2-19-427 | N-tolyl | N-tolyl | methyl | b6 | H |
| 2-19-428 | N-tolyl | N-tolyl | b1 | b6 | H |
| 2-19-429 | N-tolyl | N-tolyl | b2 | b6 | H |
| 2-19-430 | N-tolyl | N-tolyl | b3 | b6 | H |
| 2-19-431 | N-tolyl | N-tolyl | H | b7 | H |
| 2-19-432 | N-tolyl | N-tolyl | t-bu | b7 | H |
| 2-19-433 | N-tolyl | N-tolyl | methyl | b7 | H |
| 2-19-434 | N-tolyl | N-tolyl | b1 | b7 | H |
| 2-19-435 | N-tolyl | N-tolyl | b2 | b7 | H |
| 2-19-436 | N-tolyl | N-tolyl | b3 | b7 | H |
| 2-19-437 | N-tolyl | N-tolyl | H | b8 | H |
| 2-19-438 | N-tolyl | N-tolyl | t-bu | b8 | H |
| 2-19-439 | N-tolyl | N-tolyl | methyl | b8 | H |
| 2-19-440 | N-tolyl | N-tolyl | b1 | b8 | H |
| 2-19-441 | N-tolyl | N-tolyl | b2 | b8 | H |
| 2-19-442 | N-tolyl | N-tolyl | b3 | b8 | H |
| 2-19-443 | N-tolyl | N-tolyl | H | b9 | H |
| 2-19-444 | N-tolyl | N-tolyl | t-bu | b9 | H |
| 2-19-445 | N-tolyl | N-tolyl | methyl | b9 | H |
| 2-19-446 | N-tolyl | N-tolyl | b1 | b9 | H |
| 2-19-447 | N-tolyl | N-tolyl | b2 | b9 | H |
| 2-19-448 | N-tolyl | N-tolyl | b3 | b9 | H |
| 2-19-449 | N-tolyl | N-tolyl | H | b10 | H |
| 2-19-450 | N-tolyl | N-tolyl | t-bu | b10 | H |
| 2-19-451 | N-tolyl | N-tolyl | methyl | b10 | H |
| 2-19-452 | N-tolyl | N-tolyl | b1 | b10 | H |
| 2-19-453 | N-tolyl | N-tolyl | b2 | b10 | H |
| 2-19-454 | N-tolyl | N-tolyl | b3 | b10 | H |
| 2-19-455 | N-tolyl | N-tolyl | H | a1 | a1 |
| 2-19-456 | N-tolyl | N-tolyl | t-bu | a1 | a1 |
| 2-19-457 | N-tolyl | N-tolyl | methyl | a1 | a1 |
| 2-19-458 | N-tolyl | N-tolyl | b1 | a1 | a1 |
| 2-19-459 | N-tolyl | N-tolyl | b2 | a1 | a1 |
| 2-19-460 | N-tolyl | N-tolyl | b3 | a1 | a1 |
| 2-19-461 | N-tolyl | N-tolyl | H | a2 | a2 |
| 2-19-462 | N-tolyl | N-tolyl | t-bu | a2 | a2 |
| 2-19-463 | N-tolyl | N-tolyl | methyl | a2 | a2 |
| 2-19-464 | N-tolyl | N-tolyl | b1 | a2 | a2 |
| 2-19-465 | N-tolyl | N-tolyl | b2 | a2 | a2 |
| 2-19-466 | N-tolyl | N-tolyl | b3 | a2 | a2 |
| 2-19-467 | N-tolyl | N-tolyl | H | a3 | a3 |
| 2-19-468 | N-tolyl | N-tolyl | t-bu | a3 | a3 |
| 2-19-469 | N-tolyl | N-tolyl | methyl | a3 | a3 |
| 2-19-470 | N-tolyl | N-tolyl | b1 | a3 | a3 |
| 2-19-471 | N-tolyl | N-tolyl | b2 | a3 | a3 |
| 2-19-472 | N-tolyl | N-tolyl | b3 | a3 | a3 |
| 2-19-473 | N-tolyl | N-tolyl | H | b1 | b1 |
| 2-19-474 | N-tolyl | N-tolyl | t-bu | b1 | b1 |
| 2-19-475 | N-tolyl | N-tolyl | methyl | b1 | b1 |
| 2-19-476 | N-tolyl | N-tolyl | b1 | b1 | b1 |
| 2-19-477 | N-tolyl | N-tolyl | b2 | b1 | b1 |
| 2-19-478 | N-tolyl | N-tolyl | b3 | b1 | b1 |
| 2-19-479 | N-tolyl | N-tolyl | H | b2 | b2 |
| 2-19-480 | N-tolyl | N-tolyl | t-bu | b2 | b2 |
| 2-19-481 | N-tolyl | N-tolyl | methyl | b2 | b2 |
| 2-19-482 | N-tolyl | N-tolyl | b1 | b2 | b2 |
| 2-19-483 | N-tolyl | N-tolyl | b2 | b2 | b2 |
| 2-19-484 | N-tolyl | N-tolyl | b3 | b2 | b2 |
| 2-19-485 | N-tolyl | N-tolyl | H | b3 | b3 |
| 2-19-486 | N-tolyl | N-tolyl | t-bu | b3 | b3 |
| 2-19-487 | N-tolyl | N-tolyl | methyl | b3 | b3 |
| 2-19-488 | N-tolyl | N-tolyl | b1 | b3 | b3 |
| 2-19-489 | N-tolyl | N-tolyl | b2 | b3 | b3 |
| 2-19-490 | N-tolyl | N-tolyl | b3 | b3 | b3 |
| 2-19-491 | N-tolyl | N-tolyl | H | b5 | b5 |
| 2-19-492 | N-tolyl | N-tolyl | t-bu | b5 | b5 |
| 2-19-493 | N-tolyl | N-tolyl | methyl | b5 | b5 |
| 2-19-494 | N-tolyl | N-tolyl | b1 | b5 | b5 |
| 2-19-495 | N-tolyl | N-tolyl | b2 | b5 | b5 |
| 2-19-496 | N-tolyl | N-tolyl | b3 | b5 | b5 |
| 2-19-497 | N-tolyl | N-tolyl | H | b6 | b6 |
| 2-19-498 | N-tolyl | N-tolyl | t-bu | b6 | b6 |
| 2-19-499 | N-tolyl | N-tolyl | methyl | b6 | b6 |
| 2-19-500 | N-tolyl | N-tolyl | b1 | b6 | b6 |
| 2-19-501 | N-tolyl | N-tolyl | b2 | b6 | b6 |
| 2-19-502 | N-tolyl | N-tolyl | b3 | b6 | b6 |
| 2-19-503 | N-tolyl | N-tolyl | H | b7 | b7 |
| 2-19-504 | N-tolyl | N-tolyl | t-bu | b7 | b7 |
| 2-19-505 | N-tolyl | N-tolyl | methyl | b7 | b7 |
| 2-19-506 | N-tolyl | N-tolyl | b1 | b7 | b7 |
| 2-19-507 | N-tolyl | N-tolyl | b2 | b7 | b7 |
| 2-19-508 | N-tolyl | N-tolyl | b3 | b7 | b7 |
| 2-19-509 | N-Biph | N-Biph | H | H | H |
| 2-19-510 | N-Biph | N-Biph | t-bu | H | H |
| 2-19-511 | N-Biph | N-Biph | methyl | H | H |
| 2-19-512 | N-Biph | N-Biph | t-bu | t-bu | t-bu |
| 2-19-513 | N-Biph | N-Biph | methyl | methyl | methyl |
| 2-19-514 | N-Biph | N-Biph | b1 | H | H |
| 2-19-515 | N-Biph | N-Biph | b2 | H | H |
| 2-19-516 | N-Biph | N-Biph | b3 | H | H |
| 2-19-517 | N-Biph | N-Biph | H | H | a1 |
| 2-19-518 | N-Biph | N-Biph | t-bu | H | a1 |
| 2-19-519 | N-Biph | N-Biph | methyl | H | a1 |
| 2-19-520 | N-Biph | N-Biph | b1 | H | a1 |
| 2-19-521 | N-Biph | N-Biph | b2 | H | a1 |
| 2-19-522 | N-Biph | N-Biph | b3 | H | a1 |
| 2-19-523 | N-Biph | N-Biph | H | H | a2 |
| 2-19-524 | N-Biph | N-Biph | t-bu | H | a2 |
| 2-19-525 | N-Biph | N-Biph | methyl | H | a2 |
| 2-19-526 | N-Biph | N-Biph | b1 | H | a2 |
| 2-19-527 | N-Biph | N-Biph | b2 | H | a2 |
| 2-19-528 | N-Biph | N-Biph | b3 | H | a2 |
| 2-19-529 | N-Biph | N-Biph | H | H | a3 |
| 2-19-530 | N-Biph | N-Biph | t-bu | H | a3 |
| 2-19-531 | N-Biph | N-Biph | methyl | H | a3 |
| 2-19-532 | N-Biph | N-Biph | b1 | H | a3 |
| 2-19-533 | N-Biph | N-Biph | b2 | H | a3 |
| 2-19-534 | N-Biph | N-Biph | b3 | H | a3 |
| 2-19-535 | N-Biph | N-Biph | H | H | a4 |
| 2-19-536 | N-Biph | N-Biph | t-bu | H | a4 |
| 2-19-537 | N-Biph | N-Biph | methyl | H | a4 |
| 2-19-538 | N-Biph | N-Biph | b1 | H | a4 |
| 2-19-539 | N-Biph | N-Biph | b2 | H | a4 |
| 2-19-540 | N-Biph | N-Biph | b3 | H | a4 |
| 2-19-541 | N-Biph | N-Biph | H | H | a5 |
| 2-19-542 | N-Biph | N-Biph | t-bu | H | a5 |
| 2-19-543 | N-Biph | N-Biph | methyl | H | a5 |
| 2-19-544 | N-Biph | N-Biph | b1 | H | a5 |
| 2-19-545 | N-Biph | N-Biph | b2 | H | a5 |
| 2-19-546 | N-Biph | N-Biph | b3 | H | a5 |
| 2-19-547 | N-Biph | N-Biph | H | H | a6 |
| 2-19-548 | N-Biph | N-Biph | t-bu | H | a6 |
| 2-19-549 | N-Biph | N-Biph | methyl | H | a6 |
| 2-19-550 | N-Biph | N-Biph | b1 | H | a6 |
| 2-19-551 | N-Biph | N-Biph | b2 | H | a6 |
| 2-19-552 | N-Biph | N-Biph | b3 | H | a6 |
| 2-19-553 | N-Biph | N-Biph | H | H | b1 |
| 2-19-554 | N-Biph | N-Biph | t-bu | H | b1 |
| 2-19-555 | N-Biph | N-Biph | methyl | H | b1 |
| 2-19-556 | N-Biph | N-Biph | b1 | H | b1 |
| 2-19-557 | N-Biph | N-Biph | b2 | H | b1 |
| 2-19-558 | N-Biph | N-Biph | b3 | H | b1 |
| 2-19-559 | N-Biph | N-Biph | H | H | b2 |
| 2-19-560 | N-Biph | N-Biph | t-bu | H | b2 |
| 2-19-561 | N-Biph | N-Biph | methyl | H | b2 |
| 2-19-562 | N-Biph | N-Biph | b1 | H | b2 |
| 2-19-563 | N-Biph | N-Biph | b2 | H | b2 |
| 2-19-564 | N-Biph | N-Biph | b3 | H | b2 |
| 2-19-565 | N-Biph | N-Biph | H | H | b3 |
| 2-19-566 | N-Biph | N-Biph | t-bu | H | b3 |
| 2-19-567 | N-Biph | N-Biph | methyl | H | b3 |
| 2-19-568 | N-Biph | N-Biph | b1 | H | b3 |
| 2-19-569 | N-Biph | N-Biph | b2 | H | b3 |
| 2-19-570 | N-Biph | N-Biph | b3 | H | b3 |
| 2-19-571 | N-Biph | N-Biph | H | H | b4 |
| 2-19-572 | N-Biph | N-Biph | t-bu | H | b4 |
| 2-19-573 | N-Biph | N-Biph | methyl | H | b4 |
| 2-19-574 | N-Biph | N-Biph | b1 | H | b4 |
| 2-19-575 | N-Biph | N-Biph | b2 | H | b4 |
| 2-19-576 | N-Biph | N-Biph | b3 | H | b4 |
| 2-19-577 | N-Biph | N-Biph | H | H | b5 |
| 2-19-578 | N-Biph | N-Biph | t-bu | H | b5 |
| 2-19-579 | N-Biph | N-Biph | methyl | H | b5 |
| 2-19-580 | N-Biph | N-Biph | b1 | H | b5 |
| 2-19-581 | N-Biph | N-Biph | b2 | H | b5 |
| 2-19-582 | N-Biph | N-Biph | b3 | H | b5 |
| 2-19-583 | N-Biph | N-Biph | H | H | b6 |
| 2-19-584 | N-Biph | N-Biph | t-bu | H | b6 |
| 2-19-585 | N-Biph | N-Biph | methyl | H | b6 |
| 2-19-586 | N-Biph | N-Biph | b1 | H | b6 |
| 2-19-587 | N-Biph | N-Biph | b2 | H | b6 |
| 2-19-588 | N-Biph | N-Biph | b3 | H | b6 |
| 2-19-589 | N-Biph | N-Biph | H | H | b7 |
| 2-19-590 | N-Biph | N-Biph | t-bu | H | b7 |
| 2-19-591 | N-Biph | N-Biph | methyl | H | b7 |
| 2-19-592 | N-Biph | N-Biph | b1 | H | b7 |
| 2-19-593 | N-Biph | N-Biph | b2 | H | b7 |
| 2-19-594 | N-Biph | N-Biph | b3 | H | b7 |
| 2-19-595 | N-Biph | N-Biph | H | H | b8 |
| 2-19-596 | N-Biph | N-Biph | t-bu | H | b8 |
| 2-19-597 | N-Biph | N-Biph | methyl | H | b8 |
| 2-19-598 | N-Biph | N-Biph | b1 | H | b8 |
| 2-19-599 | N-Biph | N-Biph | b2 | H | b8 |
| 2-19-600 | N-Biph | N-Biph | b3 | H | b8 |
| 2-19-601 | N-Biph | N-Biph | H | H | b9 |
| 2-19-602 | N-Biph | N-Biph | t-bu | H | b9 |
| 2-19-603 | N-Biph | N-Biph | methyl | H | b9 |
| 2-19-604 | N-Biph | N-Biph | b1 | H | b9 |
| 2-19-605 | N-Biph | N-Biph | b2 | H | b9 |
| 2-19-606 | N-Biph | N-Biph | b3 | H | b9 |
| 2-19-607 | N-Biph | N-Biph | H | H | b10 |
| 2-19-608 | N-Biph | N-Biph | t-bu | H | b10 |
| 2-19-609 | N-Biph | N-Biph | methyl | H | b10 |
| 2-19-610 | N-Biph | N-Biph | b1 | H | b10 |
| 2-19-611 | N-Biph | N-Biph | b2 | H | b10 |
| 2-19-612 | N-Biph | N-Biph | b3 | H | b10 |
| 2-19-613 | N-Biph | N-Biph | H | a1 | H |
| 2-19-614 | N-Biph | N-Biph | t-bu | a1 | H |
| 2-19-615 | N-Biph | N-Biph | methyl | a1 | H |
| 2-19-616 | N-Biph | N-Biph | b1 | a1 | H |
| 2-19-617 | N-Biph | N-Biph | b2 | a1 | H |
| 2-19-618 | N-Biph | N-Biph | b3 | a1 | H |
| 2-19-619 | N-Biph | N-Biph | H | a2 | H |
| 2-19-620 | N-Biph | N-Biph | t-bu | a2 | H |
| 2-19-621 | N-Biph | N-Biph | methyl | a2 | H |
| 2-19-622 | N-Biph | N-Biph | b1 | a2 | H |
| 2-19-623 | N-Biph | N-Biph | b2 | a2 | H |
| 2-19-624 | N-Biph | N-Biph | b3 | a2 | H |
| 2-19-625 | N-Biph | N-Biph | H | a3 | H |
| 2-19-626 | N-Biph | N-Biph | t-bu | a3 | H |
| 2-19-627 | N-Biph | N-Biph | methyl | a3 | H |
| 2-19-628 | N-Biph | N-Biph | b1 | a3 | H |
| 2-19-629 | N-Biph | N-Biph | b2 | a3 | H |
| 2-19-630 | N-Biph | N-Biph | b3 | a3 | H |
| 2-19-631 | N-Biph | N-Biph | H | a4 | H |
| 2-19-632 | N-Biph | N-Biph | t-bu | a4 | H |
| 2-19-633 | N-Biph | N-Biph | methyl | a4 | H |
| 2-19-634 | N-Biph | N-Biph | b1 | a4 | H |
| 2-19-635 | N-Biph | N-Biph | b2 | a4 | H |
| 2-19-636 | N-Biph | N-Biph | b3 | a4 | H |
| 2-19-637 | N-Biph | N-Biph | H | a5 | H |
| 2-19-638 | N-Biph | N-Biph | t-bu | a5 | H |
| 2-19-639 | N-Biph | N-Biph | methyl | a5 | H |
| 2-19-640 | N-Biph | N-Biph | b1 | a5 | H |
| 2-19-641 | N-Biph | N-Biph | b2 | a5 | H |
| 2-19-642 | N-Biph | N-Biph | b3 | a5 | H |
| 2-19-643 | N-Biph | N-Biph | H | a6 | H |
| 2-19-644 | N-Biph | N-Biph | t-bu | a6 | H |
| 2-19-645 | N-Biph | N-Biph | methyl | a6 | H |
| 2-19-646 | N-Biph | N-Biph | b1 | a6 | H |
| 2-19-647 | N-Biph | N-Biph | b2 | a6 | H |
| 2-19-648 | N-Biph | N-Biph | b3 | a6 | H |
| 2-19-649 | N-Biph | N-Biph | H | b1 | H |
| 2-19-650 | N-Biph | N-Biph | t-bu | b1 | H |
| 2-19-651 | N-Biph | N-Biph | methyl | b1 | H |
| 2-19-652 | N-Biph | N-Biph | b1 | b1 | H |
| 2-19-653 | N-Biph | N-Biph | b2 | b1 | H |
| 2-19-654 | N-Biph | N-Biph | b3 | b1 | H |
| 2-19-655 | N-Biph | N-Biph | H | b2 | H |
| 2-19-656 | N-Biph | N-Biph | t-bu | b2 | H |
| 2-19-657 | N-Biph | N-Biph | methyl | b2 | H |
| 2-19-658 | N-Biph | N-Biph | b1 | b2 | H |
| 2-19-659 | N-Biph | N-Biph | b2 | b2 | H |
| 2-19-660 | N-Biph | N-Biph | b3 | b2 | H |
| 2-19-661 | N-Biph | N-Biph | H | b3 | H |
| 2-19-662 | N-Biph | N-Biph | t-bu | b3 | H |
| 2-19-663 | N-Biph | N-Biph | methyl | b3 | H |
| 2-19-664 | N-Biph | N-Biph | b1 | b3 | H |
| 2-19-665 | N-Biph | N-Biph | b2 | b3 | H |
| 2-19-666 | N-Biph | N-Biph | b3 | b3 | H |
| 2-19-667 | N-Biph | N-Biph | H | b4 | H |
| 2-19-668 | N-Biph | N-Biph | t-bu | b4 | H |
| 2-19-669 | N-Biph | N-Biph | methyl | b4 | H |
| 2-19-670 | N-Biph | N-Biph | b1 | b4 | H |
| 2-19-671 | N-Biph | N-Biph | b2 | b4 | H |
| 2-19-672 | N-Biph | N-Biph | b3 | b4 | H |
| 2-19-673 | N-Biph | N-Biph | H | b5 | H |
| 2-19-674 | N-Biph | N-Biph | t-bu | b5 | H |
| 2-19-675 | N-Biph | N-Biph | methyl | b5 | H |
| 2-19-676 | N-Biph | N-Biph | b1 | b5 | H |
| 2-19-677 | N-Biph | N-Biph | b2 | b5 | H |
| 2-19-678 | N-Biph | N-Biph | b3 | b5 | H |
| 2-19-679 | N-Biph | N-Biph | H | b6 | H |
| 2-19-680 | N-Biph | N-Biph | t-bu | b6 | H |
| 2-19-681 | N-Biph | N-Biph | methyl | b6 | H |
| 2-19-682 | N-Biph | N-Biph | b1 | b6 | H |
| 2-19-683 | N-Biph | N-Biph | b2 | b6 | H |
| 2-19-684 | N-Biph | N-Biph | b3 | b6 | H |
| 2-19-685 | N-Biph | N-Biph | H | b7 | H |
| 2-19-686 | N-Biph | N-Biph | t-bu | b7 | H |
| 2-19-687 | N-Biph | N-Biph | methyl | b7 | H |
| 2-19-688 | N-Biph | N-Biph | b1 | b7 | H |
| 2-19-689 | N-Biph | N-Biph | b2 | b7 | H |
| 2-19-690 | N-Biph | N-Biph | b3 | b7 | H |
| 2-19-691 | N-Biph | N-Biph | H | b8 | H |
| 2-19-692 | N-Biph | N-Biph | t-bu | b8 | H |
| 2-19-693 | N-Biph | N-Biph | methyl | b8 | H |
| 2-19-694 | N-Biph | N-Biph | b1 | b8 | H |
| 2-19-695 | N-Biph | N-Biph | b2 | b8 | H |
| 2-19-696 | N-Biph | N-Biph | b3 | b8 | H |
| 2-19-697 | N-Biph | N-Biph | H | b9 | H |
| 2-19-698 | N-Biph | N-Biph | t-bu | b9 | H |
| 2-19-699 | N-Biph | N-Biph | methyl | b9 | H |
| 2-19-700 | N-Biph | N-Biph | b1 | b9 | H |
| 2-19-701 | N-Biph | N-Biph | b2 | b9 | H |
| 2-19-702 | N-Biph | N-Biph | b3 | b9 | H |
| 2-19-703 | N-Biph | N-Biph | H | b10 | H |
| 2-19-704 | N-Biph | N-Biph | t-bu | b10 | H |
| 2-19-705 | N-Biph | N-Biph | methyl | b10 | H |
| 2-19-706 | N-Biph | N-Biph | b1 | b10 | H |
| 2-19-707 | N-Biph | N-Biph | b2 | b10 | H |
| 2-19-708 | N-Biph | N-Biph | b3 | b10 | H |
| 2-19-709 | N-Biph | N-Biph | H | a1 | a1 |
| 2-19-710 | N-Biph | N-Biph | t-bu | a1 | a1 |
| 2-19-711 | N-Biph | N-Biph | methyl | a1 | a1 |
| 2-19-712 | N-Biph | N-Biph | b1 | a1 | a1 |
| 2-19-713 | N-Biph | N-Biph | b2 | a1 | a1 |
| 2-19-714 | N-Biph | N-Biph | b3 | a1 | a1 |
| 2-19-715 | N-Biph | N-Biph | H | a2 | a2 |
| 2-19-716 | N-Biph | N-Biph | t-bu | a2 | a2 |
| 2-19-717 | N-Biph | N-Biph | methyl | a2 | a2 |
| 2-19-718 | N-Biph | N-Biph | b1 | a2 | a2 |
| 2-19-719 | N-Biph | N-Biph | b2 | a2 | a2 |
| 2-19-720 | N-Biph | N-Biph | b3 | a2 | a2 |
| 2-19-721 | N-Biph | N-Biph | H | a3 | a3 |
| 2-19-722 | N-Biph | N-Biph | t-bu | a3 | a3 |
| 2-19-723 | N-Biph | N-Biph | methyl | a3 | a3 |
| 2-19-724 | N-Biph | N-Biph | b1 | a3 | a3 |
| 2-19-725 | N-Biph | N-Biph | b2 | a3 | a3 |
| 2-19-726 | N-Biph | N-Biph | b3 | a3 | a3 |
| 2-19-727 | N-Biph | N-Biph | H | b1 | b1 |
| 2-19-728 | N-Biph | N-Biph | t-bu | b1 | b1 |
| 2-19-729 | N-Biph | N-Biph | methyl | b1 | b1 |
| 2-19-730 | N-Biph | N-Biph | b1 | b1 | b1 |
| 2-19-731 | N-Biph | N-Biph | b2 | b1 | b1 |
| 2-19-732 | N-Biph | N-Biph | b3 | b1 | b1 |
| 2-19-733 | N-Biph | N-Biph | H | b2 | b2 |
| 2-19-734 | N-Biph | N-Biph | t-bu | b2 | b2 |
| 2-19-735 | N-Biph | N-Biph | methyl | b2 | b2 |
| 2-19-736 | N-Biph | N-Biph | b1 | b2 | b2 |
| 2-19-737 | N-Biph | N-Biph | b2 | b2 | b2 |
| 2-19-738 | N-Biph | N-Biph | b3 | b2 | b2 |
| 2-19-739 | N-Biph | N-Biph | H | b3 | b3 |
| 2-19-740 | N-Biph | N-Biph | t-bu | b3 | b3 |
| 2-19-741 | N-Biph | N-Biph | methyl | b3 | b3 |
| 2-19-742 | N-Biph | N-Biph | b1 | b3 | b3 |
| 2-19-743 | N-Biph | N-Biph | b2 | b3 | b3 |
| 2-19-744 | N-Biph | N-Biph | b3 | b3 | b3 |
| 2-19-745 | N-Biph | N-Biph | H | b5 | b5 |
| 2-19-746 | N-Biph | N-Biph | t-bu | b5 | b5 |
| 2-19-747 | N-Biph | N-Biph | methyl | b5 | b5 |
| 2-19-748 | N-Biph | N-Biph | b1 | b5 | b5 |
| 2-19-749 | N-Biph | N-Biph | b2 | b5 | b5 |
| 2-19-750 | N-Biph | N-Biph | b3 | b5 | b5 |
| 2-19-751 | N-Biph | N-Biph | H | b6 | b6 |
| 2-19-752 | N-Biph | N-Biph | t-bu | b6 | b6 |
| 2-19-753 | N-Biph | N-Biph | methyl | b6 | b6 |
| 2-19-754 | N-Biph | N-Biph | b1 | b6 | b6 |
| 2-19-755 | N-Biph | N-Biph | b2 | b6 | b6 |
| 2-19-756 | N-Biph | N-Biph | b3 | b6 | b6 |
| 2-19-757 | N-Biph | N-Biph | H | b7 | b7 |
| 2-19-758 | N-Biph | N-Biph | t-bu | b7 | b7 |
| 2-19-759 | N-Biph | N-Biph | methyl | b7 | b7 |
| 2-19-760 | N-Biph | N-Biph | b1 | b7 | b7 |
| 2-19-761 | N-Biph | N-Biph | b2 | b7 | b7 |
| 2-19-762 | N-Biph | N-Biph | b3 | b7 | b7 |
| 2-19-763 | O | N Ph | H | H | H |
| 2-19-764 | O | N Ph | t-bu | H | H |
| 2-19-765 | O | N Ph | methyl | H | H |
| 2-19-766 | O | N Ph | t-bu | t-bu | t-bu |
| 2-19-767 | O | N Ph | methyl | methyl | methyl |
| 2-19-768 | O | N Ph | b1 | H | H |
| 2-19-769 | O | N Ph | b2 | H | H |
| 2-19-770 | O | N Ph | b3 | H | H |
| 2-19-771 | O | N Ph | H | H | a1 |
| 2-19-772 | O | N Ph | t-bu | H | a1 |
| 2-19-773 | O | N Ph | methyl | H | a1 |
| 2-19-774 | O | N Ph | b1 | H | a1 |
| 2-19-775 | O | N Ph | b2 | H | a1 |
| 2-19-776 | O | N Ph | b3 | H | a1 |
| 2-19-777 | O | N Ph | H | H | a2 |
| 2-19-778 | O | N Ph | t-bu | H | a2 |
| 2-19-779 | O | N Ph | methyl | H | a2 |
| 2-19-780 | O | N Ph | b1 | H | a2 |
| 2-19-781 | O | N Ph | b2 | H | a2 |
| 2-19-782 | O | N Ph | b3 | H | a2 |
| 2-19-783 | O | N Ph | H | H | a3 |
| 2-19-784 | O | N Ph | t-bu | H | a3 |
| 2-19-785 | O | N Ph | methyl | H | a3 |
| 2-19-786 | O | N Ph | b1 | H | a3 |
| 2-19-787 | O | N Ph | b2 | H | a3 |
| 2-19-788 | O | N Ph | b3 | H | a3 |
| 2-19-789 | O | N Ph | H | H | a4 |
| 2-19-790 | O | N Ph | t-bu | H | a4 |
| 2-19-791 | O | N Ph | methyl | H | a4 |
| 2-19-792 | O | N Ph | b1 | H | a4 |
| 2-19-793 | O | N Ph | b2 | H | a4 |
| 2-19-794 | O | N Ph | b3 | H | a4 |
| 2-19-795 | O | N Ph | H | H | a5 |
| 2-19-796 | O | N Ph | t-bu | H | a5 |
| 2-19-797 | O | N Ph | methyl | H | a5 |
| 2-19-798 | O | N Ph | b1 | H | a5 |
| 2-19-799 | O | N Ph | b2 | H | a5 |
| 2-19-800 | O | N Ph | b3 | H | a5 |
| 2-19-801 | O | N Ph | H | H | a6 |
| 2-19-802 | O | N Ph | t-bu | H | a6 |
| 2-19-803 | O | N Ph | methyl | H | a6 |
| 2-19-804 | O | N Ph | b1 | H | a6 |
| 2-19-805 | O | N Ph | b2 | H | a6 |
| 2-19-806 | O | N Ph | b3 | H | a6 |
| 2-19-807 | O | N Ph | H | H | b1 |
| 2-19-808 | O | N Ph | t-bu | H | b1 |
| 2-19-809 | O | N Ph | methyl | H | b1 |
| 2-19-810 | O | N Ph | b1 | H | b1 |
| 2-19-811 | O | N Ph | b2 | H | b1 |
| 2-19-812 | O | N Ph | b3 | H | b1 |
| 2-19-813 | O | N Ph | H | H | b2 |
| 2-19-814 | O | N Ph | t-bu | H | b2 |
| 2-19-815 | O | N Ph | methyl | H | b2 |
| 2-19-816 | O | N Ph | b1 | H | b2 |
| 2-19-817 | O | N Ph | b2 | H | b2 |
| 2-19-818 | O | N Ph | b3 | H | b2 |
| 2-19-819 | O | N Ph | H | H | b3 |
| 2-19-820 | O | N Ph | t-bu | H | b3 |
| 2-19-821 | O | N Ph | methyl | H | b3 |
| 2-19-822 | O | N Ph | b1 | H | b3 |
| 2-19-823 | O | N Ph | b2 | H | b3 |
| 2-19-824 | O | N Ph | b3 | H | b3 |
| 2-19-825 | O | N Ph | H | H | b4 |
| 2-19-826 | O | N Ph | t-bu | H | b4 |
| 2-19-827 | O | N Ph | methyl | H | b4 |
| 2-19-828 | O | N Ph | b1 | H | b4 |
| 2-19-829 | O | N Ph | b2 | H | b4 |
| 2-19-830 | O | N Ph | b3 | H | b4 |
| 2-19-831 | O | N Ph | H | H | b5 |
| 2-19-832 | O | N Ph | t-bu | H | b5 |
| 2-19-833 | O | N Ph | methyl | H | b5 |
| 2-19-834 | O | N Ph | b1 | H | b5 |
| 2-19-835 | O | N Ph | b2 | H | b5 |
| 2-19-836 | O | N Ph | b3 | H | b5 |
| 2-19-837 | O | N Ph | H | H | b6 |
| 2-19-838 | O | N Ph | t-bu | H | b6 |
| 2-19-839 | O | N Ph | methyl | H | b6 |
| 2-19-840 | O | N Ph | b1 | H | b6 |
| 2-19-841 | O | N Ph | b2 | H | b6 |
| 2-19-842 | O | N Ph | b3 | H | b6 |
| 2-19-843 | O | N Ph | H | H | b7 |
| 2-19-844 | O | N Ph | t-bu | H | b7 |
| 2-19-845 | O | N Ph | methyl | H | b7 |
| 2-19-846 | O | N Ph | b1 | H | b7 |
| 2-19-847 | O | N Ph | b2 | H | b7 |
| 2-19-848 | O | N Ph | b3 | H | b7 |
| 2-19-849 | O | N Ph | H | H | b8 |
| 2-19-850 | O | N Ph | t-bu | H | b8 |
| 2-19-851 | O | N Ph | methyl | H | b8 |
| 2-19-852 | O | N Ph | b1 | H | b8 |
| 2-19-853 | O | N Ph | b2 | H | b8 |
| 2-19-854 | O | N Ph | b3 | H | b8 |
| 2-19-855 | O | N Ph | H | H | b9 |
| 2-19-856 | O | N Ph | t-bu | H | b9 |
| 2-19-857 | O | N Ph | methyl | H | b9 |
| 2-19-858 | O | N Ph | b1 | H | b9 |
| 2-19-859 | O | N Ph | b2 | H | b9 |
| 2-19-860 | O | N Ph | b3 | H | b9 |
| 2-19-861 | O | N Ph | H | H | b10 |
| 2-19-862 | O | N Ph | t-bu | H | b10 |
| 2-19-863 | O | N Ph | methyl | H | b10 |
| 2-19-864 | O | N Ph | b1 | H | b10 |
| 2-19-865 | O | N Ph | b2 | H | b10 |
| 2-19-866 | O | N Ph | b3 | H | b10 |
| 2-19-867 | O | N Ph | H | a1 | H |
| 2-19-868 | O | N Ph | t-bu | a1 | H |
| 2-19-869 | O | N Ph | methyl | a1 | H |
| 2-19-870 | O | N Ph | b1 | a1 | H |
| 2-19-871 | O | N Ph | b2 | a1 | H |
| 2-19-872 | O | N Ph | b3 | a1 | H |
| 2-19-873 | O | N Ph | H | a2 | H |
| 2-19-874 | O | N Ph | t-bu | a2 | H |
| 2-19-875 | O | N Ph | methyl | a2 | H |
| 2-19-876 | O | N Ph | b1 | a2 | H |
| 2-19-877 | O | N Ph | b2 | a2 | H |
| 2-19-878 | O | N Ph | b3 | a2 | H |
| 2-19-879 | O | N Ph | H | a3 | H |
| 2-19-880 | O | N Ph | t-bu | a3 | H |
| 2-19-881 | O | N Ph | methyl | a3 | H |
| 2-19-882 | O | N Ph | b1 | a3 | H |
| 2-19-883 | O | N Ph | b2 | a3 | H |
| 2-19-884 | O | N Ph | b3 | a3 | H |
| 2-19-885 | O | N Ph | H | a4 | H |
| 2-19-886 | O | N Ph | t-bu | a4 | H |
| 2-19-887 | O | N Ph | methyl | a4 | H |
| 2-19-888 | O | N Ph | b1 | a4 | H |
| 2-19-889 | O | N Ph | b2 | a4 | H |
| 2-19-890 | O | N Ph | b3 | a4 | H |
| 2-19-891 | O | N Ph | H | a5 | H |
| 2-19-892 | O | N Ph | t-bu | a5 | H |
| 2-19-893 | O | N Ph | methyl | a5 | H |
| 2-19-894 | O | N Ph | b1 | a5 | H |
| 2-19-895 | O | N Ph | b2 | a5 | H |
| 2-19-896 | O | N Ph | b3 | a5 | H |
| 2-19-897 | O | N Ph | H | a6 | H |
| 2-19-898 | O | N Ph | t-bu | a6 | H |
| 2-19-899 | O | N Ph | methyl | a6 | H |
| 2-19-900 | O | N Ph | b1 | a6 | H |
| 2-19-901 | O | N Ph | b2 | a6 | H |
| 2-19-902 | O | N Ph | b3 | a6 | H |
| 2-19-903 | O | N Ph | H | b1 | H |
| 2-19-904 | O | N Ph | t-bu | b1 | H |
| 2-19-905 | O | N Ph | methyl | b1 | H |
| 2-19-906 | O | N Ph | b1 | b1 | H |
| 2-19-907 | O | N Ph | b2 | b1 | H |
| 2-19-908 | O | N Ph | b3 | b1 | H |
| 2-19-909 | O | N Ph | H | b2 | H |
| 2-19-910 | O | N Ph | t-bu | b2 | H |
| 2-19-911 | O | N Ph | methyl | b2 | H |
| 2-19-912 | O | N Ph | b1 | b2 | H |
| 2-19-913 | O | N Ph | b2 | b2 | H |
| 2-19-914 | O | N Ph | b3 | b2 | H |
| 2-19-915 | O | N Ph | H | b3 | H |
| 2-19-916 | O | N Ph | t-bu | b3 | H |
| 2-19-917 | O | N Ph | methyl | b3 | H |
| 2-19-918 | O | N Ph | b1 | b3 | H |
| 2-19-919 | O | N Ph | b2 | b3 | H |
| 2-19-920 | O | N Ph | b3 | b3 | H |
| 2-19-921 | O | N Ph | H | b4 | H |
| 2-19-922 | O | N Ph | t-bu | b4 | H |
| 2-19-923 | O | N Ph | methyl | b4 | H |
| 2-19-924 | O | N Ph | b1 | b4 | H |
| 2-19-925 | O | N Ph | b2 | b4 | H |
| 2-19-926 | O | N Ph | b3 | b4 | H |
| 2-19-927 | O | N Ph | H | b5 | H |
| 2-19-928 | O | N Ph | t-bu | b5 | H |
| 2-19-929 | O | N Ph | methyl | b5 | H |
| 2-19-930 | O | N Ph | b1 | b5 | H |
| 2-19-931 | O | N Ph | b2 | b5 | H |
| 2-19-932 | O | N Ph | b3 | b5 | H |
| 2-19-933 | O | N Ph | H | b6 | H |
| 2-19-934 | O | N Ph | t-bu | b6 | H |
| 2-19-935 | O | N Ph | methyl | b6 | H |
| 2-19-936 | O | N Ph | b1 | b6 | H |
| 2-19-937 | O | N Ph | b2 | b6 | H |
| 2-19-938 | O | N Ph | b3 | b6 | H |
| 2-19-939 | O | N Ph | H | b7 | H |
| 2-19-940 | O | N Ph | t-bu | b7 | H |
| 2-19-941 | O | N Ph | methyl | b7 | H |
| 2-19-942 | O | N Ph | b1 | b7 | H |
| 2-19-943 | O | N Ph | b2 | b7 | H |
| 2-19-944 | O | N Ph | b3 | b7 | H |
| 2-19-945 | O | N Ph | H | b8 | H |
| 2-19-946 | O | N Ph | t-bu | b8 | H |
| 2-19-947 | O | N Ph | methyl | b8 | H |
| 2-19-948 | O | N Ph | b1 | b8 | H |
| 2-19-949 | O | N Ph | b2 | b8 | H |
| 2-19-950 | O | N Ph | b3 | b8 | H |
| 2-19-951 | O | N Ph | H | b9 | H |
| 2-19-952 | O | N Ph | t-bu | b9 | H |
| 2-19-953 | O | N Ph | methyl | b9 | H |
| 2-19-954 | O | N Ph | b1 | b9 | H |
| 2-19-955 | O | N Ph | b2 | b9 | H |
| 2-19-956 | O | N Ph | b3 | b9 | H |
| 2-19-957 | O | N Ph | H | b10 | H |
| 2-19-958 | O | N Ph | t-bu | b10 | H |
| 2-19-959 | O | N Ph | methyl | b10 | H |
| 2-19-960 | O | N Ph | b1 | b10 | H |
| 2-19-961 | O | N Ph | b2 | b10 | H |
| 2-19-962 | O | N Ph | b3 | b10 | H |
| 2-19-963 | O | N Ph | H | a1 | a1 |
| 2-19-964 | O | N Ph | t-bu | a1 | a1 |
| 2-19-965 | O | N Ph | methyl | a1 | a1 |
| 2-19-966 | O | N Ph | b1 | a1 | a1 |
| 2-19-967 | O | N Ph | b2 | a1 | a1 |
| 2-19-968 | O | N Ph | b3 | a1 | a1 |
| 2-19-969 | O | N Ph | H | a2 | a2 |
| 2-19-970 | O | N Ph | t-bu | a2 | a2 |
| 2-19-971 | O | N Ph | methyl | a2 | a2 |
| 2-19-972 | O | N Ph | b1 | a2 | a2 |
| 2-19-973 | O | N Ph | b2 | a2 | a2 |
| 2-19-974 | O | N Ph | b3 | a2 | a2 |
| 2-19-975 | O | N Ph | H | a3 | a3 |
| 2-19-976 | O | N Ph | t-bu | a3 | a3 |
| 2-19-977 | O | N Ph | methyl | a3 | a3 |
| 2-19-978 | O | N Ph | b1 | a3 | a3 |
| 2-19-979 | O | N Ph | b2 | a3 | a3 |
| 2-19-980 | O | N Ph | b3 | a3 | a3 |
| 2-19-981 | O | N Ph | H | b1 | b1 |
| 2-19-982 | O | N Ph | t-bu | b1 | b1 |
| 2-19-983 | O | N Ph | methyl | b1 | b1 |
| 2-19-984 | O | N Ph | b1 | b1 | b1 |
| 2-19-985 | O | N Ph | b2 | b1 | b1 |
| 2-19-986 | O | N Ph | b3 | b1 | b1 |
| 2-19-987 | O | N Ph | H | b2 | b2 |
| 2-19-988 | O | N Ph | t-bu | b2 | b2 |
| 2-19-989 | O | N Ph | methyl | b2 | b2 |
| 2-19-990 | O | N Ph | b1 | b2 | b2 |
| 2-19-991 | O | N Ph | b2 | b2 | b2 |
| 2-19-992 | O | N Ph | b3 | b2 | b2 |
| 2-19-993 | O | N Ph | H | b3 | b3 |
| 2-19-994 | O | N Ph | t-bu | b3 | b3 |
| 2-19-995 | O | N Ph | methyl | b3 | b3 |
| 2-19-996 | O | N Ph | b1 | b3 | b3 |
| 2-19-997 | O | N Ph | b2 | b3 | b3 |
| 2-19-998 | O | N Ph | b3 | b3 | b3 |
| 2-19-999 | O | N Ph | H | b5 | b5 |
| 2-19-1000 | O | N Ph | t-bu | b5 | b5 |
| 2-19-1001 | O | N Ph | methyl | b5 | b5 |
| 2-19-1002 | O | N Ph | b1 | b5 | b5 |
| 2-19-1003 | O | N Ph | b2 | b5 | b5 |
| 2-19-1004 | O | N Ph | b3 | b5 | b5 |
| 2-19-1005 | O | N Ph | H | b6 | b6 |
| 2-19-1006 | O | N Ph | t-bu | b6 | b6 |
| 2-19-1007 | O | N Ph | methyl | b6 | b6 |
| 2-19-1008 | O | N Ph | b1 | b6 | b6 |
| 2-19-1009 | O | N Ph | b2 | b6 | b6 |
| 2-19-1010 | O | N Ph | b3 | b6 | b6 |
| 2-19-1011 | O | N Ph | H | b7 | b7 |
| 2-19-1012 | O | N Ph | t-bu | b7 | b7 |
| 2-19-1013 | O | N Ph | methyl | b7 | b7 |
| 2-19-1014 | O | N Ph | b1 | b7 | b7 |
| 2-19-1015 | O | N Ph | b2 | b7 | b7 |
| 2-19-1016 | O | N Ph | b3 | b7 | b7 |
| 2-19-1017 | S | N Ph | H | H | H |
| 2-19-1018 | S | N Ph | t-bu | H | H |
| 2-19-1019 | S | N Ph | methyl | H | H |
| 2-19-1020 | S | N Ph | t-bu | t-bu | t-bu |
| 2-19-1021 | S | N Ph | methyl | methyl | methyl |
| 2-19-1022 | S | N Ph | b1 | H | H |
| 2-19-1023 | S | N Ph | b2 | H | H |
| 2-19-1024 | S | N Ph | b3 | H | H |
| 2-19-1025 | S | N Ph | H | H | a1 |
| 2-19-1026 | S | N Ph | t-bu | H | a1 |
| 2-19-1027 | S | N Ph | methyl | H | a1 |
| 2-19-1028 | S | N Ph | b1 | H | a1 |
| 2-19-1029 | S | N Ph | b2 | H | a1 |
| 2-19-1030 | S | N Ph | b3 | H | a1 |
| 2-19-1031 | S | N Ph | H | H | a2 |
| 2-19-1032 | S | N Ph | t-bu | H | a2 |
| 2-19-1033 | S | N Ph | methyl | H | a2 |
| 2-19-1034 | S | N Ph | b1 | H | a2 |
| 2-19-1035 | S | N Ph | b2 | H | a2 |
| 2-19-1036 | S | N Ph | b3 | H | a2 |
| 2-19-1037 | S | N Ph | H | H | a3 |
| 2-19-1038 | S | N Ph | t-bu | H | a3 |
| 2-19-1039 | S | N Ph | methyl | H | a3 |
| 2-19-1040 | S | N Ph | b1 | H | a3 |
| 2-19-1041 | S | N Ph | b2 | H | a3 |
| 2-19-1042 | S | N Ph | b3 | H | a3 |
| 2-19-1043 | S | N Ph | H | H | a4 |
| 2-19-1044 | S | N Ph | t-bu | H | a4 |
| 2-19-1045 | S | N Ph | methyl | H | a4 |
| 2-19-1046 | S | N Ph | b1 | H | a4 |
| 2-19-1047 | S | N Ph | b2 | H | a4 |
| 2-19-1048 | S | N Ph | b3 | H | a4 |
| 2-19-1049 | S | N Ph | H | H | a5 |
| 2-19-1050 | S | N Ph | t-bu | H | a5 |
| 2-19-1051 | S | N Ph | methyl | H | a5 |
| 2-19-1052 | S | N Ph | b1 | H | a5 |
| 2-19-1053 | S | N Ph | b2 | H | a5 |
| 2-19-1054 | S | N Ph | b3 | H | a5 |
| 2-19-1055 | S | N Ph | H | H | a6 |
| 2-19-1056 | S | N Ph | t-bu | H | a6 |
| 2-19-1057 | S | N Ph | methyl | H | a6 |
| 2-19-1058 | S | N Ph | b1 | H | a6 |
| 2-19-1059 | S | N Ph | b2 | H | a6 |
| 2-19-1060 | S | N Ph | b3 | H | a6 |
| 2-19-1061 | S | N Ph | H | H | b1 |
| 2-19-1062 | S | N Ph | t-bu | H | b1 |
| 2-19-1063 | S | N Ph | methyl | H | b1 |
| 2-19-1064 | S | N Ph | b1 | H | b1 |
| 2-19-1065 | S | N Ph | b2 | H | b1 |
| 2-19-1066 | S | N Ph | b3 | H | b1 |
| 2-19-1067 | S | N Ph | H | H | b2 |
| 2-19-1068 | S | N Ph | t-bu | H | b2 |
| 2-19-1069 | S | N Ph | methyl | H | b2 |
| 2-19-1070 | S | N Ph | b1 | H | b2 |
| 2-19-1071 | S | N Ph | b2 | H | b2 |
| 2-19-1072 | S | N Ph | b3 | H | b2 |
| 2-19-1073 | S | N Ph | H | H | b3 |
| 2-19-1074 | S | N Ph | t-bu | H | b3 |
| 2-19-1075 | S | N Ph | methyl | H | b3 |
| 2-19-1076 | S | N Ph | b1 | H | b3 |
| 2-19-1077 | S | N Ph | b2 | H | b3 |
| 2-19-1078 | S | N Ph | b3 | H | b3 |
| 2-19-1079 | S | N Ph | H | H | b4 |
| 2-19-1080 | S | N Ph | t-bu | H | b4 |
| 2-19-1081 | S | N Ph | methyl | H | b4 |
| 2-19-1082 | S | N Ph | b1 | H | b4 |
| 2-19-1083 | S | N Ph | b2 | H | b4 |
| 2-19-1084 | S | N Ph | b3 | H | b4 |
| 2-19-1085 | S | N Ph | H | H | b5 |
| 2-19-1086 | S | N Ph | t-bu | H | b5 |
| 2-19-1087 | S | N Ph | methyl | H | b5 |
| 2-19-1088 | S | N Ph | b1 | H | b5 |
| 2-19-1089 | S | N Ph | b2 | H | b5 |
| 2-19-1090 | S | N Ph | b3 | H | b5 |
| 2-19-1091 | S | N Ph | H | H | b6 |
| 2-19-1092 | S | N Ph | t-bu | H | b6 |
| 2-19-1093 | S | N Ph | methyl | H | b6 |
| 2-19-1094 | S | N Ph | b1 | H | b6 |
| 2-19-1095 | S | N Ph | b2 | H | b6 |
| 2-19-1096 | S | N Ph | b3 | H | b6 |
| 2-19-1097 | S | N Ph | H | H | b7 |
| 2-19-1098 | S | N Ph | t-bu | H | b7 |
| 2-19-1099 | S | N Ph | methyl | H | b7 |
| 2-19-1100 | S | N Ph | b1 | H | b7 |
| 2-19-1101 | S | N Ph | b2 | H | b7 |
| 2-19-1102 | S | N Ph | b3 | H | b7 |
| 2-19-1103 | S | N Ph | H | H | b8 |
| 2-19-1104 | S | N Ph | t-bu | H | b8 |
| 2-19-1105 | S | N Ph | methyl | H | b8 |
| 2-19-1106 | S | N Ph | b1 | H | b8 |
| 2-19-1107 | S | N Ph | b2 | H | b8 |
| 2-19-1108 | S | N Ph | b3 | H | b8 |
| 2-19-1109 | S | N Ph | H | H | b9 |
| 2-19-1110 | S | N Ph | t-bu | H | b9 |
| 2-19-1111 | S | N Ph | methyl | H | b9 |
| 2-19-1112 | S | N Ph | b1 | H | b9 |
| 2-19-1113 | S | N Ph | b2 | H | b9 |
| 2-19-1114 | S | N Ph | b3 | H | b9 |
| 2-19-1115 | S | N Ph | H | H | b10 |
| 2-19-1116 | S | N Ph | t-bu | H | b10 |
| 2-19-1117 | S | N Ph | methyl | H | b10 |
| 2-19-1118 | S | N Ph | b1 | H | b10 |
| 2-19-1119 | S | N Ph | b2 | H | b10 |
| 2-19-1120 | S | N Ph | b3 | H | b10 |
| 2-19-1121 | S | N Ph | H | a1 | H |
| 2-19-1122 | S | N Ph | t-bu | a1 | H |
| 2-19-1123 | S | N Ph | methyl | a1 | H |
| 2-19-1124 | S | N Ph | b1 | a1 | H |
| 2-19-1125 | S | N Ph | b2 | a1 | H |
| 2-19-1126 | S | N Ph | b3 | a1 | H |
| 2-19-1127 | S | N Ph | H | a2 | H |
| 2-19-1128 | S | N Ph | t-bu | a2 | H |
| 2-19-1129 | S | N Ph | methyl | a2 | H |
| 2-19-1130 | S | N Ph | b1 | a2 | H |
| 2-19-1131 | S | N Ph | b2 | a2 | H |
| 2-19-1132 | S | N Ph | b3 | a2 | H |
| 2-19-1133 | S | N Ph | H | a3 | H |
| 2-19-1134 | S | N Ph | t-bu | a3 | H |
| 2-19-1135 | S | N Ph | methyl | a3 | H |
| 2-19-1136 | S | N Ph | b1 | a3 | H |
| 2-19-1137 | S | N Ph | b2 | a3 | H |
| 2-19-1138 | S | N Ph | b3 | a3 | H |
| 2-19-1139 | S | N Ph | H | a4 | H |
| 2-19-1140 | S | N Ph | t-bu | a4 | H |
| 2-19-1141 | S | N Ph | methyl | a4 | H |
| 2-19-1142 | S | N Ph | b1 | a4 | H |
| 2-19-1143 | S | N Ph | b2 | a4 | H |
| 2-19-1144 | S | N Ph | b3 | a4 | H |
| 2-19-1145 | S | N Ph | H | a5 | H |
| 2-19-1146 | S | N Ph | t-bu | a5 | H |
| 2-19-1147 | S | N Ph | methyl | a5 | H |
| 2-19-1148 | S | N Ph | b1 | a5 | H |
| 2-19-1149 | S | N Ph | b2 | a5 | H |
| 2-19-1150 | S | N Ph | b3 | a5 | H |
| 2-19-1151 | S | N Ph | H | a6 | H |
| 2-19-1152 | S | N Ph | t-bu | a6 | H |
| 2-19-1153 | S | N Ph | methyl | a6 | H |
| 2-19-1154 | S | N Ph | b1 | a6 | H |
| 2-19-1155 | S | N Ph | b2 | a6 | H |
| 2-19-1156 | S | N Ph | b3 | a6 | H |
| 2-19-1157 | S | N Ph | H | b1 | H |
| 2-19-1158 | S | N Ph | t-bu | b1 | H |
| 2-19-1159 | S | N Ph | methyl | b1 | H |
| 2-19-1160 | S | N Ph | b1 | b1 | H |
| 2-19-1161 | S | N Ph | b2 | b1 | H |
| 2-19-1162 | S | N Ph | b3 | b1 | H |
| 2-19-1163 | S | N Ph | H | b2 | H |
| 2-19-1164 | S | N Ph | t-bu | b2 | H |
| 2-19-1165 | S | N Ph | methyl | b2 | H |
| 2-19-1166 | S | N Ph | b1 | b2 | H |
| 2-19-1167 | S | N Ph | b2 | b2 | H |
| 2-19-1168 | S | N Ph | b3 | b2 | H |
| 2-19-1169 | S | N Ph | H | b3 | H |
| 2-19-1170 | S | N Ph | t-bu | b3 | H |
| 2-19-1171 | S | N Ph | methyl | b3 | H |
| 2-19-1172 | S | N Ph | b1 | b3 | H |
| 2-19-1173 | S | N Ph | b2 | b3 | H |
| 2-19-1174 | S | N Ph | b3 | b3 | H |
| 2-19-1175 | S | N Ph | H | b4 | H |
| 2-19-1176 | S | N Ph | t-bu | b4 | H |
| 2-19-1177 | S | N Ph | methyl | b4 | H |
| 2-19-1178 | S | N Ph | b1 | b4 | H |
| 2-19-1179 | S | N Ph | b2 | b4 | H |
| 2-19-1180 | S | N Ph | b3 | b4 | H |
| 2-19-1181 | S | N Ph | H | b5 | H |
| 2-19-1182 | S | N Ph | t-bu | b5 | H |
| 2-19-1183 | S | N Ph | methyl | b5 | H |
| 2-19-1184 | S | N Ph | b1 | b5 | H |
| 2-19-1185 | S | N Ph | b2 | b5 | H |
| 2-19-1186 | S | N Ph | b3 | b5 | H |
| 2-19-1187 | S | N Ph | H | b6 | H |
| 2-19-1188 | S | N Ph | t-bu | b6 | H |
| 2-19-1189 | S | N Ph | methyl | b6 | H |
| 2-19-1190 | S | N Ph | b1 | b6 | H |
| 2-19-1191 | S | N Ph | b2 | b6 | H |
| 2-19-1192 | S | N Ph | b3 | b6 | H |
| 2-19-1193 | S | N Ph | H | b7 | H |
| 2-19-1194 | S | N Ph | t-bu | b7 | H |
| 2-19-1195 | S | N Ph | methyl | b7 | H |
| 2-19-1196 | S | N Ph | b1 | b7 | H |
| 2-19-1197 | S | N Ph | b2 | b7 | H |
| 2-19-1198 | S | N Ph | b3 | b7 | H |
| 2-19-1199 | S | N Ph | H | b8 | H |
| 2-19-1200 | S | N Ph | t-bu | b8 | H |
| 2-19-1201 | S | N Ph | methyl | b8 | H |
| 2-19-1202 | S | N Ph | b1 | b8 | H |
| 2-19-1203 | S | N Ph | b2 | b8 | H |
| 2-19-1204 | S | N Ph | b3 | b8 | H |
| 2-19-1205 | S | N Ph | H | b9 | H |
| 2-19-1206 | S | N Ph | t-bu | b9 | H |
| 2-19-1207 | S | N Ph | methyl | b9 | H |
| 2-19-1208 | S | N Ph | b1 | b9 | H |
| 2-19-1209 | S | N Ph | b2 | b9 | H |
| 2-19-1210 | S | N Ph | b3 | b9 | H |
| 2-19-1211 | S | N Ph | H | b10 | H |
| 2-19-1212 | S | N Ph | t-bu | b10 | H |
| 2-19-1213 | S | N Ph | methyl | b10 | H |
| 2-19-1214 | S | N Ph | b1 | b10 | H |
| 2-19-1215 | S | N Ph | b2 | b10 | H |
| 2-19-1216 | S | N Ph | b3 | b10 | H |
| 2-19-1217 | S | N Ph | H | a1 | a1 |
| 2-19-1218 | S | N Ph | t-bu | a1 | a1 |
| 2-19-1219 | S | N Ph | methyl | a1 | a1 |
| 2-19-1220 | S | N Ph | b1 | a1 | a1 |
| 2-19-1221 | S | N Ph | b2 | a1 | a1 |
| 2-19-1222 | S | N Ph | b3 | a1 | a1 |
| 2-19-1223 | S | N Ph | H | a2 | a2 |
| 2-19-1224 | S | N Ph | t-bu | a2 | a2 |
| 2-19-1225 | S | N Ph | methyl | a2 | a2 |
| 2-19-1226 | S | N Ph | b1 | a2 | a2 |
| 2-19-1227 | S | N Ph | b2 | a2 | a2 |
| 2-19-1228 | S | N Ph | b3 | a2 | a2 |
| 2-19-1229 | S | N Ph | H | a3 | a3 |
| 2-19-1230 | S | N Ph | t-bu | a3 | a3 |
| 2-19-1231 | S | N Ph | methyl | a3 | a3 |
| 2-19-1232 | S | N Ph | b1 | a3 | a3 |
| 2-19-1233 | S | N Ph | b2 | a3 | a3 |
| 2-19-1234 | S | N Ph | b3 | a3 | a3 |
| 2-19-1235 | S | N Ph | H | b1 | b1 |
| 2-19-1236 | S | N Ph | t-bu | b1 | b1 |
| 2-19-1237 | S | N Ph | methyl | b1 | b1 |
| 2-19-1238 | S | N Ph | b1 | b1 | b1 |
| 2-19-1239 | S | N Ph | b2 | b1 | b1 |
| 2-19-1240 | S | N Ph | b3 | b1 | b1 |
| 2-19-1241 | S | N Ph | H | b2 | b2 |
| 2-19-1242 | S | N Ph | t-bu | b2 | b2 |
| 2-19-1243 | S | N Ph | methyl | b2 | b2 |
| 2-19-1244 | S | N Ph | b1 | b2 | b2 |
| 2-19-1245 | S | N Ph | b2 | b2 | b2 |
| 2-19-1246 | S | N Ph | b3 | b2 | b2 |
| 2-19-1247 | S | N Ph | H | b3 | b3 |
| 2-19-1248 | S | N Ph | t-bu | b3 | b3 |
| 2-19-1249 | S | N Ph | methyl | b3 | b3 |
| 2-19-1250 | S | N Ph | b1 | b3 | b3 |
| 2-19-1251 | S | N Ph | b2 | b3 | b3 |
| 2-19-1252 | S | N Ph | b3 | b3 | b3 |
| 2-19-1253 | S | N Ph | H | b5 | b5 |
| 2-19-1254 | S | N Ph | t-bu | b5 | b5 |
| 2-19-1255 | S | N Ph | methyl | b5 | b5 |
| 2-19-1256 | S | N Ph | b1 | b5 | b5 |
| 2-19-1257 | S | N Ph | b2 | b5 | b5 |
| 2-19-1258 | S | N Ph | b3 | b5 | b5 |
| 2-19-1259 | S | N Ph | H | b6 | b6 |
| 2-19-1260 | S | N Ph | t-bu | b6 | b6 |
| 2-19-1261 | S | N Ph | methyl | b6 | b6 |
| 2-19-1262 | S | N Ph | b1 | b6 | b6 |
| 2-19-1263 | S | N Ph | b2 | b6 | b6 |
| 2-19-1264 | S | N Ph | b3 | b6 | b6 |
| 2-19-1265 | S | N Ph | H | b7 | b7 |
| 2-19-1266 | S | N Ph | t-bu | b7 | b7 |
| 2-19-1267 | S | N Ph | methyl | b7 | b7 |
| 2-19-1268 | S | N Ph | b1 | b7 | b7 |
| 2-19-1269 | S | N Ph | b2 | b7 | b7 |
| 2-19-1270 | S | N Ph | b3 | b7 | b7 |

### Group 2-20

The compounds are represented by the formula 2-20 below, wherein CY₁ and CY₂ are each a benzene group, X₁ is B, and Y1, Y2, Y3, R3a, (R1)a1, (R2)a2 are each shown in the table 20 below,

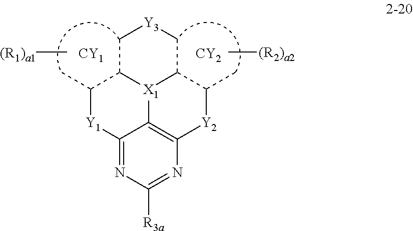

TABLE 20

| Compound | | | | | |
|---|---|---|---|---|---|
| No. | Y1/Y2 | Y3 | R3a | (R1)a1 | (R2)a2 |
| 2-20-001 | N-Ph | N-Ph | H | H | H |
| 2-20-002 | N-Ph | N-Ph | t-bu | H | H |
| 2-20-003 | N-Ph | N-Ph | methyl | H | H |
| 2-20-004 | N-Ph | N-Ph | t-bu | t-bu | t-bu |
| 2-20-005 | N-Ph | N-Ph | methyl | methyl | methyl |
| 2-20-006 | N-Ph | N-Ph | b1 | H | H |
| 2-20-007 | N-Ph | N-Ph | b2 | H | H |
| 2-20-008 | N-Ph | N-Ph | b3 | H | H |
| 2-20-009 | N-Ph | N-Ph | H | H | a1 |
| 2-20-010 | N-Ph | N-Ph | t-bu | H | a1 |
| 2-20-011 | N-Ph | N-Ph | methyl | H | a1 |
| 2-20-012 | N-Ph | N-Ph | b1 | H | a1 |
| 2-20-013 | N-Ph | N-Ph | b2 | H | a1 |
| 2-20-014 | N-Ph | N-Ph | b3 | H | a1 |
| 2-20-015 | N-Ph | N-Ph | H | H | a2 |
| 2-20-016 | N-Ph | N-Ph | t-bu | H | a2 |
| 2-20-017 | N-Ph | N-Ph | methyl | H | a2 |
| 2-20-018 | N-Ph | N-Ph | b1 | H | a2 |
| 2-20-019 | N-Ph | N-Ph | b2 | H | a2 |
| 2-20-020 | N-Ph | N-Ph | b3 | H | a2 |
| 2-20-021 | N-Ph | N-Ph | H | H | a3 |
| 2-20-022 | N-Ph | N-Ph | t-bu | H | a3 |
| 2-20-023 | N-Ph | N-Ph | methyl | H | a3 |
| 2-20-024 | N-Ph | N-Ph | b1 | H | a3 |
| 2-20-025 | N-Ph | N-Ph | b2 | H | a3 |
| 2-20-026 | N-Ph | N-Ph | b3 | H | a3 |
| 2-20-027 | N-Ph | N-Ph | H | H | a4 |
| 2-20-028 | N-Ph | N-Ph | t-bu | H | a4 |
| 2-20-029 | N-Ph | N-Ph | methyl | H | a4 |
| 2-20-030 | N-Ph | N-Ph | b1 | H | a4 |
| 2-20-031 | N-Ph | N-Ph | b2 | H | a4 |
| 2-20-032 | N-Ph | N-Ph | b3 | H | a4 |
| 2-20-033 | N-Ph | N-Ph | H | H | a5 |
| 2-20-034 | N-Ph | N-Ph | t-bu | H | a5 |
| 2-20-035 | N-Ph | N-Ph | methyl | H | a5 |
| 2-20-036 | N-Ph | N-Ph | b1 | H | a5 |
| 2-20-037 | N-Ph | N-Ph | b2 | H | a5 |
| 2-20-038 | N-Ph | N-Ph | b3 | H | a5 |
| 2-20-039 | N-Ph | N-Ph | H | H | a6 |
| 2-20-040 | N-Ph | N-Ph | t-bu | H | a6 |
| 2-20-041 | N-Ph | N-Ph | methyl | H | a6 |
| 2-20-042 | N-Ph | N-Ph | b1 | H | a6 |
| 2-20-043 | N-Ph | N-Ph | b2 | H | a6 |
| 2-20-044 | N-Ph | N-Ph | b3 | H | a6 |
| 2-20-045 | N-Ph | N-Ph | H | H | b1 |
| 2-20-046 | N-Ph | N-Ph | t-bu | H | b1 |
| 2-20-047 | N-Ph | N-Ph | methyl | H | b1 |
| 2-20-048 | N-Ph | N-Ph | b1 | H | b1 |
| 2-20-049 | N-Ph | N-Ph | b2 | H | b1 |
| 2-20-050 | N-Ph | N-Ph | b3 | H | b1 |
| 2-20-051 | N-Ph | N-Ph | H | H | b2 |
| 2-20-052 | N-Ph | N-Ph | t-bu | H | b2 |
| 2-20-053 | N-Ph | N-Ph | methyl | H | b2 |
| 2-20-054 | N-Ph | N-Ph | b1 | H | b2 |
| 2-20-055 | N-Ph | N-Ph | b2 | H | b2 |
| 2-20-056 | N-Ph | N-Ph | b3 | H | b2 |
| 2-20-057 | N-Ph | N-Ph | H | H | b3 |
| 2-20-058 | N-Ph | N-Ph | t-bu | H | b3 |
| 2-20-059 | N-Ph | N-Ph | methyl | H | b3 |
| 2-20-060 | N-Ph | N-Ph | b1 | H | b3 |
| 2-20-061 | N-Ph | N-Ph | b2 | H | b3 |
| 2-20-062 | N-Ph | N-Ph | b3 | H | b3 |
| 2-20-063 | N-Ph | N-Ph | H | H | b4 |
| 2-20-064 | N-Ph | N-Ph | t-bu | H | b4 |
| 2-20-065 | N-Ph | N-Ph | methyl | H | b4 |
| 2-20-066 | N-Ph | N-Ph | b1 | H | b4 |
| 2-20-067 | N-Ph | N-Ph | b2 | H | b4 |
| 2-20-068 | N-Ph | N-Ph | b3 | H | b4 |
| 2-20-069 | N-Ph | N-Ph | H | H | b5 |
| 2-20-070 | N-Ph | N-Ph | t-bu | H | b5 |
| 2-20-071 | N-Ph | N-Ph | methyl | H | b5 |
| 2-20-072 | N-Ph | N-Ph | b1 | H | b5 |
| 2-20-073 | N-Ph | N-Ph | b2 | H | b5 |
| 2-20-074 | N-Ph | N-Ph | b3 | H | b5 |
| 2-20-075 | N-Ph | N-Ph | H | H | b6 |
| 2-20-076 | N-Ph | N-Ph | t-bu | H | b6 |
| 2-20-077 | N-Ph | N-Ph | methyl | H | b6 |
| 2-20-078 | N-Ph | N-Ph | b1 | H | b6 |
| 2-20-079 | N-Ph | N-Ph | b2 | H | b6 |
| 2-20-080 | N-Ph | N-Ph | b3 | H | b6 |
| 2-20-081 | N-Ph | N-Ph | H | H | b7 |
| 2-20-082 | N-Ph | N-Ph | t-bu | H | b7 |
| 2-20-083 | N-Ph | N-Ph | methyl | H | b7 |
| 2-20-084 | N-Ph | N-Ph | b1 | H | b7 |
| 2-20-085 | N-Ph | N-Ph | b2 | H | b7 |
| 2-20-086 | N-Ph | N-Ph | b3 | H | b7 |
| 2-20-087 | N-Ph | N-Ph | H | H | b8 |
| 2-20-088 | N-Ph | N-Ph | t-bu | H | b8 |
| 2-20-089 | N-Ph | N-Ph | methyl | H | b8 |
| 2-20-090 | N-Ph | N-Ph | b1 | H | b8 |
| 2-20-091 | N-Ph | N-Ph | b2 | H | b8 |
| 2-20-092 | N-Ph | N-Ph | b3 | H | b8 |
| 2-20-093 | N-Ph | N-Ph | H | H | b9 |
| 2-20-094 | N-Ph | N-Ph | t-bu | H | b9 |
| 2-20-095 | N-Ph | N-Ph | methyl | H | b9 |
| 2-20-096 | N-Ph | N-Ph | b1 | H | b9 |
| 2-20-097 | N-Ph | N-Ph | b2 | H | b9 |
| 2-20-098 | N-Ph | N-Ph | b3 | H | b9 |
| 2-20-099 | N-Ph | N-Ph | H | H | b10 |
| 2-20-100 | N-Ph | N-Ph | t-bu | H | b10 |
| 2-20-101 | N-Ph | N-Ph | methyl | H | b10 |
| 2-20-102 | N-Ph | N-Ph | b1 | H | b10 |
| 2-20-103 | N-Ph | N-Ph | b2 | H | b10 |
| 2-20-104 | N-Ph | N-Ph | b3 | H | b10 |
| 2-20-105 | N-Ph | N-Ph | H | a1 | H |
| 2-20-106 | N-Ph | N-Ph | t-bu | a1 | H |
| 2-20-107 | N-Ph | N-Ph | methyl | a1 | H |
| 2-20-108 | N-Ph | N-Ph | b1 | a1 | H |
| 2-20-109 | N-Ph | N-Ph | b2 | a1 | H |
| 2-20-110 | N-Ph | N-Ph | b3 | a1 | H |
| 2-20-111 | N-Ph | N-Ph | H | a2 | H |
| 2-20-112 | N-Ph | N-Ph | t-bu | a2 | H |
| 2-20-113 | N-Ph | N-Ph | methyl | a2 | H |
| 2-20-114 | N-Ph | N-Ph | b1 | a2 | H |
| 2-20-115 | N-Ph | N-Ph | b2 | a2 | H |
| 2-20-116 | N-Ph | N-Ph | b3 | a2 | H |
| 2-20-117 | N-Ph | N-Ph | H | a3 | H |
| 2-20-118 | N-Ph | N-Ph | t-bu | a3 | H |
| 2-20-119 | N-Ph | N-Ph | methyl | a3 | H |
| 2-20-120 | N-Ph | N-Ph | b1 | a3 | H |
| 2-20-121 | N-Ph | N-Ph | b2 | a3 | H |
| 2-20-122 | N-Ph | N-Ph | b3 | a3 | H |
| 2-20-123 | N-Ph | N-Ph | H | a4 | H |
| 2-20-124 | N-Ph | N-Ph | t-bu | a4 | H |
| 2-20-125 | N-Ph | N-Ph | methyl | a4 | H |
| 2-20-126 | N-Ph | N-Ph | b1 | a4 | H |
| 2-20-127 | N-Ph | N-Ph | b2 | a4 | H |
| 2-20-128 | N-Ph | N-Ph | b3 | a4 | H |
| 2-20-129 | N-Ph | N-Ph | H | a5 | H |
| 2-20-130 | N-Ph | N-Ph | t-bu | a5 | H |
| 2-20-131 | N-Ph | N-Ph | methyl | a5 | H |
| 2-20-132 | N-Ph | N-Ph | b1 | a5 | H |
| 2-20-133 | N-Ph | N-Ph | b2 | a5 | H |
| 2-20-134 | N-Ph | N-Ph | b3 | a5 | H |
| 2-20-135 | N-Ph | N-Ph | H | a6 | H |
| 2-20-136 | N-Ph | N-Ph | t-bu | a6 | H |
| 2-20-137 | N-Ph | N-Ph | methyl | a6 | H |
| 2-20-138 | N-Ph | N-Ph | b1 | a6 | H |
| 2-20-139 | N-Ph | N-Ph | b2 | a6 | H |
| 2-20-140 | N-Ph | N-Ph | b3 | a6 | H |
| 2-20-141 | N-Ph | N-Ph | H | b1 | H |
| 2-20-142 | N-Ph | N-Ph | t-bu | b1 | H |
| 2-20-143 | N-Ph | N-Ph | methyl | b1 | H |
| 2-20-144 | N-Ph | N-Ph | b1 | b1 | H |
| 2-20-145 | N-Ph | N-Ph | b2 | b1 | H |
| 2-20-146 | N-Ph | N-Ph | b3 | b1 | H |
| 2-20-147 | N-Ph | N-Ph | H | b2 | H |
| 2-20-148 | N-Ph | N-Ph | t-bu | b2 | H |
| 2-20-149 | N-Ph | N-Ph | methyl | b2 | H |
| 2-20-150 | N-Ph | N-Ph | b1 | b2 | H |
| 2-20-151 | N-Ph | N-Ph | b2 | b2 | H |
| 2-20-152 | N-Ph | N-Ph | b3 | b2 | H |
| 2-20-153 | N-Ph | N-Ph | H | b3 | H |
| 2-20-154 | N-Ph | N-Ph | t-bu | b3 | H |
| 2-20-155 | N-Ph | N-Ph | methyl | b3 | H |
| 2-20-156 | N-Ph | N-Ph | b1 | b3 | H |
| 2-20-157 | N-Ph | N-Ph | b2 | b3 | H |
| 2-20-158 | N-Ph | N-Ph | b3 | b3 | H |
| 2-20-159 | N-Ph | N-Ph | H | b4 | H |
| 2-20-160 | N-Ph | N-Ph | t-bu | b4 | H |
| 2-20-161 | N-Ph | N-Ph | methyl | b4 | H |
| 2-20-162 | N-Ph | N-Ph | b1 | b4 | H |
| 2-20-163 | N-Ph | N-Ph | b2 | b4 | H |
| 2-20-164 | N-Ph | N-Ph | b3 | b4 | H |
| 2-20-165 | N-Ph | N-Ph | H | b5 | H |
| 2-20-166 | N-Ph | N-Ph | t-bu | b5 | H |
| 2-20-167 | N-Ph | N-Ph | methyl | b5 | H |
| 2-20-168 | N-Ph | N-Ph | b1 | b5 | H |
| 2-20-169 | N-Ph | N-Ph | b2 | b5 | H |
| 2-20-170 | N-Ph | N-Ph | b3 | b5 | H |
| 2-20-171 | N-Ph | N-Ph | H | b6 | H |
| 2-20-172 | N-Ph | N-Ph | t-bu | b6 | H |
| 2-20-173 | N-Ph | N-Ph | methyl | b6 | H |
| 2-20-174 | N-Ph | N-Ph | b1 | b6 | H |
| 2-20-175 | N-Ph | N-Ph | b2 | b6 | H |
| 2-20-176 | N-Ph | N-Ph | b3 | b6 | H |
| 2-20-177 | N-Ph | N-Ph | H | b7 | H |
| 2-20-178 | N-Ph | N-Ph | t-bu | b7 | H |
| 2-20-179 | N-Ph | N-Ph | methyl | b7 | H |
| 2-20-180 | N-Ph | N-Ph | b1 | b7 | H |
| 2-20-181 | N-Ph | N-Ph | b2 | b7 | H |
| 2-20-182 | N-Ph | N-Ph | b3 | b7 | H |
| 2-20-183 | N-Ph | N-Ph | H | b8 | H |
| 2-20-184 | N-Ph | N-Ph | t-bu | b8 | H |
| 2-20-185 | N-Ph | N-Ph | methyl | b8 | H |
| 2-20-186 | N-Ph | N-Ph | b1 | b8 | H |
| 2-20-187 | N-Ph | N-Ph | b2 | b8 | H |
| 2-20-188 | N-Ph | N-Ph | b3 | b8 | H |
| 2-20-189 | N-Ph | N-Ph | H | b9 | H |
| 2-20-190 | N-Ph | N-Ph | t-bu | b9 | H |
| 2-20-191 | N-Ph | N-Ph | methyl | b9 | H |
| 2-20-192 | N-Ph | N-Ph | b1 | b9 | H |
| 2-20-193 | N-Ph | N-Ph | b2 | b9 | H |
| 2-20-194 | N-Ph | N-Ph | b3 | b9 | H |
| 2-20-195 | N-Ph | N-Ph | H | b10 | H |
| 2-20-196 | N-Ph | N-Ph | t-bu | b10 | H |
| 2-20-197 | N-Ph | N-Ph | methyl | b10 | H |
| 2-20-198 | N-Ph | N-Ph | b1 | b10 | H |
| 2-20-199 | N-Ph | N-Ph | b2 | b10 | H |
| 2-20-200 | N-Ph | N-Ph | b3 | b10 | H |
| 2-20-201 | N-Ph | N-Ph | H | a1 | a1 |
| 2-20-202 | N-Ph | N-Ph | t-bu | a1 | a1 |
| 2-20-203 | N-Ph | N-Ph | methyl | a1 | a1 |
| 2-20-204 | N-Ph | N-Ph | b1 | a1 | a1 |
| 2-20-205 | N-Ph | N-Ph | b2 | a1 | a1 |
| 2-20-206 | N-Ph | N-Ph | b3 | a1 | a1 |
| 2-20-207 | N-Ph | N-Ph | H | a2 | a2 |
| 2-20-208 | N-Ph | N-Ph | t-bu | a2 | a2 |
| 2-20-209 | N-Ph | N-Ph | methyl | a2 | a2 |
| 2-20-210 | N-Ph | N-Ph | b1 | a2 | a2 |
| 2-20-211 | N-Ph | N-Ph | b2 | a2 | a2 |
| 2-20-212 | N-Ph | N-Ph | b3 | a2 | a2 |
| 2-20-213 | N-Ph | N-Ph | H | a3 | a3 |
| 2-20-214 | N-Ph | N-Ph | t-bu | a3 | a3 |
| 2-20-215 | N-Ph | N-Ph | methyl | a3 | a3 |
| 2-20-216 | N-Ph | N-Ph | b1 | a3 | a3 |
| 2-20-217 | N-Ph | N-Ph | b2 | a3 | a3 |
| 2-20-218 | N-Ph | N-Ph | b3 | a3 | a3 |
| 2-20-219 | N-Ph | N-Ph | H | b1 | b1 |
| 2-20-220 | N-Ph | N-Ph | t-bu | b1 | b1 |
| 2-20-221 | N-Ph | N-Ph | methyl | b1 | b1 |
| 2-20-222 | N-Ph | N-Ph | b1 | b1 | b1 |
| 2-20-223 | N-Ph | N-Ph | b2 | b1 | b1 |
| 2-20-224 | N-Ph | N-Ph | b3 | b1 | b1 |
| 2-20-225 | N-Ph | N-Ph | H | b2 | b2 |
| 2-20-226 | N-Ph | N-Ph | t-bu | b2 | b2 |
| 2-20-227 | N-Ph | N-Ph | methyl | b2 | b2 |
| 2-20-228 | N-Ph | N-Ph | b1 | b2 | b2 |
| 2-20-229 | N-Ph | N-Ph | b2 | b2 | b2 |
| 2-20-230 | N-Ph | N-Ph | b3 | b2 | b2 |
| 2-20-231 | N-Ph | N-Ph | H | b3 | b3 |
| 2-20-232 | N-Ph | N-Ph | t-bu | b3 | b3 |
| 2-20-233 | N-Ph | N-Ph | methyl | b3 | b3 |
| 2-20-234 | N-Ph | N-Ph | b1 | b3 | b3 |
| 2-20-235 | N-Ph | N-Ph | b2 | b3 | b3 |
| 2-20-236 | N-Ph | N-Ph | b3 | b3 | b3 |
| 2-20-237 | N-Ph | N-Ph | H | b5 | b5 |
| 2-20-238 | N-Ph | N-Ph | t-bu | b5 | b5 |
| 2-20-239 | N-Ph | N-Ph | methyl | b5 | b5 |
| 2-20-240 | N-Ph | N-Ph | b1 | b5 | b5 |
| 2-20-241 | N-Ph | N-Ph | b2 | b5 | b5 |
| 2-20-242 | N-Ph | N-Ph | b3 | b5 | b5 |
| 2-20-243 | N-Ph | N-Ph | H | b6 | b6 |
| 2-20-244 | N-Ph | N-Ph | t-bu | b6 | b6 |
| 2-20-245 | N-Ph | N-Ph | methyl | b6 | b6 |
| 2-20-246 | N-Ph | N-Ph | b1 | b6 | b6 |
| 2-20-247 | N-Ph | N-Ph | b2 | b6 | b6 |
| 2-20-248 | N-Ph | N-Ph | b3 | b6 | b6 |
| 2-20-249 | N-Ph | N-Ph | H | b7 | b7 |
| 2-20-250 | N-Ph | N-Ph | t-bu | b7 | b7 |
| 2-20-251 | N-Ph | N-Ph | methyl | b7 | b7 |
| 2-20-252 | N-Ph | N-Ph | b1 | b7 | b7 |
| 2-20-253 | N-Ph | N-Ph | b2 | b7 | b7 |
| 2-20-254 | N-Ph | N-Ph | b3 | b7 | b7 |
| 2-20-255 | N-tolyl | N-tolyl | H | H | H |
| 2-20-256 | N-tolyl | N-tolyl | t-bu | H | H |
| 2-20-257 | N-tolyl | N-tolyl | methyl | H | H |
| 2-20-258 | N-tolyl | N-tolyl | t-bu | t-bu | t-bu |
| 2-20-259 | N-tolyl | N-tolyl | methyl | methyl | methyl |
| 2-20-260 | N-tolyl | N-tolyl | b1 | H | H |
| 2-20-261 | N-tolyl | N-tolyl | b2 | H | H |
| 2-20-262 | N-tolyl | N-tolyl | b3 | H | H |
| 2-20-263 | N-tolyl | N-tolyl | H | H | a1 |
| 2-20-264 | N-tolyl | N-tolyl | t-bu | H | a1 |
| 2-20-265 | N-tolyl | N-tolyl | methyl | H | a1 |
| 2-20-266 | N-tolyl | N-tolyl | b1 | H | a1 |
| 2-20-267 | N-tolyl | N-tolyl | b2 | H | a1 |
| 2-20-268 | N-tolyl | N-tolyl | b3 | H | a1 |
| 2-20-269 | N-tolyl | N-tolyl | H | H | a2 |
| 2-20-270 | N-tolyl | N-tolyl | t-bu | H | a2 |
| 2-20-271 | N-tolyl | N-tolyl | methyl | H | a2 |
| 2-20-272 | N-tolyl | N-tolyl | b1 | H | a2 |
| 2-20-273 | N-tolyl | N-tolyl | b2 | H | a2 |
| 2-20-274 | N-tolyl | N-tolyl | b3 | H | a2 |
| 2-20-275 | N-tolyl | N-tolyl | H | H | a3 |
| 2-20-276 | N-tolyl | N-tolyl | t-bu | H | a3 |
| 2-20-277 | N-tolyl | N-tolyl | methyl | H | a3 |
| 2-20-278 | N-tolyl | N-tolyl | b1 | H | a3 |
| 2-20-279 | N-tolyl | N-tolyl | b2 | H | a3 |
| 2-20-280 | N-tolyl | N-tolyl | b3 | H | a3 |
| 2-20-281 | N-tolyl | N-tolyl | H | H | a4 |
| 2-20-282 | N-tolyl | N-tolyl | t-bu | H | a4 |
| 2-20-283 | N-tolyl | N-tolyl | methyl | H | a4 |
| 2-20-284 | N-tolyl | N-tolyl | b1 | H | a4 |
| 2-20-285 | N-tolyl | N-tolyl | b2 | H | a4 |
| 2-20-286 | N-tolyl | N-tolyl | b3 | H | a4 |
| 2-20-287 | N-tolyl | N-tolyl | H | H | a5 |
| 2-20-288 | N-tolyl | N-tolyl | t-bu | H | a5 |
| 2-20-289 | N-tolyl | N-tolyl | methyl | H | a5 |
| 2-20-290 | N-tolyl | N-tolyl | b1 | H | a5 |
| 2-20-291 | N-tolyl | N-tolyl | b2 | H | a5 |
| 2-20-292 | N-tolyl | N-tolyl | b3 | H | a5 |
| 2-20-293 | N-tolyl | N-tolyl | H | H | a6 |
| 2-20-294 | N-tolyl | N-tolyl | t-bu | H | a6 |
| 2-20-295 | N-tolyl | N-tolyl | methyl | H | a6 |
| 2-20-296 | N-tolyl | N-tolyl | b1 | H | a6 |
| 2-20-297 | N-tolyl | N-tolyl | b2 | H | a6 |
| 2-20-298 | N-tolyl | N-tolyl | b3 | H | a6 |
| 2-20-299 | N-tolyl | N-tolyl | H | H | b1 |
| 2-20-300 | N-tolyl | N-tolyl | t-bu | H | b1 |
| 2-20-301 | N-tolyl | N-tolyl | methyl | H | b1 |
| 2-20-302 | N-tolyl | N-tolyl | b1 | H | b1 |
| 2-20-303 | N-tolyl | N-tolyl | b2 | H | b1 |
| 2-20-304 | N-tolyl | N-tolyl | b3 | H | b1 |
| 2-20-305 | N-tolyl | N-tolyl | H | H | b2 |
| 2-20-306 | N-tolyl | N-tolyl | t-bu | H | b2 |
| 2-20-307 | N-tolyl | N-tolyl | methyl | H | b2 |
| 2-20-308 | N-tolyl | N-tolyl | b1 | H | b2 |
| 2-20-309 | N-tolyl | N-tolyl | b2 | H | b2 |
| 2-20-310 | N-tolyl | N-tolyl | b3 | H | b2 |
| 2-20-311 | N-tolyl | N-tolyl | H | H | b3 |
| 2-20-312 | N-tolyl | N-tolyl | t-bu | H | b3 |
| 2-20-313 | N-tolyl | N-tolyl | methyl | H | b3 |
| 2-20-314 | N-tolyl | N-tolyl | b1 | H | b3 |
| 2-20-315 | N-tolyl | N-tolyl | b2 | H | b3 |
| 2-20-316 | N-tolyl | N-tolyl | b3 | H | b3 |
| 2-20-317 | N-tolyl | N-tolyl | H | H | b4 |
| 2-20-318 | N-tolyl | N-tolyl | t-bu | H | b4 |
| 2-20-319 | N-tolyl | N-tolyl | methyl | H | b4 |
| 2-20-320 | N-tolyl | N-tolyl | b1 | H | b4 |
| 2-20-321 | N-tolyl | N-tolyl | b2 | H | b4 |
| 2-20-322 | N-tolyl | N-tolyl | b3 | H | b4 |
| 2-20-323 | N-tolyl | N-tolyl | H | H | b5 |
| 2-20-324 | N-tolyl | N-tolyl | t-bu | H | b5 |
| 2-20-325 | N-tolyl | N-tolyl | methyl | H | b5 |
| 2-20-326 | N-tolyl | N-tolyl | b1 | H | b5 |
| 2-20-327 | N-tolyl | N-tolyl | b2 | H | b5 |
| 2-20-328 | N-tolyl | N-tolyl | b3 | H | b5 |
| 2-20-329 | N-tolyl | N-tolyl | H | H | b6 |
| 2-20-330 | N-tolyl | N-tolyl | t-bu | H | b6 |
| 2-20-331 | N-tolyl | N-tolyl | methyl | H | b6 |
| 2-20-332 | N-tolyl | N-tolyl | b1 | H | b6 |
| 2-20-333 | N-tolyl | N-tolyl | b2 | H | b6 |
| 2-20-334 | N-tolyl | N-tolyl | b3 | H | b6 |
| 2-20-335 | N-tolyl | N-tolyl | H | H | b7 |
| 2-20-336 | N-tolyl | N-tolyl | t-bu | H | b7 |
| 2-20-337 | N-tolyl | N-tolyl | methyl | H | b7 |
| 2-20-338 | N-tolyl | N-tolyl | b1 | H | b7 |
| 2-20-339 | N-tolyl | N-tolyl | b2 | H | b7 |
| 2-20-340 | N-tolyl | N-tolyl | b3 | H | b7 |
| 2-20-341 | N-tolyl | N-tolyl | H | H | b8 |
| 2-20-342 | N-tolyl | N-tolyl | t-bu | H | b8 |
| 2-20-343 | N-tolyl | N-tolyl | methyl | H | b8 |
| 2-20-344 | N-tolyl | N-tolyl | b1 | H | b8 |
| 2-20-345 | N-tolyl | N-tolyl | b2 | H | b8 |
| 2-20-346 | N-tolyl | N-tolyl | b3 | H | b8 |
| 2-20-347 | N-tolyl | N-tolyl | H | H | b9 |
| 2-20-348 | N-tolyl | N-tolyl | t-bu | H | b9 |
| 2-20-349 | N-tolyl | N-tolyl | methyl | H | b9 |
| 2-20-350 | N-tolyl | N-tolyl | b1 | H | b9 |
| 2-20-351 | N-tolyl | N-tolyl | b2 | H | b9 |
| 2-20-352 | N-tolyl | N-tolyl | b3 | H | b9 |
| 2-20-353 | N-tolyl | N-tolyl | H | H | b10 |
| 2-20-354 | N-tolyl | N-tolyl | t-bu | H | b10 |
| 2-20-355 | N-tolyl | N-tolyl | methyl | H | b10 |
| 2-20-356 | N-tolyl | N-tolyl | b1 | H | b10 |
| 2-20-357 | N-tolyl | N-tolyl | b2 | H | b10 |
| 2-20-358 | N-tolyl | N-tolyl | b3 | H | b10 |
| 2-20-359 | N-tolyl | N-tolyl | H | a1 | H |
| 2-20-360 | N-tolyl | N-tolyl | t-bu | a1 | H |
| 2-20-361 | N-tolyl | N-tolyl | methyl | a1 | H |
| 2-20-362 | N-tolyl | N-tolyl | b1 | a1 | H |
| 2-20-363 | N-tolyl | N-tolyl | b2 | a1 | H |
| 2-20-364 | N-tolyl | N-tolyl | b3 | a1 | H |
| 2-20-365 | N-tolyl | N-tolyl | H | a2 | H |
| 2-20-366 | N-tolyl | N-tolyl | t-bu | a2 | H |
| 2-20-367 | N-tolyl | N-tolyl | methyl | a2 | H |
| 2-20-368 | N-tolyl | N-tolyl | b1 | a2 | H |
| 2-20-369 | N-tolyl | N-tolyl | b2 | a2 | H |
| 2-20-370 | N-tolyl | N-tolyl | b3 | a2 | H |
| 2-20-371 | N-tolyl | N-tolyl | H | a3 | H |
| 2-20-372 | N-tolyl | N-tolyl | t-bu | a3 | H |
| 2-20-373 | N-tolyl | N-tolyl | methyl | a3 | H |
| 2-20-374 | N-tolyl | N-tolyl | b1 | a3 | H |
| 2-20-375 | N-tolyl | N-tolyl | b2 | a3 | H |
| 2-20-376 | N-tolyl | N-tolyl | b3 | a3 | H |
| 2-20-377 | N-tolyl | N-tolyl | H | a4 | H |
| 2-20-378 | N-tolyl | N-tolyl | t-bu | a4 | H |
| 2-20-379 | N-tolyl | N-tolyl | methyl | a4 | H |
| 2-20-380 | N-tolyl | N-tolyl | b1 | a4 | H |
| 2-20-381 | N-tolyl | N-tolyl | b2 | a4 | H |
| 2-20-382 | N-tolyl | N-tolyl | b3 | a4 | H |
| 2-20-383 | N-tolyl | N-tolyl | H | a5 | H |
| 2-20-384 | N-tolyl | N-tolyl | t-bu | a5 | H |
| 2-20-385 | N-tolyl | N-tolyl | methyl | a5 | H |
| 2-20-386 | N-tolyl | N-tolyl | b1 | a5 | H |
| 2-20-387 | N-tolyl | N-tolyl | b2 | a5 | H |
| 2-20-388 | N-tolyl | N-tolyl | b3 | a5 | H |
| 2-20-389 | N-tolyl | N-tolyl | H | a6 | H |
| 2-20-390 | N-tolyl | N-tolyl | t-bu | a6 | H |
| 2-20-391 | N-tolyl | N-tolyl | methyl | a6 | H |
| 2-20-392 | N-tolyl | N-tolyl | b1 | a6 | H |
| 2-20-393 | N-tolyl | N-tolyl | b2 | a6 | H |
| 2-20-394 | N-tolyl | N-tolyl | b3 | a6 | H |
| 2-20-395 | N-tolyl | N-tolyl | H | b1 | H |
| 2-20-396 | N-tolyl | N-tolyl | t-bu | b1 | H |
| 2-20-397 | N-tolyl | N-tolyl | methyl | b1 | H |
| 2-20-398 | N-tolyl | N-tolyl | b1 | b1 | H |
| 2-20-399 | N-tolyl | N-tolyl | b2 | b1 | H |
| 2-20-400 | N-tolyl | N-tolyl | b3 | b1 | H |
| 2-20-401 | N-tolyl | N-tolyl | H | b2 | H |
| 2-20-402 | N-tolyl | N-tolyl | t-bu | b2 | H |
| 2-20-403 | N-tolyl | N-tolyl | methyl | b2 | H |
| 2-20-404 | N-tolyl | N-tolyl | b1 | b2 | H |
| 2-20-405 | N-tolyl | N-tolyl | b2 | b2 | H |
| 2-20-406 | N-tolyl | N-tolyl | b3 | b2 | H |
| 2-20-407 | N-tolyl | N-tolyl | H | b3 | H |
| 2-20-408 | N-tolyl | N-tolyl | t-bu | b3 | H |
| 2-20-409 | N-tolyl | N-tolyl | methyl | b3 | H |
| 2-20-410 | N-tolyl | N-tolyl | b1 | b3 | H |
| 2-20-411 | N-tolyl | N-tolyl | b2 | b3 | H |
| 2-20-412 | N-tolyl | N-tolyl | b3 | b3 | H |
| 2-20-413 | N-tolyl | N-tolyl | H | b4 | H |
| 2-20-414 | N-tolyl | N-tolyl | t-bu | b4 | H |
| 2-20-415 | N-tolyl | N-tolyl | methyl | b4 | H |
| 2-20-416 | N-tolyl | N-tolyl | b1 | b4 | H |
| 2-20-417 | N-tolyl | N-tolyl | b2 | b4 | H |
| 2-20-418 | N-tolyl | N-tolyl | b3 | b4 | H |
| 2-20-419 | N-tolyl | N-tolyl | H | b5 | H |
| 2-20-420 | N-tolyl | N-tolyl | t-bu | b5 | H |
| 2-20-421 | N-tolyl | N-tolyl | methyl | b5 | H |
| 2-20-422 | N-tolyl | N-tolyl | b1 | b5 | H |
| 2-20-423 | N-tolyl | N-tolyl | b2 | b5 | H |
| 2-20-424 | N-tolyl | N-tolyl | b3 | b5 | H |
| 2-20-425 | N-tolyl | N-tolyl | H | b6 | H |
| 2-20-426 | N-tolyl | N-tolyl | t-bu | b6 | H |
| 2-20-427 | N-tolyl | N-tolyl | methyl | b6 | H |
| 2-20-428 | N-tolyl | N-tolyl | b1 | b6 | H |
| 2-20-429 | N-tolyl | N-tolyl | b2 | b6 | H |
| 2-20-430 | N-tolyl | N-tolyl | b3 | b6 | H |
| 2-20-431 | N-tolyl | N-tolyl | H | b7 | H |
| 2-20-432 | N-tolyl | N-tolyl | t-bu | b7 | H |
| 2-20-433 | N-tolyl | N-tolyl | methyl | b7 | H |
| 2-20-434 | N-tolyl | N-tolyl | b1 | b7 | H |
| 2-20-435 | N-tolyl | N-tolyl | b2 | b7 | H |
| 2-20-436 | N-tolyl | N-tolyl | b3 | b7 | H |
| 2-20-437 | N-tolyl | N-tolyl | H | b8 | H |
| 2-20-438 | N-tolyl | N-tolyl | t-bu | b8 | H |
| 2-20-439 | N-tolyl | N-tolyl | methyl | b8 | H |
| 2-20-440 | N-tolyl | N-tolyl | b1 | b8 | H |
| 2-20-441 | N-tolyl | N-tolyl | b2 | b8 | H |
| 2-20-442 | N-tolyl | N-tolyl | b3 | b8 | H |
| 2-20-443 | N-tolyl | N-tolyl | H | b9 | H |
| 2-20-444 | N-tolyl | N-tolyl | t-bu | b9 | H |
| 2-20-445 | N-tolyl | N-tolyl | methyl | b9 | H |
| 2-20-446 | N-tolyl | N-tolyl | b1 | b9 | H |
| 2-20-447 | N-tolyl | N-tolyl | b2 | b9 | H |
| 2-20-448 | N-tolyl | N-tolyl | b3 | b9 | H |
| 2-20-449 | N-tolyl | N-tolyl | H | b10 | H |
| 2-20-450 | N-tolyl | N-tolyl | t-bu | b10 | H |
| 2-20-451 | N-tolyl | N-tolyl | methyl | b10 | H |
| 2-20-452 | N-tolyl | N-tolyl | b1 | b10 | H |
| 2-20-453 | N-tolyl | N-tolyl | b2 | b10 | H |
| 2-20-454 | N-tolyl | N-tolyl | b3 | b10 | H |
| 2-20-455 | N-tolyl | N-tolyl | H | a1 | a1 |
| 2-20-456 | N-tolyl | N-tolyl | t-bu | a1 | a1 |
| 2-20-457 | N-tolyl | N-tolyl | methyl | a1 | a1 |
| 2-20-458 | N-tolyl | N-tolyl | b1 | a1 | a1 |
| 2-20-459 | N-tolyl | N-tolyl | b2 | a1 | a1 |
| 2-20-460 | N-tolyl | N-tolyl | b3 | a1 | a1 |
| 2-20-461 | N-tolyl | N-tolyl | H | a2 | a2 |
| 2-20-462 | N-tolyl | N-tolyl | t-bu | a2 | a2 |
| 2-20-463 | N-tolyl | N-tolyl | methyl | a2 | a2 |
| 2-20-464 | N-tolyl | N-tolyl | b1 | a2 | a2 |
| 2-20-465 | N-tolyl | N-tolyl | b2 | a2 | a2 |
| 2-20-466 | N-tolyl | N-tolyl | b3 | a2 | a2 |
| 2-20-467 | N-tolyl | N-tolyl | H | a3 | a3 |
| 2-20-468 | N-tolyl | N-tolyl | t-bu | a3 | a3 |
| 2-20-469 | N-tolyl | N-tolyl | methyl | a3 | a3 |
| 2-20-470 | N-tolyl | N-tolyl | b1 | a3 | a3 |
| 2-20-471 | N-tolyl | N-tolyl | b2 | a3 | a3 |
| 2-20-472 | N-tolyl | N-tolyl | b3 | a3 | a3 |
| 2-20-473 | N-tolyl | N-tolyl | H | b1 | b1 |
| 2-20-474 | N-tolyl | N-tolyl | t-bu | b1 | b1 |
| 2-20-475 | N-tolyl | N-tolyl | methyl | b1 | b1 |
| 2-20-476 | N-tolyl | N-tolyl | b1 | b1 | b1 |
| 2-20-477 | N-tolyl | N-tolyl | b2 | b1 | b1 |
| 2-20-478 | N-tolyl | N-tolyl | b3 | b1 | b1 |
| 2-20-479 | N-tolyl | N-tolyl | H | b2 | b2 |
| 2-20-480 | N-tolyl | N-tolyl | t-bu | b2 | b2 |
| 2-20-481 | N-tolyl | N-tolyl | methyl | b2 | b2 |
| 2-20-482 | N-tolyl | N-tolyl | b1 | b2 | b2 |
| 2-20-483 | N-tolyl | N-tolyl | b2 | b2 | b2 |
| 2-20-484 | N-tolyl | N-tolyl | b3 | b2 | b2 |
| 2-20-485 | N-tolyl | N-tolyl | H | b3 | b3 |
| 2-20-486 | N-tolyl | N-tolyl | t-bu | b3 | b3 |
| 2-20-487 | N-tolyl | N-tolyl | methyl | b3 | b3 |
| 2-20-488 | N-tolyl | N-tolyl | b1 | b3 | b3 |
| 2-20-489 | N-tolyl | N-tolyl | b2 | b3 | b3 |
| 2-20-490 | N-tolyl | N-tolyl | b3 | b3 | b3 |
| 2-20-491 | N-tolyl | N-tolyl | H | b5 | b5 |
| 2-20-492 | N-tolyl | N-tolyl | t-bu | b5 | b5 |
| 2-20-493 | N-tolyl | N-tolyl | methyl | b5 | b5 |
| 2-20-494 | N-tolyl | N-tolyl | b1 | b5 | b5 |
| 2-20-495 | N-tolyl | N-tolyl | b2 | b5 | b5 |
| 2-20-496 | N-tolyl | N-tolyl | b3 | b5 | b5 |
| 2-20-497 | N-tolyl | N-tolyl | H | b6 | b6 |
| 2-20-498 | N-tolyl | N-tolyl | t-bu | b6 | b6 |
| 2-20-499 | N-tolyl | N-tolyl | methyl | b6 | b6 |
| 2-20-500 | N-tolyl | N-tolyl | b1 | b6 | b6 |
| 2-20-501 | N-tolyl | N-tolyl | b2 | b6 | b6 |
| 2-20-502 | N-tolyl | N-tolyl | b3 | b6 | b6 |
| 2-20-503 | N-tolyl | N-tolyl | H | b7 | b7 |
| 2-20-504 | N-tolyl | N-tolyl | t-bu | b7 | b7 |
| 2-20-505 | N-tolyl | N-tolyl | methyl | b7 | b7 |
| 2-20-506 | N-tolyl | N-tolyl | b1 | b7 | b7 |
| 2-20-507 | N-tolyl | N-tolyl | b2 | b7 | b7 |
| 2-20-508 | N-tolyl | N-tolyl | b3 | b7 | b7 |
| 2-20-509 | N-Biph | N-Biph | H | H | H |
| 2-20-510 | N-Biph | N-Biph | t-bu | H | H |
| 2-20-511 | N-Biph | N-Biph | methyl | H | H |
| 2-20-512 | N-Biph | N-Biph | t-bu | t-bu | t-bu |
| 2-20-513 | N-Biph | N-Biph | methyl | methyl | methyl |
| 2-20-514 | N-Biph | N-Biph | b1 | H | H |
| 2-20-515 | N-Biph | N-Biph | b2 | H | H |
| 2-20-516 | N-Biph | N-Biph | b3 | H | H |
| 2-20-517 | N-Biph | N-Biph | H | H | a1 |
| 2-20-518 | N-Biph | N-Biph | t-bu | H | a1 |
| 2-20-519 | N-Biph | N-Biph | methyl | H | a1 |
| 2-20-520 | N-Biph | N-Biph | b1 | H | a1 |
| 2-20-521 | N-Biph | N-Biph | b2 | H | a1 |
| 2-20-522 | N-Biph | N-Biph | b3 | H | a1 |
| 2-20-523 | N-Biph | N-Biph | H | H | a2 |
| 2-20-524 | N-Biph | N-Biph | t-bu | H | a2 |
| 2-20-525 | N-Biph | N-Biph | methyl | H | a2 |
| 2-20-526 | N-Biph | N-Biph | b1 | H | a2 |
| 2-20-527 | N-Biph | N-Biph | b2 | H | a2 |
| 2-20-528 | N-Biph | N-Biph | b3 | H | a2 |
| 2-20-529 | N-Biph | N-Biph | H | H | a3 |
| 2-20-530 | N-Biph | N-Biph | t-bu | H | a3 |
| 2-20-531 | N-Biph | N-Biph | methyl | H | a3 |
| 2-20-532 | N-Biph | N-Biph | b1 | H | a3 |
| 2-20-533 | N-Biph | N-Biph | b2 | H | a3 |
| 2-20-534 | N-Biph | N-Biph | b3 | H | a3 |
| 2-20-535 | N-Biph | N-Biph | H | H | a4 |
| 2-20-536 | N-Biph | N-Biph | t-bu | H | a4 |
| 2-20-537 | N-Biph | N-Biph | methyl | H | a4 |
| 2-20-538 | N-Biph | N-Biph | b1 | H | a4 |
| 2-20-539 | N-Biph | N-Biph | b2 | H | a4 |
| 2-20-540 | N-Biph | N-Biph | b3 | H | a4 |
| 2-20-541 | N-Biph | N-Biph | H | H | a5 |
| 2-20-542 | N-Biph | N-Biph | t-bu | H | a5 |
| 2-20-543 | N-Biph | N-Biph | methyl | H | a5 |
| 2-20-544 | N-Biph | N-Biph | b1 | H | a5 |
| 2-20-545 | N-Biph | N-Biph | b2 | H | a5 |
| 2-20-546 | N-Biph | N-Biph | b3 | H | a5 |
| 2-20-547 | N-Biph | N-Biph | H | H | a6 |
| 2-20-548 | N-Biph | N-Biph | t-bu | H | a6 |
| 2-20-549 | N-Biph | N-Biph | methyl | H | a6 |
| 2-20-550 | N-Biph | N-Biph | b1 | H | a6 |
| 2-20-551 | N-Biph | N-Biph | b2 | H | a6 |
| 2-20-552 | N-Biph | N-Biph | b3 | H | a6 |
| 2-20-553 | N-Biph | N-Biph | H | H | b1 |
| 2-20-554 | N-Biph | N-Biph | t-bu | H | b1 |
| 2-20-555 | N-Biph | N-Biph | methyl | H | b1 |
| 2-20-556 | N-Biph | N-Biph | b1 | H | b1 |
| 2-20-557 | N-Biph | N-Biph | b2 | H | b1 |
| 2-20-558 | N-Biph | N-Biph | b3 | H | b1 |
| 2-20-559 | N-Biph | N-Biph | H | H | b2 |
| 2-20-560 | N-Biph | N-Biph | t-bu | H | b2 |
| 2-20-561 | N-Biph | N-Biph | methyl | H | b2 |
| 2-20-562 | N-Biph | N-Biph | b1 | H | b2 |
| 2-20-563 | N-Biph | N-Biph | b2 | H | b2 |
| 2-20-564 | N-Biph | N-Biph | b3 | H | b2 |
| 2-20-565 | N-Biph | N-Biph | H | H | b3 |
| 2-20-566 | N-Biph | N-Biph | t-bu | H | b3 |
| 2-20-567 | N-Biph | N-Biph | methyl | H | b3 |
| 2-20-568 | N-Biph | N-Biph | b1 | H | b3 |
| 2-20-569 | N-Biph | N-Biph | b2 | H | b3 |
| 2-20-570 | N-Biph | N-Biph | b3 | H | b3 |
| 2-20-571 | N-Biph | N-Biph | H | H | b4 |
| 2-20-572 | N-Biph | N-Biph | t-bu | H | b4 |
| 2-20-573 | N-Biph | N-Biph | methyl | H | b4 |
| 2-20-574 | N-Biph | N-Biph | b1 | H | b4 |
| 2-20-575 | N-Biph | N-Biph | b2 | H | b4 |
| 2-20-576 | N-Biph | N-Biph | b3 | H | b4 |
| 2-20-577 | N-Biph | N-Biph | H | H | b5 |
| 2-20-578 | N-Biph | N-Biph | t-bu | H | b5 |
| 2-20-579 | N-Biph | N-Biph | methyl | H | b5 |
| 2-20-580 | N-Biph | N-Biph | b1 | H | b5 |
| 2-20-581 | N-Biph | N-Biph | b2 | H | b5 |
| 2-20-582 | N-Biph | N-Biph | b3 | H | b5 |
| 2-20-583 | N-Biph | N-Biph | H | H | b6 |
| 2-20-584 | N-Biph | N-Biph | t-bu | H | b6 |
| 2-20-585 | N-Biph | N-Biph | methyl | H | b6 |
| 2-20-586 | N-Biph | N-Biph | b1 | H | b6 |
| 2-20-587 | N-Biph | N-Biph | b2 | H | b6 |
| 2-20-588 | N-Biph | N-Biph | b3 | H | b6 |
| 2-20-589 | N-Biph | N-Biph | H | H | b7 |
| 2-20-590 | N-Biph | N-Biph | t-bu | H | b7 |
| 2-20-591 | N-Biph | N-Biph | methyl | H | b7 |
| 2-20-592 | N-Biph | N-Biph | b1 | H | b7 |
| 2-20-593 | N-Biph | N-Biph | b2 | H | b7 |
| 2-20-594 | N-Biph | N-Biph | b3 | H | b7 |
| 2-20-595 | N-Biph | N-Biph | H | H | b8 |
| 2-20-596 | N-Biph | N-Biph | t-bu | H | b8 |
| 2-20-597 | N-Biph | N-Biph | methyl | H | b8 |
| 2-20-598 | N-Biph | N-Biph | b1 | H | b8 |
| 2-20-599 | N-Biph | N-Biph | b2 | H | b8 |
| 2-20-600 | N-Biph | N-Biph | b3 | H | b8 |
| 2-20-601 | N-Biph | N-Biph | H | H | b9 |
| 2-20-602 | N-Biph | N-Biph | t-bu | H | b9 |
| 2-20-603 | N-Biph | N-Biph | methyl | H | b9 |
| 2-20-604 | N-Biph | N-Biph | b1 | H | b9 |
| 2-20-605 | N-Biph | N-Biph | b2 | H | b9 |
| 2-20-606 | N-Biph | N-Biph | b3 | H | b9 |
| 2-20-607 | N-Biph | N-Biph | H | H | b10 |
| 2-20-608 | N-Biph | N-Biph | t-bu | H | b10 |
| 2-20-609 | N-Biph | N-Biph | methyl | H | b10 |
| 2-20-610 | N-Biph | N-Biph | b1 | H | b10 |
| 2-20-611 | N-Biph | N-Biph | b2 | H | b10 |
| 2-20-612 | N-Biph | N-Biph | b3 | H | b10 |
| 2-20-613 | N-Biph | N-Biph | H | a1 | H |
| 2-20-614 | N-Biph | N-Biph | t-bu | a1 | H |
| 2-20-615 | N-Biph | N-Biph | methyl | a1 | H |
| 2-20-616 | N-Biph | N-Biph | b1 | a1 | H |
| 2-20-617 | N-Biph | N-Biph | b2 | a1 | H |
| 2-20-618 | N-Biph | N-Biph | b3 | a1 | H |
| 2-20-619 | N-Biph | N-Biph | H | a2 | H |
| 2-20-620 | N-Biph | N-Biph | t-bu | a2 | H |
| 2-20-621 | N-Biph | N-Biph | methyl | a2 | H |
| 2-20-622 | N-Biph | N-Biph | b1 | a2 | H |
| 2-20-623 | N-Biph | N-Biph | b2 | a2 | H |
| 2-20-624 | N-Biph | N-Biph | b3 | a2 | H |
| 2-20-625 | N-Biph | N-Biph | H | a3 | H |
| 2-20-626 | N-Biph | N-Biph | t-bu | a3 | H |
| 2-20-627 | N-Biph | N-Biph | methyl | a3 | H |
| 2-20-628 | N-Biph | N-Biph | b1 | a3 | H |
| 2-20-629 | N-Biph | N-Biph | b2 | a3 | H |
| 2-20-630 | N-Biph | N-Biph | b3 | a3 | H |
| 2-20-631 | N-Biph | N-Biph | H | a4 | H |
| 2-20-632 | N-Biph | N-Biph | t-bu | a4 | H |
| 2-20-633 | N-Biph | N-Biph | methyl | a4 | H |
| 2-20-634 | N-Biph | N-Biph | b1 | a4 | H |
| 2-20-635 | N-Biph | N-Biph | b2 | a4 | H |
| 2-20-636 | N-Biph | N-Biph | b3 | a4 | H |
| 2-20-637 | N-Biph | N-Biph | H | a5 | H |
| 2-20-638 | N-Biph | N-Biph | t-bu | a5 | H |
| 2-20-639 | N-Biph | N-Biph | methyl | a5 | H |
| 2-20-640 | N-Biph | N-Biph | b1 | a5 | H |
| 2-20-641 | N-Biph | N-Biph | b2 | a5 | H |
| 2-20-642 | N-Biph | N-Biph | b3 | a5 | H |
| 2-20-643 | N-Biph | N-Biph | H | a6 | H |
| 2-20-644 | N-Biph | N-Biph | t-bu | a6 | H |
| 2-20-645 | N-Biph | N-Biph | methyl | a6 | H |
| 2-20-646 | N-Biph | N-Biph | b1 | a6 | H |
| 2-20-647 | N-Biph | N-Biph | b2 | a6 | H |
| 2-20-648 | N-Biph | N-Biph | b3 | a6 | H |
| 2-20-649 | N-Biph | N-Biph | H | b1 | H |
| 2-20-650 | N-Biph | N-Biph | t-bu | b1 | H |
| 2-20-651 | N-Biph | N-Biph | methyl | b1 | H |
| 2-20-652 | N-Biph | N-Biph | b1 | b1 | H |
| 2-20-653 | N-Biph | N-Biph | b2 | b1 | H |
| 2-20-654 | N-Biph | N-Biph | b3 | b1 | H |
| 2-20-655 | N-Biph | N-Biph | H | b2 | H |
| 2-20-656 | N-Biph | N-Biph | t-bu | b2 | H |
| 2-20-657 | N-Biph | N-Biph | methyl | b2 | H |
| 2-20-658 | N-Biph | N-Biph | b1 | b2 | H |
| 2-20-659 | N-Biph | N-Biph | b2 | b2 | H |
| 2-20-660 | N-Biph | N-Biph | b3 | b2 | H |
| 2-20-661 | N-Biph | N-Biph | H | b3 | H |
| 2-20-662 | N-Biph | N-Biph | t-bu | b3 | H |
| 2-20-663 | N-Biph | N-Biph | methyl | b3 | H |
| 2-20-664 | N-Biph | N-Biph | b1 | b3 | H |
| 2-20-665 | N-Biph | N-Biph | b2 | b3 | H |
| 2-20-666 | N-Biph | N-Biph | b3 | b3 | H |
| 2-20-667 | N-Biph | N-Biph | H | b4 | H |
| 2-20-668 | N-Biph | N-Biph | t-bu | b4 | H |
| 2-20-669 | N-Biph | N-Biph | methyl | b4 | H |
| 2-20-670 | N-Biph | N-Biph | b1 | b4 | H |
| 2-20-671 | N-Biph | N-Biph | b2 | b4 | H |
| 2-20-672 | N-Biph | N-Biph | b3 | b4 | H |
| 2-20-673 | N-Biph | N-Biph | H | b5 | H |
| 2-20-674 | N-Biph | N-Biph | t-bu | b5 | H |
| 2-20-675 | N-Biph | N-Biph | methyl | b5 | H |
| 2-20-676 | N-Biph | N-Biph | b1 | b5 | H |
| 2-20-677 | N-Biph | N-Biph | b2 | b5 | H |
| 2-20-678 | N-Biph | N-Biph | b3 | b5 | H |
| 2-20-679 | N-Biph | N-Biph | H | b6 | H |
| 2-20-680 | N-Biph | N-Biph | t-bu | b6 | H |
| 2-20-681 | N-Biph | N-Biph | methyl | b6 | H |
| 2-20-682 | N-Biph | N-Biph | b1 | b6 | H |
| 2-20-683 | N-Biph | N-Biph | b2 | b6 | H |
| 2-20-684 | N-Biph | N-Biph | b3 | b6 | H |
| 2-20-685 | N-Biph | N-Biph | H | b7 | H |
| 2-20-686 | N-Biph | N-Biph | t-bu | b7 | H |
| 2-20-687 | N-Biph | N-Biph | methyl | b7 | H |
| 2-20-688 | N-Biph | N-Biph | b1 | b7 | H |
| 2-20-689 | N-Biph | N-Biph | b2 | b7 | H |
| 2-20-690 | N-Biph | N-Biph | b3 | b7 | H |
| 2-20-691 | N-Biph | N-Biph | H | b8 | H |
| 2-20-692 | N-Biph | N-Biph | t-bu | b8 | H |
| 2-20-693 | N-Biph | N-Biph | methyl | b8 | H |
| 2-20-694 | N-Biph | N-Biph | b1 | b8 | H |
| 2-20-695 | N-Biph | N-Biph | b2 | b8 | H |
| 2-20-696 | N-Biph | N-Biph | b3 | b8 | H |
| 2-20-697 | N-Biph | N-Biph | H | b9 | H |
| 2-20-698 | N-Biph | N-Biph | t-bu | b9 | H |
| 2-20-699 | N-Biph | N-Biph | methyl | b9 | H |
| 2-20-700 | N-Biph | N-Biph | b1 | b9 | H |
| 2-20-701 | N-Biph | N-Biph | b2 | b9 | H |
| 2-20-702 | N-Biph | N-Biph | b3 | b9 | H |
| 2-20-703 | N-Biph | N-Biph | H | b10 | H |
| 2-20-704 | N-Biph | N-Biph | t-bu | b10 | H |
| 2-20-705 | N-Biph | N-Biph | methyl | b10 | H |
| 2-20-706 | N-Biph | N-Biph | b1 | b10 | H |
| 2-20-707 | N-Biph | N-Biph | b2 | b10 | H |
| 2-20-708 | N-Biph | N-Biph | b3 | b10 | H |
| 2-20-709 | N-Biph | N-Biph | H | a1 | a1 |
| 2-20-710 | N-Biph | N-Biph | t-bu | a1 | a1 |
| 2-20-711 | N-Biph | N-Biph | methyl | a1 | a1 |
| 2-20-712 | N-Biph | N-Biph | b1 | a1 | a1 |
| 2-20-713 | N-Biph | N-Biph | b2 | a1 | a1 |
| 2-20-714 | N-Biph | N-Biph | b3 | a1 | a1 |
| 2-20-715 | N-Biph | N-Biph | H | a2 | a2 |
| 2-20-716 | N-Biph | N-Biph | t-bu | a2 | a2 |
| 2-20-717 | N-Biph | N-Biph | methyl | a2 | a2 |
| 2-20-718 | N-Biph | N-Biph | b1 | a2 | a2 |
| 2-20-719 | N-Biph | N-Biph | b2 | a2 | a2 |
| 2-20-720 | N-Biph | N-Biph | b3 | a2 | a2 |
| 2-20-721 | N-Biph | N-Biph | H | a3 | a3 |
| 2-20-722 | N-Biph | N-Biph | t-bu | a3 | a3 |
| 2-20-723 | N-Biph | N-Biph | methyl | a3 | a3 |
| 2-20-724 | N-Biph | N-Biph | b1 | a3 | a3 |
| 2-20-725 | N-Biph | N-Biph | b2 | a3 | a3 |
| 2-20-726 | N-Biph | N-Biph | b3 | a3 | a3 |
| 2-20-727 | N-Biph | N-Biph | H | b1 | b1 |
| 2-20-728 | N-Biph | N-Biph | t-bu | b1 | b1 |
| 2-20-729 | N-Biph | N-Biph | methyl | b1 | b1 |
| 2-20-730 | N-Biph | N-Biph | b1 | b1 | b1 |
| 2-20-731 | N-Biph | N-Biph | b2 | b1 | b1 |
| 2-20-732 | N-Biph | N-Biph | b3 | b1 | b1 |
| 2-20-733 | N-Biph | N-Biph | H | b2 | b2 |
| 2-20-734 | N-Biph | N-Biph | t-bu | b2 | b2 |
| 2-20-735 | N-Biph | N-Biph | methyl | b2 | b2 |
| 2-20-736 | N-Biph | N-Biph | b1 | b2 | b2 |
| 2-20-737 | N-Biph | N-Biph | b2 | b2 | b2 |
| 2-20-738 | N-Biph | N-Biph | b3 | b2 | b2 |
| 2-20-739 | N-Biph | N-Biph | H | b3 | b3 |
| 2-20-740 | N-Biph | N-Biph | t-bu | b3 | b3 |
| 2-20-741 | N-Biph | N-Biph | methyl | b3 | b3 |
| 2-20-742 | N-Biph | N-Biph | b1 | b3 | b3 |
| 2-20-743 | N-Biph | N-Biph | b2 | b3 | b3 |
| 2-20-744 | N-Biph | N-Biph | b3 | b3 | b3 |
| 2-20-745 | N-Biph | N-Biph | H | b5 | b5 |
| 2-20-746 | N-Biph | N-Biph | t-bu | b5 | b5 |
| 2-20-747 | N-Biph | N-Biph | methyl | b5 | b5 |
| 2-20-748 | N-Biph | N-Biph | b1 | b5 | b5 |
| 2-20-749 | N-Biph | N-Biph | b2 | b5 | b5 |
| 2-20-750 | N-Biph | N-Biph | b3 | b5 | b5 |
| 2-20-751 | N-Biph | N-Biph | H | b6 | b6 |
| 2-20-752 | N-Biph | N-Biph | t-bu | b6 | b6 |
| 2-20-753 | N-Biph | N-Biph | methyl | b6 | b6 |
| 2-20-754 | N-Biph | N-Biph | b1 | b6 | b6 |
| 2-20-755 | N-Biph | N-Biph | b2 | b6 | b6 |
| 2-20-756 | N-Biph | N-Biph | b3 | b6 | b6 |
| 2-20-757 | N-Biph | N-Biph | H | b7 | b7 |
| 2-20-758 | N-Biph | N-Biph | t-bu | b7 | b7 |
| 2-20-759 | N-Biph | N-Biph | methyl | b7 | b7 |
| 2-20-760 | N-Biph | N-Biph | b1 | b7 | b7 |
| 2-20-761 | N-Biph | N-Biph | b2 | b7 | b7 |
| 2-20-762 | N-Biph | N-Biph | b3 | b7 | b7 |
| 2-20-763 | N-Ph | O | H | H | H |
| 2-20-764 | N-Ph | O | t-bu | H | H |
| 2-20-765 | N-Ph | O | methyl | H | H |
| 2-20-766 | N-Ph | O | t-bu | t-bu | t-bu |
| 2-20-767 | N-Ph | O | methyl | methyl | methyl |
| 2-20-768 | N-Ph | O | b1 | H | H |
| 2-20-769 | N-Ph | O | b2 | H | H |
| 2-20-770 | N-Ph | O | b3 | H | H |
| 2-20-771 | N-Ph | O | H | H | a1 |
| 2-20-772 | N-Ph | O | t-bu | H | a1 |
| 2-20-773 | N-Ph | O | methyl | H | a1 |
| 2-20-774 | N-Ph | O | b1 | H | a1 |
| 2-20-775 | N-Ph | O | b2 | H | a1 |
| 2-20-776 | N-Ph | O | b3 | H | a1 |
| 2-20-777 | N-Ph | O | H | H | a2 |
| 2-20-778 | N-Ph | O | t-bu | H | a2 |
| 2-20-779 | N-Ph | O | methyl | H | a2 |
| 2-20-780 | N-Ph | O | b1 | H | a2 |
| 2-20-781 | N-Ph | O | b2 | H | a2 |
| 2-20-782 | N-Ph | O | b3 | H | a2 |
| 2-20-783 | N-Ph | O | H | H | a3 |
| 2-20-784 | N-Ph | O | t-bu | H | a3 |
| 2-20-785 | N-Ph | O | methyl | H | a3 |
| 2-20-786 | N-Ph | O | b1 | H | a3 |
| 2-20-787 | N-Ph | O | b2 | H | a3 |
| 2-20-788 | N-Ph | O | b3 | H | a3 |
| 2-20-789 | N-Ph | O | H | H | a4 |
| 2-20-790 | N-Ph | O | t-bu | H | a4 |
| 2-20-791 | N-Ph | O | methyl | H | a4 |
| 2-20-792 | N-Ph | O | b1 | H | a4 |
| 2-20-793 | N-Ph | O | b2 | H | a4 |
| 2-20-794 | N-Ph | O | b3 | H | a4 |
| 2-20-795 | N-Ph | O | H | H | a5 |
| 2-20-796 | N-Ph | O | t-bu | H | a5 |
| 2-20-797 | N-Ph | O | methyl | H | a5 |
| 2-20-798 | N-Ph | O | b1 | H | a5 |
| 2-20-799 | N-Ph | O | b2 | H | a5 |
| 2-20-800 | N-Ph | O | b3 | H | a5 |
| 2-20-801 | N-Ph | O | H | H | a6 |
| 2-20-802 | N-Ph | O | t-bu | H | a6 |
| 2-20-803 | N-Ph | O | methyl | H | a6 |
| 2-20-804 | N-Ph | O | b1 | H | a6 |
| 2-20-805 | N-Ph | O | b2 | H | a6 |
| 2-20-806 | N-Ph | O | b3 | H | a6 |
| 2-20-807 | N-Ph | O | H | H | b1 |
| 2-20-808 | N-Ph | O | t-bu | H | b1 |
| 2-20-809 | N-Ph | O | methyl | H | b1 |
| 2-20-810 | N-Ph | O | b1 | H | b1 |
| 2-20-811 | N-Ph | O | b2 | H | b1 |
| 2-20-812 | N-Ph | O | b3 | H | b1 |
| 2-20-813 | N-Ph | O | H | H | b2 |
| 2-20-814 | N-Ph | O | t-bu | H | b2 |
| 2-20-815 | N-Ph | O | methyl | H | b2 |
| 2-20-816 | N-Ph | O | b1 | H | b2 |
| 2-20-817 | N-Ph | O | b2 | H | b2 |
| 2-20-818 | N-Ph | O | b3 | H | b2 |
| 2-20-819 | N-Ph | O | H | H | b3 |
| 2-20-820 | N-Ph | O | t-bu | H | b3 |
| 2-20-821 | N-Ph | O | methyl | H | b3 |
| 2-20-822 | N-Ph | O | b1 | H | b3 |
| 2-20-823 | N-Ph | O | b2 | H | b3 |
| 2-20-824 | N-Ph | O | b3 | H | b3 |
| 2-20-825 | N-Ph | O | H | H | b4 |
| 2-20-826 | N-Ph | O | t-bu | H | b4 |
| 2-20-827 | N-Ph | O | methyl | H | b4 |
| 2-20-828 | N-Ph | O | b1 | H | b4 |
| 2-20-829 | N-Ph | O | b2 | H | b4 |
| 2-20-830 | N-Ph | O | b3 | H | b4 |
| 2-20-831 | N-Ph | O | H | H | b5 |
| 2-20-832 | N-Ph | O | t-bu | H | b5 |
| 2-20-833 | N-Ph | O | methyl | H | b5 |
| 2-20-834 | N-Ph | O | b1 | H | b5 |
| 2-20-835 | N-Ph | O | b2 | H | b5 |
| 2-20-836 | N-Ph | O | b3 | H | b5 |
| 2-20-837 | N-Ph | O | H | H | b6 |
| 2-20-838 | N-Ph | O | t-bu | H | b6 |
| 2-20-839 | N-Ph | O | methyl | H | b6 |
| 2-20-840 | N-Ph | O | b1 | H | b6 |
| 2-20-841 | N-Ph | O | b2 | H | b6 |
| 2-20-842 | N-Ph | O | b3 | H | b6 |
| 2-20-843 | N-Ph | O | H | H | b7 |
| 2-20-844 | N-Ph | O | t-bu | H | b7 |
| 2-20-845 | N-Ph | O | methyl | H | b7 |
| 2-20-846 | N-Ph | O | b1 | H | b7 |
| 2-20-847 | N-Ph | O | b2 | H | b7 |
| 2-20-848 | N-Ph | O | b3 | H | b7 |
| 2-20-849 | N-Ph | O | H | H | b8 |
| 2-20-850 | N-Ph | O | t-bu | H | b8 |
| 2-20-851 | N-Ph | O | methyl | H | b8 |
| 2-20-852 | N-Ph | O | b1 | H | b8 |
| 2-20-853 | N-Ph | O | b2 | H | b8 |
| 2-20-854 | N-Ph | O | b3 | H | b8 |
| 2-20-855 | N-Ph | O | H | H | b9 |
| 2-20-856 | N-Ph | O | t-bu | H | b9 |
| 2-20-857 | N-Ph | O | methyl | H | b9 |
| 2-20-858 | N-Ph | O | b1 | H | b9 |
| 2-20-859 | N-Ph | O | b2 | H | b9 |
| 2-20-860 | N-Ph | O | b3 | H | b9 |
| 2-20-861 | N-Ph | O | H | H | b10 |
| 2-20-862 | N-Ph | O | t-bu | H | b10 |
| 2-20-863 | N-Ph | O | methyl | H | b10 |
| 2-20-864 | N-Ph | O | b1 | H | b10 |
| 2-20-865 | N-Ph | O | b2 | H | b10 |
| 2-20-866 | N-Ph | O | b3 | H | b10 |
| 2-20-867 | N-Ph | O | H | a1 | H |
| 2-20-868 | N-Ph | O | t-bu | a1 | H |
| 2-20-869 | N-Ph | O | methyl | a1 | H |
| 2-20-870 | N-Ph | O | b1 | a1 | H |
| 2-20-871 | N-Ph | O | b2 | a1 | H |
| 2-20-872 | N-Ph | O | b3 | a1 | H |
| 2-20-873 | N-Ph | O | H | a2 | H |
| 2-20-874 | N-Ph | O | t-bu | a2 | H |
| 2-20-875 | N-Ph | O | methyl | a2 | H |
| 2-20-876 | N-Ph | O | b1 | a2 | H |
| 2-20-877 | N-Ph | O | b2 | a2 | H |
| 2-20-878 | N-Ph | O | b3 | a2 | H |
| 2-20-879 | N-Ph | O | H | a3 | H |
| 2-20-880 | N-Ph | O | t-bu | a3 | H |
| 2-20-881 | N-Ph | O | methyl | a3 | H |
| 2-20-882 | N-Ph | O | b1 | a3 | H |
| 2-20-883 | N-Ph | O | b2 | a3 | H |
| 2-20-884 | N-Ph | O | b3 | a3 | H |
| 2-20-885 | N-Ph | O | H | a4 | H |
| 2-20-886 | N-Ph | O | t-bu | a4 | H |
| 2-20-887 | N-Ph | O | methyl | a4 | H |
| 2-20-888 | N-Ph | O | b1 | a4 | H |
| 2-20-889 | N-Ph | O | b2 | a4 | H |
| 2-20-890 | N-Ph | O | b3 | a4 | H |
| 2-20-891 | N-Ph | O | H | a5 | H |
| 2-20-892 | N-Ph | O | t-bu | a5 | H |
| 2-20-893 | N-Ph | O | methyl | a5 | H |
| 2-20-894 | N-Ph | O | b1 | a5 | H |
| 2-20-895 | N-Ph | O | b2 | a5 | H |
| 2-20-896 | N-Ph | O | b3 | a5 | H |
| 2-20-897 | N-Ph | O | H | a6 | H |
| 2-20-898 | N-Ph | O | t-bu | a6 | H |
| 2-20-899 | N-Ph | O | methyl | a6 | H |
| 2-20-900 | N-Ph | O | b1 | a6 | H |
| 2-20-901 | N-Ph | O | b2 | a6 | H |
| 2-20-902 | N-Ph | O | b3 | a6 | H |
| 2-20-903 | N-Ph | O | H | b1 | H |
| 2-20-904 | N-Ph | O | t-bu | b1 | H |
| 2-20-905 | N-Ph | O | methyl | b1 | H |
| 2-20-906 | N-Ph | O | b1 | b1 | H |
| 2-20-907 | N-Ph | O | b2 | b1 | H |
| 2-20-908 | N-Ph | O | b3 | b1 | H |
| 2-20-909 | N-Ph | O | H | b2 | H |
| 2-20-910 | N-Ph | O | t-bu | b2 | H |
| 2-20-911 | N-Ph | O | methyl | b2 | H |
| 2-20-912 | N-Ph | O | b1 | b2 | H |
| 2-20-913 | N-Ph | O | b2 | b2 | H |
| 2-20-914 | N-Ph | O | b3 | b2 | H |
| 2-20-915 | N-Ph | O | H | b3 | H |
| 2-20-916 | N-Ph | O | t-bu | b3 | H |
| 2-20-917 | N-Ph | O | methyl | b3 | H |
| 2-20-918 | N-Ph | O | b1 | b3 | H |
| 2-20-919 | N-Ph | O | b2 | b3 | H |
| 2-20-920 | N-Ph | O | b3 | b3 | H |
| 2-20-921 | N-Ph | O | H | b4 | H |
| 2-20-922 | N-Ph | O | t-bu | b4 | H |
| 2-20-923 | N-Ph | O | methyl | b4 | H |
| 2-20-924 | N-Ph | O | b1 | b4 | H |
| 2-20-925 | N-Ph | O | b2 | b4 | H |
| 2-20-926 | N-Ph | O | b3 | b4 | H |
| 2-20-927 | N-Ph | O | H | b5 | H |
| 2-20-928 | N-Ph | O | t-bu | b5 | H |
| 2-20-929 | N-Ph | O | methyl | b5 | H |
| 2-20-930 | N-Ph | O | b1 | b5 | H |
| 2-20-931 | N-Ph | O | b2 | b5 | H |
| 2-20-932 | N-Ph | O | b3 | b5 | H |
| 2-20-933 | N-Ph | O | H | b6 | H |
| 2-20-934 | N-Ph | O | t-bu | b6 | H |
| 2-20-935 | N-Ph | O | methyl | b6 | H |
| 2-20-936 | N-Ph | O | b1 | b6 | H |
| 2-20-937 | N-Ph | O | b2 | b6 | H |
| 2-20-938 | N-Ph | O | b3 | b6 | H |
| 2-20-939 | N-Ph | O | H | b7 | H |
| 2-20-940 | N-Ph | O | t-bu | b7 | H |
| 2-20-941 | N-Ph | O | methyl | b7 | H |
| 2-20-942 | N-Ph | O | b1 | b7 | H |
| 2-20-943 | N-Ph | O | b2 | b7 | H |
| 2-20-944 | N-Ph | O | b3 | b7 | H |
| 2-20-945 | N-Ph | O | H | b8 | H |
| 2-20-946 | N-Ph | O | t-bu | b8 | H |
| 2-20-947 | N-Ph | O | methyl | b8 | H |
| 2-20-948 | N-Ph | O | b1 | b8 | H |
| 2-20-949 | N-Ph | O | b2 | b8 | H |
| 2-20-950 | N-Ph | O | b3 | b8 | H |
| 2-20-951 | N-Ph | O | H | b9 | H |
| 2-20-952 | N-Ph | O | t-bu | b9 | H |
| 2-20-953 | N-Ph | O | methyl | b9 | H |
| 2-20-954 | N-Ph | O | b1 | b9 | H |
| 2-20-955 | N-Ph | O | b2 | b9 | H |
| 2-20-956 | N-Ph | O | b3 | b9 | H |
| 2-20-957 | N-Ph | O | H | b10 | H |
| 2-20-958 | N-Ph | O | t-bu | b10 | H |
| 2-20-959 | N-Ph | O | methyl | b10 | H |
| 2-20-960 | N-Ph | O | b1 | b10 | H |
| 2-20-961 | N-Ph | O | b2 | b10 | H |
| 2-20-962 | N-Ph | O | b3 | b10 | H |
| 2-20-963 | N-Ph | O | H | a1 | a1 |
| 2-20-964 | N-Ph | O | t-bu | a1 | a1 |
| 2-20-965 | N-Ph | O | methyl | a1 | a1 |
| 2-20-966 | N-Ph | O | b1 | a1 | a1 |
| 2-20-967 | N-Ph | O | b2 | a1 | a1 |
| 2-20-968 | N-Ph | O | b3 | a1 | a1 |
| 2-20-969 | N-Ph | O | H | a2 | a2 |
| 2-20-970 | N-Ph | O | t-bu | a2 | a2 |
| 2-20-971 | N-Ph | O | methyl | a2 | a2 |
| 2-20-972 | N-Ph | O | b1 | a2 | a2 |
| 2-20-973 | N-Ph | O | b2 | a2 | a2 |
| 2-20-974 | N-Ph | O | b3 | a2 | a2 |
| 2-20-975 | N-Ph | O | H | a3 | a3 |
| 2-20-976 | N-Ph | O | t-bu | a3 | a3 |
| 2-20-977 | N-Ph | O | methyl | a3 | a3 |
| 2-20-978 | N-Ph | O | b1 | a3 | a3 |
| 2-20-979 | N-Ph | O | b2 | a3 | a3 |
| 2-20-980 | N-Ph | O | b3 | a3 | a3 |
| 2-20-981 | N-Ph | O | H | b1 | b1 |
| 2-20-982 | N-Ph | O | t-bu | b1 | b1 |
| 2-20-983 | N-Ph | O | methyl | b1 | b1 |
| 2-20-984 | N-Ph | O | b1 | b1 | b1 |
| 2-20-985 | N-Ph | O | b2 | b1 | b1 |
| 2-20-986 | N-Ph | O | b3 | b1 | b1 |
| 2-20-987 | N-Ph | O | H | b2 | b2 |
| 2-20-988 | N-Ph | O | t-bu | b2 | b2 |
| 2-20-989 | N-Ph | O | methyl | b2 | b2 |
| 2-20-990 | N-Ph | O | b1 | b2 | b2 |
| 2-20-991 | N-Ph | O | b2 | b2 | b2 |
| 2-20-992 | N-Ph | O | b3 | b2 | b2 |
| 2-20-993 | N-Ph | O | H | b3 | b3 |
| 2-20-994 | N-Ph | O | t-bu | b3 | b3 |
| 2-20-995 | N-Ph | O | methyl | b3 | b3 |
| 2-20-996 | N-Ph | O | b1 | b3 | b3 |
| 2-20-997 | N-Ph | O | b2 | b3 | b3 |
| 2-20-998 | N-Ph | O | b3 | b3 | b3 |
| 2-20-999 | N-Ph | O | H | b5 | b5 |
| 2-20-1000 | N-Ph | O | t-bu | b5 | b5 |
| 2-20-1001 | N-Ph | O | methyl | b5 | b5 |
| 2-20-1002 | N-Ph | O | b1 | b5 | b5 |
| 2-20-1003 | N-Ph | O | b2 | b5 | b5 |
| 2-20-1004 | N-Ph | O | b3 | b5 | b5 |
| 2-20-1005 | N-Ph | O | H | b6 | b6 |
| 2-20-1006 | N-Ph | O | t-bu | b6 | b6 |
| 2-20-1007 | N-Ph | O | methyl | b6 | b6 |
| 2-20-1008 | N-Ph | O | b1 | b6 | b6 |
| 2-20-1009 | N-Ph | O | b2 | b6 | b6 |
| 2-20-1010 | N-Ph | O | b3 | b6 | b6 |
| 2-20-1011 | N-Ph | O | H | b7 | b7 |
| 2-20-1012 | N-Ph | O | t-bu | b7 | b7 |
| 2-20-1013 | N-Ph | O | methyl | b7 | b7 |
| 2-20-1014 | N-Ph | O | b1 | b7 | b7 |
| 2-20-1015 | N-Ph | O | b2 | b7 | b7 |
| 2-20-1016 | N-Ph | O | b3 | b7 | b7 |
| 2-20-1017 | N-Ph | S | H | H | H |
| 2-20-1018 | N-Ph | S | t-bu | H | H |
| 2-20-1019 | N-Ph | S | methyl | H | H |
| 2-20-1020 | N-Ph | S | t-bu | t-bu | t-bu |
| 2-20-1021 | N-Ph | S | methyl | methyl | methyl |
| 2-20-1022 | N-Ph | S | b1 | H | H |
| 2-20-1023 | N-Ph | S | b2 | H | H |
| 2-20-1024 | N-Ph | S | b3 | H | H |
| 2-20-1025 | N-Ph | S | H | H | a1 |
| 2-20-1026 | N-Ph | S | t-bu | H | a1 |
| 2-20-1027 | N-Ph | S | methyl | H | a1 |
| 2-20-1028 | N-Ph | S | b1 | H | a1 |
| 2-20-1029 | N-Ph | S | b2 | H | a1 |
| 2-20-1030 | N-Ph | S | b3 | H | a1 |
| 2-20-1031 | N-Ph | S | H | H | a2 |
| 2-20-1032 | N-Ph | S | t-bu | H | a2 |
| 2-20-1033 | N-Ph | S | methyl | H | a2 |
| 2-20-1034 | N-Ph | S | b1 | H | a2 |
| 2-20-1035 | N-Ph | S | b2 | H | a2 |
| 2-20-1036 | N-Ph | S | b3 | H | a2 |
| 2-20-1037 | N-Ph | S | H | H | a3 |
| 2-20-1038 | N-Ph | S | t-bu | H | a3 |
| 2-20-1039 | N-Ph | S | methyl | H | a3 |
| 2-20-1040 | N-Ph | S | b1 | H | a3 |
| 2-20-1041 | N-Ph | S | b2 | H | a3 |
| 2-20-1042 | N-Ph | S | b3 | H | a3 |
| 2-20-1043 | N-Ph | S | H | H | a4 |
| 2-20-1044 | N-Ph | S | t-bu | H | a4 |
| 2-20-1045 | N-Ph | S | methyl | H | a4 |
| 2-20-1046 | N-Ph | S | b1 | H | a4 |
| 2-20-1047 | N-Ph | S | b2 | H | a4 |
| 2-20-1048 | N-Ph | S | b3 | H | a4 |
| 2-20-1049 | N-Ph | S | H | H | a5 |
| 2-20-1050 | N-Ph | S | t-bu | H | a5 |
| 2-20-1051 | N-Ph | S | methyl | H | a5 |
| 2-20-1052 | N-Ph | S | b1 | H | a5 |
| 2-20-1053 | N-Ph | S | b2 | H | a5 |
| 2-20-1054 | N-Ph | S | b3 | H | a5 |
| 2-20-1055 | N-Ph | S | H | H | a6 |
| 2-20-1056 | N-Ph | S | t-bu | H | a6 |
| 2-20-1057 | N-Ph | S | methyl | H | a6 |
| 2-20-1058 | N-Ph | S | b1 | H | a6 |
| 2-20-1059 | N-Ph | S | b2 | H | a6 |
| 2-20-1060 | N-Ph | S | b3 | H | a6 |
| 2-20-1061 | N-Ph | S | H | H | b1 |
| 2-20-1062 | N-Ph | S | t-bu | H | b1 |
| 2-20-1063 | N-Ph | S | methyl | H | b1 |
| 2-20-1064 | N-Ph | S | b1 | H | b1 |
| 2-20-1065 | N-Ph | S | b2 | H | b1 |
| 2-20-1066 | N-Ph | S | b3 | H | b1 |
| 2-20-1067 | N-Ph | S | H | H | b2 |
| 2-20-1068 | N-Ph | S | t-bu | H | b2 |
| 2-20-1069 | N-Ph | S | methyl | H | b2 |
| 2-20-1070 | N-Ph | S | b1 | H | b2 |
| 2-20-1071 | N-Ph | S | b2 | H | b2 |
| 2-20-1072 | N-Ph | S | b3 | H | b2 |
| 2-20-1073 | N-Ph | S | H | H | b3 |
| 2-20-1074 | N-Ph | S | t-bu | H | b3 |
| 2-20-1075 | N-Ph | S | methyl | H | b3 |
| 2-20-1076 | N-Ph | S | b1 | H | b3 |
| 2-20-1077 | N-Ph | S | b2 | H | b3 |
| 2-20-1078 | N-Ph | S | b3 | H | b3 |
| 2-20-1079 | N-Ph | S | H | H | b4 |
| 2-20-1080 | N-Ph | S | t-bu | H | b4 |
| 2-20-1081 | N-Ph | S | methyl | H | b4 |
| 2-20-1082 | N-Ph | S | b1 | H | b4 |
| 2-20-1083 | N-Ph | S | b2 | H | b4 |
| 2-20-1084 | N-Ph | S | b3 | H | b4 |
| 2-20-1085 | N-Ph | S | H | H | b5 |
| 2-20-1086 | N-Ph | S | t-bu | H | b5 |
| 2-20-1087 | N-Ph | S | methyl | H | b5 |
| 2-20-1088 | N-Ph | S | b1 | H | b5 |
| 2-20-1089 | N-Ph | S | b2 | H | b5 |
| 2-20-1090 | N-Ph | S | b3 | H | b5 |
| 2-20-1091 | N-Ph | S | H | H | b6 |
| 2-20-1092 | N-Ph | S | t-bu | H | b6 |
| 2-20-1093 | N-Ph | S | methyl | H | b6 |
| 2-20-1094 | N-Ph | S | b1 | H | b6 |
| 2-20-1095 | N-Ph | S | b2 | H | b6 |
| 2-20-1096 | N-Ph | S | b3 | H | b6 |
| 2-20-1097 | N-Ph | S | H | H | b7 |
| 2-20-1098 | N-Ph | S | t-bu | H | b7 |
| 2-20-1099 | N-Ph | S | methyl | H | b7 |
| 2-20-1100 | N-Ph | S | b1 | H | b7 |
| 2-20-1101 | N-Ph | S | b2 | H | b7 |
| 2-20-1102 | N-Ph | S | b3 | H | b7 |
| 2-20-1103 | N-Ph | S | H | H | b8 |
| 2-20-1104 | N-Ph | S | t-bu | H | b8 |
| 2-20-1105 | N-Ph | S | methyl | H | b8 |
| 2-20-1106 | N-Ph | S | b1 | H | b8 |
| 2-20-1107 | N-Ph | S | b2 | H | b8 |
| 2-20-1108 | N-Ph | S | b3 | H | b8 |
| 2-20-1109 | N-Ph | S | H | H | b9 |
| 2-20-1110 | N-Ph | S | t-bu | H | b9 |
| 2-20-1111 | N-Ph | S | methyl | H | b9 |
| 2-20-1112 | N-Ph | S | b1 | H | b9 |
| 2-20-1113 | N-Ph | S | b2 | H | b9 |
| 2-20-1114 | N-Ph | S | b3 | H | b9 |
| 2-20-1115 | N-Ph | S | H | H | b10 |
| 2-20-1116 | N-Ph | S | t-bu | H | b10 |
| 2-20-1117 | N-Ph | S | methyl | H | b10 |
| 2-20-1118 | N-Ph | S | b1 | H | b10 |
| 2-20-1119 | N-Ph | S | b2 | H | b10 |
| 2-20-1120 | N-Ph | S | b3 | H | b10 |
| 2-20-1121 | N-Ph | S | H | a1 | H |
| 2-20-1122 | N-Ph | S | t-bu | a1 | H |
| 2-20-1123 | N-Ph | S | methyl | a1 | H |
| 2-20-1124 | N-Ph | S | b1 | a1 | H |
| 2-20-1125 | N-Ph | S | b2 | a1 | H |
| 2-20-1126 | N-Ph | S | b3 | a1 | H |
| 2-20-1127 | N-Ph | S | H | a2 | H |
| 2-20-1128 | N-Ph | S | t-bu | a2 | H |
| 2-20-1129 | N-Ph | S | methyl | a2 | H |
| 2-20-1130 | N-Ph | S | b1 | a2 | H |
| 2-20-1131 | N-Ph | S | b2 | a2 | H |
| 2-20-1132 | N-Ph | S | b3 | a2 | H |
| 2-20-1133 | N-Ph | S | H | a3 | H |
| 2-20-1134 | N-Ph | S | t-bu | a3 | H |
| 2-20-1135 | N-Ph | S | methyl | a3 | H |
| 2-20-1136 | N-Ph | S | b1 | a3 | H |
| 2-20-1137 | N-Ph | S | b2 | a3 | H |
| 2-20-1138 | N-Ph | S | b3 | a3 | H |
| 2-20-1139 | N-Ph | S | H | a4 | H |
| 2-20-1140 | N-Ph | S | t-bu | a4 | H |
| 2-20-1141 | N-Ph | S | methyl | a4 | H |
| 2-20-1142 | N-Ph | S | b1 | a4 | H |
| 2-20-1143 | N-Ph | S | b2 | a4 | H |
| 2-20-1144 | N-Ph | S | b3 | a4 | H |
| 2-20-1145 | N-Ph | S | H | a5 | H |
| 2-20-1146 | N-Ph | S | t-bu | a5 | H |
| 2-20-1147 | N-Ph | S | methyl | a5 | H |
| 2-20-1148 | N-Ph | S | b1 | a5 | H |
| 2-20-1149 | N-Ph | S | b2 | a5 | H |
| 2-20-1150 | N-Ph | S | b3 | a5 | H |
| 2-20-1151 | N-Ph | S | H | a6 | H |
| 2-20-1152 | N-Ph | S | t-bu | a6 | H |
| 2-20-1153 | N-Ph | S | methyl | a6 | H |
| 2-20-1154 | N-Ph | S | b1 | a6 | H |
| 2-20-1155 | N-Ph | S | b2 | a6 | H |
| 2-20-1156 | N-Ph | S | b3 | a6 | H |
| 2-20-1157 | N-Ph | S | H | b1 | H |
| 2-20-1158 | N-Ph | S | t-bu | b1 | H |
| 2-20-1159 | N-Ph | S | methyl | b1 | H |
| 2-20-1160 | N-Ph | S | b1 | b1 | H |
| 2-20-1161 | N-Ph | S | b2 | b1 | H |
| 2-20-1162 | N-Ph | S | b3 | b1 | H |
| 2-20-1163 | N-Ph | S | H | b2 | H |
| 2-20-1164 | N-Ph | S | t-bu | b2 | H |
| 2-20-1165 | N-Ph | S | methyl | b2 | H |
| 2-20-1166 | N-Ph | S | b1 | b2 | H |
| 2-20-1167 | N-Ph | S | b2 | b2 | H |
| 2-20-1168 | N-Ph | S | b3 | b2 | H |
| 2-20-1169 | N-Ph | S | H | b3 | H |
| 2-20-1170 | N-Ph | S | t-bu | b3 | H |
| 2-20-1171 | N-Ph | S | methyl | b3 | H |
| 2-20-1172 | N-Ph | S | b1 | b3 | H |
| 2-20-1173 | N-Ph | S | b2 | b3 | H |
| 2-20-1174 | N-Ph | S | b3 | b3 | H |
| 2-20-1175 | N-Ph | S | H | b4 | H |
| 2-20-1176 | N-Ph | S | t-bu | b4 | H |
| 2-20-1177 | N-Ph | S | methyl | b4 | H |
| 2-20-1178 | N-Ph | S | b1 | b4 | H |
| 2-20-1179 | N-Ph | S | b2 | b4 | H |
| 2-20-1180 | N-Ph | S | b3 | b4 | H |
| 2-20-1181 | N-Ph | S | H | b5 | H |
| 2-20-1182 | N-Ph | S | t-bu | b5 | H |
| 2-20-1183 | N-Ph | S | methyl | b5 | H |
| 2-20-1184 | N-Ph | S | b1 | b5 | H |
| 2-20-1185 | N-Ph | S | b2 | b5 | H |
| 2-20-1186 | N-Ph | S | b3 | b5 | H |
| 2-20-1187 | N-Ph | S | H | b6 | H |
| 2-20-1188 | N-Ph | S | t-bu | b6 | H |
| 2-20-1189 | N-Ph | S | methyl | b6 | H |
| 2-20-1190 | N-Ph | S | b1 | b6 | H |
| 2-20-1191 | N-Ph | S | b2 | b6 | H |
| 2-20-1192 | N-Ph | S | b3 | b6 | H |
| 2-20-1193 | N-Ph | S | H | b7 | H |
| 2-20-1194 | N-Ph | S | t-bu | b7 | H |
| 2-20-1195 | N-Ph | S | methyl | b7 | H |
| 2-20-1196 | N-Ph | S | b1 | b7 | H |
| 2-20-1197 | N-Ph | S | b2 | b7 | H |
| 2-20-1198 | N-Ph | S | b3 | b7 | H |
| 2-20-1199 | N-Ph | S | H | b8 | H |
| 2-20-1200 | N-Ph | S | t-bu | b8 | H |
| 2-20-1201 | N-Ph | S | methyl | b8 | H |
| 2-20-1202 | N-Ph | S | b1 | b8 | H |
| 2-20-1203 | N-Ph | S | b2 | b8 | H |
| 2-20-1204 | N-Ph | S | b3 | b8 | H |
| 2-20-1205 | N-Ph | S | H | b9 | H |
| 2-20-1206 | N-Ph | S | t-bu | b9 | H |
| 2-20-1207 | N-Ph | S | methyl | b9 | H |
| 2-20-1208 | N-Ph | S | b1 | b9 | H |
| 2-20-1209 | N-Ph | S | b2 | b9 | H |
| 2-20-1210 | N-Ph | S | b3 | b9 | H |
| 2-20-1211 | N-Ph | S | H | b10 | H |
| 2-20-1212 | N-Ph | S | t-bu | b10 | H |
| 2-20-1213 | N-Ph | S | methyl | b10 | H |
| 2-20-1214 | N-Ph | S | b1 | b10 | H |
| 2-20-1215 | N-Ph | S | b2 | b10 | H |
| 2-20-1216 | N-Ph | S | b3 | b10 | H |
| 2-20-1217 | N-Ph | S | H | a1 | a1 |
| 2-20-1218 | N-Ph | S | t-bu | a1 | a1 |
| 2-20-1219 | N-Ph | S | methyl | a1 | a1 |
| 2-20-1220 | N-Ph | S | b1 | a1 | a1 |
| 2-20-1221 | N-Ph | S | b2 | a1 | a1 |
| 2-20-1222 | N-Ph | S | b3 | a1 | a1 |
| 2-20-1223 | N-Ph | S | H | a2 | a2 |
| 2-20-1224 | N-Ph | S | t-bu | a2 | a2 |
| 2-20-1225 | N-Ph | S | methyl | a2 | a2 |
| 2-20-1226 | N-Ph | S | b1 | a2 | a2 |
| 2-20-1227 | N-Ph | S | b2 | a2 | a2 |
| 2-20-1228 | N-Ph | S | b3 | a2 | a2 |
| 2-20-1229 | N-Ph | S | H | a3 | a3 |
| 2-20-1230 | N-Ph | S | t-bu | a3 | a3 |
| 2-20-1231 | N-Ph | S | methyl | a3 | a3 |
| 2-20-1232 | N-Ph | S | b1 | a3 | a3 |
| 2-20-1233 | N-Ph | S | b2 | a3 | a3 |
| 2-20-1234 | N-Ph | S | b3 | a3 | a3 |
| 2-20-1235 | N-Ph | S | H | b1 | b1 |
| 2-20-1236 | N-Ph | S | t-bu | b1 | b1 |
| 2-20-1237 | N-Ph | S | methyl | b1 | b1 |
| 2-20-1238 | N-Ph | S | b1 | b1 | b1 |
| 2-20-1239 | N-Ph | S | b2 | b1 | b1 |
| 2-20-1240 | N-Ph | S | b3 | b1 | b1 |
| 2-20-1241 | N-Ph | S | H | b2 | b2 |
| 2-20-1242 | N-Ph | S | t-bu | b2 | b2 |
| 2-20-1243 | N-Ph | S | methyl | b2 | b2 |
| 2-20-1244 | N-Ph | S | b1 | b2 | b2 |
| 2-20-1245 | N-Ph | S | b2 | b2 | b2 |
| 2-20-1246 | N-Ph | S | b3 | b2 | b2 |
| 2-20-1247 | N-Ph | S | H | b3 | b3 |
| 2-20-1248 | N-Ph | S | t-bu | b3 | b3 |
| 2-20-1249 | N-Ph | S | methyl | b3 | b3 |
| 2-20-1250 | N-Ph | S | b1 | b3 | b3 |
| 2-20-1251 | N-Ph | S | b2 | b3 | b3 |
| 2-20-1252 | N-Ph | S | b3 | b3 | b3 |
| 2-20-1253 | N-Ph | S | H | b5 | b5 |
| 2-20-1254 | N-Ph | S | t-bu | b5 | b5 |
| 2-20-1255 | N-Ph | S | methyl | b5 | b5 |
| 2-20-1256 | N-Ph | S | b1 | b5 | b5 |
| 2-20-1257 | N-Ph | S | b2 | b5 | b5 |
| 2-20-1258 | N-Ph | S | b3 | b5 | b5 |
| 2-20-1259 | N-Ph | S | H | b6 | b6 |
| 2-20-1260 | N-Ph | S | t-bu | b6 | b6 |
| 2-20-1261 | N-Ph | S | methyl | b6 | b6 |
| 2-20-1262 | N-Ph | S | b1 | b6 | b6 |
| 2-20-1263 | N-Ph | S | b2 | b6 | b6 |
| 2-20-1264 | N-Ph | S | b3 | b6 | b6 |
| 2-20-1265 | N-Ph | S | H | b7 | b7 |
| 2-20-1266 | N-Ph | S | t-bu | b7 | b7 |
| 2-20-1267 | N-Ph | S | methyl | b7 | b7 |
| 2-20-1268 | N-Ph | S | b1 | b7 | b7 |
| 2-20-1269 | N-Ph | S | b2 | b7 | b7 |
| 2-20-1270 | N-Ph | S | b3 | b7 | b7 |

### Group 2-21

The compounds are represented by the formula 2-21 below, wherein CY₃ is a benzene group, X₁ is B, and Y1, Y2, Y3, R1b, R2b, (R3)a3 are each shown in the table 21 below,

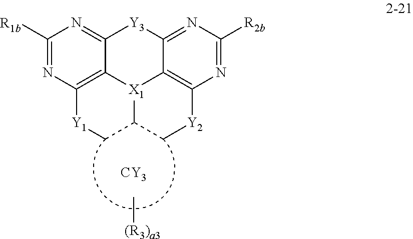

TABLE 21

| Compound | | | | | |
|---|---|---|---|---|---|
| No. | Y1/Y2 | Y3 | R1b | R2b | (R3)a3 |
| 2-21-001 | N-Ph | N-Ph | H | H | H |
| 2-21-002 | N-Ph | N-Ph | t-bu | t-bu | H |
| 2-21-003 | N-Ph | N-Ph | methyl | methyl | H |
| 2-21-004 | N-Ph | N-Ph | t-bu | t-bu | t-bu |
| 2-21-005 | N-Ph | N-Ph | methyl | methyl | methyl |
| 2-21-006 | N-Ph | N-Ph | b1 | b1 | H |
| 2-21-007 | N-Ph | N-Ph | b2 | b2 | H |
| 2-21-008 | N-Ph | N-Ph | b3 | b3 | H |
| 2-21-009 | N-Ph | N-Ph | H | H | a1 |
| 2-21-010 | N-Ph | N-Ph | t-bu | t-bu | a1 |
| 2-21-011 | N-Ph | N-Ph | methyl | methyl | a1 |
| 2-21-012 | N-Ph | N-Ph | b1 | b1 | a1 |
| 2-21-013 | N-Ph | N-Ph | b2 | b2 | a1 |
| 2-21-014 | N-Ph | N-Ph | b3 | b3 | a1 |
| 2-21-015 | N-Ph | N-Ph | H | H | a2 |
| 2-21-016 | N-Ph | N-Ph | t-bu | t-bu | a2 |
| 2-21-017 | N-Ph | N-Ph | methyl | methyl | a2 |
| 2-21-018 | N-Ph | N-Ph | b1 | b1 | a2 |
| 2-21-019 | N-Ph | N-Ph | b2 | b2 | a2 |
| 2-21-020 | N-Ph | N-Ph | b3 | b3 | a2 |
| 2-21-021 | N-Ph | N-Ph | H | H | a3 |
| 2-21-022 | N-Ph | N-Ph | t-bu | t-bu | a3 |
| 2-21-023 | N-Ph | N-Ph | methyl | methyl | a3 |
| 2-21-024 | N-Ph | N-Ph | b1 | b1 | a3 |
| 2-21-025 | N-Ph | N-Ph | b2 | b2 | a3 |
| 2-21-026 | N-Ph | N-Ph | b3 | b3 | a3 |
| 2-21-027 | N-Ph | N-Ph | H | H | a4 |
| 2-21-028 | N-Ph | N-Ph | t-bu | t-bu | a4 |
| 2-21-029 | N-Ph | N-Ph | methyl | methyl | a4 |
| 2-21-030 | N-Ph | N-Ph | b1 | b1 | a4 |
| 2-21-031 | N-Ph | N-Ph | b2 | b2 | a4 |
| 2-21-032 | N-Ph | N-Ph | b3 | b3 | a4 |
| 2-21-033 | N-Ph | N-Ph | H | H | a5 |
| 2-21-034 | N-Ph | N-Ph | t-bu | t-bu | a5 |
| 2-21-035 | N-Ph | N-Ph | methyl | methyl | a5 |
| 2-21-036 | N-Ph | N-Ph | b1 | b1 | a5 |
| 2-21-037 | N-Ph | N-Ph | b2 | b2 | a5 |
| 2-21-038 | N-Ph | N-Ph | b3 | b3 | a5 |
| 2-21-039 | N-Ph | N-Ph | H | H | a6 |
| 2-21-040 | N-Ph | N-Ph | t-bu | t-bu | a6 |
| 2-21-041 | N-Ph | N-Ph | methyl | methyl | a6 |
| 2-21-042 | N-Ph | N-Ph | b1 | b1 | a6 |
| 2-21-043 | N-Ph | N-Ph | b2 | b2 | a6 |
| 2-21-044 | N-Ph | N-Ph | b3 | b3 | a6 |
| 2-21-045 | N-Ph | N-Ph | H | H | b1 |
| 2-21-046 | N-Ph | N-Ph | t-bu | t-bu | b1 |
| 2-21-047 | N-Ph | N-Ph | methyl | methyl | b1 |
| 2-21-048 | N-Ph | N-Ph | b1 | b1 | b1 |
| 2-21-049 | N-Ph | N-Ph | b2 | b2 | b1 |
| 2-21-050 | N-Ph | N-Ph | b3 | b3 | b1 |
| 2-21-051 | N-Ph | N-Ph | H | H | b2 |
| 2-21-052 | N-Ph | N-Ph | t-bu | t-bu | b2 |
| 2-21-053 | N-Ph | N-Ph | methyl | methyl | b2 |
| 2-21-054 | N-Ph | N-Ph | b1 | b1 | b2 |
| 2-21-055 | N-Ph | N-Ph | b2 | b2 | b2 |
| 2-21-056 | N-Ph | N-Ph | b3 | b3 | b2 |
| 2-21-057 | N-Ph | N-Ph | H | H | b3 |
| 2-21-058 | N-Ph | N-Ph | t-bu | t-bu | b3 |
| 2-21-059 | N-Ph | N-Ph | methyl | methyl | b3 |
| 2-21-060 | N-Ph | N-Ph | b1 | b1 | b3 |
| 2-21-061 | N-Ph | N-Ph | b2 | b2 | b3 |
| 2-21-062 | N-Ph | N-Ph | b3 | b3 | b3 |
| 2-21-063 | N-Ph | N-Ph | H | H | b4 |
| 2-21-064 | N-Ph | N-Ph | t-bu | t-bu | b4 |
| 2-21-065 | N-Ph | N-Ph | methyl | methyl | b4 |
| 2-21-066 | N-Ph | N-Ph | b1 | b1 | b4 |
| 2-21-067 | N-Ph | N-Ph | b2 | b2 | b4 |
| 2-21-068 | N-Ph | N-Ph | b3 | b3 | b4 |
| 2-21-069 | N-Ph | N-Ph | H | H | b5 |
| 2-21-070 | N-Ph | N-Ph | t-bu | t-bu | b5 |
| 2-21-071 | N-Ph | N-Ph | methyl | methyl | b5 |
| 2-21-072 | N-Ph | N-Ph | b1 | b1 | b5 |
| 2-21-073 | N-Ph | N-Ph | b2 | b2 | b5 |
| 2-21-074 | N-Ph | N-Ph | b3 | b3 | b5 |
| 2-21-075 | N-Ph | N-Ph | H | H | b6 |
| 2-21-076 | N-Ph | N-Ph | t-bu | t-bu | b6 |
| 2-21-077 | N-Ph | N-Ph | methyl | methyl | b6 |
| 2-21-078 | N-Ph | N-Ph | b1 | b1 | b6 |
| 2-21-079 | N-Ph | N-Ph | b2 | b2 | b6 |
| 2-21-080 | N-Ph | N-Ph | b3 | b3 | b6 |
| 2-21-081 | N-Ph | N-Ph | H | H | b7 |
| 2-21-082 | N-Ph | N-Ph | t-bu | t-bu | b7 |
| 2-21-083 | N-Ph | N-Ph | methyl | methyl | b7 |
| 2-21-084 | N-Ph | N-Ph | b1 | b1 | b7 |
| 2-21-085 | N-Ph | N-Ph | b2 | b2 | b7 |
| 2-21-086 | N-Ph | N-Ph | b3 | b3 | b7 |
| 2-21-087 | N-Ph | N-Ph | H | H | b8 |
| 2-21-088 | N-Ph | N-Ph | t-bu | t-bu | b8 |
| 2-21-089 | N-Ph | N-Ph | methyl | methyl | b8 |
| 2-21-090 | N-Ph | N-Ph | b1 | b1 | b8 |
| 2-21-091 | N-Ph | N-Ph | b2 | b2 | b8 |
| 2-21-092 | N-Ph | N-Ph | b3 | b3 | b8 |
| 2-21-093 | N-Ph | N-Ph | H | H | b9 |
| 2-21-094 | N-Ph | N-Ph | t-bu | t-bu | b9 |
| 2-21-095 | N-Ph | N-Ph | methyl | methyl | b9 |
| 2-21-096 | N-Ph | N-Ph | b1 | b1 | b9 |
| 2-21-097 | N-Ph | N-Ph | b2 | b2 | b9 |
| 2-21-098 | N-Ph | N-Ph | b3 | b3 | b9 |
| 2-21-099 | N-Ph | N-Ph | H | H | b10 |
| 2-21-100 | N-Ph | N-Ph | t-bu | t-bu | b10 |
| 2-21-101 | N-Ph | N-Ph | methyl | methyl | b10 |
| 2-21-102 | N-Ph | N-Ph | b1 | b1 | b10 |
| 2-21-103 | N-Ph | N-Ph | b2 | b2 | b10 |
| 2-21-104 | N-Ph | N-Ph | b3 | b3 | b10 |
| 2-21-105 | N-Ph | N-Ph | H | H | H |
| 2-21-106 | N-Ph | N-Ph | t-bu | t-bu | H |
| 2-21-107 | N-Ph | N-Ph | methyl | methyl | H |
| 2-21-108 | N-Ph | N-Ph | b1 | b1 | H |
| 2-21-109 | N-Ph | N-Ph | b2 | b2 | H |
| 2-21-110 | N-Ph | N-Ph | b3 | b3 | H |
| 2-21-111 | N-Ph | N-Ph | H | H | H |
| 2-21-112 | N-Ph | N-Ph | t-bu | t-bu | H |
| 2-21-113 | N-Ph | N-Ph | methyl | methyl | H |
| 2-21-114 | N-Ph | N-Ph | b1 | b1 | H |
| 2-21-115 | N-Ph | N-Ph | b2 | b2 | H |
| 2-21-116 | N-Ph | N-Ph | b3 | b3 | H |
| 2-21-117 | N-Ph | N-Ph | H | H | H |
| 2-21-118 | N-Ph | N-Ph | t-bu | t-bu | H |
| 2-21-119 | N-Ph | N-Ph | methyl | methyl | H |
| 2-21-120 | N-Ph | N-Ph | b1 | b1 | H |
| 2-21-121 | N-Ph | N-Ph | b2 | b2 | H |
| 2-21-122 | N-Ph | N-Ph | b3 | b3 | H |
| 2-21-123 | N-Ph | N-Ph | H | H | H |
| 2-21-124 | N-Ph | N-Ph | t-bu | t-bu | H |
| 2-21-125 | N-Ph | N-Ph | methyl | methyl | H |
| 2-21-126 | N-Ph | N-Ph | b1 | b1 | H |
| 2-21-127 | N-Ph | N-Ph | b2 | b2 | H |
| 2-21-128 | N-Ph | N-Ph | b3 | b3 | H |
| 2-21-129 | N-Ph | N-Ph | H | H | H |
| 2-21-130 | N-Ph | N-Ph | t-bu | t-bu | H |
| 2-21-131 | N-Ph | N-Ph | methyl | methyl | H |
| 2-21-132 | N-Ph | N-Ph | b1 | b1 | H |
| 2-21-133 | N-Ph | N-Ph | b2 | b2 | H |
| 2-21-134 | N-Ph | N-Ph | b3 | b3 | H |
| 2-21-135 | N-Ph | N-Ph | H | H | H |
| 2-21-136 | N-Ph | N-Ph | t-bu | t-bu | H |
| 2-21-137 | N-Ph | N-Ph | methyl | methyl | H |
| 2-21-138 | N-Ph | N-Ph | b1 | b1 | H |
| 2-21-139 | N-Ph | N-Ph | b2 | b2 | H |
| 2-21-140 | N-Ph | N-Ph | b3 | b3 | H |
| 2-21-141 | N-Ph | N-Ph | H | H | H |
| 2-21-142 | N-Ph | N-Ph | t-bu | t-bu | H |
| 2-21-143 | N-Ph | N-Ph | methyl | methyl | H |
| 2-21-144 | N-Ph | N-Ph | b1 | b1 | H |
| 2-21-145 | N-Ph | N-Ph | b2 | b2 | H |
| 2-21-146 | N-Ph | N-Ph | b3 | b3 | H |
| 2-21-147 | N-Ph | N-Ph | H | H | H |
| 2-21-148 | N-Ph | N-Ph | t-bu | t-bu | H |
| 2-21-149 | N-Ph | N-Ph | methyl | methyl | H |
| 2-21-150 | N-Ph | N-Ph | b1 | b1 | H |
| 2-21-151 | N-Ph | N-Ph | b2 | b2 | H |
| 2-21-152 | N-Ph | N-Ph | b3 | b3 | H |
| 2-21-153 | N-Ph | N-Ph | H | H | H |
| 2-21-154 | N-Ph | N-Ph | t-bu | t-bu | H |
| 2-21-155 | N-Ph | N-Ph | methyl | methyl | H |
| 2-21-156 | N-Ph | N-Ph | b1 | b1 | H |
| 2-21-157 | N-Ph | N-Ph | b2 | b2 | H |
| 2-21-158 | N-Ph | N-Ph | b3 | b3 | H |
| 2-21-159 | N-Ph | N-Ph | H | H | H |
| 2-21-160 | N-Ph | N-Ph | t-bu | t-bu | H |
| 2-21-161 | N-Ph | N-Ph | methyl | methyl | H |
| 2-21-162 | N-Ph | N-Ph | b1 | b1 | H |
| 2-21-163 | N-Ph | N-Ph | b2 | b2 | H |
| 2-21-164 | N-Ph | N-Ph | b3 | b3 | H |
| 2-21-165 | N-Ph | N-Ph | H | H | H |
| 2-21-166 | N-Ph | N-Ph | t-bu | t-bu | H |
| 2-21-167 | N-Ph | N-Ph | methyl | methyl | H |
| 2-21-168 | N-Ph | N-Ph | b1 | b1 | H |
| 2-21-169 | N-Ph | N-Ph | b2 | b2 | H |
| 2-21-170 | N-Ph | N-Ph | b3 | b3 | H |
| 2-21-171 | N-Ph | N-Ph | H | H | H |
| 2-21-172 | N-Ph | N-Ph | t-bu | t-bu | H |
| 2-21-173 | N-Ph | N-Ph | methyl | methyl | H |
| 2-21-174 | N-Ph | N-Ph | b1 | b1 | H |
| 2-21-175 | N-Ph | N-Ph | b2 | b2 | H |
| 2-21-176 | N-Ph | N-Ph | b3 | b3 | H |
| 2-21-177 | N-Ph | N-Ph | H | H | H |
| 2-21-178 | N-Ph | N-Ph | t-bu | t-bu | H |
| 2-21-179 | N-Ph | N-Ph | methyl | methyl | H |
| 2-21-180 | N-Ph | N-Ph | b1 | b1 | H |
| 2-21-181 | N-Ph | N-Ph | b2 | b2 | H |
| 2-21-182 | N-Ph | N-Ph | b3 | b3 | H |
| 2-21-183 | N-Ph | N-Ph | H | H | H |
| 2-21-184 | N-Ph | N-Ph | t-bu | t-bu | H |
| 2-21-185 | N-Ph | N-Ph | methyl | methyl | H |
| 2-21-186 | N-Ph | N-Ph | b1 | b1 | H |
| 2-21-187 | N-Ph | N-Ph | b2 | b2 | H |
| 2-21-188 | N-Ph | N-Ph | b3 | b3 | H |
| 2-21-189 | N-Ph | N-Ph | H | H | H |
| 2-21-190 | N-Ph | N-Ph | t-bu | t-bu | H |
| 2-21-191 | N-Ph | N-Ph | methyl | methyl | H |
| 2-21-192 | N-Ph | N-Ph | b1 | b1 | H |
| 2-21-193 | N-Ph | N-Ph | b2 | b2 | H |
| 2-21-194 | N-Ph | N-Ph | b3 | b3 | H |
| 2-21-195 | N-Ph | N-Ph | H | H | H |
| 2-21-196 | N-Ph | N-Ph | t-bu | t-bu | H |
| 2-21-197 | N-Ph | N-Ph | methyl | methyl | H |
| 2-21-198 | N-Ph | N-Ph | b1 | b1 | H |
| 2-21-199 | N-Ph | N-Ph | b2 | b2 | H |
| 2-21-200 | N-Ph | N-Ph | b3 | b3 | H |
| 2-21-201 | N-Ph | N-Ph | H | H | a1 |
| 2-21-202 | N-Ph | N-Ph | t-bu | t-bu | a1 |
| 2-21-203 | N-Ph | N-Ph | methyl | methyl | a1 |
| 2-21-204 | N-Ph | N-Ph | b1 | b1 | a1 |
| 2-21-205 | N-Ph | N-Ph | b2 | b2 | a1 |
| 2-21-206 | N-Ph | N-Ph | b3 | b3 | a1 |
| 2-21-207 | N-Ph | N-Ph | H | H | a2 |
| 2-21-208 | N-Ph | N-Ph | t-bu | t-bu | a2 |
| 2-21-209 | N-Ph | N-Ph | methyl | methyl | a2 |
| 2-21-210 | N-Ph | N-Ph | b1 | b1 | a2 |
| 2-21-211 | N-Ph | N-Ph | b2 | b2 | a2 |
| 2-21-212 | N-Ph | N-Ph | b3 | b3 | a2 |
| 2-21-213 | N-Ph | N-Ph | H | H | a3 |
| 2-21-214 | N-Ph | N-Ph | t-bu | t-bu | a3 |
| 2-21-215 | N-Ph | N-Ph | methyl | methyl | a3 |
| 2-21-216 | N-Ph | N-Ph | b1 | b1 | a3 |
| 2-21-217 | N-Ph | N-Ph | b2 | b2 | a3 |
| 2-21-218 | N-Ph | N-Ph | b3 | b3 | a3 |
| 2-21-219 | N-Ph | N-Ph | H | H | b1 |
| 2-21-220 | N-Ph | N-Ph | t-bu | t-bu | b1 |
| 2-21-221 | N-Ph | N-Ph | methyl | methyl | b1 |
| 2-21-222 | N-Ph | N-Ph | b1 | b1 | b1 |
| 2-21-223 | N-Ph | N-Ph | b2 | b2 | b1 |
| 2-21-224 | N-Ph | N-Ph | b3 | b3 | b1 |
| 2-21-225 | N-Ph | N-Ph | H | H | b2 |
| 2-21-226 | N-Ph | N-Ph | t-bu | t-bu | b2 |
| 2-21-227 | N-Ph | N-Ph | methyl | methyl | b2 |
| 2-21-228 | N-Ph | N-Ph | b1 | b1 | b2 |
| 2-21-229 | N-Ph | N-Ph | b2 | b2 | b2 |
| 2-21-230 | N-Ph | N-Ph | b3 | b3 | b2 |
| 2-21-231 | N-Ph | N-Ph | H | H | b3 |
| 2-21-232 | N-Ph | N-Ph | t-bu | t-bu | b3 |
| 2-21-233 | N-Ph | N-Ph | methyl | methyl | b3 |
| 2-21-234 | N-Ph | N-Ph | b1 | b1 | b3 |
| 2-21-235 | N-Ph | N-Ph | b2 | b2 | b3 |
| 2-21-236 | N-Ph | N-Ph | b3 | b3 | b3 |
| 2-21-237 | N-Ph | N-Ph | H | H | b5 |
| 2-21-238 | N-Ph | N-Ph | t-bu | t-bu | b5 |
| 2-21-239 | N-Ph | N-Ph | methyl | methyl | b5 |
| 2-21-240 | N-Ph | N-Ph | b1 | b1 | b5 |
| 2-21-241 | N-Ph | N-Ph | b2 | b2 | b5 |
| 2-21-242 | N-Ph | N-Ph | b3 | b3 | b5 |
| 2-21-243 | N-Ph | N-Ph | H | H | b6 |
| 2-21-244 | N-Ph | N-Ph | t-bu | t-bu | b6 |
| 2-21-245 | N-Ph | N-Ph | methyl | methyl | b6 |
| 2-21-246 | N-Ph | N-Ph | b1 | b1 | b6 |
| 2-21-247 | N-Ph | N-Ph | b2 | b2 | b6 |
| 2-21-248 | N-Ph | N-Ph | b3 | b3 | b6 |
| 2-21-249 | N-Ph | N-Ph | H | H | b7 |
| 2-21-250 | N-Ph | N-Ph | t-bu | t-bu | b7 |
| 2-21-251 | N-Ph | N-Ph | methyl | methyl | b7 |
| 2-21-252 | N-Ph | N-Ph | b1 | b1 | b7 |
| 2-21-253 | N-Ph | N-Ph | b2 | b2 | b7 |
| 2-21-254 | N-Ph | N-Ph | b3 | b3 | b7 |
| 2-21-255 | N-tolyl | N-tolyl | H | H | H |
| 2-21-256 | N-tolyl | N-tolyl | t-bu | t-bu | H |
| 2-21-257 | N-tolyl | N-tolyl | methyl | methyl | H |
| 2-21-258 | N-tolyl | N-tolyl | t-bu | t-bu | t-bu |
| 2-21-259 | N-tolyl | N-tolyl | methyl | methyl | methyl |
| 2-21-260 | N-tolyl | N-tolyl | b1 | b1 | H |
| 2-21-261 | N-tolyl | N-tolyl | b2 | b2 | H |
| 2-21-262 | N-tolyl | N-tolyl | b3 | b3 | H |
| 2-21-263 | N-tolyl | N-tolyl | H | H | a1 |
| 2-21-264 | N-tolyl | N-tolyl | t-bu | t-bu | a1 |
| 2-21-265 | N-tolyl | N-tolyl | methyl | methyl | a1 |
| 2-21-266 | N-tolyl | N-tolyl | b1 | b1 | a1 |
| 2-21-267 | N-tolyl | N-tolyl | b2 | b2 | a1 |
| 2-21-268 | N-tolyl | N-tolyl | b3 | b3 | a1 |
| 2-21-269 | N-tolyl | N-tolyl | H | H | a2 |
| 2-21-270 | N-tolyl | N-tolyl | t-bu | t-bu | a2 |
| 2-21-271 | N-tolyl | N-tolyl | methyl | methyl | a2 |
| 2-21-272 | N-tolyl | N-tolyl | b1 | b1 | a2 |
| 2-21-273 | N-tolyl | N-tolyl | b2 | b2 | a2 |
| 2-21-274 | N-tolyl | N-tolyl | b3 | b3 | a2 |
| 2-21-275 | N-tolyl | N-tolyl | H | H | a3 |
| 2-21-276 | N-tolyl | N-tolyl | t-bu | t-bu | a3 |
| 2-21-277 | N-tolyl | N-tolyl | methyl | methyl | a3 |
| 2-21-278 | N-tolyl | N-tolyl | b1 | b1 | a3 |
| 2-21-279 | N-tolyl | N-tolyl | b2 | b2 | a3 |
| 2-21-280 | N-tolyl | N-tolyl | b3 | b3 | a3 |
| 2-21-281 | N-tolyl | N-tolyl | H | H | a4 |
| 2-21-282 | N-tolyl | N-tolyl | t-bu | t-bu | a4 |
| 2-21-283 | N-tolyl | N-tolyl | methyl | methyl | a4 |
| 2-21-284 | N-tolyl | N-tolyl | b1 | b1 | a4 |
| 2-21-285 | N-tolyl | N-tolyl | b2 | b2 | a4 |
| 2-21-286 | N-tolyl | N-tolyl | b3 | b3 | a4 |
| 2-21-287 | N-tolyl | N-tolyl | H | H | a5 |
| 2-21-288 | N-tolyl | N-tolyl | t-bu | t-bu | a5 |
| 2-21-289 | N-tolyl | N-tolyl | methyl | methyl | a5 |
| 2-21-290 | N-tolyl | N-tolyl | b1 | b1 | a5 |
| 2-21-291 | N-tolyl | N-tolyl | b2 | b2 | a5 |
| 2-21-292 | N-tolyl | N-tolyl | b3 | b3 | a5 |
| 2-21-293 | N-tolyl | N-tolyl | H | H | a6 |
| 2-21-294 | N-tolyl | N-tolyl | t-bu | t-bu | a6 |
| 2-21-295 | N-tolyl | N-tolyl | methyl | methyl | a6 |
| 2-21-296 | N-tolyl | N-tolyl | b1 | b1 | a6 |
| 2-21-297 | N-tolyl | N-tolyl | b2 | b2 | a6 |
| 2-21-298 | N-tolyl | N-tolyl | b3 | b3 | a6 |
| 2-21-299 | N-tolyl | N-tolyl | H | H | b1 |
| 2-21-300 | N-tolyl | N-tolyl | t-bu | t-bu | b1 |
| 2-21-301 | N-tolyl | N-tolyl | methyl | methyl | b1 |
| 2-21-302 | N-tolyl | N-tolyl | b1 | b1 | b1 |
| 2-21-303 | N-tolyl | N-tolyl | b2 | b2 | b1 |
| 2-21-304 | N-tolyl | N-tolyl | b3 | b3 | b1 |
| 2-21-305 | N-tolyl | N-tolyl | H | H | b2 |
| 2-21-306 | N-tolyl | N-tolyl | t-bu | t-bu | b2 |
| 2-21-307 | N-tolyl | N-tolyl | methyl | methyl | b2 |
| 2-21-308 | N-tolyl | N-tolyl | b1 | b1 | b2 |
| 2-21-309 | N-tolyl | N-tolyl | b2 | b2 | b2 |
| 2-21-310 | N-tolyl | N-tolyl | b3 | b3 | b2 |
| 2-21-311 | N-tolyl | N-tolyl | H | H | b3 |
| 2-21-312 | N-tolyl | N-tolyl | t-bu | t-bu | b3 |
| 2-21-313 | N-tolyl | N-tolyl | methyl | methyl | b3 |
| 2-21-314 | N-tolyl | N-tolyl | b1 | b1 | b3 |
| 2-21-315 | N-tolyl | N-tolyl | b2 | b2 | b3 |
| 2-21-316 | N-tolyl | N-tolyl | b3 | b3 | b3 |
| 2-21-317 | N-tolyl | N-tolyl | H | H | b4 |
| 2-21-318 | N-tolyl | N-tolyl | t-bu | t-bu | b4 |
| 2-21-319 | N-tolyl | N-tolyl | methyl | methyl | b4 |
| 2-21-320 | N-tolyl | N-tolyl | b1 | b1 | b4 |
| 2-21-321 | N-tolyl | N-tolyl | b2 | b2 | b4 |
| 2-21-322 | N-tolyl | N-tolyl | b3 | b3 | b4 |
| 2-21-323 | N-tolyl | N-tolyl | H | H | b5 |
| 2-21-324 | N-tolyl | N-tolyl | t-bu | t-bu | b5 |
| 2-21-325 | N-tolyl | N-tolyl | methyl | methyl | b5 |
| 2-21-326 | N-tolyl | N-tolyl | b1 | b1 | b5 |
| 2-21-327 | N-tolyl | N-tolyl | b2 | b2 | b5 |
| 2-21-328 | N-tolyl | N-tolyl | b3 | b3 | b5 |
| 2-21-329 | N-tolyl | N-tolyl | H | H | b6 |
| 2-21-330 | N-tolyl | N-tolyl | t-bu | t-bu | b6 |
| 2-21-331 | N-tolyl | N-tolyl | methyl | methyl | b6 |
| 2-21-332 | N-tolyl | N-tolyl | b1 | b1 | b6 |
| 2-21-333 | N-tolyl | N-tolyl | b2 | b2 | b6 |
| 2-21-334 | N-tolyl | N-tolyl | b3 | b3 | b6 |
| 2-21-335 | N-tolyl | N-tolyl | H | H | b7 |
| 2-21-336 | N-tolyl | N-tolyl | t-bu | t-bu | b7 |
| 2-21-337 | N-tolyl | N-tolyl | methyl | methyl | b7 |
| 2-21-338 | N-tolyl | N-tolyl | b1 | b1 | b7 |
| 2-21-339 | N-tolyl | N-tolyl | b2 | b2 | b7 |
| 2-21-340 | N-tolyl | N-tolyl | b3 | b3 | b7 |
| 2-21-341 | N-tolyl | N-tolyl | H | H | b8 |
| 2-21-342 | N-tolyl | N-tolyl | t-bu | t-bu | b8 |
| 2-21-343 | N-tolyl | N-tolyl | methyl | methyl | b8 |
| 2-21-344 | N-tolyl | N-tolyl | b1 | b1 | b8 |
| 2-21-345 | N-tolyl | N-tolyl | b2 | b2 | b8 |
| 2-21-346 | N-tolyl | N-tolyl | b3 | b3 | b8 |
| 2-21-347 | N-tolyl | N-tolyl | H | H | b9 |
| 2-21-348 | N-tolyl | N-tolyl | t-bu | t-bu | b9 |
| 2-21-349 | N-tolyl | N-tolyl | methyl | methyl | b9 |
| 2-21-350 | N-tolyl | N-tolyl | b1 | b1 | b9 |
| 2-21-351 | N-tolyl | N-tolyl | b2 | b2 | b9 |
| 2-21-352 | N-tolyl | N-tolyl | b3 | b3 | b9 |
| 2-21-353 | N-tolyl | N-tolyl | H | H | b10 |
| 2-21-354 | N-tolyl | N-tolyl | t-bu | t-bu | b10 |
| 2-21-355 | N-tolyl | N-tolyl | methyl | methyl | b10 |
| 2-21-356 | N-tolyl | N-tolyl | b1 | b1 | b10 |
| 2-21-357 | N-tolyl | N-tolyl | b2 | b2 | b10 |
| 2-21-358 | N-tolyl | N-tolyl | b3 | b3 | b10 |
| 2-21-359 | N-tolyl | N-tolyl | H | H | H |
| 2-21-360 | N-tolyl | N-tolyl | t-bu | t-bu | H |
| 2-21-361 | N-tolyl | N-tolyl | methyl | methyl | H |
| 2-21-362 | N-tolyl | N-tolyl | b1 | b1 | H |
| 2-21-363 | N-tolyl | N-tolyl | b2 | b2 | H |
| 2-21-364 | N-tolyl | N-tolyl | b3 | b3 | H |
| 2-21-365 | N-tolyl | N-tolyl | H | H | H |
| 2-21-366 | N-tolyl | N-tolyl | t-bu | t-bu | H |
| 2-21-367 | N-tolyl | N-tolyl | methyl | methyl | H |
| 2-21-368 | N-tolyl | N-tolyl | b1 | b1 | H |
| 2-21-369 | N-tolyl | N-tolyl | b2 | b2 | H |
| 2-21-370 | N-tolyl | N-tolyl | b3 | b3 | H |
| 2-21-371 | N-tolyl | N-tolyl | H | H | H |
| 2-21-372 | N-tolyl | N-tolyl | t-bu | t-bu | H |
| 2-21-373 | N-tolyl | N-tolyl | methyl | methyl | H |
| 2-21-374 | N-tolyl | N-tolyl | b1 | b1 | H |
| 2-21-375 | N-tolyl | N-tolyl | b2 | b2 | H |
| 2-21-376 | N-tolyl | N-tolyl | b3 | b3 | H |
| 2-21-377 | N-tolyl | N-tolyl | H | H | H |
| 2-21-378 | N-tolyl | N-tolyl | t-bu | t-bu | H |
| 2-21-379 | N-tolyl | N-tolyl | methyl | methyl | H |
| 2-21-380 | N-tolyl | N-tolyl | b1 | b1 | H |
| 2-21-381 | N-tolyl | N-tolyl | b2 | b2 | H |
| 2-21-382 | N-tolyl | N-tolyl | b3 | b3 | H |
| 2-21-383 | N-tolyl | N-tolyl | H | H | H |
| 2-21-384 | N-tolyl | N-tolyl | t-bu | t-bu | H |
| 2-21-385 | N-tolyl | N-tolyl | methyl | methyl | H |
| 2-21-386 | N-tolyl | N-tolyl | b1 | b1 | H |
| 2-21-387 | N-tolyl | N-tolyl | b2 | b2 | H |
| 2-21-388 | N-tolyl | N-tolyl | b3 | b3 | H |
| 2-21-389 | N-tolyl | N-tolyl | H | H | H |
| 2-21-390 | N-tolyl | N-tolyl | t-bu | t-bu | H |
| 2-21-391 | N-tolyl | N-tolyl | methyl | methyl | H |
| 2-21-392 | N-tolyl | N-tolyl | b1 | b1 | H |
| 2-21-393 | N-tolyl | N-tolyl | b2 | b2 | H |
| 2-21-394 | N-tolyl | N-tolyl | b3 | b3 | H |
| 2-21-395 | N-tolyl | N-tolyl | H | H | H |
| 2-21-396 | N-tolyl | N-tolyl | t-bu | t-bu | H |
| 2-21-397 | N-tolyl | N-tolyl | methyl | methyl | H |
| 2-21-398 | N-tolyl | N-tolyl | b1 | b1 | H |
| 2-21-399 | N-tolyl | N-tolyl | b2 | b2 | H |
| 2-21-400 | N-tolyl | N-tolyl | b3 | b3 | H |
| 2-21-401 | N-tolyl | N-tolyl | H | H | H |
| 2-21-402 | N-tolyl | N-tolyl | t-bu | t-bu | H |
| 2-21-403 | N-tolyl | N-tolyl | methyl | methyl | H |
| 2-21-404 | N-tolyl | N-tolyl | b1 | b1 | H |
| 2-21-405 | N-tolyl | N-tolyl | b2 | b2 | H |
| 2-21-406 | N-tolyl | N-tolyl | b3 | b3 | H |
| 2-21-407 | N-tolyl | N-tolyl | H | H | H |
| 2-21-408 | N-tolyl | N-tolyl | t-bu | t-bu | H |
| 2-21-409 | N-tolyl | N-tolyl | methyl | methyl | H |
| 2-21-410 | N-tolyl | N-tolyl | b1 | b1 | H |
| 2-21-411 | N-tolyl | N-tolyl | b2 | b2 | H |
| 2-21-412 | N-tolyl | N-tolyl | b3 | b3 | H |
| 2-21-413 | N-tolyl | N-tolyl | H | H | H |
| 2-21-414 | N-tolyl | N-tolyl | t-bu | t-bu | H |
| 2-21-415 | N-tolyl | N-tolyl | methyl | methyl | H |
| 2-21-416 | N-tolyl | N-tolyl | b1 | b1 | H |
| 2-21-417 | N-tolyl | N-tolyl | b2 | b2 | H |
| 2-21-418 | N-tolyl | N-tolyl | b3 | b3 | H |
| 2-21-419 | N-tolyl | N-tolyl | H | H | H |
| 2-21-420 | N-tolyl | N-tolyl | t-bu | t-bu | H |
| 2-21-421 | N-tolyl | N-tolyl | methyl | methyl | H |
| 2-21-422 | N-tolyl | N-tolyl | b1 | b1 | H |
| 2-21-423 | N-tolyl | N-tolyl | b2 | b2 | H |
| 2-21-424 | N-tolyl | N-tolyl | b3 | b3 | H |
| 2-21-425 | N-tolyl | N-tolyl | H | H | H |
| 2-21-426 | N-tolyl | N-tolyl | t-bu | t-bu | H |
| 2-21-427 | N-tolyl | N-tolyl | methyl | methyl | H |
| 2-21-428 | N-tolyl | N-tolyl | b1 | b1 | H |
| 2-21-429 | N-tolyl | N-tolyl | b2 | b2 | H |
| 2-21-430 | N-tolyl | N-tolyl | b3 | b3 | H |
| 2-21-431 | N-tolyl | N-tolyl | H | H | H |
| 2-21-432 | N-tolyl | N-tolyl | t-bu | t-bu | H |
| 2-21-433 | N-tolyl | N-tolyl | methyl | methyl | H |
| 2-21-434 | N-tolyl | N-tolyl | b1 | b1 | H |
| 2-21-435 | N-tolyl | N-tolyl | b2 | b2 | H |
| 2-21-436 | N-tolyl | N-tolyl | b3 | b3 | H |
| 2-21-437 | N-tolyl | N-tolyl | H | H | H |
| 2-21-438 | N-tolyl | N-tolyl | t-bu | t-bu | H |
| 2-21-439 | N-tolyl | N-tolyl | methyl | methyl | H |
| 2-21-440 | N-tolyl | N-tolyl | b1 | b1 | H |
| 2-21-441 | N-tolyl | N-tolyl | b2 | b2 | H |
| 2-21-442 | N-tolyl | N-tolyl | b3 | b3 | H |
| 2-21-443 | N-tolyl | N-tolyl | H | H | H |
| 2-21-444 | N-tolyl | N-tolyl | t-bu | t-bu | H |
| 2-21-445 | N-tolyl | N-tolyl | methyl | methyl | H |
| 2-21-446 | N-tolyl | N-tolyl | b1 | b1 | H |
| 2-21-447 | N-tolyl | N-tolyl | b2 | b2 | H |
| 2-21-448 | N-tolyl | N-tolyl | b3 | b3 | H |
| 2-21-449 | N-tolyl | N-tolyl | H | H | H |
| 2-21-450 | N-tolyl | N-tolyl | t-bu | t-bu | H |
| 2-21-451 | N-tolyl | N-tolyl | methyl | methyl | H |
| 2-21-452 | N-tolyl | N-tolyl | b1 | b1 | H |
| 2-21-453 | N-tolyl | N-tolyl | b2 | b2 | H |
| 2-21-454 | N-tolyl | N-tolyl | b3 | b3 | H |
| 2-21-455 | N-tolyl | N-tolyl | H | H | a1 |
| 2-21-456 | N-tolyl | N-tolyl | t-bu | t-bu | a1 |
| 2-21-457 | N-tolyl | N-tolyl | methyl | methyl | a1 |
| 2-21-458 | N-tolyl | N-tolyl | b1 | b1 | a1 |
| 2-21-459 | N-tolyl | N-tolyl | b2 | b2 | a1 |
| 2-21-460 | N-tolyl | N-tolyl | b3 | b3 | a1 |
| 2-21-461 | N-tolyl | N-tolyl | H | H | a2 |
| 2-21-462 | N-tolyl | N-tolyl | t-bu | t-bu | a2 |
| 2-21-463 | N-tolyl | N-tolyl | methyl | methyl | a2 |
| 2-21-464 | N-tolyl | N-tolyl | b1 | b1 | a2 |
| 2-21-465 | N-tolyl | N-tolyl | b2 | b2 | a2 |
| 2-21-466 | N-tolyl | N-tolyl | b3 | b3 | a2 |
| 2-21-467 | N-tolyl | N-tolyl | H | H | a3 |
| 2-21-468 | N-tolyl | N-tolyl | t-bu | t-bu | a3 |
| 2-21-469 | N-tolyl | N-tolyl | methyl | methyl | a3 |
| 2-21-470 | N-tolyl | N-tolyl | b1 | b1 | a3 |
| 2-21-471 | N-tolyl | N-tolyl | b2 | b2 | a3 |
| 2-21-472 | N-tolyl | N-tolyl | b3 | b3 | a3 |
| 2-21-473 | N-tolyl | N-tolyl | H | H | b1 |
| 2-21-474 | N-tolyl | N-tolyl | t-bu | t-bu | b1 |
| 2-21-475 | N-tolyl | N-tolyl | methyl | methyl | b1 |
| 2-21-476 | N-tolyl | N-tolyl | b1 | b1 | b1 |
| 2-21-477 | N-tolyl | N-tolyl | b2 | b2 | b1 |
| 2-21-478 | N-tolyl | N-tolyl | b3 | b3 | b1 |
| 2-21-479 | N-tolyl | N-tolyl | H | H | b2 |
| 2-21-480 | N-tolyl | N-tolyl | t-bu | t-bu | b2 |
| 2-21-481 | N-tolyl | N-tolyl | methyl | methyl | b2 |
| 2-21-482 | N-tolyl | N-tolyl | b1 | b1 | b2 |
| 2-21-483 | N-tolyl | N-tolyl | b2 | b2 | b2 |
| 2-21-484 | N-tolyl | N-tolyl | b3 | b3 | b2 |
| 2-21-485 | N-tolyl | N-tolyl | H | H | b3 |
| 2-21-486 | N-tolyl | N-tolyl | t-bu | t-bu | b3 |
| 2-21-487 | N-tolyl | N-tolyl | methyl | methyl | b3 |
| 2-21-488 | N-tolyl | N-tolyl | b1 | b1 | b3 |
| 2-21-489 | N-tolyl | N-tolyl | b2 | b2 | b3 |
| 2-21-490 | N-tolyl | N-tolyl | b3 | b3 | b3 |
| 2-21-491 | N-tolyl | N-tolyl | H | H | b5 |
| 2-21-492 | N-tolyl | N-tolyl | t-bu | t-bu | b5 |
| 2-21-493 | N-tolyl | N-tolyl | methyl | methyl | b5 |
| 2-21-494 | N-tolyl | N-tolyl | b1 | b1 | b5 |
| 2-21-495 | N-tolyl | N-tolyl | b2 | b2 | b5 |
| 2-21-496 | N-tolyl | N-tolyl | b3 | b3 | b5 |
| 2-21-497 | N-tolyl | N-tolyl | H | H | b6 |
| 2-21-498 | N-tolyl | N-tolyl | t-bu | t-bu | b6 |
| 2-21-499 | N-tolyl | N-tolyl | methyl | methyl | b6 |
| 2-21-500 | N-tolyl | N-tolyl | b1 | b1 | b6 |
| 2-21-501 | N-tolyl | N-tolyl | b2 | b2 | b6 |
| 2-21-502 | N-tolyl | N-tolyl | b3 | b3 | b6 |
| 2-21-503 | N-tolyl | N-tolyl | H | H | b7 |
| 2-21-504 | N-tolyl | N-tolyl | t-bu | t-bu | b7 |
| 2-21-505 | N-tolyl | N-tolyl | methyl | methyl | b7 |
| 2-21-506 | N-tolyl | N-tolyl | b1 | b1 | b7 |
| 2-21-507 | N-tolyl | N-tolyl | b2 | b2 | b7 |
| 2-21-508 | N-tolyl | N-tolyl | b3 | b3 | b7 |
| 2-21-509 | N-Biph | N-Biph | H | H | H |
| 2-21-510 | N-Biph | N-Biph | t-bu | t-bu | H |
| 2-21-511 | N-Biph | N-Biph | methyl | methyl | H |
| 2-21-512 | N-Biph | N-Biph | t-bu | t-bu | t-bu |
| 2-21-513 | N-Biph | N-Biph | methyl | methyl | methyl |
| 2-21-514 | N-Biph | N-Biph | b1 | b1 | H |
| 2-21-515 | N-Biph | N-Biph | b2 | b2 | H |
| 2-21-516 | N-Biph | N-Biph | b3 | b3 | H |
| 2-21-517 | N-Biph | N-Biph | H | H | a1 |
| 2-21-518 | N-Biph | N-Biph | t-bu | t-bu | a1 |
| 2-21-519 | N-Biph | N-Biph | methyl | methyl | a1 |
| 2-21-520 | N-Biph | N-Biph | b1 | b1 | a1 |
| 2-21-521 | N-Biph | N-Biph | b2 | b2 | a1 |
| 2-21-522 | N-Biph | N-Biph | b3 | b3 | a1 |
| 2-21-523 | N-Biph | N-Biph | H | H | a2 |
| 2-21-524 | N-Biph | N-Biph | t-bu | t-bu | a2 |
| 2-21-525 | N-Biph | N-Biph | methyl | methyl | a2 |
| 2-21-526 | N-Biph | N-Biph | b1 | b1 | a2 |
| 2-21-527 | N-Biph | N-Biph | b2 | b2 | a2 |
| 2-21-528 | N-Biph | N-Biph | b3 | b3 | a2 |
| 2-21-529 | N-Biph | N-Biph | H | H | a3 |
| 2-21-530 | N-Biph | N-Biph | t-bu | t-bu | a3 |
| 2-21-531 | N-Biph | N-Biph | methyl | methyl | a3 |
| 2-21-532 | N-Biph | N-Biph | b1 | b1 | a3 |
| 2-21-533 | N-Biph | N-Biph | b2 | b2 | a3 |
| 2-21-534 | N-Biph | N-Biph | b3 | b3 | a3 |
| 2-21-535 | N-Biph | N-Biph | H | H | a4 |
| 2-21-536 | N-Biph | N-Biph | t-bu | t-bu | a4 |
| 2-21-537 | N-Biph | N-Biph | methyl | methyl | a4 |
| 2-21-538 | N-Biph | N-Biph | b1 | b1 | a4 |
| 2-21-539 | N-Biph | N-Biph | b2 | b2 | a4 |
| 2-21-540 | N-Biph | N-Biph | b3 | b3 | a4 |
| 2-21-541 | N-Biph | N-Biph | H | H | a5 |
| 2-21-542 | N-Biph | N-Biph | t-bu | t-bu | a5 |
| 2-21-543 | N-Biph | N-Biph | methyl | methyl | a5 |
| 2-21-544 | N-Biph | N-Biph | b1 | b1 | a5 |
| 2-21-545 | N-Biph | N-Biph | b2 | b2 | a5 |
| 2-21-546 | N-Biph | N-Biph | b3 | b3 | a5 |
| 2-21-547 | N-Biph | N-Biph | H | H | a6 |
| 2-21-548 | N-Biph | N-Biph | t-bu | t-bu | a6 |
| 2-21-549 | N-Biph | N-Biph | methyl | methyl | a6 |
| 2-21-550 | N-Biph | N-Biph | b1 | b1 | a6 |
| 2-21-551 | N-Biph | N-Biph | b2 | b2 | a6 |
| 2-21-552 | N-Biph | N-Biph | b3 | b3 | a6 |
| 2-21-553 | N-Biph | N-Biph | H | H | b1 |
| 2-21-554 | N-Biph | N-Biph | t-bu | t-bu | b1 |
| 2-21-555 | N-Biph | N-Biph | methyl | methyl | b1 |
| 2-21-556 | N-Biph | N-Biph | b1 | b1 | b1 |
| 2-21-557 | N-Biph | N-Biph | b2 | b2 | b1 |
| 2-21-558 | N-Biph | N-Biph | b3 | b3 | b1 |
| 2-21-559 | N-Biph | N-Biph | H | H | b2 |
| 2-21-560 | N-Biph | N-Biph | t-bu | t-bu | b2 |
| 2-21-561 | N-Biph | N-Biph | methyl | methyl | b2 |
| 2-21-562 | N-Biph | N-Biph | b1 | b1 | b2 |
| 2-21-563 | N-Biph | N-Biph | b2 | b2 | b2 |
| 2-21-564 | N-Biph | N-Biph | b3 | b3 | b2 |
| 2-21-565 | N-Biph | N-Biph | H | H | b3 |
| 2-21-566 | N-Biph | N-Biph | t-bu | t-bu | b3 |
| 2-21-567 | N-Biph | N-Biph | methyl | methyl | b3 |
| 2-21-568 | N-Biph | N-Biph | b1 | b1 | b3 |
| 2-21-569 | N-Biph | N-Biph | b2 | b2 | b3 |
| 2-21-570 | N-Biph | N-Biph | b3 | b3 | b3 |
| 2-21-571 | N-Biph | N-Biph | H | H | b4 |
| 2-21-572 | N-Biph | N-Biph | t-bu | t-bu | b4 |
| 2-21-573 | N-Biph | N-Biph | methyl | methyl | b4 |
| 2-21-574 | N-Biph | N-Biph | b1 | b1 | b4 |
| 2-21-575 | N-Biph | N-Biph | b2 | b2 | b4 |
| 2-21-576 | N-Biph | N-Biph | b3 | b3 | b4 |
| 2-21-577 | N-Biph | N-Biph | H | H | b5 |
| 2-21-578 | N-Biph | N-Biph | t-bu | t-bu | b5 |
| 2-21-579 | N-Biph | N-Biph | methyl | methyl | b5 |
| 2-21-580 | N-Biph | N-Biph | b1 | b1 | b5 |
| 2-21-581 | N-Biph | N-Biph | b2 | b2 | b5 |
| 2-21-582 | N-Biph | N-Biph | b3 | b3 | b5 |
| 2-21-583 | N-Biph | N-Biph | H | H | b6 |
| 2-21-584 | N-Biph | N-Biph | t-bu | t-bu | b6 |
| 2-21-585 | N-Biph | N-Biph | methyl | methyl | b6 |
| 2-21-586 | N-Biph | N-Biph | b1 | b1 | b6 |
| 2-21-587 | N-Biph | N-Biph | b2 | b2 | b6 |
| 2-21-588 | N-Biph | N-Biph | b3 | b3 | b6 |
| 2-21-589 | N-Biph | N-Biph | H | H | b7 |
| 2-21-590 | N-Biph | N-Biph | t-bu | t-bu | b7 |
| 2-21-591 | N-Biph | N-Biph | methyl | methyl | b7 |
| 2-21-592 | N-Biph | N-Biph | b1 | b1 | b7 |
| 2-21-593 | N-Biph | N-Biph | b2 | b2 | b7 |
| 2-21-594 | N-Biph | N-Biph | b3 | b3 | b7 |
| 2-21-595 | N-Biph | N-Biph | H | H | b8 |
| 2-21-596 | N-Biph | N-Biph | t-bu | t-bu | b8 |
| 2-21-597 | N-Biph | N-Biph | methyl | methyl | b8 |
| 2-21-598 | N-Biph | N-Biph | b1 | b1 | b8 |
| 2-21-599 | N-Biph | N-Biph | b2 | b2 | b8 |
| 2-21-600 | N-Biph | N-Biph | b3 | b3 | b8 |
| 2-21-601 | N-Biph | N-Biph | H | H | b9 |
| 2-21-602 | N-Biph | N-Biph | t-bu | t-bu | b9 |
| 2-21-603 | N-Biph | N-Biph | methyl | methyl | b9 |
| 2-21-604 | N-Biph | N-Biph | b1 | b1 | b9 |
| 2-21-605 | N-Biph | N-Biph | b2 | b2 | b9 |
| 2-21-606 | N-Biph | N-Biph | b3 | b3 | b9 |
| 2-21-607 | N-Biph | N-Biph | H | H | b10 |
| 2-21-608 | N-Biph | N-Biph | t-bu | t-bu | b10 |
| 2-21-609 | N-Biph | N-Biph | methyl | methyl | b10 |
| 2-21-610 | N-Biph | N-Biph | b1 | b1 | b10 |
| 2-21-611 | N-Biph | N-Biph | b2 | b2 | b10 |
| 2-21-612 | N-Biph | N-Biph | b3 | b3 | b10 |
| 2-21-613 | N-Biph | N-Biph | H | H | H |
| 2-21-614 | N-Biph | N-Biph | t-bu | t-bu | H |
| 2-21-615 | N-Biph | N-Biph | methyl | methyl | H |
| 2-21-616 | N-Biph | N-Biph | b1 | b1 | H |
| 2-21-617 | N-Biph | N-Biph | b2 | b2 | H |
| 2-21-618 | N-Biph | N-Biph | b3 | b3 | H |
| 2-21-619 | N-Biph | N-Biph | H | H | H |
| 2-21-620 | N-Biph | N-Biph | t-bu | t-bu | H |
| 2-21-621 | N-Biph | N-Biph | methyl | methyl | H |
| 2-21-622 | N-Biph | N-Biph | b1 | b1 | H |
| 2-21-623 | N-Biph | N-Biph | b2 | b2 | H |
| 2-21-624 | N-Biph | N-Biph | b3 | b3 | H |
| 2-21-625 | N-Biph | N-Biph | H | H | H |
| 2-21-626 | N-Biph | N-Biph | t-bu | t-bu | H |
| 2-21-627 | N-Biph | N-Biph | methyl | methyl | H |
| 2-21-628 | N-Biph | N-Biph | b1 | b1 | H |
| 2-21-629 | N-Biph | N-Biph | b2 | b2 | H |
| 2-21-630 | N-Biph | N-Biph | b3 | b3 | H |
| 2-21-631 | N-Biph | N-Biph | H | H | H |
| 2-21-632 | N-Biph | N-Biph | t-bu | t-bu | H |
| 2-21-633 | N-Biph | N-Biph | methyl | methyl | H |
| 2-21-634 | N-Biph | N-Biph | b1 | b1 | H |
| 2-21-635 | N-Biph | N-Biph | b2 | b2 | H |
| 2-21-636 | N-Biph | N-Biph | b3 | b3 | H |
| 2-21-637 | N-Biph | N-Biph | H | H | H |
| 2-21-638 | N-Biph | N-Biph | t-bu | t-bu | H |
| 2-21-639 | N-Biph | N-Biph | methyl | methyl | H |
| 2-21-640 | N-Biph | N-Biph | b1 | b1 | H |
| 2-21-641 | N-Biph | N-Biph | b2 | b2 | H |
| 2-21-642 | N-Biph | N-Biph | b3 | b3 | H |
| 2-21-643 | N-Biph | N-Biph | H | H | H |
| 2-21-644 | N-Biph | N-Biph | t-bu | t-bu | H |
| 2-21-645 | N-Biph | N-Biph | methyl | methyl | H |
| 2-21-646 | N-Biph | N-Biph | b1 | b1 | H |
| 2-21-647 | N-Biph | N-Biph | b2 | b2 | H |
| 2-21-648 | N-Biph | N-Biph | b3 | b3 | H |
| 2-21-649 | N-Biph | N-Biph | H | H | H |
| 2-21-650 | N-Biph | N-Biph | t-bu | t-bu | H |
| 2-21-651 | N-Biph | N-Biph | methyl | methyl | H |
| 2-21-652 | N-Biph | N-Biph | b1 | b1 | H |
| 2-21-653 | N-Biph | N-Biph | b2 | b2 | H |
| 2-21-654 | N-Biph | N-Biph | b3 | b3 | H |
| 2-21-655 | N-Biph | N-Biph | H | H | H |
| 2-21-656 | N-Biph | N-Biph | t-bu | t-bu | H |
| 2-21-657 | N-Biph | N-Biph | methyl | methyl | H |
| 2-21-658 | N-Biph | N-Biph | b1 | b1 | H |
| 2-21-659 | N-Biph | N-Biph | b2 | b2 | H |
| 2-21-660 | N-Biph | N-Biph | b3 | b3 | H |
| 2-21-661 | N-Biph | N-Biph | H | H | H |
| 2-21-662 | N-Biph | N-Biph | t-bu | t-bu | H |
| 2-21-663 | N-Biph | N-Biph | methyl | methyl | H |
| 2-21-664 | N-Biph | N-Biph | b1 | b1 | H |
| 2-21-665 | N-Biph | N-Biph | b2 | b2 | H |
| 2-21-666 | N-Biph | N-Biph | b3 | b3 | H |
| 2-21-667 | N-Biph | N-Biph | H | H | H |
| 2-21-668 | N-Biph | N-Biph | t-bu | t-bu | H |
| 2-21-669 | N-Biph | N-Biph | methyl | methyl | H |
| 2-21-670 | N-Biph | N-Biph | b1 | b1 | H |
| 2-21-671 | N-Biph | N-Biph | b2 | b2 | H |
| 2-21-672 | N-Biph | N-Biph | b3 | b3 | H |
| 2-21-673 | N-Biph | N-Biph | H | H | H |
| 2-21-674 | N-Biph | N-Biph | t-bu | t-bu | H |
| 2-21-675 | N-Biph | N-Biph | methyl | methyl | H |
| 2-21-676 | N-Biph | N-Biph | b1 | b1 | H |
| 2-21-677 | N-Biph | N-Biph | b2 | b2 | H |
| 2-21-678 | N-Biph | N-Biph | b3 | b3 | H |
| 2-21-679 | N-Biph | N-Biph | H | H | H |
| 2-21-680 | N-Biph | N-Biph | t-bu | t-bu | H |
| 2-21-681 | N-Biph | N-Biph | methyl | methyl | H |
| 2-21-682 | N-Biph | N-Biph | b1 | b1 | H |
| 2-21-683 | N-Biph | N-Biph | b2 | b2 | H |
| 2-21-684 | N-Biph | N-Biph | b3 | b3 | H |
| 2-21-685 | N-Biph | N-Biph | H | H | H |
| 2-21-686 | N-Biph | N-Biph | t-bu | t-bu | H |
| 2-21-687 | N-Biph | N-Biph | methyl | methyl | H |
| 2-21-688 | N-Biph | N-Biph | b1 | b1 | H |
| 2-21-689 | N-Biph | N-Biph | b2 | b2 | H |
| 2-21-690 | N-Biph | N-Biph | b3 | b3 | H |
| 2-21-691 | N-Biph | N-Biph | H | H | H |
| 2-21-692 | N-Biph | N-Biph | t-bu | t-bu | H |
| 2-21-693 | N-Biph | N-Biph | methyl | methyl | H |
| 2-21-694 | N-Biph | N-Biph | b1 | b1 | H |
| 2-21-695 | N-Biph | N-Biph | b2 | b2 | H |
| 2-21-696 | N-Biph | N-Biph | b3 | b3 | H |
| 2-21-697 | N-Biph | N-Biph | H | H | H |
| 2-21-698 | N-Biph | N-Biph | t-bu | t-bu | H |
| 2-21-699 | N-Biph | N-Biph | methyl | methyl | H |
| 2-21-700 | N-Biph | N-Biph | b1 | b1 | H |
| 2-21-701 | N-Biph | N-Biph | b2 | b2 | H |
| 2-21-702 | N-Biph | N-Biph | b3 | b3 | H |
| 2-21-703 | N-Biph | N-Biph | H | H | H |
| 2-21-704 | N-Biph | N-Biph | t-bu | t-bu | H |
| 2-21-705 | N-Biph | N-Biph | methyl | methyl | H |
| 2-21-706 | N-Biph | N-Biph | b1 | b1 | H |
| 2-21-707 | N-Biph | N-Biph | b2 | b2 | H |
| 2-21-708 | N-Biph | N-Biph | b3 | b3 | H |
| 2-21-709 | N-Biph | N-Biph | H | H | a1 |
| 2-21-710 | N-Biph | N-Biph | t-bu | t-bu | a1 |
| 2-21-711 | N-Biph | N-Biph | methyl | methyl | a1 |
| 2-21-712 | N-Biph | N-Biph | b1 | b1 | a1 |
| 2-21-713 | N-Biph | N-Biph | b2 | b2 | a1 |
| 2-21-714 | N-Biph | N-Biph | b3 | b3 | a1 |
| 2-21-715 | N-Biph | N-Biph | H | H | a2 |
| 2-21-716 | N-Biph | N-Biph | t-bu | t-bu | a2 |
| 2-21-717 | N-Biph | N-Biph | methyl | methyl | a2 |
| 2-21-718 | N-Biph | N-Biph | b1 | b1 | a2 |
| 2-21-719 | N-Biph | N-Biph | b2 | b2 | a2 |
| 2-21-720 | N-Biph | N-Biph | b3 | b3 | a2 |
| 2-21-721 | N-Biph | N-Biph | H | H | a3 |
| 2-21-722 | N-Biph | N-Biph | t-bu | t-bu | a3 |
| 2-21-723 | N-Biph | N-Biph | methyl | methyl | a3 |
| 2-21-724 | N-Biph | N-Biph | b1 | b1 | a3 |
| 2-21-725 | N-Biph | N-Biph | b2 | b2 | a3 |
| 2-21-726 | N-Biph | N-Biph | b3 | b3 | a3 |
| 2-21-727 | N-Biph | N-Biph | H | H | b1 |
| 2-21-728 | N-Biph | N-Biph | t-bu | t-bu | b1 |
| 2-21-729 | N-Biph | N-Biph | methyl | methyl | b1 |
| 2-21-730 | N-Biph | N-Biph | b1 | b1 | b1 |
| 2-21-731 | N-Biph | N-Biph | b2 | b2 | b1 |
| 2-21-732 | N-Biph | N-Biph | b3 | b3 | b1 |
| 2-21-733 | N-Biph | N-Biph | H | H | b2 |
| 2-21-734 | N-Biph | N-Biph | t-bu | t-bu | b2 |
| 2-21-735 | N-Biph | N-Biph | methyl | methyl | b2 |
| 2-21-736 | N-Biph | N-Biph | b1 | b1 | b2 |
| 2-21-737 | N-Biph | N-Biph | b2 | b2 | b2 |
| 2-21-738 | N-Biph | N-Biph | b3 | b3 | b2 |
| 2-21-739 | N-Biph | N-Biph | H | H | b3 |
| 2-21-740 | N-Biph | N-Biph | t-bu | t-bu | b3 |
| 2-21-741 | N-Biph | N-Biph | methyl | methyl | b3 |
| 2-21-742 | N-Biph | N-Biph | b1 | b1 | b3 |
| 2-21-743 | N-Biph | N-Biph | b2 | b2 | b3 |
| 2-21-744 | N-Biph | N-Biph | b3 | b3 | b3 |
| 2-21-745 | N-Biph | N-Biph | H | H | b5 |
| 2-21-746 | N-Biph | N-Biph | t-bu | t-bu | b5 |
| 2-21-747 | N-Biph | N-Biph | methyl | methyl | b5 |
| 2-21-748 | N-Biph | N-Biph | b1 | b1 | b5 |
| 2-21-749 | N-Biph | N-Biph | b2 | b2 | b5 |
| 2-21-750 | N-Biph | N-Biph | b3 | b3 | b5 |
| 2-21-751 | N-Biph | N-Biph | H | H | b6 |
| 2-21-752 | N-Biph | N-Biph | t-bu | t-bu | b6 |
| 2-21-753 | N-Biph | N-Biph | methyl | methyl | b6 |
| 2-21-754 | N-Biph | N-Biph | b1 | b1 | b6 |
| 2-21-755 | N-Biph | N-Biph | b2 | b2 | b6 |
| 2-21-756 | N-Biph | N-Biph | b3 | b3 | b6 |
| 2-21-757 | N-Biph | N-Biph | H | H | b7 |
| 2-21-758 | N-Biph | N-Biph | t-bu | t-bu | b7 |
| 2-21-759 | N-Biph | N-Biph | methyl | methyl | b7 |
| 2-21-760 | N-Biph | N-Biph | b1 | b1 | b7 |
| 2-21-761 | N-Biph | N-Biph | b2 | b2 | b7 |
| 2-21-762 | N-Biph | N-Biph | b3 | b3 | b7 |
| 2-21-763 | N-Ph | O | H | H | H |
| 2-21-764 | N-Ph | O | t-bu | t-bu | H |
| 2-21-765 | N-Ph | O | methyl | methyl | H |
| 2-21-766 | N-Ph | O | t-bu | t-bu | t-bu |
| 2-21-767 | N-Ph | O | methyl | methyl | methyl |
| 2-21-768 | N-Ph | O | b1 | b1 | H |
| 2-21-769 | N-Ph | O | b2 | b2 | H |
| 2-21-770 | N-Ph | O | b3 | b3 | H |
| 2-21-771 | N-Ph | O | H | H | a1 |
| 2-21-772 | N-Ph | O | t-bu | t-bu | a1 |
| 2-21-773 | N-Ph | O | methyl | methyl | a1 |
| 2-21-774 | N-Ph | O | b1 | b1 | a1 |
| 2-21-775 | N-Ph | O | b2 | b2 | a1 |
| 2-21-776 | N-Ph | O | b3 | b3 | a1 |
| 2-21-777 | N-Ph | O | H | H | a2 |
| 2-21-778 | N-Ph | O | t-bu | t-bu | a2 |
| 2-21-779 | N-Ph | O | methyl | methyl | a2 |
| 2-21-780 | N-Ph | O | b1 | b1 | a2 |
| 2-21-781 | N-Ph | O | b2 | b2 | a2 |
| 2-21-782 | N-Ph | O | b3 | b3 | a2 |
| 2-21-783 | N-Ph | O | H | H | a3 |
| 2-21-784 | N-Ph | O | t-bu | t-bu | a3 |
| 2-21-785 | N-Ph | O | methyl | methyl | a3 |
| 2-21-786 | N-Ph | O | b1 | b1 | a3 |
| 2-21-787 | N-Ph | O | b2 | b2 | a3 |
| 2-21-788 | N-Ph | O | b3 | b3 | a3 |
| 2-21-789 | N-Ph | O | H | H | a4 |
| 2-21-790 | N-Ph | O | t-bu | t-bu | a4 |
| 2-21-791 | N-Ph | O | methyl | methyl | a4 |
| 2-21-792 | N-Ph | O | b1 | b1 | a4 |
| 2-21-793 | N-Ph | O | b2 | b2 | a4 |
| 2-21-794 | N-Ph | O | b3 | b3 | a4 |
| 2-21-795 | N-Ph | O | H | H | a5 |
| 2-21-796 | N-Ph | O | t-bu | t-bu | a5 |
| 2-21-797 | N-Ph | O | methyl | methyl | a5 |
| 2-21-798 | N-Ph | O | b1 | b1 | a5 |
| 2-21-799 | N-Ph | O | b2 | b2 | a5 |
| 2-21-800 | N-Ph | O | b3 | b3 | a5 |
| 2-21-801 | N-Ph | O | H | H | a6 |
| 2-21-802 | N-Ph | O | t-bu | t-bu | a6 |
| 2-21-803 | N-Ph | O | methyl | methyl | a6 |
| 2-21-804 | N-Ph | O | b1 | b1 | a6 |
| 2-21-805 | N-Ph | O | b2 | b2 | a6 |
| 2-21-806 | N-Ph | O | b3 | b3 | a6 |
| 2-21-807 | N-Ph | O | H | H | b1 |
| 2-21-808 | N-Ph | O | t-bu | t-bu | b1 |
| 2-21-809 | N-Ph | O | methyl | methyl | b1 |
| 2-21-810 | N-Ph | O | b1 | b1 | b1 |
| 2-21-811 | N-Ph | O | b2 | b2 | b1 |
| 2-21-812 | N-Ph | O | b3 | b3 | b1 |
| 2-21-813 | N-Ph | O | H | H | b2 |
| 2-21-814 | N-Ph | O | t-bu | t-bu | b2 |
| 2-21-815 | N-Ph | O | methyl | methyl | b2 |
| 2-21-816 | N-Ph | O | b1 | b1 | b2 |
| 2-21-817 | N-Ph | O | b2 | b2 | b2 |
| 2-21-818 | N-Ph | O | b3 | b3 | b2 |
| 2-21-819 | N-Ph | O | H | H | b3 |
| 2-21-820 | N-Ph | O | t-bu | t-bu | b3 |
| 2-21-821 | N-Ph | O | methyl | methyl | b3 |
| 2-21-822 | N-Ph | O | b1 | b1 | b3 |
| 2-21-823 | N-Ph | O | b2 | b2 | b3 |
| 2-21-824 | N-Ph | O | b3 | b3 | b3 |
| 2-21-825 | N-Ph | O | H | H | b4 |
| 2-21-826 | N-Ph | O | t-bu | t-bu | b4 |
| 2-21-827 | N-Ph | O | methyl | methyl | b4 |
| 2-21-828 | N-Ph | O | b1 | b1 | b4 |
| 2-21-829 | N-Ph | O | b2 | b2 | b4 |
| 2-21-830 | N-Ph | O | b3 | b3 | b4 |
| 2-21-831 | N-Ph | O | H | H | b5 |
| 2-21-832 | N-Ph | O | t-bu | t-bu | b5 |
| 2-21-833 | N-Ph | O | methyl | methyl | b5 |
| 2-21-834 | N-Ph | O | b1 | b1 | b5 |
| 2-21-835 | N-Ph | O | b2 | b2 | b5 |
| 2-21-836 | N-Ph | O | b3 | b3 | b5 |
| 2-21-837 | N-Ph | O | H | H | b6 |
| 2-21-838 | N-Ph | O | t-bu | t-bu | b6 |
| 2-21-839 | N-Ph | O | methyl | methyl | b6 |
| 2-21-840 | N-Ph | O | b1 | b1 | b6 |
| 2-21-841 | N-Ph | O | b2 | b2 | b6 |
| 2-21-842 | N-Ph | O | b3 | b3 | b6 |
| 2-21-843 | N-Ph | O | H | H | b7 |
| 2-21-844 | N-Ph | O | t-bu | t-bu | b7 |
| 2-21-845 | N-Ph | O | methyl | methyl | b7 |
| 2-21-846 | N-Ph | O | b1 | b1 | b7 |
| 2-21-847 | N-Ph | O | b2 | b2 | b7 |
| 2-21-848 | N-Ph | O | b3 | b3 | b7 |
| 2-21-849 | N-Ph | O | H | H | b8 |
| 2-21-850 | N-Ph | O | t-bu | t-bu | b8 |
| 2-21-851 | N-Ph | O | methyl | methyl | b8 |
| 2-21-852 | N-Ph | O | b1 | b1 | b8 |
| 2-21-853 | N-Ph | O | b2 | b2 | b8 |
| 2-21-854 | N-Ph | O | b3 | b3 | b8 |
| 2-21-855 | N-Ph | O | H | H | b9 |
| 2-21-856 | N-Ph | O | t-bu | t-bu | b9 |
| 2-21-857 | N-Ph | O | methyl | methyl | b9 |
| 2-21-858 | N-Ph | O | b1 | b1 | b9 |
| 2-21-859 | N-Ph | O | b2 | b2 | b9 |
| 2-21-860 | N-Ph | O | b3 | b3 | b9 |
| 2-21-861 | N-Ph | O | H | H | b10 |
| 2-21-862 | N-Ph | O | t-bu | t-bu | b10 |
| 2-21-863 | N-Ph | O | methyl | methyl | b10 |
| 2-21-864 | N-Ph | O | b1 | b1 | b10 |
| 2-21-865 | N-Ph | O | b2 | b2 | b10 |
| 2-21-866 | N-Ph | O | b3 | b3 | b10 |
| 2-21-867 | N-Ph | O | H | H | H |
| 2-21-868 | N-Ph | O | t-bu | t-bu | H |
| 2-21-869 | N-Ph | O | methyl | methyl | H |
| 2-21-870 | N-Ph | O | b1 | b1 | H |
| 2-21-871 | N-Ph | O | b2 | b2 | H |
| 2-21-872 | N-Ph | O | b3 | b3 | H |
| 2-21-873 | N-Ph | O | H | H | H |
| 2-21-874 | N-Ph | O | t-bu | t-bu | H |
| 2-21-875 | N-Ph | O | methyl | methyl | H |
| 2-21-876 | N-Ph | O | b1 | b1 | H |
| 2-21-877 | N-Ph | O | b2 | b2 | H |
| 2-21-878 | N-Ph | O | b3 | b3 | H |
| 2-21-879 | N-Ph | O | H | H | H |
| 2-21-880 | N-Ph | O | t-bu | t-bu | H |
| 2-21-881 | N-Ph | O | methyl | methyl | H |
| 2-21-882 | N-Ph | O | b1 | b1 | H |
| 2-21-883 | N-Ph | O | b2 | b2 | H |
| 2-21-884 | N-Ph | O | b3 | b3 | H |
| 2-21-885 | N-Ph | O | H | H | H |
| 2-21-886 | N-Ph | O | t-bu | t-bu | H |
| 2-21-887 | N-Ph | O | methyl | methyl | H |
| 2-21-888 | N-Ph | O | b1 | b1 | H |
| 2-21-889 | N-Ph | O | b2 | b2 | H |
| 2-21-890 | N-Ph | O | b3 | b3 | H |
| 2-21-891 | N-Ph | O | H | H | H |
| 2-21-892 | N-Ph | O | t-bu | t-bu | H |
| 2-21-893 | N-Ph | O | methyl | methyl | H |
| 2-21-894 | N-Ph | O | b1 | b1 | H |
| 2-21-895 | N-Ph | O | b2 | b2 | H |
| 2-21-896 | N-Ph | O | b3 | b3 | H |
| 2-21-897 | N-Ph | O | H | H | H |
| 2-21-898 | N-Ph | O | t-bu | t-bu | H |
| 2-21-899 | N-Ph | O | methyl | methyl | H |
| 2-21-900 | N-Ph | O | b1 | b1 | H |
| 2-21-901 | N-Ph | O | b2 | b2 | H |
| 2-21-902 | N-Ph | O | b3 | b3 | H |
| 2-21-903 | N-Ph | O | H | H | H |
| 2-21-904 | N-Ph | O | t-bu | t-bu | H |
| 2-21-905 | N-Ph | O | methyl | methyl | H |
| 2-21-906 | N-Ph | O | b1 | b1 | H |
| 2-21-907 | N-Ph | O | b2 | b2 | H |
| 2-21-908 | N-Ph | O | b3 | b3 | H |
| 2-21-909 | N-Ph | O | H | H | H |
| 2-21-910 | N-Ph | O | t-bu | t-bu | H |
| 2-21-911 | N-Ph | O | methyl | methyl | H |
| 2-21-912 | N-Ph | O | b1 | b1 | H |
| 2-21-913 | N-Ph | O | b2 | b2 | H |
| 2-21-914 | N-Ph | O | b3 | b3 | H |
| 2-21-915 | N-Ph | O | H | H | H |
| 2-21-916 | N-Ph | O | t-bu | t-bu | H |
| 2-21-917 | N-Ph | O | methyl | methyl | H |
| 2-21-918 | N-Ph | O | b1 | b1 | H |
| 2-21-919 | N-Ph | O | b2 | b2 | H |
| 2-21-920 | N-Ph | O | b3 | b3 | H |
| 2-21-921 | N-Ph | O | H | H | H |
| 2-21-922 | N-Ph | O | t-bu | t-bu | H |
| 2-21-923 | N-Ph | O | methyl | methyl | H |
| 2-21-924 | N-Ph | O | b1 | b1 | H |
| 2-21-925 | N-Ph | O | b2 | b2 | H |
| 2-21-926 | N-Ph | O | b3 | b3 | H |
| 2-21-927 | N-Ph | O | H | H | H |
| 2-21-928 | N-Ph | O | t-bu | t-bu | H |
| 2-21-929 | N-Ph | O | methyl | methyl | H |
| 2-21-930 | N-Ph | O | b1 | b1 | H |
| 2-21-931 | N-Ph | O | b2 | b2 | H |
| 2-21-932 | N-Ph | O | b3 | b3 | H |
| 2-21-933 | N-Ph | O | H | H | H |
| 2-21-934 | N-Ph | O | t-bu | t-bu | H |
| 2-21-935 | N-Ph | O | methyl | methyl | H |
| 2-21-936 | N-Ph | O | b1 | b1 | H |
| 2-21-937 | N-Ph | O | b2 | b2 | H |
| 2-21-938 | N-Ph | O | b3 | b3 | H |
| 2-21-939 | N-Ph | O | H | H | H |
| 2-21-940 | N-Ph | O | t-bu | t-bu | H |
| 2-21-941 | N-Ph | O | methyl | methyl | H |
| 2-21-942 | N-Ph | O | b1 | b1 | H |
| 2-21-943 | N-Ph | O | b2 | b2 | H |
| 2-21-944 | N-Ph | O | b3 | b3 | H |
| 2-21-945 | N-Ph | O | H | H | H |
| 2-21-946 | N-Ph | O | t-bu | t-bu | H |
| 2-21-947 | N-Ph | O | methyl | methyl | H |
| 2-21-948 | N-Ph | O | b1 | b1 | H |
| 2-21-949 | N-Ph | O | b2 | b2 | H |
| 2-21-950 | N-Ph | O | b3 | b3 | H |
| 2-21-951 | N-Ph | O | H | H | H |
| 2-21-952 | N-Ph | O | t-bu | t-bu | H |
| 2-21-953 | N-Ph | O | methyl | methyl | H |
| 2-21-954 | N-Ph | O | b1 | b1 | H |
| 2-21-955 | N-Ph | O | b2 | b2 | H |
| 2-21-956 | N-Ph | O | b3 | b3 | H |
| 2-21-957 | N-Ph | O | H | H | H |
| 2-21-958 | N-Ph | O | t-bu | t-bu | H |
| 2-21-959 | N-Ph | O | methyl | methyl | H |
| 2-21-960 | N-Ph | O | b1 | b1 | H |
| 2-21-961 | N-Ph | O | b2 | b2 | H |
| 2-21-962 | N-Ph | O | b3 | b3 | H |
| 2-21-963 | N-Ph | O | H | H | a1 |
| 2-21-964 | N-Ph | O | t-bu | t-bu | a1 |
| 2-21-965 | N-Ph | O | methyl | methyl | a1 |
| 2-21-966 | N-Ph | O | b1 | b1 | a1 |
| 2-21-967 | N-Ph | O | b2 | b2 | a1 |
| 2-21-968 | N-Ph | O | b3 | b3 | a1 |
| 2-21-969 | N-Ph | O | H | H | a2 |
| 2-21-970 | N-Ph | O | t-bu | t-bu | a2 |
| 2-21-971 | N-Ph | O | methyl | methyl | a2 |
| 2-21-972 | N-Ph | O | b1 | b1 | a2 |
| 2-21-973 | N-Ph | O | b2 | b2 | a2 |
| 2-21-974 | N-Ph | O | b3 | b3 | a2 |
| 2-21-975 | N-Ph | O | H | H | a3 |
| 2-21-976 | N-Ph | O | t-bu | t-bu | a3 |
| 2-21-977 | N-Ph | O | methyl | methyl | a3 |
| 2-21-978 | N-Ph | O | b1 | b1 | a3 |
| 2-21-979 | N-Ph | O | b2 | b2 | a3 |
| 2-21-980 | N-Ph | O | b3 | b3 | a3 |
| 2-21-981 | N-Ph | O | H | H | b1 |
| 2-21-982 | N-Ph | O | t-bu | t-bu | b1 |
| 2-21-983 | N-Ph | O | methyl | methyl | b1 |
| 2-21-984 | N-Ph | O | b1 | b1 | b1 |
| 2-21-985 | N-Ph | O | b2 | b2 | b1 |
| 2-21-986 | N-Ph | O | b3 | b3 | b1 |
| 2-21-987 | N-Ph | O | H | H | b2 |
| 2-21-988 | N-Ph | O | t-bu | t-bu | b2 |
| 2-21-989 | N-Ph | O | methyl | methyl | b2 |
| 2-21-990 | N-Ph | O | b1 | b1 | b2 |
| 2-21-991 | N-Ph | O | b2 | b2 | b2 |
| 2-21-992 | N-Ph | O | b3 | b3 | b2 |
| 2-21-993 | N-Ph | O | H | H | b3 |
| 2-21-994 | N-Ph | O | t-bu | t-bu | b3 |
| 2-21-995 | N-Ph | O | methyl | methyl | b3 |
| 2-21-996 | N-Ph | O | b1 | b1 | b3 |
| 2-21-997 | N-Ph | O | b2 | b2 | b3 |
| 2-21-998 | N-Ph | O | b3 | b3 | b3 |
| 2-21-999 | N-Ph | O | H | H | b5 |
| 2-21-1000 | N-Ph | O | t-bu | t-bu | b5 |
| 2-21-1001 | N-Ph | O | methyl | methyl | b5 |
| 2-21-1002 | N-Ph | O | b1 | b1 | b5 |
| 2-21-1003 | N-Ph | O | b2 | b2 | b5 |
| 2-21-1004 | N-Ph | O | b3 | b3 | b5 |
| 2-21-1005 | N-Ph | O | H | H | b6 |
| 2-21-1006 | N-Ph | O | t-bu | t-bu | b6 |
| 2-21-1007 | N-Ph | O | methyl | methyl | b6 |
| 2-21-1008 | N-Ph | O | b1 | b1 | b6 |
| 2-21-1009 | N-Ph | O | b2 | b2 | b6 |
| 2-21-1010 | N-Ph | O | b3 | b3 | b6 |
| 2-21-1011 | N-Ph | O | H | H | b7 |
| 2-21-1012 | N-Ph | O | t-bu | t-bu | b7 |
| 2-21-1013 | N-Ph | O | methyl | methyl | b7 |
| 2-21-1014 | N-Ph | O | b1 | b1 | b7 |
| 2-21-1015 | N-Ph | O | b2 | b2 | b7 |
| 2-21-1016 | N-Ph | O | b3 | b3 | b7 |
| 2-21-1017 | N-Ph | S | H | H | H |
| 2-21-1018 | N-Ph | S | t-bu | t-bu | H |
| 2-21-1019 | N-Ph | S | methyl | methyl | H |
| 2-21-1020 | N-Ph | S | t-bu | t-bu | t-bu |
| 2-21-1021 | N-Ph | S | methyl | methyl | methyl |
| 2-21-1022 | N-Ph | S | b1 | b1 | H |
| 2-21-1023 | N-Ph | S | b2 | b2 | H |
| 2-21-1024 | N-Ph | S | b3 | b3 | H |
| 2-21-1025 | N-Ph | S | H | H | a1 |
| 2-21-1026 | N-Ph | S | t-bu | t-bu | a1 |
| 2-21-1027 | N-Ph | S | methyl | methyl | a1 |
| 2-21-1028 | N-Ph | S | b1 | b1 | a1 |
| 2-21-1029 | N-Ph | S | b2 | b2 | a1 |
| 2-21-1030 | N-Ph | S | b3 | b3 | a1 |
| 2-21-1031 | N-Ph | S | H | H | a2 |
| 2-21-1032 | N-Ph | S | t-bu | t-bu | a2 |
| 2-21-1033 | N-Ph | S | methyl | methyl | a2 |
| 2-21-1034 | N-Ph | S | b1 | b1 | a2 |
| 2-21-1035 | N-Ph | S | b2 | b2 | a2 |
| 2-21-1036 | N-Ph | S | b3 | b3 | a2 |
| 2-21-1037 | N-Ph | S | H | H | a3 |
| 2-21-1038 | N-Ph | S | t-bu | t-bu | a3 |
| 2-21-1039 | N-Ph | S | methyl | methyl | a3 |
| 2-21-1040 | N-Ph | S | b1 | b1 | a3 |
| 2-21-1041 | N-Ph | S | b2 | b2 | a3 |
| 2-21-1042 | N-Ph | S | b3 | b3 | a3 |
| 2-21-1043 | N-Ph | S | H | H | a4 |
| 2-21-1044 | N-Ph | S | t-bu | t-bu | a4 |
| 2-21-1045 | N-Ph | S | methyl | methyl | a4 |
| 2-21-1046 | N-Ph | S | b1 | b1 | a4 |
| 2-21-1047 | N-Ph | S | b2 | b2 | a4 |
| 2-21-1048 | N-Ph | S | b3 | b3 | a4 |
| 2-21-1049 | N-Ph | S | H | H | a5 |
| 2-21-1050 | N-Ph | S | t-bu | t-bu | a5 |
| 2-21-1051 | N-Ph | S | methyl | methyl | a5 |
| 2-21-1052 | N-Ph | S | b1 | b1 | a5 |
| 2-21-1053 | N-Ph | S | b2 | b2 | a5 |
| 2-21-1054 | N-Ph | S | b3 | b3 | a5 |
| 2-21-1055 | N-Ph | S | H | H | a6 |
| 2-21-1056 | N-Ph | S | t-bu | t-bu | a6 |
| 2-21-1057 | N-Ph | S | methyl | methyl | a6 |
| 2-21-1058 | N-Ph | S | b1 | b1 | a6 |
| 2-21-1059 | N-Ph | S | b2 | b2 | a6 |
| 2-21-1060 | N-Ph | S | b3 | b3 | a6 |
| 2-21-1061 | N-Ph | S | H | H | b1 |
| 2-21-1062 | N-Ph | S | t-bu | t-bu | b1 |
| 2-21-1063 | N-Ph | S | methyl | methyl | b1 |
| 2-21-1064 | N-Ph | S | b1 | b1 | b1 |
| 2-21-1065 | N-Ph | S | b2 | b2 | b1 |
| 2-21-1066 | N-Ph | S | b3 | b3 | b1 |
| 2-21-1067 | N-Ph | S | H | H | b2 |
| 2-21-1068 | N-Ph | S | t-bu | t-bu | b2 |
| 2-21-1069 | N-Ph | S | methyl | methyl | b2 |
| 2-21-1070 | N-Ph | S | b1 | b1 | b2 |
| 2-21-1071 | N-Ph | S | b2 | b2 | b2 |
| 2-21-1072 | N-Ph | S | b3 | b3 | b2 |
| 2-21-1073 | N-Ph | S | H | H | b3 |
| 2-21-1074 | N-Ph | S | t-bu | t-bu | b3 |
| 2-21-1075 | N-Ph | S | methyl | methyl | b3 |
| 2-21-1076 | N-Ph | S | b1 | b1 | b3 |
| 2-21-1077 | N-Ph | S | b2 | b2 | b3 |
| 2-21-1078 | N-Ph | S | b3 | b3 | b3 |
| 2-21-1079 | N-Ph | S | H | H | b4 |
| 2-21-1080 | N-Ph | S | t-bu | t-bu | b4 |
| 2-21-1081 | N-Ph | S | methyl | methyl | b4 |
| 2-21-1082 | N-Ph | S | b1 | b1 | b4 |
| 2-21-1083 | N-Ph | S | b2 | b2 | b4 |
| 2-21-1084 | N-Ph | S | b3 | b3 | b4 |
| 2-21-1085 | N-Ph | S | H | H | b5 |
| 2-21-1086 | N-Ph | S | t-bu | t-bu | b5 |
| 2-21-1087 | N-Ph | S | methyl | methyl | b5 |
| 2-21-1088 | N-Ph | S | b1 | b1 | b5 |
| 2-21-1089 | N-Ph | S | b2 | b2 | b5 |
| 2-21-1090 | N-Ph | S | b3 | b3 | b5 |
| 2-21-1091 | N-Ph | S | H | H | b6 |
| 2-21-1092 | N-Ph | S | t-bu | t-bu | b6 |
| 2-21-1093 | N-Ph | S | methyl | methyl | b6 |
| 2-21-1094 | N-Ph | S | b1 | b1 | b6 |
| 2-21-1095 | N-Ph | S | b2 | b2 | b6 |
| 2-21-1096 | N-Ph | S | b3 | b3 | b6 |
| 2-21-1097 | N-Ph | S | H | H | b7 |
| 2-21-1098 | N-Ph | S | t-bu | t-bu | b7 |
| 2-21-1099 | N-Ph | S | methyl | methyl | b7 |
| 2-21-1100 | N-Ph | S | b1 | b1 | b7 |
| 2-21-1101 | N-Ph | S | b2 | b2 | b7 |
| 2-21-1102 | N-Ph | S | b3 | b3 | b7 |
| 2-21-1103 | N-Ph | S | H | H | b8 |
| 2-21-1104 | N-Ph | S | t-bu | t-bu | b8 |
| 2-21-1105 | N-Ph | S | methyl | methyl | b8 |
| 2-21-1106 | N-Ph | S | b1 | b1 | b8 |
| 2-21-1107 | N-Ph | S | b2 | b2 | b8 |
| 2-21-1108 | N-Ph | S | b3 | b3 | b8 |
| 2-21-1109 | N-Ph | S | H | H | b9 |
| 2-21-1110 | N-Ph | S | t-bu | t-bu | b9 |
| 2-21-1111 | N-Ph | S | methyl | methyl | b9 |
| 2-21-1112 | N-Ph | S | b1 | b1 | b9 |
| 2-21-1113 | N-Ph | S | b2 | b2 | b9 |
| 2-21-1114 | N-Ph | S | b3 | b3 | b9 |
| 2-21-1115 | N-Ph | S | H | H | b10 |
| 2-21-1116 | N-Ph | S | t-bu | t-bu | b10 |
| 2-21-1117 | N-Ph | S | methyl | methyl | b10 |
| 2-21-1118 | N-Ph | S | b1 | b1 | b10 |
| 2-21-1119 | N-Ph | S | b2 | b2 | b10 |
| 2-21-1120 | N-Ph | S | b3 | b3 | b10 |
| 2-21-1121 | N-Ph | S | H | H | H |
| 2-21-1122 | N-Ph | S | t-bu | t-bu | H |
| 2-21-1123 | N-Ph | S | methyl | methyl | H |
| 2-21-1124 | N-Ph | S | b1 | b1 | H |
| 2-21-1125 | N-Ph | S | b2 | b2 | H |
| 2-21-1126 | N-Ph | S | b3 | b3 | H |
| 2-21-1127 | N-Ph | S | H | H | H |
| 2-21-1128 | N-Ph | S | t-bu | t-bu | H |
| 2-21-1129 | N-Ph | S | methyl | methyl | H |
| 2-21-1130 | N-Ph | S | b1 | b1 | H |
| 2-21-1131 | N-Ph | S | b2 | b2 | H |
| 2-21-1132 | N-Ph | S | b3 | b3 | H |
| 2-21-1133 | N-Ph | S | H | H | H |
| 2-21-1134 | N-Ph | S | t-bu | t-bu | H |
| 2-21-1135 | N-Ph | S | methyl | methyl | H |
| 2-21-1136 | N-Ph | S | b1 | b1 | H |
| 2-21-1137 | N-Ph | S | b2 | b2 | H |
| 2-21-1138 | N-Ph | S | b3 | b3 | H |
| 2-21-1139 | N-Ph | S | H | H | H |
| 2-21-1140 | N-Ph | S | t-bu | t-bu | H |
| 2-21-1141 | N-Ph | S | methyl | methyl | H |
| 2-21-1142 | N-Ph | S | b1 | b1 | H |
| 2-21-1143 | N-Ph | S | b2 | b2 | H |
| 2-21-1144 | N-Ph | S | b3 | b3 | H |
| 2-21-1145 | N-Ph | S | H | H | H |
| 2-21-1146 | N-Ph | S | t-bu | t-bu | H |
| 2-21-1147 | N-Ph | S | methyl | methyl | H |
| 2-21-1148 | N-Ph | S | b1 | b1 | H |
| 2-21-1149 | N-Ph | S | b2 | b2 | H |
| 2-21-1150 | N-Ph | S | b3 | b3 | H |
| 2-21-1151 | N-Ph | S | H | H | H |
| 2-21-1152 | N-Ph | S | t-bu | t-bu | H |
| 2-21-1153 | N-Ph | S | methyl | methyl | H |
| 2-21-1154 | N-Ph | S | b1 | b1 | H |
| 2-21-1155 | N-Ph | S | b2 | b2 | H |
| 2-21-1156 | N-Ph | S | b3 | b3 | H |
| 2-21-1157 | N-Ph | S | H | H | H |
| 2-21-1158 | N-Ph | S | t-bu | t-bu | H |
| 2-21-1159 | N-Ph | S | methyl | methyl | H |
| 2-21-1160 | N-Ph | S | b1 | b1 | H |
| 2-21-1161 | N-Ph | S | b2 | b2 | H |
| 2-21-1162 | N-Ph | S | b3 | b3 | H |
| 2-21-1163 | N-Ph | S | H | H | H |
| 2-21-1164 | N-Ph | S | t-bu | t-bu | H |
| 2-21-1165 | N-Ph | S | methyl | methyl | H |
| 2-21-1166 | N-Ph | S | b1 | b1 | H |
| 2-21-1167 | N-Ph | S | b2 | b2 | H |
| 2-21-1168 | N-Ph | S | b3 | b3 | H |
| 2-21-1169 | N-Ph | S | H | H | H |
| 2-21-1170 | N-Ph | S | t-bu | t-bu | H |
| 2-21-1171 | N-Ph | S | methyl | methyl | H |
| 2-21-1172 | N-Ph | S | b1 | b1 | H |
| 2-21-1173 | N-Ph | S | b2 | b2 | H |
| 2-21-1174 | N-Ph | S | b3 | b3 | H |
| 2-21-1175 | N-Ph | S | H | H | H |
| 2-21-1176 | N-Ph | S | t-bu | t-bu | H |
| 2-21-1177 | N-Ph | S | methyl | methyl | H |
| 2-21-1178 | N-Ph | S | b1 | b1 | H |
| 2-21-1179 | N-Ph | S | b2 | b2 | H |
| 2-21-1180 | N-Ph | S | b3 | b3 | H |
| 2-21-1181 | N-Ph | S | H | H | H |
| 2-21-1182 | N-Ph | S | t-bu | t-bu | H |
| 2-21-1183 | N-Ph | S | methyl | methyl | H |
| 2-21-1184 | N-Ph | S | b1 | b1 | H |
| 2-21-1185 | N-Ph | S | b2 | b2 | H |
| 2-21-1186 | N-Ph | S | b3 | b3 | H |
| 2-21-1187 | N-Ph | S | H | H | H |
| 2-21-1188 | N-Ph | S | t-bu | t-bu | H |
| 2-21-1189 | N-Ph | S | methyl | methyl | H |
| 2-21-1190 | N-Ph | S | b1 | b1 | H |
| 2-21-1191 | N-Ph | S | b2 | b2 | H |
| 2-21-1192 | N-Ph | S | b3 | b3 | H |
| 2-21-1193 | N-Ph | S | H | H | H |
| 2-21-1194 | N-Ph | S | t-bu | t-bu | H |
| 2-21-1195 | N-Ph | S | methyl | methyl | H |
| 2-21-1196 | N-Ph | S | b1 | b1 | H |
| 2-21-1197 | N-Ph | S | b2 | b2 | H |
| 2-21-1198 | N-Ph | S | b3 | b3 | H |
| 2-21-1199 | N-Ph | S | H | H | H |
| 2-21-1200 | N-Ph | S | t-bu | t-bu | H |
| 2-21-1201 | N-Ph | S | methyl | methyl | H |
| 2-21-1202 | N-Ph | S | b1 | b1 | H |
| 2-21-1203 | N-Ph | S | b2 | b2 | H |
| 2-21-1204 | N-Ph | S | b3 | b3 | H |
| 2-21-1205 | N-Ph | S | H | H | H |
| 2-21-1206 | N-Ph | S | t-bu | t-bu | H |
| 2-21-1207 | N-Ph | S | methyl | methyl | H |
| 2-21-1208 | N-Ph | S | b1 | b1 | H |
| 2-21-1209 | N-Ph | S | b2 | b2 | H |
| 2-21-1210 | N-Ph | S | b3 | b3 | H |
| 2-21-1211 | N-Ph | S | H | H | H |
| 2-21-1212 | N-Ph | S | t-bu | t-bu | H |
| 2-21-1213 | N-Ph | S | methyl | methyl | H |
| 2-21-1214 | N-Ph | S | b1 | b1 | H |
| 2-21-1215 | N-Ph | S | b2 | b2 | H |
| 2-21-1216 | N-Ph | S | b3 | b3 | H |
| 2-21-1217 | N-Ph | S | H | H | a1 |
| 2-21-1218 | N-Ph | S | t-bu | t-bu | a1 |
| 2-21-1219 | N-Ph | S | methyl | methyl | a1 |
| 2-21-1220 | N-Ph | S | b1 | b1 | a1 |
| 2-21-1221 | N-Ph | S | b2 | b2 | a1 |
| 2-21-1222 | N-Ph | S | b3 | b3 | a1 |
| 2-21-1223 | N-Ph | S | H | H | a2 |
| 2-21-1224 | N-Ph | S | t-bu | t-bu | a2 |
| 2-21-1225 | N-Ph | S | methyl | methyl | a2 |
| 2-21-1226 | N-Ph | S | b1 | b1 | a2 |
| 2-21-1227 | N-Ph | S | b2 | b2 | a2 |
| 2-21-1228 | N-Ph | S | b3 | b3 | a2 |
| 2-21-1229 | N-Ph | S | H | H | a3 |
| 2-21-1230 | N-Ph | S | t-bu | t-bu | a3 |
| 2-21-1231 | N-Ph | S | methyl | methyl | a3 |
| 2-21-1232 | N-Ph | S | b1 | b1 | a3 |
| 2-21-1233 | N-Ph | S | b2 | b2 | a3 |
| 2-21-1234 | N-Ph | S | b3 | b3 | a3 |
| 2-21-1235 | N-Ph | S | H | H | b1 |
| 2-21-1236 | N-Ph | S | t-bu | t-bu | b1 |
| 2-21-1237 | N-Ph | S | methyl | methyl | b1 |
| 2-21-1238 | N-Ph | S | b1 | b1 | b1 |
| 2-21-1239 | N-Ph | S | b2 | b2 | b1 |
| 2-21-1240 | N-Ph | S | b3 | b3 | b1 |
| 2-21-1241 | N-Ph | S | H | H | b2 |
| 2-21-1242 | N-Ph | S | t-bu | t-bu | b2 |
| 2-21-1243 | N-Ph | S | methyl | methyl | b2 |
| 2-21-1244 | N-Ph | S | b1 | b1 | b2 |
| 2-21-1245 | N-Ph | S | b2 | b2 | b2 |
| 2-21-1246 | N-Ph | S | b3 | b3 | b2 |
| 2-21-1247 | N-Ph | S | H | H | b3 |
| 2-21-1248 | N-Ph | S | t-bu | t-bu | b3 |
| 2-21-1249 | N-Ph | S | methyl | methyl | b3 |
| 2-21-1250 | N-Ph | S | b1 | b1 | b3 |
| 2-21-1251 | N-Ph | S | b2 | b2 | b3 |
| 2-21-1252 | N-Ph | S | b3 | b3 | b3 |
| 2-21-1253 | N-Ph | S | H | H | b5 |
| 2-21-1254 | N-Ph | S | t-bu | t-bu | b5 |
| 2-21-1255 | N-Ph | S | methyl | methyl | b5 |
| 2-21-1256 | N-Ph | S | b1 | b1 | b5 |
| 2-21-1257 | N-Ph | S | b2 | b2 | b5 |
| 2-21-1258 | N-Ph | S | b3 | b3 | b5 |
| 2-21-1259 | N-Ph | S | H | H | b6 |
| 2-21-1260 | N-Ph | S | t-bu | t-bu | b6 |
| 2-21-1261 | N-Ph | S | methyl | methyl | b6 |
| 2-21-1262 | N-Ph | S | b1 | b1 | b6 |
| 2-21-1263 | N-Ph | S | b2 | b2 | b6 |
| 2-21-1264 | N-Ph | S | b3 | b3 | b6 |
| 2-21-1265 | N-Ph | S | H | H | b7 |
| 2-21-1266 | N-Ph | S | t-bu | t-bu | b7 |
| 2-21-1267 | N-Ph | S | methyl | methyl | b7 |
| 2-21-1268 | N-Ph | S | b1 | b1 | b7 |
| 2-21-1269 | N-Ph | S | b2 | b2 | b7 |
| 2-21-1270 | N-Ph | S | b3 | b3 | b7 |

### Group 2-22

The compounds are represented by the formula 2-22 below, wherein CY₂ is a benzene group, X₁ is B, and Y1, Y2, Y3, R1b, R3a, (R2)a2 are each shown in the table 22 below,

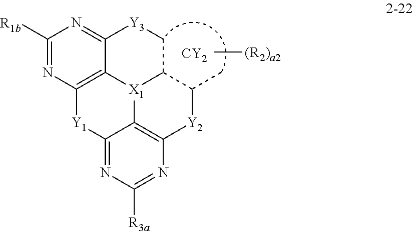

TABLE 22

| Compound | | | | | |
|---|---|---|---|---|---|
| No. | Y1 | Y2/Y3 | R1b | R3a | (R2)a2 |
| 2-22-001 | N-Ph | N-Ph | H | H | H |
| 2-22-002 | N-Ph | N-Ph | t-bu | t-bu | H |
| 2-22-003 | N-Ph | N-Ph | methyl | methyl | H |
| 2-22-004 | N-Ph | N-Ph | t-bu | t-bu | t-bu |
| 2-22-005 | N-Ph | N-Ph | methyl | methyl | methyl |
| 2-22-006 | N-Ph | N-Ph | b1 | b1 | H |
| 2-22-007 | N-Ph | N-Ph | b2 | b2 | H |
| 2-22-008 | N-Ph | N-Ph | b3 | b3 | H |
| 2-22-009 | N-Ph | N-Ph | H | H | a1 |
| 2-22-010 | N-Ph | N-Ph | t-bu | t-bu | a1 |
| 2-22-011 | N-Ph | N-Ph | methyl | methyl | a1 |
| 2-22-012 | N-Ph | N-Ph | b1 | b1 | a1 |
| 2-22-013 | N-Ph | N-Ph | b2 | b2 | a1 |
| 2-22-014 | N-Ph | N-Ph | b3 | b3 | a1 |
| 2-22-015 | N-Ph | N-Ph | H | H | a2 |
| 2-22-016 | N-Ph | N-Ph | t-bu | t-bu | a2 |
| 2-22-017 | N-Ph | N-Ph | methyl | methyl | a2 |
| 2-22-018 | N-Ph | N-Ph | b1 | b1 | a2 |
| 2-22-019 | N-Ph | N-Ph | b2 | b2 | a2 |
| 2-22-020 | N-Ph | N-Ph | b3 | b3 | a2 |
| 2-22-021 | N-Ph | N-Ph | H | H | a3 |
| 2-22-022 | N-Ph | N-Ph | t-bu | t-bu | a3 |
| 2-22-023 | N-Ph | N-Ph | methyl | methyl | a3 |
| 2-22-024 | N-Ph | N-Ph | b1 | b1 | a3 |
| 2-22-025 | N-Ph | N-Ph | b2 | b2 | a3 |
| 2-22-026 | N-Ph | N-Ph | b3 | b3 | a3 |
| 2-22-027 | N-Ph | N-Ph | H | H | a4 |
| 2-22-028 | N-Ph | N-Ph | t-bu | t-bu | a4 |
| 2-22-029 | N-Ph | N-Ph | methyl | methyl | a4 |
| 2-22-030 | N-Ph | N-Ph | b1 | b1 | a4 |
| 2-22-031 | N-Ph | N-Ph | b2 | b2 | a4 |
| 2-22-032 | N-Ph | N-Ph | b3 | b3 | a4 |
| 2-22-033 | N-Ph | N-Ph | H | H | a5 |
| 2-22-034 | N-Ph | N-Ph | t-bu | t-bu | a5 |
| 2-22-035 | N-Ph | N-Ph | methyl | methyl | a5 |
| 2-22-036 | N-Ph | N-Ph | b1 | b1 | a5 |
| 2-22-037 | N-Ph | N-Ph | b2 | b2 | a5 |
| 2-22-038 | N-Ph | N-Ph | b3 | b3 | a5 |
| 2-22-039 | N-Ph | N-Ph | H | H | a6 |
| 2-22-040 | N-Ph | N-Ph | t-bu | t-bu | a6 |
| 2-22-041 | N-Ph | N-Ph | methyl | methyl | a6 |
| 2-22-042 | N-Ph | N-Ph | b1 | b1 | a6 |
| 2-22-043 | N-Ph | N-Ph | b2 | b2 | a6 |
| 2-22-044 | N-Ph | N-Ph | b3 | b3 | a6 |
| 2-22-045 | N-Ph | N-Ph | H | H | b1 |
| 2-22-046 | N-Ph | N-Ph | t-bu | t-bu | b1 |
| 2-22-047 | N-Ph | N-Ph | methyl | methyl | b1 |
| 2-22-048 | N-Ph | N-Ph | b1 | b1 | b1 |
| 2-22-049 | N-Ph | N-Ph | b2 | b2 | b1 |
| 2-22-050 | N-Ph | N-Ph | b3 | b3 | b1 |
| 2-22-051 | N-Ph | N-Ph | H | H | b2 |
| 2-22-052 | N-Ph | N-Ph | t-bu | t-bu | b2 |
| 2-22-053 | N-Ph | N-Ph | methyl | methyl | b2 |
| 2-22-054 | N-Ph | N-Ph | b1 | b1 | b2 |
| 2-22-055 | N-Ph | N-Ph | b2 | b2 | b2 |
| 2-22-056 | N-Ph | N-Ph | b3 | b3 | b2 |
| 2-22-057 | N-Ph | N-Ph | H | H | b3 |
| 2-22-058 | N-Ph | N-Ph | t-bu | t-bu | b3 |
| 2-22-059 | N-Ph | N-Ph | methyl | methyl | b3 |
| 2-22-060 | N-Ph | N-Ph | b1 | b1 | b3 |
| 2-22-061 | N-Ph | N-Ph | b2 | b2 | b3 |
| 2-22-062 | N-Ph | N-Ph | b3 | b3 | b3 |
| 2-22-063 | N-Ph | N-Ph | H | H | b4 |
| 2-22-064 | N-Ph | N-Ph | t-bu | t-bu | b4 |
| 2-22-065 | N-Ph | N-Ph | methyl | methyl | b4 |
| 2-22-066 | N-Ph | N-Ph | b1 | b1 | b4 |
| 2-22-067 | N-Ph | N-Ph | b2 | b2 | b4 |
| 2-22-068 | N-Ph | N-Ph | b3 | b3 | b4 |
| 2-22-069 | N-Ph | N-Ph | H | H | b5 |
| 2-22-070 | N-Ph | N-Ph | t-bu | t-bu | b5 |
| 2-22-071 | N-Ph | N-Ph | methyl | methyl | b5 |
| 2-22-072 | N-Ph | N-Ph | b1 | b1 | b5 |
| 2-22-073 | N-Ph | N-Ph | b2 | b2 | b5 |
| 2-22-074 | N-Ph | N-Ph | b3 | b3 | b5 |
| 2-22-075 | N-Ph | N-Ph | H | H | b6 |
| 2-22-076 | N-Ph | N-Ph | t-bu | t-bu | b6 |
| 2-22-077 | N-Ph | N-Ph | methyl | methyl | b6 |
| 2-22-078 | N-Ph | N-Ph | b1 | b1 | b6 |
| 2-22-079 | N-Ph | N-Ph | b2 | b2 | b6 |
| 2-22-080 | N-Ph | N-Ph | b3 | b3 | b6 |
| 2-22-081 | N-Ph | N-Ph | H | H | b7 |
| 2-22-082 | N-Ph | N-Ph | t-bu | t-bu | b7 |
| 2-22-083 | N-Ph | N-Ph | methyl | methyl | b7 |
| 2-22-084 | N-Ph | N-Ph | b1 | b1 | b7 |
| 2-22-085 | N-Ph | N-Ph | b2 | b2 | b7 |
| 2-22-086 | N-Ph | N-Ph | b3 | b3 | b7 |
| 2-22-087 | N-Ph | N-Ph | H | H | b8 |
| 2-22-088 | N-Ph | N-Ph | t-bu | t-bu | b8 |
| 2-22-089 | N-Ph | N-Ph | methyl | methyl | b8 |
| 2-22-090 | N-Ph | N-Ph | b1 | b1 | b8 |
| 2-22-091 | N-Ph | N-Ph | b2 | b2 | b8 |
| 2-22-092 | N-Ph | N-Ph | b3 | b3 | b8 |
| 2-22-093 | N-Ph | N-Ph | H | H | b9 |
| 2-22-094 | N-Ph | N-Ph | t-bu | t-bu | b9 |
| 2-22-095 | N-Ph | N-Ph | methyl | methyl | b9 |
| 2-22-096 | N-Ph | N-Ph | b1 | b1 | b9 |
| 2-22-097 | N-Ph | N-Ph | b2 | b2 | b9 |
| 2-22-098 | N-Ph | N-Ph | b3 | b3 | b9 |
| 2-22-099 | N-Ph | N-Ph | H | H | b10 |
| 2-22-100 | N-Ph | N-Ph | t-bu | t-bu | b10 |
| 2-22-101 | N-Ph | N-Ph | methyl | methyl | b10 |
| 2-22-102 | N-Ph | N-Ph | b1 | b1 | b10 |
| 2-22-103 | N-Ph | N-Ph | b2 | b2 | b10 |
| 2-22-104 | N-Ph | N-Ph | b3 | b3 | b10 |
| 2-22-105 | N-Ph | N-Ph | H | H | H |
| 2-22-106 | N-Ph | N-Ph | t-bu | t-bu | H |
| 2-22-107 | N-Ph | N-Ph | methyl | methyl | H |
| 2-22-108 | N-Ph | N-Ph | b1 | b1 | H |
| 2-22-109 | N-Ph | N-Ph | b2 | b2 | H |
| 2-22-110 | N-Ph | N-Ph | b3 | b3 | H |
| 2-22-111 | N-Ph | N-Ph | H | H | H |
| 2-22-112 | N-Ph | N-Ph | t-bu | t-bu | H |
| 2-22-113 | N-Ph | N-Ph | methyl | methyl | H |
| 2-22-114 | N-Ph | N-Ph | b1 | b1 | H |
| 2-22-115 | N-Ph | N-Ph | b2 | b2 | H |
| 2-22-116 | N-Ph | N-Ph | b3 | b3 | H |
| 2-22-117 | N-Ph | N-Ph | H | H | H |
| 2-22-118 | N-Ph | N-Ph | t-bu | t-bu | H |
| 2-22-119 | N-Ph | N-Ph | methyl | methyl | H |
| 2-22-120 | N-Ph | N-Ph | b1 | b1 | H |
| 2-22-121 | N-Ph | N-Ph | b2 | b2 | H |
| 2-22-122 | N-Ph | N-Ph | b3 | b3 | H |
| 2-22-123 | N-Ph | N-Ph | H | H | H |
| 2-22-124 | N-Ph | N-Ph | t-bu | t-bu | H |
| 2-22-125 | N-Ph | N-Ph | methyl | methyl | H |
| 2-22-126 | N-Ph | N-Ph | b1 | b1 | H |
| 2-22-127 | N-Ph | N-Ph | b2 | b2 | H |
| 2-22-128 | N-Ph | N-Ph | b3 | b3 | H |
| 2-22-129 | N-Ph | N-Ph | H | H | H |
| 2-22-130 | N-Ph | N-Ph | t-bu | t-bu | H |
| 2-22-131 | N-Ph | N-Ph | methyl | methyl | H |
| 2-22-132 | N-Ph | N-Ph | b1 | b1 | H |
| 2-22-133 | N-Ph | N-Ph | b2 | b2 | H |
| 2-22-134 | N-Ph | N-Ph | b3 | b3 | H |
| 2-22-135 | N-Ph | N-Ph | H | H | H |
| 2-22-136 | N-Ph | N-Ph | t-bu | t-bu | H |
| 2-22-137 | N-Ph | N-Ph | methyl | methyl | H |
| 2-22-138 | N-Ph | N-Ph | b1 | b1 | H |
| 2-22-139 | N-Ph | N-Ph | b2 | b2 | H |
| 2-22-140 | N-Ph | N-Ph | b3 | b3 | H |
| 2-22-141 | N-Ph | N-Ph | H | H | H |
| 2-22-142 | N-Ph | N-Ph | t-bu | t-bu | H |
| 2-22-143 | N-Ph | N-Ph | methyl | methyl | H |
| 2-22-144 | N-Ph | N-Ph | b1 | b1 | H |
| 2-22-145 | N-Ph | N-Ph | b2 | b2 | H |
| 2-22-146 | N-Ph | N-Ph | b3 | b3 | H |
| 2-22-147 | N-Ph | N-Ph | H | H | H |
| 2-22-148 | N-Ph | N-Ph | t-bu | t-bu | H |
| 2-22-149 | N-Ph | N-Ph | methyl | methyl | H |
| 2-22-150 | N-Ph | N-Ph | b1 | b1 | H |
| 2-22-151 | N-Ph | N-Ph | b2 | b2 | H |
| 2-22-152 | N-Ph | N-Ph | b3 | b3 | H |
| 2-22-153 | N-Ph | N-Ph | H | H | H |
| 2-22-154 | N-Ph | N-Ph | t-bu | t-bu | H |
| 2-22-155 | N-Ph | N-Ph | methyl | methyl | H |
| 2-22-156 | N-Ph | N-Ph | b1 | b1 | H |
| 2-22-157 | N-Ph | N-Ph | b2 | b2 | H |
| 2-22-158 | N-Ph | N-Ph | b3 | b3 | H |
| 2-22-159 | N-Ph | N-Ph | H | H | H |
| 2-22-160 | N-Ph | N-Ph | t-bu | t-bu | H |
| 2-22-161 | N-Ph | N-Ph | methyl | methyl | H |
| 2-22-162 | N-Ph | N-Ph | b1 | b1 | H |
| 2-22-163 | N-Ph | N-Ph | b2 | b2 | H |
| 2-22-164 | N-Ph | N-Ph | b3 | b3 | H |
| 2-22-165 | N-Ph | N-Ph | H | H | H |
| 2-22-166 | N-Ph | N-Ph | t-bu | t-bu | H |
| 2-22-167 | N-Ph | N-Ph | methyl | methyl | H |
| 2-22-168 | N-Ph | N-Ph | b1 | b1 | H |
| 2-22-169 | N-Ph | N-Ph | b2 | b2 | H |
| 2-22-170 | N-Ph | N-Ph | b3 | b3 | H |
| 2-22-171 | N-Ph | N-Ph | H | H | H |
| 2-22-172 | N-Ph | N-Ph | t-bu | t-bu | H |
| 2-22-173 | N-Ph | N-Ph | methyl | methyl | H |
| 2-22-174 | N-Ph | N-Ph | b1 | b1 | H |
| 2-22-175 | N-Ph | N-Ph | b2 | b2 | H |
| 2-22-176 | N-Ph | N-Ph | b3 | b3 | H |
| 2-22-177 | N-Ph | N-Ph | H | H | H |
| 2-22-178 | N-Ph | N-Ph | t-bu | t-bu | H |
| 2-22-179 | N-Ph | N-Ph | methyl | methyl | H |
| 2-22-180 | N-Ph | N-Ph | b1 | b1 | H |
| 2-22-181 | N-Ph | N-Ph | b2 | b2 | H |
| 2-22-182 | N-Ph | N-Ph | b3 | b3 | H |
| 2-22-183 | N-Ph | N-Ph | H | H | H |
| 2-22-184 | N-Ph | N-Ph | t-bu | t-bu | H |
| 2-22-185 | N-Ph | N-Ph | methyl | methyl | H |
| 2-22-186 | N-Ph | N-Ph | b1 | b1 | H |
| 2-22-187 | N-Ph | N-Ph | b2 | b2 | H |
| 2-22-188 | N-Ph | N-Ph | b3 | b3 | H |
| 2-22-189 | N-Ph | N-Ph | H | H | H |
| 2-22-190 | N-Ph | N-Ph | t-bu | t-bu | H |
| 2-22-191 | N-Ph | N-Ph | methyl | methyl | H |
| 2-22-192 | N-Ph | N-Ph | b1 | b1 | H |
| 2-22-193 | N-Ph | N-Ph | b2 | b2 | H |
| 2-22-194 | N-Ph | N-Ph | b3 | b3 | H |
| 2-22-195 | N-Ph | N-Ph | H | H | H |
| 2-22-196 | N-Ph | N-Ph | t-bu | t-bu | H |
| 2-22-197 | N-Ph | N-Ph | methyl | methyl | H |
| 2-22-198 | N-Ph | N-Ph | b1 | b1 | H |
| 2-22-199 | N-Ph | N-Ph | b2 | b2 | H |
| 2-22-200 | N-Ph | N-Ph | b3 | b3 | H |
| 2-22-201 | N-Ph | N-Ph | H | H | a1 |
| 2-22-202 | N-Ph | N-Ph | t-bu | t-bu | a1 |
| 2-22-203 | N-Ph | N-Ph | methyl | methyl | a1 |
| 2-22-204 | N-Ph | N-Ph | b1 | b1 | a1 |
| 2-22-205 | N-Ph | N-Ph | b2 | b2 | a1 |
| 2-22-206 | N-Ph | N-Ph | b3 | b3 | a1 |
| 2-22-207 | N-Ph | N-Ph | H | H | a2 |
| 2-22-208 | N-Ph | N-Ph | t-bu | t-bu | a2 |
| 2-22-209 | N-Ph | N-Ph | methyl | methyl | a2 |
| 2-22-210 | N-Ph | N-Ph | b1 | b1 | a2 |
| 2-22-211 | N-Ph | N-Ph | b2 | b2 | a2 |
| 2-22-212 | N-Ph | N-Ph | b3 | b3 | a2 |
| 2-22-213 | N-Ph | N-Ph | H | H | a3 |
| 2-22-214 | N-Ph | N-Ph | t-bu | t-bu | a3 |
| 2-22-215 | N-Ph | N-Ph | methyl | methyl | a3 |
| 2-22-216 | N-Ph | N-Ph | b1 | b1 | a3 |
| 2-22-217 | N-Ph | N-Ph | b2 | b2 | a3 |
| 2-22-218 | N-Ph | N-Ph | b3 | b3 | a3 |
| 2-22-219 | N-Ph | N-Ph | H | H | b1 |
| 2-22-220 | N-Ph | N-Ph | t-bu | t-bu | b1 |
| 2-22-221 | N-Ph | N-Ph | methyl | methyl | b1 |
| 2-22-222 | N-Ph | N-Ph | b1 | b1 | b1 |
| 2-22-223 | N-Ph | N-Ph | b2 | b2 | b1 |
| 2-22-224 | N-Ph | N-Ph | b3 | b3 | b1 |
| 2-22-225 | N-Ph | N-Ph | H | H | b2 |
| 2-22-226 | N-Ph | N-Ph | t-bu | t-bu | b2 |
| 2-22-227 | N-Ph | N-Ph | methyl | methyl | b2 |
| 2-22-228 | N-Ph | N-Ph | b1 | b1 | b2 |
| 2-22-229 | N-Ph | N-Ph | b2 | b2 | b2 |
| 2-22-230 | N-Ph | N-Ph | b3 | b3 | b2 |
| 2-22-231 | N-Ph | N-Ph | H | H | b3 |
| 2-22-232 | N-Ph | N-Ph | t-bu | t-bu | b3 |
| 2-22-233 | N-Ph | N-Ph | methyl | methyl | b3 |
| 2-22-234 | N-Ph | N-Ph | b1 | b1 | b3 |
| 2-22-235 | N-Ph | N-Ph | b2 | b2 | b3 |
| 2-22-236 | N-Ph | N-Ph | b3 | b3 | b3 |
| 2-22-237 | N-Ph | N-Ph | H | H | b5 |
| 2-22-238 | N-Ph | N-Ph | t-bu | t-bu | b5 |
| 2-22-239 | N-Ph | N-Ph | methyl | methyl | b5 |
| 2-22-240 | N-Ph | N-Ph | b1 | b1 | b5 |
| 2-22-241 | N-Ph | N-Ph | b2 | b2 | b5 |
| 2-22-242 | N-Ph | N-Ph | b3 | b3 | b5 |
| 2-22-243 | N-Ph | N-Ph | H | H | b6 |
| 2-22-244 | N-Ph | N-Ph | t-bu | t-bu | b6 |
| 2-22-245 | N-Ph | N-Ph | methyl | methyl | b6 |
| 2-22-246 | N-Ph | N-Ph | b1 | b1 | b6 |
| 2-22-247 | N-Ph | N-Ph | b2 | b2 | b6 |
| 2-22-248 | N-Ph | N-Ph | b3 | b3 | b6 |
| 2-22-249 | N-Ph | N-Ph | H | H | b7 |
| 2-22-250 | N-Ph | N-Ph | t-bu | t-bu | b7 |
| 2-22-251 | N-Ph | N-Ph | methyl | methyl | b7 |
| 2-22-252 | N-Ph | N-Ph | b1 | b1 | b7 |
| 2-22-253 | N-Ph | N-Ph | b2 | b2 | b7 |
| 2-22-254 | N-Ph | N-Ph | b3 | b3 | b7 |
| 2-22-255 | N-Ph | N-Ph | H | H | H |
| 2-22-256 | N-tolyl | N-tolyl | t-bu | t-bu | H |
| 2-22-257 | N-tolyl | N-tolyl | methyl | methyl | H |
| 2-22-258 | N-tolyl | N-tolyl | t-bu | t-bu | t-bu |
| 2-22-259 | N-tolyl | N-tolyl | methyl | methyl | methyl |
| 2-22-260 | N-tolyl | N-tolyl | b1 | b1 | H |
| 2-22-261 | N-tolyl | N-tolyl | b2 | b2 | H |
| 2-22-262 | N-tolyl | N-tolyl | b3 | b3 | H |
| 2-22-263 | N-tolyl | N-tolyl | H | H | a1 |
| 2-22-264 | N-tolyl | N-tolyl | t-bu | t-bu | a1 |
| 2-22-265 | N-tolyl | N-tolyl | methyl | methyl | a1 |
| 2-22-266 | N-tolyl | N-tolyl | b1 | b1 | a1 |
| 2-22-267 | N-tolyl | N-tolyl | b2 | b2 | a1 |
| 2-22-268 | N-tolyl | N-tolyl | b3 | b3 | a1 |
| 2-22-269 | N-tolyl | N-tolyl | H | H | a2 |
| 2-22-270 | N-tolyl | N-tolyl | t-bu | t-bu | a2 |
| 2-22-271 | N-tolyl | N-tolyl | methyl | methyl | a2 |
| 2-22-272 | N-tolyl | N-tolyl | b1 | b1 | a2 |
| 2-22-273 | N-tolyl | N-tolyl | b2 | b2 | a2 |
| 2-22-274 | N-tolyl | N-tolyl | b3 | b3 | a2 |
| 2-22-275 | N-tolyl | N-tolyl | H | H | a3 |
| 2-22-276 | N-tolyl | N-tolyl | t-bu | t-bu | a3 |
| 2-22-277 | N-tolyl | N-tolyl | methyl | methyl | a3 |
| 2-22-278 | N-tolyl | N-tolyl | b1 | b1 | a3 |
| 2-22-279 | N-tolyl | N-tolyl | b2 | b2 | a3 |
| 2-22-280 | N-tolyl | N-tolyl | b3 | b3 | a3 |
| 2-22-281 | N-tolyl | N-tolyl | H | H | a4 |
| 2-22-282 | N-tolyl | N-tolyl | t-bu | t-bu | a4 |
| 2-22-283 | N-tolyl | N-tolyl | methyl | methyl | a4 |
| 2-22-284 | N-tolyl | N-tolyl | b1 | b1 | a4 |
| 2-22-285 | N-tolyl | N-tolyl | b2 | b2 | a4 |
| 2-22-286 | N-tolyl | N-tolyl | b3 | b3 | a4 |
| 2-22-287 | N-tolyl | N-tolyl | H | H | a5 |
| 2-22-288 | N-tolyl | N-tolyl | t-bu | t-bu | a5 |
| 2-22-289 | N-tolyl | N-tolyl | methyl | methyl | a5 |
| 2-22-290 | N-tolyl | N-tolyl | b1 | b1 | a5 |
| 2-22-291 | N-tolyl | N-tolyl | b2 | b2 | a5 |
| 2-22-292 | N-tolyl | N-tolyl | b3 | b3 | a5 |
| 2-22-293 | N-tolyl | N-tolyl | H | H | a6 |
| 2-22-294 | N-tolyl | N-tolyl | t-bu | t-bu | a6 |
| 2-22-295 | N-tolyl | N-tolyl | methyl | methyl | a6 |
| 2-22-296 | N-tolyl | N-tolyl | b1 | b1 | a6 |
| 2-22-297 | N-tolyl | N-tolyl | b2 | b2 | a6 |
| 2-22-298 | N-tolyl | N-tolyl | b3 | b3 | a6 |
| 2-22-299 | N-tolyl | N-tolyl | H | H | b1 |
| 2-22-300 | N-tolyl | N-tolyl | t-bu | t-bu | b1 |
| 2-22-301 | N-tolyl | N-tolyl | methyl | methyl | b1 |
| 2-22-302 | N-tolyl | N-tolyl | b1 | b1 | b1 |
| 2-22-303 | N-tolyl | N-tolyl | b2 | b2 | b1 |
| 2-22-304 | N-tolyl | N-tolyl | b3 | b3 | b1 |
| 2-22-305 | N-tolyl | N-tolyl | H | H | b2 |
| 2-22-306 | N-tolyl | N-tolyl | t-bu | t-bu | b2 |
| 2-22-307 | N-tolyl | N-tolyl | methyl | methyl | b2 |
| 2-22-308 | N-tolyl | N-tolyl | b1 | b1 | b2 |
| 2-22-309 | N-tolyl | N-tolyl | b2 | b2 | b2 |
| 2-22-310 | N-tolyl | N-tolyl | b3 | b3 | b2 |
| 2-22-311 | N-tolyl | N-tolyl | H | H | b3 |
| 2-22-312 | N-tolyl | N-tolyl | t-bu | t-bu | b3 |
| 2-22-313 | N-tolyl | N-tolyl | methyl | methyl | b3 |
| 2-22-314 | N-tolyl | N-tolyl | b1 | b1 | b3 |
| 2-22-315 | N-tolyl | N-tolyl | b2 | b2 | b3 |
| 2-22-316 | N-tolyl | N-tolyl | b3 | b3 | b3 |
| 2-22-317 | N-tolyl | N-tolyl | H | H | b4 |
| 2-22-318 | N-tolyl | N-tolyl | t-bu | t-bu | b4 |
| 2-22-319 | N-tolyl | N-tolyl | methyl | methyl | b4 |
| 2-22-320 | N-tolyl | N-tolyl | b1 | b1 | b4 |
| 2-22-321 | N-tolyl | N-tolyl | b2 | b2 | b4 |
| 2-22-322 | N-tolyl | N-tolyl | b3 | b3 | b4 |
| 2-22-323 | N-tolyl | N-tolyl | H | H | b5 |
| 2-22-324 | N-tolyl | N-tolyl | t-bu | t-bu | b5 |
| 2-22-325 | N-tolyl | N-tolyl | methyl | methyl | b5 |
| 2-22-326 | N-tolyl | N-tolyl | b1 | b1 | b5 |
| 2-22-327 | N-tolyl | N-tolyl | b2 | b2 | b5 |
| 2-22-328 | N-tolyl | N-tolyl | b3 | b3 | b5 |
| 2-22-329 | N-tolyl | N-tolyl | H | H | b6 |
| 2-22-330 | N-tolyl | N-tolyl | t-bu | t-bu | b6 |
| 2-22-331 | N-tolyl | N-tolyl | methyl | methyl | b6 |
| 2-22-332 | N-tolyl | N-tolyl | b1 | b1 | b6 |
| 2-22-333 | N-tolyl | N-tolyl | b2 | b2 | b6 |
| 2-22-334 | N-tolyl | N-tolyl | b3 | b3 | b6 |
| 2-22-335 | N-tolyl | N-tolyl | H | H | b7 |
| 2-22-336 | N-tolyl | N-tolyl | t-bu | t-bu | b7 |
| 2-22-337 | N-tolyl | N-tolyl | methyl | methyl | b7 |
| 2-22-338 | N-tolyl | N-tolyl | b1 | b1 | b7 |
| 2-22-339 | N-tolyl | N-tolyl | b2 | b2 | b7 |
| 2-22-340 | N-tolyl | N-tolyl | b3 | b3 | b7 |
| 2-22-341 | N-tolyl | N-tolyl | H | H | b8 |
| 2-22-342 | N-tolyl | N-tolyl | t-bu | t-bu | b8 |
| 2-22-343 | N-tolyl | N-tolyl | methyl | methyl | b8 |
| 2-22-344 | N-tolyl | N-tolyl | b1 | b1 | b8 |
| 2-22-345 | N-tolyl | N-tolyl | b2 | b2 | b8 |
| 2-22-346 | N-tolyl | N-tolyl | b3 | b3 | b8 |
| 2-22-347 | N-tolyl | N-tolyl | H | H | b9 |
| 2-22-348 | N-tolyl | N-tolyl | t-bu | t-bu | b9 |
| 2-22-349 | N-tolyl | N-tolyl | methyl | methyl | b9 |
| 2-22-350 | N-tolyl | N-tolyl | b1 | b1 | b9 |
| 2-22-351 | N-tolyl | N-tolyl | b2 | b2 | b9 |
| 2-22-352 | N-tolyl | N-tolyl | b3 | b3 | b9 |
| 2-22-353 | N-tolyl | N-tolyl | H | H | b10 |
| 2-22-354 | N-tolyl | N-tolyl | t-bu | t-bu | b10 |
| 2-22-355 | N-tolyl | N-tolyl | methyl | methyl | b10 |
| 2-22-356 | N-tolyl | N-tolyl | b1 | b1 | b10 |
| 2-22-357 | N-tolyl | N-tolyl | b2 | b2 | b10 |
| 2-22-358 | N-tolyl | N-tolyl | b3 | b3 | b10 |
| 2-22-359 | N-tolyl | N-tolyl | H | H | H |
| 2-22-360 | N-tolyl | N-tolyl | t-bu | t-bu | H |
| 2-22-361 | N-tolyl | N-tolyl | methyl | methyl | H |
| 2-22-362 | N-tolyl | N-tolyl | b1 | b1 | H |
| 2-22-363 | N-tolyl | N-tolyl | b2 | b2 | H |
| 2-22-364 | N-tolyl | N-tolyl | b3 | b3 | H |
| 2-22-365 | N-tolyl | N-tolyl | H | H | H |
| 2-22-366 | N-tolyl | N-tolyl | t-bu | t-bu | H |
| 2-22-367 | N-tolyl | N-tolyl | methyl | methyl | H |
| 2-22-368 | N-tolyl | N-tolyl | b1 | b1 | H |
| 2-22-369 | N-tolyl | N-tolyl | b2 | b2 | H |
| 2-22-370 | N-tolyl | N-tolyl | b3 | b3 | H |
| 2-22-371 | N-tolyl | N-tolyl | H | H | H |
| 2-22-372 | N-tolyl | N-tolyl | t-bu | t-bu | H |
| 2-22-373 | N-tolyl | N-tolyl | methyl | methyl | H |
| 2-22-374 | N-tolyl | N-tolyl | b1 | b1 | H |
| 2-22-375 | N-tolyl | N-tolyl | b2 | b2 | H |
| 2-22-376 | N-tolyl | N-tolyl | b3 | b3 | H |
| 2-22-377 | N-tolyl | N-tolyl | H | H | H |
| 2-22-378 | N-tolyl | N-tolyl | t-bu | t-bu | H |
| 2-22-379 | N-tolyl | N-tolyl | methyl | methyl | H |
| 2-22-380 | N-tolyl | N-tolyl | b1 | b1 | H |
| 2-22-381 | N-tolyl | N-tolyl | b2 | b2 | H |
| 2-22-382 | N-tolyl | N-tolyl | b3 | b3 | H |
| 2-22-383 | N-tolyl | N-tolyl | H | H | H |
| 2-22-384 | N-tolyl | N-tolyl | t-bu | t-bu | H |
| 2-22-385 | N-tolyl | N-tolyl | methyl | methyl | H |
| 2-22-386 | N-tolyl | N-tolyl | b1 | b1 | H |
| 2-22-387 | N-tolyl | N-tolyl | b2 | b2 | H |
| 2-22-388 | N-tolyl | N-tolyl | b3 | b3 | H |
| 2-22-389 | N-tolyl | N-tolyl | H | H | H |
| 2-22-390 | N-tolyl | N-tolyl | t-bu | t-bu | H |
| 2-22-391 | N-tolyl | N-tolyl | methyl | methyl | H |
| 2-22-392 | N-tolyl | N-tolyl | b1 | b1 | H |
| 2-22-393 | N-tolyl | N-tolyl | b2 | b2 | H |
| 2-22-394 | N-tolyl | N-tolyl | b3 | b3 | H |
| 2-22-395 | N-tolyl | N-tolyl | H | H | H |
| 2-22-396 | N-tolyl | N-tolyl | t-bu | t-bu | H |
| 2-22-397 | N-tolyl | N-tolyl | methyl | methyl | H |
| 2-22-398 | N-tolyl | N-tolyl | b1 | b1 | H |
| 2-22-399 | N-tolyl | N-tolyl | b2 | b2 | H |
| 2-22-400 | N-tolyl | N-tolyl | b3 | b3 | H |
| 2-22-401 | N-tolyl | N-tolyl | H | H | H |
| 2-22-402 | N-tolyl | N-tolyl | t-bu | t-bu | H |
| 2-22-403 | N-tolyl | N-tolyl | methyl | methyl | H |
| 2-22-404 | N-tolyl | N-tolyl | b1 | b1 | H |
| 2-22-405 | N-tolyl | N-tolyl | b2 | b2 | H |
| 2-22-406 | N-tolyl | N-tolyl | b3 | b3 | H |
| 2-22-407 | N-tolyl | N-tolyl | H | H | H |
| 2-22-408 | N-tolyl | N-tolyl | t-bu | t-bu | H |
| 2-22-409 | N-tolyl | N-tolyl | methyl | methyl | H |
| 2-22-410 | N-tolyl | N-tolyl | b1 | b1 | H |
| 2-22-411 | N-tolyl | N-tolyl | b2 | b2 | H |
| 2-22-412 | N-tolyl | N-tolyl | b3 | b3 | H |
| 2-22-413 | N-tolyl | N-tolyl | H | H | H |
| 2-22-414 | N-tolyl | N-tolyl | t-bu | t-bu | H |
| 2-22-415 | N-tolyl | N-tolyl | methyl | methyl | H |
| 2-22-416 | N-tolyl | N-tolyl | b1 | b1 | H |
| 2-22-417 | N-tolyl | N-tolyl | b2 | b2 | H |
| 2-22-418 | N-tolyl | N-tolyl | b3 | b3 | H |
| 2-22-419 | N-tolyl | N-tolyl | H | H | H |
| 2-22-420 | N-tolyl | N-tolyl | t-bu | t-bu | H |
| 2-22-421 | N-tolyl | N-tolyl | methyl | methyl | H |
| 2-22-422 | N-tolyl | N-tolyl | b1 | b1 | H |
| 2-22-423 | N-tolyl | N-tolyl | b2 | b2 | H |
| 2-22-424 | N-tolyl | N-tolyl | b3 | b3 | H |
| 2-22-425 | N-tolyl | N-tolyl | H | H | H |
| 2-22-426 | N-tolyl | N-tolyl | t-bu | t-bu | H |
| 2-22-427 | N-tolyl | N-tolyl | methyl | methyl | H |
| 2-22-428 | N-tolyl | N-tolyl | b1 | b1 | H |
| 2-22-429 | N-tolyl | N-tolyl | b2 | b2 | H |
| 2-22-430 | N-tolyl | N-tolyl | b3 | b3 | H |
| 2-22-431 | N-tolyl | N-tolyl | H | H | H |
| 2-22-432 | N-tolyl | N-tolyl | t-bu | t-bu | H |
| 2-22-433 | N-tolyl | N-tolyl | methyl | methyl | H |
| 2-22-434 | N-tolyl | N-tolyl | b1 | b1 | H |
| 2-22-435 | N-tolyl | N-tolyl | b2 | b2 | H |
| 2-22-436 | N-tolyl | N-tolyl | b3 | b3 | H |
| 2-22-437 | N-tolyl | N-tolyl | H | H | H |
| 2-22-438 | N-tolyl | N-tolyl | t-bu | t-bu | H |
| 2-22-439 | N-tolyl | N-tolyl | methyl | methyl | H |
| 2-22-440 | N-tolyl | N-tolyl | b1 | b1 | H |
| 2-22-441 | N-tolyl | N-tolyl | b2 | b2 | H |
| 2-22-442 | N-tolyl | N-tolyl | b3 | b3 | H |
| 2-22-443 | N-tolyl | N-tolyl | H | H | H |
| 2-22-444 | N-tolyl | N-tolyl | t-bu | t-bu | H |
| 2-22-445 | N-tolyl | N-tolyl | methyl | methyl | H |
| 2-22-446 | N-tolyl | N-tolyl | b1 | b1 | H |
| 2-22-447 | N-tolyl | N-tolyl | b2 | b2 | H |
| 2-22-448 | N-tolyl | N-tolyl | b3 | b3 | H |
| 2-22-449 | N-tolyl | N-tolyl | H | H | H |
| 2-22-450 | N-tolyl | N-tolyl | t-bu | t-bu | H |
| 2-22-451 | N-tolyl | N-tolyl | methyl | methyl | H |
| 2-22-452 | N-tolyl | N-tolyl | b1 | b1 | H |
| 2-22-453 | N-tolyl | N-tolyl | b2 | b2 | H |
| 2-22-454 | N-tolyl | N-tolyl | b3 | b3 | H |
| 2-22-455 | N-tolyl | N-tolyl | H | H | a1 |
| 2-22-456 | N-tolyl | N-tolyl | t-bu | t-bu | a1 |
| 2-22-457 | N-tolyl | N-tolyl | methyl | methyl | a1 |
| 2-22-458 | N-tolyl | N-tolyl | b1 | b1 | a1 |
| 2-22-459 | N-tolyl | N-tolyl | b2 | b2 | a1 |
| 2-22-460 | N-tolyl | N-tolyl | b3 | b3 | a1 |
| 2-22-461 | N-tolyl | N-tolyl | H | H | a2 |
| 2-22-462 | N-tolyl | N-tolyl | t-bu | t-bu | a2 |
| 2-22-463 | N-tolyl | N-tolyl | methyl | methyl | a2 |
| 2-22-464 | N-tolyl | N-tolyl | b1 | b1 | a2 |
| 2-22-465 | N-tolyl | N-tolyl | b2 | b2 | a2 |
| 2-22-466 | N-tolyl | N-tolyl | b3 | b3 | a2 |
| 2-22-467 | N-tolyl | N-tolyl | H | H | a3 |
| 2-22-468 | N-tolyl | N-tolyl | t-bu | t-bu | a3 |
| 2-22-469 | N-tolyl | N-tolyl | methyl | methyl | a3 |
| 2-22-470 | N-tolyl | N-tolyl | b1 | b1 | a3 |
| 2-22-471 | N-tolyl | N-tolyl | b2 | b2 | a3 |
| 2-22-472 | N-tolyl | N-tolyl | b3 | b3 | a3 |
| 2-22-473 | N-tolyl | N-tolyl | H | H | b1 |
| 2-22-474 | N-tolyl | N-tolyl | t-bu | t-bu | b1 |
| 2-22-475 | N-tolyl | N-tolyl | methyl | methyl | b1 |
| 2-22-476 | N-tolyl | N-tolyl | b1 | b1 | b1 |
| 2-22-477 | N-tolyl | N-tolyl | b2 | b2 | b1 |
| 2-22-478 | N-tolyl | N-tolyl | b3 | b3 | b1 |
| 2-22-479 | N-tolyl | N-tolyl | H | H | b2 |
| 2-22-480 | N-tolyl | N-tolyl | t-bu | t-bu | b2 |
| 2-22-481 | N-tolyl | N-tolyl | methyl | methyl | b2 |
| 2-22-482 | N-tolyl | N-tolyl | b1 | b1 | b2 |
| 2-22-483 | N-tolyl | N-tolyl | b2 | b2 | b2 |
| 2-22-484 | N-tolyl | N-tolyl | b3 | b3 | b2 |
| 2-22-485 | N-tolyl | N-tolyl | H | H | b3 |
| 2-22-486 | N-tolyl | N-tolyl | t-bu | t-bu | b3 |
| 2-22-487 | N-tolyl | N-tolyl | methyl | methyl | b3 |
| 2-22-488 | N-tolyl | N-tolyl | b1 | b1 | b3 |
| 2-22-489 | N-tolyl | N-tolyl | b2 | b2 | b3 |
| 2-22-490 | N-tolyl | N-tolyl | b3 | b3 | b3 |
| 2-22-491 | N-tolyl | N-tolyl | H | H | b5 |
| 2-22-492 | N-tolyl | N-tolyl | t-bu | t-bu | b5 |
| 2-22-493 | N-tolyl | N-tolyl | methyl | methyl | b5 |
| 2-22-494 | N-tolyl | N-tolyl | b1 | b1 | b5 |
| 2-22-495 | N-tolyl | N-tolyl | b2 | b2 | b5 |
| 2-22-496 | N-tolyl | N-tolyl | b3 | b3 | b5 |
| 2-22-497 | N-tolyl | N-tolyl | H | H | b6 |
| 2-22-498 | N-tolyl | N-tolyl | t-bu | t-bu | b6 |
| 2-22-499 | N-tolyl | N-tolyl | methyl | methyl | b6 |
| 2-22-500 | N-tolyl | N-tolyl | b1 | b1 | b6 |
| 2-22-501 | N-tolyl | N-tolyl | b2 | b2 | b6 |
| 2-22-502 | N-tolyl | N-tolyl | b3 | b3 | b6 |
| 2-22-503 | N-tolyl | N-tolyl | H | H | b7 |
| 2-22-504 | N-tolyl | N-tolyl | t-bu | t-bu | b7 |
| 2-22-505 | N-tolyl | N-tolyl | methyl | methyl | b7 |
| 2-22-506 | N-tolyl | N-tolyl | b1 | b1 | b7 |
| 2-22-507 | N-tolyl | N-tolyl | b2 | b2 | b7 |
| 2-22-508 | N-tolyl | N-tolyl | b3 | b3 | b7 |
| 2-22-509 | N-Biph | N-Biph | H | H | H |
| 2-22-510 | N-Biph | N-Biph | t-bu | t-bu | H |
| 2-22-511 | N-Biph | N-Biph | methyl | methyl | H |
| 2-22-512 | N-Biph | N-Biph | t-bu | t-bu | t-bu |
| 2-22-513 | N-Biph | N-Biph | methyl | methyl | methyl |
| 2-22-514 | N-Biph | N-Biph | b1 | b1 | H |
| 2-22-515 | N-Biph | N-Biph | b2 | b2 | H |
| 2-22-516 | N-Biph | N-Biph | b3 | b3 | H |
| 2-22-517 | N-Biph | N-Biph | H | H | a1 |
| 2-22-518 | N-Biph | N-Biph | t-bu | t-bu | a1 |
| 2-22-519 | N-Biph | N-Biph | methyl | methyl | a1 |
| 2-22-520 | N-Biph | N-Biph | b1 | b1 | a1 |
| 2-22-521 | N-Biph | N-Biph | b2 | b2 | a1 |
| 2-22-522 | N-Biph | N-Biph | b3 | b3 | a1 |
| 2-22-523 | N-Biph | N-Biph | H | H | a2 |
| 2-22-524 | N-Biph | N-Biph | t-bu | t-bu | a2 |
| 2-22-525 | N-Biph | N-Biph | methyl | methyl | a2 |
| 2-22-526 | N-Biph | N-Biph | b1 | b1 | a2 |
| 2-22-527 | N-Biph | N-Biph | b2 | b2 | a2 |
| 2-22-528 | N-Biph | N-Biph | b3 | b3 | a2 |
| 2-22-529 | N-Biph | N-Biph | H | H | a3 |
| 2-22-530 | N-Biph | N-Biph | t-bu | t-bu | a3 |
| 2-22-531 | N-Biph | N-Biph | methyl | methyl | a3 |
| 2-22-532 | N-Biph | N-Biph | b1 | b1 | a3 |
| 2-22-533 | N-Biph | N-Biph | b2 | b2 | a3 |
| 2-22-534 | N-Biph | N-Biph | b3 | b3 | a3 |
| 2-22-535 | N-Biph | N-Biph | H | H | a4 |
| 2-22-536 | N-Biph | N-Biph | t-bu | t-bu | a4 |
| 2-22-537 | N-Biph | N-Biph | methyl | methyl | a4 |
| 2-22-538 | N-Biph | N-Biph | b1 | b1 | a4 |
| 2-22-539 | N-Biph | N-Biph | b2 | b2 | a4 |
| 2-22-540 | N-Biph | N-Biph | b3 | b3 | a4 |
| 2-22-541 | N-Biph | N-Biph | H | H | a5 |
| 2-22-542 | N-Biph | N-Biph | t-bu | t-bu | a5 |
| 2-22-543 | N-Biph | N-Biph | methyl | methyl | a5 |
| 2-22-544 | N-Biph | N-Biph | b1 | b1 | a5 |
| 2-22-545 | N-Biph | N-Biph | b2 | b2 | a5 |
| 2-22-546 | N-Biph | N-Biph | b3 | b3 | a5 |
| 2-22-547 | N-Biph | N-Biph | H | H | a6 |
| 2-22-548 | N-Biph | N-Biph | t-bu | t-bu | a6 |
| 2-22-549 | N-Biph | N-Biph | methyl | methyl | a6 |
| 2-22-550 | N-Biph | N-Biph | b1 | b1 | a6 |
| 2-22-551 | N-Biph | N-Biph | b2 | b2 | a6 |
| 2-22-552 | N-Biph | N-Biph | b3 | b3 | a6 |
| 2-22-553 | N-Biph | N-Biph | H | H | b1 |
| 2-22-554 | N-Biph | N-Biph | t-bu | t-bu | b1 |
| 2-22-555 | N-Biph | N-Biph | methyl | methyl | b1 |
| 2-22-556 | N-Biph | N-Biph | b1 | b1 | b1 |
| 2-22-557 | N-Biph | N-Biph | b2 | b2 | b1 |
| 2-22-558 | N-Biph | N-Biph | b3 | b3 | b1 |
| 2-22-559 | N-Biph | N-Biph | H | H | b2 |
| 2-22-560 | N-Biph | N-Biph | t-bu | t-bu | b2 |
| 2-22-561 | N-Biph | N-Biph | methyl | methyl | b2 |
| 2-22-562 | N-Biph | N-Biph | b1 | b1 | b2 |
| 2-22-563 | N-Biph | N-Biph | b2 | b2 | b2 |
| 2-22-564 | N-Biph | N-Biph | b3 | b3 | b2 |
| 2-22-565 | N-Biph | N-Biph | H | H | b3 |
| 2-22-566 | N-Biph | N-Biph | t-bu | t-bu | b3 |
| 2-22-567 | N-Biph | N-Biph | methyl | methyl | b3 |
| 2-22-568 | N-Biph | N-Biph | b1 | b1 | b3 |
| 2-22-569 | N-Biph | N-Biph | b2 | b2 | b3 |
| 2-22-570 | N-Biph | N-Biph | b3 | b3 | b3 |
| 2-22-571 | N-Biph | N-Biph | H | H | b4 |
| 2-22-572 | N-Biph | N-Biph | t-bu | t-bu | b4 |
| 2-22-573 | N-Biph | N-Biph | methyl | methyl | b4 |
| 2-22-574 | N-Biph | N-Biph | b1 | b1 | b4 |
| 2-22-575 | N-Biph | N-Biph | b2 | b2 | b4 |
| 2-22-576 | N-Biph | N-Biph | b3 | b3 | b4 |
| 2-22-577 | N-Biph | N-Biph | H | H | b5 |
| 2-22-578 | N-Biph | N-Biph | t-bu | t-bu | b5 |
| 2-22-579 | N-Biph | N-Biph | methyl | methyl | b5 |
| 2-22-580 | N-Biph | N-Biph | b1 | b1 | b5 |
| 2-22-581 | N-Biph | N-Biph | b2 | b2 | b5 |
| 2-22-582 | N-Biph | N-Biph | b3 | b3 | b5 |
| 2-22-583 | N-Biph | N-Biph | H | H | b6 |
| 2-22-584 | N-Biph | N-Biph | t-bu | t-bu | b6 |
| 2-22-585 | N-Biph | N-Biph | methyl | methyl | b6 |
| 2-22-586 | N-Biph | N-Biph | b1 | b1 | b6 |
| 2-22-587 | N-Biph | N-Biph | b2 | b2 | b6 |
| 2-22-588 | N-Biph | N-Biph | b3 | b3 | b6 |
| 2-22-589 | N-Biph | N-Biph | H | H | b7 |
| 2-22-590 | N-Biph | N-Biph | t-bu | t-bu | b7 |
| 2-22-591 | N-Biph | N-Biph | methyl | methyl | b7 |
| 2-22-592 | N-Biph | N-Biph | b1 | b1 | b7 |
| 2-22-593 | N-Biph | N-Biph | b2 | b2 | b7 |
| 2-22-594 | N-Biph | N-Biph | b3 | b3 | b7 |
| 2-22-595 | N-Biph | N-Biph | H | H | b8 |
| 2-22-596 | N-Biph | N-Biph | t-bu | t-bu | b8 |
| 2-22-597 | N-Biph | N-Biph | methyl | methyl | b8 |
| 2-22-598 | N-Biph | N-Biph | b1 | b1 | b8 |
| 2-22-599 | N-Biph | N-Biph | b2 | b2 | b8 |
| 2-22-600 | N-Biph | N-Biph | b3 | b3 | b8 |
| 2-22-601 | N-Biph | N-Biph | H | H | b9 |
| 2-22-602 | N-Biph | N-Biph | t-bu | t-bu | b9 |
| 2-22-603 | N-Biph | N-Biph | methyl | methyl | b9 |
| 2-22-604 | N-Biph | N-Biph | b1 | b1 | b9 |
| 2-22-605 | N-Biph | N-Biph | b2 | b2 | b9 |
| 2-22-606 | N-Biph | N-Biph | b3 | b3 | b9 |
| 2-22-607 | N-Biph | N-Biph | H | H | b10 |
| 2-22-608 | N-Biph | N-Biph | t-bu | t-bu | b10 |
| 2-22-609 | N-Biph | N-Biph | methyl | methyl | b10 |
| 2-22-610 | N-Biph | N-Biph | b1 | b1 | b10 |
| 2-22-611 | N-Biph | N-Biph | b2 | b2 | b10 |
| 2-22-612 | N-Biph | N-Biph | b3 | b3 | b10 |
| 2-22-613 | N-Biph | N-Biph | H | H | H |
| 2-22-614 | N-Biph | N-Biph | t-bu | t-bu | H |
| 2-22-615 | N-Biph | N-Biph | methyl | methyl | H |
| 2-22-616 | N-Biph | N-Biph | b1 | b1 | H |
| 2-22-617 | N-Biph | N-Biph | b2 | b2 | H |
| 2-22-618 | N-Biph | N-Biph | b3 | b3 | H |
| 2-22-619 | N-Biph | N-Biph | H | H | H |
| 2-22-620 | N-Biph | N-Biph | t-bu | t-bu | H |
| 2-22-621 | N-Biph | N-Biph | methyl | methyl | H |
| 2-22-622 | N-Biph | N-Biph | b1 | b1 | H |
| 2-22-623 | N-Biph | N-Biph | b2 | b2 | H |
| 2-22-624 | N-Biph | N-Biph | b3 | b3 | H |
| 2-22-625 | N-Biph | N-Biph | H | H | H |
| 2-22-626 | N-Biph | N-Biph | t-bu | t-bu | H |
| 2-22-627 | N-Biph | N-Biph | methyl | methyl | H |
| 2-22-628 | N-Biph | N-Biph | b1 | b1 | H |
| 2-22-629 | N-Biph | N-Biph | b2 | b2 | H |
| 2-22-630 | N-Biph | N-Biph | b3 | b3 | H |
| 2-22-631 | N-Biph | N-Biph | H | H | H |
| 2-22-632 | N-Biph | N-Biph | t-bu | t-bu | H |
| 2-22-633 | N-Biph | N-Biph | methyl | methyl | H |
| 2-22-634 | N-Biph | N-Biph | b1 | b1 | H |
| 2-22-635 | N-Biph | N-Biph | b2 | b2 | H |
| 2-22-636 | N-Biph | N-Biph | b3 | b3 | H |
| 2-22-637 | N-Biph | N-Biph | H | H | H |
| 2-22-638 | N-Biph | N-Biph | t-bu | t-bu | H |
| 2-22-639 | N-Biph | N-Biph | methyl | methyl | H |
| 2-22-640 | N-Biph | N-Biph | b1 | b1 | H |
| 2-22-641 | N-Biph | N-Biph | b2 | b2 | H |
| 2-22-642 | N-Biph | N-Biph | b3 | b3 | H |
| 2-22-643 | N-Biph | N-Biph | H | H | H |
| 2-22-644 | N-Biph | N-Biph | t-bu | t-bu | H |
| 2-22-645 | N-Biph | N-Biph | methyl | methyl | H |
| 2-22-646 | N-Biph | N-Biph | b1 | b1 | H |
| 2-22-647 | N-Biph | N-Biph | b2 | b2 | H |
| 2-22-648 | N-Biph | N-Biph | b3 | b3 | H |
| 2-22-649 | N-Biph | N-Biph | H | H | H |
| 2-22-650 | N-Biph | N-Biph | t-bu | t-bu | H |
| 2-22-651 | N-Biph | N-Biph | methyl | methyl | H |
| 2-22-652 | N-Biph | N-Biph | b1 | b1 | H |
| 2-22-653 | N-Biph | N-Biph | b2 | b2 | H |
| 2-22-654 | N-Biph | N-Biph | b3 | b3 | H |
| 2-22-655 | N-Biph | N-Biph | H | H | H |
| 2-22-656 | N-Biph | N-Biph | t-bu | t-bu | H |
| 2-22-657 | N-Biph | N-Biph | methyl | methyl | H |
| 2-22-658 | N-Biph | N-Biph | b1 | b1 | H |
| 2-22-659 | N-Biph | N-Biph | b2 | b2 | H |
| 2-22-660 | N-Biph | N-Biph | b3 | b3 | H |
| 2-22-661 | N-Biph | N-Biph | H | H | H |
| 2-22-662 | N-Biph | N-Biph | t-bu | t-bu | H |
| 2-22-663 | N-Biph | N-Biph | methyl | methyl | H |
| 2-22-664 | N-Biph | N-Biph | b1 | b1 | H |
| 2-22-665 | N-Biph | N-Biph | b2 | b2 | H |
| 2-22-666 | N-Biph | N-Biph | b3 | b3 | H |
| 2-22-667 | N-Biph | N-Biph | H | H | H |
| 2-22-668 | N-Biph | N-Biph | t-bu | t-bu | H |
| 2-22-669 | N-Biph | N-Biph | methyl | methyl | H |
| 2-22-670 | N-Biph | N-Biph | b1 | b1 | H |
| 2-22-671 | N-Biph | N-Biph | b2 | b2 | H |
| 2-22-672 | N-Biph | N-Biph | b3 | b3 | H |
| 2-22-673 | N-Biph | N-Biph | H | H | H |
| 2-22-674 | N-Biph | N-Biph | t-bu | t-bu | H |
| 2-22-675 | N-Biph | N-Biph | methyl | methyl | H |
| 2-22-676 | N-Biph | N-Biph | b1 | b1 | H |
| 2-22-677 | N-Biph | N-Biph | b2 | b2 | H |
| 2-22-678 | N-Biph | N-Biph | b3 | b3 | H |
| 2-22-679 | N-Biph | N-Biph | H | H | H |
| 2-22-680 | N-Biph | N-Biph | t-bu | t-bu | H |
| 2-22-681 | N-Biph | N-Biph | methyl | methyl | H |
| 2-22-682 | N-Biph | N-Biph | b1 | b1 | H |
| 2-22-683 | N-Biph | N-Biph | b2 | b2 | H |
| 2-22-684 | N-Biph | N-Biph | b3 | b3 | H |
| 2-22-685 | N-Biph | N-Biph | H | H | H |
| 2-22-686 | N-Biph | N-Biph | t-bu | t-bu | H |
| 2-22-687 | N-Biph | N-Biph | methyl | methyl | H |
| 2-22-688 | N-Biph | N-Biph | b1 | b1 | H |
| 2-22-689 | N-Biph | N-Biph | b2 | b2 | H |
| 2-22-690 | N-Biph | N-Biph | b3 | b3 | H |
| 2-22-691 | N-Biph | N-Biph | H | H | H |
| 2-22-692 | N-Biph | N-Biph | t-bu | t-bu | H |
| 2-22-693 | N-Biph | N-Biph | methyl | methyl | H |
| 2-22-694 | N-Biph | N-Biph | b1 | b1 | H |
| 2-22-695 | N-Biph | N-Biph | b2 | b2 | H |
| 2-22-696 | N-Biph | N-Biph | b3 | b3 | H |
| 2-22-697 | N-Biph | N-Biph | H | H | H |
| 2-22-698 | N-Biph | N-Biph | t-bu | t-bu | H |
| 2-22-699 | N-Biph | N-Biph | methyl | methyl | H |
| 2-22-700 | N-Biph | N-Biph | b1 | b1 | H |
| 2-22-701 | N-Biph | N-Biph | b2 | b2 | H |
| 2-22-702 | N-Biph | N-Biph | b3 | b3 | H |
| 2-22-703 | N-Biph | N-Biph | H | H | H |
| 2-22-704 | N-Biph | N-Biph | t-bu | t-bu | H |
| 2-22-705 | N-Biph | N-Biph | methyl | methyl | H |
| 2-22-706 | N-Biph | N-Biph | b1 | b1 | H |
| 2-22-707 | N-Biph | N-Biph | b2 | b2 | H |
| 2-22-708 | N-Biph | N-Biph | b3 | b3 | H |
| 2-22-709 | N-Biph | N-Biph | H | H | a1 |
| 2-22-710 | N-Biph | N-Biph | t-bu | t-bu | a1 |
| 2-22-711 | N-Biph | N-Biph | methyl | methyl | a1 |
| 2-22-712 | N-Biph | N-Biph | b1 | b1 | a1 |
| 2-22-713 | N-Biph | N-Biph | b2 | b2 | a1 |
| 2-22-714 | N-Biph | N-Biph | b3 | b3 | a1 |
| 2-22-715 | N-Biph | N-Biph | H | H | a2 |
| 2-22-716 | N-Biph | N-Biph | t-bu | t-bu | a2 |
| 2-22-717 | N-Biph | N-Biph | methyl | methyl | a2 |
| 2-22-718 | N-Biph | N-Biph | b1 | b1 | a2 |
| 2-22-719 | N-Biph | N-Biph | b2 | b2 | a2 |
| 2-22-720 | N-Biph | N-Biph | b3 | b3 | a2 |
| 2-22-721 | N-Biph | N-Biph | H | H | a3 |
| 2-22-722 | N-Biph | N-Biph | t-bu | t-bu | a3 |
| 2-22-723 | N-Biph | N-Biph | methyl | methyl | a3 |
| 2-22-724 | N-Biph | N-Biph | b1 | b1 | a3 |
| 2-22-725 | N-Biph | N-Biph | b2 | b2 | a3 |
| 2-22-726 | N-Biph | N-Biph | b3 | b3 | a3 |
| 2-22-727 | N-Biph | N-Biph | H | H | b1 |
| 2-22-728 | N-Biph | N-Biph | t-bu | t-bu | b1 |
| 2-22-729 | N-Biph | N-Biph | methyl | methyl | b1 |
| 2-22-730 | N-Biph | N-Biph | b1 | b1 | b1 |
| 2-22-731 | N-Biph | N-Biph | b2 | b2 | b1 |
| 2-22-732 | N-Biph | N-Biph | b3 | b3 | b1 |
| 2-22-733 | N-Biph | N-Biph | H | H | b2 |
| 2-22-734 | N-Biph | N-Biph | t-bu | t-bu | b2 |
| 2-22-735 | N-Biph | N-Biph | methyl | methyl | b2 |
| 2-22-736 | N-Biph | N-Biph | b1 | b1 | b2 |
| 2-22-737 | N-Biph | N-Biph | b2 | b2 | b2 |
| 2-22-738 | N-Biph | N-Biph | b3 | b3 | b2 |
| 2-22-739 | N-Biph | N-Biph | H | H | b3 |
| 2-22-740 | N-Biph | N-Biph | t-bu | t-bu | b3 |
| 2-22-741 | N-Biph | N-Biph | methyl | methyl | b3 |
| 2-22-742 | N-Biph | N-Biph | b1 | b1 | b3 |
| 2-22-743 | N-Biph | N-Biph | b2 | b2 | b3 |
| 2-22-744 | N-Biph | N-Biph | b3 | b3 | b3 |
| 2-22-745 | N-Biph | N-Biph | H | H | b5 |
| 2-22-746 | N-Biph | N-Biph | t-bu | t-bu | b5 |
| 2-22-747 | N-Biph | N-Biph | methyl | methyl | b5 |
| 2-22-748 | N-Biph | N-Biph | b1 | b1 | b5 |
| 2-22-749 | N-Biph | N-Biph | b2 | b2 | b5 |
| 2-22-750 | N-Biph | N-Biph | b3 | b3 | b5 |
| 2-22-751 | N-Biph | N-Biph | H | H | b6 |
| 2-22-752 | N-Biph | N-Biph | t-bu | t-bu | b6 |
| 2-22-753 | N-Biph | N-Biph | methyl | methyl | b6 |
| 2-22-754 | N-Biph | N-Biph | b1 | b1 | b6 |
| 2-22-755 | N-Biph | N-Biph | b2 | b2 | b6 |
| 2-22-756 | N-Biph | N-Biph | b3 | b3 | b6 |
| 2-22-757 | N-Biph | N-Biph | H | H | b7 |
| 2-22-758 | N-Biph | N-Biph | t-bu | t-bu | b7 |
| 2-22-759 | N-Biph | N-Biph | methyl | methyl | b7 |
| 2-22-760 | N-Biph | N-Biph | b1 | b1 | b7 |
| 2-22-761 | N-Biph | N-Biph | b2 | b2 | b7 |
| 2-22-762 | N-Biph | N-Biph | b3 | b3 | b7 |
| 2-22-763 | O | N-Ph | H | H | H |
| 2-22-764 | O | N-Ph | t-bu | t-bu | H |
| 2-22-765 | O | N-Ph | methyl | methyl | H |
| 2-22-766 | O | N-Ph | t-bu | t-bu | t-bu |
| 2-22-767 | O | N-Ph | methyl | methyl | methyl |
| 2-22-768 | O | N-Ph | b1 | b1 | H |
| 2-22-769 | O | N-Ph | b2 | b2 | H |
| 2-22-770 | O | N-Ph | b3 | b3 | H |
| 2-22-771 | O | N-Ph | H | H | a1 |
| 2-22-772 | O | N-Ph | t-bu | t-bu | a1 |
| 2-22-773 | O | N-Ph | methyl | methyl | a1 |
| 2-22-774 | O | N-Ph | b1 | b1 | a1 |
| 2-22-775 | O | N-Ph | b2 | b2 | a1 |
| 2-22-776 | O | N-Ph | b3 | b3 | a1 |
| 2-22-777 | O | N-Ph | H | H | a2 |
| 2-22-778 | O | N-Ph | t-bu | t-bu | a2 |
| 2-22-779 | O | N-Ph | methyl | methyl | a2 |
| 2-22-780 | O | N-Ph | b1 | b1 | a2 |
| 2-22-781 | O | N-Ph | b2 | b2 | a2 |
| 2-22-782 | O | N-Ph | b3 | b3 | a2 |
| 2-22-783 | O | N-Ph | H | H | a3 |
| 2-22-784 | O | N-Ph | t-bu | t-bu | a3 |
| 2-22-785 | O | N-Ph | methyl | methyl | a3 |
| 2-22-786 | O | N-Ph | b1 | b1 | a3 |
| 2-22-787 | O | N-Ph | b2 | b2 | a3 |
| 2-22-788 | O | N-Ph | b3 | b3 | a3 |
| 2-22-789 | O | N-Ph | H | H | a4 |
| 2-22-790 | O | N-Ph | t-bu | t-bu | a4 |
| 2-22-791 | O | N-Ph | methyl | methyl | a4 |
| 2-22-792 | O | N-Ph | b1 | b1 | a4 |
| 2-22-793 | O | N-Ph | b2 | b2 | a4 |
| 2-22-794 | O | N-Ph | b3 | b3 | a4 |
| 2-22-795 | O | N-Ph | H | H | a5 |
| 2-22-796 | O | N-Ph | t-bu | t-bu | a5 |
| 2-22-797 | O | N-Ph | methyl | methyl | a5 |
| 2-22-798 | O | N-Ph | b1 | b1 | a5 |
| 2-22-799 | O | N-Ph | b2 | b2 | a5 |
| 2-22-800 | O | N-Ph | b3 | b3 | a5 |
| 2-22-801 | O | N-Ph | H | H | a6 |
| 2-22-802 | O | N-Ph | t-bu | t-bu | a6 |
| 2-22-803 | O | N-Ph | methyl | methyl | a6 |
| 2-22-804 | O | N-Ph | b1 | b1 | a6 |
| 2-22-805 | O | N-Ph | b2 | b2 | a6 |
| 2-22-806 | O | N-Ph | b3 | b3 | a6 |
| 2-22-807 | O | N-Ph | H | H | b1 |
| 2-22-808 | O | N-Ph | t-bu | t-bu | b1 |
| 2-22-809 | O | N-Ph | methyl | methyl | b1 |
| 2-22-810 | O | N-Ph | b1 | b1 | b1 |
| 2-22-811 | O | N-Ph | b2 | b2 | b1 |
| 2-22-812 | O | N-Ph | b3 | b3 | b1 |
| 2-22-813 | O | N-Ph | H | H | b2 |
| 2-22-814 | O | N-Ph | t-bu | t-bu | b2 |
| 2-22-815 | O | N-Ph | methyl | methyl | b2 |
| 2-22-816 | O | N-Ph | b1 | b1 | b2 |
| 2-22-817 | O | N-Ph | b2 | b2 | b2 |
| 2-22-818 | O | N-Ph | b3 | b3 | b2 |
| 2-22-819 | O | N-Ph | H | H | b3 |
| 2-22-820 | O | N-Ph | t-bu | t-bu | b3 |
| 2-22-821 | O | N-Ph | methyl | methyl | b3 |
| 2-22-822 | O | N-Ph | b1 | b1 | b3 |
| 2-22-823 | O | N-Ph | b2 | b2 | b3 |
| 2-22-824 | O | N-Ph | b3 | b3 | b3 |
| 2-22-825 | O | N-Ph | H | H | b4 |
| 2-22-826 | O | N-Ph | t-bu | t-bu | b4 |
| 2-22-827 | O | N-Ph | methyl | methyl | b4 |
| 2-22-828 | O | N-Ph | b1 | b1 | b4 |
| 2-22-829 | O | N-Ph | b2 | b2 | b4 |
| 2-22-830 | O | N-Ph | b3 | b3 | b4 |
| 2-22-831 | O | N-Ph | H | H | b5 |
| 2-22-832 | O | N-Ph | t-bu | t-bu | b5 |
| 2-22-833 | O | N-Ph | methyl | methyl | b5 |
| 2-22-834 | O | N-Ph | b1 | b1 | b5 |
| 2-22-835 | O | N-Ph | b2 | b2 | b5 |
| 2-22-836 | O | N-Ph | b3 | b3 | b5 |
| 2-22-837 | O | N-Ph | H | H | b6 |
| 2-22-838 | O | N-Ph | t-bu | t-bu | b6 |
| 2-22-839 | O | N-Ph | methyl | methyl | b6 |
| 2-22-840 | O | N-Ph | b1 | b1 | b6 |
| 2-22-841 | O | N-Ph | b2 | b2 | b6 |
| 2-22-842 | O | N-Ph | b3 | b3 | b6 |
| 2-22-843 | O | N-Ph | H | H | b7 |
| 2-22-844 | O | N-Ph | t-bu | t-bu | b7 |
| 2-22-845 | O | N-Ph | methyl | methyl | b7 |
| 2-22-846 | O | N-Ph | b1 | b1 | b7 |
| 2-22-847 | O | N-Ph | b2 | b2 | b7 |
| 2-22-848 | O | N-Ph | b3 | b3 | b7 |
| 2-22-849 | O | N-Ph | H | H | b8 |
| 2-22-850 | O | N-Ph | t-bu | t-bu | b8 |
| 2-22-851 | O | N-Ph | methyl | methyl | b8 |
| 2-22-852 | O | N-Ph | b1 | b1 | b8 |
| 2-22-853 | O | N-Ph | b2 | b2 | b8 |
| 2-22-854 | O | N-Ph | b3 | b3 | b8 |
| 2-22-855 | O | N-Ph | H | H | b9 |
| 2-22-856 | O | N-Ph | t-bu | t-bu | b9 |
| 2-22-857 | O | N-Ph | methyl | methyl | b9 |
| 2-22-858 | O | N-Ph | b1 | b1 | b9 |
| 2-22-859 | O | N-Ph | b2 | b2 | b9 |
| 2-22-860 | O | N-Ph | b3 | b3 | b9 |
| 2-22-861 | O | N-Ph | H | H | b10 |
| 2-22-862 | O | N-Ph | t-bu | t-bu | b10 |
| 2-22-863 | O | N-Ph | methyl | methyl | b10 |
| 2-22-864 | O | N-Ph | b1 | b1 | b10 |
| 2-22-865 | O | N-Ph | b2 | b2 | b10 |
| 2-22-866 | O | N-Ph | b3 | b3 | b10 |
| 2-22-867 | O | N-Ph | H | H | H |
| 2-22-868 | O | N-Ph | t-bu | t-bu | H |
| 2-22-869 | O | N-Ph | methyl | methyl | H |
| 2-22-870 | O | N-Ph | b1 | b1 | H |
| 2-22-871 | O | N-Ph | b2 | b2 | H |
| 2-22-872 | O | N-Ph | b3 | b3 | H |
| 2-22-873 | O | N-Ph | H | H | H |
| 2-22-874 | O | N-Ph | t-bu | t-bu | H |
| 2-22-875 | O | N-Ph | methyl | methyl | H |
| 2-22-876 | O | N-Ph | b1 | b1 | H |
| 2-22-877 | O | N-Ph | b2 | b2 | H |
| 2-22-878 | O | N-Ph | b3 | b3 | H |
| 2-22-879 | O | N-Ph | H | H | H |
| 2-22-880 | O | N-Ph | t-bu | t-bu | H |
| 2-22-881 | O | N-Ph | methyl | methyl | H |
| 2-22-882 | O | N-Ph | b1 | b1 | H |
| 2-22-883 | O | N-Ph | b2 | b2 | H |
| 2-22-884 | O | N-Ph | b3 | b3 | H |
| 2-22-885 | O | N-Ph | H | H | H |
| 2-22-886 | O | N-Ph | t-bu | t-bu | H |
| 2-22-887 | O | N-Ph | methyl | methyl | H |
| 2-22-888 | O | N-Ph | b1 | b1 | H |
| 2-22-889 | O | N-Ph | b2 | b2 | H |
| 2-22-890 | O | N-Ph | b3 | b3 | H |
| 2-22-891 | O | N-Ph | H | H | H |
| 2-22-892 | O | N-Ph | t-bu | t-bu | H |
| 2-22-893 | O | N-Ph | methyl | methyl | H |
| 2-22-894 | O | N-Ph | b1 | b1 | H |
| 2-22-895 | O | N-Ph | b2 | b2 | H |
| 2-22-896 | O | N-Ph | b3 | b3 | H |
| 2-22-897 | O | N-Ph | H | H | H |
| 2-22-898 | O | N-Ph | t-bu | t-bu | H |
| 2-22-899 | O | N-Ph | methyl | methyl | H |
| 2-22-900 | O | N-Ph | b1 | b1 | H |
| 2-22-901 | O | N-Ph | b2 | b2 | H |
| 2-22-902 | O | N-Ph | b3 | b3 | H |
| 2-22-903 | O | N-Ph | H | H | H |
| 2-22-904 | O | N-Ph | t-bu | t-bu | H |
| 2-22-905 | O | N-Ph | methyl | methyl | H |
| 2-22-906 | O | N-Ph | b1 | b1 | H |
| 2-22-907 | O | N-Ph | b2 | b2 | H |
| 2-22-908 | O | N-Ph | b3 | b3 | H |
| 2-22-909 | O | N-Ph | H | H | H |
| 2-22-910 | O | N-Ph | t-bu | t-bu | H |
| 2-22-911 | O | N-Ph | methyl | methyl | H |
| 2-22-912 | O | N-Ph | b1 | b1 | H |
| 2-22-913 | O | N-Ph | b2 | b2 | H |
| 2-22-914 | O | N-Ph | b3 | b3 | H |
| 2-22-915 | O | N-Ph | H | H | H |
| 2-22-916 | O | N-Ph | t-bu | t-bu | H |
| 2-22-917 | O | N-Ph | methyl | methyl | H |
| 2-22-918 | O | N-Ph | b1 | b1 | H |
| 2-22-919 | O | N-Ph | b2 | b2 | H |
| 2-22-920 | O | N-Ph | b3 | b3 | H |
| 2-22-921 | O | N-Ph | H | H | H |
| 2-22-922 | O | N-Ph | t-bu | t-bu | H |
| 2-22-923 | O | N-Ph | methyl | methyl | H |
| 2-22-924 | O | N-Ph | b1 | b1 | H |
| 2-22-925 | O | N-Ph | b2 | b2 | H |
| 2-22-926 | O | N-Ph | b3 | b3 | H |
| 2-22-927 | O | N-Ph | H | H | H |
| 2-22-928 | O | N-Ph | t-bu | t-bu | H |
| 2-22-929 | O | N-Ph | methyl | methyl | H |
| 2-22-930 | O | N-Ph | b1 | b1 | H |
| 2-22-931 | O | N-Ph | b2 | b2 | H |
| 2-22-932 | O | N-Ph | b3 | b3 | H |
| 2-22-933 | O | N-Ph | H | H | H |
| 2-22-934 | O | N-Ph | t-bu | t-bu | H |
| 2-22-935 | O | N-Ph | methyl | methyl | H |
| 2-22-936 | O | N-Ph | b1 | b1 | H |
| 2-22-937 | O | N-Ph | b2 | b2 | H |
| 2-22-938 | O | N-Ph | b3 | b3 | H |
| 2-22-939 | O | N-Ph | H | H | H |
| 2-22-940 | O | N-Ph | t-bu | t-bu | H |
| 2-22-941 | O | N-Ph | methyl | methyl | H |
| 2-22-942 | O | N-Ph | b1 | b1 | H |
| 2-22-943 | O | N-Ph | b2 | b2 | H |
| 2-22-944 | O | N-Ph | b3 | b3 | H |
| 2-22-945 | O | N-Ph | H | H | H |
| 2-22-946 | O | N-Ph | t-bu | t-bu | H |
| 2-22-947 | O | N-Ph | methyl | methyl | H |
| 2-22-948 | O | N-Ph | b1 | b1 | H |
| 2-22-949 | O | N-Ph | b2 | b2 | H |
| 2-22-950 | O | N-Ph | b3 | b3 | H |
| 2-22-951 | O | N-Ph | H | H | H |
| 2-22-952 | O | N-Ph | t-bu | t-bu | H |
| 2-22-953 | O | N-Ph | methyl | methyl | H |
| 2-22-954 | O | N-Ph | b1 | b1 | H |
| 2-22-955 | O | N-Ph | b2 | b2 | H |
| 2-22-956 | O | N-Ph | b3 | b3 | H |
| 2-22-957 | O | N-Ph | H | H | H |
| 2-22-958 | O | N-Ph | t-bu | t-bu | H |
| 2-22-959 | O | N-Ph | methyl | methyl | H |
| 2-22-960 | O | N-Ph | b1 | b1 | H |
| 2-22-961 | O | N-Ph | b2 | b2 | H |
| 2-22-962 | O | N-Ph | b3 | b3 | H |
| 2-22-963 | O | N-Ph | H | H | a1 |
| 2-22-964 | O | N-Ph | t-bu | t-bu | a1 |
| 2-22-965 | O | N-Ph | methyl | methyl | a1 |
| 2-22-966 | O | N-Ph | b1 | b1 | a1 |
| 2-22-967 | O | N-Ph | b2 | b2 | a1 |
| 2-22-968 | O | N-Ph | b3 | b3 | a1 |
| 2-22-969 | O | N-Ph | H | H | a2 |
| 2-22-970 | O | N-Ph | t-bu | t-bu | a2 |
| 2-22-971 | O | N-Ph | methyl | methyl | a2 |
| 2-22-972 | O | N-Ph | b1 | b1 | a2 |
| 2-22-973 | O | N-Ph | b2 | b2 | a2 |
| 2-22-974 | O | N-Ph | b3 | b3 | a2 |
| 2-22-975 | O | N-Ph | H | H | a3 |
| 2-22-976 | O | N-Ph | t-bu | t-bu | a3 |
| 2-22-977 | O | N-Ph | methyl | methyl | a3 |
| 2-22-978 | O | N-Ph | b1 | b1 | a3 |
| 2-22-979 | O | N-Ph | b2 | b2 | a3 |
| 2-22-980 | O | N-Ph | b3 | b3 | a3 |
| 2-22-981 | O | N-Ph | H | H | b1 |
| 2-22-982 | O | N-Ph | t-bu | t-bu | b1 |
| 2-22-983 | O | N-Ph | methyl | methyl | b1 |
| 2-22-984 | O | N-Ph | b1 | b1 | b1 |
| 2-22-985 | O | N-Ph | b2 | b2 | b1 |
| 2-22-986 | O | N-Ph | b3 | b3 | b1 |
| 2-22-987 | O | N-Ph | H | H | b2 |
| 2-22-988 | O | N-Ph | t-bu | t-bu | b2 |
| 2-22-989 | O | N-Ph | methyl | methyl | b2 |
| 2-22-990 | O | N-Ph | b1 | b1 | b2 |
| 2-22-991 | O | N-Ph | b2 | b2 | b2 |
| 2-22-992 | O | N-Ph | b3 | b3 | b2 |
| 2-22-993 | O | N-Ph | H | H | b3 |
| 2-22-994 | O | N-Ph | t-bu | t-bu | b3 |
| 2-22-995 | O | N-Ph | methyl | methyl | b3 |
| 2-22-996 | O | N-Ph | b1 | b1 | b3 |
| 2-22-997 | O | N-Ph | b2 | b2 | b3 |
| 2-22-998 | O | N-Ph | b3 | b3 | b3 |
| 2-22-999 | O | N-Ph | H | H | b5 |
| 2-22-1000 | O | N-Ph | t-bu | t-bu | b5 |
| 2-22-1001 | O | N-Ph | methyl | methyl | b5 |
| 2-22-1002 | O | N-Ph | b1 | b1 | b5 |
| 2-22-1003 | O | N-Ph | b2 | b2 | b5 |
| 2-22-1004 | O | N-Ph | b3 | b3 | b5 |
| 2-22-1005 | O | N-Ph | H | H | b6 |
| 2-22-1006 | O | N-Ph | t-bu | t-bu | b6 |
| 2-22-1007 | O | N-Ph | methyl | methyl | b6 |
| 2-22-1008 | O | N-Ph | b1 | b1 | b6 |
| 2-22-1009 | O | N-Ph | b2 | b2 | b6 |
| 2-22-1010 | O | N-Ph | b3 | b3 | b6 |
| 2-22-1011 | O | N-Ph | H | H | b7 |
| 2-22-1012 | O | N-Ph | t-bu | t-bu | b7 |
| 2-22-1013 | O | N-Ph | methyl | methyl | b7 |
| 2-22-1014 | O | N-Ph | b1 | b1 | b7 |
| 2-22-1015 | O | N-Ph | b2 | b2 | b7 |
| 2-22-1016 | O | N-Ph | b3 | b3 | b7 |
| 2-22-1017 | S | N-Ph | H | H | H |
| 2-22-1018 | S | N-Ph | t-bu | t-bu | H |
| 2-22-1019 | S | N-Ph | methyl | methyl | H |
| 2-22-1020 | S | N-Ph | t-bu | t-bu | t-bu |
| 2-22-1021 | S | N-Ph | methyl | methyl | methyl |
| 2-22-1022 | S | N-Ph | b1 | b1 | H |
| 2-22-1023 | S | N-Ph | b2 | b2 | H |
| 2-22-1024 | S | N-Ph | b3 | b3 | H |
| 2-22-1025 | S | N-Ph | H | H | a1 |
| 2-22-1026 | S | N-Ph | t-bu | t-bu | a1 |
| 2-22-1027 | S | N-Ph | methyl | methyl | a1 |
| 2-22-1028 | S | N-Ph | b1 | b1 | a1 |
| 2-22-1029 | S | N-Ph | b2 | b2 | a1 |
| 2-22-1030 | S | N-Ph | b3 | b3 | a1 |
| 2-22-1031 | S | N-Ph | H | H | a2 |
| 2-22-1032 | S | N-Ph | t-bu | t-bu | a2 |
| 2-22-1033 | S | N-Ph | methyl | methyl | a2 |
| 2-22-1034 | S | N-Ph | b1 | b1 | a2 |
| 2-22-1035 | S | N-Ph | b2 | b2 | a2 |
| 2-22-1036 | S | N-Ph | b3 | b3 | a2 |
| 2-22-1037 | S | N-Ph | H | H | a3 |
| 2-22-1038 | S | N-Ph | t-bu | t-bu | a3 |
| 2-22-1039 | S | N-Ph | methyl | methyl | a3 |
| 2-22-1040 | S | N-Ph | b1 | b1 | a3 |
| 2-22-1041 | S | N-Ph | b2 | b2 | a3 |
| 2-22-1042 | S | N-Ph | b3 | b3 | a3 |
| 2-22-1043 | S | N-Ph | H | H | a4 |
| 2-22-1044 | S | N-Ph | t-bu | t-bu | a4 |
| 2-22-1045 | S | N-Ph | methyl | methyl | a4 |
| 2-22-1046 | S | N-Ph | b1 | b1 | a4 |
| 2-22-1047 | S | N-Ph | b2 | b2 | a4 |
| 2-22-1048 | S | N-Ph | b3 | b3 | a4 |
| 2-22-1049 | S | N-Ph | H | H | a5 |
| 2-22-1050 | S | N-Ph | t-bu | t-bu | a5 |
| 2-22-1051 | S | N-Ph | methyl | methyl | a5 |
| 2-22-1052 | S | N-Ph | b1 | b1 | a5 |
| 2-22-1053 | S | N-Ph | b2 | b2 | a5 |
| 2-22-1054 | S | N-Ph | b3 | b3 | a5 |
| 2-22-1055 | S | N-Ph | H | H | a6 |
| 2-22-1056 | S | N-Ph | t-bu | t-bu | a6 |
| 2-22-1057 | S | N-Ph | methyl | methyl | a6 |
| 2-22-1058 | S | N-Ph | b1 | b1 | a6 |
| 2-22-1059 | S | N-Ph | b2 | b2 | a6 |
| 2-22-1060 | S | N-Ph | b3 | b3 | a6 |
| 2-22-1061 | S | N-Ph | H | H | b1 |
| 2-22-1062 | S | N-Ph | t-bu | t-bu | b1 |
| 2-22-1063 | S | N-Ph | methyl | methyl | b1 |
| 2-22-1064 | S | N-Ph | b1 | b1 | b1 |
| 2-22-1065 | S | N-Ph | b2 | b2 | b1 |
| 2-22-1066 | S | N-Ph | b3 | b3 | b1 |
| 2-22-1067 | S | N-Ph | H | H | b2 |
| 2-22-1068 | S | N-Ph | t-bu | t-bu | b2 |
| 2-22-1069 | S | N-Ph | methyl | methyl | b2 |
| 2-22-1070 | S | N-Ph | b1 | b1 | b2 |
| 2-22-1071 | S | N-Ph | b2 | b2 | b2 |
| 2-22-1072 | S | N-Ph | b3 | b3 | b2 |
| 2-22-1073 | S | N-Ph | H | H | b3 |
| 2-22-1074 | S | N-Ph | t-bu | t-bu | b3 |
| 2-22-1075 | S | N-Ph | methyl | methyl | b3 |
| 2-22-1076 | S | N-Ph | b1 | b1 | b3 |
| 2-22-1077 | S | N-Ph | b2 | b2 | b3 |
| 2-22-1078 | S | N-Ph | b3 | b3 | b3 |
| 2-22-1079 | S | N-Ph | H | H | b4 |
| 2-22-1080 | S | N-Ph | t-bu | t-bu | b4 |
| 2-22-1081 | S | N-Ph | methyl | methyl | b4 |
| 2-22-1082 | S | N-Ph | b1 | b1 | b4 |
| 2-22-1083 | S | N-Ph | b2 | b2 | b4 |
| 2-22-1084 | S | N-Ph | b3 | b3 | b4 |
| 2-22-1085 | S | N-Ph | H | H | b5 |
| 2-22-1086 | S | N-Ph | t-bu | t-bu | b5 |
| 2-22-1087 | S | N-Ph | methyl | methyl | b5 |
| 2-22-1088 | S | N-Ph | b1 | b1 | b5 |
| 2-22-1089 | S | N-Ph | b2 | b2 | b5 |
| 2-22-1090 | S | N-Ph | b3 | b3 | b5 |
| 2-22-1091 | S | N-Ph | H | H | b6 |
| 2-22-1092 | S | N-Ph | t-bu | t-bu | b6 |
| 2-22-1093 | S | N-Ph | methyl | methyl | b6 |
| 2-22-1094 | S | N-Ph | b1 | b1 | b6 |
| 2-22-1095 | S | N-Ph | b2 | b2 | b6 |
| 2-22-1096 | S | N-Ph | b3 | b3 | b6 |
| 2-22-1097 | S | N-Ph | H | H | b7 |
| 2-22-1098 | S | N-Ph | t-bu | t-bu | b7 |
| 2-22-1099 | S | N-Ph | methyl | methyl | b7 |
| 2-22-1100 | S | N-Ph | b1 | b1 | b7 |
| 2-22-1101 | S | N-Ph | b2 | b2 | b7 |
| 2-22-1102 | S | N-Ph | b3 | b3 | b7 |
| 2-22-1103 | S | N-Ph | H | H | b8 |
| 2-22-1104 | S | N-Ph | t-bu | t-bu | b8 |
| 2-22-1105 | S | N-Ph | methyl | methyl | b8 |
| 2-22-1106 | S | N-Ph | b1 | b1 | b8 |
| 2-22-1107 | S | N-Ph | b2 | b2 | b8 |
| 2-22-1108 | S | N-Ph | b3 | b3 | b8 |
| 2-22-1109 | S | N-Ph | H | H | b9 |
| 2-22-1110 | S | N-Ph | t-bu | t-bu | b9 |
| 2-22-1111 | S | N-Ph | methyl | methyl | b9 |
| 2-22-1112 | S | N-Ph | b1 | b1 | b9 |
| 2-22-1113 | S | N-Ph | b2 | b2 | b9 |
| 2-22-1114 | S | N-Ph | b3 | b3 | b9 |
| 2-22-1115 | S | N-Ph | H | H | b10 |
| 2-22-1116 | S | N-Ph | t-bu | t-bu | b10 |
| 2-22-1117 | S | N-Ph | methyl | methyl | b10 |
| 2-22-1118 | S | N-Ph | b1 | b1 | b10 |
| 2-22-1119 | S | N-Ph | b2 | b2 | b10 |
| 2-22-1120 | S | N-Ph | b3 | b3 | b10 |
| 2-22-1121 | S | N-Ph | H | H | H |
| 2-22-1122 | S | N-Ph | t-bu | t-bu | H |
| 2-22-1123 | S | N-Ph | methyl | methyl | H |
| 2-22-1124 | S | N-Ph | b1 | b1 | H |
| 2-22-1125 | S | N-Ph | b2 | b2 | H |
| 2-22-1126 | S | N-Ph | b3 | b3 | H |
| 2-22-1127 | S | N-Ph | H | H | H |
| 2-22-1128 | S | N-Ph | t-bu | t-bu | H |
| 2-22-1129 | S | N-Ph | methyl | methyl | H |
| 2-22-1130 | S | N-Ph | b1 | b1 | H |
| 2-22-1131 | S | N-Ph | b2 | b2 | H |
| 2-22-1132 | S | N-Ph | b3 | b3 | H |
| 2-22-1133 | S | N-Ph | H | H | H |
| 2-22-1134 | S | N-Ph | t-bu | t-bu | H |
| 2-22-1135 | S | N-Ph | methyl | methyl | H |
| 2-22-1136 | S | N-Ph | b1 | b1 | H |
| 2-22-1137 | S | N-Ph | b2 | b2 | H |
| 2-22-1138 | S | N-Ph | b3 | b3 | H |
| 2-22-1139 | S | N-Ph | H | H | H |
| 2-22-1140 | S | N-Ph | t-bu | t-bu | H |
| 2-22-1141 | S | N-Ph | methyl | methyl | H |
| 2-22-1142 | S | N-Ph | b1 | b1 | H |
| 2-22-1143 | S | N-Ph | b2 | b2 | H |
| 2-22-1144 | S | N-Ph | b3 | b3 | H |
| 2-22-1145 | S | N-Ph | H | H | H |
| 2-22-1146 | S | N-Ph | t-bu | t-bu | H |
| 2-22-1147 | S | N-Ph | methyl | methyl | H |
| 2-22-1148 | S | N-Ph | b1 | b1 | H |
| 2-22-1149 | S | N-Ph | b2 | b2 | H |
| 2-22-1150 | S | N-Ph | b3 | b3 | H |
| 2-22-1151 | S | N-Ph | H | H | H |
| 2-22-1152 | S | N-Ph | t-bu | t-bu | H |
| 2-22-1153 | S | N-Ph | methyl | methyl | H |
| 2-22-1154 | S | N-Ph | b1 | b1 | H |
| 2-22-1155 | S | N-Ph | b2 | b2 | H |
| 2-22-1156 | S | N-Ph | b3 | b3 | H |
| 2-22-1157 | S | N-Ph | H | H | H |
| 2-22-1158 | S | N-Ph | t-bu | t-bu | H |
| 2-22-1159 | S | N-Ph | methyl | methyl | H |
| 2-22-1160 | S | N-Ph | b1 | b1 | H |
| 2-22-1161 | S | N-Ph | b2 | b2 | H |
| 2-22-1162 | S | N-Ph | b3 | b3 | H |
| 2-22-1163 | S | N-Ph | H | H | H |
| 2-22-1164 | S | N-Ph | t-bu | t-bu | H |
| 2-22-1165 | S | N-Ph | methyl | methyl | H |
| 2-22-1166 | S | N-Ph | b1 | b1 | H |
| 2-22-1167 | S | N-Ph | b2 | b2 | H |
| 2-22-1168 | S | N-Ph | b3 | b3 | H |
| 2-22-1169 | S | N-Ph | H | H | H |
| 2-22-1170 | S | N-Ph | t-bu | t-bu | H |
| 2-22-1171 | S | N-Ph | methyl | methyl | H |
| 2-22-1172 | S | N-Ph | b1 | b1 | H |
| 2-22-1173 | S | N-Ph | b2 | b2 | H |
| 2-22-1174 | S | N-Ph | b3 | b3 | H |
| 2-22-1175 | S | N-Ph | H | H | H |
| 2-22-1176 | S | N-Ph | t-bu | t-bu | H |
| 2-22-1177 | S | N-Ph | methyl | methyl | H |
| 2-22-1178 | S | N-Ph | b1 | b1 | H |
| 2-22-1179 | S | N-Ph | b2 | b2 | H |
| 2-22-1180 | S | N-Ph | b3 | b3 | H |
| 2-22-1181 | S | N-Ph | H | H | H |
| 2-22-1182 | S | N-Ph | t-bu | t-bu | H |
| 2-22-1183 | S | N-Ph | methyl | methyl | H |
| 2-22-1184 | S | N-Ph | b1 | b1 | H |
| 2-22-1185 | S | N-Ph | b2 | b2 | H |
| 2-22-1186 | S | N-Ph | b3 | b3 | H |
| 2-22-1187 | S | N-Ph | H | H | H |
| 2-22-1188 | S | N-Ph | t-bu | t-bu | H |
| 2-22-1189 | S | N-Ph | methyl | methyl | H |
| 2-22-1190 | S | N-Ph | b1 | b1 | H |
| 2-22-1191 | S | N-Ph | b2 | b2 | H |
| 2-22-1192 | S | N-Ph | b3 | b3 | H |
| 2-22-1193 | S | N-Ph | H | H | H |
| 2-22-1194 | S | N-Ph | t-bu | t-bu | H |
| 2-22-1195 | S | N-Ph | methyl | methyl | H |
| 2-22-1196 | S | N-Ph | b1 | b1 | H |
| 2-22-1197 | S | N-Ph | b2 | b2 | H |
| 2-22-1198 | S | N-Ph | b3 | b3 | H |
| 2-22-1199 | S | N-Ph | H | H | H |
| 2-22-1200 | S | N-Ph | t-bu | t-bu | H |
| 2-22-1201 | S | N-Ph | methyl | methyl | H |
| 2-22-1202 | S | N-Ph | b1 | b1 | H |
| 2-22-1203 | S | N-Ph | b2 | b2 | H |
| 2-22-1204 | S | N-Ph | b3 | b3 | H |
| 2-22-1205 | S | N-Ph | H | H | H |
| 2-22-1206 | S | N-Ph | t-bu | t-bu | H |
| 2-22-1207 | S | N-Ph | methyl | methyl | H |
| 2-22-1208 | S | N-Ph | b1 | b1 | H |
| 2-22-1209 | S | N-Ph | b2 | b2 | H |
| 2-22-1210 | S | N-Ph | b3 | b3 | H |
| 2-22-1211 | S | N-Ph | H | H | H |
| 2-22-1212 | S | N-Ph | t-bu | t-bu | H |
| 2-22-1213 | S | N-Ph | methyl | methyl | H |
| 2-22-1214 | S | N-Ph | b1 | b1 | H |
| 2-22-1215 | S | N-Ph | b2 | b2 | H |
| 2-22-1216 | S | N-Ph | b3 | b3 | H |
| 2-22-1217 | S | N-Ph | H | H | a1 |
| 2-22-1218 | S | N-Ph | t-bu | t-bu | a1 |
| 2-22-1219 | S | N-Ph | methyl | methyl | a1 |
| 2-22-1220 | S | N-Ph | b1 | b1 | a1 |
| 2-22-1221 | S | N-Ph | b2 | b2 | a1 |
| 2-22-1222 | S | N-Ph | b3 | b3 | a1 |
| 2-22-1223 | S | N-Ph | H | H | a2 |
| 2-22-1224 | S | N-Ph | t-bu | t-bu | a2 |
| 2-22-1225 | S | N-Ph | methyl | methyl | a2 |
| 2-22-1226 | S | N-Ph | b1 | b1 | a2 |
| 2-22-1227 | S | N-Ph | b2 | b2 | a2 |
| 2-22-1228 | S | N-Ph | b3 | b3 | a2 |
| 2-22-1229 | S | N-Ph | H | H | a3 |
| 2-22-1230 | S | N-Ph | t-bu | t-bu | a3 |
| 2-22-1231 | S | N-Ph | methyl | methyl | a3 |
| 2-22-1232 | S | N-Ph | b1 | b1 | a3 |
| 2-22-1233 | S | N-Ph | b2 | b2 | a3 |
| 2-22-1234 | S | N-Ph | b3 | b3 | a3 |
| 2-22-1235 | S | N-Ph | H | H | b1 |
| 2-22-1236 | S | N-Ph | t-bu | t-bu | b1 |
| 2-22-1237 | S | N-Ph | methyl | methyl | b1 |
| 2-22-1238 | S | N-Ph | b1 | b1 | b1 |
| 2-22-1239 | S | N-Ph | b2 | b2 | b1 |
| 2-22-1240 | S | N-Ph | b3 | b3 | b1 |
| 2-22-1241 | S | N-Ph | H | H | b2 |
| 2-22-1242 | S | N-Ph | t-bu | t-bu | b2 |
| 2-22-1243 | S | N-Ph | methyl | methyl | b2 |
| 2-22-1244 | S | N-Ph | b1 | b1 | b2 |
| 2-22-1245 | S | N-Ph | b2 | b2 | b2 |
| 2-22-1246 | S | N-Ph | b3 | b3 | b2 |
| 2-22-1247 | S | N-Ph | H | H | b3 |
| 2-22-1248 | S | N-Ph | t-bu | t-bu | b3 |
| 2-22-1249 | S | N-Ph | methyl | methyl | b3 |
| 2-22-1250 | S | N-Ph | b1 | b1 | b3 |
| 2-22-1251 | S | N-Ph | b2 | b2 | b3 |
| 2-22-1252 | S | N-Ph | b3 | b3 | b3 |
| 2-22-1253 | S | N-Ph | H | H | b5 |
| 2-22-1254 | S | N-Ph | t-bu | t-bu | b5 |
| 2-22-1255 | S | N-Ph | methyl | methyl | b5 |
| 2-22-1256 | S | N-Ph | b1 | b1 | b5 |
| 2-22-1257 | S | N-Ph | b2 | b2 | b5 |
| 2-22-1258 | S | N-Ph | b3 | b3 | b5 |
| 2-22-1259 | S | N-Ph | H | H | b6 |
| 2-22-1260 | S | N-Ph | t-bu | t-bu | b6 |
| 2-22-1261 | S | N-Ph | methyl | methyl | b6 |
| 2-22-1262 | S | N-Ph | b1 | b1 | b6 |
| 2-22-1263 | S | N-Ph | b2 | b2 | b6 |
| 2-22-1264 | S | N-Ph | b3 | b3 | b6 |
| 2-22-1265 | S | N-Ph | H | H | b7 |
| 2-22-1266 | S | N-Ph | t-bu | t-bu | b7 |
| 2-22-1267 | S | N-Ph | methyl | methyl | b7 |
| 2-22-1268 | S | N-Ph | b1 | b1 | b7 |
| 2-22-1269 | S | N-Ph | b2 | b2 | b7 |
| 2-22-1270 | S | N-Ph | b3 | b3 | b7 |

### Group 2-23

The compounds are represented by the formula 2-23 below, wherein CY₁ is a benzene group X₁ is B, and Y1, Y2, Y3, R2b, R3a, (R1)a1 are each shown in the table 23 below,

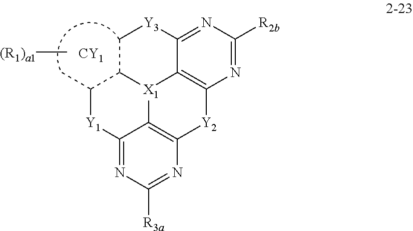

TABLE 23

| Compound | | | | | |
|---|---|---|---|---|---|
| No. | Y1/Y3 | Y2 | R2b | R3a | (R1)a1 |
| 2-23-001 | N-Ph | N-Ph | H | H | H |
| 2-23-002 | N-Ph | N-Ph | t-bu | t-bu | H |
| 2-23-003 | N-Ph | N-Ph | methyl | methyl | H |
| 2-23-004 | N-Ph | N-Ph | t-bu | t-bu | t-bu |
| 2-23-005 | N-Ph | N-Ph | methyl | methyl | methyl |
| 2-23-006 | N-Ph | N-Ph | b1 | b1 | H |
| 2-23-007 | N-Ph | N-Ph | b2 | b2 | H |
| 2-23-008 | N-Ph | N-Ph | b3 | b3 | H |
| 2-23-009 | N-Ph | N-Ph | H | H | a1 |
| 2-23-010 | N-Ph | N-Ph | t-bu | t-bu | a1 |
| 2-23-011 | N-Ph | N-Ph | methyl | methyl | a1 |
| 2-23-012 | N-Ph | N-Ph | b1 | b1 | a1 |
| 2-23-013 | N-Ph | N-Ph | b2 | b2 | a1 |
| 2-23-014 | N-Ph | N-Ph | b3 | b3 | a1 |
| 2-23-015 | N-Ph | N-Ph | H | H | a2 |
| 2-23-016 | N-Ph | N-Ph | t-bu | t-bu | a2 |
| 2-23-017 | N-Ph | N-Ph | methyl | methyl | a2 |
| 2-23-018 | N-Ph | N-Ph | b1 | b1 | a2 |
| 2-23-019 | N-Ph | N-Ph | b2 | b2 | a2 |
| 2-23-020 | N-Ph | N-Ph | b3 | b3 | a2 |
| 2-23-021 | N-Ph | N-Ph | H | H | a3 |
| 2-23-022 | N-Ph | N-Ph | t-bu | t-bu | a3 |
| 2-23-023 | N-Ph | N-Ph | methyl | methyl | a3 |
| 2-23-024 | N-Ph | N-Ph | b1 | b1 | a3 |
| 2-23-025 | N-Ph | N-Ph | b2 | b2 | a3 |
| 2-23-026 | N-Ph | N-Ph | b3 | b3 | a3 |
| 2-23-027 | N-Ph | N-Ph | H | H | a4 |
| 2-23-028 | N-Ph | N-Ph | t-bu | t-bu | a4 |
| 2-23-029 | N-Ph | N-Ph | methyl | methyl | a4 |
| 2-23-030 | N-Ph | N-Ph | b1 | b1 | a4 |
| 2-23-031 | N-Ph | N-Ph | b2 | b2 | a4 |
| 2-23-032 | N-Ph | N-Ph | b3 | b3 | a4 |
| 2-23-033 | N-Ph | N-Ph | H | H | a5 |
| 2-23-034 | N-Ph | N-Ph | t-bu | t-bu | a5 |
| 2-23-035 | N-Ph | N-Ph | methyl | methyl | a5 |
| 2-23-036 | N-Ph | N-Ph | b1 | b1 | a5 |
| 2-23-037 | N-Ph | N-Ph | b2 | b2 | a5 |
| 2-23-038 | N-Ph | N-Ph | b3 | b3 | a5 |
| 2-23-039 | N-Ph | N-Ph | H | H | a6 |
| 2-23-040 | N-Ph | N-Ph | t-bu | t-bu | a6 |
| 2-23-041 | N-Ph | N-Ph | methyl | methyl | a6 |
| 2-23-042 | N-Ph | N-Ph | b1 | b1 | a6 |
| 2-23-043 | N-Ph | N-Ph | b2 | b2 | a6 |
| 2-23-044 | N-Ph | N-Ph | b3 | b3 | a6 |
| 2-23-045 | N-Ph | N-Ph | H | H | b1 |
| 2-23-046 | N-Ph | N-Ph | t-bu | t-bu | b1 |
| 2-23-047 | N-Ph | N-Ph | methyl | methyl | b1 |
| 2-23-048 | N-Ph | N-Ph | b1 | b1 | b1 |
| 2-23-049 | N-Ph | N-Ph | b2 | b2 | b1 |
| 2-23-050 | N-Ph | N-Ph | b3 | b3 | b1 |
| 2-23-051 | N-Ph | N-Ph | H | H | b2 |
| 2-23-052 | N-Ph | N-Ph | t-bu | t-bu | b2 |
| 2-23-053 | N-Ph | N-Ph | methyl | methyl | b2 |
| 2-23-054 | N-Ph | N-Ph | b1 | b1 | b2 |
| 2-23-055 | N-Ph | N-Ph | b2 | b2 | b2 |
| 2-23-056 | N-Ph | N-Ph | b3 | b3 | b2 |
| 2-23-057 | N-Ph | N-Ph | H | H | b3 |
| 2-23-058 | N-Ph | N-Ph | t-bu | t-bu | b3 |
| 2-23-059 | N-Ph | N-Ph | methyl | methyl | b3 |
| 2-23-060 | N-Ph | N-Ph | b1 | b1 | b3 |
| 2-23-061 | N-Ph | N-Ph | b2 | b2 | b3 |
| 2-23-062 | N-Ph | N-Ph | b3 | b3 | b3 |
| 2-23-063 | N-Ph | N-Ph | H | H | b4 |
| 2-23-064 | N-Ph | N-Ph | t-bu | t-bu | b4 |
| 2-23-065 | N-Ph | N-Ph | methyl | methyl | b4 |
| 2-23-066 | N-Ph | N-Ph | b1 | b1 | b4 |
| 2-23-067 | N-Ph | N-Ph | b2 | b2 | b4 |
| 2-23-068 | N-Ph | N-Ph | b3 | b3 | b4 |
| 2-23-069 | N-Ph | N-Ph | H | H | b5 |
| 2-23-070 | N-Ph | N-Ph | t-bu | t-bu | b5 |
| 2-23-071 | N-Ph | N-Ph | methyl | methyl | b5 |
| 2-23-072 | N-Ph | N-Ph | b1 | b1 | b5 |
| 2-23-073 | N-Ph | N-Ph | b2 | b2 | b5 |
| 2-23-074 | N-Ph | N-Ph | b3 | b3 | b5 |
| 2-23-075 | N-Ph | N-Ph | H | H | b6 |
| 2-23-076 | N-Ph | N-Ph | t-bu | t-bu | b6 |
| 2-23-077 | N-Ph | N-Ph | methyl | methyl | b6 |
| 2-23-078 | N-Ph | N-Ph | b1 | b1 | b6 |
| 2-23-079 | N-Ph | N-Ph | b2 | b2 | b6 |
| 2-23-080 | N-Ph | N-Ph | b3 | b3 | b6 |
| 2-23-081 | N-Ph | N-Ph | H | H | b7 |
| 2-23-082 | N-Ph | N-Ph | t-bu | t-bu | b7 |
| 2-23-083 | N-Ph | N-Ph | methyl | methyl | b7 |
| 2-23-084 | N-Ph | N-Ph | b1 | b1 | b7 |
| 2-23-085 | N-Ph | N-Ph | b2 | b2 | b7 |
| 2-23-086 | N-Ph | N-Ph | b3 | b3 | b7 |
| 2-23-087 | N-Ph | N-Ph | H | H | b8 |
| 2-23-088 | N-Ph | N-Ph | t-bu | t-bu | b8 |
| 2-23-089 | N-Ph | N-Ph | methyl | methyl | b8 |
| 2-23-090 | N-Ph | N-Ph | b1 | b1 | b8 |
| 2-23-091 | N-Ph | N-Ph | b2 | b2 | b8 |
| 2-23-092 | N-Ph | N-Ph | b3 | b3 | b8 |
| 2-23-093 | N-Ph | N-Ph | H | H | b9 |
| 2-23-094 | N-Ph | N-Ph | t-bu | t-bu | b9 |
| 2-23-095 | N-Ph | N-Ph | methyl | methyl | b9 |
| 2-23-096 | N-Ph | N-Ph | b1 | b1 | b9 |
| 2-23-097 | N-Ph | N-Ph | b2 | b2 | b9 |
| 2-23-098 | N-Ph | N-Ph | b3 | b3 | b9 |
| 2-23-099 | N-Ph | N-Ph | H | H | b10 |
| 2-23-100 | N-Ph | N-Ph | t-bu | t-bu | b10 |
| 2-23-101 | N-Ph | N-Ph | methyl | methyl | b10 |
| 2-23-102 | N-Ph | N-Ph | b1 | b1 | b10 |
| 2-23-103 | N-Ph | N-Ph | b2 | b2 | b10 |
| 2-23-104 | N-Ph | N-Ph | b3 | b3 | b10 |
| 2-23-105 | N-Ph | N-Ph | H | H | H |
| 2-23-106 | N-Ph | N-Ph | t-bu | t-bu | H |
| 2-23-107 | N-Ph | N-Ph | methyl | methyl | H |
| 2-23-108 | N-Ph | N-Ph | b1 | b1 | H |
| 2-23-109 | N-Ph | N-Ph | b2 | b2 | H |
| 2-23-110 | N-Ph | N-Ph | b3 | b3 | H |
| 2-23-111 | N-Ph | N-Ph | H | H | H |
| 2-23-112 | N-Ph | N-Ph | t-bu | t-bu | H |
| 2-23-113 | N-Ph | N-Ph | methyl | methyl | H |
| 2-23-114 | N-Ph | N-Ph | b1 | b1 | H |
| 2-23-115 | N-Ph | N-Ph | b2 | b2 | H |
| 2-23-116 | N-Ph | N-Ph | b3 | b3 | H |
| 2-23-117 | N-Ph | N-Ph | H | H | H |
| 2-23-118 | N-Ph | N-Ph | t-bu | t-bu | H |
| 2-23-119 | N-Ph | N-Ph | methyl | methyl | H |
| 2-23-120 | N-Ph | N-Ph | b1 | b1 | H |
| 2-23-121 | N-Ph | N-Ph | b2 | b2 | H |
| 2-23-122 | N-Ph | N-Ph | b3 | b3 | H |
| 2-23-123 | N-Ph | N-Ph | H | H | H |
| 2-23-124 | N-Ph | N-Ph | t-bu | t-bu | H |
| 2-23-125 | N-Ph | N-Ph | methyl | methyl | H |
| 2-23-126 | N-Ph | N-Ph | b1 | b1 | H |
| 2-23-127 | N-Ph | N-Ph | b2 | b2 | H |
| 2-23-128 | N-Ph | N-Ph | b3 | b3 | H |
| 2-23-129 | N-Ph | N-Ph | H | H | H |
| 2-23-130 | N-Ph | N-Ph | t-bu | t-bu | H |
| 2-23-131 | N-Ph | N-Ph | methyl | methyl | H |
| 2-23-132 | N-Ph | N-Ph | b1 | b1 | H |
| 2-23-133 | N-Ph | N-Ph | b2 | b2 | H |
| 2-23-134 | N-Ph | N-Ph | b3 | b3 | H |
| 2-23-135 | N-Ph | N-Ph | H | H | H |
| 2-23-136 | N-Ph | N-Ph | t-bu | t-bu | H |
| 2-23-137 | N-Ph | N-Ph | methyl | methyl | H |
| 2-23-138 | N-Ph | N-Ph | b1 | b1 | H |
| 2-23-139 | N-Ph | N-Ph | b2 | b2 | H |
| 2-23-140 | N-Ph | N-Ph | b3 | b3 | H |
| 2-23-141 | N-Ph | N-Ph | H | H | H |
| 2-23-142 | N-Ph | N-Ph | t-bu | t-bu | H |
| 2-23-143 | N-Ph | N-Ph | methyl | methyl | H |
| 2-23-144 | N-Ph | N-Ph | b1 | b1 | H |
| 2-23-145 | N-Ph | N-Ph | b2 | b2 | H |
| 2-23-146 | N-Ph | N-Ph | b3 | b3 | H |
| 2-23-147 | N-Ph | N-Ph | H | H | H |
| 2-23-148 | N-Ph | N-Ph | t-bu | t-bu | H |
| 2-23-149 | N-Ph | N-Ph | methyl | methyl | H |
| 2-23-150 | N-Ph | N-Ph | b1 | b1 | H |
| 2-23-151 | N-Ph | N-Ph | b2 | b2 | H |
| 2-23-152 | N-Ph | N-Ph | b3 | b3 | H |
| 2-23-153 | N-Ph | N-Ph | H | H | H |
| 2-23-154 | N-Ph | N-Ph | t-bu | t-bu | H |
| 2-23-155 | N-Ph | N-Ph | methyl | methyl | H |
| 2-23-156 | N-Ph | N-Ph | b1 | b1 | H |
| 2-23-157 | N-Ph | N-Ph | b2 | b2 | H |
| 2-23-158 | N-Ph | N-Ph | b3 | b3 | H |
| 2-23-159 | N-Ph | N-Ph | H | H | H |
| 2-23-160 | N-Ph | N-Ph | t-bu | t-bu | H |
| 2-23-161 | N-Ph | N-Ph | methyl | methyl | H |
| 2-23-162 | N-Ph | N-Ph | b1 | b1 | H |
| 2-23-163 | N-Ph | N-Ph | b2 | b2 | H |
| 2-23-164 | N-Ph | N-Ph | b3 | b3 | H |
| 2-23-165 | N-Ph | N-Ph | H | H | H |
| 2-23-166 | N-Ph | N-Ph | t-bu | t-bu | H |
| 2-23-167 | N-Ph | N-Ph | methyl | methyl | H |
| 2-23-168 | N-Ph | N-Ph | b1 | b1 | H |
| 2-23-169 | N-Ph | N-Ph | b2 | b2 | H |
| 2-23-170 | N-Ph | N-Ph | b3 | b3 | H |
| 2-23-171 | N-Ph | N-Ph | H | H | H |
| 2-23-172 | N-Ph | N-Ph | t-bu | t-bu | H |
| 2-23-173 | N-Ph | N-Ph | methyl | methyl | H |
| 2-23-174 | N-Ph | N-Ph | b1 | b1 | H |
| 2-23-175 | N-Ph | N-Ph | b2 | b2 | H |
| 2-23-176 | N-Ph | N-Ph | b3 | b3 | H |
| 2-23-177 | N-Ph | N-Ph | H | H | H |
| 2-23-178 | N-Ph | N-Ph | t-bu | t-bu | H |
| 2-23-179 | N-Ph | N-Ph | methyl | methyl | H |
| 2-23-180 | N-Ph | N-Ph | b1 | b1 | H |
| 2-23-181 | N-Ph | N-Ph | b2 | b2 | H |
| 2-23-182 | N-Ph | N-Ph | b3 | b3 | H |
| 2-23-183 | N-Ph | N-Ph | H | H | H |
| 2-23-184 | N-Ph | N-Ph | t-bu | t-bu | H |
| 2-23-185 | N-Ph | N-Ph | methyl | methyl | H |
| 2-23-186 | N-Ph | N-Ph | b1 | b1 | H |
| 2-23-187 | N-Ph | N-Ph | b2 | b2 | H |
| 2-23-188 | N-Ph | N-Ph | b3 | b3 | H |
| 2-23-189 | N-Ph | N-Ph | H | H | H |
| 2-23-190 | N-Ph | N-Ph | t-bu | t-bu | H |
| 2-23-191 | N-Ph | N-Ph | methyl | methyl | H |
| 2-23-192 | N-Ph | N-Ph | b1 | b1 | H |
| 2-23-193 | N-Ph | N-Ph | b2 | b2 | H |
| 2-23-194 | N-Ph | N-Ph | b3 | b3 | H |
| 2-23-195 | N-Ph | N-Ph | H | H | H |
| 2-23-196 | N-Ph | N-Ph | t-bu | t-bu | H |
| 2-23-197 | N-Ph | N-Ph | methyl | methyl | H |
| 2-23-198 | N-Ph | N-Ph | b1 | b1 | H |
| 2-23-199 | N-Ph | N-Ph | b2 | b2 | H |
| 2-23-200 | N-Ph | N-Ph | b3 | b3 | H |
| 2-23-201 | N-Ph | N-Ph | H | H | a1 |
| 2-23-202 | N-Ph | N-Ph | t-bu | t-bu | a1 |
| 2-23-203 | N-Ph | N-Ph | methyl | methyl | a1 |
| 2-23-204 | N-Ph | N-Ph | b1 | b1 | a1 |
| 2-23-205 | N-Ph | N-Ph | b2 | b2 | a1 |
| 2-23-206 | N-Ph | N-Ph | b3 | b3 | a1 |
| 2-23-207 | N-Ph | N-Ph | H | H | a2 |
| 2-23-208 | N-Ph | N-Ph | t-bu | t-bu | a2 |
| 2-23-209 | N-Ph | N-Ph | methyl | methyl | a2 |
| 2-23-210 | N-Ph | N-Ph | b1 | b1 | a2 |
| 2-23-211 | N-Ph | N-Ph | b2 | b2 | a2 |
| 2-23-212 | N-Ph | N-Ph | b3 | b3 | a2 |
| 2-23-213 | N-Ph | N-Ph | H | H | a3 |
| 2-23-214 | N-Ph | N-Ph | t-bu | t-bu | a3 |
| 2-23-215 | N-Ph | N-Ph | methyl | methyl | a3 |
| 2-23-216 | N-Ph | N-Ph | b1 | b1 | a3 |
| 2-23-217 | N-Ph | N-Ph | b2 | b2 | a3 |
| 2-23-218 | N-Ph | N-Ph | b3 | b3 | a3 |
| 2-23-219 | N-Ph | N-Ph | H | H | b1 |
| 2-23-220 | N-Ph | N-Ph | t-bu | t-bu | b1 |
| 2-23-221 | N-Ph | N-Ph | methyl | methyl | b1 |
| 2-23-222 | N-Ph | N-Ph | b1 | b1 | b1 |
| 2-23-223 | N-Ph | N-Ph | b2 | b2 | b1 |
| 2-23-224 | N-Ph | N-Ph | b3 | b3 | b1 |
| 2-23-225 | N-Ph | N-Ph | H | H | b2 |
| 2-23-226 | N-Ph | N-Ph | t-bu | t-bu | b2 |
| 2-23-227 | N-Ph | N-Ph | methyl | methyl | b2 |
| 2-23-228 | N-Ph | N-Ph | b1 | b1 | b2 |
| 2-23-229 | N-Ph | N-Ph | b2 | b2 | b2 |
| 2-23-230 | N-Ph | N-Ph | b3 | b3 | b2 |
| 2-23-231 | N-Ph | N-Ph | H | H | b3 |
| 2-23-232 | N-Ph | N-Ph | t-bu | t-bu | b3 |
| 2-23-233 | N-Ph | N-Ph | methyl | methyl | b3 |
| 2-23-234 | N-Ph | N-Ph | b1 | b1 | b3 |
| 2-23-235 | N-Ph | N-Ph | b2 | b2 | b3 |
| 2-23-236 | N-Ph | N-Ph | b3 | b3 | b3 |
| 2-23-237 | N-Ph | N-Ph | H | H | b5 |
| 2-23-238 | N-Ph | N-Ph | t-bu | t-bu | b5 |
| 2-23-239 | N-Ph | N-Ph | methyl | methyl | b5 |
| 2-23-240 | N-Ph | N-Ph | b1 | b1 | b5 |
| 2-23-241 | N-Ph | N-Ph | b2 | b2 | b5 |
| 2-23-242 | N-Ph | N-Ph | b3 | b3 | b5 |
| 2-23-243 | N-Ph | N-Ph | H | H | b6 |
| 2-23-244 | N-Ph | N-Ph | t-bu | t-bu | b6 |
| 2-23-245 | N-Ph | N-Ph | methyl | methyl | b6 |
| 2-23-246 | N-Ph | N-Ph | b1 | b1 | b6 |
| 2-23-247 | N-Ph | N-Ph | b2 | b2 | b6 |
| 2-23-248 | N-Ph | N-Ph | b3 | b3 | b6 |
| 2-23-249 | N-Ph | N-Ph | H | H | b7 |
| 2-23-250 | N-Ph | N-Ph | t-bu | t-bu | b7 |
| 2-23-251 | N-Ph | N-Ph | methyl | methyl | b7 |
| 2-23-252 | N-Ph | N-Ph | b1 | b1 | b7 |
| 2-23-253 | N-Ph | N-Ph | b2 | b2 | b7 |
| 2-23-254 | N-Ph | N-Ph | b3 | b3 | b7 |
| 2-23-255 | N-tolyl | N-tolyl | H | H | H |
| 2-23-256 | N-tolyl | N-tolyl | t-bu | t-bu | H |
| 2-23-257 | N-tolyl | N-tolyl | methyl | methyl | H |
| 2-23-258 | N-tolyl | N-tolyl | t-bu | t-bu | t-bu |
| 2-23-259 | N-tolyl | N-tolyl | methyl | methyl | methyl |
| 2-23-260 | N-tolyl | N-tolyl | b1 | b1 | H |
| 2-23-261 | N-tolyl | N-tolyl | b2 | b2 | H |
| 2-23-262 | N-tolyl | N-tolyl | b3 | b3 | H |
| 2-23-263 | N-tolyl | N-tolyl | H | H | al |
| 2-23-264 | N-tolyl | N-tolyl | t-bu | t-bu | al |
| 2-23-265 | N-tolyl | N-tolyl | methyl | methyl | al |
| 2-23-266 | N-tolyl | N-tolyl | b1 | b1 | a1 |
| 2-23-267 | N-tolyl | N-tolyl | b2 | b2 | a1 |
| 2-23-268 | N-tolyl | N-tolyl | b3 | b3 | a1 |
| 2-23-269 | N-tolyl | N-tolyl | H | H | a2 |
| 2-23-270 | N-tolyl | N-tolyl | t-bu | t-bu | a2 |
| 2-23-271 | N-tolyl | N-tolyl | methyl | methyl | a2 |
| 2-23-272 | N-tolyl | N-tolyl | b1 | b1 | a2 |
| 2-23-273 | N-tolyl | N-tolyl | b2 | b2 | a2 |
| 2-23-274 | N-tolyl | N-tolyl | b3 | b3 | a2 |
| 2-23-275 | N-tolyl | N-tolyl | H | H | a3 |
| 2-23-276 | N-tolyl | N-tolyl | t-bu | t-bu | a3 |
| 2-23-277 | N-tolyl | N-tolyl | methyl | methyl | a3 |
| 2-23-278 | N-tolyl | N-tolyl | b1 | b1 | a3 |
| 2-23-279 | N-tolyl | N-tolyl | b2 | b2 | a3 |
| 2-23-280 | N-tolyl | N-tolyl | b3 | b3 | a3 |
| 2-23-281 | N-tolyl | N-tolyl | H | H | a4 |
| 2-23-282 | N-tolyl | N-tolyl | t-bu | t-bu | a4 |
| 2-23-283 | N-tolyl | N-tolyl | methyl | methyl | a4 |
| 2-23-284 | N-tolyl | N-tolyl | b1 | b1 | a4 |
| 2-23-285 | N-tolyl | N-tolyl | b2 | b2 | a4 |
| 2-23-286 | N-tolyl | N-tolyl | b3 | b3 | a4 |
| 2-23-287 | N-tolyl | N-tolyl | H | H | a5 |
| 2-23-288 | N-tolyl | N-tolyl | t-bu | t-bu | a5 |
| 2-23-289 | N-tolyl | N-tolyl | methyl | methyl | a5 |
| 2-23-290 | N-tolyl | N-tolyl | b1 | b1 | a5 |
| 2-23-291 | N-tolyl | N-tolyl | b2 | b2 | a5 |
| 2-23-292 | N-tolyl | N-tolyl | b3 | b3 | a5 |
| 2-23-293 | N-tolyl | N-tolyl | H | H | a6 |
| 2-23-294 | N-tolyl | N-tolyl | t-bu | t-bu | a6 |
| 2-23-295 | N-tolyl | N-tolyl | methyl | methyl | a6 |
| 2-23-296 | N-tolyl | N-tolyl | b1 | b1 | a6 |
| 2-23-297 | N-tolyl | N-tolyl | b2 | b2 | a6 |
| 2-23-298 | N-tolyl | N-tolyl | b3 | b3 | a6 |
| 2-23-299 | N-tolyl | N-tolyl | H | H | b1 |
| 2-23-300 | N-tolyl | N-tolyl | t-bu | t-bu | b1 |
| 2-23-301 | N-tolyl | N-tolyl | methyl | methyl | b1 |
| 2-23-302 | N-tolyl | N-tolyl | b1 | b1 | b1 |
| 2-23-303 | N-tolyl | N-tolyl | b2 | b2 | b1 |
| 2-23-304 | N-tolyl | N-tolyl | b3 | b3 | b1 |
| 2-23-305 | N-tolyl | N-tolyl | H | H | b2 |
| 2-23-306 | N-tolyl | N-tolyl | t-bu | t-bu | b2 |
| 2-23-307 | N-tolyl | N-tolyl | methyl | methyl | b2 |
| 2-23-308 | N-tolyl | N-tolyl | b1 | b1 | b2 |
| 2-23-309 | N-tolyl | N-tolyl | b2 | b2 | b2 |
| 2-23-310 | N-tolyl | N-tolyl | b3 | b3 | b2 |
| 2-23-311 | N-tolyl | N-tolyl | H | H | b3 |
| 2-23-312 | N-tolyl | N-tolyl | t-bu | t-bu | b3 |
| 2-23-313 | N-tolyl | N-tolyl | methyl | methyl | b3 |
| 2-23-314 | N-tolyl | N-tolyl | b1 | b1 | b3 |
| 2-23-315 | N-tolyl | N-tolyl | b2 | b2 | b3 |
| 2-23-316 | N-tolyl | N-tolyl | b3 | b3 | b3 |
| 2-23-317 | N-tolyl | N-tolyl | H | H | b4 |
| 2-23-318 | N-tolyl | N-tolyl | t-bu | t-bu | b4 |
| 2-23-319 | N-tolyl | N-tolyl | methyl | methyl | b4 |
| 2-23-320 | N-tolyl | N-tolyl | b1 | b1 | b4 |
| 2-23-321 | N-tolyl | N-tolyl | b2 | b2 | b4 |
| 2-23-322 | N-tolyl | N-tolyl | b3 | b3 | b4 |
| 2-23-323 | N-tolyl | N-tolyl | H | H | b5 |
| 2-23-324 | N-tolyl | N-tolyl | t-bu | t-bu | b5 |
| 2-23-325 | N-tolyl | N-tolyl | methyl | methyl | b5 |
| 2-23-326 | N-tolyl | N-tolyl | b1 | b1 | b5 |
| 2-23-327 | N-tolyl | N-tolyl | b2 | b2 | b5 |
| 2-23-328 | N-tolyl | N-tolyl | b3 | b3 | b5 |
| 2-23-329 | N-tolyl | N-tolyl | H | H | b6 |
| 2-23-330 | N-tolyl | N-tolyl | t-bu | t-bu | b6 |
| 2-23-331 | N-tolyl | N-tolyl | methyl | methyl | b6 |
| 2-23-332 | N-tolyl | N-tolyl | b1 | b1 | b6 |
| 2-23-333 | N-tolyl | N-tolyl | b2 | b2 | b6 |
| 2-23-334 | N-tolyl | N-tolyl | b3 | b3 | b6 |
| 2-23-335 | N-tolyl | N-tolyl | H | H | b7 |
| 2-23-336 | N-tolyl | N-tolyl | t-bu | t-bu | b7 |
| 2-23-337 | N-tolyl | N-tolyl | methyl | methyl | b7 |
| 2-23-338 | N-tolyl | N-tolyl | b1 | b1 | b7 |
| 2-23-339 | N-tolyl | N-tolyl | b2 | b2 | b7 |
| 2-23-340 | N-tolyl | N-tolyl | b3 | b3 | b7 |
| 2-23-341 | N-tolyl | N-tolyl | H | H | b8 |
| 2-23-342 | N-tolyl | N-tolyl | t-bu | t-bu | b8 |
| 2-23-343 | N-tolyl | N-tolyl | methyl | methyl | b8 |
| 2-23-344 | N-tolyl | N-tolyl | b1 | b1 | b8 |
| 2-23-345 | N-tolyl | N-tolyl | b2 | b2 | b8 |
| 2-23-346 | N-tolyl | N-tolyl | b3 | b3 | b8 |
| 2-23-347 | N-tolyl | N-tolyl | H | H | b9 |
| 2-23-348 | N-tolyl | N-tolyl | t-bu | t-bu | b9 |
| 2-23-349 | N-tolyl | N-tolyl | methyl | methyl | b9 |
| 2-23-350 | N-tolyl | N-tolyl | b1 | b1 | b9 |
| 2-23-351 | N-tolyl | N-tolyl | b2 | b2 | b9 |
| 2-23-352 | N-tolyl | N-tolyl | b3 | b3 | b9 |
| 2-23-353 | N-tolyl | N-tolyl | H | H | b10 |
| 2-23-354 | N-tolyl | N-tolyl | t-bu | t-bu | b10 |
| 2-23-355 | N-tolyl | N-tolyl | methyl | methyl | b10 |
| 2-23-356 | N-tolyl | N-tolyl | b1 | b1 | b10 |
| 2-23-357 | N-tolyl | N-tolyl | b2 | b2 | b10 |
| 2-23-358 | N-tolyl | N-tolyl | b3 | b3 | b10 |
| 2-23-359 | N-tolyl | N-tolyl | H | H | H |
| 2-23-360 | N-tolyl | N-tolyl | t-bu | t-bu | H |
| 2-23-361 | N-tolyl | N-tolyl | methyl | methyl | H |
| 2-23-362 | N-tolyl | N-tolyl | b1 | b1 | H |
| 2-23-363 | N-tolyl | N-tolyl | b2 | b2 | H |
| 2-23-364 | N-tolyl | N-tolyl | b3 | b3 | H |
| 2-23-365 | N-tolyl | N-tolyl | H | H | H |
| 2-23-366 | N-tolyl | N-tolyl | t-bu | t-bu | H |
| 2-23-367 | N-tolyl | N-tolyl | methyl | methyl | H |
| 2-23-368 | N-tolyl | N-tolyl | b1 | b1 | H |
| 2-23-369 | N-tolyl | N-tolyl | b2 | b2 | H |
| 2-23-370 | N-tolyl | N-tolyl | b3 | b3 | H |
| 2-23-371 | N-tolyl | N-tolyl | H | H | H |
| 2-23-372 | N-tolyl | N-tolyl | t-bu | t-bu | H |
| 2-23-373 | N-tolyl | N-tolyl | methyl | methyl | H |
| 2-23-374 | N-tolyl | N-tolyl | b1 | b1 | H |
| 2-23-375 | N-tolyl | N-tolyl | b2 | b2 | H |
| 2-23-376 | N-tolyl | N-tolyl | b3 | b3 | H |
| 2-23-377 | N-tolyl | N-tolyl | H | H | H |
| 2-23-378 | N-tolyl | N-tolyl | t-bu | t-bu | H |
| 2-23-379 | N-tolyl | N-tolyl | methyl | methyl | H |
| 2-23-380 | N-tolyl | N-tolyl | b1 | b1 | H |
| 2-23-381 | N-tolyl | N-tolyl | b2 | b2 | H |
| 2-23-382 | N-tolyl | N-tolyl | b3 | b3 | H |
| 2-23-383 | N-tolyl | N-tolyl | H | H | H |
| 2-23-384 | N-tolyl | N-tolyl | t-bu | t-bu | H |
| 2-23-385 | N-tolyl | N-tolyl | methyl | methyl | H |
| 2-23-386 | N-tolyl | N-tolyl | b1 | b1 | H |
| 2-23-387 | N-tolyl | N-tolyl | b2 | b2 | H |
| 2-23-388 | N-tolyl | N-tolyl | b3 | b3 | H |
| 2-23-389 | N-tolyl | N-tolyl | H | H | H |
| 2-23-390 | N-tolyl | N-tolyl | t-bu | t-bu | H |
| 2-23-391 | N-tolyl | N-tolyl | methyl | methyl | H |
| 2-23-392 | N-tolyl | N-tolyl | b1 | b1 | H |
| 2-23-393 | N-tolyl | N-tolyl | b2 | b2 | H |
| 2-23-394 | N-tolyl | N-tolyl | b3 | b3 | H |
| 2-23-395 | N-tolyl | N-tolyl | H | H | H |
| 2-23-396 | N-tolyl | N-tolyl | t-bu | t-bu | H |
| 2-23-397 | N-tolyl | N-tolyl | methyl | methyl | H |
| 2-23-398 | N-tolyl | N-tolyl | b1 | b1 | H |
| 2-23-399 | N-tolyl | N-tolyl | b2 | b2 | H |
| 2-23-400 | N-tolyl | N-tolyl | b3 | b3 | H |
| 2-23-401 | N-tolyl | N-tolyl | H | H | H |
| 2-23-402 | N-tolyl | N-tolyl | t-bu | t-bu | H |
| 2-23-403 | N-tolyl | N-tolyl | methyl | methyl | H |
| 2-23-404 | N-tolyl | N-tolyl | b1 | b1 | H |
| 2-23-405 | N-tolyl | N-tolyl | b2 | b2 | H |
| 2-23-406 | N-tolyl | N-tolyl | b3 | b3 | H |
| 2-23-407 | N-tolyl | N-tolyl | H | H | H |
| 2-23-408 | N-tolyl | N-tolyl | t-bu | t-bu | H |
| 2-23-409 | N-tolyl | N-tolyl | methyl | methyl | H |
| 2-23-410 | N-tolyl | N-tolyl | b1 | b1 | H |
| 2-23-411 | N-tolyl | N-tolyl | b2 | b2 | H |
| 2-23-412 | N-tolyl | N-tolyl | b3 | b3 | H |
| 2-23-413 | N-tolyl | N-tolyl | H | H | H |
| 2-23-414 | N-tolyl | N-tolyl | t-bu | t-bu | H |
| 2-23-415 | N-tolyl | N-tolyl | methyl | methyl | H |
| 2-23-416 | N-tolyl | N-tolyl | b1 | b1 | H |
| 2-23-417 | N-tolyl | N-tolyl | b2 | b2 | H |
| 2-23-418 | N-tolyl | N-tolyl | b3 | b3 | H |
| 2-23-419 | N-tolyl | N-tolyl | H | H | H |
| 2-23-420 | N-tolyl | N-tolyl | t-bu | t-bu | H |
| 2-23-421 | N-tolyl | N-tolyl | methyl | methyl | H |
| 2-23-422 | N-tolyl | N-tolyl | b1 | b1 | H |
| 2-23-423 | N-tolyl | N-tolyl | b2 | b2 | H |
| 2-23-424 | N-tolyl | N-tolyl | b3 | b3 | H |
| 2-23-425 | N-tolyl | N-tolyl | H | H | H |
| 2-23-426 | N-tolyl | N-tolyl | t-bu | t-bu | H |
| 2-23-427 | N-tolyl | N-tolyl | methyl | methyl | H |
| 2-23-428 | N-tolyl | N-tolyl | b1 | b1 | H |
| 2-23-429 | N-tolyl | N-tolyl | b2 | b2 | H |
| 2-23-430 | N-tolyl | N-tolyl | b3 | b3 | H |
| 2-23-431 | N-tolyl | N-tolyl | H | H | H |
| 2-23-432 | N-tolyl | N-tolyl | t-bu | t-bu | H |
| 2-23-433 | N-tolyl | N-tolyl | methyl | methyl | H |
| 2-23-434 | N-tolyl | N-tolyl | b1 | b1 | H |
| 2-23-435 | N-tolyl | N-tolyl | b2 | b2 | H |
| 2-23-436 | N-tolyl | N-tolyl | b3 | b3 | H |
| 2-23-437 | N-tolyl | N-tolyl | H | H | H |
| 2-23-438 | N-tolyl | N-tolyl | t-bu | t-bu | H |
| 2-23-439 | N-tolyl | N-tolyl | methyl | methyl | H |
| 2-23-440 | N-tolyl | N-tolyl | b1 | b1 | H |
| 2-23-441 | N-tolyl | N-tolyl | b2 | b2 | H |
| 2-23-442 | N-tolyl | N-tolyl | b3 | b3 | H |
| 2-23-443 | N-tolyl | N-tolyl | H | H | H |
| 2-23-444 | N-tolyl | N-tolyl | t-bu | t-bu | H |
| 2-23-445 | N-tolyl | N-tolyl | methyl | methyl | H |
| 2-23-446 | N-tolyl | N-tolyl | b1 | b1 | H |
| 2-23-447 | N-tolyl | N-tolyl | b2 | b2 | H |
| 2-23-448 | N-tolyl | N-tolyl | b3 | b3 | H |
| 2-23-449 | N-tolyl | N-tolyl | H | H | H |
| 2-23-450 | N-tolyl | N-tolyl | t-bu | t-bu | H |
| 2-23-451 | N-tolyl | N-tolyl | methyl | methyl | H |
| 2-23-452 | N-tolyl | N-tolyl | b1 | b1 | H |
| 2-23-453 | N-tolyl | N-tolyl | b2 | b2 | H |
| 2-23-454 | N-tolyl | N-tolyl | b3 | b3 | H |
| 2-23-455 | N-tolyl | N-tolyl | H | H | a1 |
| 2-23-456 | N-tolyl | N-tolyl | t-bu | t-bu | a1 |
| 2-23-457 | N-tolyl | N-tolyl | methyl | methyl | a1 |
| 2-23-458 | N-tolyl | N-tolyl | b1 | b1 | a1 |
| 2-23-459 | N-tolyl | N-tolyl | b2 | b2 | a1 |
| 2-23-460 | N-tolyl | N-tolyl | b3 | b3 | a1 |
| 2-23-461 | N-tolyl | N-tolyl | H | H | a2 |
| 2-23-462 | N-tolyl | N-tolyl | t-bu | t-bu | a2 |
| 2-23-463 | N-tolyl | N-tolyl | methyl | methyl | a2 |
| 2-23-464 | N-tolyl | N-tolyl | b1 | b1 | a2 |
| 2-23-465 | N-tolyl | N-tolyl | b2 | b2 | a2 |
| 2-23-466 | N-tolyl | N-tolyl | b3 | b3 | a2 |
| 2-23-467 | N-tolyl | N-tolyl | H | H | a3 |
| 2-23-468 | N-tolyl | N-tolyl | t-bu | t-bu | a3 |
| 2-23-469 | N-tolyl | N-tolyl | methyl | methyl | a3 |
| 2-23-470 | N-tolyl | N-tolyl | b1 | b1 | a3 |
| 2-23-471 | N-tolyl | N-tolyl | b2 | b2 | a3 |
| 2-23-472 | N-tolyl | N-tolyl | b3 | b3 | a3 |
| 2-23-473 | N-tolyl | N-tolyl | H | H | b1 |
| 2-23-474 | N-tolyl | N-tolyl | t-bu | t-bu | b1 |
| 2-23-475 | N-tolyl | N-tolyl | methyl | methyl | b1 |
| 2-23-476 | N-tolyl | N-tolyl | b1 | b1 | b1 |
| 2-23-477 | N-tolyl | N-tolyl | b2 | b2 | b1 |
| 2-23-478 | N-tolyl | N-tolyl | b3 | b3 | b1 |
| 2-23-479 | N-tolyl | N-tolyl | H | H | b2 |
| 2-23-480 | N-tolyl | N-tolyl | t-bu | t-bu | b2 |
| 2-23-481 | N-tolyl | N-tolyl | methyl | methyl | b2 |
| 2-23-482 | N-tolyl | N-tolyl | b1 | b1 | b2 |
| 2-23-483 | N-tolyl | N-tolyl | b2 | b2 | b2 |
| 2-23-484 | N-tolyl | N-tolyl | b3 | b3 | b2 |
| 2-23-485 | N-tolyl | N-tolyl | H | H | b3 |
| 2-23-486 | N-tolyl | N-tolyl | t-bu | t-bu | b3 |
| 2-23-487 | N-tolyl | N-tolyl | methyl | methyl | b3 |
| 2-23-488 | N-tolyl | N-tolyl | b1 | b1 | b3 |
| 2-23-489 | N-tolyl | N-tolyl | b2 | b2 | b3 |
| 2-23-490 | N-tolyl | N-tolyl | b3 | b3 | b3 |
| 2-23-491 | N-tolyl | N-tolyl | H | H | b5 |
| 2-23-492 | N-tolyl | N-tolyl | t-bu | t-bu | b5 |
| 2-23-493 | N-tolyl | N-tolyl | methyl | methyl | b5 |
| 2-23-494 | N-tolyl | N-tolyl | b1 | b1 | b5 |
| 2-23-495 | N-tolyl | N-tolyl | b2 | b2 | b5 |
| 2-23-496 | N-tolyl | N-tolyl | b3 | b3 | b5 |
| 2-23-497 | N-tolyl | N-tolyl | H | H | b6 |
| 2-23-498 | N-tolyl | N-tolyl | t-bu | t-bu | b6 |
| 2-23-499 | N-tolyl | N-tolyl | methyl | methyl | b6 |
| 2-23-500 | N-tolyl | N-tolyl | b1 | b1 | b6 |
| 2-23-501 | N-tolyl | N-tolyl | b2 | b2 | b6 |
| 2-23-502 | N-tolyl | N-tolyl | b3 | b3 | b6 |
| 2-23-503 | N-tolyl | N-tolyl | H | H | b7 |
| 2-23-504 | N-tolyl | N-tolyl | t-bu | t-bu | b7 |
| 2-23-505 | N-tolyl | N-tolyl | methyl | methyl | b7 |
| 2-23-506 | N-tolyl | N-tolyl | b1 | b1 | b7 |
| 2-23-507 | N-tolyl | N-tolyl | b2 | b2 | b7 |
| 2-23-508 | N-tolyl | N-tolyl | b3 | b3 | b7 |
| 2-23-509 | N-Biph | N-Biph | H | H | H |
| 2-23-510 | N-Biph | N-Biph | t-bu | t-bu | H |
| 2-23-511 | N-Biph | N-Biph | methyl | methyl | H |
| 2-23-512 | N-Biph | N-Biph | t-bu | t-bu | t-bu |
| 2-23-513 | N-Biph | N-Biph | methyl | methyl | methyl |
| 2-23-514 | N-Biph | N-Biph | b1 | b1 | H |
| 2-23-515 | N-Biph | N-Biph | b2 | b2 | H |
| 2-23-516 | N-Biph | N-Biph | b3 | b3 | H |
| 2-23-517 | N-Biph | N-Biph | H | H | a1 |
| 2-23-518 | N-Biph | N-Biph | t-bu | t-bu | a1 |
| 2-23-519 | N-Biph | N-Biph | methyl | methyl | a1 |
| 2-23-520 | N-Biph | N-Biph | b1 | b1 | a1 |
| 2-23-521 | N-Biph | N-Biph | b2 | b2 | a1 |
| 2-23-522 | N-Biph | N-Biph | b3 | b3 | a1 |
| 2-23-523 | N-Biph | N-Biph | H | H | a2 |
| 2-23-524 | N-Biph | N-Biph | t-bu | t-bu | a2 |
| 2-23-525 | N-Biph | N-Biph | methyl | methyl | a2 |
| 2-23-526 | N-Biph | N-Biph | b1 | b1 | a2 |
| 2-23-527 | N-Biph | N-Biph | b2 | b2 | a2 |
| 2-23-528 | N-Biph | N-Biph | b3 | b3 | a2 |
| 2-23-529 | N-Biph | N-Biph | H | H | a3 |
| 2-23-530 | N-Biph | N-Biph | t-bu | t-bu | a3 |
| 2-23-531 | N-Biph | N-Biph | methyl | methyl | a3 |
| 2-23-532 | N-Biph | N-Biph | b1 | b1 | a3 |
| 2-23-533 | N-Biph | N-Biph | b2 | b2 | a3 |
| 2-23-534 | N-Biph | N-Biph | b3 | b3 | a3 |
| 2-23-535 | N-Biph | N-Biph | H | H | a4 |
| 2-23-536 | N-Biph | N-Biph | t-bu | t-bu | a4 |
| 2-23-537 | N-Biph | N-Biph | methyl | methyl | a4 |
| 2-23-538 | N-Biph | N-Biph | b1 | b1 | a4 |
| 2-23-539 | N-Biph | N-Biph | b2 | b2 | a4 |
| 2-23-540 | N-Biph | N-Biph | b3 | b3 | a4 |
| 2-23-541 | N-Biph | N-Biph | H | H | a5 |
| 2-23-542 | N-Biph | N-Biph | t-bu | t-bu | a5 |
| 2-23-543 | N-Biph | N-Biph | methyl | methyl | a5 |
| 2-23-544 | N-Biph | N-Biph | b1 | b1 | a5 |
| 2-23-545 | N-Biph | N-Biph | b2 | b2 | a5 |
| 2-23-546 | N-Biph | N-Biph | b3 | b3 | a5 |
| 2-23-547 | N-Biph | N-Biph | H | H | a6 |
| 2-23-548 | N-Biph | N-Biph | t-bu | t-bu | a6 |
| 2-23-549 | N-Biph | N-Biph | methyl | methyl | a6 |
| 2-23-550 | N-Biph | N-Biph | b1 | b1 | a6 |
| 2-23-551 | N-Biph | N-Biph | b2 | b2 | a6 |
| 2-23-552 | N-Biph | N-Biph | b3 | b3 | a6 |
| 2-23-553 | N-Biph | N-Biph | H | H | b1 |
| 2-23-554 | N-Biph | N-Biph | t-bu | t-bu | b1 |
| 2-23-555 | N-Biph | N-Biph | methyl | methyl | b1 |
| 2-23-556 | N-Biph | N-Biph | b1 | b1 | b1 |
| 2-23-557 | N-Biph | N-Biph | b2 | b2 | b1 |
| 2-23-558 | N-Biph | N-Biph | b3 | b3 | b1 |
| 2-23-559 | N-Biph | N-Biph | H | H | b2 |
| 2-23-560 | N-Biph | N-Biph | t-bu | t-bu | b2 |
| 2-23-561 | N-Biph | N-Biph | methyl | methyl | b2 |
| 2-23-562 | N-Biph | N-Biph | b1 | b1 | b2 |
| 2-23-563 | N-Biph | N-Biph | b2 | b2 | b2 |
| 2-23-564 | N-Biph | N-Biph | b3 | b3 | b2 |
| 2-23-565 | N-Biph | N-Biph | H | H | b3 |
| 2-23-566 | N-Biph | N-Biph | t-bu | t-bu | b3 |
| 2-23-567 | N-Biph | N-Biph | methyl | methyl | b3 |
| 2-23-568 | N-Biph | N-Biph | b1 | b1 | b3 |
| 2-23-569 | N-Biph | N-Biph | b2 | b2 | b3 |
| 2-23-570 | N-Biph | N-Biph | b3 | b3 | b3 |
| 2-23-571 | N-Biph | N-Biph | H | H | b4 |
| 2-23-572 | N-Biph | N-Biph | t-bu | t-bu | b4 |
| 2-23-573 | N-Biph | N-Biph | methyl | methyl | b4 |
| 2-23-574 | N-Biph | N-Biph | b1 | b1 | b4 |
| 2-23-575 | N-Biph | N-Biph | b2 | b2 | b4 |
| 2-23-576 | N-Biph | N-Biph | b3 | b3 | b4 |
| 2-23-577 | N-Biph | N-Biph | H | H | b5 |
| 2-23-578 | N-Biph | N-Biph | t-bu | t-bu | b5 |
| 2-23-579 | N-Biph | N-Biph | methyl | methyl | b5 |
| 2-23-580 | N-Biph | N-Biph | b1 | b1 | b5 |
| 2-23-581 | N-Biph | N-Biph | b2 | b2 | b5 |
| 2-23-582 | N-Biph | N-Biph | b3 | b3 | b5 |
| 2-23-583 | N-Biph | N-Biph | H | H | b6 |
| 2-23-584 | N-Biph | N-Biph | t-bu | t-bu | b6 |
| 2-23-585 | N-Biph | N-Biph | methyl | methyl | b6 |
| 2-23-586 | N-Biph | N-Biph | b1 | b1 | b6 |
| 2-23-587 | N-Biph | N-Biph | b2 | b2 | b6 |
| 2-23-588 | N-Biph | N-Biph | b3 | b3 | b6 |
| 2-23-589 | N-Biph | N-Biph | H | H | b7 |
| 2-23-590 | N-Biph | N-Biph | t-bu | t-bu | b7 |
| 2-23-591 | N-Biph | N-Biph | methyl | methyl | b7 |
| 2-23-592 | N-Biph | N-Biph | b1 | b1 | b7 |
| 2-23-593 | N-Biph | N-Biph | b2 | b2 | b7 |
| 2-23-594 | N-Biph | N-Biph | b3 | b3 | b7 |
| 2-23-595 | N-Biph | N-Biph | H | H | b8 |
| 2-23-596 | N-Biph | N-Biph | t-bu | t-bu | b8 |
| 2-23-597 | N-Biph | N-Biph | methyl | methyl | b8 |
| 2-23-598 | N-Biph | N-Biph | b1 | b1 | b8 |
| 2-23-599 | N-Biph | N-Biph | b2 | b2 | b8 |
| 2-23-600 | N-Biph | N-Biph | b3 | b3 | b8 |
| 2-23-601 | N-Biph | N-Biph | H | H | b9 |
| 2-23-602 | N-Biph | N-Biph | t-bu | t-bu | b9 |
| 2-23-603 | N-Biph | N-Biph | methyl | methyl | b9 |
| 2-23-604 | N-Biph | N-Biph | b1 | b1 | b9 |
| 2-23-605 | N-Biph | N-Biph | b2 | b2 | b9 |
| 2-23-606 | N-Biph | N-Biph | b3 | b3 | b9 |
| 2-23-607 | N-Biph | N-Biph | H | H | b10 |
| 2-23-608 | N-Biph | N-Biph | t-bu | t-bu | b10 |
| 2-23-609 | N-Biph | N-Biph | methyl | methyl | b10 |
| 2-23-610 | N-Biph | N-Biph | b1 | b1 | b10 |
| 2-23-611 | N-Biph | N-Biph | b2 | b2 | b10 |
| 2-23-612 | N-Biph | N-Biph | b3 | b3 | b10 |
| 2-23-613 | N-Biph | N-Biph | H | H | H |
| 2-23-614 | N-Biph | N-Biph | t-bu | t-bu | H |
| 2-23-615 | N-Biph | N-Biph | methyl | methyl | H |
| 2-23-616 | N-Biph | N-Biph | b1 | b1 | H |
| 2-23-617 | N-Biph | N-Biph | b2 | b2 | H |
| 2-23-618 | N-Biph | N-Biph | b3 | b3 | H |
| 2-23-619 | N-Biph | N-Biph | H | H | H |
| 2-23-620 | N-Biph | N-Biph | t-bu | t-bu | H |
| 2-23-621 | N-Biph | N-Biph | methyl | methyl | H |
| 2-23-622 | N-Biph | N-Biph | b1 | b1 | H |
| 2-23-623 | N-Biph | N-Biph | b2 | b2 | H |
| 2-23-624 | N-Biph | N-Biph | b3 | b3 | H |
| 2-23-625 | N-Biph | N-Biph | H | H | H |
| 2-23-626 | N-Biph | N-Biph | t-bu | t-bu | H |
| 2-23-627 | N-Biph | N-Biph | methyl | methyl | H |
| 2-23-628 | N-Biph | N-Biph | b1 | b1 | H |
| 2-23-629 | N-Biph | N-Biph | b2 | b2 | H |
| 2-23-630 | N-Biph | N-Biph | b3 | b3 | H |
| 2-23-631 | N-Biph | N-Biph | H | H | H |
| 2-23-632 | N-Biph | N-Biph | t-bu | t-bu | H |
| 2-23-633 | N-Biph | N-Biph | methyl | methyl | H |
| 2-23-634 | N-Biph | N-Biph | b1 | b1 | H |
| 2-23-635 | N-Biph | N-Biph | b2 | b2 | H |
| 2-23-636 | N-Biph | N-Biph | b3 | b3 | H |
| 2-23-637 | N-Biph | N-Biph | H | H | H |
| 2-23-638 | N-Biph | N-Biph | t-bu | t-bu | H |
| 2-23-639 | N-Biph | N-Biph | methyl | methyl | H |
| 2-23-640 | N-Biph | N-Biph | b1 | b1 | H |
| 2-23-641 | N-Biph | N-Biph | b2 | b2 | H |
| 2-23-642 | N-Biph | N-Biph | b3 | b3 | H |
| 2-23-643 | N-Biph | N-Biph | H | H | H |
| 2-23-644 | N-Biph | N-Biph | t-bu | t-bu | H |
| 2-23-645 | N-Biph | N-Biph | methyl | methyl | H |
| 2-23-646 | N-Biph | N-Biph | b1 | b1 | H |
| 2-23-647 | N-Biph | N-Biph | b2 | b2 | H |
| 2-23-648 | N-Biph | N-Biph | b3 | b3 | H |
| 2-23-649 | N-Biph | N-Biph | H | H | H |
| 2-23-650 | N-Biph | N-Biph | t-bu | t-bu | H |
| 2-23-651 | N-Biph | N-Biph | methyl | methyl | H |
| 2-23-652 | N-Biph | N-Biph | b1 | b1 | H |
| 2-23-653 | N-Biph | N-Biph | b2 | b2 | H |
| 2-23-654 | N-Biph | N-Biph | b3 | b3 | H |
| 2-23-655 | N-Biph | N-Biph | H | H | H |
| 2-23-656 | N-Biph | N-Biph | t-bu | t-bu | H |
| 2-23-657 | N-Biph | N-Biph | methyl | methyl | H |
| 2-23-658 | N-Biph | N-Biph | b1 | b1 | H |
| 2-23-659 | N-Biph | N-Biph | b2 | b2 | H |
| 2-23-660 | N-Biph | N-Biph | b3 | b3 | H |
| 2-23-661 | N-Biph | N-Biph | H | H | H |
| 2-23-662 | N-Biph | N-Biph | t-bu | t-bu | H |
| 2-23-663 | N-Biph | N-Biph | methyl | methyl | H |
| 2-23-664 | N-Biph | N-Biph | b1 | b1 | H |
| 2-23-665 | N-Biph | N-Biph | b2 | b2 | H |
| 2-23-666 | N-Biph | N-Biph | b3 | b3 | H |
| 2-23-667 | N-Biph | N-Biph | H | H | H |
| 2-23-668 | N-Biph | N-Biph | t-bu | t-bu | H |
| 2-23-669 | N-Biph | N-Biph | methyl | methyl | H |
| 2-23-670 | N-Biph | N-Biph | b1 | b1 | H |
| 2-23-671 | N-Biph | N-Biph | b2 | b2 | H |
| 2-23-672 | N-Biph | N-Biph | b3 | b3 | H |
| 2-23-673 | N-Biph | N-Biph | H | H | H |
| 2-23-674 | N-Biph | N-Biph | t-bu | t-bu | H |
| 2-23-675 | N-Biph | N-Biph | methyl | methyl | H |
| 2-23-676 | N-Biph | N-Biph | b1 | b1 | H |
| 2-23-677 | N-Biph | N-Biph | b2 | b2 | H |
| 2-23-678 | N-Biph | N-Biph | b3 | b3 | H |
| 2-23-679 | N-Biph | N-Biph | H | H | H |
| 2-23-680 | N-Biph | N-Biph | t-bu | t-bu | H |
| 2-23-681 | N-Biph | N-Biph | methyl | methyl | H |
| 2-23-682 | N-Biph | N-Biph | b1 | b1 | H |
| 2-23-683 | N-Biph | N-Biph | b2 | b2 | H |
| 2-23-684 | N-Biph | N-Biph | b3 | b3 | H |
| 2-23-685 | N-Biph | N-Biph | H | H | H |
| 2-23-686 | N-Biph | N-Biph | t-bu | t-bu | H |
| 2-23-687 | N-Biph | N-Biph | methyl | methyl | H |
| 2-23-688 | N-Biph | N-Biph | b1 | b1 | H |
| 2-23-689 | N-Biph | N-Biph | b2 | b2 | H |
| 2-23-690 | N-Biph | N-Biph | b3 | b3 | H |
| 2-23-691 | N-Biph | N-Biph | H | H | H |
| 2-23-692 | N-Biph | N-Biph | t-bu | t-bu | H |
| 2-23-693 | N-Biph | N-Biph | methyl | methyl | H |
| 2-23-694 | N-Biph | N-Biph | b1 | b1 | H |
| 2-23-695 | N-Biph | N-Biph | b2 | b2 | H |
| 2-23-696 | N-Biph | N-Biph | b3 | b3 | H |
| 2-23-697 | N-Biph | N-Biph | H | H | H |
| 2-23-698 | N-Biph | N-Biph | t-bu | t-bu | H |
| 2-23-699 | N-Biph | N-Biph | methyl | methyl | H |
| 2-23-700 | N-Biph | N-Biph | b1 | b1 | H |
| 2-23-701 | N-Biph | N-Biph | b2 | b2 | H |
| 2-23-702 | N-Biph | N-Biph | b3 | b3 | H |
| 2-23-703 | N-Biph | N-Biph | H | H | H |
| 2-23-704 | N-Biph | N-Biph | t-bu | t-bu | H |
| 2-23-705 | N-Biph | N-Biph | methyl | methyl | H |
| 2-23-706 | N-Biph | N-Biph | b1 | b1 | H |
| 2-23-707 | N-Biph | N-Biph | b2 | b2 | H |
| 2-23-708 | N-Biph | N-Biph | b3 | b3 | H |
| 2-23-709 | N-Biph | N-Biph | H | H | a1 |
| 2-23-710 | N-Biph | N-Biph | t-bu | t-bu | a1 |
| 2-23-711 | N-Biph | N-Biph | methyl | methyl | a1 |
| 2-23-712 | N-Biph | N-Biph | b1 | b1 | a1 |
| 2-23-713 | N-Biph | N-Biph | b2 | b2 | a1 |
| 2-23-714 | N-Biph | N-Biph | b3 | b3 | a1 |
| 2-23-715 | N-Biph | N-Biph | H | H | a2 |
| 2-23-716 | N-Biph | N-Biph | t-bu | t-bu | a2 |
| 2-23-717 | N-Biph | N-Biph | methyl | methyl | a2 |
| 2-23-718 | N-Biph | N-Biph | b1 | b1 | a2 |
| 2-23-719 | N-Biph | N-Biph | b2 | b2 | a2 |
| 2-23-720 | N-Biph | N-Biph | b3 | b3 | a2 |
| 2-23-721 | N-Biph | N-Biph | H | H | a3 |
| 2-23-722 | N-Biph | N-Biph | t-bu | t-bu | a3 |
| 2-23-723 | N-Biph | N-Biph | methyl | methyl | a3 |
| 2-23-724 | N-Biph | N-Biph | b1 | b1 | a3 |
| 2-23-725 | N-Biph | N-Biph | b2 | b2 | a3 |
| 2-23-726 | N-Biph | N-Biph | b3 | b3 | a3 |
| 2-23-727 | N-Biph | N-Biph | H | H | b1 |
| 2-23-728 | N-Biph | N-Biph | t-bu | t-bu | b1 |
| 2-23-729 | N-Biph | N-Biph | methyl | methyl | b1 |
| 2-23-730 | N-Biph | N-Biph | b1 | b1 | b1 |
| 2-23-731 | N-Biph | N-Biph | b2 | b2 | b1 |
| 2-23-732 | N-Biph | N-Biph | b3 | b3 | b1 |
| 2-23-733 | N-Biph | N-Biph | H | H | b2 |
| 2-23-734 | N-Biph | N-Biph | t-bu | t-bu | b2 |
| 2-23-735 | N-Biph | N-Biph | methyl | methyl | b2 |
| 2-23-736 | N-Biph | N-Biph | b1 | b1 | b2 |
| 2-23-737 | N-Biph | N-Biph | b2 | b2 | b2 |
| 2-23-738 | N-Biph | N-Biph | b3 | b3 | b2 |
| 2-23-739 | N-Biph | N-Biph | H | H | b3 |
| 2-23-740 | N-Biph | N-Biph | t-bu | t-bu | b3 |
| 2-23-741 | N-Biph | N-Biph | methyl | methyl | b3 |
| 2-23-742 | N-Biph | N-Biph | b1 | b1 | b3 |
| 2-23-743 | N-Biph | N-Biph | b2 | b2 | b3 |
| 2-23-744 | N-Biph | N-Biph | b3 | b3 | b3 |
| 2-23-745 | N-Biph | N-Biph | H | H | b5 |
| 2-23-746 | N-Biph | N-Biph | t-bu | t-bu | b5 |
| 2-23-747 | N-Biph | N-Biph | methyl | methyl | b5 |
| 2-23-748 | N-Biph | N-Biph | b1 | b1 | b5 |
| 2-23-749 | N-Biph | N-Biph | b2 | b2 | b5 |
| 2-23-750 | N-Biph | N-Biph | b3 | b3 | b5 |
| 2-23-751 | N-Biph | N-Biph | H | H | b6 |
| 2-23-752 | N-Biph | N-Biph | t-bu | t-bu | b6 |
| 2-23-753 | N-Biph | N-Biph | methyl | methyl | b6 |
| 2-23-754 | N-Biph | N-Biph | b1 | b1 | b6 |
| 2-23-755 | N-Biph | N-Biph | b2 | b2 | b6 |
| 2-23-756 | N-Biph | N-Biph | b3 | b3 | b6 |
| 2-23-757 | N-Biph | N-Biph | H | H | b7 |
| 2-23-758 | N-Biph | N-Biph | t-bu | t-bu | b7 |
| 2-23-759 | N-Biph | N-Biph | methyl | methyl | b7 |
| 2-23-760 | N-Biph | N-Biph | b1 | b1 | b7 |
| 2-23-761 | N-Biph | N-Biph | b2 | b2 | b7 |
| 2-23-762 | N-Biph | N-Biph | b3 | b3 | b7 |
| 2-23-763 | N-Ph | O | H | H | H |
| 2-23-764 | N-Ph | O | t-bu | t-bu | H |
| 2-23-765 | N-Ph | O | methyl | methyl | H |
| 2-23-766 | N-Ph | O | t-bu | t-bu | t-bu |
| 2-23-767 | N-Ph | O | methyl | methyl | methyl |
| 2-23-768 | N-Ph | O | b1 | b1 | H |
| 2-23-769 | N-Ph | O | b2 | b2 | H |
| 2-23-770 | N-Ph | O | b3 | b3 | H |
| 2-23-771 | N-Ph | O | H | H | a1 |
| 2-23-772 | N-Ph | O | t-bu | t-bu | a1 |
| 2-23-773 | N-Ph | O | methyl | methyl | a1 |
| 2-23-774 | N-Ph | O | b1 | b1 | a1 |
| 2-23-775 | N-Ph | O | b2 | b2 | a1 |
| 2-23-776 | N-Ph | O | b3 | b3 | a1 |
| 2-23-777 | N-Ph | O | H | H | a2 |
| 2-23-778 | N-Ph | O | t-bu | t-bu | a2 |
| 2-23-779 | N-Ph | O | methyl | methyl | a2 |
| 2-23-780 | N-Ph | O | b1 | b1 | a2 |
| 2-23-781 | N-Ph | O | b2 | b2 | a2 |
| 2-23-782 | N-Ph | O | b3 | b3 | a2 |
| 2-23-783 | N-Ph | O | H | H | a3 |
| 2-23-784 | N-Ph | O | t-bu | t-bu | a3 |
| 2-23-785 | N-Ph | O | methyl | methyl | a3 |
| 2-23-786 | N-Ph | O | b1 | b1 | a3 |
| 2-23-787 | N-Ph | O | b2 | b2 | a3 |
| 2-23-788 | N-Ph | O | b3 | b3 | a3 |
| 2-23-789 | N-Ph | O | H | H | a4 |
| 2-23-790 | N-Ph | O | t-bu | t-bu | a4 |
| 2-23-791 | N-Ph | O | methyl | methyl | a4 |
| 2-23-792 | N-Ph | O | b1 | b1 | a4 |
| 2-23-793 | N-Ph | O | b2 | b2 | a4 |
| 2-23-794 | N-Ph | O | b3 | b3 | a4 |
| 2-23-795 | N-Ph | O | H | H | a5 |
| 2-23-796 | N-Ph | O | t-bu | t-bu | a5 |
| 2-23-797 | N-Ph | O | methyl | methyl | a5 |
| 2-23-798 | N-Ph | O | b1 | b1 | a5 |
| 2-23-799 | N-Ph | O | b2 | b2 | a5 |
| 2-23-800 | N-Ph | O | b3 | b3 | a5 |
| 2-23-801 | N-Ph | O | H | H | a6 |
| 2-23-802 | N-Ph | O | t-bu | t-bu | a6 |
| 2-23-803 | N-Ph | O | methyl | methyl | a6 |
| 2-23-804 | N-Ph | O | b1 | b1 | a6 |
| 2-23-805 | N-Ph | O | b2 | b2 | a6 |
| 2-23-806 | N-Ph | O | b3 | b3 | a6 |
| 2-23-807 | N-Ph | O | H | H | b1 |
| 2-23-808 | N-Ph | O | t-bu | t-bu | b1 |
| 2-23-809 | N-Ph | O | methyl | methyl | b1 |
| 2-23-810 | N-Ph | O | b1 | b1 | b1 |
| 2-23-811 | N-Ph | O | b2 | b2 | b1 |
| 2-23-812 | N-Ph | O | b3 | b3 | b1 |
| 2-23-813 | N-Ph | O | H | H | b2 |
| 2-23-814 | N-Ph | O | t-bu | t-bu | b2 |
| 2-23-815 | N-Ph | O | methyl | methyl | b2 |
| 2-23-816 | N-Ph | O | b1 | b1 | b2 |
| 2-23-817 | N-Ph | O | b2 | b2 | b2 |
| 2-23-818 | N-Ph | O | b3 | b3 | b2 |
| 2-23-819 | N-Ph | O | H | H | b3 |
| 2-23-820 | N-Ph | O | t-bu | t-bu | b3 |
| 2-23-821 | N-Ph | O | methyl | methyl | b3 |
| 2-23-822 | N-Ph | O | b1 | b1 | b3 |
| 2-23-823 | N-Ph | O | b2 | b2 | b3 |
| 2-23-824 | N-Ph | O | b3 | b3 | b3 |
| 2-23-825 | N-Ph | O | H | H | b4 |
| 2-23-826 | N-Ph | O | t-bu | t-bu | b4 |
| 2-23-827 | N-Ph | O | methyl | methyl | b4 |
| 2-23-828 | N-Ph | O | b1 | b1 | b4 |
| 2-23-829 | N-Ph | O | b2 | b2 | b4 |
| 2-23-830 | N-Ph | O | b3 | b3 | b4 |
| 2-23-831 | N-Ph | O | H | H | b5 |
| 2-23-832 | N-Ph | O | t-bu | t-bu | b5 |
| 2-23-833 | N-Ph | O | methyl | methyl | b5 |
| 2-23-834 | N-Ph | O | b1 | b1 | b5 |
| 2-23-835 | N-Ph | O | b2 | b2 | b5 |
| 2-23-836 | N-Ph | O | b3 | b3 | b5 |
| 2-23-837 | N-Ph | O | H | H | b6 |
| 2-23-838 | N-Ph | O | t-bu | t-bu | b6 |
| 2-23-839 | N-Ph | O | methyl | methyl | b6 |
| 2-23-840 | N-Ph | O | b1 | b1 | b6 |
| 2-23-841 | N-Ph | O | b2 | b2 | b6 |
| 2-23-842 | N-Ph | O | b3 | b3 | b6 |
| 2-23-843 | N-Ph | O | H | H | b7 |
| 2-23-844 | N-Ph | O | t-bu | t-bu | b7 |
| 2-23-845 | N-Ph | O | methyl | methyl | b7 |
| 2-23-846 | N-Ph | O | b1 | b1 | b7 |
| 2-23-847 | N-Ph | O | b2 | b2 | b7 |
| 2-23-848 | N-Ph | O | b3 | b3 | b7 |
| 2-23-849 | N-Ph | O | H | H | b8 |
| 2-23-850 | N-Ph | O | t-bu | t-bu | b8 |
| 2-23-851 | N-Ph | O | methyl | methyl | b8 |
| 2-23-852 | N-Ph | O | b1 | b1 | b8 |
| 2-23-853 | N-Ph | O | b2 | b2 | b8 |
| 2-23-854 | N-Ph | O | b3 | b3 | b8 |
| 2-23-855 | N-Ph | O | H | H | b9 |
| 2-23-856 | N-Ph | O | t-bu | t-bu | b9 |
| 2-23-857 | N-Ph | O | methyl | methyl | b9 |
| 2-23-858 | N-Ph | O | b1 | b1 | b9 |
| 2-23-859 | N-Ph | O | b2 | b2 | b9 |
| 2-23-860 | N-Ph | O | b3 | b3 | b9 |
| 2-23-861 | N-Ph | O | H | H | b10 |
| 2-23-862 | N-Ph | O | t-bu | t-bu | b10 |
| 2-23-863 | N-Ph | O | methyl | methyl | b10 |
| 2-23-864 | N-Ph | O | b1 | b1 | b10 |
| 2-23-865 | N-Ph | O | b2 | b2 | b10 |
| 2-23-866 | N-Ph | O | b3 | b3 | b10 |
| 2-23-867 | N-Ph | O | H | H | H |
| 2-23-868 | N-Ph | O | t-bu | t-bu | H |
| 2-23-869 | N-Ph | O | methyl | methyl | H |
| 2-23-870 | N-Ph | O | b1 | b1 | H |
| 2-23-871 | N-Ph | O | b2 | b2 | H |
| 2-23-872 | N-Ph | O | b3 | b3 | H |
| 2-23-873 | N-Ph | O | H | H | H |
| 2-23-874 | N-Ph | O | t-bu | t-bu | H |
| 2-23-875 | N-Ph | O | methyl | methyl | H |
| 2-23-876 | N-Ph | O | b1 | b1 | H |
| 2-23-877 | N-Ph | O | b2 | b2 | H |
| 2-23-878 | N-Ph | O | b3 | b3 | H |
| 2-23-879 | N-Ph | O | H | H | H |
| 2-23-880 | N-Ph | O | t-bu | t-bu | H |
| 2-23-881 | N-Ph | O | methyl | methyl | H |
| 2-23-882 | N-Ph | O | b1 | b1 | H |
| 2-23-883 | N-Ph | O | b2 | b2 | H |
| 2-23-884 | N-Ph | O | b3 | b3 | H |
| 2-23-885 | N-Ph | O | H | H | H |
| 2-23-886 | N-Ph | O | t-bu | t-bu | H |
| 2-23-887 | N-Ph | O | methyl | methyl | H |
| 2-23-888 | N-Ph | O | b1 | b1 | H |
| 2-23-889 | N-Ph | O | b2 | b2 | H |
| 2-23-890 | N-Ph | O | b3 | b3 | H |
| 2-23-891 | N-Ph | O | H | H | H |
| 2-23-892 | N-Ph | O | t-bu | t-bu | H |
| 2-23-893 | N-Ph | O | methyl | methyl | H |
| 2-23-894 | N-Ph | O | b1 | b1 | H |
| 2-23-895 | N-Ph | O | b2 | b2 | H |
| 2-23-896 | N-Ph | O | b3 | b3 | H |
| 2-23-897 | N-Ph | O | H | H | H |
| 2-23-898 | N-Ph | O | t-bu | t-bu | H |
| 2-23-899 | N-Ph | O | methyl | methyl | H |
| 2-23-900 | N-Ph | O | b1 | b1 | H |
| 2-23-901 | N-Ph | O | b2 | b2 | H |
| 2-23-902 | N-Ph | O | b3 | b3 | H |
| 2-23-903 | N-Ph | O | H | H | H |
| 2-23-904 | N-Ph | O | t-bu | t-bu | H |
| 2-23-905 | N-Ph | O | methyl | methyl | H |
| 2-23-906 | N-Ph | O | b1 | b1 | H |
| 2-23-907 | N-Ph | O | b2 | b2 | H |
| 2-23-908 | N-Ph | O | b3 | b3 | H |
| 2-23-909 | N-Ph | O | H | H | H |
| 2-23-910 | N-Ph | O | t-bu | t-bu | H |
| 2-23-911 | N-Ph | O | methyl | methyl | H |
| 2-23-912 | N-Ph | O | b1 | b1 | H |
| 2-23-913 | N-Ph | O | b2 | b2 | H |
| 2-23-914 | N-Ph | O | b3 | b3 | H |
| 2-23-915 | N-Ph | O | H | H | H |
| 2-23-916 | N-Ph | O | t-bu | t-bu | H |
| 2-23-917 | N-Ph | O | methyl | methyl | H |
| 2-23-918 | N-Ph | O | b1 | b1 | H |
| 2-23-919 | N-Ph | O | b2 | b2 | H |
| 2-23-920 | N-Ph | O | b3 | b3 | H |
| 2-23-921 | N-Ph | O | H | H | H |
| 2-23-922 | N-Ph | O | t-bu | t-bu | H |
| 2-23-923 | N-Ph | O | methyl | methyl | H |
| 2-23-924 | N-Ph | O | b1 | b1 | H |
| 2-23-925 | N-Ph | O | b2 | b2 | H |
| 2-23-926 | N-Ph | O | b3 | b3 | H |
| 2-23-927 | N-Ph | O | H | H | H |
| 2-23-928 | N-Ph | O | t-bu | t-bu | H |
| 2-23-929 | N-Ph | O | methyl | methyl | H |
| 2-23-930 | N-Ph | O | b1 | b1 | H |
| 2-23-931 | N-Ph | O | b2 | b2 | H |
| 2-23-932 | N-Ph | O | b3 | b3 | H |
| 2-23-933 | N-Ph | O | H | H | H |
| 2-23-934 | N-Ph | O | t-bu | t-bu | H |
| 2-23-935 | N-Ph | O | methyl | methyl | H |
| 2-23-936 | N-Ph | O | b1 | b1 | H |
| 2-23-937 | N-Ph | O | b2 | b2 | H |
| 2-23-938 | N-Ph | O | b3 | b3 | H |
| 2-23-939 | N-Ph | O | H | H | H |
| 2-23-940 | N-Ph | O | t-bu | t-bu | H |
| 2-23-941 | N-Ph | O | methyl | methyl | H |
| 2-23-942 | N-Ph | O | b1 | b1 | H |
| 2-23-943 | N-Ph | O | b2 | b2 | H |
| 2-23-944 | N-Ph | O | b3 | b3 | H |
| 2-23-945 | N-Ph | O | H | H | H |
| 2-23-946 | N-Ph | O | t-bu | t-bu | H |
| 2-23-947 | N-Ph | O | methyl | methyl | H |
| 2-23-948 | N-Ph | O | b1 | b1 | H |
| 2-23-949 | N-Ph | O | b2 | b2 | H |
| 2-23-950 | N-Ph | O | b3 | b3 | H |
| 2-23-951 | N-Ph | O | H | H | H |
| 2-23-952 | N-Ph | O | t-bu | t-bu | H |
| 2-23-953 | N-Ph | O | methyl | methyl | H |
| 2-23-954 | N-Ph | O | b1 | b1 | H |
| 2-23-955 | N-Ph | O | b2 | b2 | H |
| 2-23-956 | N-Ph | O | b3 | b3 | H |
| 2-23-957 | N-Ph | O | H | H | H |
| 2-23-958 | N-Ph | O | t-bu | t-bu | H |
| 2-23-959 | N-Ph | O | methyl | methyl | H |
| 2-23-960 | N-Ph | O | b1 | b1 | H |
| 2-23-961 | N-Ph | O | b2 | b2 | H |
| 2-23-962 | N-Ph | O | b3 | b3 | H |
| 2-23-963 | N-Ph | O | H | H | a1 |
| 2-23-964 | N-Ph | O | t-bu | t-bu | a1 |
| 2-23-965 | N-Ph | O | methyl | methyl | a1 |
| 2-23-966 | N-Ph | O | b1 | b1 | a1 |
| 2-23-967 | N-Ph | O | b2 | b2 | a1 |
| 2-23-968 | N-Ph | O | b3 | b3 | a1 |
| 2-23-969 | N-Ph | O | H | H | a2 |
| 2-23-970 | N-Ph | O | t-bu | t-bu | a2 |
| 2-23-971 | N-Ph | O | methyl | methyl | a2 |
| 2-23-972 | N-Ph | O | b1 | b1 | a2 |
| 2-23-973 | N-Ph | O | b2 | b2 | a2 |
| 2-23-974 | N-Ph | O | b3 | b3 | a2 |
| 2-23-975 | N-Ph | O | H | H | a3 |
| 2-23-976 | N-Ph | O | t-bu | t-bu | a3 |
| 2-23-977 | N-Ph | O | methyl | methyl | a3 |
| 2-23-978 | N-Ph | O | b1 | b1 | a3 |
| 2-23-979 | N-Ph | O | b2 | b2 | a3 |
| 2-23-980 | N-Ph | O | b3 | b3 | a3 |
| 2-23-981 | N-Ph | O | H | H | b1 |
| 2-23-982 | N-Ph | O | t-bu | t-bu | b1 |
| 2-23-983 | N-Ph | O | methyl | methyl | b1 |
| 2-23-984 | N-Ph | O | b1 | b1 | b1 |
| 2-23-985 | N-Ph | O | b2 | b2 | b1 |
| 2-23-986 | N-Ph | O | b3 | b3 | b1 |
| 2-23-987 | N-Ph | O | H | H | b2 |
| 2-23-988 | N-Ph | O | t-bu | t-bu | b2 |
| 2-23-989 | N-Ph | O | methyl | methyl | b2 |
| 2-23-990 | N-Ph | O | b1 | b1 | b2 |
| 2-23-991 | N-Ph | O | b2 | b2 | b2 |
| 2-23-992 | N-Ph | O | b3 | b3 | b2 |
| 2-23-993 | N-Ph | O | H | H | b3 |
| 2-23-994 | N-Ph | O | t-bu | t-bu | b3 |
| 2-23-995 | N-Ph | O | methyl | methyl | b3 |
| 2-23-996 | N-Ph | O | b1 | b1 | b3 |
| 2-23-997 | N-Ph | O | b2 | b2 | b3 |
| 2-23-998 | N-Ph | O | b3 | b3 | b3 |
| 2-23-999 | N-Ph | O | H | H | b5 |
| 2-23-1000 | N-Ph | O | t-bu | t-bu | b5 |
| 2-23-1001 | N-Ph | O | methyl | methyl | b5 |
| 2-23-1002 | N-Ph | O | b1 | b1 | b5 |
| 2-23-1003 | N-Ph | O | b2 | b2 | b5 |
| 2-23-1004 | N-Ph | O | b3 | b3 | b5 |
| 2-23-1005 | N-Ph | O | H | H | b6 |
| 2-23-1006 | N-Ph | O | t-bu | t-bu | b6 |
| 2-23-1007 | N-Ph | O | methyl | methyl | b6 |
| 2-23-1008 | N-Ph | O | b1 | b1 | b6 |
| 2-23-1009 | N-Ph | O | b2 | b2 | b6 |
| 2-23-1010 | N-Ph | O | b3 | b3 | b6 |
| 2-23-1011 | N-Ph | O | H | H | b7 |
| 2-23-1012 | N-Ph | O | t-bu | t-bu | b7 |
| 2-23-1013 | N-Ph | O | methyl | methyl | b7 |
| 2-23-1014 | N-Ph | O | b1 | b1 | b7 |
| 2-23-1015 | N-Ph | O | b2 | b2 | b7 |
| 2-23-1016 | N-Ph | O | b3 | b3 | b7 |
| 2-23-1017 | N-Ph | S | H | H | H |
| 2-23-1018 | N-Ph | S | t-bu | t-bu | H |
| 2-23-1019 | N-Ph | S | methyl | methyl | H |
| 2-23-1020 | N-Ph | S | t-bu | t-bu | t-bu |
| 2-23-1021 | N-Ph | S | methyl | methyl | methyl |
| 2-23-1022 | N-Ph | S | b1 | b1 | H |
| 2-23-1023 | N-Ph | S | b2 | b2 | H |
| 2-23-1024 | N-Ph | S | b3 | b3 | H |
| 2-23-1025 | N-Ph | S | H | H | a1 |
| 2-23-1026 | N-Ph | S | t-bu | t-bu | a1 |
| 2-23-1027 | N-Ph | S | methyl | methyl | a1 |
| 2-23-1028 | N-Ph | S | b1 | b1 | a1 |
| 2-23-1029 | N-Ph | S | b2 | b2 | a1 |
| 2-23-1030 | N-Ph | S | b3 | b3 | a1 |
| 2-23-1031 | N-Ph | S | H | H | a2 |
| 2-23-1032 | N-Ph | S | t-bu | t-bu | a2 |
| 2-23-1033 | N-Ph | S | methyl | methyl | a2 |
| 2-23-1034 | N-Ph | S | b1 | b1 | a2 |
| 2-23-1035 | N-Ph | S | b2 | b2 | a2 |
| 2-23-1036 | N-Ph | S | b3 | b3 | a2 |
| 2-23-1037 | N-Ph | S | H | H | a3 |
| 2-23-1038 | N-Ph | S | t-bu | t-bu | a3 |
| 2-23-1039 | N-Ph | S | methyl | methyl | a3 |
| 2-23-1040 | N-Ph | S | b1 | b1 | a3 |
| 2-23-1041 | N-Ph | S | b2 | b2 | a3 |
| 2-23-1042 | N-Ph | S | b3 | b3 | a3 |
| 2-23-1043 | N-Ph | S | H | H | a4 |
| 2-23-1044 | N-Ph | S | t-bu | t-bu | a4 |
| 2-23-1045 | N-Ph | S | methyl | methyl | a4 |
| 2-23-1046 | N-Ph | S | b1 | b1 | a4 |
| 2-23-1047 | N-Ph | S | b2 | b2 | a4 |
| 2-23-1048 | N-Ph | S | b3 | b3 | a4 |
| 2-23-1049 | N-Ph | S | H | H | a5 |
| 2-23-1050 | N-Ph | S | t-bu | t-bu | a5 |
| 2-23-1051 | N-Ph | S | methyl | methyl | a5 |
| 2-23-1052 | N-Ph | S | b1 | b1 | a5 |
| 2-23-1053 | N-Ph | S | b2 | b2 | a5 |
| 2-23-1054 | N-Ph | S | b3 | b3 | a5 |
| 2-23-1055 | N-Ph | S | H | H | a6 |
| 2-23-1056 | N-Ph | S | t-bu | t-bu | a6 |
| 2-23-1057 | N-Ph | S | methyl | methyl | a6 |
| 2-23-1058 | N-Ph | S | b1 | b1 | a6 |
| 2-23-1059 | N-Ph | S | b2 | b2 | a6 |
| 2-23-1060 | N-Ph | S | b3 | b3 | a6 |
| 2-23-1061 | N-Ph | S | H | H | b1 |
| 2-23-1062 | N-Ph | S | t-bu | t-bu | b1 |
| 2-23-1063 | N-Ph | S | methyl | methyl | b1 |
| 2-23-1064 | N-Ph | S | b1 | b1 | b1 |
| 2-23-1065 | N-Ph | S | b2 | b2 | b1 |
| 2-23-1066 | N-Ph | S | b3 | b3 | b1 |
| 2-23-1067 | N-Ph | S | H | H | b2 |
| 2-23-1068 | N-Ph | S | t-bu | t-bu | b2 |
| 2-23-1069 | N-Ph | S | methyl | methyl | b2 |
| 2-23-1070 | N-Ph | S | b1 | b1 | b2 |
| 2-23-1071 | N-Ph | S | b2 | b2 | b2 |
| 2-23-1072 | N-Ph | S | b3 | b3 | b2 |
| 2-23-1073 | N-Ph | S | H | H | b3 |
| 2-23-1074 | N-Ph | S | t-bu | t-bu | b3 |
| 2-23-1075 | N-Ph | S | methyl | methyl | b3 |
| 2-23-1076 | N-Ph | S | b1 | b1 | b3 |
| 2-23-1077 | N-Ph | S | b2 | b2 | b3 |
| 2-23-1078 | N-Ph | S | b3 | b3 | b3 |
| 2-23-1079 | N-Ph | S | H | H | b4 |
| 2-23-1080 | N-Ph | S | t-bu | t-bu | b4 |
| 2-23-1081 | N-Ph | S | methyl | methyl | b4 |
| 2-23-1082 | N-Ph | S | b1 | b1 | b4 |
| 2-23-1083 | N-Ph | S | b2 | b2 | b4 |
| 2-23-1084 | N-Ph | S | b3 | b3 | b4 |
| 2-23-1085 | N-Ph | S | H | H | b5 |
| 2-23-1086 | N-Ph | S | t-bu | t-bu | b5 |
| 2-23-1087 | N-Ph | S | methyl | methyl | b5 |
| 2-23-1088 | N-Ph | S | b1 | b1 | b5 |
| 2-23-1089 | N-Ph | S | b2 | b2 | b5 |
| 2-23-1090 | N-Ph | S | b3 | b3 | b5 |
| 2-23-1091 | N-Ph | S | H | H | b6 |
| 2-23-1092 | N-Ph | S | t-bu | t-bu | b6 |
| 2-23-1093 | N-Ph | S | methyl | methyl | b6 |
| 2-23-1094 | N-Ph | S | b1 | b1 | b6 |
| 2-23-1095 | N-Ph | S | b2 | b2 | b6 |
| 2-23-1096 | N-Ph | S | b3 | b3 | b6 |
| 2-23-1097 | N-Ph | S | H | H | b7 |
| 2-23-1098 | N-Ph | S | t-bu | t-bu | b7 |
| 2-23-1099 | N-Ph | S | methyl | methyl | b7 |
| 2-23-1100 | N-Ph | S | b1 | b1 | b7 |
| 2-23-1101 | N-Ph | S | b2 | b2 | b7 |
| 2-23-1102 | N-Ph | S | b3 | b3 | b7 |
| 2-23-1103 | N-Ph | S | H | H | b8 |
| 2-23-1104 | N-Ph | S | t-bu | t-bu | b8 |
| 2-23-1105 | N-Ph | S | methyl | methyl | b8 |
| 2-23-1106 | N-Ph | S | b1 | b1 | b8 |
| 2-23-1107 | N-Ph | S | b2 | b2 | b8 |
| 2-23-1108 | N-Ph | S | b3 | b3 | b8 |
| 2-23-1109 | N-Ph | S | H | H | b9 |
| 2-23-1110 | N-Ph | S | t-bu | t-bu | b9 |
| 2-23-1111 | N-Ph | S | methyl | methyl | b9 |
| 2-23-1112 | N-Ph | S | b1 | b1 | b9 |
| 2-23-1113 | N-Ph | S | b2 | b2 | b9 |
| 2-23-1114 | N-Ph | S | b3 | b3 | b9 |
| 2-23-1115 | N-Ph | S | H | H | b10 |
| 2-23-1116 | N-Ph | S | t-bu | t-bu | b10 |
| 2-23-1117 | N-Ph | S | methyl | methyl | b10 |
| 2-23-1118 | N-Ph | S | b1 | b1 | b10 |
| 2-23-1119 | N-Ph | S | b2 | b2 | b10 |
| 2-23-1120 | N-Ph | S | b3 | b3 | b10 |
| 2-23-1121 | N-Ph | S | H | H | H |
| 2-23-1122 | N-Ph | S | t-bu | t-bu | H |
| 2-23-1123 | N-Ph | S | methyl | methyl | H |
| 2-23-1124 | N-Ph | S | b1 | b1 | H |
| 2-23-1125 | N-Ph | S | b2 | b2 | H |
| 2-23-1126 | N-Ph | S | b3 | b3 | H |
| 2-23-1127 | N-Ph | S | H | H | H |
| 2-23-1128 | N-Ph | S | t-bu | t-bu | H |
| 2-23-1129 | N-Ph | S | methyl | methyl | H |
| 2-23-1130 | N-Ph | S | b1 | b1 | H |
| 2-23-1131 | N-Ph | S | b2 | b2 | H |
| 2-23-1132 | N-Ph | 5 | b3 | b3 | H |
| 2-23-1133 | N-Ph | S | H | H | H |
| 2-23-1134 | N-Ph | S | t-bu | t-bu | H |
| 2-23-1135 | N-Ph | S | methyl | methyl | H |
| 2-23-1136 | N-Ph | S | b1 | b1 | H |
| 2-23-1137 | N-Ph | S | b2 | b2 | H |
| 2-23-1138 | N-Ph | S | b3 | b3 | H |
| 2-23-1139 | N-Ph | S | H | H | H |
| 2-23-1140 | N-Ph | S | t-bu | t-bu | H |
| 2-23-1141 | N-Ph | S | methyl | methyl | H |
| 2-23-1142 | N-Ph | S | b1 | b1 | H |
| 2-23-1143 | N-Ph | S | b2 | b2 | H |
| 2-23-1144 | N-Ph | S | b3 | b3 | H |
| 2-23-1145 | N-Ph | S | H | H | H |
| 2-23-1146 | N-Ph | S | t-bu | t-bu | H |
| 2-23-1147 | N-Ph | S | methyl | methyl | H |
| 2-23-1148 | N-Ph | S | b1 | b1 | H |
| 2-23-1149 | N-Ph | S | b2 | b2 | H |
| 2-23-1150 | N-Ph | S | b3 | b3 | H |
| 2-23-1151 | N-Ph | S | H | H | H |
| 2-23-1152 | N-Ph | S | t-bu | t-bu | H |
| 2-23-1153 | N-Ph | S | methyl | methyl | H |
| 2-23-1154 | N-Ph | S | b1 | b1 | H |
| 2-23-1155 | N-Ph | S | b2 | b2 | H |
| 2-23-1156 | N-Ph | S | b3 | b3 | H |
| 2-23-1157 | N-Ph | S | H | H | H |
| 2-23-1158 | N-Ph | S | t-bu | t-bu | H |
| 2-23-1159 | N-Ph | S | methyl | methyl | H |
| 2-23-1160 | N-Ph | S | b1 | b1 | H |
| 2-23-1161 | N-Ph | S | b2 | b2 | H |
| 2-23-1162 | N-Ph | S | b3 | b3 | H |
| 2-23-1163 | N-Ph | S | H | H | H |
| 2-23-1164 | N-Ph | S | t-bu | t-bu | H |
| 2-23-1165 | N-Ph | S | methyl | methyl | H |
| 2-23-1166 | N-Ph | S | b1 | b1 | H |
| 2-23-1167 | N-Ph | S | b2 | b2 | H |
| 2-23-1168 | N-Ph | S | b3 | b3 | H |
| 2-23-1169 | N-Ph | S | H | H | H |
| 2-23-1170 | N-Ph | S | t-bu | t-bu | H |
| 2-23-1171 | N-Ph | S | methyl | methyl | H |
| 2-23-1172 | N-Ph | S | b1 | b1 | H |
| 2-23-1173 | N-Ph | S | b2 | b2 | H |
| 2-23-1174 | N-Ph | S | b3 | b3 | H |
| 2-23-1175 | N-Ph | S | H | H | H |
| 2-23-1176 | N-Ph | S | t-bu | t-bu | H |
| 2-23-1177 | N-Ph | S | methyl | methyl | H |
| 2-23-1178 | N-Ph | S | b1 | b1 | H |
| 2-23-1179 | N-Ph | S | b2 | b2 | H |
| 2-23-1180 | N-Ph | S | b3 | b3 | H |
| 2-23-1181 | N-Ph | S | H | H | H |
| 2-23-1182 | N-Ph | S | t-bu | t-bu | H |
| 2-23-1183 | N-Ph | S | methyl | methyl | H |
| 2-23-1184 | N-Ph | S | b1 | b1 | H |
| 2-23-1185 | N-Ph | S | b2 | b2 | H |
| 2-23-1186 | N-Ph | S | b3 | b3 | H |
| 2-23-1187 | N-Ph | S | H | H | H |
| 2-23-1188 | N-Ph | S | t-bu | t-bu | H |
| 2-23-1189 | N-Ph | S | methyl | methyl | H |
| 2-23-1190 | N-Ph | S | b1 | b1 | H |
| 2-23-1191 | N-Ph | S | b2 | b2 | H |
| 2-23-1192 | N-Ph | S | b3 | b3 | H |
| 2-23-1193 | N-Ph | S | H | H | H |
| 2-23-1194 | N-Ph | S | t-bu | t-bu | H |
| 2-23-1195 | N-Ph | S | methyl | methyl | H |
| 2-23-1196 | N-Ph | S | b1 | b1 | H |
| 2-23-1197 | N-Ph | S | b2 | b2 | H |
| 2-23-1198 | N-Ph | S | b3 | b3 | H |
| 2-23-1199 | N-Ph | S | H | H | H |
| 2-23-1200 | N-Ph | S | t-bu | t-bu | H |
| 2-23-1201 | N-Ph | S | methyl | methyl | H |
| 2-23-1202 | N-Ph | S | b1 | b1 | H |
| 2-23-1203 | N-Ph | S | b2 | b2 | H |
| 2-23-1204 | N-Ph | S | b3 | b3 | H |
| 2-23-1205 | N-Ph | S | H | H | H |
| 2-23-1206 | N-Ph | S | t-bu | t-bu | H |
| 2-23-1207 | N-Ph | S | methyl | methyl | H |
| 2-23-1208 | N-Ph | S | b1 | b1 | H |
| 2-23-1209 | N-Ph | S | b2 | b2 | H |
| 2-23-1210 | N-Ph | S | b3 | b3 | H |
| 2-23-1211 | N-Ph | S | H | H | H |
| 2-23-1212 | N-Ph | S | t-bu | t-bu | H |
| 2-23-1213 | N-Ph | S | methyl | methyl | H |
| 2-23-1214 | N-Ph | S | b1 | b1 | H |
| 2-23-1215 | N-Ph | S | b2 | b2 | H |
| 2-23-1216 | N-Ph | S | b3 | b3 | H |
| 2-23-1217 | N-Ph | S | H | H | a1 |
| 2-23-1218 | N-Ph | S | t-bu | t-bu | a1 |
| 2-23-1219 | N-Ph | S | methyl | methyl | a1 |
| 2-23-1220 | N-Ph | S | b1 | b1 | a1 |
| 2-23-1221 | N-Ph | S | b2 | b2 | a1 |
| 2-23-1222 | N-Ph | S | b3 | b3 | a1 |
| 2-23-1223 | N-Ph | S | H | H | a2 |
| 2-23-1224 | N-Ph | S | t-bu | t-bu | a2 |
| 2-23-1225 | N-Ph | S | methyl | methyl | a2 |
| 2-23-1226 | N-Ph | S | b1 | b1 | a2 |
| 2-23-1227 | N-Ph | S | b2 | b2 | a2 |
| 2-23-1228 | N-Ph | S | b3 | b3 | a2 |
| 2-23-1229 | N-Ph | S | H | H | a3 |
| 2-23-1230 | N-Ph | S | t-bu | t-bu | a3 |
| 2-23-1231 | N-Ph | S | methyl | methyl | a3 |
| 2-23-1232 | N-Ph | S | b1 | b1 | a3 |
| 2-23-1233 | N-Ph | S | b2 | b2 | a3 |
| 2-23-1234 | N-Ph | S | b3 | b3 | a3 |
| 2-23-1235 | N-Ph | S | H | H | b1 |
| 2-23-1236 | N-Ph | S | t-bu | t-bu | b1 |
| 2-23-1237 | N-Ph | S | methyl | methyl | b1 |
| 2-23-1238 | N-Ph | S | b1 | b1 | b1 |
| 2-23-1239 | N-Ph | S | b2 | b2 | b1 |
| 2-23-1240 | N-Ph | S | b3 | b3 | b1 |
| 2-23-1241 | N-Ph | S | H | H | b2 |
| 2-23-1242 | N-Ph | S | t-bu | t-bu | b2 |
| 2-23-1243 | N-Ph | S | methyl | methyl | b2 |
| 2-23-1244 | N-Ph | S | b1 | b1 | b2 |
| 2-23-1245 | N-Ph | S | b2 | b2 | b2 |
| 2-23-1246 | N-Ph | S | b3 | b3 | b2 |
| 2-23-1247 | N-Ph | S | H | H | b3 |
| 2-23-1248 | N-Ph | S | t-bu | t-bu | b3 |
| 2-23-1249 | N-Ph | S | methyl | methyl | b3 |
| 2-23-1250 | N-Ph | S | b1 | b1 | b3 |
| 2-23-1251 | N-Ph | S | b2 | b2 | b3 |
| 2-23-1252 | N-Ph | S | b3 | b3 | b3 |
| 2-23-1253 | N-Ph | S | H | H | b5 |
| 2-23-1254 | N-Ph | S | t-bu | t-bu | b5 |
| 2-23-1255 | N-Ph | S | methyl | methyl | b5 |
| 2-23-1256 | N-Ph | S | b1 | b1 | b5 |
| 2-23-1257 | N-Ph | S | b2 | b2 | b5 |
| 2-23-1258 | N-Ph | S | b3 | b3 | b5 |
| 2-23-1259 | N-Ph | S | H | H | b6 |
| 2-23-1260 | N-Ph | S | t-bu | t-bu | b6 |
| 2-23-1261 | N-Ph | S | methyl | methyl | b6 |
| 2-23-1262 | N-Ph | S | b1 | b1 | b6 |
| 2-23-1263 | N-Ph | S | b2 | b2 | b6 |
| 2-23-1264 | N-Ph | S | b3 | b3 | b6 |
| 2-23-1265 | N-Ph | S | H | H | b7 |
| 2-23-1266 | N-Ph | S | t-bu | t-bu | b7 |
| 2-23-1267 | N-Ph | S | methyl | methyl | b7 |
| 2-23-1268 | N-Ph | S | b1 | b1 | b7 |
| 2-23-1269 | N-Ph | S | b2 | b2 | b7 |
| 2-23-1270 | N-Ph | S | b3 | b3 | b7 |

### Group 2-24

The compounds are represented by the formula 2-24 below, wherein X₁ is B, and Y1, Y2, Y3, R1b, R2b, R3a are each shown in the table 24 below,

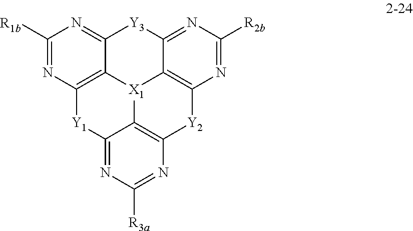

TABLE 24

| Compound | | | | |
|---|---|---|---|---|
| No. | Y1/Y2/Y3 | R1b | R2b | R3a |
| 2-24-001 | N-Ph | H | H | H |
| 2-24-002 | N-Ph | t-bu | t-bu | t-bu |
| 2-24-003 | N-Ph | methyl | methyl | methyl |
| 2-24-004 | N-Ph | b1 | b1 | b1 |
| 2-24-005 | N-Ph | b2 | b2 | b2 |
| 2-24-006 | N-Ph | b3 | b3 | b3 |
| 2-24-007 | N-Ph | b4 | b4 | b4 |
| 2-24-008 | N-Ph | b5 | b5 | b5 |
| 2-24-009 | N-Ph | b6 | b6 | b6 |
| 2-24-010 | N-Ph | b7 | b7 | b7 |
| 2-24-011 | N-Ph | b8 | b8 | b8 |
| 2-24-012 | N-Ph | b9 | b9 | b9 |
| 2-24-013 | N-Ph | b10 | b10 | b10 |
| 2-24-014 | N-Ph | a1 | a1 | a1 |
| 2-24-015 | N-Ph | a2 | a2 | a2 |
| 2-24-016 | N-Ph | a3 | a3 | a3 |
| 2-24-017 | N-Ph | a4 | a4 | a4 |
| 2-24-018 | N-Ph | a5 | a5 | a5 |
| 2-24-019 | N-Ph | a6 | a6 | a6 |
| 2-24-020 | N-tolyl | H | H | H |
| 2-24-021 | N-tolyl | t-bu | t-bu | t-bu |
| 2-24-022 | N-tolyl | methyl | methyl | methyl |
| 2-24-023 | N-tolyl | b1 | b1 | b1 |
| 2-24-024 | N-tolyl | b2 | b2 | b2 |
| 2-24-025 | N-tolyl | b3 | b3 | b3 |
| 2-24-026 | N-tolyl | b4 | b4 | b4 |
| 2-24-027 | N-tolyl | b5 | b5 | b5 |
| 2-24-028 | N-tolyl | b6 | b6 | b6 |
| 2-24-029 | N-tolyl | b7 | b7 | b7 |
| 2-24-030 | N-tolyl | b8 | b8 | b8 |
| 2-24-031 | N-tolyl | b9 | b9 | b9 |
| 2-24-032 | N-tolyl | b10 | b10 | b10 |
| 2-24-033 | N-tolyl | a1 | a1 | a1 |
| 2-24-034 | N-tolyl | a2 | a2 | a2 |
| 2-24-035 | N-tolyl | a3 | a3 | a3 |
| 2-24-036 | N-tolyl | a4 | a4 | a4 |
| 2-24-037 | N-tolyl | a5 | a5 | a5 |
| 2-24-038 | N-tolyl | a6 | a6 | a6 |
| 2-24-039 | N-Biph | H | H | H |
| 2-24-040 | N-Biph | t-bu | t-bu | t-bu |
| 2-24-041 | N-Biph | methyl | methyl | methyl |
| 2-24-042 | N-Biph | b1 | b1 | b1 |
| 2-24-043 | N-Biph | b2 | b2 | b2 |
| 2-24-044 | N-Biph | b3 | b3 | b3 |
| 2-24-045 | N-Biph | b4 | b4 | b4 |
| 2-24-046 | N-Biph | b5 | b5 | b5 |
| 2-24-047 | N-Biph | b6 | b6 | b6 |
| 2-24-048 | N-Biph | b7 | b7 | b7 |
| 2-24-049 | N-Biph | b8 | b8 | b8 |
| 2-24-050 | N-Biph | b9 | b9 | b9 |
| 2-24-051 | N-Biph | b10 | b10 | b10 |
| 2-24-052 | N-Biph | a1 | a1 | a1 |
| 2-24-053 | N-Biph | a2 | a2 | a2 |
| 2-24-054 | N-Biph | a3 | a3 | a3 |
| 2-24-055 | N-Biph | a4 | a4 | a4 |
| 2-24-056 | N-Biph | a5 | a5 | a5 |
| 2-24-057 | N-Biph | a6 | a6 | a6 |
| 2-24-058 | Se | H | H | H |
| 2-24-059 | Se | t-bu | t-bu | t-bu |
| 2-24-060 | Se | methyl | methyl | methyl |
| 2-24-061 | Se | b1 | b1 | b1 |
| 2-24-062 | Se | b2 | b2 | b2 |
| 2-24-063 | Se | b3 | b3 | b3 |
| 2-24-064 | Se | b4 | b4 | b4 |
| 2-24-065 | Se | b5 | b5 | b5 |
| 2-24-066 | Se | b6 | b6 | b6 |
| 2-24-067 | Se | b7 | b7 | b7 |
| 2-24-068 | Se | b8 | b8 | b8 |
| 2-24-069 | Se | b9 | b9 | b9 |
| 2-24-070 | Se | b10 | b10 | b10 |
| 2-24-071 | Se | a1 | a1 | a1 |
| 2-24-072 | Se | a2 | a2 | a2 |
| 2-24-073 | Se | a3 | a3 | a3 |
| 2-24-074 | Se | a4 | a4 | a4 |
| 2-24-075 | Se | a5 | a5 | a5 |
| 2-24-076 | Se | a6 | a6 | a6 |
| 2-24-077 | PO | H | H | H |
| 2-24-078 | PO | t-bu | t-bu | t-bu |
| 2-24-079 | PO | methyl | methyl | methyl |
| 2-24-080 | PO | b1 | b1 | b1 |
| 2-24-081 | PO | b2 | b2 | b2 |
| 2-24-082 | PO | b3 | b3 | b3 |
| 2-24-083 | PO | b4 | b4 | b4 |
| 2-24-084 | PO | b5 | b5 | b5 |
| 2-24-085 | PO | b6 | b6 | b6 |
| 2-24-086 | PO | b7 | b7 | b7 |
| 2-24-087 | PO | b8 | b8 | b8 |
| 2-24-088 | PO | b9 | b9 | b9 |
| 2-24-089 | PO | b10 | b10 | b10 |
| 2-24-090 | PO | a1 | a1 | a1 |
| 2-24-091 | PO | a2 | a2 | a2 |
| 2-24-092 | PO | a3 | a3 | a3 |
| 2-24-093 | PO | a4 | a4 | a4 |
| 2-24-094 | PO | a5 | a5 | a5 |
| 2-24-095 | PO | a6 | a6 | a6 |

### Group 2-25

The compounds are represented by the formula 2-25 below, wherein X₁ is B, and R1 b, R1c, R1d, R2b, R2c, R2d, R3a, R10a, R10b, R10c, R10d, R10e, R10f, R10g, R10h are each shown in the table 25 below,

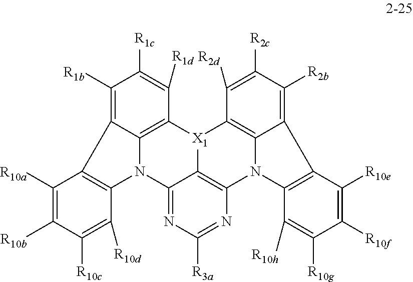

TABLE 25

| Compound | R1c/R1d/ | R2c/R2d/ | | R1b/R2b/R10a/ |
|---|---|---|---|---|
| No. | R10b/R10c | R10f/R10g | R3a | R10d/R10e/R10h |
| 2-25-001 | H | H | H | H |
| 2-25-002 | t-bu | H | H | H |
| 2-25-003 | methyl | H | H | H |
| 2-25-004 | b1 | H | H | H |
| 2-25-005 | b2 | H | H | H |
| 2-25-006 | b3 | H | H | H |
| 2-25-007 | b4 | H | H | H |
| 2-25-008 | b5 | H | H | H |
| 2-25-009 | b6 | H | H | H |
| 2-25-010 | b7 | H | H | H |
| 2-25-011 | b8 | H | H | H |
| 2-25-012 | b9 | H | H | H |
| 2-25-013 | b10 | H | H | H |
| 2-25-014 | a1 | H | H | H |
| 2-25-015 | a2 | H | H | H |
| 2-25-016 | a3 | H | H | H |
| 2-25-017 | a4 | H | H | H |
| 2-25-018 | a5 | H | H | H |
| 2-25-019 | a6 | H | H | H |
| 2-25-020 | H | H | H | H |
| 2-25-021 | H | t-bu | H | H |
| 2-25-022 | H | methyl | H | H |
| 2-25-023 | H | b1 | H | H |
| 2-25-024 | H | b2 | H | H |
| 2-25-025 | H | b3 | H | H |
| 2-25-026 | H | b4 | H | H |
| 2-25-027 | H | b5 | H | H |
| 2-25-028 | H | b6 | H | H |
| 2-25-029 | H | b7 | H | H |
| 2-25-030 | H | b8 | H | H |
| 2-25-031 | H | b9 | H | H |
| 2-25-032 | H | b10 | H | H |
| 2-25-033 | H | a1 | H | H |
| 2-25-034 | H | a2 | H | H |
| 2-25-035 | H | a3 | H | H |
| 2-25-036 | H | a4 | H | H |
| 2-25-037 | H | a5 | H | H |
| 2-25-038 | H | a6 | H | H |
| 2-25-039 | H | H | H | H |
| 2-25-040 | H | H | t-bu | H |
| 2-25-041 | H | H | methyl | H |
| 2-25-042 | H | H | b1 | H |
| 2-25-043 | H | H | b2 | H |
| 2-25-044 | H | H | b3 | H |
| 2-25-045 | H | H | b4 | H |
| 2-25-046 | H | H | b5 | H |
| 2-25-047 | H | H | b6 | H |
| 2-25-048 | H | H | b7 | H |
| 2-25-049 | H | H | b8 | H |
| 2-25-050 | H | H | b9 | H |
| 2-25-051 | H | H | b10 | H |
| 2-25-052 | H | H | a1 | H |
| 2-25-053 | H | H | a2 | H |
| 2-25-054 | H | H | a3 | H |
| 2-25-055 | H | H | a4 | H |
| 2-25-056 | H | H | a5 | H |
| 2-25-057 | H | H | a6 | H |
| 2-25-058 | H | H | t-bu | H |
| 2-25-059 | t-bu | H | methyl | H |
| 2-25-060 | methyl | H | b1 | H |
| 2-25-061 | b1 | H | b2 | H |
| 2-25-062 | b2 | H | b3 | H |
| 2-25-063 | b3 | H | H | H |
| 2-25-064 | b4 | H | t-bu | H |
| 2-25-065 | b5 | H | methyl | H |
| 2-25-066 | b6 | H | b1 | H |
| 2-25-067 | b7 | H | b2 | H |
| 2-25-068 | b8 | H | b3 | H |
| 2-25-069 | b9 | H | H | H |
| 2-25-070 | b10 | H | t-bu | H |
| 2-25-071 | a1 | H | methyl | H |
| 2-25-072 | a2 | H | b1 | H |
| 2-25-073 | a3 | H | b2 | H |
| 2-25-074 | a4 | H | b3 | H |
| 2-25-075 | a5 | H | H | H |
| 2-25-076 | a6 | H | t-bu | H |

wherein in the tables 1 to 25 regarding the groups 2-1 to 2-25 above, “t-bu” indicates “tert-butyl group” and “N-Ph”, “N-tolyl”, “N-Biph”, “a1”, “a2”, “a3”, “a4”, “a5”, “a6”, “b1”, “b2”, “b3”, “b4”, “b5”, “b6”, “b7”, “b8”, “b9”, and “b10” indicates as shown below, and Y indicates Y₁, Y₂ or Y₃:

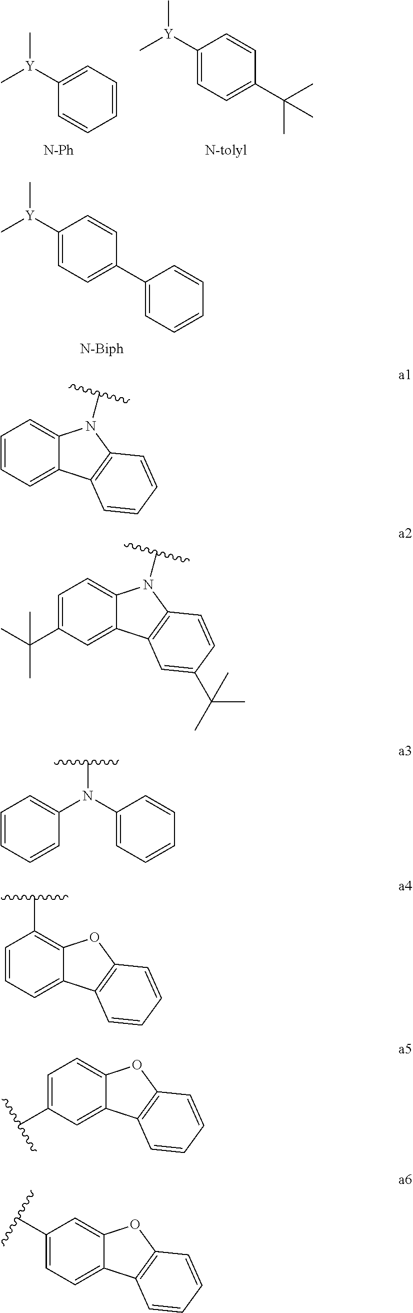

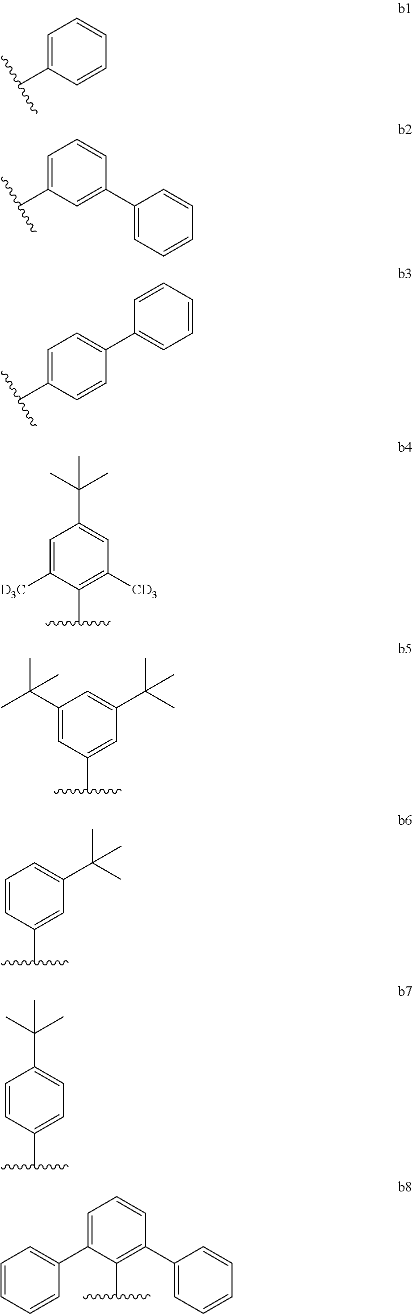

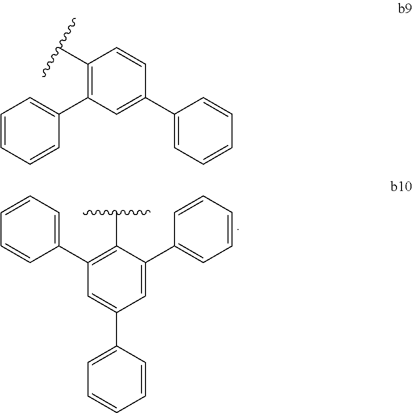

In an embodiment, the heterocyclic compound represented by Formula 1-1 or 1-2, at least one of ring CY₁ to ring CY₃ includes a pyrimidine group. While not wishing to be bound by theory, it is understood that when at least one of ring CY₁ to ring CY₃ includes a pyrimidine group, the multi-resonance effect is improved and the ΔEst value is remarkably reduced, resulting in high efficiency and the color purity effect.

Synthesis method of the heterocyclic compound represented by Formula 1-1 or 1-2 may be recognized by those skilled in the art with reference to the Synthesis Examples provided herein.

In an embodiment, the heterocyclic compound represented by Formula 1-1 or 1-2 is used in an organic layer of an organic light-emitting device, for example, used as a dopant of an emission layer of the organic layer. Another aspect of the present disclosure provides an organic light-emitting device including a first electrode, a second electrode, and an organic layer which is located between the first electrode and the second electrode, includes an emission layer, and at least one of heterocyclic compounds represented by Formula 1-1 or 1-2.

The organic light-emitting device may include an organic layer including the heterocyclic compound represented by Formula 1-1 or 1-2 as described above. In an embodiment, such an organic light-emitting device may have low driving voltage, high or improved efficiency, high or improved power, high or improved quantum efficiency, long or improved lifespan, a low roll-off ratio, and excellent or improved color purity.

The heterocyclic compound represented by Formula 1-1 or 1-2 may be used between a pair of electrodes in an organic light-emitting device. For example, the heterocyclic compound represented by Formula 1-1 or 1-2 may be included in the emission layer. In an embodiment, the heterocyclic compound may act as a dopant, and the emission layer may further include a host (that is, the amount of heterocyclic compound represented by Formula 1-1 or 1-2 may be less than the amount of the host).

The expression “an organic layer includes at least one heterocyclic compound” as used herein may include a case in which “an organic layer includes identical heterocyclic compounds represented by Formula 1-1 or 1-2” and a case in which “an organic layer includes two or more different heterocyclic compounds represented by Formula 1-1 or 1-2”.

For example, the organic layer may include, as the heterocyclic compound, only Compound 1. In an embodiment, Compound 1 may be included in the emission layer of the organic light-emitting device. In an embodiment, the organic layer may include, as the heterocyclic compound, Compound 1 and Compound 2. In an embodiment, Compound 1 and Compound 2 may exist in an identical layer (for example, Compound 1 and Compound 2 all may exist in an emission layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

In an embodiment, in the organic light-emitting device, the first electrode is an anode, and the second electrode is a cathode, and the organic layer further includes a hole transport region between the first electrode and the emission layer and an electron transport region between the emission layer and the second electrode, and the hole transport region includes a hole injection layer, a hole transport layer, an electron blocking layer, or any combination thereof, and the electron transport region includes a hole blocking layer, an electron transport layer, an electron injection layer, a buffer layer, or any combination thereof.

The term “organic layer” used herein refers to a single layer and/or a plurality of layers between the first electrode and the second electrode of the organic light-emitting device. The “organic layer” may include, in addition to an organic compound, an organometallic complex including metal.

In an embodiment, the heterocyclic compound may be a fluorescent emitter capable of emitting fluorescent light. An emission layer emitting the fluorescent light is clearly distinguished from an emission layer of the related art that emits phosphorescent light.

In an embodiment, the emission layer may further include a sensitizer that satisfies Equation 1 below, and the amount of the host may be greater than the sum of the amount of the sensitizer and the heterocyclic compound:

ΔE_{ST}≤0.3 eV Equation 1

In an embodiment, ΔE_{ST} refers to the energy difference between the lowest excited singlet (S₁) and the lowest excited triplet (T₁).

The triplet energy level and the singlet energy level are evaluated using the density functional theory (DFT) method of the Gaussian program, which is structure-optimized at the B3LYP/6-31G(d,p) level.

The sensitizer and the heterocyclic compound may satisfy Conditions 1 and 2:

T_{decay}(HC)<T_{decay}(S) Condition 1

T_{decay}(HC)<1.5 μs Condition 2

wherein, in Conditions 1 and 2,

T_{decay} (HC) is the decay time of the heterocyclic compound, and

T_{decay}(S) is the decay time of the sensitizer.

The decay time of the heterocyclic compound is calculated from a time-resolved photoluminescence (TRPL) at room temperature with respect to a 40 nanometer (nm)-thickness film (hereinafter referred to as “Film (CD)”) obtained by vacuum-codepositing the host and the heterocyclic compound included in the emission layer at the weight ratio of about 90:10 on a quartz substrate at the vacuum degree of 10⁻⁷ torr.

The decay time of the sensitizer is calculated from TRPL at room temperature with respect to a 40 nm-thickness film (hereinafter referred to as “Film (S)”) obtained by vacuum-codepositing the host and the sensitizer at the weight ratio of about 90:10 on a quartz substrate at the vacuum degree of 10⁻⁷ torr.

In general, it is known that since triplet excitons stay long in an excited state, they influence the decrease in the lifespan of organic light-emitting devices. While not wishing to be bound by theory, it is understood that, due to the use of the heterocyclic compound, the time during which the sensitizer stays in the triplet excitons is reduced, and thus, the lifespan of an organic light-emitting device including the same may be prolonged.

In an embodiment, the more triplet excitons the sensitizer has, the more excess energy is accumulated in the sensitizer, resulting in more hot excitons. That is, the amount of triplet excitons of the sensitizer is proportional to the amount of hot excitons. The hot excitons break down various chemical bonds of a compound included in an emission layer and/or a compound existing at the interface of the emission layer and other layers to decompose the compound, and the lifespan of organic light-emitting devices may be reduced. While not wishing to be bound by theory, it is understood that, by using heterocyclic compounds, the triplet excitons of the sensitizer can be quickly converted to singlet excitons of the heterocyclic compounds, ultimately reducing the amount of hot excitons and increasing or improving the lifespan of an organic light-emitting device.

In an embodiment, “hot excitons” may be generated or increased by exciton-exciton annihilation due to an increase in the density of excitons in an emission layer, exciton-charge annihilation due to the charge imbalance in an emission layer, and/or radical ion pairs due to the delivery of electrons between a host and a heterocyclic compound.

In order to quickly convert triplet excitons of the sensitizer to singlet excitons of the heterocyclic compound, Condition 1 should be satisfied.

While not wishing to be bound by theory, it is understood that, since the heterocyclic compound emits fluorescent light, a high or improved color purity organic light-emitting device can be provided, and in particular, since Condition 2 is satisfied, the singlet excitons of the heterocyclic compound excited state at room temperature can be rapidly transferred, and thus, the single state of the heterocyclic compound in the excited state may not be accumulated, and the lifespan of an organic light-emitting device may be increased or improved.

In an embodiment, when Condition 3 is further satisfied, the transition from the triplet excitons of the sensitizer to the singlet excitons of the heterocyclic compound may occur more smoothly, and the lifespan of an organic light-emitting device may be further prolonged or improved:

T_{decay}(HC)/T_{decay}(S)<0.5 Condition 3

wherein, in Condition 3,

T_{decay} (HC) is the decay time of the heterocyclic compound, and

T_{decay}(S) is a decay time of the sensitizer.

In an embodiment, the organic light-emitting device may further satisfy Condition 4:

BDE(S)−T₁(S)<3.0 electronvolt (eV) Condition 4

wherein, in Condition 4,

BDE (S) is the bond dissociation energy level of the sensitizer, and

T1 (S) is the lowest excitation triplet energy level of the sensitizer.

In an embodiment, the organic light-emitting device may have the target level of lifespan by satisfying Condition 5 below:

R(Hex)/e¹⁰<15 Condition 5

wherein, in Condition 5,

R (Hex) is the production rate of hot excitons.

In an embodiment, R (Hex) was subjected to the photochemical stability of the organic light-emitting device (photochemical stability), and then calculated through the Gaussian 09 program according to Equation C below,

R(Hex)=a×T_{decay}(S)×e^{-(BDE(S)-T}^{₁}^{(S)}) Equation C

wherein, in Equation C,

a is a constant,

T_{decay}(S) is a decay time of the sensitizer,

BDE (S) is the bond dissociation energy level of the sensitizer, and

T₁ (S) is the lowest excitation triplet energy level of the sensitizer.

The hot-exciton production rate is estimated to be proportional to (decay time)×e-^{(BDE-T1)}, and in order to obtain the target level of the lifespan of the organic light-emitting device, (hot-exciton production rate)/e¹⁰ should be less than about 15, for example, less than about 12, less than about 10, less than about 8, less than about 6, less than about 4, less than about 2, or less than about 1.

The degradation analysis (PCS) of organic light-emitting devices was calculated according to the following Equation P:

PCS(%)=I₂/I₁×100 Equation P

wherein, in Equation P,

I₁, with respect to a film formed by depositing a compound of which PCS is to be measured, is a maximum light intensity obtained from the PL spectrum which is evaluated at room temperature under Argon (Ar) atmosphere where ambient air is blocked immediately after the formation of the film by using a He—Cd laser (excitation wavelength=325 nanometers, laser power density)=100 mW/cm²) of KIMMON-KOHA Inc., and

I₂, with respect to a film formed by depositing a compound of which PCS is to be measured, is a maximum light intensity obtained from the PL spectrum which is evaluated at room temperature under Ar atmosphere where ambient air is blocked, by exposing the film to light of the He—Cd laser (excitation wavelength=325 nanometers and laser power density=100 milliwatts per square centimeter (mW/cm²)) of KIMMON-KOHA Inc., which is a pumping laser which has been used to evaluate 11, for 3 hours, and then, using He—Cd laser of KIMMON-KOHA Inc. (excitation wavelength=325 nanometers). In the case of the sensitizer, reverse intersystem crossing (RISC) and/or intersystem crossing (ISC) actively occur, which allows excitons generated at the host to be delivered to the heterocyclic compound.

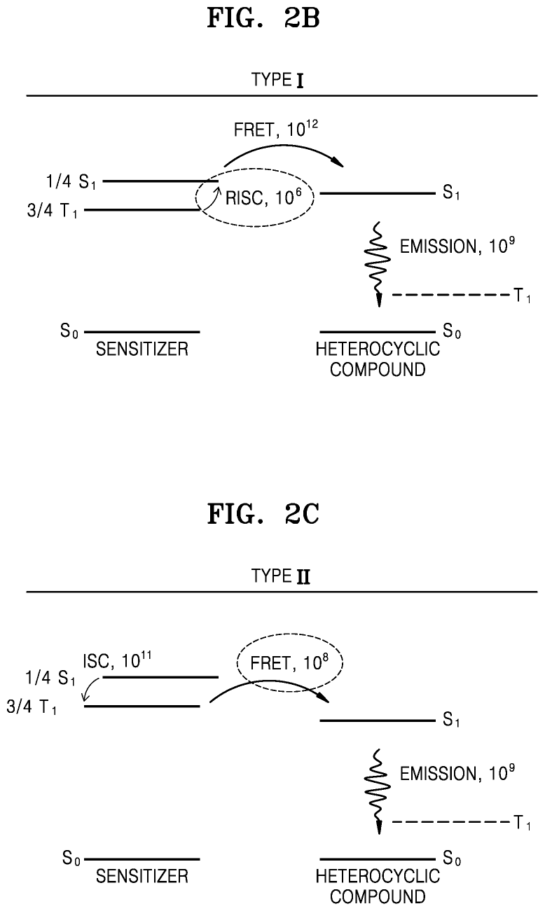

The general energy transfer of an organic light-emitting device (type I) according to an embodiment will be described with reference to FIG. 2A.

Singlet and triplet excitons are formed at the host in the emission layer, and the singlet and triplet excitons formed at the host are transferred to the sensitizer and then to the heterocyclic compound through Förster energy transfer (FRET). At this time, in order to embody the high or improved efficiency and long or improved lifespan of the organic light-emitting device, it is desirable to control the hot excitons generated in the emission layer, which may require optimization of energy transfer.

The general energy transfer of an organic light-emitting device (type I) according to an embodiment will be described with reference to FIG. 2B. This is the case when the sensitizer is a thermally activated delayed fluorescence (TADF) emitter satisfying the condition of ΔE_{ST}≤0.3 eV.

The singlet excitons formed at the host, which is about 25% of the total excitons, are transferred to the sensitizer through FRET, and the energy of triplet excitons formed at the host, which is about 75% of the total excitons, is transferred to the singlet and triplet of the sensitizer, among which the energy delivered to triplet is subjected to RISC into singlet, and then, the singlet energy of the sensitizer is transferred to the heterocyclic compound through FRET.

The general energy transfer of an organic light-emitting device (type II) according to an embodiment will be described with reference to FIG. 2C. In an embodiment, the sensitizer is an organic metallic compound including at least one metal of Period 1 transition metal, Period 2 transition metal, or Period 3 transition metal.

The triplet excitons formed at the host, which is about 75% of the total excitons, are transferred to the sensitizer through Dexter energy transfer, and the energy of singlet excitons formed at the host, which is about 25% of the total excitons, is transferred to the singlet and triplet of the sensitizer, among which the energy delivered to singlet is subjected to ISC into triplet, and then, the triplet energy of the sensitizer is transferred to the heterocyclic compound through FRET.

While not wishing to be bound by theory, it is understood that by transferring all the singlet excitons and triplet excitons generated in the emission layer to the heterocyclic compound, an organic light-emitting device having improved efficiency can be obtained. In an embodiment, since an organic light-emitting device can be obtained with significantly reduced energy loss, the lifespan characteristics of the organic light-emitting device can be improved.

The amount of the sensitizer in the emission layer may be from about 5 weight percent (wt %) to about 50 wt % with respect to the total weight of the emission layer, for example, from about 5 wt % to about 40 wt %, from about 5 wt % to about 30 wt %, from about 5 wt % to about 20 wt %, from about 5 wt % to about 10 wt %, from about 10 wt % to about 50 wt %, from about 15 wt % to about 50 wt %, from about 20 wt % to about 50 wt %, from about 25 wt % to about 50 wt %, from about 30 wt % to about 50 wt %, from about 35 wt % to about 50 wt %, from about 40 wt % to about 50 wt %, or from about 45 wt % to about 50 wt % with respect to the total weight of the emission layer. While not wishing to be bound by theory, it is understood that within these ranges, it is possible to achieve effective energy transfer in the emission layer, and accordingly, an organic light-emitting device having high or improved efficiency and long or improved lifespan can be obtained.

In an embodiment, the host, the heterocyclic compound, and the sensitizer may satisfy Condition 6:

T₁(H)≥T₁(S)≥S₁(HC) Condition 6

wherein, in Condition 6,

T₁(H) is the lowest excited triplet energy level of the host,

S₁(HC) is the lowest excitation singlet energy level of the heterocyclic compound, and

T₁(S) is the lowest excitation triplet energy level of the sensitizer.

While not wishing to be bound by theory, it is understood that when the host, the heterocyclic compound, and the sensitizer further satisfy condition 6, triplet excitons may be effectively transferred from the host to the heterocyclic compound, thereby obtaining an organic light-emitting device with improved efficiency.

The emission layer may include the host, the heterocyclic compound, and the sensitizer. That is, the emission layer may not further include materials other than the host, the heterocyclic compound, and the sensitizer.

In an embodiment, the emission layer may further include a light-emitting dopant, and in this case, the amount of the host may be greater than the total amount of the light emitting dopant and the heterocyclic compound represented by Formula 1-1 or 1-2. The light-emitting dopant may include a light-emitting dopant having S₁ and T₁ energy levels suitable for receiving energy from the excitation S₁ energy level of the heterocyclic compound. In an embodiment, the heterocyclic compound may act as a sensitizer that transfers energy. In an embodiment, the heterocyclic compound and the light-emitting dopant may satisfy the same relationship of the sensitizer and the heterocyclic compound described above.

The emission layer may emit deep blue light. For example, the blue light refers to light having the wavelength range of about 380 nm to about 490 nm.

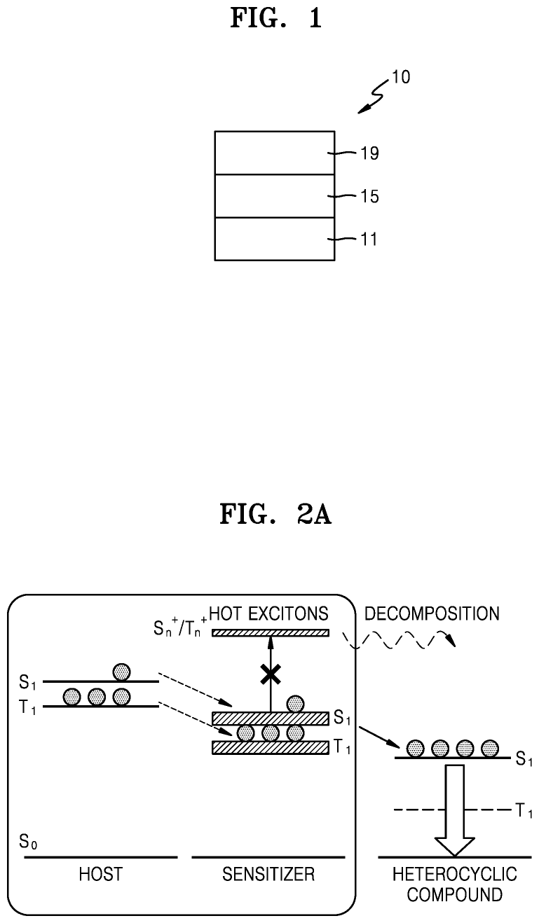

FIG. 1 is a schematic cross-sectional view of an organic light-emitting device 10 according to an embodiment. Hereinafter, the structure of an organic light-emitting device according to an embodiment of the present disclosure and a method of manufacturing an organic light-emitting device according to an embodiment of the present disclosure will be described in connection with FIG. 1. The organic light-emitting device 10 includes a first electrode 11, an organic layer 15, and a second electrode 19, which are sequentially stacked.

A substrate may be additionally located under the first electrode 11 or above the second electrode 19. For use as the substrate, any suitable substrate that is used in organic light-emitting devices available in the art may be used, and the substrate may be a glass substrate or a transparent plastic substrate, each having excellent or suitable mechanical strength, thermal stability, transparency, surface smoothness, ease of handling, and water resistance.

In an embodiment, the first electrode 11 may be formed by depositing or sputtering a material for forming the first electrode 11 on the substrate. The first electrode 11 may be an anode. The material for forming the first electrode 11 may be materials with a high work function to facilitate hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In an embodiment, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 is located on the first electrode 11.

The organic layer 15 may include a hole transport region, an emission layer, and an electron transport region.

The hole transport region may be between the first electrode 11 and the emission layer.

The hole transport region may include at least one of a hole injection layer, a hole transport layer, an electron blocking layer, or a buffer layer.

The hole transport region may include only either a hole injection layer or a hole transport layer. In an embodiment, the hole transport region may have a hole injection layer/hole transport layer structure or a hole injection layer/hole transport layer/electron blocking layer structure, wherein, for each structure, each layer is sequentially stacked in this stated order from the first electrode 11.

When the hole transport region may include a hole injection layer (HIL), the hole injection layer may be formed on the first electrode 11 by using one or more suitable methods, for example, vacuum deposition, spin coating, casting, and/or Langmuir-Blodgett (LB) deposition.

In an embodiment, when a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., for example, about 100° C. to about 400° C., about 100° C. to about 300° C., or about 100° C. to about 200° C.; a vacuum pressure of about 10⁻⁸ torr to about 10⁻³ torr, for example, about 10⁻⁸ torr to about 10⁻⁶ torr, about 10⁻⁸ torr to about 10⁻⁵ torr, or about 10⁻⁸ torr to about 10⁻⁴ torr; and a deposition rate of about 0.01 angstrom per second (Å/sec) to about 100 Å/sec, for example, about 0.01 Å/sec to about 80 Å/sec, about 0.01 Å/sec to about 60 Å/sec, about 0.01 Å/sec to about 40 Å/sec, about 0.01 Å/sec to about 20 Å/sec, about 0.01 Å/sec to about 10 Å/sec, or about 0.01 Å/sec to about 5 Å/sec. However, the deposition conditions are not limited thereto.

In an embodiment, when the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, for example, about 2,000 rpm to about 4,000 rpm, about 2,000 rpm to about 3,000 rpm, or about 2,000 rpm to about 2,500 rpm, and a temperature at which a heat treatment is performed to remove a solvent after coating may be from about 80° C. to about 200° C., for example, about 80° C. to about 180° C., about 80° C. to about 160° C., about 80° C. to about 140° C., about 80° C. to about 120° C., or about 80° C. to about 100° C. However, the coating conditions are not limited thereto.

The conditions for forming the hole-transporting layer and the electron-blocking layer may be the same as the conditions for forming the hole injection layer.

The hole transport region may include at least one of m-MTDATA, TDATA, 2-TNATA, NPB, β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201, and a compound represented by Formula 202:

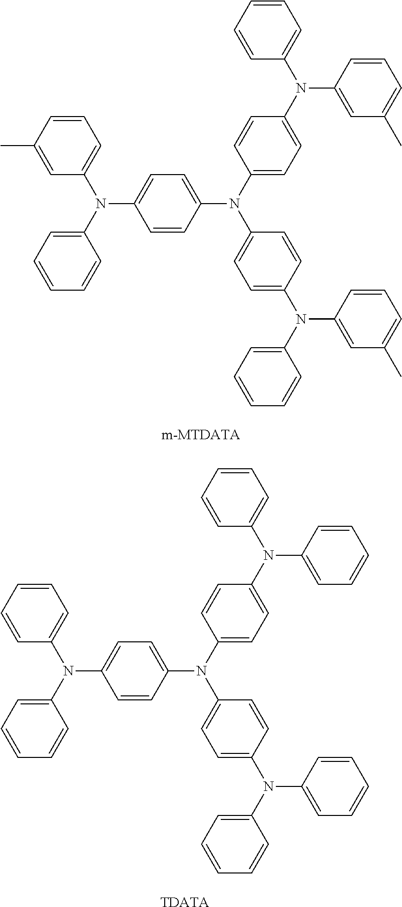

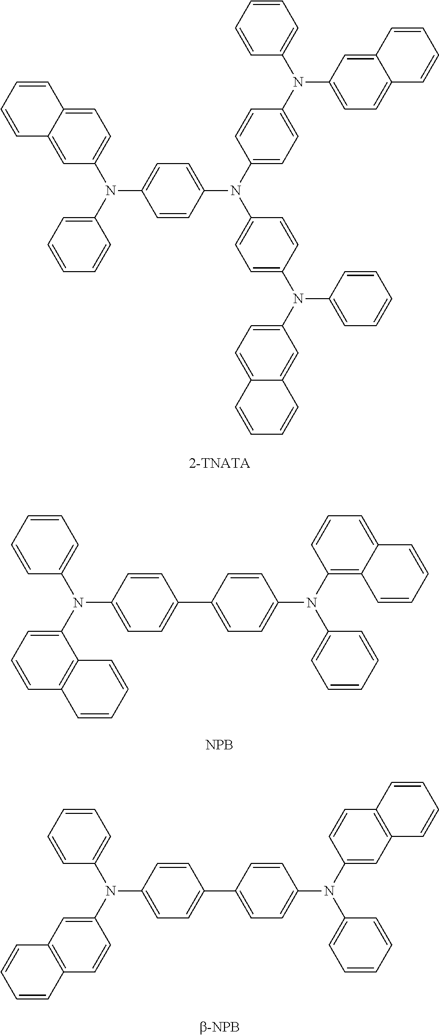

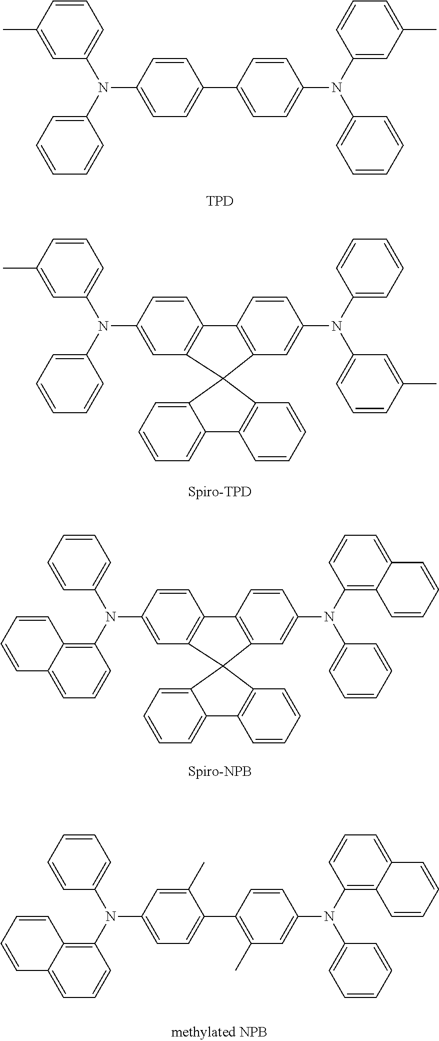

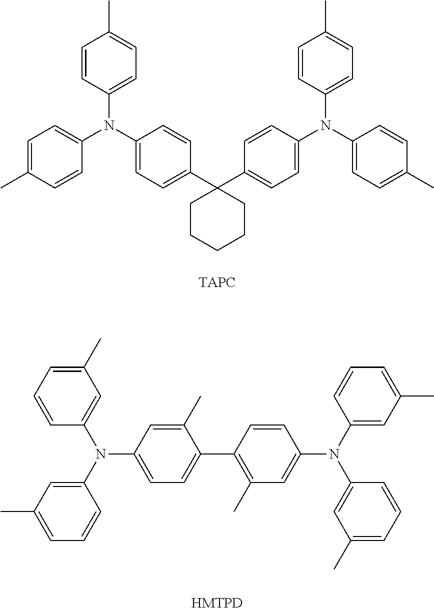

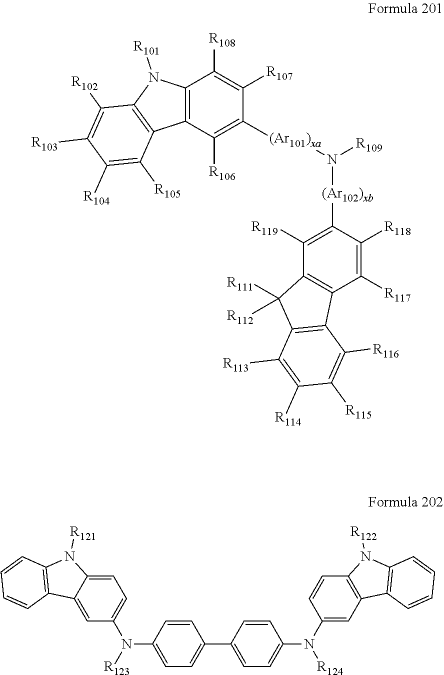

Wherein Ar₁₀₁ and Ar₁₀₂ in Formula 201 may each independently be:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₃-C₁₀ cycloalkenyl group, a C₁-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₁-C₆₀ heteroalkylaryl group, a monovalent non-aromatic condensed polycyclic group, or a monovalent non-aromatic condensed heteropolycyclic group.

xa and xb in Formula 201 may each independently be an integer from 0 to 5, or 0, 1, or 2. For example, xa may be 1 and xb may be 0, but xa and xb are not limited thereto.

R₁₀₁ to R₁₀₈, R₁₁₁ to R₁₁₉ and R₁₂₁ to R₁₂₄ in Formulae 201 and 202 may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.), a C₁-C₁₀ alkoxy group (for example, a methoxy group, an ethoxy group, a propoxy group, a butoxy group, a pentoxy group, etc.) or a C₁-C₁₀ alkylthio group (for example, a thiomethyl group, an thioethyl group, a thiopropyl group, a thiobutyl group, thiopentyl group, a thiohexyl group, etc.);

a C₁-C₁₀ alkyl group, a C₁-C₁₀ alkylthio group, or a C₁-C₁₀ alkoxy group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof or a phosphoric acid group or a salt thereof;

a phenyl group, a naphthyl group, an anthracenyl group, a fluorenyl group, or a pyrenyl group; or

a phenyl group, a naphthyl group, an anthracenyl group, a fluorenyl group, and a pyrenyl group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkylthio group, or a C₁-C₁₀ alkoxy group,

but embodiments of the present disclosure are not limited thereto.

R₁₀₉ in Formula 201 may be:

a phenyl group, a naphthyl group, an anthracenyl group, or a pyridinyl group; or

a phenyl group, a naphthyl group, an anthracenyl group, and a pyridinyl group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a naphthyl group, an anthracenyl group, or a pyridinyl group.

According to an embodiment, the compound represented by Formula 201 may be represented by Formula 201A, but embodiments of the present disclosure are not limited thereto:

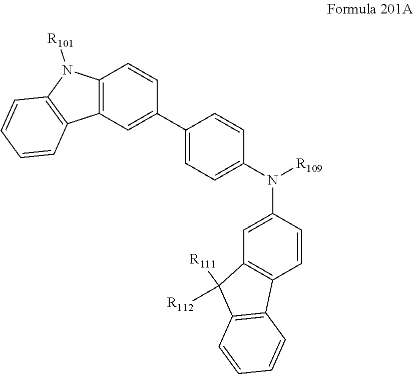

Wherein R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but are not limited thereto:

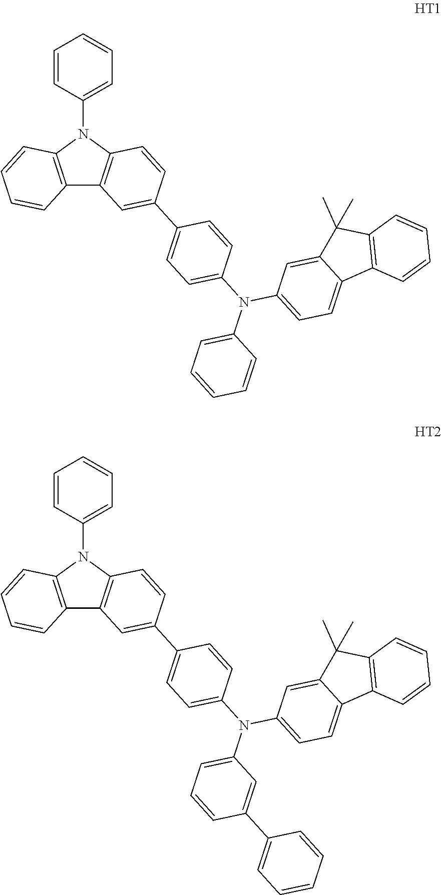 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21]

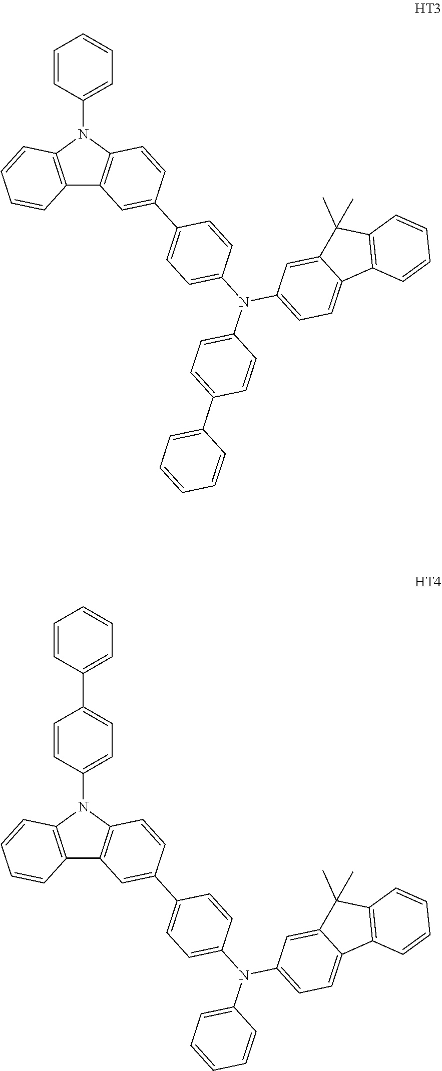 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

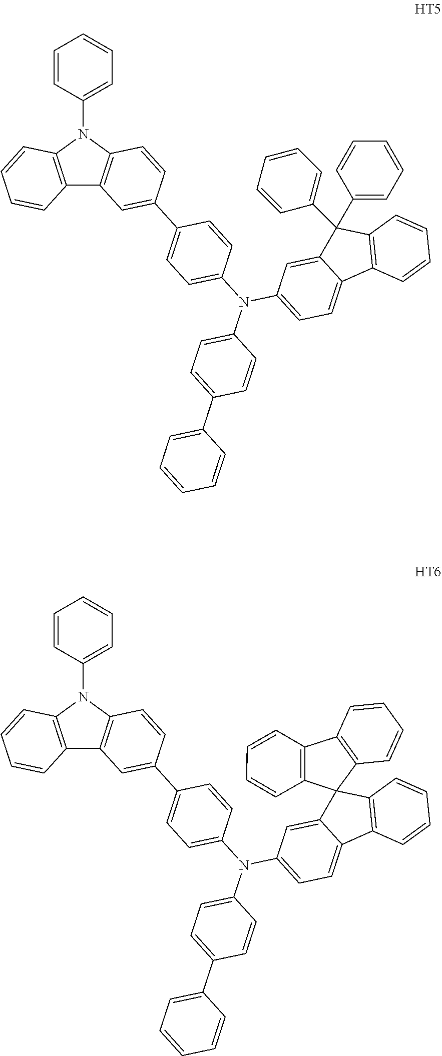 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

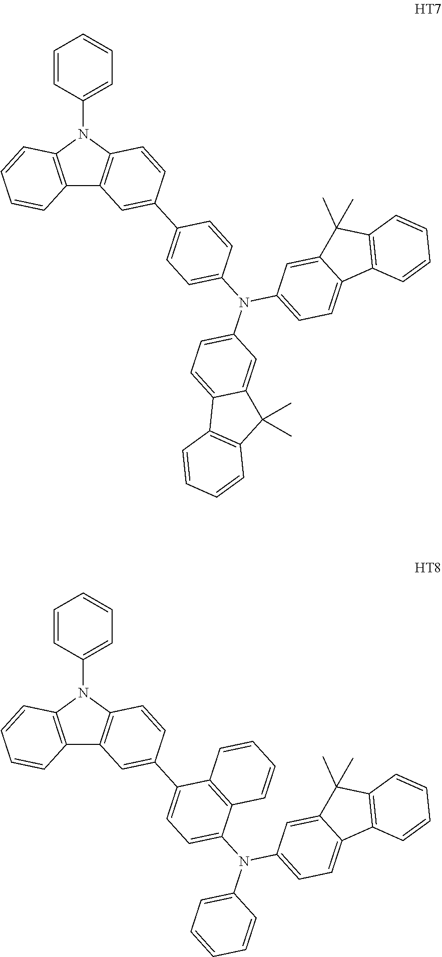 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

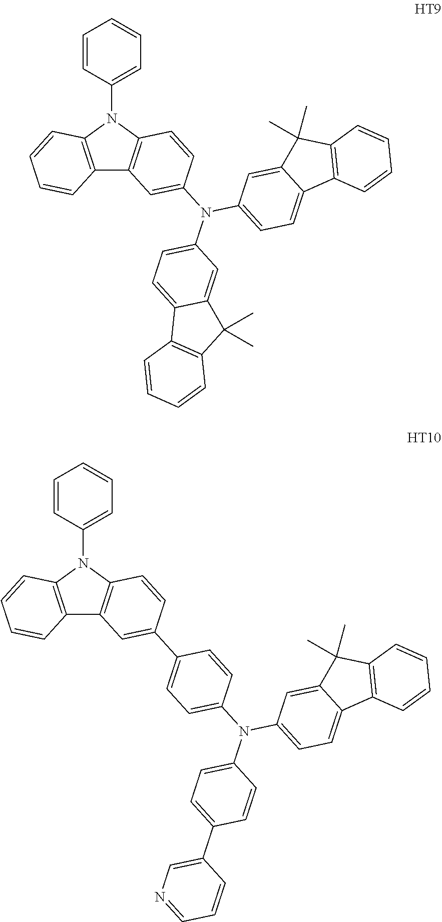 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

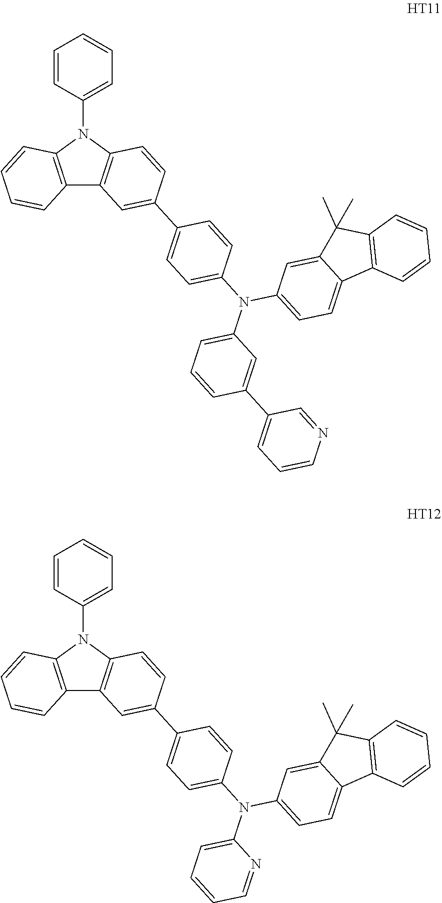 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

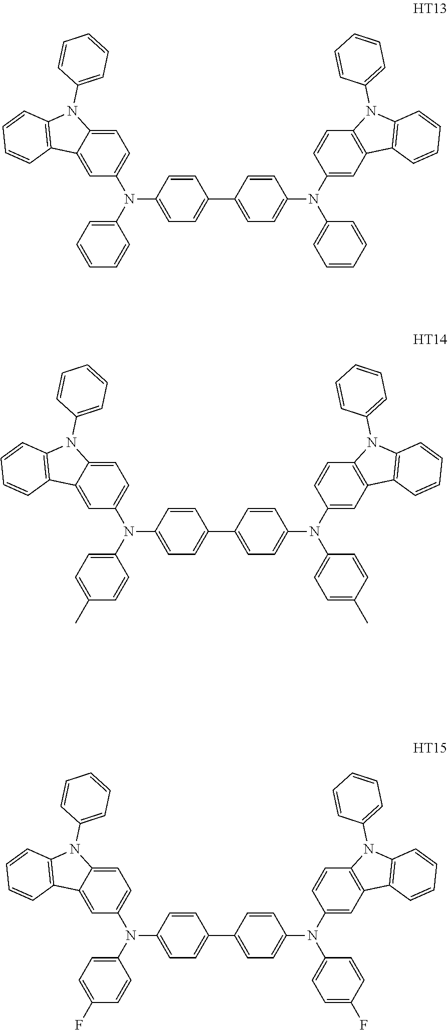 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

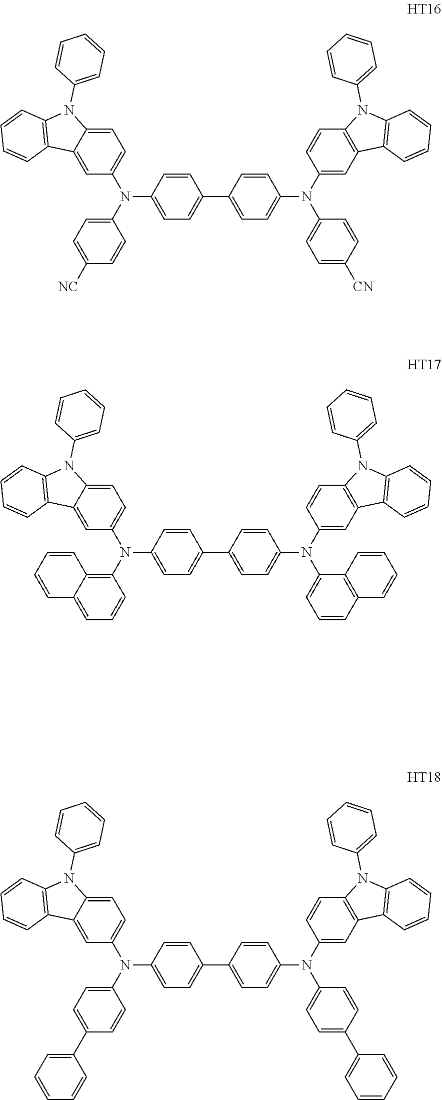 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

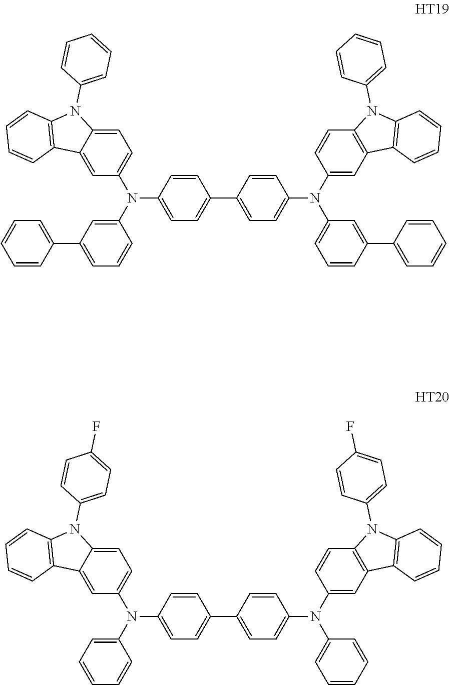 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in the range of about 100 Å to about 10,000 Å, for example, about 100 Å to about 1,000 Å, about 100 Å to about 800 Å, about 200 Å to about 600 Å, or about 300 Å to about 400 Å. When the hole transport region includes all of a hole injection layer and a hole transport layer, a thickness of the hole injection layer may be in a range of about 100 Å to about 10,000 Å, for example, about 100 Å to about 1,000 Å, about 100 Å to about 2,000 Å, about 200 Å to about 3,000 Å, about 300 Å to about 4,000 Å, about 400 Å to about 5,000 Å, about 500 Å to about 6,000 Å, about 600 Å to about 7,000 Å, or about 800 Å to about 9,000 Å, and a thickness of the hole transport layer may be in a range of about 50 Å to about 2,000 Å, for example, about 100 Å to about 1,500 Å, about 100 Å to about 1500 Å, about 400 Å to about 1,000 Å, or about 500 Å to about 900 Å. While not wishing to be bound by theory, it is understood that when the thicknesses of the hole-transporting region, the hole injection layer and the hole-transporting layer are within these ranges, satisfactory or improved hole-transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole-transporting region.

The charge-generation material may be, for example, a p-dopant. The p-dopant may be one of a quinone derivative, a metal oxide, or a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 or Compound HT-D2 below, but are not limited thereto.

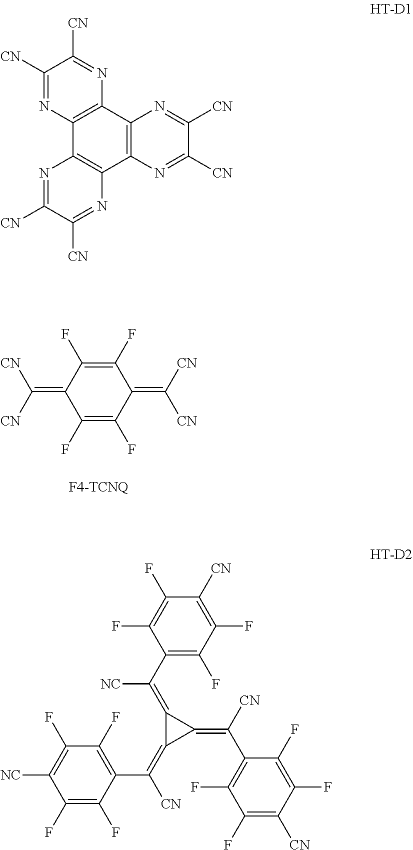

The hole transport region may include a buffer layer.

In an embodiment, the buffer layer may compensate for an optical resonance distance according to a wavelength of light emitted from the emission layer, and thus, efficiency of a formed organic light-emitting device may be improved.

The hole transport region may further include an electron blocking layer. The electron blocking layer may include a suitable material available in the art, for example, mCP, but embodiments of the present disclosure are not limited thereto.

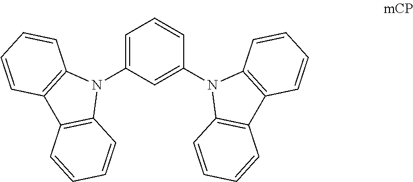 [SMILES: c1cc(-n2c3ccccc3c3ccccc32)cc(-n2c3ccccc3c3ccccc32)c1]

In an embodiment, an emission layer (EML) may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a material that is used to form the emission layer.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The emission layer may include a host and a dopant, the dopant may include a heterocyclic compound represented by Formula 1-1 or 1-2, and the heterocyclic compound is the same as described in the present specification.

The host may include at least one of TPBi, TBADN, ADN (also referred to as “DNA”), CBP, CDBP, TCP, mCP, or Compound H50 to Compound H52:

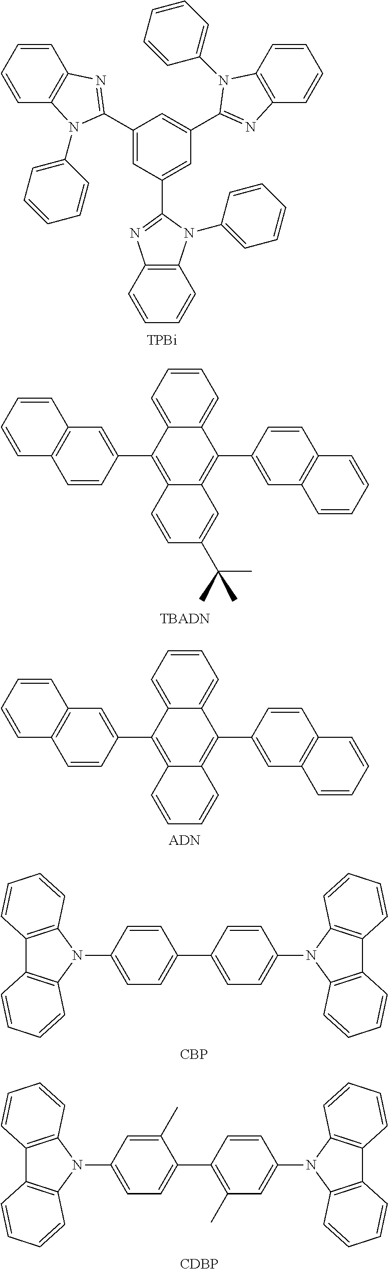

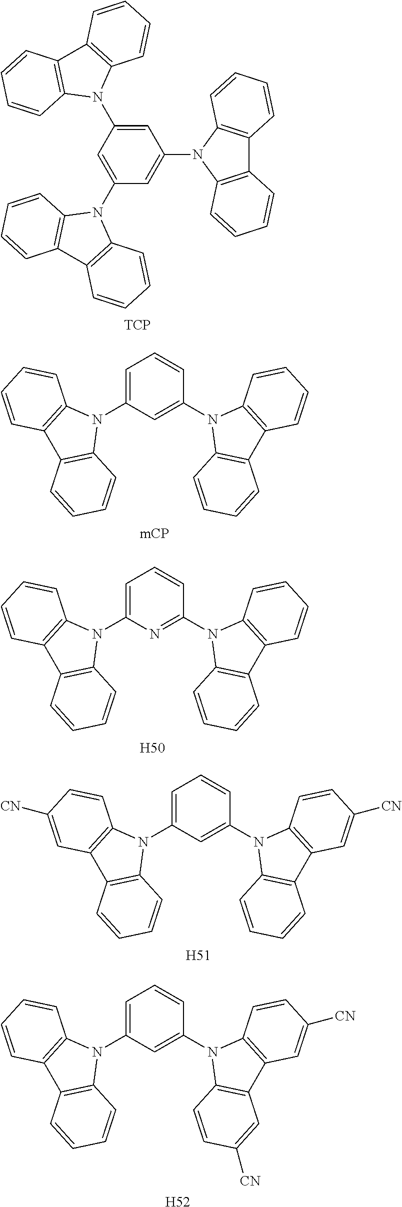

In an embodiment, the host may further include a compound represented by Formula 301:

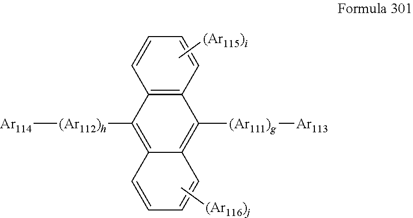

wherein Ar₁₁₁ and Ar₁₁₂ in Formula 301 may each independently be:

a phenylene group, a naphthylene group, a phenanthrenylene group, or a pyrenylene group; or

a phenylene group, a naphthylene group, a phenanthrenylene group, or a pyrenylene group, each independently substituted with at least one of a phenyl group, a naphthyl group, or an anthracenyl group.

Ar₁₁₃ to Ar₁₁₆ in Formula 301 may each independently be:

a C₁-C₁₀ alkyl group, a phenyl group, a naphthyl group, a phenanthrenyl group, or a pyrenyl group; or

a phenyl group, a naphthyl group, a phenanthrenyl group, or a pyrenyl group, each independently substituted with at least one of a phenyl group, a naphthyl group, or an anthracenyl group.

g, h, i, and j in Formula 301 may each independently be an integer from 0 to 4, and may be, for example, 0, 1, or 2.

Ar₁₁₃ to Ar₁₁₆ in Formula 301 may each independently be:

a C₁-C₁₀ alkyl group, substituted with at least one of a phenyl group, a naphthyl group, or an anthracenyl group;

a phenyl group, a naphthyl group, an anthracenyl group, a pyrenyl, a phenanthrenyl group, or a fluorenyl group;

a phenyl group, a naphthyl group, an anthracenyl group, a pyrenyl group, a phenanthrenyl group, or a fluorenyl group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a phenyl group, a naphthyl group, an anthracenyl group, a pyrenyl group, a phenanthrenyl group, or a fluorenyl group; or

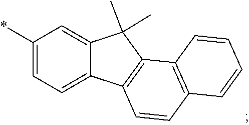 [SMILES: *c1ccc2c(c1)C(C)(C)c1c-2ccc2ccccc12]

but embodiments of the present disclosure are not limited thereto.

In an embodiment, the host may include a compound represented by Formula 302 below:

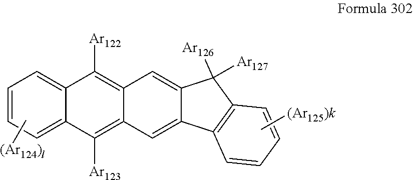

wherein Ar₁₂₂ to Ar₁₂₅ in Formula 302 are the same as described in detail in connection with Ar₁₁₃ in Formula 301.

Ar₁₂₆ and Ar₁₂₇ in Formula 302 may each independently be a C₁-C₁₀ alkyl group (for example, a methyl group, an ethyl group, or a propyl group).

k and l in Formula 302 may each independently be an integer from 0 to 4. For example, k and l may be 0, 1, or 2.

When the emission layer includes a host and a dopant, an amount of the dopant may be in a range of about 0.01 parts by weight to about 15 parts by weight based on 100 parts by weight of the host, for example, about 0.01 parts by weight to about 12 parts by weight, about 0.01 parts by weight to about 10 parts by weight, about 0.01 parts by weight to about 8 parts by weight, about 0.01 parts by weight to about 6 parts by weight, about 0.01 parts by weight to about 4 parts by weight, or about 0.01 parts by weight to about 2 parts by weight based on 100 parts by weight of the host, but embodiments of the present disclosure are not limited thereto.

A thickness of the emission layer may be in a range of about 100 Å to about 1,000 Å, for example, about 100 Å to about 800 Å, about 200 Å to about 600 Å, or about 300 Å to about 400 Å. While not wishing to be bound by theory, it is understood that when the thickness of the emission layer is within these ranges, excellent or improved light-emission characteristics may be obtained without a substantial increase in driving voltage.

Then, an electron transport region may be located on the emission layer.

The electron transport region may include at least one of a hole-blocking layer, an electron transport layer, or an electron injection layer.

For example, the electron transport region may have a hole blocking layer/electron transport layer/electron injection layer structure or an electron transport layer/electron injection layer structure, and the structure of the electron transport region is not limited thereto. The electron-transporting layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP and Bphen, but embodiments of the present disclosure are not limited thereto.

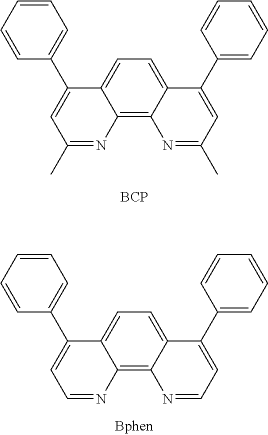

A thickness of the hole-blocking layer may be in a range of about 20 Å to about 1,000 Å, for example, about 10 Å to about 100 Å, about 20 Å to about 200 Å, about 30 Å to about 300 Å, about 40 Å to about 400 Å, about 50 Å to about 500 Å, about 60 Å to about 600 Å, about 70 Å to about 700 Å, about 80 Å to about 800 Å, about 90 Å to about 900 Å, or about 100 Å to about 1000 Å. While not wishing to be bound by theory, it is understood that when the thickness of the hole-blocking layer is within these ranges, excellent or improved hole-blocking characteristics can be obtained without a substantial increase in driving voltage.

The electron transport layer may further include at least one of BCP, Bphen, Alq3, BAlq, TAZ, or NTAZ.

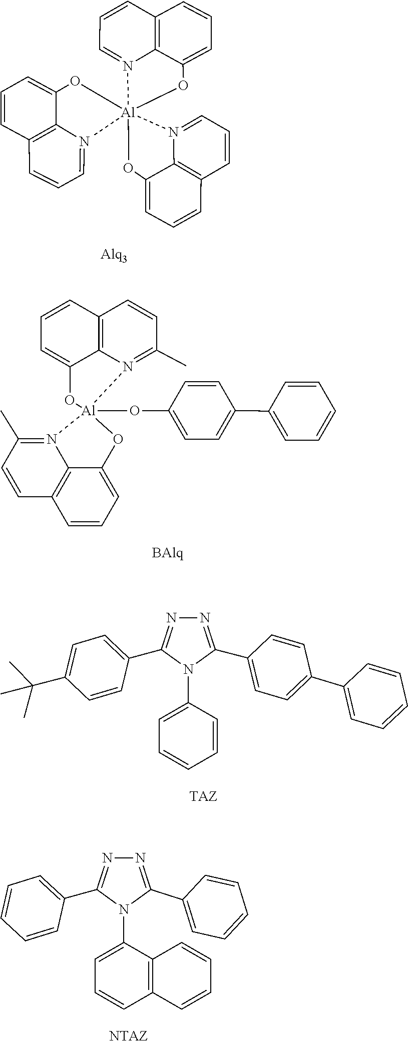

In an embodiment, the electron transport layer may include at least one of ET1 to ET25, but are not limited thereto:

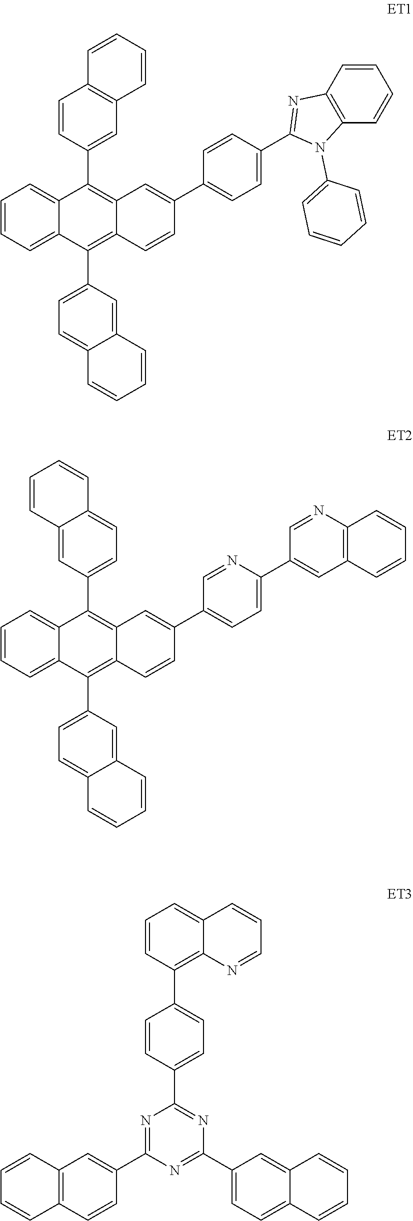 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

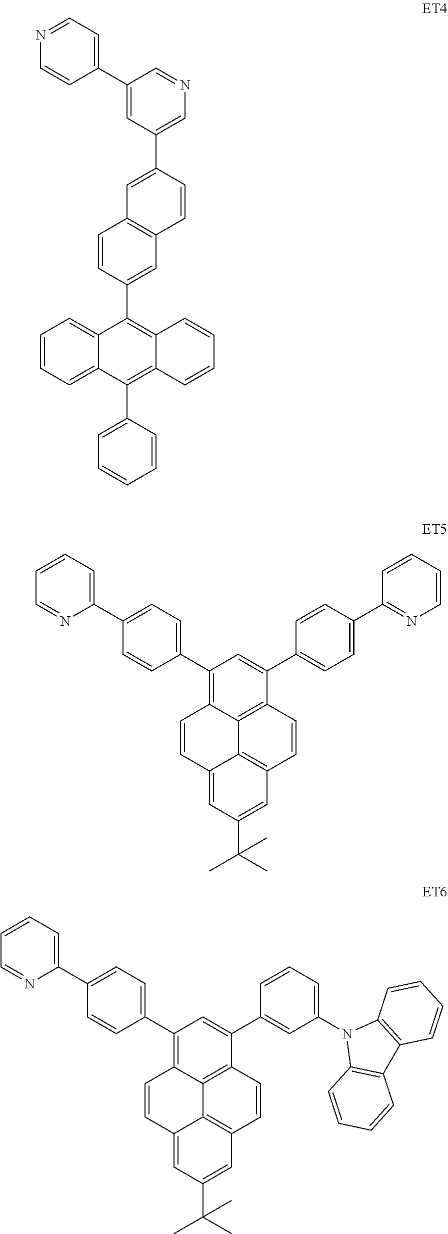 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

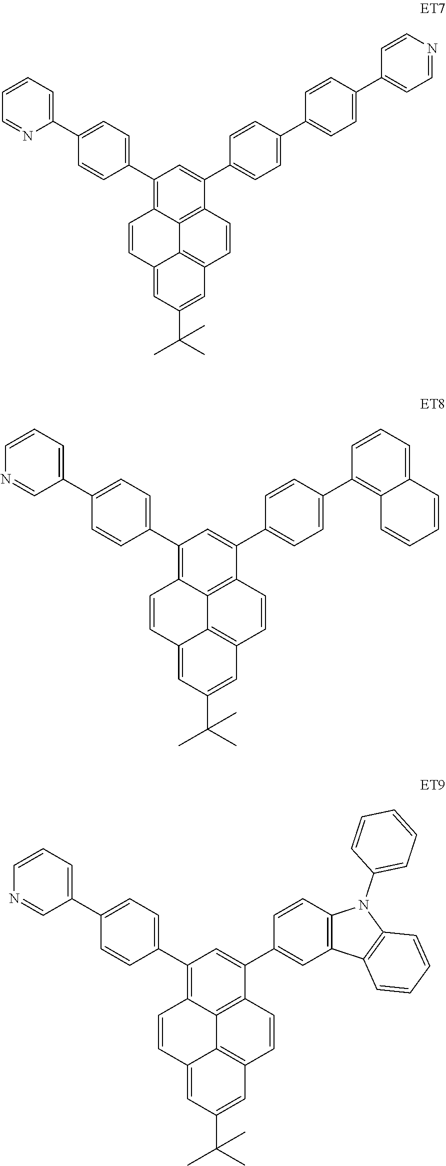 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34]

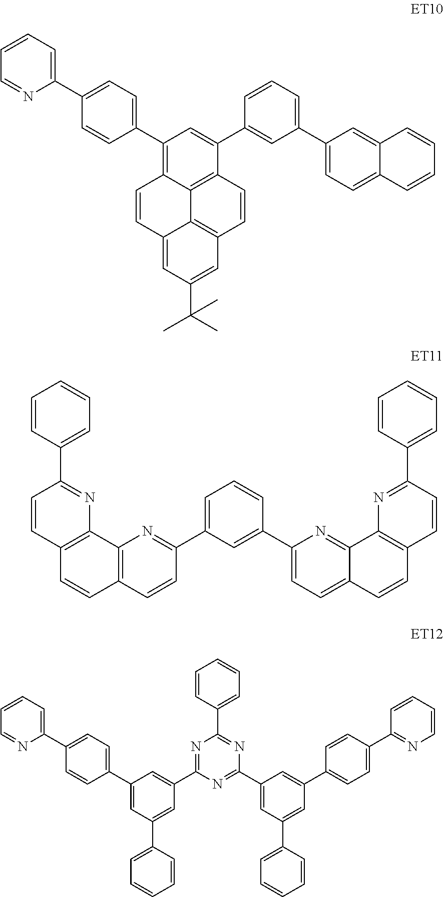 [SMILES: CC(C)(C)c1cc2ccc3c(-c4ccc(-c5ccccn5)cc4)cc(-c4cccc(-c5ccc6ccccc6c5)c4)c4ccc(c1)c2c34 ; c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1]

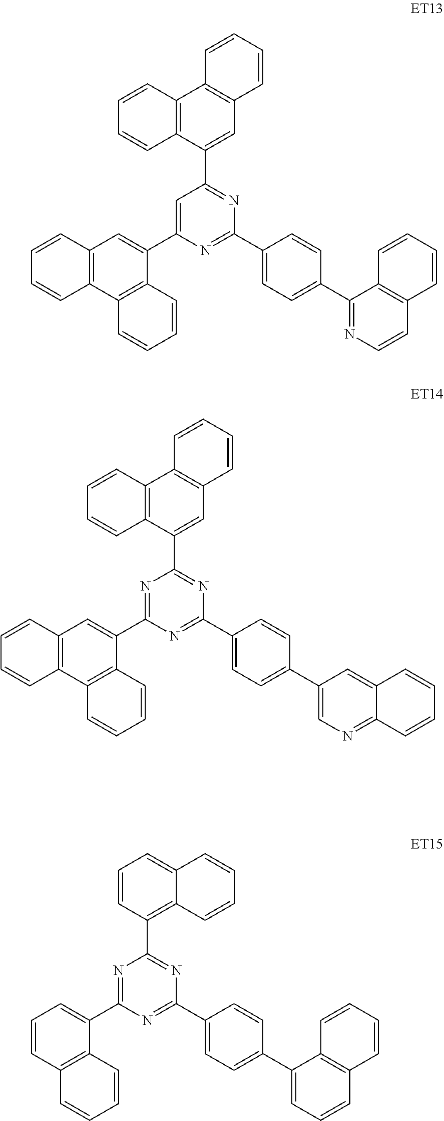 [SMILES: c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc2c(-c3ccc(-c4nc(-c5cccc6ccccc56)nc(-c5cccc6ccccc56)n4)cc3)cccc2c1]

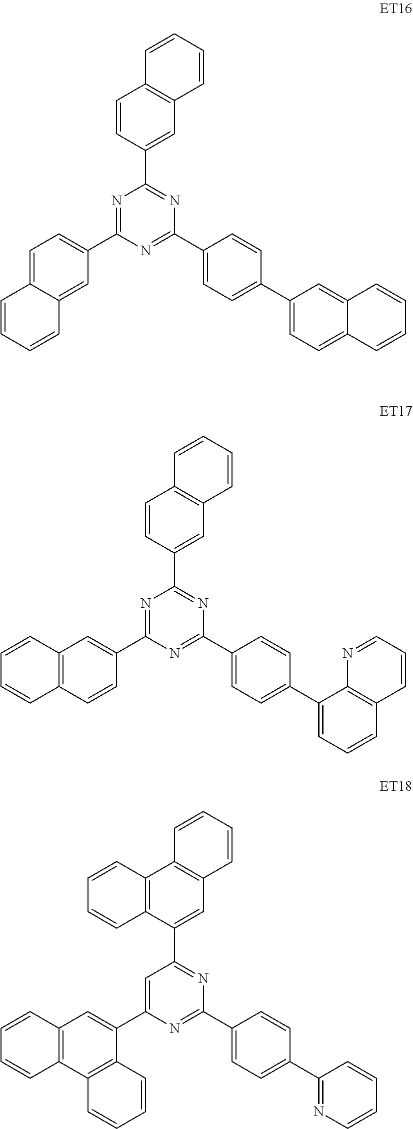 [SMILES: c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4cc5ccccc5c5ccccc45)cc(-c4cc5ccccc5c5ccccc45)n3)cc2)nc1]

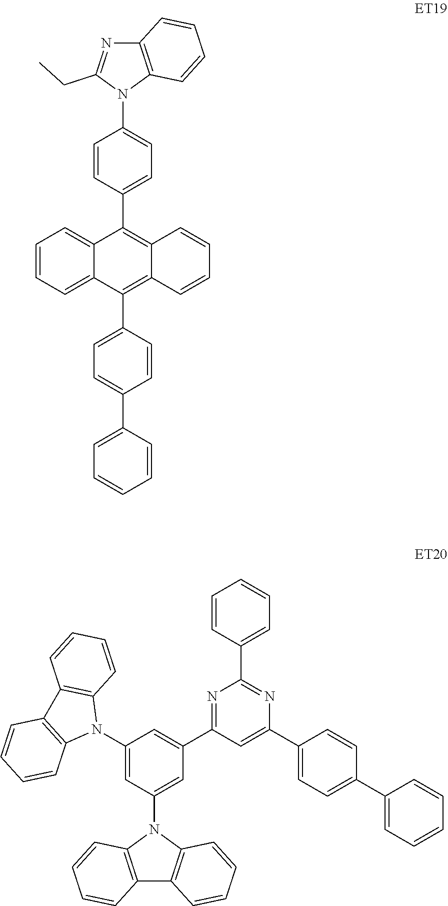 [SMILES: CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

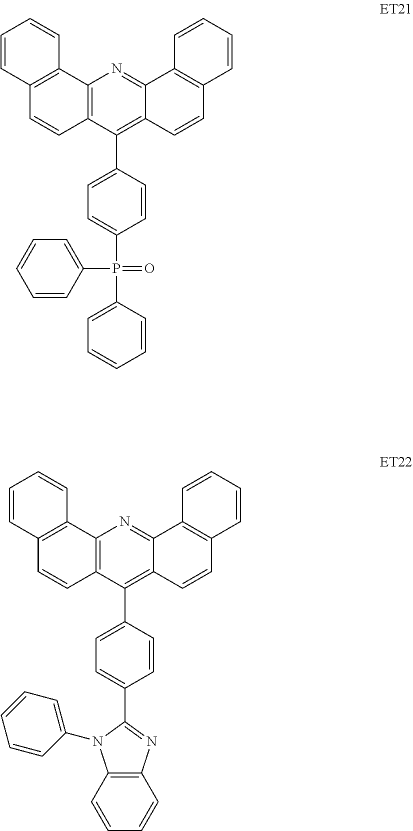 [SMILES: O=P(c1ccccc1)(c1ccccc1)c1ccc(-c2c3ccc4ccccc4c3nc3c2ccc2ccccc23)cc1 ; c1ccc(-n2c(-c3ccc(-c4c5ccc6ccccc6c5nc5c4ccc4ccccc45)cc3)nc3ccccc32)cc1]

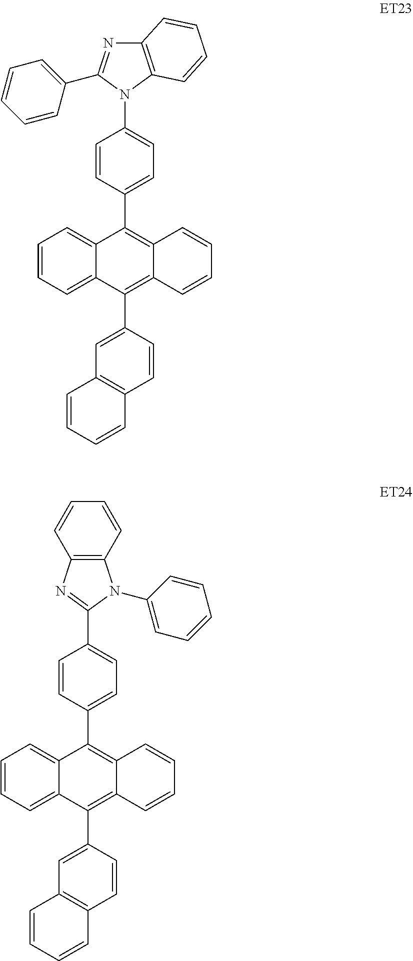 [SMILES: c1ccc(-c2nc3ccccc3n2-c2ccc(-c3c4ccccc4c(-c4ccc5ccccc5c4)c4ccccc34)cc2)cc1 ; c1ccc(-n2c(-c3ccc(-c4c5ccccc5c(-c5ccc6ccccc6c5)c5ccccc45)cc3)nc3ccccc32)cc1]

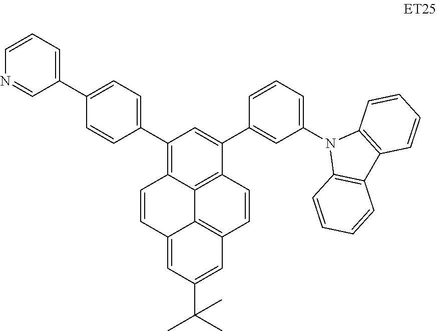 [SMILES: CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron-transporting layer may be in the range of about 100 Å to about 1,000 Å, for example, about 150 Å to about 500 Å, about 200 Å to about 600 Å, about 250 Å to about 700 Å, about 300 Å to about 800 Å, about 350 Å to about 900 Å, or about 400 Å to about 1000 Å. While not wishing to be bound by theory, it is understood that when the thickness of the electron-transporting layer is within the range described above, the electron-transporting layer may have satisfactory or improved electron-transporting characteristics without a substantial increase in driving voltage.

The electron-transporting layer may include a metal-containing material in addition to the material as described above.

The metal-containing material may include a Li complex. The Li complex may include, for example, Compound ET-D1 (lithium quinolate, LiQ) or ET-D2:

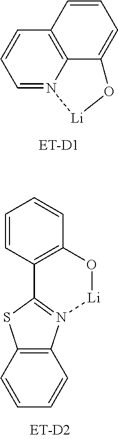

The electron transport region may include an electron injection layer (EIL) that promotes the flow of electrons from the second electrode 19 thereinto.

The electron injection layer may include at least one of LiF, NaCl, CsF, Li₂O, or BaO.

A thickness of the electron injection layer may be in a range of about 1 Å to about 100 Å, and, for example, about 3 Å to about 90 Å, about 6 Å to about 80 Å, about 9 Å to about 70 Å, about 12 Å to about 60 Å, about 15 Å to about 50 Å, about 18 Å to about 40 Å, or about 20 Å to about 30 Å. While not wishing to be bound by theory, it is understood that when the thickness of the electron injection layer is within the range described above, the electron injection layer may have satisfactory or improved electron injection characteristics without a substantial increase in driving voltage.

The second electrode 19 is located on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (AI), aluminum-lithium (Al—Li), calcium (Ca), magnesium-indium (Mg—In), or magnesium-silver (Mg—Ag) may be used as the material for forming the second electrode 19. In an embodiment, to manufacture a top-emission type light-emitting device, a transmissive electrode formed using ITO or IZO may be used as the second electrode 19.

Hereinbefore, the organic light-emitting device has been described with reference to FIG. 1, but embodiments of the present disclosure are not limited thereto.

### Explanation of Terms

In the present specification, the C₅-C₃₀ carbocyclic group refers to an aromatic or non-aromatic mono or polycyclic group having 1 to 30 carbon atoms. The C₁-C₃₀ heterocyclic group refers to an aromatic or non-aromatic mono or polycyclic group having 1 to 30 carbon atoms which includes at least one heteroatom N, O, P, Si, B, Se, or S as a ring-forming atom.

The term “C₁-C₆₀ alkyl group” as used herein refers to a linear or branched aliphatic saturated hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group.

The term “C₁-C₆₀ alkylene group” as used herein refers to a divalent group having the same structure as the C₁-C₆₀ alkyl group.

The term “C₁-C₆₀ alkoxy group” used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₁-C₆₀ alkylthio group” used herein refers to a monovalent group represented by —SA₁₀₈ (wherein A₁₀₁ is the C₁-C₆₀ alkyl group), and examples thereof include a thiomethyl group, a thioethyl group, and a thioisopropyl group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom N, O, P, Si, B, Se, or S as a ring-forming atom and 1 to 10 carbon atoms, and examples thereof include a tetrahydrofuranyl group and a tetrahydrothiophenyl group. The term “C₂-C₁₀ heterocycloalkylene group” as used herein refers to a divalent group having the same structure as the C₁-C₁₀ heterocycloalkyl group.

The term C₃-C₁₀ cycloalkenyl group used herein refers to a monovalent monocyclic group that has 3 to 10 carbon atoms and at least one carbon-carbon double bond in the ring thereof and no aromaticity, and examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one hetero atom N, O, P, Si, B, Se, or S as a ring-forming atom, 2 to 10 carbon atoms, and at least one carbon-carbon double bond in its ring. Examples of the C₂-C₁₀ heterocycloalkenyl group are a 2,3-hydrofuranyl group, and a 2,3-hydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group or a benzene group, a naphthyl group, an anthracenyl group, a phenanthrenyl group, a pyrenyl group, and a chrysenyl group. For example, a “benzene group” may be a benzo group, a phenyl group, a phenylene group, or the like, and this may be understood by one of ordinary skill in the art, depending on the structure of the formula including the “benzene group”. When the C₆-C₆₀ aryl group and the C₆-C₆₀ arylene group each include two or more rings, the rings may be fused to each other.

The term “C₇-C₆₀ alkylaryl group” as used herein refers to a C₆-C₅₉ aryl group substituted with at least one C₁-C₅₄ alkyl or alkylene group.

The term “C₁-C₆₀ heteroaryl group” as used herein refers to a monovalent group having a cyclic aromatic system having at least one heteroatom N, O, P, Si, B, Se, or S as a ring-forming atom and a carbocyclic aromatic system having 1 to 60 carbon atoms, and the term “C₁-C₆₀ heteroarylene group” as used herein refers to a divalent group having a cyclic aromatic system having at least one hetero atom N, O, P, Si, B, Se, or S as a ring-forming atom and a carbocyclic aromatic system having 1 to 60 carbon atoms. Examples of the C₂-C₆₀ heteroaryl group include a pyridinyl group, a pyrimidinyl group, a pyrazinyl group, a pyridazinyl group, a triazinyl group, a quinolinyl group, and an isoquinolinyl group.

When the C₁-C₆₀ heteroaryl group and the C₁-C₆₀ heteroarylene group each include two or more rings, the rings may be fused to each other.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The “C₇-C₆₀ arylalkyl group” as used herein refers to -A₁₀₄A₁₀₅ (where A₁₀₄ is a C₁-C₅₄ alkyl group, and A₁₀₅ is a C₆-C₅₉ aryl group), and includes, for example, a cumyl group.

The “C₂-C₆₀ heteroarylalkyl group” as used herein refers to -A₁₀₆A₁₀₇ (where A₁₀₆ is a C₁-C₅₉ alkyl group, and A₁₀₇ is a C₁-C₅₉ heteroaryl group).

The term “C₁-C₆₀ heteroaryloxy group” as used herein refers to —OA₁₀₉ (wherein A₁₀₉ is the C₁-C₆ heteroaryl group), the term “C₁-C₆₀ heteroarylthio group” as used herein indicates —SA₁₁₀ (wherein A₁₁₀ is the C₁-C₆₀ heteroaryl group), and the term “C₁-C₆₀ heteroalkylaryl group” as used herein refers to -A₁₁₁A₁₁₂ (A₁₁₁ is a C₁-C₅₅ heteroalkylene or heteroalkyl group, and A₁₁₂ is a C₁-C₅₉ heteroaryl group).

The term “monovalent non-aromatic condensed polycyclic group” as used herein refers to a monovalent group in which two or more rings are condensed with each other, only carbon is used as a ring-forming atom (for example, the number of carbon atoms may be 8 to 60) and the whole molecule is a non-aromaticity group. Examples of the monovalent non-aromatic condensed polycyclic group include a fluorenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group having two or more rings condensed to each other, a heteroatom N, O, P, Si, B, Se, or S, other than carbon atoms (for example, having 2 to 60 carbon atoms), as a ring-forming atom, and no aromaticity in its entire molecular structure. Examples of the monovalent non-aromatic condensed heteropolycyclic group include a carbazolyl group. The term “divalent non-aromatic heterocondensed polycyclic group” as used herein refers to a divalent group having the same structure as a monovalent non-aromatic heterocondensed polycyclic group.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ arylalkyl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, or the substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkylthio group, or a C₁-C₆₀ alkoxy group;

a C₁-C₆₀ alkyl group, a C₂-C₆₀ alkenyl group, a C₂-C₆₀ alkynyl group, a C₁-C₆₀ alkylthio group, or a C₁-C₆₀ alkoxy group, each independently substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or —P(═O)(Q₁₈)(Q₁₉);

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₇-C₆₀ alkylaryl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂₆)(Q₂₇), or —P(═O)(Q₂₈)(Q₂₉), or

—Si(Q₃₁)(Q₃₂)(Q₃₃), —N(Q₃₄)(Q₃₅), —B(Q₃₆)(Q₃₇), or —P(═O)(Q₃₈)(Q₃₉),

wherein Q₁ to Q₉, Q₁₁ to Q₁₉, Q₂₁ to Q₂₉, and Q₃₁ to Q₃₉ may each independently be hydrogen, a substituted or unsubstituted C₁-C₆₀ alkyl group, a substituted or unsubstituted C₂₋₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, or a substituted or unsubstituted monovalent non-aromatic condensed heteropolycyclic group.

The term “room temperature” used herein refers to a temperature of about 25° C.

The terms “a biphenyl group” and “a terphenyl group” as used herein refer to a monovalent group in which two or three benzene groups are bonded to each other through a single bond.

Hereinafter, a compound and an organic light-emitting device according to embodiments are described in detail with reference to Synthesis Example and Examples. However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1: Synthesis of Compound 1 (the Same as Compound 2-5-258 in the Table 5)

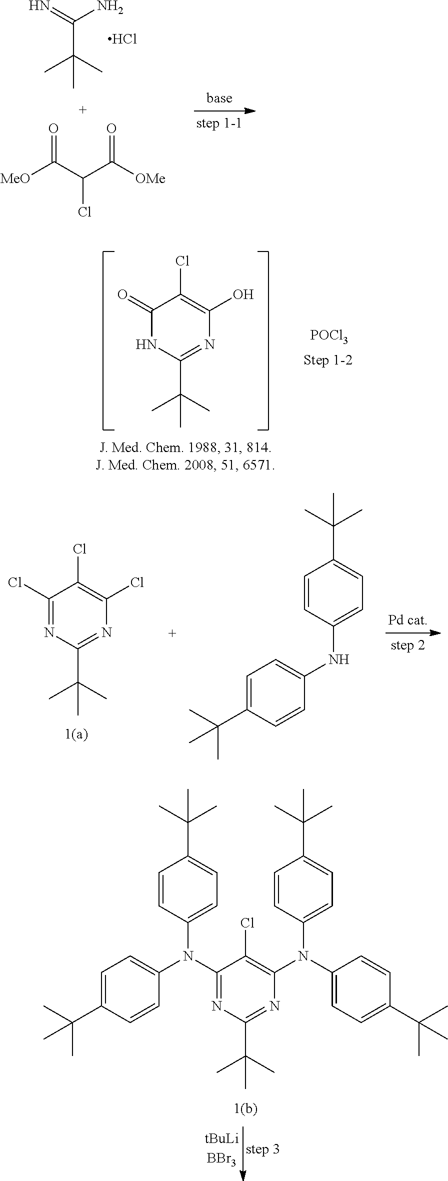

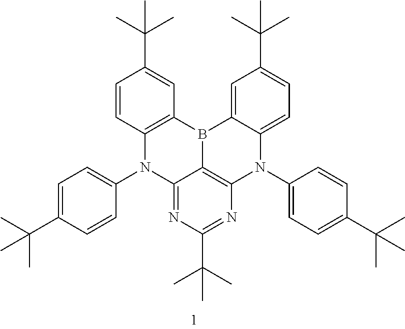 [SMILES: CC(C)(C)c1ccc(N2c3ccc(C(C)(C)C)cc3B3c4cc(C(C)(C)C)ccc4N(c4ccc(C(C)(C)C)cc4)c4nc(C(C)(C)C)nc2c43)cc1]

### Synthesis of Intermediate 1(a)

KOtBu (potassium tert-butoxide, 9.01 grams (g), 128 millimole (mmol)) was loaded into a reaction vessel and charged with nitrogen. 80 milliliter (mL) of methanol and tert-butylcarbamidine hydrochloride (5.76 g, 42 mmol) were added thereto and then dissolved at room temperature. 15 minutes after, dimethyl chloromalonate (6.8 mL, 42 mmol) was additionally added to the reaction vessel to cause a reaction, followed by stirring for 4 hours at a temperature of 65° C. After the reaction was completed, the resultant mixture was cooled to room temperature, followed by primary extraction using diethyl ether and water, and secondary extraction using methylchoride (MC) and HBr. Finally, the organic layer obtained after removing the remaining salt therefrom by using an aqueous sodium hydroxide solution was dried using anhydrous magnesium sulfate (MgSO₄) and concentrated. Without further purification, the resulting yellow solid was mixed with POCl₃ (20 mL) and reacted at a temperature of 120° C. for 15 hours. After the reaction was completed, the resultant mixture was washed with an aqueous sodium hydroxide solution and an aqueous sodium hydrogen carbonate solution. An extraction process was performed thereon by using MC and water, and the obtained organic layer was filtered through a filter and concentrated. The solid compound obtained therefrom was directly used in the next reaction without further purification.

LC-Mass (calculated: 237.98 g/mol, found: 238.9 g/mol (M+1)) Synthesis of Intermediate 1(b)

Intermediate 1(a) (2.0 g, 8.2 mmol), bis(4-tert-butyl)phenyl)amine (5.2 g, 18.4 mmol), Pd₂(dba)₃ (Tris(dibenzylideneacetone)dipalladium(0), 0.38 g, 0.41 mmol), SPhos (([2-dicyclohexylphosphino-2′,6′-dimethoxybiphenyl, 0.36 g, 0.85 mmol), and NaOtBu (sodium tert-butoxide, 1.95 g, 20.6 mmol) were added to a reaction vessel and dissolved in 80 mL of toluene, and stirred at a temperature of 110° C. After completion of the reaction, the reaction solution was cooled to room temperature, and then the obtained organic layer was filtered and concentrated using a Celite pad. The solid compound obtained therefrom was separated by column chromatography (hexane/ethylacetate 12%). The resulting pale yellow solid was obtained with a yield of 1.44 g (24%).

LC-Mass (calculated: 728.46 g/mol, found: 729.4 g/mol (M+1))

### Synthesis of Compound 1

Intermediate 1(b) (0.47 g, 0.64 mmol) was loaded in a reaction vessel which was able to be vacuumed, and was charged three times with vacuum and nitrogen. While the temperature of the reaction vessel was maintained at a temperature of −78° C. by using dry ice, tert-Butyl benzene (9.5 mL) was added thereto. tert-butyl lithium (1.47 molar (M) in pentane, 1.2 mL, 1.76 mmol) was added to the reaction vessel and reacted at a temperature of −60° C. for 1 hour. The temperature was adjusted to be −78° C., and then, 0.20 mL (2.07 mmol) of BBr₃ was added thereto. The temperature was gradually increased to room temperature, and then, N,N-diisopropylethylamine (0.40 mL, 2.29 mmol) was added thereto. The mixture of the reaction vessel was reacted at a temperature of 110° C. for 15 hours. After completion of the reaction, the reaction solution was cooled to room temperature, and then the obtained organic layer was filtered and concentrated using a Florisil® pad. The solid compound obtained therefrom was separated by column chromatography (hexane/MC). The resulting pale yellow solid was obtained with a yield of 0.04 g (10%).

LC-Mass (calculated: 702.48 g/mol, found: 703.4 g/mol (M+1))

### Synthesis Example 2: Synthesis of Compound 3 (the Same as Compound 2-25-042 in the Table 25)

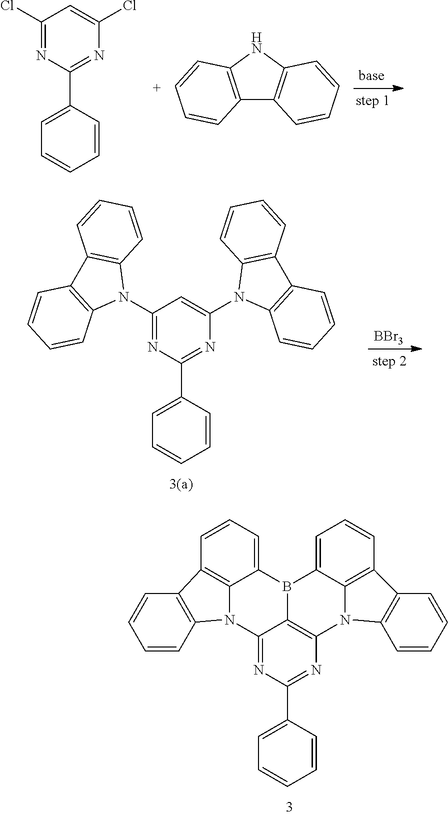

### Synthesis of Intermediate 3(a)

Carbazole (4.36 g, 26 mmol) was loaded in a reaction vessel which was able to be vacuumed, and was charged three times with vacuum and nitrogen. NaH (60% in oil, 1.03 g, 26 mmol) was dissolved in 30 mL of DMF (dimethylformamide) and then loaded in the reaction vessel. After stirring at room temperature for 30 minutes, fenclorim (1.92 g, 8.6 mmol) was added thereto and then reacted at a temperature of 120° C. for 3 hours. After completion of the reaction, a precipitation process was performed thereon using water and then, an extraction process was performed thereon using methylchloride (MC), and the solvent of the obtained organic layer was dried with anhydrous magnesium sulfate (MgSO₄) and concentrated. The solid compound obtained therefrom was separated by column chromatography (hexane/EA, 12%) to obtain Intermediate 3(a) with a yield of 0.45 g (11%).

LC-Mass (calculated: 486.18 g/mol, found: 487.2 g/mol (M+1)) Synthesis of Compound 3

Intermediate 3(a) (0.17 g, 0.35 mmol) was loaded in a reaction vessel which was able to be vacuumed, and was charged three times with vacuum and nitrogen. 4 mL of o-dichlorobenzene and 0.08 mL of BBr₃ were loaded into the reaction vessel at room temperature, and reacted at a temperature of 140° C. for 15 hours. After completion of the reaction, diisopropylethylamine (0.40 mL, 2.29 mmol) was added at a temperature of 0° C. using ice. After two hours of stirring at room temperature, an organic layer was separated therefrom using an organic solvent, and then the solvent of the obtained organic layer was dried using anhydrous magnesium sulfate (MgSO₄) and concentrated. The solid compound obtained therefrom was separated by column chromatography (hexane/MC).

As a result, pale yellow solid was obtained.

LC-Mass (calculated: 494.17 g/mol, found: 495.2 g/mol (M+1))

### Evaluation Example 1: Material Property Evaluation

Measurements were made according to the method as below. Results thereof are shown in Table 26.
Optical band gap Eg evaluation method: Each compound was diluted in toluene to a concentration of 1×10⁻⁵ M, and the ultraviolet (UV) absorption spectrum thereof was measured at room temperature by using a Shimadzu UV-350 spectrometer. Then, the lowest unoccupied molecular orbital (LUMO) energy level thereof was calculated by using the optical band gap (Eg) from the edge of the absorption spectrum and HOMO energy level. ΔEg was analyzed by the calculation of the gap of HOMO energy level and LUMO energy level.
S₁ energy level evaluation method: Regarding a mixture of toluene and each compound (diluted to a concentration of 1×10⁻⁴M), a photoluminescence spectrum thereof was measured at room temperature using a photoluminescence spectrometer, and the identified peak was analyzed to calculate the on set S1 energy level. Maximum peak was analyzed to calculate the S1,max energy.
Photoluminescence (PL) spectrum measurement method: Each compound was diluted to a concentration of 10⁻⁴ M in toluene, and a PL spectrum thereof was measured (@ 298K) using a F7000 spectrofluorometer equipped with a Xenon lamp, obtained from Hitachi Inc. From the results, the full width at half maximum (FWHM) of each compound was measured.

TABLE 26

| Compound | | S_{1,max} | S1 onset | PL | FWHM |
|---|---|---|---|---|---|
| No. | ΔE_{g} | (eV) | (eV) | (nm) | (nm) |
| Compound A | 2.71 | 2.737 | 2.87 | 453 | 22 |
| 1 | 3.171 | 3.188 | 3.289 | 389 | 18 |

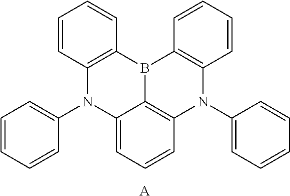

From Table 26, it can be confirmed that the heterocyclic compound represented by Formula 1 has a material skeleton that is advantageous for blue due to the shift to a shorter wavelength caused by pyrimidine.

### Evaluation Example 2: Calculation of the Material Property Evaluation

HOMO energy level, LUMO energy level, energy gap, T1 energy level, S1 energy level, gap between S1 and T1, and Oscillator strength of some of the heterocyclic compounds represented by Formula 1-1 or 1-2 was evaluated using the Gaussian 09 program accompanying molecular structure optimization by density functional theory (DFT) based on B3LYP, and the results are shown in Table 27 below.

TABLE 27

| | | | | | | Oscillator |
|---|---|---|---|---|---|---|
| Compound | | | | | | strength |
| No. | HOMO | LUMO | S1 | T1 | ΔE_{ST} | (f) |
| A | −4.754 | −1.092 | 3.071 | 2.614 | 0.457 | 0.309 |
| B | −6.471 | −2.500 | 3.126 | 2.848 | 0.277 | 0.015 |
| C | −6.987 | −2.876 | 3.524 | 2.782 | 0.742 | 0.357 |
| D | −6.117 | −2.332 | 2.891 | 2.592 | 0.299 | 0.020 |
| E | −6.136 | −2.304 | 3.241 | 2.760 | 0.481 | 0.075 |
| 1 | −5.33 | −1.425 | 3.295 | 2.875 | 0.420 | 0.390 |
| 3 | −5.802 | −2.097 | 3.208 | 2.747 | 0.461 | 0.573 |

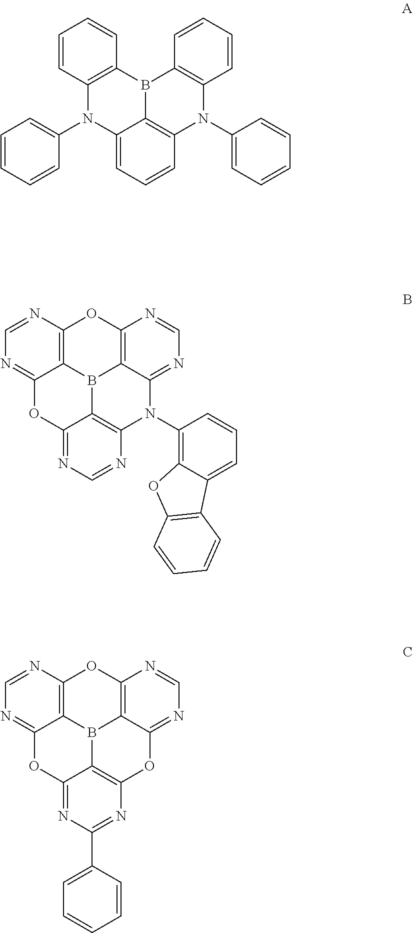 [SMILES: c1ccc(N2c3ccccc3B3c4ccccc4N(c4ccccc4)c4cccc2c43)cc1 ; c1ccc2c(c1)oc1c(N3c4ncnc5c4B4c6c(ncnc6Oc6ncnc3c64)O5)cccc12 ; c1ccc(-c2nc3c4c(n2)Oc2ncnc5c2B4c2c(ncnc2O3)O5)cc1]

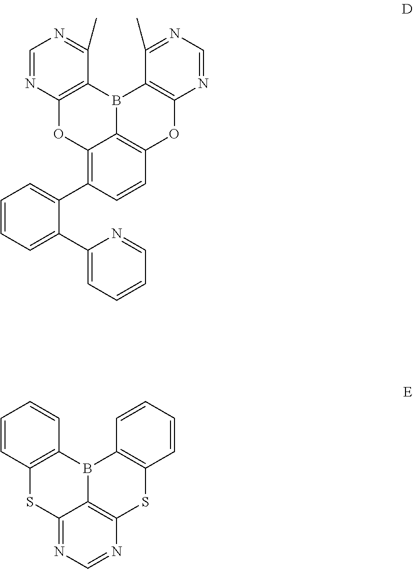 [SMILES: Cc1ncnc2c1B1c3c(C)ncnc3Oc3c(-c4ccccc4-c4ccccn4)ccc(c31)O2 ; c1ccc2c(c1)Sc1ncnc3c1B2c1ccccc1S3]

In the case of Compound A, the HOMO level is too high to cause a problem with the electron transport, and in Compound B, D, and E, the oscillator strength is low and thus the luminous efficiency is low. In the case of Compound D, the triplet energy is particularly low, and the efficiency characteristics are considered to be very low. In the case of Compound C, DEST is 0.7 eV or more and thus it is not able to utilize the triplet energy by the reverse intersystem crossing and the internal quantum efficiency will be less than about 25%, and due to the structure it is expected to hardly express high efficiency in deep blue. In the calculation results of Compounds 1 and 3 performed to compensate for the disadvantages of the above compound A to E, the characteristics of luminous efficiency are increased by increasing the oscillator strength (f) value, and the triplet energy is maximized by maintaining the gap between the triplet energy and the singlet energy for the reverse intersystem crossing. By designing to have a specific material structure, it can be expected to improve the stability of the material while having high-efficiency emission characteristics in blue

An organic light-emitting device containing the heterocyclic compound can have high or improved efficiency, long or improved lifespan, low driving voltage and high or improved color purity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

An organic light-emitting device containing the heterocyclic compound can have high or improved efficiency, long or improved lifespan, low driving voltage and high or improved color purity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present detailed description as defined by the following claims.

What is claimed is:

1. A heterocyclic compound represented by Formula 1-1 or 1-2: 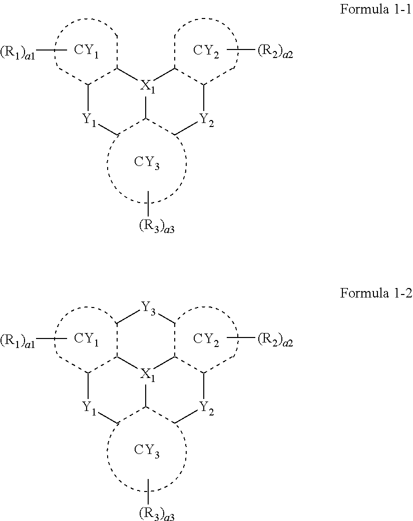
wherein, in Formulae 1-1 and 1-2
X₁ is B or N,
ring CY₁, ring CY₂, and ring CY₃ are each independently a C₅-C₃₀ carbocyclic group or a C₁-C₃₀ heterocyclic group,
at least one of ring CY₁, ring CY₂, and ring CY₃ is a pyrimidine group,
Y₁, Y₂, and Y₃ are each independently O, S, Se, B(R₄), N(R₄), C(R₄)(R₅), Si(R₄)(R₅), Ge(R₄)(R₅), P(R₄), P(═O)(R₄), P(═S)(R₄), or Al(R₄),
R₁, R₂, R₃, R₄, and R₅ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
two neighboring groups from among R₁ to R₅ are optionally bonded to each other to form a C₅-C₃₀ carbocyclic group unsubstituted or substituted with R₁₀ or a C₁-C₃₀ heterocyclic group unsubstituted or substituted with R₁₀, and R₁₀ is the same as described in connection with R₁,
a1 to a3 are each independently an integer from 1 to 10,
any one of the following condition (i) to (iii) is satisfied:
(i) when any one of ring CY₁, ring CY₂, and ring CY₃ contains pyrimidine, at least one of Y₁ to Y₃ is N(R₄);
(ii) when two of ring CY₁, ring CY₂, and ring CY₃ each contain pyrimidine, at least one of Y₁ to Y₃ is N(R₄); and
(iii) when each of ring CY₁, ring CY₂, and ring CY₃ contains pyrimidine, Y₁ to Y₃ are each independently Se, B(R₄), N(R₄), C(R₄)(R₅), Si(R₄)(R₅), Ge(R₄)(R₅), P(R₄), P(═O)(R₄), P(═S)(R₄), or Al(R₄),
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ arylalkyl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkylthio group, or a C₁-C₆₀ alkoxy group;
a C₁-C₆₀ alkyl group, a C₂-C₆₀ alkenyl group, a C₂-C₆₀ alkynyl group, a C₁-C₆₀ alkylthio group, or a C₁-C₆₀ alkoxy group, each independently substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or —P(═O)(Q₁₈)(Q₁₉);
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, a C₆-C₆₀ aryloxy group, a C₆-C₆₀ arylthio group, a C₇-C₆₀ alkylaryl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, or a monovalent non-aromatic condensed heteropolycyclic group, each independently substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ arylalkyl group, C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅)-B(Q₂₆)(Q₂₇), or —P(═O)(Q₂₈)(Q₂₉); or
—Si(Q₃₁)(Q₃₂)(Q₃₃), —N(Q₃₄)(Q₃₅), —B(Q₃₆)(Q₃₇), or —P(═O)(Q₃₈)(Q₃₉),
wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, or a substituted or unsubstituted monovalent non-aromatic condensed heteropolycyclic group.

2. The heterocyclic compound of claim 1, wherein
X₁ is B.

3. The heterocyclic compound of claim 1, wherein
ring CY₁, ring CY₂, and ring CY₃ are each independently group A, group B, a condensed ring in which two or more groups of group A are condensed with each other, a condensed ring in which two or more groups of group B are condensed with each other, or a condensed ring in which at least one group A is condensed with at least one group B, wherein
group A is a cyclopenta-1,3-diene group, an indene group, an azulene group, a benzene group, a naphthalene group, an anthracene group, a phenanthrene group, a tetracene group, a tetraphene group, a pyrene group, a chrysene group, a triphenylene group, or a fluorene group, and
group B is a furan group, a thiophene group, a pyrrole group, borole group, a silole group, a pyrrolidine group, an imidazole group, a triazole group, an oxazole group, an isoxazole group, a thiazole group, an isothiazole group, a pyridine group, a pyrimidine group, a pyridazine group, a triazine group, an indole group, an isoindole group, an indolizine group, a quinoline group, an isoquinoline group, a quinoxaline group, an isoquinoxaline group, a carbazole group, a dibenzofuran group, a dibenzothiophene group, a dibenzosilole group, or a dibenzoborole group.

4. The heterocyclic compound of claim 1, wherein
ring CY₁, ring CY₂, and ring CY₃ are each independently a benzene group, a naphthalene group, a fluorene group, a pyridine group, a pyrimidine group, a pyrazine group, a quinoline group, an isoquinoline group, a quinazoline group, a quinoxaline group, a phthalazine group, a carbazole group, an azacarbazole group, a dibenzofuran group, a dibenzothiophene group, a dibenzoselenophene group, a dibenzosilole group, an azadibenzofuran group, an azadibenzothiophene group, an azadibenzoselenophene group, or an azadibenzosilole group.

5. The heterocyclic compound of claim 1, wherein
at least one of Y₁ to Y₃ is N(R₄).

6. The heterocyclic compound of claim 1, wherein
X₁ is B, and
Y₁ and Y₂ are each N(R₄).

7. The heterocyclic compound of claim 1, wherein
R₁ to R₅ are each independently:
hydrogen, deuterium, —F, —Cl, —Br, —I, a hydroxyl group, a cyano group (CN), a nitro group, an amino group, a C₁-C₆₀ alkyl group, a C₁-C₆₀ alkylthio group, or a C₁-C₆₀ alkoxy group;
a C₁-C₆₀ alkyl group, a C₁-C₆₀ alkylthio group, or a C₁-C₆₀ alkoxy group, each independently substituted with at least one of deuterium, —F, —Cl, —Br, —I, a hydroxyl group, a cyano group (CN), a nitro group, an amino group, a phenyl group, a biphenyl group, a naphthyl group, a fluorenyl group, a phenanthrenyl group, an anthracenyl group, a fluoranthenyl group, a triphenylenyl group, a pyrenyl group, or a chrysenyl group;
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group;
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each independently substituted with at least one of deuterium, —F, —Cl, —Br, —I, a hydroxyl group, a cyano group, a nitro group, an amino group, a C₁-C₆₀ alkyl group, a C₁-C₆ alkoxy group, a C₁-C₆₀ alkylthio group, a C₇-C₆₀ arylalkyl group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a carbazolyl group, —Si(Q₃₁)(Q₃₂)(Q₃₃), —N(Q₃₄)(Q₃₅), —B(Q₃₆)(Q₃₇), or —P(═O)(Q₃₈)(Q₃₉); or
—N(Q₁)(Q₂), —Si(Q₃)(Q₄)(Q₅), —B(Q₆)(Q₇), or —P(═O)(Q₈)(Q₉).

8. The heterocyclic compound of claim 1, wherein
R₄ and R₅ are each independently:
hydrogen, deuterium, —F, —Cl, —Br, —I, a hydroxyl group, a cyano group (CN), a nitro group, an amino group, a C₁-C₆₀ alkyl group, a C₁-C₆₀ alkylthio group, or a C₁-C₆₀ alkoxy group;
a C₁-C₆₀ alkyl group, a C₁-C₆₀ alkylthio group, or a C₁-C₆₀ alkoxy group, each independently substituted with at least one of deuterium, —F, —Cl, —Br, —I, a hydroxyl group, a cyano group (CN), a nitro group, an amino group, or a phenyl group; or
a group represented by one of Formulae 4-1 to 4-42: 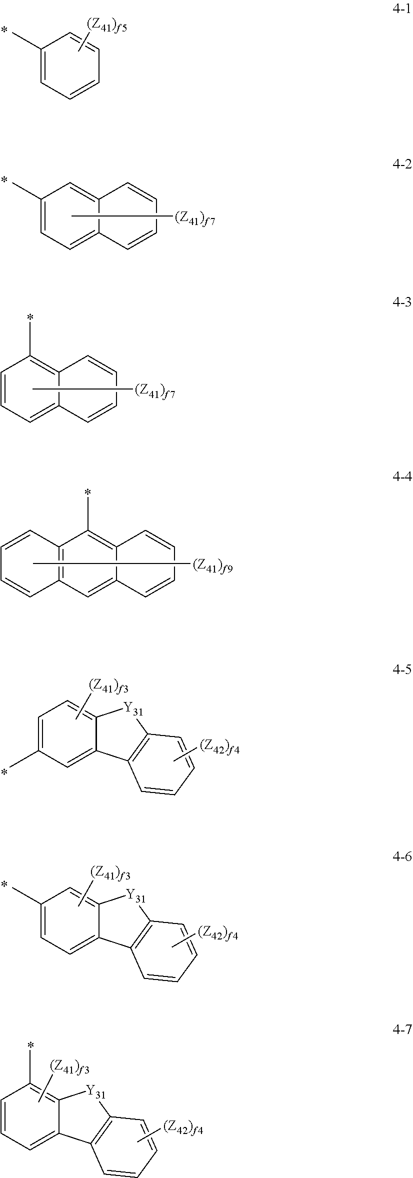 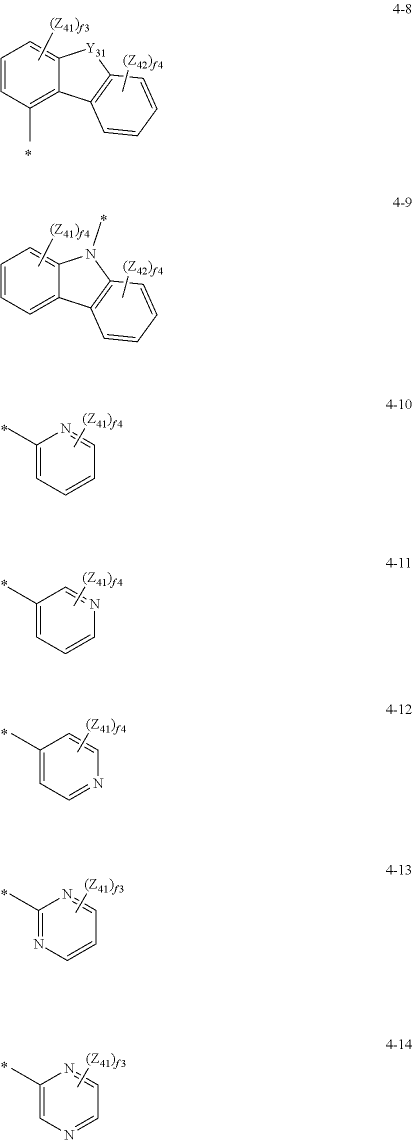 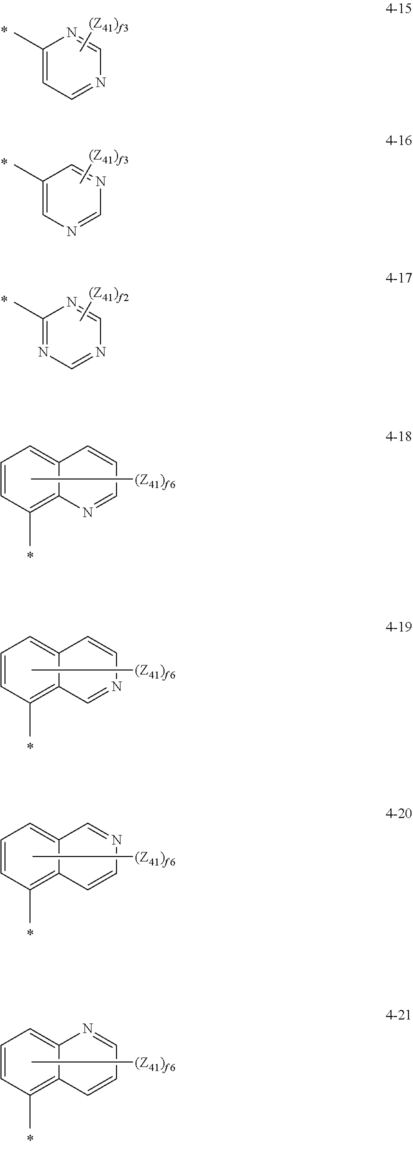 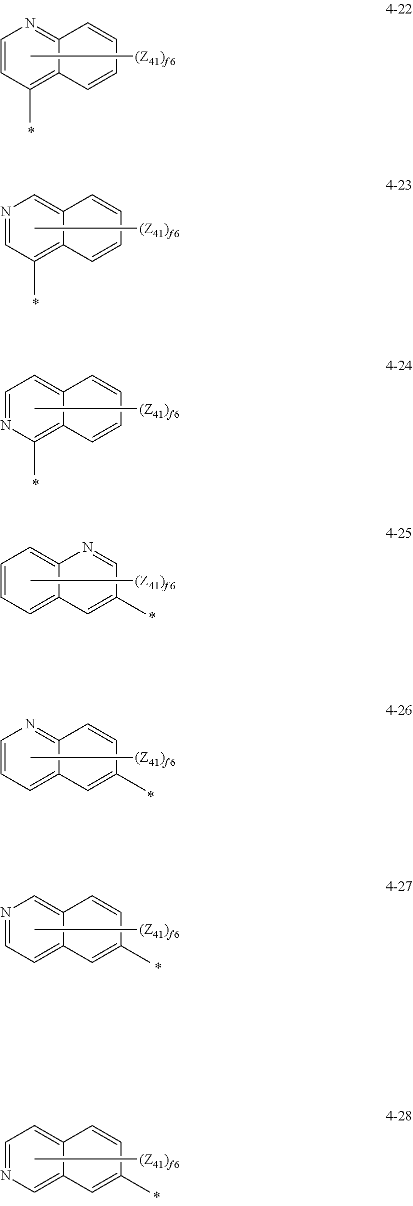 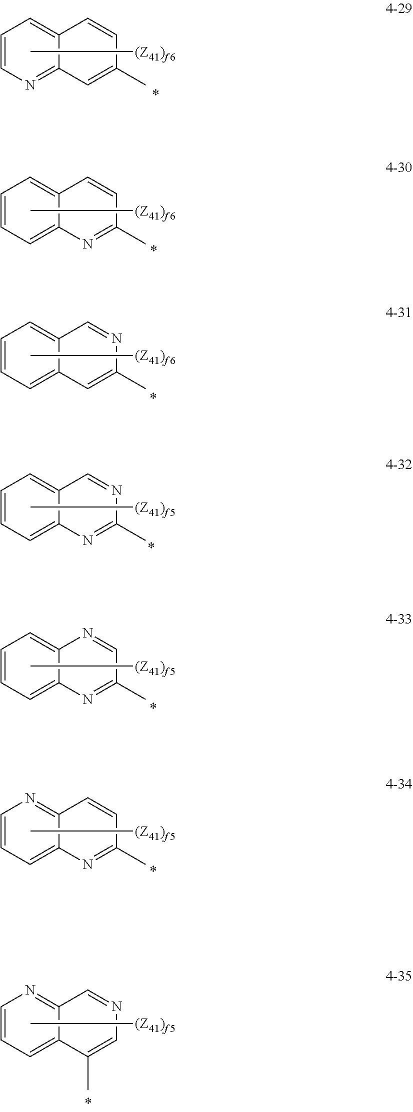 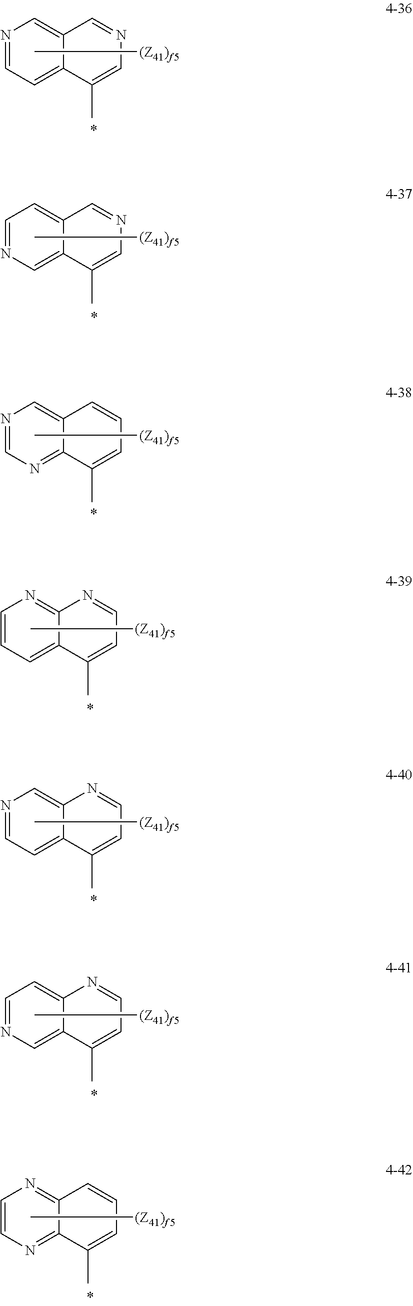
wherein, in Formulae 4-1 to 4-42,
Y₃₁ is O, S, C(Z₄₃)(Z₄₄), N(Z₄₃), or Si(Z₄₃)(Z₄₄),
Z₄₁ to Z₄₄ are each independently:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a C₇-C₆₀ arylalkyl group, a cyclopentyl group, a cyclohexyl group, a cycloheptyl group, a cyclopentenyl group, a cyclohexenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group;
a cyclopentyl group, a cyclohexyl group, a cycloheptyl group, a cyclopentenyl group, a cyclohexenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each independently substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, or a C₇-C₆₀ arylalkyl group,
f3 is an integer from 1 to 3,
f4 is an integer from 1 to 4,
f5 is an integer from 1 to 5,
f6 is an integer from 1 to 6,
f7 is an integer from 1 to 7,
f9 is an integer from 1 to 9,
* indicates a binding site to a neighboring atom, and
substituent Z₄₁ is present on each ring through which the bond with the substituent Z₄₁ passes.

9. The heterocyclic compound of claim 8, wherein
R₄ and R₅ are each independently:
hydrogen, deuterium, —F, —Cl, —Br, —I, a hydroxyl group, a cyano group, a nitro group, a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group; or
a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group, each independently substituted with at least one of deuterium, —F, —Cl, —Br, —I, a hydroxyl group, a cyano group, a nitro group, or a phenyl group; or
a group represented by one of Formulae 4-1 to 4-8 of claim 8, wherein
Y₃₁ in Formulae 4-5 to 4-8 is S, C(Z₄₃)(Z₄₄), N(Z₄₃), or Si(Z₄₃)(Z₄₄).

10. The heterocyclic compound of claim 1, wherein
R₁ to R₃ are each independently:
hydrogen, deuterium, —F, —Cl, —Br, —I, a hydroxyl group, a cyano group, a nitro group, a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group; or
a methyl group, an ethyl group, an n-propyl group, an iso-propyl group, an n-butyl group, an iso-butyl group, a sec-butyl group, a tert-butyl group, an n-pentyl group, a tert-pentyl group, a neo-pentyl group, an iso-pentyl group, a sec-pentyl group, a 3-pentyl group, a sec-isopentyl group, an n-hexyl group, an iso-hexyl group, a sec-hexyl group, or a tert-hexyl group, each independently substituted with at least one of deuterium, —F, —Cl, —Br, —I, a hydroxyl group, a cyano group, a nitro group, or a phenyl group.

11. The heterocyclic compound of claim 1, wherein
ring CY₃ is a pyrimidine group, and each of ring CY₁ and ring CY₂ is a benzene group;
each of ring CY₁ and ring CY₂ is a pyrimidine group, and ring CY₃ is a benzene group; or
each of ring CY₁, ring CY₂, and ring CY₃ is a pyrimidine group.

12. The heterocyclic compound of claim 1, wherein
the heterocyclic compound is represented by any one of Formulae 2-1 to 2-25: 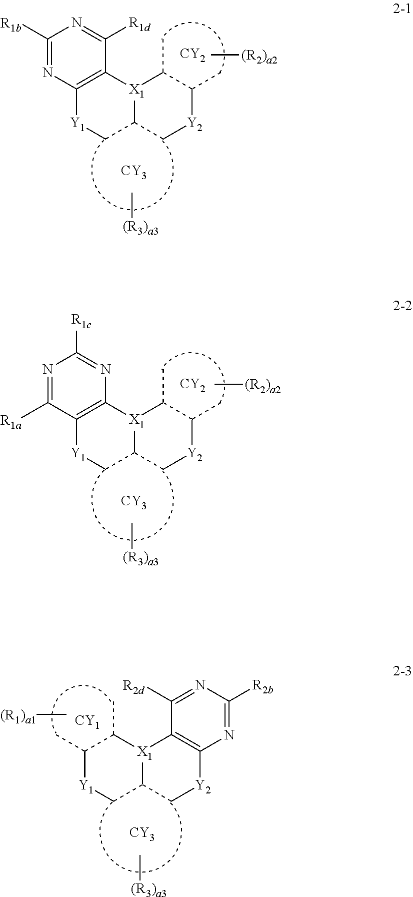 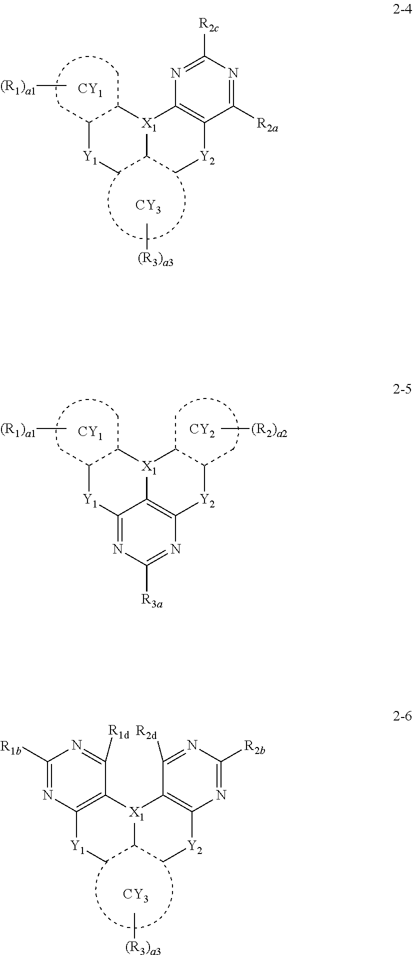 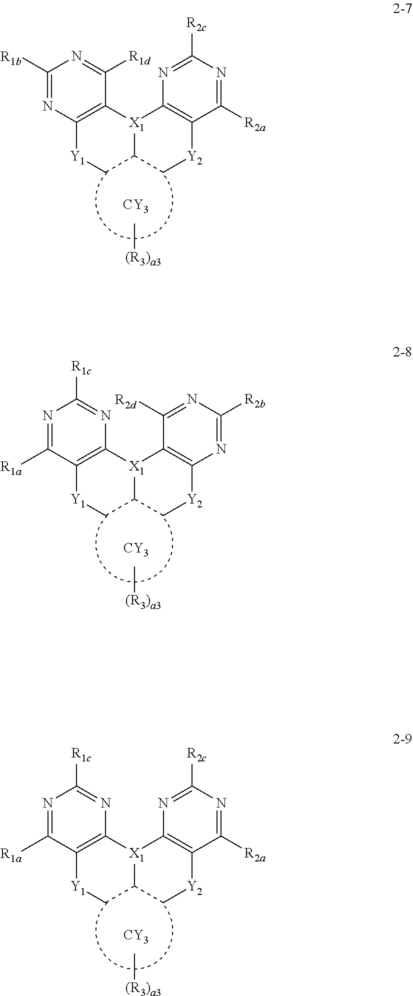 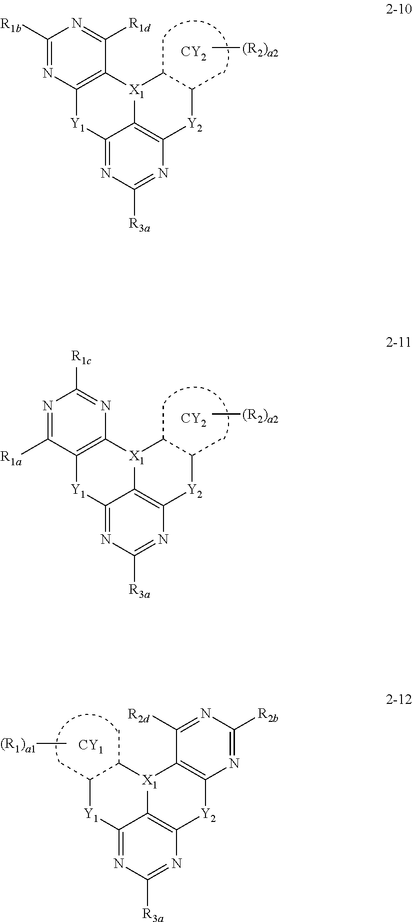 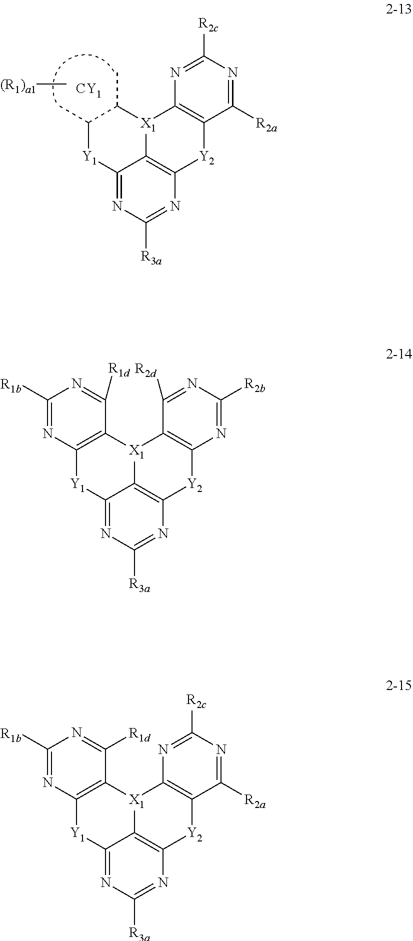 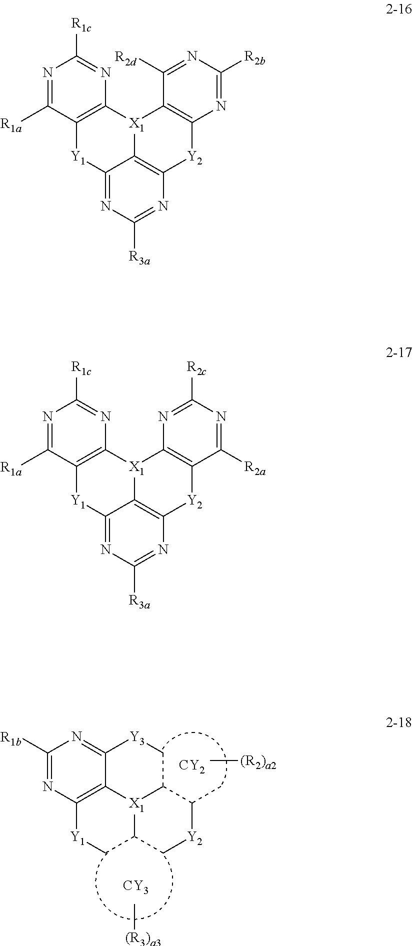 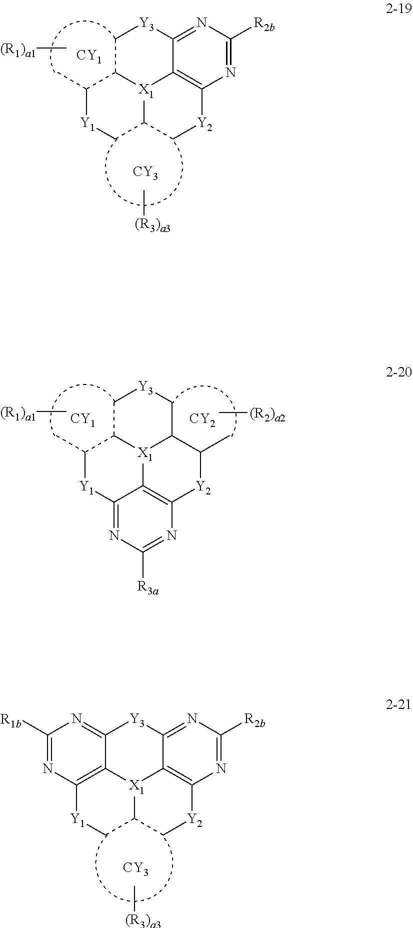 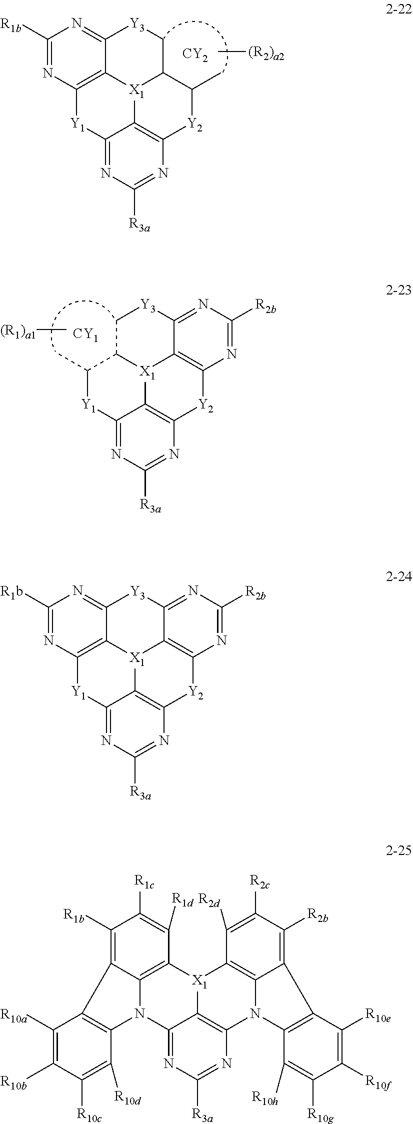
wherein, in Formulae 2-1 to 2-25,
ring CY₁, ring CY₂, ring CY₃, X₁, Y₁, Y₂, Y₃, R₁, R₂, R₃, a1, a2, and a3 are the same as described in claim 1,
R₁ₐ to R_{1d} are each independently the same as described in connection with R₁ in claim 1,
R₂ₐ to R_{2d} are each independently the same as described in connection with R₂ in claim 1,
R₃ₐ is the same as described in connection with R₃ in claim 1, and
R₁₀ₐ to R₁₀ₕ are each independently the same as described in connection with R₁₀ in claim 1.

13. The heterocyclic compound of claim 12, wherein
in Formulae 2-1 to 2-13 and 2-18 to 2-23,
ring CY₁ to ring CY₃ are each independently a benzene group, a naphthalene group, or a fluorene group.

14. The heterocyclic compound of claim 1, wherein
the heterocyclic compound is the compounds of Groups 2-1 to 2-25:
Group 2-1
The compounds are represented by the formula 2-1 below, wherein CY₂ and CY₃ are each a benzene group, X₁ is B, and Y1, Y2, R1b, R1d, (R2)a2, (R3)a3 are each shown in the table 1 below, 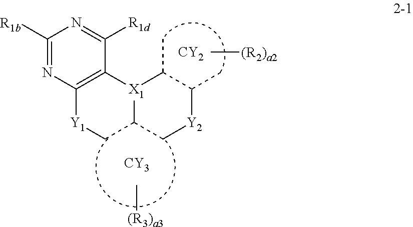
TABLE 1

| Compound No. | Y1 | Y2 | R1b | R1d | (R2)a2 | (R3)a3 |
|---|---|---|---|---|---|---|
| 2-1-001 | N—Ph | N—Ph | H | H | H | H |
| 2-1-002 | N—Ph | N—Ph | t-bu | H | H | H |
| 2-1-003 | N—Ph | N—Ph | methyl | H | H | H |
| 2-1-004 | N—Ph | N—Ph | t-bu | t-bu | t-bu | t-bu |
| 2-1-005 | N—Ph | N—Ph | methyl | methyl | methyl | methyl |
| 2-1-006 | N—Ph | N—Ph | b1 | H | H | H |
| 2-1-007 | N—Ph | N—Ph | b2 | H | H | H |
| 2-1-008 | N—Ph | N—Ph | b3 | H | H | H |
| 2-1-009 | N—Ph | N—Ph | H | H | H | a1 |
| 2-1-010 | N—Ph | N—Ph | t-bu | H | H | a1 |
| 2-1-011 | N—Ph | N—Ph | methyl | H | H | a1 |
| 2-1-012 | N—Ph | N—Ph | b1 | H | H | a1 |
| 2-1-013 | N—Ph | N—Ph | b2 | H | H | a1 |
| 2-1-014 | N—Ph | N—Ph | b3 | H | H | a1 |
| 2-1-015 | N—Ph | N—Ph | H | H | H | a2 |
| 2-1-016 | N—Ph | N—Ph | t-bu | H | H | a2 |
| 2-1-017 | N—Ph | N—Ph | methyl | H | H | a2 |
| 2-1-018 | N—Ph | N—Ph | b1 | H | H | a2 |
| 2-1-019 | N—Ph | N—Ph | b2 | H | H | a2 |
| 2-1-020 | N—Ph | N—Ph | b3 | H | H | a2 |
| 2-1-021 | N—Ph | N—Ph | H | H | H | a3 |
| 2-1-022 | N—Ph | N—Ph | t-bu | H | H | a3 |
| 2-1-023 | N—Ph | N—Ph | methyl | H | H | a3 |
| 2-1-024 | N—Ph | N—Ph | b1 | H | H | a3 |
| 2-1-025 | N—Ph | N—Ph | b2 | H | H | a3 |
| 2-1-026 | N—Ph | N—Ph | b3 | H | H | a3 |
| 2-1-027 | N—Ph | N—Ph | H | H | H | a4 |
| 2-1-028 | N—Ph | N—Ph | t-bu | H | H | a4 |
| 2-1-029 | N—Ph | N—Ph | methyl | H | H | a4 |
| 2-1-030 | N—Ph | N—Ph | b1 | H | H | a4 |
| 2-1-031 | N—Ph | N—Ph | b2 | H | H | a4 |
| 2-1-032 | N—Ph | N—Ph | b3 | H | H | a4 |
| 2-1-033 | N—Ph | N—Ph | H | H | H | a5 |
| 2-1-034 | N—Ph | N—Ph | t-bu | H | H | a5 |
| 2-1-035 | N—Ph | N—Ph | methyl | H | H | a5 |
| 2-1-036 | N—Ph | N—Ph | b1 | H | H | a5 |
| 2-1-037 | N—Ph | N—Ph | b2 | H | H | a5 |
| 2-1-038 | N—Ph | N—Ph | b3 | H | H | a5 |
| 2-1-039 | N—Ph | N—Ph | H | H | H | a6 |
| 2-1-040 | N—Ph | N—Ph | t-bu | H | H | a6 |
| 2-1-041 | N—Ph | N—Ph | methyl | H | H | a6 |
| 2-1-042 | N—Ph | N—Ph | b1 | H | H | a6 |
| 2-1-043 | N—Ph | N—Ph | b2 | H | H | a6 |
| 2-1-044 | N—Ph | N—Ph | b3 | H | H | a6 |
| 2-1-045 | N—Ph | N—Ph | H | H | a1 | H |
| 2-1-046 | N—Ph | N—Ph | t-bu | H | a1 | H |
| 2-1-047 | N—Ph | N—Ph | methyl | H | a1 | H |
| 2-1-048 | N—Ph | N—Ph | b1 | H | a1 | H |
| 2-1-049 | N—Ph | N—Ph | b2 | H | a1 | H |
| 2-1-050 | N—Ph | N—Ph | b3 | H | a1 | H |
| 2-1-051 | N—Ph | N—Ph | H | H | a2 | H |
| 2-1-052 | N—Ph | N—Ph | t-bu | H | a2 | H |
| 2-1-053 | N—Ph | N—Ph | methyl | H | a2 | H |
| 2-1-054 | N—Ph | N—Ph | b1 | H | a2 | H |
| 2-1-055 | N—Ph | N—Ph | b2 | H | a2 | H |
| 2-1-056 | N—Ph | N—Ph | b3 | H | a2 | H |
| 2-1-057 | N—Ph | N—Ph | H | H | a3 | H |
| 2-1-058 | N—Ph | N—Ph | t-bu | H | a3 | H |
| 2-1-059 | N—Ph | N—Ph | methyl | H | a3 | H |
| 2-1-060 | N—Ph | N—Ph | b1 | H | a3 | H |
| 2-1-061 | N—Ph | N—Ph | b2 | H | a3 | H |
| 2-1-062 | N—Ph | N—Ph | b3 | H | a3 | H |
| 2-1-063 | N—Ph | N—Ph | H | H | a4 | H |
| 2-1-064 | N—Ph | N—Ph | t-bu | H | a4 | H |
| 2-1-065 | N—Ph | N—Ph | methyl | H | a4 | H |
| 2-1-066 | N—Ph | N—Ph | b1 | H | a4 | H |
| 2-1-067 | N—Ph | N—Ph | b2 | H | a4 | H |
| 2-1-068 | N—Ph | N—Ph | b3 | H | a4 | H |
| 2-1-069 | N—Ph | N—Ph | H | H | a5 | H |
| 2-1-070 | N—Ph | N—Ph | t-bu | H | a5 | H |
| 2-1-071 | N—Ph | N—Ph | methyl | H | a5 | H |
| 2-1-072 | N—Ph | N—Ph | b1 | H | a5 | H |
| 2-1-073 | N—Ph | N—Ph | b2 | H | a5 | H |
| 2-1-074 | N—Ph | N—Ph | b3 | H | a5 | H |
| 2-1-075 | N—Ph | N—Ph | H | H | a6 | H |
| 2-1-076 | N—Ph | N—Ph | t-bu | H | a6 | H |
| 2-1-077 | N—Ph | N—Ph | methyl | H | a6 | H |
| 2-1-078 | N—Ph | N—Ph | b1 | H | a6 | H |
| 2-1-079 | N—Ph | N—Ph | b2 | H | a6 | H |
| 2-1-080 | N—Ph | N—Ph | b3 | H | a6 | H |
| 2-1-081 | N—Ph | N—Ph | H | H | b1 | H |
| 2-1-082 | N—Ph | N—Ph | t-bu | H | b1 | H |
| 2-1-083 | N—Ph | N—Ph | methyl | H | b1 | H |
| 2-1-084 | N—Ph | N—Ph | b1 | H | b1 | H |
| 2-1-085 | N—Ph | N—Ph | b2 | H | b1 | H |
| 2-1-086 | N—Ph | N—Ph | b3 | H | b1 | H |
| 2-1-087 | N—Ph | N—Ph | H | H | b2 | H |
| 2-1-088 | N—Ph | N—Ph | t-bu | H | b2 | H |
| 2-1-089 | N—Ph | N—Ph | methyl | H | b2 | H |
| 2-1-090 | N—Ph | N—Ph | b1 | H | b2 | H |
| 2-1-091 | N—Ph | N—Ph | b2 | H | b2 | H |
| 2-1-092 | N—Ph | N—Ph | b3 | H | b2 | H |
| 2-1-093 | N—Ph | N—Ph | H | H | b3 | H |
| 2-1-094 | N—Ph | N—Ph | t-bu | H | b3 | H |
| 2-1-095 | N—Ph | N—Ph | methyl | H | b3 | H |
| 2-1-096 | N—Ph | N—Ph | b1 | H | b3 | H |
| 2-1-097 | N—Ph | N—Ph | b2 | H | b3 | H |
| 2-1-098 | N—Ph | N—Ph | b3 | H | b3 | H |
| 2-1-099 | N—Ph | N—Ph | H | H | b4 | H |
| 2-1-100 | N—Ph | N—Ph | t-bu | H | b4 | H |
| 2-1-101 | N—Ph | N—Ph | methyl | H | b4 | H |
| 2-1-102 | N—Ph | N—Ph | b1 | H | b4 | H |
| 2-1-103 | N—Ph | N—Ph | b2 | H | b4 | H |
| 2-1-104 | N—Ph | N—Ph | b3 | H | b4 | H |
| 2-1-105 | N—Ph | N—Ph | H | H | b5 | H |
| 2-1-106 | N—Ph | N—Ph | t-bu | H | b5 | H |
| 2-1-107 | N—Ph | N—Ph | methyl | H | b5 | H |
| 2-1-108 | N—Ph | N—Ph | b1 | H | b5 | H |
| 2-1-109 | N—Ph | N—Ph | b2 | H | b5 | H |
| 2-1-110 | N—Ph | N—Ph | b3 | H | b5 | H |
| 2-1-111 | N—Ph | N—Ph | H | H | b6 | H |
| 2-1-112 | N—Ph | N—Ph | t-bu | H | b6 | H |
| 2-1-113 | N—Ph | N—Ph | methyl | H | b6 | H |
| 2-1-114 | N—Ph | N—Ph | b1 | H | b6 | H |
| 2-1-115 | N—Ph | N—Ph | b2 | H | b6 | H |
| 2-1-116 | N—Ph | N—Ph | b3 | H | b6 | H |
| 2-1-117 | N—Ph | N—Ph | H | H | b7 | H |
| 2-1-118 | N—Ph | N—Ph | t-bu | H | b7 | H |
| 2-1-119 | N—Ph | N—Ph | methyl | H | b7 | H |
| 2-1-120 | N—Ph | N—Ph | b1 | H | b7 | H |
| 2-1-121 | N—Ph | N—Ph | b2 | H | b7 | H |
| 2-1-122 | N—Ph | N—Ph | b3 | H | b7 | H |
| 2-1-123 | N—Ph | N—Ph | H | H | b8 | H |
| 2-1-124 | N—Ph | N—Ph | t-bu | H | b8 | H |
| 2-1-125 | N—Ph | N—Ph | methyl | H | b8 | H |
| 2-1-126 | N—Ph | N—Ph | b1 | H | b8 | H |
| 2-1-127 | N—Ph | N—Ph | b2 | H | b8 | H |
| 2-1-128 | N—Ph | N—Ph | b3 | H | b8 | H |
| 2-1-129 | N—Ph | N—Ph | H | H | b9 | H |
| 2-1-130 | N—Ph | N—Ph | t-bu | H | b9 | H |
| 2-1-131 | N—Ph | N—Ph | methyl | H | b9 | H |
| 2-1-132 | N—Ph | N—Ph | b1 | H | b9 | H |
| 2-1-133 | N—Ph | N—Ph | b2 | H | b9 | H |
| 2-1-134 | N—Ph | N—Ph | b3 | H | b9 | H |
| 2-1-135 | N—Ph | N—Ph | H | H | b10 | H |
| 2-1-136 | N—Ph | N—Ph | t-bu | H | b10 | H |
| 2-1-137 | N—Ph | N—Ph | methyl | H | b10 | H |
| 2-1-138 | N—Ph | N—Ph | b1 | H | b10 | H |
| 2-1-139 | N—Ph | N—Ph | b2 | H | b10 | H |
| 2-1-140 | N—Ph | N—Ph | b3 | H | b10 | H |
| 2-1-141 | N—Ph | N—Ph | H | H | a1 | a1 |
| 2-1-142 | N—Ph | N—Ph | t-bu | H | a1 | a1 |
| 2-1-143 | N—Ph | N—Ph | methyl | H | a1 | a1 |
| 2-1-144 | N—Ph | N—Ph | b1 | H | a1 | a1 |
| 2-1-145 | N—Ph | N—Ph | b2 | H | a1 | a1 |
| 2-1-146 | N—Ph | N—Ph | b3 | H | a1 | a1 |
| 2-1-147 | N—Ph | N—Ph | H | H | a2 | a2 |
| 2-1-148 | N—Ph | N—Ph | t-bu | H | a2 | a2 |
| 2-1-149 | N—Ph | N—Ph | methyl | H | a2 | a2 |
| 2-1-150 | N—Ph | N—Ph | b1 | H | a2 | a2 |
| 2-1-151 | N—Ph | N—Ph | b2 | H | a2 | a2 |
| 2-1-152 | N—Ph | N—Ph | b3 | H | a2 | a2 |
| 2-1-153 | N—Ph | N—Ph | H | H | a3 | a3 |
| 2-1-154 | N—Ph | N—Ph | t-bu | H | a3 | a3 |
| 2-1-155 | N—Ph | N—Ph | methyl | H | a3 | a3 |
| 2-1-156 | N—Ph | N—Ph | b1 | H | a3 | a3 |
| 2-1-157 | N—Ph | N—Ph | b2 | H | a3 | a3 |
| 2-1-158 | N—Ph | N—Ph | b3 | H | a3 | a3 |
| 2-1-159 | N—Ph | N—Ph | H | H | b1 | b1 |
| 2-1-160 | N—Ph | N—Ph | t-bu | H | b1 | b1 |
| 2-1-161 | N—Ph | N—Ph | methyl | H | b1 | b1 |
| 2-1-162 | N—Ph | N—Ph | b1 | H | b1 | b1 |
| 2-1-163 | N—Ph | N—Ph | b2 | H | b1 | b1 |
| 2-1-164 | N—Ph | N—Ph | b3 | H | b1 | b1 |
| 2-1-165 | N—Ph | N—Ph | H | H | b2 | b2 |
| 2-1-166 | N—Ph | N—Ph | t-bu | H | b2 | b2 |
| 2-1-167 | N—Ph | N—Ph | methyl | H | b2 | b2 |
| 2-1-168 | N—Ph | N—Ph | b1 | H | b2 | b2 |
| 2-1-169 | N—Ph | N—Ph | b2 | H | b2 | b2 |
| 2-1-170 | N—Ph | N—Ph | b3 | H | b2 | b2 |
| 2-1-171 | N—Ph | N—Ph | H | H | b3 | b3 |
| 2-1-172 | N—Ph | N—Ph | t-bu | H | b3 | b3 |
| 2-1-173 | N—Ph | N—Ph | methyl | H | b3 | b3 |
| 2-1-174 | N—Ph | N—Ph | b1 | H | b3 | b3 |
| 2-1-175 | N—Ph | N—Ph | b2 | H | b3 | b3 |
| 2-1-176 | N—Ph | N—Ph | b3 | H | b3 | b3 |
| 2-1-177 | N—Ph | N—Ph | H | H | b5 | b5 |
| 2-1-178 | N—Ph | N—Ph | t-bu | H | b5 | b5 |
| 2-1-179 | N—Ph | N—Ph | methyl | H | b5 | b5 |
| 2-1-180 | N—Ph | N—Ph | b1 | H | b5 | b5 |
| 2-1-181 | N—Ph | N—Ph | b2 | H | b5 | b5 |
| 2-1-182 | N—Ph | N—Ph | b3 | H | b5 | b5 |
| 2-1-183 | N—Ph | N—Ph | H | H | b6 | b6 |
| 2-1-184 | N—Ph | N—Ph | t-bu | H | b6 | b6 |
| 2-1-185 | N—Ph | N—Ph | methyl | H | b6 | b6 |
| 2-1-186 | N—Ph | N—Ph | b1 | H | b6 | b6 |
| 2-1-187 | N—Ph | N—Ph | b2 | H | b6 | b6 |
| 2-1-188 | N—Ph | N—Ph | b3 | H | b6 | b6 |
| 2-1-189 | N—Ph | N—Ph | H | H | b7 | b7 |
| 2-1-190 | N—Ph | N—Ph | t-bu | H | b7 | b7 |
| 2-1-191 | N—Ph | N—Ph | methyl | H | b7 | b7 |
| 2-1-192 | N—Ph | N—Ph | b1 | H | b7 | b7 |
| 2-1-193 | N—Ph | N—Ph | b2 | H | b7 | b7 |
| 2-1-194 | N—Ph | N—Ph | b3 | H | b7 | b7 |
| 2-1-195 | N-tolyl | N-tolyl | H | H | H | H |
| 2-1-196 | N-tolyl | N-tolyl | t-bu | H | H | H |
| 2-1-197 | N-tolyl | N-tolyl | methyl | H | H | H |
| 2-1-198 | N-tolyl | N-tolyl | t-bu | t-bu | t-bu | t-bu |
| 2-1-199 | N-tolyl | N-tolyl | methyl | methyl | methyl | methyl |
| 2-1-200 | N-tolyl | N-tolyl | b1 | H | H | H |
| 2-1-201 | N-tolyl | N-tolyl | b2 | H | H | H |
| 2-1-202 | N-tolyl | N-tolyl | b3 | H | H | H |
| 2-1-203 | N-tolyl | N-tolyl | H | H | H | a1 |
| 2-1-204 | N-tolyl | N-tolyl | t-bu | H | H | a1 |
| 2-1-205 | N-tolyl | N-tolyl | methyl | H | H | a1 |
| 2-1-206 | N-tolyl | N-tolyl | b1 | H | H | a1 |
| 2-1-207 | N-tolyl | N-tolyl | b2 | H | H | a1 |
| 2-1-208 | N-tolyl | N-tolyl | b3 | H | H | a1 |
| 2-1-209 | N-tolyl | N-tolyl | H | H | H | a2 |
| 2-1-210 | N-tolyl | N-tolyl | t-bu | H | H | a2 |
| 2-1-211 | N-tolyl | N-tolyl | methyl | H | H | a2 |
| 2-1-212 | N-tolyl | N-tolyl | b1 | H | H | a2 |
| 2-1-213 | N-tolyl | N-tolyl | b2 | H | H | a2 |
| 2-1-214 | N-tolyl | N-tolyl | b3 | H | H | a2 |
| 2-1-215 | N-tolyl | N-tolyl | H | H | H | a3 |
| 2-1-216 | N-tolyl | N-tolyl | t-bu | H | H | a3 |
| 2-1-217 | N-tolyl | N-tolyl | methyl | H | H | a3 |
| 2-1-218 | N-tolyl | N-tolyl | b1 | H | H | a3 |
| 2-1-219 | N-tolyl | N-tolyl | b2 | H | H | a3 |
| 2-1-220 | N-tolyl | N-tolyl | b3 | H | H | a3 |
| 2-1-221 | N-tolyl | N-tolyl | H | H | H | a4 |
| 2-1-222 | N-tolyl | N-tolyl | t-bu | H | H | a4 |
| 2-1-223 | N-tolyl | N-tolyl | methyl | H | H | a4 |
| 2-1-224 | N-tolyl | N-tolyl | b1 | H | H | a4 |
| 2-1-225 | N-tolyl | N-tolyl | b2 | H | H | a4 |
| 2-1-226 | N-tolyl | N-tolyl | b3 | H | H | a4 |
| 2-1-227 | N-tolyl | N-tolyl | H | H | H | a5 |
| 2-1-228 | N-tolyl | N-tolyl | t-bu | H | H | a5 |
| 2-1-229 | N-tolyl | N-tolyl | methyl | H | H | a5 |
| 2-1-230 | N-tolyl | N-tolyl | b1 | H | H | a5 |
| 2-1-231 | N-tolyl | N-tolyl | b2 | H | H | a5 |
| 2-1-232 | N-tolyl | N-tolyl | b3 | H | H | a5 |
| 2-1-233 | N-tolyl | N-tolyl | H | H | H | a6 |
| 2-1-234 | N-tolyl | N-tolyl | t-bu | H | H | a6 |
| 2-1-235 | N-tolyl | N-tolyl | methyl | H | H | a6 |
| 2-1-236 | N-tolyl | N-tolyl | b1 | H | H | a6 |
| 2-1-237 | N-tolyl | N-tolyl | b2 | H | H | a6 |
| 2-1-238 | N-tolyl | N-tolyl | b3 | H | H | a6 |
| 2-1-239 | N-tolyl | N-tolyl | H | H | a1 | H |
| 2-1-240 | N-tolyl | N-tolyl | t-bu | H | a1 | H |
| 2-1-241 | N-tolyl | N-tolyl | methyl | H | a1 | H |
| 2-1-242 | N-tolyl | N-tolyl | b1 | H | a1 | H |
| 2-1-243 | N-tolyl | N-tolyl | b2 | H | a1 | H |
| 2-1-244 | N-tolyl | N-tolyl | b3 | H | a1 | H |
| 2-1-245 | N-tolyl | N-tolyl | H | H | a2 | H |
| 2-1-246 | N-tolyl | N-tolyl | t-bu | H | a2 | H |
| 2-1-247 | N-tolyl | N-tolyl | methyl | H | a2 | H |
| 2-1-248 | N-tolyl | N-tolyl | b1 | H | a2 | H |
| 2-1-249 | N-tolyl | N-tolyl | b2 | H | a2 | H |
| 2-1-250 | N-tolyl | N-tolyl | b3 | H | a2 | H |
| 2-1-251 | N-tolyl | N-tolyl | H | H | a3 | H |
| 2-1-252 | N-tolyl | N-tolyl | t-bu | H | a3 | H |
| 2-1-253 | N-tolyl | N-tolyl | methyl | H | a3 | H |
| 2-1-254 | N-tolyl | N-tolyl | b1 | H | a3 | H |
| 2-1-255 | N-tolyl | N-tolyl | b2 | H | a3 | H |
| 2-1-256 | N-tolyl | N-tolyl | b3 | H | a3 | H |
| 2-1-257 | N-tolyl | N-tolyl | H | H | a4 | H |
| 2-1-258 | N-tolyl | N-tolyl | t-bu | H | a4 | H |
| 2-1-259 | N-tolyl | N-tolyl | methyl | H | a4 | H |
| 2-1-260 | N-tolyl | N-tolyl | b1 | H | a4 | H |
| 2-1-261 | N-tolyl | N-tolyl | b2 | H | a4 | H |
| 2-1-262 | N-tolyl | N-tolyl | b3 | H | a4 | H |
| 2-1-263 | N-tolyl | N-tolyl | H | H | a5 | H |
| 2-1-264 | N-tolyl | N-tolyl | t-bu | H | a5 | H |
| 2-1-265 | N-tolyl | N-tolyl | methyl | H | a5 | H |
| 2-1-266 | N-tolyl | N-tolyl | b1 | H | a5 | H |
| 2-1-267 | N-tolyl | N-tolyl | b2 | H | a5 | H |
| 2-1-268 | N-tolyl | N-tolyl | b3 | H | a5 | H |
| 2-1-269 | N-tolyl | N-tolyl | H | H | a6 | H |
| 2-1-270 | N-tolyl | N-tolyl | t-bu | H | a6 | H |
| 2-1-271 | N-tolyl | N-tolyl | methyl | H | a6 | H |
| 2-1-272 | N-tolyl | N-tolyl | b1 | H | a6 | H |
| 2-1-273 | N-tolyl | N-tolyl | b2 | H | a6 | H |
| 2-1-274 | N-tolyl | N-tolyl | b3 | H | a6 | H |
| 2-1-275 | N-tolyl | N-tolyl | H | H | b1 | H |
| 2-1-276 | N-tolyl | N-tolyl | t-bu | H | b1 | H |
| 2-1-277 | N-tolyl | N-tolyl | methyl | H | b1 | H |
| 2-1-278 | N-tolyl | N-tolyl | b1 | H | b1 | H |
| 2-1-279 | N-tolyl | N-tolyl | b2 | H | b1 | H |
| 2-1-280 | N-tolyl | N-tolyl | b3 | H | b1 | H |
| 2-1-281 | N-tolyl | N-tolyl | H | H | b2 | H |
| 2-1-282 | N-tolyl | N-tolyl | t-bu | H | b2 | H |
| 2-1-283 | N-tolyl | N-tolyl | methyl | H | b2 | H |
| 2-1-284 | N-tolyl | N-tolyl | b1 | H | b2 | H |
| 2-1-285 | N-tolyl | N-tolyl | b2 | H | b2 | H |
| 2-1-286 | N-tolyl | N-tolyl | b3 | H | b2 | H |
| 2-1-287 | N-tolyl | N-tolyl | H | H | b3 | H |
| 2-1-288 | N-tolyl | N-tolyl | t-bu | H | b3 | H |
| 2-1-289 | N-tolyl | N-tolyl | methyl | H | b3 | H |
| 2-1-290 | N-tolyl | N-tolyl | b1 | H | b3 | H |
| 2-1-291 | N-tolyl | N-tolyl | b2 | H | b3 | H |
| 2-1-292 | N-tolyl | N-tolyl | b3 | H | b3 | H |
| 2-1-293 | N-tolyl | N-tolyl | H | H | b4 | H |
| 2-1-294 | N-tolyl | N-tolyl | t-bu | H | b4 | H |
| 2-1-295 | N-tolyl | N-tolyl | methyl | H | b4 | H |
| 2-1-296 | N-tolyl | N-tolyl | b1 | H | b4 | H |
| 2-1-297 | N-tolyl | N-tolyl | b2 | H | b4 | H |
| 2-1-298 | N-tolyl | N-tolyl | b3 | H | b4 | H |
| 2-1-299 | N-tolyl | N-tolyl | H | H | b5 | H |
| 2-1-300 | N-tolyl | N-tolyl | t-bu | H | b5 | H |
| 2-1-301 | N-tolyl | N-tolyl | methyl | H | b5 | H |
| 2-1-302 | N-tolyl | N-tolyl | b1 | H | b5 | H |
| 2-1-303 | N-tolyl | N-tolyl | b2 | H | b5 | H |
| 2-1-304 | N-tolyl | N-tolyl | b3 | H | b5 | H |
| 2-1-305 | N-tolyl | N-tolyl | H | H | b6 | H |
| 2-1-306 | N-tolyl | N-tolyl | t-bu | H | b6 | H |
| 2-1-307 | N-tolyl | N-tolyl | methyl | H | b6 | H |
| 2-1-308 | N-tolyl | N-tolyl | b1 | H | b6 | H |
| 2-1-309 | N-tolyl | N-tolyl | b2 | H | b6 | H |
| 2-1-310 | N-tolyl | N-tolyl | b3 | H | b6 | H |
| 2-1-311 | N-tolyl | N-tolyl | H | H | b7 | H |
| 2-1-312 | N-tolyl | N-tolyl | t-bu | H | b7 | H |
| 2-1-313 | N-tolyl | N-tolyl | methyl | H | b7 | H |
| 2-1-314 | N-tolyl | N-tolyl | b1 | H | b7 | H |
| 2-1-315 | N-tolyl | N-tolyl | b2 | H | b7 | H |
| 2-1-316 | N-tolyl | N-tolyl | b3 | H | b7 | H |
| 2-1-317 | N-tolyl | N-tolyl | H | H | b8 | H |
| 2-1-318 | N-tolyl | N-tolyl | t-bu | H | b8 | H |
| 2-1-319 | N-tolyl | N-tolyl | methyl | H | b8 | H |
| 2-1-320 | N-tolyl | N-tolyl | b1 | H | b8 | H |
| 2-1-321 | N-tolyl | N-tolyl | b2 | H | b8 | H |
| 2-1-322 | N-tolyl | N-tolyl | b3 | H | b8 | H |
| 2-1-323 | N-tolyl | N-tolyl | H | H | b9 | H |
| 2-1-324 | N-tolyl | N-tolyl | t-bu | H | b9 | H |
| 2-1-325 | N-tolyl | N-tolyl | methyl | H | b9 | H |
| 2-1-326 | N-tolyl | N-tolyl | b1 | H | b9 | H |
| 2-1-327 | N-tolyl | N-tolyl | b2 | H | b9 | H |
| 2-1-328 | N-tolyl | N-tolyl | b3 | H | b9 | H |
| 2-1-329 | N-tolyl | N-tolyl | H | H | b10 | H |
| 2-1-330 | N-tolyl | N-tolyl | t-bu | H | b10 | H |
| 2-1-331 | N-tolyl | N-tolyl | methyl | H | b10 | H |
| 2-1-332 | N-tolyl | N-tolyl | b1 | H | b10 | H |
| 2-1-333 | N-tolyl | N-tolyl | b2 | H | b10 | H |
| 2-1-334 | N-tolyl | N-tolyl | b3 | H | b10 | H |
| 2-1-335 | N-tolyl | N-tolyl | H | H | a1 | a1 |
| 2-1-336 | N-tolyl | N-tolyl | t-bu | H | a1 | a1 |
| 2-1-337 | N-tolyl | N-tolyl | methyl | H | a1 | a1 |
| 2-1-338 | N-tolyl | N-tolyl | b1 | H | a1 | a1 |
| 2-1-339 | N-tolyl | N-tolyl | b2 | H | a1 | a1 |
| 2-1-340 | N-tolyl | N-tolyl | b3 | H | a1 | a1 |
| 2-1-341 | N-tolyl | N-tolyl | H | H | a2 | a2 |
| 2-1-342 | N-tolyl | N-tolyl | t-bu | H | a2 | a2 |
| 2-1-343 | N-tolyl | N-tolyl | methyl | H | a2 | a2 |
| 2-1-344 | N-tolyl | N-tolyl | b1 | H | a2 | a2 |
| 2-1-345 | N-tolyl | N-tolyl | b2 | H | a2 | a2 |
| 2-1-346 | N-tolyl | N-tolyl | b3 | H | a2 | a2 |
| 2-1-347 | N-tolyl | N-tolyl | H | H | a3 | a3 |
| 2-1-348 | N-tolyl | N-tolyl | t-bu | H | a3 | a3 |
| 2-1-349 | N-tolyl | N-tolyl | methyl | H | a3 | a3 |
| 2-1-350 | N-tolyl | N-tolyl | b1 | H | a3 | a3 |
| 2-1-351 | N-tolyl | N-tolyl | b2 | H | a3 | a3 |
| 2-1-352 | N-tolyl | N-tolyl | b3 | H | a3 | a3 |
| 2-1-353 | N-tolyl | N-tolyl | H | H | b1 | b1 |
| 2-1-354 | N-tolyl | N-tolyl | t-bu | H | b1 | b1 |
| 2-1-355 | N-tolyl | N-tolyl | methyl | H | b1 | b1 |
| 2-1-356 | N-tolyl | N-tolyl | b1 | H | b1 | b1 |
| 2-1-357 | N-tolyl | N-tolyl | b2 | H | b1 | b1 |
| 2-1-358 | N-tolyl | N-tolyl | b3 | H | b1 | b1 |
| 2-1-359 | N-tolyl | N-tolyl | H | H | b2 | b2 |
| 2-1-360 | N-tolyl | N-tolyl | t-bu | H | b2 | b2 |
| 2-1-361 | N-tolyl | N-tolyl | methyl | H | b2 | b2 |
| 2-1-362 | N-tolyl | N-tolyl | b1 | H | b2 | b2 |
| 2-1-363 | N-tolyl | N-tolyl | b2 | H | b2 | b2 |
| 2-1-364 | N-tolyl | N-tolyl | b3 | H | b2 | b2 |
| 2-1-365 | N-tolyl | N-tolyl | H | H | b3 | b3 |
| 2-1-366 | N-tolyl | N-tolyl | t-bu | H | b3 | b3 |
| 2-1-367 | N-tolyl | N-tolyl | methyl | H | b3 | b3 |
| 2-1-368 | N-tolyl | N-tolyl | b1 | H | b3 | b3 |
| 2-1-369 | N-tolyl | N-tolyl | b2 | H | b3 | b3 |
| 2-1-370 | N-tolyl | N-tolyl | b3 | H | b3 | b3 |
| 2-1-371 | N-tolyl | N-tolyl | H | H | b5 | b5 |
| 2-1-372 | N-tolyl | N-tolyl | t-bu | H | b5 | b5 |
| 2-1-373 | N-tolyl | N-tolyl | methyl | H | b5 | b5 |
| 2-1-374 | N-tolyl | N-tolyl | b1 | H | b5 | b5 |
| 2-1-375 | N-tolyl | N-tolyl | b2 | H | b5 | b5 |
| 2-1-376 | N-tolyl | N-tolyl | b3 | H | b5 | b5 |
| 2-1-377 | N-tolyl | N-tolyl | H | H | b6 | b6 |
| 2-1-378 | N-tolyl | N-tolyl | t-bu | H | b6 | b6 |
| 2-1-379 | N-tolyl | N-tolyl | methyl | H | b6 | b6 |
| 2-1-380 | N-tolyl | N-tolyl | b1 | H | b6 | b6 |
| 2-1-381 | N-tolyl | N-tolyl | b2 | H | b6 | b6 |
| 2-1-382 | N-tolyl | N-tolyl | b3 | H | b6 | b6 |
| 2-1-383 | N-tolyl | N-tolyl | H | H | b7 | b7 |
| 2-1-384 | N-tolyl | N-tolyl | t-bu | H | b7 | b7 |
| 2-1-385 | N-tolyl | N-tolyl | methyl | H | b7 | b7 |
| 2-1-386 | N-tolyl | N-tolyl | b1 | H | b7 | b7 |
| 2-1-387 | N-tolyl | N-tolyl | b2 | H | b7 | b7 |
| 2-1-388 | N-tolyl | N-tolyl | b3 | H | b7 | b7 |
| 2-1-389 | N-Biph | N-Biph | H | H | H | H |
| 2-1-390 | N-Biph | N-Biph | t-bu | H | H | H |
| 2-1-391 | N-Biph | N-Biph | methyl | H | H | H |
| 2-1-392 | N-Biph | N-Biph | t-bu | t-bu | t-bu | t-bu |
| 2-1-393 | N-Biph | N-Biph | methyl | methyl | methyl | methyl |
| 2-1-394 | N-Biph | N-Biph | b1 | H | H | H |
| 2-1-395 | N-Biph | N-Biph | b2 | H | H | H |
| 2-1-396 | N-Biph | N-Biph | b3 | H | H | H |
| 2-1-397 | N-Biph | N-Biph | H | H | H | a1 |
| 2-1-398 | N-Biph | N-Biph | t-bu | H | H | a1 |
| 2-1-399 | N-Biph | N-Biph | methyl | H | H | a1 |
| 2-1-400 | N-Biph | N-Biph | b1 | H | H | a1 |
| 2-1-401 | N-Biph | N-Biph | b2 | H | H | a1 |
| 2-1-402 | N-Biph | N-Biph | b3 | H | H | a1 |
| 2-1-403 | N-Biph | N-Biph | H | H | H | a2 |
| 2-1-404 | N-Biph | N-Biph | t-bu | H | H | a2 |
| 2-1-405 | N-Biph | N-Biph | methyl | H | H | a2 |
| 2-1-406 | N-Biph | N-Biph | b1 | H | H | a2 |
| 2-1-407 | N-Biph | N-Biph | b2 | H | H | a2 |
| 2-1-408 | N-Biph | N-Biph | b3 | H | H | a2 |
| 2-1-409 | N-Biph | N-Biph | H | H | H | a3 |
| 2-1-410 | N-Biph | N-Biph | t-bu | H | H | a3 |
| 2-1-411 | N-Biph | N-Biph | methyl | H | H | a3 |
| 2-1-412 | N-Biph | N-Biph | b1 | H | H | a3 |
| 2-1-413 | N-Biph | N-Biph | b2 | H | H | a3 |
| 2-1-414 | N-Biph | N-Biph | b3 | H | H | a3 |
| 2-1-415 | N-Biph | N-Biph | H | H | H | a4 |
| 2-1-416 | N-Biph | N-Biph | t-bu | H | H | a4 |
| 2-1-417 | N-Biph | N-Biph | methyl | H | H | a4 |
| 2-1-418 | N-Biph | N-Biph | b1 | H | H | a4 |
| 2-1-419 | N-Biph | N-Biph | b2 | H | H | a4 |
| 2-1-420 | N-Biph | N-Biph | b3 | H | H | a4 |
| 2-1-421 | N-Biph | N-Biph | H | H | H | a5 |
| 2-1-422 | N-Biph | N-Biph | t-bu | H | H | a5 |
| 2-1-423 | N-Biph | N-Biph | methyl | H | H | a5 |
| 2-1-424 | N-Biph | N-Biph | b1 | H | H | a5 |
| 2-1-425 | N-Biph | N-Biph | b2 | H | H | a5 |
| 2-1-426 | N-Biph | N-Biph | b3 | H | H | a5 |
| 2-1-427 | N-Biph | N-Biph | H | H | H | a6 |
| 2-1-428 | N-Biph | N-Biph | t-bu | H | H | a6 |
| 2-1-429 | N-Biph | N-Biph | methyl | H | H | a6 |
| 2-1-430 | N-Biph | N-Biph | b1 | H | H | a6 |
| 2-1-431 | N-Biph | N-Biph | b2 | H | H | a6 |
| 2-1-432 | N-Biph | N-Biph | b3 | H | H | a6 |
| 2-1-433 | N-Biph | N-Biph | H | H | a1 | H |
| 2-1-434 | N-Biph | N-Biph | t-bu | H | a1 | H |
| 2-1-435 | N-Biph | N-Biph | methyl | H | a1 | H |
| 2-1-436 | N-Biph | N-Biph | b1 | H | a1 | H |
| 2-1-437 | N-Biph | N-Biph | b2 | H | a1 | H |
| 2-1-438 | N-Biph | N-Biph | b3 | H | a1 | H |
| 2-1-439 | N-Biph | N-Biph | H | H | a2 | H |
| 2-1-440 | N-Biph | N-Biph | t-bu | H | a2 | H |
| 2-1-441 | N-Biph | N-Biph | methyl | H | a2 | H |
| 2-1-442 | N-Biph | N-Biph | b1 | H | a2 | H |
| 2-1-443 | N-Biph | N-Biph | b2 | H | a2 | H |
| 2-1-444 | N-Biph | N-Biph | b3 | H | a2 | H |
| 2-1-445 | N-Biph | N-Biph | H | H | a3 | H |
| 2-1-446 | N-Biph | N-Biph | t-bu | H | a3 | H |
| 2-1-447 | N-Biph | N-Biph | methyl | H | a3 | H |
| 2-1-448 | N-Biph | N-Biph | b1 | H | a3 | H |
| 2-1-449 | N-Biph | N-Biph | b2 | H | a3 | H |
| 2-1-450 | N-Biph | N-Biph | b3 | H | a3 | H |
| 2-1-451 | N-Biph | N-Biph | H | H | a4 | H |
| 2-1-452 | N-Biph | N-Biph | t-bu | H | a4 | H |
| 2-1-453 | N-Biph | N-Biph | methyl | H | a4 | H |
| 2-1-454 | N-Biph | N-Biph | b1 | H | a4 | H |
| 2-1-455 | N-Biph | N-Biph | b2 | H | a4 | H |
| 2-1-456 | N-Biph | N-Biph | b3 | H | a4 | H |
| 2-1-457 | N-Biph | N-Biph | H | H | a5 | H |
| 2-1-458 | N-Biph | N-Biph | t-bu | H | a5 | H |
| 2-1-459 | N-Biph | N-Biph | methyl | H | a5 | H |
| 2-1-460 | N-Biph | N-Biph | b1 | H | a5 | H |
| 2-1-461 | N-Biph | N-Biph | b2 | H | a5 | H |
| 2-1-462 | N-Biph | N-Biph | b3 | H | a5 | H |
| 2-1-463 | N-Biph | N-Biph | H | H | a6 | H |
| 2-1-464 | N-Biph | N-Biph | t-bu | H | a6 | H |
| 2-1-465 | N-Biph | N-Biph | methyl | H | a6 | H |
| 2-1-466 | N-Biph | N-Biph | b1 | H | a6 | H |
| 2-1-467 | N-Biph | N-Biph | b2 | H | a6 | H |
| 2-1-468 | N-Biph | N-Biph | b3 | H | a6 | H |
| 2-1-469 | N-Biph | N-Biph | H | H | b1 | H |
| 2-1-470 | N-Biph | N-Biph | t-bu | H | b1 | H |
| 2-1-471 | N-Biph | N-Biph | methyl | H | b1 | H |
| 2-1-472 | N-Biph | N-Biph | b1 | H | b1 | H |
| 2-1-473 | N-Biph | N-Biph | b2 | H | b1 | H |
| 2-1-474 | N-Biph | N-Biph | b3 | H | b1 | H |
| 2-1-475 | N-Biph | N-Biph | H | H | b2 | H |
| 2-1-476 | N-Biph | N-Biph | t-bu | H | b2 | H |
| 2-1-477 | N-Biph | N-Biph | methyl | H | b2 | H |
| 2-1-478 | N-Biph | N-Biph | b1 | H | b2 | H |
| 2-1-479 | N-Biph | N-Biph | b2 | H | b2 | H |
| 2-1-480 | N-Biph | N-Biph | b3 | H | b2 | H |
| 2-1-481 | N-Biph | N-Biph | H | H | b3 | H |
| 2-1-482 | N-Biph | N-Biph | t-bu | H | b3 | H |
| 2-1-483 | N-Biph | N-Biph | methyl | H | b3 | H |
| 2-1-484 | N-Biph | N-Biph | b1 | H | b3 | H |
| 2-1-485 | N-Biph | N-Biph | b2 | H | b3 | H |
| 2-1-486 | N-Biph | N-Biph | b3 | H | b3 | H |
| 2-1-487 | N-Biph | N-Biph | H | H | b4 | H |
| 2-1-488 | N-Biph | N-Biph | t-bu | H | b4 | H |
| 2-1-489 | N-Biph | N-Biph | methyl | H | b4 | H |
| 2-1-490 | N-Biph | N-Biph | b1 | H | b4 | H |
| 2-1-491 | N-Biph | N-Biph | b2 | H | b4 | H |
| 2-1-492 | N-Biph | N-Biph | b3 | H | b4 | H |
| 2-1-493 | N-Biph | N-Biph | H | H | b5 | H |
| 2-1-494 | N-Biph | N-Biph | t-bu | H | b5 | H |
| 2-1-495 | N-Biph | N-Biph | methyl | H | b5 | H |
| 2-1-496 | N-Biph | N-Biph | b1 | H | b5 | H |
| 2-1-497 | N-Biph | N-Biph | b2 | H | b5 | H |
| 2-1-498 | N-Biph | N-Biph | b3 | H | b5 | H |
| 2-1-499 | N-Biph | N-Biph | H | H | b6 | H |
| 2-1-500 | N-Biph | N-Biph | t-bu | H | b6 | H |
| 2-1-501 | N-Biph | N-Biph | methyl | H | b6 | H |
| 2-1-502 | N-Biph | N-Biph | b1 | H | b6 | H |
| 2-1-503 | N-Biph | N-Biph | b2 | H | b6 | H |
| 2-1-504 | N-Biph | N-Biph | b3 | H | b6 | H |
| 2-1-505 | N-Biph | N-Biph | H | H | b7 | H |
| 2-1-506 | N-Biph | N-Biph | t-bu | H | b7 | H |
| 2-1-507 | N-Biph | N-Biph | methyl | H | b7 | H |
| 2-1-508 | N-Biph | N-Biph | b1 | H | b7 | H |
| 2-1-509 | N-Biph | N-Biph | b2 | H | b7 | H |
| 2-1-510 | N-Biph | N-Biph | b3 | H | b7 | H |
| 2-1-511 | N-Biph | N-Biph | H | H | b8 | H |
| 2-1-512 | N-Biph | N-Biph | t-bu | H | b8 | H |
| 2-1-513 | N-Biph | N-Biph | methyl | H | b8 | H |
| 2-1-514 | N-Biph | N-Biph | b1 | H | b8 | H |
| 2-1-515 | N-Biph | N-Biph | b2 | H | b8 | H |
| 2-1-516 | N-Biph | N-Biph | b3 | H | b8 | H |
| 2-1-517 | N-Biph | N-Biph | H | H | b9 | H |
| 2-1-518 | N-Biph | N-Biph | t-bu | H | b9 | H |
| 2-1-519 | N-Biph | N-Biph | methyl | H | b9 | H |
| 2-1-520 | N-Biph | N-Biph | b1 | H | b9 | H |
| 2-1-521 | N-Biph | N-Biph | b2 | H | b9 | H |
| 2-1-522 | N-Biph | N-Biph | b3 | H | b9 | H |
| 2-1-523 | N-Biph | N-Biph | H | H | b10 | H |
| 2-1-524 | N-Biph | N-Biph | t-bu | H | b10 | H |
| 2-1-525 | N-Biph | N-Biph | methyl | H | b10 | H |
| 2-1-526 | N-Biph | N-Biph | b1 | H | b10 | H |
| 2-1-527 | N-Biph | N-Biph | b2 | H | b10 | H |
| 2-1-528 | N-Biph | N-Biph | b3 | H | b10 | H |
| 2-1-529 | N-Biph | N-Biph | H | H | a1 | a1 |
| 2-1-530 | N-Biph | N-Biph | t-bu | H | a1 | a1 |
| 2-1-531 | N-Biph | N-Biph | methyl | H | a1 | a1 |
| 2-1-532 | N-Biph | N-Biph | b1 | H | a1 | a1 |
| 2-1-533 | N-Biph | N-Biph | b2 | H | a1 | a1 |
| 2-1-534 | N-Biph | N-Biph | b3 | H | a1 | a1 |
| 2-1-535 | N-Biph | N-Biph | H | H | a2 | a2 |
| 2-1-536 | N-Biph | N-Biph | t-bu | H | a2 | a2 |
| 2-1-537 | N-Biph | N-Biph | methyl | H | a2 | a2 |
| 2-1-538 | N-Biph | N-Biph | b1 | H | a2 | a2 |
| 2-1-539 | N-Biph | N-Biph | b2 | H | a2 | a2 |
| 2-1-540 | N-Biph | N-Biph | b3 | H | a2 | a2 |
| 2-1-541 | N-Biph | N-Biph | H | H | a3 | a3 |
| 2-1-542 | N-Biph | N-Biph | t-bu | H | a3 | a3 |
| 2-1-543 | N-Biph | N-Biph | methyl | H | a3 | a3 |
| 2-1-544 | N-Biph | N-Biph | b1 | H | a3 | a3 |
| 2-1-545 | N-Biph | N-Biph | b2 | H | a3 | a3 |
| 2-1-546 | N-Biph | N-Biph | b3 | H | a3 | a3 |
| 2-1-547 | N-Biph | N-Biph | H | H | b1 | b1 |
| 2-1-548 | N-Biph | N-Biph | t-bu | H | b1 | b1 |
| 2-1-549 | N-Biph | N-Biph | methyl | H | b1 | b1 |
| 2-1-550 | N-Biph | N-Biph | b1 | H | b1 | b1 |
| 2-1-551 | N-Biph | N-Biph | b2 | H | b1 | b1 |
| 2-1-552 | N-Biph | N-Biph | b3 | H | b1 | b1 |
| 2-1-553 | N-Biph | N-Biph | H | H | b2 | b2 |
| 2-1-554 | N-Biph | N-Biph | t-bu | H | b2 | b2 |
| 2-1-555 | N-Biph | N-Biph | methyl | H | b2 | b2 |
| 2-1-556 | N-Biph | N-Biph | b1 | H | b2 | b2 |
| 2-1-557 | N-Biph | N-Biph | b2 | H | b2 | b2 |
| 2-1-558 | N-Biph | N-Biph | b3 | H | b2 | b2 |
| 2-1-559 | N-Biph | N-Biph | H | H | b3 | b3 |
| 2-1-560 | N-Biph | N-Biph | t-bu | H | b3 | b3 |
| 2-1-561 | N-Biph | N-Biph | methyl | H | b3 | b3 |
| 2-1-562 | N-Biph | N-Biph | b1 | H | b3 | b3 |
| 2-1-563 | N-Biph | N-Biph | b2 | H | b3 | b3 |
| 2-1-564 | N-Biph | N-Biph | b3 | H | b3 | b3 |
| 2-1-565 | N-Biph | N-Biph | H | H | b5 | b5 |
| 2-1-566 | N-Biph | N-Biph | t-bu | H | b5 | b5 |
| 2-1-567 | N-Biph | N-Biph | methyl | H | b5 | b5 |
| 2-1-568 | N-Biph | N-Biph | b1 | H | b5 | b5 |
| 2-1-569 | N-Biph | N-Biph | b2 | H | b5 | b5 |
| 2-1-570 | N-Biph | N-Biph | b3 | H | b5 | b5 |
| 2-1-571 | N-Biph | N-Biph | H | H | b6 | b6 |
| 2-1-572 | N-Biph | N-Biph | t-bu | H | b6 | b6 |
| 2-1-573 | N-Biph | N-Biph | methyl | H | b6 | b6 |
| 2-1-574 | N-Biph | N-Biph | b1 | H | b6 | b6 |
| 2-1-575 | N-Biph | N-Biph | b2 | H | b6 | b6 |
| 2-1-576 | N-Biph | N-Biph | b3 | H | b6 | b6 |
| 2-1-577 | N-Biph | N-Biph | H | H | b7 | b7 |
| 2-1-578 | N-Biph | N-Biph | t-bu | H | b7 | b7 |
| 2-1-579 | N-Biph | N-Biph | methyl | H | b7 | b7 |
| 2-1-580 | N-Biph | N-Biph | b1 | H | b7 | b7 |
| 2-1-581 | N-Biph | N-Biph | b2 | H | b7 | b7 |
| 2-1-582 | N-Biph | N-Biph | b3 | H | b7 | b7 |
| 2-1-583 | O | N—Ph | H | H | H | H |
| 2-1-584 | O | N—Ph | t-bu | H | H | H |
| 2-1-585 | O | N—Ph | methyl | H | H | H |
| 2-1-586 | O | N—Ph | t-bu | t-bu | t-bu | t-bu |
| 2-1-587 | O | N—Ph | methyl | methyl | methyl | methyl |
| 2-1-588 | O | N—Ph | b1 | H | H | H |
| 2-1-589 | O | N—Ph | b2 | H | H | H |
| 2-1-590 | O | N—Ph | b3 | H | H | H |
| 2-1-591 | O | N—Ph | H | H | H | a1 |
| 2-1-592 | O | N—Ph | t-bu | H | H | a1 |
| 2-1-593 | O | N—Ph | methyl | H | H | a1 |
| 2-1-594 | O | N—Ph | b1 | H | H | a1 |
| 2-1-595 | O | N—Ph | b2 | H | H | a1 |
| 2-1-596 | O | N—Ph | b3 | H | H | a1 |
| 2-1-597 | O | N—Ph | H | H | H | a2 |
| 2-1-598 | O | N—Ph | t-bu | H | H | a2 |
| 2-1-599 | O | N—Ph | methyl | H | H | a2 |
| 2-1-600 | O | N—Ph | b1 | H | H | a2 |
| 2-1-601 | O | N—Ph | b2 | H | H | a2 |
| 2-1-602 | O | N—Ph | b3 | H | H | a2 |
| 2-1-603 | O | N—Ph | H | H | H | a3 |
| 2-1-604 | O | N—Ph | t-bu | H | H | a3 |
| 2-1-605 | O | N—Ph | methyl | H | H | a3 |
| 2-1-606 | O | N—Ph | b1 | H | H | a3 |
| 2-1-607 | O | N—Ph | b2 | H | H | a3 |
| 2-1-608 | O | N—Ph | b3 | H | H | a3 |
| 2-1-609 | O | N—Ph | H | H | H | a4 |
| 2-1-610 | O | N—Ph | t-bu | H | H | a4 |
| 2-1-611 | O | N—Ph | methyl | H | H | a4 |
| 2-1-612 | O | N—Ph | b1 | H | H | a4 |
| 2-1-613 | O | N—Ph | b2 | H | H | a4 |
| 2-1-614 | O | N—Ph | b3 | H | H | a4 |
| 2-1-615 | O | N—Ph | H | H | H | a5 |
| 2-1-616 | O | N—Ph | t-bu | H | H | a5 |
| 2-1-617 | O | N—Ph | methyl | H | H | a5 |
| 2-1-618 | O | N—Ph | b1 | H | H | a5 |
| 2-1-619 | O | N—Ph | b2 | H | H | a5 |
| 2-1-620 | O | N—Ph | b3 | H | H | a5 |
| 2-1-621 | O | N—Ph | H | H | H | a6 |
| 2-1-622 | O | N—Ph | t-bu | H | H | a6 |
| 2-1-623 | O | N—Ph | methyl | H | H | a6 |
| 2-1-624 | O | N—Ph | b1 | H | H | a6 |
| 2-1-625 | O | N—Ph | b2 | H | H | a6 |
| 2-1-626 | O | N—Ph | b3 | H | H | a6 |
| 2-1-627 | O | N—Ph | H | H | a1 | H |
| 2-1-628 | O | N—Ph | t-bu | H | a1 | H |
| 2-1-629 | O | N—Ph | methyl | H | a1 | H |
| 2-1-630 | O | N—Ph | b1 | H | a1 | H |
| 2-1-631 | O | N—Ph | b2 | H | a1 | H |
| 2-1-632 | O | N—Ph | b3 | H | a1 | H |
| 2-1-633 | O | N—Ph | H | H | a2 | H |
| 2-1-634 | O | N—Ph | t-bu | H | a2 | H |
| 2-1-635 | O | N—Ph | methyl | H | a2 | H |
| 2-1-636 | O | N—Ph | b1 | H | a2 | H |
| 2-1-637 | O | N—Ph | b2 | H | a2 | H |
| 2-1-638 | O | N—Ph | b3 | H | a2 | H |
| 2-1-639 | O | N—Ph | H | H | a3 | H |
| 2-1-640 | O | N—Ph | t-bu | H | a3 | H |
| 2-1-641 | O | N—Ph | methyl | H | a3 | H |
| 2-1-642 | O | N—Ph | b1 | H | a3 | H |
| 2-1-643 | O | N—Ph | b2 | H | a3 | H |
| 2-1-644 | O | N—Ph | b3 | H | a3 | H |
| 2-1-645 | O | N—Ph | H | H | a4 | H |
| 2-1-646 | O | N—Ph | t-bu | H | a4 | H |
| 2-1-647 | O | N—Ph | methyl | H | a4 | H |
| 2-1-648 | O | N—Ph | b1 | H | a4 | H |
| 2-1-649 | O | N—Ph | b2 | H | a4 | H |
| 2-1-650 | O | N—Ph | b3 | H | a4 | H |
| 2-1-651 | O | N—Ph | H | H | a5 | H |
| 2-1-652 | O | N—Ph | t-bu | H | a5 | H |
| 2-1-653 | O | N—Ph | methyl | H | a5 | H |
| 2-1-654 | O | N—Ph | b1 | H | a5 | H |
| 2-1-655 | O | N—Ph | b2 | H | a5 | H |
| 2-1-656 | O | N—Ph | b3 | H | a5 | H |
| 2-1-657 | O | N—Ph | H | H | a6 | H |
| 2-1-658 | O | N—Ph | t-bu | H | a6 | H |
| 2-1-659 | O | N—Ph | methyl | H | a6 | H |
| 2-1-660 | O | N—Ph | b1 | H | a6 | H |
| 2-1-661 | O | N—Ph | b2 | H | a6 | H |
| 2-1-662 | O | N—Ph | b3 | H | a6 | H |
| 2-1-663 | O | N—Ph | H | H | b1 | H |
| 2-1-664 | O | N—Ph | t-bu | H | b1 | H |
| 2-1-665 | O | N—Ph | methyl | H | b1 | H |
| 2-1-666 | O | N—Ph | b1 | H | b1 | H |
| 2-1-667 | O | N—Ph | b2 | H | b1 | H |
| 2-1-668 | O | N—Ph | b3 | H | b1 | H |
| 2-1-669 | O | N—Ph | H | H | b2 | H |
| 2-1-670 | O | N—Ph | t-bu | H | b2 | H |
| 2-1-671 | O | N—Ph | methyl | H | b2 | H |
| 2-1-672 | O | N—Ph | b1 | H | b2 | H |
| 2-1-673 | O | N—Ph | b2 | H | b2 | H |
| 2-1-674 | O | N—Ph | b3 | H | b2 | H |
| 2-1-675 | O | N—Ph | H | H | b3 | H |
| 2-1-676 | O | N—Ph | t-bu | H | b3 | H |
| 2-1-677 | O | N—Ph | methyl | H | b3 | H |
| 2-1-678 | O | N—Ph | b1 | H | b3 | H |
| 2-1-679 | O | N—Ph | b2 | H | b3 | H |
| 2-1-680 | O | N—Ph | b3 | H | b3 | H |
| 2-1-681 | O | N—Ph | H | H | b4 | H |
| 2-1-682 | O | N—Ph | t-bu | H | b4 | H |
| 2-1-683 | O | N—Ph | methyl | H | b4 | H |
| 2-1-684 | O | N—Ph | b1 | H | b4 | H |
| 2-1-685 | O | N—Ph | b2 | H | b4 | H |
| 2-1-686 | O | N—Ph | b3 | H | b4 | H |
| 2-1-687 | O | N—Ph | H | H | b5 | H |
| 2-1-688 | O | N—Ph | t-bu | H | b5 | H |
| 2-1-689 | O | N—Ph | methyl | H | b5 | H |
| 2-1-690 | O | N—Ph | b1 | H | b5 | H |
| 2-1-691 | O | N—Ph | b2 | H | b5 | H |
| 2-1-692 | O | N—Ph | b3 | H | b5 | H |
| 2-1-693 | O | N—Ph | H | H | b6 | H |
| 2-1-694 | O | N—Ph | t-bu | H | b6 | H |
| 2-1-695 | O | N—Ph | methyl | H | b6 | H |
| 2-1-696 | O | N—Ph | b1 | H | b6 | H |
| 2-1-697 | O | N—Ph | b2 | H | b6 | H |
| 2-1-698 | O | N—Ph | b3 | H | b6 | H |
| 2-1-699 | O | N—Ph | H | H | b7 | H |
| 2-1-700 | O | N—Ph | t-bu | H | b7 | H |
| 2-1-701 | O | N—Ph | methyl | H | b7 | H |
| 2-1-702 | O | N—Ph | b1 | H | b7 | H |
| 2-1-703 | O | N—Ph | b2 | H | b7 | H |
| 2-1-704 | O | N—Ph | b3 | H | b7 | H |
| 2-1-705 | O | N—Ph | H | H | b8 | H |
| 2-1-706 | O | N—Ph | t-bu | H | b8 | H |
| 2-1-707 | O | N—Ph | methyl | H | b8 | H |
| 2-1-708 | O | N—Ph | b1 | H | b8 | H |
| 2-1-709 | O | N—Ph | b2 | H | b8 | H |
| 2-1-710 | O | N—Ph | b3 | H | b8 | H |
| 2-1-711 | O | N—Ph | H | H | b9 | H |
| 2-1-712 | O | N—Ph | t-bu | H | b9 | H |
| 2-1-713 | O | N—Ph | methyl | H | b9 | H |
| 2-1-714 | O | N—Ph | b1 | H | b9 | H |
| 2-1-715 | O | N—Ph | b2 | H | b9 | H |
| 2-1-716 | O | N—Ph | b3 | H | b9 | H |
| 2-1-717 | O | N—Ph | H | H | b10 | H |
| 2-1-718 | O | N—Ph | t-bu | H | b10 | H |
| 2-1-719 | O | N—Ph | methyl | H | b10 | H |
| 2-1-720 | O | N—Ph | b1 | H | b10 | H |
| 2-1-721 | O | N—Ph | b2 | H | b10 | H |
| 2-1-722 | O | N—Ph | b3 | H | b10 | H |
| 2-1-723 | O | N—Ph | H | H | a1 | a1 |
| 2-1-724 | O | N—Ph | t-bu | H | a1 | a1 |
| 2-1-725 | O | N—Ph | methyl | H | a1 | a1 |
| 2-1-726 | O | N—Ph | b1 | H | a1 | a1 |
| 2-1-727 | O | N—Ph | b2 | H | a1 | a1 |
| 2-1-728 | O | N—Ph | b3 | H | a1 | a1 |
| 2-1-729 | O | N—Ph | H | H | a2 | a2 |
| 2-1-730 | O | N—Ph | t-bu | H | a2 | a2 |
| 2-1-731 | O | N—Ph | methyl | H | a2 | a2 |
| 2-1-732 | O | N—Ph | b1 | H | a2 | a2 |
| 2-1-733 | O | N—Ph | b2 | H | a2 | a2 |
| 2-1-734 | O | N—Ph | b3 | H | a2 | a2 |
| 2-1-735 | O | N—Ph | H | H | a3 | a3 |
| 2-1-736 | O | N—Ph | t-bu | H | a3 | a3 |
| 2-1-737 | O | N—Ph | methyl | H | a3 | a3 |
| 2-1-738 | O | N—Ph | b1 | H | a3 | a3 |
| 2-1-739 | O | N—Ph | b2 | H | a3 | a3 |
| 2-1-740 | O | N—Ph | b3 | H | a3 | a3 |
| 2-1-741 | O | N—Ph | H | H | b1 | b1 |
| 2-1-742 | O | N—Ph | t-bu | H | b1 | b1 |
| 2-1-743 | O | N—Ph | methyl | H | b1 | b1 |
| 2-1-744 | O | N—Ph | b1 | H | b1 | b1 |
| 2-1-745 | O | N—Ph | b2 | H | b1 | b1 |
| 2-1-746 | O | N—Ph | b3 | H | b1 | b1 |
| 2-1-747 | O | N—Ph | H | H | b2 | b2 |
| 2-1-748 | O | N—Ph | t-bu | H | b2 | b2 |
| 2-1-749 | O | N—Ph | methyl | H | b2 | b2 |
| 2-1-750 | O | N—Ph | b1 | H | b2 | b2 |
| 2-1-751 | O | N—Ph | b2 | H | b2 | b2 |
| 2-1-752 | O | N—Ph | b3 | H | b2 | b2 |
| 2-1-753 | O | N—Ph | H | H | b3 | b3 |
| 2-1-754 | O | N—Ph | t-bu | H | b3 | b3 |
| 2-1-755 | O | N—Ph | methyl | H | b3 | b3 |
| 2-1-756 | O | N—Ph | b1 | H | b3 | b3 |
| 2-1-757 | O | N—Ph | b2 | H | b3 | b3 |
| 2-1-758 | O | N—Ph | b3 | H | b3 | b3 |
| 2-1-759 | O | N—Ph | H | H | b5 | b5 |
| 2-1-760 | O | N—Ph | t-bu | H | b5 | b5 |
| 2-1-761 | O | N—Ph | methyl | H | b5 | b5 |
| 2-1-762 | O | N—Ph | b1 | H | b5 | b5 |
| 2-1-763 | O | N—Ph | b2 | H | b5 | b5 |
| 2-1-764 | O | N—Ph | b3 | H | b5 | b5 |
| 2-1-765 | O | N—Ph | H | H | b6 | b6 |
| 2-1-766 | O | N—Ph | t-bu | H | b6 | b6 |
| 2-1-767 | O | N—Ph | methyl | H | b6 | b6 |
| 2-1-768 | O | N—Ph | b1 | H | b6 | b6 |
| 2-1-769 | O | N—Ph | b2 | H | b6 | b6 |
| 2-1-770 | O | N—Ph | b3 | H | b6 | b6 |
| 2-1-771 | O | N—Ph | H | H | b7 | b7 |
| 2-1-772 | O | N—Ph | t-bu | H | b7 | b7 |
| 2-1-773 | O | N—Ph | methyl | H | b7 | b7 |
| 2-1-774 | O | N—Ph | b1 | H | b7 | b7 |
| 2-1-775 | O | N—Ph | b2 | H | b7 | b7 |
| 2-1-776 | O | N—Ph | b3 | H | b7 | b7 |
| 2-1-777 | S | N—Ph | H | H | H | H |
| 2-1-778 | S | N—Ph | t-bu | H | H | H |
| 2-1-779 | S | N—Ph | methyl | H | H | H |
| 2-1-780 | S | N—Ph | t-bu | t-bu | t-bu | t-bu |
| 2-1-781 | S | N—Ph | methyl | methyl | methyl | methyl |
| 2-1-782 | S | N—Ph | b1 | H | H | H |
| 2-1-783 | S | N—Ph | b2 | H | H | H |
| 2-1-784 | S | N—Ph | b3 | H | H | H |
| 2-1-785 | S | N—Ph | H | H | H | a1 |
| 2-1-786 | S | N—Ph | t-bu | H | H | a1 |
| 2-1-787 | S | N—Ph | methyl | H | H | a1 |
| 2-1-788 | S | N—Ph | b1 | H | H | a1 |
| 2-1-789 | S | N—Ph | b2 | H | H | a1 |
| 2-1-790 | S | N—Ph | b3 | H | H | a1 |
| 2-1-791 | S | N—Ph | H | H | H | a2 |
| 2-1-792 | S | N—Ph | t-bu | H | H | a2 |
| 2-1-793 | S | N—Ph | methyl | H | H | a2 |
| 2-1-794 | S | N—Ph | b1 | H | H | a2 |
| 2-1-795 | S | N—Ph | b2 | H | H | a2 |
| 2-1-796 | S | N—Ph | b3 | H | H | a2 |
| 2-1-797 | S | N—Ph | H | H | H | a3 |
| 2-1-798 | S | N—Ph | t-bu | H | H | a3 |
| 2-1-799 | S | N—Ph | methyl | H | H | a3 |
| 2-1-800 | S | N—Ph | b1 | H | H | a3 |
| 2-1-801 | S | N—Ph | b2 | H | H | a3 |
| 2-1-802 | S | N—Ph | b3 | H | H | a3 |
| 2-1-803 | S | N—Ph | H | H | H | a4 |
| 2-1-804 | S | N—Ph | t-bu | H | H | a4 |
| 2-1-805 | S | N—Ph | methyl | H | H | a4 |
| 2-1-806 | S | N—Ph | b1 | H | H | a4 |
| 2-1-807 | S | N—Ph | b2 | H | H | a4 |
| 2-1-808 | S | N—Ph | b3 | H | H | a4 |
| 2-1-809 | S | N—Ph | H | H | H | a5 |
| 2-1-810 | S | N—Ph | t-bu | H | H | a5 |
| 2-1-811 | S | N—Ph | methyl | H | H | a5 |
| 2-1-812 | S | N—Ph | b1 | H | H | a5 |
| 2-1-813 | S | N—Ph | b2 | H | H | a5 |
| 2-1-814 | S | N—Ph | b3 | H | H | a5 |
| 2-1-815 | S | N—Ph | H | H | H | a6 |
| 2-1-816 | S | N—Ph | t-bu | H | H | a6 |
| 2-1-817 | S | N—Ph | methyl | H | H | a6 |
| 2-1-818 | S | N—Ph | b1 | H | H | a6 |
| 2-1-819 | S | N—Ph | b2 | H | H | a6 |
| 2-1-820 | S | N—Ph | b3 | H | H | a6 |
| 2-1-821 | S | N—Ph | H | H | a1 | H |
| 2-1-822 | S | N—Ph | t-bu | H | a1 | H |
| 2-1-823 | S | N—Ph | methyl | H | a1 | H |
| 2-1-824 | S | N—Ph | b1 | H | a1 | H |
| 2-1-825 | S | N—Ph | b2 | H | a1 | H |
| 2-1-826 | S | N—Ph | b3 | H | a1 | H |
| 2-1-827 | S | N—Ph | H | H | a2 | H |
| 2-1-828 | S | N—Ph | t-bu | H | a2 | H |
| 2-1-829 | S | N—Ph | methyl | H | a2 | H |
| 2-1-830 | S | N—Ph | b1 | H | a2 | H |
| 2-1-831 | S | N—Ph | b2 | H | a2 | H |
| 2-1-832 | S | N—Ph | b3 | H | a2 | H |
| 2-1-833 | S | N—Ph | H | H | a3 | H |
| 2-1-834 | S | N—Ph | t-bu | H | a3 | H |
| 2-1-835 | S | N—Ph | methyl | H | a3 | H |
| 2-1-836 | S | N—Ph | b1 | H | a3 | H |
| 2-1-837 | S | N—Ph | b2 | H | a3 | H |
| 2-1-838 | S | N—Ph | b3 | H | a3 | H |
| 2-1-839 | S | N—Ph | H | H | a4 | H |
| 2-1-840 | S | N—Ph | t-bu | H | a4 | H |
| 2-1-841 | S | N—Ph | methyl | H | a4 | H |
| 2-1-842 | S | N—Ph | b1 | H | a4 | H |
| 2-1-843 | S | N—Ph | b2 | H | a4 | H |
| 2-1-844 | S | N—Ph | b3 | H | a4 | H |
| 2-1-845 | S | N—Ph | H | H | a5 | H |
| 2-1-846 | S | N—Ph | t-bu | H | a5 | H |
| 2-1-847 | S | N—Ph | methyl | H | a5 | H |
| 2-1-848 | S | N—Ph | b1 | H | a5 | H |
| 2-1-849 | S | N—Ph | b2 | H | a5 | H |
| 2-1-850 | S | N—Ph | b3 | H | a5 | H |
| 2-1-851 | S | N—Ph | H | H | a6 | H |
| 2-1-852 | S | N—Ph | t-bu | H | a6 | H |
| 2-1-853 | S | N—Ph | methyl | H | a6 | H |
| 2-1-854 | S | N—Ph | b1 | H | a6 | H |
| 2-1-855 | S | N—Ph | b2 | H | a6 | H |
| 2-1-856 | S | N—Ph | b3 | H | a6 | H |
| 2-1-857 | S | N—Ph | H | H | b1 | H |
| 2-1-858 | S | N—Ph | t-bu | H | b1 | H |
| 2-1-859 | S | N—Ph | methyl | H | b1 | H |
| 2-1-860 | S | N—Ph | b1 | H | b1 | H |
| 2-1-861 | S | N—Ph | b2 | H | b1 | H |
| 2-1-862 | S | N—Ph | b3 | H | b1 | H |
| 2-1-863 | S | N—Ph | H | H | b2 | H |
| 2-1-864 | S | N—Ph | t-bu | H | b2 | H |
| 2-1-865 | S | N—Ph | methyl | H | b2 | H |
| 2-1-866 | S | N—Ph | b1 | H | b2 | H |
| 2-1-867 | S | N—Ph | b2 | H | b2 | H |
| 2-1-868 | S | N—Ph | b3 | H | b2 | H |
| 2-1-869 | S | N—Ph | H | H | b3 | H |
| 2-1-870 | S | N—Ph | t-bu | H | b3 | H |
| 2-1-871 | S | N—Ph | methyl | H | b3 | H |
| 2-1-872 | S | N—Ph | b1 | H | b3 | H |
| 2-1-873 | S | N—Ph | b2 | H | b3 | H |
| 2-1-874 | S | N—Ph | b3 | H | b3 | H |
| 2-1-875 | S | N—Ph | H | H | b4 | H |
| 2-1-876 | S | N—Ph | t-bu | H | b4 | H |
| 2-1-877 | S | N—Ph | methyl | H | b4 | H |
| 2-1-878 | S | N—Ph | b1 | H | b4 | H |
| 2-1-879 | S | N—Ph | b2 | H | b4 | H |
| 2-1-880 | S | N—Ph | b3 | H | b4 | H |
| 2-1-881 | S | N—Ph | H | H | b5 | H |
| 2-1-882 | S | N—Ph | t-bu | H | b5 | H |
| 2-1-883 | S | N—Ph | methyl | H | b5 | H |
| 2-1-884 | S | N—Ph | b1 | H | b5 | H |
| 2-1-885 | S | N—Ph | b2 | H | b5 | H |
| 2-1-886 | S | N—Ph | b3 | H | b5 | H |
| 2-1-887 | S | N—Ph | H | H | b6 | H |
| 2-1-888 | S | N—Ph | t-bu | H | b6 | H |
| 2-1-889 | S | N—Ph | methyl | H | b6 | H |
| 2-1-890 | S | N—Ph | b1 | H | b6 | H |
| 2-1-891 | S | N—Ph | b2 | H | b6 | H |
| 2-1-892 | S | N—Ph | b3 | H | b6 | H |
| 2-1-893 | S | N—Ph | H | H | b7 | H |
| 2-1-894 | S | N—Ph | t-bu | H | b7 | H |
| 2-1-895 | S | N—Ph | methyl | H | b7 | H |
| 2-1-896 | S | N—Ph | b1 | H | b7 | H |
| 2-1-897 | S | N—Ph | b2 | H | b7 | H |
| 2-1-898 | S | N—Ph | b3 | H | b7 | H |
| 2-1-899 | S | N—Ph | H | H | b8 | H |
| 2-1-900 | S | N—Ph | t-bu | H | b8 | H |
| 2-1-901 | S | N—Ph | methyl | H | b8 | H |
| 2-1-902 | S | N—Ph | b1 | H | b8 | H |
| 2-1-903 | S | N—Ph | b2 | H | b8 | H |
| 2-1-904 | S | N—Ph | b3 | H | b8 | H |
| 2-1-905 | S | N—Ph | H | H | b9 | H |
| 2-1-906 | S | N—Ph | t-bu | H | b9 | H |
| 2-1-907 | S | N—Ph | methyl | H | b9 | H |
| 2-1-908 | S | N—Ph | b1 | H | b9 | H |
| 2-1-909 | S | N—Ph | b2 | H | b9 | H |
| 2-1-910 | S | N—Ph | b3 | H | b9 | H |
| 2-1-911 | S | N—Ph | H | H | b10 | H |
| 2-1-912 | S | N—Ph | t-bu | H | b10 | H |
| 2-1-913 | S | N—Ph | methyl | H | b10 | H |
| 2-1-914 | S | N—Ph | b1 | H | b10 | H |
| 2-1-915 | S | N—Ph | b2 | H | b10 | H |
| 2-1-916 | S | N—Ph | b3 | H | b10 | H |
| 2-1-917 | S | N—Ph | H | H | a1 | a1 |
| 2-1-918 | S | N—Ph | t-bu | H | a1 | a1 |
| 2-1-919 | S | N—Ph | methyl | H | a1 | a1 |
| 2-1-920 | S | N—Ph | b1 | H | a1 | a1 |
| 2-1-921 | S | N—Ph | b2 | H | a1 | a1 |
| 2-1-922 | S | N—Ph | b3 | H | a1 | a1 |
| 2-1-923 | S | N—Ph | H | H | a2 | a2 |
| 2-1-924 | S | N—Ph | t-bu | H | a2 | a2 |
| 2-1-925 | S | N—Ph | methyl | H | a2 | a2 |
| 2-1-926 | S | N—Ph | b1 | H | a2 | a2 |
| 2-1-927 | S | N—Ph | b2 | H | a2 | a2 |
| 2-1-928 | S | N—Ph | b3 | H | a2 | a2 |
| 2-1-929 | S | N—Ph | H | H | a3 | a3 |
| 2-1-930 | S | N—Ph | t-bu | H | a3 | a3 |
| 2-1-931 | S | N—Ph | methyl | H | a3 | a3 |
| 2-1-932 | S | N—Ph | b1 | H | a3 | a3 |
| 2-1-933 | S | N—Ph | b2 | H | a3 | a3 |
| 2-1-934 | S | N—Ph | b3 | H | a3 | a3 |
| 2-1-935 | S | N—Ph | H | H | b1 | b1 |
| 2-1-936 | S | N—Ph | t-bu | H | b1 | b1 |
| 2-1-937 | S | N—Ph | methyl | H | b1 | b1 |
| 2-1-938 | S | N—Ph | b1 | H | b1 | b1 |
| 2-1-939 | S | N—Ph | b2 | H | b1 | b1 |
| 2-1-940 | S | N—Ph | b3 | H | b1 | b1 |
| 2-1-941 | S | N—Ph | H | H | b2 | b2 |
| 2-1-942 | S | N—Ph | t-bu | H | b2 | b2 |
| 2-1-943 | S | N—Ph | methyl | H | b2 | b2 |
| 2-1-944 | S | N—Ph | b1 | H | b2 | b2 |
| 2-1-945 | S | N—Ph | b2 | H | b2 | b2 |
| 2-1-946 | S | N—Ph | b3 | H | b2 | b2 |
| 2-1-947 | S | N—Ph | H | H | b3 | b3 |
| 2-1-948 | S | N—Ph | t-bu | H | b3 | b3 |
| 2-1-949 | S | N—Ph | methyl | H | b3 | b3 |
| 2-1-950 | S | N—Ph | b1 | H | b3 | b3 |
| 2-1-951 | S | N—Ph | b2 | H | b3 | b3 |
| 2-1-952 | S | N—Ph | b3 | H | b3 | b3 |
| 2-1-953 | S | N—Ph | H | H | b5 | b5 |
| 2-1-954 | S | N—Ph | t-bu | H | b5 | b5 |
| 2-1-955 | S | N—Ph | methyl | H | b5 | b5 |
| 2-1-956 | S | N—Ph | b1 | H | b5 | b5 |
| 2-1-957 | S | N—Ph | b2 | H | b5 | b5 |
| 2-1-958 | S | N—Ph | b3 | H | b5 | b5 |
| 2-1-959 | S | N—Ph | H | H | b6 | b6 |
| 2-1-960 | S | N—Ph | t-bu | H | b6 | b6 |
| 2-1-961 | S | N—Ph | methyl | H | b6 | b6 |
| 2-1-962 | S | N—Ph | b1 | H | b6 | b6 |
| 2-1-963 | S | N—Ph | b2 | H | b6 | b6 |
| 2-1-964 | S | N—Ph | b3 | H | b6 | b6 |
| 2-1-965 | S | N—Ph | H | H | b7 | b7 |
| 2-1-966 | S | N—Ph | t-bu | H | b7 | b7 |
| 2-1-967 | S | N—Ph | methyl | H | b7 | b7 |
| 2-1-968 | S | N—Ph | b1 | H | b7 | b7 |
| 2-1-969 | S | N—Ph | b2 | H | b7 | b7 |
| 2-1-970 | S | N—Ph | b3 | H | b7 | b7 |

Group 2-2
The compounds are represented by the formula 2-2 below, wherein CY₂ and CY₃ are each a benzene group, X₁ is B, and Y1, Y2, R1a, R1c, (R2)a2, (R3)a3 are each shown in the table 2 below, 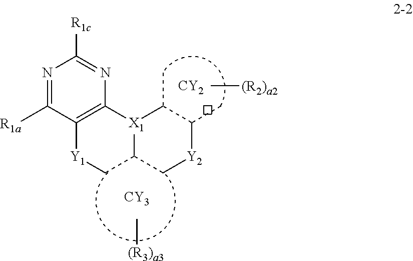
TABLE 2

| Compound No. | Y1 | Y2 | R1a | R1c | (R2)a2 | (R3)a3 |
|---|---|---|---|---|---|---|
| 2-2-001 | N—Ph | N—Ph | H | H | H | H |
| 2-2-002 | N—Ph | N—Ph | t-bu | t-bu | H | H |
| 2-2-003 | N—Ph | N—Ph | methyl | methyl | H | H |
| 2-2-004 | N—Ph | N—Ph | t-bu | t-bu | t-bu | t-bu |
| 2-2-005 | N—Ph | N—Ph | methyl | methyl | methyl | methyl |
| 2-2-006 | N—Ph | N—Ph | b1 | b1 | H | H |
| 2-2-007 | N—Ph | N—Ph | b2 | b2 | H | H |
| 2-2-008 | N—Ph | N—Ph | b3 | b3 | H | H |
| 2-2-009 | N—Ph | N—Ph | H | H | H | a1 |
| 2-2-010 | N—Ph | N—Ph | t-bu | t-bu | H | a1 |
| 2-2-011 | N—Ph | N—Ph | methyl | methyl | H | a1 |
| 2-2-012 | N—Ph | N—Ph | b1 | b1 | H | a1 |
| 2-2-013 | N—Ph | N—Ph | b2 | b2 | H | a1 |
| 2-2-014 | N—Ph | N—Ph | b3 | b3 | H | a1 |
| 2-2-015 | N—Ph | N—Ph | H | H | H | a2 |
| 2-2-016 | N—Ph | N—Ph | t-bu | t-bu | H | a2 |
| 2-2-017 | N—Ph | N—Ph | methyl | methyl | H | a2 |
| 2-2-018 | N—Ph | N—Ph | b1 | b1 | H | a2 |
| 2-2-019 | N—Ph | N—Ph | b2 | b2 | H | a2 |
| 2-2-020 | N—Ph | N—Ph | b3 | b3 | H | a2 |
| 2-2-021 | N—Ph | N—Ph | H | H | H | a3 |
| 2-2-022 | N—Ph | N—Ph | t-bu | t-bu | H | a3 |
| 2-2-023 | N—Ph | N—Ph | methyl | methyl | H | a3 |
| 2-2-024 | N—Ph | N—Ph | b1 | b1 | H | a3 |
| 2-2-025 | N—Ph | N—Ph | b2 | b2 | H | a3 |
| 2-2-026 | N—Ph | N—Ph | b3 | b3 | H | a3 |
| 2-2-027 | N—Ph | N—Ph | H | H | H | a4 |
| 2-2-028 | N—Ph | N—Ph | t-bu | t-bu | H | a4 |
| 2-2-029 | N—Ph | N—Ph | methyl | methyl | H | a4 |
| 2-2-030 | N—Ph | N—Ph | b1 | b1 | H | a4 |
| 2-2-031 | N—Ph | N—Ph | b2 | b2 | H | a4 |
| 2-2-032 | N—Ph | N—Ph | b3 | b3 | H | a4 |
| 2-2-033 | N—Ph | N—Ph | H | H | H | a5 |
| 2-2-034 | N—Ph | N—Ph | t-bu | t-bu | H | a5 |
| 2-2-035 | N—Ph | N—Ph | methyl | methyl | H | a5 |
| 2-2-036 | N—Ph | N—Ph | b1 | b1 | H | a5 |
| 2-2-037 | N—Ph | N—Ph | b2 | b2 | H | a5 |
| 2-2-038 | N—Ph | N—Ph | b3 | b3 | H | a5 |
| 2-2-039 | N—Ph | N—Ph | H | H | H | a6 |
| 2-2-040 | N—Ph | N—Ph | t-bu | t-bu | H | a6 |
| 2-2-041 | N—Ph | N—Ph | methyl | methyl | H | a6 |
| 2-2-042 | N—Ph | N—Ph | b1 | b1 | H | a6 |
| 2-2-043 | N—Ph | N—Ph | b2 | b2 | H | a6 |
| 2-2-044 | N—Ph | N—Ph | b3 | b3 | H | a6 |
| 2-2-045 | N—Ph | N—Ph | H | H | a1 | H |
| 2-2-046 | N—Ph | N—Ph | t-bu | t-bu | a1 | H |
| 2-2-047 | N—Ph | N—Ph | methyl | methyl | a1 | H |
| 2-2-048 | N—Ph | N—Ph | b1 | b1 | a1 | H |
| 2-2-049 | N—Ph | N—Ph | b2 | b2 | a1 | H |
| 2-2-050 | N—Ph | N—Ph | b3 | b3 | a1 | H |
| 2-2-051 | N—Ph | N—Ph | H | H | a2 | H |
| 2-2-052 | N—Ph | N—Ph | t-bu | t-bu | a2 | H |
| 2-2-053 | N—Ph | N—Ph | methyl | methyl | a2 | H |
| 2-2-054 | N—Ph | N—Ph | b1 | b1 | a2 | H |
| 2-2-055 | N—Ph | N—Ph | b2 | b2 | a2 | H |
| 2-2-056 | N—Ph | N—Ph | b3 | b3 | a2 | H |
| 2-2-057 | N—Ph | N—Ph | H | H | a3 | H |
| 2-2-058 | N—Ph | N—Ph | t-bu | t-bu | a3 | H |
| 2-2-059 | N—Ph | N—Ph | methyl | methyl | a3 | H |
| 2-2-060 | N—Ph | N—Ph | b1 | b1 | a3 | H |
| 2-2-061 | N—Ph | N—Ph | b2 | b2 | a3 | H |
| 2-2-062 | N—Ph | N—Ph | b3 | b3 | a3 | H |
| 2-2-063 | N—Ph | N—Ph | H | H | a4 | H |
| 2-2-064 | N—Ph | N—Ph | t-bu | t-bu | a4 | H |
| 2-2-065 | N—Ph | N—Ph | methyl | methyl | a4 | H |
| 2-2-066 | N—Ph | N—Ph | b1 | b1 | a4 | H |
| 2-2-067 | N—Ph | N—Ph | b2 | b2 | a4 | H |
| 2-2-068 | N—Ph | N—Ph | b3 | b3 | a4 | H |
| 2-2-069 | N—Ph | N—Ph | H | H | a5 | H |
| 2-2-070 | N—Ph | N—Ph | t-bu | t-bu | a5 | H |
| 2-2-071 | N—Ph | N—Ph | methyl | methyl | a5 | H |
| 2-2-072 | N—Ph | N—Ph | b1 | b1 | a5 | H |
| 2-2-073 | N—Ph | N—Ph | b2 | b2 | a5 | H |
| 2-2-074 | N—Ph | N—Ph | b3 | b3 | a5 | H |
| 2-2-075 | N—Ph | N—Ph | H | H | a6 | H |
| 2-2-076 | N—Ph | N—Ph | t-bu | t-bu | a6 | H |
| 2-2-077 | N—Ph | N—Ph | methyl | methyl | a6 | H |
| 2-2-078 | N—Ph | N—Ph | b1 | b1 | a6 | H |
| 2-2-079 | N—Ph | N—Ph | b2 | b2 | a6 | H |
| 2-2-080 | N—Ph | N—Ph | b3 | b3 | a6 | H |
| 2-2-081 | N—Ph | N—Ph | H | H | b1 | H |
| 2-2-082 | N—Ph | N—Ph | t-bu | t-bu | b1 | H |
| 2-2-083 | N—Ph | N—Ph | methyl | methyl | b1 | H |
| 2-2-084 | N—Ph | N—Ph | b1 | b1 | b1 | H |
| 2-2-085 | N—Ph | N—Ph | b2 | b2 | b1 | H |
| 2-2-086 | N—Ph | N—Ph | b3 | b3 | b1 | H |
| 2-2-087 | N—Ph | N—Ph | H | H | b2 | H |
| 2-2-088 | N—Ph | N—Ph | t-bu | t-bu | b2 | H |
| 2-2-089 | N—Ph | N—Ph | methyl | methyl | b2 | H |
| 2-2-090 | N—Ph | N—Ph | b1 | b1 | b2 | H |
| 2-2-091 | N—Ph | N—Ph | b2 | b2 | b2 | H |
| 2-2-092 | N—Ph | N—Ph | b3 | b3 | b2 | H |
| 2-2-093 | N—Ph | N—Ph | H | H | b3 | H |
| 2-2-094 | N—Ph | N—Ph | t-bu | t-bu | b3 | H |
| 2-2-095 | N—Ph | N—Ph | methyl | methyl | b3 | H |
| 2-2-096 | N—Ph | N—Ph | b1 | b1 | b3 | H |
| 2-2-097 | N—Ph | N—Ph | b2 | b2 | b3 | H |
| 2-2-098 | N—Ph | N—Ph | b3 | b3 | b3 | H |
| 2-2-099 | N—Ph | N—Ph | H | H | b4 | H |
| 2-2-100 | N—Ph | N—Ph | t-bu | t-bu | b4 | H |
| 2-2-101 | N—Ph | N—Ph | methyl | methyl | b4 | H |
| 2-2-102 | N—Ph | N—Ph | b1 | b1 | b4 | H |
| 2-2-103 | N—Ph | N—Ph | b2 | b2 | b4 | H |
| 2-2-104 | N—Ph | N—Ph | b3 | b3 | b4 | H |
| 2-2-105 | N—Ph | N—Ph | H | H | b5 | H |
| 2-2-106 | N—Ph | N—Ph | t-bu | t-bu | b5 | H |
| 2-2-107 | N—Ph | N—Ph | methyl | methyl | b5 | H |
| 2-2-108 | N—Ph | N—Ph | b1 | b1 | b5 | H |
| 2-2-109 | N—Ph | N—Ph | b2 | b2 | b5 | H |
| 2-2-110 | N—Ph | N—Ph | b3 | b3 | b5 | H |
| 2-2-111 | N—Ph | N—Ph | H | H | b6 | H |
| 2-2-112 | N—Ph | N—Ph | t-bu | t-bu | b6 | H |
| 2-2-113 | N—Ph | N—Ph | methyl | methyl | b6 | H |
| 2-2-114 | N—Ph | N—Ph | b1 | b1 | b6 | H |
| 2-2-115 | N—Ph | N—Ph | b2 | b2 | b6 | H |
| 2-2-116 | N—Ph | N—Ph | b3 | b3 | b6 | H |
| 2-2-117 | N—Ph | N—Ph | H | H | b7 | H |
| 2-2-118 | N—Ph | N—Ph | t-bu | t-bu | b7 | H |
| 2-2-119 | N—Ph | N—Ph | methyl | methyl | b7 | H |
| 2-2-120 | N—Ph | N—Ph | b1 | b1 | b7 | H |
| 2-2-121 | N—Ph | N—Ph | b2 | b2 | b7 | H |
| 2-2-122 | N—Ph | N—Ph | b3 | b3 | b7 | H |
| 2-2-123 | N—Ph | N—Ph | H | H | b8 | H |
| 2-2-124 | N—Ph | N—Ph | t-bu | t-bu | b8 | H |
| 2-2-125 | N—Ph | N—Ph | methyl | methyl | b8 | H |
| 2-2-126 | N—Ph | N—Ph | b1 | b1 | b8 | H |
| 2-2-127 | N—Ph | N—Ph | b2 | b2 | b8 | H |
| 2-2-128 | N—Ph | N—Ph | b3 | b3 | b8 | H |
| 2-2-129 | N—Ph | N—Ph | H | H | b9 | H |
| 2-2-130 | N—Ph | N—Ph | t-bu | t-bu | b9 | H |
| 2-2-131 | N—Ph | N—Ph | methyl | methyl | b9 | H |
| 2-2-132 | N—Ph | N—Ph | b1 | b1 | b9 | H |
| 2-2-133 | N—Ph | N—Ph | b2 | b2 | b9 | H |
| 2-2-134 | N—Ph | N—Ph | b3 | b3 | b9 | H |
| 2-2-135 | N—Ph | N—Ph | H | H | b10 | H |
| 2-2-136 | N—Ph | N—Ph | t-bu | t-bu | b10 | H |
| 2-2-137 | N—Ph | N—Ph | methyl | methyl | b10 | H |
| 2-2-138 | N—Ph | N—Ph | b1 | b1 | b10 | H |
| 2-2-139 | N—Ph | N—Ph | b2 | b2 | b10 | H |
| 2-2-140 | N—Ph | N—Ph | b3 | b3 | b10 | H |
| 2-2-141 | N—Ph | N—Ph | H | H | a1 | a1 |
| 2-2-142 | N—Ph | N—Ph | t-bu | t-bu | a1 | a1 |
| 2-2-143 | N—Ph | N—Ph | methyl | methyl | a1 | a1 |
| 2-2-144 | N—Ph | N—Ph | b1 | b1 | a1 | a1 |
| 2-2-145 | N—Ph | N—Ph | b2 | b2 | a1 | a1 |
| 2-2-146 | N—Ph | N—Ph | b3 | b3 | a1 | a1 |
| 2-2-147 | N—Ph | N—Ph | H | H | a2 | a2 |
| 2-2-148 | N—Ph | N—Ph | t-bu | t-bu | a2 | a2 |
| 2-2-149 | N—Ph | N—Ph | methyl | methyl | a2 | a2 |
| 2-2-150 | N—Ph | N—Ph | b1 | b1 | a2 | a2 |
| 2-2-151 | N—Ph | N—Ph | b2 | b2 | a2 | a2 |
| 2-2-152 | N—Ph | N—Ph | b3 | b3 | a2 | a2 |
| 2-2-153 | N—Ph | N—Ph | H | H | a3 | a3 |
| 2-2-154 | N—Ph | N—Ph | t-bu | t-bu | a3 | a3 |
| 2-2-155 | N—Ph | N—Ph | methyl | methyl | a3 | a3 |
| 2-2-156 | N—Ph | N—Ph | b1 | b1 | a3 | a3 |
| 2-2-157 | N—Ph | N—Ph | b2 | b2 | a3 | a3 |
| 2-2-158 | N—Ph | N—Ph | b3 | b3 | a3 | a3 |
| 2-2-159 | N—Ph | N—Ph | H | H | b1 | b1 |
| 2-2-160 | N—Ph | N—Ph | t-bu | t-bu | b1 | b1 |
| 2-2-161 | N—Ph | N—Ph | methyl | methyl | b1 | b1 |
| 2-2-162 | N—Ph | N—Ph | b1 | b1 | b1 | b1 |
| 2-2-163 | N—Ph | N—Ph | b2 | b2 | b1 | b1 |
| 2-2-164 | N—Ph | N—Ph | b3 | b3 | b1 | b1 |
| 2-2-165 | N—Ph | N—Ph | H | H | b2 | b2 |
| 2-2-166 | N—Ph | N—Ph | t-bu | t-bu | b2 | b2 |
| 2-2-167 | N—Ph | N—Ph | methyl | methyl | b2 | b2 |
| 2-2-168 | N—Ph | N—Ph | b1 | b1 | b2 | b2 |
| 2-2-169 | N—Ph | N—Ph | b2 | b2 | b2 | b2 |
| 2-2-170 | N—Ph | N—Ph | b3 | b3 | b2 | b2 |
| 2-2-171 | N—Ph | N—Ph | H | H | b3 | b3 |
| 2-2-172 | N—Ph | N—Ph | t-bu | t-bu | b3 | b3 |
| 2-2-173 | N—Ph | N—Ph | methyl | methyl | b3 | b3 |
| 2-2-174 | N—Ph | N—Ph | b1 | b1 | b3 | b3 |
| 2-2-175 | N—Ph | N—Ph | b2 | b2 | b3 | b3 |
| 2-2-176 | N—Ph | N—Ph | b3 | b3 | b3 | b3 |
| 2-2-177 | N—Ph | N—Ph | H | H | b5 | b5 |
| 2-2-178 | N—Ph | N—Ph | t-bu | t-bu | b5 | b5 |
| 2-2-179 | N—Ph | N—Ph | methyl | methyl | b5 | b5 |
| 2-2-180 | N—Ph | N—Ph | b1 | b1 | b5 | b5 |
| 2-2-181 | N—Ph | N—Ph | b2 | b2 | b5 | b5 |
| 2-2-182 | N—Ph | N—Ph | b3 | b3 | b5 | b5 |
| 2-2-183 | N—Ph | N—Ph | H | H | b6 | b6 |
| 2-2-184 | N—Ph | N—Ph | t-bu | t-bu | b6 | b6 |
| 2-2-185 | N—Ph | N—Ph | methyl | methyl | b6 | b6 |
| 2-2-186 | N—Ph | N—Ph | b1 | b1 | b6 | b6 |
| 2-2-187 | N—Ph | N—Ph | b2 | b2 | b6 | b6 |
| 2-2-188 | N—Ph | N—Ph | b3 | b3 | b6 | b6 |
| 2-2-189 | N—Ph | N—Ph | H | H | b7 | b7 |
| 2-2-190 | N—Ph | N—Ph | t-bu | t-bu | b7 | b7 |
| 2-2-191 | N—Ph | N—Ph | methyl | methyl | b7 | b7 |
| 2-2-192 | N—Ph | N—Ph | b1 | b1 | b7 | b7 |
| 2-2-193 | N—Ph | N—Ph | b2 | b2 | b7 | b7 |
| 2-2-194 | N—Ph | N—Ph | b3 | b3 | b7 | b7 |
| 2-2-195 | N-tolyl | N-tolyl | H | H | H | H |
| 2-2-196 | N-tolyl | N-tolyl | t-bu | t-bu | H | H |
| 2-2-197 | N-tolyl | N-tolyl | methyl | methyl | H | H |
| 2-2-198 | N-tolyl | N-tolyl | t-bu | t-bu | t-bu | t-bu |
| 2-2-199 | N-tolyl | N-tolyl | methyl | methyl | methyl | methyl |
| 2-2-200 | N-tolyl | N-tolyl | b1 | b1 | H | H |
| 2-2-201 | N-tolyl | N-tolyl | b2 | b2 | H | H |
| 2-2-202 | N-tolyl | N-tolyl | b3 | b3 | H | H |
| 2-2-203 | N-tolyl | N-tolyl | H | H | H | a1 |
| 2-2-204 | N-tolyl | N-tolyl | t-bu | t-bu | H | a1 |
| 2-2-205 | N-tolyl | N-tolyl | methyl | methyl | H | a1 |
| 2-2-206 | N-tolyl | N-tolyl | b1 | b1 | H | a1 |
| 2-2-207 | N-tolyl | N-tolyl | b2 | b2 | H | a1 |
| 2-2-208 | N-tolyl | N-tolyl | b3 | b3 | H | a1 |
| 2-2-209 | N-tolyl | N-tolyl | H | H | H | a2 |
| 2-2-210 | N-tolyl | N-tolyl | t-bu | t-bu | H | a2 |
| 2-2-211 | N-tolyl | N-tolyl | methyl | methyl | H | a2 |
| 2-2-212 | N-tolyl | N-tolyl | b1 | b1 | H | a2 |
| 2-2-213 | N-tolyl | N-tolyl | b2 | b2 | H | a2 |
| 2-2-214 | N-tolyl | N-tolyl | b3 | b3 | H | a2 |
| 2-2-215 | N-tolyl | N-tolyl | H | H | H | a3 |
| 2-2-216 | N-tolyl | N-tolyl | t-bu | t-bu | H | a3 |
| 2-2-217 | N-tolyl | N-tolyl | methyl | methyl | H | a3 |
| 2-2-218 | N-tolyl | N-tolyl | b1 | b1 | H | a3 |
| 2-2-219 | N-tolyl | N-tolyl | b2 | b2 | H | a3 |
| 2-2-220 | N-tolyl | N-tolyl | b3 | b3 | H | a3 |
| 2-2-221 | N-tolyl | N-tolyl | H | H | H | a4 |
| 2-2-222 | N-tolyl | N-tolyl | t-bu | t-bu | H | a4 |
| 2-2-223 | N-tolyl | N-tolyl | methyl | methyl | H | a4 |
| 2-2-224 | N-tolyl | N-tolyl | b1 | b1 | H | a4 |
| 2-2-225 | N-tolyl | N-tolyl | b2 | b2 | H | a4 |
| 2-2-226 | N-tolyl | N-tolyl | b3 | b3 | H | a4 |
| 2-2-227 | N-tolyl | N-tolyl | H | H | H | a5 |
| 2-2-228 | N-tolyl | N-tolyl | t-bu | t-bu | H | a5 |
| 2-2-229 | N-tolyl | N-tolyl | methyl | methyl | H | a5 |
| 2-2-230 | N-tolyl | N-tolyl | b1 | b1 | H | a5 |
| 2-2-231 | N-tolyl | N-tolyl | b2 | b2 | H | a5 |
| 2-2-232 | N-tolyl | N-tolyl | b3 | b3 | H | a5 |
| 2-2-233 | N-tolyl | N-tolyl | H | H | H | a6 |
| 2-2-234 | N-tolyl | N-tolyl | t-bu | t-bu | H | a6 |
| 2-2-235 | N-tolyl | N-tolyl | methyl | methyl | H | a6 |
| 2-2-236 | N-tolyl | N-tolyl | b1 | b1 | H | a6 |
| 2-2-237 | N-tolyl | N-tolyl | b2 | b2 | H | a6 |
| 2-2-238 | N-tolyl | N-tolyl | b3 | b3 | H | a6 |
| 2-2-239 | N-tolyl | N-tolyl | H | H | a1 | H |
| 2-2-240 | N-tolyl | N-tolyl | t-bu | t-bu | a1 | H |
| 2-2-241 | N-tolyl | N-tolyl | methyl | methyl | a1 | H |
| 2-2-242 | N-tolyl | N-tolyl | b1 | b1 | a1 | H |
| 2-2-243 | N-tolyl | N-tolyl | b2 | b2 | a1 | H |
| 2-2-244 | N-tolyl | N-tolyl | b3 | b3 | a1 | H |
| 2-2-245 | N-tolyl | N-tolyl | H | H | a2 | H |
| 2-2-246 | N-tolyl | N-tolyl | t-bu | t-bu | a2 | H |
| 2-2-247 | N-tolyl | N-tolyl | methyl | methyl | a2 | H |
| 2-2-248 | N-tolyl | N-tolyl | b1 | b1 | a2 | H |
| 2-2-249 | N-tolyl | N-tolyl | b2 | b2 | a2 | H |
| 2-2-250 | N-tolyl | N-tolyl | b3 | b3 | a2 | H |
| 2-2-251 | N-tolyl | N-tolyl | H | H | a3 | H |
| 2-2-252 | N-tolyl | N-tolyl | t-bu | t-bu | a3 | H |
| 2-2-253 | N-tolyl | N-tolyl | methyl | methyl | a3 | H |
| 2-2-254 | N-tolyl | N-tolyl | b1 | b1 | a3 | H |
| 2-2-255 | N-tolyl | N-tolyl | b2 | b2 | a3 | H |
| 2-2-256 | N-tolyl | N-tolyl | b3 | b3 | a3 | H |
| 2-2-257 | N-tolyl | N-tolyl | H | H | a4 | H |
| 2-2-258 | N-tolyl | N-tolyl | t-bu | t-bu | a4 | H |
| 2-2-259 | N-tolyl | N-tolyl | methyl | methyl | a4 | H |
| 2-2-260 | N-tolyl | N-tolyl | b1 | b1 | a4 | H |
| 2-2-261 | N-tolyl | N-tolyl | b2 | b2 | a4 | H |
| 2-2-262 | N-tolyl | N-tolyl | b3 | b3 | a4 | H |
| 2-2-263 | N-tolyl | N-tolyl | H | H | a5 | H |
| 2-2-264 | N-tolyl | N-tolyl | t-bu | t-bu | a5 | H |
| 2-2-265 | N-tolyl | N-tolyl | methyl | methyl | a5 | H |
| 2-2-266 | N-tolyl | N-tolyl | b1 | b1 | a5 | H |
| 2-2-267 | N-tolyl | N-tolyl | b2 | b2 | a5 | H |
| 2-2-268 | N-tolyl | N-tolyl | b3 | b3 | a5 | H |
| 2-2-269 | N-tolyl | N-tolyl | H | H | a6 | H |
| 2-2-270 | N-tolyl | N-tolyl | t-bu | t-bu | a6 | H |
| 2-2-271 | N-tolyl | N-tolyl | methyl | methyl | a6 | H |
| 2-2-272 | N-tolyl | N-tolyl | b1 | b1 | a6 | H |
| 2-2-273 | N-tolyl | N-tolyl | b2 | b2 | a6 | H |
| 2-2-274 | N-tolyl | N-tolyl | b3 | b3 | a6 | H |
| 2-2-275 | N-tolyl | N-tolyl | H | H | b1 | H |
| 2-2-276 | N-tolyl | N-tolyl | t-bu | t-bu | b1 | H |
| 2-2-277 | N-tolyl | N-tolyl | methyl | methyl | b1 | H |
| 2-2-278 | N-tolyl | N-tolyl | b1 | b1 | b1 | H |
| 2-2-279 | N-tolyl | N-tolyl | b2 | b2 | b1 | H |
| 2-2-280 | N-tolyl | N-tolyl | b3 | b3 | b1 | H |
| 2-2-281 | N-tolyl | N-tolyl | H | H | b2 | H |
| 2-2-282 | N-tolyl | N-tolyl | t-bu | t-bu | b2 | H |
| 2-2-283 | N-tolyl | N-tolyl | methyl | methyl | b2 | H |
| 2-2-284 | N-tolyl | N-tolyl | b1 | b1 | b2 | H |
| 2-2-285 | N-tolyl | N-tolyl | b2 | b2 | b2 | H |
| 2-2-286 | N-tolyl | N-tolyl | b3 | b3 | b2 | H |
| 2-2-287 | N-tolyl | N-tolyl | H | H | b3 | H |
| 2-2-288 | N-tolyl | N-tolyl | t-bu | t-bu | b3 | H |
| 2-2-289 | N-tolyl | N-tolyl | methyl | methyl | b3 | H |
| 2-2-290 | N-tolyl | N-tolyl | b1 | b1 | b3 | H |
| 2-2-291 | N-tolyl | N-tolyl | b2 | b2 | b3 | H |
| 2-2-292 | N-tolyl | N-tolyl | b3 | b3 | b3 | H |
| 2-2-293 | N-tolyl | N-tolyl | H | H | b4 | H |
| 2-2-294 | N-tolyl | N-tolyl | t-bu | t-bu | b4 | H |
| 2-2-295 | N-tolyl | N-tolyl | methyl | methyl | b4 | H |
| 2-2-296 | N-tolyl | N-tolyl | b1 | b1 | b4 | H |
| 2-2-297 | N-tolyl | N-tolyl | b2 | b2 | b4 | H |
| 2-2-298 | N-tolyl | N-tolyl | b3 | b3 | b4 | H |
| 2-2-299 | N-tolyl | N-tolyl | H | H | b5 | H |
| 2-2-300 | N-tolyl | N-tolyl | t-bu | t-bu | b5 | H |
| 2-2-301 | N-tolyl | N-tolyl | methyl | methyl | b5 | H |
| 2-2-302 | N-tolyl | N-tolyl | b1 | b1 | b5 | H |
| 2-2-303 | N-tolyl | N-tolyl | b2 | b2 | b5 | H |
| 2-2-304 | N-tolyl | N-tolyl | b3 | b3 | b5 | H |
| 2-2-305 | N-tolyl | N-tolyl | H | H | b6 | H |
| 2-2-306 | N-tolyl | N-tolyl | t-bu | t-bu | b6 | H |
| 2-2-307 | N-tolyl | N-tolyl | methyl | methyl | b6 | H |
| 2-2-308 | N-tolyl | N-tolyl | b1 | b1 | b6 | H |
| 2-2-309 | N-tolyl | N-tolyl | b2 | b2 | b6 | H |
| 2-2-310 | N-tolyl | N-tolyl | b3 | b3 | b6 | H |
| 2-2-311 | N-tolyl | N-tolyl | H | H | b7 | H |
| 2-2-312 | N-tolyl | N-tolyl | t-bu | t-bu | b7 | H |
| 2-2-313 | N-tolyl | N-tolyl | methyl | methyl | b7 | H |
| 2-2-314 | N-tolyl | N-tolyl | b1 | b1 | b7 | H |
| 2-2-315 | N-tolyl | N-tolyl | b2 | b2 | b7 | H |
| 2-2-316 | N-tolyl | N-tolyl | b3 | b3 | b7 | H |
| 2-2-317 | N-tolyl | N-tolyl | H | H | b8 | H |
| 2-2-318 | N-tolyl | N-tolyl | t-bu | t-bu | b8 | H |
| 2-2-319 | N-tolyl | N-tolyl | methyl | methyl | b8 | H |
| 2-2-320 | N-tolyl | N-tolyl | b1 | b1 | b8 | H |
| 2-2-321 | N-tolyl | N-tolyl | b2 | b2 | b8 | H |
| 2-2-322 | N-tolyl | N-tolyl | b3 | b3 | b8 | H |
| 2-2-323 | N-tolyl | N-tolyl | H | H | b9 | H |
| 2-2-324 | N-tolyl | N-tolyl | t-bu | t-bu | b9 | H |
| 2-2-325 | N-tolyl | N-tolyl | methyl | methyl | b9 | H |
| 2-2-326 | N-tolyl | N-tolyl | b1 | b1 | b9 | H |
| 2-2-327 | N-tolyl | N-tolyl | b2 | b2 | b9 | H |
| 2-2-328 | N-tolyl | N-tolyl | b3 | b3 | b9 | H |
| 2-2-329 | N-tolyl | N-tolyl | H | H | b10 | H |
| 2-2-330 | N-tolyl | N-tolyl | t-bu | t-bu | b10 | H |
| 2-2-331 | N-tolyl | N-tolyl | methyl | methyl | b10 | H |
| 2-2-332 | N-tolyl | N-tolyl | b1 | b1 | b10 | H |
| 2-2-333 | N-tolyl | N-tolyl | b2 | b2 | b10 | H |
| 2-2-334 | N-tolyl | N-tolyl | b3 | b3 | b10 | H |
| 2-2-335 | N-tolyl | N-tolyl | H | H | a1 | a1 |
| 2-2-336 | N-tolyl | N-tolyl | t-bu | t-bu | a1 | a1 |
| 2-2-337 | N-tolyl | N-tolyl | methyl | methyl | a1 | a1 |
| 2-2-338 | N-tolyl | N-tolyl | b1 | b1 | a1 | a1 |
| 2-2-339 | N-tolyl | N-tolyl | b2 | b2 | a1 | a1 |
| 2-2-340 | N-tolyl | N-tolyl | b3 | b3 | a1 | a1 |
| 2-2-341 | N-tolyl | N-tolyl | H | H | a2 | a2 |
| 2-2-342 | N-tolyl | N-tolyl | t-bu | t-bu | a2 | a2 |
| 2-2-343 | N-tolyl | N-tolyl | methyl | methyl | a2 | a2 |
| 2-2-344 | N-tolyl | N-tolyl | b1 | b1 | a2 | a2 |
| 2-2-345 | N-tolyl | N-tolyl | b2 | b2 | a2 | a2 |
| 2-2-346 | N-tolyl | N-tolyl | b3 | b3 | a2 | a2 |
| 2-2-347 | N-tolyl | N-tolyl | H | H | a3 | a3 |
| 2-2-348 | N-tolyl | N-tolyl | t-bu | t-bu | a3 | a3 |
| 2-2-349 | N-tolyl | N-tolyl | methyl | methyl | a3 | a3 |
| 2-2-350 | N-tolyl | N-tolyl | b1 | b1 | a3 | a3 |
| 2-2-351 | N-tolyl | N-tolyl | b2 | b2 | a3 | a3 |
| 2-2-352 | N-tolyl | N-tolyl | b3 | b3 | a3 | a3 |
| 2-2-353 | N-tolyl | N-tolyl | H | H | b1 | b1 |
| 2-2-354 | N-tolyl | N-tolyl | t-bu | t-bu | b1 | b1 |
| 2-2-355 | N-tolyl | N-tolyl | methyl | methyl | b1 | b1 |
| 2-2-356 | N-tolyl | N-tolyl | b1 | b1 | b1 | b1 |
| 2-2-357 | N-tolyl | N-tolyl | b2 | b2 | b1 | b1 |
| 2-2-358 | N-tolyl | N-tolyl | b3 | b3 | b1 | b1 |
| 2-2-359 | N-tolyl | N-tolyl | H | H | b2 | b2 |
| 2-2-360 | N-tolyl | N-tolyl | t-bu | t-bu | b2 | b2 |
| 2-2-361 | N-tolyl | N-tolyl | methyl | methyl | b2 | b2 |
| 2-2-362 | N-tolyl | N-tolyl | b1 | b1 | b2 | b2 |
| 2-2-363 | N-tolyl | N-tolyl | b2 | b2 | b2 | b2 |
| 2-2-364 | N-tolyl | N-tolyl | b3 | b3 | b2 | b2 |
| 2-2-365 | N-tolyl | N-tolyl | H | H | b3 | b3 |
| 2-2-366 | N-tolyl | N-tolyl | t-bu | t-bu | b3 | b3 |
| 2-2-367 | N-tolyl | N-tolyl | methyl | methyl | b3 | b3 |
| 2-2-368 | N-tolyl | N-tolyl | b1 | b1 | b3 | b3 |
| 2-2-369 | N-tolyl | N-tolyl | b2 | b2 | b3 | b3 |
| 2-2-370 | N-tolyl | N-tolyl | b3 | b3 | b3 | b3 |
| 2-2-371 | N-tolyl | N-tolyl | H | H | b5 | b5 |
| 2-2-372 | N-tolyl | N-tolyl | t-bu | t-bu | b5 | b5 |
| 2-2-373 | N-tolyl | N-tolyl | methyl | methyl | b5 | b5 |
| 2-2-374 | N-tolyl | N-tolyl | b1 | b1 | b5 | b5 |
| 2-2-375 | N-tolyl | N-tolyl | b2 | b2 | b5 | b5 |
| 2-2-376 | N-tolyl | N-tolyl | b3 | b3 | b5 | b5 |
| 2-2-377 | N-tolyl | N-tolyl | H | H | b6 | b6 |
| 2-2-378 | N-tolyl | N-tolyl | t-bu | t-bu | b6 | b6 |
| 2-2-379 | N-tolyl | N-tolyl | methyl | methyl | b6 | b6 |
| 2-2-380 | N-tolyl | N-tolyl | b1 | b1 | b6 | b6 |
| 2-2-381 | N-tolyl | N-tolyl | b2 | b2 | b6 | b6 |
| 2-2-382 | N-tolyl | N-tolyl | b3 | b3 | b6 | b6 |
| 2-2-383 | N-tolyl | N-tolyl | H | H | b7 | b7 |
| 2-2-384 | N-tolyl | N-tolyl | t-bu | t-bu | b7 | b7 |
| 2-2-385 | N-tolyl | N-tolyl | methyl | methyl | b7 | b7 |
| 2-2-386 | N-tolyl | N-tolyl | b1 | b1 | b7 | b7 |
| 2-2-387 | N-tolyl | N-tolyl | b2 | b2 | b7 | b7 |
| 2-2-388 | N-tolyl | N-tolyl | b3 | b3 | b7 | b7 |
| 2-2-389 | N-Biph | N-Biph | H | H | H | H |
| 2-2-390 | N-Biph | N-Biph | t-bu | t-bu | H | H |
| 2-2-391 | N-Biph | N-Biph | methyl | methyl | H | H |
| 2-2-392 | N-Biph | N-Biph | t-bu | t-bu | t-bu | t-bu |
| 2-2-393 | N-Biph | N-Biph | methyl | methyl | methyl | methyl |
| 2-2-394 | N-Biph | N-Biph | b1 | b1 | H | H |
| 2-2-395 | N-Biph | N-Biph | b2 | b2 | H | H |
| 2-2-396 | N-Biph | N-Biph | b3 | b3 | H | H |
| 2-2-397 | N-Biph | N-Biph | H | H | H | a1 |
| 2-2-398 | N-Biph | N-Biph | t-bu | t-bu | H | a1 |
| 2-2-399 | N-Biph | N-Biph | methyl | methyl | H | a1 |
| 2-2-400 | N-Biph | N-Biph | b1 | b1 | H | a1 |
| 2-2-401 | N-Biph | N-Biph | b2 | b2 | H | a1 |
| 2-2-402 | N-Biph | N-Biph | b3 | b3 | H | a1 |
| 2-2-403 | N-Biph | N-Biph | H | H | H | a2 |
| 2-2-404 | N-Biph | N-Biph | t-bu | t-bu | H | a2 |
| 2-2-405 | N-Biph | N-Biph | methyl | methyl | H | a2 |
| 2-2-406 | N-Biph | N-Biph | b1 | b1 | H | a2 |
| 2-2-407 | N-Biph | N-Biph | b2 | b2 | H | a2 |
| 2-2-408 | N-Biph | N-Biph | b3 | b3 | H | a2 |
| 2-2-409 | N-Biph | N-Biph | H | H | H | a3 |
| 2-2-410 | N-Biph | N-Biph | t-bu | t-bu | H | a3 |
| 2-2-411 | N-Biph | N-Biph | methyl | methyl | H | a3 |
| 2-2-412 | N-Biph | N-Biph | b1 | b1 | H | a3 |
| 2-2-413 | N-Biph | N-Biph | b2 | b2 | H | a3 |
| 2-2-414 | N-Biph | N-Biph | b3 | b3 | H | a3 |
| 2-2-415 | N-Biph | N-Biph | H | H | H | a4 |
| 2-2-416 | N-Biph | N-Biph | t-bu | t-bu | H | a4 |
| 2-2-417 | N-Biph | N-Biph | methyl | methyl | H | a4 |
| 2-2-418 | N-Biph | N-Biph | b1 | b1 | H | a4 |
| 2-2-419 | N-Biph | N-Biph | b2 | b2 | H | a4 |
| 2-2-420 | N-Biph | N-Biph | b3 | b3 | H | a4 |
| 2-2-421 | N-Biph | N-Biph | H | H | H | a5 |
| 2-2-422 | N-Biph | N-Biph | t-bu | t-bu | H | a5 |
| 2-2-423 | N-Biph | N-Biph | methyl | methyl | H | a5 |
| 2-2-424 | N-Biph | N-Biph | b1 | b1 | H | a5 |
| 2-2-425 | N-Biph | N-Biph | b2 | b2 | H | a5 |
| 2-2-426 | N-Biph | N-Biph | b3 | b3 | H | a5 |
| 2-2-427 | N-Biph | N-Biph | H | H | H | a6 |
| 2-2-428 | N-Biph | N-Biph | t-bu | t-bu | H | a6 |
| 2-2-429 | N-Biph | N-Biph | methyl | methyl | H | a6 |
| 2-2-430 | N-Biph | N-Biph | b1 | b1 | H | a6 |
| 2-2-431 | N-Biph | N-Biph | b2 | b2 | H | a6 |
| 2-2-432 | N-Biph | N-Biph | b3 | b3 | H | a6 |
| 2-2-433 | N-Biph | N-Biph | H | H | a1 | H |
| 2-2-434 | N-Biph | N-Biph | t-bu | t-bu | a1 | H |
| 2-2-435 | N-Biph | N-Biph | methyl | methyl | a1 | H |
| 2-2-436 | N-Biph | N-Biph | b1 | b1 | a1 | H |
| 2-2-437 | N-Biph | N-Biph | b2 | b2 | a1 | H |
| 2-2-438 | N-Biph | N-Biph | b3 | b3 | a1 | H |
| 2-2-439 | N-Biph | N-Biph | H | H | a2 | H |
| 2-2-440 | N-Biph | N-Biph | t-bu | t-bu | a2 | H |
| 2-2-441 | N-Biph | N-Biph | methyl | methyl | a2 | H |
| 2-2-442 | N-Biph | N-Biph | b1 | b1 | a2 | H |
| 2-2-443 | N-Biph | N-Biph | b2 | b2 | a2 | H |
| 2-2-444 | N-Biph | N-Biph | b3 | b3 | a2 | H |
| 2-2-445 | N-Biph | N-Biph | H | H | a3 | H |
| 2-2-446 | N-Biph | N-Biph | t-bu | t-bu | a3 | H |
| 2-2-447 | N-Biph | N-Biph | methyl | methyl | a3 | H |
| 2-2-448 | N-Biph | N-Biph | b1 | b1 | a3 | H |
| 2-2-449 | N-Biph | N-Biph | b2 | b2 | a3 | H |
| 2-2-450 | N-Biph | N-Biph | b3 | b3 | a3 | H |
| 2-2-451 | N-Biph | N-Biph | H | H | a4 | H |
| 2-2-452 | N-Biph | N-Biph | t-bu | t-bu | a4 | H |
| 2-2-453 | N-Biph | N-Biph | methyl | methyl | a4 | H |
| 2-2-454 | N-Biph | N-Biph | b1 | b1 | a4 | H |
| 2-2-455 | N-Biph | N-Biph | b2 | b2 | a4 | H |
| 2-2-456 | N-Biph | N-Biph | b3 | b3 | a4 | H |
| 2-2-457 | N-Biph | N-Biph | H | H | a5 | H |
| 2-2-458 | N-Biph | N-Biph | t-bu | t-bu | a5 | H |
| 2-2-459 | N-Biph | N-Biph | methyl | methyl | a5 | H |
| 2-2-460 | N-Biph | N-Biph | b1 | b1 | a5 | H |
| 2-2-461 | N-Biph | N-Biph | b2 | b2 | a5 | H |
| 2-2-462 | N-Biph | N-Biph | b3 | b3 | a5 | H |
| 2-2-463 | N-Biph | N-Biph | H | H | a6 | H |
| 2-2-464 | N-Biph | N-Biph | t-bu | t-bu | a6 | H |
| 2-2-465 | N-Biph | N-Biph | methyl | methyl | a6 | H |
| 2-2-466 | N-Biph | N-Biph | b1 | b1 | a6 | H |
| 2-2-467 | N-Biph | N-Biph | b2 | b2 | a6 | H |
| 2-2-468 | N-Biph | N-Biph | b3 | b3 | a6 | H |
| 2-2-469 | N-Biph | N-Biph | H | H | b1 | H |
| 2-2-470 | N-Biph | N-Biph | t-bu | t-bu | b1 | H |
| 2-2-471 | N-Biph | N-Biph | methyl | methyl | b1 | H |
| 2-2-472 | N-Biph | N-Biph | b1 | b1 | b1 | H |
| 2-2-473 | N-Biph | N-Biph | b2 | b2 | b1 | H |
| 2-2-474 | N-Biph | N-Biph | b3 | b3 | b1 | H |
| 2-2-475 | N-Biph | N-Biph | H | H | b2 | H |
| 2-2-476 | N-Biph | N-Biph | t-bu | t-bu | b2 | H |
| 2-2-477 | N-Biph | N-Biph | methyl | methyl | b2 | H |
| 2-2-478 | N-Biph | N-Biph | b1 | b1 | b2 | H |
| 2-2-479 | N-Biph | N-Biph | b2 | b2 | b2 | H |
| 2-2-480 | N-Biph | N-Biph | b3 | b3 | b2 | H |
| 2-2-481 | N-Biph | N-Biph | H | H | b3 | H |
| 2-2-482 | N-Biph | N-Biph | t-bu | t-bu | b3 | H |
| 2-2-483 | N-Biph | N-Biph | methyl | methyl | b3 | H |
| 2-2-484 | N-Biph | N-Biph | b1 | b1 | b3 | H |
| 2-2-485 | N-Biph | N-Biph | b2 | b2 | b3 | H |
| 2-2-486 | N-Biph | N-Biph | b3 | b3 | b3 | H |
| 2-2-487 | N-Biph | N-Biph | H | H | b4 | H |
| 2-2-488 | N-Biph | N-Biph | t-bu | t-bu | b4 | H |
| 2-2-489 | N-Biph | N-Biph | methyl | methyl | b4 | H |
| 2-2-490 | N-Biph | N-Biph | b1 | b1 | b4 | H |
| 2-2-491 | N-Biph | N-Biph | b2 | b2 | b4 | H |
| 2-2-492 | N-Biph | N-Biph | b3 | b3 | b4 | H |
| 2-2-493 | N-Biph | N-Biph | H | H | b5 | H |
| 2-2-494 | N-Biph | N-Biph | t-bu | t-bu | b5 | H |
| 2-2-495 | N-Biph | N-Biph | methyl | methyl | b5 | H |
| 2-2-496 | N-Biph | N-Biph | b1 | b1 | b5 | H |
| 2-2-497 | N-Biph | N-Biph | b2 | b2 | b5 | H |
| 2-2-498 | N-Biph | N-Biph | b3 | b3 | b5 | H |
| 2-2-499 | N-Biph | N-Biph | H | H | b6 | H |
| 2-2-500 | N-Biph | N-Biph | t-bu | t-bu | b6 | H |
| 2-2-501 | N-Biph | N-Biph | methyl | methyl | b6 | H |
| 2-2-502 | N-Biph | N-Biph | b1 | b1 | b6 | H |
| 2-2-503 | N-Biph | N-Biph | b2 | b2 | b6 | H |
| 2-2-504 | N-Biph | N-Biph | b3 | b3 | b6 | H |
| 2-2-505 | N-Biph | N-Biph | H | H | b7 | H |
| 2-2-506 | N-Biph | N-Biph | t-bu | t-bu | b7 | H |
| 2-2-507 | N-Biph | N-Biph | methyl | methyl | b7 | H |
| 2-2-508 | N-Biph | N-Biph | b1 | b1 | b7 | H |
| 2-2-509 | N-Biph | N-Biph | b2 | b2 | b7 | H |
| 2-2-510 | N-Biph | N-Biph | b3 | b3 | b7 | H |
| 2-2-511 | N-Biph | N-Biph | H | H | b8 | H |
| 2-2-512 | N-Biph | N-Biph | t-bu | t-bu | b8 | H |
| 2-2-513 | N-Biph | N-Biph | methyl | methyl | b8 | H |
| 2-2-514 | N-Biph | N-Biph | b1 | b1 | b8 | H |
| 2-2-515 | N-Biph | N-Biph | b2 | b2 | b8 | H |
| 2-2-516 | N-Biph | N-Biph | b3 | b3 | b8 | H |
| 2-2-517 | N-Biph | N-Biph | H | H | b9 | H |
| 2-2-518 | N-Biph | N-Biph | t-bu | t-bu | b9 | H |
| 2-2-519 | N-Biph | N-Biph | methyl | methyl | b9 | H |
| 2-2-520 | N-Biph | N-Biph | b1 | b1 | b9 | H |
| 2-2-521 | N-Biph | N-Biph | b2 | b2 | b9 | H |
| 2-2-522 | N-Biph | N-Biph | b3 | b3 | b9 | H |
| 2-2-523 | N-Biph | N-Biph | H | H | b10 | H |
| 2-2-524 | N-Biph | N-Biph | t-bu | t-bu | b10 | H |
| 2-2-525 | N-Biph | N-Biph | methyl | methyl | b10 | H |
| 2-2-526 | N-Biph | N-Biph | b1 | b1 | b10 | H |
| 2-2-527 | N-Biph | N-Biph | b2 | b2 | b10 | H |
| 2-2-528 | N-Biph | N-Biph | b3 | b3 | b10 | H |
| 2-2-529 | N-Biph | N-Biph | H | H | a1 | a1 |
| 2-2-530 | N-Biph | N-Biph | t-bu | t-bu | a1 | a1 |
| 2-2-531 | N-Biph | N-Biph | methyl | methyl | a1 | a1 |
| 2-2-532 | N-Biph | N-Biph | b1 | b1 | a1 | a1 |
| 2-2-533 | N-Biph | N-Biph | b2 | b2 | a1 | a1 |
| 2-2-534 | N-Biph | N-Biph | b3 | b3 | a1 | a1 |
| 2-2-535 | N-Biph | N-Biph | H | H | a2 | a2 |
| 2-2-536 | N-Biph | N-Biph | t-bu | t-bu | a2 | a2 |
| 2-2-537 | N-Biph | N-Biph | methyl | methyl | a2 | a2 |
| 2-2-538 | N-Biph | N-Biph | b1 | b1 | a2 | a2 |
| 2-2-539 | N-Biph | N-Biph | b2 | b2 | a2 | a2 |
| 2-2-540 | N-Biph | N-Biph | b3 | b3 | a2 | a2 |
| 2-2-541 | N-Biph | N-Biph | H | H | a3 | a3 |
| 2-2-542 | N-Biph | N-Biph | t-bu | t-bu | a3 | a3 |
| 2-2-543 | N-Biph | N-Biph | methyl | methyl | a3 | a3 |
| 2-2-544 | N-Biph | N-Biph | b1 | b1 | a3 | a3 |
| 2-2-545 | N-Biph | N-Biph | b2 | b2 | a3 | a3 |
| 2-2-546 | N-Biph | N-Biph | b3 | b3 | a3 | a3 |
| 2-2-547 | N-Biph | N-Biph | H | H | b1 | b1 |
| 2-2-548 | N-Biph | N-Biph | t-bu | t-bu | b1 | b1 |
| 2-2-549 | N-Biph | N-Biph | methyl | methyl | b1 | b1 |
| 2-2-550 | N-Biph | N-Biph | b1 | b1 | b1 | b1 |
| 2-2-551 | N-Biph | N-Biph | b2 | b2 | b1 | b1 |
| 2-2-552 | N-Biph | N-Biph | b3 | b3 | b1 | b1 |
| 2-2-553 | N-Biph | N-Biph | H | H | b2 | b2 |
| 2-2-554 | N-Biph | N-Biph | t-bu | t-bu | b2 | b2 |
| 2-2-555 | N-Biph | N-Biph | methyl | methyl | b2 | b2 |
| 2-2-556 | N-Biph | N-Biph | b1 | b1 | b2 | b2 |
| 2-2-557 | N-Biph | N-Biph | b2 | b2 | b2 | b2 |
| 2-2-558 | N-Biph | N-Biph | b3 | b3 | b2 | b2 |
| 2-2-559 | N-Biph | N-Biph | H | H | b3 | b3 |
| 2-2-560 | N-Biph | N-Biph | t-bu | t-bu | b3 | b3 |
| 2-2-561 | N-Biph | N-Biph | methyl | methyl | b3 | b3 |
| 2-2-562 | N-Biph | N-Biph | b1 | b1 | b3 | b3 |
| 2-2-563 | N-Biph | N-Biph | b2 | b2 | b3 | b3 |
| 2-2-564 | N-Biph | N-Biph | b3 | b3 | b3 | b3 |
| 2-2-565 | N-Biph | N-Biph | H | H | b5 | b5 |
| 2-2-566 | N-Biph | N-Biph | t-bu | t-bu | b5 | b5 |
| 2-2-567 | N-Biph | N-Biph | methyl | methyl | b5 | b5 |
| 2-2-568 | N-Biph | N-Biph | b1 | b1 | b5 | b5 |
| 2-2-569 | N-Biph | N-Biph | b2 | b2 | b5 | b5 |
| 2-2-570 | N-Biph | N-Biph | b3 | b3 | b5 | b5 |
| 2-2-571 | N-Biph | N-Biph | H | H | b6 | b6 |
| 2-2-572 | N-Biph | N-Biph | t-bu | t-bu | b6 | b6 |
| 2-2-573 | N-Biph | N-Biph | methyl | methyl | b6 | b6 |
| 2-2-574 | N-Biph | N-Biph | b1 | b1 | b6 | b6 |
| 2-2-575 | N-Biph | N-Biph | b2 | b2 | b6 | b6 |
| 2-2-576 | N-Biph | N-Biph | b3 | b3 | b6 | b6 |
| 2-2-577 | N-Biph | N-Biph | H | H | b7 | b7 |
| 2-2-578 | N-Biph | N-Biph | t-bu | t-bu | b7 | b7 |
| 2-2-579 | N-Biph | N-Biph | methyl | methyl | b7 | b7 |
| 2-2-580 | N-Biph | N-Biph | b1 | b1 | b7 | b7 |
| 2-2-581 | N-Biph | N-Biph | b2 | b2 | b7 | b7 |
| 2-2-582 | N-Biph | N-Biph | b3 | b3 | b7 | b7 |
| 2-2-583 | O | N—Ph | H | H | H | H |
| 2-2-584 | O | N—Ph | t-bu | t-bu | H | H |
| 2-2-585 | O | N—Ph | methyl | methyl | H | H |
| 2-2-586 | O | N—Ph | t-bu | t-bu | t-bu | t-bu |
| 2-2-587 | O | N—Ph | methyl | methyl | methyl | methyl |
| 2-2-588 | O | N—Ph | b1 | b1 | H | H |
| 2-2-589 | O | N—Ph | b2 | b2 | H | H |
| 2-2-590 | O | N—Ph | b3 | b3 | H | H |
| 2-2-591 | O | N—Ph | H | H | H | a1 |
| 2-2-592 | O | N—Ph | t-bu | t-bu | H | a1 |
| 2-2-593 | O | N—Ph | methyl | methyl | H | a1 |
| 2-2-594 | O | N—Ph | b1 | b1 | H | a1 |
| 2-2-595 | O | N—Ph | b2 | b2 | H | a1 |
| 2-2-596 | O | N—Ph | b3 | b3 | H | a1 |
| 2-2-597 | O | N—Ph | H | H | H | a2 |
| 2-2-598 | O | N—Ph | t-bu | t-bu | H | a2 |
| 2-2-599 | O | N—Ph | methyl | methyl | H | a2 |
| 2-2-600 | O | N—Ph | b1 | b1 | H | a2 |
| 2-2-601 | O | N—Ph | b2 | b2 | H | a2 |
| 2-2-602 | O | N—Ph | b3 | b3 | H | a2 |
| 2-2-603 | O | N—Ph | H | H | H | a3 |
| 2-2-604 | O | N—Ph | t-bu | t-bu | H | a3 |
| 2-2-605 | O | N—Ph | methyl | methyl | H | a3 |
| 2-2-606 | O | N—Ph | b1 | b1 | H | a3 |
| 2-2-607 | O | N—Ph | b2 | b2 | H | a3 |
| 2-2-608 | O | N—Ph | b3 | b3 | H | a3 |
| 2-2-609 | O | N—Ph | H | H | H | a4 |
| 2-2-610 | O | N—Ph | t-bu | t-bu | H | a4 |
| 2-2-611 | O | N—Ph | methyl | methyl | H | a4 |
| 2-2-612 | O | N—Ph | b1 | b1 | H | a4 |
| 2-2-613 | O | N—Ph | b2 | b2 | H | a4 |
| 2-2-614 | O | N—Ph | b3 | b3 | H | a4 |
| 2-2-615 | O | N—Ph | H | H | H | a5 |
| 2-2-616 | O | N—Ph | t-bu | t-bu | H | a5 |
| 2-2-617 | O | N—Ph | methyl | methyl | H | a5 |
| 2-2-618 | O | N—Ph | b1 | b1 | H | a5 |
| 2-2-619 | O | N—Ph | b2 | b2 | H | a5 |
| 2-2-620 | O | N—Ph | b3 | b3 | H | a5 |
| 2-2-621 | O | N—Ph | H | H | H | a6 |
| 2-2-622 | O | N—Ph | t-bu | t-bu | H | a6 |
| 2-2-623 | O | N—Ph | methyl | methyl | H | a6 |
| 2-2-624 | O | N—Ph | b1 | b1 | H | a6 |
| 2-2-625 | O | N—Ph | b2 | b2 | H | a6 |
| 2-2-626 | O | N—Ph | b3 | b3 | H | a6 |
| 2-2-627 | O | N—Ph | H | H | a1 | H |
| 2-2-628 | O | N—Ph | t-bu | t-bu | a1 | H |
| 2-2-629 | O | N—Ph | methyl | methyl | a1 | H |
| 2-2-630 | O | N—Ph | b1 | b1 | a1 | H |
| 2-2-631 | O | N—Ph | b2 | b2 | a1 | H |
| 2-2-632 | O | N—Ph | b3 | b3 | a1 | H |
| 2-2-633 | O | N—Ph | H | H | a2 | H |
| 2-2-634 | O | N—Ph | t-bu | t-bu | a2 | H |
| 2-2-635 | O | N—Ph | methyl | methyl | a2 | H |
| 2-2-636 | O | N—Ph | b1 | b1 | a2 | H |
| 2-2-637 | O | N—Ph | b2 | b2 | a2 | H |
| 2-2-638 | O | N—Ph | b3 | b3 | a2 | H |
| 2-2-639 | O | N—Ph | H | H | a3 | H |
| 2-2-640 | O | N—Ph | t-bu | t-bu | a3 | H |
| 2-2-641 | O | N—Ph | methyl | methyl | a3 | H |
| 2-2-642 | O | N—Ph | b1 | b1 | a3 | H |
| 2-2-643 | O | N—Ph | b2 | b2 | a3 | H |
| 2-2-644 | O | N—Ph | b3 | b3 | a3 | H |
| 2-2-645 | O | N—Ph | H | H | a4 | H |
| 2-2-646 | O | N—Ph | t-bu | t-bu | a4 | H |
| 2-2-647 | O | N—Ph | methyl | methyl | a4 | H |
| 2-2-648 | O | N—Ph | b1 | b1 | a4 | H |
| 2-2-649 | O | N—Ph | b2 | b2 | a4 | H |
| 2-2-650 | O | N—Ph | b3 | b3 | a4 | H |
| 2-2-651 | O | N—Ph | H | H | a5 | H |
| 2-2-652 | O | N—Ph | t-bu | t-bu | a5 | H |
| 2-2-653 | O | N—Ph | methyl | methyl | a5 | H |
| 2-2-654 | O | N—Ph | b1 | b1 | a5 | H |
| 2-2-655 | O | N—Ph | b2 | b2 | a5 | H |
| 2-2-656 | O | N—Ph | b3 | b3 | a5 | H |
| 2-2-657 | O | N—Ph | H | H | a6 | H |
| 2-2-658 | O | N—Ph | t-bu | t-bu | a6 | H |
| 2-2-659 | O | N—Ph | methyl | methyl | a6 | H |
| 2-2-660 | O | N—Ph | b1 | b1 | a6 | H |
| 2-2-661 | O | N—Ph | b2 | b2 | a6 | H |
| 2-2-662 | O | N—Ph | b3 | b3 | a6 | H |
| 2-2-663 | O | N—Ph | H | H | b1 | H |
| 2-2-664 | O | N—Ph | t-bu | t-bu | b1 | H |
| 2-2-665 | O | N—Ph | methyl | methyl | b1 | H |
| 2-2-666 | O | N—Ph | b1 | b1 | b1 | H |
| 2-2-667 | O | N—Ph | b2 | b2 | b1 | H |
| 2-2-668 | O | N—Ph | b3 | b3 | b1 | H |
| 2-2-669 | O | N—Ph | H | H | b2 | H |
| 2-2-670 | O | N—Ph | t-bu | t-bu | b2 | H |
| 2-2-671 | O | N—Ph | methyl | methyl | b2 | H |
| 2-2-672 | O | N—Ph | b1 | b1 | b2 | H |
| 2-2-673 | O | N—Ph | b2 | b2 | b2 | H |
| 2-2-674 | O | N—Ph | b3 | b3 | b2 | H |
| 2-2-675 | O | N—Ph | H | H | b3 | H |
| 2-2-676 | O | N—Ph | t-bu | t-bu | b3 | H |
| 2-2-677 | O | N—Ph | methyl | methyl | b3 | H |
| 2-2-678 | O | N—Ph | b1 | b1 | b3 | H |
| 2-2-679 | O | N—Ph | b2 | b2 | b3 | H |
| 2-2-680 | O | N—Ph | b3 | b3 | b3 | H |
| 2-2-681 | O | N—Ph | H | H | b4 | H |
| 2-2-682 | O | N—Ph | t-bu | t-bu | b4 | H |
| 2-2-683 | O | N—Ph | methyl | methyl | b4 | H |
| 2-2-684 | O | N—Ph | b1 | b1 | b4 | H |
| 2-2-685 | O | N—Ph | b2 | b2 | b4 | H |
| 2-2-686 | O | N—Ph | b3 | b3 | b4 | H |
| 2-2-687 | O | N—Ph | H | H | b5 | H |
| 2-2-688 | O | N—Ph | t-bu | t-bu | b5 | H |
| 2-2-689 | O | N—Ph | methyl | methyl | b5 | H |
| 2-2-690 | O | N—Ph | b1 | b1 | b5 | H |
| 2-2-691 | O | N—Ph | b2 | b2 | b5 | H |
| 2-2-692 | O | N—Ph | b3 | b3 | b5 | H |
| 2-2-693 | O | N—Ph | H | H | b6 | H |
| 2-2-694 | O | N—Ph | t-bu | t-bu | b6 | H |
| 2-2-695 | O | N—Ph | methyl | methyl | b6 | H |
| 2-2-696 | O | N—Ph | b1 | b1 | b6 | H |
| 2-2-697 | O | N—Ph | b2 | b2 | b6 | H |
| 2-2-698 | O | N—Ph | b3 | b3 | b6 | H |
| 2-2-699 | O | N—Ph | H | H | b7 | H |
| 2-2-700 | O | N—Ph | t-bu | t-bu | b7 | H |
| 2-2-701 | O | N—Ph | methyl | methyl | b7 | H |
| 2-2-702 | O | N—Ph | b1 | b1 | b7 | H |
| 2-2-703 | O | N—Ph | b2 | b2 | b7 | H |
| 2-2-704 | O | N—Ph | b3 | b3 | b7 | H |
| 2-2-705 | O | N—Ph | H | H | b8 | H |
| 2-2-706 | O | N—Ph | t-bu | t-bu | b8 | H |
| 2-2-707 | O | N—Ph | methyl | methyl | b8 | H |
| 2-2-708 | O | N—Ph | b1 | b1 | b8 | H |
| 2-2-709 | O | N—Ph | b2 | b2 | b8 | H |
| 2-2-710 | O | N—Ph | b3 | b3 | b8 | H |
| 2-2-711 | O | N—Ph | H | H | b9 | H |
| 2-2-712 | O | N—Ph | t-bu | t-bu | b9 | H |
| 2-2-713 | O | N—Ph | methyl | methyl | b9 | H |
| 2-2-714 | O | N—Ph | b1 | b1 | b9 | H |
| 2-2-715 | O | N—Ph | b2 | b2 | b9 | H |
| 2-2-716 | O | N—Ph | b3 | b3 | b9 | H |
| 2-2-717 | O | N—Ph | H | H | b10 | H |
| 2-2-718 | O | N—Ph | t-bu | t-bu | b10 | H |
| 2-2-719 | O | N—Ph | methyl | methyl | b10 | H |
| 2-2-720 | O | N—Ph | b1 | b1 | b10 | H |
| 2-2-721 | O | N—Ph | b2 | b2 | b10 | H |
| 2-2-722 | O | N—Ph | b3 | b3 | b10 | H |
| 2-2-723 | O | N—Ph | H | H | a1 | a1 |
| 2-2-724 | O | N—Ph | t-bu | t-bu | a1 | a1 |
| 2-2-725 | O | N—Ph | methyl | methyl | a1 | a1 |
| 2-2-726 | O | N—Ph | b1 | b1 | a1 | a1 |
| 2-2-727 | O | N—Ph | b2 | b2 | a1 | a1 |
| 2-2-728 | O | N—Ph | b3 | b3 | a1 | a1 |
| 2-2-729 | O | N—Ph | H | H | a2 | a2 |
| 2-2-730 | O | N—Ph | t-bu | t-bu | a2 | a2 |
| 2-2-731 | O | N—Ph | methyl | methyl | a2 | a2 |
| 2-2-732 | O | N—Ph | b1 | b1 | a2 | a2 |
| 2-2-733 | O | N—Ph | b2 | b2 | a2 | a2 |
| 2-2-734 | O | N—Ph | b3 | b3 | a2 | a2 |
| 2-2-735 | O | N—Ph | H | H | a3 | a3 |
| 2-2-736 | O | N—Ph | t-bu | t-bu | a3 | a3 |
| 2-2-737 | O | N—Ph | methyl | methyl | a3 | a3 |
| 2-2-738 | O | N—Ph | b1 | b1 | a3 | a3 |
| 2-2-739 | O | N—Ph | b2 | b2 | a3 | a3 |
| 2-2-740 | O | N—Ph | b3 | b3 | a3 | a3 |
| 2-2-741 | O | N—Ph | H | H | b1 | b1 |
| 2-2-742 | O | N—Ph | t-bu | t-bu | b1 | b1 |
| 2-2-743 | O | N—Ph | methyl | methyl | b1 | b1 |
| 2-2-744 | O | N—Ph | b1 | b1 | b1 | b1 |
| 2-2-745 | O | N—Ph | b2 | b2 | b1 | b1 |
| 2-2-746 | O | N—Ph | b3 | b3 | b1 | b1 |
| 2-2-747 | O | N—Ph | H | H | b2 | b2 |
| 2-2-748 | O | N—Ph | t-bu | t-bu | b2 | b2 |
| 2-2-749 | O | N—Ph | methyl | methyl | b2 | b2 |
| 2-2-750 | O | N—Ph | b1 | b1 | b2 | b2 |
| 2-2-751 | O | N—Ph | b2 | b2 | b2 | b2 |
| 2-2-752 | O | N—Ph | b3 | b3 | b2 | b2 |
| 2-2-753 | O | N—Ph | H | H | b3 | b3 |
| 2-2-754 | O | N—Ph | t-bu | t-bu | b3 | b3 |
| 2-2-755 | O | N—Ph | methyl | methyl | b3 | b3 |
| 2-2-756 | O | N—Ph | b1 | b1 | b3 | b3 |
| 2-2-757 | O | N—Ph | b2 | b2 | b3 | b3 |
| 2-2-758 | O | N—Ph | b3 | b3 | b3 | b3 |
| 2-2-759 | O | N—Ph | H | H | b5 | b5 |
| 2-2-760 | O | N—Ph | t-bu | t-bu | b5 | b5 |
| 2-2-761 | O | N—Ph | methyl | methyl | b5 | b5 |
| 2-2-762 | O | N—Ph | b1 | b1 | b5 | b5 |
| 2-2-763 | O | N—Ph | b2 | b2 | b5 | b5 |
| 2-2-764 | O | N—Ph | b3 | b3 | b5 | b5 |
| 2-2-765 | O | N—Ph | H | H | b6 | b6 |
| 2-2-766 | O | N—Ph | t-bu | t-bu | b6 | b6 |
| 2-2-767 | O | N—Ph | methyl | methyl | b6 | b6 |
| 2-2-768 | O | N—Ph | b1 | b1 | b6 | b6 |
| 2-2-769 | O | N—Ph | b2 | b2 | b6 | b6 |
| 2-2-770 | O | N—Ph | b3 | b3 | b6 | b6 |
| 2-2-771 | O | N—Ph | H | H | b7 | b7 |
| 2-2-772 | O | N—Ph | t-bu | t-bu | b7 | b7 |
| 2-2-773 | O | N—Ph | methyl | methyl | b7 | b7 |
| 2-2-774 | O | N—Ph | b1 | b1 | b7 | b7 |
| 2-2-775 | O | N—Ph | b2 | b2 | b7 | b7 |
| 2-2-776 | O | N—Ph | b3 | b3 | b7 | b7 |
| 2-2-777 | S | N—Ph | H | H | H | H |
| 2-2-778 | S | N—Ph | t-bu | t-bu | H | H |
| 2-2-779 | S | N—Ph | methyl | methyl | H | H |
| 2-2-780 | S | N—Ph | t-bu | t-bu | t-bu | t-bu |
| 2-2-781 | S | N—Ph | methyl | methyl | methyl | methyl |
| 2-2-782 | S | N—Ph | b1 | b1 | H | H |
| 2-2-783 | S | N—Ph | b2 | b2 | H | H |
| 2-2-784 | S | N—Ph | b3 | b3 | H | H |
| 2-2-785 | S | N—Ph | H | H | H | a1 |
| 2-2-786 | S | N—Ph | t-bu | t-bu | H | a1 |
| 2-2-787 | S | N—Ph | methyl | methyl | H | a1 |
| 2-2-788 | S | N—Ph | b1 | b1 | H | a1 |
| 2-2-789 | S | N—Ph | b2 | b2 | H | a1 |
| 2-2-790 | S | N—Ph | b3 | b3 | H | a1 |
| 2-2-791 | S | N—Ph | H | H | H | a2 |
| 2-2-792 | S | N—Ph | t-bu | t-bu | H | a2 |
| 2-2-793 | S | N—Ph | methyl | methyl | H | a2 |
| 2-2-794 | S | N—Ph | b1 | b1 | H | a2 |
| 2-2-795 | S | N—Ph | b2 | b2 | H | a2 |
| 2-2-796 | S | N—Ph | b3 | b3 | H | a2 |
| 2-2-797 | S | N—Ph | H | H | H | a3 |
| 2-2-798 | S | N—Ph | t-bu | t-bu | H | a3 |
| 2-2-799 | S | N—Ph | methyl | methyl | H | a3 |
| 2-2-800 | S | N—Ph | b1 | b1 | H | a3 |
| 2-2-801 | S | N—Ph | b2 | b2 | H | a3 |
| 2-2-802 | S | N—Ph | b3 | b3 | H | a3 |
| 2-2-803 | S | N—Ph | H | H | H | a4 |
| 2-2-804 | S | N—Ph | t-bu | t-bu | H | a4 |
| 2-2-805 | S | N—Ph | methyl | methyl | H | a4 |
| 2-2-806 | S | N—Ph | b1 | b1 | H | a4 |
| 2-2-807 | S | N—Ph | b2 | b2 | H | a4 |
| 2-2-808 | S | N—Ph | b3 | b3 | H | a4 |
| 2-2-809 | S | N—Ph | H | H | H | a5 |
| 2-2-810 | S | N—Ph | t-bu | t-bu | H | a5 |
| 2-2-811 | S | N—Ph | methyl | methyl | H | a5 |
| 2-2-812 | S | N—Ph | b1 | b1 | H | a5 |
| 2-2-813 | S | N—Ph | b2 | b2 | H | a5 |
| 2-2-814 | S | N—Ph | b3 | b3 | H | a5 |
| 2-2-815 | S | N—Ph | H | H | H | a6 |
| 2-2-816 | S | N—Ph | t-bu | t-bu | H | a6 |
| 2-2-817 | S | N—Ph | methyl | methyl | H | a6 |
| 2-2-818 | S | N—Ph | b1 | b1 | H | a6 |
| 2-2-819 | S | N—Ph | b2 | b2 | H | a6 |
| 2-2-820 | S | N—Ph | b3 | b3 | H | a6 |
| 2-2-821 | S | N—Ph | H | H | a1 | H |
| 2-2-822 | S | N—Ph | t-bu | t-bu | a1 | H |
| 2-2-823 | S | N—Ph | methyl | methyl | a1 | H |
| 2-2-824 | S | N—Ph | b1 | b1 | a1 | H |
| 2-2-825 | S | N—Ph | b2 | b2 | a1 | H |
| 2-2-826 | S | N—Ph | b3 | b3 | a1 | H |
| 2-2-827 | S | N—Ph | H | H | a2 | H |
| 2-2-828 | S | N—Ph | t-bu | t-bu | a2 | H |
| 2-2-829 | S | N—Ph | methyl | methyl | a2 | H |
| 2-2-830 | S | N—Ph | b1 | b1 | a2 | H |
| 2-2-831 | S | N—Ph | b2 | b2 | a2 | H |
| 2-2-832 | S | N—Ph | b3 | b3 | a2 | H |
| 2-2-833 | S | N—Ph | H | H | a3 | H |
| 2-2-834 | S | N—Ph | t-bu | t-bu | a3 | H |
| 2-2-835 | S | N—Ph | methyl | methyl | a3 | H |
| 2-2-836 | S | N—Ph | b1 | b1 | a3 | H |
| 2-2-837 | S | N—Ph | b2 | b2 | a3 | H |
| 2-2-838 | S | N—Ph | b3 | b3 | a3 | H |
| 2-2-839 | S | N—Ph | H | H | a4 | H |
| 2-2-840 | S | N—Ph | t-bu | t-bu | a4 | H |
| 2-2-841 | S | N—Ph | methyl | methyl | a4 | H |
| 2-2-842 | S | N—Ph | b1 | b1 | a4 | H |
| 2-2-843 | S | N—Ph | b2 | b2 | a4 | H |
| 2-2-844 | S | N—Ph | b3 | b3 | a4 | H |
| 2-2-845 | S | N—Ph | H | H | a5 | H |
| 2-2-846 | S | N—Ph | t-bu | t-bu | a5 | H |
| 2-2-847 | S | N—Ph | methyl | methyl | a5 | H |
| 2-2-848 | S | N—Ph | b1 | b1 | a5 | H |
| 2-2-849 | S | N—Ph | b2 | b2 | a5 | H |
| 2-2-850 | S | N—Ph | b3 | b3 | a5 | H |
| 2-2-851 | S | N—Ph | H | H | a6 | H |
| 2-2-852 | S | N—Ph | t-bu | t-bu | a6 | H |
| 2-2-853 | S | N—Ph | methyl | methyl | a6 | H |
| 2-2-854 | S | N—Ph | b1 | b1 | a6 | H |
| 2-2-855 | S | N—Ph | b2 | b2 | a6 | H |
| 2-2-856 | S | N—Ph | b3 | b3 | a6 | H |
| 2-2-857 | S | N—Ph | H | H | b1 | H |
| 2-2-858 | S | N—Ph | t-bu | t-bu | b1 | H |
| 2-2-859 | S | N—Ph | methyl | methyl | b1 | H |
| 2-2-860 | S | N—Ph | b1 | b1 | b1 | H |
| 2-2-861 | S | N—Ph | b2 | b2 | b1 | H |
| 2-2-862 | S | N—Ph | b3 | b3 | b1 | H |
| 2-2-863 | S | N—Ph | H | H | b2 | H |
| 2-2-864 | S | N—Ph | t-bu | t-bu | b2 | H |
| 2-2-865 | S | N—Ph | methyl | methyl | b2 | H |
| 2-2-866 | S | N—Ph | b1 | b1 | b2 | H |
| 2-2-867 | S | N—Ph | b2 | b2 | b2 | H |
| 2-2-868 | S | N—Ph | b3 | b3 | b2 | H |
| 2-2-869 | S | N—Ph | H | H | b3 | H |
| 2-2-870 | S | N—Ph | t-bu | t-bu | b3 | H |
| 2-2-871 | S | N—Ph | methyl | methyl | b3 | H |
| 2-2-872 | S | N—Ph | b1 | b1 | b3 | H |
| 2-2-873 | S | N—Ph | b2 | b2 | b3 | H |
| 2-2-874 | S | N—Ph | b3 | b3 | b3 | H |
| 2-2-875 | S | N—Ph | H | H | b4 | H |
| 2-2-876 | S | N—Ph | t-bu | t-bu | b4 | H |
| 2-2-877 | S | N—Ph | methyl | methyl | b4 | H |
| 2-2-878 | S | N—Ph | b1 | b1 | b4 | H |
| 2-2-879 | S | N—Ph | b2 | b2 | b4 | H |
| 2-2-880 | S | N—Ph | b3 | b3 | b4 | H |
| 2-2-881 | S | N—Ph | H | H | b5 | H |
| 2-2-882 | S | N—Ph | t-bu | t-bu | b5 | H |
| 2-2-883 | S | N—Ph | methyl | methyl | b5 | H |
| 2-2-884 | S | N—Ph | b1 | b1 | b5 | H |
| 2-2-885 | S | N—Ph | b2 | b2 | b5 | H |
| 2-2-886 | S | N—Ph | b3 | b3 | b5 | H |
| 2-2-887 | S | N—Ph | H | H | b6 | H |
| 2-2-888 | S | N—Ph | t-bu | t-bu | b6 | H |
| 2-2-889 | S | N—Ph | methyl | methyl | b6 | H |
| 2-2-890 | S | N—Ph | b1 | b1 | b6 | H |
| 2-2-891 | S | N—Ph | b2 | b2 | b6 | H |
| 2-2-892 | S | N—Ph | b3 | b3 | b6 | H |
| 2-2-893 | S | N—Ph | H | H | b7 | H |
| 2-2-894 | S | N—Ph | t-bu | t-bu | b7 | H |
| 2-2-895 | S | N—Ph | methyl | methyl | b7 | H |
| 2-2-896 | S | N—Ph | b1 | b1 | b7 | H |
| 2-2-897 | S | N—Ph | b2 | b2 | b7 | H |
| 2-2-898 | S | N—Ph | b3 | b3 | b7 | H |
| 2-2-899 | S | N—Ph | H | H | b8 | H |
| 2-2-900 | S | N—Ph | t-bu | t-bu | b8 | H |
| 2-2-901 | S | N—Ph | methyl | methyl | b8 | H |
| 2-2-902 | S | N—Ph | b1 | b1 | b8 | H |
| 2-2-903 | S | N—Ph | b2 | b2 | b8 | H |
| 2-2-904 | S | N—Ph | b3 | b3 | b8 | H |
| 2-2-905 | S | N—Ph | H | H | b9 | H |
| 2-2-906 | S | N—Ph | t-bu | t-bu | b9 | H |
| 2-2-907 | S | N—Ph | methyl | methyl | b9 | H |
| 2-2-908 | S | N—Ph | b1 | b1 | b9 | H |
| 2-2-909 | S | N—Ph | b2 | b2 | b9 | H |
| 2-2-910 | S | N—Ph | b3 | b3 | b9 | H |
| 2-2-911 | S | N—Ph | H | H | b10 | H |
| 2-2-912 | S | N—Ph | t-bu | t-bu | b10 | H |
| 2-2-913 | S | N—Ph | methyl | methyl | b10 | H |
| 2-2-914 | S | N—Ph | b1 | b1 | b10 | H |
| 2-2-915 | S | N—Ph | b2 | b2 | b10 | H |
| 2-2-916 | S | N—Ph | b3 | b3 | b10 | H |
| 2-2-917 | S | N—Ph | H | H | a1 | a1 |
| 2-2-918 | S | N—Ph | t-bu | t-bu | a1 | a1 |
| 2-2-919 | S | N—Ph | methyl | methyl | a1 | a1 |
| 2-2-920 | S | N—Ph | b1 | b1 | a1 | a1 |
| 2-2-921 | S | N—Ph | b2 | b2 | a1 | a1 |
| 2-2-922 | S | N—Ph | b3 | b3 | a1 | a1 |
| 2-2-923 | S | N—Ph | H | H | a2 | a2 |
| 2-2-924 | S | N—Ph | t-bu | t-bu | a2 | a2 |
| 2-2-925 | S | N—Ph | methyl | methyl | a2 | a2 |
| 2-2-926 | S | N—Ph | b1 | b1 | a2 | a2 |
| 2-2-927 | S | N—Ph | b2 | b2 | a2 | a2 |
| 2-2-928 | S | N—Ph | b3 | b3 | a2 | a2 |
| 2-2-929 | S | N—Ph | H | H | a3 | a3 |
| 2-2-930 | S | N—Ph | t-bu | t-bu | a3 | a3 |
| 2-2-931 | S | N—Ph | methyl | methyl | a3 | a3 |
| 2-2-932 | S | N—Ph | b1 | b1 | a3 | a3 |
| 2-2-933 | S | N—Ph | b2 | b2 | a3 | a3 |
| 2-2-934 | S | N—Ph | b3 | b3 | a3 | a3 |
| 2-2-935 | S | N—Ph | H | H | b1 | b1 |
| 2-2-936 | S | N—Ph | t-bu | t-bu | b1 | b1 |
| 2-2-937 | S | N—Ph | methyl | methyl | b1 | b1 |
| 2-2-938 | S | N—Ph | b1 | b1 | b1 | b1 |
| 2-2-939 | S | N—Ph | b2 | b2 | b1 | b1 |
| 2-2-940 | S | N—Ph | b3 | b3 | b1 | b1 |
| 2-2-941 | S | N—Ph | H | H | b2 | b2 |
| 2-2-942 | S | N—Ph | t-bu | t-bu | b2 | b2 |
| 2-2-943 | S | N—Ph | methyl | methyl | b2 | b2 |
| 2-2-944 | S | N—Ph | b1 | b1 | b2 | b2 |
| 2-2-945 | S | N—Ph | b2 | b2 | b2 | b2 |
| 2-2-946 | S | N—Ph | b3 | b3 | b2 | b2 |
| 2-2-947 | S | N—Ph | H | H | b3 | b3 |
| 2-2-948 | S | N—Ph | t-bu | t-bu | b3 | b3 |
| 2-2-949 | S | N—Ph | methyl | methyl | b3 | b3 |
| 2-2-950 | S | N—Ph | b1 | b1 | b3 | b3 |
| 2-2-951 | S | N—Ph | b2 | b2 | b3 | b3 |
| 2-2-952 | S | N—Ph | b3 | b3 | b3 | b3 |
| 2-2-953 | S | N—Ph | H | H | b5 | b5 |
| 2-2-954 | S | N—Ph | t-bu | t-bu | b5 | b5 |
| 2-2-955 | S | N—Ph | methyl | methyl | b5 | b5 |
| 2-2-956 | S | N—Ph | b1 | b1 | b5 | b5 |
| 2-2-957 | S | N—Ph | b2 | b2 | b5 | b5 |
| 2-2-958 | S | N—Ph | b3 | b3 | b5 | b5 |
| 2-2-959 | S | N—Ph | H | H | b6 | b6 |
| 2-2-960 | S | N—Ph | t-bu | t-bu | b6 | b6 |
| 2-2-961 | S | N—Ph | methyl | methyl | b6 | b6 |
| 2-2-962 | S | N—Ph | b1 | b1 | b6 | b6 |
| 2-2-963 | S | N—Ph | b2 | b2 | b6 | b6 |
| 2-2-964 | S | N—Ph | b3 | b3 | b6 | b6 |
| 2-2-965 | S | N—Ph | H | H | b7 | b7 |
| 2-2-966 | S | N—Ph | t-bu | t-bu | b7 | b7 |
| 2-2-967 | S | N—Ph | methyl | methyl | b7 | b7 |
| 2-2-968 | S | N—Ph | b1 | b1 | b7 | b7 |
| 2-2-969 | S | N—Ph | b2 | b2 | b7 | b7 |
| 2-2-970 | S | N—Ph | b3 | b3 | b7 | b7 |

Group 2-3
The compounds are represented by the formula 2-3 below, wherein CY₁ and CY₃ are each a benzene group, X₁ is B, and Y1, Y2, R2b, R2d, (R1)a1, (R3)a3 are each shown in the table 3 below, 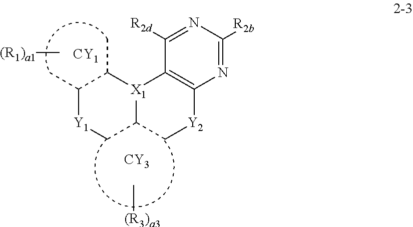
TABLE 3

| Compound | | | | | | |
|---|---|---|---|---|---|---|
| No. | Y1 | Y2 | R2b | R2d | (R1)a1 | (R3)a3 |
| 2-3-001 | N—Ph | N—Ph | H | H | H | H |
| 2-3-002 | N—Ph | N—Ph | t-bu | H | H | H |
| 2-3-003 | N—Ph | N—Ph | methyl | H | H | H |
| 2-3-004 | N—Ph | N—Ph | t-bu | t-bu | t-bu | t-bu |
| 2-3-005 | N—Ph | N—Ph | methyl | methyl | methyl | methyl |
| 2-3-006 | N—Ph | N—Ph | b1 | H | H | H |
| 2-3-007 | N—Ph | N—Ph | b2 | H | H | H |
| 2-3-008 | N—Ph | N—Ph | b3 | H | H | H |
| 2-3-009 | N—Ph | N—Ph | H | H | H | a1 |
| 2-3-010 | N—Ph | N—Ph | t-bu | H | H | a1 |
| 2-3-011 | N—Ph | N—Ph | methyl | H | H | a1 |
| 2-3-012 | N—Ph | N—Ph | b1 | H | H | a1 |
| 2-3-013 | N—Ph | N—Ph | b2 | H | H | a1 |
| 2-3-014 | N—Ph | N—Ph | b3 | H | H | a1 |
| 2-3-015 | N—Ph | N—Ph | H | H | H | a2 |
| 2-3-016 | N—Ph | N—Ph | t-bu | H | H | a2 |
| 2-3-017 | N—Ph | N—Ph | methyl | H | H | a2 |
| 2-3-018 | N—Ph | N—Ph | b1 | H | H | a2 |
| 2-3-019 | N—Ph | N—Ph | b2 | H | H | a2 |
| 2-3-020 | N—Ph | N—Ph | b3 | H | H | a2 |
| 2-3-021 | N—Ph | N—Ph | H | H | H | a3 |
| 2-3-022 | N—Ph | N—Ph | t-bu | H | H | a3 |
| 2-3-023 | N—Ph | N—Ph | methyl | H | H | a3 |
| 2-3-024 | N—Ph | N—Ph | b1 | H | H | a3 |
| 2-3-025 | N—Ph | N—Ph | b2 | H | H | a3 |
| 2-3-026 | N—Ph | N—Ph | b3 | H | H | a3 |
| 2-3-027 | N—Ph | N—Ph | H | H | H | a4 |
| 2-3-028 | N—Ph | N—Ph | t-bu | H | H | a4 |
| 2-3-029 | N—Ph | N—Ph | methyl | H | H | a4 |
| 2-3-030 | N—Ph | N—Ph | b1 | H | H | a4 |
| 2-3-031 | N—Ph | N—Ph | b2 | H | H | a4 |
| 2-3-032 | N—Ph | N—Ph | b3 | H | H | a4 |
| 2-3-033 | N—Ph | N—Ph | H | H | H | a5 |
| 2-3-034 | N—Ph | N—Ph | t-bu | H | H | a5 |
| 2-3-035 | N—Ph | N—Ph | methyl | H | H | a5 |
| 2-3-036 | N—Ph | N—Ph | b1 | H | H | a5 |
| 2-3-037 | N—Ph | N—Ph | b2 | H | H | a5 |
| 2-3-038 | N—Ph | N—Ph | b3 | H | H | a5 |
| 2-3-039 | N—Ph | N—Ph | H | H | H | a6 |
| 2-3-040 | N—Ph | N—Ph | t-bu | H | H | a6 |
| 2-3-041 | N—Ph | N—Ph | methyl | H | H | a6 |
| 2-3-042 | N—Ph | N—Ph | b1 | H | H | a6 |
| 2-3-043 | N—Ph | N—Ph | b2 | H | H | a6 |
| 2-3-044 | N—Ph | N—Ph | b3 | H | H | a6 |
| 2-3-045 | N—Ph | N—Ph | H | H | a1 | H |
| 2-3-046 | N—Ph | N—Ph | t-bu | H | a1 | H |
| 2-3-047 | N—Ph | N—Ph | methyl | H | a1 | H |
| 2-3-048 | N—Ph | N—Ph | b1 | H | a1 | H |
| 2-3-049 | N—Ph | N—Ph | b2 | H | a1 | H |
| 2-3-050 | N—Ph | N—Ph | b3 | H | a1 | H |
| 2-3-051 | N—Ph | N—Ph | H | H | a2 | H |
| 2-3-052 | N—Ph | N—Ph | t-bu | H | a2 | H |
| 2-3-053 | N—Ph | N—Ph | methyl | H | a2 | H |
| 2-3-054 | N—Ph | N—Ph | b1 | H | a2 | H |
| 2-3-055 | N—Ph | N—Ph | b2 | H | a2 | H |
| 2-3-056 | N—Ph | N—Ph | b3 | H | a2 | H |
| 2-3-057 | N—Ph | N—Ph | H | H | a3 | H |
| 2-3-058 | N—Ph | N—Ph | t-bu | H | a3 | H |
| 2-3-059 | N—Ph | N—Ph | methyl | H | a3 | H |
| 2-3-060 | N—Ph | N—Ph | b1 | H | a3 | H |
| 2-3-061 | N—Ph | N—Ph | b2 | H | a3 | H |
| 2-3-062 | N—Ph | N—Ph | b3 | H | a3 | H |
| 2-3-063 | N—Ph | N—Ph | H | H | a4 | H |
| 2-3-064 | N—Ph | N—Ph | t-bu | H | a4 | H |
| 2-3-065 | N—Ph | N—Ph | methyl | H | a4 | H |
| 2-3-066 | N—Ph | N—Ph | b1 | H | a4 | H |
| 2-3-067 | N—Ph | N—Ph | b2 | H | a4 | H |
| 2-3-068 | N—Ph | N—Ph | b3 | H | a4 | H |
| 2-3-069 | N—Ph | N—Ph | H | H | a5 | H |
| 2-3-070 | N—Ph | N—Ph | t-bu | H | a5 | H |
| 2-3-071 | N—Ph | N—Ph | methyl | H | a5 | H |
| 2-3-072 | N—Ph | N—Ph | b1 | H | a5 | H |
| 2-3-073 | N—Ph | N—Ph | b2 | H | a5 | H |
| 2-3-074 | N—Ph | N—Ph | b3 | H | a5 | H |
| 2-3-075 | N—Ph | N—Ph | H | H | a6 | H |
| 2-3-076 | N—Ph | N—Ph | t-bu | H | a6 | H |
| 2-3-077 | N—Ph | N—Ph | methyl | H | a6 | H |
| 2-3-078 | N—Ph | N—Ph | b1 | H | a6 | H |
| 2-3-079 | N—Ph | N—Ph | b2 | H | a6 | H |
| 2-3-080 | N—Ph | N—Ph | b3 | H | a6 | H |
| 2-3-081 | N—Ph | N—Ph | H | H | b1 | H |
| 2-3-082 | N—Ph | N—Ph | t-bu | H | b1 | H |
| 2-3-083 | N—Ph | N—Ph | methyl | H | b1 | H |
| 2-3-084 | N—Ph | N—Ph | b1 | H | b1 | H |
| 2-3-085 | N—Ph | N—Ph | b2 | H | b1 | H |
| 2-3-086 | N—Ph | N—Ph | b3 | H | b1 | H |
| 2-3-087 | N—Ph | N—Ph | H | H | b2 | H |
| 2-3-088 | N—Ph | N—Ph | t-bu | H | b2 | H |
| 2-3-089 | N—Ph | N—Ph | methyl | H | b2 | H |
| 2-3-090 | N—Ph | N—Ph | b1 | H | b2 | H |
| 2-3-091 | N—Ph | N—Ph | b2 | H | b2 | H |
| 2-3-092 | N—Ph | N—Ph | b3 | H | b2 | H |
| 2-3-093 | N—Ph | N—Ph | H | H | b3 | H |
| 2-3-094 | N—Ph | N—Ph | t-bu | H | b3 | H |
| 2-3-095 | N—Ph | N—Ph | methyl | H | b3 | H |
| 2-3-096 | N—Ph | N—Ph | b1 | H | b3 | H |
| 2-3-097 | N—Ph | N—Ph | b2 | H | b3 | H |
| 2-3-098 | N—Ph | N—Ph | b3 | H | b3 | H |
| 2-3-099 | N—Ph | N—Ph | H | H | b4 | H |
| 2-3-100 | N—Ph | N—Ph | t-bu | H | b4 | H |
| 2-3-101 | N—Ph | N—Ph | methyl | H | b4 | H |
| 2-3-102 | N—Ph | N—Ph | b1 | H | b4 | H |
| 2-3-103 | N—Ph | N—Ph | b2 | H | b4 | H |
| 2-3-104 | N—Ph | N—Ph | b3 | H | b4 | H |
| 2-3-105 | N—Ph | N—Ph | H | H | b5 | H |
| 2-3-106 | N—Ph | N—Ph | t-bu | H | b5 | H |
| 2-3-107 | N—Ph | N—Ph | methyl | H | b5 | H |
| 2-3-108 | N—Ph | N—Ph | b1 | H | b5 | H |
| 2-3-109 | N—Ph | N—Ph | b2 | H | b5 | H |
| 2-3-110 | N—Ph | N—Ph | b3 | H | b5 | H |
| 2-3-111 | N—Ph | N—Ph | H | H | b6 | H |
| 2-3-112 | N—Ph | N—Ph | t-bu | H | b6 | H |
| 2-3-113 | N—Ph | N—Ph | methyl | H | b6 | H |
| 2-3-114 | N—Ph | N—Ph | b1 | H | b6 | H |
| 2-3-115 | N—Ph | N—Ph | b2 | H | b6 | H |
| 2-3-116 | N—Ph | N—Ph | b3 | H | b6 | H |
| 2-3-117 | N—Ph | N—Ph | H | H | b7 | H |
| 2-3-118 | N—Ph | N—Ph | t-bu | H | b7 | H |
| 2-3-119 | N—Ph | N—Ph | methyl | H | b7 | H |
| 2-3-120 | N—Ph | N—Ph | b1 | H | b7 | H |
| 2-3-121 | N—Ph | N—Ph | b2 | H | b7 | H |
| 2-3-122 | N—Ph | N—Ph | b3 | H | b7 | H |
| 2-3-123 | N—Ph | N—Ph | H | H | b8 | H |
| 2-3-124 | N—Ph | N—Ph | t-bu | H | b8 | H |
| 2-3-125 | N—Ph | N—Ph | methyl | H | b8 | H |
| 2-3-126 | N—Ph | N—Ph | b1 | H | b8 | H |
| 2-3-127 | N—Ph | N—Ph | b2 | H | b8 | H |
| 2-3-128 | N—Ph | N—Ph | b3 | H | b8 | H |
| 2-3-129 | N—Ph | N—Ph | H | H | b9 | H |
| 2-3-130 | N—Ph | N—Ph | t-bu | H | b9 | H |
| 2-3-131 | N—Ph | N—Ph | methyl | H | b9 | H |
| 2-3-132 | N—Ph | N—Ph | b1 | H | b9 | H |
| 2-3-133 | N—Ph | N—Ph | b2 | H | b9 | H |
| 2-3-134 | N—Ph | N—Ph | b3 | H | b9 | H |
| 2-3-135 | N—Ph | N—Ph | H | H | b10 | H |
| 2-3-136 | N—Ph | N—Ph | t-bu | H | b10 | H |
| 2-3-137 | N—Ph | N—Ph | methyl | H | b10 | H |
| 2-3-138 | N—Ph | N—Ph | b1 | H | b10 | H |
| 2-3-139 | N—Ph | N—Ph | b2 | H | b10 | H |
| 2-3-140 | N—Ph | N—Ph | b3 | H | b10 | H |
| 2-3-141 | N—Ph | N—Ph | H | H | a1 | a1 |
| 2-3-142 | N—Ph | N—Ph | t-bu | H | a1 | a1 |
| 2-3-143 | N—Ph | N—Ph | methyl | H | a1 | a1 |
| 2-3-144 | N—Ph | N—Ph | b1 | H | a1 | a1 |
| 2-3-145 | N—Ph | N—Ph | b2 | H | a1 | a1 |
| 2-3-146 | N—Ph | N—Ph | b3 | H | a1 | a1 |
| 2-3-147 | N—Ph | N—Ph | H | H | a2 | a2 |
| 2-3-148 | N—Ph | N—Ph | t-bu | H | a2 | a2 |
| 2-3-149 | N—Ph | N—Ph | methyl | H | a2 | a2 |
| 2-3-150 | N—Ph | N—Ph | b1 | H | a2 | a2 |
| 2-3-151 | N—Ph | N—Ph | b2 | H | a2 | a2 |
| 2-3-152 | N—Ph | N—Ph | b3 | H | a2 | a2 |
| 2-3-153 | N—Ph | N—Ph | H | H | a3 | a3 |
| 2-3-154 | N—Ph | N—Ph | t-bu | H | a3 | a3 |
| 2-3-155 | N—Ph | N—Ph | methyl | H | a3 | a3 |
| 2-3-156 | N—Ph | N—Ph | b1 | H | a3 | a3 |
| 2-3-157 | N—Ph | N—Ph | b2 | H | a3 | a3 |
| 2-3-158 | N—Ph | N—Ph | b3 | H | a3 | a3 |
| 2-3-159 | N—Ph | N—Ph | H | H | b1 | b1 |
| 2-3-160 | N—Ph | N—Ph | t-bu | H | b1 | b1 |
| 2-3-161 | N—Ph | N—Ph | methyl | H | b1 | b1 |
| 2-3-162 | N—Ph | N—Ph | b1 | H | b1 | b1 |
| 2-3-163 | N—Ph | N—Ph | b2 | H | b1 | b1 |
| 2-3-164 | N—Ph | N—Ph | b3 | H | b1 | b1 |
| 2-3-165 | N—Ph | N—Ph | H | H | b2 | b2 |
| 2-3-166 | N—Ph | N—Ph | t-bu | H | b2 | b2 |
| 2-3-167 | N—Ph | N—Ph | methyl | H | b2 | b2 |
| 2-3-168 | N—Ph | N—Ph | b1 | H | b2 | b2 |
| 2-3-169 | N—Ph | N—Ph | b2 | H | b2 | b2 |
| 2-3-170 | N—Ph | N—Ph | b3 | H | b2 | b2 |
| 2-3-171 | N—Ph | N—Ph | H | H | b3 | b3 |
| 2-3-172 | N—Ph | N—Ph | t-bu | H | b3 | b3 |
| 2-3-173 | N—Ph | N—Ph | methyl | H | b3 | b3 |
| 2-3-174 | N—Ph | N—Ph | b1 | H | b3 | b3 |
| 2-3-175 | N—Ph | N—Ph | b2 | H | b3 | b3 |
| 2-3-176 | N—Ph | N—Ph | b3 | H | b3 | b3 |
| 2-3-177 | N—Ph | N—Ph | H | H | b5 | b5 |
| 2-3-178 | N—Ph | N—Ph | t-bu | H | b5 | b5 |
| 2-3-179 | N—Ph | N—Ph | methyl | H | b5 | b5 |
| 2-3-180 | N—Ph | N—Ph | b1 | H | b5 | b5 |
| 2-3-181 | N—Ph | N—Ph | b2 | H | b5 | b5 |
| 2-3-182 | N—Ph | N—Ph | b3 | H | b5 | b5 |
| 2-3-183 | N—Ph | N—Ph | H | H | b6 | b6 |
| 2-3-184 | N—Ph | N—Ph | t-bu | H | b6 | b6 |
| 2-3-185 | N—Ph | N—Ph | methyl | H | b6 | b6 |
| 2-3-186 | N—Ph | N—Ph | b1 | H | b6 | b6 |
| 2-3-187 | N—Ph | N—Ph | b2 | H | b6 | b6 |
| 2-3-188 | N—Ph | N—Ph | b3 | H | b6 | b6 |
| 2-3-189 | N—Ph | N—Ph | H | H | b7 | b7 |
| 2-3-190 | N—Ph | N—Ph | t-bu | H | b7 | b7 |
| 2-3-191 | N—Ph | N—Ph | methyl | H | b7 | b7 |
| 2-3-192 | N—Ph | N—Ph | b1 | H | b7 | b7 |
| 2-3-193 | N—Ph | N—Ph | b2 | H | b7 | b7 |
| 2-3-194 | N—Ph | N—Ph | b3 | H | b7 | b7 |
| 2-3-195 | N-tolyl | N-tolyl | H | H | H | H |
| 2-3-196 | N-tolyl | N-tolyl | t-bu | H | H | H |
| 2-3-197 | N-tolyl | N-tolyl | methyl | H | H | H |
| 2-3-198 | N-tolyl | N-tolyl | t-bu | t-bu | t-bu | t-bu |
| 2-3-199 | N-tolyl | N-tolyl | methyl | methyl | methyl | methyl |
| 2-3-200 | N-tolyl | N-tolyl | b1 | H | H | H |
| 2-3-201 | N-tolyl | N-tolyl | b2 | H | H | H |
| 2-3-202 | N-tolyl | N-tolyl | b3 | H | H | H |
| 2-3-203 | N-tolyl | N-tolyl | H | H | H | a1 |
| 2-3-204 | N-tolyl | N-tolyl | t-bu | H | H | a1 |
| 2-3-205 | N-tolyl | N-tolyl | methyl | H | H | a1 |
| 2-3-206 | N-tolyl | N-tolyl | b1 | H | H | a1 |
| 2-3-207 | N-tolyl | N-tolyl | b2 | H | H | a1 |
| 2-3-208 | N-tolyl | N-tolyl | b3 | H | H | a1 |
| 2-3-209 | N-tolyl | N-tolyl | H | H | H | a2 |
| 2-3-210 | N-tolyl | N-tolyl | t-bu | H | H | a2 |
| 2-3-211 | N-tolyl | N-tolyl | methyl | H | H | a2 |
| 2-3-212 | N-tolyl | N-tolyl | b1 | H | H | a2 |
| 2-3-213 | N-tolyl | N-tolyl | b2 | H | H | a2 |
| 2-3-214 | N-tolyl | N-tolyl | b3 | H | H | a2 |
| 2-3-215 | N-tolyl | N-tolyl | H | H | H | a3 |
| 2-3-216 | N-tolyl | N-tolyl | t-bu | H | H | a3 |
| 2-3-217 | N-tolyl | N-tolyl | methyl | H | H | a3 |
| 2-3-218 | N-tolyl | N-tolyl | b1 | H | H | a3 |
| 2-3-219 | N-tolyl | N-tolyl | b2 | H | H | a3 |
| 2-3-220 | N-tolyl | N-tolyl | b3 | H | H | a3 |
| 2-3-221 | N-tolyl | N-tolyl | H | H | H | a4 |
| 2-3-222 | N-tolyl | N-tolyl | t-bu | H | H | a4 |
| 2-3-223 | N-tolyl | N-tolyl | methyl | H | H | a4 |
| 2-3-224 | N-tolyl | N-tolyl | b1 | H | H | a4 |
| 2-3-225 | N-tolyl | N-tolyl | b2 | H | H | a4 |
| 2-3-226 | N-tolyl | N-tolyl | b3 | H | H | a4 |
| 2-3-227 | N-tolyl | N-tolyl | H | H | H | a5 |
| 2-3-228 | N-tolyl | N-tolyl | t-bu | H | H | a5 |
| 2-3-229 | N-tolyl | N-tolyl | methyl | H | H | a5 |
| 2-3-230 | N-tolyl | N-tolyl | b1 | H | H | a5 |
| 2-3-231 | N-tolyl | N-tolyl | b2 | H | H | a5 |
| 2-3-232 | N-tolyl | N-tolyl | b3 | H | H | a5 |
| 2-3-233 | N-tolyl | N-tolyl | H | H | H | a6 |
| 2-3-234 | N-tolyl | N-tolyl | t-bu | H | H | a6 |
| 2-3-235 | N-tolyl | N-tolyl | methyl | H | H | a6 |
| 2-3-236 | N-tolyl | N-tolyl | b1 | H | H | a6 |
| 2-3-237 | N-tolyl | N-tolyl | b2 | H | H | a6 |
| 2-3-238 | N-tolyl | N-tolyl | b3 | H | H | a6 |
| 2-3-239 | N-tolyl | N-tolyl | H | H | a1 | H |
| 2-3-240 | N-tolyl | N-tolyl | t-bu | H | a1 | H |
| 2-3-241 | N-tolyl | N-tolyl | methyl | H | a1 | H |
| 2-3-242 | N-tolyl | N-tolyl | b1 | H | a1 | H |
| 2-3-243 | N-tolyl | N-tolyl | b2 | H | a1 | H |
| 2-3-244 | N-tolyl | N-tolyl | b3 | H | a1 | H |
| 2-3-245 | N-tolyl | N-tolyl | H | H | a2 | H |
| 2-3-246 | N-tolyl | N-tolyl | t-bu | H | a2 | H |
| 2-3-247 | N-tolyl | N-tolyl | methyl | H | a2 | H |
| 2-3-248 | N-tolyl | N-tolyl | b1 | H | a2 | H |
| 2-3-249 | N-tolyl | N-tolyl | b2 | H | a2 | H |
| 2-3-250 | N-tolyl | N-tolyl | b3 | H | a2 | H |
| 2-3-251 | N-tolyl | N-tolyl | H | H | a3 | H |
| 2-3-252 | N-tolyl | N-tolyl | t-bu | H | a3 | H |
| 2-3-253 | N-tolyl | N-tolyl | methyl | H | a3 | H |
| 2-3-254 | N-tolyl | N-tolyl | b1 | H | a3 | H |
| 2-3-255 | N-tolyl | N-tolyl | b2 | H | a3 | H |
| 2-3-256 | N-tolyl | N-tolyl | b3 | H | a3 | H |
| 2-3-257 | N-tolyl | N-tolyl | H | H | a4 | H |
| 2-3-258 | N-tolyl | N-tolyl | t-bu | H | a4 | H |
| 2-3-259 | N-tolyl | N-tolyl | methyl | H | a4 | H |
| 2-3-260 | N-tolyl | N-tolyl | b1 | H | a4 | H |
| 2-3-261 | N-tolyl | N-tolyl | b2 | H | a4 | H |
| 2-3-262 | N-tolyl | N-tolyl | b3 | H | a4 | H |
| 2-3-263 | N-tolyl | N-tolyl | H | H | a5 | H |
| 2-3-264 | N-tolyl | N-tolyl | t-bu | H | a5 | H |
| 2-3-265 | N-tolyl | N-tolyl | methyl | H | a5 | H |
| 2-3-266 | N-tolyl | N-tolyl | b1 | H | a5 | H |
| 2-3-267 | N-tolyl | N-tolyl | b2 | H | a5 | H |
| 2-3-268 | N-tolyl | N-tolyl | b3 | H | a5 | H |
| 2-3-269 | N-tolyl | N-tolyl | H | H | a6 | H |
| 2-3-270 | N-tolyl | N-tolyl | t-bu | H | a6 | H |
| 2-3-271 | N-tolyl | N-tolyl | methyl | H | a6 | H |
| 2-3-272 | N-tolyl | N-tolyl | b1 | H | a6 | H |
| 2-3-273 | N-tolyl | N-tolyl | b2 | H | a6 | H |
| 2-3-274 | N-tolyl | N-tolyl | b3 | H | a6 | H |
| 2-3-275 | N-tolyl | N-tolyl | H | H | b1 | H |
| 2-3-276 | N-tolyl | N-tolyl | t-bu | H | b1 | H |
| 2-3-277 | N-tolyl | N-tolyl | methyl | H | b1 | H |
| 2-3-278 | N-tolyl | N-tolyl | b1 | H | b1 | H |
| 2-3-279 | N-tolyl | N-tolyl | b2 | H | b1 | H |
| 2-3-280 | N-tolyl | N-tolyl | b3 | H | b1 | H |
| 2-3-281 | N-tolyl | N-tolyl | H | H | b2 | H |
| 2-3-282 | N-tolyl | N-tolyl | t-bu | H | b2 | H |
| 2-3-283 | N-tolyl | N-tolyl | methyl | H | b2 | H |
| 2-3-284 | N-tolyl | N-tolyl | b1 | H | b2 | H |
| 2-3-285 | N-tolyl | N-tolyl | b2 | H | b2 | H |
| 2-3-286 | N-tolyl | N-tolyl | b3 | H | b2 | H |
| 2-3-287 | N-tolyl | N-tolyl | H | H | b3 | H |
| 2-3-288 | N-tolyl | N-tolyl | t-bu | H | b3 | H |
| 2-3-289 | N-tolyl | N-tolyl | methyl | H | b3 | H |
| 2-3-290 | N-tolyl | N-tolyl | b1 | H | b3 | H |
| 2-3-291 | N-tolyl | N-tolyl | b2 | H | b3 | H |
| 2-3-292 | N-tolyl | N-tolyl | b3 | H | b3 | H |
| 2-3-293 | N-tolyl | N-tolyl | H | H | b4 | H |
| 2-3-294 | N-tolyl | N-tolyl | t-bu | H | b4 | H |
| 2-3-295 | N-tolyl | N-tolyl | methyl | H | b4 | H |
| 2-3-296 | N-tolyl | N-tolyl | b1 | H | b4 | H |
| 2-3-297 | N-tolyl | N-tolyl | b2 | H | b4 | H |
| 2-3-298 | N-tolyl | N-tolyl | b3 | H | b4 | H |
| 2-3-299 | N-tolyl | N-tolyl | H | H | b5 | H |
| 2-3-300 | N-tolyl | N-tolyl | t-bu | H | b5 | H |
| 2-3-301 | N-tolyl | N-tolyl | methyl | H | b5 | H |
| 2-3-302 | N-tolyl | N-tolyl | b1 | H | b5 | H |
| 2-3-303 | N-tolyl | N-tolyl | b2 | H | b5 | H |
| 2-3-304 | N-tolyl | N-tolyl | b3 | H | b5 | H |
| 2-3-305 | N-tolyl | N-tolyl | H | H | b6 | H |
| 2-3-306 | N-tolyl | N-tolyl | t-bu | H | b6 | H |
| 2-3-307 | N-tolyl | N-tolyl | methyl | H | b6 | H |
| 2-3-308 | N-tolyl | N-tolyl | b1 | H | b6 | H |
| 2-3-309 | N-tolyl | N-tolyl | b2 | H | b6 | H |
| 2-3-310 | N-tolyl | N-tolyl | b3 | H | b6 | H |
| 2-3-311 | N-tolyl | N-tolyl | H | H | b7 | H |
| 2-3-312 | N-tolyl | N-tolyl | t-bu | H | b7 | H |
| 2-3-313 | N-tolyl | N-tolyl | methyl | H | b7 | H |
| 2-3-314 | N-tolyl | N-tolyl | b1 | H | b7 | H |
| 2-3-315 | N-tolyl | N-tolyl | b2 | H | b7 | H |
| 2-3-316 | N-tolyl | N-tolyl | b3 | H | b7 | H |
| 2-3-317 | N-tolyl | N-tolyl | H | H | b8 | H |
| 2-3-318 | N-tolyl | N-tolyl | t-bu | H | b8 | H |
| 2-3-319 | N-tolyl | N-tolyl | methyl | H | b8 | H |
| 2-3-320 | N-tolyl | N-tolyl | b1 | H | b8 | H |
| 2-3-321 | N-tolyl | N-tolyl | b2 | H | b8 | H |
| 2-3-322 | N-tolyl | N-tolyl | b3 | H | b8 | H |
| 2-3-323 | N-tolyl | N-tolyl | H | H | b9 | H |
| 2-3-324 | N-tolyl | N-tolyl | t-bu | H | b9 | H |
| 2-3-325 | N-tolyl | N-tolyl | methyl | H | b9 | H |
| 2-3-326 | N-tolyl | N-tolyl | b1 | H | b9 | H |
| 2-3-327 | N-tolyl | N-tolyl | b2 | H | b9 | H |
| 2-3-328 | N-tolyl | N-tolyl | b3 | H | b9 | H |
| 2-3-329 | N-tolyl | N-tolyl | H | H | b10 | H |
| 2-3-330 | N-tolyl | N-tolyl | t-bu | H | b10 | H |
| 2-3-331 | N-tolyl | N-tolyl | methyl | H | b10 | H |
| 2-3-332 | N-tolyl | N-tolyl | b1 | H | b10 | H |
| 2-3-333 | N-tolyl | N-tolyl | b2 | H | b10 | H |
| 2-3-334 | N-tolyl | N-tolyl | b3 | H | b10 | H |
| 2-3-335 | N-tolyl | N-tolyl | H | H | a1 | a1 |
| 2-3-336 | N-tolyl | N-tolyl | t-bu | H | a1 | a1 |
| 2-3-337 | N-tolyl | N-tolyl | methyl | H | a1 | a1 |
| 2-3-338 | N-tolyl | N-tolyl | b1 | H | a1 | a1 |
| 2-3-339 | N-tolyl | N-tolyl | b2 | H | a1 | a1 |
| 2-3-340 | N-tolyl | N-tolyl | b3 | H | a1 | a1 |
| 2-3-341 | N-tolyl | N-tolyl | H | H | a2 | a2 |
| 2-3-342 | N-tolyl | N-tolyl | t-bu | H | a2 | a2 |
| 2-3-343 | N-tolyl | N-tolyl | methyl | H | a2 | a2 |
| 2-3-344 | N-tolyl | N-tolyl | b1 | H | a2 | a2 |
| 2-3-345 | N-tolyl | N-tolyl | b2 | H | a2 | a2 |
| 2-3-346 | N-tolyl | N-tolyl | b3 | H | a2 | a2 |
| 2-3-347 | N-tolyl | N-tolyl | H | H | a3 | a3 |
| 2-3-348 | N-tolyl | N-tolyl | t-bu | H | a3 | a3 |
| 2-3-349 | N-tolyl | N-tolyl | methyl | H | a3 | a3 |
| 2-3-350 | N-tolyl | N-tolyl | b1 | H | a3 | a3 |
| 2-3-351 | N-tolyl | N-tolyl | b2 | H | a3 | a3 |
| 2-3-352 | N-tolyl | N-tolyl | b3 | H | a3 | a3 |
| 2-3-353 | N-tolyl | N-tolyl | H | H | b1 | b1 |
| 2-3-354 | N-tolyl | N-tolyl | t-bu | H | b1 | b1 |
| 2-3-355 | N-tolyl | N-tolyl | methyl | H | b1 | b1 |
| 2-3-356 | N-tolyl | N-tolyl | b1 | H | b1 | b1 |
| 2-3-357 | N-tolyl | N-tolyl | b2 | H | b1 | b1 |
| 2-3-358 | N-tolyl | N-tolyl | b3 | H | b1 | b1 |
| 2-3-359 | N-tolyl | N-tolyl | H | H | b2 | b2 |
| 2-3-360 | N-tolyl | N-tolyl | t-bu | H | b2 | b2 |
| 2-3-361 | N-tolyl | N-tolyl | methyl | H | b2 | b2 |
| 2-3-362 | N-tolyl | N-tolyl | b1 | H | b2 | b2 |
| 2-3-363 | N-tolyl | N-tolyl | b2 | H | b2 | b2 |
| 2-3-364 | N-tolyl | N-tolyl | b3 | H | b2 | b2 |
| 2-3-365 | N-tolyl | N-tolyl | H | H | b3 | b3 |
| 2-3-366 | N-tolyl | N-tolyl | t-bu | H | b3 | b3 |
| 2-3-367 | N-tolyl | N-tolyl | methyl | H | b3 | b3 |
| 2-3-368 | N-tolyl | N-tolyl | b1 | H | b3 | b3 |
| 2-3-369 | N-tolyl | N-tolyl | b2 | H | b3 | b3 |
| 2-3-370 | N-tolyl | N-tolyl | b3 | H | b3 | b3 |
| 2-3-371 | N-tolyl | N-tolyl | H | H | b5 | b5 |
| 2-3-372 | N-tolyl | N-tolyl | t-bu | H | b5 | b5 |
| 2-3-373 | N-tolyl | N-tolyl | methyl | H | b5 | b5 |
| 2-3-374 | N-tolyl | N-tolyl | b1 | H | b5 | b5 |
| 2-3-375 | N-tolyl | N-tolyl | b2 | H | b5 | b5 |
| 2-3-376 | N-tolyl | N-tolyl | b3 | H | b5 | b5 |
| 2-3-377 | N-tolyl | N-tolyl | H | H | b6 | b6 |
| 2-3-378 | N-tolyl | N-tolyl | t-bu | H | b6 | b6 |
| 2-3-379 | N-tolyl | N-tolyl | methyl | H | b6 | b6 |
| 2-3-380 | N-tolyl | N-tolyl | b1 | H | b6 | b6 |
| 2-3-381 | N-tolyl | N-tolyl | b2 | H | b6 | b6 |
| 2-3-382 | N-tolyl | N-tolyl | b3 | H | b6 | b6 |
| 2-3-383 | N-tolyl | N-tolyl | H | H | b7 | b7 |
| 2-3-384 | N-tolyl | N-tolyl | t-bu | H | b7 | b7 |
| 2-3-385 | N-tolyl | N-tolyl | methyl | H | b7 | b7 |
| 2-3-386 | N-tolyl | N-tolyl | b1 | H | b7 | b7 |
| 2-3-387 | N-tolyl | N-tolyl | b2 | H | b7 | b7 |
| 2-3-388 | N-tolyl | N-tolyl | b3 | H | b7 | b7 |
| 2-3-389 | N-Biph | N-Biph | H | H | H | H |
| 2-3-390 | N-Biph | N-Biph | t-bu | H | H | H |
| 2-3-391 | N-Biph | N-Biph | methyl | H | H | H |
| 2-3-392 | N-Biph | N-Biph | t-bu | t-bu | t-bu | t-bu |
| 2-3-393 | N-Biph | N-Biph | methyl | methyl | methyl | methyl |
| 2-3-394 | N-Biph | N-Biph | b1 | H | H | H |
| 2-3-395 | N-Biph | N-Biph | b2 | H | H | H |
| 2-3-396 | N-Biph | N-Biph | b3 | H | H | H |
| 2-3-397 | N-Biph | N-Biph | H | H | H | a1 |
| 2-3-398 | N-Biph | N-Biph | t-bu | H | H | a1 |
| 2-3-399 | N-Biph | N-Biph | methyl | H | H | a1 |
| 2-3-400 | N-Biph | N-Biph | b1 | H | H | a1 |
| 2-3-401 | N-Biph | N-Biph | b2 | H | H | a1 |
| 2-3-402 | N-Biph | N-Biph | b3 | H | H | a1 |
| 2-3-403 | N-Biph | N-Biph | H | H | H | a2 |
| 2-3-404 | N-Biph | N-Biph | t-bu | H | H | a2 |
| 2-3-405 | N-Biph | N-Biph | methyl | H | H | a2 |
| 2-3-406 | N-Biph | N-Biph | b1 | H | H | a2 |
| 2-3-407 | N-Biph | N-Biph | b2 | H | H | a2 |
| 2-3-408 | N-Biph | N-Biph | b3 | H | H | a2 |
| 2-3-409 | N-Biph | N-Biph | H | H | H | a3 |
| 2-3-410 | N-Biph | N-Biph | t-bu | H | H | a3 |
| 2-3-411 | N-Biph | N-Biph | methyl | H | H | a3 |
| 2-3-412 | N-Biph | N-Biph | b1 | H | H | a3 |
| 2-3-413 | N-Biph | N-Biph | b2 | H | H | a3 |
| 2-3-414 | N-Biph | N-Biph | b3 | H | H | a3 |
| 2-3-415 | N-Biph | N-Biph | H | H | H | a4 |
| 2-3-416 | N-Biph | N-Biph | t-bu | H | H | a4 |
| 2-3-417 | N-Biph | N-Biph | methyl | H | H | a4 |
| 2-3-418 | N-Biph | N-Biph | b1 | H | H | a4 |
| 2-3-419 | N-Biph | N-Biph | b2 | H | H | a4 |
| 2-3-420 | N-Biph | N-Biph | b3 | H | H | a4 |
| 2-3-421 | N-Biph | N-Biph | H | H | H | a5 |
| 2-3-422 | N-Biph | N-Biph | t-bu | H | H | a5 |
| 2-3-423 | N-Biph | N-Biph | methyl | H | H | a5 |
| 2-3-424 | N-Biph | N-Biph | b1 | H | H | a5 |
| 2-3-425 | N-Biph | N-Biph | b2 | H | H | a5 |
| 2-3-426 | N-Biph | N-Biph | b3 | H | H | a5 |
| 2-3-427 | N-Biph | N-Biph | H | H | H | a6 |
| 2-3-428 | N-Biph | N-Biph | t-bu | H | H | a6 |
| 2-3-429 | N-Biph | N-Biph | methyl | H | H | a6 |
| 2-3-430 | N-Biph | N-Biph | b1 | H | H | a6 |
| 2-3-431 | N-Biph | N-Biph | b2 | H | H | a6 |
| 2-3-432 | N-Biph | N-Biph | b3 | H | H | a6 |
| 2-3-433 | N-Biph | N-Biph | H | H | a1 | H |
| 2-3-434 | N-Biph | N-Biph | t-bu | H | a1 | H |
| 2-3-435 | N-Biph | N-Biph | methyl | H | a1 | H |
| 2-3-436 | N-Biph | N-Biph | b1 | H | a1 | H |
| 2-3-437 | N-Biph | N-Biph | b2 | H | a1 | H |
| 2-3-438 | N-Biph | N-Biph | b3 | H | a1 | H |
| 2-3-439 | N-Biph | N-Biph | H | H | a2 | H |
| 2-3-440 | N-Biph | N-Biph | t-bu | H | a2 | H |
| 2-3-441 | N-Biph | N-Biph | methyl | H | a2 | H |
| 2-3-442 | N-Biph | N-Biph | b1 | H | a2 | H |
| 2-3-443 | N-Biph | N-Biph | b2 | H | a2 | H |
| 2-3-444 | N-Biph | N-Biph | b3 | H | a2 | H |
| 2-3-445 | N-Biph | N-Biph | H | H | a3 | H |
| 2-3-446 | N-Biph | N-Biph | t-bu | H | a3 | H |
| 2-3-447 | N-Biph | N-Biph | methyl | H | a3 | H |
| 2-3-448 | N-Biph | N-Biph | b1 | H | a3 | H |
| 2-3-449 | N-Biph | N-Biph | b2 | H | a3 | H |
| 2-3-450 | N-Biph | N-Biph | b3 | H | a3 | H |
| 2-3-451 | N-Biph | N-Biph | H | H | a4 | H |
| 2-3-452 | N-Biph | N-Biph | t-bu | H | a4 | H |
| 2-3-453 | N-Biph | N-Biph | methyl | H | a4 | H |
| 2-3-454 | N-Biph | N-Biph | b1 | H | a4 | H |
| 2-3-455 | N-Biph | N-Biph | b2 | H | a4 | H |
| 2-3-456 | N-Biph | N-Biph | b3 | H | a4 | H |
| 2-3-457 | N-Biph | N-Biph | H | H | a5 | H |
| 2-3-458 | N-Biph | N-Biph | t-bu | H | a5 | H |
| 2-3-459 | N-Biph | N-Biph | methyl | H | a5 | H |
| 2-3-460 | N-Biph | N-Biph | b1 | H | a5 | H |
| 2-3-461 | N-Biph | N-Biph | b2 | H | a5 | H |
| 2-3-462 | N-Biph | N-Biph | b3 | H | a5 | H |
| 2-3-463 | N-Biph | N-Biph | H | H | a6 | H |
| 2-3-464 | N-Biph | N-Biph | t-bu | H | a6 | H |
| 2-3-465 | N-Biph | N-Biph | methyl | H | a6 | H |
| 2-3-466 | N-Biph | N-Biph | b1 | H | a6 | H |
| 2-3-467 | N-Biph | N-Biph | b2 | H | a6 | H |
| 2-3-468 | N-Biph | N-Biph | b3 | H | a6 | H |
| 2-3-469 | N-Biph | N-Biph | H | H | b1 | H |
| 2-3-470 | N-Biph | N-Biph | t-bu | H | b1 | H |
| 2-3-471 | N-Biph | N-Biph | methyl | H | b1 | H |
| 2-3-472 | N-Biph | N-Biph | b1 | H | b1 | H |
| 2-3-473 | N-Biph | N-Biph | b2 | H | b1 | H |
| 2-3-474 | N-Biph | N-Biph | b3 | H | b1 | H |
| 2-3-475 | N-Biph | N-Biph | H | H | b2 | H |
| 2-3-476 | N-Biph | N-Biph | t-bu | H | b2 | H |
| 2-3-477 | N-Biph | N-Biph | methyl | H | b2 | H |
| 2-3-478 | N-Biph | N-Biph | b1 | H | b2 | H |
| 2-3-479 | N-Biph | N-Biph | b2 | H | b2 | H |
| 2-3-480 | N-Biph | N-Biph | b3 | H | b2 | H |
| 2-3-481 | N-Biph | N-Biph | H | H | b3 | H |
| 2-3-482 | N-Biph | N-Biph | t-bu | H | b3 | H |
| 2-3-483 | N-Biph | N-Biph | methyl | H | b3 | H |
| 2-3-484 | N-Biph | N-Biph | b1 | H | b3 | H |
| 2-3-485 | N-Biph | N-Biph | b2 | H | b3 | H |
| 2-3-486 | N-Biph | N-Biph | b3 | H | b3 | H |
| 2-3-487 | N-Biph | N-Biph | H | H | b4 | H |
| 2-3-488 | N-Biph | N-Biph | t-bu | H | b4 | H |
| 2-3-489 | N-Biph | N-Biph | methyl | H | b4 | H |
| 2-3-490 | N-Biph | N-Biph | b1 | H | b4 | H |
| 2-3-491 | N-Biph | N-Biph | b2 | H | b4 | H |
| 2-3-492 | N-Biph | N-Biph | b3 | H | b4 | H |
| 2-3-493 | N-Biph | N-Biph | H | H | b5 | H |
| 2-3-494 | N-Biph | N-Biph | t-bu | H | b5 | H |
| 2-3-495 | N-Biph | N-Biph | methyl | H | b5 | H |
| 2-3-496 | N-Biph | N-Biph | b1 | H | b5 | H |
| 2-3-497 | N-Biph | N-Biph | b2 | H | b5 | H |
| 2-3-498 | N-Biph | N-Biph | b3 | H | b5 | H |
| 2-3-499 | N-Biph | N-Biph | H | H | b6 | H |
| 2-3-500 | N-Biph | N-Biph | t-bu | H | b6 | H |
| 2-3-501 | N-Biph | N-Biph | methyl | H | b6 | H |
| 2-3-502 | N-Biph | N-Biph | b1 | H | b6 | H |
| 2-3-503 | N-Biph | N-Biph | b2 | H | b6 | H |
| 2-3-504 | N-Biph | N-Biph | b3 | H | b6 | H |
| 2-3-505 | N-Biph | N-Biph | H | H | b7 | H |
| 2-3-506 | N-Biph | N-Biph | t-bu | H | b7 | H |
| 2-3-507 | N-Biph | N-Biph | methyl | H | b7 | H |
| 2-3-508 | N-Biph | N-Biph | b1 | H | b7 | H |
| 2-3-509 | N-Biph | N-Biph | b2 | H | b7 | H |
| 2-3-510 | N-Biph | N-Biph | b3 | H | b7 | H |
| 2-3-511 | N-Biph | N-Biph | H | H | b8 | H |
| 2-3-512 | N-Biph | N-Biph | t-bu | H | b8 | H |
| 2-3-513 | N-Biph | N-Biph | methyl | H | b8 | H |
| 2-3-514 | N-Biph | N-Biph | b1 | H | b8 | H |
| 2-3-515 | N-Biph | N-Biph | b2 | H | b8 | H |
| 2-3-516 | N-Biph | N-Biph | b3 | H | b8 | H |
| 2-3-517 | N-Biph | N-Biph | H | H | b9 | H |
| 2-3-518 | N-Biph | N-Biph | t-bu | H | b9 | H |
| 2-3-519 | N-Biph | N-Biph | methyl | H | b9 | H |
| 2-3-520 | N-Biph | N-Biph | b1 | H | b9 | H |
| 2-3-521 | N-Biph | N-Biph | b2 | H | b9 | H |
| 2-3-522 | N-Biph | N-Biph | b3 | H | b9 | H |
| 2-3-523 | N-Biph | N-Biph | H | H | b10 | H |
| 2-3-524 | N-Biph | N-Biph | t-bu | H | b10 | H |
| 2-3-525 | N-Biph | N-Biph | methyl | H | b10 | H |
| 2-3-526 | N-Biph | N-Biph | b1 | H | b10 | H |
| 2-3-527 | N-Biph | N-Biph | b2 | H | b10 | H |
| 2-3-528 | N-Biph | N-Biph | b3 | H | b10 | H |
| 2-3-529 | N-Biph | N-Biph | H | H | a1 | a1 |
| 2-3-530 | N-Biph | N-Biph | t-bu | H | a1 | a1 |
| 2-3-531 | N-Biph | N-Biph | methyl | H | a1 | a1 |
| 2-3-532 | N-Biph | N-Biph | b1 | H | a1 | a1 |
| 2-3-533 | N-Biph | N-Biph | b2 | H | a1 | a1 |
| 2-3-534 | N-Biph | N-Biph | b3 | H | a1 | a1 |
| 2-3-535 | N-Biph | N-Biph | H | H | a2 | a2 |
| 2-3-536 | N-Biph | N-Biph | t-bu | H | a2 | a2 |
| 2-3-537 | N-Biph | N-Biph | methyl | H | a2 | a2 |
| 2-3-538 | N-Biph | N-Biph | b1 | H | a2 | a2 |
| 2-3-539 | N-Biph | N-Biph | b2 | H | a2 | a2 |
| 2-3-540 | N-Biph | N-Biph | b3 | H | a2 | a2 |
| 2-3-541 | N-Biph | N-Biph | H | H | a3 | a3 |
| 2-3-542 | N-Biph | N-Biph | t-bu | H | a3 | a3 |
| 2-3-543 | N-Biph | N-Biph | methyl | H | a3 | a3 |
| 2-3-544 | N-Biph | N-Biph | b1 | H | a3 | a3 |
| 2-3-545 | N-Biph | N-Biph | b2 | H | a3 | a3 |
| 2-3-546 | N-Biph | N-Biph | b3 | H | a3 | a3 |
| 2-3-547 | N-Biph | N-Biph | H | H | b1 | b1 |
| 2-3-548 | N-Biph | N-Biph | t-bu | H | b1 | b1 |
| 2-3-549 | N-Biph | N-Biph | methyl | H | b1 | b1 |
| 2-3-550 | N-Biph | N-Biph | b1 | H | b1 | b1 |
| 2-3-551 | N-Biph | N-Biph | b2 | H | b1 | b1 |
| 2-3-552 | N-Biph | N-Biph | b3 | H | b1 | b1 |
| 2-3-553 | N-Biph | N-Biph | H | H | b2 | b2 |
| 2-3-554 | N-Biph | N-Biph | t-bu | H | b2 | b2 |
| 2-3-555 | N-Biph | N-Biph | methyl | H | b2 | b2 |
| 2-3-556 | N-Biph | N-Biph | b1 | H | b2 | b2 |
| 2-3-557 | N-Biph | N-Biph | b2 | H | b2 | b2 |
| 2-3-558 | N-Biph | N-Biph | b3 | H | b2 | b2 |
| 2-3-559 | N-Biph | N-Biph | H | H | b3 | b3 |
| 2-3-560 | N-Biph | N-Biph | t-bu | H | b3 | b3 |
| 2-3-561 | N-Biph | N-Biph | methyl | H | b3 | b3 |
| 2-3-562 | N-Biph | N-Biph | b1 | H | b3 | b3 |
| 2-3-563 | N-Biph | N-Biph | b2 | H | b3 | b3 |
| 2-3-564 | N-Biph | N-Biph | b3 | H | b3 | b3 |
| 2-3-565 | N-Biph | N-Biph | H | H | b5 | b5 |
| 2-3-566 | N-Biph | N-Biph | t-bu | H | b5 | b5 |
| 2-3-567 | N-Biph | N-Biph | methyl | H | b5 | b5 |
| 2-3-568 | N-Biph | N-Biph | b1 | H | b5 | b5 |
| 2-3-569 | N-Biph | N-Biph | b2 | H | b5 | b5 |
| 2-3-570 | N-Biph | N-Biph | b3 | H | b5 | b5 |
| 2-3-571 | N-Biph | N-Biph | H | H | b6 | b6 |
| 2-3-572 | N-Biph | N-Biph | t-bu | H | b6 | b6 |
| 2-3-573 | N-Biph | N-Biph | methyl | H | b6 | b6 |
| 2-3-574 | N-Biph | N-Biph | b1 | H | b6 | b6 |
| 2-3-575 | N-Biph | N-Biph | b2 | H | b6 | b6 |
| 2-3-576 | N-Biph | N-Biph | b3 | H | b6 | b6 |
| 2-3-577 | N-Biph | N-Biph | H | H | b7 | b7 |
| 2-3-578 | N-Biph | N-Biph | t-bu | H | b7 | b7 |
| 2-3-579 | N-Biph | N-Biph | methyl | H | b7 | b7 |
| 2-3-580 | N-Biph | N-Biph | b1 | H | b7 | b7 |
| 2-3-581 | N-Biph | N-Biph | b2 | H | b7 | b7 |
| 2-3-582 | N-Biph | N-Biph | b3 | H | b7 | b7 |
| 2-3-583 | O | N—Ph | H | H | H | H |
| 2-3-584 | O | N—Ph | t-bu | H | H | H |
| 2-3-585 | O | N—Ph | methyl | H | H | H |
| 2-3-586 | O | N—Ph | t-bu | t-bu | t-bu | t-bu |
| 2-3-587 | O | N—Ph | methyl | methyl | methyl | methyl |
| 2-3-588 | O | N—Ph | b1 | H | H | H |
| 2-3-589 | O | N—Ph | b2 | H | H | H |
| 2-3-590 | O | N—Ph | b3 | H | H | H |
| 2-3-591 | O | N—Ph | H | H | H | a1 |
| 2-3-592 | O | N—Ph | t-bu | H | H | a1 |
| 2-3-593 | O | N—Ph | methyl | H | H | a1 |
| 2-3-594 | O | N—Ph | b1 | H | H | a1 |
| 2-3-595 | O | N—Ph | b2 | H | H | a1 |
| 2-3-596 | O | N—Ph | b3 | H | H | a1 |
| 2-3-597 | O | N—Ph | H | H | H | a2 |
| 2-3-598 | O | N—Ph | t-bu | H | H | a2 |
| 2-3-599 | O | N—Ph | methyl | H | H | a2 |
| 2-3-600 | O | N—Ph | b1 | H | H | a2 |
| 2-3-601 | O | N—Ph | b2 | H | H | a2 |
| 2-3-602 | O | N—Ph | b3 | H | H | a2 |
| 2-3-603 | O | N—Ph | H | H | H | a3 |
| 2-3-604 | O | N—Ph | t-bu | H | H | a3 |
| 2-3-605 | O | N—Ph | methyl | H | H | a3 |
| 2-3-606 | O | N—Ph | b1 | H | H | a3 |
| 2-3-607 | O | N—Ph | b2 | H | H | a3 |
| 2-3-608 | O | N—Ph | b3 | H | H | a3 |
| 2-3-609 | O | N—Ph | H | H | H | a4 |
| 2-3-610 | O | N—Ph | t-bu | H | H | a4 |
| 2-3-611 | O | N—Ph | methyl | H | H | a4 |
| 2-3-612 | O | N—Ph | b1 | H | H | a4 |
| 2-3-613 | O | N—Ph | b2 | H | H | a4 |
| 2-3-614 | O | N—Ph | b3 | H | H | a4 |
| 2-3-615 | O | N—Ph | H | H | H | a5 |
| 2-3-616 | O | N—Ph | t-bu | H | H | a5 |
| 2-3-617 | O | N—Ph | methyl | H | H | a5 |
| 2-3-618 | O | N—Ph | b1 | H | H | a5 |
| 2-3-619 | O | N—Ph | b2 | H | H | a5 |
| 2-3-620 | O | N—Ph | b3 | H | H | a5 |
| 2-3-621 | O | N—Ph | H | H | H | a6 |
| 2-3-622 | O | N—Ph | t-bu | H | H | a6 |
| 2-3-623 | O | N—Ph | methyl | H | H | a6 |
| 2-3-624 | O | N—Ph | b1 | H | H | a6 |
| 2-3-625 | O | N—Ph | b2 | H | H | a6 |
| 2-3-626 | O | N—Ph | b3 | H | H | a6 |
| 2-3-627 | O | N—Ph | H | H | a1 | H |
| 2-3-628 | O | N—Ph | t-bu | H | a1 | H |
| 2-3-629 | O | N—Ph | methyl | H | a1 | H |
| 2-3-630 | O | N—Ph | b1 | H | a1 | H |
| 2-3-631 | O | N—Ph | b2 | H | a1 | H |
| 2-3-632 | O | N—Ph | b3 | H | a1 | H |
| 2-3-633 | O | N—Ph | H | H | a2 | H |
| 2-3-634 | O | N—Ph | t-bu | H | a2 | H |
| 2-3-635 | O | N—Ph | methyl | H | a2 | H |
| 2-3-636 | O | N—Ph | b1 | H | a2 | H |
| 2-3-637 | O | N—Ph | b2 | H | a2 | H |
| 2-3-638 | O | N—Ph | b3 | H | a2 | H |
| 2-3-639 | O | N—Ph | H | H | a3 | H |
| 2-3-640 | O | N—Ph | t-bu | H | a3 | H |
| 2-3-641 | O | N—Ph | methyl | H | a3 | H |
| 2-3-642 | O | N—Ph | b1 | H | a3 | H |
| 2-3-643 | O | N—Ph | b2 | H | a3 | H |
| 2-3-644 | O | N—Ph | b3 | H | a3 | H |
| 2-3-645 | O | N—Ph | H | H | a4 | H |
| 2-3-646 | O | N—Ph | t-bu | H | a4 | H |
| 2-3-647 | O | N—Ph | methyl | H | a4 | H |
| 2-3-648 | O | N—Ph | b1 | H | a4 | H |
| 2-3-649 | O | N—Ph | b2 | H | a4 | H |
| 2-3-650 | O | N—Ph | b3 | H | a4 | H |
| 2-3-651 | O | N—Ph | H | H | a5 | H |
| 2-3-652 | O | N—Ph | t-bu | H | a5 | H |
| 2-3-653 | O | N—Ph | methyl | H | a5 | H |
| 2-3-654 | O | N—Ph | b1 | H | a5 | H |
| 2-3-655 | O | N—Ph | b2 | H | a5 | H |
| 2-3-656 | O | N—Ph | b3 | H | a5 | H |
| 2-3-657 | O | N—Ph | H | H | a6 | H |
| 2-3-658 | O | N—Ph | t-bu | H | a6 | H |
| 2-3-659 | O | N—Ph | methyl | H | a6 | H |
| 2-3-660 | O | N—Ph | b1 | H | a6 | H |
| 2-3-661 | O | N—Ph | b2 | H | a6 | H |
| 2-3-662 | O | N—Ph | b3 | H | a6 | H |
| 2-3-663 | O | N—Ph | H | H | b1 | H |
| 2-3-664 | O | N—Ph | t-bu | H | b1 | H |
| 2-3-665 | O | N—Ph | methyl | H | b1 | H |
| 2-3-666 | O | N—Ph | b1 | H | b1 | H |
| 2-3-667 | O | N—Ph | b2 | H | b1 | H |
| 2-3-668 | O | N—Ph | b3 | H | b1 | H |
| 2-3-669 | O | N—Ph | H | H | b2 | H |
| 2-3-670 | O | N—Ph | t-bu | H | b2 | H |
| 2-3-671 | O | N—Ph | methyl | H | b2 | H |
| 2-3-672 | O | N—Ph | b1 | H | b2 | H |
| 2-3-673 | O | N—Ph | b2 | H | b2 | H |
| 2-3-674 | O | N—Ph | b3 | H | b2 | H |
| 2-3-675 | O | N—Ph | H | H | b3 | H |
| 2-3-676 | O | N—Ph | t-bu | H | b3 | H |
| 2-3-677 | O | N—Ph | methyl | H | b3 | H |
| 2-3-678 | O | N—Ph | b1 | H | b3 | H |
| 2-3-679 | O | N—Ph | b2 | H | b3 | H |
| 2-3-680 | O | N—Ph | b3 | H | b3 | H |
| 2-3-681 | O | N—Ph | H | H | b4 | H |
| 2-3-682 | O | N—Ph | t-bu | H | b4 | H |
| 2-3-683 | O | N—Ph | methyl | H | b4 | H |
| 2-3-684 | O | N—Ph | b1 | H | b4 | H |
| 2-3-685 | O | N—Ph | b2 | H | b4 | H |
| 2-3-686 | O | N—Ph | b3 | H | b4 | H |
| 2-3-687 | O | N—Ph | H | H | b5 | H |
| 2-3-688 | O | N—Ph | t-bu | H | b5 | H |
| 2-3-689 | O | N—Ph | methyl | H | b5 | H |
| 2-3-690 | O | N—Ph | b1 | H | b5 | H |
| 2-3-691 | O | N—Ph | b2 | H | b5 | H |
| 2-3-692 | O | N—Ph | b3 | H | b5 | H |
| 2-3-693 | O | N—Ph | H | H | b6 | H |
| 2-3-694 | O | N—Ph | t-bu | H | b6 | H |
| 2-3-695 | O | N—Ph | methyl | H | b6 | H |
| 2-3-696 | O | N—Ph | b1 | H | b6 | H |
| 2-3-697 | O | N—Ph | b2 | H | b6 | H |
| 2-3-698 | O | N—Ph | b3 | H | b6 | H |
| 2-3-699 | O | N—Ph | H | H | b7 | H |
| 2-3-700 | O | N—Ph | t-bu | H | b7 | H |
| 2-3-701 | O | N—Ph | methyl | H | b7 | H |
| 2-3-702 | O | N—Ph | b1 | H | b7 | H |
| 2-3-703 | O | N—Ph | b2 | H | b7 | H |
| 2-3-704 | O | N—Ph | b3 | H | b7 | H |
| 2-3-705 | O | N—Ph | H | H | b8 | H |
| 2-3-706 | O | N—Ph | t-bu | H | b8 | H |
| 2-3-707 | O | N—Ph | methyl | H | b8 | H |
| 2-3-708 | O | N—Ph | b1 | H | b8 | H |
| 2-3-709 | O | N—Ph | b2 | H | b8 | H |
| 2-3-710 | O | N—Ph | b3 | H | b8 | H |
| 2-3-711 | O | N—Ph | H | H | b9 | H |
| 2-3-712 | O | N—Ph | t-bu | H | b9 | H |
| 2-3-713 | O | N—Ph | methyl | H | b9 | H |
| 2-3-714 | O | N—Ph | b1 | H | b9 | H |
| 2-3-715 | O | N—Ph | b2 | H | b9 | H |
| 2-3-716 | O | N—Ph | b3 | H | b9 | H |
| 2-3-717 | O | N—Ph | H | H | b10 | H |
| 2-3-718 | O | N—Ph | t-bu | H | b10 | H |
| 2-3-719 | O | N—Ph | methyl | H | b10 | H |
| 2-3-720 | O | N—Ph | b1 | H | b10 | H |
| 2-3-721 | O | N—Ph | b2 | H | b10 | H |
| 2-3-722 | O | N—Ph | b3 | H | b10 | H |
| 2-3-723 | O | N—Ph | H | H | a1 | a1 |
| 2-3-724 | O | N—Ph | t-bu | H | a1 | a1 |
| 2-3-725 | O | N—Ph | methyl | H | a1 | a1 |
| 2-3-726 | O | N—Ph | b1 | H | a1 | a1 |
| 2-3-727 | O | N—Ph | b2 | H | a1 | a1 |
| 2-3-728 | O | N—Ph | b3 | H | a1 | a1 |
| 2-3-729 | O | N—Ph | H | H | a2 | a2 |
| 2-3-730 | O | N—Ph | t-bu | H | a2 | a2 |
| 2-3-731 | O | N—Ph | methyl | H | a2 | a2 |
| 2-3-732 | O | N—Ph | b1 | H | a2 | a2 |
| 2-3-733 | O | N—Ph | b2 | H | a2 | a2 |
| 2-3-734 | O | N—Ph | b3 | H | a2 | a2 |
| 2-3-735 | O | N—Ph | H | H | a3 | a3 |
| 2-3-736 | O | N—Ph | t-bu | H | a3 | a3 |
| 2-3-737 | O | N—Ph | methyl | H | a3 | a3 |
| 2-3-738 | O | N—Ph | b1 | H | a3 | a3 |
| 2-3-739 | O | N—Ph | b2 | H | a3 | a3 |
| 2-3-740 | O | N—Ph | b3 | H | a3 | a3 |
| 2-3-741 | O | N—Ph | H | H | b1 | b1 |
| 2-3-742 | O | N—Ph | t-bu | H | b1 | b1 |
| 2-3-743 | O | N—Ph | methyl | H | b1 | b1 |
| 2-3-744 | O | N—Ph | b1 | H | b1 | b1 |
| 2-3-745 | O | N—Ph | b2 | H | b1 | b1 |
| 2-3-746 | O | N—Ph | b3 | H | b1 | b1 |
| 2-3-747 | O | N—Ph | H | H | b2 | b2 |
| 2-3-748 | O | N—Ph | t-bu | H | b2 | b2 |
| 2-3-749 | O | N—Ph | methyl | H | b2 | b2 |
| 2-3-750 | O | N—Ph | b1 | H | b2 | b2 |
| 2-3-751 | O | N—Ph | b2 | H | b2 | b2 |
| 2-3-752 | O | N—Ph | b3 | H | b2 | b2 |
| 2-3-753 | O | N—Ph | H | H | b3 | b3 |
| 2-3-754 | O | N—Ph | t-bu | H | b3 | b3 |
| 2-3-755 | O | N—Ph | methyl | H | b3 | b3 |
| 2-3-756 | O | N—Ph | b1 | H | b3 | b3 |
| 2-3-757 | O | N—Ph | b2 | H | b3 | b3 |
| 2-3-758 | O | N—Ph | b3 | H | b3 | b3 |
| 2-3-759 | O | N—Ph | H | H | b5 | b5 |
| 2-3-760 | O | N—Ph | t-bu | H | b5 | b5 |
| 2-3-761 | O | N—Ph | methyl | H | b5 | b5 |
| 2-3-762 | O | N—Ph | b1 | H | b5 | b5 |
| 2-3-763 | O | N—Ph | b2 | H | b5 | b5 |
| 2-3-764 | O | N—Ph | b3 | H | b5 | b5 |
| 2-3-765 | O | N—Ph | H | H | b6 | b6 |
| 2-3-766 | O | N—Ph | t-bu | H | b6 | b6 |
| 2-3-767 | O | N—Ph | methyl | H | b6 | b6 |
| 2-3-768 | O | N—Ph | b1 | H | b6 | b6 |
| 2-3-769 | O | N—Ph | b2 | H | b6 | b6 |
| 2-3-770 | O | N—Ph | b3 | H | b6 | b6 |
| 2-3-771 | O | N—Ph | H | H | b7 | b7 |
| 2-3-772 | O | N—Ph | t-bu | H | b7 | b7 |
| 2-3-773 | O | N—Ph | methyl | H | b7 | b7 |
| 2-3-774 | O | N—Ph | b1 | H | b7 | b7 |
| 2-3-775 | O | N—Ph | b2 | H | b7 | b7 |
| 2-3-776 | O | N—Ph | b3 | H | b7 | b7 |
| 2-3-777 | S | N—Ph | H | H | H | H |
| 2-3-778 | S | N—Ph | t-bu | H | H | H |
| 2-3-779 | S | N—Ph | methyl | H | H | H |
| 2-3-780 | S | N—Ph | t-bu | t-bu | t-bu | t-bu |
| 2-3-781 | S | N—Ph | methyl | methyl | methyl | methyl |
| 2-3-782 | S | N—Ph | b1 | H | H | H |
| 2-3-783 | S | N—Ph | b2 | H | H | H |
| 2-3-784 | S | N—Ph | b3 | H | H | H |
| 2-3-785 | S | N—Ph | H | H | H | a1 |
| 2-3-786 | S | N—Ph | t-bu | H | H | a1 |
| 2-3-787 | S | N—Ph | methyl | H | H | a1 |
| 2-3-788 | S | N—Ph | b1 | H | H | a1 |
| 2-3-789 | S | N—Ph | b2 | H | H | a1 |
| 2-3-790 | S | N—Ph | b3 | H | H | a1 |
| 2-3-791 | S | N—Ph | H | H | H | a2 |
| 2-3-792 | S | N—Ph | t-bu | H | H | a2 |
| 2-3-793 | S | N—Ph | methyl | H | H | a2 |
| 2-3-794 | S | N—Ph | b1 | H | H | a2 |
| 2-3-795 | S | N—Ph | b2 | H | H | a2 |
| 2-3-796 | S | N—Ph | b3 | H | H | a2 |
| 2-3-797 | S | N—Ph | H | H | H | a3 |
| 2-3-798 | S | N—Ph | t-bu | H | H | a3 |
| 2-3-799 | S | N—Ph | methyl | H | H | a3 |
| 2-3-800 | S | N—Ph | b1 | H | H | a3 |
| 2-3-801 | S | N—Ph | b2 | H | H | a3 |
| 2-3-802 | S | N—Ph | b3 | H | H | a3 |
| 2-3-803 | S | N—Ph | H | H | H | a4 |
| 2-3-804 | S | N—Ph | t-bu | H | H | a4 |
| 2-3-805 | S | N—Ph | methyl | H | H | a4 |
| 2-3-806 | S | N—Ph | b1 | H | H | a4 |
| 2-3-807 | S | N—Ph | b2 | H | H | a4 |
| 2-3-808 | S | N—Ph | b3 | H | H | a4 |
| 2-3-809 | S | N—Ph | H | H | H | a5 |
| 2-3-810 | S | N—Ph | t-bu | H | H | a5 |
| 2-3-811 | S | N—Ph | methyl | H | H | a5 |
| 2-3-812 | S | N—Ph | b1 | H | H | a5 |
| 2-3-813 | S | N—Ph | b2 | H | H | a5 |
| 2-3-814 | S | N—Ph | b3 | H | H | a5 |
| 2-3-815 | S | N—Ph | H | H | H | a6 |
| 2-3-816 | S | N—Ph | t-bu | H | H | a6 |
| 2-3-817 | S | N—Ph | methyl | H | H | a6 |
| 2-3-818 | S | N—Ph | b1 | H | H | a6 |
| 2-3-819 | S | N—Ph | b2 | H | H | a6 |
| 2-3-820 | S | N—Ph | b3 | H | H | a6 |
| 2-3-821 | S | N—Ph | H | H | a1 | H |
| 2-3-822 | S | N—Ph | t-bu | H | a1 | H |
| 2-3-823 | S | N—Ph | methyl | H | a1 | H |
| 2-3-824 | S | N—Ph | b1 | H | a1 | H |
| 2-3-825 | S | N—Ph | b2 | H | a1 | H |
| 2-3-826 | S | N—Ph | b3 | H | a1 | H |
| 2-3-827 | S | N—Ph | H | H | a2 | H |
| 2-3-828 | S | N—Ph | t-bu | H | a2 | H |
| 2-3-829 | S | N—Ph | methyl | H | a2 | H |
| 2-3-830 | S | N—Ph | b1 | H | a2 | H |
| 2-3-831 | S | N—Ph | b2 | H | a2 | H |
| 2-3-832 | S | N—Ph | b3 | H | a2 | H |
| 2-3-833 | S | N—Ph | H | H | a3 | H |
| 2-3-834 | S | N—Ph | t-bu | H | a3 | H |
| 2-3-835 | S | N—Ph | methyl | H | a3 | H |
| 2-3-836 | S | N—Ph | b1 | H | a3 | H |
| 2-3-837 | S | N—Ph | b2 | H | a3 | H |
| 2-3-838 | S | N—Ph | b3 | H | a3 | H |
| 2-3-839 | S | N—Ph | H | H | a4 | H |
| 2-3-840 | S | N—Ph | t-bu | H | a4 | H |
| 2-3-841 | S | N—Ph | methyl | H | a4 | H |
| 2-3-842 | S | N—Ph | b1 | H | a4 | H |
| 2-3-843 | S | N—Ph | b2 | H | a4 | H |
| 2-3-844 | S | N—Ph | b3 | H | a4 | H |
| 2-3-845 | S | N—Ph | H | H | a5 | H |
| 2-3-846 | S | N—Ph | t-bu | H | a5 | H |
| 2-3-847 | S | N—Ph | methyl | H | a5 | H |
| 2-3-848 | S | N—Ph | b1 | H | a5 | H |
| 2-3-849 | S | N—Ph | b2 | H | a5 | H |
| 2-3-850 | S | N—Ph | b3 | H | a5 | H |
| 2-3-851 | S | N—Ph | H | H | a6 | H |
| 2-3-852 | S | N—Ph | t-bu | H | a6 | H |
| 2-3-853 | S | N—Ph | methyl | H | a6 | H |
| 2-3-854 | S | N—Ph | b1 | H | a6 | H |
| 2-3-855 | S | N—Ph | b2 | H | a6 | H |
| 2-3-856 | S | N—Ph | b3 | H | a6 | H |
| 2-3-857 | S | N—Ph | H | H | b1 | H |
| 2-3-858 | S | N—Ph | t-bu | H | b1 | H |
| 2-3-859 | S | N—Ph | methyl | H | b1 | H |
| 2-3-860 | S | N—Ph | b1 | H | b1 | H |
| 2-3-861 | S | N—Ph | b2 | H | b1 | H |
| 2-3-862 | S | N—Ph | b3 | H | b1 | H |
| 2-3-863 | S | N—Ph | H | H | b2 | H |
| 2-3-864 | S | N—Ph | t-bu | H | b2 | H |
| 2-3-865 | S | N—Ph | methyl | H | b2 | H |
| 2-3-866 | S | N—Ph | b1 | H | b2 | H |
| 2-3-867 | S | N—Ph | b2 | H | b2 | H |
| 2-3-868 | S | N—Ph | b3 | H | b2 | H |
| 2-3-869 | S | N—Ph | H | H | b3 | H |
| 2-3-870 | S | N—Ph | t-bu | H | b3 | H |
| 2-3-871 | S | N—Ph | methyl | H | b3 | H |
| 2-3-872 | S | N—Ph | b1 | H | b3 | H |
| 2-3-873 | S | N—Ph | b2 | H | b3 | H |
| 2-3-874 | S | N—Ph | b3 | H | b3 | H |
| 2-3-875 | S | N—Ph | H | H | b4 | H |
| 2-3-876 | S | N—Ph | t-bu | H | b4 | H |
| 2-3-877 | S | N—Ph | methyl | H | b4 | H |
| 2-3-878 | S | N—Ph | b1 | H | b4 | H |
| 2-3-879 | S | N—Ph | b2 | H | b4 | H |
| 2-3-880 | S | N—Ph | b3 | H | b4 | H |
| 2-3-881 | S | N—Ph | H | H | b5 | H |
| 2-3-882 | S | N—Ph | t-bu | H | b5 | H |
| 2-3-883 | S | N—Ph | methyl | H | b5 | H |
| 2-3-884 | S | N—Ph | b1 | H | b5 | H |
| 2-3-885 | S | N—Ph | b2 | H | b5 | H |
| 2-3-886 | S | N—Ph | b3 | H | b5 | H |
| 2-3-887 | S | N—Ph | H | H | b6 | H |
| 2-3-888 | S | N—Ph | t-bu | H | b6 | H |
| 2-3-889 | S | N—Ph | methyl | H | b6 | H |
| 2-3-890 | S | N—Ph | b1 | H | b6 | H |
| 2-3-891 | S | N—Ph | b2 | H | b6 | H |
| 2-3-892 | S | N—Ph | b3 | H | b6 | H |
| 2-3-893 | S | N—Ph | H | H | b7 | H |
| 2-3-894 | S | N—Ph | t-bu | H | b7 | H |
| 2-3-895 | S | N—Ph | methyl | H | b7 | H |
| 2-3-896 | S | N—Ph | b1 | H | b7 | H |
| 2-3-897 | S | N—Ph | b2 | H | b7 | H |
| 2-3-898 | S | N—Ph | b3 | H | b7 | H |
| 2-3-899 | S | N—Ph | H | H | b8 | H |
| 2-3-900 | S | N—Ph | t-bu | H | b8 | H |
| 2-3-901 | S | N—Ph | methyl | H | b8 | H |
| 2-3-902 | S | N—Ph | b1 | H | b8 | H |
| 2-3-903 | S | N—Ph | b2 | H | b8 | H |
| 2-3-904 | S | N—Ph | b3 | H | b8 | H |
| 2-3-905 | S | N—Ph | H | H | b9 | H |
| 2-3-906 | S | N—Ph | t-bu | H | b9 | H |
| 2-3-907 | S | N—Ph | methyl | H | b9 | H |
| 2-3-908 | S | N—Ph | b1 | H | b9 | H |
| 2-3-909 | S | N—Ph | b2 | H | b9 | H |
| 2-3-910 | S | N—Ph | b3 | H | b9 | H |
| 2-3-911 | S | N—Ph | H | H | b10 | H |
| 2-3-912 | S | N—Ph | t-bu | H | b10 | H |
| 2-3-913 | S | N—Ph | methyl | H | b10 | H |
| 2-3-914 | S | N—Ph | b1 | H | b10 | H |
| 2-3-915 | S | N—Ph | b2 | H | b10 | H |
| 2-3-916 | S | N—Ph | b3 | H | b10 | H |
| 2-3-917 | S | N—Ph | H | H | a1 | a1 |
| 2-3-918 | S | N—Ph | t-bu | H | a1 | a1 |
| 2-3-919 | S | N—Ph | methyl | H | a1 | a1 |
| 2-3-920 | S | N—Ph | b1 | H | a1 | a1 |
| 2-3-921 | S | N—Ph | b2 | H | a1 | a1 |
| 2-3-922 | S | N—Ph | b3 | H | a1 | a1 |
| 2-3-923 | S | N—Ph | H | H | a2 | a2 |
| 2-3-924 | S | N—Ph | t-bu | H | a2 | a2 |
| 2-3-925 | S | N—Ph | methyl | H | a2 | a2 |
| 2-3-926 | S | N—Ph | b1 | H | a2 | a2 |
| 2-3-927 | S | N—Ph | b2 | H | a2 | a2 |
| 2-3-928 | S | N—Ph | b3 | H | a2 | a2 |
| 2-3-929 | S | N—Ph | H | H | a3 | a3 |
| 2-3-930 | S | N—Ph | t-bu | H | a3 | a3 |
| 2-3-931 | S | N—Ph | methyl | H | a3 | a3 |
| 2-3-932 | S | N—Ph | b1 | H | a3 | a3 |
| 2-3-933 | S | N—Ph | b2 | H | a3 | a3 |
| 2-3-934 | S | N—Ph | b3 | H | a3 | a3 |
| 2-3-935 | S | N—Ph | H | H | b1 | b1 |
| 2-3-936 | S | N—Ph | t-bu | H | b1 | b1 |
| 2-3-937 | S | N—Ph | methyl | H | b1 | b1 |
| 2-3-938 | S | N—Ph | b1 | H | b1 | b1 |
| 2-3-939 | S | N—Ph | b2 | H | b1 | b1 |
| 2-3-940 | S | N—Ph | b3 | H | b1 | b1 |
| 2-3-941 | S | N—Ph | H | H | b2 | b2 |
| 2-3-942 | S | N—Ph | t-bu | H | b2 | b2 |
| 2-3-943 | S | N—Ph | methyl | H | b2 | b2 |
| 2-3-944 | S | N—Ph | b1 | H | b2 | b2 |
| 2-3-945 | S | N—Ph | b2 | H | b2 | b2 |
| 2-3-946 | S | N—Ph | b3 | H | b2 | b2 |
| 2-3-947 | S | N—Ph | H | H | b3 | b3 |
| 2-3-948 | S | N—Ph | t-bu | H | b3 | b3 |
| 2-3-949 | S | N—Ph | methyl | H | b3 | b3 |
| 2-3-950 | S | N—Ph | b1 | H | b3 | b3 |
| 2-3-951 | S | N—Ph | b2 | H | b3 | b3 |
| 2-3-952 | S | N—Ph | b3 | H | b3 | b3 |
| 2-3-953 | S | N—Ph | H | H | b5 | b5 |
| 2-3-954 | S | N—Ph | t-bu | H | b5 | b5 |
| 2-3-955 | S | N—Ph | methyl | H | b5 | b5 |
| 2-3-956 | S | N—Ph | b1 | H | b5 | b5 |
| 2-3-957 | S | N—Ph | b2 | H | b5 | b5 |
| 2-3-958 | S | N—Ph | b3 | H | b5 | b5 |
| 2-3-959 | S | N—Ph | H | H | b6 | b6 |
| 2-3-960 | S | N—Ph | t-bu | H | b6 | b6 |
| 2-3-961 | S | N—Ph | methyl | H | b6 | b6 |
| 2-3-962 | S | N—Ph | b1 | H | b6 | b6 |
| 2-3-963 | S | N—Ph | b2 | H | b6 | b6 |
| 2-3-964 | S | N—Ph | b3 | H | b6 | b6 |
| 2-3-965 | S | N—Ph | H | H | b7 | b7 |
| 2-3-966 | S | N—Ph | t-bu | H | b7 | b7 |
| 2-3-967 | S | N—Ph | methyl | H | b7 | b7 |
| 2-3-968 | S | N—Ph | b1 | H | b7 | b7 |
| 2-3-969 | S | N—Ph | b2 | H | b7 | b7 |
| 2-3-970 | S | N—Ph | b3 | H | b7 | b7 |

Group 2-4
The compounds are represented by the formula 2-4 below, wherein CY₁ and CY₃ are each a benzene group, X₁ is B, and Y1, Y2, R2a, R2c, (R1)a1, (R3)a3 are each shown in the table 4 below, 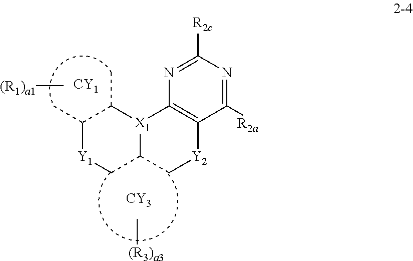
TABLE 4

| Compound | | | | | | |
|---|---|---|---|---|---|---|
| No. | Y1 | Y2 | R2a | R2c | (R1)a1 | (R3)a3 |
| 2-4-001 | N—Ph | N—Ph | H | H | H | H |
| 2-4-002 | N—Ph | N—Ph | t-bu | t-bu | H | H |
| 2-4-003 | N—Ph | N—Ph | methyl | methyl | H | H |
| 2-4-004 | N—Ph | N—Ph | t-bu | t-bu | t-bu | t-bu |
| 2-4-005 | N—Ph | N—Ph | methyl | methyl | methyl | methyl |
| 2-4-006 | N—Ph | N—Ph | b1 | b1 | H | H |
| 2-4-007 | N—Ph | N—Ph | b2 | b2 | H | H |
| 2-4-008 | N—Ph | N—Ph | b3 | b3 | H | H |
| 2-4-009 | N—Ph | N—Ph | H | H | H | a1 |
| 2-4-010 | N—Ph | N—Ph | t-bu | t-bu | H | a1 |
| 2-4-011 | N—Ph | N—Ph | methyl | methyl | H | a1 |
| 2-4-012 | N—Ph | N—Ph | b1 | b1 | H | a1 |
| 2-4-013 | N—Ph | N—Ph | b2 | b2 | H | a1 |
| 2-4-014 | N—Ph | N—Ph | b3 | b3 | H | a1 |
| 2-4-015 | N—Ph | N—Ph | H | H | H | a2 |
| 2-4-016 | N—Ph | N—Ph | t-bu | t-bu | H | a2 |
| 2-4-017 | N—Ph | N—Ph | methyl | methyl | H | a2 |
| 2-4-018 | N—Ph | N—Ph | b1 | b1 | H | a2 |
| 2-4-019 | N—Ph | N—Ph | b2 | b2 | H | a2 |
| 2-4-020 | N—Ph | N—Ph | b3 | b3 | H | a2 |
| 2-4-021 | N—Ph | N—Ph | H | H | H | a3 |
| 2-4-022 | N—Ph | N—Ph | t-bu | t-bu | H | a3 |
| 2-4-023 | N—Ph | N—Ph | methyl | methyl | H | a3 |
| 2-4-024 | N—Ph | N—Ph | b1 | b1 | H | a3 |
| 2-4-025 | N—Ph | N—Ph | b2 | b2 | H | a3 |
| 2-4-026 | N—Ph | N—Ph | b3 | b3 | H | a3 |
| 2-4-027 | N—Ph | N—Ph | H | H | H | a4 |
| 2-4-028 | N—Ph | N—Ph | t-bu | t-bu | H | a4 |
| 2-4-029 | N—Ph | N—Ph | methyl | methyl | H | a4 |
| 2-4-030 | N—Ph | N—Ph | b1 | b1 | H | a4 |
| 2-4-031 | N—Ph | N—Ph | b2 | b2 | H | a4 |
| 2-4-032 | N—Ph | N—Ph | b3 | b3 | H | a4 |
| 2-4-033 | N—Ph | N—Ph | H | H | H | a5 |
| 2-4-034 | N—Ph | N—Ph | t-bu | t-bu | H | a5 |
| 2-4-035 | N—Ph | N—Ph | methyl | methyl | H | a5 |
| 2-4-036 | N—Ph | N—Ph | b1 | b1 | H | a5 |
| 2-4-037 | N—Ph | N—Ph | b2 | b2 | H | a5 |
| 2-4-038 | N—Ph | N—Ph | b3 | b3 | H | a5 |
| 2-4-039 | N—Ph | N—Ph | H | H | H | a6 |
| 2-4-040 | N—Ph | N—Ph | t-bu | t-bu | H | a6 |
| 2-4-041 | N—Ph | N—Ph | methyl | methyl | H | a6 |
| 2-4-042 | N—Ph | N—Ph | b1 | b1 | H | a6 |
| 2-4-043 | N—Ph | N—Ph | b2 | b2 | H | a6 |
| 2-4-044 | N—Ph | N—Ph | b3 | b3 | H | a6 |
| 2-4-045 | N—Ph | N—Ph | H | H | a1 | H |
| 2-4-046 | N—Ph | N—Ph | t-bu | t-bu | a1 | H |
| 2-4-047 | N—Ph | N—Ph | methyl | methyl | a1 | H |
| 2-4-048 | N—Ph | N—Ph | b1 | b1 | a1 | H |
| 2-4-049 | N—Ph | N—Ph | b2 | b2 | a1 | H |
| 2-4-050 | N—Ph | N—Ph | b3 | b3 | a1 | H |
| 2-4-051 | N—Ph | N—Ph | H | H | a2 | H |
| 2-4-052 | N—Ph | N—Ph | t-bu | t-bu | a2 | H |
| 2-4-053 | N—Ph | N—Ph | methyl | methyl | a2 | H |
| 2-4-054 | N—Ph | N—Ph | b1 | b1 | a2 | H |
| 2-4-055 | N—Ph | N—Ph | b2 | b2 | a2 | H |
| 2-4-056 | N—Ph | N—Ph | b3 | b3 | a2 | H |
| 2-4-057 | N—Ph | N—Ph | H | H | a3 | H |
| 2-4-058 | N—Ph | N—Ph | t-bu | t-bu | a3 | H |
| 2-4-059 | N—Ph | N—Ph | methyl | methyl | a3 | H |
| 2-4-060 | N—Ph | N—Ph | b1 | b1 | a3 | H |
| 2-4-061 | N—Ph | N—Ph | b2 | b2 | a3 | H |
| 2-4-062 | N—Ph | N—Ph | b3 | b3 | a3 | H |
| 2-4-063 | N—Ph | N—Ph | H | H | a4 | H |
| 2-4-064 | N—Ph | N—Ph | t-bu | t-bu | a4 | H |
| 2-4-065 | N—Ph | N—Ph | methyl | methyl | a4 | H |
| 2-4-066 | N—Ph | N—Ph | b1 | b1 | a4 | H |
| 2-4-067 | N—Ph | N—Ph | b2 | b2 | a4 | H |
| 2-4-068 | N—Ph | N—Ph | b3 | b3 | a4 | H |
| 2-4-069 | N—Ph | N—Ph | H | H | a5 | H |
| 2-4-070 | N—Ph | N—Ph | t-bu | t-bu | a5 | H |
| 2-4-071 | N—Ph | N—Ph | methyl | methyl | a5 | H |
| 2-4-072 | N—Ph | N—Ph | b1 | b1 | a5 | H |
| 2-4-073 | N—Ph | N—Ph | b2 | b2 | a5 | H |
| 2-4-074 | N—Ph | N—Ph | b3 | b3 | a5 | H |
| 2-4-075 | N—Ph | N—Ph | H | H | a6 | H |
| 2-4-076 | N—Ph | N—Ph | t-bu | t-bu | a6 | H |
| 2-4-077 | N—Ph | N—Ph | methyl | methyl | a6 | H |
| 2-4-078 | N—Ph | N—Ph | b1 | b1 | a6 | H |
| 2-4-079 | N—Ph | N—Ph | b2 | b2 | a6 | H |
| 2-4-080 | N—Ph | N—Ph | b3 | b3 | a6 | H |
| 2-4-081 | N—Ph | N—Ph | H | H | b1 | H |
| 2-4-082 | N—Ph | N—Ph | t-bu | t-bu | b1 | H |
| 2-4-083 | N—Ph | N—Ph | methyl | methyl | b1 | H |
| 2-4-084 | N—Ph | N—Ph | b1 | b1 | b1 | H |
| 2-4-085 | N—Ph | N—Ph | b2 | b2 | b1 | H |
| 2-4-086 | N—Ph | N—Ph | b3 | b3 | b1 | H |
| 2-4-087 | N—Ph | N—Ph | H | H | b2 | H |
| 2-4-088 | N—Ph | N—Ph | t-bu | t-bu | b2 | H |
| 2-4-089 | N—Ph | N—Ph | methyl | methyl | b2 | H |
| 2-4-090 | N—Ph | N—Ph | b1 | b1 | b2 | H |
| 2-4-091 | N—Ph | N—Ph | b2 | b2 | b2 | H |
| 2-4-092 | N—Ph | N—Ph | b3 | b3 | b2 | H |
| 2-4-093 | N—Ph | N—Ph | H | H | b3 | H |
| 2-4-094 | N—Ph | N—Ph | t-bu | t-bu | b3 | H |
| 2-4-095 | N—Ph | N—Ph | methyl | methyl | b3 | H |
| 2-4-096 | N—Ph | N—Ph | b1 | b1 | b3 | H |
| 2-4-097 | N—Ph | N—Ph | b2 | b2 | b3 | H |
| 2-4-098 | N—Ph | N—Ph | b3 | b3 | b3 | H |
| 2-4-099 | N—Ph | N—Ph | H | H | b4 | H |
| 2-4-100 | N—Ph | N—Ph | t-bu | t-bu | b4 | H |
| 2-4-101 | N—Ph | N—Ph | methyl | methyl | b4 | H |
| 2-4-102 | N—Ph | N—Ph | b1 | b1 | b4 | H |
| 2-4-103 | N—Ph | N—Ph | b2 | b2 | b4 | H |
| 2-4-104 | N—Ph | N—Ph | b3 | b3 | b4 | H |
| 2-4-105 | N—Ph | N—Ph | H | H | b5 | H |
| 2-4-106 | N—Ph | N—Ph | t-bu | t-bu | b5 | H |
| 2-4-107 | N—Ph | N—Ph | methyl | methyl | b5 | H |
| 2-4-108 | N—Ph | N—Ph | b1 | b1 | b5 | H |
| 2-4-109 | N—Ph | N—Ph | b2 | b2 | b5 | H |
| 2-4-110 | N—Ph | N—Ph | b3 | b3 | b5 | H |
| 2-4-111 | N—Ph | N—Ph | H | H | b6 | H |
| 2-4-112 | N—Ph | N—Ph | t-bu | t-bu | b6 | H |
| 2-4-113 | N—Ph | N—Ph | methyl | methyl | b6 | H |
| 2-4-114 | N—Ph | N—Ph | b1 | b1 | b6 | H |
| 2-4-115 | N—Ph | N—Ph | b2 | b2 | b6 | H |
| 2-4-116 | N—Ph | N—Ph | b3 | b3 | b6 | H |
| 2-4-117 | N—Ph | N—Ph | H | H | b7 | H |
| 2-4-118 | N—Ph | N—Ph | t-bu | t-bu | b7 | H |
| 2-4-119 | N—Ph | N—Ph | methyl | methyl | b7 | H |
| 2-4-120 | N—Ph | N—Ph | b1 | b1 | b7 | H |
| 2-4-121 | N—Ph | N—Ph | b2 | b2 | b7 | H |
| 2-4-122 | N—Ph | N—Ph | b3 | b3 | b7 | H |
| 2-4-123 | N—Ph | N—Ph | H | H | b8 | H |
| 2-4-124 | N—Ph | N—Ph | t-bu | t-bu | b8 | H |
| 2-4-125 | N—Ph | N—Ph | methyl | methyl | b8 | H |
| 2-4-126 | N—Ph | N—Ph | b1 | b1 | b8 | H |
| 2-4-127 | N—Ph | N—Ph | b2 | b2 | b8 | H |
| 2-4-128 | N—Ph | N—Ph | b3 | b3 | b8 | H |
| 2-4-129 | N—Ph | N—Ph | H | H | b9 | H |
| 2-4-130 | N—Ph | N—Ph | t-bu | t-bu | b9 | H |
| 2-4-131 | N—Ph | N—Ph | methyl | methyl | b9 | H |
| 2-4-132 | N—Ph | N—Ph | b1 | b1 | b9 | H |
| 2-4-133 | N—Ph | N—Ph | b2 | b2 | b9 | H |
| 2-4-134 | N—Ph | N—Ph | b3 | b3 | b9 | H |
| 2-4-135 | N—Ph | N—Ph | H | H | b10 | H |
| 2-4-136 | N—Ph | N—Ph | t-bu | t-bu | b10 | H |
| 2-4-137 | N—Ph | N—Ph | methyl | methyl | b10 | H |
| 2-4-138 | N—Ph | N—Ph | b1 | b1 | b10 | H |
| 2-4-139 | N—Ph | N—Ph | b2 | b2 | b10 | H |
| 2-4-140 | N—Ph | N—Ph | b3 | b3 | b10 | H |
| 2-4-141 | N—Ph | N—Ph | H | H | a1 | a1 |
| 2-4-142 | N—Ph | N—Ph | t-bu | t-bu | a1 | a1 |
| 2-4-143 | N—Ph | N—Ph | methyl | methyl | a1 | a1 |
| 2-4-144 | N—Ph | N—Ph | b1 | b1 | a1 | a1 |
| 2-4-145 | N—Ph | N—Ph | b2 | b2 | a1 | a1 |
| 2-4-146 | N—Ph | N—Ph | b3 | b3 | a1 | a1 |
| 2-4-147 | N—Ph | N—Ph | H | H | a2 | a2 |
| 2-4-148 | N—Ph | N—Ph | t-bu | t-bu | a2 | a2 |
| 2-4-149 | N—Ph | N—Ph | methyl | methyl | a2 | a2 |
| 2-4-150 | N—Ph | N—Ph | b1 | b1 | a2 | a2 |
| 2-4-151 | N—Ph | N—Ph | b2 | b2 | a2 | a2 |
| 2-4-152 | N—Ph | N—Ph | b3 | b3 | a2 | a2 |
| 2-4-153 | N—Ph | N—Ph | H | H | a3 | a3 |
| 2-4-154 | N—Ph | N—Ph | t-bu | t-bu | a3 | a3 |
| 2-4-155 | N—Ph | N—Ph | methyl | methyl | a3 | a3 |
| 2-4-156 | N—Ph | N—Ph | b1 | b1 | a3 | a3 |
| 2-4-157 | N—Ph | N—Ph | b2 | b2 | a3 | a3 |
| 2-4-158 | N—Ph | N—Ph | b3 | b3 | a3 | a3 |
| 2-4-159 | N—Ph | N—Ph | H | H | b1 | b1 |
| 2-4-160 | N—Ph | N—Ph | t-bu | t-bu | b1 | b1 |
| 2-4-161 | N—Ph | N—Ph | methyl | methyl | b1 | b1 |
| 2-4-162 | N—Ph | N—Ph | b1 | b1 | b1 | b1 |
| 2-4-163 | N—Ph | N—Ph | b2 | b2 | b1 | b1 |
| 2-4-164 | N—Ph | N—Ph | b3 | b3 | b1 | b1 |
| 2-4-165 | N—Ph | N—Ph | H | H | b2 | b2 |
| 2-4-166 | N—Ph | N—Ph | t-bu | t-bu | b2 | b2 |
| 2-4-167 | N—Ph | N—Ph | methyl | methyl | b2 | b2 |
| 2-4-168 | N—Ph | N—Ph | b1 | b1 | b2 | b2 |
| 2-4-169 | N—Ph | N—Ph | b2 | b2 | b2 | b2 |
| 2-4-170 | N—Ph | N—Ph | b3 | b3 | b2 | b2 |
| 2-4-171 | N—Ph | N—Ph | H | H | b3 | b3 |
| 2-4-172 | N—Ph | N—Ph | t-bu | t-bu | b3 | b3 |
| 2-4-173 | N—Ph | N—Ph | methyl | methyl | b3 | b3 |
| 2-4-174 | N—Ph | N—Ph | b1 | b1 | b3 | b3 |
| 2-4-175 | N—Ph | N—Ph | b2 | b2 | b3 | b3 |
| 2-4-176 | N—Ph | N—Ph | b3 | b3 | b3 | b3 |
| 2-4-177 | N—Ph | N—Ph | H | H | b5 | b5 |
| 2-4-178 | N—Ph | N—Ph | t-bu | t-bu | b5 | b5 |
| 2-4-179 | N—Ph | N—Ph | methyl | methyl | b5 | b5 |
| 2-4-180 | N—Ph | N—Ph | b1 | b1 | b5 | b5 |
| 2-4-181 | N—Ph | N—Ph | b2 | b2 | b5 | b5 |
| 2-4-182 | N—Ph | N—Ph | b3 | b3 | b5 | b5 |
| 2-4-183 | N—Ph | N—Ph | H | H | b6 | b6 |
| 2-4-184 | N—Ph | N—Ph | t-bu | t-bu | b6 | b6 |
| 2-4-185 | N—Ph | N—Ph | methyl | methyl | b6 | b6 |
| 2-4-186 | N—Ph | N—Ph | b1 | b1 | b6 | b6 |
| 2-4-187 | N—Ph | N—Ph | b2 | b2 | b6 | b6 |
| 2-4-188 | N—Ph | N—Ph | b3 | b3 | b6 | b6 |
| 2-4-189 | N—Ph | N—Ph | H | H | b7 | b7 |
| 2-4-190 | N—Ph | N—Ph | t-bu | t-bu | b7 | b7 |
| 2-4-191 | N—Ph | N—Ph | methyl | methyl | b7 | b7 |
| 2-4-192 | N—Ph | N—Ph | b1 | b1 | b7 | b7 |
| 2-4-193 | N—Ph | N—Ph | b2 | b2 | b7 | b7 |
| 2-4-194 | N—Ph | N—Ph | b3 | b3 | b7 | b7 |
| 2-4-195 | N-tolyl | N-tolyl | H | H | H | H |
| 2-4-196 | N-tolyl | N-tolyl | t-bu | t-bu | H | H |
| 2-4-197 | N-tolyl | N-tolyl | methyl | methyl | H | H |
| 2-4-198 | N-tolyl | N-tolyl | t-bu | t-bu | t-bu | t-bu |
| 2-4-199 | N-tolyl | N-tolyl | methyl | methyl | methyl | methyl |
| 2-4-200 | N-tolyl | N-tolyl | b1 | b1 | H | H |
| 2-4-201 | N-tolyl | N-tolyl | b2 | b2 | H | H |
| 2-4-202 | N-tolyl | N-tolyl | b3 | b3 | H | H |
| 2-4-203 | N-tolyl | N-tolyl | H | H | H | a1 |
| 2-4-204 | N-tolyl | N-tolyl | t-bu | t-bu | H | a1 |
| 2-4-205 | N-tolyl | N-tolyl | methyl | methyl | H | a1 |
| 2-4-206 | N-tolyl | N-tolyl | b1 | b1 | H | a1 |
| 2-4-207 | N-tolyl | N-tolyl | b2 | b2 | H | a1 |
| 2-4-208 | N-tolyl | N-tolyl | b3 | b3 | H | a1 |
| 2-4-209 | N-tolyl | N-tolyl | H | H | H | a2 |
| 2-4-210 | N-tolyl | N-tolyl | t-bu | t-bu | H | a2 |
| 2-4-211 | N-tolyl | N-tolyl | methyl | methyl | H | a2 |
| 2-4-212 | N-tolyl | N-tolyl | b1 | b1 | H | a2 |
| 2-4-213 | N-tolyl | N-tolyl | b2 | b2 | H | a2 |
| 2-4-214 | N-tolyl | N-tolyl | b3 | b3 | H | a2 |
| 2-4-215 | N-tolyl | N-tolyl | H | H | H | a3 |
| 2-4-216 | N-tolyl | N-tolyl | t-bu | t-bu | H | a3 |
| 2-4-217 | N-tolyl | N-tolyl | methyl | methyl | H | a3 |
| 2-4-218 | N-tolyl | N-tolyl | b1 | b1 | H | a3 |
| 2-4-219 | N-tolyl | N-tolyl | b2 | b2 | H | a3 |
| 2-4-220 | N-tolyl | N-tolyl | b3 | b3 | H | a3 |
| 2-4-221 | N-tolyl | N-tolyl | H | H | H | a4 |
| 2-4-222 | N-tolyl | N-tolyl | t-bu | t-bu | H | a4 |
| 2-4-223 | N-tolyl | N-tolyl | methyl | methyl | H | a4 |
| 2-4-224 | N-tolyl | N-tolyl | b1 | b1 | H | a4 |
| 2-4-225 | N-tolyl | N-tolyl | b2 | b2 | H | a4 |
| 2-4-226 | N-tolyl | N-tolyl | b3 | b3 | H | a4 |
| 2-4-227 | N-tolyl | N-tolyl | H | H | H | a5 |
| 2-4-228 | N-tolyl | N-tolyl | t-bu | t-bu | H | a5 |
| 2-4-229 | N-tolyl | N-tolyl | methyl | methyl | H | a5 |
| 2-4-230 | N-tolyl | N-tolyl | b1 | b1 | H | a5 |
| 2-4-231 | N-tolyl | N-tolyl | b2 | b2 | H | a5 |
| 2-4-232 | N-tolyl | N-tolyl | b3 | b3 | H | a5 |
| 2-4-233 | N-tolyl | N-tolyl | H | H | H | a6 |
| 2-4-234 | N-tolyl | N-tolyl | t-bu | t-bu | H | a6 |
| 2-4-235 | N-tolyl | N-tolyl | methyl | methyl | H | a6 |
| 2-4-236 | N-tolyl | N-tolyl | b1 | b1 | H | a6 |
| 2-4-237 | N-tolyl | N-tolyl | b2 | b2 | H | a6 |
| 2-4-238 | N-tolyl | N-tolyl | b3 | b3 | H | a6 |
| 2-4-239 | N-tolyl | N-tolyl | H | H | a1 | H |
| 2-4-240 | N-tolyl | N-tolyl | t-bu | t-bu | a1 | H |
| 2-4-241 | N-tolyl | N-tolyl | methyl | methyl | a1 | H |
| 2-4-242 | N-tolyl | N-tolyl | b1 | b1 | a1 | H |
| 2-4-243 | N-tolyl | N-tolyl | b2 | b2 | a1 | H |
| 2-4-244 | N-tolyl | N-tolyl | b3 | b3 | a1 | H |
| 2-4-245 | N-tolyl | N-tolyl | H | H | a2 | H |
| 2-4-246 | N-tolyl | N-tolyl | t-bu | t-bu | a2 | H |
| 2-4-247 | N-tolyl | N-tolyl | methyl | methyl | a2 | H |
| 2-4-248 | N-tolyl | N-tolyl | b1 | b1 | a2 | H |
| 2-4-249 | N-tolyl | N-tolyl | b2 | b2 | a2 | H |
| 2-4-250 | N-tolyl | N-tolyl | b3 | b3 | a2 | H |
| 2-4-251 | N-tolyl | N-tolyl | H | H | a3 | H |
| 2-4-252 | N-tolyl | N-tolyl | t-bu | t-bu | a3 | H |
| 2-4-253 | N-tolyl | N-tolyl | methyl | methyl | a3 | H |
| 2-4-254 | N-tolyl | N-tolyl | b1 | b1 | a3 | H |
| 2-4-255 | N-tolyl | N-tolyl | b2 | b2 | a3 | H |
| 2-4-256 | N-tolyl | N-tolyl | b3 | b3 | a3 | H |
| 2-4-257 | N-tolyl | N-tolyl | H | H | a4 | H |
| 2-4-258 | N-tolyl | N-tolyl | t-bu | t-bu | a4 | H |
| 2-4-259 | N-tolyl | N-tolyl | methyl | methyl | a4 | H |
| 2-4-260 | N-tolyl | N-tolyl | b1 | b1 | a4 | H |
| 2-4-261 | N-tolyl | N-tolyl | b2 | b2 | a4 | H |
| 2-4-262 | N-tolyl | N-tolyl | b3 | b3 | a4 | H |
| 2-4-263 | N-tolyl | N-tolyl | H | H | a5 | H |
| 2-4-264 | N-tolyl | N-tolyl | t-bu | t-bu | a5 | H |
| 2-4-265 | N-tolyl | N-tolyl | methyl | methyl | a5 | H |
| 2-4-266 | N-tolyl | N-tolyl | b1 | b1 | a5 | H |
| 2-4-267 | N-tolyl | N-tolyl | b2 | b2 | a5 | H |
| 2-4-268 | N-tolyl | N-tolyl | b3 | b3 | a5 | H |
| 2-4-269 | N-tolyl | N-tolyl | H | H | a6 | H |
| 2-4-270 | N-tolyl | N-tolyl | t-bu | t-bu | a6 | H |
| 2-4-271 | N-tolyl | N-tolyl | methyl | methyl | a6 | H |
| 2-4-272 | N-tolyl | N-tolyl | b1 | b1 | a6 | H |
| 2-4-273 | N-tolyl | N-tolyl | b2 | b2 | a6 | H |
| 2-4-274 | N-tolyl | N-tolyl | b3 | b3 | a6 | H |
| 2-4-275 | N-tolyl | N-tolyl | H | H | b1 | H |
| 2-4-276 | N-tolyl | N-tolyl | t-bu | t-bu | b1 | H |
| 2-4-277 | N-tolyl | N-tolyl | methyl | methyl | b1 | H |
| 2-4-278 | N-tolyl | N-tolyl | b1 | b1 | b1 | H |
| 2-4-279 | N-tolyl | N-tolyl | b2 | b2 | b1 | H |
| 2-4-280 | N-tolyl | N-tolyl | b3 | b3 | b1 | H |
| 2-4-281 | N-tolyl | N-tolyl | H | H | b2 | H |
| 2-4-282 | N-tolyl | N-tolyl | t-bu | t-bu | b2 | H |
| 2-4-283 | N-tolyl | N-tolyl | methyl | methyl | b2 | H |
| 2-4-284 | N-tolyl | N-tolyl | b1 | b1 | b2 | H |
| 2-4-285 | N-tolyl | N-tolyl | b2 | b2 | b2 | H |
| 2-4-286 | N-tolyl | N-tolyl | b3 | b3 | b2 | H |
| 2-4-287 | N-tolyl | N-tolyl | H | H | b3 | H |
| 2-4-288 | N-tolyl | N-tolyl | t-bu | t-bu | b3 | H |
| 2-4-289 | N-tolyl | N-tolyl | methyl | methyl | b3 | H |
| 2-4-290 | N-tolyl | N-tolyl | b1 | b1 | b3 | H |
| 2-4-291 | N-tolyl | N-tolyl | b2 | b2 | b3 | H |
| 2-4-292 | N-tolyl | N-tolyl | b3 | b3 | b3 | H |
| 2-4-293 | N-tolyl | N-tolyl | H | H | b4 | H |
| 2-4-294 | N-tolyl | N-tolyl | t-bu | t-bu | b4 | H |
| 2-4-295 | N-tolyl | N-tolyl | methyl | methyl | b4 | H |
| 2-4-296 | N-tolyl | N-tolyl | b1 | b1 | b4 | H |
| 2-4-297 | N-tolyl | N-tolyl | b2 | b2 | b4 | H |
| 2-4-298 | N-tolyl | N-tolyl | b3 | b3 | b4 | H |
| 2-4-299 | N-tolyl | N-tolyl | H | H | b5 | H |
| 2-4-300 | N-tolyl | N-tolyl | t-bu | t-bu | b5 | H |
| 2-4-301 | N-tolyl | N-tolyl | methyl | methyl | b5 | H |
| 2-4-302 | N-tolyl | N-tolyl | b1 | b1 | b5 | H |
| 2-4-303 | N-tolyl | N-tolyl | b2 | b2 | b5 | H |
| 2-4-304 | N-tolyl | N-tolyl | b3 | b3 | b5 | H |
| 2-4-305 | N-tolyl | N-tolyl | H | H | b6 | H |
| 2-4-306 | N-tolyl | N-tolyl | t-bu | t-bu | b6 | H |
| 2-4-307 | N-tolyl | N-tolyl | methyl | methyl | b6 | H |
| 2-4-308 | N-tolyl | N-tolyl | b1 | b1 | b6 | H |
| 2-4-309 | N-tolyl | N-tolyl | b2 | b2 | b6 | H |
| 2-4-310 | N-tolyl | N-tolyl | b3 | b3 | b6 | H |
| 2-4-311 | N-tolyl | N-tolyl | H | H | b7 | H |
| 2-4-312 | N-tolyl | N-tolyl | t-bu | t-bu | b7 | H |
| 2-4-313 | N-tolyl | N-tolyl | methyl | methyl | b7 | H |
| 2-4-314 | N-tolyl | N-tolyl | b1 | b1 | b7 | H |
| 2-4-315 | N-tolyl | N-tolyl | b2 | b2 | b7 | H |
| 2-4-316 | N-tolyl | N-tolyl | b3 | b3 | b7 | H |
| 2-4-317 | N-tolyl | N-tolyl | H | H | b8 | H |
| 2-4-318 | N-tolyl | N-tolyl | t-bu | t-bu | b8 | H |
| 2-4-319 | N-tolyl | N-tolyl | methyl | methyl | b8 | H |
| 2-4-320 | N-tolyl | N-tolyl | b1 | b1 | b8 | H |
| 2-4-321 | N-tolyl | N-tolyl | b2 | b2 | b8 | H |
| 2-4-322 | N-tolyl | N-tolyl | b3 | b3 | b8 | H |
| 2-4-323 | N-tolyl | N-tolyl | H | H | b9 | H |
| 2-4-324 | N-tolyl | N-tolyl | t-bu | t-bu | b9 | H |
| 2-4-325 | N-tolyl | N-tolyl | methyl | methyl | b9 | H |
| 2-4-326 | N-tolyl | N-tolyl | b1 | b1 | b9 | H |
| 2-4-327 | N-tolyl | N-tolyl | b2 | b2 | b9 | H |
| 2-4-328 | N-tolyl | N-tolyl | b3 | b3 | b9 | H |
| 2-4-329 | N-tolyl | N-tolyl | H | H | b10 | H |
| 2-4-330 | N-tolyl | N-tolyl | t-bu | t-bu | b10 | H |
| 2-4-331 | N-tolyl | N-tolyl | methyl | methyl | b10 | H |
| 2-4-332 | N-tolyl | N-tolyl | b1 | b1 | b10 | H |
| 2-4-333 | N-tolyl | N-tolyl | b2 | b2 | b10 | H |
| 2-4-334 | N-tolyl | N-tolyl | b3 | b3 | b10 | H |
| 2-4-335 | N-tolyl | N-tolyl | H | H | a1 | a1 |
| 2-4-336 | N-tolyl | N-tolyl | t-bu | t-bu | a1 | a1 |
| 2-4-337 | N-tolyl | N-tolyl | methyl | methyl | a1 | a1 |
| 2-4-338 | N-tolyl | N-tolyl | b1 | b1 | a1 | a1 |
| 2-4-339 | N-tolyl | N-tolyl | b2 | b2 | a1 | a1 |
| 2-4-340 | N-tolyl | N-tolyl | b3 | b3 | a1 | a1 |
| 2-4-341 | N-tolyl | N-tolyl | H | H | a2 | a2 |
| 2-4-342 | N-tolyl | N-tolyl | t-bu | t-bu | a2 | a2 |
| 2-4-343 | N-tolyl | N-tolyl | methyl | methyl | a2 | a2 |
| 2-4-344 | N-tolyl | N-tolyl | b1 | b1 | a2 | a2 |
| 2-4-345 | N-tolyl | N-tolyl | b2 | b2 | a2 | a2 |
| 2-4-346 | N-tolyl | N-tolyl | b3 | b3 | a2 | a2 |
| 2-4-347 | N-tolyl | N-tolyl | H | H | a3 | a3 |
| 2-4-348 | N-tolyl | N-tolyl | t-bu | t-bu | a3 | a3 |
| 2-4-349 | N-tolyl | N-tolyl | methyl | methyl | a3 | a3 |
| 2-4-350 | N-tolyl | N-tolyl | b1 | b1 | a3 | a3 |
| 2-4-351 | N-tolyl | N-tolyl | b2 | b2 | a3 | a3 |
| 2-4-352 | N-tolyl | N-tolyl | b3 | b3 | a3 | a3 |
| 2-4-353 | N-tolyl | N-tolyl | H | H | b1 | b1 |
| 2-4-354 | N-tolyl | N-tolyl | t-bu | t-bu | b1 | b1 |
| 2-4-355 | N-tolyl | N-tolyl | methyl | methyl | b1 | b1 |
| 2-4-356 | N-tolyl | N-tolyl | b1 | b1 | b1 | b1 |
| 2-4-357 | N-tolyl | N-tolyl | b2 | b2 | b1 | b1 |
| 2-4-358 | N-tolyl | N-tolyl | b3 | b3 | b1 | b1 |
| 2-4-359 | N-tolyl | N-tolyl | H | H | b2 | b2 |
| 2-4-360 | N-tolyl | N-tolyl | t-bu | t-bu | b2 | b2 |
| 2-4-361 | N-tolyl | N-tolyl | methyl | methyl | b2 | b2 |
| 2-4-362 | N-tolyl | N-tolyl | b1 | b1 | b2 | b2 |
| 2-4-363 | N-tolyl | N-tolyl | b2 | b2 | b2 | b2 |
| 2-4-364 | N-tolyl | N-tolyl | b3 | b3 | b2 | b2 |
| 2-4-365 | N-tolyl | N-tolyl | H | H | b3 | b3 |
| 2-4-366 | N-tolyl | N-tolyl | t-bu | t-bu | b3 | b3 |
| 2-4-367 | N-tolyl | N-tolyl | methyl | methyl | b3 | b3 |
| 2-4-368 | N-tolyl | N-tolyl | b1 | b1 | b3 | b3 |
| 2-4-369 | N-tolyl | N-tolyl | b2 | b2 | b3 | b3 |
| 2-4-370 | N-tolyl | N-tolyl | b3 | b3 | b3 | b3 |
| 2-4-371 | N-tolyl | N-tolyl | H | H | b5 | b5 |
| 2-4-372 | N-tolyl | N-tolyl | t-bu | t-bu | b5 | b5 |
| 2-4-373 | N-tolyl | N-tolyl | methyl | methyl | b5 | b5 |
| 2-4-374 | N-tolyl | N-tolyl | b1 | b1 | b5 | b5 |
| 2-4-375 | N-tolyl | N-tolyl | b2 | b2 | b5 | b5 |
| 2-4-376 | N-tolyl | N-tolyl | b3 | b3 | b5 | b5 |
| 2-4-377 | N-tolyl | N-tolyl | H | H | b6 | b6 |
| 2-4-378 | N-tolyl | N-tolyl | t-bu | t-bu | b6 | b6 |
| 2-4-379 | N-tolyl | N-tolyl | methyl | methyl | b6 | b6 |
| 2-4-380 | N-tolyl | N-tolyl | b1 | b1 | b6 | b6 |
| 2-4-381 | N-tolyl | N-tolyl | b2 | b2 | b6 | b6 |
| 2-4-382 | N-tolyl | N-tolyl | b3 | b3 | b6 | b6 |
| 2-4-383 | N-tolyl | N-tolyl | H | H | b7 | b7 |
| 2-4-384 | N-tolyl | N-tolyl | t-bu | t-bu | b7 | b7 |
| 2-4-385 | N-tolyl | N-tolyl | methyl | methyl | b7 | b7 |
| 2-4-386 | N-tolyl | N-tolyl | b1 | b1 | b7 | b7 |
| 2-4-387 | N-tolyl | N-tolyl | b2 | b2 | b7 | b7 |
| 2-4-388 | N-tolyl | N-tolyl | b3 | b3 | b7 | b7 |
| 2-4-389 | N-Biph | N-Biph | H | H | H | H |
| 2-4-390 | N-Biph | N-Biph | t-bu | t-bu | H | H |
| 2-4-391 | N-Biph | N-Biph | methyl | methyl | H | H |
| 2-4-392 | N-Biph | N-Biph | t-bu | t-bu | t-bu | t-bu |
| 2-4-393 | N-Biph | N-Biph | methyl | methyl | methyl | methyl |
| 2-4-394 | N-Biph | N-Biph | b1 | b1 | H | H |
| 2-4-395 | N-Biph | N-Biph | b2 | b2 | H | H |
| 2-4-396 | N-Biph | N-Biph | b3 | b3 | H | H |
| 2-4-397 | N-Biph | N-Biph | H | H | H | a1 |
| 2-4-398 | N-Biph | N-Biph | t-bu | t-bu | H | a1 |
| 2-4-399 | N-Biph | N-Biph | methyl | methyl | H | a1 |
| 2-4-400 | N-Biph | N-Biph | b1 | b1 | H | a1 |
| 2-4-401 | N-Biph | N-Biph | b2 | b2 | H | a1 |
| 2-4-402 | N-Biph | N-Biph | b3 | b3 | H | a1 |
| 2-4-403 | N-Biph | N-Biph | H | H | H | a2 |
| 2-4-404 | N-Biph | N-Biph | t-bu | t-bu | H | a2 |
| 2-4-405 | N-Biph | N-Biph | methyl | methyl | H | a2 |
| 2-4-406 | N-Biph | N-Biph | b1 | b1 | H | a2 |
| 2-4-407 | N-Biph | N-Biph | b2 | b2 | H | a2 |
| 2-4-408 | N-Biph | N-Biph | b3 | b3 | H | a2 |
| 2-4-409 | N-Biph | N-Biph | H | H | H | a3 |
| 2-4-410 | N-Biph | N-Biph | t-bu | t-bu | H | a3 |
| 2-4-411 | N-Biph | N-Biph | methyl | methyl | H | a3 |
| 2-4-412 | N-Biph | N-Biph | b1 | b1 | H | a3 |
| 2-4-413 | N-Biph | N-Biph | b2 | b2 | H | a3 |
| 2-4-414 | N-Biph | N-Biph | b3 | b3 | H | a3 |
| 2-4-415 | N-Biph | N-Biph | H | H | H | a4 |
| 2-4-416 | N-Biph | N-Biph | t-bu | t-bu | H | a4 |
| 2-4-417 | N-Biph | N-Biph | methyl | methyl | H | a4 |
| 2-4-418 | N-Biph | N-Biph | b1 | b1 | H | a4 |
| 2-4-419 | N-Biph | N-Biph | b2 | b2 | H | a4 |
| 2-4-420 | N-Biph | N-Biph | b3 | b3 | H | a4 |
| 2-4-421 | N-Biph | N-Biph | H | H | H | a5 |
| 2-4-422 | N-Biph | N-Biph | t-bu | t-bu | H | a5 |
| 2-4-423 | N-Biph | N-Biph | methyl | methyl | H | a5 |
| 2-4-424 | N-Biph | N-Biph | b1 | b1 | H | a5 |
| 2-4-425 | N-Biph | N-Biph | b2 | b2 | H | a5 |
| 2-4-426 | N-Biph | N-Biph | b3 | b3 | H | a5 |
| 2-4-427 | N-Biph | N-Biph | H | H | H | a6 |
| 2-4-428 | N-Biph | N-Biph | t-bu | t-bu | H | a6 |
| 2-4-429 | N-Biph | N-Biph | methyl | methyl | H | a6 |
| 2-4-430 | N-Biph | N-Biph | b1 | b1 | H | a6 |
| 2-4-431 | N-Biph | N-Biph | b2 | b2 | H | a6 |
| 2-4-432 | N-Biph | N-Biph | b3 | b3 | H | a6 |
| 2-4-433 | N-Biph | N-Biph | H | H | a1 | H |
| 2-4-434 | N-Biph | N-Biph | t-bu | t-bu | a1 | H |
| 2-4-435 | N-Biph | N-Biph | methyl | methyl | a1 | H |
| 2-4-436 | N-Biph | N-Biph | b1 | b1 | a1 | H |
| 2-4-437 | N-Biph | N-Biph | b2 | b2 | a1 | H |
| 2-4-438 | N-Biph | N-Biph | b3 | b3 | a1 | H |
| 2-4-439 | N-Biph | N-Biph | H | H | a2 | H |
| 2-4-440 | N-Biph | N-Biph | t-bu | t-bu | a2 | H |
| 2-4-441 | N-Biph | N-Biph | methyl | methyl | a2 | H |
| 2-4-442 | N-Biph | N-Biph | b1 | b1 | a2 | H |
| 2-4-443 | N-Biph | N-Biph | b2 | b2 | a2 | H |
| 2-4-444 | N-Biph | N-Biph | b3 | b3 | a2 | H |
| 2-4-445 | N-Biph | N-Biph | H | H | a3 | H |
| 2-4-446 | N-Biph | N-Biph | t-bu | t-bu | a3 | H |
| 2-4-447 | N-Biph | N-Biph | methyl | methyl | a3 | H |
| 2-4-448 | N-Biph | N-Biph | b1 | b1 | a3 | H |
| 2-4-449 | N-Biph | N-Biph | b2 | b2 | a3 | H |
| 2-4-450 | N-Biph | N-Biph | b3 | b3 | a3 | H |
| 2-4-451 | N-Biph | N-Biph | H | H | a4 | H |
| 2-4-452 | N-Biph | N-Biph | t-bu | t-bu | a4 | H |
| 2-4-453 | N-Biph | N-Biph | methyl | methyl | a4 | H |
| 2-4-454 | N-Biph | N-Biph | b1 | b1 | a4 | H |
| 2-4-455 | N-Biph | N-Biph | b2 | b2 | a4 | H |
| 2-4-456 | N-Biph | N-Biph | b3 | b3 | a4 | H |
| 2-4-457 | N-Biph | N-Biph | H | H | a5 | H |
| 2-4-458 | N-Biph | N-Biph | t-bu | t-bu | a5 | H |
| 2-4-459 | N-Biph | N-Biph | methyl | methyl | a5 | H |
| 2-4-460 | N-Biph | N-Biph | b1 | b1 | a5 | H |
| 2-4-461 | N-Biph | N-Biph | b2 | b2 | a5 | H |
| 2-4-462 | N-Biph | N-Biph | b3 | b3 | a5 | H |
| 2-4-463 | N-Biph | N-Biph | H | H | a6 | H |
| 2-4-464 | N-Biph | N-Biph | t-bu | t-bu | a6 | H |
| 2-4-465 | N-Biph | N-Biph | methyl | methyl | a6 | H |
| 2-4-466 | N-Biph | N-Biph | b1 | b1 | a6 | H |
| 2-4-467 | N-Biph | N-Biph | b2 | b2 | a6 | H |
| 2-4-468 | N-Biph | N-Biph | b3 | b3 | a6 | H |
| 2-4-469 | N-Biph | N-Biph | H | H | b1 | H |
| 2-4-470 | N-Biph | N-Biph | t-bu | t-bu | b1 | H |
| 2-4-471 | N-Biph | N-Biph | methyl | methyl | b1 | H |
| 2-4-472 | N-Biph | N-Biph | b1 | b1 | b1 | H |
| 2-4-473 | N-Biph | N-Biph | b2 | b2 | b1 | H |
| 2-4-474 | N-Biph | N-Biph | b3 | b3 | b1 | H |
| 2-4-475 | N-Biph | N-Biph | H | H | b2 | H |
| 2-4-476 | N-Biph | N-Biph | t-bu | t-bu | b2 | H |
| 2-4-477 | N-Biph | N-Biph | methyl | methyl | b2 | H |
| 2-4-478 | N-Biph | N-Biph | b1 | b1 | b2 | H |
| 2-4-479 | N-Biph | N-Biph | b2 | b2 | b2 | H |
| 2-4-480 | N-Biph | N-Biph | b3 | b3 | b2 | H |
| 2-4-481 | N-Biph | N-Biph | H | H | b3 | H |
| 2-4-482 | N-Biph | N-Biph | t-bu | t-bu | b3 | H |
| 2-4-483 | N-Biph | N-Biph | methyl | methyl | b3 | H |
| 2-4-484 | N-Biph | N-Biph | b1 | b1 | b3 | H |
| 2-4-485 | N-Biph | N-Biph | b2 | b2 | b3 | H |
| 2-4-486 | N-Biph | N-Biph | b3 | b3 | b3 | H |
| 2-4-487 | N-Biph | N-Biph | H | H | b4 | H |
| 2-4-488 | N-Biph | N-Biph | t-bu | t-bu | b4 | H |
| 2-4-489 | N-Biph | N-Biph | methyl | methyl | b4 | H |
| 2-4-490 | N-Biph | N-Biph | b1 | b1 | b4 | H |
| 2-4-491 | N-Biph | N-Biph | b2 | b2 | b4 | H |
| 2-4-492 | N-Biph | N-Biph | b3 | b3 | b4 | H |
| 2-4-493 | N-Biph | N-Biph | H | H | b5 | H |
| 2-4-494 | N-Biph | N-Biph | t-bu | t-bu | b5 | H |
| 2-4-495 | N-Biph | N-Biph | methyl | methyl | b5 | H |
| 2-4-496 | N-Biph | N-Biph | b1 | b1 | b5 | H |
| 2-4-497 | N-Biph | N-Biph | b2 | b2 | b5 | H |
| 2-4-498 | N-Biph | N-Biph | b3 | b3 | b5 | H |
| 2-4-499 | N-Biph | N-Biph | H | H | b6 | H |
| 2-4-500 | N-Biph | N-Biph | t-bu | t-bu | b6 | H |
| 2-4-501 | N-Biph | N-Biph | methyl | methyl | b6 | H |
| 2-4-502 | N-Biph | N-Biph | b1 | b1 | b6 | H |
| 2-4-503 | N-Biph | N-Biph | b2 | b2 | b6 | H |
| 2-4-504 | N-Biph | N-Biph | b3 | b3 | b6 | H |
| 2-4-505 | N-Biph | N-Biph | H | H | b7 | H |
| 2-4-506 | N-Biph | N-Biph | t-bu | t-bu | b7 | H |
| 2-4-507 | N-Biph | N-Biph | methyl | methyl | b7 | H |
| 2-4-508 | N-Biph | N-Biph | b1 | b1 | b7 | H |
| 2-4-509 | N-Biph | N-Biph | b2 | b2 | b7 | H |
| 2-4-510 | N-Biph | N-Biph | b3 | b3 | b7 | H |
| 2-4-511 | N-Biph | N-Biph | H | H | b8 | H |
| 2-4-512 | N-Biph | N-Biph | t-bu | t-bu | b8 | H |
| 2-4-513 | N-Biph | N-Biph | methyl | methyl | b8 | H |
| 2-4-514 | N-Biph | N-Biph | b1 | b1 | b8 | H |
| 2-4-515 | N-Biph | N-Biph | b2 | b2 | b8 | H |
| 2-4-516 | N-Biph | N-Biph | b3 | b3 | b8 | H |
| 2-4-517 | N-Biph | N-Biph | H | H | b9 | H |
| 2-4-518 | N-Biph | N-Biph | t-bu | t-bu | b9 | H |
| 2-4-519 | N-Biph | N-Biph | methyl | methyl | b9 | H |
| 2-4-520 | N-Biph | N-Biph | b1 | b1 | b9 | H |
| 2-4-521 | N-Biph | N-Biph | b2 | b2 | b9 | H |
| 2-4-522 | N-Biph | N-Biph | b3 | b3 | b9 | H |
| 2-4-523 | N-Biph | N-Biph | H | H | b10 | H |
| 2-4-524 | N-Biph | N-Biph | t-bu | t-bu | b10 | H |
| 2-4-525 | N-Biph | N-Biph | methyl | methyl | b10 | H |
| 2-4-526 | N-Biph | N-Biph | b1 | b1 | b10 | H |
| 2-4-527 | N-Biph | N-Biph | b2 | b2 | b10 | H |
| 2-4-528 | N-Biph | N-Biph | b3 | b3 | b10 | H |
| 2-4-529 | N-Biph | N-Biph | H | H | a1 | a1 |
| 2-4-530 | N-Biph | N-Biph | t-bu | t-bu | a1 | a1 |
| 2-4-531 | N-Biph | N-Biph | methyl | methyl | a1 | a1 |
| 2-4-532 | N-Biph | N-Biph | b1 | b1 | a1 | a1 |
| 2-4-533 | N-Biph | N-Biph | b2 | b2 | a1 | a1 |
| 2-4-534 | N-Biph | N-Biph | b3 | b3 | a1 | a1 |
| 2-4-535 | N-Biph | N-Biph | H | H | a2 | a2 |
| 2-4-536 | N-Biph | N-Biph | t-bu | t-bu | a2 | a2 |
| 2-4-537 | N-Biph | N-Biph | methyl | methyl | a2 | a2 |
| 2-4-538 | N-Biph | N-Biph | b1 | b1 | a2 | a2 |
| 2-4-539 | N-Biph | N-Biph | b2 | b2 | a2 | a2 |
| 2-4-540 | N-Biph | N-Biph | b3 | b3 | a2 | a2 |
| 2-4-541 | N-Biph | N-Biph | H | H | a3 | a3 |
| 2-4-542 | N-Biph | N-Biph | t-bu | t-bu | a3 | a3 |
| 2-4-543 | N-Biph | N-Biph | methyl | methyl | a3 | a3 |
| 2-4-544 | N-Biph | N-Biph | b1 | b1 | a3 | a3 |
| 2-4-545 | N-Biph | N-Biph | b2 | b2 | a3 | a3 |
| 2-4-546 | N-Biph | N-Biph | b3 | b3 | a3 | a3 |
| 2-4-547 | N-Biph | N-Biph | H | H | b1 | b1 |
| 2-4-548 | N-Biph | N-Biph | t-bu | t-bu | b1 | b1 |
| 2-4-549 | N-Biph | N-Biph | methyl | methyl | b1 | b1 |
| 2-4-550 | N-Biph | N-Biph | b1 | b1 | b1 | b1 |
| 2-4-551 | N-Biph | N-Biph | b2 | b2 | b1 | b1 |
| 2-4-552 | N-Biph | N-Biph | b3 | b3 | b1 | b1 |
| 2-4-553 | N-Biph | N-Biph | H | H | b2 | b2 |
| 2-4-554 | N-Biph | N-Biph | t-bu | t-bu | b2 | b2 |
| 2-4-555 | N-Biph | N-Biph | methyl | methyl | b2 | b2 |
| 2-4-556 | N-Biph | N-Biph | b1 | b1 | b2 | b2 |
| 2-4-557 | N-Biph | N-Biph | b2 | b2 | b2 | b2 |
| 2-4-558 | N-Biph | N-Biph | b3 | b3 | b2 | b2 |
| 2-4-559 | N-Biph | N-Biph | H | H | b3 | b3 |
| 2-4-560 | N-Biph | N-Biph | t-bu | t-bu | b3 | b3 |
| 2-4-561 | N-Biph | N-Biph | methyl | methyl | b3 | b3 |
| 2-4-562 | N-Biph | N-Biph | b1 | b1 | b3 | b3 |
| 2-4-563 | N-Biph | N-Biph | b2 | b2 | b3 | b3 |
| 2-4-564 | N-Biph | N-Biph | b3 | b3 | b3 | b3 |
| 2-4-565 | N-Biph | N-Biph | H | H | b5 | b5 |
| 2-4-566 | N-Biph | N-Biph | t-bu | t-bu | b5 | b5 |
| 2-4-567 | N-Biph | N-Biph | methyl | methyl | b5 | b5 |
| 2-4-568 | N-Biph | N-Biph | b1 | b1 | b5 | b5 |
| 2-4-569 | N-Biph | N-Biph | b2 | b2 | b5 | b5 |
| 2-4-570 | N-Biph | N-Biph | b3 | b3 | b5 | b5 |
| 2-4-571 | N-Biph | N-Biph | H | H | b6 | b6 |
| 2-4-572 | N-Biph | N-Biph | t-bu | t-bu | b6 | b6 |
| 2-4-573 | N-Biph | N-Biph | methyl | methyl | b6 | b6 |
| 2-4-574 | N-Biph | N-Biph | b1 | b1 | b6 | b6 |
| 2-4-575 | N-Biph | N-Biph | b2 | b2 | b6 | b6 |
| 2-4-576 | N-Biph | N-Biph | b3 | b3 | b6 | b6 |
| 2-4-577 | N-Biph | N-Biph | H | H | b7 | b7 |
| 2-4-578 | N-Biph | N-Biph | t-bu | t-bu | b7 | b7 |
| 2-4-579 | N-Biph | N-Biph | methyl | methyl | b7 | b7 |
| 2-4-580 | N-Biph | N-Biph | b1 | b1 | b7 | b7 |
| 2-4-581 | N-Biph | N-Biph | b2 | b2 | b7 | b7 |
| 2-4-582 | N-Biph | N-Biph | b3 | b3 | b7 | b7 |
| 2-4-583 | O | N—Ph | H | H | H | H |
| 2-4-584 | O | N—Ph | t-bu | t-bu | H | H |
| 2-4-585 | O | N—Ph | methyl | methyl | H | H |
| 2-4-586 | O | N—Ph | t-bu | t-bu | t-bu | t-bu |
| 2-4-587 | O | N—Ph | methyl | methyl | methyl | methyl |
| 2-4-588 | O | N—Ph | b1 | b1 | H | H |
| 2-4-589 | O | N—Ph | b2 | b2 | H | H |
| 2-4-590 | O | N—Ph | b3 | b3 | H | H |
| 2-4-591 | O | N—Ph | H | H | H | a1 |
| 2-4-592 | O | N—Ph | t-bu | t-bu | H | a1 |
| 2-4-593 | O | N—Ph | methyl | methyl | H | a1 |
| 2-4-594 | O | N—Ph | b1 | b1 | H | a1 |
| 2-4-595 | O | N—Ph | b2 | b2 | H | a1 |
| 2-4-596 | O | N—Ph | b3 | b3 | H | a1 |
| 2-4-597 | O | N—Ph | H | H | H | a2 |
| 2-4-598 | O | N—Ph | t-bu | t-bu | H | a2 |
| 2-4-599 | O | N—Ph | methyl | methyl | H | a2 |
| 2-4-600 | O | N—Ph | b1 | b1 | H | a2 |
| 2-4-601 | O | N—Ph | b2 | b2 | H | a2 |
| 2-4-602 | O | N—Ph | b3 | b3 | H | a2 |
| 2-4-603 | O | N—Ph | H | H | H | a3 |
| 2-4-604 | O | N—Ph | t-bu | t-bu | H | a3 |
| 2-4-605 | O | N—Ph | methyl | methyl | H | a3 |
| 2-4-606 | O | N—Ph | b1 | b1 | H | a3 |
| 2-4-607 | O | N—Ph | b2 | b2 | H | a3 |
| 2-4-608 | O | N—Ph | b3 | b3 | H | a3 |
| 2-4-609 | O | N—Ph | H | H | H | a4 |
| 2-4-610 | O | N—Ph | t-bu | t-bu | H | a4 |
| 2-4-611 | O | N—Ph | methyl | methyl | H | a4 |
| 2-4-612 | O | N—Ph | b1 | b1 | H | a4 |
| 2-4-613 | O | N—Ph | b2 | b2 | H | a4 |
| 2-4-614 | O | N—Ph | b3 | b3 | H | a4 |
| 2-4-615 | O | N—Ph | H | H | H | a5 |
| 2-4-616 | O | N—Ph | t-bu | t-bu | H | a5 |
| 2-4-617 | O | N—Ph | methyl | methyl | H | a5 |
| 2-4-618 | O | N—Ph | b1 | b1 | H | a5 |
| 2-4-619 | O | N—Ph | b2 | b2 | H | a5 |
| 2-4-620 | O | N—Ph | b3 | b3 | H | a5 |
| 2-4-621 | O | N—Ph | H | H | H | a6 |
| 2-4-622 | O | N—Ph | t-bu | t-bu | H | a6 |
| 2-4-623 | O | N—Ph | methyl | methyl | H | a6 |
| 2-4-624 | O | N—Ph | b1 | b1 | H | a6 |
| 2-4-625 | O | N—Ph | b2 | b2 | H | a6 |
| 2-4-626 | O | N—Ph | b3 | b3 | H | a6 |
| 2-4-627 | O | N—Ph | H | H | a1 | H |
| 2-4-628 | O | N—Ph | t-bu | t-bu | a1 | H |
| 2-4-629 | O | N—Ph | methyl | methyl | a1 | H |
| 2-4-630 | O | N—Ph | b1 | b1 | a1 | H |
| 2-4-631 | O | N—Ph | b2 | b2 | a1 | H |
| 2-4-632 | O | N—Ph | b3 | b3 | a1 | H |
| 2-4-633 | O | N—Ph | H | H | a2 | H |
| 2-4-634 | O | N—Ph | t-bu | t-bu | a2 | H |
| 2-4-635 | O | N—Ph | methyl | methyl | a2 | H |
| 2-4-636 | O | N—Ph | b1 | b1 | a2 | H |
| 2-4-637 | O | N—Ph | b2 | b2 | a2 | H |
| 2-4-638 | O | N—Ph | b3 | b3 | a2 | H |
| 2-4-639 | O | N—Ph | H | H | a3 | H |
| 2-4-640 | O | N—Ph | t-bu | t-bu | a3 | H |
| 2-4-641 | O | N—Ph | methyl | methyl | a3 | H |
| 2-4-642 | O | N—Ph | b1 | b1 | a3 | H |
| 2-4-643 | O | N—Ph | b2 | b2 | a3 | H |
| 2-4-644 | O | N—Ph | b3 | b3 | a3 | H |
| 2-4-645 | O | N—Ph | H | H | a4 | H |
| 2-4-646 | O | N—Ph | t-bu | t-bu | a4 | H |
| 2-4-647 | O | N—Ph | methyl | methyl | a4 | H |
| 2-4-648 | O | N—Ph | b1 | b1 | a4 | H |
| 2-4-649 | O | N—Ph | b2 | b2 | a4 | H |
| 2-4-650 | O | N—Ph | b3 | b3 | a4 | H |
| 2-4-651 | O | N—Ph | H | H | a5 | H |
| 2-4-652 | O | N—Ph | t-bu | t-bu | a5 | H |
| 2-4-653 | O | N—Ph | methyl | methyl | a5 | H |
| 2-4-654 | O | N—Ph | b1 | b1 | a5 | H |
| 2-4-655 | O | N—Ph | b2 | b2 | a5 | H |
| 2-4-656 | O | N—Ph | b3 | b3 | a5 | H |
| 2-4-657 | O | N—Ph | H | H | a6 | H |
| 2-4-658 | O | N—Ph | t-bu | t-bu | a6 | H |
| 2-4-659 | O | N—Ph | methyl | methyl | a6 | H |
| 2-4-660 | O | N—Ph | b1 | b1 | a6 | H |
| 2-4-661 | O | N—Ph | b2 | b2 | a6 | H |
| 2-4-662 | O | N—Ph | b3 | b3 | a6 | H |
| 2-4-663 | O | N—Ph | H | H | b1 | H |
| 2-4-664 | O | N—Ph | t-bu | t-bu | b1 | H |
| 2-4-665 | O | N—Ph | methyl | methyl | b1 | H |
| 2-4-666 | O | N—Ph | b1 | b1 | b1 | H |
| 2-4-667 | O | N—Ph | b2 | b2 | b1 | H |
| 2-4-668 | O | N—Ph | b3 | b3 | b1 | H |
| 2-4-669 | O | N—Ph | H | H | b2 | H |
| 2-4-670 | O | N—Ph | t-bu | t-bu | b2 | H |
| 2-4-671 | O | N—Ph | methyl | methyl | b2 | H |
| 2-4-672 | O | N—Ph | b1 | b1 | b2 | H |
| 2-4-673 | O | N—Ph | b2 | b2 | b2 | H |
| 2-4-674 | O | N—Ph | b3 | b3 | b2 | H |
| 2-4-675 | O | N—Ph | H | H | b3 | H |
| 2-4-676 | O | N—Ph | t-bu | t-bu | b3 | H |
| 2-4-677 | O | N—Ph | methyl | methyl | b3 | H |
| 2-4-678 | O | N—Ph | b1 | b1 | b3 | H |
| 2-4-679 | O | N—Ph | b2 | b2 | b3 | H |
| 2-4-680 | O | N—Ph | b3 | b3 | b3 | H |
| 2-4-681 | O | N—Ph | H | H | b4 | H |
| 2-4-682 | O | N—Ph | t-bu | t-bu | b4 | H |
| 2-4-683 | O | N—Ph | methyl | methyl | b4 | H |
| 2-4-684 | O | N—Ph | b1 | b1 | b4 | H |
| 2-4-685 | O | N—Ph | b2 | b2 | b4 | H |
| 2-4-686 | O | N—Ph | b3 | b3 | b4 | H |
| 2-4-687 | O | N—Ph | H | H | b5 | H |
| 2-4-688 | O | N—Ph | t-bu | t-bu | b5 | H |
| 2-4-689 | O | N—Ph | methyl | methyl | b5 | H |
| 2-4-690 | O | N—Ph | b1 | b1 | b5 | H |
| 2-4-691 | O | N—Ph | b2 | b2 | b5 | H |
| 2-4-692 | O | N—Ph | b3 | b3 | b5 | H |
| 2-4-693 | O | N—Ph | H | H | b6 | H |
| 2-4-694 | O | N—Ph | t-bu | t-bu | b6 | H |
| 2-4-695 | O | N—Ph | methyl | methyl | b6 | H |
| 2-4-696 | O | N—Ph | b1 | b1 | b6 | H |
| 2-4-697 | O | N—Ph | b2 | b2 | b6 | H |
| 2-4-698 | O | N—Ph | b3 | b3 | b6 | H |
| 2-4-699 | O | N—Ph | H | H | b7 | H |
| 2-4-700 | O | N—Ph | t-bu | t-bu | b7 | H |
| 2-4-701 | O | N—Ph | methyl | methyl | b7 | H |
| 2-4-702 | O | N—Ph | b1 | b1 | b7 | H |
| 2-4-703 | O | N—Ph | b2 | b2 | b7 | H |
| 2-4-704 | O | N—Ph | b3 | b3 | b7 | H |
| 2-4-705 | O | N—Ph | H | H | b8 | H |
| 2-4-706 | O | N—Ph | t-bu | t-bu | b8 | H |
| 2-4-707 | O | N—Ph | methyl | methyl | b8 | H |
| 2-4-708 | O | N—Ph | b1 | b1 | b8 | H |
| 2-4-709 | O | N—Ph | b2 | b2 | b8 | H |
| 2-4-710 | O | N—Ph | b3 | b3 | b8 | H |
| 2-4-711 | O | N—Ph | H | H | b9 | H |
| 2-4-712 | O | N—Ph | t-bu | t-bu | b9 | H |
| 2-4-713 | O | N—Ph | methyl | methyl | b9 | H |
| 2-4-714 | O | N—Ph | b1 | b1 | b9 | H |
| 2-4-715 | O | N—Ph | b2 | b2 | b9 | H |
| 2-4-716 | O | N—Ph | b3 | b3 | b9 | H |
| 2-4-717 | O | N—Ph | H | H | b10 | H |
| 2-4-718 | O | N—Ph | t-bu | t-bu | b10 | H |
| 2-4-719 | O | N—Ph | methyl | methyl | b10 | H |
| 2-4-720 | O | N—Ph | b1 | b1 | b10 | H |
| 2-4-721 | O | N—Ph | b2 | b2 | b10 | H |
| 2-4-722 | O | N—Ph | b3 | b3 | b10 | H |
| 2-4-723 | O | N—Ph | H | H | a1 | a1 |
| 2-4-724 | O | N—Ph | t-bu | t-bu | a1 | a1 |
| 2-4-725 | O | N—Ph | methyl | methyl | a1 | a1 |
| 2-4-726 | O | N—Ph | b1 | b1 | a1 | a1 |
| 2-4-727 | O | N—Ph | b2 | b2 | a1 | a1 |
| 2-4-728 | O | N—Ph | b3 | b3 | a1 | a1 |
| 2-4-729 | O | N—Ph | H | H | a2 | a2 |
| 2-4-730 | O | N—Ph | t-bu | t-bu | a2 | a2 |
| 2-4-731 | O | N—Ph | methyl | methyl | a2 | a2 |
| 2-4-732 | O | N—Ph | b1 | b1 | a2 | a2 |
| 2-4-733 | O | N—Ph | b2 | b2 | a2 | a2 |
| 2-4-734 | O | N—Ph | b3 | b3 | a2 | a2 |
| 2-4-735 | O | N—Ph | H | H | a3 | a3 |
| 2-4-736 | O | N—Ph | t-bu | t-bu | a3 | a3 |
| 2-4-737 | O | N—Ph | methyl | methyl | a3 | a3 |
| 2-4-738 | O | N—Ph | b1 | b1 | a3 | a3 |
| 2-4-739 | O | N—Ph | b2 | b2 | a3 | a3 |
| 2-4-740 | O | N—Ph | b3 | b3 | a3 | a3 |
| 2-4-741 | O | N—Ph | H | H | b1 | b1 |
| 2-4-742 | O | N—Ph | t-bu | t-bu | b1 | b1 |
| 2-4-743 | O | N—Ph | methyl | methyl | b1 | b1 |
| 2-4-744 | O | N—Ph | b1 | b1 | b1 | b1 |
| 2-4-745 | O | N—Ph | b2 | b2 | b1 | b1 |
| 2-4-746 | O | N—Ph | b3 | b3 | b1 | b1 |
| 2-4-747 | O | N—Ph | H | H | b2 | b2 |
| 2-4-748 | O | N—Ph | t-bu | t-bu | b2 | b2 |
| 2-4-749 | O | N—Ph | methyl | methyl | b2 | b2 |
| 2-4-750 | O | N—Ph | b1 | b1 | b2 | b2 |
| 2-4-751 | O | N—Ph | b2 | b2 | b2 | b2 |
| 2-4-752 | O | N—Ph | b3 | b3 | b2 | b2 |
| 2-4-753 | O | N—Ph | H | H | b3 | b3 |
| 2-4-754 | O | N—Ph | t-bu | t-bu | b3 | b3 |
| 2-4-755 | O | N—Ph | methyl | methyl | b3 | b3 |
| 2-4-756 | O | N—Ph | b1 | b1 | b3 | b3 |
| 2-4-757 | O | N—Ph | b2 | b2 | b3 | b3 |
| 2-4-758 | O | N—Ph | b3 | b3 | b3 | b3 |
| 2-4-759 | O | N—Ph | H | H | b5 | b5 |
| 2-4-760 | O | N—Ph | t-bu | t-bu | b5 | b5 |
| 2-4-761 | O | N—Ph | methyl | methyl | b5 | b5 |
| 2-4-762 | O | N—Ph | b1 | b1 | b5 | b5 |
| 2-4-763 | O | N—Ph | b2 | b2 | b5 | b5 |
| 2-4-764 | O | N—Ph | b3 | b3 | b5 | b5 |
| 2-4-765 | O | N—Ph | H | H | b6 | b6 |
| 2-4-766 | O | N—Ph | t-bu | t-bu | b6 | b6 |
| 2-4-767 | O | N—Ph | methyl | methyl | b6 | b6 |
| 2-4-768 | O | N—Ph | b1 | b1 | b6 | b6 |
| 2-4-769 | O | N—Ph | b2 | b2 | b6 | b6 |
| 2-4-770 | O | N—Ph | b3 | b3 | b6 | b6 |
| 2-4-771 | O | N—Ph | H | H | b7 | b7 |
| 2-4-772 | O | N—Ph | t-bu | t-bu | b7 | b7 |
| 2-4-773 | O | N—Ph | methyl | methyl | b7 | b7 |
| 2-4-774 | O | N—Ph | b1 | b1 | b7 | b7 |
| 2-4-775 | O | N—Ph | b2 | b2 | b7 | b7 |
| 2-4-776 | O | N—Ph | b3 | b3 | b7 | b7 |
| 2-4-777 | S | N—Ph | H | H | H | H |
| 2-4-778 | S | N—Ph | t-bu | t-bu | H | H |
| 2-4-779 | S | N—Ph | methyl | methyl | H | H |
| 2-4-780 | S | N—Ph | t-bu | t-bu | t-bu | t-bu |
| 2-4-781 | S | N—Ph | methyl | methyl | methyl | methyl |
| 2-4-782 | S | N—Ph | b1 | b1 | H | H |
| 2-4-783 | S | N—Ph | b2 | b2 | H | H |
| 2-4-784 | S | N—Ph | b3 | b3 | H | H |
| 2-4-785 | S | N—Ph | H | H | H | a1 |
| 2-4-786 | S | N—Ph | t-bu | t-bu | H | a1 |
| 2-4-787 | S | N—Ph | methyl | methyl | H | a1 |
| 2-4-788 | S | N—Ph | b1 | b1 | H | a1 |
| 2-4-789 | S | N—Ph | b2 | b2 | H | a1 |
| 2-4-790 | S | N—Ph | b3 | b3 | H | a1 |
| 2-4-791 | S | N—Ph | H | H | H | a2 |
| 2-4-792 | S | N—Ph | t-bu | t-bu | H | a2 |
| 2-4-793 | S | N—Ph | methyl | methyl | H | a2 |
| 2-4-794 | S | N—Ph | b1 | b1 | H | a2 |
| 2-4-795 | S | N—Ph | b2 | b2 | H | a2 |
| 2-4-796 | S | N—Ph | b3 | b3 | H | a2 |
| 2-4-797 | S | N—Ph | H | H | H | a3 |
| 2-4-798 | S | N—Ph | t-bu | t-bu | H | a3 |
| 2-4-799 | S | N—Ph | methyl | methyl | H | a3 |
| 2-4-800 | S | N—Ph | b1 | b1 | H | a3 |
| 2-4-801 | S | N—Ph | b2 | b2 | H | a3 |
| 2-4-802 | S | N—Ph | b3 | b3 | H | a3 |
| 2-4-803 | S | N—Ph | H | H | H | a4 |
| 2-4-804 | S | N—Ph | t-bu | t-bu | H | a4 |
| 2-4-805 | S | N—Ph | methyl | methyl | H | a4 |
| 2-4-806 | S | N—Ph | b1 | b1 | H | a4 |
| 2-4-807 | S | N—Ph | b2 | b2 | H | a4 |
| 2-4-808 | S | N—Ph | b3 | b3 | H | a4 |
| 2-4-809 | S | N—Ph | H | H | H | a5 |
| 2-4-810 | S | N—Ph | t-bu | t-bu | H | a5 |
| 2-4-811 | S | N—Ph | methyl | methyl | H | a5 |
| 2-4-812 | S | N—Ph | b1 | b1 | H | a5 |
| 2-4-813 | S | N—Ph | b2 | b2 | H | a5 |
| 2-4-814 | S | N—Ph | b3 | b3 | H | a5 |
| 2-4-815 | S | N—Ph | H | H | H | a6 |
| 2-4-816 | S | N—Ph | t-bu | t-bu | H | a6 |
| 2-4-817 | S | N—Ph | methyl | methyl | H | a6 |
| 2-4-818 | S | N—Ph | b1 | b1 | H | a6 |
| 2-4-819 | S | N—Ph | b2 | b2 | H | a6 |
| 2-4-820 | S | N—Ph | b3 | b3 | H | a6 |
| 2-4-821 | S | N—Ph | H | H | a1 | H |
| 2-4-822 | S | N—Ph | t-bu | t-bu | a1 | H |
| 2-4-823 | S | N—Ph | methyl | methyl | a1 | H |
| 2-4-824 | S | N—Ph | b1 | b1 | a1 | H |
| 2-4-825 | S | N—Ph | b2 | b2 | a1 | H |
| 2-4-826 | S | N—Ph | b3 | b3 | a1 | H |
| 2-4-827 | S | N—Ph | H | H | a2 | H |
| 2-4-828 | S | N—Ph | t-bu | t-bu | a2 | H |
| 2-4-829 | S | N—Ph | methyl | methyl | a2 | H |
| 2-4-830 | S | N—Ph | b1 | b1 | a2 | H |
| 2-4-831 | S | N—Ph | b2 | b2 | a2 | H |
| 2-4-832 | S | N—Ph | b3 | b3 | a2 | H |
| 2-4-833 | S | N—Ph | H | H | a3 | H |
| 2-4-834 | S | N—Ph | t-bu | t-bu | a3 | H |
| 2-4-835 | S | N—Ph | methyl | methyl | a3 | H |
| 2-4-836 | S | N—Ph | b1 | b1 | a3 | H |
| 2-4-837 | S | N—Ph | b2 | b2 | a3 | H |
| 2-4-838 | S | N—Ph | b3 | b3 | a3 | H |
| 2-4-839 | S | N—Ph | H | H | a4 | H |
| 2-4-840 | S | N—Ph | t-bu | t-bu | a4 | H |
| 2-4-841 | S | N—Ph | methyl | methyl | a4 | H |
| 2-4-842 | S | N—Ph | b1 | b1 | a4 | H |
| 2-4-843 | S | N—Ph | b2 | b2 | a4 | H |
| 2-4-844 | S | N—Ph | b3 | b3 | a4 | H |
| 2-4-845 | S | N—Ph | H | H | a5 | H |
| 2-4-846 | S | N—Ph | t-bu | t-bu | a5 | H |
| 2-4-847 | S | N—Ph | methyl | methyl | a5 | H |
| 2-4-848 | S | N—Ph | b1 | b1 | a5 | H |
| 2-4-849 | S | N—Ph | b2 | b2 | a5 | H |
| 2-4-850 | S | N—Ph | b3 | b3 | a5 | H |
| 2-4-851 | S | N—Ph | H | H | a6 | H |
| 2-4-852 | S | N—Ph | t-bu | t-bu | a6 | H |
| 2-4-853 | S | N—Ph | methyl | methyl | a6 | H |
| 2-4-854 | S | N—Ph | b1 | b1 | a6 | H |
| 2-4-855 | S | N—Ph | b2 | b2 | a6 | H |
| 2-4-856 | S | N—Ph | b3 | b3 | a6 | H |
| 2-4-857 | S | N—Ph | H | H | b1 | H |
| 2-4-858 | S | N—Ph | t-bu | t-bu | b1 | H |
| 2-4-859 | S | N—Ph | methyl | methyl | b1 | H |
| 2-4-860 | S | N—Ph | b1 | b1 | b1 | H |
| 2-4-861 | S | N—Ph | b2 | b2 | b1 | H |
| 2-4-862 | S | N—Ph | b3 | b3 | b1 | H |
| 2-4-863 | S | N—Ph | H | H | b2 | H |
| 2-4-864 | S | N—Ph | t-bu | t-bu | b2 | H |
| 2-4-865 | S | N—Ph | methyl | methyl | b2 | H |
| 2-4-866 | S | N—Ph | b1 | b1 | b2 | H |
| 2-4-867 | S | N—Ph | b2 | b2 | b2 | H |
| 2-4-868 | S | N—Ph | b3 | b3 | b2 | H |
| 2-4-869 | S | N—Ph | H | H | b3 | H |
| 2-4-870 | S | N—Ph | t-bu | t-bu | b3 | H |
| 2-4-871 | S | N—Ph | methyl | methyl | b3 | H |
| 2-4-872 | S | N—Ph | b1 | b1 | b3 | H |
| 2-4-873 | S | N—Ph | b2 | b2 | b3 | H |
| 2-4-874 | S | N—Ph | b3 | b3 | b3 | H |
| 2-4-875 | S | N—Ph | H | H | b4 | H |
| 2-4-876 | S | N—Ph | t-bu | t-bu | b4 | H |
| 2-4-877 | S | N—Ph | methyl | methyl | b4 | H |
| 2-4-878 | S | N—Ph | b1 | b1 | b4 | H |
| 2-4-879 | S | N—Ph | b2 | b2 | b4 | H |
| 2-4-880 | S | N—Ph | b3 | b3 | b4 | H |
| 2-4-881 | S | N—Ph | H | H | b5 | H |
| 2-4-882 | S | N—Ph | t-bu | t-bu | b5 | H |
| 2-4-883 | S | N—Ph | methyl | methyl | b5 | H |
| 2-4-884 | S | N—Ph | b1 | b1 | b5 | H |
| 2-4-885 | S | N—Ph | b2 | b2 | b5 | H |
| 2-4-886 | S | N—Ph | b3 | b3 | b5 | H |
| 2-4-887 | S | N—Ph | H | H | b6 | H |
| 2-4-888 | S | N—Ph | t-bu | t-bu | b6 | H |
| 2-4-889 | S | N—Ph | methyl | methyl | b6 | H |
| 2-4-890 | S | N—Ph | b1 | b1 | b6 | H |
| 2-4-891 | S | N—Ph | b2 | b2 | b6 | H |
| 2-4-892 | S | N—Ph | b3 | b3 | b6 | H |
| 2-4-893 | S | N—Ph | H | H | b7 | H |
| 2-4-894 | S | N—Ph | t-bu | t-bu | b7 | H |
| 2-4-895 | S | N—Ph | methyl | methyl | b7 | H |
| 2-4-896 | S | N—Ph | b1 | b1 | b7 | H |
| 2-4-897 | S | N—Ph | b2 | b2 | b7 | H |
| 2-4-898 | S | N—Ph | b3 | b3 | b7 | H |
| 2-4-899 | S | N—Ph | H | H | b8 | H |
| 2-4-900 | S | N—Ph | t-bu | t-bu | b8 | H |
| 2-4-901 | S | N—Ph | methyl | methyl | b8 | H |
| 2-4-902 | S | N—Ph | b1 | b1 | b8 | H |
| 2-4-903 | S | N—Ph | b2 | b2 | b8 | H |
| 2-4-904 | S | N—Ph | b3 | b3 | b8 | H |
| 2-4-905 | S | N—Ph | H | H | b9 | H |
| 2-4-906 | S | N—Ph | t-bu | t-bu | b9 | H |
| 2-4-907 | S | N—Ph | methyl | methyl | b9 | H |
| 2-4-908 | S | N—Ph | b1 | b1 | b9 | H |
| 2-4-909 | S | N—Ph | b2 | b2 | b9 | H |
| 2-4-910 | S | N—Ph | b3 | b3 | b9 | H |
| 2-4-911 | S | N—Ph | H | H | b10 | H |
| 2-4-912 | S | N—Ph | t-bu | t-bu | b10 | H |
| 2-4-913 | S | N—Ph | methyl | methyl | b10 | H |
| 2-4-914 | S | N—Ph | b1 | b1 | b10 | H |
| 2-4-915 | S | N—Ph | b2 | b2 | b10 | H |
| 2-4-916 | S | N—Ph | b3 | b3 | b10 | H |
| 2-4-917 | S | N—Ph | H | H | a1 | a1 |
| 2-4-918 | S | N—Ph | t-bu | t-bu | a1 | a1 |
| 2-4-919 | S | N—Ph | methyl | methyl | a1 | a1 |
| 2-4-920 | S | N—Ph | b1 | b1 | a1 | a1 |
| 2-4-921 | S | N—Ph | b2 | b2 | a1 | a1 |
| 2-4-922 | S | N—Ph | b3 | b3 | a1 | a1 |
| 2-4-923 | S | N—Ph | H | H | a2 | a2 |
| 2-4-924 | S | N—Ph | t-bu | t-bu | a2 | a2 |
| 2-4-925 | S | N—Ph | methyl | methyl | a2 | a2 |
| 2-4-926 | S | N—Ph | b1 | b1 | a2 | a2 |
| 2-4-927 | S | N—Ph | b2 | b2 | a2 | a2 |
| 2-4-928 | S | N—Ph | b3 | b3 | a2 | a2 |
| 2-4-929 | S | N—Ph | H | H | a3 | a3 |
| 2-4-930 | S | N—Ph | t-bu | t-bu | a3 | a3 |
| 2-4-931 | S | N—Ph | methyl | methyl | a3 | a3 |
| 2-4-932 | S | N—Ph | b1 | b1 | a3 | a3 |
| 2-4-933 | S | N—Ph | b2 | b2 | a3 | a3 |
| 2-4-934 | S | N—Ph | b3 | b3 | a3 | a3 |
| 2-4-935 | S | N—Ph | H | H | b1 | b1 |
| 2-4-936 | S | N—Ph | t-bu | t-bu | b1 | b1 |
| 2-4-937 | S | N—Ph | methyl | methyl | b1 | b1 |
| 2-4-938 | S | N—Ph | b1 | b1 | b1 | b1 |
| 2-4-939 | S | N—Ph | b2 | b2 | b1 | b1 |
| 2-4-940 | S | N—Ph | b3 | b3 | b1 | b1 |
| 2-4-941 | S | N—Ph | H | H | b2 | b2 |
| 2-4-942 | S | N—Ph | t-bu | t-bu | b2 | b2 |
| 2-4-943 | S | N—Ph | methyl | methyl | b2 | b2 |
| 2-4-944 | S | N—Ph | b1 | b1 | b2 | b2 |
| 2-4-945 | S | N—Ph | b2 | b2 | b2 | b2 |
| 2-4-946 | S | N—Ph | b3 | b3 | b2 | b2 |
| 2-4-947 | S | N—Ph | H | H | b3 | b3 |
| 2-4-948 | S | N—Ph | t-bu | t-bu | b3 | b3 |
| 2-4-949 | S | N—Ph | methyl | methyl | b3 | b3 |
| 2-4-950 | S | N—Ph | b1 | b1 | b3 | b3 |
| 2-4-951 | S | N—Ph | b2 | b2 | b3 | b3 |
| 2-4-952 | S | N—Ph | b3 | b3 | b3 | b3 |
| 2-4-953 | S | N—Ph | H | H | b5 | b5 |
| 2-4-954 | S | N—Ph | t-bu | t-bu | b5 | b5 |
| 2-4-955 | S | N—Ph | methyl | methyl | b5 | b5 |
| 2-4-956 | S | N—Ph | b1 | b1 | b5 | b5 |
| 2-4-957 | S | N—Ph | b2 | b2 | b5 | b5 |
| 2-4-958 | S | N—Ph | b3 | b3 | b5 | b5 |
| 2-4-959 | S | N—Ph | H | H | b6 | b6 |
| 2-4-960 | S | N—Ph | t-bu | t-bu | b6 | b6 |
| 2-4-961 | S | N—Ph | methyl | methyl | b6 | b6 |
| 2-4-962 | S | N—Ph | b1 | b1 | b6 | b6 |
| 2-4-963 | S | N—Ph | b2 | b2 | b6 | b6 |
| 2-4-964 | S | N—Ph | b3 | b3 | b6 | b6 |
| 2-4-965 | S | N—Ph | H | H | b7 | b7 |
| 2-4-966 | S | N—Ph | t-bu | t-bu | b7 | b7 |
| 2-4-967 | S | N—Ph | methyl | methyl | b7 | b7 |
| 2-4-968 | S | N—Ph | b1 | b1 | b7 | b7 |
| 2-4-969 | S | N—Ph | b2 | b2 | b7 | b7 |
| 2-4-970 | S | N—Ph | b3 | b3 | b7 | b7 |

Group 2-5
The compounds are represented by the formula 2-5 below, wherein CY₁ and CY₂ are each a benzene group, X₁ is B, and Y1, Y2, R3a, (R1)a1, (R2)a2 are each shown in the table 5 below, 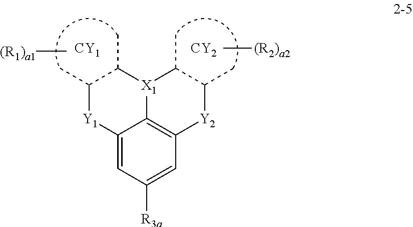
TABLE 5

| Compound | | | | | |
|---|---|---|---|---|---|
| No. | Y1 | Y2 | R3a | (R1)a1 | (R2)a2 |
| 2-5-001 | N-Ph | N-Ph | H | H | H |
| 2-5-002 | N-Ph | N-Ph | t-bu | H | H |
| 2-5-003 | N-Ph | N-Ph | methyl | H | H |
| 2-5-004 | N-Ph | N-Ph | t-bu | t-bu | t-bu |
| 2-5-005 | N-Ph | N-Ph | methyl | methyl | methyl |
| 2-5-006 | N-Ph | N-Ph | b1 | H | H |
| 2-5-007 | N-Ph | N-Ph | b2 | H | H |
| 2-5-008 | N-Ph | N-Ph | b3 | H | H |
| 2-5-009 | N-Ph | N-Ph | H | H | a1 |
| 2-5-010 | N-Ph | N-Ph | t-bu | H | a1 |
| 2-5-011 | N-Ph | N-Ph | methyl | H | a1 |
| 2-5-012 | N-Ph | N-Ph | b1 | H | a1 |
| 2-5-013 | N-Ph | N-Ph | b2 | H | a1 |
| 2-5-014 | N-Ph | N-Ph | b3 | H | a1 |
| 2-5-015 | N-Ph | N-Ph | H | H | a2 |
| 2-5-016 | N-Ph | N-Ph | t-bu | H | a2 |
| 2-5-017 | N-Ph | N-Ph | methyl | H | a2 |
| 2-5-018 | N-Ph | N-Ph | b1 | H | a2 |
| 2-5-019 | N-Ph | N-Ph | b2 | H | a2 |
| 2-5-020 | N-Ph | N-Ph | b3 | H | a2 |
| 2-5-021 | N-Ph | N-Ph | H | H | a3 |
| 2-5-022 | N-Ph | N-Ph | t-bu | H | a3 |
| 2-5-023 | N-Ph | N-Ph | methyl | H | a3 |
| 2-5-024 | N-Ph | N-Ph | b1 | H | a3 |
| 2-5-025 | N-Ph | N-Ph | b2 | H | a3 |
| 2-5-026 | N-Ph | N-Ph | b3 | H | a3 |
| 2-5-027 | N-Ph | N-Ph | H | H | a4 |
| 2-5-028 | N-Ph | N-Ph | t-bu | H | a4 |
| 2-5-029 | N-Ph | N-Ph | methyl | H | a4 |
| 2-5-030 | N-Ph | N-Ph | b1 | H | a4 |
| 2-5-031 | N-Ph | N-Ph | b2 | H | a4 |
| 2-5-032 | N-Ph | N-Ph | b3 | H | a4 |
| 2-5-033 | N-Ph | N-Ph | H | H | a5 |
| 2-5-034 | N-Ph | N-Ph | t-bu | H | a5 |
| 2-5-035 | N-Ph | N-Ph | methyl | H | a5 |
| 2-5-036 | N-Ph | N-Ph | b1 | H | a5 |
| 2-5-037 | N-Ph | N-Ph | b2 | H | a5 |
| 2-5-038 | N-Ph | N-Ph | b3 | H | a5 |
| 2-5-039 | N-Ph | N-Ph | H | H | a6 |
| 2-5-040 | N-Ph | N-Ph | t-bu | H | a6 |
| 2-5-041 | N-Ph | N-Ph | methyl | H | a6 |
| 2-5-042 | N-Ph | N-Ph | b1 | H | a6 |
| 2-5-043 | N-Ph | N-Ph | b2 | H | a6 |
| 2-5-044 | N-Ph | N-Ph | b3 | H | a6 |
| 2-5-045 | N-Ph | N-Ph | H | H | b1 |
| 2-5-046 | N-Ph | N-Ph | t-bu | H | b1 |
| 2-5-047 | N-Ph | N-Ph | methyl | H | b1 |
| 2-5-048 | N-Ph | N-Ph | b1 | H | b1 |
| 2-5-049 | N-Ph | N-Ph | b2 | H | b1 |
| 2-5-050 | N-Ph | N-Ph | b3 | H | b1 |
| 2-5-051 | N-Ph | N-Ph | H | H | b2 |
| 2-5-052 | N-Ph | N-Ph | t-bu | H | b2 |
| 2-5-053 | N-Ph | N-Ph | methyl | H | b2 |
| 2-5-054 | N-Ph | N-Ph | b1 | H | b2 |
| 2-5-055 | N-Ph | N-Ph | b2 | H | b2 |
| 2-5-056 | N-Ph | N-Ph | b3 | H | b2 |
| 2-5-057 | N-Ph | N-Ph | H | H | b3 |
| 2-5-058 | N-Ph | N-Ph | t-bu | H | b3 |
| 2-5-059 | N-Ph | N-Ph | methyl | H | b3 |
| 2-5-060 | N-Ph | N-Ph | b1 | H | b3 |
| 2-5-061 | N-Ph | N-Ph | b2 | H | b3 |
| 2-5-062 | N-Ph | N-Ph | b3 | H | b3 |
| 2-5-063 | N-Ph | N-Ph | H | H | b4 |
| 2-5-064 | N-Ph | N-Ph | t-bu | H | b4 |
| 2-5-065 | N-Ph | N-Ph | methyl | H | b4 |
| 2-5-066 | N-Ph | N-Ph | b1 | H | b4 |
| 2-5-067 | N-Ph | N-Ph | b2 | H | b4 |
| 2-5-068 | N-Ph | N-Ph | b3 | H | b4 |
| 2-5-069 | N-Ph | N-Ph | H | H | b5 |
| 2-5-070 | N-Ph | N-Ph | t-bu | H | b5 |
| 2-5-071 | N-Ph | N-Ph | methyl | H | b5 |
| 2-5-072 | N-Ph | N-Ph | b1 | H | b5 |
| 2-5-073 | N-Ph | N-Ph | b2 | H | b5 |
| 2-5-074 | N-Ph | N-Ph | b3 | H | b5 |
| 2-5-075 | N-Ph | N-Ph | H | H | b6 |
| 2-5-076 | N-Ph | N-Ph | t-bu | H | b6 |
| 2-5-077 | N-Ph | N-Ph | methyl | H | b6 |
| 2-5-078 | N-Ph | N-Ph | b1 | H | b6 |
| 2-5-079 | N-Ph | N-Ph | b2 | H | b6 |
| 2-5-080 | N-Ph | N-Ph | b3 | H | b6 |
| 2-5-081 | N-Ph | N-Ph | H | H | b7 |
| 2-5-082 | N-Ph | N-Ph | t-bu | H | b7 |
| 2-5-083 | N-Ph | N-Ph | methyl | H | b7 |
| 2-5-084 | N-Ph | N-Ph | b1 | H | b7 |
| 2-5-085 | N-Ph | N-Ph | b2 | H | b7 |
| 2-5-086 | N-Ph | N-Ph | b3 | H | b7 |
| 2-5-087 | N-Ph | N-Ph | H | H | b8 |
| 2-5-088 | N-Ph | N-Ph | t-bu | H | b8 |
| 2-5-089 | N-Ph | N-Ph | methyl | H | b8 |
| 2-5-090 | N-Ph | N-Ph | b1 | H | b8 |
| 2-5-091 | N-Ph | N-Ph | b2 | H | b8 |
| 2-5-092 | N-Ph | N-Ph | b3 | H | b8 |
| 2-5-093 | N-Ph | N-Ph | H | H | b9 |
| 2-5-094 | N-Ph | N-Ph | t-bu | H | b9 |
| 2-5-095 | N-Ph | N-Ph | methyl | H | b9 |
| 2-5-096 | N-Ph | N-Ph | b1 | H | b9 |
| 2-5-097 | N-Ph | N-Ph | b2 | H | b9 |
| 2-5-098 | N-Ph | N-Ph | b3 | H | b9 |
| 2-5-099 | N-Ph | N-Ph | H | H | b10 |
| 2-5-100 | N-Ph | N-Ph | t-bu | H | b10 |
| 2-5-101 | N-Ph | N-Ph | methyl | H | b10 |
| 2-5-102 | N-Ph | N-Ph | b1 | H | b10 |
| 2-5-103 | N-Ph | N-Ph | b2 | H | b10 |
| 2-5-104 | N-Ph | N-Ph | b3 | H | b10 |
| 2-5-105 | N-Ph | N-Ph | H | a1 | H |
| 2-5-106 | N-Ph | N-Ph | t-bu | a1 | H |
| 2-5-107 | N-Ph | N-Ph | methyl | a1 | H |
| 2-5-108 | N-Ph | N-Ph | b1 | a1 | H |
| 2-5-109 | N-Ph | N-Ph | b2 | a1 | H |
| 2-5-110 | N-Ph | N-Ph | b3 | a1 | H |
| 2-5-111 | N-Ph | N-Ph | H | a2 | H |
| 2-5-112 | N-Ph | N-Ph | t-bu | a2 | H |
| 2-5-113 | N-Ph | N-Ph | methyl | a2 | H |
| 2-5-114 | N-Ph | N-Ph | b1 | a2 | H |
| 2-5-115 | N-Ph | N-Ph | b2 | a2 | H |
| 2-5-116 | N-Ph | N-Ph | b3 | a2 | H |
| 2-5-117 | N-Ph | N-Ph | H | a3 | H |
| 2-5-118 | N-Ph | N-Ph | t-bu | a3 | H |
| 2-5-119 | N-Ph | N-Ph | methyl | a3 | H |
| 2-5-120 | N-Ph | N-Ph | b1 | a3 | H |
| 2-5-121 | N-Ph | N-Ph | b2 | a3 | H |
| 2-5-122 | N-Ph | N-Ph | b3 | a3 | H |
| 2-5-123 | N-Ph | N-Ph | H | a4 | H |
| 2-5-124 | N-Ph | N-Ph | t-bu | a4 | H |
| 2-5-125 | N-Ph | N-Ph | methyl | a4 | H |
| 2-5-126 | N-Ph | N-Ph | b1 | a4 | H |
| 2-5-127 | N-Ph | N-Ph | b2 | a4 | H |
| 2-5-128 | N-Ph | N-Ph | b3 | a4 | H |
| 2-5-129 | N-Ph | N-Ph | H | a5 | H |
| 2-5-130 | N-Ph | N-Ph | t-bu | a5 | H |
| 2-5-131 | N-Ph | N-Ph | methyl | a5 | H |
| 2-5-132 | N-Ph | N-Ph | b1 | a5 | H |
| 2-5-133 | N-Ph | N-Ph | b2 | a5 | H |
| 2-5-134 | N-Ph | N-Ph | b3 | a5 | H |
| 2-5-135 | N-Ph | N-Ph | H | a6 | H |
| 2-5-136 | N-Ph | N-Ph | t-bu | a6 | H |
| 2-5-137 | N-Ph | N-Ph | methyl | a6 | H |
| 2-5-138 | N-Ph | N-Ph | b1 | a6 | H |
| 2-5-139 | N-Ph | N-Ph | b2 | a6 | H |
| 2-5-140 | N-Ph | N-Ph | b3 | a6 | H |
| 2-5-141 | N-Ph | N-Ph | H | b1 | H |
| 2-5-142 | N-Ph | N-Ph | t-bu | b1 | H |
| 2-5-143 | N-Ph | N-Ph | methyl | b1 | H |
| 2-5-144 | N-Ph | N-Ph | b1 | b1 | H |
| 2-5-145 | N-Ph | N-Ph | b2 | b1 | H |
| 2-5-146 | N-Ph | N-Ph | b3 | b1 | H |
| 2-5-147 | N-Ph | N-Ph | H | b2 | H |
| 2-5-148 | N-Ph | N-Ph | t-bu | b2 | H |
| 2-5-149 | N-Ph | N-Ph | methyl | b2 | H |
| 2-5-150 | N-Ph | N-Ph | b1 | b2 | H |
| 2-5-151 | N-Ph | N-Ph | b2 | b2 | H |
| 2-5-152 | N-Ph | N-Ph | b3 | b2 | H |
| 2-5-153 | N-Ph | N-Ph | H | b3 | H |
| 2-5-154 | N-Ph | N-Ph | t-bu | b3 | H |
| 2-5-155 | N-Ph | N-Ph | methyl | b3 | H |
| 2-5-156 | N-Ph | N-Ph | b1 | b3 | H |
| 2-5-157 | N-Ph | N-Ph | b2 | b3 | H |
| 2-5-158 | N-Ph | N-Ph | b3 | b3 | H |
| 2-5-159 | N-Ph | N-Ph | H | b4 | H |
| 2-5-160 | N-Ph | N-Ph | t-bu | b4 | H |
| 2-5-161 | N-Ph | N-Ph | methyl | b4 | H |
| 2-5-162 | N-Ph | N-Ph | b1 | b4 | H |
| 2-5-163 | N-Ph | N-Ph | b2 | b4 | H |
| 2-5-164 | N-Ph | N-Ph | b3 | b4 | H |
| 2-5-165 | N-Ph | N-Ph | H | b5 | H |
| 2-5-166 | N-Ph | N-Ph | t-bu | b5 | H |
| 2-5-167 | N-Ph | N-Ph | methyl | b5 | H |
| 2-5-168 | N-Ph | N-Ph | b1 | b5 | H |
| 2-5-169 | N-Ph | N-Ph | b2 | b5 | H |
| 2-5-170 | N-Ph | N-Ph | b3 | b5 | H |
| 2-5-171 | N-Ph | N-Ph | H | b6 | H |
| 2-5-172 | N-Ph | N-Ph | t-bu | b6 | H |
| 2-5-173 | N-Ph | N-Ph | methyl | b6 | H |
| 2-5-174 | N-Ph | N-Ph | b1 | b6 | H |
| 2-5-175 | N-Ph | N-Ph | b2 | b6 | H |
| 2-5-176 | N-Ph | N-Ph | b3 | b6 | H |
| 2-5-177 | N-Ph | N-Ph | H | b7 | H |
| 2-5-178 | N-Ph | N-Ph | t-bu | b7 | H |
| 2-5-179 | N-Ph | N-Ph | methyl | b7 | H |
| 2-5-180 | N-Ph | N-Ph | b1 | b7 | H |
| 2-5-181 | N-Ph | N-Ph | b2 | b7 | H |
| 2-5-182 | N-Ph | N-Ph | b3 | b7 | H |
| 2-5-183 | N-Ph | N-Ph | H | b8 | H |
| 2-5-184 | N-Ph | N-Ph | t-bu | b8 | H |
| 2-5-185 | N-Ph | N-Ph | methyl | b8 | H |
| 2-5-186 | N-Ph | N-Ph | b1 | b8 | H |
| 2-5-187 | N-Ph | N-Ph | b2 | b8 | H |
| 2-5-188 | N-Ph | N-Ph | b3 | b8 | H |
| 2-5-189 | N-Ph | N-Ph | H | b9 | H |
| 2-5-190 | N-Ph | N-Ph | t-bu | b9 | H |
| 2-5-191 | N-Ph | N-Ph | methyl | b9 | H |
| 2-5-192 | N-Ph | N-Ph | b1 | b9 | H |
| 2-5-193 | N-Ph | N-Ph | b2 | b9 | H |
| 2-5-194 | N-Ph | N-Ph | b3 | b9 | H |
| 2-5-195 | N-Ph | N-Ph | H | b10 | H |
| 2-5-196 | N-Ph | N-Ph | t-bu | b10 | H |
| 2-5-197 | N-Ph | N-Ph | methyl | b10 | H |
| 2-5-198 | N-Ph | N-Ph | b1 | b10 | H |
| 2-5-199 | N-Ph | N-Ph | b2 | b10 | H |
| 2-5-200 | N-Ph | N-Ph | b3 | b10 | H |
| 2-5-201 | N-Ph | N-Ph | H | a1 | a1 |
| 2-5-202 | N-Ph | N-Ph | t-bu | a1 | a1 |
| 2-5-203 | N-Ph | N-Ph | methyl | a1 | a1 |
| 2-5-204 | N-Ph | N-Ph | b1 | a1 | a1 |
| 2-5-205 | N-Ph | N-Ph | b2 | a1 | a1 |
| 2-5-206 | N-Ph | N-Ph | b3 | a1 | a1 |
| 2-5-207 | N-Ph | N-Ph | H | a2 | a2 |
| 2-5-208 | N-Ph | N-Ph | t-bu | a2 | a2 |
| 2-5-209 | N-Ph | N-Ph | methyl | a2 | a2 |
| 2-5-210 | N-Ph | N-Ph | b1 | a2 | a2 |
| 2-5-211 | N-Ph | N-Ph | b2 | a2 | a2 |
| 2-5-212 | N-Ph | N-Ph | b3 | a2 | a2 |
| 2-5-213 | N-Ph | N-Ph | H | a3 | a3 |
| 2-5-214 | N-Ph | N-Ph | t-bu | a3 | a3 |
| 2-5-215 | N-Ph | N-Ph | methyl | a3 | a3 |
| 2-5-216 | N-Ph | N-Ph | b1 | a3 | a3 |
| 2-5-217 | N-Ph | N-Ph | b2 | a3 | a3 |
| 2-5-218 | N-Ph | N-Ph | b3 | a3 | a3 |
| 2-5-219 | N-Ph | N-Ph | H | b1 | b1 |
| 2-5-220 | N-Ph | N-Ph | t-bu | b1 | b1 |
| 2-5-221 | N-Ph | N-Ph | methyl | b1 | b1 |
| 2-5-222 | N-Ph | N-Ph | b1 | b1 | b1 |
| 2-5-223 | N-Ph | N-Ph | b2 | b1 | b1 |
| 2-5-224 | N-Ph | N-Ph | b3 | b1 | b1 |
| 2-5-225 | N-Ph | N-Ph | H | b2 | b2 |
| 2-5-226 | N-Ph | N-Ph | t-bu | b2 | b2 |
| 2-5-227 | N-Ph | N-Ph | methyl | b2 | b2 |
| 2-5-228 | N-Ph | N-Ph | b1 | b2 | b2 |
| 2-5-229 | N-Ph | N-Ph | b2 | b2 | b2 |
| 2-5-230 | N-Ph | N-Ph | b3 | b2 | b2 |
| 2-5-231 | N-Ph | N-Ph | H | b3 | b3 |
| 2-5-232 | N-Ph | N-Ph | t-bu | b3 | b3 |
| 2-5-233 | N-Ph | N-Ph | methyl | b3 | b3 |
| 2-5-234 | N-Ph | N-Ph | b1 | b3 | b3 |
| 2-5-235 | N-Ph | N-Ph | b2 | b3 | b3 |
| 2-5-236 | N-Ph | N-Ph | b3 | b3 | b3 |
| 2-5-237 | N-Ph | N-Ph | H | b5 | b5 |
| 2-5-238 | N-Ph | N-Ph | t-bu | b5 | b5 |
| 2-5-239 | N-Ph | N-Ph | methyl | b5 | b5 |
| 2-5-240 | N-Ph | N-Ph | b1 | b5 | b5 |
| 2-5-241 | N-Ph | N-Ph | b2 | b5 | b5 |
| 2-5-242 | N-Ph | N-Ph | b3 | b5 | b5 |
| 2-5-243 | N-Ph | N-Ph | H | b6 | b6 |
| 2-5-244 | N-Ph | N-Ph | t-bu | b6 | b6 |
| 2-5-245 | N-Ph | N-Ph | methyl | b6 | b6 |
| 2-5-246 | N-Ph | N-Ph | b1 | b6 | b6 |
| 2-5-247 | N-Ph | N-Ph | b2 | b6 | b6 |
| 2-5-248 | N-Ph | N-Ph | b3 | b6 | b6 |
| 2-5-249 | N-Ph | N-Ph | H | b7 | b7 |
| 2-5-250 | N-Ph | N-Ph | t-bu | b7 | b7 |
| 2-5-251 | N-Ph | N-Ph | methyl | b7 | b7 |
| 2-5-252 | N-Ph | N-Ph | b1 | b7 | b7 |
| 2-5-253 | N-Ph | N-Ph | b2 | b7 | b7 |
| 2-5-254 | N-Ph | N-Ph | b3 | b7 | b7 |
| 2-5-255 | N-tolyl | N-tolyl | H | H | H |
| 2-5-256 | N-tolyl | N-tolyl | t-bu | H | H |
| 2-5-257 | N-tolyl | N-tolyl | methyl | H | H |
| 2-5-258 | N-tolyl | N-tolyl | t-bu | t-bu | t-bu |
| 2-5-259 | N-tolyl | N-tolyl | methyl | methyl | methyl |
| 2-5-260 | N-tolyl | N-tolyl | b1 | H | H |
| 2-5-261 | N-tolyl | N-tolyl | b2 | H | H |
| 2-5-262 | N-tolyl | N-tolyl | b3 | H | H |
| 2-5-263 | N-tolyl | N-tolyl | H | H | a1 |
| 2-5-264 | N-tolyl | N-tolyl | t-bu | H | a1 |
| 2-5-265 | N-tolyl | N-tolyl | methyl | H | a1 |
| 2-5-266 | N-tolyl | N-tolyl | b1 | H | a1 |
| 2-5-267 | N-tolyl | N-tolyl | b2 | H | a1 |
| 2-5-268 | N-tolyl | N-tolyl | b3 | H | a1 |
| 2-5-269 | N-tolyl | N-tolyl | H | H | a2 |
| 2-5-270 | N-tolyl | N-tolyl | t-bu | H | a2 |
| 2-5-271 | N-tolyl | N-tolyl | methyl | H | a2 |
| 2-5-272 | N-tolyl | N-tolyl | b1 | H | a2 |
| 2-5-273 | N-tolyl | N-tolyl | b2 | H | a2 |
| 2-5-274 | N-tolyl | N-tolyl | b3 | H | a2 |
| 2-5-275 | N-tolyl | N-tolyl | H | H | a3 |
| 2-5-276 | N-tolyl | N-tolyl | t-bu | H | a3 |
| 2-5-277 | N-tolyl | N-tolyl | methyl | H | a3 |
| 2-5-278 | N-tolyl | N-tolyl | b1 | H | a3 |
| 2-5-279 | N-tolyl | N-tolyl | b2 | H | a3 |
| 2-5-280 | N-tolyl | N-tolyl | b3 | H | a3 |
| 2-5-281 | N-tolyl | N-tolyl | H | H | a4 |
| 2-5-282 | N-tolyl | N-tolyl | t-bu | H | a4 |
| 2-5-283 | N-tolyl | N-tolyl | methyl | H | a4 |
| 2-5-284 | N-tolyl | N-tolyl | b1 | H | a4 |
| 2-5-285 | N-tolyl | N-tolyl | b2 | H | a4 |
| 2-5-286 | N-tolyl | N-tolyl | b3 | H | a4 |
| 2-5-287 | N-tolyl | N-tolyl | H | H | a5 |
| 2-5-288 | N-tolyl | N-tolyl | t-bu | H | a5 |
| 2-5-289 | N-tolyl | N-tolyl | methyl | H | a5 |
| 2-5-290 | N-tolyl | N-tolyl | b1 | H | a5 |
| 2-5-291 | N-tolyl | N-tolyl | b2 | H | a5 |
| 2-5-292 | N-tolyl | N-tolyl | b3 | H | a5 |
| 2-5-293 | N-tolyl | N-tolyl | H | H | a6 |
| 2-5-294 | N-tolyl | N-tolyl | t-bu | H | a6 |
| 2-5-295 | N-tolyl | N-tolyl | methyl | H | a6 |
| 2-5-296 | N-tolyl | N-tolyl | b1 | H | a6 |
| 2-5-297 | N-tolyl | N-tolyl | b2 | H | a6 |
| 2-5-298 | N-tolyl | N-tolyl | b3 | H | a6 |
| 2-5-299 | N-tolyl | N-tolyl | H | H | b1 |
| 2-5-300 | N-tolyl | N-tolyl | t-bu | H | b1 |
| 2-5-301 | N-tolyl | N-tolyl | methyl | H | b1 |
| 2-5-302 | N-tolyl | N-tolyl | b1 | H | b1 |
| 2-5-303 | N-tolyl | N-tolyl | b2 | H | b1 |
| 2-5-304 | N-tolyl | N-tolyl | b3 | H | b1 |
| 2-5-305 | N-tolyl | N-tolyl | H | H | b2 |
| 2-5-306 | N-tolyl | N-tolyl | t-bu | H | b2 |
| 2-5-307 | N-tolyl | N-tolyl | methyl | H | b2 |
| 2-5-308 | N-tolyl | N-tolyl | b1 | H | b2 |
| 2-5-309 | N-tolyl | N-tolyl | b2 | H | b2 |
| 2-5-310 | N-tolyl | N-tolyl | b3 | H | b2 |
| 2-5-311 | N-tolyl | N-tolyl | H | H | b3 |
| 2-5-312 | N-tolyl | N-tolyl | t-bu | H | b3 |
| 2-5-313 | N-tolyl | N-tolyl | methyl | H | b3 |
| 2-5-314 | N-tolyl | N-tolyl | b1 | H | b3 |
| 2-5-315 | N-tolyl | N-tolyl | b2 | H | b3 |
| 2-5-316 | N-tolyl | N-tolyl | b3 | H | b3 |
| 2-5-317 | N-tolyl | N-tolyl | H | H | b4 |
| 2-5-318 | N-tolyl | N-tolyl | t-bu | H | b4 |
| 2-5-319 | N-tolyl | N-tolyl | methyl | H | b4 |
| 2-5-320 | N-tolyl | N-tolyl | b1 | H | b4 |
| 2-5-321 | N-tolyl | N-tolyl | b2 | H | b4 |
| 2-5-322 | N-tolyl | N-tolyl | b3 | H | b4 |
| 2-5-323 | N-tolyl | N-tolyl | H | H | b5 |
| 2-5-324 | N-tolyl | N-tolyl | t-bu | H | b5 |
| 2-5-325 | N-tolyl | N-tolyl | methyl | H | b5 |
| 2-5-326 | N-tolyl | N-tolyl | b1 | H | b5 |
| 2-5-327 | N-tolyl | N-tolyl | b2 | H | b5 |
| 2-5-328 | N-tolyl | N-tolyl | b3 | H | b5 |
| 2-5-329 | N-tolyl | N-tolyl | H | H | b6 |
| 2-5-330 | N-tolyl | N-tolyl | t-bu | H | b6 |
| 2-5-331 | N-tolyl | N-tolyl | methyl | H | b6 |
| 2-5-332 | N-tolyl | N-tolyl | b1 | H | b6 |
| 2-5-333 | N-tolyl | N-tolyl | b2 | H | b6 |
| 2-5-334 | N-tolyl | N-tolyl | b3 | H | b6 |
| 2-5-335 | N-tolyl | N-tolyl | H | H | b7 |
| 2-5-336 | N-tolyl | N-tolyl | t-bu | H | b7 |
| 2-5-337 | N-tolyl | N-tolyl | methyl | H | b7 |
| 2-5-338 | N-tolyl | N-tolyl | b1 | H | b7 |
| 2-5-339 | N-tolyl | N-tolyl | b2 | H | b7 |
| 2-5-340 | N-tolyl | N-tolyl | b3 | H | b7 |
| 2-5-341 | N-tolyl | N-tolyl | H | H | b8 |
| 2-5-342 | N-tolyl | N-tolyl | t-bu | H | b8 |
| 2-5-343 | N-tolyl | N-tolyl | methyl | H | b8 |
| 2-5-344 | N-tolyl | N-tolyl | b1 | H | b8 |
| 2-5-345 | N-tolyl | N-tolyl | b2 | H | b8 |
| 2-5-346 | N-tolyl | N-tolyl | b3 | H | b8 |
| 2-5-347 | N-tolyl | N-tolyl | H | H | b9 |
| 2-5-348 | N-tolyl | N-tolyl | t-bu | H | b9 |
| 2-5-349 | N-tolyl | N-tolyl | methyl | H | b9 |
| 2-5-350 | N-tolyl | N-tolyl | b1 | H | b9 |
| 2-5-351 | N-tolyl | N-tolyl | b2 | H | b9 |
| 2-5-352 | N-tolyl | N-tolyl | b3 | H | b9 |
| 2-5-353 | N-tolyl | N-tolyl | H | H | b10 |
| 2-5-354 | N-tolyl | N-tolyl | t-bu | H | b10 |
| 2-5-355 | N-tolyl | N-tolyl | methyl | H | b10 |
| 2-5-356 | N-tolyl | N-tolyl | b1 | H | b10 |
| 2-5-357 | N-tolyl | N-tolyl | b2 | H | b10 |
| 2-5-358 | N-tolyl | N-tolyl | b3 | H | b10 |
| 2-5-359 | N-tolyl | N-tolyl | H | a1 | H |
| 2-5-360 | N-tolyl | N-tolyl | t-bu | a1 | H |
| 2-5-361 | N-tolyl | N-tolyl | methyl | a1 | H |
| 2-5-362 | N-tolyl | N-tolyl | b1 | a1 | H |
| 2-5-363 | N-tolyl | N-tolyl | b2 | a1 | H |
| 2-5-364 | N-tolyl | N-tolyl | b3 | a1 | H |
| 2-5-365 | N-tolyl | N-tolyl | H | a2 | H |
| 2-5-366 | N-tolyl | N-tolyl | t-bu | a2 | H |
| 2-5-367 | N-tolyl | N-tolyl | methyl | a2 | H |
| 2-5-368 | N-tolyl | N-tolyl | b1 | a2 | H |
| 2-5-369 | N-tolyl | N-tolyl | b2 | a2 | H |
| 2-5-370 | N-tolyl | N-tolyl | b3 | a2 | H |
| 2-5-371 | N-tolyl | N-tolyl | H | a3 | H |
| 2-5-372 | N-tolyl | N-tolyl | t-bu | a3 | H |
| 2-5-373 | N-tolyl | N-tolyl | methyl | a3 | H |
| 2-5-374 | N-tolyl | N-tolyl | b1 | a3 | H |
| 2-5-375 | N-tolyl | N-tolyl | b2 | a3 | H |
| 2-5-376 | N-tolyl | N-tolyl | b3 | a3 | H |
| 2-5-377 | N-tolyl | N-tolyl | H | a4 | H |
| 2-5-378 | N-tolyl | N-tolyl | t-bu | a4 | H |
| 2-5-379 | N-tolyl | N-tolyl | methyl | a4 | H |
| 2-5-380 | N-tolyl | N-tolyl | b1 | a4 | H |
| 2-5-381 | N-tolyl | N-tolyl | b2 | a4 | H |
| 2-5-382 | N-tolyl | N-tolyl | b3 | a4 | H |
| 2-5-383 | N-tolyl | N-tolyl | H | a5 | H |
| 2-5-384 | N-tolyl | N-tolyl | t-bu | a5 | H |
| 2-5-385 | N-tolyl | N-tolyl | methyl | a5 | H |
| 2-5-386 | N-tolyl | N-tolyl | b1 | a5 | H |
| 2-5-387 | N-tolyl | N-tolyl | b2 | a5 | H |
| 2-5-388 | N-tolyl | N-tolyl | b3 | a5 | H |
| 2-5-389 | N-tolyl | N-tolyl | H | a6 | H |
| 2-5-390 | N-tolyl | N-tolyl | t-bu | a6 | H |
| 2-5-391 | N-tolyl | N-tolyl | methyl | a6 | H |
| 2-5-392 | N-tolyl | N-tolyl | b1 | a6 | H |
| 2-5-393 | N-tolyl | N-tolyl | b2 | a6 | H |
| 2-5-394 | N-tolyl | N-tolyl | b3 | a6 | H |
| 2-5-395 | N-tolyl | N-tolyl | H | b1 | H |
| 2-5-396 | N-tolyl | N-tolyl | t-bu | b1 | H |
| 2-5-397 | N-tolyl | N-tolyl | methyl | b1 | H |
| 2-5-398 | N-tolyl | N-tolyl | b1 | b1 | H |
| 2-5-399 | N-tolyl | N-tolyl | b2 | b1 | H |
| 2-5-400 | N-tolyl | N-tolyl | b3 | b1 | H |
| 2-5-401 | N-tolyl | N-tolyl | H | b2 | H |
| 2-5-402 | N-tolyl | N-tolyl | t-bu | b2 | H |
| 2-5-403 | N-tolyl | N-tolyl | methyl | b2 | H |
| 2-5-404 | N-tolyl | N-tolyl | b1 | b2 | H |
| 2-5-405 | N-tolyl | N-tolyl | b2 | b2 | H |
| 2-5-406 | N-tolyl | N-tolyl | b3 | b2 | H |
| 2-5-407 | N-tolyl | N-tolyl | H | b3 | H |
| 2-5-408 | N-tolyl | N-tolyl | t-bu | b3 | H |
| 2-5-409 | N-tolyl | N-tolyl | methyl | b3 | H |
| 2-5-410 | N-tolyl | N-tolyl | b1 | b3 | H |
| 2-5-411 | N-tolyl | N-tolyl | b2 | b3 | H |
| 2-5-412 | N-tolyl | N-tolyl | b3 | b3 | H |
| 2-5-413 | N-tolyl | N-tolyl | H | b4 | H |
| 2-5-414 | N-tolyl | N-tolyl | t-bu | b4 | H |
| 2-5-415 | N-tolyl | N-tolyl | methyl | b4 | H |
| 2-5-416 | N-tolyl | N-tolyl | b1 | b4 | H |
| 2-5-417 | N-tolyl | N-tolyl | b2 | b4 | H |
| 2-5-418 | N-tolyl | N-tolyl | b3 | b4 | H |
| 2-5-419 | N-tolyl | N-tolyl | H | b5 | H |
| 2-5-420 | N-tolyl | N-tolyl | t-bu | b5 | H |
| 2-5-421 | N-tolyl | N-tolyl | methyl | b5 | H |
| 2-5-422 | N-tolyl | N-tolyl | b1 | b5 | H |
| 2-5-423 | N-tolyl | N-tolyl | b2 | b5 | H |
| 2-5-424 | N-tolyl | N-tolyl | b3 | b5 | H |
| 2-5-425 | N-tolyl | N-tolyl | H | b6 | H |
| 2-5-426 | N-tolyl | N-tolyl | t-bu | b6 | H |
| 2-5-427 | N-tolyl | N-tolyl | methyl | b6 | H |
| 2-5-428 | N-tolyl | N-tolyl | b1 | b6 | H |
| 2-5-429 | N-tolyl | N-tolyl | b2 | b6 | H |
| 2-5-430 | N-tolyl | N-tolyl | b3 | b6 | H |
| 2-5-431 | N-tolyl | N-tolyl | H | b7 | H |
| 2-5-432 | N-tolyl | N-tolyl | t-bu | b7 | H |
| 2-5-433 | N-tolyl | N-tolyl | methyl | b7 | H |
| 2-5-434 | N-tolyl | N-tolyl | b1 | b7 | H |
| 2-5-435 | N-tolyl | N-tolyl | b2 | b7 | H |
| 2-5-436 | N-tolyl | N-tolyl | b3 | b7 | H |
| 2-5-437 | N-tolyl | N-tolyl | H | b8 | H |
| 2-5-438 | N-tolyl | N-tolyl | t-bu | b8 | H |
| 2-5-439 | N-tolyl | N-tolyl | methyl | b8 | H |
| 2-5-440 | N-tolyl | N-tolyl | b1 | b8 | H |
| 2-5-441 | N-tolyl | N-tolyl | b2 | b8 | H |
| 2-5-442 | N-tolyl | N-tolyl | b3 | b8 | H |
| 2-5-443 | N-tolyl | N-tolyl | H | b9 | H |
| 2-5-444 | N-tolyl | N-tolyl | t-bu | b9 | H |
| 2-5-445 | N-tolyl | N-tolyl | methyl | b9 | H |
| 2-5-446 | N-tolyl | N-tolyl | b1 | b9 | H |
| 2-5-447 | N-tolyl | N-tolyl | b2 | b9 | H |
| 2-5-448 | N-tolyl | N-tolyl | b3 | b9 | H |
| 2-5-449 | N-tolyl | N-tolyl | H | b10 | H |
| 2-5-450 | N-tolyl | N-tolyl | t-bu | b10 | H |
| 2-5-451 | N-tolyl | N-tolyl | methyl | b10 | H |
| 2-5-452 | N-tolyl | N-tolyl | b1 | b10 | H |
| 2-5-453 | N-tolyl | N-tolyl | b2 | b10 | H |
| 2-5-454 | N-tolyl | N-tolyl | b3 | b10 | H |
| 2-5-455 | N-tolyl | N-tolyl | H | a1 | a1 |
| 2-5-456 | N-tolyl | N-tolyl | t-bu | a1 | a1 |
| 2-5-457 | N-tolyl | N-tolyl | methyl | a1 | a1 |
| 2-5-458 | N-tolyl | N-tolyl | b1 | a1 | a1 |
| 2-5-459 | N-tolyl | N-tolyl | b2 | a1 | a1 |
| 2-5-460 | N-tolyl | N-tolyl | b3 | a1 | a1 |
| 2-5-461 | N-tolyl | N-tolyl | H | a2 | a2 |
| 2-5-462 | N-tolyl | N-tolyl | t-bu | a2 | a2 |
| 2-5-463 | N-tolyl | N-tolyl | methyl | a2 | a2 |
| 2-5-464 | N-tolyl | N-tolyl | b1 | a2 | a2 |
| 2-5-465 | N-tolyl | N-tolyl | b2 | a2 | a2 |
| 2-5-466 | N-tolyl | N-tolyl | b3 | a2 | a2 |
| 2-5-467 | N-tolyl | N-tolyl | H | a3 | a3 |
| 2-5-468 | N-tolyl | N-tolyl | t-bu | a3 | a3 |
| 2-5-469 | N-tolyl | N-tolyl | methyl | a3 | a3 |
| 2-5-470 | N-tolyl | N-tolyl | b1 | a3 | a3 |
| 2-5-471 | N-tolyl | N-tolyl | b2 | a3 | a3 |
| 2-5-472 | N-tolyl | N-tolyl | b3 | a3 | a3 |
| 2-5-473 | N-tolyl | N-tolyl | H | b1 | b1 |
| 2-5-474 | N-tolyl | N-tolyl | t-bu | b1 | b1 |
| 2-5-475 | N-tolyl | N-tolyl | methyl | b1 | b1 |
| 2-5-476 | N-tolyl | N-tolyl | b1 | b1 | b1 |
| 2-5-477 | N-tolyl | N-tolyl | b2 | b1 | b1 |
| 2-5-478 | N-tolyl | N-tolyl | b3 | b1 | b1 |
| 2-5-479 | N-tolyl | N-tolyl | H | b2 | b2 |
| 2-5-480 | N-tolyl | N-tolyl | t-bu | b2 | b2 |
| 2-5-481 | N-tolyl | N-tolyl | methyl | b2 | b2 |
| 2-5-482 | N-tolyl | N-tolyl | b1 | b2 | b2 |
| 2-5-483 | N-tolyl | N-tolyl | b2 | b2 | b2 |
| 2-5-484 | N-tolyl | N-tolyl | b3 | b2 | b2 |
| 2-5-485 | N-tolyl | N-tolyl | H | b3 | b3 |
| 2-5-486 | N-tolyl | N-tolyl | t-bu | b3 | b3 |
| 2-5-487 | N-tolyl | N-tolyl | methyl | b3 | b3 |
| 2-5-488 | N-tolyl | N-tolyl | b1 | b3 | b3 |
| 2-5-489 | N-tolyl | N-tolyl | b2 | b3 | b3 |
| 2-5-490 | N-tolyl | N-tolyl | b3 | b3 | b3 |
| 2-5-491 | N-tolyl | N-tolyl | H | b5 | b5 |
| 2-5-492 | N-tolyl | N-tolyl | t-bu | b5 | b5 |
| 2-5-493 | N-tolyl | N-tolyl | methyl | b5 | b5 |
| 2-5-494 | N-tolyl | N-tolyl | b1 | b5 | b5 |
| 2-5-495 | N-tolyl | N-tolyl | b2 | b5 | b5 |
| 2-5-496 | N-tolyl | N-tolyl | b3 | b5 | b5 |
| 2-5-497 | N-tolyl | N-tolyl | H | b6 | b6 |
| 2-5-498 | N-tolyl | N-tolyl | t-bu | b6 | b6 |
| 2-5-499 | N-tolyl | N-tolyl | methyl | b6 | b6 |
| 2-5-500 | N-tolyl | N-tolyl | b1 | b6 | b6 |
| 2-5-501 | N-tolyl | N-tolyl | b2 | b6 | b6 |
| 2-5-502 | N-tolyl | N-tolyl | b3 | b6 | b6 |
| 2-5-503 | N-tolyl | N-tolyl | H | b7 | b7 |
| 2-5-504 | N-tolyl | N-tolyl | t-bu | b7 | b7 |
| 2-5-505 | N-tolyl | N-tolyl | methyl | b7 | b7 |
| 2-5-506 | N-tolyl | N-tolyl | b1 | b7 | b7 |
| 2-5-507 | N-tolyl | N-tolyl | b2 | b7 | b7 |
| 2-5-508 | N-tolyl | N-tolyl | b3 | b7 | b7 |
| 2-5-509 | N-Biph | N-Biph | H | H | H |
| 2-5-510 | N-Biph | N-Biph | t-bu | H | H |
| 2-5-511 | N-Biph | N-Biph | methyl | H | H |
| 2-5-512 | N-Biph | N-Biph | t-bu | t-bu | t-bu |
| 2-5-513 | N-Biph | N-Biph | methyl | methyl | methyl |
| 2-5-514 | N-Biph | N-Biph | b1 | H | H |
| 2-5-515 | N-Biph | N-Biph | b2 | H | H |
| 2-5-516 | N-Biph | N-Biph | b3 | H | H |
| 2-5-517 | N-Biph | N-Biph | H | H | a1 |
| 2-5-518 | N-Biph | N-Biph | t-bu | H | a1 |
| 2-5-519 | N-Biph | N-Biph | methyl | H | a1 |
| 2-5-520 | N-Biph | N-Biph | b1 | H | a1 |
| 2-5-521 | N-Biph | N-Biph | b2 | H | a1 |
| 2-5-522 | N-Biph | N-Biph | b3 | H | a1 |
| 2-5-523 | N-Biph | N-Biph | H | H | a2 |
| 2-5-524 | N-Biph | N-Biph | t-bu | H | a2 |
| 2-5-525 | N-Biph | N-Biph | methyl | H | a2 |
| 2-5-526 | N-Biph | N-Biph | b1 | H | a2 |
| 2-5-527 | N-Biph | N-Biph | b2 | H | a2 |
| 2-5-528 | N-Biph | N-Biph | b3 | H | a2 |
| 2-5-529 | N-Biph | N-Biph | H | H | a3 |
| 2-5-530 | N-Biph | N-Biph | t-bu | H | a3 |
| 2-5-531 | N-Biph | N-Biph | methyl | H | a3 |
| 2-5-532 | N-Biph | N-Biph | b1 | H | a3 |
| 2-5-533 | N-Biph | N-Biph | b2 | H | a3 |
| 2-5-534 | N-Biph | N-Biph | b3 | H | a3 |
| 2-5-535 | N-Biph | N-Biph | H | H | a4 |
| 2-5-536 | N-Biph | N-Biph | t-bu | H | a4 |
| 2-5-537 | N-Biph | N-Biph | methyl | H | a4 |
| 2-5-538 | N-Biph | N-Biph | b1 | H | a4 |
| 2-5-539 | N-Biph | N-Biph | b2 | H | a4 |
| 2-5-540 | N-Biph | N-Biph | b3 | H | a4 |
| 2-5-541 | N-Biph | N-Biph | H | H | a5 |
| 2-5-542 | N-Biph | N-Biph | t-bu | H | a5 |
| 2-5-543 | N-Biph | N-Biph | methyl | H | a5 |
| 2-5-544 | N-Biph | N-Biph | b1 | H | a5 |
| 2-5-545 | N-Biph | N-Biph | b2 | H | a5 |
| 2-5-546 | N-Biph | N-Biph | b3 | H | a5 |
| 2-5-547 | N-Biph | N-Biph | H | H | a6 |
| 2-5-548 | N-Biph | N-Biph | t-bu | H | a6 |
| 2-5-549 | N-Biph | N-Biph | methyl | H | a6 |
| 2-5-550 | N-Biph | N-Biph | b1 | H | a6 |
| 2-5-551 | N-Biph | N-Biph | b2 | H | a6 |
| 2-5-552 | N-Biph | N-Biph | b3 | H | a6 |
| 2-5-553 | N-Biph | N-Biph | H | H | b1 |
| 2-5-554 | N-Biph | N-Biph | t-bu | H | b1 |
| 2-5-555 | N-Biph | N-Biph | methyl | H | b1 |
| 2-5-556 | N-Biph | N-Biph | b1 | H | b1 |
| 2-5-557 | N-Biph | N-Biph | b2 | H | b1 |
| 2-5-558 | N-Biph | N-Biph | b3 | H | b1 |
| 2-5-559 | N-Biph | N-Biph | H | H | b2 |
| 2-5-560 | N-Biph | N-Biph | t-bu | H | b2 |
| 2-5-561 | N-Biph | N-Biph | methyl | H | b2 |
| 2-5-562 | N-Biph | N-Biph | b1 | H | b2 |
| 2-5-563 | N-Biph | N-Biph | b2 | H | b2 |
| 2-5-564 | N-Biph | N-Biph | b3 | H | b2 |
| 2-5-565 | N-Biph | N-Biph | H | H | b3 |
| 2-5-566 | N-Biph | N-Biph | t-bu | H | b3 |
| 2-5-567 | N-Biph | N-Biph | methyl | H | b3 |
| 2-5-568 | N-Biph | N-Biph | b1 | H | b3 |
| 2-5-569 | N-Biph | N-Biph | b2 | H | b3 |
| 2-5-570 | N-Biph | N-Biph | b3 | H | b3 |
| 2-5-571 | N-Biph | N-Biph | H | H | b4 |
| 2-5-572 | N-Biph | N-Biph | t-bu | H | b4 |
| 2-5-573 | N-Biph | N-Biph | methyl | H | b4 |
| 2-5-574 | N-Biph | N-Biph | b1 | H | b4 |
| 2-5-575 | N-Biph | N-Biph | b2 | H | b4 |
| 2-5-576 | N-Biph | N-Biph | b3 | H | b4 |
| 2-5-577 | N-Biph | N-Biph | H | H | b5 |
| 2-5-578 | N-Biph | N-Biph | t-bu | H | b5 |
| 2-5-579 | N-Biph | N-Biph | methyl | H | b5 |
| 2-5-580 | N-Biph | N-Biph | b1 | H | b5 |
| 2-5-581 | N-Biph | N-Biph | b2 | H | b5 |
| 2-5-582 | N-Biph | N-Biph | b3 | H | b5 |
| 2-5-583 | N-Biph | N-Biph | H | H | b6 |
| 2-5-584 | N-Biph | N-Biph | t-bu | H | b6 |
| 2-5-585 | N-Biph | N-Biph | methyl | H | b6 |
| 2-5-586 | N-Biph | N-Biph | b1 | H | b6 |
| 2-5-587 | N-Biph | N-Biph | b2 | H | b6 |
| 2-5-588 | N-Biph | N-Biph | b3 | H | b6 |
| 2-5-589 | N-Biph | N-Biph | H | H | b7 |
| 2-5-590 | N-Biph | N-Biph | t-bu | H | b7 |
| 2-5-591 | N-Biph | N-Biph | methyl | H | b7 |
| 2-5-592 | N-Biph | N-Biph | b1 | H | b7 |
| 2-5-593 | N-Biph | N-Biph | b2 | H | b7 |
| 2-5-594 | N-Biph | N-Biph | b3 | H | b7 |
| 2-5-595 | N-Biph | N-Biph | H | H | b8 |
| 2-5-596 | N-Biph | N-Biph | t-bu | H | b8 |
| 2-5-597 | N-Biph | N-Biph | methyl | H | b8 |
| 2-5-598 | N-Biph | N-Biph | b1 | H | b8 |
| 2-5-599 | N-Biph | N-Biph | b2 | H | b8 |
| 2-5-600 | N-Biph | N-Biph | b3 | H | b8 |
| 2-5-601 | N-Biph | N-Biph | H | H | b9 |
| 2-5-602 | N-Biph | N-Biph | t-bu | H | b9 |
| 2-5-603 | N-Biph | N-Biph | methyl | H | b9 |
| 2-5-604 | N-Biph | N-Biph | b1 | H | b9 |
| 2-5-605 | N-Biph | N-Biph | b2 | H | b9 |
| 2-5-606 | N-Biph | N-Biph | b3 | H | b9 |
| 2-5-607 | N-Biph | N-Biph | H | H | b10 |
| 2-5-608 | N-Biph | N-Biph | t-bu | H | b10 |
| 2-5-609 | N-Biph | N-Biph | methyl | H | b10 |
| 2-5-610 | N-Biph | N-Biph | b1 | H | b10 |
| 2-5-611 | N-Biph | N-Biph | b2 | H | b10 |
| 2-5-612 | N-Biph | N-Biph | b3 | H | b10 |
| 2-5-613 | N-Biph | N-Biph | H | a1 | H |
| 2-5-614 | N-Biph | N-Biph | t-bu | a1 | H |
| 2-5-615 | N-Biph | N-Biph | methyl | a1 | H |
| 2-5-616 | N-Biph | N-Biph | b1 | a1 | H |
| 2-5-617 | N-Biph | N-Biph | b2 | a1 | H |
| 2-5-618 | N-Biph | N-Biph | b3 | a1 | H |
| 2-5-619 | N-Biph | N-Biph | H | a2 | H |
| 2-5-620 | N-Biph | N-Biph | t-bu | a2 | H |
| 2-5-621 | N-Biph | N-Biph | methyl | a2 | H |
| 2-5-622 | N-Biph | N-Biph | b1 | a2 | H |
| 2-5-623 | N-Biph | N-Biph | b2 | a2 | H |
| 2-5-624 | N-Biph | N-Biph | b3 | a2 | H |
| 2-5-625 | N-Biph | N-Biph | H | a3 | H |
| 2-5-626 | N-Biph | N-Biph | t-bu | a3 | H |
| 2-5-627 | N-Biph | N-Biph | methyl | a3 | H |
| 2-5-628 | N-Biph | N-Biph | b1 | a3 | H |
| 2-5-629 | N-Biph | N-Biph | b2 | a3 | H |
| 2-5-630 | N-Biph | N-Biph | b3 | a3 | H |
| 2-5-631 | N-Biph | N-Biph | H | a4 | H |
| 2-5-632 | N-Biph | N-Biph | t-bu | a4 | H |
| 2-5-633 | N-Biph | N-Biph | methyl | a4 | H |
| 2-5-634 | N-Biph | N-Biph | b1 | a4 | H |
| 2-5-635 | N-Biph | N-Biph | b2 | a4 | H |
| 2-5-636 | N-Biph | N-Biph | b3 | a4 | H |
| 2-5-637 | N-Biph | N-Biph | H | a5 | H |
| 2-5-638 | N-Biph | N-Biph | t-bu | a5 | H |
| 2-5-639 | N-Biph | N-Biph | methyl | a5 | H |
| 2-5-640 | N-Biph | N-Biph | b1 | a5 | H |
| 2-5-641 | N-Biph | N-Biph | b2 | a5 | H |
| 2-5-642 | N-Biph | N-Biph | b3 | a5 | H |
| 2-5-643 | N-Biph | N-Biph | H | a6 | H |
| 2-5-644 | N-Biph | N-Biph | t-bu | a6 | H |
| 2-5-645 | N-Biph | N-Biph | methyl | a6 | H |
| 2-5-646 | N-Biph | N-Biph | b1 | a6 | H |
| 2-5-647 | N-Biph | N-Biph | b2 | a6 | H |
| 2-5-648 | N-Biph | N-Biph | b3 | a6 | H |
| 2-5-649 | N-Biph | N-Biph | H | b1 | H |
| 2-5-650 | N-Biph | N-Biph | t-bu | b1 | H |
| 2-5-651 | N-Biph | N-Biph | methyl | b1 | H |
| 2-5-652 | N-Biph | N-Biph | b1 | b1 | H |
| 2-5-653 | N-Biph | N-Biph | b2 | b1 | H |
| 2-5-654 | N-Biph | N-Biph | b3 | b1 | H |
| 2-5-655 | N-Biph | N-Biph | H | b2 | H |
| 2-5-656 | N-Biph | N-Biph | t-bu | b2 | H |
| 2-5-657 | N-Biph | N-Biph | methyl | b2 | H |
| 2-5-658 | N-Biph | N-Biph | b1 | b2 | H |
| 2-5-659 | N-Biph | N-Biph | b2 | b2 | H |
| 2-5-660 | N-Biph | N-Biph | b3 | b2 | H |
| 2-5-661 | N-Biph | N-Biph | H | b3 | H |
| 2-5-662 | N-Biph | N-Biph | t-bu | b3 | H |
| 2-5-663 | N-Biph | N-Biph | methyl | b3 | H |
| 2-5-664 | N-Biph | N-Biph | b1 | b3 | H |
| 2-5-665 | N-Biph | N-Biph | b2 | b3 | H |
| 2-5-666 | N-Biph | N-Biph | b3 | b3 | H |
| 2-5-667 | N-Biph | N-Biph | H | b4 | H |
| 2-5-668 | N-Biph | N-Biph | t-bu | b4 | H |
| 2-5-669 | N-Biph | N-Biph | methyl | b4 | H |
| 2-5-670 | N-Biph | N-Biph | b1 | b4 | H |
| 2-5-671 | N-Biph | N-Biph | b2 | b4 | H |
| 2-5-672 | N-Biph | N-Biph | b3 | b4 | H |
| 2-5-673 | N-Biph | N-Biph | H | b5 | H |
| 2-5-674 | N-Biph | N-Biph | t-bu | b5 | H |
| 2-5-675 | N-Biph | N-Biph | methyl | b5 | H |
| 2-5-676 | N-Biph | N-Biph | b1 | b5 | H |
| 2-5-677 | N-Biph | N-Biph | b2 | b5 | H |
| 2-5-678 | N-Biph | N-Biph | b3 | b5 | H |
| 2-5-679 | N-Biph | N-Biph | H | b6 | H |
| 2-5-680 | N-Biph | N-Biph | t-bu | b6 | H |
| 2-5-681 | N-Biph | N-Biph | methyl | b6 | H |
| 2-5-682 | N-Biph | N-Biph | b1 | b6 | H |
| 2-5-683 | N-Biph | N-Biph | b2 | b6 | H |
| 2-5-684 | N-Biph | N-Biph | b3 | b6 | H |
| 2-5-685 | N-Biph | N-Biph | H | b7 | H |
| 2-5-686 | N-Biph | N-Biph | t-bu | b7 | H |
| 2-5-687 | N-Biph | N-Biph | methyl | b7 | H |
| 2-5-688 | N-Biph | N-Biph | b1 | b7 | H |
| 2-5-689 | N-Biph | N-Biph | b2 | b7 | H |
| 2-5-690 | N-Biph | N-Biph | b3 | b7 | H |
| 2-5-691 | N-Biph | N-Biph | H | b8 | H |
| 2-5-692 | N-Biph | N-Biph | t-bu | b8 | H |
| 2-5-693 | N-Biph | N-Biph | methyl | b8 | H |
| 2-5-694 | N-Biph | N-Biph | b1 | b8 | H |
| 2-5-695 | N-Biph | N-Biph | b2 | b8 | H |
| 2-5-696 | N-Biph | N-Biph | b3 | b8 | H |
| 2-5-697 | N-Biph | N-Biph | H | b9 | H |
| 2-5-698 | N-Biph | N-Biph | t-bu | b9 | H |
| 2-5-699 | N-Biph | N-Biph | methyl | b9 | H |
| 2-5-700 | N-Biph | N-Biph | b1 | b9 | H |
| 2-5-701 | N-Biph | N-Biph | b2 | b9 | H |
| 2-5-702 | N-Biph | N-Biph | b3 | b9 | H |
| 2-5-703 | N-Biph | N-Biph | H | b10 | H |
| 2-5-704 | N-Biph | N-Biph | t-bu | b10 | H |
| 2-5-705 | N-Biph | N-Biph | methyl | b10 | H |
| 2-5-706 | N-Biph | N-Biph | b1 | b10 | H |
| 2-5-707 | N-Biph | N-Biph | b2 | b10 | H |
| 2-5-708 | N-Biph | N-Biph | b3 | b10 | H |
| 2-5-709 | N-Biph | N-Biph | H | a1 | a1 |
| 2-5-710 | N-Biph | N-Biph | t-bu | a1 | a1 |
| 2-5-711 | N-Biph | N-Biph | methyl | a1 | a1 |
| 2-5-712 | N-Biph | N-Biph | b1 | a1 | a1 |
| 2-5-713 | N-Biph | N-Biph | b2 | a1 | a1 |
| 2-5-714 | N-Biph | N-Biph | b3 | a1 | a1 |
| 2-5-715 | N-Biph | N-Biph | H | a2 | a2 |
| 2-5-716 | N-Biph | N-Biph | t-bu | a2 | a2 |
| 2-5-717 | N-Biph | N-Biph | methyl | a2 | a2 |
| 2-5-718 | N-Biph | N-Biph | b1 | a2 | a2 |
| 2-5-719 | N-Biph | N-Biph | b2 | a2 | a2 |
| 2-5-720 | N-Biph | N-Biph | b3 | a2 | a2 |
| 2-5-721 | N-Biph | N-Biph | H | a3 | a3 |
| 2-5-722 | N-Biph | N-Biph | t-bu | a3 | a3 |
| 2-5-723 | N-Biph | N-Biph | methyl | a3 | a3 |
| 2-5-724 | N-Biph | N-Biph | b1 | a3 | a3 |
| 2-5-725 | N-Biph | N-Biph | b2 | a3 | a3 |
| 2-5-726 | N-Biph | N-Biph | b3 | a3 | a3 |
| 2-5-727 | N-Biph | N-Biph | H | b1 | b1 |
| 2-5-728 | N-Biph | N-Biph | t-bu | b1 | b1 |
| 2-5-729 | N-Biph | N-Biph | methyl | b1 | b1 |
| 2-5-730 | N-Biph | N-Biph | b1 | b1 | b1 |
| 2-5-731 | N-Biph | N-Biph | b2 | b1 | b1 |
| 2-5-732 | N-Biph | N-Biph | b3 | b1 | b1 |
| 2-5-733 | N-Biph | N-Biph | H | b2 | b2 |
| 2-5-734 | N-Biph | N-Biph | t-bu | b2 | b2 |
| 2-5-735 | N-Biph | N-Biph | methyl | b2 | b2 |
| 2-5-736 | N-Biph | N-Biph | b1 | b2 | b2 |
| 2-5-737 | N-Biph | N-Biph | b2 | b2 | b2 |
| 2-5-738 | N-Biph | N-Biph | b3 | b2 | b2 |
| 2-5-739 | N-Biph | N-Biph | H | b3 | b3 |
| 2-5-740 | N-Biph | N-Biph | t-bu | b3 | b3 |
| 2-5-741 | N-Biph | N-Biph | methyl | b3 | b3 |
| 2-5-742 | N-Biph | N-Biph | b1 | b3 | b3 |
| 2-5-743 | N-Biph | N-Biph | b2 | b3 | b3 |
| 2-5-744 | N-Biph | N-Biph | b3 | b3 | b3 |
| 2-5-745 | N-Biph | N-Biph | H | b5 | b5 |
| 2-5-746 | N-Biph | N-Biph | t-bu | b5 | b5 |
| 2-5-747 | N-Biph | N-Biph | methyl | b5 | b5 |
| 2-5-748 | N-Biph | N-Biph | b1 | b5 | b5 |
| 2-5-749 | N-Biph | N-Biph | b2 | b5 | b5 |
| 2-5-750 | N-Biph | N-Biph | b3 | b5 | b5 |
| 2-5-751 | N-Biph | N-Biph | H | b6 | b6 |
| 2-5-752 | N-Biph | N-Biph | t-bu | b6 | b6 |
| 2-5-753 | N-Biph | N-Biph | methyl | b6 | b6 |
| 2-5-754 | N-Biph | N-Biph | b1 | b6 | b6 |
| 2-5-755 | N-Biph | N-Biph | b2 | b6 | b6 |
| 2-5-756 | N-Biph | N-Biph | b3 | b6 | b6 |
| 2-5-757 | N-Biph | N-Biph | H | b7 | b7 |
| 2-5-758 | N-Biph | N-Biph | t-bu | b7 | b7 |
| 2-5-759 | N-Biph | N-Biph | methyl | b7 | b7 |
| 2-5-760 | N-Biph | N-Biph | b1 | b7 | b7 |
| 2-5-761 | N-Biph | N-Biph | b2 | b7 | b7 |
| 2-5-762 | N-Biph | N-Biph | b3 | b7 | b7 |
| 2-5-763 | O | N-Ph | H | H | H |
| 2-5-764 | O | N-Ph | t-bu | H | H |
| 2-5-765 | O | N-Ph | methyl | H | H |
| 2-5-766 | O | N-Ph | t-bu | t-bu | t-bu |
| 2-5-767 | O | N-Ph | methyl | methyl | methyl |
| 2-5-768 | O | N-Ph | b1 | H | H |
| 2-5-769 | O | N-Ph | b2 | H | H |
| 2-5-770 | O | N-Ph | b3 | H | H |
| 2-5-771 | O | N-Ph | H | H | a1 |
| 2-5-772 | O | N-Ph | t-bu | H | a1 |
| 2-5-773 | O | N-Ph | methyl | H | a1 |
| 2-5-774 | O | N-Ph | b1 | H | a1 |
| 2-5-775 | O | N-Ph | b2 | H | a1 |
| 2-5-776 | O | N-Ph | b3 | H | a1 |
| 2-5-777 | O | N-Ph | H | H | a2 |
| 2-5-778 | O | N-Ph | t-bu | H | a2 |
| 2-5-779 | O | N-Ph | methyl | H | a2 |
| 2-5-780 | O | N-Ph | b1 | H | a2 |
| 2-5-781 | O | N-Ph | b2 | H | a2 |
| 2-5-782 | O | N-Ph | b3 | H | a2 |
| 2-5-783 | O | N-Ph | H | H | a3 |
| 2-5-784 | O | N-Ph | t-bu | H | a3 |
| 2-5-785 | O | N-Ph | methyl | H | a3 |
| 2-5-786 | O | N-Ph | b1 | H | a3 |
| 2-5-787 | O | N-Ph | b2 | H | a3 |
| 2-5-788 | O | N-Ph | b3 | H | a3 |
| 2-5-789 | O | N-Ph | H | H | a4 |
| 2-5-790 | O | N-Ph | t-bu | H | a4 |
| 2-5-791 | O | N-Ph | methyl | H | a4 |
| 2-5-792 | O | N-Ph | b1 | H | a4 |
| 2-5-793 | O | N-Ph | b2 | H | a4 |
| 2-5-794 | O | N-Ph | b3 | H | a4 |
| 2-5-795 | O | N-Ph | H | H | a5 |
| 2-5-796 | O | N-Ph | t-bu | H | a5 |
| 2-5-797 | O | N-Ph | methyl | H | a5 |
| 2-5-798 | O | N-Ph | b1 | H | a5 |
| 2-5-799 | O | N-Ph | b2 | H | a5 |
| 2-5-800 | O | N-Ph | b3 | H | a5 |
| 2-5-801 | O | N-Ph | H | H | a6 |
| 2-5-802 | O | N-Ph | t-bu | H | a6 |
| 2-5-803 | O | N-Ph | methyl | H | a6 |
| 2-5-804 | O | N-Ph | b1 | H | a6 |
| 2-5-805 | O | N-Ph | b2 | H | a6 |
| 2-5-806 | O | N-Ph | b3 | H | a6 |
| 2-5-807 | O | N-Ph | H | H | b1 |
| 2-5-808 | O | N-Ph | t-bu | H | b1 |
| 2-5-809 | O | N-Ph | methyl | H | b1 |
| 2-5-810 | O | N-Ph | b1 | H | b1 |
| 2-5-811 | O | N-Ph | b2 | H | b1 |
| 2-5-812 | O | N-Ph | b3 | H | b1 |
| 2-5-813 | O | N-Ph | H | H | b2 |
| 2-5-814 | O | N-Ph | t-bu | H | b2 |
| 2-5-815 | O | N-Ph | methyl | H | b2 |
| 2-5-816 | O | N-Ph | b1 | H | b2 |
| 2-5-817 | O | N-Ph | b2 | H | b2 |
| 2-5-818 | O | N-Ph | b3 | H | b2 |
| 2-5-819 | O | N-Ph | H | H | b3 |
| 2-5-820 | O | N-Ph | t-bu | H | b3 |
| 2-5-821 | O | N-Ph | methyl | H | b3 |
| 2-5-822 | O | N-Ph | b1 | H | b3 |
| 2-5-823 | O | N-Ph | b2 | H | b3 |
| 2-5-824 | O | N-Ph | b3 | H | b3 |
| 2-5-825 | O | N-Ph | H | H | b4 |
| 2-5-826 | O | N-Ph | t-bu | H | b4 |
| 2-5-827 | O | N-Ph | methyl | H | b4 |
| 2-5-828 | O | N-Ph | b1 | H | b4 |
| 2-5-829 | O | N-Ph | b2 | H | b4 |
| 2-5-830 | O | N-Ph | b3 | H | b4 |
| 2-5-831 | O | N-Ph | H | H | b5 |
| 2-5-832 | O | N-Ph | t-bu | H | b5 |
| 2-5-833 | O | N-Ph | methyl | H | b5 |
| 2-5-834 | O | N-Ph | b1 | H | b5 |
| 2-5-835 | O | N-Ph | b2 | H | b5 |
| 2-5-836 | O | N-Ph | b3 | H | b5 |
| 2-5-837 | O | N-Ph | H | H | b6 |
| 2-5-838 | O | N-Ph | t-bu | H | b6 |
| 2-5-839 | O | N-Ph | methyl | H | b6 |
| 2-5-840 | O | N-Ph | b1 | H | b6 |
| 2-5-841 | O | N-Ph | b2 | H | b6 |
| 2-5-842 | O | N-Ph | b3 | H | b6 |
| 2-5-843 | O | N-Ph | H | H | b7 |
| 2-5-844 | O | N-Ph | t-bu | H | b7 |
| 2-5-845 | O | N-Ph | methyl | H | b7 |
| 2-5-846 | O | N-Ph | b1 | H | b7 |
| 2-5-847 | O | N-Ph | b2 | H | b7 |
| 2-5-848 | O | N-Ph | b3 | H | b7 |
| 2-5-849 | O | N-Ph | H | H | b8 |
| 2-5-850 | O | N-Ph | t-bu | H | b8 |
| 2-5-851 | O | N-Ph | methyl | H | b8 |
| 2-5-852 | O | N-Ph | b1 | H | b8 |
| 2-5-853 | O | N-Ph | b2 | H | b8 |
| 2-5-854 | O | N-Ph | b3 | H | b8 |
| 2-5-855 | O | N-Ph | H | H | b9 |
| 2-5-856 | O | N-Ph | t-bu | H | b9 |
| 2-5-857 | O | N-Ph | methyl | H | b9 |
| 2-5-858 | O | N-Ph | b1 | H | b9 |
| 2-5-859 | O | N-Ph | b2 | H | b9 |
| 2-5-860 | O | N-Ph | b3 | H | b9 |
| 2-5-861 | O | N-Ph | H | H | b10 |
| 2-5-862 | O | N-Ph | t-bu | H | b10 |
| 2-5-863 | O | N-Ph | methyl | H | b10 |
| 2-5-864 | O | N-Ph | b1 | H | b10 |
| 2-5-865 | O | N-Ph | b2 | H | b10 |
| 2-5-866 | O | N-Ph | b3 | H | b10 |
| 2-5-867 | O | N-Ph | H | a1 | H |
| 2-5-868 | O | N-Ph | t-bu | a1 | H |
| 2-5-869 | O | N-Ph | methyl | a1 | H |
| 2-5-870 | O | N-Ph | b1 | a1 | H |
| 2-5-871 | O | N-Ph | b2 | a1 | H |
| 2-5-872 | O | N-Ph | b3 | a1 | H |
| 2-5-873 | O | N-Ph | H | a2 | H |
| 2-5-874 | O | N-Ph | t-bu | a2 | H |
| 2-5-875 | O | N-Ph | methyl | a2 | H |
| 2-5-876 | O | N-Ph | b1 | a2 | H |
| 2-5-877 | O | N-Ph | b2 | a2 | H |
| 2-5-878 | O | N-Ph | b3 | a2 | H |
| 2-5-879 | O | N-Ph | H | a3 | H |
| 2-5-880 | O | N-Ph | t-bu | a3 | H |
| 2-5-881 | O | N-Ph | methyl | a3 | H |
| 2-5-882 | O | N-Ph | b1 | a3 | H |
| 2-5-883 | O | N-Ph | b2 | a3 | H |
| 2-5-884 | O | N-Ph | b3 | a3 | H |
| 2-5-885 | O | N-Ph | H | a4 | H |
| 2-5-886 | O | N-Ph | t-bu | a4 | H |
| 2-5-887 | O | N-Ph | methyl | a4 | H |
| 2-5-888 | O | N-Ph | b1 | a4 | H |
| 2-5-889 | O | N-Ph | b2 | a4 | H |
| 2-5-890 | O | N-Ph | b3 | a4 | H |
| 2-5-891 | O | N-Ph | H | a5 | H |
| 2-5-892 | O | N-Ph | t-bu | a5 | H |
| 2-5-893 | O | N-Ph | methyl | a5 | H |
| 2-5-894 | O | N-Ph | b1 | a5 | H |
| 2-5-895 | O | N-Ph | b2 | a5 | H |
| 2-5-896 | O | N-Ph | b3 | a5 | H |
| 2-5-897 | O | N-Ph | H | a6 | H |
| 2-5-898 | O | N-Ph | t-bu | a6 | H |
| 2-5-899 | O | N-Ph | methyl | a6 | H |
| 2-5-900 | O | N-Ph | b1 | a6 | H |
| 2-5-901 | O | N-Ph | b2 | a6 | H |
| 2-5-902 | O | N-Ph | b3 | a6 | H |
| 2-5-903 | O | N-Ph | H | b1 | H |
| 2-5-904 | O | N-Ph | t-bu | b1 | H |
| 2-5-905 | O | N-Ph | methyl | b1 | H |
| 2-5-906 | O | N-Ph | b1 | b1 | H |
| 2-5-907 | O | N-Ph | b2 | b1 | H |
| 2-5-908 | O | N-Ph | b3 | b1 | H |
| 2-5-909 | O | N-Ph | H | b2 | H |
| 2-5-910 | O | N-Ph | t-bu | b2 | H |
| 2-5-911 | O | N-Ph | methyl | b2 | H |
| 2-5-912 | O | N-Ph | b1 | b2 | H |
| 2-5-913 | O | N-Ph | b2 | b2 | H |
| 2-5-914 | O | N-Ph | b3 | b2 | H |
| 2-5-915 | O | N-Ph | H | b3 | H |
| 2-5-916 | O | N-Ph | t-bu | b3 | H |
| 2-5-917 | O | N-Ph | methyl | b3 | H |
| 2-5-918 | O | N-Ph | b1 | b3 | H |
| 2-5-919 | O | N-Ph | b2 | b3 | H |
| 2-5-920 | O | N-Ph | b3 | b3 | H |
| 2-5-921 | O | N-Ph | H | b4 | H |
| 2-5-922 | O | N-Ph | t-bu | b4 | H |
| 2-5-923 | O | N-Ph | methyl | b4 | H |
| 2-5-924 | O | N-Ph | b1 | b4 | H |
| 2-5-925 | O | N-Ph | b2 | b4 | H |
| 2-5-926 | O | N-Ph | b3 | b4 | H |
| 2-5-927 | O | N-Ph | H | b5 | H |
| 2-5-928 | O | N-Ph | t-bu | b5 | H |
| 2-5-929 | O | N-Ph | methyl | b5 | H |
| 2-5-930 | O | N-Ph | b1 | b5 | H |
| 2-5-931 | O | N-Ph | b2 | b5 | H |
| 2-5-932 | O | N-Ph | b3 | b5 | H |
| 2-5-933 | O | N-Ph | H | b6 | H |
| 2-5-934 | O | N-Ph | t-bu | b6 | H |
| 2-5-935 | O | N-Ph | methyl | b6 | H |
| 2-5-936 | O | N-Ph | b1 | b6 | H |
| 2-5-937 | O | N-Ph | b2 | b6 | H |
| 2-5-938 | O | N-Ph | b3 | b6 | H |
| 2-5-939 | O | N-Ph | H | b7 | H |
| 2-5-940 | O | N-Ph | t-bu | b7 | H |
| 2-5-941 | O | N-Ph | methyl | b7 | H |
| 2-5-942 | O | N-Ph | b1 | b7 | H |
| 2-5-943 | O | N-Ph | b2 | b7 | H |
| 2-5-944 | O | N-Ph | b3 | b7 | H |
| 2-5-945 | O | N-Ph | H | b8 | H |
| 2-5-946 | O | N-Ph | t-bu | b8 | H |
| 2-5-947 | O | N-Ph | methyl | b8 | H |
| 2-5-948 | O | N-Ph | b1 | b8 | H |
| 2-5-949 | O | N-Ph | b2 | b8 | H |
| 2-5-950 | O | N-Ph | b3 | b8 | H |
| 2-5-951 | O | N-Ph | H | b9 | H |
| 2-5-952 | O | N-Ph | t-bu | b9 | H |
| 2-5-953 | O | N-Ph | methyl | b9 | H |
| 2-5-954 | O | N-Ph | b1 | b9 | H |
| 2-5-955 | O | N-Ph | b2 | b9 | H |
| 2-5-956 | O | N-Ph | b3 | b9 | H |
| 2-5-957 | O | N-Ph | H | b10 | H |
| 2-5-958 | O | N-Ph | t-bu | b10 | H |
| 2-5-959 | O | N-Ph | methyl | b10 | H |
| 2-5-960 | O | N-Ph | b1 | b10 | H |
| 2-5-961 | O | N-Ph | b2 | b10 | H |
| 2-5-962 | O | N-Ph | b3 | b10 | H |
| 2-5-963 | O | N-Ph | H | a1 | a1 |
| 2-5-964 | O | N-Ph | t-bu | a1 | a1 |
| 2-5-965 | O | N-Ph | methyl | a1 | a1 |
| 2-5-966 | O | N-Ph | b1 | a1 | a1 |
| 2-5-967 | O | N-Ph | b2 | a1 | a1 |
| 2-5-968 | O | N-Ph | b3 | a1 | a1 |
| 2-5-969 | O | N-Ph | H | a2 | a2 |
| 2-5-970 | O | N-Ph | t-bu | a2 | a2 |
| 2-5-971 | O | N-Ph | methyl | a2 | a2 |
| 2-5-972 | O | N-Ph | b1 | a2 | a2 |
| 2-5-973 | O | N-Ph | b2 | a2 | a2 |
| 2-5-974 | O | N-Ph | b3 | a2 | a2 |
| 2-5-975 | O | N-Ph | H | a3 | a3 |
| 2-5-976 | O | N-Ph | t-bu | a3 | a3 |
| 2-5-977 | O | N-Ph | methyl | a3 | a3 |
| 2-5-978 | O | N-Ph | b1 | a3 | a3 |
| 2-5-979 | O | N-Ph | b2 | a3 | a3 |
| 2-5-980 | O | N-Ph | b3 | a3 | a3 |
| 2-5-981 | O | N-Ph | H | b1 | b1 |
| 2-5-982 | O | N-Ph | t-bu | b1 | b1 |
| 2-5-983 | O | N-Ph | methyl | b1 | b1 |
| 2-5-984 | O | N-Ph | b1 | b1 | b1 |
| 2-5-985 | O | N-Ph | b2 | b1 | b1 |
| 2-5-986 | O | N-Ph | b3 | b1 | b1 |
| 2-5-987 | O | N-Ph | H | b2 | b2 |
| 2-5-988 | O | N-Ph | t-bu | b2 | b2 |
| 2-5-989 | O | N-Ph | methyl | b2 | b2 |
| 2-5-990 | O | N-Ph | b1 | b2 | b2 |
| 2-5-991 | O | N-Ph | b2 | b2 | b2 |
| 2-5-992 | O | N-Ph | b3 | b2 | b2 |
| 2-5-993 | O | N-Ph | H | b3 | b3 |
| 2-5-994 | O | N-Ph | t-bu | b3 | b3 |
| 2-5-995 | O | N-Ph | methyl | b3 | b3 |
| 2-5-996 | O | N-Ph | b1 | b3 | b3 |
| 2-5-997 | O | N-Ph | b2 | b3 | b3 |
| 2-5-998 | O | N-Ph | b3 | b3 | b3 |
| 2-5-999 | O | N-Ph | H | b5 | b5 |
| 2-5-1000 | O | N-Ph | t-bu | b5 | b5 |
| 2-5-1001 | O | N-Ph | methyl | b5 | b5 |
| 2-5-1002 | O | N-Ph | b1 | b5 | b5 |
| 2-5-1003 | O | N-Ph | b2 | b5 | b5 |
| 2-5-1004 | O | N-Ph | b3 | b5 | b5 |
| 2-5-1005 | O | N-Ph | H | b6 | b6 |
| 2-5-1006 | O | N-Ph | t-bu | b6 | b6 |
| 2-5-1007 | O | N-Ph | methyl | b6 | b6 |
| 2-5-1008 | O | N-Ph | b1 | b6 | b6 |
| 2-5-1009 | O | N-Ph | b2 | b6 | b6 |
| 2-5-1010 | O | N-Ph | b3 | b6 | b6 |
| 2-5-1011 | O | N-Ph | H | b7 | b7 |
| 2-5-1012 | O | N-Ph | t-bu | b7 | b7 |
| 2-5-1013 | O | N-Ph | methyl | b7 | b7 |
| 2-5-1014 | O | N-Ph | b1 | b7 | b7 |
| 2-5-1015 | O | N-Ph | b2 | b7 | b7 |
| 2-5-1016 | O | N-Ph | b3 | b7 | b7 |
| 2-5-1017 | S | N-Ph | H | H | H |
| 2-5-1018 | S | N-Ph | t-bu | H | H |
| 2-5-1019 | S | N-Ph | methyl | H | H |
| 2-5-1020 | S | N-Ph | t-bu | t-bu | t-bu |
| 2-5-1021 | S | N-Ph | methyl | methyl | methyl |
| 2-5-1022 | S | N-Ph | b1 | H | H |
| 2-5-1023 | S | N-Ph | b2 | H | H |
| 2-5-1024 | S | N-Ph | b3 | H | H |
| 2-5-1025 | S | N-Ph | H | H | a1 |
| 2-5-1026 | S | N-Ph | t-bu | H | a1 |
| 2-5-1027 | S | N-Ph | methyl | H | a1 |
| 2-5-1028 | S | N-Ph | b1 | H | a1 |
| 2-5-1029 | S | N-Ph | b2 | H | a1 |
| 2-5-1030 | S | N-Ph | b3 | H | a1 |
| 2-5-1031 | S | N-Ph | H | H | a2 |
| 2-5-1032 | S | N-Ph | t-bu | H | a2 |
| 2-5-1033 | S | N-Ph | methyl | H | a2 |
| 2-5-1034 | S | N-Ph | b1 | H | a2 |
| 2-5-1035 | S | N-Ph | b2 | H | a2 |
| 2-5-1036 | S | N-Ph | b3 | H | a2 |
| 2-5-1037 | S | N-Ph | H | H | a3 |
| 2-5-1038 | S | N-Ph | t-bu | H | a3 |
| 2-5-1039 | S | N-Ph | methyl | H | a3 |
| 2-5-1040 | S | N-Ph | b1 | H | a3 |
| 2-5-1041 | S | N-Ph | b2 | H | a3 |
| 2-5-1042 | S | N-Ph | b3 | H | a3 |
| 2-5-1043 | S | N-Ph | H | H | a4 |
| 2-5-1044 | S | N-Ph | t-bu | H | a4 |
| 2-5-1045 | S | N-Ph | methyl | H | a4 |
| 2-5-1046 | S | N-Ph | b1 | H | a4 |
| 2-5-1047 | S | N-Ph | b2 | H | a4 |
| 2-5-1048 | S | N-Ph | b3 | H | a4 |
| 2-5-1049 | S | N-Ph | H | H | a5 |
| 2-5-1050 | S | N-Ph | t-bu | H | a5 |
| 2-5-1051 | S | N-Ph | methyl | H | a5 |
| 2-5-1052 | S | N-Ph | b1 | H | a5 |
| 2-5-1053 | S | N-Ph | b2 | H | a5 |
| 2-5-1054 | S | N-Ph | b3 | H | a5 |
| 2-5-1055 | S | N-Ph | H | H | a6 |
| 2-5-1056 | S | N-Ph | t-bu | H | a6 |
| 2-5-1057 | S | N-Ph | methyl | H | a6 |
| 2-5-1058 | S | N-Ph | b1 | H | a6 |
| 2-5-1059 | S | N-Ph | b2 | H | a6 |
| 2-5-1060 | S | N-Ph | b3 | H | a6 |
| 2-5-1061 | S | N-Ph | H | H | b1 |
| 2-5-1062 | S | N-Ph | t-bu | H | b1 |
| 2-5-1063 | S | N-Ph | methyl | H | b1 |
| 2-5-1064 | S | N-Ph | b1 | H | b1 |
| 2-5-1065 | S | N-Ph | b2 | H | b1 |
| 2-5-1066 | S | N-Ph | b3 | H | b1 |
| 2-5-1067 | S | N-Ph | H | H | b2 |
| 2-5-1068 | S | N-Ph | t-bu | H | b2 |
| 2-5-1069 | S | N-Ph | methyl | H | b2 |
| 2-5-1070 | S | N-Ph | b1 | H | b2 |
| 2-5-1071 | S | N-Ph | b2 | H | b2 |
| 2-5-1072 | S | N-Ph | b3 | H | b2 |
| 2-5-1073 | S | N-Ph | H | H | b3 |
| 2-5-1074 | S | N-Ph | t-bu | H | b3 |
| 2-5-1075 | S | N-Ph | methyl | H | b3 |
| 2-5-1076 | S | N-Ph | b1 | H | b3 |
| 2-5-1077 | S | N-Ph | b2 | H | b3 |
| 2-5-1078 | S | N-Ph | b3 | H | b3 |
| 2-5-1079 | S | N-Ph | H | H | b4 |
| 2-5-1080 | S | N-Ph | t-bu | H | b4 |
| 2-5-1081 | S | N-Ph | methyl | H | b4 |
| 2-5-1082 | S | N-Ph | b1 | H | b4 |
| 2-5-1083 | S | N-Ph | b2 | H | b4 |
| 2-5-1084 | S | N-Ph | b3 | H | b4 |
| 2-5-1085 | S | N-Ph | H | H | b5 |
| 2-5-1086 | S | N-Ph | t-bu | H | b5 |
| 2-5-1087 | S | N-Ph | methyl | H | b5 |
| 2-5-1088 | S | N-Ph | b1 | H | b5 |
| 2-5-1089 | S | N-Ph | b2 | H | b5 |
| 2-5-1090 | S | N-Ph | b3 | H | b5 |
| 2-5-1091 | S | N-Ph | H | H | b6 |
| 2-5-1092 | S | N-Ph | t-bu | H | b6 |
| 2-5-1093 | S | N-Ph | methyl | H | b6 |
| 2-5-1094 | S | N-Ph | b1 | H | b6 |
| 2-5-1095 | S | N-Ph | b2 | H | b6 |
| 2-5-1096 | S | N-Ph | b3 | H | b6 |
| 2-5-1097 | S | N-Ph | H | H | b7 |
| 2-5-1098 | S | N-Ph | t-bu | H | b7 |
| 2-5-1099 | S | N-Ph | methyl | H | b7 |
| 2-5-1100 | S | N-Ph | b1 | H | b7 |
| 2-5-1101 | S | N-Ph | b2 | H | b7 |
| 2-5-1102 | S | N-Ph | b3 | H | b7 |
| 2-5-1103 | S | N-Ph | H | H | b8 |
| 2-5-1104 | S | N-Ph | t-bu | H | b8 |
| 2-5-1105 | S | N-Ph | methyl | H | b8 |
| 2-5-1106 | S | N-Ph | b1 | H | b8 |
| 2-5-1107 | S | N-Ph | b2 | H | b8 |
| 2-5-1108 | S | N-Ph | b3 | H | b8 |
| 2-5-1109 | S | N-Ph | H | H | b9 |
| 2-5-1110 | S | N-Ph | t-bu | H | b9 |
| 2-5-1111 | S | N-Ph | methyl | H | b9 |
| 2-5-1112 | S | N-Ph | b1 | H | b9 |
| 2-5-1113 | S | N-Ph | b2 | H | b9 |
| 2-5-1114 | S | N-Ph | b3 | H | b9 |
| 2-5-1115 | S | N-Ph | H | H | b10 |
| 2-5-1116 | S | N-Ph | t-bu | H | b10 |
| 2-5-1117 | S | N-Ph | methyl | H | b10 |
| 2-5-1118 | S | N-Ph | b1 | H | b10 |
| 2-5-1119 | S | N-Ph | b2 | H | b10 |
| 2-5-1120 | S | N-Ph | b3 | H | b10 |
| 2-5-1121 | S | N-Ph | H | a1 | H |
| 2-5-1122 | S | N-Ph | t-bu | a1 | H |
| 2-5-1123 | S | N-Ph | methyl | a1 | H |
| 2-5-1124 | S | N-Ph | b1 | a1 | H |
| 2-5-1125 | S | N-Ph | b2 | a1 | H |
| 2-5-1126 | S | N-Ph | b3 | a1 | H |
| 2-5-1127 | S | N-Ph | H | a2 | H |
| 2-5-1128 | S | N-Ph | t-bu | a2 | H |
| 2-5-1129 | S | N-Ph | methyl | a2 | H |
| 2-5-1130 | S | N-Ph | b1 | a2 | H |
| 2-5-1131 | S | N-Ph | b2 | a2 | H |
| 2-5-1132 | S | N-Ph | b3 | a2 | H |
| 2-5-1133 | S | N-Ph | H | a3 | H |
| 2-5-1134 | S | N-Ph | t-bu | a3 | H |
| 2-5-1135 | S | N-Ph | methyl | a3 | H |
| 2-5-1136 | S | N-Ph | b1 | a3 | H |
| 2-5-1137 | S | N-Ph | b2 | a3 | H |
| 2-5-1138 | S | N-Ph | b3 | a3 | H |
| 2-5-1139 | S | N-Ph | H | a4 | H |
| 2-5-1140 | S | N-Ph | t-bu | a4 | H |
| 2-5-1141 | S | N-Ph | methyl | a4 | H |
| 2-5-1142 | S | N-Ph | b1 | a4 | H |
| 2-5-1143 | S | N-Ph | b2 | a4 | H |
| 2-5-1144 | S | N-Ph | b3 | a4 | H |
| 2-5-1145 | S | N-Ph | H | a5 | H |
| 2-5-1146 | S | N-Ph | t-bu | a5 | H |
| 2-5-1147 | S | N-Ph | methyl | a5 | H |
| 2-5-1148 | S | N-Ph | b1 | a5 | H |
| 2-5-1149 | S | N-Ph | b2 | a5 | H |
| 2-5-1150 | S | N-Ph | b3 | a5 | H |
| 2-5-1151 | S | N-Ph | H | a6 | H |
| 2-5-1152 | S | N-Ph | t-bu | a6 | H |
| 2-5-1153 | S | N-Ph | methyl | a6 | H |
| 2-5-1154 | S | N-Ph | b1 | a6 | H |
| 2-5-1155 | S | N-Ph | b2 | a6 | H |
| 2-5-1156 | S | N-Ph | b3 | a6 | H |
| 2-5-1157 | S | N-Ph | H | b1 | H |
| 2-5-1158 | S | N-Ph | t-bu | b1 | H |
| 2-5-1159 | S | N-Ph | methyl | b1 | H |
| 2-5-1160 | S | N-Ph | b1 | b1 | H |
| 2-5-1161 | S | N-Ph | b2 | b1 | H |
| 2-5-1162 | S | N-Ph | b3 | b1 | H |
| 2-5-1163 | S | N-Ph | H | b2 | H |
| 2-5-1164 | S | N-Ph | t-bu | b2 | H |
| 2-5-1165 | S | N-Ph | methyl | b2 | H |
| 2-5-1166 | S | N-Ph | b1 | b2 | H |
| 2-5-1167 | S | N-Ph | b2 | b2 | H |
| 2-5-1168 | S | N-Ph | b3 | b2 | H |
| 2-5-1169 | S | N-Ph | H | b3 | H |
| 2-5-1170 | S | N-Ph | t-bu | b3 | H |
| 2-5-1171 | S | N-Ph | methyl | b3 | H |
| 2-5-1172 | S | N-Ph | b1 | b3 | H |
| 2-5-1173 | S | N-Ph | b2 | b3 | H |
| 2-5-1174 | S | N-Ph | b3 | b3 | H |
| 2-5-1175 | S | N-Ph | H | b4 | H |
| 2-5-1176 | S | N-Ph | t-bu | b4 | H |
| 2-5-1177 | S | N-Ph | methyl | b4 | H |
| 2-5-1178 | S | N-Ph | b1 | b4 | H |
| 2-5-1179 | S | N-Ph | b2 | b4 | H |
| 2-5-1180 | S | N-Ph | b3 | b4 | H |
| 2-5-1181 | S | N-Ph | H | b5 | H |
| 2-5-1182 | S | N-Ph | t-bu | b5 | H |
| 2-5-1183 | S | N-Ph | methyl | b5 | H |
| 2-5-1184 | S | N-Ph | b1 | b5 | H |
| 2-5-1185 | S | N-Ph | b2 | b5 | H |
| 2-5-1186 | S | N-Ph | b3 | b5 | H |
| 2-5-1187 | S | N-Ph | H | b6 | H |
| 2-5-1188 | S | N-Ph | t-bu | b6 | H |
| 2-5-1189 | S | N-Ph | methyl | b6 | H |
| 2-5-1190 | S | N-Ph | b1 | b6 | H |
| 2-5-1191 | S | N-Ph | b2 | b6 | H |
| 2-5-1192 | S | N-Ph | b3 | b6 | H |
| 2-5-1193 | S | N-Ph | H | b7 | H |
| 2-5-1194 | S | N-Ph | t-bu | b7 | H |
| 2-5-1195 | S | N-Ph | methyl | b7 | H |
| 2-5-1196 | S | N-Ph | b1 | b7 | H |
| 2-5-1197 | S | N-Ph | b2 | b7 | H |
| 2-5-1198 | S | N-Ph | b3 | b7 | H |
| 2-5-1199 | S | N-Ph | H | b8 | H |
| 2-5-1200 | 5 | N-Ph | t-bu | b8 | H |
| 2-5-1201 | S | N-Ph | methyl | b8 | H |
| 2-5-1202 | S | N-Ph | b1 | b8 | H |
| 2-5-1203 | S | N-Ph | b2 | b8 | H |
| 2-5-1204 | S | N-Ph | b3 | b8 | H |
| 2-5-1205 | S | N-Ph | H | b9 | H |
| 2-5-1206 | S | N-Ph | t-bu | b9 | H |
| 2-5-1207 | S | N-Ph | methyl | b9 | H |
| 2-5-1208 | S | N-Ph | b1 | b9 | H |
| 2-5-1209 | S | N-Ph | b2 | b9 | H |
| 2-5-1210 | S | N-Ph | b3 | b9 | H |
| 2-5-1211 | S | N-Ph | H | b10 | H |
| 2-5-1212 | S | N-Ph | t-bu | b10 | H |
| 2-5-1213 | S | N-Ph | methyl | b10 | H |
| 2-5-1214 | S | N-Ph | b1 | b10 | H |
| 2-5-1215 | S | N-Ph | b2 | b10 | H |
| 2-5-1216 | S | N-Ph | b3 | b10 | H |
| 2-5-1217 | S | N-Ph | H | a1 | a1 |
| 2-5-1218 | S | N-Ph | t-bu | a1 | a1 |
| 2-5-1219 | S | N-Ph | methyl | a1 | a1 |
| 2-5-1220 | S | N-Ph | b1 | a1 | a1 |
| 2-5-1221 | S | N-Ph | b2 | a1 | a1 |
| 2-5-1222 | S | N-Ph | b3 | a1 | a1 |
| 2-5-1223 | S | N-Ph | H | a2 | a2 |
| 2-5-1224 | S | N-Ph | t-bu | a2 | a2 |
| 2-5-1225 | S | N-Ph | methyl | a2 | a2 |
| 2-5-1226 | S | N-Ph | b1 | a2 | a2 |
| 2-5-1227 | S | N-Ph | b2 | a2 | a2 |
| 2-5-1228 | S | N-Ph | b3 | a2 | a2 |
| 2-5-1229 | S | N-Ph | H | a3 | a3 |
| 2-5-1230 | S | N-Ph | t-bu | a3 | a3 |
| 2-5-1231 | S | N-Ph | methyl | a3 | a3 |
| 2-5-1232 | S | N-Ph | b1 | a3 | a3 |
| 2-5-1233 | S | N-Ph | b2 | a3 | a3 |
| 2-5-1234 | S | N-Ph | b3 | a3 | a3 |
| 2-5-1235 | S | N-Ph | H | b1 | b1 |
| 2-5-1236 | S | N-Ph | t-bu | b1 | b1 |
| 2-5-1237 | S | N-Ph | methyl | b1 | b1 |
| 2-5-1238 | S | N-Ph | b1 | b1 | b1 |
| 2-5-1239 | S | N-Ph | b2 | b1 | b1 |
| 2-5-1240 | S | N-Ph | b3 | b1 | b1 |
| 2-5-1241 | S | N-Ph | H | b2 | b2 |
| 2-5-1242 | S | N-Ph | t-bu | b2 | b2 |
| 2-5-1243 | S | N-Ph | methyl | b2 | b2 |
| 2-5-1244 | S | N-Ph | b1 | b2 | b2 |
| 2-5-1245 | S | N-Ph | b2 | b2 | b2 |
| 2-5-1246 | S | N-Ph | b3 | b2 | b2 |
| 2-5-1247 | S | N-Ph | H | b3 | b3 |
| 2-5-1248 | S | N-Ph | t-bu | b3 | b3 |
| 2-5-1249 | S | N-Ph | methyl | b3 | b3 |
| 2-5-1250 | S | N-Ph | b1 | b3 | b3 |
| 2-5-1251 | S | N-Ph | b2 | b3 | b3 |
| 2-5-1252 | S | N-Ph | b3 | b3 | b3 |
| 2-5-1253 | S | N-Ph | H | b5 | b5 |
| 2-5-1254 | S | N-Ph | t-bu | b5 | b5 |
| 2-5-1255 | S | N-Ph | methyl | b5 | b5 |
| 2-5-1256 | S | N-Ph | b1 | b5 | b5 |
| 2-5-1257 | S | N-Ph | b2 | b5 | b5 |
| 2-5-1258 | S | N-Ph | b3 | b5 | b5 |
| 2-5-1259 | S | N-Ph | H | b6 | b6 |
| 2-5-1260 | S | N-Ph | t-bu | b6 | b6 |
| 2-5-1261 | S | N-Ph | methyl | b6 | b6 |
| 2-5-1262 | S | N-Ph | b1 | b6 | b6 |
| 2-5-1263 | S | N-Ph | b2 | b6 | b6 |
| 2-5-1264 | S | N-Ph | b3 | b6 | b6 |
| 2-5-1265 | S | N-Ph | H | b7 | b7 |
| 2-5-1266 | S | N-Ph | t-bu | b7 | b7 |
| 2-5-1267 | S | N-Ph | methyl | b7 | b7 |
| 2-5-1268 | S | N-Ph | b1 | b7 | b7 |
| 2-5-1269 | S | N-Ph | b2 | b7 | b7 |
| 2-5-1270 | S | N-Ph | b3 | b7 | b7 |

Group 2-6
The compounds are represented by the formula 2-6 below, wherein CY₃ is a benzene group, X₁ is B, and Y1, Y2, R1b, R1d, R2b, R2d, (R3)a3 are each shown in the table 6 below, 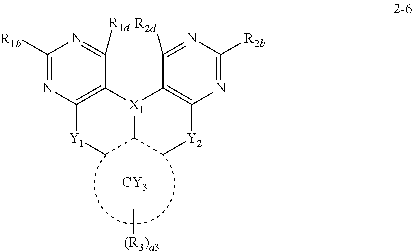
TABLE 6

| Compound | | | | | | | |
|---|---|---|---|---|---|---|---|
| No. | Y1 | Y2 | R1b | R1d | R2b | R2d | (R3)a3 |
| 2-6-001 | N—Ph | N—Ph | H | H | H | H | H |
| 2-6-002 | N—Ph | N—Ph | t-bu | H | t-bu | H | H |
| 2-6-003 | N—Ph | N—Ph | methyl | H | methyl | H | H |
| 2-6-004 | N—Ph | N—Ph | t-bu | t-bu | t-bu | t-bu | t-bu |
| 2-6-005 | N—Ph | N—Ph | methyl | methyl | methyl | methyl | methyl |
| 2-6-006 | N—Ph | N—Ph | b1 | H | b1 | H | H |
| 2-6-007 | N—Ph | N—Ph | b2 | H | b2 | H | H |
| 2-6-008 | N—Ph | N—Ph | b3 | H | b3 | H | H |
| 2-6-009 | N—Ph | N—Ph | H | H | H | H | a1 |
| 2-6-010 | N—Ph | N—Ph | t-bu | H | t-bu | H | a1 |
| 2-6-011 | N—Ph | N—Ph | methyl | H | methyl | H | a1 |
| 2-6-012 | N—Ph | N—Ph | b1 | H | b1 | H | a1 |
| 2-6-013 | N—Ph | N—Ph | b2 | H | b2 | H | a1 |
| 2-6-014 | N—Ph | N—Ph | b3 | H | b3 | H | a1 |
| 2-6-015 | N—Ph | N—Ph | H | H | H | H | a2 |
| 2-6-016 | N—Ph | N—Ph | t-bu | H | t-bu | H | a2 |
| 2-6-017 | N—Ph | N—Ph | methyl | H | methyl | H | a2 |
| 2-6-018 | N—Ph | N—Ph | b1 | H | b1 | H | a2 |
| 2-6-019 | N—Ph | N—Ph | b2 | H | b2 | H | a2 |
| 2-6-020 | N—Ph | N—Ph | b3 | H | b3 | H | a2 |
| 2-6-021 | N—Ph | N—Ph | H | H | H | H | a3 |
| 2-6-022 | N—Ph | N—Ph | t-bu | H | t-bu | H | a3 |
| 2-6-023 | N—Ph | N—Ph | methyl | H | methyl | H | a3 |
| 2-6-024 | N—Ph | N—Ph | b1 | H | b1 | H | a3 |
| 2-6-025 | N—Ph | N—Ph | b2 | H | b2 | H | a3 |
| 2-6-026 | N—Ph | N—Ph | b3 | H | b3 | H | a3 |
| 2-6-027 | N—Ph | N—Ph | H | H | H | H | a4 |
| 2-6-028 | N—Ph | N—Ph | t-bu | H | t-bu | H | a4 |
| 2-6-029 | N—Ph | N—Ph | methyl | H | methyl | H | a4 |
| 2-6-030 | N—Ph | N—Ph | b1 | H | b1 | H | a4 |
| 2-6-031 | N—Ph | N—Ph | b2 | H | b2 | H | a4 |
| 2-6-032 | N—Ph | N—Ph | b3 | H | b3 | H | a4 |
| 2-6-033 | N—Ph | N—Ph | H | H | H | H | a5 |
| 2-6-034 | N—Ph | N—Ph | t-bu | H | t-bu | H | a5 |
| 2-6-035 | N—Ph | N—Ph | methyl | H | methyl | H | a5 |
| 2-6-036 | N—Ph | N—Ph | b1 | H | b1 | H | a5 |
| 2-6-037 | N—Ph | N—Ph | b2 | H | b2 | H | a5 |
| 2-6-038 | N—Ph | N—Ph | b3 | H | b3 | H | a5 |
| 2-6-039 | N—Ph | N—Ph | H | H | H | H | a6 |
| 2-6-040 | N—Ph | N—Ph | t-bu | H | t-bu | H | a6 |
| 2-6-041 | N—Ph | N—Ph | methyl | H | methyl | H | a6 |
| 2-6-042 | N—Ph | N—Ph | b1 | H | b1 | H | a6 |
| 2-6-043 | N—Ph | N—Ph | b2 | H | b2 | H | a6 |
| 2-6-044 | N—Ph | N—Ph | b3 | H | b3 | H | a6 |
| 2-6-045 | N-tolyl | N-tolyl | H | H | H | H | H |
| 2-6-046 | N-tolyl | N-tolyl | t-bu | H | t-bu | H | H |
| 2-6-047 | N-tolyl | N-tolyl | methyl | H | methyl | H | H |
| 2-6-048 | N-tolyl | N-tolyl | t-bu | t-bu | t-bu | t-bu | t-bu |
| 2-6-049 | N-tolyl | N-tolyl | methyl | methyl | methyl | methyl | methyl |
| 2-6-050 | N-tolyl | N-tolyl | b1 | H | b1 | H | H |
| 2-6-051 | N-tolyl | N-tolyl | b2 | H | b2 | H | H |
| 2-6-052 | N-tolyl | N-tolyl | b3 | H | b3 | H | H |
| 2-6-053 | N-tolyl | N-tolyl | H | H | H | H | a1 |
| 2-6-054 | N-tolyl | N-tolyl | t-bu | H | t-bu | H | a1 |
| 2-6-055 | N-tolyl | N-tolyl | methyl | H | methyl | H | a1 |
| 2-6-056 | N-tolyl | N-tolyl | b1 | H | b1 | H | a1 |
| 2-6-057 | N-tolyl | N-tolyl | b2 | H | b2 | H | a1 |
| 2-6-058 | N-tolyl | N-tolyl | b3 | H | b3 | H | a1 |
| 2-6-059 | N-tolyl | N-tolyl | H | H | H | H | a2 |
| 2-6-060 | N-tolyl | N-tolyl | t-bu | H | t-bu | H | a2 |
| 2-6-061 | N-tolyl | N-tolyl | methyl | H | methyl | H | a2 |
| 2-6-062 | N-tolyl | N-tolyl | b1 | H | b1 | H | a2 |
| 2-6-063 | N-tolyl | N-tolyl | b2 | H | b2 | H | a2 |
| 2-6-064 | N-tolyl | N-tolyl | b3 | H | b3 | H | a2 |
| 2-6-065 | N-tolyl | N-tolyl | H | H | H | H | a3 |
| 2-6-066 | N-tolyl | N-tolyl | t-bu | H | t-bu | H | a3 |
| 2-6-067 | N-tolyl | N-tolyl | methyl | H | methyl | H | a3 |
| 2-6-068 | N-tolyl | N-tolyl | b1 | H | b1 | H | a3 |
| 2-6-069 | N-tolyl | N-tolyl | b2 | H | b2 | H | a3 |
| 2-6-070 | N-tolyl | N-tolyl | b3 | H | b3 | H | a3 |
| 2-6-071 | N-tolyl | N-tolyl | H | H | H | H | a4 |
| 2-6-072 | N-tolyl | N-tolyl | t-bu | H | t-bu | H | a4 |
| 2-6-073 | N-tolyl | N-tolyl | methyl | H | methyl | H | a4 |
| 2-6-074 | N-tolyl | N-tolyl | b1 | H | b1 | H | a4 |
| 2-6-075 | N-tolyl | N-tolyl | b2 | H | b2 | H | a4 |
| 2-6-076 | N-tolyl | N-tolyl | b3 | H | b3 | H | a4 |
| 2-6-077 | N-tolyl | N-tolyl | H | H | H | H | a5 |
| 2-6-078 | N-tolyl | N-tolyl | t-bu | H | t-bu | H | a5 |
| 2-6-079 | N-tolyl | N-tolyl | methyl | H | methyl | H | a5 |
| 2-6-080 | N-tolyl | N-tolyl | b1 | H | b1 | H | a5 |
| 2-6-081 | N-tolyl | N-tolyl | b2 | H | b2 | H | a5 |
| 2-6-082 | N-tolyl | N-tolyl | b3 | H | b3 | H | a5 |
| 2-6-083 | N-tolyl | N-tolyl | H | H | H | H | a6 |
| 2-6-084 | N-tolyl | N-tolyl | t-bu | H | t-bu | H | a6 |
| 2-6-085 | N-tolyl | N-tolyl | methyl | H | methyl | H | a6 |
| 2-6-086 | N-tolyl | N-tolyl | b1 | H | b1 | H | a6 |
| 2-6-087 | N-tolyl | N-tolyl | b2 | H | b2 | H | a6 |
| 2-6-088 | N-tolyl | N-tolyl | b3 | H | b3 | H | a6 |
| 2-6-089 | N-Biph | N-Biph | H | H | H | H | H |
| 2-6-090 | N-Biph | N-Biph | t-bu | H | t-bu | H | H |
| 2-6-091 | N-Biph | N-Biph | methyl | H | methyl | H | H |
| 2-6-092 | N-Biph | N-Biph | t-bu | t-bu | t-bu | t-bu | t-bu |
| 2-6-093 | N-Biph | N-Biph | methyl | methyl | methyl | methyl | methyl |
| 2-6-094 | N-Biph | N-Biph | b1 | H | b1 | H | H |
| 2-6-095 | N-Biph | N-Biph | b2 | H | b2 | H | H |
| 2-6-096 | N-Biph | N-Biph | b3 | H | b3 | H | H |
| 2-6-097 | N-Biph | N-Biph | H | H | H | H | a1 |
| 2-6-098 | N-Biph | N-Biph | t-bu | H | t-bu | H | a1 |
| 2-6-099 | N-Biph | N-Biph | methyl | H | methyl | H | a1 |
| 2-6-100 | N-Biph | N-Biph | b1 | H | b1 | H | a1 |
| 2-6-101 | N-Biph | N-Biph | b2 | H | b2 | H | a1 |
| 2-6-102 | N-Biph | N-Biph | b3 | H | b3 | H | a1 |
| 2-6-103 | N-Biph | N-Biph | H | H | H | H | a2 |
| 2-6-104 | N-Biph | N-Biph | t-bu | H | t-bu | H | a2 |
| 2-6-105 | N-Biph | N-Biph | methyl | H | methyl | H | a2 |
| 2-6-106 | N-Biph | N-Biph | b1 | H | b1 | H | a2 |
| 2-6-107 | N-Biph | N-Biph | b2 | H | b2 | H | a2 |
| 2-6-108 | N-Biph | N-Biph | b3 | H | b3 | H | a2 |
| 2-6-109 | N-Biph | N-Biph | H | H | H | H | a3 |
| 2-6-110 | N-Biph | N-Biph | t-bu | H | t-bu | H | a3 |
| 2-6-111 | N-Biph | N-Biph | methyl | H | methyl | H | a3 |
| 2-6-112 | N-Biph | N-Biph | b1 | H | b1 | H | a3 |
| 2-6-113 | N-Biph | N-Biph | b2 | H | b2 | H | a3 |
| 2-6-114 | N-Biph | N-Biph | b3 | H | b3 | H | a3 |
| 2-6-115 | N-Biph | N-Biph | H | H | H | H | a4 |
| 2-6-116 | N-Biph | N-Biph | t-bu | H | t-bu | H | a4 |
| 2-6-117 | N-Biph | N-Biph | methyl | H | methyl | H | a4 |
| 2-6-118 | N-Biph | N-Biph | b1 | H | b1 | H | a4 |
| 2-6-119 | N-Biph | N-Biph | b2 | H | b2 | H | a4 |
| 2-6-120 | N-Biph | N-Biph | b3 | H | b3 | H | a4 |
| 2-6-121 | N-Biph | N-Biph | H | H | H | H | a5 |
| 2-6-122 | N-Biph | N-Biph | t-bu | H | t-bu | H | a5 |
| 2-6-123 | N-Biph | N-Biph | methyl | H | methyl | H | a5 |
| 2-6-124 | N-Biph | N-Biph | b1 | H | b1 | H | a5 |
| 2-6-125 | N-Biph | N-Biph | b2 | H | b2 | H | a5 |
| 2-6-126 | N-Biph | N-Biph | b3 | H | b3 | H | a5 |
| 2-6-127 | N-Biph | N-Biph | H | H | H | H | a6 |
| 2-6-128 | N-Biph | N-Biph | t-bu | H | t-bu | H | a6 |
| 2-6-129 | N-Biph | N-Biph | methyl | H | methyl | H | a6 |
| 2-6-130 | N-Biph | N-Biph | b1 | H | b1 | H | a6 |
| 2-6-131 | N-Biph | N-Biph | b2 | H | b2 | H | a6 |
| 2-6-132 | N-Biph | N-Biph | b3 | H | b3 | H | a6 |
| 2-6-133 | O | N—Ph | H | H | H | H | H |
| 2-6-134 | O | N—Ph | t-bu | H | t-bu | H | H |
| 2-6-135 | O | N—Ph | methyl | H | methyl | H | H |
| 2-6-136 | O | N—Ph | t-bu | t-bu | t-bu | t-bu | t-bu |
| 2-6-137 | O | N—Ph | methyl | methyl | methyl | methyl | methyl |
| 2-6-138 | O | N—Ph | b1 | H | b1 | H | H |
| 2-6-139 | O | N—Ph | b2 | H | b2 | H | H |
| 2-6-140 | O | N—Ph | b3 | H | b3 | H | H |
| 2-6-141 | O | N—Ph | H | H | H | H | a1 |
| 2-6-142 | O | N—Ph | t-bu | H | t-bu | H | a1 |
| 2-6-143 | O | N—Ph | methyl | H | methyl | H | a1 |
| 2-6-144 | O | N—Ph | b1 | H | b1 | H | a1 |
| 2-6-145 | O | N—Ph | b2 | H | b2 | H | a1 |
| 2-6-146 | O | N—Ph | b3 | H | b3 | H | a1 |
| 2-6-147 | O | N—Ph | H | H | H | H | a2 |
| 2-6-148 | O | N—Ph | t-bu | H | t-bu | H | a2 |
| 2-6-149 | O | N—Ph | methyl | H | methyl | H | a2 |
| 2-6-150 | O | N—Ph | b1 | H | b1 | H | a2 |
| 2-6-151 | O | N—Ph | b2 | H | b2 | H | a2 |
| 2-6-152 | O | N—Ph | b3 | H | b3 | H | a2 |
| 2-6-153 | O | N—Ph | H | H | H | H | a3 |
| 2-6-154 | O | N—Ph | t-bu | H | t-bu | H | a3 |
| 2-6-155 | O | N—Ph | methyl | H | methyl | H | a3 |
| 2-6-156 | O | N—Ph | b1 | H | b1 | H | a3 |
| 2-6-157 | O | N—Ph | b2 | H | b2 | H | a3 |
| 2-6-158 | O | N—Ph | b3 | H | b3 | H | a3 |
| 2-6-159 | O | N—Ph | H | H | H | H | a4 |
| 2-6-160 | O | N—Ph | t-bu | H | t-bu | H | a4 |
| 2-6-161 | O | N—Ph | methyl | H | methyl | H | a4 |
| 2-6-162 | O | N—Ph | b1 | H | b1 | H | a4 |
| 2-6-163 | O | N—Ph | b2 | H | b2 | H | a4 |
| 2-6-164 | O | N—Ph | b3 | H | b3 | H | a4 |
| 2-6-165 | O | N—Ph | H | H | H | H | a5 |
| 2-6-166 | O | N—Ph | t-bu | H | t-bu | H | a5 |
| 2-6-167 | O | N—Ph | methyl | H | methyl | H | a5 |
| 2-6-168 | O | N—Ph | b1 | H | b1 | H | a5 |
| 2-6-169 | O | N—Ph | b2 | H | b2 | H | a5 |
| 2-6-170 | O | N—Ph | b3 | H | b3 | H | a5 |
| 2-6-171 | O | N—Ph | H | H | H | H | a6 |
| 2-6-172 | O | N—Ph | t-bu | H | t-bu | H | a6 |
| 2-6-173 | O | N—Ph | methyl | H | methyl | H | a6 |
| 2-6-174 | O | N—Ph | b1 | H | b1 | H | a6 |
| 2-6-175 | O | N—Ph | b2 | H | b2 | H | a6 |
| 2-6-176 | O | N—Ph | b3 | H | b3 | H | a6 |
| 2-6-177 | S | N—Ph | H | H | H | H | H |
| 2-6-178 | S | N—Ph | t-bu | H | t-bu | H | H |
| 2-6-179 | S | N—Ph | methyl | H | methyl | H | H |
| 2-6-180 | S | N—Ph | t-bu | t-bu | t-bu | t-bu | t-bu |
| 2-6-181 | S | N—Ph | methyl | methyl | methyl | methyl | methyl |
| 2-6-182 | S | N—Ph | b1 | H | b1 | H | H |
| 2-6-183 | S | N—Ph | b2 | H | b2 | H | H |
| 2-6-184 | S | N—Ph | b3 | H | b3 | H | H |
| 2-6-185 | S | N—Ph | H | H | H | H | a1 |
| 2-6-186 | S | N—Ph | t-bu | H | t-bu | H | a1 |
| 2-6-187 | S | N—Ph | methyl | H | methyl | H | a1 |
| 2-6-188 | S | N—Ph | b1 | H | b1 | H | a1 |
| 2-6-189 | S | N—Ph | b2 | H | b2 | H | a1 |
| 2-6-190 | S | N—Ph | b3 | H | b3 | H | a1 |
| 2-6-191 | S | N—Ph | H | H | H | H | a2 |
| 2-6-192 | S | N—Ph | t-bu | H | t-bu | H | a2 |
| 2-6-193 | S | N—Ph | methyl | H | methyl | H | a2 |
| 2-6-194 | S | N—Ph | b1 | H | b1 | H | a2 |
| 2-6-195 | S | N—Ph | b2 | H | b2 | H | a2 |
| 2-6-196 | S | N—Ph | b3 | H | b3 | H | a2 |
| 2-6-197 | S | N—Ph | H | H | H | H | a3 |
| 2-6-198 | S | N—Ph | t-bu | H | t-bu | H | a3 |
| 2-6-199 | S | N—Ph | methyl | H | methyl | H | a3 |
| 2-6-200 | S | N—Ph | b1 | H | b1 | H | a3 |
| 2-6-201 | S | N—Ph | b2 | H | b2 | H | a3 |
| 2-6-202 | S | N—Ph | b3 | H | b3 | H | a3 |
| 2-6-203 | S | N—Ph | H | H | H | H | a4 |
| 2-6-204 | S | N—Ph | t-bu | H | t-bu | H | a4 |
| 2-6-205 | S | N—Ph | methyl | H | methyl | H | a4 |
| 2-6-206 | S | N—Ph | b1 | H | b1 | H | a4 |
| 2-6-207 | S | N—Ph | b2 | H | b2 | H | a4 |
| 2-6-208 | S | N—Ph | b3 | H | b3 | H | a4 |
| 2-6-209 | S | N—Ph | H | H | H | H | a5 |
| 2-6-210 | S | N—Ph | t-bu | H | t-bu | H | a5 |
| 2-6-211 | S | N—Ph | methyl | H | methyl | H | a5 |
| 2-6-212 | S | N—Ph | b1 | H | b1 | H | a5 |
| 2-6-213 | S | N—Ph | b2 | H | b2 | H | a5 |
| 2-6-214 | S | N—Ph | b3 | H | b3 | H | a5 |
| 2-6-215 | S | N—Ph | H | H | H | H | a6 |
| 2-6-216 | S | N—Ph | t-bu | H | t-bu | H | a6 |
| 2-6-217 | S | N—Ph | methyl | H | methyl | H | a6 |
| 2-6-218 | S | N—Ph | b1 | H | b1 | H | a6 |
| 2-6-219 | S | N—Ph | b2 | H | b2 | H | a6 |
| 2-6-220 | S | N—Ph | b3 | H | b3 | H | a6 |

Group 2-7
The compounds are represented by the formula 2-7 below, wherein CY₃ is a benzene group, X₁ is B, and Y1, Y2, R1b, R1d, R2a, R2c, (R3)a3 are each shown in the table 7 below, 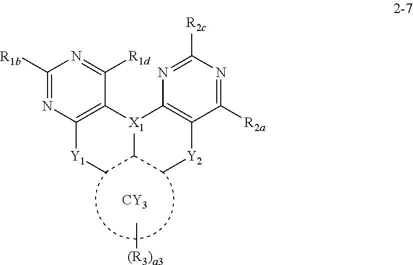
TABLE 7

| Compound | | | | | | | |
|---|---|---|---|---|---|---|---|
| No. | Y1 | Y2 | R1b | R1d | R2a | R2c | (R3)a3 |
| 2-7-001 | N—Ph | N—Ph | H | H | H | H | H |
| 2-7-002 | N—Ph | N—Ph | t-bu | H | t-bu | H | H |
| 2-7-003 | N—Ph | N—Ph | methyl | H | methyl | H | H |
| 2-7-004 | N—Ph | N—Ph | t-bu | t-bu | t-bu | t-bu | t-bu |
| 2-7-005 | N—Ph | N—Ph | methyl | methyl | methyl | methyl | methyl |
| 2-7-006 | N—Ph | N—Ph | b1 | H | b1 | H | H |
| 2-7-007 | N—Ph | N—Ph | b2 | H | b2 | H | H |
| 2-7-008 | N—Ph | N—Ph | b3 | H | b3 | H | H |
| 2-7-009 | N—Ph | N—Ph | H | H | H | H | a1 |
| 2-7-010 | N—Ph | N—Ph | t-bu | H | t-bu | t-bu | a1 |
| 2-7-011 | N—Ph | N—Ph | methyl | H | methyl | methyl | a1 |
| 2-7-012 | N—Ph | N—Ph | b1 | H | b1 | b1 | a1 |
| 2-7-013 | N—Ph | N—Ph | b2 | H | b2 | b2 | a1 |
| 2-7-014 | N—Ph | N—Ph | b3 | H | b3 | b3 | a1 |
| 2-7-015 | N—Ph | N—Ph | H | H | H | H | a2 |
| 2-7-016 | N—Ph | N—Ph | t-bu | H | t-bu | t-bu | a2 |
| 2-7-017 | N—Ph | N—Ph | methyl | H | methyl | methyl | a2 |
| 2-7-018 | N—Ph | N—Ph | b1 | H | b1 | b1 | a2 |
| 2-7-019 | N—Ph | N—Ph | b2 | H | b2 | b2 | a2 |
| 2-7-020 | N—Ph | N—Ph | b3 | H | b3 | b3 | a2 |
| 2-7-021 | N—Ph | N—Ph | H | H | H | H | a3 |
| 2-7-022 | N—Ph | N—Ph | t-bu | H | t-bu | t-bu | a3 |
| 2-7-023 | N—Ph | N—Ph | methyl | H | methyl | methyl | a3 |
| 2-7-024 | N—Ph | N—Ph | b1 | H | b1 | b1 | a3 |
| 2-7-025 | N—Ph | N—Ph | b2 | H | b2 | b2 | a3 |
| 2-7-026 | N—Ph | N—Ph | b3 | H | b3 | b3 | a3 |
| 2-7-027 | N—Ph | N—Ph | H | H | H | H | a4 |
| 2-7-028 | N—Ph | N—Ph | t-bu | H | t-bu | t-bu | a4 |
| 2-7-029 | N—Ph | N—Ph | methyl | H | methyl | methyl | a4 |
| 2-7-030 | N—Ph | N—Ph | b1 | H | b1 | b1 | a4 |
| 2-7-031 | N—Ph | N—Ph | b2 | H | b2 | b2 | a4 |
| 2-7-032 | N—Ph | N—Ph | b3 | H | b3 | b3 | a4 |
| 2-7-033 | N—Ph | N—Ph | H | H | H | H | a5 |
| 2-7-034 | N—Ph | N—Ph | t-bu | H | t-bu | t-bu | a5 |
| 2-7-035 | N—Ph | N—Ph | methyl | H | methyl | methyl | a5 |
| 2-7-036 | N—Ph | N—Ph | b1 | H | b1 | b1 | a5 |
| 2-7-037 | N—Ph | N—Ph | b2 | H | b2 | b2 | a5 |
| 2-7-038 | N—Ph | N—Ph | b3 | H | b3 | b3 | a5 |
| 2-7-039 | N—Ph | N—Ph | H | H | H | H | a6 |
| 2-7-040 | N—Ph | N—Ph | t-bu | H | t-bu | t-bu | a6 |
| 2-7-041 | N—Ph | N—Ph | methyl | H | methyl | methyl | a6 |
| 2-7-042 | N—Ph | N—Ph | b1 | H | b1 | b1 | a6 |
| 2-7-043 | N—Ph | N—Ph | b2 | H | b2 | b2 | a6 |
| 2-7-044 | N—Ph | N—Ph | b3 | H | b3 | b3 | a6 |
| 2-7-045 | N-tolyl | N-tolyl | H | H | H | H | H |
| 2-7-046 | N-tolyl | N-tolyl | t-bu | H | t-bu | H | H |
| 2-7-047 | N-tolyl | N-tolyl | methyl | H | methyl | H | H |
| 2-7-048 | N-tolyl | N-tolyl | t-bu | t-bu | t-bu | t-bu | t-bu |
| 2-7-049 | N-tolyl | N-tolyl | methyl | methyl | methyl | methyl | methyl |
| 2-7-050 | N-tolyl | N-tolyl | b1 | H | b1 | H | H |
| 2-7-051 | N-tolyl | N-tolyl | b2 | H | b2 | H | H |
| 2-7-052 | N-tolyl | N-tolyl | b3 | H | b3 | H | H |
| 2-7-053 | N-tolyl | N-tolyl | H | H | H | H | a1 |
| 2-7-054 | N-tolyl | N-tolyl | t-bu | H | t-bu | t-bu | a1 |
| 2-7-055 | N-tolyl | N-tolyl | methyl | H | methyl | methyl | a1 |
| 2-7-056 | N-tolyl | N-tolyl | b1 | H | b1 | b1 | a1 |
| 2-7-057 | N-tolyl | N-tolyl | b2 | H | b2 | b2 | a1 |
| 2-7-058 | N-tolyl | N-tolyl | b3 | H | b3 | b3 | a1 |
| 2-7-059 | N-tolyl | N-tolyl | H | H | H | H | a2 |
| 2-7-060 | N-tolyl | N-tolyl | t-bu | H | t-bu | t-bu | a2 |
| 2-7-061 | N-tolyl | N-tolyl | methyl | H | methyl | methyl | a2 |
| 2-7-062 | N-tolyl | N-tolyl | b1 | H | b1 | b1 | a2 |
| 2-7-063 | N-tolyl | N-tolyl | b2 | H | b2 | b2 | a2 |
| 2-7-064 | N-tolyl | N-tolyl | b3 | H | b3 | b3 | a2 |
| 2-7-065 | N-tolyl | N-tolyl | H | H | H | H | a3 |
| 2-7-066 | N-tolyl | N-tolyl | t-bu | H | t-bu | t-bu | a3 |
| 2-7-067 | N-tolyl | N-tolyl | methyl | H | methyl | methyl | a3 |
| 2-7-068 | N-tolyl | N-tolyl | b1 | H | b1 | b1 | a3 |
| 2-7-069 | N-tolyl | N-tolyl | b2 | H | b2 | b2 | a3 |
| 2-7-070 | N-tolyl | N-tolyl | b3 | H | b3 | b3 | a3 |
| 2-7-071 | N-tolyl | N-tolyl | H | H | H | H | a4 |
| 2-7-072 | N-tolyl | N-tolyl | t-bu | H | t-bu | t-bu | a4 |
| 2-7-073 | N-tolyl | N-tolyl | methyl | H | methyl | methyl | a4 |
| 2-7-074 | N-tolyl | N-tolyl | b1 | H | b1 | b1 | a4 |
| 2-7-075 | N-tolyl | N-tolyl | b2 | H | b2 | b2 | a4 |
| 2-7-076 | N-tolyl | N-tolyl | b3 | H | b3 | b3 | a4 |
| 2-7-077 | N-tolyl | N-tolyl | H | H | H | H | a5 |
| 2-7-078 | N-tolyl | N-tolyl | t-bu | H | t-bu | t-bu | a5 |
| 2-7-079 | N-tolyl | N-tolyl | methyl | H | methyl | methyl | a5 |
| 2-7-080 | N-tolyl | N-tolyl | b1 | H | b1 | b1 | a5 |
| 2-7-081 | N-tolyl | N-tolyl | b2 | H | b2 | b2 | a5 |
| 2-7-082 | N-tolyl | N-tolyl | b3 | H | b3 | b3 | a5 |
| 2-7-083 | N-tolyl | N-tolyl | H | H | H | H | a6 |
| 2-7-084 | N-tolyl | N-tolyl | t-bu | H | t-bu | t-bu | a6 |
| 2-7-085 | N-tolyl | N-tolyl | methyl | H | methyl | methyl | a6 |
| 2-7-086 | N-tolyl | N-tolyl | b1 | H | b1 | b1 | a6 |
| 2-7-087 | N-tolyl | N-tolyl | b2 | H | b2 | b2 | a6 |
| 2-7-088 | N-tolyl | N-tolyl | b3 | H | b3 | b3 | a6 |
| 2-7-089 | N-Biph | N-Biph | H | H | H | H | H |
| 2-7-090 | N-Biph | N-Biph | t-bu | H | t-bu | H | H |
| 2-7-091 | N-Biph | N-Biph | methyl | H | methyl | H | H |
| 2-7-092 | N-Biph | N-Biph | t-bu | t-bu | t-bu | t-bu | t-bu |
| 2-7-093 | N-Biph | N-Biph | methyl | methyl | methyl | methyl | methyl |
| 2-7-094 | N-Biph | N-Biph | b1 | H | b1 | H | H |
| 2-7-095 | N-Biph | N-Biph | b2 | H | b2 | H | H |
| 2-7-096 | N-Biph | N-Biph | b3 | H | b3 | H | H |
| 2-7-097 | N-Biph | N-Biph | H | H | H | H | a1 |
| 2-7-098 | N-Biph | N-Biph | t-bu | H | t-bu | t-bu | a1 |
| 2-7-099 | N-Biph | N-Biph | methyl | H | methyl | methyl | a1 |
| 2-7-100 | N-Biph | N-Biph | b1 | H | b1 | b1 | a1 |
| 2-7-101 | N-Biph | N-Biph | b2 | H | b2 | b2 | a1 |
| 2-7-102 | N-Biph | N-Biph | b3 | H | b3 | b3 | a1 |
| 2-7-103 | N-Biph | N-Biph | H | H | H | H | a2 |
| 2-7-104 | N-Biph | N-Biph | t-bu | H | t-bu | t-bu | a2 |
| 2-7-105 | N-Biph | N-Biph | methyl | H | methyl | methyl | a2 |
| 2-7-106 | N-Biph | N-Biph | b1 | H | b1 | b1 | a2 |
| 2-7-107 | N-Biph | N-Biph | b2 | H | b2 | b2 | a2 |
| 2-7-108 | N-Biph | N-Biph | b3 | H | b3 | b3 | a2 |
| 2-7-109 | N-Biph | N-Biph | H | H | H | H | a3 |
| 2-7-110 | N-Biph | N-Biph | t-bu | H | t-bu | t-bu | a3 |
| 2-7-111 | N-Biph | N-Biph | methyl | H | methyl | methyl | a3 |
| 2-7-112 | N-Biph | N-Biph | b1 | H | b1 | b1 | a3 |
| 2-7-113 | N-Biph | N-Biph | b2 | H | b2 | b2 | a3 |
| 2-7-114 | N-Biph | N-Biph | b3 | H | b3 | b3 | a3 |
| 2-7-115 | N-Biph | N-Biph | H | H | H | H | a4 |
| 2-7-116 | N-Biph | N-Biph | t-bu | H | t-bu | t-bu | a4 |
| 2-7-117 | N-Biph | N-Biph | methyl | H | methyl | methyl | a4 |
| 2-7-118 | N-Biph | N-Biph | b1 | H | b1 | b1 | a4 |
| 2-7-119 | N-Biph | N-Biph | b2 | H | b2 | b2 | a4 |
| 2-7-120 | N-Biph | N-Biph | b3 | H | b3 | b3 | a4 |
| 2-7-121 | N-Biph | N-Biph | H | H | H | H | a5 |
| 2-7-122 | N-Biph | N-Biph | t-bu | H | t-bu | t-bu | a5 |
| 2-7-123 | N-Biph | N-Biph | methyl | H | methyl | methyl | a5 |
| 2-7-124 | N-Biph | N-Biph | b1 | H | b1 | b1 | a5 |
| 2-7-125 | N-Biph | N-Biph | b2 | H | b2 | b2 | a5 |
| 2-7-126 | N-Biph | N-Biph | b3 | H | b3 | b3 | a5 |
| 2-7-127 | N-Biph | N-Biph | H | H | H | H | a6 |
| 2-7-128 | N-Biph | N-Biph | t-bu | H | t-bu | t-bu | a6 |
| 2-7-129 | N-Biph | N-Biph | methyl | H | methyl | methyl | a6 |
| 2-7-130 | N-Biph | N-Biph | b1 | H | b1 | b1 | a6 |
| 2-7-131 | N-Biph | N-Biph | b2 | H | b2 | b2 | a6 |
| 2-7-132 | N-Biph | N-Biph | b3 | H | b3 | b3 | a6 |
| 2-7-133 | O | N—Ph | H | H | H | H | H |
| 2-7-134 | O | N—Ph | t-bu | H | t-bu | H | H |
| 2-7-135 | O | N—Ph | methyl | H | methyl | H | H |
| 2-7-136 | O | N—Ph | t-bu | t-bu | t-bu | t-bu | t-bu |
| 2-7-137 | O | N—Ph | methyl | methyl | methyl | methyl | methyl |
| 2-7-138 | O | N—Ph | b1 | H | b1 | H | H |
| 2-7-139 | O | N—Ph | b2 | H | b2 | H | H |
| 2-7-140 | O | N—Ph | b3 | H | b3 | H | H |
| 2-7-141 | O | N—Ph | H | H | H | H | a1 |
| 2-7-142 | O | N—Ph | t-bu | H | t-bu | t-bu | a1 |
| 2-7-143 | O | N—Ph | methyl | H | methyl | methyl | a1 |
| 2-7-144 | O | N—Ph | b1 | H | b1 | b1 | a1 |
| 2-7-145 | O | N—Ph | b2 | H | b2 | b2 | a1 |
| 2-7-146 | O | N—Ph | b3 | H | b3 | b3 | a1 |
| 2-7-147 | O | N—Ph | H | H | H | H | a2 |
| 2-7-148 | O | N—Ph | t-bu | H | t-bu | t-bu | a2 |
| 2-7-149 | O | N—Ph | methyl | H | methyl | methyl | a2 |
| 2-7-150 | O | N—Ph | b1 | H | b1 | b1 | a2 |
| 2-7-151 | O | N—Ph | b2 | H | b2 | b2 | a2 |
| 2-7-152 | O | N—Ph | b3 | H | b3 | b3 | a2 |
| 2-7-153 | O | N—Ph | H | H | H | H | a3 |
| 2-7-154 | O | N—Ph | t-bu | H | t-bu | t-bu | a3 |
| 2-7-155 | O | N—Ph | methyl | H | methyl | methyl | a3 |
| 2-7-156 | O | N—Ph | b1 | H | b1 | b1 | a3 |
| 2-7-157 | O | N—Ph | b2 | H | b2 | b2 | a3 |
| 2-7-158 | O | N—Ph | b3 | H | b3 | b3 | a3 |
| 2-7-159 | O | N—Ph | H | H | H | H | a4 |
| 2-7-160 | O | N—Ph | t-bu | H | t-bu | t-bu | a4 |
| 2-7-161 | O | N—Ph | methyl | H | methyl | methyl | a4 |
| 2-7-162 | O | N—Ph | b1 | H | b1 | b1 | a4 |
| 2-7-163 | O | N—Ph | b2 | H | b2 | b2 | a4 |
| 2-7-164 | O | N—Ph | b3 | H | b3 | b3 | a4 |
| 2-7-165 | O | N—Ph | H | H | H | H | a5 |
| 2-7-166 | O | N—Ph | t-bu | H | t-bu | t-bu | a5 |
| 2-7-167 | O | N—Ph | methyl | H | methyl | methyl | a5 |
| 2-7-168 | O | N—Ph | b1 | H | b1 | b1 | a5 |
| 2-7-169 | O | N—Ph | b2 | H | b2 | b2 | a5 |
| 2-7-170 | O | N—Ph | b3 | H | b3 | b3 | a5 |
| 2-7-171 | O | N—Ph | H | H | H | H | a6 |
| 2-7-172 | O | N—Ph | t-bu | H | t-bu | t-bu | a6 |
| 2-7-173 | O | N—Ph | methyl | H | methyl | methyl | a6 |
| 2-7-174 | O | N—Ph | b1 | H | b1 | b1 | a6 |
| 2-7-175 | O | N—Ph | b2 | H | b2 | b2 | a6 |
| 2-7-176 | O | N—Ph | b3 | H | b3 | b3 | a6 |
| 2-7-177 | S | N—Ph | H | H | H | H | H |
| 2-7-178 | S | N—Ph | t-bu | H | t-bu | H | H |
| 2-7-179 | S | N—Ph | methyl | H | methyl | H | H |
| 2-7-180 | S | N—Ph | t-bu | t-bu | t-bu | t-bu | t-bu |
| 2-7-181 | S | N—Ph | methyl | methyl | methyl | methyl | methyl |
| 2-7-182 | S | N—Ph | b1 | H | b1 | H | H |
| 2-7-183 | S | N—Ph | b2 | H | b2 | H | H |
| 2-7-184 | S | N—Ph | b3 | H | b3 | H | H |
| 2-7-185 | S | N—Ph | H | H | H | H | a1 |
| 2-7-186 | S | N—Ph | t-bu | H | t-bu | t-bu | a1 |
| 2-7-187 | S | N—Ph | methyl | H | methyl | methyl | a1 |
| 2-7-188 | S | N—Ph | b1 | H | b1 | b1 | a1 |
| 2-7-189 | S | N—Ph | b2 | H | b2 | b2 | a1 |
| 2-7-190 | S | N—Ph | b3 | H | b3 | b3 | a1 |
| 2-7-191 | S | N—Ph | H | H | H | H | a2 |
| 2-7-192 | S | N—Ph | t-bu | H | t-bu | t-bu | a2 |
| 2-7-193 | S | N—Ph | methyl | H | methyl | methyl | a2 |
| 2-7-194 | S | N—Ph | b1 | H | b1 | b1 | a2 |
| 2-7-195 | S | N—Ph | b2 | H | b2 | b2 | a2 |
| 2-7-196 | S | N—Ph | b3 | H | b3 | b3 | a2 |
| 2-7-197 | S | N—Ph | H | H | H | H | a3 |
| 2-7-198 | S | N—Ph | t-bu | H | t-bu | t-bu | a3 |
| 2-7-199 | S | N—Ph | methyl | H | methyl | methyl | a3 |
| 2-7-200 | S | N—Ph | b1 | H | b1 | b1 | a3 |
| 2-7-201 | S | N—Ph | b2 | H | b2 | b2 | a3 |
| 2-7-202 | S | N—Ph | b3 | H | b3 | b3 | a3 |
| 2-7-203 | S | N—Ph | H | H | H | H | a4 |
| 2-7-204 | S | N—Ph | t-bu | H | t-bu | t-bu | a4 |
| 2-7-205 | S | N—Ph | methyl | H | methyl | methyl | a4 |
| 2-7-206 | S | N—Ph | b1 | H | b1 | b1 | a4 |
| 2-7-207 | S | N—Ph | b2 | H | b2 | b2 | a4 |
| 2-7-208 | S | N—Ph | b3 | H | b3 | b3 | a4 |
| 2-7-209 | S | N—Ph | H | H | H | H | a5 |
| 2-7-210 | S | N—Ph | t-bu | H | t-bu | t-bu | a5 |
| 2-7-211 | S | N—Ph | methyl | H | methyl | methyl | a5 |
| 2-7-212 | S | N—Ph | b1 | H | b1 | b1 | a5 |
| 2-7-213 | S | N—Ph | b2 | H | b2 | b2 | a5 |
| 2-7-214 | S | N—Ph | b3 | H | b3 | b3 | a5 |
| 2-7-215 | S | N—Ph | H | H | H | H | a6 |
| 2-7-216 | S | N—Ph | t-bu | H | t-bu | t-bu | a6 |
| 2-7-217 | S | N—Ph | methyl | H | methyl | methyl | a6 |
| 2-7-218 | S | N—Ph | b1 | H | b1 | b1 | a6 |
| 2-7-219 | S | N—Ph | b2 | H | b2 | b2 | a6 |
| 2-7-220 | S | N—Ph | b3 | H | b3 | b3 | a6 |

Group 2-8
The compounds are represented by the formula 2-8 below, wherein CY₃ is a benzene group, X₁ is B, and Y1, Y2, R1a, R1c, R2b, R2d, (R3)a3 are each shown in the table 8 below, 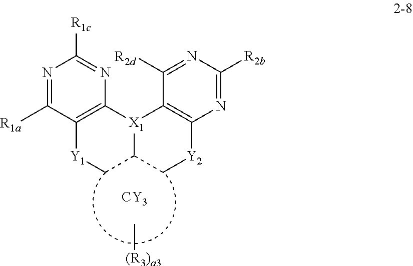
TABLE 8

| Compound | | | | | | | |
|---|---|---|---|---|---|---|---|
| No. | Y1 | Y2 | R1a | R1c | R2b | R2d | (R3)a3 |
| 2-8-001 | N—Ph | N—Ph | H | H | H | H | H |
| 2-8-002 | N—Ph | N—Ph | t-bu | H | t-bu | H | H |
| 2-8-003 | N—Ph | N—Ph | methyl | H | methyl | H | H |
| 2-8-004 | N—Ph | N—Ph | t-bu | t-bu | t-bu | t-bu | t-bu |
| 2-8-005 | N—Ph | N—Ph | methyl | methyl | methyl | methyl | methyl |
| 2-8-006 | N—Ph | N—Ph | b1 | H | b1 | H | H |
| 2-8-007 | N—Ph | N—Ph | b2 | H | b2 | H | H |
| 2-8-008 | N—Ph | N—Ph | b3 | H | b3 | H | H |
| 2-8-009 | N—Ph | N—Ph | H | H | H | H | a1 |
| 2-8-010 | N—Ph | N—Ph | t-bu | t-bu | t-bu | H | a1 |
| 2-8-011 | N—Ph | N—Ph | methyl | methyl | methyl | H | a1 |
| 2-8-012 | N—Ph | N—Ph | b1 | b1 | b1 | H | a1 |
| 2-8-013 | N—Ph | N—Ph | b2 | b2 | b2 | H | a1 |
| 2-8-014 | N—Ph | N—Ph | b3 | b3 | b3 | H | a1 |
| 2-8-015 | N—Ph | N—Ph | H | H | H | H | a2 |
| 2-8-016 | N—Ph | N—Ph | t-bu | t-bu | t-bu | H | a2 |
| 2-8-017 | N—Ph | N—Ph | methyl | methyl | methyl | H | a2 |
| 2-8-018 | N—Ph | N—Ph | b1 | b1 | b1 | H | a2 |
| 2-8-019 | N—Ph | N—Ph | b2 | b2 | b2 | H | a2 |
| 2-8-020 | N—Ph | N—Ph | b3 | b3 | b3 | H | a2 |
| 2-8-021 | N—Ph | N—Ph | H | H | H | H | a3 |
| 2-8-022 | N—Ph | N—Ph | t-bu | t-bu | t-bu | H | a3 |
| 2-8-023 | N—Ph | N—Ph | methyl | methyl | methyl | H | a3 |
| 2-8-024 | N—Ph | N—Ph | b1 | b1 | b1 | H | a3 |
| 2-8-025 | N—Ph | N—Ph | b2 | b2 | b2 | H | a3 |
| 2-8-026 | N—Ph | N—Ph | b3 | b3 | b3 | H | a3 |
| 2-8-027 | N—Ph | N—Ph | H | H | H | H | a4 |
| 2-8-028 | N—Ph | N—Ph | t-bu | t-bu | t-bu | H | a4 |
| 2-8-029 | N—Ph | N—Ph | methyl | methyl | methyl | H | a4 |
| 2-8-030 | N—Ph | N—Ph | b1 | b1 | b1 | H | a4 |
| 2-8-031 | N—Ph | N—Ph | b2 | b2 | b2 | H | a4 |
| 2-8-032 | N—Ph | N—Ph | b3 | b3 | b3 | H | a4 |
| 2-8-033 | N—Ph | N—Ph | H | H | H | H | a5 |
| 2-8-034 | N—Ph | N—Ph | t-bu | t-bu | t-bu | H | a5 |
| 2-8-035 | N—Ph | N—Ph | methyl | methyl | methyl | H | a5 |
| 2-8-036 | N—Ph | N—Ph | b1 | b1 | b1 | H | a5 |
| 2-8-037 | N—Ph | N—Ph | b2 | b2 | b2 | H | a5 |
| 2-8-038 | N—Ph | N—Ph | b3 | b3 | b3 | H | a5 |
| 2-8-039 | N—Ph | N—Ph | H | H | H | H | a6 |
| 2-8-040 | N—Ph | N—Ph | t-bu | t-bu | t-bu | H | a6 |
| 2-8-041 | N—Ph | N—Ph | methyl | methyl | methyl | H | a6 |
| 2-8-042 | N—Ph | N—Ph | b1 | b1 | b1 | H | a6 |
| 2-8-043 | N—Ph | N—Ph | b2 | b2 | b2 | H | a6 |
| 2-8-044 | N—Ph | N—Ph | b3 | b3 | b3 | H | a6 |
| 2-8-045 | N-tolyl | N-tolyl | H | H | H | H | H |
| 2-8-046 | N-tolyl | N-tolyl | t-bu | H | t-bu | H | H |
| 2-8-047 | N-tolyl | N-tolyl | methyl | H | methyl | H | H |
| 2-8-048 | N-tolyl | N-tolyl | t-bu | t-bu | t-bu | t-bu | t-bu |
| 2-8-049 | N-tolyl | N-tolyl | methyl | methyl | methyl | methyl | methyl |
| 2-8-050 | N-tolyl | N-tolyl | b1 | H | b1 | H | H |
| 2-8-051 | N-tolyl | N-tolyl | b2 | H | b2 | H | H |
| 2-8-052 | N-tolyl | N-tolyl | b3 | H | b3 | H | H |
| 2-8-053 | N-tolyl | N-tolyl | H | H | H | H | a1 |
| 2-8-054 | N-tolyl | N-tolyl | t-bu | t-bu | t-bu | H | a1 |
| 2-8-055 | N-tolyl | N-tolyl | methyl | methyl | methyl | H | a1 |
| 2-8-056 | N-tolyl | N-tolyl | b1 | b1 | b1 | H | a1 |
| 2-8-057 | N-tolyl | N-tolyl | b2 | b2 | b2 | H | a1 |
| 2-8-058 | N-tolyl | N-tolyl | b3 | b3 | b3 | H | a1 |
| 2-8-059 | N-tolyl | N-tolyl | H | H | H | H | a2 |
| 2-8-060 | N-tolyl | N-tolyl | t-bu | t-bu | t-bu | H | a2 |
| 2-8-061 | N-tolyl | N-tolyl | methyl | methyl | methyl | H | a2 |
| 2-8-062 | N-tolyl | N-tolyl | b1 | b1 | b1 | H | a2 |
| 2-8-063 | N-tolyl | N-tolyl | b2 | b2 | b2 | H | a2 |
| 2-8-064 | N-tolyl | N-tolyl | b3 | b3 | b3 | H | a2 |
| 2-8-065 | N-tolyl | N-tolyl | H | H | H | H | a3 |
| 2-8-066 | N-tolyl | N-tolyl | t-bu | t-bu | t-bu | H | a3 |
| 2-8-067 | N-tolyl | N-tolyl | methyl | methyl | methyl | H | a3 |
| 2-8-068 | N-tolyl | N-tolyl | b1 | b1 | b1 | H | a3 |
| 2-8-069 | N-tolyl | N-tolyl | b2 | b2 | b2 | H | a3 |
| 2-8-070 | N-tolyl | N-tolyl | b3 | b3 | b3 | H | a3 |
| 2-8-071 | N-tolyl | N-tolyl | H | H | H | H | a4 |
| 2-8-072 | N-tolyl | N-tolyl | t-bu | t-bu | t-bu | H | a4 |
| 2-8-073 | N-tolyl | N-tolyl | methyl | methyl | methyl | H | a4 |
| 2-8-074 | N-tolyl | N-tolyl | b1 | b1 | b1 | H | a4 |
| 2-8-075 | N-tolyl | N-tolyl | b2 | b2 | b2 | H | a4 |
| 2-8-076 | N-tolyl | N-tolyl | b3 | b3 | b3 | H | a4 |
| 2-8-077 | N-tolyl | N-tolyl | H | H | H | H | a5 |
| 2-8-078 | N-tolyl | N-tolyl | t-bu | t-bu | t-bu | H | a5 |
| 2-8-079 | N-tolyl | N-tolyl | methyl | methyl | methyl | H | a5 |
| 2-8-080 | N-tolyl | N-tolyl | b1 | b1 | b1 | H | a5 |
| 2-8-081 | N-tolyl | N-tolyl | b2 | b2 | b2 | H | a5 |
| 2-8-082 | N-tolyl | N-tolyl | b3 | b3 | b3 | H | a5 |
| 2-8-083 | N-tolyl | N-tolyl | H | H | H | H | a6 |
| 2-8-084 | N-tolyl | N-tolyl | t-bu | t-bu | t-bu | H | a6 |
| 2-8-085 | N-tolyl | N-tolyl | methyl | methyl | methyl | H | a6 |
| 2-8-086 | N-tolyl | N-tolyl | b1 | b1 | b1 | H | a6 |
| 2-8-087 | N-tolyl | N-tolyl | b2 | b2 | b2 | H | a6 |
| 2-8-088 | N-tolyl | N-tolyl | b3 | b3 | b3 | H | a6 |
| 2-8-089 | N-Biph | N-Biph | H | H | H | H | H |
| 2-8-090 | N-Biph | N-Biph | t-bu | H | t-bu | H | H |
| 2-8-091 | N-Biph | N-Biph | methyl | H | methyl | H | H |
| 2-8-092 | N-Biph | N-Biph | t-bu | t-bu | t-bu | t-bu | t-bu |
| 2-8-093 | N-Biph | N-Biph | methyl | methyl | methyl | methyl | methyl |
| 2-8-094 | N-Biph | N-Biph | b1 | H | b1 | H | H |
| 2-8-095 | N-Biph | N-Biph | b2 | H | b2 | H | H |
| 2-8-096 | N-Biph | N-Biph | b3 | H | b3 | H | H |
| 2-8-097 | N-Biph | N-Biph | H | H | H | H | a1 |
| 2-8-098 | N-Biph | N-Biph | t-bu | t-bu | t-bu | H | a1 |
| 2-8-099 | N-Biph | N-Biph | methyl | methyl | methyl | H | a1 |
| 2-8-100 | N-Biph | N-Biph | b1 | b1 | b1 | H | a1 |
| 2-8-101 | N-Biph | N-Biph | b2 | b2 | b2 | H | a1 |
| 2-8-102 | N-Biph | N-Biph | b3 | b3 | b3 | H | a1 |
| 2-8-103 | N-Biph | N-Biph | H | H | H | H | a2 |
| 2-8-104 | N-Biph | N-Biph | t-bu | t-bu | t-bu | H | a2 |
| 2-8-105 | N-Biph | N-Biph | methyl | methyl | methyl | H | a2 |
| 2-8-106 | N-Biph | N-Biph | b1 | b1 | b1 | H | a2 |
| 2-8-107 | N-Biph | N-Biph | b2 | b2 | b2 | H | a2 |
| 2-8-108 | N-Biph | N-Biph | b3 | b3 | b3 | H | a2 |
| 2-8-109 | N-Biph | N-Biph | H | H | H | H | a3 |
| 2-8-110 | N-Biph | N-Biph | t-bu | t-bu | t-bu | H | a3 |
| 2-8-111 | N-Biph | N-Biph | methyl | methyl | methyl | H | a3 |
| 2-8-112 | N-Biph | N-Biph | b1 | b1 | b1 | H | a3 |
| 2-8-113 | N-Biph | N-Biph | b2 | b2 | b2 | H | a3 |
| 2-8-114 | N-Biph | N-Biph | b3 | b3 | b3 | H | a3 |
| 2-8-115 | N-Biph | N-Biph | H | H | H | H | a4 |
| 2-8-116 | N-Biph | N-Biph | t-bu | t-bu | t-bu | H | a4 |
| 2-8-117 | N-Biph | N-Biph | methyl | methyl | methyl | H | a4 |
| 2-8-118 | N-Biph | N-Biph | b1 | b1 | b1 | H | a4 |
| 2-8-119 | N-Biph | N-Biph | b2 | b2 | b2 | H | a4 |
| 2-8-120 | N-Biph | N-Biph | b3 | b3 | b3 | H | a4 |
| 2-8-121 | N-Biph | N-Biph | H | H | H | H | a5 |
| 2-8-122 | N-Biph | N-Biph | t-bu | t-bu | t-bu | H | a5 |
| 2-8-123 | N-Biph | N-Biph | methyl | methyl | methyl | H | a5 |
| 2-8-124 | N-Biph | N-Biph | b1 | b1 | b1 | H | a5 |
| 2-8-125 | N-Biph | N-Biph | b2 | b2 | b2 | H | a5 |
| 2-8-126 | N-Biph | N-Biph | b3 | b3 | b3 | H | a5 |
| 2-8-127 | N-Biph | N-Biph | H | H | H | H | a6 |
| 2-8-128 | N-Biph | N-Biph | t-bu | t-bu | t-bu | H | a6 |
| 2-8-129 | N-Biph | N-Biph | methyl | methyl | methyl | H | a6 |
| 2-8-130 | N-Biph | N-Biph | b1 | b1 | b1 | H | a6 |
| 2-8-131 | N-Biph | N-Biph | b2 | b2 | b2 | H | a6 |
| 2-8-132 | N-Biph | N-Biph | b3 | b3 | b3 | H | a6 |
| 2-8-133 | O | N—Ph | H | H | H | H | H |
| 2-8-134 | O | N—Ph | t-bu | H | t-bu | H | H |
| 2-8-135 | O | N—Ph | methyl | H | methyl | H | H |
| 2-8-136 | O | N—Ph | t-bu | t-bu | t-bu | t-bu | t-bu |
| 2-8-137 | O | N—Ph | methyl | methyl | methyl | methyl | methyl |
| 2-8-138 | O | N—Ph | b1 | H | b1 | H | H |
| 2-8-139 | O | N—Ph | b2 | H | b2 | H | H |
| 2-8-140 | O | N—Ph | b3 | H | b3 | H | H |
| 2-8-141 | O | N—Ph | H | H | H | H | a1 |
| 2-8-142 | O | N—Ph | t-bu | t-bu | t-bu | H | a1 |
| 2-8-143 | O | N—Ph | methyl | methyl | methyl | H | a1 |
| 2-8-144 | O | N—Ph | b1 | b1 | b1 | H | a1 |
| 2-8-145 | O | N—Ph | b2 | b2 | b2 | H | a1 |
| 2-8-146 | O | N—Ph | b3 | b3 | b3 | H | a1 |
| 2-8-147 | O | N—Ph | H | H | H | H | a2 |
| 2-8-148 | O | N—Ph | t-bu | t-bu | t-bu | H | a2 |
| 2-8-149 | O | N—Ph | methyl | methyl | methyl | H | a2 |
| 2-8-150 | O | N—Ph | b1 | b1 | b1 | H | a2 |
| 2-8-151 | O | N—Ph | b2 | b2 | b2 | H | a2 |
| 2-8-152 | O | N—Ph | b3 | b3 | b3 | H | a2 |
| 2-8-153 | O | N—Ph | H | H | H | H | a3 |
| 2-8-154 | O | N—Ph | t-bu | t-bu | t-bu | H | a3 |
| 2-8-155 | O | N—Ph | methyl | methyl | methyl | H | a3 |
| 2-8-156 | O | N—Ph | b1 | b1 | b1 | H | a3 |
| 2-8-157 | O | N—Ph | b2 | b2 | b2 | H | a3 |
| 2-8-158 | O | N—Ph | b3 | b3 | b3 | H | a3 |
| 2-8-159 | O | N—Ph | H | H | H | H | a4 |
| 2-8-160 | O | N—Ph | t-bu | t-bu | t-bu | H | a4 |
| 2-8-161 | O | N—Ph | methyl | methyl | methyl | H | a4 |
| 2-8-162 | O | N—Ph | b1 | b1 | b1 | H | a4 |
| 2-8-163 | O | N—Ph | b2 | b2 | b2 | H | a4 |
| 2-8-164 | O | N—Ph | b3 | b3 | b3 | H | a4 |
| 2-8-165 | O | N—Ph | H | H | H | H | a5 |
| 2-8-166 | O | N—Ph | t-bu | t-bu | t-bu | H | a5 |
| 2-8-167 | O | N—Ph | methyl | methyl | methyl | H | a5 |
| 2-8-168 | O | N—Ph | b1 | b1 | b1 | H | a5 |
| 2-8-169 | O | N—Ph | b2 | b2 | b2 | H | a5 |
| 2-8-170 | O | N—Ph | b3 | b3 | b3 | H | a5 |
| 2-8-171 | O | N—Ph | H | H | H | H | a6 |
| 2-8-172 | O | N—Ph | t-bu | t-bu | t-bu | H | a6 |
| 2-8-173 | O | N—Ph | methyl | methyl | methyl | H | a6 |
| 2-8-174 | O | N—Ph | b1 | b1 | b1 | H | a6 |
| 2-8-175 | O | N—Ph | b2 | b2 | b2 | H | a6 |
| 2-8-176 | O | N—Ph | b3 | b3 | b3 | H | a6 |
| 2-8-177 | S | N—Ph | H | H | H | H | H |
| 2-8-178 | S | N—Ph | t-bu | H | t-bu | H | H |
| 2-8-179 | S | N—Ph | methyl | H | methyl | H | H |
| 2-8-180 | S | N—Ph | t-bu | t-bu | t-bu | t-bu | t-bu |
| 2-8-181 | S | N—Ph | methyl | methyl | methyl | methyl | methyl |
| 2-8-182 | S | N—Ph | b1 | H | b1 | H | H |
| 2-8-183 | S | N—Ph | b2 | H | b2 | H | H |
| 2-8-184 | S | N—Ph | b3 | H | b3 | H | H |
| 2-8-185 | S | N—Ph | H | H | H | H | a1 |
| 2-8-186 | S | N—Ph | t-bu | t-bu | t-bu | H | a1 |
| 2-8-187 | S | N—Ph | methyl | methyl | methyl | H | a1 |
| 2-8-188 | S | N—Ph | b1 | b1 | b1 | H | a1 |
| 2-8-189 | S | N—Ph | b2 | b2 | b2 | H | a1 |
| 2-8-190 | S | N—Ph | b3 | b3 | b3 | H | a1 |
| 2-8-191 | S | N—Ph | H | H | H | H | a2 |
| 2-8-192 | S | N—Ph | t-bu | t-bu | t-bu | H | a2 |
| 2-8-193 | S | N—Ph | methyl | methyl | methyl | H | a2 |
| 2-8-194 | S | N—Ph | b1 | b1 | b1 | H | a2 |
| 2-8-195 | S | N—Ph | b2 | b2 | b2 | H | a2 |
| 2-8-196 | S | N—Ph | b3 | b3 | b3 | H | a2 |
| 2-8-197 | S | N—Ph | H | H | H | H | a3 |
| 2-8-198 | S | N—Ph | t-bu | t-bu | t-bu | H | a3 |
| 2-8-199 | S | N—Ph | methyl | methyl | methyl | H | a3 |
| 2-8-200 | S | N—Ph | b1 | b1 | b1 | H | a3 |
| 2-8-201 | S | N—Ph | b2 | b2 | b2 | H | a3 |
| 2-8-202 | S | N—Ph | b3 | b3 | b3 | H | a3 |
| 2-8-203 | S | N—Ph | H | H | H | H | a4 |
| 2-8-204 | S | N—Ph | t-bu | t-bu | t-bu | H | a4 |
| 2-8-205 | S | N—Ph | methyl | methyl | methyl | H | a4 |
| 2-8-206 | S | N—Ph | b1 | b1 | b1 | H | a4 |
| 2-8-207 | S | N—Ph | b2 | b2 | b2 | H | a4 |
| 2-8-208 | S | N—Ph | b3 | b3 | b3 | H | a4 |
| 2-8-209 | S | N—Ph | H | H | H | H | a5 |
| 2-8-210 | S | N—Ph | t-bu | t-bu | t-bu | H | a5 |
| 2-8-211 | S | N—Ph | methyl | methyl | methyl | H | a5 |
| 2-8-212 | S | N—Ph | b1 | b1 | b1 | H | a5 |
| 2-8-213 | S | N—Ph | b2 | b2 | b2 | H | a5 |
| 2-8-214 | S | N—Ph | b3 | b3 | b3 | H | a5 |
| 2-8-215 | S | N—Ph | H | H | H | H | a6 |
| 2-8-216 | S | N—Ph | t-bu | t-bu | t-bu | H | a6 |
| 2-8-217 | S | N—Ph | methyl | methyl | methyl | H | a6 |
| 2-8-218 | S | N—Ph | b1 | b1 | b1 | H | a6 |
| 2-8-219 | S | N—Ph | b2 | b2 | b2 | H | a6 |
| 2-8-220 | S | N—Ph | b3 | b3 | b3 | H | a6 |

Group 2-9
The compounds are represented by the formula 2-9 below, wherein CY₃ is a benzene group, X₁ is B, and Y1, Y2, R1a, R1c, R2a, R2c, (R3)a3 are each shown in the table 9 below, 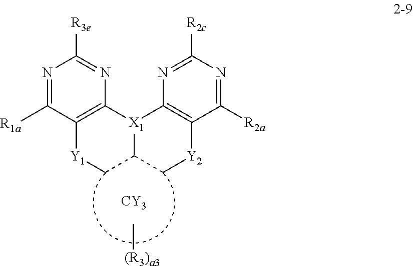
TABLE 9

| Compound | | | | | | | |
|---|---|---|---|---|---|---|---|
| No. | Y1 | Y2 | R1a | R1c | R2a | R2c | (R3)a3 |
| 2-9-001 | N—Ph | N—Ph | H | H | H | H | H |
| 2-9-002 | N—Ph | N—Ph | t-bu | H | t-bu | H | H |
| 2-9-003 | N—Ph | N—Ph | methyl | H | methyl | H | H |
| 2-9-004 | N—Ph | N—Ph | t-bu | t-bu | t-bu | t-bu | t-bu |
| 2-9-005 | N—Ph | N—Ph | methyl | methyl | methyl | methyl | methyl |
| 2-9-006 | N—Ph | N—Ph | b1 | H | b1 | H | H |
| 2-9-007 | N—Ph | N—Ph | b2 | H | b2 | H | H |
| 2-9-008 | N—Ph | N—Ph | b3 | H | b3 | H | H |
| 2-9-009 | N—Ph | N—Ph | H | H | H | H | a1 |
| 2-9-010 | N—Ph | N—Ph | t-bu | t-bu | t-bu | t-bu | a1 |
| 2-9-011 | N—Ph | N—Ph | methyl | methyl | methyl | methyl | a1 |
| 2-9-012 | N—Ph | N—Ph | b1 | b1 | b1 | b1 | a1 |
| 2-9-013 | N—Ph | N—Ph | b2 | b2 | b2 | b2 | a1 |
| 2-9-014 | N—Ph | N—Ph | b3 | b3 | b3 | b3 | a1 |
| 2-9-015 | N—Ph | N—Ph | H | H | H | H | a2 |
| 2-9-016 | N—Ph | N—Ph | t-bu | t-bu | t-bu | t-bu | a2 |
| 2-9-017 | N—Ph | N—Ph | methyl | methyl | methyl | methyl | a2 |
| 2-9-018 | N—Ph | N—Ph | b1 | b1 | b1 | b1 | a2 |
| 2-9-019 | N—Ph | N—Ph | b2 | b2 | b2 | b2 | a2 |
| 2-9-020 | N—Ph | N—Ph | b3 | b3 | b3 | b3 | a2 |
| 2-9-021 | N—Ph | N—Ph | H | H | H | H | a3 |
| 2-9-022 | N—Ph | N—Ph | t-bu | t-bu | t-bu | t-bu | a3 |
| 2-9-023 | N—Ph | N—Ph | methyl | methyl | methyl | methyl | a3 |
| 2-9-024 | N—Ph | N—Ph | b1 | b1 | b1 | b1 | a3 |
| 2-9-025 | N—Ph | N—Ph | b2 | b2 | b2 | b2 | a3 |
| 2-9-026 | N—Ph | N—Ph | b3 | b3 | b3 | b3 | a3 |
| 2-9-027 | N—Ph | N—Ph | H | H | H | H | a4 |
| 2-9-028 | N—Ph | N—Ph | t-bu | t-bu | t-bu | t-bu | a4 |
| 2-9-029 | N—Ph | N—Ph | methyl | methyl | methyl | methyl | a4 |
| 2-9-030 | N—Ph | N—Ph | b1 | b1 | b1 | b1 | a4 |
| 2-9-031 | N—Ph | N—Ph | b2 | b2 | b2 | b2 | a4 |
| 2-9-032 | N—Ph | N—Ph | b3 | b3 | b3 | b3 | a4 |
| 2-9-033 | N—Ph | N—Ph | H | H | H | H | a5 |
| 2-9-034 | N—Ph | N—Ph | t-bu | t-bu | t-bu | t-bu | a5 |
| 2-9-035 | N—Ph | N—Ph | methyl | methyl | methyl | methyl | a5 |
| 2-9-036 | N—Ph | N—Ph | b1 | b1 | b1 | b1 | a5 |
| 2-9-037 | N—Ph | N—Ph | b2 | b2 | b2 | b2 | a5 |
| 2-9-038 | N—Ph | N—Ph | b3 | b3 | b3 | b3 | a5 |
| 2-9-039 | N—Ph | N—Ph | H | H | H | H | a6 |
| 2-9-040 | N—Ph | N—Ph | t-bu | t-bu | t-bu | t-bu | a6 |
| 2-9-041 | N—Ph | N—Ph | methyl | methyl | methyl | methyl | a6 |
| 2-9-042 | N—Ph | N—Ph | b1 | b1 | b1 | b1 | a6 |
| 2-9-043 | N—Ph | N—Ph | b2 | b2 | b2 | b2 | a6 |
| 2-9-044 | N—Ph | N—Ph | b3 | b3 | b3 | b3 | a6 |
| 2-9-045 | N-tolyl | N-tolyl | H | H | H | H | H |
| 2-9-046 | N-tolyl | N-tolyl | t-bu | H | t-bu | H | H |
| 2-9-047 | N-tolyl | N-tolyl | methyl | H | methyl | H | H |
| 2-9-048 | N-tolyl | N-tolyl | t-bu | t-bu | t-bu | t-bu | t-bu |
| 2-9-049 | N-tolyl | N-tolyl | methyl | methyl | methyl | methyl | methyl |
| 2-9-050 | N-tolyl | N-tolyl | b1 | H | b1 | H | H |
| 2-9-051 | N-tolyl | N-tolyl | b2 | H | b2 | H | H |
| 2-9-052 | N-tolyl | N-tolyl | b3 | H | b3 | H | H |
| 2-9-053 | N-tolyl | N-tolyl | H | H | H | H | a1 |
| 2-9-054 | N-tolyl | N-tolyl | t-bu | t-bu | t-bu | t-bu | a1 |
| 2-9-055 | N-tolyl | N-tolyl | methyl | methyl | methyl | methyl | a1 |
| 2-9-056 | N-tolyl | N-tolyl | b1 | b1 | b1 | b1 | a1 |
| 2-9-057 | N-tolyl | N-tolyl | b2 | b2 | b2 | b2 | a1 |
| 2-9-058 | N-tolyl | N-tolyl | b3 | b3 | b3 | b3 | a1 |
| 2-9-059 | N-tolyl | N-tolyl | H | H | H | H | a2 |
| 2-9-060 | N-tolyl | N-tolyl | t-bu | t-bu | t-bu | t-bu | a2 |
| 2-9-061 | N-tolyl | N-tolyl | methyl | methyl | methyl | methyl | a2 |
| 2-9-062 | N-tolyl | N-tolyl | b1 | b1 | b1 | b1 | a2 |
| 2-9-063 | N-tolyl | N-tolyl | b2 | b2 | b2 | b2 | a2 |
| 2-9-064 | N-tolyl | N-tolyl | b3 | b3 | b3 | b3 | a2 |
| 2-9-065 | N-tolyl | N-tolyl | H | H | H | H | a3 |
| 2-9-066 | N-tolyl | N-tolyl | t-bu | t-bu | t-bu | t-bu | a3 |
| 2-9-067 | N-tolyl | N-tolyl | methyl | methyl | methyl | methyl | a3 |
| 2-9-068 | N-tolyl | N-tolyl | b1 | b1 | b1 | b1 | a3 |
| 2-9-069 | N-tolyl | N-tolyl | b2 | b2 | b2 | b2 | a3 |
| 2-9-070 | N-tolyl | N-tolyl | b3 | b3 | b3 | b3 | a3 |
| 2-9-071 | N-tolyl | N-tolyl | H | H | H | H | a4 |
| 2-9-072 | N-tolyl | N-tolyl | t-bu | t-bu | t-bu | t-bu | a4 |
| 2-9-073 | N-tolyl | N-tolyl | methyl | methyl | methyl | methyl | a4 |
| 2-9-074 | N-tolyl | N-tolyl | b1 | b1 | b1 | b1 | a4 |
| 2-9-075 | N-tolyl | N-tolyl | b2 | b2 | b2 | b2 | a4 |
| 2-9-076 | N-tolyl | N-tolyl | b3 | b3 | b3 | b3 | a4 |
| 2-9-077 | N-tolyl | N-tolyl | H | H | H | H | a5 |
| 2-9-078 | N-tolyl | N-tolyl | t-bu | t-bu | t-bu | t-bu | a5 |
| 2-9-079 | N-tolyl | N-tolyl | methyl | methyl | methyl | methyl | a5 |
| 2-9-080 | N-tolyl | N-tolyl | b1 | b1 | b1 | b1 | a5 |
| 2-9-081 | N-tolyl | N-tolyl | b2 | b2 | b2 | b2 | a5 |
| 2-9-082 | N-tolyl | N-tolyl | b3 | b3 | b3 | b3 | a5 |
| 2-9-083 | N-tolyl | N-tolyl | H | H | H | H | a6 |
| 2-9-084 | N-tolyl | N-tolyl | t-bu | t-bu | t-bu | t-bu | a6 |
| 2-9-085 | N-tolyl | N-tolyl | methyl | methyl | methyl | methyl | a6 |
| 2-9-086 | N-tolyl | N-tolyl | b1 | b1 | b1 | b1 | a6 |
| 2-9-087 | N-tolyl | N-tolyl | b2 | b2 | b2 | b2 | a6 |
| 2-9-088 | N-tolyl | N-tolyl | b3 | b3 | b3 | b3 | a6 |
| 2-9-089 | N-Biph | N-Biph | H | H | H | H | H |
| 2-9-090 | N-Biph | N-Biph | t-bu | H | t-bu | H | H |
| 2-9-091 | N-Biph | N-Biph | methyl | H | methyl | H | H |
| 2-9-092 | N-Biph | N-Biph | t-bu | t-bu | t-bu | t-bu | t-bu |
| 2-9-093 | N-Biph | N-Biph | methyl | methyl | methyl | methyl | methyl |
| 2-9-094 | N-Biph | N-Biph | b1 | H | b1 | H | H |
| 2-9-095 | N-Biph | N-Biph | b2 | H | b2 | H | H |
| 2-9-096 | N-Biph | N-Biph | b3 | H | b3 | H | H |
| 2-9-097 | N-Biph | N-Biph | H | H | H | H | a1 |
| 2-9-098 | N-Biph | N-Biph | t-bu | t-bu | t-bu | t-bu | a1 |
| 2-9-099 | N-Biph | N-Biph | methyl | methyl | methyl | methyl | a1 |
| 2-9-100 | N-Biph | N-Biph | b1 | b1 | b1 | b1 | a1 |
| 2-9-101 | N-Biph | N-Biph | b2 | b2 | b2 | b2 | a1 |
| 2-9-102 | N-Biph | N-Biph | b3 | b3 | b3 | b3 | a1 |
| 2-9-103 | N-Biph | N-Biph | H | H | H | H | a2 |
| 2-9-104 | N-Biph | N-Biph | t-bu | t-bu | t-bu | t-bu | a2 |
| 2-9-105 | N-Biph | N-Biph | methyl | methyl | methyl | methyl | a2 |
| 2-9-106 | N-Biph | N-Biph | b1 | b1 | b1 | b1 | a2 |
| 2-9-107 | N-Biph | N-Biph | b2 | b2 | b2 | b2 | a2 |
| 2-9-108 | N-Biph | N-Biph | b3 | b3 | b3 | b3 | a2 |
| 2-9-109 | N-Biph | N-Biph | H | H | H | H | a3 |
| 2-9-110 | N-Biph | N-Biph | t-bu | t-bu | t-bu | t-bu | a3 |
| 2-9-111 | N-Biph | N-Biph | methyl | methyl | methyl | methyl | a3 |
| 2-9-112 | N-Biph | N-Biph | b1 | b1 | b1 | b1 | a3 |
| 2-9-113 | N-Biph | N-Biph | b2 | b2 | b2 | b2 | a3 |
| 2-9-114 | N-Biph | N-Biph | b3 | b3 | b3 | b3 | a3 |
| 2-9-115 | N-Biph | N-Biph | H | H | H | H | a4 |
| 2-9-116 | N-Biph | N-Biph | t-bu | t-bu | t-bu | t-bu | a4 |
| 2-9-117 | N-Biph | N-Biph | methyl | methyl | methyl | methyl | a4 |
| 2-9-118 | N-Biph | N-Biph | b1 | b1 | b1 | b1 | a4 |
| 2-9-119 | N-Biph | N-Biph | b2 | b2 | b2 | b2 | a4 |
| 2-9-120 | N-Biph | N-Biph | b3 | b3 | b3 | b3 | a4 |
| 2-9-121 | N-Biph | N-Biph | H | H | H | H | a5 |
| 2-9-122 | N-Biph | N-Biph | t-bu | t-bu | t-bu | t-bu | a5 |
| 2-9-123 | N-Biph | N-Biph | methyl | methyl | methyl | methyl | a5 |
| 2-9-124 | N-Biph | N-Biph | b1 | b1 | b1 | b1 | a5 |
| 2-9-125 | N-Biph | N-Biph | b2 | b2 | b2 | b2 | a5 |
| 2-9-126 | N-Biph | N-Biph | b3 | b3 | b3 | b3 | a5 |
| 2-9-127 | N-Biph | N-Biph | H | H | H | H | a6 |
| 2-9-128 | N-Biph | N-Biph | t-bu | t-bu | t-bu | t-bu | a6 |
| 2-9-129 | N-Biph | N-Biph | methyl | methyl | methyl | methyl | a6 |
| 2-9-130 | N-Biph | N-Biph | b1 | b1 | b1 | b1 | a6 |
| 2-9-131 | N-Biph | N-Biph | b2 | b2 | b2 | b2 | a6 |
| 2-9-132 | N-Biph | N-Biph | b3 | b3 | b3 | b3 | a6 |
| 2-9-133 | O | N—Ph | H | H | H | H | H |
| 2-9-134 | O | N—Ph | t-bu | H | t-bu | H | H |
| 2-9-135 | O | N—Ph | methyl | H | methyl | H | H |
| 2-9-136 | O | N—Ph | t-bu | t-bu | t-bu | t-bu | t-bu |
| 2-9-137 | O | N—Ph | methyl | methyl | methyl | methyl | methyl |
| 2-9-138 | O | N—Ph | b1 | H | b1 | H | H |
| 2-9-139 | O | N—Ph | b2 | H | b2 | H | H |
| 2-9-140 | O | N—Ph | b3 | H | b3 | H | H |
| 2-9-141 | O | N—Ph | H | H | H | H | a1 |
| 2-9-142 | O | N—Ph | t-bu | t-bu | t-bu | t-bu | a1 |
| 2-9-143 | O | N—Ph | methyl | methyl | methyl | methyl | a1 |
| 2-9-144 | O | N—Ph | b1 | b1 | b1 | b1 | a1 |
| 2-9-145 | O | N—Ph | b2 | b2 | b2 | b2 | a1 |
| 2-9-146 | O | N—Ph | b3 | b3 | b3 | b3 | a1 |
| 2-9-147 | O | N—Ph | H | H | H | H | a2 |
| 2-9-148 | O | N—Ph | t-bu | t-bu | t-bu | t-bu | a2 |
| 2-9-149 | O | N—Ph | methyl | methyl | methyl | methyl | a2 |
| 2-9-150 | O | N—Ph | b1 | b1 | b1 | b1 | a2 |
| 2-9-151 | O | N—Ph | b2 | b2 | b2 | b2 | a2 |
| 2-9-152 | O | N—Ph | b3 | b3 | b3 | b3 | a2 |
| 2-9-153 | O | N—Ph | H | H | H | H | a3 |
| 2-9-154 | O | N—Ph | t-bu | t-bu | t-bu | t-bu | a3 |
| 2-9-155 | O | N—Ph | methyl | methyl | methyl | methyl | a3 |
| 2-9-156 | O | N—Ph | b1 | b1 | b1 | b1 | a3 |
| 2-9-157 | O | N—Ph | b2 | b2 | b2 | b2 | a3 |
| 2-9-158 | O | N—Ph | b3 | b3 | b3 | b3 | a3 |
| 2-9-159 | O | N—Ph | H | H | H | H | a4 |
| 2-9-160 | O | N—Ph | t-bu | t-bu | t-bu | t-bu | a4 |
| 2-9-161 | O | N—Ph | methyl | methyl | methyl | methyl | a4 |
| 2-9-162 | O | N—Ph | b1 | b1 | b1 | b1 | a4 |
| 2-9-163 | O | N—Ph | b2 | b2 | b2 | b2 | a4 |
| 2-9-164 | O | N—Ph | b3 | b3 | b3 | b3 | a4 |
| 2-9-165 | O | N—Ph | H | H | H | H | a5 |
| 2-9-166 | O | N—Ph | t-bu | t-bu | t-bu | t-bu | a5 |
| 2-9-167 | O | N—Ph | methyl | methyl | methyl | methyl | a5 |
| 2-9-168 | O | N—Ph | b1 | b1 | b1 | b1 | a5 |
| 2-9-169 | O | N—Ph | b2 | b2 | b2 | b2 | a5 |
| 2-9-170 | O | N—Ph | b3 | b3 | b3 | b3 | a5 |
| 2-9-171 | O | N—Ph | H | H | H | H | a6 |
| 2-9-172 | O | N—Ph | t-bu | t-bu | t-bu | t-bu | a6 |
| 2-9-173 | O | N—Ph | methyl | methyl | methyl | methyl | a6 |
| 2-9-174 | O | N—Ph | b1 | b1 | b1 | b1 | a6 |
| 2-9-175 | O | N—Ph | b2 | b2 | b2 | b2 | a6 |
| 2-9-176 | O | N—Ph | b3 | b3 | b3 | b3 | a6 |
| 2-9-177 | S | N—Ph | H | H | H | H | H |
| 2-9-178 | S | N—Ph | t-bu | H | t-bu | H | H |
| 2-9-179 | S | N—Ph | methyl | H | methyl | H | H |
| 2-9-180 | S | N—Ph | t-bu | t-bu | t-bu | t-bu | t-bu |
| 2-9-181 | S | N—Ph | methyl | methyl | methyl | methyl | methyl |
| 2-9-182 | S | N—Ph | b1 | H | b1 | H | H |
| 2-9-183 | S | N—Ph | b2 | H | b2 | H | H |
| 2-9-184 | S | N—Ph | b3 | H | b3 | H | H |
| 2-9-185 | S | N—Ph | H | H | H | H | a1 |
| 2-9-186 | S | N—Ph | t-bu | t-bu | t-bu | t-bu | a1 |
| 2-9-187 | S | N—Ph | methyl | methyl | methyl | methyl | a1 |
| 2-9-188 | S | N—Ph | b1 | b1 | b1 | b1 | a1 |
| 2-9-189 | S | N—Ph | b2 | b2 | b2 | b2 | a1 |
| 2-9-190 | S | N—Ph | b3 | b3 | b3 | b3 | a1 |
| 2-9-191 | S | N—Ph | H | H | H | H | a2 |
| 2-9-192 | S | N—Ph | t-bu | t-bu | t-bu | t-bu | a2 |
| 2-9-193 | S | N—Ph | methyl | methyl | methyl | methyl | a2 |
| 2-9-194 | S | N—Ph | b1 | b1 | b1 | b1 | a2 |
| 2-9-195 | S | N—Ph | b2 | b2 | b2 | b2 | a2 |
| 2-9-196 | S | N—Ph | b3 | b3 | b3 | b3 | a2 |
| 2-9-197 | S | N—Ph | H | H | H | H | a3 |
| 2-9-198 | S | N—Ph | t-bu | t-bu | t-bu | t-bu | a3 |
| 2-9-199 | S | N—Ph | methyl | methyl | methyl | methyl | a3 |
| 2-9-200 | S | N—Ph | b1 | b1 | b1 | b1 | a3 |
| 2-9-201 | S | N—Ph | b2 | b2 | b2 | b2 | a3 |
| 2-9-202 | S | N—Ph | b3 | b3 | b3 | b3 | a3 |
| 2-9-203 | S | N—Ph | H | H | H | H | a4 |
| 2-9-204 | S | N—Ph | t-bu | t-bu | t-bu | t-bu | a4 |
| 2-9-205 | S | N—Ph | methyl | methyl | methyl | methyl | a4 |
| 2-9-206 | S | N—Ph | b1 | b1 | b1 | b1 | a4 |
| 2-9-207 | S | N—Ph | b2 | b2 | b2 | b2 | a4 |
| 2-9-208 | S | N—Ph | b3 | b3 | b3 | b3 | a4 |
| 2-9-209 | S | N—Ph | H | H | H | H | a5 |
| 2-9-210 | S | N—Ph | t-bu | t-bu | t-bu | t-bu | a5 |
| 2-9-211 | S | N—Ph | methyl | methyl | methyl | methyl | a5 |
| 2-9-212 | S | N—Ph | b1 | b1 | b1 | b1 | a5 |
| 2-9-213 | S | N—Ph | b2 | b2 | b2 | b2 | a5 |
| 2-9-214 | S | N—Ph | b3 | b3 | b3 | b3 | a5 |
| 2-9-215 | S | N—Ph | H | H | H | H | a6 |
| 2-9-216 | S | N—Ph | t-bu | t-bu | t-bu | t-bu | a6 |
| 2-9-217 | S | N—Ph | methyl | methyl | methyl | methyl | a6 |
| 2-9-218 | S | N—Ph | b1 | b1 | b1 | b1 | a6 |
| 2-9-219 | S | N—Ph | b2 | b2 | b2 | b2 | a6 |
| 2-9-220 | S | N—Ph | b3 | b3 | b3 | b3 | a6 |

Group 2-10
The compounds are represented by the formula 2-10 below, wherein CY₂ is a benzene group, X₁ is B, and Y1, Y2, R1b, R1d, R3a, (R2)a2 are each shown in the table 10 below, 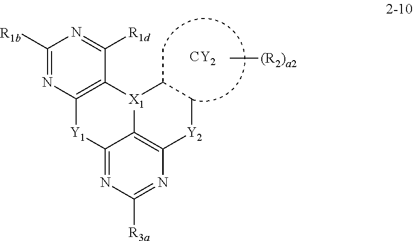
TABLE 10

| Compound | | | | | | |
|---|---|---|---|---|---|---|
| No. | Y1 | Y2 | R1b | R1d | R3a | (R2)a2 |
| 2-10-001 | N-Ph | N-Ph | H | H | H | H |
| 2-10-002 | N-Ph | N-Ph | t-bu | H | t-bu | H |
| 2-10-003 | N-Ph | N-Ph | methyl | H | methyl | H |
| 2-10-004 | N-Ph | N-Ph | t-bu | t-bu | t-bu | t-bu |
| 2-10-005 | N-Ph | N-Ph | methyl | methyl | methyl | methyl |
| 2-10-006 | N-Ph | N-Ph | b1 | H | b1 | H |
| 2-10-007 | N-Ph | N-Ph | b2 | H | b2 | H |
| 2-10-008 | N-Ph | N-Ph | b3 | H | b3 | H |
| 2-10-009 | N-Ph | N-Ph | H | H | H | a1 |
| 2-10-010 | N-Ph | N-Ph | t-bu | H | t-bu | a1 |
| 2-10-011 | N-Ph | N-Ph | methyl | H | methyl | a1 |
| 2-10-012 | N-Ph | N-Ph | b1 | H | b1 | a1 |
| 2-10-013 | N-Ph | N-Ph | b2 | H | b2 | a1 |
| 2-10-014 | N-Ph | N-Ph | b3 | H | b3 | a1 |
| 2-10-015 | N-Ph | N-Ph | H | H | H | a2 |
| 2-10-016 | N-Ph | N-Ph | t-bu | H | t-bu | a2 |
| 2-10-017 | N-Ph | N-Ph | methyl | H | methyl | a2 |
| 2-10-018 | N-Ph | N-Ph | b1 | H | b1 | a2 |
| 2-10-019 | N-Ph | N-Ph | b2 | H | b2 | a2 |
| 2-10-020 | N-Ph | N-Ph | b3 | H | b3 | a2 |
| 2-10-021 | N-Ph | N-Ph | H | H | H | a3 |
| 2-10-022 | N-Ph | N-Ph | t-bu | H | t-bu | a3 |
| 2-10-023 | N-Ph | N-Ph | methyl | H | methyl | a3 |
| 2-10-024 | N-Ph | N-Ph | b1 | H | b1 | a3 |
| 2-10-025 | N-Ph | N-Ph | b2 | H | b2 | a3 |
| 2-10-026 | N-Ph | N-Ph | b3 | H | b3 | a3 |
| 2-10-027 | N-Ph | N-Ph | H | H | H | a4 |
| 2-10-028 | N-Ph | N-Ph | t-bu | H | t-bu | a4 |
| 2-10-029 | N-Ph | N-Ph | methyl | H | methyl | a4 |
| 2-10-030 | N-Ph | N-Ph | b1 | H | b1 | a4 |
| 2-10-031 | N-Ph | N-Ph | b2 | H | b2 | a4 |
| 2-10-032 | N-Ph | N-Ph | b3 | H | b3 | a4 |
| 2-10-033 | N-Ph | N-Ph | H | H | H | a5 |
| 2-10-034 | N-Ph | N-Ph | t-bu | H | t-bu | a5 |
| 2-10-035 | N-Ph | N-Ph | methyl | H | methyl | a5 |
| 2-10-036 | N-Ph | N-Ph | b1 | H | b1 | a5 |
| 2-10-037 | N-Ph | N-Ph | b2 | H | b2 | a5 |
| 2-10-038 | N-Ph | N-Ph | b3 | H | b3 | a5 |
| 2-10-039 | N-Ph | N-Ph | H | H | H | a6 |
| 2-10-040 | N-Ph | N-Ph | t-bu | H | t-bu | a6 |
| 2-10-041 | N-Ph | N-Ph | methyl | H | methyl | a6 |
| 2-10-042 | N-Ph | N-Ph | b1 | H | b1 | a6 |
| 2-10-043 | N-Ph | N-Ph | b2 | H | b2 | a6 |
| 2-10-044 | N-Ph | N-Ph | b3 | H | b3 | a6 |
| 2-10-045 | N-Ph | N-Ph | H | H | H | b1 |
| 2-10-046 | N-Ph | N-Ph | t-bu | H | t-bu | b1 |
| 2-10-047 | N-Ph | N-Ph | methyl | H | methyl | b1 |
| 2-10-048 | N-Ph | N-Ph | b1 | H | b1 | b1 |
| 2-10-049 | N-Ph | N-Ph | b2 | H | b2 | b1 |
| 2-10-050 | N-Ph | N-Ph | b3 | H | b3 | b1 |
| 2-10-051 | N-Ph | N-Ph | H | H | H | b2 |
| 2-10-052 | N-Ph | N-Ph | t-bu | H | t-bu | b2 |
| 2-10-053 | N-Ph | N-Ph | methyl | H | methyl | b2 |
| 2-10-054 | N-Ph | N-Ph | b1 | H | b1 | b2 |
| 2-10-055 | N-Ph | N-Ph | b2 | H | b2 | b2 |
| 2-10-056 | N-Ph | N-Ph | b3 | H | b3 | b2 |
| 2-10-057 | N-Ph | N-Ph | H | H | H | b3 |
| 2-10-058 | N-Ph | N-Ph | t-bu | H | t-bu | b3 |
| 2-10-059 | N-Ph | N-Ph | methyl | H | methyl | b3 |
| 2-10-060 | N-Ph | N-Ph | b1 | H | b1 | b3 |
| 2-10-061 | N-Ph | N-Ph | b2 | H | b2 | b3 |
| 2-10-062 | N-Ph | N-Ph | b3 | H | b3 | b3 |
| 2-10-063 | N-Ph | N-Ph | H | H | H | b4 |
| 2-10-064 | N-Ph | N-Ph | t-bu | H | t-bu | b4 |
| 2-10-065 | N-Ph | N-Ph | methyl | H | methyl | b4 |
| 2-10-066 | N-Ph | N-Ph | b1 | H | b1 | b4 |
| 2-10-067 | N-Ph | N-Ph | b2 | H | b2 | b4 |
| 2-10-068 | N-Ph | N-Ph | b3 | H | b3 | b4 |
| 2-10-069 | N-Ph | N-Ph | H | H | H | b5 |
| 2-10-070 | N-Ph | N-Ph | t-bu | H | t-bu | b5 |
| 2-10-071 | N-Ph | N-Ph | methyl | H | methyl | b5 |
| 2-10-072 | N-Ph | N-Ph | b1 | H | b1 | b5 |
| 2-10-073 | N-Ph | N-Ph | b2 | H | b2 | b5 |
| 2-10-074 | N-Ph | N-Ph | b3 | H | b3 | b5 |
| 2-10-075 | N-Ph | N-Ph | H | H | H | b6 |
| 2-10-076 | N-Ph | N-Ph | t-bu | H | t-bu | b6 |
| 2-10-077 | N-Ph | N-Ph | methyl | H | methyl | b6 |
| 2-10-078 | N-Ph | N-Ph | b1 | H | b1 | b6 |
| 2-10-079 | N-Ph | N-Ph | b2 | H | b2 | b6 |
| 2-10-080 | N-Ph | N-Ph | b3 | H | b3 | b6 |
| 2-10-081 | N-Ph | N-Ph | H | H | H | b7 |
| 2-10-082 | N-Ph | N-Ph | t-bu | H | t-bu | b7 |
| 2-10-083 | N-Ph | N-Ph | methyl | H | methyl | b7 |
| 2-10-084 | N-Ph | N-Ph | b1 | H | b1 | b7 |
| 2-10-085 | N-Ph | N-Ph | b2 | H | b2 | b7 |
| 2-10-086 | N-Ph | N-Ph | b3 | H | b3 | b7 |
| 2-10-087 | N-Ph | N-Ph | H | H | H | b8 |
| 2-10-088 | N-Ph | N-Ph | t-bu | H | t-bu | b8 |
| 2-10-089 | N-Ph | N-Ph | methyl | H | methyl | b8 |
| 2-10-090 | N-Ph | N-Ph | b1 | H | b1 | b8 |
| 2-10-091 | N-Ph | N-Ph | b2 | H | b2 | b8 |
| 2-10-092 | N-Ph | N-Ph | b3 | H | b3 | b8 |
| 2-10-093 | N-Ph | N-Ph | H | H | H | b9 |
| 2-10-094 | N-Ph | N-Ph | t-bu | H | t-bu | b9 |
| 2-10-095 | N-Ph | N-Ph | methyl | H | methyl | b9 |
| 2-10-096 | N-Ph | N-Ph | b1 | H | b1 | b9 |
| 2-10-097 | N-Ph | N-Ph | b2 | H | b2 | b9 |
| 2-10-098 | N-Ph | N-Ph | b3 | H | b3 | b9 |
| 2-10-099 | N-Ph | N-Ph | H | H | H | b10 |
| 2-10-100 | N-Ph | N-Ph | t-bu | H | t-bu | b10 |
| 2-10-101 | N-Ph | N-Ph | methyl | H | methyl | b10 |
| 2-10-102 | N-Ph | N-Ph | b1 | H | b1 | b10 |
| 2-10-103 | N-Ph | N-Ph | b2 | H | b2 | b10 |
| 2-10-104 | N-Ph | N-Ph | b3 | H | b3 | b10 |
| 2-10-105 | N-tolyl | N-tolyl | H | H | H | H |
| 2-10-106 | N-tolyl | N-tolyl | t-bu | H | t-bu | H |
| 2-10-107 | N-tolyl | N-tolyl | methyl | H | methyl | H |
| 2-10-108 | N-tolyl | N-tolyl | t-bu | t-bu | t-bu | t-bu |
| 2-10-109 | N-tolyl | N-tolyl | methyl | methyl | methyl | methyl |
| 2-10-110 | N-tolyl | N-tolyl | b1 | H | b1 | H |
| 2-10-111 | N-tolyl | N-tolyl | b2 | H | b2 | H |
| 2-10-112 | N-tolyl | N-tolyl | b3 | H | b3 | H |
| 2-10-113 | N-tolyl | N-tolyl | H | H | H | a1 |
| 2-10-114 | N-tolyl | N-tolyl | t-bu | H | t-bu | a1 |
| 2-10-115 | N-tolyl | N-tolyl | methyl | H | methyl | a1 |
| 2-10-116 | N-tolyl | N-tolyl | b1 | H | b1 | a1 |
| 2-10-117 | N-tolyl | N-tolyl | b2 | H | b2 | a1 |
| 2-10-118 | N-tolyl | N-tolyl | b3 | H | b3 | a1 |
| 2-10-119 | N-tolyl | N-tolyl | H | H | H | a2 |
| 2-10-120 | N-tolyl | N-tolyl | t-bu | H | t-bu | a2 |
| 2-10-121 | N-tolyl | N-tolyl | methyl | H | methyl | a2 |
| 2-10-122 | N-tolyl | N-tolyl | b1 | H | b1 | a2 |
| 2-10-123 | N-tolyl | N-tolyl | b2 | H | b2 | a2 |
| 2-10-124 | N-tolyl | N-tolyl | b3 | H | b3 | a2 |
| 2-10-125 | N-tolyl | N-tolyl | H | H | H | a3 |
| 2-10-126 | N-tolyl | N-tolyl | t-bu | H | t-bu | a3 |
| 2-10-127 | N-tolyl | N-tolyl | methyl | H | methyl | a3 |
| 2-10-128 | N-tolyl | N-tolyl | b1 | H | b1 | a3 |
| 2-10-129 | N-tolyl | N-tolyl | b2 | H | b2 | a3 |
| 2-10-130 | N-tolyl | N-tolyl | b3 | H | b3 | a3 |
| 2-10-131 | N-tolyl | N-tolyl | H | H | H | a4 |
| 2-10-132 | N-tolyl | N-tolyl | t-bu | H | t-bu | a4 |
| 2-10-133 | N-tolyl | N-tolyl | methyl | H | methyl | a4 |
| 2-10-134 | N-tolyl | N-tolyl | b1 | H | b1 | a4 |
| 2-10-135 | N-tolyl | N-tolyl | b2 | H | b2 | a4 |
| 2-10-136 | N-tolyl | N-tolyl | b3 | H | b3 | a4 |
| 2-10-137 | N-tolyl | N-tolyl | H | H | H | a5 |
| 2-10-138 | N-tolyl | N-tolyl | t-bu | H | t-bu | a5 |
| 2-10-139 | N-tolyl | N-tolyl | methyl | H | methyl | a5 |
| 2-10-140 | N-tolyl | N-tolyl | b1 | H | b1 | a5 |
| 2-10-141 | N-tolyl | N-tolyl | b2 | H | b2 | a5 |
| 2-10-142 | N-tolyl | N-tolyl | b3 | H | b3 | a5 |
| 2-10-143 | N-tolyl | N-tolyl | H | H | H | a6 |
| 2-10-144 | N-tolyl | N-tolyl | t-bu | H | t-bu | a6 |
| 2-10-145 | N-tolyl | N-tolyl | methyl | H | methyl | a6 |
| 2-10-146 | N-tolyl | N-tolyl | b1 | H | b1 | a6 |
| 2-10-147 | N-tolyl | N-tolyl | b2 | H | b2 | a6 |
| 2-10-148 | N-tolyl | N-tolyl | b3 | H | b3 | a6 |
| 2-10-149 | N-tolyl | N-tolyl | H | H | H | b1 |
| 2-10-150 | N-tolyl | N-tolyl | t-bu | H | t-bu | b1 |
| 2-10-151 | N-tolyl | N-tolyl | methyl | H | methyl | b1 |
| 2-10-152 | N-tolyl | N-tolyl | b1 | H | b1 | b1 |
| 2-10-153 | N-tolyl | N-tolyl | b2 | H | b2 | b1 |
| 2-10-154 | N-tolyl | N-tolyl | b3 | H | b3 | b1 |
| 2-10-155 | N-tolyl | N-tolyl | H | H | H | b2 |
| 2-10-156 | N-tolyl | N-tolyl | t-bu | H | t-bu | b2 |
| 2-10-157 | N-tolyl | N-tolyl | methyl | H | methyl | b2 |
| 2-10-158 | N-tolyl | N-tolyl | b1 | H | b1 | b2 |
| 2-10-159 | N-tolyl | N-tolyl | b2 | H | b2 | b2 |
| 2-10-160 | N-tolyl | N-tolyl | b3 | H | b3 | b2 |
| 2-10-161 | N-tolyl | N-tolyl | H | H | H | b3 |
| 2-10-162 | N-tolyl | N-tolyl | t-bu | H | t-bu | b3 |
| 2-10-163 | N-tolyl | N-tolyl | methyl | H | methyl | b3 |
| 2-10-164 | N-tolyl | N-tolyl | b1 | H | b1 | b3 |
| 2-10-165 | N-tolyl | N-tolyl | b2 | H | b2 | b3 |
| 2-10-166 | N-tolyl | N-tolyl | b3 | H | b3 | b3 |
| 2-10-167 | N-tolyl | N-tolyl | H | H | H | b4 |
| 2-10-168 | N-tolyl | N-tolyl | t-bu | H | t-bu | b4 |
| 2-10-169 | N-tolyl | N-tolyl | methyl | H | methyl | b4 |
| 2-10-170 | N-tolyl | N-tolyl | b1 | H | b1 | b4 |
| 2-10-171 | N-tolyl | N-tolyl | b2 | H | b2 | b4 |
| 2-10-172 | N-tolyl | N-tolyl | b3 | H | b3 | b4 |
| 2-10-173 | N-tolyl | N-tolyl | H | H | H | b5 |
| 2-10-174 | N-tolyl | N-tolyl | t-bu | H | t-bu | b5 |
| 2-10-175 | N-tolyl | N-tolyl | methyl | H | methyl | b5 |
| 2-10-176 | N-tolyl | N-tolyl | b1 | H | b1 | b5 |
| 2-10-177 | N-tolyl | N-tolyl | b2 | H | b2 | b5 |
| 2-10-178 | N-tolyl | N-tolyl | b3 | H | b3 | b5 |
| 2-10-179 | N-tolyl | N-tolyl | H | H | H | b6 |
| 2-10-180 | N-tolyl | N-tolyl | t-bu | H | t-bu | b6 |
| 2-10-181 | N-tolyl | N-tolyl | methyl | H | methyl | b6 |
| 2-10-182 | N-tolyl | N-tolyl | b1 | H | b1 | b6 |
| 2-10-183 | N-tolyl | N-tolyl | b2 | H | b2 | b6 |
| 2-10-184 | N-tolyl | N-tolyl | b3 | H | b3 | b6 |
| 2-10-185 | N-tolyl | N-tolyl | H | H | H | b7 |
| 2-10-186 | N-tolyl | N-tolyl | t-bu | H | t-bu | b7 |
| 2-10-187 | N-tolyl | N-tolyl | methyl | H | methyl | b7 |
| 2-10-188 | N-tolyl | N-tolyl | b1 | H | b1 | b7 |
| 2-10-189 | N-tolyl | N-tolyl | b2 | H | b2 | b7 |
| 2-10-190 | N-tolyl | N-tolyl | b3 | H | b3 | b7 |
| 2-10-191 | N-tolyl | N-tolyl | H | H | H | b8 |
| 2-10-192 | N-tolyl | N-tolyl | t-bu | H | t-bu | b8 |
| 2-10-193 | N-tolyl | N-tolyl | methyl | H | methyl | b8 |
| 2-10-194 | N-tolyl | N-tolyl | b1 | H | b1 | b8 |
| 2-10-195 | N-tolyl | N-tolyl | b2 | H | b2 | b8 |
| 2-10-196 | N-tolyl | N-tolyl | b3 | H | b3 | b8 |
| 2-10-197 | N-tolyl | N-tolyl | H | H | H | b9 |
| 2-10-198 | N-tolyl | N-tolyl | t-bu | H | t-bu | b9 |
| 2-10-199 | N-tolyl | N-tolyl | methyl | H | methyl | b9 |
| 2-10-200 | N-tolyl | N-tolyl | b1 | H | b1 | b9 |
| 2-10-201 | N-tolyl | N-tolyl | b2 | H | b2 | b9 |
| 2-10-202 | N-tolyl | N-tolyl | b3 | H | b3 | b9 |
| 2-10-203 | N-tolyl | N-tolyl | H | H | H | b10 |
| 2-10-204 | N-tolyl | N-tolyl | t-bu | H | t-bu | b10 |
| 2-10-205 | N-tolyl | N-tolyl | methyl | H | methyl | b10 |
| 2-10-206 | N-tolyl | N-tolyl | b1 | H | b1 | b10 |
| 2-10-207 | N-tolyl | N-tolyl | b2 | H | b2 | b10 |
| 2-10-208 | N-tolyl | N-tolyl | b3 | H | b3 | b10 |
| 2-10-209 | N-Biph | N-Biph | H | H | H | H |
| 2-10-210 | N-Biph | N-Biph | t-bu | H | t-bu | H |
| 2-10-211 | N-Biph | N-Biph | methyl | H | methyl | H |
| 2-10-212 | N-Biph | N-Biph | t-bu | t-bu | t-bu | t-bu |
| 2-10-213 | N-Biph | N-Biph | methyl | methyl | methyl | methyl |
| 2-10-214 | N-Biph | N-Biph | b1 | H | b1 | H |
| 2-10-215 | N-Biph | N-Biph | b2 | H | b2 | H |
| 2-10-216 | N-Biph | N-Biph | b3 | H | b3 | H |
| 2-10-217 | N-Biph | N-Biph | H | H | H | a1 |
| 2-10-218 | N-Biph | N-Biph | t-bu | H | t-bu | a1 |
| 2-10-219 | N-Biph | N-Biph | methyl | H | methyl | a1 |
| 2-10-220 | N-Biph | N-Biph | b1 | H | b1 | a1 |
| 2-10-221 | N-Biph | N-Biph | b2 | H | b2 | a1 |
| 2-10-222 | N-Biph | N-Biph | b3 | H | b3 | a1 |
| 2-10-223 | N-Biph | N-Biph | H | H | H | a2 |
| 2-10-224 | N-Biph | N-Biph | t-bu | H | t-bu | a2 |
| 2-10-225 | N-Biph | N-Biph | methyl | H | methyl | a2 |
| 2-10-226 | N-Biph | N-Biph | b1 | H | b1 | a2 |
| 2-10-227 | N-Biph | N-Biph | b2 | H | b2 | a2 |
| 2-10-228 | N-Biph | N-Biph | b3 | H | b3 | a2 |
| 2-10-229 | N-Biph | N-Biph | H | H | H | a3 |
| 2-10-230 | N-Biph | N-Biph | t-bu | H | t-bu | a3 |
| 2-10-231 | N-Biph | N-Biph | methyl | H | methyl | a3 |
| 2-10-232 | N-Biph | N-Biph | b1 | H | b1 | a3 |
| 2-10-233 | N-Biph | N-Biph | b2 | H | b2 | a3 |
| 2-10-234 | N-Biph | N-Biph | b3 | H | b3 | a3 |
| 2-10-235 | N-Biph | N-Biph | H | H | H | a4 |
| 2-10-236 | N-Biph | N-Biph | t-bu | H | t-bu | a4 |
| 2-10-237 | N-Biph | N-Biph | methyl | H | methyl | a4 |
| 2-10-238 | N-Biph | N-Biph | b1 | H | b1 | a4 |
| 2-10-239 | N-Biph | N-Biph | b2 | H | b2 | a4 |
| 2-10-240 | N-Biph | N-Biph | b3 | H | b3 | a4 |
| 2-10-241 | N-Biph | N-Biph | H | H | H | a5 |
| 2-10-242 | N-Biph | N-Biph | t-bu | H | t-bu | a5 |
| 2-10-243 | N-Biph | N-Biph | methyl | H | methyl | a5 |
| 2-10-244 | N-Biph | N-Biph | b1 | H | b1 | a5 |
| 2-10-245 | N-Biph | N-Biph | b2 | H | b2 | a5 |
| 2-10-246 | N-Biph | N-Biph | b3 | H | b3 | a5 |
| 2-10-247 | N-Biph | N-Biph | H | H | H | a6 |
| 2-10-248 | N-Biph | N-Biph | t-bu | H | t-bu | a6 |
| 2-10-249 | N-Biph | N-Biph | methyl | H | methyl | a6 |
| 2-10-250 | N-Biph | N-Biph | b1 | H | b1 | a6 |
| 2-10-251 | N-Biph | N-Biph | b2 | H | b2 | a6 |
| 2-10-252 | N-Biph | N-Biph | b3 | H | b3 | a6 |
| 2-10-253 | N-Biph | N-Biph | H | H | H | b1 |
| 2-10-254 | N-Biph | N-Biph | t-bu | H | t-bu | b1 |
| 2-10-255 | N-Biph | N-Biph | methyl | H | methyl | b1 |
| 2-10-256 | N-Biph | N-Biph | b1 | H | b1 | b1 |
| 2-10-257 | N-Biph | N-Biph | b2 | H | b2 | b1 |
| 2-10-258 | N-Biph | N-Biph | b3 | H | b3 | b1 |
| 2-10-259 | N-Biph | N-Biph | H | H | H | b2 |
| 2-10-260 | N-Biph | N-Biph | t-bu | H | t-bu | b2 |
| 2-10-261 | N-Biph | N-Biph | methyl | H | methyl | b2 |
| 2-10-262 | N-Biph | N-Biph | b1 | H | b1 | b2 |
| 2-10-263 | N-Biph | N-Biph | b2 | H | b2 | b2 |
| 2-10-264 | N-Biph | N-Biph | b3 | H | b3 | b2 |
| 2-10-265 | N-Biph | N-Biph | H | H | H | b3 |
| 2-10-266 | N-Biph | N-Biph | t-bu | H | t-bu | b3 |
| 2-10-267 | N-Biph | N-Biph | methyl | H | methyl | b3 |
| 2-10-268 | N-Biph | N-Biph | b1 | H | b1 | b3 |
| 2-10-269 | N-Biph | N-Biph | b2 | H | b2 | b3 |
| 2-10-270 | N-Biph | N-Biph | b3 | H | b3 | b3 |
| 2-10-271 | N-Biph | N-Biph | H | H | H | b4 |
| 2-10-272 | N-Biph | N-Biph | t-bu | H | t-bu | b4 |
| 2-10-273 | N-Biph | N-Biph | methyl | H | methyl | b4 |
| 2-10-274 | N-Biph | N-Biph | b1 | H | b1 | b4 |
| 2-10-275 | N-Biph | N-Biph | b2 | H | b2 | b4 |
| 2-10-276 | N-Biph | N-Biph | b3 | H | b3 | b4 |
| 2-10-277 | N-Biph | N-Biph | H | H | H | b5 |
| 2-10-278 | N-Biph | N-Biph | t-bu | H | t-bu | b5 |
| 2-10-279 | N-Biph | N-Biph | methyl | H | methyl | b5 |
| 2-10-280 | N-Biph | N-Biph | b1 | H | b1 | b5 |
| 2-10-281 | N-Biph | N-Biph | b2 | H | b2 | b5 |
| 2-10-282 | N-Biph | N-Biph | b3 | H | b3 | b5 |
| 2-10-283 | N-Biph | N-Biph | H | H | H | b6 |
| 2-10-284 | N-Biph | N-Biph | t-bu | H | t-bu | b6 |
| 2-10-285 | N-Biph | N-Biph | methyl | H | methyl | b6 |
| 2-10-286 | N-Biph | N-Biph | b1 | H | b1 | b6 |
| 2-10-287 | N-Biph | N-Biph | b2 | H | b2 | b6 |
| 2-10-288 | N-Biph | N-Biph | b3 | H | b3 | b6 |
| 2-10-289 | N-Biph | N-Biph | H | H | H | b7 |
| 2-10-290 | N-Biph | N-Biph | t-bu | H | t-bu | b7 |
| 2-10-291 | N-Biph | N-Biph | methyl | H | methyl | b7 |
| 2-10-292 | N-Biph | N-Biph | b1 | H | b1 | b7 |
| 2-10-293 | N-Biph | N-Biph | b2 | H | b2 | b7 |
| 2-10-294 | N-Biph | N-Biph | b3 | H | b3 | b7 |
| 2-10-295 | N-Biph | N-Biph | H | H | H | b8 |
| 2-10-296 | N-Biph | N-Biph | t-bu | H | t-bu | b8 |
| 2-10-297 | N-Biph | N-Biph | methyl | H | methyl | b8 |
| 2-10-298 | N-Biph | N-Biph | b1 | H | b1 | b8 |
| 2-10-299 | N-Biph | N-Biph | b2 | H | b2 | b8 |
| 2-10-300 | N-Biph | N-Biph | b3 | H | b3 | b8 |
| 2-10-301 | N-Biph | N-Biph | H | H | H | b9 |
| 2-10-302 | N-Biph | N-Biph | t-bu | H | t-bu | b9 |
| 2-10-303 | N-Biph | N-Biph | methyl | H | methyl | b9 |
| 2-10-304 | N-Biph | N-Biph | b1 | H | b1 | b9 |
| 2-10-305 | N-Biph | N-Biph | b2 | H | b2 | b9 |
| 2-10-306 | N-Biph | N-Biph | b3 | H | b3 | b9 |
| 2-10-307 | N-Biph | N-Biph | H | H | H | b10 |
| 2-10-308 | N-Biph | N-Biph | t-bu | H | t-bu | b10 |
| 2-10-309 | N-Biph | N-Biph | methyl | H | methyl | b10 |
| 2-10-310 | N-Biph | N-Biph | b1 | H | b1 | b10 |
| 2-10-311 | N-Biph | N-Biph | b2 | H | b2 | b10 |
| 2-10-312 | N-Biph | N-Biph | b3 | H | b3 | b10 |
| 2-10-313 | O | N-Ph | H | H | H | H |
| 2-10-314 | O | N-Ph | t-bu | H | t-bu | H |
| 2-10-315 | O | N-Ph | methyl | H | methyl | H |
| 2-10-316 | O | N-Ph | t-bu | t-bu | t-bu | t-bu |
| 2-10-317 | O | N-Ph | methyl | methyl | methyl | methyl |
| 2-10-318 | O | N-Ph | b1 | H | b1 | H |
| 2-10-319 | O | N-Ph | b2 | H | b2 | H |
| 2-10-320 | O | N-Ph | b3 | H | b3 | H |
| 2-10-321 | O | N-Ph | H | H | H | a1 |
| 2-10-322 | O | N-Ph | t-bu | H | t-bu | a1 |
| 2-10-323 | O | N-Ph | methyl | H | methyl | a1 |
| 2-10-324 | O | N-Ph | b1 | H | b1 | a1 |
| 2-10-325 | O | N-Ph | b2 | H | b2 | a1 |
| 2-10-326 | O | N-Ph | b3 | H | b3 | a1 |
| 2-10-327 | O | N-Ph | H | H | H | a2 |
| 2-10-328 | O | N-Ph | t-bu | H | t-bu | a2 |
| 2-10-329 | O | N-Ph | methyl | H | methyl | a2 |
| 2-10-330 | O | N-Ph | b1 | H | b1 | a2 |
| 2-10-331 | O | N-Ph | b2 | H | b2 | a2 |
| 2-10-332 | O | N-Ph | b3 | H | b3 | a2 |
| 2-10-333 | O | N-Ph | H | H | H | a3 |
| 2-10-334 | O | N-Ph | t-bu | H | t-bu | a3 |
| 2-10-335 | O | N-Ph | methyl | H | methyl | a3 |
| 2-10-336 | O | N-Ph | b1 | H | b1 | a3 |
| 2-10-337 | O | N-Ph | b2 | H | b2 | a3 |
| 2-10-338 | O | N-Ph | b3 | H | b3 | a3 |
| 2-10-339 | O | N-Ph | H | H | H | a4 |
| 2-10-340 | O | N-Ph | t-bu | H | t-bu | a4 |
| 2-10-341 | O | N-Ph | methyl | H | methyl | a4 |
| 2-10-342 | O | N-Ph | b1 | H | b1 | a4 |
| 2-10-343 | O | N-Ph | b2 | H | b2 | a4 |
| 2-10-344 | O | N-Ph | b3 | H | b3 | a4 |
| 2-10-345 | O | N-Ph | H | H | H | a5 |
| 2-10-346 | O | N-Ph | t-bu | H | t-bu | a5 |
| 2-10-347 | O | N-Ph | methyl | H | methyl | a5 |
| 2-10-348 | O | N-Ph | b1 | H | b1 | a5 |
| 2-10-349 | O | N-Ph | b2 | H | b2 | a5 |
| 2-10-350 | O | N-Ph | b3 | H | b3 | a5 |
| 2-10-351 | O | N-Ph | H | H | H | a6 |
| 2-10-352 | O | N-Ph | t-bu | H | t-bu | a6 |
| 2-10-353 | O | N-Ph | methyl | H | methyl | a6 |
| 2-10-354 | O | N-Ph | b1 | H | b1 | a6 |
| 2-10-355 | O | N-Ph | b2 | H | b2 | a6 |
| 2-10-356 | O | N-Ph | b3 | H | b3 | a6 |
| 2-10-357 | O | N-Ph | H | H | H | b1 |
| 2-10-358 | O | N-Ph | t-bu | H | t-bu | b1 |
| 2-10-359 | O | N-Ph | methyl | H | methyl | b1 |
| 2-10-360 | O | N-Ph | b1 | H | b1 | b1 |
| 2-10-361 | O | N-Ph | b2 | H | b2 | b1 |
| 2-10-362 | O | N-Ph | b3 | H | b3 | b1 |
| 2-10-363 | O | N-Ph | H | H | H | b2 |
| 2-10-364 | O | N-Ph | t-bu | H | t-bu | b2 |
| 2-10-365 | O | N-Ph | methyl | H | methyl | b2 |
| 2-10-366 | O | N-Ph | b1 | H | b1 | b2 |
| 2-10-367 | O | N-Ph | b2 | H | b2 | b2 |
| 2-10-368 | O | N-Ph | b3 | H | b3 | b2 |
| 2-10-369 | O | N-Ph | H | H | H | b3 |
| 2-10-370 | O | N-Ph | t-bu | H | t-bu | b3 |
| 2-10-371 | O | N-Ph | methyl | H | methyl | b3 |
| 2-10-372 | O | N-Ph | b1 | H | b1 | b3 |
| 2-10-373 | O | N-Ph | b2 | H | b2 | b3 |
| 2-10-374 | O | N-Ph | b3 | H | b3 | b3 |
| 2-10-375 | O | N-Ph | H | H | H | b4 |
| 2-10-376 | O | N-Ph | t-bu | H | t-bu | b4 |
| 2-10-377 | O | N-Ph | methyl | H | methyl | b4 |
| 2-10-378 | O | N-Ph | b1 | H | b1 | b4 |
| 2-10-379 | O | N-Ph | b2 | H | b2 | b4 |
| 2-10-380 | O | N-Ph | b3 | H | b3 | b4 |
| 2-10-381 | O | N-Ph | H | H | H | b5 |
| 2-10-382 | O | N-Ph | t-bu | H | t-bu | b5 |
| 2-10-383 | O | N-Ph | methyl | H | methyl | b5 |
| 2-10-384 | O | N-Ph | b1 | H | b1 | b5 |
| 2-10-385 | O | N-Ph | b2 | H | b2 | b5 |
| 2-10-386 | O | N-Ph | b3 | H | b3 | b5 |
| 2-10-387 | O | N-Ph | H | H | H | b6 |
| 2-10-388 | O | N-Ph | t-bu | H | t-bu | b6 |
| 2-10-389 | O | N-Ph | methyl | H | methyl | b6 |
| 2-10-390 | O | N-Ph | b1 | H | b1 | b6 |
| 2-10-391 | O | N-Ph | b2 | H | b2 | b6 |
| 2-10-392 | O | N-Ph | b3 | H | b3 | b6 |
| 2-10-393 | O | N-Ph | H | H | H | b7 |
| 2-10-394 | O | N-Ph | t-bu | H | t-bu | b7 |
| 2-10-395 | O | N-Ph | methyl | H | methyl | b7 |
| 2-10-396 | O | N-Ph | b1 | H | b1 | b7 |
| 2-10-397 | O | N-Ph | b2 | H | b2 | b7 |
| 2-10-398 | O | N-Ph | b3 | H | b3 | b7 |
| 2-10-399 | O | N-Ph | H | H | H | b8 |
| 2-10-400 | O | N-Ph | t-bu | H | t-bu | b8 |
| 2-10-401 | O | N-Ph | methyl | H | methyl | b8 |
| 2-10-402 | O | N-Ph | b1 | H | b1 | b8 |
| 2-10-403 | O | N-Ph | b2 | H | b2 | b8 |
| 2-10-404 | O | N-Ph | b3 | H | b3 | b8 |
| 2-10-405 | O | N-Ph | H | H | H | b9 |
| 2-10-406 | O | N-Ph | t-bu | H | t-bu | b9 |
| 2-10-407 | O | N-Ph | methyl | H | methyl | b9 |
| 2-10-408 | O | N-Ph | b1 | H | b1 | b9 |
| 2-10-409 | O | N-Ph | b2 | H | b2 | b9 |
| 2-10-410 | O | N-Ph | b3 | H | b3 | b9 |
| 2-10-411 | O | N-Ph | H | H | H | b10 |
| 2-10-412 | O | N-Ph | t-bu | H | t-bu | b10 |
| 2-10-413 | O | N-Ph | methyl | H | methyl | b10 |
| 2-10-414 | O | N-Ph | b1 | H | b1 | b10 |
| 2-10-415 | O | N-Ph | b2 | H | b2 | b10 |
| 2-10-416 | O | N-Ph | b3 | H | b3 | b10 |
| 2-10-417 | S | N-Ph | H | H | H | H |
| 2-10-418 | S | N-Ph | t-bu | H | t-bu | H |
| 2-10-419 | S | N-Ph | methyl | H | methyl | H |
| 2-10-420 | S | N-Ph | t-bu | t-bu | t-bu | t-bu |
| 2-10-421 | S | N-Ph | methyl | methyl | methyl | methyl |
| 2-10-422 | S | N-Ph | b1 | H | b1 | H |
| 2-10-423 | S | N-Ph | b2 | H | b2 | H |
| 2-10-424 | S | N-Ph | b3 | H | b3 | H |
| 2-10-425 | S | N-Ph | H | H | H | a1 |
| 2-10-426 | S | N-Ph | t-bu | H | t-bu | a1 |
| 2-10-427 | S | N-Ph | methyl | H | methyl | a1 |
| 2-10-428 | S | N-Ph | b1 | H | b1 | a1 |
| 2-10-429 | S | N-Ph | b2 | H | b2 | a1 |
| 2-10-430 | S | N-Ph | b3 | H | b3 | a1 |
| 2-10-431 | S | N-Ph | H | H | H | a2 |
| 2-10-432 | S | N-Ph | t-bu | H | t-bu | a2 |
| 2-10-433 | S | N-Ph | methyl | H | methyl | a2 |
| 2-10-434 | S | N-Ph | b1 | H | b1 | a2 |
| 2-10-435 | S | N-Ph | b2 | H | b2 | a2 |
| 2-10-436 | S | N-Ph | b3 | H | b3 | a2 |
| 2-10-437 | S | N-Ph | H | H | H | a3 |
| 2-10-438 | S | N-Ph | t-bu | H | t-bu | a3 |
| 2-10-439 | S | N-Ph | methyl | H | methyl | a3 |
| 2-10-440 | S | N-Ph | b1 | H | b1 | a3 |
| 2-10-441 | S | N-Ph | b2 | H | b2 | a3 |
| 2-10-442 | S | N-Ph | b3 | H | b3 | a3 |
| 2-10-443 | S | N-Ph | H | H | H | a4 |
| 2-10-444 | S | N-Ph | t-bu | H | t-bu | a4 |
| 2-10-445 | S | N-Ph | methyl | H | methyl | a4 |
| 2-10-446 | S | N-Ph | b1 | H | b1 | a4 |
| 2-10-447 | S | N-Ph | b2 | H | b2 | a4 |
| 2-10-448 | S | N-Ph | b3 | H | b3 | a4 |
| 2-10-449 | S | N-Ph | H | H | H | a5 |
| 2-10-450 | S | N-Ph | t-bu | H | t-bu | a5 |
| 2-10-451 | S | N-Ph | methyl | H | methyl | a5 |
| 2-10-452 | S | N-Ph | b1 | H | b1 | a5 |
| 2-10-453 | S | N-Ph | b2 | H | b2 | a5 |
| 2-10-454 | S | N-Ph | b3 | H | b3 | a5 |
| 2-10-455 | S | N-Ph | H | H | H | a6 |
| 2-10-456 | S | N-Ph | t-bu | H | t-bu | a6 |
| 2-10-457 | S | N-Ph | methyl | H | methyl | a6 |
| 2-10-458 | S | N-Ph | b1 | H | b1 | a6 |
| 2-10-459 | S | N-Ph | b2 | H | b2 | a6 |
| 2-10-460 | S | N-Ph | b3 | H | b3 | a6 |
| 2-10-461 | S | N-Ph | H | H | H | b1 |
| 2-10-462 | S | N-Ph | t-bu | H | t-bu | b1 |
| 2-10-463 | S | N-Ph | methyl | H | methyl | b1 |
| 2-10-464 | S | N-Ph | b1 | H | b1 | b1 |
| 2-10-465 | S | N-Ph | b2 | H | b2 | b1 |
| 2-10-466 | S | N-Ph | b3 | H | b3 | b1 |
| 2-10-467 | S | N-Ph | H | H | H | b2 |
| 2-10-468 | S | N-Ph | t-bu | H | t-bu | b2 |
| 2-10-469 | S | N-Ph | methyl | H | methyl | b2 |
| 2-10-470 | S | N-Ph | b1 | H | b1 | b2 |
| 2-10-471 | S | N-Ph | b2 | H | b2 | b2 |
| 2-10-472 | S | N-Ph | b3 | H | b3 | b2 |
| 2-10-473 | S | N-Ph | H | H | H | b3 |
| 2-10-474 | S | N-Ph | t-bu | H | t-bu | b3 |
| 2-10-475 | S | N-Ph | methyl | H | methyl | b3 |
| 2-10-476 | S | N-Ph | b1 | H | b1 | b3 |
| 2-10-477 | S | N-Ph | b2 | H | b2 | b3 |
| 2-10-478 | S | N-Ph | b3 | H | b3 | b3 |
| 2-10-479 | S | N-Ph | H | H | H | b4 |
| 2-10-480 | S | N-Ph | t-bu | H | t-bu | b4 |
| 2-10-481 | S | N-Ph | methyl | H | methyl | b4 |
| 2-10-482 | S | N-Ph | b1 | H | b1 | b4 |
| 2-10-483 | S | N-Ph | b2 | H | b2 | b4 |
| 2-10-484 | S | N-Ph | b3 | H | b3 | b4 |
| 2-10-485 | S | N-Ph | H | H | H | b5 |
| 2-10-486 | S | N-Ph | t-bu | H | t-bu | b5 |
| 2-10-487 | S | N-Ph | methyl | H | methyl | b5 |
| 2-10-488 | S | N-Ph | b1 | H | b1 | b5 |
| 2-10-489 | S | N-Ph | b2 | H | b2 | b5 |
| 2-10-490 | S | N-Ph | b3 | H | b3 | b5 |
| 2-10-491 | S | N-Ph | H | H | H | b6 |
| 2-10-492 | S | N-Ph | t-bu | H | t-bu | b6 |
| 2-10-493 | S | N-Ph | methyl | H | methyl | b6 |
| 2-10-494 | S | N-Ph | b1 | H | b1 | b6 |
| 2-10-495 | S | N-Ph | b2 | H | b2 | b6 |
| 2-10-496 | S | N-Ph | b3 | H | b3 | b6 |
| 2-10-497 | S | N-Ph | H | H | H | b7 |
| 2-10-498 | S | N-Ph | t-bu | H | t-bu | b7 |
| 2-10-499 | S | N-Ph | methyl | H | methyl | b7 |
| 2-10-500 | S | N-Ph | b1 | H | b1 | b7 |
| 2-10-501 | S | N-Ph | b2 | H | b2 | b7 |
| 2-10-502 | S | N-Ph | b3 | H | b3 | b7 |
| 2-10-503 | S | N-Ph | H | H | H | b8 |
| 2-10-504 | S | N-Ph | t-bu | H | t-bu | b8 |
| 2-10-505 | S | N-Ph | methyl | H | methyl | b8 |
| 2-10-506 | S | N-Ph | b1 | H | b1 | b8 |
| 2-10-507 | S | N-Ph | b2 | H | b2 | b8 |
| 2-10-508 | S | N-Ph | b3 | H | b3 | b8 |
| 2-10-509 | S | N-Ph | H | H | H | b9 |
| 2-10-510 | S | N-Ph | t-bu | H | t-bu | b9 |
| 2-10-511 | S | N-Ph | methyl | H | methyl | b9 |
| 2-10-512 | S | N-Ph | b1 | H | b1 | b9 |
| 2-10-513 | S | N-Ph | b2 | H | b2 | b9 |
| 2-10-514 | S | N-Ph | b3 | H | b3 | b9 |
| 2-10-515 | S | N-Ph | H | H | H | b10 |
| 2-10-516 | S | N-Ph | t-bu | H | t-bu | b10 |
| 2-10-517 | S | N-Ph | methyl | H | methyl | b10 |
| 2-10-518 | S | N-Ph | b1 | H | b1 | b10 |
| 2-10-519 | S | N-Ph | b2 | H | b2 | b10 |
| 2-10-520 | S | N-Ph | b3 | H | b3 | b10 |

Group 2-11
The compounds are represented by the formula 2-11 below, wherein CY₂ is a benzene group, X₁ is B, and Y1, Y2, R1a, R1c, R3a, (R2)a2 are each shown in the table 11 below: 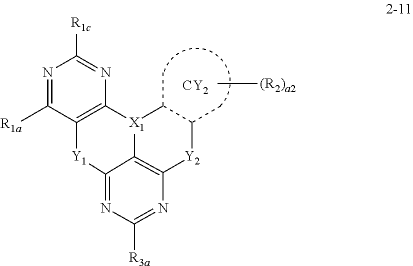
TABLE 11

| Compound | | | | | | |
|---|---|---|---|---|---|---|
| No. | Y1 | Y2 | R1a | R1c | R3a | (R2)a2 |
| 2-11-001 | N-Ph | N-Ph | H | H | H | H |
| 2-11-002 | N-Ph | N-Ph | t-bu | H | t-bu | H |
| 2-11-003 | N-Ph | N-Ph | methyl | H | methyl | H |
| 2-11-004 | N-Ph | N-Ph | t-bu | t-bu | t-bu | t-bu |
| 2-11-005 | N-Ph | N-Ph | methyl | methyl | methyl | methyl |
| 2-11-006 | N-Ph | N-Ph | b1 | H | b1 | H |
| 2-11-007 | N-Ph | N-Ph | b2 | H | b2 | H |
| 2-11-008 | N-Ph | N-Ph | b3 | H | b3 | H |
| 2-11-009 | N-Ph | N-Ph | H | H | H | a1 |
| 2-11-010 | N-Ph | N-Ph | t-bu | t-bu | t-bu | a1 |
| 2-11-011 | N-Ph | N-Ph | methyl | methyl | methyl | a1 |
| 2-11-012 | N-Ph | N-Ph | b1 | b1 | b1 | a1 |
| 2-11-013 | N-Ph | N-Ph | b2 | b2 | b2 | a1 |
| 2-11-014 | N-Ph | N-Ph | b3 | b3 | b3 | a1 |
| 2-11-015 | N-Ph | N-Ph | H | H | H | a2 |
| 2-11-016 | N-Ph | N-Ph | t-bu | t-bu | t-bu | a2 |
| 2-11-017 | N-Ph | N-Ph | methyl | methyl | methyl | a2 |
| 2-11-018 | N-Ph | N-Ph | b1 | b1 | b1 | a2 |
| 2-11-019 | N-Ph | N-Ph | b2 | b2 | b2 | a2 |
| 2-11-020 | N-Ph | N-Ph | b3 | b3 | b3 | a2 |
| 2-11-021 | N-Ph | N-Ph | H | H | H | a3 |
| 2-11-022 | N-Ph | N-Ph | t-bu | t-bu | t-bu | a3 |
| 2-11-023 | N-Ph | N-Ph | methyl | methyl | methyl | a3 |
| 2-11-024 | N-Ph | N-Ph | b1 | b1 | b1 | a3 |
| 2-11-025 | N-Ph | N-Ph | b2 | b2 | b2 | a3 |
| 2-11-026 | N-Ph | N-Ph | b3 | b3 | b3 | a3 |
| 2-11-027 | N-Ph | N-Ph | H | H | H | a4 |
| 2-11-028 | N-Ph | N-Ph | t-bu | t-bu | t-bu | a4 |
| 2-11-029 | N-Ph | N-Ph | methyl | methyl | methyl | a4 |
| 2-11-030 | N-Ph | N-Ph | b1 | b1 | b1 | a4 |
| 2-11-031 | N-Ph | N-Ph | b2 | b2 | b2 | a4 |
| 2-11-032 | N-Ph | N-Ph | b3 | b3 | b3 | a4 |
| 2-11-033 | N-Ph | N-Ph | H | H | H | a5 |
| 2-11-034 | N-Ph | N-Ph | t-bu | t-bu | t-bu | a5 |
| 2-11-035 | N-Ph | N-Ph | methyl | methyl | methyl | a5 |
| 2-11-036 | N-Ph | N-Ph | b1 | b1 | b1 | a5 |
| 2-11-037 | N-Ph | N-Ph | b2 | b2 | b2 | a5 |
| 2-11-038 | N-Ph | N-Ph | b3 | b3 | b3 | a5 |
| 2-11-039 | N-Ph | N-Ph | H | H | H | a6 |
| 2-11-040 | N-Ph | N-Ph | t-bu | t-bu | t-bu | a6 |
| 2-11-041 | N-Ph | N-Ph | methyl | methyl | methyl | a6 |
| 2-11-042 | N-Ph | N-Ph | b1 | b1 | b1 | a6 |
| 2-11-043 | N-Ph | N-Ph | b2 | b2 | b2 | a6 |
| 2-11-044 | N-Ph | N-Ph | b3 | b3 | b3 | a6 |
| 2-11-045 | N-Ph | N-Ph | H | H | H | b1 |
| 2-11-046 | N-Ph | N-Ph | t-bu | t-bu | t-bu | b1 |
| 2-11-047 | N-Ph | N-Ph | methyl | methyl | methyl | b1 |
| 2-11-048 | N-Ph | N-Ph | b1 | b1 | b1 | b1 |
| 2-11-049 | N-Ph | N-Ph | b2 | b2 | b2 | b1 |
| 2-11-050 | N-Ph | N-Ph | b3 | b3 | b3 | b1 |
| 2-11-051 | N-Ph | N-Ph | H | H | H | b2 |
| 2-11-052 | N-Ph | N-Ph | t-bu | t-bu | t-bu | b2 |
| 2-11-053 | N-Ph | N-Ph | methyl | methyl | methyl | b2 |
| 2-11-054 | N-Ph | N-Ph | b1 | b1 | b1 | b2 |
| 2-11-055 | N-Ph | N-Ph | b2 | b2 | b2 | b2 |
| 2-11-056 | N-Ph | N-Ph | b3 | b3 | b3 | b2 |
| 2-11-057 | N-Ph | N-Ph | H | H | H | b3 |
| 2-11-058 | N-Ph | N-Ph | t-bu | t-bu | t-bu | b3 |
| 2-11-059 | N-Ph | N-Ph | methyl | methyl | methyl | b3 |
| 2-11-060 | N-Ph | N-Ph | b1 | b1 | b1 | b3 |
| 2-11-061 | N-Ph | N-Ph | b2 | b2 | b2 | b3 |
| 2-11-062 | N-Ph | N-Ph | b3 | b3 | b3 | b3 |
| 2-11-063 | N-Ph | N-Ph | H | H | H | b4 |
| 2-11-064 | N-Ph | N-Ph | t-bu | t-bu | t-bu | b4 |
| 2-11-065 | N-Ph | N-Ph | methyl | methyl | methyl | b4 |
| 2-11-066 | N-Ph | N-Ph | b1 | b1 | b1 | b4 |
| 2-11-067 | N-Ph | N-Ph | b2 | b2 | b2 | b4 |
| 2-11-068 | N-Ph | N-Ph | b3 | b3 | b3 | b4 |
| 2-11-069 | N-Ph | N-Ph | H | H | H | b5 |
| 2-11-070 | N-Ph | N-Ph | t-bu | t-bu | t-bu | b5 |
| 2-11-071 | N-Ph | N-Ph | methyl | methyl | methyl | b5 |
| 2-11-072 | N-Ph | N-Ph | b1 | b1 | b1 | b5 |
| 2-11-073 | N-Ph | N-Ph | b2 | b2 | b2 | b5 |
| 2-11-074 | N-Ph | N-Ph | b3 | b3 | b3 | b5 |
| 2-11-075 | N-Ph | N-Ph | H | H | H | b6 |
| 2-11-076 | N-Ph | N-Ph | t-bu | t-bu | t-bu | b6 |
| 2-11-077 | N-Ph | N-Ph | methyl | methyl | methyl | b6 |
| 2-11-078 | N-Ph | N-Ph | b1 | b1 | b1 | b6 |
| 2-11-079 | N-Ph | N-Ph | b2 | b2 | b2 | b6 |
| 2-11-080 | N-Ph | N-Ph | b3 | b3 | b3 | b6 |
| 2-11-081 | N-Ph | N-Ph | H | H | H | b7 |
| 2-11-082 | N-Ph | N-Ph | t-bu | t-bu | t-bu | b7 |
| 2-11-083 | N-Ph | N-Ph | methyl | methyl | methyl | b7 |
| 2-11-084 | N-Ph | N-Ph | b1 | b1 | b1 | b7 |
| 2-11-085 | N-Ph | N-Ph | b2 | b2 | b2 | b7 |
| 2-11-086 | N-Ph | N-Ph | b3 | b3 | b3 | b7 |
| 2-11-087 | N-Ph | N-Ph | H | H | H | b8 |
| 2-11-088 | N-Ph | N-Ph | t-bu | t-bu | t-bu | b8 |
| 2-11-089 | N-Ph | N-Ph | methyl | methyl | methyl | b8 |
| 2-11-090 | N-Ph | N-Ph | b1 | b1 | b1 | b8 |
| 2-11-091 | N-Ph | N-Ph | b2 | b2 | b2 | b8 |
| 2-11-092 | N-Ph | N-Ph | b3 | b3 | b3 | b8 |
| 2-11-093 | N-Ph | N-Ph | H | H | H | b9 |
| 2-11-094 | N-Ph | N-Ph | t-bu | t-bu | t-bu | b9 |
| 2-11-095 | N-Ph | N-Ph | methyl | methyl | methyl | b9 |
| 2-11-096 | N-Ph | N-Ph | b1 | b1 | b1 | b9 |
| 2-11-097 | N-Ph | N-Ph | b2 | b2 | b2 | b9 |
| 2-11-098 | N-Ph | N-Ph | b3 | b3 | b3 | b9 |
| 2-11-099 | N-Ph | N-Ph | H | H | H | b10 |
| 2-11-100 | N-Ph | N-Ph | t-bu | t-bu | t-bu | b10 |
| 2-11-101 | N-Ph | N-Ph | methyl | methyl | methyl | b10 |
| 2-11-102 | N-Ph | N-Ph | b1 | b1 | b1 | b10 |
| 2-11-103 | N-Ph | N-Ph | b2 | b2 | b2 | b10 |
| 2-11-104 | N-Ph | N-Ph | b3 | b3 | b3 | b10 |
| 2-11-105 | N-tolyl | N-tolyl | H | H | H | H |
| 2-11-106 | N-tolyl | N-tolyl | t-bu | H | t-bu | H |
| 2-11-107 | N-tolyl | N-tolyl | methyl | H | methyl | H |
| 2-11-108 | N-tolyl | N-tolyl | t-bu | t-bu | t-bu | t-bu |
| 2-11-109 | N-tolyl | N-tolyl | methyl | methyl | methyl | methyl |
| 2-11-110 | N-tolyl | N-tolyl | b1 | H | b1 | H |
| 2-11-111 | N-tolyl | N-tolyl | b2 | H | b2 | H |
| 2-11-112 | N-tolyl | N-tolyl | b3 | H | b3 | H |
| 2-11-113 | N-tolyl | N-tolyl | H | H | H | a1 |
| 2-11-114 | N-tolyl | N-tolyl | t-bu | t-bu | t-bu | a1 |
| 2-11-115 | N-tolyl | N-tolyl | methyl | methyl | methyl | a1 |
| 2-11-116 | N-tolyl | N-tolyl | b1 | b1 | b1 | a1 |
| 2-11-117 | N-tolyl | N-tolyl | b2 | b2 | b2 | a1 |
| 2-11-118 | N-tolyl | N-tolyl | b3 | b3 | b3 | a1 |
| 2-11-119 | N-tolyl | N-tolyl | H | H | H | a2 |
| 2-11-120 | N-tolyl | N-tolyl | t-bu | t-bu | t-bu | a2 |
| 2-11-121 | N-tolyl | N-tolyl | methyl | methyl | methyl | a2 |
| 2-11-122 | N-tolyl | N-tolyl | b1 | b1 | b1 | a2 |
| 2-11-123 | N-tolyl | N-tolyl | b2 | b2 | b2 | a2 |
| 2-11-124 | N-tolyl | N-tolyl | b3 | b3 | b3 | a2 |
| 2-11-125 | N-tolyl | N-tolyl | H | H | H | a3 |
| 2-11-126 | N-tolyl | N-tolyl | t-bu | t-bu | t-bu | a3 |
| 2-11-127 | N-tolyl | N-tolyl | methyl | methyl | methyl | a3 |
| 2-11-128 | N-tolyl | N-tolyl | b1 | b1 | b1 | a3 |
| 2-11-129 | N-tolyl | N-tolyl | b2 | b2 | b2 | a3 |
| 2-11-130 | N-tolyl | N-tolyl | b3 | b3 | b3 | a3 |
| 2-11-131 | N-tolyl | N-tolyl | H | H | H | a4 |
| 2-11-132 | N-tolyl | N-tolyl | t-bu | t-bu | t-bu | a4 |
| 2-11-133 | N-tolyl | N-tolyl | methyl | methyl | methyl | a4 |
| 2-11-134 | N-tolyl | N-tolyl | b1 | b1 | b1 | a4 |
| 2-11-135 | N-tolyl | N-tolyl | b2 | b2 | b2 | a4 |
| 2-11-136 | N-tolyl | N-tolyl | b3 | b3 | b3 | a4 |
| 2-11-137 | N-tolyl | N-tolyl | H | H | H | a5 |
| 2-11-138 | N-tolyl | N-tolyl | t-bu | t-bu | t-bu | a5 |
| 2-11-139 | N-tolyl | N-tolyl | methyl | methyl | methyl | a5 |
| 2-11-140 | N-tolyl | N-tolyl | b1 | b1 | b1 | a5 |
| 2-11-141 | N-tolyl | N-tolyl | b2 | b2 | b2 | a5 |
| 2-11-142 | N-tolyl | N-tolyl | b3 | b3 | b3 | a5 |
| 2-11-143 | N-tolyl | N-tolyl | H | H | H | a6 |
| 2-11-144 | N-tolyl | N-tolyl | t-bu | t-bu | t-bu | a6 |
| 2-11-145 | N-tolyl | N-tolyl | methyl | methyl | methyl | a6 |
| 2-11-146 | N-tolyl | N-tolyl | b1 | b1 | b1 | a6 |
| 2-11-147 | N-tolyl | N-tolyl | b2 | b2 | b2 | a6 |
| 2-11-148 | N-tolyl | N-tolyl | b3 | b3 | b3 | a6 |
| 2-11-149 | N-tolyl | N-tolyl | H | H | H | b1 |
| 2-11-150 | N-tolyl | N-tolyl | t-bu | t-bu | t-bu | b1 |
| 2-11-151 | N-tolyl | N-tolyl | methyl | methyl | methyl | b1 |
| 2-11-152 | N-tolyl | N-tolyl | b1 | b1 | b1 | b1 |
| 2-11-153 | N-tolyl | N-tolyl | b2 | b2 | b2 | b1 |
| 2-11-154 | N-tolyl | N-tolyl | b3 | b3 | b3 | b1 |
| 2-11-155 | N-tolyl | N-tolyl | H | H | H | b2 |
| 2-11-156 | N-tolyl | N-tolyl | t-bu | t-bu | t-bu | b2 |
| 2-11-157 | N-tolyl | N-tolyl | methyl | methyl | methyl | b2 |
| 2-11-158 | N-tolyl | N-tolyl | b1 | b1 | b1 | b2 |
| 2-11-159 | N-tolyl | N-tolyl | b2 | b2 | b2 | b2 |
| 2-11-160 | N-tolyl | N-tolyl | b3 | b3 | b3 | b2 |
| 2-11-161 | N-tolyl | N-tolyl | H | H | H | b3 |
| 2-11-162 | N-tolyl | N-tolyl | t-bu | t-bu | t-bu | b3 |
| 2-11-163 | N-tolyl | N-tolyl | methyl | methyl | methyl | b3 |
| 2-11-164 | N-tolyl | N-tolyl | b1 | b1 | b1 | b3 |
| 2-11-165 | N-tolyl | N-tolyl | b2 | b2 | b2 | b3 |
| 2-11-166 | N-tolyl | N-tolyl | b3 | b3 | b3 | b3 |
| 2-11-167 | N-tolyl | N-tolyl | H | H | H | b4 |
| 2-11-168 | N-tolyl | N-tolyl | t-bu | t-bu | t-bu | b4 |
| 2-11-169 | N-tolyl | N-tolyl | methyl | methyl | methyl | b4 |
| 2-11-170 | N-tolyl | N-tolyl | b1 | b1 | b1 | b4 |
| 2-11-171 | N-tolyl | N-tolyl | b2 | b2 | b2 | b4 |
| 2-11-172 | N-tolyl | N-tolyl | b3 | b3 | b3 | b4 |
| 2-11-173 | N-tolyl | N-tolyl | H | H | H | b5 |
| 2-11-174 | N-tolyl | N-tolyl | t-bu | t-bu | t-bu | b5 |
| 2-11-175 | N-tolyl | N-tolyl | methyl | methyl | methyl | b5 |
| 2-11-176 | N-tolyl | N-tolyl | b1 | b1 | b1 | b5 |
| 2-11-177 | N-tolyl | N-tolyl | b2 | b2 | b2 | b5 |
| 2-11-178 | N-tolyl | N-tolyl | b3 | b3 | b3 | b5 |
| 2-11-179 | N-tolyl | N-tolyl | H | H | H | b6 |
| 2-11-180 | N-tolyl | N-tolyl | t-bu | t-bu | t-bu | b6 |
| 2-11-181 | N-tolyl | N-tolyl | methyl | methyl | methyl | b6 |
| 2-11-182 | N-tolyl | N-tolyl | b1 | b1 | b1 | b6 |
| 2-11-183 | N-tolyl | N-tolyl | b2 | b2 | b2 | b6 |
| 2-11-184 | N-tolyl | N-tolyl | b3 | b3 | b3 | b6 |
| 2-11-185 | N-tolyl | N-tolyl | H | H | H | b7 |
| 2-11-186 | N-tolyl | N-tolyl | t-bu | t-bu | t-bu | b7 |
| 2-11-187 | N-tolyl | N-tolyl | methyl | methyl | methyl | b7 |
| 2-11-188 | N-tolyl | N-tolyl | b1 | b1 | b1 | b7 |
| 2-11-189 | N-tolyl | N-tolyl | b2 | b2 | b2 | b7 |
| 2-11-190 | N-tolyl | N-tolyl | b3 | b3 | b3 | b7 |
| 2-11-191 | N-tolyl | N-tolyl | H | H | H | b8 |
| 2-11-192 | N-tolyl | N-tolyl | t-bu | t-bu | t-bu | b8 |
| 2-11-193 | N-tolyl | N-tolyl | methyl | methyl | methyl | b8 |
| 2-11-194 | N-tolyl | N-tolyl | b1 | b1 | b1 | b8 |
| 2-11-195 | N-tolyl | N-tolyl | b2 | b2 | b2 | b8 |
| 2-11-196 | N-tolyl | N-tolyl | b3 | b3 | b3 | b8 |
| 2-11-197 | N-tolyl | N-tolyl | H | H | H | b9 |
| 2-11-198 | N-tolyl | N-tolyl | t-bu | t-bu | t-bu | b9 |
| 2-11-199 | N-tolyl | N-tolyl | methyl | methyl | methyl | b9 |
| 2-11-200 | N-tolyl | N-tolyl | b1 | b1 | b1 | b9 |
| 2-11-201 | N-tolyl | N-tolyl | b2 | b2 | b2 | b9 |
| 2-11-202 | N-tolyl | N-tolyl | b3 | b3 | b3 | b9 |
| 2-11-203 | N-tolyl | N-tolyl | H | H | H | b10 |
| 2-11-204 | N-tolyl | N-tolyl | t-bu | t-bu | t-bu | b10 |
| 2-11-205 | N-tolyl | N-tolyl | methyl | methyl | methyl | b10 |
| 2-11-206 | N-tolyl | N-tolyl | b1 | b1 | b1 | b10 |
| 2-11-207 | N-tolyl | N-tolyl | b2 | b2 | b2 | b10 |
| 2-11-208 | N-tolyl | N-tolyl | b3 | b3 | b3 | b10 |
| 2-11-209 | N-Biph | N-Biph | H | H | H | H |
| 2-11-210 | N-Biph | N-Biph | t-bu | H | t-bu | H |
| 2-11-211 | N-Biph | N-Biph | methyl | H | methyl | H |
| 2-11-212 | N-Biph | N-Biph | t-bu | t-bu | t-bu | t-bu |
| 2-11-213 | N-Biph | N-Biph | methyl | methyl | methyl | methyl |
| 2-11-214 | N-Biph | N-Biph | b1 | H | b1 | H |
| 2-11-215 | N-Biph | N-Biph | b2 | H | b2 | H |
| 2-11-216 | N-Biph | N-Biph | b3 | H | b3 | H |
| 2-11-217 | N-Biph | N-Biph | H | H | H | a1 |
| 2-11-218 | N-Biph | N-Biph | t-bu | t-bu | t-bu | a1 |
| 2-11-219 | N-Biph | N-Biph | methyl | methyl | methyl | a1 |
| 2-11-220 | N-Biph | N-Biph | b1 | b1 | b1 | a1 |
| 2-11-221 | N-Biph | N-Biph | b2 | b2 | b2 | a1 |
| 2-11-222 | N-Biph | N-Biph | b3 | b3 | b3 | a1 |
| 2-11-223 | N-Biph | N-Biph | H | H | H | a2 |
| 2-11-224 | N-Biph | N-Biph | t-bu | t-bu | t-bu | a2 |
| 2-11-225 | N-Biph | N-Biph | methyl | methyl | methyl | a2 |
| 2-11-226 | N-Biph | N-Biph | b1 | b1 | b1 | a2 |
| 2-11-227 | N-Biph | N-Biph | b2 | b2 | b2 | a2 |
| 2-11-228 | N-Biph | N-Biph | b3 | b3 | b3 | a2 |
| 2-11-229 | N-Biph | N-Biph | H | H | H | a3 |
| 2-11-230 | N-Biph | N-Biph | t-bu | t-bu | t-bu | a3 |
| 2-11-231 | N-Biph | N-Biph | methyl | methyl | methyl | a3 |
| 2-11-232 | N-Biph | N-Biph | b1 | b1 | b1 | a3 |
| 2-11-233 | N-Biph | N-Biph | b2 | b2 | b2 | a3 |
| 2-11-234 | N-Biph | N-Biph | b3 | b3 | b3 | a3 |
| 2-11-235 | N-Biph | N-Biph | H | H | H | a4 |
| 2-11-236 | N-Biph | N-Biph | t-bu | t-bu | t-bu | a4 |
| 2-11-237 | N-Biph | N-Biph | methyl | methyl | methyl | a4 |
| 2-11-238 | N-Biph | N-Biph | b1 | b1 | b1 | a4 |
| 2-11-239 | N-Biph | N-Biph | b2 | b2 | b2 | a4 |
| 2-11-240 | N-Biph | N-Biph | b3 | b3 | b3 | a4 |
| 2-11-241 | N-Biph | N-Biph | H | H | H | a5 |
| 2-11-242 | N-Biph | N-Biph | t-bu | t-bu | t-bu | a5 |
| 2-11-243 | N-Biph | N-Biph | methyl | methyl | methyl | a5 |
| 2-11-244 | N-Biph | N-Biph | b1 | b1 | b1 | a5 |
| 2-11-245 | N-Biph | N-Biph | b2 | b2 | b2 | a5 |
| 2-11-246 | N-Biph | N-Biph | b3 | b3 | b3 | a5 |
| 2-11-247 | N-Biph | N-Biph | H | H | H | a6 |
| 2-11-248 | N-Biph | N-Biph | t-bu | t-bu | t-bu | a6 |
| 2-11-249 | N-Biph | N-Biph | methyl | methyl | methyl | a6 |
| 2-11-250 | N-Biph | N-Biph | b1 | b1 | b1 | a6 |
| 2-11-251 | N-Biph | N-Biph | b2 | b2 | b2 | a6 |
| 2-11-252 | N-Biph | N-Biph | b3 | b3 | b3 | a6 |
| 2-11-253 | N-Biph | N-Biph | H | H | H | b1 |
| 2-11-254 | N-Biph | N-Biph | t-bu | t-bu | t-bu | b1 |
| 2-11-255 | N-Biph | N-Biph | methyl | methyl | methyl | b1 |
| 2-11-256 | N-Biph | N-Biph | b1 | b1 | b1 | b1 |
| 2-11-257 | N-Biph | N-Biph | b2 | b2 | b2 | b1 |
| 2-11-258 | N-Biph | N-Biph | b3 | b3 | b3 | b1 |
| 2-11-259 | N-Biph | N-Biph | H | H | H | b2 |
| 2-11-260 | N-Biph | N-Biph | t-bu | t-bu | t-bu | b2 |
| 2-11-261 | N-Biph | N-Biph | methyl | methyl | methyl | b2 |
| 2-11-262 | N-Biph | N-Biph | b1 | b1 | b1 | b2 |
| 2-11-263 | N-Biph | N-Biph | b2 | b2 | b2 | b2 |
| 2-11-264 | N-Biph | N-Biph | b3 | b3 | b3 | b2 |
| 2-11-265 | N-Biph | N-Biph | H | H | H | b3 |
| 2-11-266 | N-Biph | N-Biph | t-bu | t-bu | t-bu | b3 |
| 2-11-267 | N-Biph | N-Biph | methyl | methyl | methyl | b3 |
| 2-11-268 | N-Biph | N-Biph | b1 | b1 | b1 | b3 |
| 2-11-269 | N-Biph | N-Biph | b2 | b2 | b2 | b3 |
| 2-11-270 | N-Biph | N-Biph | b3 | b3 | b3 | b3 |
| 2-11-271 | N-Biph | N-Biph | H | H | H | b4 |
| 2-11-272 | N-Biph | N-Biph | t-bu | t-bu | t-bu | b4 |
| 2-11-273 | N-Biph | N-Biph | methyl | methyl | methyl | b4 |
| 2-11-274 | N-Biph | N-Biph | b1 | b1 | b1 | b4 |
| 2-11-275 | N-Biph | N-Biph | b2 | b2 | b2 | b4 |
| 2-11-276 | N-Biph | N-Biph | b3 | b3 | b3 | b4 |
| 2-11-277 | N-Biph | N-Biph | H | H | H | b5 |
| 2-11-278 | N-Biph | N-Biph | t-bu | t-bu | t-bu | b5 |
| 2-11-279 | N-Biph | N-Biph | methyl | methyl | methyl | b5 |
| 2-11-280 | N-Biph | N-Biph | b1 | b1 | b1 | b5 |
| 2-11-281 | N-Biph | N-Biph | b2 | b2 | b2 | b5 |
| 2-11-282 | N-Biph | N-Biph | b3 | b3 | b3 | b5 |
| 2-11-283 | N-Biph | N-Biph | H | H | H | b6 |
| 2-11-284 | N-Biph | N-Biph | t-bu | t-bu | t-bu | b6 |
| 2-11-285 | N-Biph | N-Biph | methyl | methyl | methyl | b6 |
| 2-11-286 | N-Biph | N-Biph | b1 | b1 | b1 | b6 |
| 2-11-287 | N-Biph | N-Biph | b2 | b2 | b2 | b6 |
| 2-11-288 | N-Biph | N-Biph | b3 | b3 | b3 | b6 |
| 2-11-289 | N-Biph | N-Biph | H | H | H | b7 |
| 2-11-290 | N-Biph | N-Biph | t-bu | t-bu | t-bu | b7 |
| 2-11-291 | N-Biph | N-Biph | methyl | methyl | methyl | b7 |
| 2-11-292 | N-Biph | N-Biph | b1 | b1 | b1 | b7 |
| 2-11-293 | N-Biph | N-Biph | b2 | b2 | b2 | b7 |
| 2-11-294 | N-Biph | N-Biph | b3 | b3 | b3 | b7 |
| 2-11-295 | N-Biph | N-Biph | H | H | H | b8 |
| 2-11-296 | N-Biph | N-Biph | t-bu | t-bu | t-bu | b8 |
| 2-11-297 | N-Biph | N-Biph | methyl | methyl | methyl | b8 |
| 2-11-298 | N-Biph | N-Biph | b1 | b1 | b1 | b8 |
| 2-11-299 | N-Biph | N-Biph | b2 | b2 | b2 | b8 |
| 2-11-300 | N-Biph | N-Biph | b3 | b3 | b3 | b8 |
| 2-11-301 | N-Biph | N-Biph | H | H | H | b9 |
| 2-11-302 | N-Biph | N-Biph | t-bu | t-bu | t-bu | b9 |
| 2-11-303 | N-Biph | N-Biph | methyl | methyl | methyl | b9 |
| 2-11-304 | N-Biph | N-Biph | b1 | b1 | b1 | b9 |
| 2-11-305 | N-Biph | N-Biph | b2 | b2 | b2 | b9 |
| 2-11-306 | N-Biph | N-Biph | b3 | b3 | b3 | b9 |
| 2-11-307 | N-Biph | N-Biph | H | H | H | b10 |
| 2-11-308 | N-Biph | N-Biph | t-bu | t-bu | t-bu | b10 |
| 2-11-309 | N-Biph | N-Biph | methyl | methyl | methyl | b10 |
| 2-11-310 | N-Biph | N-Biph | b1 | b1 | b1 | b10 |
| 2-11-311 | N-Biph | N-Biph | b2 | b2 | b2 | b10 |
| 2-11-312 | N-Biph | N-Biph | b3 | b3 | b3 | b10 |
| 2-11-313 | O | N-Ph | H | H | H | H |
| 2-11-314 | O | N-Ph | t-bu | H | t-bu | H |
| 2-11-315 | O | N-Ph | methyl | H | methyl | H |
| 2-11-316 | O | N-Ph | t-bu | t-bu | t-bu | t-bu |
| 2-11-317 | O | N-Ph | methyl | methyl | methyl | methyl |
| 2-11-318 | O | N-Ph | b1 | H | b1 | H |
| 2-11-319 | O | N-Ph | b2 | H | b2 | H |
| 2-11-320 | O | N-Ph | b3 | H | b3 | H |
| 2-11-321 | O | N-Ph | H | H | H | a1 |
| 2-11-322 | O | N-Ph | t-bu | t-bu | t-bu | a1 |
| 2-11-323 | O | N-Ph | methyl | methyl | methyl | a1 |
| 2-11-324 | O | N-Ph | b1 | b1 | b1 | a1 |
| 2-11-325 | O | N-Ph | b2 | b2 | b2 | a1 |
| 2-11-326 | O | N-Ph | b3 | b3 | b3 | a1 |
| 2-11-327 | O | N-Ph | H | H | H | a2 |
| 2-11-328 | O | N-Ph | t-bu | t-bu | t-bu | a2 |
| 2-11-329 | O | N-Ph | methyl | methyl | methyl | a2 |
| 2-11-330 | O | N-Ph | b1 | b1 | b1 | a2 |
| 2-11-331 | O | N-Ph | b2 | b2 | b2 | a2 |
| 2-11-332 | O | N-Ph | b3 | b3 | b3 | a2 |
| 2-11-333 | O | N-Ph | H | H | H | a3 |
| 2-11-334 | O | N-Ph | t-bu | t-bu | t-bu | a3 |
| 2-11-335 | O | N-Ph | methyl | methyl | methyl | a3 |
| 2-11-336 | O | N-Ph | b1 | b1 | b1 | a3 |
| 2-11-337 | O | N-Ph | b2 | b2 | b2 | a3 |
| 2-11-338 | O | N-Ph | b3 | b3 | b3 | a3 |
| 2-11-339 | O | N-Ph | H | H | H | a4 |
| 2-11-340 | O | N-Ph | t-bu | t-bu | t-bu | a4 |
| 2-11-341 | O | N-Ph | methyl | methyl | methyl | a4 |
| 2-11-342 | O | N-Ph | b1 | b1 | b1 | a4 |
| 2-11-343 | O | N-Ph | b2 | b2 | b2 | a4 |
| 2-11-344 | O | N-Ph | b3 | b3 | b3 | a4 |
| 2-11-345 | O | N-Ph | H | H | H | a5 |
| 2-11-346 | O | N-Ph | t-bu | t-bu | t-bu | a5 |
| 2-11-347 | O | N-Ph | methyl | methyl | methyl | a5 |
| 2-11-348 | O | N-Ph | b1 | b1 | b1 | a5 |
| 2-11-349 | O | N-Ph | b2 | b2 | b2 | a5 |
| 2-11-350 | O | N-Ph | b3 | b3 | b3 | a5 |
| 2-11-351 | O | N-Ph | H | H | H | a6 |
| 2-11-352 | O | N-Ph | t-bu | t-bu | t-bu | a6 |
| 2-11-353 | O | N-Ph | methyl | methyl | methyl | a6 |
| 2-11-354 | O | N-Ph | b1 | b1 | b1 | a6 |
| 2-11-355 | O | N-Ph | b2 | b2 | b2 | a6 |
| 2-11-356 | O | N-Ph | b3 | b3 | b3 | a6 |
| 2-11-357 | O | N-Ph | H | H | H | b1 |
| 2-11-358 | O | N-Ph | t-bu | t-bu | t-bu | b1 |
| 2-11-359 | O | N-Ph | methyl | methyl | methyl | b1 |
| 2-11-360 | O | N-Ph | b1 | b1 | b1 | b1 |
| 2-11-361 | O | N-Ph | b2 | b2 | b2 | b1 |
| 2-11-362 | O | N-Ph | b3 | b3 | b3 | b1 |
| 2-11-363 | O | N-Ph | H | H | H | b2 |
| 2-11-364 | O | N-Ph | t-bu | t-bu | t-bu | b2 |
| 2-11-365 | O | N-Ph | methyl | methyl | methyl | b2 |
| 2-11-366 | O | N-Ph | b1 | b1 | b1 | b2 |
| 2-11-367 | O | N-Ph | b2 | b2 | b2 | b2 |
| 2-11-368 | O | N-Ph | b3 | b3 | b3 | b2 |
| 2-11-369 | O | N-Ph | H | H | H | b3 |
| 2-11-370 | O | N-Ph | t-bu | t-bu | t-bu | b3 |
| 2-11-371 | O | N-Ph | methyl | methyl | methyl | b3 |
| 2-11-372 | O | N-Ph | b1 | b1 | b1 | b3 |
| 2-11-373 | O | N-Ph | b2 | b2 | b2 | b3 |
| 2-11-374 | O | N-Ph | b3 | b3 | b3 | b3 |
| 2-11-375 | O | N-Ph | H | H | H | b4 |
| 2-11-376 | O | N-Ph | t-bu | t-bu | t-bu | b4 |
| 2-11-377 | O | N-Ph | methyl | methyl | methyl | b4 |
| 2-11-378 | O | N-Ph | b1 | b1 | b1 | b4 |
| 2-11-379 | O | N-Ph | b2 | b2 | b2 | b4 |
| 2-11-380 | O | N-Ph | b3 | b3 | b3 | b4 |
| 2-11-381 | O | N-Ph | H | H | H | b5 |
| 2-11-382 | O | N-Ph | t-bu | t-bu | t-bu | b5 |
| 2-11-383 | O | N-Ph | methyl | methyl | methyl | b5 |
| 2-11-384 | O | N-Ph | b1 | b1 | b1 | b5 |
| 2-11-385 | O | N-Ph | b2 | b2 | b2 | b5 |
| 2-11-386 | O | N-Ph | b3 | b3 | b3 | b5 |
| 2-11-387 | O | N-Ph | H | H | H | b6 |
| 2-11-388 | O | N-Ph | t-bu | t-bu | t-bu | b6 |
| 2-11-389 | O | N-Ph | methyl | methyl | methyl | b6 |
| 2-11-390 | O | N-Ph | b1 | b1 | b1 | b6 |
| 2-11-391 | O | N-Ph | b2 | b2 | b2 | b6 |
| 2-11-392 | O | N-Ph | b3 | b3 | b3 | b6 |
| 2-11-393 | O | N-Ph | H | H | H | b7 |
| 2-11-394 | O | N-Ph | t-bu | t-bu | t-bu | b7 |
| 2-11-395 | O | N-Ph | methyl | methyl | methyl | b7 |
| 2-11-396 | O | N-Ph | b1 | b1 | b1 | b7 |
| 2-11-397 | O | N-Ph | b2 | b2 | b2 | b7 |
| 2-11-398 | O | N-Ph | b3 | b3 | b3 | b7 |
| 2-11-399 | O | N-Ph | H | H | H | b8 |
| 2-11-400 | O | N-Ph | t-bu | t-bu | t-bu | b8 |
| 2-11-401 | O | N-Ph | methyl | methyl | methyl | b8 |
| 2-11-402 | O | N-Ph | b1 | b1 | b1 | b8 |
| 2-11-403 | O | N-Ph | b2 | b2 | b2 | b8 |
| 2-11-404 | O | N-Ph | b3 | b3 | b3 | b8 |
| 2-11-405 | O | N-Ph | H | H | H | b9 |
| 2-11-406 | O | N-Ph | t-bu | t-bu | t-bu | b9 |
| 2-11-407 | O | N-Ph | methyl | methyl | methyl | b9 |
| 2-11-408 | O | N-Ph | b1 | b1 | b1 | b9 |
| 2-11-409 | O | N-Ph | b2 | b2 | b2 | b9 |
| 2-11-410 | O | N-Ph | b3 | b3 | b3 | b9 |
| 2-11-411 | O | N-Ph | H | H | H | b10 |
| 2-11-412 | O | N-Ph | t-bu | t-bu | t-bu | b10 |
| 2-11-413 | O | N-Ph | methyl | methyl | methyl | b10 |
| 2-11-414 | O | N-Ph | b1 | b1 | b1 | b10 |
| 2-11-415 | O | N-Ph | b2 | b2 | b2 | b10 |
| 2-11-416 | O | N-Ph | b3 | b3 | b3 | b10 |
| 2-11-417 | S | N-Ph | H | H | H | H |
| 2-11-418 | S | N-Ph | t-bu | H | t-bu | H |
| 2-11-419 | S | N-Ph | methyl | H | methyl | H |
| 2-11-420 | S | N-Ph | t-bu | t-bu | t-bu | t-bu |
| 2-11-421 | S | N-Ph | methyl | methyl | methyl | methyl |
| 2-11-422 | S | N-Ph | b1 | H | b1 | H |
| 2-11-423 | S | N-Ph | b2 | H | b2 | H |
| 2-11-424 | S | N-Ph | b3 | H | b3 | H |
| 2-11-425 | S | N-Ph | H | H | H | a1 |
| 2-11-426 | S | N-Ph | t-bu | t-bu | t-bu | a1 |
| 2-11-427 | S | N-Ph | methyl | methyl | methyl | a1 |
| 2-11-428 | S | N-Ph | b1 | b1 | b1 | a1 |
| 2-11-429 | S | N-Ph | b2 | b2 | b2 | a1 |
| 2-11-430 | S | N-Ph | b3 | b3 | b3 | a1 |
| 2-11-431 | S | N-Ph | H | H | H | a2 |
| 2-11-432 | S | N-Ph | t-bu | t-bu | t-bu | a2 |
| 2-11-433 | S | N-Ph | methyl | methyl | methyl | a2 |
| 2-11-434 | S | N-Ph | b1 | b1 | b1 | a2 |
| 2-11-435 | S | N-Ph | b2 | b2 | b2 | a2 |
| 2-11-436 | S | N-Ph | b3 | b3 | b3 | a2 |
| 2-11-437 | S | N-Ph | H | H | H | a3 |
| 2-11-438 | S | N-Ph | t-bu | t-bu | t-bu | a3 |
| 2-11-439 | S | N-Ph | methyl | methyl | methyl | a3 |
| 2-11-440 | S | N-Ph | b1 | b1 | b1 | a3 |
| 2-11-441 | S | N-Ph | b2 | b2 | b2 | a3 |
| 2-11-442 | S | N-Ph | b3 | b3 | b3 | a3 |
| 2-11-443 | S | N-Ph | H | H | H | a4 |
| 2-11-444 | S | N-Ph | t-bu | t-bu | t-bu | a4 |
| 2-11-445 | S | N-Ph | methyl | methyl | methyl | a4 |
| 2-11-446 | S | N-Ph | b1 | b1 | b1 | a4 |
| 2-11-447 | S | N-Ph | b2 | b2 | b2 | a4 |
| 2-11-448 | S | N-Ph | b3 | b3 | b3 | a4 |
| 2-11-449 | S | N-Ph | H | H | H | a5 |
| 2-11-450 | S | N-Ph | t-bu | t-bu | t-bu | a5 |
| 2-11-451 | S | N-Ph | methyl | methyl | methyl | a5 |
| 2-11-452 | S | N-Ph | b1 | b1 | b1 | a5 |
| 2-11-453 | S | N-Ph | b2 | b2 | b2 | a5 |
| 2-11-454 | S | N-Ph | b3 | b3 | b3 | a5 |
| 2-11-455 | S | N-Ph | H | H | H | a6 |
| 2-11-456 | S | N-Ph | t-bu | t-bu | t-bu | a6 |
| 2-11-457 | S | N-Ph | methyl | methyl | methyl | a6 |
| 2-11-458 | S | N-Ph | b1 | b1 | b1 | a6 |
| 2-11-459 | S | N-Ph | b2 | b2 | b2 | a6 |
| 2-11-460 | S | N-Ph | b3 | b3 | b3 | a6 |
| 2-11-461 | S | N-Ph | H | H | H | b1 |
| 2-11-462 | S | N-Ph | t-bu | t-bu | t-bu | b1 |
| 2-11-463 | S | N-Ph | methyl | methyl | methyl | b1 |
| 2-11-464 | S | N-Ph | b1 | b1 | b1 | b1 |
| 2-11-465 | S | N-Ph | b2 | b2 | b2 | b1 |
| 2-11-466 | S | N-Ph | b3 | b3 | b3 | b1 |
| 2-11-467 | S | N-Ph | H | H | H | b2 |
| 2-11-468 | S | N-Ph | t-bu | t-bu | t-bu | b2 |
| 2-11-469 | S | N-Ph | methyl | methyl | methyl | b2 |
| 2-11-470 | S | N-Ph | b1 | b1 | b1 | b2 |
| 2-11-471 | S | N-Ph | b2 | b2 | b2 | b2 |
| 2-11-472 | S | N-Ph | b3 | b3 | b3 | b2 |
| 2-11-473 | S | N-Ph | H | H | H | b3 |
| 2-11-474 | S | N-Ph | t-bu | t-bu | t-bu | b3 |
| 2-11-475 | S | N-Ph | methyl | methyl | methyl | b3 |
| 2-11-476 | S | N-Ph | b1 | b1 | b1 | b3 |
| 2-11-477 | S | N-Ph | b2 | b2 | b2 | b3 |
| 2-11-478 | S | N-Ph | b3 | b3 | b3 | b3 |
| 2-11-479 | S | N-Ph | H | H | H | b4 |
| 2-11-480 | S | N-Ph | t-bu | t-bu | t-bu | b4 |
| 2-11-481 | S | N-Ph | methyl | methyl | methyl | b4 |
| 2-11-482 | S | N-Ph | b1 | b1 | b1 | b4 |
| 2-11-483 | S | N-Ph | b2 | b2 | b2 | b4 |
| 2-11-484 | S | N-Ph | b3 | b3 | b3 | b4 |
| 2-11-485 | S | N-Ph | H | H | H | b5 |
| 2-11-486 | S | N-Ph | t-bu | t-bu | t-bu | b5 |
| 2-11-487 | S | N-Ph | methyl | methyl | methyl | b5 |
| 2-11-488 | S | N-Ph | b1 | b1 | b1 | b5 |
| 2-11-489 | S | N-Ph | b2 | b2 | b2 | b5 |
| 2-11-490 | S | N-Ph | b3 | b3 | b3 | b5 |
| 2-11-491 | S | N-Ph | H | H | H | b6 |
| 2-11-492 | S | N-Ph | t-bu | t-bu | t-bu | b6 |
| 2-11-493 | S | N-Ph | methyl | methyl | methyl | b6 |
| 2-11-494 | S | N-Ph | b1 | b1 | b1 | b6 |
| 2-11-495 | S | N-Ph | b2 | b2 | b2 | b6 |
| 2-11-496 | S | N-Ph | b3 | b3 | b3 | b6 |
| 2-11-497 | S | N-Ph | H | H | H | b7 |
| 2-11-498 | S | N-Ph | t-bu | t-bu | t-bu | b7 |
| 2-11-499 | S | N-Ph | methyl | methyl | methyl | b7 |
| 2-11-500 | S | N-Ph | b1 | b1 | b1 | b7 |
| 2-11-501 | S | N-Ph | b2 | b2 | b2 | b7 |
| 2-11-502 | S | N-Ph | b3 | b3 | b3 | b7 |
| 2-11-503 | S | N-Ph | H | H | H | b8 |
| 2-11-504 | S | N-Ph | t-bu | t-bu | t-bu | b8 |
| 2-11-505 | S | N-Ph | methyl | methyl | methyl | b8 |
| 2-11-506 | S | N-Ph | b1 | b1 | b1 | b8 |
| 2-11-507 | S | N-Ph | b2 | b2 | b2 | b8 |
| 2-11-508 | S | N-Ph | b3 | b3 | b3 | b8 |
| 2-11-509 | S | N-Ph | H | H | H | b9 |
| 2-11-510 | S | N-Ph | t-bu | t-bu | t-bu | b9 |
| 2-11-511 | S | N-Ph | methyl | methyl | methyl | b9 |
| 2-11-512 | S | N-Ph | b1 | b1 | b1 | b9 |
| 2-11-513 | S | N-Ph | b2 | b2 | b2 | b9 |
| 2-11-514 | S | N-Ph | b3 | b3 | b3 | b9 |
| 2-11-515 | S | N-Ph | H | H | H | b10 |
| 2-11-516 | S | N-Ph | t-bu | t-bu | t-bu | b10 |
| 2-11-517 | S | N-Ph | methyl | methyl | methyl | b10 |
| 2-11-518 | S | N-Ph | b1 | b1 | b1 | b10 |
| 2-11-519 | S | N-Ph | b2 | b2 | b2 | b10 |
| 2-11-520 | S | N-Ph | b3 | b3 | b3 | b10 |

Group 2-12
The compounds are represented by the formula 2-12 below, wherein CY₁ is a benzene group, X₁ is B, and Y1, Y2, R2b, R2d, R3a, (R1)a1 are each shown in the table 12 below, 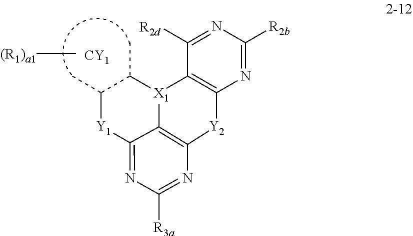
TABLE 12

| Compound | | | | | | |
|---|---|---|---|---|---|---|
| No. | Y1 | Y2 | R2b | R2d | R3a | (R1)a1 |
| 2-12-001 | N-Ph | N-Ph | H | H | H | H |
| 2-12-002 | N-Ph | N-Ph | t-bu | H | t-bu | H |
| 2-12-003 | N-Ph | N-Ph | methyl | H | methyl | H |
| 2-12-004 | N-Ph | N-Ph | t-bu | t-bu | t-bu | t-bu |
| 2-12-005 | N-Ph | N-Ph | methyl | methyl | methyl | methyl |
| 2-12-006 | N-Ph | N-Ph | b1 | H | b1 | H |
| 2-12-007 | N-Ph | N-Ph | b2 | H | b2 | H |
| 2-12-008 | N-Ph | N-Ph | b3 | H | b3 | H |
| 2-12-009 | N-Ph | N-Ph | H | H | H | a1 |
| 2-12-010 | N-Ph | N-Ph | t-bu | H | t-bu | a1 |
| 2-12-011 | N-Ph | N-Ph | methyl | H | methyl | a1 |
| 2-12-012 | N-Ph | N-Ph | b1 | H | b1 | a1 |
| 2-12-013 | N-Ph | N-Ph | b2 | H | b2 | a1 |
| 2-12-014 | N-Ph | N-Ph | b3 | H | b3 | a1 |
| 2-12-015 | N-Ph | N-Ph | H | H | H | a2 |
| 2-12-016 | N-Ph | N-Ph | t-bu | H | t-bu | a2 |
| 2-12-017 | N-Ph | N-Ph | methyl | H | methyl | a2 |
| 2-12-018 | N-Ph | N-Ph | b1 | H | b1 | a2 |
| 2-12-019 | N-Ph | N-Ph | b2 | H | b2 | a2 |
| 2-12-020 | N-Ph | N-Ph | b3 | H | b3 | a2 |
| 2-12-021 | N-Ph | N-Ph | H | H | H | a3 |
| 2-12-022 | N-Ph | N-Ph | t-bu | H | t-bu | a3 |
| 2-12-023 | N-Ph | N-Ph | methyl | H | methyl | a3 |
| 2-12-024 | N-Ph | N-Ph | b1 | H | b1 | a3 |
| 2-12-025 | N-Ph | N-Ph | b2 | H | b2 | a3 |
| 2-12-026 | N-Ph | N-Ph | b3 | H | b3 | a3 |
| 2-12-027 | N-Ph | N-Ph | H | H | H | a4 |
| 2-12-028 | N-Ph | N-Ph | t-bu | H | t-bu | a4 |
| 2-12-029 | N-Ph | N-Ph | methyl | H | methyl | a4 |
| 2-12-030 | N-Ph | N-Ph | b1 | H | b1 | a4 |
| 2-12-031 | N-Ph | N-Ph | b2 | H | b2 | a4 |
| 2-12-032 | N-Ph | N-Ph | b3 | H | b3 | a4 |
| 2-12-033 | N-Ph | N-Ph | H | H | H | a5 |
| 2-12-034 | N-Ph | N-Ph | t-bu | H | t-bu | a5 |
| 2-12-035 | N-Ph | N-Ph | methyl | H | methyl | a5 |
| 2-12-036 | N-Ph | N-Ph | b1 | H | b1 | a5 |
| 2-12-037 | N-Ph | N-Ph | b2 | H | b2 | a5 |
| 2-12-038 | N-Ph | N-Ph | b3 | H | b3 | a5 |
| 2-12-039 | N-Ph | N-Ph | H | H | H | a6 |
| 2-12-040 | N-Ph | N-Ph | t-bu | H | t-bu | a6 |
| 2-12-041 | N-Ph | N-Ph | methyl | H | methyl | a6 |
| 2-12-042 | N-Ph | N-Ph | b1 | H | b1 | a6 |
| 2-12-043 | N-Ph | N-Ph | b2 | H | b2 | a6 |
| 2-12-044 | N-Ph | N-Ph | b3 | H | b3 | a6 |
| 2-12-045 | N-Ph | N-Ph | H | H | H | b1 |
| 2-12-046 | N-Ph | N-Ph | t-bu | H | t-bu | b1 |
| 2-12-047 | N-Ph | N-Ph | methyl | H | methyl | b1 |
| 2-12-048 | N-Ph | N-Ph | b1 | H | b1 | b1 |
| 2-12-049 | N-Ph | N-Ph | b2 | H | b2 | b1 |
| 2-12-050 | N-Ph | N-Ph | b3 | H | b3 | b1 |
| 2-12-051 | N-Ph | N-Ph | H | H | H | b2 |
| 2-12-052 | N-Ph | N-Ph | t-bu | H | t-bu | b2 |
| 2-12-053 | N-Ph | N-Ph | methyl | H | methyl | b2 |
| 2-12-054 | N-Ph | N-Ph | b1 | H | b1 | b2 |
| 2-12-055 | N-Ph | N-Ph | b2 | H | b2 | b2 |
| 2-12-056 | N-Ph | N-Ph | b3 | H | b3 | b2 |
| 2-12-057 | N-Ph | N-Ph | H | H | H | b3 |
| 2-12-058 | N-Ph | N-Ph | t-bu | H | t-bu | b3 |
| 2-12-059 | N-Ph | N-Ph | methyl | H | methyl | b3 |
| 2-12-060 | N-Ph | N-Ph | b1 | H | b1 | b3 |
| 2-12-061 | N-Ph | N-Ph | b2 | H | b2 | b3 |
| 2-12-062 | N-Ph | N-Ph | b3 | H | b3 | b3 |
| 2-12-063 | N-Ph | N-Ph | H | H | H | b4 |
| 2-12-064 | N-Ph | N-Ph | t-bu | H | t-bu | b4 |
| 2-12-065 | N-Ph | N-Ph | methyl | H | methyl | b4 |
| 2-12-066 | N-Ph | N-Ph | b1 | H | b1 | b4 |
| 2-12-067 | N-Ph | N-Ph | b2 | H | b2 | b4 |
| 2-12-068 | N-Ph | N-Ph | b3 | H | b3 | b4 |
| 2-12-069 | N-Ph | N-Ph | H | H | H | b5 |
| 2-12-070 | N-Ph | N-Ph | t-bu | H | t-bu | b5 |
| 2-12-071 | N-Ph | N-Ph | methyl | H | methyl | b5 |
| 2-12-072 | N-Ph | N-Ph | b1 | H | b1 | b5 |
| 2-12-073 | N-Ph | N-Ph | b2 | H | b2 | b5 |
| 2-12-074 | N-Ph | N-Ph | b3 | H | b3 | b5 |
| 2-12-075 | N-Ph | N-Ph | H | H | H | b6 |
| 2-12-076 | N-Ph | N-Ph | t-bu | H | t-bu | b6 |
| 2-12-077 | N-Ph | N-Ph | methyl | H | methyl | b6 |
| 2-12-078 | N-Ph | N-Ph | b1 | H | b1 | b6 |
| 2-12-079 | N-Ph | N-Ph | b2 | H | b2 | b6 |
| 2-12-080 | N-Ph | N-Ph | b3 | H | b3 | b6 |
| 2-12-081 | N-Ph | N-Ph | H | H | H | b7 |
| 2-12-082 | N-Ph | N-Ph | t-bu | H | t-bu | b7 |
| 2-12-083 | N-Ph | N-Ph | methyl | H | methyl | b7 |
| 2-12-084 | N-Ph | N-Ph | b1 | H | b1 | b7 |
| 2-12-085 | N-Ph | N-Ph | b2 | H | b2 | b7 |
| 2-12-086 | N-Ph | N-Ph | b3 | H | b3 | b7 |
| 2-12-087 | N-Ph | N-Ph | H | H | H | b8 |
| 2-12-088 | N-Ph | N-Ph | t-bu | H | t-bu | b8 |
| 2-12-089 | N-Ph | N-Ph | methyl | H | methyl | b8 |
| 2-12-090 | N-Ph | N-Ph | b1 | H | b1 | b8 |
| 2-12-091 | N-Ph | N-Ph | b2 | H | b2 | b8 |
| 2-12-092 | N-Ph | N-Ph | b3 | H | b3 | b8 |
| 2-12-093 | N-Ph | N-Ph | H | H | H | b9 |
| 2-12-094 | N-Ph | N-Ph | t-bu | H | t-bu | b9 |
| 2-12-095 | N-Ph | N-Ph | methyl | H | methyl | b9 |
| 2-12-096 | N-Ph | N-Ph | b1 | H | b1 | b9 |
| 2-12-097 | N-Ph | N-Ph | b2 | H | b2 | b9 |
| 2-12-098 | N-Ph | N-Ph | b3 | H | b3 | b9 |
| 2-12-099 | N-Ph | N-Ph | H | H | H | b10 |
| 2-12-100 | N-Ph | N-Ph | t-bu | H | t-bu | b10 |
| 2-12-101 | N-Ph | N-Ph | methyl | H | methyl | b10 |
| 2-12-102 | N-Ph | N-Ph | b1 | H | b1 | b10 |
| 2-12-103 | N-Ph | N-Ph | b2 | H | b2 | b10 |
| 2-12-104 | N-Ph | N-Ph | b3 | H | b3 | b10 |
| 2-12-105 | N-tolyl | N-tolyl | H | H | H | H |
| 2-12-106 | N-tolyl | N-tolyl | t-bu | H | t-bu | H |
| 2-12-107 | N-tolyl | N-tolyl | methyl | H | methyl | H |
| 2-12-108 | N-tolyl | N-tolyl | t-bu | t-bu | t-bu | t-bu |
| 2-12-109 | N-tolyl | N-tolyl | methyl | methyl | methyl | methyl |
| 2-12-110 | N-tolyl | N-tolyl | b1 | H | b1 | H |
| 2-12-111 | N-tolyl | N-tolyl | b2 | H | b2 | H |
| 2-12-112 | N-tolyl | N-tolyl | b3 | H | b3 | H |
| 2-12-113 | N-tolyl | N-tolyl | H | H | H | a1 |
| 2-12-114 | N-tolyl | N-tolyl | t-bu | H | t-bu | a1 |
| 2-12-115 | N-tolyl | N-tolyl | methyl | H | methyl | a1 |
| 2-12-116 | N-tolyl | N-tolyl | b1 | H | b1 | a1 |
| 2-12-117 | N-tolyl | N-tolyl | b2 | H | b2 | a1 |
| 2-12-118 | N-tolyl | N-tolyl | b3 | H | b3 | a1 |
| 2-12-119 | N-tolyl | N-tolyl | H | H | H | a2 |
| 2-12-120 | N-tolyl | N-tolyl | t-bu | H | t-bu | a2 |
| 2-12-121 | N-tolyl | N-tolyl | methyl | H | methyl | a2 |
| 2-12-122 | N-tolyl | N-tolyl | b1 | H | b1 | a2 |
| 2-12-123 | N-tolyl | N-tolyl | b2 | H | b2 | a2 |
| 2-12-124 | N-tolyl | N-tolyl | b3 | H | b3 | a2 |
| 2-12-125 | N-tolyl | N-tolyl | H | H | H | a3 |
| 2-12-126 | N-tolyl | N-tolyl | t-bu | H | t-bu | a3 |
| 2-12-127 | N-tolyl | N-tolyl | methyl | H | methyl | a3 |
| 2-12-128 | N-tolyl | N-tolyl | b1 | H | b1 | a3 |
| 2-12-129 | N-tolyl | N-tolyl | b2 | H | b2 | a3 |
| 2-12-130 | N-tolyl | N-tolyl | b3 | H | b3 | a3 |
| 2-12-131 | N-tolyl | N-tolyl | H | H | H | a4 |
| 2-12-132 | N-tolyl | N-tolyl | t-bu | H | t-bu | a4 |
| 2-12-133 | N-tolyl | N-tolyl | methyl | H | methyl | a4 |
| 2-12-134 | N-tolyl | N-tolyl | b1 | H | b1 | a4 |
| 2-12-135 | N-tolyl | N-tolyl | b2 | H | b2 | a4 |
| 2-12-136 | N-tolyl | N-tolyl | b3 | H | b3 | a4 |
| 2-12-137 | N-tolyl | N-tolyl | H | H | H | a5 |
| 2-12-138 | N-tolyl | N-tolyl | t-bu | H | t-bu | a5 |
| 2-12-139 | N-tolyl | N-tolyl | methyl | H | methyl | a5 |
| 2-12-140 | N-tolyl | N-tolyl | b1 | H | b1 | a5 |
| 2-12-141 | N-tolyl | N-tolyl | b2 | H | b2 | a5 |
| 2-12-142 | N-tolyl | N-tolyl | b3 | H | b3 | a5 |
| 2-12-143 | N-tolyl | N-tolyl | H | H | H | a6 |
| 2-12-144 | N-tolyl | N-tolyl | t-bu | H | t-bu | a6 |
| 2-12-145 | N-tolyl | N-tolyl | methyl | H | methyl | a6 |
| 2-12-146 | N-tolyl | N-tolyl | b1 | H | b1 | a6 |
| 2-12-147 | N-tolyl | N-tolyl | b2 | H | b2 | a6 |
| 2-12-148 | N-tolyl | N-tolyl | b3 | H | b3 | a6 |
| 2-12-149 | N-tolyl | N-tolyl | H | H | H | b1 |
| 2-12-150 | N-tolyl | N-tolyl | t-bu | H | t-bu | b1 |
| 2-12-151 | N-tolyl | N-tolyl | methyl | H | methyl | b1 |
| 2-12-152 | N-tolyl | N-tolyl | b1 | H | b1 | b1 |
| 2-12-153 | N-tolyl | N-tolyl | b2 | H | b2 | b1 |
| 2-12-154 | N-tolyl | N-tolyl | b3 | H | b3 | b1 |
| 2-12-155 | N-tolyl | N-tolyl | H | H | H | b2 |
| 2-12-156 | N-tolyl | N-tolyl | t-bu | H | t-bu | b2 |
| 2-12-157 | N-tolyl | N-tolyl | methyl | H | methyl | b2 |
| 2-12-158 | N-tolyl | N-tolyl | b1 | H | b1 | b2 |
| 2-12-159 | N-tolyl | N-tolyl | b2 | H | b2 | b2 |
| 2-12-160 | N-tolyl | N-tolyl | b3 | H | b3 | b2 |
| 2-12-161 | N-tolyl | N-tolyl | H | H | H | b3 |
| 2-12-162 | N-tolyl | N-tolyl | t-bu | H | t-bu | b3 |
| 2-12-163 | N-tolyl | N-tolyl | methyl | H | methyl | b3 |
| 2-12-164 | N-tolyl | N-tolyl | b1 | H | b1 | b3 |
| 2-12-165 | N-tolyl | N-tolyl | b2 | H | b2 | b3 |
| 2-12-166 | N-tolyl | N-tolyl | b3 | H | b3 | b3 |
| 2-12-167 | N-tolyl | N-tolyl | H | H | H | b4 |
| 2-12-168 | N-tolyl | N-tolyl | t-bu | H | t-bu | b4 |
| 2-12-169 | N-tolyl | N-tolyl | methyl | H | methyl | b4 |
| 2-12-170 | N-tolyl | N-tolyl | b1 | H | b1 | b4 |
| 2-12-171 | N-tolyl | N-tolyl | b2 | H | b2 | b4 |
| 2-12-172 | N-tolyl | N-tolyl | b3 | H | b3 | b4 |
| 2-12-173 | N-tolyl | N-tolyl | H | H | H | b5 |
| 2-12-174 | N-tolyl | N-tolyl | t-bu | H | t-bu | b5 |
| 2-12-175 | N-tolyl | N-tolyl | methyl | H | methyl | b5 |
| 2-12-176 | N-tolyl | N-tolyl | b1 | H | b1 | b5 |
| 2-12-177 | N-tolyl | N-tolyl | b2 | H | b2 | b5 |
| 2-12-178 | N-tolyl | N-tolyl | b3 | H | b3 | b5 |
| 2-12-179 | N-tolyl | N-tolyl | H | H | H | b6 |
| 2-12-180 | N-tolyl | N-tolyl | t-bu | H | t-bu | b6 |
| 2-12-181 | N-tolyl | N-tolyl | methyl | H | methyl | b6 |
| 2-12-182 | N-tolyl | N-tolyl | b1 | H | b1 | b6 |
| 2-12-183 | N-tolyl | N-tolyl | b2 | H | b2 | b6 |
| 2-12-184 | N-tolyl | N-tolyl | b3 | H | b3 | b6 |
| 2-12-185 | N-tolyl | N-tolyl | H | H | H | b7 |
| 2-12-186 | N-tolyl | N-tolyl | t-bu | H | t-bu | b7 |
| 2-12-187 | N-tolyl | N-tolyl | methyl | H | methyl | b7 |
| 2-12-188 | N-tolyl | N-tolyl | b1 | H | b1 | b7 |
| 2-12-189 | N-tolyl | N-tolyl | b2 | H | b2 | b7 |
| 2-12-190 | N-tolyl | N-tolyl | b3 | H | b3 | b7 |
| 2-12-191 | N-tolyl | N-tolyl | H | H | H | b8 |
| 2-12-192 | N-tolyl | N-tolyl | t-bu | H | t-bu | b8 |
| 2-12-193 | N-tolyl | N-tolyl | methyl | H | methyl | b8 |
| 2-12-194 | N-tolyl | N-tolyl | b1 | H | b1 | b8 |
| 2-12-195 | N-tolyl | N-tolyl | b2 | H | b2 | b8 |
| 2-12-196 | N-tolyl | N-tolyl | b3 | H | b3 | b8 |
| 2-12-197 | N-tolyl | N-tolyl | H | H | H | b9 |
| 2-12-198 | N-tolyl | N-tolyl | t-bu | H | t-bu | b9 |
| 2-12-199 | N-tolyl | N-tolyl | methyl | H | methyl | b9 |
| 2-12-200 | N-tolyl | N-tolyl | b1 | H | b1 | b9 |
| 2-12-201 | N-tolyl | N-tolyl | b2 | H | b2 | b9 |
| 2-12-202 | N-tolyl | N-tolyl | b3 | H | b3 | b9 |
| 2-12-203 | N-tolyl | N-tolyl | H | H | H | b10 |
| 2-12-204 | N-tolyl | N-tolyl | t-bu | H | t-bu | b10 |
| 2-12-205 | N-tolyl | N-tolyl | methyl | H | methyl | b10 |
| 2-12-206 | N-tolyl | N-tolyl | b1 | H | b1 | b10 |
| 2-12-207 | N-tolyl | N-tolyl | b2 | H | b2 | b10 |
| 2-12-208 | N-tolyl | N-tolyl | b3 | H | b3 | b10 |
| 2-12-209 | N-Biph | N-Biph | H | H | H | H |
| 2-12-210 | N-Biph | N-Biph | t-bu | H | t-bu | H |
| 2-12-211 | N-Biph | N-Biph | methyl | H | methyl | H |
| 2-12-212 | N-Biph | N-Biph | t-bu | t-bu | t-bu | t-bu |
| 2-12-213 | N-Biph | N-Biph | methyl | methyl | methyl | methyl |
| 2-12-214 | N-Biph | N-Biph | b1 | H | b1 | H |
| 2-12-215 | N-Biph | N-Biph | b2 | H | b2 | H |
| 2-12-216 | N-Biph | N-Biph | b3 | H | b3 | H |
| 2-12-217 | N-Biph | N-Biph | H | H | H | a1 |
| 2-12-218 | N-Biph | N-Biph | t-bu | H | t-bu | a1 |
| 2-12-219 | N-Biph | N-Biph | methyl | H | methyl | a1 |
| 2-12-220 | N-Biph | N-Biph | b1 | H | b1 | a1 |
| 2-12-221 | N-Biph | N-Biph | b2 | H | b2 | a1 |
| 2-12-222 | N-Biph | N-Biph | b3 | H | b3 | a1 |
| 2-12-223 | N-Biph | N-Biph | H | H | H | a2 |
| 2-12-224 | N-Biph | N-Biph | t-bu | H | t-bu | a2 |
| 2-12-225 | N-Biph | N-Biph | methyl | H | methyl | a2 |
| 2-12-226 | N-Biph | N-Biph | b1 | H | b1 | a2 |
| 2-12-227 | N-Biph | N-Biph | b2 | H | b2 | a2 |
| 2-12-228 | N-Biph | N-Biph | b3 | H | b3 | a2 |
| 2-12-229 | N-Biph | N-Biph | H | H | H | a3 |
| 2-12-230 | N-Biph | N-Biph | t-bu | H | t-bu | a3 |
| 2-12-231 | N-Biph | N-Biph | methyl | H | methyl | a3 |
| 2-12-232 | N-Biph | N-Biph | b1 | H | b1 | a3 |
| 2-12-233 | N-Biph | N-Biph | b2 | H | b2 | a3 |
| 2-12-234 | N-Biph | N-Biph | b3 | H | b3 | a3 |
| 2-12-235 | N-Biph | N-Biph | H | H | H | a4 |
| 2-12-236 | N-Biph | N-Biph | t-bu | H | t-bu | a4 |
| 2-12-237 | N-Biph | N-Biph | methyl | H | methyl | a4 |
| 2-12-238 | N-Biph | N-Biph | b1 | H | b1 | a4 |
| 2-12-239 | N-Biph | N-Biph | b2 | H | b2 | a4 |
| 2-12-240 | N-Biph | N-Biph | b3 | H | b3 | a4 |
| 2-12-241 | N-Biph | N-Biph | H | H | H | a5 |
| 2-12-242 | N-Biph | N-Biph | t-bu | H | t-bu | a5 |
| 2-12-243 | N-Biph | N-Biph | methyl | H | methyl | a5 |
| 2-12-244 | N-Biph | N-Biph | b1 | H | b1 | a5 |
| 2-12-245 | N-Biph | N-Biph | b2 | H | b2 | a5 |
| 2-12-246 | N-Biph | N-Biph | b3 | H | b3 | a5 |
| 2-12-247 | N-Biph | N-Biph | H | H | H | a6 |
| 2-12-248 | N-Biph | N-Biph | t-bu | H | t-bu | a6 |
| 2-12-249 | N-Biph | N-Biph | methyl | H | methyl | a6 |
| 2-12-250 | N-Biph | N-Biph | b1 | H | b1 | a6 |
| 2-12-251 | N-Biph | N-Biph | b2 | H | b2 | a6 |
| 2-12-252 | N-Biph | N-Biph | b3 | H | b3 | a6 |
| 2-12-253 | N-Biph | N-Biph | H | H | H | b1 |
| 2-12-254 | N-Biph | N-Biph | t-bu | H | t-bu | b1 |
| 2-12-255 | N-Biph | N-Biph | methyl | H | methyl | b1 |
| 2-12-256 | N-Biph | N-Biph | b1 | H | b1 | b1 |
| 2-12-257 | N-Biph | N-Biph | b2 | H | b2 | b1 |
| 2-12-258 | N-Biph | N-Biph | b3 | H | b3 | b1 |
| 2-12-259 | N-Biph | N-Biph | H | H | H | b2 |
| 2-12-260 | N-Biph | N-Biph | t-bu | H | t-bu | b2 |
| 2-12-261 | N-Biph | N-Biph | methyl | H | methyl | b2 |
| 2-12-262 | N-Biph | N-Biph | b1 | H | b1 | b2 |
| 2-12-263 | N-Biph | N-Biph | b2 | H | b2 | b2 |
| 2-12-264 | N-Biph | N-Biph | b3 | H | b3 | b2 |
| 2-12-265 | N-Biph | N-Biph | H | H | H | b3 |
| 2-12-266 | N-Biph | N-Biph | t-bu | H | t-bu | b3 |
| 2-12-267 | N-Biph | N-Biph | methyl | H | methyl | b3 |
| 2-12-268 | N-Biph | N-Biph | b1 | H | b1 | b3 |
| 2-12-269 | N-Biph | N-Biph | b2 | H | b2 | b3 |
| 2-12-270 | N-Biph | N-Biph | b3 | H | b3 | b3 |
| 2-12-271 | N-Biph | N-Biph | H | H | H | b4 |
| 2-12-272 | N-Biph | N-Biph | t-bu | H | t-bu | b4 |
| 2-12-273 | N-Biph | N-Biph | methyl | H | methyl | b4 |
| 2-12-274 | N-Biph | N-Biph | b1 | H | b1 | b4 |
| 2-12-275 | N-Biph | N-Biph | b2 | H | b2 | b4 |
| 2-12-276 | N-Biph | N-Biph | b3 | H | b3 | b4 |
| 2-12-277 | N-Biph | N-Biph | H | H | H | b5 |
| 2-12-278 | N-Biph | N-Biph | t-bu | H | t-bu | b5 |
| 2-12-279 | N-Biph | N-Biph | methyl | H | methyl | b5 |
| 2-12-280 | N-Biph | N-Biph | b1 | H | b1 | b5 |
| 2-12-281 | N-Biph | N-Biph | b2 | H | b2 | b5 |
| 2-12-282 | N-Biph | N-Biph | b3 | H | b3 | b5 |
| 2-12-283 | N-Biph | N-Biph | H | H | H | b6 |
| 2-12-284 | N-Biph | N-Biph | t-bu | H | t-bu | b6 |
| 2-12-285 | N-Biph | N-Biph | methyl | H | methyl | b6 |
| 2-12-286 | N-Biph | N-Biph | b1 | H | b1 | b6 |
| 2-12-287 | N-Biph | N-Biph | b2 | H | b2 | b6 |
| 2-12-288 | N-Biph | N-Biph | b3 | H | b3 | b6 |
| 2-12-289 | N-Biph | N-Biph | H | H | H | b7 |
| 2-12-290 | N-Biph | N-Biph | t-bu | H | t-bu | b7 |
| 2-12-291 | N-Biph | N-Biph | methyl | H | methyl | b7 |
| 2-12-292 | N-Biph | N-Biph | b1 | H | b1 | b7 |
| 2-12-293 | N-Biph | N-Biph | b2 | H | b2 | b7 |
| 2-12-294 | N-Biph | N-Biph | b3 | H | b3 | b7 |
| 2-12-295 | N-Biph | N-Biph | H | H | H | b8 |
| 2-12-296 | N-Biph | N-Biph | t-bu | H | t-bu | b8 |
| 2-12-297 | N-Biph | N-Biph | methyl | H | methyl | b8 |
| 2-12-298 | N-Biph | N-Biph | b1 | H | b1 | b8 |
| 2-12-299 | N-Biph | N-Biph | b2 | H | b2 | b8 |
| 2-12-300 | N-Biph | N-Biph | b3 | H | b3 | b8 |
| 2-12-301 | N-Biph | N-Biph | H | H | H | b9 |
| 2-12-302 | N-Biph | N-Biph | t-bu | H | t-bu | b9 |
| 2-12-303 | N-Biph | N-Biph | methyl | H | methyl | b9 |
| 2-12-304 | N-Biph | N-Biph | b1 | H | b1 | b9 |
| 2-12-305 | N-Biph | N-Biph | b2 | H | b2 | b9 |
| 2-12-306 | N-Biph | N-Biph | b3 | H | b3 | b9 |
| 2-12-307 | N-Biph | N-Biph | H | H | H | b10 |
| 2-12-308 | N-Biph | N-Biph | t-bu | H | t-bu | b10 |
| 2-12-309 | N-Biph | N-Biph | methyl | H | methyl | b10 |
| 2-12-310 | N-Biph | N-Biph | b1 | H | b1 | b10 |
| 2-12-311 | N-Biph | N-Biph | b2 | H | b2 | b10 |
| 2-12-312 | N-Biph | N-Biph | b3 | H | b3 | b10 |
| 2-12-313 | O | N-Ph | H | H | H | H |
| 2-12-314 | O | N-Ph | t-bu | H | t-bu | H |
| 2-12-315 | O | N-Ph | methyl | H | methyl | H |
| 2-12-316 | O | N-Ph | t-bu | t-bu | t-bu | t-bu |
| 2-12-317 | O | N-Ph | methyl | methyl | methyl | methyl |
| 2-12-318 | O | N-Ph | b1 | H | b1 | H |
| 2-12-319 | O | N-Ph | b2 | H | b2 | H |
| 2-12-320 | O | N-Ph | b3 | H | b3 | H |
| 2-12-321 | O | N-Ph | H | H | H | a1 |
| 2-12-322 | O | N-Ph | t-bu | H | t-bu | a1 |
| 2-12-323 | O | N-Ph | methyl | H | methyl | a1 |
| 2-12-324 | O | N-Ph | b1 | H | b1 | a1 |
| 2-12-325 | O | N-Ph | b2 | H | b2 | a1 |
| 2-12-326 | O | N-Ph | b3 | H | b3 | a1 |
| 2-12-327 | O | N-Ph | H | H | H | a2 |
| 2-12-328 | O | N-Ph | t-bu | H | t-bu | a2 |
| 2-12-329 | O | N-Ph | methyl | H | methyl | a2 |
| 2-12-330 | O | N-Ph | b1 | H | b1 | a2 |
| 2-12-331 | O | N-Ph | b2 | H | b2 | a2 |
| 2-12-332 | O | N-Ph | b3 | H | b3 | a2 |
| 2-12-333 | O | N-Ph | H | H | H | a3 |
| 2-12-334 | O | N-Ph | t-bu | H | t-bu | a3 |
| 2-12-335 | O | N-Ph | methyl | H | methyl | a3 |
| 2-12-336 | O | N-Ph | b1 | H | b1 | a3 |
| 2-12-337 | O | N-Ph | b2 | H | b2 | a3 |
| 2-12-338 | O | N-Ph | b3 | H | b3 | a3 |
| 2-12-339 | O | N-Ph | H | H | H | a4 |
| 2-12-340 | O | N-Ph | t-bu | H | t-bu | a4 |
| 2-12-341 | O | N-Ph | methyl | H | methyl | a4 |
| 2-12-342 | O | N-Ph | b1 | H | b1 | a4 |
| 2-12-343 | O | N-Ph | b2 | H | b2 | a4 |
| 2-12-344 | O | N-Ph | b3 | H | b3 | a4 |
| 2-12-345 | O | N-Ph | H | H | H | a5 |
| 2-12-346 | O | N-Ph | t-bu | H | t-bu | a5 |
| 2-12-347 | O | N-Ph | methyl | H | methyl | a5 |
| 2-12-348 | O | N-Ph | b1 | H | b1 | a5 |
| 2-12-349 | O | N-Ph | b2 | H | b2 | a5 |
| 2-12-350 | O | N-Ph | b3 | H | b3 | a5 |
| 2-12-351 | O | N-Ph | H | H | H | a6 |
| 2-12-352 | O | N-Ph | t-bu | H | t-bu | a6 |
| 2-12-353 | O | N-Ph | methyl | H | methyl | a6 |
| 2-12-354 | O | N-Ph | b1 | H | b1 | a6 |
| 2-12-355 | O | N-Ph | b2 | H | b2 | a6 |
| 2-12-356 | O | N-Ph | b3 | H | b3 | a6 |
| 2-12-357 | O | N-Ph | H | H | H | b1 |
| 2-12-358 | O | N-Ph | t-bu | H | t-bu | b1 |
| 2-12-359 | O | N-Ph | methyl | H | methyl | b1 |
| 2-12-360 | O | N-Ph | b1 | H | b1 | b1 |
| 2-12-361 | O | N-Ph | b2 | H | b2 | b1 |
| 2-12-362 | O | N-Ph | b3 | H | b3 | b1 |
| 2-12-363 | O | N-Ph | H | H | H | b2 |
| 2-12-364 | O | N-Ph | t-bu | H | t-bu | b2 |
| 2-12-365 | O | N-Ph | methyl | H | methyl | b2 |
| 2-12-366 | O | N-Ph | b1 | H | b1 | b2 |
| 2-12-367 | O | N-Ph | b2 | H | b2 | b2 |
| 2-12-368 | O | N-Ph | b3 | H | b3 | b2 |
| 2-12-369 | O | N-Ph | H | H | H | b3 |
| 2-12-370 | O | N-Ph | t-bu | H | t-bu | b3 |
| 2-12-371 | O | N-Ph | methyl | H | methyl | b3 |
| 2-12-372 | O | N-Ph | b1 | H | b1 | b3 |
| 2-12-373 | O | N-Ph | b2 | H | b2 | b3 |
| 2-12-374 | O | N-Ph | b3 | H | b3 | b3 |
| 2-12-375 | O | N-Ph | H | H | H | b4 |
| 2-12-376 | O | N-Ph | t-bu | H | t-bu | b4 |
| 2-12-377 | O | N-Ph | methyl | H | methyl | b4 |
| 2-12-378 | O | N-Ph | b1 | H | b1 | b4 |
| 2-12-379 | O | N-Ph | b2 | H | b2 | b4 |
| 2-12-380 | O | N-Ph | b3 | H | b3 | b4 |
| 2-12-381 | O | N-Ph | H | H | H | b5 |
| 2-12-382 | O | N-Ph | t-bu | H | t-bu | b5 |
| 2-12-383 | O | N-Ph | methyl | H | methyl | b5 |
| 2-12-384 | O | N-Ph | b1 | H | b1 | b5 |
| 2-12-385 | O | N-Ph | b2 | H | b2 | b5 |
| 2-12-386 | O | N-Ph | b3 | H | b3 | b5 |
| 2-12-387 | O | N-Ph | H | H | H | b6 |
| 2-12-388 | O | N-Ph | t-bu | H | t-bu | b6 |
| 2-12-389 | O | N-Ph | methyl | H | methyl | b6 |
| 2-12-390 | O | N-Ph | b1 | H | b1 | b6 |
| 2-12-391 | O | N-Ph | b2 | H | b2 | b6 |
| 2-12-392 | O | N-Ph | b3 | H | b3 | b6 |
| 2-12-393 | O | N-Ph | H | H | H | b7 |
| 2-12-394 | O | N-Ph | t-bu | H | t-bu | b7 |
| 2-12-395 | O | N-Ph | methyl | H | methyl | b7 |
| 2-12-396 | O | N-Ph | b1 | H | b1 | b7 |
| 2-12-397 | O | N-Ph | b2 | H | b2 | b7 |
| 2-12-398 | O | N-Ph | b3 | H | b3 | b7 |
| 2-12-399 | O | N-Ph | H | H | H | b8 |
| 2-12-400 | O | N-Ph | t-bu | H | t-bu | b8 |
| 2-12-401 | O | N-Ph | methyl | H | methyl | b8 |
| 2-12-402 | O | N-Ph | b1 | H | b1 | b8 |
| 2-12-403 | O | N-Ph | b2 | H | b2 | b8 |
| 2-12-404 | O | N-Ph | b3 | H | b3 | b8 |
| 2-12-405 | O | N-Ph | H | H | H | b9 |
| 2-12-406 | O | N-Ph | t-bu | H | t-bu | b9 |
| 2-12-407 | O | N-Ph | methyl | H | methyl | b9 |
| 2-12-408 | O | N-Ph | b1 | H | b1 | b9 |
| 2-12-409 | O | N-Ph | b2 | H | b2 | b9 |
| 2-12-410 | O | N-Ph | b3 | H | b3 | b9 |
| 2-12-411 | O | N-Ph | H | H | H | b10 |
| 2-12-412 | O | N-Ph | t-bu | H | t-bu | b10 |
| 2-12-413 | O | N-Ph | methyl | H | methyl | b10 |
| 2-12-414 | O | N-Ph | b1 | H | b1 | b10 |
| 2-12-415 | O | N-Ph | b2 | H | b2 | b10 |
| 2-12-416 | O | N-Ph | b3 | H | b3 | b10 |
| 2-12-417 | S | N-Ph | H | H | H | H |
| 2-12-418 | S | N-Ph | t-bu | H | t-bu | H |
| 2-12-419 | S | N-Ph | methyl | H | methyl | H |
| 2-12-420 | S | N-Ph | t-bu | t-bu | t-bu | t-bu |
| 2-12-421 | S | N-Ph | methyl | methyl | methyl | methyl |
| 2-12-422 | S | N-Ph | b1 | H | b1 | H |
| 2-12-423 | S | N-Ph | b2 | H | b2 | H |
| 2-12-424 | S | N-Ph | b3 | H | b3 | H |
| 2-12-425 | S | N-Ph | H | H | H | a1 |
| 2-12-426 | S | N-Ph | t-bu | H | t-bu | a1 |
| 2-12-427 | S | N-Ph | methyl | H | methyl | a1 |
| 2-12-428 | S | N-Ph | b1 | H | b1 | a1 |
| 2-12-429 | S | N-Ph | b2 | H | b2 | a1 |
| 2-12-430 | S | N-Ph | b3 | H | b3 | a1 |
| 2-12-431 | S | N-Ph | H | H | H | a2 |
| 2-12-432 | S | N-Ph | t-bu | H | t-bu | a2 |
| 2-12-433 | S | N-Ph | methyl | H | methyl | a2 |
| 2-12-434 | S | N-Ph | b1 | H | b1 | a2 |
| 2-12-435 | S | N-Ph | b2 | H | b2 | a2 |
| 2-12-436 | S | N-Ph | b3 | H | b3 | a2 |
| 2-12-437 | S | N-Ph | H | H | H | a3 |
| 2-12-438 | S | N-Ph | t-bu | H | t-bu | a3 |
| 2-12-439 | S | N-Ph | methyl | H | methyl | a3 |
| 2-12-440 | S | N-Ph | b1 | H | b1 | a3 |
| 2-12-441 | S | N-Ph | b2 | H | b2 | a3 |
| 2-12-442 | S | N-Ph | b3 | H | b3 | a3 |
| 2-12-443 | S | N-Ph | H | H | H | a4 |
| 2-12-444 | S | N-Ph | t-bu | H | t-bu | a4 |
| 2-12-445 | S | N-Ph | methyl | H | methyl | a4 |
| 2-12-446 | S | N-Ph | b1 | H | b1 | a4 |
| 2-12-447 | S | N-Ph | b2 | H | b2 | a4 |
| 2-12-448 | S | N-Ph | b3 | H | b3 | a4 |
| 2-12-449 | S | N-Ph | H | H | H | a5 |
| 2-12-450 | S | N-Ph | t-bu | H | t-bu | a5 |
| 2-12-451 | S | N-Ph | methyl | H | methyl | a5 |
| 2-12-452 | S | N-Ph | b1 | H | b1 | a5 |
| 2-12-453 | S | N-Ph | b2 | H | b2 | a5 |
| 2-12-454 | S | N-Ph | b3 | H | b3 | a5 |
| 2-12-455 | S | N-Ph | H | H | H | a6 |
| 2-12-456 | S | N-Ph | t-bu | H | t-bu | a6 |
| 2-12-457 | S | N-Ph | methyl | H | methyl | a6 |
| 2-12-458 | S | N-Ph | b1 | H | b1 | a6 |
| 2-12-459 | S | N-Ph | b2 | H | b2 | a6 |
| 2-12-460 | S | N-Ph | b3 | H | b3 | a6 |
| 2-12-461 | S | N-Ph | H | H | H | b1 |
| 2-12-462 | S | N-Ph | t-bu | H | t-bu | b1 |
| 2-12-463 | S | N-Ph | methyl | H | methyl | b1 |
| 2-12-464 | S | N-Ph | b1 | H | b1 | b1 |
| 2-12-465 | S | N-Ph | b2 | H | b2 | b1 |
| 2-12-466 | S | N-Ph | b3 | H | b3 | b1 |
| 2-12-467 | S | N-Ph | H | H | H | b2 |
| 2-12-468 | S | N-Ph | t-bu | H | t-bu | b2 |
| 2-12-469 | S | N-Ph | methyl | H | methyl | b2 |
| 2-12-470 | S | N-Ph | b1 | H | b1 | b2 |
| 2-12-471 | S | N-Ph | b2 | H | b2 | b2 |
| 2-12-472 | S | N-Ph | b3 | H | b3 | b2 |
| 2-12-473 | S | N-Ph | H | H | H | b3 |
| 2-12-474 | S | N-Ph | t-bu | H | t-bu | b3 |
| 2-12-475 | S | N-Ph | methyl | H | methyl | b3 |
| 2-12-476 | S | N-Ph | b1 | H | b1 | b3 |
| 2-12-477 | S | N-Ph | b2 | H | b2 | b3 |
| 2-12-478 | S | N-Ph | b3 | H | b3 | b3 |
| 2-12-479 | S | N-Ph | H | H | H | b4 |
| 2-12-480 | S | N-Ph | t-bu | H | t-bu | b4 |
| 2-12-481 | S | N-Ph | methyl | H | methyl | b4 |
| 2-12-482 | S | N-Ph | b1 | H | b1 | b4 |
| 2-12-483 | S | N-Ph | b2 | H | b2 | b4 |
| 2-12-484 | S | N-Ph | b3 | H | b3 | b4 |
| 2-12-485 | S | N-Ph | H | H | H | b5 |
| 2-12-486 | S | N-Ph | t-bu | H | t-bu | b5 |
| 2-12-487 | S | N-Ph | methyl | H | methyl | b5 |
| 2-12-488 | S | N-Ph | b1 | H | b1 | b5 |
| 2-12-489 | S | N-Ph | b2 | H | b2 | b5 |
| 2-12-490 | S | N-Ph | b3 | H | b3 | b5 |
| 2-12-491 | S | N-Ph | H | H | H | b6 |
| 2-12-492 | S | N-Ph | t-bu | H | t-bu | b6 |
| 2-12-493 | S | N-Ph | methyl | H | methyl | b6 |
| 2-12-494 | S | N-Ph | b1 | H | b1 | b6 |
| 2-12-495 | S | N-Ph | b2 | H | b2 | b6 |
| 2-12-496 | S | N-Ph | b3 | H | b3 | b6 |
| 2-12-497 | S | N-Ph | H | H | H | b7 |
| 2-12-498 | S | N-Ph | t-bu | H | t-bu | b7 |
| 2-12-499 | S | N-Ph | methyl | H | methyl | b7 |
| 2-12-500 | S | N-Ph | b1 | H | b1 | b7 |
| 2-12-501 | S | N-Ph | b2 | H | b2 | b7 |
| 2-12-502 | S | N-Ph | b3 | H | b3 | b7 |
| 2-12-503 | S | N-Ph | H | H | H | b8 |
| 2-12-504 | S | N-Ph | t-bu | H | t-bu | b8 |
| 2-12-505 | S | N-Ph | methyl | H | methyl | b8 |
| 2-12-506 | S | N-Ph | b1 | H | b1 | b8 |
| 2-12-507 | S | N-Ph | b2 | H | b2 | b8 |
| 2-12-508 | S | N-Ph | b3 | H | b3 | b8 |
| 2-12-509 | S | N-Ph | H | H | H | b9 |
| 2-12-510 | S | N-Ph | t-bu | H | t-bu | b9 |
| 2-12-511 | S | N-Ph | methyl | H | methyl | b9 |
| 2-12-512 | S | N-Ph | b1 | H | b1 | b9 |
| 2-12-513 | S | N-Ph | b2 | H | b2 | b9 |
| 2-12-514 | S | N-Ph | b3 | H | b3 | b9 |
| 2-12-515 | S | N-Ph | H | H | H | b10 |
| 2-12-516 | S | N-Ph | t-bu | H | t-bu | b10 |
| 2-12-517 | S | N-Ph | methyl | H | methyl | b10 |
| 2-12-518 | S | N-Ph | b1 | H | b1 | b10 |
| 2-12-519 | S | N-Ph | b2 | H | b2 | b10 |
| 2-12-520 | S | N-Ph | b3 | H | b3 | b10 |

Group 2-13
The compounds are represented by the formula 2-13 below, wherein CY₁ is a benzene group, X₁ is B, and Y1, Y2, R2a, R2c, R3a, (R1)a1 are each shown in the table 13 below, 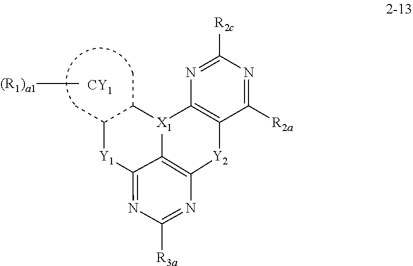
TABLE 13

| Compound | | | | | | |
|---|---|---|---|---|---|---|
| No. | Y1 | Y2 | R2a | R2c | R3a | (R1)a1 |
| 2-13-001 | N-Ph | N-Ph | H | H | H | H |
| 2-13-002 | N-Ph | N-Ph | t-bu | H | t-bu | H |
| 2-13-003 | N-Ph | N-Ph | methyl | H | methyl | H |
| 2-13-004 | N-Ph | N-Ph | t-bu | t-bu | t-bu | t-bu |
| 2-13-005 | N-Ph | N-Ph | methyl | methyl | methyl | methyl |
| 2-13-006 | N-Ph | N-Ph | b1 | H | b1 | H |
| 2-13-007 | N-Ph | N-Ph | b2 | H | b2 | H |
| 2-13-008 | N-Ph | N-Ph | b3 | H | b3 | H |
| 2-13-009 | N-Ph | N-Ph | H | H | H | a1 |
| 2-13-010 | N-Ph | N-Ph | t-bu | t-bu | t-bu | a1 |
| 2-13-011 | N-Ph | N-Ph | methyl | methyl | methyl | a1 |
| 2-13-012 | N-Ph | N-Ph | b1 | b1 | b1 | a1 |
| 2-13-013 | N-Ph | N-Ph | b2 | b2 | b2 | a1 |
| 2-13-014 | N-Ph | N-Ph | b3 | b3 | b3 | a1 |
| 2-13-015 | N-Ph | N-Ph | H | H | H | a2 |
| 2-13-016 | N-Ph | N-Ph | t-bu | t-bu | t-bu | a2 |
| 2-13-017 | N-Ph | N-Ph | methyl | methyl | methyl | a2 |
| 2-13-018 | N-Ph | N-Ph | b1 | b1 | b1 | a2 |
| 2-13-019 | N-Ph | N-Ph | b2 | b2 | b2 | a2 |
| 2-13-020 | N-Ph | N-Ph | b3 | b3 | b3 | a2 |
| 2-13-021 | N-Ph | N-Ph | H | H | H | a3 |
| 2-13-022 | N-Ph | N-Ph | t-bu | t-bu | t-bu | a3 |
| 2-13-023 | N-Ph | N-Ph | methyl | methyl | methyl | a3 |
| 2-13-024 | N-Ph | N-Ph | b1 | b1 | b1 | a3 |
| 2-13-025 | N-Ph | N-Ph | b2 | b2 | b2 | a3 |
| 2-13-026 | N-Ph | N-Ph | b3 | b3 | b3 | a3 |
| 2-13-027 | N-Ph | N-Ph | H | H | H | a4 |
| 2-13-028 | N-Ph | N-Ph | t-bu | t-bu | t-bu | a4 |
| 2-13-029 | N-Ph | N-Ph | methyl | methyl | methyl | a4 |
| 2-13-030 | N-Ph | N-Ph | b1 | b1 | b1 | a4 |
| 2-13-031 | N-Ph | N-Ph | b2 | b2 | b2 | a4 |
| 2-13-032 | N-Ph | N-Ph | b3 | b3 | b3 | a4 |
| 2-13-033 | N-Ph | N-Ph | H | H | H | a5 |
| 2-13-034 | N-Ph | N-Ph | t-bu | t-bu | t-bu | a5 |
| 2-13-035 | N-Ph | N-Ph | methyl | methyl | methyl | a5 |
| 2-13-036 | N-Ph | N-Ph | b1 | b1 | b1 | a5 |
| 2-13-037 | N-Ph | N-Ph | b2 | b2 | b2 | a5 |
| 2-13-038 | N-Ph | N-Ph | b3 | b3 | b3 | a5 |
| 2-13-039 | N-Ph | N-Ph | H | H | H | a6 |
| 2-13-040 | N-Ph | N-Ph | t-bu | t-bu | t-bu | a6 |
| 2-13-041 | N-Ph | N-Ph | methyl | methyl | methyl | a6 |
| 2-13-042 | N-Ph | N-Ph | b1 | b1 | b1 | a6 |
| 2-13-043 | N-Ph | N-Ph | b2 | b2 | b2 | a6 |
| 2-13-044 | N-Ph | N-Ph | b3 | b3 | b3 | a6 |
| 2-13-045 | N-Ph | N-Ph | H | H | H | b1 |
| 2-13-046 | N-Ph | N-Ph | t-bu | t-bu | t-bu | b1 |
| 2-13-047 | N-Ph | N-Ph | methyl | methyl | methyl | b1 |
| 2-13-048 | N-Ph | N-Ph | b1 | b1 | b1 | b1 |
| 2-13-049 | N-Ph | N-Ph | b2 | b2 | b2 | b1 |
| 2-13-050 | N-Ph | N-Ph | b3 | b3 | b3 | b1 |
| 2-13-051 | N-Ph | N-Ph | H | H | H | b2 |
| 2-13-052 | N-Ph | N-Ph | t-bu | t-bu | t-bu | b2 |
| 2-13-053 | N-Ph | N-Ph | methyl | methyl | methyl | b2 |
| 2-13-054 | N-Ph | N-Ph | b1 | b1 | b1 | b2 |
| 2-13-055 | N-Ph | N-Ph | b2 | b2 | b2 | b2 |
| 2-13-056 | N-Ph | N-Ph | b3 | b3 | b3 | b2 |
| 2-13-057 | N-Ph | N-Ph | H | H | H | b3 |
| 2-13-058 | N-Ph | N-Ph | t-bu | t-bu | t-bu | b3 |
| 2-13-059 | N-Ph | N-Ph | methyl | methyl | methyl | b3 |
| 2-13-060 | N-Ph | N-Ph | b1 | b1 | b1 | b3 |
| 2-13-061 | N-Ph | N-Ph | b2 | b2 | b2 | b3 |
| 2-13-062 | N-Ph | N-Ph | b3 | b3 | b3 | b3 |
| 2-13-063 | N-Ph | N-Ph | H | H | H | b4 |
| 2-13-064 | N-Ph | N-Ph | t-bu | t-bu | t-bu | b4 |
| 2-13-065 | N-Ph | N-Ph | methyl | methyl | methyl | b4 |
| 2-13-066 | N-Ph | N-Ph | b1 | b1 | b1 | b4 |
| 2-13-067 | N-Ph | N-Ph | b2 | b2 | b2 | b4 |
| 2-13-068 | N-Ph | N-Ph | b3 | b3 | b3 | b4 |
| 2-13-069 | N-Ph | N-Ph | H | H | H | b5 |
| 2-13-070 | N-Ph | N-Ph | t-bu | t-bu | t-bu | b5 |
| 2-13-071 | N-Ph | N-Ph | methyl | methyl | methyl | b5 |
| 2-13-072 | N-Ph | N-Ph | b1 | b1 | b1 | b5 |
| 2-13-073 | N-Ph | N-Ph | b2 | b2 | b2 | b5 |
| 2-13-074 | N-Ph | N-Ph | b3 | b3 | b3 | b5 |
| 2-13-075 | N-Ph | N-Ph | H | H | H | b6 |
| 2-13-076 | N-Ph | N-Ph | t-bu | t-bu | t-bu | b6 |
| 2-13-077 | N-Ph | N-Ph | methyl | methyl | methyl | b6 |
| 2-13-078 | N-Ph | N-Ph | b1 | b1 | b1 | b6 |
| 2-13-079 | N-Ph | N-Ph | b2 | b2 | b2 | b6 |
| 2-13-080 | N-Ph | N-Ph | b3 | b3 | b3 | b6 |
| 2-13-081 | N-Ph | N-Ph | H | H | H | b7 |
| 2-13-082 | N-Ph | N-Ph | t-bu | t-bu | t-bu | b7 |
| 2-13-083 | N-Ph | N-Ph | methyl | methyl | methyl | b7 |
| 2-13-084 | N-Ph | N-Ph | b1 | b1 | b1 | b7 |
| 2-13-085 | N-Ph | N-Ph | b2 | b2 | b2 | b7 |
| 2-13-086 | N-Ph | N-Ph | b3 | b3 | b3 | b7 |
| 2-13-087 | N-Ph | N-Ph | H | H | H | b8 |
| 2-13-088 | N-Ph | N-Ph | t-bu | t-bu | t-bu | b8 |
| 2-13-089 | N-Ph | N-Ph | methyl | methyl | methyl | b8 |
| 2-13-090 | N-Ph | N-Ph | b1 | b1 | b1 | b8 |
| 2-13-091 | N-Ph | N-Ph | b2 | b2 | b2 | b8 |
| 2-13-092 | N-Ph | N-Ph | b3 | b3 | b3 | b8 |
| 2-13-093 | N-Ph | N-Ph | H | H | H | b9 |
| 2-13-094 | N-Ph | N-Ph | t-bu | t-bu | t-bu | b9 |
| 2-13-095 | N-Ph | N-Ph | methyl | methyl | methyl | b9 |
| 2-13-096 | N-Ph | N-Ph | b1 | b1 | b1 | b9 |
| 2-13-097 | N-Ph | N-Ph | b2 | b2 | b2 | b9 |
| 2-13-098 | N-Ph | N-Ph | b3 | b3 | b3 | b9 |
| 2-13-099 | N-Ph | N-Ph | H | H | H | b10 |
| 2-13-100 | N-Ph | N-Ph | t-bu | t-bu | t-bu | b10 |
| 2-13-101 | N-Ph | N-Ph | methyl | methyl | methyl | b10 |
| 2-13-102 | N-Ph | N-Ph | b1 | b1 | b1 | b10 |
| 2-13-103 | N-Ph | N-Ph | b2 | b2 | b2 | b10 |
| 2-13-104 | N-Ph | N-Ph | b3 | b3 | b3 | b10 |
| 2-13-105 | N-tolyl | N-tolyl | H | H | H | H |
| 2-13-106 | N-tolyl | N-tolyl | t-bu | H | t-bu | H |
| 2-13-107 | N-tolyl | N-tolyl | methyl | H | methyl | H |
| 2-13-108 | N-tolyl | N-tolyl | t-bu | t-bu | t-bu | t-bu |
| 2-13-109 | N-tolyl | N-tolyl | methyl | methyl | methyl | methyl |
| 2-13-110 | N-tolyl | N-tolyl | b1 | H | b1 | H |
| 2-13-111 | N-tolyl | N-tolyl | b2 | H | b2 | H |
| 2-13-112 | N-tolyl | N-tolyl | b3 | H | b3 | H |
| 2-13-113 | N-tolyl | N-tolyl | H | H | H | a1 |
| 2-13-114 | N-tolyl | N-tolyl | t-bu | t-bu | t-bu | a1 |
| 2-13-115 | N-tolyl | N-tolyl | methyl | methyl | methyl | a1 |
| 2-13-116 | N-tolyl | N-tolyl | b1 | b1 | b1 | a1 |
| 2-13-117 | N-tolyl | N-tolyl | b2 | b2 | b2 | a1 |
| 2-13-118 | N-tolyl | N-tolyl | b3 | b3 | b3 | a1 |
| 2-13-119 | N-tolyl | N-tolyl | H | H | H | a2 |
| 2-13-120 | N-tolyl | N-tolyl | t-bu | t-bu | t-bu | a2 |
| 2-13-121 | N-tolyl | N-tolyl | methyl | methyl | methyl | a2 |
| 2-13-122 | N-tolyl | N-tolyl | b1 | b1 | b1 | a2 |
| 2-13-123 | N-tolyl | N-tolyl | b2 | b2 | b2 | a2 |
| 2-13-124 | N-tolyl | N-tolyl | b3 | b3 | b3 | a2 |
| 2-13-125 | N-tolyl | N-tolyl | H | H | H | a3 |
| 2-13-126 | N-tolyl | N-tolyl | t-bu | t-bu | t-bu | a3 |
| 2-13-127 | N-tolyl | N-tolyl | methyl | methyl | methyl | a3 |
| 2-13-128 | N-tolyl | N-tolyl | b1 | b1 | b1 | a3 |
| 2-13-129 | N-tolyl | N-tolyl | b2 | b2 | b2 | a3 |
| 2-13-130 | N-tolyl | N-tolyl | b3 | b3 | b3 | a3 |
| 2-13-131 | N-tolyl | N-tolyl | H | H | H | a4 |
| 2-13-132 | N-tolyl | N-tolyl | t-bu | t-bu | t-bu | a4 |
| 2-13-133 | N-tolyl | N-tolyl | methyl | methyl | methyl | a4 |
| 2-13-134 | N-tolyl | N-tolyl | b1 | b1 | b1 | a4 |
| 2-13-135 | N-tolyl | N-tolyl | b2 | b2 | b2 | a4 |
| 2-13-136 | N-tolyl | N-tolyl | b3 | b3 | b3 | a4 |
| 2-13-137 | N-tolyl | N-tolyl | H | H | H | a5 |
| 2-13-138 | N-tolyl | N-tolyl | t-bu | t-bu | t-bu | a5 |
| 2-13-139 | N-tolyl | N-tolyl | methyl | methyl | methyl | a5 |
| 2-13-140 | N-tolyl | N-tolyl | b1 | b1 | b1 | a5 |
| 2-13-141 | N-tolyl | N-tolyl | b2 | b2 | b2 | a5 |
| 2-13-142 | N-tolyl | N-tolyl | b3 | b3 | b3 | a5 |
| 2-13-143 | N-tolyl | N-tolyl | H | H | H | a6 |
| 2-13-144 | N-tolyl | N-tolyl | t-bu | t-bu | t-bu | a6 |
| 2-13-145 | N-tolyl | N-tolyl | methyl | methyl | methyl | a6 |
| 2-13-146 | N-tolyl | N-tolyl | b1 | b1 | b1 | a6 |
| 2-13-147 | N-tolyl | N-tolyl | b2 | b2 | b2 | a6 |
| 2-13-148 | N-tolyl | N-tolyl | b3 | b3 | b3 | a6 |
| 2-13-149 | N-tolyl | N-tolyl | H | H | H | b1 |
| 2-13-150 | N-tolyl | N-tolyl | t-bu | t-bu | t-bu | b1 |
| 2-13-151 | N-tolyl | N-tolyl | methyl | methyl | methyl | b1 |
| 2-13-152 | N-tolyl | N-tolyl | b1 | b1 | b1 | b1 |
| 2-13-153 | N-tolyl | N-tolyl | b2 | b2 | b2 | b1 |
| 2-13-154 | N-tolyl | N-tolyl | b3 | b3 | b3 | b1 |
| 2-13-155 | N-tolyl | N-tolyl | H | H | H | b2 |
| 2-13-156 | N-tolyl | N-tolyl | t-bu | t-bu | t-bu | b2 |
| 2-13-157 | N-tolyl | N-tolyl | methyl | methyl | methyl | b2 |
| 2-13-158 | N-tolyl | N-tolyl | b1 | b1 | b1 | b2 |
| 2-13-159 | N-tolyl | N-tolyl | b2 | b2 | b2 | b2 |
| 2-13-160 | N-tolyl | N-tolyl | b3 | b3 | b3 | b2 |
| 2-13-161 | N-tolyl | N-tolyl | H | H | H | b3 |
| 2-13-162 | N-tolyl | N-tolyl | t-bu | t-bu | t-bu | b3 |
| 2-13-163 | N-tolyl | N-tolyl | methyl | methyl | methyl | b3 |
| 2-13-164 | N-tolyl | N-tolyl | b1 | b1 | b1 | b3 |
| 2-13-165 | N-tolyl | N-tolyl | b2 | b2 | b2 | b3 |
| 2-13-166 | N-tolyl | N-tolyl | b3 | b3 | b3 | b3 |
| 2-13-167 | N-tolyl | N-tolyl | H | H | H | b4 |
| 2-13-168 | N-tolyl | N-tolyl | t-bu | t-bu | t-bu | b4 |
| 2-13-169 | N-tolyl | N-tolyl | methyl | methyl | methyl | b4 |
| 2-13-170 | N-tolyl | N-tolyl | b1 | b1 | b1 | b4 |
| 2-13-171 | N-tolyl | N-tolyl | b2 | b2 | b2 | b4 |
| 2-13-172 | N-tolyl | N-tolyl | b3 | b3 | b3 | b4 |
| 2-13-173 | N-tolyl | N-tolyl | H | H | H | b5 |
| 2-13-174 | N-tolyl | N-tolyl | t-bu | t-bu | t-bu | b5 |
| 2-13-175 | N-tolyl | N-tolyl | methyl | methyl | methyl | b5 |
| 2-13-176 | N-tolyl | N-tolyl | b1 | b1 | b1 | b5 |
| 2-13-177 | N-tolyl | N-tolyl | b2 | b2 | b2 | b5 |
| 2-13-178 | N-tolyl | N-tolyl | b3 | b3 | b3 | b5 |
| 2-13-179 | N-tolyl | N-tolyl | H | H | H | b6 |
| 2-13-180 | N-tolyl | N-tolyl | t-bu | t-bu | t-bu | b6 |
| 2-13-181 | N-tolyl | N-tolyl | methyl | methyl | methyl | b6 |
| 2-13-182 | N-tolyl | N-tolyl | b1 | b1 | b1 | b6 |
| 2-13-183 | N-tolyl | N-tolyl | b2 | b2 | b2 | b6 |
| 2-13-184 | N-tolyl | N-tolyl | b3 | b3 | b3 | b6 |
| 2-13-185 | N-tolyl | N-tolyl | H | H | H | b7 |
| 2-13-186 | N-tolyl | N-tolyl | t-bu | t-bu | t-bu | b7 |
| 2-13-187 | N-tolyl | N-tolyl | methyl | methyl | methyl | b7 |
| 2-13-188 | N-tolyl | N-tolyl | b1 | b1 | b1 | b7 |
| 2-13-189 | N-tolyl | N-tolyl | b2 | b2 | b2 | b7 |
| 2-13-190 | N-tolyl | N-tolyl | b3 | b3 | b3 | b7 |
| 2-13-191 | N-tolyl | N-tolyl | H | H | H | b8 |
| 2-13-192 | N-tolyl | N-tolyl | t-bu | t-bu | t-bu | b8 |
| 2-13-193 | N-tolyl | N-tolyl | methyl | methyl | methyl | b8 |
| 2-13-194 | N-tolyl | N-tolyl | b1 | b1 | b1 | b8 |
| 2-13-195 | N-tolyl | N-tolyl | b2 | b2 | b2 | b8 |
| 2-13-196 | N-tolyl | N-tolyl | b3 | b3 | b3 | b8 |
| 2-13-197 | N-tolyl | N-tolyl | H | H | H | b9 |
| 2-13-198 | N-tolyl | N-tolyl | t-bu | t-bu | t-bu | b9 |
| 2-13-199 | N-tolyl | N-tolyl | methyl | methyl | methyl | b9 |
| 2-13-200 | N-tolyl | N-tolyl | b1 | b1 | b1 | b9 |
| 2-13-201 | N-tolyl | N-tolyl | b2 | b2 | b2 | b9 |
| 2-13-202 | N-tolyl | N-tolyl | b3 | b3 | b3 | b9 |
| 2-13-203 | N-tolyl | N-tolyl | H | H | H | b10 |
| 2-13-204 | N-tolyl | N-tolyl | t-bu | t-bu | t-bu | b10 |
| 2-13-205 | N-tolyl | N-tolyl | methyl | methyl | methyl | b10 |
| 2-13-206 | N-tolyl | N-tolyl | b1 | b1 | b1 | b10 |
| 2-13-207 | N-tolyl | N-tolyl | b2 | b2 | b2 | b10 |
| 2-13-208 | N-tolyl | N-tolyl | b3 | b3 | b3 | b10 |
| 2-13-209 | N-Biph | N-Biph | H | H | H | H |
| 2-13-210 | N-Biph | N-Biph | t-bu | H | t-bu | H |
| 2-13-211 | N-Biph | N-Biph | methyl | H | methyl | H |
| 2-13-212 | N-Biph | N-Biph | t-bu | t-bu | t-bu | t-bu |
| 2-13-213 | N-Biph | N-Biph | methyl | methyl | methyl | methyl |
| 2-13-214 | N-Biph | N-Biph | b1 | H | b1 | H |
| 2-13-215 | N-Biph | N-Biph | b2 | H | b2 | H |
| 2-13-216 | N-Biph | N-Biph | b3 | H | b3 | H |
| 2-13-217 | N-Biph | N-Biph | H | H | H | a1 |
| 2-13-218 | N-Biph | N-Biph | t-bu | t-bu | t-bu | a1 |
| 2-13-219 | N-Biph | N-Biph | methyl | methyl | methyl | a1 |
| 2-13-220 | N-Biph | N-Biph | b1 | b1 | b1 | a1 |
| 2-13-221 | N-Biph | N-Biph | b2 | b2 | b2 | a1 |
| 2-13-222 | N-Biph | N-Biph | b3 | b3 | b3 | a1 |
| 2-13-223 | N-Biph | N-Biph | H | H | H | a2 |
| 2-13-224 | N-Biph | N-Biph | t-bu | t-bu | t-bu | a2 |
| 2-13-225 | N-Biph | N-Biph | methyl | methyl | methyl | a2 |
| 2-13-226 | N-Biph | N-Biph | b1 | b1 | b1 | a2 |
| 2-13-227 | N-Biph | N-Biph | b2 | b2 | b2 | a2 |
| 2-13-228 | N-Biph | N-Biph | b3 | b3 | b3 | a2 |
| 2-13-229 | N-Biph | N-Biph | H | H | H | a3 |
| 2-13-230 | N-Biph | N-Biph | t-bu | t-bu | t-bu | a3 |
| 2-13-231 | N-Biph | N-Biph | methyl | methyl | methyl | a3 |
| 2-13-232 | N-Biph | N-Biph | b1 | b1 | b1 | a3 |
| 2-13-233 | N-Biph | N-Biph | b2 | b2 | b2 | a3 |
| 2-13-234 | N-Biph | N-Biph | b3 | b3 | b3 | a3 |
| 2-13-235 | N-Biph | N-Biph | H | H | H | a4 |
| 2-13-236 | N-Biph | N-Biph | t-bu | t-bu | t-bu | a4 |
| 2-13-237 | N-Biph | N-Biph | methyl | methyl | methyl | a4 |
| 2-13-238 | N-Biph | N-Biph | b1 | b1 | b1 | a4 |
| 2-13-239 | N-Biph | N-Biph | b2 | b2 | b2 | a4 |
| 2-13-240 | N-Biph | N-Biph | b3 | b3 | b3 | a4 |
| 2-13-241 | N-Biph | N-Biph | H | H | H | a5 |
| 2-13-242 | N-Biph | N-Biph | t-bu | t-bu | t-bu | a5 |
| 2-13-243 | N-Biph | N-Biph | methyl | methyl | methyl | a5 |
| 2-13-244 | N-Biph | N-Biph | b1 | b1 | b1 | a5 |
| 2-13-245 | N-Biph | N-Biph | b2 | b2 | b2 | a5 |
| 2-13-246 | N-Biph | N-Biph | b3 | b3 | b3 | a5 |
| 2-13-247 | N-Biph | N-Biph | H | H | H | a6 |
| 2-13-248 | N-Biph | N-Biph | t-bu | t-bu | t-bu | a6 |
| 2-13-249 | N-Biph | N-Biph | methyl | methyl | methyl | a6 |
| 2-13-250 | N-Biph | N-Biph | b1 | b1 | b1 | a6 |
| 2-13-251 | N-Biph | N-Biph | b2 | b2 | b2 | a6 |
| 2-13-252 | N-Biph | N-Biph | b3 | b3 | b3 | a6 |
| 2-13-253 | N-Biph | N-Biph | H | H | H | b1 |
| 2-13-254 | N-Biph | N-Biph | t-bu | t-bu | t-bu | b1 |
| 2-13-255 | N-Biph | N-Biph | methyl | methyl | methyl | b1 |
| 2-13-256 | N-Biph | N-Biph | b1 | b1 | b1 | b1 |
| 2-13-257 | N-Biph | N-Biph | b2 | b2 | b2 | b1 |
| 2-13-258 | N-Biph | N-Biph | b3 | b3 | b3 | b1 |
| 2-13-259 | N-Biph | N-Biph | H | H | H | b2 |
| 2-13-260 | N-Biph | N-Biph | t-bu | t-bu | t-bu | b2 |
| 2-13-261 | N-Biph | N-Biph | methyl | methyl | methyl | b2 |
| 2-13-262 | N-Biph | N-Biph | b1 | b1 | b1 | b2 |
| 2-13-263 | N-Biph | N-Biph | b2 | b2 | b2 | b2 |
| 2-13-264 | N-Biph | N-Biph | b3 | b3 | b3 | b2 |
| 2-13-265 | N-Biph | N-Biph | H | H | H | b3 |
| 2-13-266 | N-Biph | N-Biph | t-bu | t-bu | t-bu | b3 |
| 2-13-267 | N-Biph | N-Biph | methyl | methyl | methyl | b3 |
| 2-13-268 | N-Biph | N-Biph | b1 | b1 | b1 | b3 |
| 2-13-269 | N-Biph | N-Biph | b2 | b2 | b2 | b3 |
| 2-13-270 | N-Biph | N-Biph | b3 | b3 | b3 | b3 |
| 2-13-271 | N-Biph | N-Biph | H | H | H | b4 |
| 2-13-272 | N-Biph | N-Biph | t-bu | t-bu | t-bu | b4 |
| 2-13-273 | N-Biph | N-Biph | methyl | methyl | methyl | b4 |
| 2-13-274 | N-Biph | N-Biph | b1 | b1 | b1 | b4 |
| 2-13-275 | N-Biph | N-Biph | b2 | b2 | b2 | b4 |
| 2-13-276 | N-Biph | N-Biph | b3 | b3 | b3 | b4 |
| 2-13-277 | N-Biph | N-Biph | H | H | H | b5 |
| 2-13-278 | N-Biph | N-Biph | t-bu | t-bu | t-bu | b5 |
| 2-13-279 | N-Biph | N-Biph | methyl | methyl | methyl | b5 |
| 2-13-280 | N-Biph | N-Biph | b1 | b1 | b1 | b5 |
| 2-13-281 | N-Biph | N-Biph | b2 | b2 | b2 | b5 |
| 2-13-282 | N-Biph | N-Biph | b3 | b3 | b3 | b5 |
| 2-13-283 | N-Biph | N-Biph | H | H | H | b6 |
| 2-13-284 | N-Biph | N-Biph | t-bu | t-bu | t-bu | b6 |
| 2-13-285 | N-Biph | N-Biph | methyl | methyl | methyl | b6 |
| 2-13-286 | N-Biph | N-Biph | b1 | b1 | b1 | b6 |
| 2-13-287 | N-Biph | N-Biph | b2 | b2 | b2 | b6 |
| 2-13-288 | N-Biph | N-Biph | b3 | b3 | b3 | b6 |
| 2-13-289 | N-Biph | N-Biph | H | H | H | b7 |
| 2-13-290 | N-Biph | N-Biph | t-bu | t-bu | t-bu | b7 |
| 2-13-291 | N-Biph | N-Biph | methyl | methyl | methyl | b7 |
| 2-13-292 | N-Biph | N-Biph | b1 | b1 | b1 | b7 |
| 2-13-293 | N-Biph | N-Biph | b2 | b2 | b2 | b7 |
| 2-13-294 | N-Biph | N-Biph | b3 | b3 | b3 | b7 |
| 2-13-295 | N-Biph | N-Biph | H | H | H | b8 |
| 2-13-296 | N-Biph | N-Biph | t-bu | t-bu | t-bu | b8 |
| 2-13-297 | N-Biph | N-Biph | methyl | methyl | methyl | b8 |
| 2-13-298 | N-Biph | N-Biph | b1 | b1 | b1 | b8 |
| 2-13-299 | N-Biph | N-Biph | b2 | b2 | b2 | b8 |
| 2-13-300 | N-Biph | N-Biph | b3 | b3 | b3 | b8 |
| 2-13-301 | N-Biph | N-Biph | H | H | H | b9 |
| 2-13-302 | N-Biph | N-Biph | t-bu | t-bu | t-bu | b9 |
| 2-13-303 | N-Biph | N-Biph | methyl | methyl | methyl | b9 |
| 2-13-304 | N-Biph | N-Biph | b1 | b1 | b1 | b9 |
| 2-13-305 | N-Biph | N-Biph | b2 | b2 | b2 | b9 |
| 2-13-306 | N-Biph | N-Biph | b3 | b3 | b3 | b9 |
| 2-13-307 | N-Biph | N-Biph | H | H | H | b10 |
| 2-13-308 | N-Biph | N-Biph | t-bu | t-bu | t-bu | b10 |
| 2-13-309 | N-Biph | N-Biph | methyl | methyl | methyl | b10 |
| 2-13-310 | N-Biph | N-Biph | b1 | b1 | b1 | b10 |
| 2-13-311 | N-Biph | N-Biph | b2 | b2 | b2 | b10 |
| 2-13-312 | N-Biph | N-Biph | b3 | b3 | b3 | b10 |
| 2-13-313 | O | N-Ph | H | H | H | H |
| 2-13-314 | O | N-Ph | t-bu | H | t-bu | H |
| 2-13-315 | O | N-Ph | methyl | H | methyl | H |
| 2-13-316 | O | N-Ph | t-bu | t-bu | t-bu | t-bu |
| 2-13-317 | O | N-Ph | methyl | methyl | methyl | methyl |
| 2-13-318 | O | N-Ph | b1 | H | b1 | H |
| 2-13-319 | O | N-Ph | b2 | H | b2 | H |
| 2-13-320 | O | N-Ph | b3 | H | b3 | H |
| 2-13-321 | O | N-Ph | H | H | H | a1 |
| 2-13-322 | O | N-Ph | t-bu | t-bu | t-bu | a1 |
| 2-13-323 | O | N-Ph | methyl | methyl | methyl | a1 |
| 2-13-324 | O | N-Ph | b1 | b1 | b1 | a1 |
| 2-13-325 | O | N-Ph | b2 | b2 | b2 | a1 |
| 2-13-326 | O | N-Ph | b3 | b3 | b3 | a1 |
| 2-13-327 | O | N-Ph | H | H | H | a2 |
| 2-13-328 | O | N-Ph | t-bu | t-bu | t-bu | a2 |
| 2-13-329 | O | N-Ph | methyl | methyl | methyl | a2 |
| 2-13-330 | O | N-Ph | b1 | b1 | b1 | a2 |
| 2-13-331 | O | N-Ph | b2 | b2 | b2 | a2 |
| 2-13-332 | O | N-Ph | b3 | b3 | b3 | a2 |
| 2-13-333 | O | N-Ph | H | H | H | a3 |
| 2-13-334 | O | N-Ph | t-bu | t-bu | t-bu | a3 |
| 2-13-335 | O | N-Ph | methyl | methyl | methyl | a3 |
| 2-13-336 | O | N-Ph | b1 | b1 | b1 | a3 |
| 2-13-337 | O | N-Ph | b2 | b2 | b2 | a3 |
| 2-13-338 | O | N-Ph | b3 | b3 | b3 | a3 |
| 2-13-339 | O | N-Ph | H | H | H | a4 |
| 2-13-340 | O | N-Ph | t-bu | t-bu | t-bu | a4 |
| 2-13-341 | O | N-Ph | methyl | methyl | methyl | a4 |
| 2-13-342 | O | N-Ph | b1 | b1 | b1 | a4 |
| 2-13-343 | O | N-Ph | b2 | b2 | b2 | a4 |
| 2-13-344 | O | N-Ph | b3 | b3 | b3 | a4 |
| 2-13-345 | O | N-Ph | H | H | H | a5 |
| 2-13-346 | O | N-Ph | t-bu | t-bu | t-bu | a5 |
| 2-13-347 | O | N-Ph | methyl | methyl | methyl | a5 |
| 2-13-348 | O | N-Ph | b1 | b1 | b1 | a5 |
| 2-13-349 | O | N-Ph | b2 | b2 | b2 | a5 |
| 2-13-350 | O | N-Ph | b3 | b3 | b3 | a5 |
| 2-13-351 | O | N-Ph | H | H | H | a6 |
| 2-13-352 | O | N-Ph | t-bu | t-bu | t-bu | a6 |
| 2-13-353 | O | N-Ph | methyl | methyl | methyl | a6 |
| 2-13-354 | O | N-Ph | b1 | b1 | b1 | a6 |
| 2-13-355 | O | N-Ph | b2 | b2 | b2 | a6 |
| 2-13-356 | O | N-Ph | b3 | b3 | b3 | a6 |
| 2-13-357 | O | N-Ph | H | H | H | b1 |
| 2-13-358 | O | N-Ph | t-bu | t-bu | t-bu | b1 |
| 2-13-359 | O | N-Ph | methyl | methyl | methyl | b1 |
| 2-13-360 | O | N-Ph | b1 | b1 | b1 | b1 |
| 2-13-361 | O | N-Ph | b2 | b2 | b2 | b1 |
| 2-13-362 | O | N-Ph | b3 | b3 | b3 | b1 |
| 2-13-363 | O | N-Ph | H | H | H | b2 |
| 2-13-364 | O | N-Ph | t-bu | t-bu | t-bu | b2 |
| 2-13-365 | O | N-Ph | methyl | methyl | methyl | b2 |
| 2-13-366 | O | N-Ph | b1 | b1 | b1 | b2 |
| 2-13-367 | O | N-Ph | b2 | b2 | b2 | b2 |
| 2-13-368 | O | N-Ph | b3 | b3 | b3 | b2 |
| 2-13-369 | O | N-Ph | H | H | H | b3 |
| 2-13-370 | O | N-Ph | t-bu | t-bu | t-bu | b3 |
| 2-13-371 | O | N-Ph | methyl | methyl | methyl | b3 |
| 2-13-372 | O | N-Ph | b1 | b1 | b1 | b3 |
| 2-13-373 | O | N-Ph | b2 | b2 | b2 | b3 |
| 2-13-374 | O | N-Ph | b3 | b3 | b3 | b3 |
| 2-13-375 | O | N-Ph | H | H | H | b4 |
| 2-13-376 | O | N-Ph | t-bu | t-bu | t-bu | b4 |
| 2-13-377 | O | N-Ph | methyl | methyl | methyl | b4 |
| 2-13-378 | O | N-Ph | b1 | b1 | b1 | b4 |
| 2-13-379 | O | N-Ph | b2 | b2 | b2 | b4 |
| 2-13-380 | O | N-Ph | b3 | b3 | b3 | b4 |
| 2-13-381 | O | N-Ph | H | H | H | b5 |
| 2-13-382 | O | N-Ph | t-bu | t-bu | t-bu | b5 |
| 2-13-383 | O | N-Ph | methyl | methyl | methyl | b5 |
| 2-13-384 | O | N-Ph | b1 | b1 | b1 | b5 |
| 2-13-385 | O | N-Ph | b2 | b2 | b2 | b5 |
| 2-13-386 | O | N-Ph | b3 | b3 | b3 | b5 |
| 2-13-387 | O | N-Ph | H | H | H | b6 |
| 2-13-388 | O | N-Ph | t-bu | t-bu | t-bu | b6 |
| 2-13-389 | O | N-Ph | methyl | methyl | methyl | b6 |
| 2-13-390 | O | N-Ph | b1 | b1 | b1 | b6 |
| 2-13-391 | O | N-Ph | b2 | b2 | b2 | b6 |
| 2-13-392 | O | N-Ph | b3 | b3 | b3 | b6 |
| 2-13-393 | O | N-Ph | H | H | H | b7 |
| 2-13-394 | O | N-Ph | t-bu | t-bu | t-bu | b7 |
| 2-13-395 | O | N-Ph | methyl | methyl | methyl | b7 |
| 2-13-396 | O | N-Ph | b1 | b1 | b1 | b7 |
| 2-13-397 | O | N-Ph | b2 | b2 | b2 | b7 |
| 2-13-398 | O | N-Ph | b3 | b3 | b3 | b7 |
| 2-13-399 | O | N-Ph | H | H | H | b8 |
| 2-13-400 | O | N-Ph | t-bu | t-bu | t-bu | b8 |
| 2-13-401 | O | N-Ph | methyl | methyl | methyl | b8 |
| 2-13-402 | O | N-Ph | b1 | b1 | b1 | b8 |
| 2-13-403 | O | N-Ph | b2 | b2 | b2 | b8 |
| 2-13-404 | O | N-Ph | b3 | b3 | b3 | b8 |
| 2-13-405 | O | N-Ph | H | H | H | b9 |
| 2-13-406 | O | N-Ph | t-bu | t-bu | t-bu | b9 |
| 2-13-407 | O | N-Ph | methyl | methyl | methyl | b9 |
| 2-13-408 | O | N-Ph | b1 | b1 | b1 | b9 |
| 2-13-409 | O | N-Ph | b2 | b2 | b2 | b9 |
| 2-13-410 | O | N-Ph | b3 | b3 | b3 | b9 |
| 2-13-411 | O | N-Ph | H | H | H | b10 |
| 2-13-412 | O | N-Ph | t-bu | t-bu | t-bu | b10 |
| 2-13-413 | O | N-Ph | methyl | methyl | methyl | b10 |
| 2-13-414 | O | N-Ph | b1 | b1 | b1 | b10 |
| 2-13-415 | O | N-Ph | b2 | b2 | b2 | b10 |
| 2-13-416 | O | N-Ph | b3 | b3 | b3 | b10 |
| 2-13-417 | S | N-Ph | H | H | H | H |
| 2-13-418 | S | N-Ph | t-bu | H | t-bu | H |
| 2-13-419 | S | N-Ph | methyl | H | methyl | H |
| 2-13-420 | S | N-Ph | t-bu | t-bu | t-bu | t-bu |
| 2-13-421 | S | N-Ph | methyl | methyl | methyl | methyl |
| 2-13-422 | S | N-Ph | b1 | H | b1 | H |
| 2-13-423 | S | N-Ph | b2 | H | b2 | H |
| 2-13-424 | S | N-Ph | b3 | H | b3 | H |
| 2-13-425 | S | N-Ph | H | H | H | a1 |
| 2-13-426 | S | N-Ph | t-bu | t-bu | t-bu | a1 |
| 2-13-427 | S | N-Ph | methyl | methyl | methyl | a1 |
| 2-13-428 | S | N-Ph | b1 | b1 | b1 | a1 |
| 2-13-429 | S | N-Ph | b2 | b2 | b2 | a1 |
| 2-13-430 | S | N-Ph | b3 | b3 | b3 | a1 |
| 2-13-431 | S | N-Ph | H | H | H | a2 |
| 2-13-432 | S | N-Ph | t-bu | t-bu | t-bu | a2 |
| 2-13-433 | S | N-Ph | methyl | methyl | methyl | a2 |
| 2-13-434 | S | N-Ph | b1 | b1 | b1 | a2 |
| 2-13-435 | S | N-Ph | b2 | b2 | b2 | a2 |
| 2-13-436 | S | N-Ph | b3 | b3 | b3 | a2 |
| 2-13-437 | S | N-Ph | H | H | H | a3 |
| 2-13-438 | S | N-Ph | t-bu | t-bu | t-bu | a3 |
| 2-13-439 | S | N-Ph | methyl | methyl | methyl | a3 |
| 2-13-440 | S | N-Ph | b1 | b1 | b1 | a3 |
| 2-13-441 | S | N-Ph | b2 | b2 | b2 | a3 |
| 2-13-442 | S | N-Ph | b3 | b3 | b3 | a3 |
| 2-13-443 | S | N-Ph | H | H | H | a4 |
| 2-13-444 | S | N-Ph | t-bu | t-bu | t-bu | a4 |
| 2-13-445 | S | N-Ph | methyl | methyl | methyl | a4 |
| 2-13-446 | S | N-Ph | b1 | b1 | b1 | a4 |
| 2-13-447 | S | N-Ph | b2 | b2 | b2 | a4 |
| 2-13-448 | S | N-Ph | b3 | b3 | b3 | a4 |
| 2-13-449 | S | N-Ph | H | H | H | a5 |
| 2-13-450 | S | N-Ph | t-bu | t-bu | t-bu | a5 |
| 2-13-451 | S | N-Ph | methyl | methyl | methyl | a5 |
| 2-13-452 | S | N-Ph | b1 | b1 | b1 | a5 |
| 2-13-453 | S | N-Ph | b2 | b2 | b2 | a5 |
| 2-13-454 | S | N-Ph | b3 | b3 | b3 | a5 |
| 2-13-455 | S | N-Ph | H | H | H | a6 |
| 2-13-456 | S | N-Ph | t-bu | t-bu | t-bu | a6 |
| 2-13-457 | S | N-Ph | methyl | methyl | methyl | a6 |
| 2-13-458 | S | N-Ph | b1 | b1 | b1 | a6 |
| 2-13-459 | S | N-Ph | b2 | b2 | b2 | a6 |
| 2-13-460 | S | N-Ph | b3 | b3 | b3 | a6 |
| 2-13-461 | S | N-Ph | H | H | H | b1 |
| 2-13-462 | S | N-Ph | t-bu | t-bu | t-bu | b1 |
| 2-13-463 | S | N-Ph | methyl | methyl | methyl | b1 |
| 2-13-464 | S | N-Ph | b1 | b1 | b1 | b1 |
| 2-13-465 | S | N-Ph | b2 | b2 | b2 | b1 |
| 2-13-466 | S | N-Ph | b3 | b3 | b3 | b1 |
| 2-13-467 | S | N-Ph | H | H | H | b2 |
| 2-13-468 | S | N-Ph | t-bu | t-bu | t-bu | b2 |
| 2-13-469 | S | N-Ph | methyl | methyl | methyl | b2 |
| 2-13-470 | S | N-Ph | b1 | b1 | b1 | b2 |
| 2-13-471 | S | N-Ph | b2 | b2 | b2 | b2 |
| 2-13-472 | S | N-Ph | b3 | b3 | b3 | b2 |
| 2-13-473 | S | N-Ph | H | H | H | b3 |
| 2-13-474 | S | N-Ph | t-bu | t-bu | t-bu | b3 |
| 2-13-475 | S | N-Ph | methyl | methyl | methyl | b3 |
| 2-13-476 | S | N-Ph | b1 | b1 | b1 | b3 |
| 2-13-477 | S | N-Ph | b2 | b2 | b2 | b3 |
| 2-13-478 | S | N-Ph | b3 | b3 | b3 | b3 |
| 2-13-479 | S | N-Ph | H | H | H | b4 |
| 2-13-480 | S | N-Ph | t-bu | t-bu | t-bu | b4 |
| 2-13-481 | S | N-Ph | methyl | methyl | methyl | b4 |
| 2-13-482 | S | N-Ph | b1 | b1 | b1 | b4 |
| 2-13-483 | S | N-Ph | b2 | b2 | b2 | b4 |
| 2-13-484 | S | N-Ph | b3 | b3 | b3 | b4 |
| 2-13-485 | S | N-Ph | H | H | H | b5 |
| 2-13-486 | S | N-Ph | t-bu | t-bu | t-bu | b5 |
| 2-13-487 | S | N-Ph | methyl | methyl | methyl | b5 |
| 2-13-488 | S | N-Ph | b1 | b1 | b1 | b5 |
| 2-13-489 | S | N-Ph | b2 | b2 | b2 | b5 |
| 2-13-490 | S | N-Ph | b3 | b3 | b3 | b5 |
| 2-13-491 | S | N-Ph | H | H | H | b6 |
| 2-13-492 | S | N-Ph | t-bu | t-bu | t-bu | b6 |
| 2-13-493 | S | N-Ph | methyl | methyl | methyl | b6 |
| 2-13-494 | S | N-Ph | b1 | b1 | b1 | b6 |
| 2-13-495 | S | N-Ph | b2 | b2 | b2 | b6 |
| 2-13-496 | S | N-Ph | b3 | b3 | b3 | b6 |
| 2-13-497 | S | N-Ph | H | H | H | b7 |
| 2-13-498 | S | N-Ph | t-bu | t-bu | t-bu | b7 |
| 2-13-499 | S | N-Ph | methyl | methyl | methyl | b7 |
| 2-13-500 | S | N-Ph | b1 | b1 | b1 | b7 |
| 2-13-501 | S | N-Ph | b2 | b2 | b2 | b7 |
| 2-13-502 | S | N-Ph | b3 | b3 | b3 | b7 |
| 2-13-503 | S | N-Ph | H | H | H | b8 |
| 2-13-504 | S | N-Ph | t-bu | t-bu | t-bu | b8 |
| 2-13-505 | S | N-Ph | methyl | methyl | methyl | b8 |
| 2-13-506 | S | N-Ph | b1 | b1 | b1 | b8 |
| 2-13-507 | S | N-Ph | b2 | b2 | b2 | b8 |
| 2-13-508 | S | N-Ph | b3 | b3 | b3 | b8 |
| 2-13-509 | S | N-Ph | H | H | H | b9 |
| 2-13-510 | S | N-Ph | t-bu | t-bu | t-bu | b9 |
| 2-13-511 | S | N-Ph | methyl | methyl | methyl | b9 |
| 2-13-512 | S | N-Ph | b1 | b1 | b1 | b9 |
| 2-13-513 | S | N-Ph | b2 | b2 | b2 | b9 |
| 2-13-514 | S | N-Ph | b3 | b3 | b3 | b9 |
| 2-13-515 | S | N-Ph | H | H | H | b10 |
| 2-13-516 | S | N-Ph | t-bu | t-bu | t-bu | b10 |
| 2-13-517 | S | N-Ph | methyl | methyl | methyl | b10 |
| 2-13-518 | S | N-Ph | b1 | b1 | b1 | b10 |
| 2-13-519 | S | N-Ph | b2 | b2 | b2 | b10 |
| 2-13-520 | S | N-Ph | b3 | b3 | b3 | b10 |

Group 2-14
The compounds are represented by the formula 2-14 below, wherein X₁ is B, and Y1, Y2, R1b, R1d, R2b, R2d, R3a are each shown in the table 14 below, 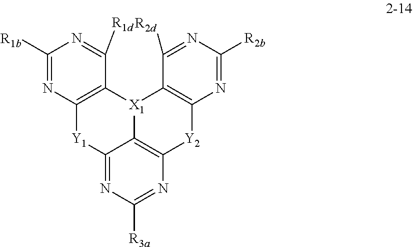
TABLE 14

| Compound | | | | | | | |
|---|---|---|---|---|---|---|---|
| No. | Y1 | Y2 | R1b | R1d | R2b | R2d | R3a |
| 2-14-001 | N-Ph | N-Ph | H | H | H | H | H |
| 2-14-002 | N-Ph | N-Ph | t-bu | H | t-bu | H | H |
| 2-14-003 | N-Ph | N-Ph | methyl | H | methyl | H | H |
| 2-14-004 | N-Ph | N-Ph | t-bu | t-bu | t-bu | t-bu | t-bu |
| 2-14-005 | N-Ph | N-Ph | methyl | methyl | methyl | methyl | methyl |
| 2-14-006 | N-Ph | N-Ph | b1 | H | b1 | H | H |
| 2-14-007 | N-Ph | N-Ph | b2 | H | b2 | H | H |
| 2-14-008 | N-Ph | N-Ph | b3 | H | b3 | H | H |
| 2-14-009 | N-Ph | N-Ph | t-bu | H | t-bu | H | t-bu |
| 2-14-010 | N-Ph | N-Ph | methyl | H | methyl | H | methyl |
| 2-14-011 | N-Ph | N-Ph | b1 | H | b1 | H | b1 |
| 2-14-012 | N-Ph | N-Ph | b2 | H | b2 | H | b2 |
| 2-14-013 | N-Ph | N-Ph | b3 | H | b3 | H | b3 |
| 2-14-014 | N-tolyl | N-tolyl | H | H | H | H | H |
| 2-14-015 | N-tolyl | N-tolyl | t-bu | H | t-bu | H | H |
| 2-14-016 | N-tolyl | N-tolyl | methyl | H | methyl | H | H |
| 2-14-017 | N-tolyl | N-tolyl | t-bu | t-bu | t-bu | t-bu | t-bu |
| 2-14-018 | N-tolyl | N-tolyl | methyl | methyl | methyl | methyl | methyl |
| 2-14-019 | N-tolyl | N-tolyl | b1 | H | b1 | H | H |
| 2-14-020 | N-tolyl | N-tolyl | b2 | H | b2 | H | H |
| 2-14-021 | N-tolyl | N-tolyl | b3 | H | b3 | H | H |
| 2-14-022 | N-tolyl | N-tolyl | t-bu | H | t-bu | H | t-bu |
| 2-14-023 | N-tolyl | N-tolyl | methyl | H | methyl | H | methyl |
| 2-14-024 | N-tolyl | N-tolyl | b1 | H | b1 | H | b1 |
| 2-14-025 | N-tolyl | N-tolyl | b2 | H | b2 | H | b2 |
| 2-14-026 | N-tolyl | N-tolyl | b3 | H | b3 | H | b3 |
| 2-14-027 | N-Biph | N-Biph | H | H | H | H | H |
| 2-14-028 | N-Biph | N-Biph | t-bu | H | t-bu | H | H |
| 2-14-029 | N-Biph | N-Biph | methyl | H | methyl | H | H |
| 2-14-030 | N-Biph | N-Biph | t-bu | t-bu | t-bu | t-bu | t-bu |
| 2-14-031 | N-Biph | N-Biph | methyl | methyl | methyl | methyl | methyl |
| 2-14-032 | N-Biph | N-Biph | b1 | H | b1 | H | H |
| 2-14-033 | N-Biph | N-Biph | b2 | H | b2 | H | H |
| 2-14-034 | N-Biph | N-Biph | b3 | H | b3 | H | H |
| 2-14-035 | N-Biph | N-Biph | t-bu | H | t-bu | H | t-bu |
| 2-14-036 | N-Biph | N-Biph | methyl | H | methyl | H | methyl |
| 2-14-037 | N-Biph | N-Biph | b1 | H | b1 | H | b1 |
| 2-14-038 | N-Biph | N-Biph | b2 | H | b2 | H | b2 |
| 2-14-039 | N-Biph | N-Biph | b3 | H | b3 | H | b3 |
| 2-14-040 | N-Ph | O | H | H | H | H | H |
| 2-14-041 | N-Ph | O | t-bu | H | t-bu | H | H |
| 2-14-042 | N-Ph | O | methyl | H | methyl | H | H |
| 2-14-043 | N-Ph | O | t-bu | t-bu | t-bu | t-bu | t-bu |
| 2-14-044 | N-Ph | O | methyl | methyl | methyl | methyl | methyl |
| 2-14-045 | N-Ph | O | b1 | H | b1 | H | H |
| 2-14-046 | N-Ph | O | b2 | H | b2 | H | H |
| 2-14-047 | N-Ph | O | b3 | H | b3 | H | H |
| 2-14-048 | N-Ph | O | t-bu | H | t-bu | H | t-bu |
| 2-14-049 | N-Ph | O | methyl | H | methyl | H | methyl |
| 2-14-050 | N-Ph | O | b1 | H | b1 | H | b1 |
| 2-14-051 | N-Ph | O | b2 | H | b2 | H | b2 |
| 2-14-052 | N-Ph | O | b3 | H | b3 | H | b3 |
| 2-14-053 | N-Ph | S | H | H | H | H | H |
| 2-14-054 | N-Ph | S | t-bu | H | t-bu | H | H |
| 2-14-055 | N-Ph | S | methyl | H | methyl | H | H |
| 2-14-056 | N-Ph | S | t-bu | t-bu | t-bu | t-bu | t-bu |
| 2-14-057 | N-Ph | S | methyl | methyl | methyl | methyl | methyl |
| 2-14-058 | N-Ph | S | b1 | H | b1 | H | H |
| 2-14-059 | N-Ph | S | b2 | H | b2 | H | H |
| 2-14-060 | N-Ph | S | b3 | H | b3 | H | H |
| 2-14-061 | N-Ph | S | t-bu | H | t-bu | H | t-bu |
| 2-14-062 | N-Ph | S | methyl | H | methyl | H | methyl |
| 2-14-063 | N-Ph | S | b1 | H | b1 | H | b1 |
| 2-14-064 | N-Ph | S | b2 | H | b2 | H | b2 |
| 2-14-065 | N-Ph | S | b3 | H | b3 | H | b3 |

Group 2-15
The compounds are represented by the formula 2-15 below, wherein X₁ is B, and Y1, Y2, R1b, R1d, R2a, R2c, R3a are each shown in the table 15 below, 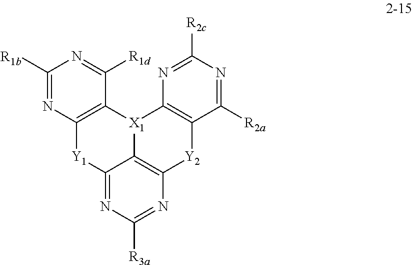
TABLE 15

| Compound | | | | | | | |
|---|---|---|---|---|---|---|---|
| No. | Y1 | Y2 | R1b | R1d | R2a | R2c | R3a |
| 2-15-001 | N-Ph | N-Ph | H | H | H | H | H |
| 2-15-002 | N-Ph | N-Ph | t-bu | H | t-bu | H | H |
| 2-15-003 | N-Ph | N-Ph | methyl | H | methyl | H | H |
| 2-15-004 | N-Ph | N-Ph | t-bu | t-bu | t-bu | t-bu | t-bu |
| 2-15-005 | N-Ph | N-Ph | methyl | methyl | methyl | methyl | methyl |
| 2-15-006 | N-Ph | N-Ph | b1 | H | b1 | b1 | H |
| 2-15-007 | N-Ph | N-Ph | b2 | H | b2 | b2 | H |
| 2-15-008 | N-Ph | N-Ph | b3 | H | b3 | b3 | H |
| 2-15-009 | N-Ph | N-Ph | b1 | H | b1 | b1 | b1 |
| 2-15-010 | N-Ph | N-Ph | b2 | H | b2 | b2 | b2 |
| 2-15-011 | N-Ph | N-Ph | b3 | H | b3 | b3 | b3 |
| 2-15-012 | N-tolyl | N-tolyl | H | H | H | H | H |
| 2-15-013 | N-tolyl | N-tolyl | t-bu | H | t-bu | H | H |
| 2-15-014 | N-tolyl | N-tolyl | methyl | H | methyl | H | H |
| 2-15-015 | N-tolyl | N-tolyl | t-bu | t-bu | t-bu | t-bu | t-bu |
| 2-15-016 | N-tolyl | N-tolyl | methyl | methyl | methyl | methyl | methyl |
| 2-15-017 | N-tolyl | N-tolyl | b1 | H | b1 | b1 | H |
| 2-15-018 | N-tolyl | N-tolyl | b2 | H | b2 | b2 | H |
| 2-15-019 | N-tolyl | N-tolyl | b3 | H | b3 | b3 | H |
| 2-15-020 | N-tolyl | N-tolyl | b1 | H | b1 | b1 | b1 |
| 2-15-021 | N-tolyl | N-tolyl | b2 | H | b2 | b2 | b2 |
| 2-15-022 | N-tolyl | N-tolyl | b3 | H | b3 | b3 | b3 |
| 2-15-023 | N-Biph | N-Biph | H | H | H | H | H |
| 2-15-024 | N-Biph | N-Biph | t-bu | H | t-bu | H | H |
| 2-15-025 | N-Biph | N-Biph | methyl | H | methyl | H | H |
| 2-15-026 | N-Biph | N-Biph | t-bu | t-bu | t-bu | t-bu | t-bu |
| 2-15-027 | N-Biph | N-Biph | methyl | methyl | methyl | methyl | methyl |
| 2-15-028 | N-Biph | N-Biph | b1 | H | b1 | b1 | H |
| 2-15-029 | N-Biph | N-Biph | b2 | H | b2 | b2 | H |
| 2-15-030 | N-Biph | N-Biph | b3 | H | b3 | b3 | H |
| 2-15-031 | N-Bioh | N-Bioh | b1 | H | b1 | b1 | b1 |
| 2-15-032 | N-Biph | N-Biph | b2 | H | b2 | b2 | b2 |
| 2-15-033 | N-Biph | N-Biph | b3 | H | b3 | b3 | b3 |
| 2-15-034 | N-Ph | O | H | H | H | H | H |
| 2-15-035 | N-Ph | O | t-bu | H | t-bu | H | H |
| 2-15-036 | N-Ph | O | methyl | H | methyl | H | H |
| 2-15-037 | N-Ph | O | t-bu | t-bu | t-bu | t-bu | t-bu |
| 2-15-038 | N-Ph | O | methyl | methyl | methyl | methyl | methyl |
| 2-15-039 | N-Ph | O | b1 | H | b1 | b1 | H |
| 2-15-040 | N-Ph | O | b2 | H | b2 | b2 | H |
| 2-15-041 | N-Ph | O | b3 | H | b3 | b3 | H |
| 2-15-042 | N-Ph | O | b1 | H | b1 | b1 | b1 |
| 2-15-043 | N-Ph | O | b2 | H | b2 | b2 | b2 |
| 2-15-044 | N-Ph | O | b3 | H | b3 | b3 | b3 |
| 2-15-045 | N-Ph | S | H | H | H | H | H |
| 2-15-046 | N-Ph | S | t-bu | H | t-bu | H | H |
| 2-15-047 | N-Ph | S | methyl | H | methyl | H | H |
| 2-15-048 | N-Ph | S | t-bu | t-bu | t-bu | t-bu | t-bu |
| 2-15-049 | N-Ph | S | methyl | methyl | methyl | methyl | methyl |
| 2-15-050 | N-Ph | S | b1 | H | b1 | b1 | H |
| 2-15-051 | N-Ph | S | b2 | H | b2 | b2 | H |
| 2-15-052 | N-Ph | S | b3 | H | b3 | b3 | H |
| 2-15-053 | N-Ph | S | b1 | H | b1 | b1 | b1 |
| 2-15-054 | N-Ph | S | b2 | H | b2 | b2 | b2 |
| 2-15-055 | N-Ph | S | b3 | H | b3 | b3 | b3 |

Group 2-16
The compounds are represented by the formula 2-16 below, wherein X₁ is B, and Y1, Y2, R1a, R1c, R2b, R2d, R3a are each shown in the table 16 below, 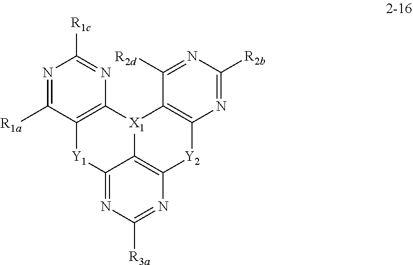
TABLE 16

| Compound | | | | | | | |
|---|---|---|---|---|---|---|---|
| No. | Y1 | Y2 | R1a | R1c | R2b | R2d | R3a |
| 2-16-001 | N-Ph | N-Ph | H | H | H | H | H |
| 2-16-002 | N-Ph | N-Ph | t-bu | H | t-bu | H | H |
| 2-16-003 | N-Ph | N-Ph | methyl | H | methyl | H | H |
| 2-16-004 | N-Ph | N-Ph | t-bu | t-bu | t-bu | t-bu | t-bu |
| 2-16-005 | N-Ph | N-Ph | methyl | methyl | methyl | methyl | methyl |
| 2-16-006 | N-Ph | N-Ph | b1 | b1 | b1 | H | H |
| 2-16-007 | N-Ph | N-Ph | b2 | b2 | b2 | H | H |
| 2-16-008 | N-Ph | N-Ph | b3 | b3 | b3 | H | H |
| 2-16-009 | N-Ph | N-Ph | b1 | b1 | b1 | H | b1 |
| 2-16-010 | N-Ph | N-Ph | b2 | b2 | b2 | H | b2 |
| 2-16-011 | N-Ph | N-Ph | b3 | b3 | b3 | H | b3 |
| 2-16-012 | N-tolyl | N-tolyl | H | H | H | H | H |
| 2-16-013 | N-tolyl | N-tolyl | t-bu | H | t-bu | H | H |
| 2-16-014 | N-tolyl | N-tolyl | methyl | H | methyl | H | H |
| 2-16-015 | N-tolyl | N-tolyl | t-bu | t-bu | t-bu | t-bu | t-bu |
| 2-16-016 | N-tolyl | N-tolyl | methyl | methyl | methyl | methyl | methyl |
| 2-16-017 | N-tolyl | N-tolyl | b1 | b1 | b1 | H | H |
| 2-16-018 | N-tolyl | N-tolyl | b2 | b2 | b2 | H | H |
| 2-16-019 | N-tolyl | N-tolyl | b3 | b3 | b3 | H | H |
| 2-16-020 | N-tolyl | N-tolyl | b1 | b1 | b1 | H | b1 |
| 2-16-021 | N-tolyl | N-tolyl | b2 | b2 | b2 | H | b2 |
| 2-16-022 | N-tolyl | N-tolyl | b3 | b3 | b3 | H | b3 |
| 2-16-023 | N-Biph | N-Biph | H | H | H | H | H |
| 2-16-024 | N-Biph | N-Biph | t-bu | H | t-bu | H | H |
| 2-16-025 | N-Biph | N-Biph | methyl | H | methyl | H | H |
| 2-16-026 | N-Biph | N-Biph | t-bu | t-bu | t-bu | t-bu | t-bu |
| 2-16-027 | N-Biph | N-Biph | methyl | methyl | methyl | methyl | methyl |
| 2-16-028 | N-Biph | N-Biph | b1 | b1 | b1 | H | H |
| 2-16-029 | N-Biph | N-Biph | b2 | b2 | b2 | H | H |
| 2-16-030 | N-Biph | N-Biph | b3 | b3 | b3 | H | H |
| 2-16-031 | N-Biph | N-Biph | b1 | b1 | b1 | H | b1 |
| 2-16-032 | N-Biph | N-Biph | b2 | b2 | b2 | H | b2 |
| 2-16-033 | N-Biph | N-Biph | b3 | b3 | b3 | H | b3 |
| 2-16-034 | N-Ph | O | H | H | H | H | H |
| 2-16-035 | N-Ph | O | t-bu | H | t-bu | H | H |
| 2-16-036 | N-Ph | O | methyl | H | methyl | H | H |
| 2-16-037 | N-Ph | O | t-bu | t-bu | t-bu | t-bu | t-bu |
| 2-16-038 | N-Ph | O | methyl | methyl | methyl | methyl | methyl |
| 2-16-039 | N-Ph | O | b1 | b1 | b1 | H | H |
| 2-16-040 | N-Ph | O | b2 | b2 | b2 | H | H |
| 2-16-041 | N-Ph | O | b3 | b3 | b3 | H | H |
| 2-16-042 | N-Ph | O | b1 | b1 | b1 | H | b1 |
| 2-16-043 | N-Ph | O | b2 | b2 | b2 | H | b2 |
| 2-16-044 | N-Ph | O | b3 | b3 | b3 | H | b3 |
| 2-16-045 | N-Ph | S | H | H | H | H | H |
| 2-16-046 | N-Ph | S | t-bu | H | t-bu | H | H |
| 2-16-047 | N-Ph | S | methyl | H | methyl | H | H |
| 2-16-048 | N-Ph | S | t-bu | t-bu | t-bu | t-bu | t-bu |
| 2-16-049 | N-Ph | S | methyl | methyl | methyl | methyl | methyl |
| 2-16-050 | N-Ph | S | b1 | b1 | b1 | H | H |
| 2-16-051 | N-Ph | S | b2 | b2 | b2 | H | H |
| 2-16-052 | N-Ph | S | b3 | b3 | b3 | H | H |
| 2-16-053 | N-Ph | S | b1 | b1 | b1 | H | b1 |
| 2-16-054 | N-Ph | S | b2 | b2 | b2 | H | b2 |
| 2-16-055 | N-Ph | S | b3 | b3 | b3 | H | b3 |

Group 2-17
The compounds are represented by the formula 2-17 below, wherein X₁ is B, and Y1, Y2, R1a, R1c, R2a, R2c, R3a are each shown in the table 17 below, 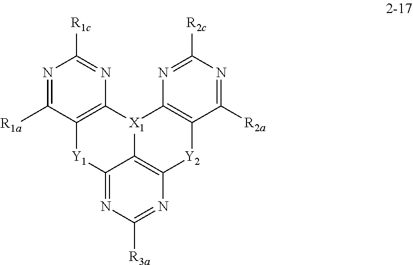
TABLE 17

| Compound | | | | | | | |
|---|---|---|---|---|---|---|---|
| No. | Y1 | Y2 | R1a | R1c | R2a | R2c | R3a |
| 2-17-001 | N-Ph | N-Ph | H | H | H | H | H |
| 2-17-002 | N-Ph | N-Ph | t-bu | H | t-bu | H | H |
| 2-17-003 | N-Ph | N-Ph | methyl | H | methyl | H | H |
| 2-17-004 | N-Ph | N-Ph | t-bu | t-bu | t-bu | t-bu | t-bu |
| 2-17-005 | N-Ph | N-Ph | methyl | methyl | methyl | methyl | methyl |
| 2-17-006 | N-Ph | N-Ph | b1 | H | b1 | H | H |
| 2-17-007 | N-Ph | N-Ph | b2 | H | b2 | H | H |
| 2-17-008 | N-Ph | N-Ph | b3 | H | b3 | H | H |
| 2-17-009 | N-Ph | N-Ph | t-bu | H | t-bu | H | t-bu |
| 2-17-010 | N-Ph | N-Ph | methyl | H | methyl | H | methyl |
| 2-17-011 | N-Ph | N-Ph | b1 | H | b1 | H | b1 |
| 2-17-012 | N-Ph | N-Ph | b2 | H | b2 | H | b2 |
| 2-17-013 | N-Ph | N-Ph | b3 | H | b3 | H | b3 |
| 2-17-014 | N-Ph | N-Ph | b1 | b1 | b1 | b1 | H |
| 2-17-015 | N-Ph | N-Ph | b2 | b2 | b2 | b2 | H |
| 2-17-016 | N-Ph | N-Ph | b3 | b3 | b3 | b3 | H |
| 2-17-017 | N-Ph | N-Ph | b1 | b1 | b1 | b1 | b1 |
| 2-17-018 | N-Ph | N-Ph | b2 | b2 | b2 | b2 | b2 |
| 2-17-019 | N-Ph | N-Ph | b3 | b3 | b3 | b3 | b3 |
| 2-17-020 | N-tolyl | N-tolyl | H | H | H | H | H |
| 2-17-021 | N-tolyl | N-tolyl | t-bu | H | t-bu | H | H |
| 2-17-022 | N-tolyl | N-tolyl | methyl | H | methyl | H | H |
| 2-17-023 | N-tolyl | N-tolyl | t-bu | t-bu | t-bu | t-bu | t-bu |
| 2-17-024 | N-tolyl | N-tolyl | methyl | methyl | methyl | methyl | methyl |
| 2-17-025 | N-tolyl | N-tolyl | b1 | H | b1 | H | H |
| 2-17-026 | N-tolyl | N-tolyl | b2 | H | b2 | H | H |
| 2-17-027 | N-tolyl | N-tolyl | b3 | H | b3 | H | H |
| 2-17-028 | N-tolyl | N-tolyl | t-bu | H | t-bu | H | t-bu |
| 2-17-029 | N-tolyl | N-tolyl | methyl | H | methyl | H | methyl |
| 2-17-030 | N-tolyl | N-tolyl | b1 | H | b1 | H | b1 |
| 2-17-031 | N-tolyl | N-tolyl | b2 | H | b2 | H | b2 |
| 2-17-032 | N-tolyl | N-tolyl | b3 | H | b3 | H | b3 |
| 2-17-033 | N-tolyl | N-tolyl | b1 | b1 | b1 | b1 | H |
| 2-17-034 | N-tolyl | N-tolyl | b2 | b2 | b2 | b2 | H |
| 2-17-035 | N-tolyl | N-tolyl | b3 | b3 | b3 | b3 | H |
| 2-17-036 | N-tolyl | N-tolyl | b1 | b1 | b1 | b1 | b1 |
| 2-17-037 | N-tolyl | N-tolyl | b2 | b2 | b2 | b2 | b2 |
| 2-17-038 | N-tolyl | N-tolyl | b3 | b3 | b3 | b3 | b3 |
| 2-17-039 | N-Biph | N-Biph | H | H | H | H | H |
| 2-17-040 | N-Biph | N-Biph | t-bu | H | t-bu | H | H |
| 2-17-041 | N-Biph | N-Biph | methyl | H | methyl | H | H |
| 2-17-042 | N-Biph | N-Biph | t-bu | t-bu | t-bu | t-bu | t-bu |
| 2-17-043 | N-Biph | N-Biph | methyl | methyl | methyl | methyl | methyl |
| 2-17-044 | N-Biph | N-Biph | b1 | H | b1 | H | H |
| 2-17-045 | N-Biph | N-Biph | b2 | H | b2 | H | H |
| 2-17-046 | N-Biph | N-Biph | b3 | H | b3 | H | H |
| 2-17-047 | N-Biph | N-Biph | t-bu | H | t-bu | H | t-bu |
| 2-17-048 | N-Biph | N-Biph | methyl | H | methyl | H | methyl |
| 2-17-049 | N-Biph | N-Biph | b1 | H | b1 | H | b1 |
| 2-17-050 | N-Biph | N-Biph | b2 | H | b2 | H | b2 |
| 2-17-051 | N-Biph | N-Biph | b3 | H | b3 | H | b3 |
| 2-17-052 | N-Biph | N-Biph | b1 | b1 | b1 | b1 | H |
| 2-17-053 | N-Biph | N-Biph | b2 | b2 | b2 | b2 | H |
| 2-17-054 | N-Biph | N-Biph | b3 | b3 | b3 | b3 | H |
| 2-17-055 | N-Biph | N-Biph | b1 | b1 | b1 | b1 | b1 |
| 2-17-056 | N-Biph | N-Biph | b2 | b2 | b2 | b2 | b2 |
| 2-17-057 | N-Biph | N-Biph | b3 | b3 | b3 | b3 | b3 |
| 2-17-058 | N-Ph | O | H | H | H | H | H |
| 2-17-059 | N-Ph | O | t-bu | H | t-bu | H | H |
| 2-17-060 | N-Ph | O | methyl | H | methyl | H | H |
| 2-17-061 | N-Ph | O | t-bu | t-bu | t-bu | t-bu | t-bu |
| 2-17-062 | N-Ph | O | methyl | methyl | methyl | methyl | methyl |
| 2-17-063 | N-Ph | O | b1 | H | b1 | H | H |
| 2-17-064 | N-Ph | O | b2 | H | b2 | H | H |
| 2-17-065 | N-Ph | O | b3 | H | b3 | H | H |
| 2-17-066 | N-Ph | O | t-bu | H | t-bu | H | t-bu |
| 2-17-067 | N-Ph | O | methyl | H | methyl | H | methyl |
| 2-17-068 | N-Ph | O | b1 | H | b1 | H | b1 |
| 2-17-069 | N-Ph | O | b2 | H | b2 | H | b2 |
| 2-17-070 | N-Ph | O | b3 | H | b3 | H | b3 |
| 2-17-071 | N-Ph | O | b1 | b1 | b1 | b1 | H |
| 2-17-072 | N-Ph | O | b2 | b2 | b2 | b2 | H |
| 2-17-073 | N-Ph | O | b3 | b3 | b3 | b3 | H |
| 2-17-074 | N-Ph | O | b1 | b1 | b1 | b1 | b1 |
| 2-17-075 | N-Ph | O | b2 | b2 | b2 | b2 | b2 |
| 2-17-076 | N-Ph | O | b3 | b3 | b3 | b3 | b3 |
| 2-17-077 | N-Ph | S | H | H | H | H | H |
| 2-17-078 | N-Ph | S | t-bu | H | t-bu | H | H |
| 2-17-079 | N-Ph | S | methyl | H | methyl | H | H |
| 2-17-080 | N-Ph | S | t-bu | t-bu | t-bu | t-bu | t-bu |
| 2-17-081 | N-Ph | S | methyl | methyl | methyl | methyl | methyl |
| 2-17-082 | N-Ph | S | b1 | H | b1 | H | H |
| 2-17-083 | N-Ph | S | b2 | H | b2 | H | H |
| 2-17-084 | N-Ph | S | b3 | H | b3 | H | H |
| 2-17-085 | N-Ph | S | t-bu | H | t-bu | H | t-bu |
| 2-17-086 | N-Ph | S | methyl | H | methyl | H | methyl |
| 2-17-087 | N-Ph | S | b1 | H | b1 | H | b1 |
| 2-17-088 | N-Ph | S | b2 | H | b2 | H | b2 |
| 2-17-089 | N-Ph | S | b3 | H | b3 | H | b3 |
| 2-17-090 | N-Ph | S | b1 | b1 | b1 | b1 | H |
| 2-17-091 | N-Ph | S | b2 | b2 | b2 | b2 | H |
| 2-17-092 | N-Ph | S | b3 | b3 | b3 | b3 | H |
| 2-17-093 | N-Ph | S | b1 | b1 | b1 | b1 | b1 |
| 2-17-094 | N-Ph | S | b2 | b2 | b2 | b2 | b2 |
| 2-17-095 | N-Ph | S | b3 | b3 | b3 | b3 | b3 |

Group 2-18
The compounds are represented by the formula 2-18 below, wherein CY₂ and CY₃ are each a benzene group, X₁ is B, and Y1, Y2, Y3, R1b, (R2)a2, (R3)a3 are each shown in the table 18 below, 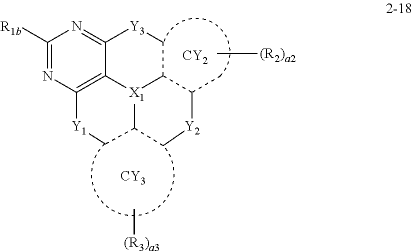
TABLE 18

| Compound | | | | | |
|---|---|---|---|---|---|
| No. | Y1/Y3 | Y2 | R1b | (R2)a2 | (R3)a3 |
| 2-18-001 | N-Ph | N-Ph | H | H | H |
| 2-18-002 | N-Ph | N-Ph | t-bu | H | H |
| 2-18-003 | N-Ph | N-Ph | methyl | H | H |
| 2-18-004 | N-Ph | N-Ph | t-bu | t-bu | t-bu |
| 2-18-005 | N-Ph | N-Ph | methyl | methyl | methyl |
| 2-18-006 | N-Ph | N-Ph | b1 | H | H |
| 2-18-007 | N-Ph | N-Ph | b2 | H | H |
| 2-18-008 | N-Ph | N-Ph | b3 | H | H |
| 2-18-009 | N-Ph | N-Ph | H | H | a1 |
| 2-18-010 | N-Ph | N-Ph | t-bu | H | a1 |
| 2-18-011 | N-Ph | N-Ph | methyl | H | a1 |
| 2-18-012 | N-Ph | N-Ph | b1 | H | a1 |
| 2-18-013 | N-Ph | N-Ph | b2 | H | a1 |
| 2-18-014 | N-Ph | N-Ph | b3 | H | a1 |
| 2-18-015 | N-Ph | N-Ph | H | H | a2 |
| 2-18-016 | N-Ph | N-Ph | t-bu | H | a2 |
| 2-18-017 | N-Ph | N-Ph | methyl | H | a2 |
| 2-18-018 | N-Ph | N-Ph | b1 | H | a2 |
| 2-18-019 | N-Ph | N-Ph | b2 | H | a2 |
| 2-18-020 | N-Ph | N-Ph | b3 | H | a2 |
| 2-18-021 | N-Ph | N-Ph | H | H | a3 |
| 2-18-022 | N-Ph | N-Ph | t-bu | H | a3 |
| 2-18-023 | N-Ph | N-Ph | methyl | H | a3 |
| 2-18-024 | N-Ph | N-Ph | b1 | H | a3 |
| 2-18-025 | N-Ph | N-Ph | b2 | H | a3 |
| 2-18-026 | N-Ph | N-Ph | b3 | H | a3 |
| 2-18-027 | N-Ph | N-Ph | H | H | a4 |
| 2-18-028 | N-Ph | N-Ph | t-bu | H | a4 |
| 2-18-029 | N-Ph | N-Ph | methyl | H | a4 |
| 2-18-030 | N-Ph | N-Ph | b1 | H | a4 |
| 2-18-031 | N-Ph | N-Ph | b2 | H | a4 |
| 2-18-032 | N-Ph | N-Ph | b3 | H | a4 |
| 2-18-033 | N-Ph | N-Ph | H | H | a5 |
| 2-18-034 | N-Ph | N-Ph | t-bu | H | a5 |
| 2-18-035 | N-Ph | N-Ph | methyl | H | a5 |
| 2-18-036 | N-Ph | N-Ph | b1 | H | a5 |
| 2-18-037 | N-Ph | N-Ph | b2 | H | a5 |
| 2-18-038 | N-Ph | N-Ph | b3 | H | a5 |
| 2-18-039 | N-Ph | N-Ph | H | H | a6 |
| 2-18-040 | N-Ph | N-Ph | t-bu | H | a6 |
| 2-18-041 | N-Ph | N-Ph | methyl | H | a6 |
| 2-18-042 | N-Ph | N-Ph | b1 | H | a6 |
| 2-18-043 | N-Ph | N-Ph | b2 | H | a6 |
| 2-18-044 | N-Ph | N-Ph | b3 | H | a6 |
| 2-18-045 | N-Ph | N-Ph | H | H | b1 |
| 2-18-046 | N-Ph | N-Ph | t-bu | H | b1 |
| 2-18-047 | N-Ph | N-Ph | methyl | H | b1 |
| 2-18-048 | N-Ph | N-Ph | b1 | H | b1 |
| 2-18-049 | N-Ph | N-Ph | b2 | H | b1 |
| 2-18-050 | N-Ph | N-Ph | b3 | H | b1 |
| 2-18-051 | N-Ph | N-Ph | H | H | b2 |
| 2-18-052 | N-Ph | N-Ph | t-bu | H | b2 |
| 2-18-053 | N-Ph | N-Ph | methyl | H | b2 |
| 2-18-054 | N-Ph | N-Ph | b1 | H | b2 |
| 2-18-055 | N-Ph | N-Ph | b2 | H | b2 |
| 2-18-056 | N-Ph | N-Ph | b3 | H | b2 |
| 2-18-057 | N-Ph | N-Ph | H | H | b3 |
| 2-18-058 | N-Ph | N-Ph | t-bu | H | b3 |
| 2-18-059 | N-Ph | N-Ph | methyl | H | b3 |
| 2-18-060 | N-Ph | N-Ph | b1 | H | b3 |
| 2-18-061 | N-Ph | N-Ph | b2 | H | b3 |
| 2-18-062 | N-Ph | N-Ph | b3 | H | b3 |
| 2-18-063 | N-Ph | N-Ph | H | H | b4 |
| 2-18-064 | N-Ph | N-Ph | t-bu | H | b4 |
| 2-18-065 | N-Ph | N-Ph | methyl | H | b4 |
| 2-18-066 | N-Ph | N-Ph | b1 | H | b4 |
| 2-18-067 | N-Ph | N-Ph | b2 | H | b4 |
| 2-18-068 | N-Ph | N-Ph | b3 | H | b4 |
| 2-18-069 | N-Ph | N-Ph | H | H | b5 |
| 2-18-070 | N-Ph | N-Ph | t-bu | H | b5 |
| 2-18-071 | N-Ph | N-Ph | methyl | H | b5 |
| 2-18-072 | N-Ph | N-Ph | b1 | H | b5 |
| 2-18-073 | N-Ph | N-Ph | b2 | H | b5 |
| 2-18-074 | N-Ph | N-Ph | b3 | H | b5 |
| 2-18-075 | N-Ph | N-Ph | H | H | b6 |
| 2-18-076 | N-Ph | N-Ph | t-bu | H | b6 |
| 2-18-077 | N-Ph | N-Ph | methyl | H | b6 |
| 2-18-078 | N-Ph | N-Ph | b1 | H | b6 |
| 2-18-079 | N-Ph | N-Ph | b2 | H | b6 |
| 2-18-080 | N-Ph | N-Ph | b3 | H | b6 |
| 2-18-081 | N-Ph | N-Ph | H | H | b7 |
| 2-18-082 | N-Ph | N-Ph | t-bu | H | b7 |
| 2-18-083 | N-Ph | N-Ph | methyl | H | b7 |
| 2-18-084 | N-Ph | N-Ph | b1 | H | b7 |
| 2-18-085 | N-Ph | N-Ph | b2 | H | b7 |
| 2-18-086 | N-Ph | N-Ph | b3 | H | b7 |
| 2-18-087 | N-Ph | N-Ph | H | H | b8 |
| 2-18-088 | N-Ph | N-Ph | t-bu | H | b8 |
| 2-18-089 | N-Ph | N-Ph | methyl | H | b8 |
| 2-18-090 | N-Ph | N-Ph | b1 | H | b8 |
| 2-18-091 | N-Ph | N-Ph | b2 | H | b8 |
| 2-18-092 | N-Ph | N-Ph | b3 | H | b8 |
| 2-18-093 | N-Ph | N-Ph | H | H | b9 |
| 2-18-094 | N-Ph | N-Ph | t-bu | H | b9 |
| 2-18-095 | N-Ph | N-Ph | methyl | H | b9 |
| 2-18-096 | N-Ph | N-Ph | b1 | H | b9 |
| 2-18-097 | N-Ph | N-Ph | b2 | H | b9 |
| 2-18-098 | N-Ph | N-Ph | b3 | H | b9 |
| 2-18-099 | N-Ph | N-Ph | H | H | b1 0 |
| 2-18-100 | N-Ph | N-Ph | t-bu | H | b1 0 |
| 2-18-101 | N-Ph | N-Ph | methyl | H | b1 0 |
| 2-18-102 | N-Ph | N-Ph | b1 | H | b1 0 |
| 2-18-103 | N-Ph | N-Ph | b2 | H | b1 0 |
| 2-18-104 | N-Ph | N-Ph | b3 | H | b1 0 |
| 2-18-105 | N-Ph | N-Ph | H | a1 | H |
| 2-18-106 | N-Ph | N-Ph | t-bu | a1 | H |
| 2-18-107 | N-Ph | N-Ph | methyl | a1 | H |
| 2-18-108 | N-Ph | N-Ph | b1 | a1 | H |
| 2-18-109 | N-Ph | N-Ph | b2 | a1 | H |
| 2-18-110 | N-Ph | N-Ph | b3 | a1 | H |
| 2-18-111 | N-Ph | N-Ph | H | a2 | H |
| 2-18-112 | N-Ph | N-Ph | t-bu | a2 | H |
| 2-18-113 | N-Ph | N-Ph | methyl | a2 | H |
| 2-18-114 | N-Ph | N-Ph | b1 | a2 | H |
| 2-18-115 | N-Ph | N-Ph | b2 | a2 | H |
| 2-18-116 | N-Ph | N-Ph | b3 | a2 | H |
| 2-18-117 | N-Ph | N-Ph | H | a3 | H |
| 2-18-118 | N-Ph | N-Ph | t-bu | a3 | H |
| 2-18-119 | N-Ph | N-Ph | methyl | a3 | H |
| 2-18-120 | N-Ph | N-Ph | b1 | a3 | H |
| 2-18-121 | N-Ph | N-Ph | b2 | a3 | H |
| 2-18-122 | N-Ph | N-Ph | b3 | a3 | H |
| 2-18-123 | N-Ph | N-Ph | H | a4 | H |
| 2-18-124 | N-Ph | N-Ph | t-bu | a4 | H |
| 2-18-125 | N-Ph | N-Ph | methyl | a4 | H |
| 2-18-126 | N-Ph | N-Ph | b1 | a4 | H |
| 2-18-127 | N-Ph | N-Ph | b2 | a4 | H |
| 2-18-128 | N-Ph | N-Ph | b3 | a4 | H |
| 2-18-129 | N-Ph | N-Ph | H | a5 | H |
| 2-18-130 | N-Ph | N-Ph | t-bu | a5 | H |
| 2-18-131 | N-Ph | N-Ph | methyl | a5 | H |
| 2-18-132 | N-Ph | N-Ph | b1 | a5 | H |
| 2-18-133 | N-Ph | N-Ph | b2 | a5 | H |
| 2-18-134 | N-Ph | N-Ph | b3 | a5 | H |
| 2-18-135 | N-Ph | N-Ph | H | a6 | H |
| 2-18-136 | N-Ph | N-Ph | t-bu | a6 | H |
| 2-18-137 | N-Ph | N-Ph | methyl | a6 | H |
| 2-18-138 | N-Ph | N-Ph | b1 | a6 | H |
| 2-18-139 | N-Ph | N-Ph | b2 | a6 | H |
| 2-18-140 | N-Ph | N-Ph | b3 | a6 | H |
| 2-18-141 | N-Ph | N-Ph | H | b1 | H |
| 2-18-142 | N-Ph | N-Ph | t-bu | b1 | H |
| 2-18-143 | N-Ph | N-Ph | methyl | b1 | H |
| 2-18-144 | N-Ph | N-Ph | b1 | b1 | H |
| 2-18-145 | N-Ph | N-Ph | b2 | b1 | H |
| 2-18-146 | N-Ph | N-Ph | b3 | b1 | H |
| 2-18-147 | N-Ph | N-Ph | H | b2 | H |
| 2-18-148 | N-Ph | N-Ph | t-bu | b2 | H |
| 2-18-149 | N-Ph | N-Ph | methyl | b2 | H |
| 2-18-150 | N-Ph | N-Ph | b1 | b2 | H |
| 2-18-151 | N-Ph | N-Ph | b2 | b2 | H |
| 2-18-152 | N-Ph | N-Ph | b3 | b2 | H |
| 2-18-153 | N-Ph | N-Ph | H | b3 | H |
| 2-18-154 | N-Ph | N-Ph | t-bu | b3 | H |
| 2-18-155 | N-Ph | N-Ph | methyl | b3 | H |
| 2-18-156 | N-Ph | N-Ph | b1 | b3 | H |
| 2-18-157 | N-Ph | N-Ph | b2 | b3 | H |
| 2-18-158 | N-Ph | N-Ph | b3 | b3 | H |
| 2-18-159 | N-Ph | N-Ph | H | b4 | H |
| 2-18-160 | N-Ph | N-Ph | t-bu | b4 | H |
| 2-18-161 | N-Ph | N-Ph | methyl | b4 | H |
| 2-18-162 | N-Ph | N-Ph | b1 | b4 | H |
| 2-18-163 | N-Ph | N-Ph | b2 | b4 | H |
| 2-18-164 | N-Ph | N-Ph | b3 | b4 | H |
| 2-18-165 | N-Ph | N-Ph | H | b5 | H |
| 2-18-166 | N-Ph | N-Ph | t-bu | b5 | H |
| 2-18-167 | N-Ph | N-Ph | methyl | b5 | H |
| 2-18-168 | N-Ph | N-Ph | b1 | b5 | H |
| 2-18-169 | N-Ph | N-Ph | b2 | b5 | H |
| 2-18-170 | N-Ph | N-Ph | b3 | b5 | H |
| 2-18-171 | N-Ph | N-Ph | H | b6 | H |
| 2-18-172 | N-Ph | N-Ph | t-bu | b6 | H |
| 2-18-173 | N-Ph | N-Ph | methyl | b6 | H |
| 2-18-174 | N-Ph | N-Ph | b1 | b6 | H |
| 2-18-175 | N-Ph | N-Ph | b2 | b6 | H |
| 2-18-176 | N-Ph | N-Ph | b3 | b6 | H |
| 2-18-177 | N-Ph | N-Ph | H | b7 | H |
| 2-18-178 | N-Ph | N-Ph | t-bu | b7 | H |
| 2-18-179 | N-Ph | N-Ph | methyl | b7 | H |
| 2-18-180 | N-Ph | N-Ph | b1 | b7 | H |
| 2-18-181 | N-Ph | N-Ph | b2 | b7 | H |
| 2-18-182 | N-Ph | N-Ph | b3 | b7 | H |
| 2-18-183 | N-Ph | N-Ph | H | b8 | H |
| 2-18-184 | N-Ph | N-Ph | t-bu | b8 | H |
| 2-18-185 | N-Ph | N-Ph | methyl | b8 | H |
| 2-18-186 | N-Ph | N-Ph | b1 | b8 | H |
| 2-18-187 | N-Ph | N-Ph | b2 | b8 | H |
| 2-18-188 | N-Ph | N-Ph | b3 | b8 | H |
| 2-18-189 | N-Ph | N-Ph | H | b9 | H |
| 2-18-190 | N-Ph | N-Ph | t-bu | b9 | H |
| 2-18-191 | N-Ph | N-Ph | methyl | b9 | H |
| 2-18-192 | N-Ph | N-Ph | b1 | b9 | H |
| 2-18-193 | N-Ph | N-Ph | b2 | b9 | H |
| 2-18-194 | N-Ph | N-Ph | b3 | b9 | H |
| 2-18-195 | N-Ph | N-Ph | H | b10 | H |
| 2-18-196 | N-Ph | N-Ph | t-bu | b10 | H |
| 2-18-197 | N-Ph | N-Ph | methyl | b10 | H |
| 2-18-198 | N-Ph | N-Ph | b1 | b10 | H |
| 2-18-199 | N-Ph | N-Ph | b2 | b10 | H |
| 2-18-200 | N-Ph | N-Ph | b3 | b10 | H |
| 2-18-201 | N-Ph | N-Ph | H | a1 | a1 |
| 2-18-202 | N-Ph | N-Ph | t-bu | a1 | a1 |
| 2-18-203 | N-Ph | N-Ph | methyl | a1 | a1 |
| 2-18-204 | N-Ph | N-Ph | b1 | a1 | a1 |
| 2-18-205 | N-Ph | N-Ph | b2 | a1 | a1 |
| 2-18-206 | N-Ph | N-Ph | b3 | a1 | a1 |
| 2-18-207 | N-Ph | N-Ph | H | a2 | a2 |
| 2-18-208 | N-Ph | N-Ph | t-bu | a2 | a2 |
| 2-18-209 | N-Ph | N-Ph | methyl | a2 | a2 |
| 2-18-210 | N-Ph | N-Ph | b1 | a2 | a2 |
| 2-18-211 | N-Ph | N-Ph | b2 | a2 | a2 |
| 2-18-212 | N-Ph | N-Ph | b3 | a2 | a2 |
| 2-18-213 | N-Ph | N-Ph | H | a3 | a3 |
| 2-18-214 | N-Ph | N-Ph | t-bu | a3 | a3 |
| 2-18-215 | N-Ph | N-Ph | methyl | a3 | a3 |
| 2-18-216 | N-Ph | N-Ph | b1 | a3 | a3 |
| 2-18-217 | N-Ph | N-Ph | b2 | a3 | a3 |
| 2-18-218 | N-Ph | N-Ph | b3 | a3 | a3 |
| 2-18-219 | N-Ph | N-Ph | H | b1 | b1 |
| 2-18-220 | N-Ph | N-Ph | t-bu | b1 | b1 |
| 2-18-221 | N-Ph | N-Ph | methyl | b1 | b1 |
| 2-18-222 | N-Ph | N-Ph | b1 | b1 | b1 |
| 2-18-223 | N-Ph | N-Ph | b2 | b1 | b1 |
| 2-18-224 | N-Ph | N-Ph | b3 | b1 | b1 |
| 2-18-225 | N-Ph | N-Ph | H | b2 | b2 |
| 2-18-226 | N-Ph | N-Ph | t-bu | b2 | b2 |
| 2-18-227 | N-Ph | N-Ph | methyl | b2 | b2 |
| 2-18-228 | N-Ph | N-Ph | b1 | b2 | b2 |
| 2-18-229 | N-Ph | N-Ph | b2 | b2 | b2 |
| 2-18-230 | N-Ph | N-Ph | b3 | b2 | b2 |
| 2-18-231 | N-Ph | N-Ph | H | b3 | b3 |
| 2-18-232 | N-Ph | N-Ph | t-bu | b3 | b3 |
| 2-18-233 | N-Ph | N-Ph | methyl | b3 | b3 |
| 2-18-234 | N-Ph | N-Ph | b1 | b3 | b3 |
| 2-18-235 | N-Ph | N-Ph | b2 | b3 | b3 |
| 2-18-236 | N-Ph | N-Ph | b3 | b3 | b3 |
| 2-18-237 | N-Ph | N-Ph | H | b5 | b5 |
| 2-18-238 | N-Ph | N-Ph | t-bu | b5 | b5 |
| 2-18-239 | N-Ph | N-Ph | methyl | b5 | b5 |
| 2-18-240 | N-Ph | N-Ph | b1 | b5 | b5 |
| 2-18-241 | N-Ph | N-Ph | b2 | b5 | b5 |
| 2-18-242 | N-Ph | N-Ph | b3 | b5 | b5 |
| 2-18-243 | N-Ph | N-Ph | H | b6 | b6 |
| 2-18-244 | N-Ph | N-Ph | t-bu | b6 | b6 |
| 2-18-245 | N-Ph | N-Ph | methyl | b6 | b6 |
| 2-18-246 | N-Ph | N-Ph | b1 | b6 | b6 |
| 2-18-247 | N-Ph | N-Ph | b2 | b6 | b6 |
| 2-18-248 | N-Ph | N-Ph | b3 | b6 | b6 |
| 2-18-249 | N-Ph | N-Ph | H | b7 | b7 |
| 2-18-250 | N-Ph | N-Ph | t-bu | b7 | b7 |
| 2-18-251 | N-Ph | N-Ph | methyl | b7 | b7 |
| 2-18-252 | N-Ph | N-Ph | b1 | b7 | b7 |
| 2-18-253 | N-Ph | N-Ph | b2 | b7 | b7 |
| 2-18-254 | N-Ph | N-Ph | b3 | b7 | b7 |
| 2-18-255 | N-tolyl | N-tolyl | H | H | H |
| 2-18-256 | N-tolyl | N-tolyl | t-bu | H | H |
| 2-18-257 | N-tolyl | N-tolyl | methyl | H | H |
| 2-18-258 | N-tolyl | N-tolyl | t-bu | t-bu | t-bu |
| 2-18-259 | N-tolyl | N-tolyl | methyl | methyl | methyl |
| 2-18-260 | N-tolyl | N-tolyl | b1 | H | H |
| 2-18-261 | N-tolyl | N-tolyl | b2 | H | H |
| 2-18-262 | N-tolyl | N-tolyl | b3 | H | H |
| 2-18-263 | N-tolyl | N-tolyl | H | H | a1 |
| 2-18-264 | N-tolyl | N-tolyl | t-bu | H | a1 |
| 2-18-265 | N-tolyl | N-tolyl | methyl | H | a1 |
| 2-18-266 | N-tolyl | N-tolyl | b1 | H | a1 |
| 2-18-267 | N-tolyl | N-tolyl | b2 | H | a1 |
| 2-18-268 | N-tolyl | N-tolyl | b3 | H | a1 |
| 2-18-269 | N-tolyl | N-tolyl | H | H | a2 |
| 2-18-270 | N-tolyl | N-tolyl | t-bu | H | a2 |
| 2-18-271 | N-tolyl | N-tolyl | methyl | H | a2 |
| 2-18-272 | N-tolyl | N-tolyl | b1 | H | a2 |
| 2-18-273 | N-tolyl | N-tolyl | b2 | H | a2 |
| 2-18-274 | N-tolyl | N-tolyl | b3 | H | a2 |
| 2-18-275 | N-tolyl | N-tolyl | H | H | a3 |
| 2-18-276 | N-tolyl | N-tolyl | t-bu | H | a3 |
| 2-18-277 | N-tolyl | N-tolyl | methyl | H | a3 |
| 2-18-278 | N-tolyl | N-tolyl | b1 | H | a3 |
| 2-18-279 | N-tolyl | N-tolyl | b2 | H | a3 |
| 2-18-280 | N-tolyl | N-tolyl | b3 | H | a3 |
| 2-18-281 | N-tolyl | N-tolyl | H | H | a4 |
| 2-18-282 | N-tolyl | N-tolyl | t-bu | H | a4 |
| 2-18-283 | N-tolyl | N-tolyl | methyl | H | a4 |
| 2-18-284 | N-tolyl | N-tolyl | b1 | H | a4 |
| 2-18-285 | N-tolyl | N-tolyl | b2 | H | a4 |
| 2-18-286 | N-tolyl | N-tolyl | b3 | H | a4 |
| 2-18-287 | N-tolyl | N-tolyl | H | H | a5 |
| 2-18-288 | N-tolyl | N-tolyl | t-bu | H | a5 |
| 2-18-289 | N-tolyl | N-tolyl | methyl | H | a5 |
| 2-18-290 | N-tolyl | N-tolyl | b1 | H | a5 |
| 2-18-291 | N-tolyl | N-tolyl | b2 | H | a5 |
| 2-18-292 | N-tolyl | N-tolyl | b3 | H | a5 |
| 2-18-293 | N-tolyl | N-tolyl | H | H | a6 |
| 2-18-294 | N-tolyl | N-tolyl | t-bu | H | a6 |
| 2-18-295 | N-tolyl | N-tolyl | methyl | H | a6 |
| 2-18-296 | N-tolyl | N-tolyl | b1 | H | a6 |
| 2-18-297 | N-tolyl | N-tolyl | b2 | H | a6 |
| 2-18-298 | N-tolyl | N-tolyl | b3 | H | a6 |
| 2-18-299 | N-tolyl | N-tolyl | H | H | b1 |
| 2-18-300 | N-tolyl | N-tolyl | t-bu | H | b1 |
| 2-18-301 | N-tolyl | N-tolyl | methyl | H | b1 |
| 2-18-302 | N-tolyl | N-tolyl | b1 | H | b1 |
| 2-18-303 | N-tolyl | N-tolyl | b2 | H | b1 |
| 2-18-304 | N-tolyl | N-tolyl | b3 | H | b1 |
| 2-18-305 | N-tolyl | N-tolyl | H | H | b2 |
| 2-18-306 | N-tolyl | N-tolyl | t-bu | H | b2 |
| 2-18-307 | N-tolyl | N-tolyl | methyl | H | b2 |
| 2-18-308 | N-tolyl | N-tolyl | b1 | H | b2 |
| 2-18-309 | N-tolyl | N-tolyl | b2 | H | b2 |
| 2-18-310 | N-tolyl | N-tolyl | b3 | H | b2 |
| 2-18-311 | N-tolyl | N-tolyl | H | H | b3 |
| 2-18-312 | N-tolyl | N-tolyl | t-bu | H | b3 |
| 2-18-313 | N-tolyl | N-tolyl | methyl | H | b3 |
| 2-18-314 | N-tolyl | N-tolyl | b1 | H | b3 |
| 2-18-315 | N-tolyl | N-tolyl | b2 | H | b3 |
| 2-18-316 | N-tolyl | N-tolyl | b3 | H | b3 |
| 2-18-317 | N-tolyl | N-tolyl | H | H | b4 |
| 2-18-318 | N-tolyl | N-tolyl | t-bu | H | b4 |
| 2-18-319 | N-tolyl | N-tolyl | methyl | H | b4 |
| 2-18-320 | N-tolyl | N-tolyl | b1 | H | b4 |
| 2-18-321 | N-tolyl | N-tolyl | b2 | H | b4 |
| 2-18-322 | N-tolyl | N-tolyl | b3 | H | b4 |
| 2-18-323 | N-tolyl | N-tolyl | H | H | b5 |
| 2-18-324 | N-tolyl | N-tolyl | t-bu | H | b5 |
| 2-18-325 | N-tolyl | N-tolyl | methyl | H | b5 |
| 2-18-326 | N-tolyl | N-tolyl | b1 | H | b5 |
| 2-18-327 | N-tolyl | N-tolyl | b2 | H | b5 |
| 2-18-328 | N-tolyl | N-tolyl | b3 | H | b5 |
| 2-18-329 | N-tolyl | N-tolyl | H | H | b6 |
| 2-18-330 | N-tolyl | N-tolyl | t-bu | H | b6 |
| 2-18-331 | N-tolyl | N-tolyl | methyl | H | b6 |
| 2-18-332 | N-tolyl | N-tolyl | b1 | H | b6 |
| 2-18-333 | N-tolyl | N-tolyl | b2 | H | b6 |
| 2-18-334 | N-tolyl | N-tolyl | b3 | H | b6 |
| 2-18-335 | N-tolyl | N-tolyl | H | H | b7 |
| 2-18-336 | N-tolyl | N-tolyl | t-bu | H | b7 |
| 2-18-337 | N-tolyl | N-tolyl | methyl | H | b7 |
| 2-18-338 | N-tolyl | N-tolyl | b1 | H | b7 |
| 2-18-339 | N-tolyl | N-tolyl | b2 | H | b7 |
| 2-18-340 | N-tolyl | N-tolyl | b3 | H | b7 |
| 2-18-341 | N-tolyl | N-tolyl | H | H | b8 |
| 2-18-342 | N-tolyl | N-tolyl | t-bu | H | b8 |
| 2-18-343 | N-tolyl | N-tolyl | methyl | H | b8 |
| 2-18-344 | N-tolyl | N-tolyl | b1 | H | b8 |
| 2-18-345 | N-tolyl | N-tolyl | b2 | H | b8 |
| 2-18-346 | N-tolyl | N-tolyl | b3 | H | b8 |
| 2-18-347 | N-tolyl | N-tolyl | H | H | b9 |
| 2-18-348 | N-tolyl | N-tolyl | t-bu | H | b9 |
| 2-18-349 | N-tolyl | N-tolyl | methyl | H | b9 |
| 2-18-350 | N-tolyl | N-tolyl | b1 | H | b9 |
| 2-18-351 | N-tolyl | N-tolyl | b2 | H | b9 |
| 2-18-352 | N-tolyl | N-tolyl | b3 | H | b9 |
| 2-18-353 | N-tolyl | N-tolyl | H | H | b10 |
| 2-18-354 | N-tolyl | N-tolyl | t-bu | H | b10 |
| 2-18-355 | N-tolyl | N-tolyl | methyl | H | b1 0 |
| 2-18-356 | N-tolyl | N-tolyl | b1 | H | b1 0 |
| 2-18-357 | N-tolyl | N-tolyl | b2 | H | b1 0 |
| 2-18-358 | N-tolyl | N-tolyl | b3 | H | b1 0 |
| 2-18-359 | N-tolyl | N-tolyl | H | a1 | H |
| 2-18-360 | N-tolyl | N-tolyl | t-bu | a1 | H |
| 2-18-361 | N-tolyl | N-tolyl | methyl | a1 | H |
| 2-18-362 | N-tolyl | N-tolyl | b1 | a1 | H |
| 2-18-363 | N-tolyl | N-tolyl | b2 | a1 | H |
| 2-18-364 | N-tolyl | N-tolyl | b3 | a1 | H |
| 2-18-365 | N-tolyl | N-tolyl | H | a2 | H |
| 2-18-366 | N-tolyl | N-tolyl | t-bu | a2 | H |
| 2-18-367 | N-tolyl | N-tolyl | methyl | a2 | H |
| 2-18-368 | N-tolyl | N-tolyl | b1 | a2 | H |
| 2-18-369 | N-tolyl | N-tolyl | b2 | a2 | H |
| 2-18-370 | N-tolyl | N-tolyl | b3 | a2 | H |
| 2-18-371 | N-tolyl | N-tolyl | H | a3 | H |
| 2-18-372 | N-tolyl | N-tolyl | t-bu | a3 | H |
| 2-18-373 | N-tolyl | N-tolyl | methyl | a3 | H |
| 2-18-374 | N-tolyl | N-tolyl | b1 | a3 | H |
| 2-18-375 | N-tolyl | N-tolyl | b2 | a3 | H |
| 2-18-376 | N-tolyl | N-tolyl | b3 | a3 | H |
| 2-18-377 | N-tolyl | N-tolyl | H | a4 | H |
| 2-18-378 | N-tolyl | N-tolyl | t-bu | a4 | H |
| 2-18-379 | N-tolyl | N-tolyl | methyl | a4 | H |
| 2-18-380 | N-tolyl | N-tolyl | b1 | a4 | H |
| 2-18-381 | N-tolyl | N-tolyl | b2 | a4 | H |
| 2-18-382 | N-tolyl | N-tolyl | b3 | a4 | H |
| 2-18-383 | N-tolyl | N-tolyl | H | a5 | H |
| 2-18-384 | N-tolyl | N-tolyl | t-bu | a5 | H |
| 2-18-385 | N-tolyl | N-tolyl | methyl | a5 | H |
| 2-18-386 | N-tolyl | N-tolyl | b1 | a5 | H |
| 2-18-387 | N-tolyl | N-tolyl | b2 | a5 | H |
| 2-18-388 | N-tolyl | N-tolyl | b3 | a5 | H |
| 2-18-389 | N-tolyl | N-tolyl | H | a6 | H |
| 2-18-390 | N-tolyl | N-tolyl | t-bu | a6 | H |
| 2-18-391 | N-tolyl | N-tolyl | methyl | a6 | H |
| 2-18-392 | N-tolyl | N-tolyl | b1 | a6 | H |
| 2-18-393 | N-tolyl | N-tolyl | b2 | a6 | H |
| 2-18-394 | N-tolyl | N-tolyl | b3 | a6 | H |
| 2-18-395 | N-tolyl | N-tolyl | H | b1 | H |
| 2-18-396 | N-tolyl | N-tolyl | t-bu | b1 | H |
| 2-18-397 | N-tolyl | N-tolyl | methyl | b1 | H |
| 2-18-398 | N-tolyl | N-tolyl | b1 | b1 | H |
| 2-18-399 | N-tolyl | N-tolyl | b2 | b1 | H |
| 2-18-400 | N-tolyl | N-tolyl | b3 | b1 | H |
| 2-18-401 | N-tolyl | N-tolyl | H | b2 | H |
| 2-18-402 | N-tolyl | N-tolyl | t-bu | b2 | H |
| 2-18-403 | N-tolyl | N-tolyl | methyl | b2 | H |
| 2-18-404 | N-tolyl | N-tolyl | b1 | b2 | H |
| 2-18-405 | N-tolyl | N-tolyl | b2 | b2 | H |
| 2-18-406 | N-tolyl | N-tolyl | b3 | b2 | H |
| 2-18-407 | N-tolyl | N-tolyl | H | b3 | H |
| 2-18-408 | N-tolyl | N-tolyl | t-bu | b3 | H |
| 2-18-409 | N-tolyl | N-tolyl | methyl | b3 | H |
| 2-18-410 | N-tolyl | N-tolyl | b1 | b3 | H |
| 2-18-411 | N-tolyl | N-tolyl | b2 | b3 | H |
| 2-18-412 | N-tolyl | N-tolyl | b3 | b3 | H |
| 2-18-413 | N-tolyl | N-tolyl | H | b4 | H |
| 2-18-414 | N-tolyl | N-tolyl | t-bu | b4 | H |
| 2-18-415 | N-tolyl | N-tolyl | methyl | b4 | H |
| 2-18-416 | N-tolyl | N-tolyl | b1 | b4 | H |
| 2-18-417 | N-tolyl | N-tolyl | b2 | b4 | H |
| 2-18-418 | N-tolyl | N-tolyl | b3 | b4 | H |
| 2-18-419 | N-tolyl | N-tolyl | H | b5 | H |
| 2-18-420 | N-tolyl | N-tolyl | t-bu | b5 | H |
| 2-18-421 | N-tolyl | N-tolyl | methyl | b5 | H |
| 2-18-422 | N-tolyl | N-tolyl | b1 | b5 | H |
| 2-18-423 | N-tolyl | N-tolyl | b2 | b5 | H |
| 2-18-424 | N-tolyl | N-tolyl | b3 | b5 | H |
| 2-18-425 | N-tolyl | N-tolyl | H | b6 | H |
| 2-18-426 | N-tolyl | N-tolyl | t-bu | b6 | H |
| 2-18-427 | N-tolyl | N-tolyl | methyl | b6 | H |
| 2-18-428 | N-tolyl | N-tolyl | b1 | b6 | H |
| 2-18-429 | N-tolyl | N-tolyl | b2 | b6 | H |
| 2-18-430 | N-tolyl | N-tolyl | b3 | b6 | H |
| 2-18-431 | N-tolyl | N-tolyl | H | b7 | H |
| 2-18-432 | N-tolyl | N-tolyl | t-bu | b7 | H |
| 2-18-433 | N-tolyl | N-tolyl | methyl | b7 | H |
| 2-18-434 | N-tolyl | N-tolyl | b1 | b7 | H |
| 2-18-435 | N-tolyl | N-tolyl | b2 | b7 | H |
| 2-18-436 | N-tolyl | N-tolyl | b3 | b7 | H |
| 2-18-437 | N-tolyl | N-tolyl | H | b8 | H |
| 2-18-438 | N-tolyl | N-tolyl | t-bu | b8 | H |
| 2-18-439 | N-tolyl | N-tolyl | methyl | b8 | H |
| 2-18-440 | N-tolyl | N-tolyl | b1 | b8 | H |
| 2-18-441 | N-tolyl | N-tolyl | b2 | b8 | H |
| 2-18-442 | N-tolyl | N-tolyl | b3 | b8 | H |
| 2-18-443 | N-tolyl | N-tolyl | H | b9 | H |
| 2-18-444 | N-tolyl | N-tolyl | t-bu | b9 | H |
| 2-18-445 | N-tolyl | N-tolyl | methyl | b9 | H |
| 2-18-446 | N-tolyl | N-tolyl | b1 | b9 | H |
| 2-18-447 | N-tolyl | N-tolyl | b2 | b9 | H |
| 2-18-448 | N-tolyl | N-tolyl | b3 | b9 | H |
| 2-18-449 | N-tolyl | N-tolyl | H | b1 0 | H |
| 2-18-450 | N-tolyl | N-tolyl | t-bu | b1 0 | H |
| 2-18-451 | N-tolyl | N-tolyl | methyl | b1 0 | H |
| 2-18-452 | N-tolyl | N-tolyl | b1 | b1 0 | H |
| 2-18-453 | N-tolyl | N-tolyl | b2 | b1 0 | H |
| 2-18-454 | N-tolyl | N-tolyl | b3 | b1 0 | H |
| 2-18-455 | N-tolyl | N-tolyl | H | a1 | a1 |
| 2-18-456 | N-tolyl | N-tolyl | t-bu | a1 | a1 |
| 2-18-457 | N-tolyl | N-tolyl | methyl | a1 | a1 |
| 2-18-458 | N-tolyl | N-tolyl | b1 | a1 | a1 |
| 2-18-459 | N-tolyl | N-tolyl | b2 | a1 | a1 |
| 2-18-460 | N-tolyl | N-tolyl | b3 | a1 | a1 |
| 2-18-461 | N-tolyl | N-tolyl | H | a2 | a2 |
| 2-18-462 | N-tolyl | N-tolyl | t-bu | a2 | a2 |
| 2-18-463 | N-tolyl | N-tolyl | methyl | a2 | a2 |
| 2-18-464 | N-tolyl | N-tolyl | b1 | a2 | a2 |
| 2-18-465 | N-tolyl | N-tolyl | b2 | a2 | a2 |
| 2-18-466 | N-tolyl | N-tolyl | b3 | a2 | a2 |
| 2-18-467 | N-tolyl | N-tolyl | H | a3 | a3 |
| 2-18-468 | N-tolyl | N-tolyl | t-bu | a3 | a3 |
| 2-18-469 | N-tolyl | N-tolyl | methyl | a3 | a3 |
| 2-18-470 | N-tolyl | N-tolyl | b1 | a3 | a3 |
| 2-18-471 | N-tolyl | N-tolyl | b2 | a3 | a3 |
| 2-18-472 | N-tolyl | N-tolyl | b3 | a3 | a3 |
| 2-18-473 | N-tolyl | N-tolyl | H | b1 | b1 |
| 2-18-474 | N-tolyl | N-tolyl | t-bu | b1 | b1 |
| 2-18-475 | N-tolyl | N-tolyl | methyl | b1 | b1 |
| 2-18-476 | N-tolyl | N-tolyl | b1 | b1 | b1 |
| 2-18-477 | N-tolyl | N-tolyl | b2 | b1 | b1 |
| 2-18-478 | N-tolyl | N-tolyl | b3 | b1 | b1 |
| 2-18-479 | N-tolyl | N-tolyl | H | b2 | b2 |
| 2-18-480 | N-tolyl | N-tolyl | t-bu | b2 | b2 |
| 2-18-481 | N-tolyl | N-tolyl | methyl | b2 | b2 |
| 2-18-482 | N-tolyl | N-tolyl | b1 | b2 | b2 |
| 2-18-483 | N-tolyl | N-tolyl | b2 | b2 | b2 |
| 2-18-484 | N-tolyl | N-tolyl | b3 | b2 | b2 |
| 2-18-485 | N-tolyl | N-tolyl | H | b3 | b3 |
| 2-18-486 | N-tolyl | N-tolyl | t-bu | b3 | b3 |
| 2-18-487 | N-tolyl | N-tolyl | methyl | b3 | b3 |
| 2-18-488 | N-tolyl | N-tolyl | b1 | b3 | b3 |
| 2-18-489 | N-tolyl | N-tolyl | b2 | b3 | b3 |
| 2-18-490 | N-tolyl | N-tolyl | b3 | b3 | b3 |
| 2-18-491 | N-tolyl | N-tolyl | H | b5 | b5 |
| 2-18-492 | N-tolyl | N-tolyl | t-bu | b5 | b5 |
| 2-18-493 | N-tolyl | N-tolyl | methyl | b5 | b5 |
| 2-18-494 | N-tolyl | N-tolyl | b1 | b5 | b5 |
| 2-18-495 | N-tolyl | N-tolyl | b2 | b5 | b5 |
| 2-18-496 | N-tolyl | N-tolyl | b3 | b5 | b5 |
| 2-18-497 | N-tolyl | N-tolyl | H | b6 | b6 |
| 2-18-498 | N-tolyl | N-tolyl | t-bu | b6 | b6 |
| 2-18-499 | N-tolyl | N-tolyl | methyl | b6 | b6 |
| 2-18-500 | N-tolyl | N-tolyl | b1 | b6 | b6 |
| 2-18-501 | N-tolyl | N-tolyl | b2 | b6 | b6 |
| 2-18-502 | N-tolyl | N-tolyl | b3 | b6 | b6 |
| 2-18-503 | N-tolyl | N-tolyl | H | b7 | b7 |
| 2-18-504 | N-tolyl | N-tolyl | t-bu | b7 | b7 |
| 2-18-505 | N-tolyl | N-tolyl | methyl | b7 | b7 |
| 2-18-506 | N-tolyl | N-tolyl | b1 | b7 | b7 |
| 2-18-507 | N-tolyl | N-tolyl | b2 | b7 | b7 |
| 2-18-508 | N-tolyl | N-tolyl | b3 | b7 | b7 |
| 2-18-509 | N-Biph | N-Biph | H | H | H |
| 2-18-510 | N-Biph | N-Biph | t-bu | H | H |
| 2-18-511 | N-Biph | N-Biph | methyl | H | H |
| 2-18-512 | N-Biph | N-Biph | t-bu | t-bu | t-bu |
| 2-18-513 | N-Biph | N-Biph | methyl | methyl | methyl |
| 2-18-514 | N-Biph | N-Biph | b1 | H | H |
| 2-18-515 | N-Biph | N-Biph | b2 | H | H |
| 2-18-516 | N-Biph | N-Biph | b3 | H | H |
| 2-18-517 | N-Biph | N-Biph | H | H | a1 |
| 2-18-518 | N-Biph | N-Biph | t-bu | H | a1 |
| 2-18-519 | N-Biph | N-Biph | methyl | H | a1 |
| 2-18-520 | N-Biph | N-Biph | b1 | H | a1 |
| 2-18-521 | N-Biph | N-Biph | b2 | H | a1 |
| 2-18-522 | N-Biph | N-Biph | b3 | H | a1 |
| 2-18-523 | N-Biph | N-Biph | H | H | a2 |
| 2-18-524 | N-Biph | N-Biph | t-bu | H | a2 |
| 2-18-525 | N-Biph | N-Biph | methyl | H | a2 |
| 2-18-526 | N-Biph | N-Biph | b1 | H | a2 |
| 2-18-527 | N-Biph | N-Biph | b2 | H | a2 |
| 2-18-528 | N-Biph | N-Biph | b3 | H | a2 |
| 2-18-529 | N-Biph | N-Biph | H | H | a3 |
| 2-18-530 | N-Biph | N-Biph | t-bu | H | a3 |
| 2-18-531 | N-Biph | N-Biph | methyl | H | a3 |
| 2-18-532 | N-Biph | N-Biph | b1 | H | a3 |
| 2-18-533 | N-Biph | N-Biph | b2 | H | a3 |
| 2-18-534 | N-Biph | N-Biph | b3 | H | a3 |
| 2-18-535 | N-Biph | N-Biph | H | H | a4 |
| 2-18-536 | N-Biph | N-Biph | t-bu | H | a4 |
| 2-18-537 | N-Biph | N-Biph | methyl | H | a4 |
| 2-18-538 | N-Biph | N-Biph | b1 | H | a4 |
| 2-18-539 | N-Biph | N-Biph | b2 | H | a4 |
| 2-18-540 | N-Biph | N-Biph | b3 | H | a4 |
| 2-18-541 | N-Biph | N-Biph | H | H | a5 |
| 2-18-542 | N-Biph | N-Biph | t-bu | H | a5 |
| 2-18-543 | N-Biph | N-Biph | methyl | H | a5 |
| 2-18-544 | N-Biph | N-Biph | b1 | H | a5 |
| 2-18-545 | N-Biph | N-Biph | b2 | H | a5 |
| 2-18-546 | N-Biph | N-Biph | b3 | H | a5 |
| 2-18-547 | N-Biph | N-Biph | H | H | a6 |
| 2-18-548 | N-Biph | N-Biph | t-bu | H | a6 |
| 2-18-549 | N-Biph | N-Biph | methyl | H | a6 |
| 2-18-550 | N-Biph | N-Biph | b1 | H | a6 |
| 2-18-551 | N-Biph | N-Biph | b2 | H | a6 |
| 2-18-552 | N-Biph | N-Biph | b3 | H | a6 |
| 2-18-553 | N-Biph | N-Biph | H | H | b1 |
| 2-18-554 | N-Biph | N-Biph | t-bu | H | b1 |
| 2-18-555 | N-Biph | N-Biph | methyl | H | b1 |
| 2-18-556 | N-Biph | N-Biph | b1 | H | b1 |
| 2-18-557 | N-Biph | N-Biph | b2 | H | b1 |
| 2-18-558 | N-Biph | N-Biph | b3 | H | b1 |
| 2-18-559 | N-Biph | N-Biph | H | H | b2 |
| 2-18-560 | N-Biph | N-Biph | t-bu | H | b2 |
| 2-18-561 | N-Biph | N-Biph | methyl | H | b2 |
| 2-18-562 | N-Biph | N-Biph | b1 | H | b2 |
| 2-18-563 | N-Biph | N-Biph | b2 | H | b2 |
| 2-18-564 | N-Biph | N-Biph | b3 | H | b2 |
| 2-18-565 | N-Biph | N-Biph | H | H | b3 |
| 2-18-566 | N-Biph | N-Biph | t-bu | H | b3 |
| 2-18-567 | N-Biph | N-Biph | methyl | H | b3 |
| 2-18-568 | N-Biph | N-Biph | b1 | H | b3 |
| 2-18-569 | N-Biph | N-Biph | b2 | H | b3 |
| 2-18-570 | N-Biph | N-Biph | b3 | H | b3 |
| 2-18-571 | N-Biph | N-Biph | H | H | b4 |
| 2-18-572 | N-Biph | N-Biph | t-bu | H | b4 |
| 2-18-573 | N-Biph | N-Biph | methyl | H | b4 |
| 2-18-574 | N-Biph | N-Biph | b1 | H | b4 |
| 2-18-575 | N-Biph | N-Biph | b2 | H | b4 |
| 2-18-576 | N-Biph | N-Biph | b3 | H | b4 |
| 2-18-577 | N-Biph | N-Biph | H | H | b5 |
| 2-18-578 | N-Biph | N-Biph | t-bu | H | b5 |
| 2-18-579 | N-Biph | N-Biph | methyl | H | b5 |
| 2-18-580 | N-Biph | N-Biph | b1 | H | b5 |
| 2-18-581 | N-Biph | N-Biph | b2 | H | b5 |
| 2-18-582 | N-Biph | N-Biph | b3 | H | b5 |
| 2-18-583 | N-Biph | N-Biph | H | H | b6 |
| 2-18-584 | N-Biph | N-Biph | t-bu | H | b6 |
| 2-18-585 | N-Biph | N-Biph | methyl | H | b6 |
| 2-18-586 | N-Biph | N-Biph | b1 | H | b6 |
| 2-18-587 | N-Biph | N-Biph | b2 | H | b6 |
| 2-18-588 | N-Biph | N-Biph | b3 | H | b6 |
| 2-18-589 | N-Biph | N-Biph | H | H | b7 |
| 2-18-590 | N-Biph | N-Biph | t-bu | H | b7 |
| 2-18-591 | N-Biph | N-Biph | methyl | H | b7 |
| 2-18-592 | N-Biph | N-Biph | b1 | H | b7 |
| 2-18-593 | N-Biph | N-Biph | b2 | H | b7 |
| 2-18-594 | N-Biph | N-Biph | b3 | H | b7 |
| 2-18-595 | N-Biph | N-Biph | H | H | b8 |
| 2-18-596 | N-Biph | N-Biph | t-bu | H | b8 |
| 2-18-597 | N-Biph | N-Biph | methyl | H | b8 |
| 2-18-598 | N-Biph | N-Biph | b1 | H | b8 |
| 2-18-599 | N-Biph | N-Biph | b2 | H | b8 |
| 2-18-600 | N-Biph | N-Biph | b3 | H | b8 |
| 2-18-601 | N-Biph | N-Biph | H | H | b9 |
| 2-18-602 | N-Biph | N-Biph | t-bu | H | b9 |
| 2-18-603 | N-Biph | N-Biph | methyl | H | b9 |
| 2-18-604 | N-Biph | N-Biph | b1 | H | b9 |
| 2-18-605 | N-Biph | N-Biph | b2 | H | b9 |
| 2-18-606 | N-Biph | N-Biph | b3 | H | b9 |
| 2-18-607 | N-Biph | N-Biph | H | H | b1 0 |
| 2-18-608 | N-Biph | N-Biph | t-bu | H | b1 0 |
| 2-18-609 | N-Biph | N-Biph | methyl | H | b1 0 |
| 2-18-610 | N-Biph | N-Biph | b1 | H | b1 0 |
| 2-18-611 | N-Biph | N-Biph | b2 | H | b1 0 |
| 2-18-612 | N-Biph | N-Biph | b3 | H | b1 0 |
| 2-18-613 | N-Biph | N-Biph | H | a1 | H |
| 2-18-614 | N-Biph | N-Biph | t-bu | a1 | H |
| 2-18-615 | N-Biph | N-Biph | methyl | a1 | H |
| 2-18-616 | N-Biph | N-Biph | b1 | a1 | H |
| 2-18-617 | N-Biph | N-Biph | b2 | a1 | H |
| 2-18-618 | N-Biph | N-Biph | b3 | a1 | H |
| 2-18-619 | N-Biph | N-Biph | H | a2 | H |
| 2-18-620 | N-Biph | N-Biph | t-bu | a2 | H |
| 2-18-621 | N-Biph | N-Biph | methyl | a2 | H |
| 2-18-622 | N-Biph | N-Biph | b1 | a2 | H |
| 2-18-623 | N-Biph | N-Biph | b2 | a2 | H |
| 2-18-624 | N-Biph | N-Biph | b3 | a2 | H |
| 2-18-625 | N-Biph | N-Biph | H | a3 | H |
| 2-18-626 | N-Biph | N-Biph | t-bu | a3 | H |
| 2-18-627 | N-Biph | N-Biph | methyl | a3 | H |
| 2-18-628 | N-Biph | N-Biph | b1 | a3 | H |
| 2-18-629 | N-Biph | N-Biph | b2 | a3 | H |
| 2-18-630 | N-Biph | N-Biph | b3 | a3 | H |
| 2-18-631 | N-Biph | N-Biph | H | a4 | H |
| 2-18-632 | N-Biph | N-Biph | t-bu | a4 | H |
| 2-18-633 | N-Biph | N-Biph | methyl | a4 | H |
| 2-18-634 | N-Biph | N-Biph | b1 | a4 | H |
| 2-18-635 | N-Biph | N-Biph | b2 | a4 | H |
| 2-18-636 | N-Biph | N-Biph | b3 | a4 | H |
| 2-18-637 | N-Biph | N-Biph | H | a5 | H |
| 2-18-638 | N-Biph | N-Biph | t-bu | a5 | H |
| 2-18-639 | N-Biph | N-Biph | methyl | a5 | H |
| 2-18-640 | N-Biph | N-Biph | b1 | a5 | H |
| 2-18-641 | N-Biph | N-Biph | b2 | a5 | H |
| 2-18-642 | N-Biph | N-Biph | b3 | a5 | H |
| 2-18-643 | N-Biph | N-Biph | H | a6 | H |
| 2-18-644 | N-Biph | N-Biph | t-bu | a6 | H |
| 2-18-645 | N-Biph | N-Biph | methyl | a6 | H |
| 2-18-646 | N-Biph | N-Biph | b1 | a6 | H |
| 2-18-647 | N-Biph | N-Biph | b2 | a6 | H |
| 2-18-648 | N-Biph | N-Biph | b3 | a6 | H |
| 2-18-649 | N-Biph | N-Biph | H | b1 | H |
| 2-18-650 | N-Biph | N-Biph | t-bu | b1 | H |
| 2-18-651 | N-Biph | N-Biph | methyl | b1 | H |
| 2-18-652 | N-Biph | N-Biph | b1 | b1 | H |
| 2-18-653 | N-Biph | N-Biph | b2 | b1 | H |
| 2-18-654 | N-Biph | N-Biph | b3 | b1 | H |
| 2-18-655 | N-Biph | N-Biph | H | b2 | H |
| 2-18-656 | N-Biph | N-Biph | t-bu | b2 | H |
| 2-18-657 | N-Biph | N-Biph | methyl | b2 | H |
| 2-18-658 | N-Biph | N-Biph | b1 | b2 | H |
| 2-18-659 | N-Biph | N-Biph | b2 | b2 | H |
| 2-18-660 | N-Biph | N-Biph | b3 | b2 | H |
| 2-18-661 | N-Biph | N-Biph | H | b3 | H |
| 2-18-662 | N-Biph | N-Biph | t-bu | b3 | H |
| 2-18-663 | N-Biph | N-Biph | methyl | b3 | H |
| 2-18-664 | N-Biph | N-Biph | b1 | b3 | H |
| 2-18-665 | N-Biph | N-Biph | b2 | b3 | H |
| 2-18-666 | N-Biph | N-Biph | b3 | b3 | H |
| 2-18-667 | N-Biph | N-Biph | H | b4 | H |
| 2-18-668 | N-Biph | N-Biph | t-bu | b4 | H |
| 2-18-669 | N-Biph | N-Biph | methyl | b4 | H |
| 2-18-670 | N-Biph | N-Biph | b1 | b4 | H |
| 2-18-671 | N-Biph | N-Biph | b2 | b4 | H |
| 2-18-672 | N-Biph | N-Biph | b3 | b4 | H |
| 2-18-673 | N-Biph | N-Biph | H | b5 | H |
| 2-18-674 | N-Biph | N-Biph | t-bu | b5 | H |
| 2-18-675 | N-Biph | N-Biph | methyl | b5 | H |
| 2-18-676 | N-Biph | N-Biph | b1 | b5 | H |
| 2-18-677 | N-Biph | N-Biph | b2 | b5 | H |
| 2-18-678 | N-Biph | N-Biph | b3 | b5 | H |
| 2-18-679 | N-Biph | N-Biph | H | b6 | H |
| 2-18-680 | N-Biph | N-Biph | t-bu | b6 | H |
| 2-18-681 | N-Biph | N-Biph | methyl | b6 | H |
| 2-18-682 | N-Biph | N-Biph | b1 | b6 | H |
| 2-18-683 | N-Biph | N-Biph | b2 | b6 | H |
| 2-18-684 | N-Biph | N-Biph | b3 | b6 | H |
| 2-18-685 | N-Biph | N-Biph | H | b7 | H |
| 2-18-686 | N-Biph | N-Biph | t-bu | b7 | H |
| 2-18-687 | N-Biph | N-Biph | methyl | b7 | H |
| 2-18-688 | N-Biph | N-Biph | b1 | b7 | H |
| 2-18-689 | N-Biph | N-Biph | b2 | b7 | H |
| 2-18-690 | N-Biph | N-Biph | b3 | b7 | H |
| 2-18-691 | N-Biph | N-Biph | H | b8 | H |
| 2-18-692 | N-Biph | N-Biph | t-bu | b8 | H |
| 2-18-693 | N-Biph | N-Biph | methyl | b8 | H |
| 2-18-694 | N-Biph | N-Biph | b1 | b8 | H |
| 2-18-695 | N-Biph | N-Biph | b2 | b8 | H |
| 2-18-696 | N-Biph | N-Biph | b3 | b8 | H |
| 2-18-697 | N-Biph | N-Biph | H | b9 | H |
| 2-18-698 | N-Biph | N-Biph | t-bu | b9 | H |
| 2-18-699 | N-Biph | N-Biph | methyl | b9 | H |
| 2-18-700 | N-Biph | N-Biph | b1 | b9 | H |
| 2-18-701 | N-Biph | N-Biph | b2 | b9 | H |
| 2-18-702 | N-Biph | N-Biph | b3 | b9 | H |
| 2-18-703 | N-Biph | N-Biph | H | b1 0 | H |
| 2-18-704 | N-Biph | N-Biph | t-bu | b1 0 | H |
| 2-18-705 | N-Biph | N-Biph | methyl | b1 0 | H |
| 2-18-706 | N-Biph | N-Biph | b1 | b1 0 | H |
| 2-18-707 | N-Biph | N-Biph | b2 | b1 0 | H |
| 2-18-708 | N-Biph | N-Biph | b3 | b1 0 | H |
| 2-18-709 | N-Biph | N-Biph | H | a1 | a1 |
| 2-18-710 | N-Biph | N-Biph | t-bu | a1 | a1 |
| 2-18-711 | N-Biph | N-Biph | methyl | a1 | a1 |
| 2-18-712 | N-Biph | N-Biph | b1 | a1 | a1 |
| 2-18-713 | N-Biph | N-Biph | b2 | a1 | a1 |
| 2-18-714 | N-Biph | N-Biph | b3 | a1 | a1 |
| 2-18-715 | N-Biph | N-Biph | H | a2 | a2 |
| 2-18-716 | N-Biph | N-Biph | t-bu | a2 | a2 |
| 2-18-717 | N-Biph | N-Biph | methyl | a2 | a2 |
| 2-18-718 | N-Biph | N-Biph | b1 | a2 | a2 |
| 2-18-719 | N-Biph | N-Biph | b2 | a2 | a2 |
| 2-18-720 | N-Biph | N-Biph | b3 | a2 | a2 |
| 2-18-721 | N-Biph | N-Biph | H | a3 | a3 |
| 2-18-722 | N-Biph | N-Biph | t-bu | a3 | a3 |
| 2-18-723 | N-Biph | N-Biph | methyl | a3 | a3 |
| 2-18-724 | N-Biph | N-Biph | b1 | a3 | a3 |
| 2-18-725 | N-Biph | N-Biph | b2 | a3 | a3 |
| 2-18-726 | N-Biph | N-Biph | b3 | a3 | a3 |
| 2-18-727 | N-Biph | N-Biph | H | b1 | b1 |
| 2-18-728 | N-Biph | N-Biph | t-bu | b1 | b1 |
| 2-18-729 | N-Biph | N-Biph | methyl | b1 | b1 |
| 2-18-730 | N-Biph | N-Biph | b1 | b1 | b1 |
| 2-18-731 | N-Biph | N-Biph | b2 | b1 | b1 |
| 2-18-732 | N-Biph | N-Biph | b3 | b1 | b1 |
| 2-18-733 | N-Biph | N-Biph | H | b2 | b2 |
| 2-18-734 | N-Biph | N-Biph | t-bu | b2 | b2 |
| 2-18-735 | N-Biph | N-Biph | methyl | b2 | b2 |
| 2-18-736 | N-Biph | N-Biph | b1 | b2 | b2 |
| 2-18-737 | N-Biph | N-Biph | b2 | b2 | b2 |
| 2-18-738 | N-Biph | N-Biph | b3 | b2 | b2 |
| 2-18-739 | N-Biph | N-Biph | H | b3 | b3 |
| 2-18-740 | N-Biph | N-Biph | t-bu | b3 | b3 |
| 2-18-741 | N-Biph | N-Biph | methyl | b3 | b3 |
| 2-18-742 | N-Biph | N-Biph | b1 | b3 | b3 |
| 2-18-743 | N-Biph | N-Biph | b2 | b3 | b3 |
| 2-18-744 | N-Biph | N-Biph | b3 | b3 | b3 |
| 2-18-745 | N-Biph | N-Biph | H | b5 | b5 |
| 2-18-746 | N-Biph | N-Biph | t-bu | b5 | b5 |
| 2-18-747 | N-Biph | N-Biph | methyl | b5 | b5 |
| 2-18-748 | N-Biph | N-Biph | b1 | b5 | b5 |
| 2-18-749 | N-Biph | N-Biph | b2 | b5 | b5 |
| 2-18-750 | N-Biph | N-Biph | b3 | b5 | b5 |
| 2-18-751 | N-Biph | N-Biph | H | b6 | b6 |
| 2-18-752 | N-Biph | N-Biph | t-bu | b6 | b6 |
| 2-18-753 | N-Biph | N-Biph | methyl | b6 | b6 |
| 2-18-754 | N-Biph | N-Biph | b1 | b6 | b6 |
| 2-18-755 | N-Biph | N-Biph | b2 | b6 | b6 |
| 2-18-756 | N-Biph | N-Biph | b3 | b6 | b6 |
| 2-18-757 | N-Biph | N-Biph | H | b7 | b7 |
| 2-18-758 | N-Biph | N-Biph | t-bu | b7 | b7 |
| 2-18-759 | N-Biph | N-Biph | methyl | b7 | b7 |
| 2-18-760 | N-Biph | N-Biph | b1 | b7 | b7 |
| 2-18-761 | N-Biph | N-Biph | b2 | b7 | b7 |
| 2-18-762 | N-Biph | N-Biph | b3 | b7 | b7 |
| 2-18-763 | O | N-Ph | H | H | H |
| 2-18-764 | O | N-Ph | t-bu | H | H |
| 2-18-765 | O | N-Ph | methyl | H | H |
| 2-18-766 | O | N-Ph | t-bu | t-bu | t-bu |
| 2-18-767 | O | N-Ph | methyl | methyl | methyl |
| 2-18-768 | O | N-Ph | b1 | H | H |
| 2-18-769 | O | N-Ph | b2 | H | H |
| 2-18-770 | O | N-Ph | b3 | H | H |
| 2-18-771 | O | N-Ph | H | H | a1 |
| 2-18-772 | O | N-Ph | t-bu | H | a1 |
| 2-18-773 | O | N-Ph | methyl | H | a1 |
| 2-18-774 | O | N-Ph | b1 | H | a1 |
| 2-18-775 | O | N-Ph | b2 | H | a1 |
| 2-18-776 | O | N-Ph | b3 | H | a1 |
| 2-18-777 | O | N-Ph | H | H | a2 |
| 2-18-778 | O | N-Ph | t-bu | H | a2 |
| 2-18-779 | O | N-Ph | methyl | H | a2 |
| 2-18-780 | O | N-Ph | b1 | H | a2 |
| 2-18-781 | O | N-Ph | b2 | H | a2 |
| 2-18-782 | O | N-Ph | b3 | H | a2 |
| 2-18-783 | O | N-Ph | H | H | a3 |
| 2-18-784 | O | N-Ph | t-bu | H | a3 |
| 2-18-785 | O | N-Ph | methyl | H | a3 |
| 2-18-786 | O | N-Ph | b1 | H | a3 |
| 2-18-787 | O | N-Ph | b2 | H | a3 |
| 2-18-788 | O | N-Ph | b3 | H | a3 |
| 2-18-789 | O | N-Ph | H | H | a4 |
| 2-18-790 | O | N-Ph | t-bu | H | a4 |
| 2-18-791 | O | N-Ph | methyl | H | a4 |
| 2-18-792 | O | N-Ph | b1 | H | a4 |
| 2-18-793 | O | N-Ph | b2 | H | a4 |
| 2-18-794 | O | N-Ph | b3 | H | a4 |
| 2-18-795 | O | N-Ph | H | H | a5 |
| 2-18-796 | O | N-Ph | t-bu | H | a5 |
| 2-18-797 | O | N-Ph | methyl | H | a5 |
| 2-18-798 | O | N-Ph | b1 | H | a5 |
| 2-18-799 | O | N-Ph | b2 | H | a5 |
| 2-18-800 | O | N-Ph | b3 | H | a5 |
| 2-18-801 | O | N-Ph | H | H | a6 |
| 2-18-802 | O | N-Ph | t-bu | H | a6 |
| 2-18-803 | O | N-Ph | methyl | H | a6 |
| 2-18-804 | O | N-Ph | b1 | H | a6 |
| 2-18-805 | O | N-Ph | b2 | H | a6 |
| 2-18-806 | O | N-Ph | b3 | H | a6 |
| 2-18-807 | O | N-Ph | H | H | b1 |
| 2-18-808 | O | N-Ph | t-bu | H | b1 |
| 2-18-809 | O | N-Ph | methyl | H | b1 |
| 2-18-810 | O | N-Ph | b1 | H | b1 |
| 2-18-811 | O | N-Ph | b2 | H | b1 |
| 2-18-812 | O | N-Ph | b3 | H | b1 |
| 2-18-813 | O | N-Ph | H | H | b2 |
| 2-18-814 | O | N-Ph | t-bu | H | b2 |
| 2-18-815 | O | N-Ph | methyl | H | b2 |
| 2-18-816 | O | N-Ph | b1 | H | b2 |
| 2-18-817 | O | N-Ph | b2 | H | b2 |
| 2-18-818 | O | N-Ph | b3 | H | b2 |
| 2-18-819 | O | N-Ph | H | H | b3 |
| 2-18-820 | O | N-Ph | t-bu | H | b3 |
| 2-18-821 | O | N-Ph | methyl | H | b3 |
| 2-18-822 | O | N-Ph | b1 | H | b3 |
| 2-18-823 | O | N-Ph | b2 | H | b3 |
| 2-18-824 | O | N-Ph | b3 | H | b3 |
| 2-18-825 | O | N-Ph | H | H | b4 |
| 2-18-826 | O | N-Ph | t-bu | H | b4 |
| 2-18-827 | O | N-Ph | methyl | H | b4 |
| 2-18-828 | O | N-Ph | b1 | H | b4 |
| 2-18-829 | O | N-Ph | b2 | H | b4 |
| 2-18-830 | O | N-Ph | b3 | H | b4 |
| 2-18-831 | O | N-Ph | H | H | b5 |
| 2-18-832 | O | N-Ph | t-bu | H | b5 |
| 2-18-833 | O | N-Ph | methyl | H | b5 |
| 2-18-834 | O | N-Ph | b1 | H | b5 |
| 2-18-835 | O | N-Ph | b2 | H | b5 |
| 2-18-836 | O | N-Ph | b3 | H | b5 |
| 2-18-837 | O | N-Ph | H | H | b6 |
| 2-18-838 | O | N-Ph | t-bu | H | b6 |
| 2-18-839 | O | N-Ph | methyl | H | b6 |
| 2-18-840 | O | N-Ph | b1 | H | b6 |
| 2-18-841 | O | N-Ph | b2 | H | b6 |
| 2-18-842 | O | N-Ph | b3 | H | b6 |
| 2-18-843 | O | N-Ph | H | H | b7 |
| 2-18-844 | O | N-Ph | t-bu | H | b7 |
| 2-18-845 | O | N-Ph | methyl | H | b7 |
| 2-18-846 | O | N-Ph | b1 | H | b7 |
| 2-18-847 | O | N-Ph | b2 | H | b7 |
| 2-18-848 | O | N-Ph | b3 | H | b7 |
| 2-18-849 | O | N-Ph | H | H | b8 |
| 2-18-850 | O | N-Ph | t-bu | H | b8 |
| 2-18-851 | O | N-Ph | methyl | H | b8 |
| 2-18-852 | O | N-Ph | b1 | H | b8 |
| 2-18-853 | O | N-Ph | b2 | H | b8 |
| 2-18-854 | O | N-Ph | b3 | H | b8 |
| 2-18-855 | O | N-Ph | H | H | b9 |
| 2-18-856 | O | N-Ph | t-bu | H | b9 |
| 2-18-857 | O | N-Ph | methyl | H | b9 |
| 2-18-858 | O | N-Ph | b1 | H | b9 |
| 2-18-859 | O | N-Ph | b2 | H | b9 |
| 2-18-860 | O | N-Ph | b3 | H | b9 |
| 2-18-861 | O | N-Ph | H | H | b10 |
| 2-18-862 | O | N-Ph | t-bu | H | b10 |
| 2-18-863 | O | N-Ph | methyl | H | b10 |
| 2-18-864 | O | N-Ph | b1 | H | b10 |
| 2-18-865 | O | N-Ph | b2 | H | b10 |
| 2-18-866 | O | N-Ph | b3 | H | b10 |
| 2-18-867 | O | N-Ph | H | a1 | H |
| 2-18-868 | O | N-Ph | t-bu | a1 | H |
| 2-18-869 | O | N-Ph | methyl | a1 | H |
| 2-18-870 | O | N-Ph | b1 | a1 | H |
| 2-18-871 | O | N-Ph | b2 | a1 | H |
| 2-18-872 | O | N-Ph | b3 | a1 | H |
| 2-18-873 | O | N-Ph | H | a2 | H |
| 2-18-874 | O | N-Ph | t-bu | a2 | H |
| 2-18-875 | O | N-Ph | methyl | a2 | H |
| 2-18-876 | O | N-Ph | b1 | a2 | H |
| 2-18-877 | O | N-Ph | b2 | a2 | H |
| 2-18-878 | O | N-Ph | b3 | a2 | H |
| 2-18-879 | O | N-Ph | H | a3 | H |
| 2-18-880 | O | N-Ph | t-bu | a3 | H |
| 2-18-881 | O | N-Ph | methyl | a3 | H |
| 2-18-882 | O | N-Ph | b1 | a3 | H |
| 2-18-883 | O | N-Ph | b2 | a3 | H |
| 2-18-884 | O | N-Ph | b3 | a3 | H |
| 2-18-885 | O | N-Ph | H | a4 | H |
| 2-18-886 | O | N-Ph | t-bu | a4 | H |
| 2-18-887 | O | N-Ph | methyl | a4 | H |
| 2-18-888 | O | N-Ph | b1 | a4 | H |
| 2-18-889 | O | N-Ph | b2 | a4 | H |
| 2-18-890 | O | N-Ph | b3 | a4 | H |
| 2-18-891 | O | N-Ph | H | a5 | H |
| 2-18-892 | O | N-Ph | t-bu | a5 | H |
| 2-18-893 | O | N-Ph | methyl | a5 | H |
| 2-18-894 | O | N-Ph | b1 | a5 | H |
| 2-18-895 | O | N-Ph | b2 | a5 | H |
| 2-18-896 | O | N-Ph | b3 | a5 | H |
| 2-18-897 | O | N-Ph | H | a6 | H |
| 2-18-898 | O | N-Ph | t-bu | a6 | H |
| 2-18-899 | O | N-Ph | methyl | a6 | H |
| 2-18-900 | O | N-Ph | b1 | a6 | H |
| 2-18-901 | O | N-Ph | b2 | a6 | H |
| 2-18-902 | O | N-Ph | b3 | a6 | H |
| 2-18-903 | O | N-Ph | H | b1 | H |
| 2-18-904 | O | N-Ph | t-bu | b1 | H |
| 2-18-905 | O | N-Ph | methyl | b1 | H |
| 2-18-906 | O | N-Ph | b1 | b1 | H |
| 2-18-907 | O | N-Ph | b2 | b1 | H |
| 2-18-908 | O | N-Ph | b3 | b1 | H |
| 2-18-909 | O | N-Ph | H | b2 | H |
| 2-18-910 | O | N-Ph | t-bu | b2 | H |
| 2-18-911 | O | N-Ph | methyl | b2 | H |
| 2-18-912 | O | N-Ph | b1 | b2 | H |
| 2-18-913 | O | N-Ph | b2 | b2 | H |
| 2-18-914 | O | N-Ph | b3 | b2 | H |
| 2-18-915 | O | N-Ph | H | b3 | H |
| 2-18-916 | O | N-Ph | t-bu | b3 | H |
| 2-18-917 | O | N-Ph | methyl | b3 | H |
| 2-18-918 | O | N-Ph | b1 | b3 | H |
| 2-18-919 | O | N-Ph | b2 | b3 | H |
| 2-18-920 | O | N-Ph | b3 | b3 | H |
| 2-18-921 | O | N-Ph | H | b4 | H |
| 2-18-922 | O | N-Ph | t-bu | b4 | H |
| 2-18-923 | O | N-Ph | methyl | b4 | H |
| 2-18-924 | O | N-Ph | b1 | b4 | H |
| 2-18-925 | O | N-Ph | b2 | b4 | H |
| 2-18-926 | O | N-Ph | b3 | b4 | H |
| 2-18-927 | O | N-Ph | H | b5 | H |
| 2-18-928 | O | N-Ph | t-bu | b5 | H |
| 2-18-929 | O | N-Ph | methyl | b5 | H |
| 2-18-930 | O | N-Ph | b1 | b5 | H |
| 2-18-931 | O | N-Ph | b2 | b5 | H |
| 2-18-932 | O | N-Ph | b3 | b5 | H |
| 2-18-933 | O | N-Ph | H | b6 | H |
| 2-18-934 | O | N-Ph | t-bu | b6 | H |
| 2-18-935 | O | N-Ph | methyl | b6 | H |
| 2-18-936 | O | N-Ph | b1 | b6 | H |
| 2-18-937 | O | N-Ph | b2 | b6 | H |
| 2-18-938 | O | N-Ph | b3 | b6 | H |
| 2-18-939 | O | N-Ph | H | b7 | H |
| 2-18-940 | O | N-Ph | t-bu | b7 | H |
| 2-18-941 | O | N-Ph | methyl | b7 | H |
| 2-18-942 | O | N-Ph | b1 | b7 | H |
| 2-18-943 | O | N-Ph | b2 | b7 | H |
| 2-18-944 | O | N-Ph | b3 | b7 | H |
| 2-18-945 | O | N-Ph | H | b8 | H |
| 2-18-946 | O | N-Ph | t-bu | b8 | H |
| 2-18-947 | O | N-Ph | methyl | b8 | H |
| 2-18-948 | O | N-Ph | b1 | b8 | H |
| 2-18-949 | O | N-Ph | b2 | b8 | H |
| 2-18-950 | O | N-Ph | b3 | b8 | H |
| 2-18-951 | O | N-Ph | H | b9 | H |
| 2-18-952 | O | N-Ph | t-bu | b9 | H |
| 2-18-953 | O | N-Ph | methyl | b9 | H |
| 2-18-954 | O | N-Ph | b1 | b9 | H |
| 2-18-955 | O | N-Ph | b2 | b9 | H |
| 2-18-956 | O | N-Ph | b3 | b9 | H |
| 2-18-957 | O | N-Ph | H | b1 0 | H |
| 2-18-958 | O | N-Ph | t-bu | b1 0 | H |
| 2-18-959 | O | N-Ph | methyl | b1 0 | H |
| 2-18-960 | O | N-Ph | b1 | b1 0 | H |
| 2-18-961 | O | N-Ph | b2 | b1 0 | H |
| 2-18-962 | O | N-Ph | b3 | b1 0 | H |
| 2-18-963 | O | N-Ph | H | a1 | a1 |
| 2-18-964 | O | N-Ph | t-bu | a1 | a1 |
| 2-18-965 | O | N-Ph | methyl | a1 | a1 |
| 2-18-966 | O | N-Ph | b1 | a1 | a1 |
| 2-18-967 | O | N-Ph | b2 | a1 | a1 |
| 2-18-968 | O | N-Ph | b3 | a1 | a1 |
| 2-18-969 | O | N-Ph | H | a2 | a2 |
| 2-18-970 | O | N-Ph | t-bu | a2 | a2 |
| 2-18-971 | O | N-Ph | methyl | a2 | a2 |
| 2-18-972 | O | N-Ph | b1 | a2 | a2 |
| 2-18-973 | O | N-Ph | b2 | a2 | a2 |
| 2-18-974 | O | N-Ph | b3 | a2 | a2 |
| 2-18-975 | O | N-Ph | H | a3 | a3 |
| 2-18-976 | O | N-Ph | t-bu | a3 | a3 |
| 2-18-977 | O | N-Ph | methyl | a3 | a3 |
| 2-18-978 | O | N-Ph | b1 | a3 | a3 |
| 2-18-979 | O | N-Ph | b2 | a3 | a3 |
| 2-18-980 | O | N-Ph | b3 | a3 | a3 |
| 2-18-981 | O | N-Ph | H | b1 | b1 |
| 2-18-982 | O | N-Ph | t-bu | b1 | b1 |
| 2-18-983 | O | N-Ph | methyl | b1 | b1 |
| 2-18-984 | O | N-Ph | b1 | b1 | b1 |
| 2-18-985 | O | N-Ph | b2 | b1 | b1 |
| 2-18-986 | O | N-Ph | b3 | b1 | b1 |
| 2-18-987 | O | N-Ph | H | b2 | b2 |
| 2-18-988 | O | N-Ph | t-bu | b2 | b2 |
| 2-18-989 | O | N-Ph | methyl | b2 | b2 |
| 2-18-990 | O | N-Ph | b1 | b2 | b2 |
| 2-18-991 | O | N-Ph | b2 | b2 | b2 |
| 2-18-992 | O | N-Ph | b3 | b2 | b2 |
| 2-18-993 | O | N-Ph | H | b3 | b3 |
| 2-18-994 | O | N-Ph | t-bu | b3 | b3 |
| 2-18-995 | O | N-Ph | methyl | b3 | b3 |
| 2-18-996 | O | N-Ph | b1 | b3 | b3 |
| 2-18-997 | O | N-Ph | b2 | b3 | b3 |
| 2-18-998 | O | N-Ph | b3 | b3 | b3 |
| 2-18-999 | O | N-Ph | H | b5 | b5 |
| 2-18-1000 | O | N-Ph | t-bu | b5 | b5 |
| 2-18-1001 | O | N-Ph | methyl | b5 | b5 |
| 2-18-1002 | O | N-Ph | b1 | b5 | b5 |
| 2-18-1003 | O | N-Ph | b2 | b5 | b5 |
| 2-18-1004 | O | N-Ph | b3 | b5 | b5 |
| 2-18-1005 | O | N-Ph | H | b6 | b6 |
| 2-18-1006 | O | N-Ph | t-bu | b6 | b6 |
| 2-18-1007 | O | N-Ph | methyl | b6 | b6 |
| 2-18-1008 | O | N-Ph | b1 | b6 | b6 |
| 2-18-1009 | O | N-Ph | b2 | b6 | b6 |
| 2-18-1010 | O | N-Ph | b3 | b6 | b6 |
| 2-18-1011 | O | N-Ph | H | b7 | b7 |
| 2-18-1012 | O | N-Ph | t-bu | b7 | b7 |
| 2-18-1013 | O | N-Ph | methyl | b7 | b7 |
| 2-18-1014 | O | N-Ph | b1 | b7 | b7 |
| 2-18-1015 | O | N-Ph | b2 | b7 | b7 |
| 2-18-1016 | O | N-Ph | b3 | b7 | b7 |
| 2-18-1017 | S | N-Ph | H | H | H |
| 2-18-1018 | S | N-Ph | t-bu | H | H |
| 2-18-1019 | S | N-Ph | methyl | H | H |
| 2-18-1020 | S | N-Ph | t-bu | t-bu | t-bu |
| 2-18-1021 | S | N-Ph | methyl | methyl | methyl |
| 2-18-1022 | S | N-Ph | b1 | H | H |
| 2-18-1023 | S | N-Ph | b2 | H | H |
| 2-18-1024 | S | N-Ph | b3 | H | H |
| 2-18-1025 | S | N-Ph | H | H | a1 |
| 2-18-1026 | S | N-Ph | t-bu | H | a1 |
| 2-18-1027 | S | N-Ph | methyl | H | a1 |
| 2-18-1028 | S | N-Ph | b1 | H | a1 |
| 2-18-1029 | S | N-Ph | b2 | H | a1 |
| 2-18-1030 | S | N-Ph | b3 | H | a1 |
| 2-18-1031 | S | N-Ph | H | H | a2 |
| 2-18-1032 | S | N-Ph | t-bu | H | a2 |
| 2-18-1033 | S | N-Ph | methyl | H | a2 |
| 2-18-1034 | S | N-Ph | b1 | H | a2 |
| 2-18-1035 | S | N-Ph | b2 | H | a2 |
| 2-18-1036 | S | N-Ph | b3 | H | a2 |
| 2-18-1037 | S | N-Ph | H | H | a3 |
| 2-18-1038 | S | N-Ph | t-bu | H | a3 |
| 2-18-1039 | S | N-Ph | methyl | H | a3 |
| 2-18-1040 | S | N-Ph | b1 | H | a3 |
| 2-18-1041 | S | N-Ph | b2 | H | a3 |
| 2-18-1042 | S | N-Ph | b3 | H | a3 |
| 2-18-1043 | S | N-Ph | H | H | a4 |
| 2-18-1044 | S | N-Ph | t-bu | H | a4 |
| 2-18-1045 | S | N-Ph | methyl | H | a4 |
| 2-18-1046 | S | N-Ph | b1 | H | a4 |
| 2-18-1047 | S | N-Ph | b2 | H | a4 |
| 2-18-1048 | S | N-Ph | b3 | H | a4 |
| 2-18-1049 | S | N-Ph | H | H | a5 |
| 2-18-1050 | S | N-Ph | t-bu | H | a5 |
| 2-18-1051 | S | N-Ph | methyl | H | a5 |
| 2-18-1052 | S | N-Ph | b1 | H | a5 |
| 2-18-1053 | S | N-Ph | b2 | H | a5 |
| 2-18-1054 | S | N-Ph | b3 | H | a5 |
| 2-18-1055 | S | N-Ph | H | H | a6 |
| 2-18-1056 | S | N-Ph | t-bu | H | a6 |
| 2-18-1057 | S | N-Ph | methyl | H | a6 |
| 2-18-1058 | S | N-Ph | b1 | H | a6 |
| 2-18-1059 | S | N-Ph | b2 | H | a6 |
| 2-18-1060 | S | N-Ph | b3 | H | a6 |
| 2-18-1061 | S | N-Ph | H | H | b1 |
| 2-18-1062 | S | N-Ph | t-bu | H | b1 |
| 2-18-1063 | S | N-Ph | methyl | H | b1 |
| 2-18-1064 | S | N-Ph | b1 | H | b1 |
| 2-18-1065 | S | N-Ph | b2 | H | b1 |
| 2-18-1066 | S | N-Ph | b3 | H | b1 |
| 2-18-1067 | S | N-Ph | H | H | b2 |
| 2-18-1068 | S | N-Ph | t-bu | H | b2 |
| 2-18-1069 | S | N-Ph | methyl | H | b2 |
| 2-18-1070 | S | N-Ph | b1 | H | b2 |
| 2-18-1071 | S | N-Ph | b2 | H | b2 |
| 2-18-1072 | S | N-Ph | b3 | H | b2 |
| 2-18-1073 | S | N-Ph | H | H | b3 |
| 2-18-1074 | S | N-Ph | t-bu | H | b3 |
| 2-18-1075 | S | N-Ph | methyl | H | b3 |
| 2-18-1076 | S | N-Ph | b1 | H | b3 |
| 2-18-1077 | S | N-Ph | b2 | H | b3 |
| 2-18-1078 | S | N-Ph | b3 | H | b3 |
| 2-18-1079 | S | N-Ph | H | H | b4 |
| 2-18-1080 | S | N-Ph | t-bu | H | b4 |
| 2-18-1081 | S | N-Ph | methyl | H | b4 |
| 2-18-1082 | S | N-Ph | b1 | H | b4 |
| 2-18-1083 | S | N-Ph | b2 | H | b4 |
| 2-18-1084 | S | N-Ph | b3 | H | b4 |
| 2-18-1085 | S | N-Ph | H | H | b5 |
| 2-18-1086 | S | N-Ph | t-bu | H | b5 |
| 2-18-1087 | S | N-Ph | methyl | H | b5 |
| 2-18-1088 | S | N-Ph | b1 | H | b5 |
| 2-18-1089 | S | N-Ph | b2 | H | b5 |
| 2-18-1090 | S | N-Ph | b3 | H | b5 |
| 2-18-1091 | S | N-Ph | H | H | b6 |
| 2-18-1092 | S | N-Ph | t-bu | H | b6 |
| 2-18-1093 | S | N-Ph | methyl | H | b6 |
| 2-18-1094 | S | N-Ph | b1 | H | b6 |
| 2-18-1095 | S | N-Ph | b2 | H | b6 |
| 2-18-1096 | S | N-Ph | b3 | H | b6 |
| 2-18-1097 | S | N-Ph | H | H | b7 |
| 2-18-1098 | S | N-Ph | t-bu | H | b7 |
| 2-18-1099 | S | N-Ph | methyl | H | b7 |
| 2-18-1100 | S | N-Ph | b1 | H | b7 |
| 2-18-1101 | S | N-Ph | b2 | H | b7 |
| 2-18-1102 | S | N-Ph | b3 | H | b7 |
| 2-18-1103 | S | N-Ph | H | H | b8 |
| 2-18-1104 | S | N-Ph | t-bu | H | b8 |
| 2-18-1105 | S | N-Ph | methyl | H | b8 |
| 2-18-1106 | S | N-Ph | b1 | H | b8 |
| 2-18-1107 | S | N-Ph | b2 | H | b8 |
| 2-18-1108 | S | N-Ph | b3 | H | b8 |
| 2-18-1109 | S | N-Ph | H | H | b9 |
| 2-18-1110 | S | N-Ph | t-bu | H | b9 |
| 2-18-1111 | S | N-Ph | methyl | H | b9 |
| 2-18-1112 | S | N-Ph | b1 | H | b9 |
| 2-18-1113 | S | N-Ph | b2 | H | b9 |
| 2-18-1114 | S | N-Ph | b3 | H | b9 |
| 2-18-1115 | S | N-Ph | H | H | b10 |
| 2-18-1116 | S | N-Ph | t-bu | H | b10 |
| 2-18-1117 | S | N-Ph | methyl | H | b10 |
| 2-18-1118 | S | N-Ph | b1 | H | b10 |
| 2-18-1119 | S | N-Ph | b2 | H | b10 |
| 2-18-1120 | S | N-Ph | b3 | H | b10 |
| 2-18-1121 | S | N-Ph | H | a1 | H |
| 2-18-1122 | S | N-Ph | t-bu | a1 | H |
| 2-18-1123 | S | N-Ph | methyl | a1 | H |
| 2-18-1124 | S | N-Ph | b1 | a1 | H |
| 2-18-1125 | S | N-Ph | b2 | a1 | H |
| 2-18-1126 | S | N-Ph | b3 | a1 | H |
| 2-18-1127 | S | N-Ph | H | a2 | H |
| 2-18-1128 | S | N-Ph | t-bu | a2 | H |
| 2-18-1129 | S | N-Ph | methyl | a2 | H |
| 2-18-1130 | S | N-Ph | b1 | a2 | H |
| 2-18-1131 | S | N-Ph | b2 | a2 | H |
| 2-18-1132 | S | N-Ph | b3 | a2 | H |
| 2-18-1133 | S | N-Ph | H | a3 | H |
| 2-18-1134 | S | N-Ph | t-bu | a3 | H |
| 2-18-1135 | S | N-Ph | methyl | a3 | H |
| 2-18-1136 | S | N-Ph | b1 | a3 | H |
| 2-18-1137 | S | N-Ph | b2 | a3 | H |
| 2-18-1138 | S | N-Ph | b3 | a3 | H |
| 2-18-1139 | S | N-Ph | H | a4 | H |
| 2-18-1140 | S | N-Ph | t-bu | a4 | H |
| 2-18-1141 | S | N-Ph | methyl | a4 | H |
| 2-18-1142 | S | N-Ph | b1 | a4 | H |
| 2-18-1143 | S | N-Ph | b2 | a4 | H |
| 2-18-1144 | S | N-Ph | b3 | a4 | H |
| 2-18-1145 | S | N-Ph | H | a5 | H |
| 2-18-1146 | S | N-Ph | t-bu | a5 | H |
| 2-18-1147 | S | N-Ph | methyl | a5 | H |
| 2-18-1148 | S | N-Ph | b1 | a5 | H |
| 2-18-1149 | S | N-Ph | b2 | a5 | H |
| 2-18-1150 | S | N-Ph | b3 | a5 | H |
| 2-18-1151 | S | N-Ph | H | a6 | H |
| 2-18-1152 | S | N-Ph | t-bu | a6 | H |
| 2-18-1153 | S | N-Ph | methyl | a6 | H |
| 2-18-1154 | S | N-Ph | b1 | a6 | H |
| 2-18-1155 | S | N-Ph | b2 | a6 | H |
| 2-18-1156 | S | N-Ph | b3 | a6 | H |
| 2-18-1157 | S | N-Ph | H | b1 | H |
| 2-18-1158 | S | N-Ph | t-bu | b1 | H |
| 2-18-1159 | S | N-Ph | methyl | b1 | H |
| 2-18-1160 | S | N-Ph | b1 | b1 | H |
| 2-18-1161 | S | N-Ph | b2 | b1 | H |
| 2-18-1162 | S | N-Ph | b3 | b1 | H |
| 2-18-1163 | S | N-Ph | H | b2 | H |
| 2-18-1164 | S | N-Ph | t-bu | b2 | H |
| 2-18-1165 | S | N-Ph | methyl | b2 | H |
| 2-18-1166 | S | N-Ph | b1 | b2 | H |
| 2-18-1167 | S | N-Ph | b2 | b2 | H |
| 2-18-1168 | S | N-Ph | b3 | b2 | H |
| 2-18-1169 | S | N-Ph | H | b3 | H |
| 2-18-1170 | S | N-Ph | t-bu | b3 | H |
| 2-18-1171 | S | N-Ph | methyl | b3 | H |
| 2-18-1172 | S | N-Ph | b1 | b3 | H |
| 2-18-1173 | S | N-Ph | b2 | b3 | H |
| 2-18-1174 | S | N-Ph | b3 | b3 | H |
| 2-18-1175 | S | N-Ph | H | b4 | H |
| 2-18-1176 | S | N-Ph | t-bu | b4 | H |
| 2-18-1177 | S | N-Ph | methyl | b4 | H |
| 2-18-1178 | S | N-Ph | b1 | b4 | H |
| 2-18-1179 | S | N-Ph | b2 | b4 | H |
| 2-18-1180 | S | N-Ph | b3 | b4 | H |
| 2-18-1181 | S | N-Ph | H | b5 | H |
| 2-18-1182 | S | N-Ph | t-bu | b5 | H |
| 2-18-1183 | S | N-Ph | methyl | b5 | H |
| 2-18-1184 | S | N-Ph | b1 | b5 | H |
| 2-18-1185 | S | N-Ph | b2 | b5 | H |
| 2-18-1186 | S | N-Ph | b3 | b5 | H |
| 2-18-1187 | S | N-Ph | H | b6 | H |
| 2-18-1188 | S | N-Ph | t-bu | b6 | H |
| 2-18-1189 | S | N-Ph | methyl | b6 | H |
| 2-18-1190 | S | N-Ph | b1 | b6 | H |
| 2-18-1191 | S | N-Ph | b2 | b6 | H |
| 2-18-1192 | S | N-Ph | b3 | b6 | H |
| 2-18-1193 | S | N-Ph | H | b7 | H |
| 2-18-1194 | S | N-Ph | t-bu | b7 | H |
| 2-18-1195 | S | N-Ph | methyl | b7 | H |
| 2-18-1196 | S | N-Ph | b1 | b7 | H |
| 2-18-1197 | S | N-Ph | b2 | b7 | H |
| 2-18-1198 | S | N-Ph | b3 | b7 | H |
| 2-18-1199 | S | N-Ph | H | b8 | H |
| 2-18-1200 | S | N-Ph | t-bu | b8 | H |
| 2-18-1201 | S | N-Ph | methyl | b8 | H |
| 2-18-1202 | S | N-Ph | b1 | b8 | H |
| 2-18-1203 | S | N-Ph | b2 | b8 | H |
| 2-18-1204 | S | N-Ph | b3 | b8 | H |
| 2-18-1205 | S | N-Ph | H | b9 | H |
| 2-18-1206 | S | N-Ph | t-bu | b9 | H |
| 2-18-1207 | S | N-Ph | methyl | b9 | H |
| 2-18-1208 | S | N-Ph | b1 | b9 | H |
| 2-18-1209 | S | N-Ph | b2 | b9 | H |
| 2-18-1210 | S | N-Ph | b3 | b9 | H |
| 2-18-1211 | S | N-Ph | H | b10 | H |
| 2-18-1212 | S | N-Ph | t-bu | b10 | H |
| 2-18-1213 | S | N-Ph | methyl | b10 | H |
| 2-18-1214 | S | N-Ph | b1 | b10 | H |
| 2-18-1215 | S | N-Ph | b2 | b10 | H |
| 2-18-1216 | S | N-Ph | b3 | b10 | H |
| 2-18-1217 | S | N-Ph | H | a1 | a1 |
| 2-18-1218 | S | N-Ph | t-bu | a1 | a1 |
| 2-18-1219 | S | N-Ph | methyl | a1 | a1 |
| 2-18-1220 | S | N-Ph | b1 | a1 | a1 |
| 2-18-1221 | S | N-Ph | b2 | a1 | a1 |
| 2-18-1222 | S | N-Ph | b3 | a1 | a1 |
| 2-18-1223 | S | N-Ph | H | a2 | a2 |
| 2-18-1224 | S | N-Ph | t-bu | a2 | a2 |
| 2-18-1225 | S | N-Ph | methyl | a2 | a2 |
| 2-18-1226 | S | N-Ph | b1 | a2 | a2 |
| 2-18-1227 | S | N-Ph | b2 | a2 | a2 |
| 2-18-1228 | S | N-Ph | b3 | a2 | a2 |
| 2-18-1229 | S | N-Ph | H | a3 | a3 |
| 2-18-1230 | S | N-Ph | t-bu | a3 | a3 |
| 2-18-1231 | S | N-Ph | methyl | a3 | a3 |
| 2-18-1232 | S | N-Ph | b1 | a3 | a3 |
| 2-18-1233 | S | N-Ph | b2 | a3 | a3 |
| 2-18-1234 | S | N-Ph | b3 | a3 | a3 |
| 2-18-1235 | S | N-Ph | H | b1 | b1 |
| 2-18-1236 | S | N-Ph | t-bu | b1 | b1 |
| 2-18-1237 | S | N-Ph | methyl | b1 | b1 |
| 2-18-1238 | S | N-Ph | b1 | b1 | b1 |
| 2-18-1239 | S | N-Ph | b2 | b1 | b1 |
| 2-18-1240 | S | N-Ph | b3 | b1 | b1 |
| 2-18-1241 | S | N-Ph | H | b2 | b2 |
| 2-18-1242 | S | N-Ph | t-bu | b2 | b2 |
| 2-18-1243 | S | N-Ph | methyl | b2 | b2 |
| 2-18-1244 | S | N-Ph | b1 | b2 | b2 |
| 2-18-1245 | S | N-Ph | b2 | b2 | b2 |
| 2-18-1246 | S | N-Ph | b3 | b2 | b2 |
| 2-18-1247 | S | N-Ph | H | b3 | b3 |
| 2-18-1248 | S | N-Ph | t-bu | b3 | b3 |
| 2-18-1249 | S | N-Ph | methyl | b3 | b3 |
| 2-18-1250 | S | N-Ph | b1 | b3 | b3 |
| 2-18-1251 | S | N-Ph | b2 | b3 | b3 |
| 2-18-1252 | S | N-Ph | b3 | b3 | b3 |
| 2-18-1253 | S | N-Ph | H | b5 | b5 |
| 2-18-1254 | S | N-Ph | t-bu | b5 | b5 |
| 2-18-1255 | S | N-Ph | methyl | b5 | b5 |
| 2-18-1256 | S | N-Ph | b1 | b5 | b5 |
| 2-18-1257 | S | N-Ph | b2 | b5 | b5 |
| 2-18-1258 | S | N-Ph | b3 | b5 | b5 |
| 2-18-1259 | S | N-Ph | H | b6 | b6 |
| 2-18-1260 | S | N-Ph | t-bu | b6 | b6 |
| 2-18-1261 | S | N-Ph | methyl | b6 | b6 |
| 2-18-1262 | S | N-Ph | b1 | b6 | b6 |
| 2-18-1263 | S | N-Ph | b2 | b6 | b6 |
| 2-18-1264 | S | N-Ph | b3 | b6 | b6 |
| 2-18-1265 | S | N-Ph | H | b7 | b7 |
| 2-18-1266 | S | N-Ph | t-bu | b7 | b7 |
| 2-18-1267 | S | N-Ph | methyl | b7 | b7 |
| 2-18-1268 | S | N-Ph | b1 | b7 | b7 |
| 2-18-1269 | S | N-Ph | b2 | b7 | b7 |
| 2-18-1270 | S | N-Ph | b3 | b7 | b7 |

Group 2-19
The compounds are represented by the formula 2-19 below, wherein CY₁ and CY₃ are each a benzene group, X is B, and Y1, Y2, Y3, R2b, (R1)a1, (R3)a3 are each shown in the table 19 below, 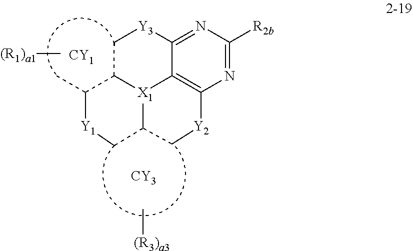
TABLE 19

| Compound | | | | | |
|---|---|---|---|---|---|
| No. | Y1 | Y2/Y3 | R2b | (R1)a1 | (R3)a3 |
| 2-19-001 | N—Ph | N—Ph | H | H | H |
| 2-19-002 | N—Ph | N—Ph | t-bu | H | H |
| 2-19-003 | N—Ph | N—Ph | methyl | H | H |
| 2-19-004 | N—Ph | N—Ph | t-bu | t-bu | t-bu |
| 2-19-005 | N—Ph | N—Ph | methyl | methyl | methyl |
| 2-19-006 | N—Ph | N—Ph | b1 | H | H |
| 2-19-007 | N—Ph | N—Ph | b2 | H | H |
| 2-19-008 | N—Ph | N—Ph | b3 | H | H |
| 2-19-009 | N—Ph | N—Ph | H | H | a1 |
| 2-19-010 | N—Ph | N—Ph | t-bu | H | a1 |
| 2-19-011 | N—Ph | N—Ph | methyl | H | a1 |
| 2-19-012 | N—Ph | N—Ph | b1 | H | a1 |
| 2-19-013 | N—Ph | N—Ph | b2 | H | a1 |
| 2-19-014 | N—Ph | N—Ph | b3 | H | a1 |
| 2-19-015 | N—Ph | N—Ph | H | H | a2 |
| 2-19-016 | N—Ph | N—Ph | t-bu | H | a2 |
| 2-19-017 | N—Ph | N—Ph | methyl | H | a2 |
| 2-19-018 | N—Ph | N—Ph | b1 | H | a2 |
| 2-19-019 | N—Ph | N—Ph | b2 | H | a2 |
| 2-19-020 | N—Ph | N—Ph | b3 | H | a2 |
| 2-19-021 | N—Ph | N—Ph | H | H | a3 |
| 2-19-022 | N—Ph | N—Ph | t-bu | H | a3 |
| 2-19-023 | N—Ph | N—Ph | methyl | H | a3 |
| 2-19-024 | N—Ph | N—Ph | b1 | H | a3 |
| 2-19-025 | N—Ph | N—Ph | b2 | H | a3 |
| 2-19-026 | N—Ph | N—Ph | b3 | H | a3 |
| 2-19-027 | N—Ph | N—Ph | H | H | a4 |
| 2-19-028 | N—Ph | N—Ph | t-bu | H | a4 |
| 2-19-029 | N—Ph | N—Ph | methyl | H | a4 |
| 2-19-030 | N—Ph | N—Ph | b1 | H | a4 |
| 2-19-031 | N—Ph | N—Ph | b2 | H | a4 |
| 2-19-032 | N—Ph | N—Ph | b3 | H | a4 |
| 2-19-033 | N—Ph | N—Ph | H | H | a5 |
| 2-19-034 | N—Ph | N—Ph | t-bu | H | a5 |
| 2-19-035 | N—Ph | N—Ph | methyl | H | a5 |
| 2-19-036 | N—Ph | N—Ph | b1 | H | a5 |
| 2-19-037 | N—Ph | N—Ph | b2 | H | a5 |
| 2-19-038 | N—Ph | N—Ph | b3 | H | a5 |
| 2-19-039 | N—Ph | N—Ph | H | H | a6 |
| 2-19-040 | N—Ph | N—Ph | t-bu | H | a6 |
| 2-19-041 | N—Ph | N—Ph | methyl | H | a6 |
| 2-19-042 | N—Ph | N—Ph | b1 | H | a6 |
| 2-19-043 | N—Ph | N—Ph | b2 | H | a6 |
| 2-19-044 | N—Ph | N—Ph | b3 | H | a6 |
| 2-19-045 | N—Ph | N—Ph | H | H | b1 |
| 2-19-046 | N—Ph | N—Ph | t-bu | H | b1 |
| 2-19-047 | N—Ph | N—Ph | methyl | H | b1 |
| 2-19-048 | N—Ph | N—Ph | b1 | H | b1 |
| 2-19-049 | N—Ph | N—Ph | b2 | H | b1 |
| 2-19-050 | N—Ph | N—Ph | b3 | H | b1 |
| 2-19-051 | N—Ph | N—Ph | H | H | b2 |
| 2-19-052 | N—Ph | N—Ph | t-bu | H | b2 |
| 2-19-053 | N—Ph | N—Ph | methyl | H | b2 |
| 2-19-054 | N—Ph | N—Ph | b1 | H | b2 |
| 2-19-055 | N—Ph | N—Ph | b2 | H | b2 |
| 2-19-056 | N—Ph | N—Ph | b3 | H | b2 |
| 2-19-057 | N—Ph | N—Ph | H | H | b3 |
| 2-19-058 | N—Ph | N—Ph | t-bu | H | b3 |
| 2-19-059 | N—Ph | N—Ph | methyl | H | b3 |
| 2-19-060 | N—Ph | N—Ph | b1 | H | b3 |
| 2-19-061 | N—Ph | N—Ph | b2 | H | b3 |
| 2-19-062 | N—Ph | N—Ph | b3 | H | b3 |
| 2-19-063 | N—Ph | N—Ph | H | H | b4 |
| 2-19-064 | N—Ph | N—Ph | t-bu | H | b4 |
| 2-19-065 | N—Ph | N—Ph | methyl | H | b4 |
| 2-19-066 | N—Ph | N—Ph | b1 | H | b4 |
| 2-19-067 | N—Ph | N—Ph | b2 | H | b4 |
| 2-19-068 | N—Ph | N—Ph | b3 | H | b4 |
| 2-19-069 | N—Ph | N—Ph | H | H | b5 |
| 2-19-070 | N—Ph | N—Ph | t-bu | H | b5 |
| 2-19-071 | N—Ph | N—Ph | methyl | H | b5 |
| 2-19-072 | N—Ph | N—Ph | b1 | H | b5 |
| 2-19-073 | N—Ph | N—Ph | b2 | H | b5 |
| 2-19-074 | N—Ph | N—Ph | b3 | H | b5 |
| 2-19-075 | N—Ph | N—Ph | H | H | b6 |
| 2-19-076 | N—Ph | N—Ph | t-bu | H | b6 |
| 2-19-077 | N—Ph | N—Ph | methyl | H | b6 |
| 2-19-078 | N—Ph | N—Ph | b1 | H | b6 |
| 2-19-079 | N—Ph | N—Ph | b2 | H | b6 |
| 2-19-080 | N—Ph | N—Ph | b3 | H | b6 |
| 2-19-081 | N—Ph | N—Ph | H | H | b7 |
| 2-19-082 | N—Ph | N—Ph | t-bu | H | b7 |
| 2-19-083 | N—Ph | N—Ph | methyl | H | b7 |
| 2-19-084 | N—Ph | N—Ph | b1 | H | b7 |
| 2-19-085 | N—Ph | N—Ph | b2 | H | b7 |
| 2-19-086 | N—Ph | N—Ph | b3 | H | b7 |
| 2-19-087 | N—Ph | N—Ph | H | H | b8 |
| 2-19-088 | N—Ph | N—Ph | t-bu | H | b8 |
| 2-19-089 | N—Ph | N—Ph | methyl | H | b8 |
| 2-19-090 | N—Ph | N—Ph | b1 | H | b8 |
| 2-19-091 | N—Ph | N—Ph | b2 | H | b8 |
| 2-19-092 | N—Ph | N—Ph | b3 | H | b8 |
| 2-19-093 | N—Ph | N—Ph | H | H | b9 |
| 2-19-094 | N—Ph | N—Ph | t-bu | H | b9 |
| 2-19-095 | N—Ph | N—Ph | methyl | H | b9 |
| 2-19-096 | N—Ph | N—Ph | b1 | H | b9 |
| 2-19-097 | N—Ph | N—Ph | b2 | H | b9 |
| 2-19-098 | N—Ph | N—Ph | b3 | H | b9 |
| 2-19-099 | N—Ph | N—Ph | H | H | b10 |
| 2-19-100 | N—Ph | N—Ph | t-bu | H | b10 |
| 2-19-101 | N—Ph | N—Ph | methyl | H | b10 |
| 2-19-102 | N—Ph | N—Ph | b1 | H | b10 |
| 2-19-103 | N—Ph | N—Ph | b2 | H | b10 |
| 2-19-104 | N—Ph | N—Ph | b3 | H | b10 |
| 2-19-105 | N—Ph | N—Ph | H | a1 | H |
| 2-19-106 | N—Ph | N—Ph | t-bu | a1 | H |
| 2-19-107 | N—Ph | N—Ph | methyl | a1 | H |
| 2-19-108 | N—Ph | N—Ph | b1 | a1 | H |
| 2-19-109 | N—Ph | N—Ph | b2 | a1 | H |
| 2-19-110 | N—Ph | N—Ph | b3 | a1 | H |
| 2-19-111 | N—Ph | N—Ph | H | a2 | H |
| 2-19-112 | N—Ph | N—Ph | t-bu | a2 | H |
| 2-19-113 | N—Ph | N—Ph | methyl | a2 | H |
| 2-19-114 | N—Ph | N—Ph | b1 | a2 | H |
| 2-19-115 | N—Ph | N—Ph | b2 | a2 | H |
| 2-19-116 | N—Ph | N—Ph | b3 | a2 | H |
| 2-19-117 | N—Ph | N—Ph | H | a3 | H |
| 2-19-118 | N—Ph | N—Ph | t-bu | a3 | H |
| 2-19-119 | N—Ph | N—Ph | methyl | a3 | H |
| 2-19-120 | N—Ph | N—Ph | b1 | a3 | H |
| 2-19-121 | N—Ph | N—Ph | b2 | a3 | H |
| 2-19-122 | N—Ph | N—Ph | b3 | a3 | H |
| 2-19-123 | N—Ph | N—Ph | H | a4 | H |
| 2-19-124 | N—Ph | N—Ph | t-bu | a4 | H |
| 2-19-125 | N—Ph | N—Ph | methyl | a4 | H |
| 2-19-126 | N—Ph | N—Ph | b1 | a4 | H |
| 2-19-127 | N—Ph | N—Ph | b2 | a4 | H |
| 2-19-128 | N—Ph | N—Ph | b3 | a4 | H |
| 2-19-129 | N—Ph | N—Ph | H | a5 | H |
| 2-19-130 | N—Ph | N—Ph | t-bu | a5 | H |
| 2-19-131 | N—Ph | N—Ph | methyl | a5 | H |
| 2-19-132 | N—Ph | N—Ph | b1 | a5 | H |
| 2-19-133 | N—Ph | N—Ph | b2 | a5 | H |
| 2-19-134 | N—Ph | N—Ph | b3 | a5 | H |
| 2-19-135 | N—Ph | N—Ph | H | a6 | H |
| 2-19-136 | N—Ph | N—Ph | t-bu | a6 | H |
| 2-19-137 | N—Ph | N—Ph | methyl | a6 | H |
| 2-19-138 | N—Ph | N—Ph | b1 | a6 | H |
| 2-19-139 | N—Ph | N—Ph | b2 | a6 | H |
| 2-19-140 | N—Ph | N—Ph | b3 | a6 | H |
| 2-19-141 | N—Ph | N—Ph | H | b1 | H |
| 2-19-142 | N—Ph | N—Ph | t-bu | b1 | H |
| 2-19-143 | N—Ph | N—Ph | methyl | b1 | H |
| 2-19-144 | N—Ph | N—Ph | b1 | b1 | H |
| 2-19-145 | N—Ph | N—Ph | b2 | b1 | H |
| 2-19-146 | N—Ph | N—Ph | b3 | b1 | H |
| 2-19-147 | N—Ph | N—Ph | H | b2 | H |
| 2-19-148 | N—Ph | N—Ph | t-bu | b2 | H |
| 2-19-149 | N—Ph | N—Ph | methyl | b2 | H |
| 2-19-150 | N—Ph | N—Ph | b1 | b2 | H |
| 2-19-151 | N—Ph | N—Ph | b2 | b2 | H |
| 2-19-152 | N—Ph | N—Ph | b3 | b2 | H |
| 2-19-153 | N—Ph | N—Ph | H | b3 | H |
| 2-19-154 | N—Ph | N—Ph | t-bu | b3 | H |
| 2-19-155 | N—Ph | N—Ph | methyl | b3 | H |
| 2-19-156 | N—Ph | N—Ph | b1 | b3 | H |
| 2-19-157 | N—Ph | N—Ph | b2 | b3 | H |
| 2-19-158 | N—Ph | N—Ph | b3 | b3 | H |
| 2-19-159 | N—Ph | N—Ph | H | b4 | H |
| 2-19-160 | N—Ph | N—Ph | t-bu | b4 | H |
| 2-19-161 | N—Ph | N—Ph | methyl | b4 | H |
| 2-19-162 | N—Ph | N—Ph | b1 | b4 | H |
| 2-19-163 | N—Ph | N—Ph | b2 | b4 | H |
| 2-19-164 | N—Ph | N—Ph | b3 | b4 | H |
| 2-19-165 | N—Ph | N—Ph | H | b5 | H |
| 2-19-166 | N—Ph | N—Ph | t-bu | b5 | H |
| 2-19-167 | N—Ph | N—Ph | methyl | b5 | H |
| 2-19-168 | N—Ph | N—Ph | b1 | b5 | H |
| 2-19-169 | N—Ph | N—Ph | b2 | b5 | H |
| 2-19-170 | N—Ph | N—Ph | b3 | b5 | H |
| 2-19-171 | N—Ph | N—Ph | H | b6 | H |
| 2-19-172 | N—Ph | N—Ph | t-bu | b6 | H |
| 2-19-173 | N—Ph | N—Ph | methyl | b6 | H |
| 2-19-174 | N—Ph | N—Ph | b1 | b6 | H |
| 2-19-175 | N—Ph | N—Ph | b2 | b6 | H |
| 2-19-176 | N—Ph | N—Ph | b3 | b6 | H |
| 2-19-177 | N—Ph | N—Ph | H | b7 | H |
| 2-19-178 | N—Ph | N—Ph | t-bu | b7 | H |
| 2-19-179 | N—Ph | N—Ph | methyl | b7 | H |
| 2-19-180 | N—Ph | N—Ph | b1 | b7 | H |
| 2-19-181 | N—Ph | N—Ph | b2 | b7 | H |
| 2-19-182 | N—Ph | N—Ph | b3 | b7 | H |
| 2-19-183 | N—Ph | N—Ph | H | b8 | H |
| 2-19-184 | N—Ph | N—Ph | t-bu | b8 | H |
| 2-19-185 | N—Ph | N—Ph | methyl | b8 | H |
| 2-19-186 | N—Ph | N—Ph | b1 | b8 | H |
| 2-19-187 | N—Ph | N—Ph | b2 | b8 | H |
| 2-19-188 | N—Ph | N—Ph | b3 | b8 | H |
| 2-19-189 | N—Ph | N—Ph | H | b9 | H |
| 2-19-190 | N—Ph | N—Ph | t-bu | b9 | H |
| 2-19-191 | N—Ph | N—Ph | methyl | b9 | H |
| 2-19-192 | N—Ph | N—Ph | b1 | b9 | H |
| 2-19-193 | N—Ph | N—Ph | b2 | b9 | H |
| 2-19-194 | N—Ph | N—Ph | b3 | b9 | H |
| 2-19-195 | N—Ph | N—Ph | H | b10 | H |
| 2-19-196 | N—Ph | N—Ph | t-bu | b10 | H |
| 2-19-197 | N—Ph | N—Ph | methyl | b10 | H |
| 2-19-198 | N—Ph | N—Ph | b1 | b10 | H |
| 2-19-199 | N—Ph | N—Ph | b2 | b10 | H |
| 2-19-200 | N—Ph | N—Ph | b3 | b10 | H |
| 2-19-201 | N—Ph | N—Ph | H | a1 | a1 |
| 2-19-202 | N—Ph | N—Ph | t-bu | a1 | a1 |
| 2-19-203 | N—Ph | N—Ph | methyl | a1 | a1 |
| 2-19-204 | N—Ph | N—Ph | b1 | a1 | a1 |
| 2-19-205 | N—Ph | N—Ph | b2 | a1 | a1 |
| 2-19-206 | N—Ph | N—Ph | b3 | a1 | a1 |
| 2-19-207 | N—Ph | N—Ph | H | a2 | a2 |
| 2-19-208 | N—Ph | N—Ph | t-bu | a2 | a2 |
| 2-19-209 | N—Ph | N—Ph | methyl | a2 | a2 |
| 2-19-210 | N—Ph | N—Ph | b1 | a2 | a2 |
| 2-19-211 | N—Ph | N—Ph | b2 | a2 | a2 |
| 2-19-212 | N—Ph | N—Ph | b3 | a2 | a2 |
| 2-19-213 | N—Ph | N—Ph | H | a3 | a3 |
| 2-19-214 | N—Ph | N—Ph | t-bu | a3 | a3 |
| 2-19-215 | N—Ph | N—Ph | methyl | a3 | a3 |
| 2-19-216 | N—Ph | N—Ph | b1 | a3 | a3 |
| 2-19-217 | N—Ph | N—Ph | b2 | a3 | a3 |
| 2-19-218 | N—Ph | N—Ph | b3 | a3 | a3 |
| 2-19-219 | N—Ph | N—Ph | H | b1 | b1 |
| 2-19-220 | N—Ph | N—Ph | t-bu | b1 | b1 |
| 2-19-221 | N—Ph | N—Ph | methyl | b1 | b1 |
| 2-19-222 | N—Ph | N—Ph | b1 | b1 | b1 |
| 2-19-223 | N—Ph | N—Ph | b2 | b1 | b1 |
| 2-19-224 | N—Ph | N—Ph | b3 | b1 | b1 |
| 2-19-225 | N—Ph | N—Ph | H | b2 | b2 |
| 2-19-226 | N—Ph | N—Ph | t-bu | b2 | b2 |
| 2-19-227 | N—Ph | N—Ph | methyl | b2 | b2 |
| 2-19-228 | N—Ph | N—Ph | b1 | b2 | b2 |
| 2-19-229 | N—Ph | N—Ph | b2 | b2 | b2 |
| 2-19-230 | N—Ph | N—Ph | b3 | b2 | b2 |
| 2-19-231 | N—Ph | N—Ph | H | b3 | b3 |
| 2-19-232 | N—Ph | N—Ph | t-bu | b3 | b3 |
| 2-19-233 | N—Ph | N—Ph | methyl | b3 | b3 |
| 2-19-234 | N—Ph | N—Ph | b1 | b3 | b3 |
| 2-19-235 | N—Ph | N—Ph | b2 | b3 | b3 |
| 2-19-236 | N—Ph | N—Ph | b3 | b3 | b3 |
| 2-19-237 | N—Ph | N—Ph | H | b5 | b5 |
| 2-19-238 | N—Ph | N—Ph | t-bu | b5 | b5 |
| 2-19-239 | N—Ph | N—Ph | methyl | b5 | b5 |
| 2-19-240 | N—Ph | N—Ph | b1 | b5 | b5 |
| 2-19-241 | N—Ph | N—Ph | b2 | b5 | b5 |
| 2-19-242 | N—Ph | N—Ph | b3 | b5 | b5 |
| 2-19-243 | N—Ph | N—Ph | H | b6 | b6 |
| 2-19-244 | N—Ph | N—Ph | t-bu | b6 | b6 |
| 2-19-245 | N—Ph | N—Ph | methyl | b6 | b6 |
| 2-19-246 | N—Ph | N—Ph | b1 | b6 | b6 |
| 2-19-247 | N—Ph | N—Ph | b2 | b6 | b6 |
| 2-19-248 | N—Ph | N—Ph | b3 | b6 | b6 |
| 2-19-249 | N—Ph | N—Ph | H | b7 | b7 |
| 2-19-250 | N—Ph | N—Ph | t-bu | b7 | b7 |
| 2-19-251 | N—Ph | N—Ph | methyl | b7 | b7 |
| 2-19-252 | N—Ph | N—Ph | b1 | b7 | b7 |
| 2-19-253 | N—Ph | N—Ph | b2 | b7 | b7 |
| 2-19-254 | N—Ph | N—Ph | b3 | b7 | b7 |
| 2-19-255 | N-tolyl | N-tolyl | H | H | H |
| 2-19-256 | N-tolyl | N-tolyl | t-bu | H | H |
| 2-19-257 | N-tolyl | N-tolyl | methyl | H | H |
| 2-19-258 | N-tolyl | N-tolyl | t-bu | t-bu | t-bu |
| 2-19-259 | N-tolyl | N-tolyl | methyl | methyl | methyl |
| 2-19-260 | N-tolyl | N-tolyl | b1 | H | H |
| 2-19-261 | N-tolyl | N-tolyl | b2 | H | H |
| 2-19-262 | N-tolyl | N-tolyl | b3 | H | H |
| 2-19-263 | N-tolyl | N-tolyl | H | H | a1 |
| 2-19-264 | N-tolyl | N-tolyl | t-bu | H | a1 |
| 2-19-265 | N-tolyl | N-tolyl | methyl | H | a1 |
| 2-19-266 | N-tolyl | N-tolyl | b1 | H | a1 |
| 2-19-267 | N-tolyl | N-tolyl | b2 | H | a1 |
| 2-19-268 | N-tolyl | N-tolyl | b3 | H | a1 |
| 2-19-269 | N-tolyl | N-tolyl | H | H | a2 |
| 2-19-270 | N-tolyl | N-tolyl | t-bu | H | a2 |
| 2-19-271 | N-tolyl | N-tolyl | methyl | H | a2 |
| 2-19-272 | N-tolyl | N-tolyl | b1 | H | a2 |
| 2-19-273 | N-tolyl | N-tolyl | b2 | H | a2 |
| 2-19-274 | N-tolyl | N-tolyl | b3 | H | a2 |
| 2-19-275 | N-tolyl | N-tolyl | H | H | a3 |
| 2-19-276 | N-tolyl | N-tolyl | t-bu | H | a3 |
| 2-19-277 | N-tolyl | N-tolyl | methyl | H | a3 |
| 2-19-278 | N-tolyl | N-tolyl | b1 | H | a3 |
| 2-19-279 | N-tolyl | N-tolyl | b2 | H | a3 |
| 2-19-280 | N-tolyl | N-tolyl | b3 | H | a3 |
| 2-19-281 | N-tolyl | N-tolyl | H | H | a4 |
| 2-19-282 | N-tolyl | N-tolyl | t-bu | H | a4 |
| 2-19-283 | N-tolyl | N-tolyl | methyl | H | a4 |
| 2-19-284 | N-tolyl | N-tolyl | b1 | H | a4 |
| 2-19-285 | N-tolyl | N-tolyl | b2 | H | a4 |
| 2-19-286 | N-tolyl | N-tolyl | b3 | H | a4 |
| 2-19-287 | N-tolyl | N-tolyl | H | H | a5 |
| 2-19-288 | N-tolyl | N-tolyl | t-bu | H | a5 |
| 2-19-289 | N-tolyl | N-tolyl | methyl | H | a5 |
| 2-19-290 | N-tolyl | N-tolyl | b1 | H | a5 |
| 2-19-291 | N-tolyl | N-tolyl | b2 | H | a5 |
| 2-19-292 | N-tolyl | N-tolyl | b3 | H | a5 |
| 2-19-293 | N-tolyl | N-tolyl | H | H | a6 |
| 2-19-294 | N-tolyl | N-tolyl | t-bu | H | a6 |
| 2-19-295 | N-tolyl | N-tolyl | methyl | H | a6 |
| 2-19-296 | N-tolyl | N-tolyl | b1 | H | a6 |
| 2-19-297 | N-tolyl | N-tolyl | b2 | H | a6 |
| 2-19-298 | N-tolyl | N-tolyl | b3 | H | a6 |
| 2-19-299 | N-tolyl | N-tolyl | H | H | b1 |
| 2-19-300 | N-tolyl | N-tolyl | t-bu | H | b1 |
| 2-19-301 | N-tolyl | N-tolyl | methyl | H | b1 |
| 2-19-302 | N-tolyl | N-tolyl | b1 | H | b1 |
| 2-19-303 | N-tolyl | N-tolyl | b2 | H | b1 |
| 2-19-304 | N-tolyl | N-tolyl | b3 | H | b1 |
| 2-19-305 | N-tolyl | N-tolyl | H | H | b2 |
| 2-19-306 | N-tolyl | N-tolyl | t-bu | H | b2 |
| 2-19-307 | N-tolyl | N-tolyl | methyl | H | b2 |
| 2-19-308 | N-tolyl | N-tolyl | b1 | H | b2 |
| 2-19-309 | N-tolyl | N-tolyl | b2 | H | b2 |
| 2-19-310 | N-tolyl | N-tolyl | b3 | H | b2 |
| 2-19-311 | N-tolyl | N-tolyl | H | H | b3 |
| 2-19-312 | N-tolyl | N-tolyl | t-bu | H | b3 |
| 2-19-313 | N-tolyl | N-tolyl | methyl | H | b3 |
| 2-19-314 | N-tolyl | N-tolyl | b1 | H | b3 |
| 2-19-315 | N-tolyl | N-tolyl | b2 | H | b3 |
| 2-19-316 | N-tolyl | N-tolyl | b3 | H | b3 |
| 2-19-317 | N-tolyl | N-tolyl | H | H | b4 |
| 2-19-318 | N-tolyl | N-tolyl | t-bu | H | b4 |
| 2-19-319 | N-tolyl | N-tolyl | methyl | H | b4 |
| 2-19-320 | N-tolyl | N-tolyl | b1 | H | b4 |
| 2-19-321 | N-tolyl | N-tolyl | b2 | H | b4 |
| 2-19-322 | N-tolyl | N-tolyl | b3 | H | b4 |
| 2-19-323 | N-tolyl | N-tolyl | H | H | b5 |
| 2-19-324 | N-tolyl | N-tolyl | t-bu | H | b5 |
| 2-19-325 | N-tolyl | N-tolyl | methyl | H | b5 |
| 2-19-326 | N-tolyl | N-tolyl | b1 | H | b5 |
| 2-19-327 | N-tolyl | N-tolyl | b2 | H | b5 |
| 2-19-328 | N-tolyl | N-tolyl | b3 | H | b5 |
| 2-19-329 | N-tolyl | N-tolyl | H | H | b6 |
| 2-19-330 | N-tolyl | N-tolyl | t-bu | H | b6 |
| 2-19-331 | N-tolyl | N-tolyl | methyl | H | b6 |
| 2-19-332 | N-tolyl | N-tolyl | b1 | H | b6 |
| 2-19-333 | N-tolyl | N-tolyl | b2 | H | b6 |
| 2-19-334 | N-tolyl | N-tolyl | b3 | H | b6 |
| 2-19-335 | N-tolyl | N-tolyl | H | H | b7 |
| 2-19-336 | N-tolyl | N-tolyl | t-bu | H | b7 |
| 2-19-337 | N-tolyl | N-tolyl | methyl | H | b7 |
| 2-19-338 | N-tolyl | N-tolyl | b1 | H | b7 |
| 2-19-339 | N-tolyl | N-tolyl | b2 | H | b7 |
| 2-19-340 | N-tolyl | N-tolyl | b3 | H | b7 |
| 2-19-341 | N-tolyl | N-tolyl | H | H | b8 |
| 2-19-342 | N-tolyl | N-tolyl | t-bu | H | b8 |
| 2-19-343 | N-tolyl | N-tolyl | methyl | H | b8 |
| 2-19-344 | N-tolyl | N-tolyl | b1 | H | b8 |
| 2-19-345 | N-tolyl | N-tolyl | b2 | H | b8 |
| 2-19-346 | N-tolyl | N-tolyl | b3 | H | b8 |
| 2-19-347 | N-tolyl | N-tolyl | H | H | b9 |
| 2-19-348 | N-tolyl | N-tolyl | t-bu | H | b9 |
| 2-19-349 | N-tolyl | N-tolyl | methyl | H | b9 |
| 2-19-350 | N-tolyl | N-tolyl | b1 | H | b9 |
| 2-19-351 | N-tolyl | N-tolyl | b2 | H | b9 |
| 2-19-352 | N-tolyl | N-tolyl | b3 | H | b9 |
| 2-19-353 | N-tolyl | N-tolyl | H | H | b10 |
| 2-19-354 | N-tolyl | N-tolyl | t-bu | H | b10 |
| 2-19-355 | N-tolyl | N-tolyl | methyl | H | b10 |
| 2-19-356 | N-tolyl | N-tolyl | b1 | H | b10 |
| 2-19-357 | N-tolyl | N-tolyl | b2 | H | b10 |
| 2-19-358 | N-tolyl | N-tolyl | b3 | H | b10 |
| 2-19-359 | N-tolyl | N-tolyl | H | a1 | H |
| 2-19-360 | N-tolyl | N-tolyl | t-bu | a1 | H |
| 2-19-361 | N-tolyl | N-tolyl | methyl | a1 | H |
| 2-19-362 | N-tolyl | N-tolyl | b1 | a1 | H |
| 2-19-363 | N-tolyl | N-tolyl | b2 | a1 | H |
| 2-19-364 | N-tolyl | N-tolyl | b3 | a1 | H |
| 2-19-365 | N-tolyl | N-tolyl | H | a2 | H |
| 2-19-366 | N-tolyl | N-tolyl | t-bu | a2 | H |
| 2-19-367 | N-tolyl | N-tolyl | methyl | a2 | H |
| 2-19-368 | N-tolyl | N-tolyl | b1 | a2 | H |
| 2-19-369 | N-tolyl | N-tolyl | b2 | a2 | H |
| 2-19-370 | N-tolyl | N-tolyl | b3 | a2 | H |
| 2-19-371 | N-tolyl | N-tolyl | H | a3 | H |
| 2-19-372 | N-tolyl | N-tolyl | t-bu | a3 | H |
| 2-19-373 | N-tolyl | N-tolyl | methyl | a3 | H |
| 2-19-374 | N-tolyl | N-tolyl | b1 | a3 | H |
| 2-19-375 | N-tolyl | N-tolyl | b2 | a3 | H |
| 2-19-376 | N-tolyl | N-tolyl | b3 | a3 | H |
| 2-19-377 | N-tolyl | N-tolyl | H | a4 | H |
| 2-19-378 | N-tolyl | N-tolyl | t-bu | a4 | H |
| 2-19-379 | N-tolyl | N-tolyl | methyl | a4 | H |
| 2-19-380 | N-tolyl | N-tolyl | b1 | a4 | H |
| 2-19-381 | N-tolyl | N-tolyl | b2 | a4 | H |
| 2-19-382 | N-tolyl | N-tolyl | b3 | a4 | H |
| 2-19-383 | N-tolyl | N-tolyl | H | a5 | H |
| 2-19-384 | N-tolyl | N-tolyl | t-bu | a5 | H |
| 2-19-385 | N-tolyl | N-tolyl | methyl | a5 | H |
| 2-19-386 | N-tolyl | N-tolyl | b1 | a5 | H |
| 2-19-387 | N-tolyl | N-tolyl | b2 | a5 | H |
| 2-19-388 | N-tolyl | N-tolyl | b3 | a5 | H |
| 2-19-389 | N-tolyl | N-tolyl | H | a6 | H |
| 2-19-390 | N-tolyl | N-tolyl | t-bu | a6 | H |
| 2-19-391 | N-tolyl | N-tolyl | methyl | a6 | H |
| 2-19-392 | N-tolyl | N-tolyl | b1 | a6 | H |
| 2-19-393 | N-tolyl | N-tolyl | b2 | a6 | H |
| 2-19-394 | N-tolyl | N-tolyl | b3 | a6 | H |
| 2-19-395 | N-tolyl | N-tolyl | H | b1 | H |
| 2-19-396 | N-tolyl | N-tolyl | t-bu | b1 | H |
| 2-19-397 | N-tolyl | N-tolyl | methyl | b1 | H |
| 2-19-398 | N-tolyl | N-tolyl | b1 | b1 | H |
| 2-19-399 | N-tolyl | N-tolyl | b2 | b1 | H |
| 2-19-400 | N-tolyl | N-tolyl | b3 | b1 | H |
| 2-19-401 | N-tolyl | N-tolyl | H | b2 | H |
| 2-19-402 | N-tolyl | N-tolyl | t-bu | b2 | H |
| 2-19-403 | N-tolyl | N-tolyl | methyl | b2 | H |
| 2-19-404 | N-tolyl | N-tolyl | b1 | b2 | H |
| 2-19-405 | N-tolyl | N-tolyl | b2 | b2 | H |
| 2-19-406 | N-tolyl | N-tolyl | b3 | b2 | H |
| 2-19-407 | N-tolyl | N-tolyl | H | b3 | H |
| 2-19-408 | N-tolyl | N-tolyl | t-bu | b3 | H |
| 2-19-409 | N-tolyl | N-tolyl | methyl | b3 | H |
| 2-19-410 | N-tolyl | N-tolyl | b1 | b3 | H |
| 2-19-411 | N-tolyl | N-tolyl | b2 | b3 | H |
| 2-19-412 | N-tolyl | N-tolyl | b3 | b3 | H |
| 2-19-413 | N-tolyl | N-tolyl | H | b4 | H |
| 2-19-414 | N-tolyl | N-tolyl | t-bu | b4 | H |
| 2-19-415 | N-tolyl | N-tolyl | methyl | b4 | H |
| 2-19-416 | N-tolyl | N-tolyl | b1 | b4 | H |
| 2-19-417 | N-tolyl | N-tolyl | b2 | b4 | H |
| 2-19-418 | N-tolyl | N-tolyl | b3 | b4 | H |
| 2-19-419 | N-tolyl | N-tolyl | H | b5 | H |
| 2-19-420 | N-tolyl | N-tolyl | t-bu | b5 | H |
| 2-19-421 | N-tolyl | N-tolyl | methyl | b5 | H |
| 2-19-422 | N-tolyl | N-tolyl | b1 | b5 | H |
| 2-19-423 | N-tolyl | N-tolyl | b2 | b5 | H |
| 2-19-424 | N-tolyl | N-tolyl | b3 | b5 | H |
| 2-19-425 | N-tolyl | N-tolyl | H | b6 | H |
| 2-19-426 | N-tolyl | N-tolyl | t-bu | b6 | H |
| 2-19-427 | N-tolyl | N-tolyl | methyl | b6 | H |
| 2-19-428 | N-tolyl | N-tolyl | b1 | b6 | H |
| 2-19-429 | N-tolyl | N-tolyl | b2 | b6 | H |
| 2-19-430 | N-tolyl | N-tolyl | b3 | b6 | H |
| 2-19-431 | N-tolyl | N-tolyl | H | b7 | H |
| 2-19-432 | N-tolyl | N-tolyl | t-bu | b7 | H |
| 2-19-433 | N-tolyl | N-tolyl | methyl | b7 | H |
| 2-19-434 | N-tolyl | N-tolyl | b1 | b7 | H |
| 2-19-435 | N-tolyl | N-tolyl | b2 | b7 | H |
| 2-19-436 | N-tolyl | N-tolyl | b3 | b7 | H |
| 2-19-437 | N-tolyl | N-tolyl | H | b8 | H |
| 2-19-438 | N-tolyl | N-tolyl | t-bu | b8 | H |
| 2-19-439 | N-tolyl | N-tolyl | methyl | b8 | H |
| 2-19-440 | N-tolyl | N-tolyl | b1 | b8 | H |
| 2-19-441 | N-tolyl | N-tolyl | b2 | b8 | H |
| 2-19-442 | N-tolyl | N-tolyl | b3 | b8 | H |
| 2-19-443 | N-tolyl | N-tolyl | H | b9 | H |
| 2-19-444 | N-tolyl | N-tolyl | t-bu | b9 | H |
| 2-19-445 | N-tolyl | N-tolyl | methyl | b9 | H |
| 2-19-446 | N-tolyl | N-tolyl | b1 | b9 | H |
| 2-19-447 | N-tolyl | N-tolyl | b2 | b9 | H |
| 2-19-448 | N-tolyl | N-tolyl | b3 | b9 | H |
| 2-19-449 | N-tolyl | N-tolyl | H | b10 | H |
| 2-19-450 | N-tolyl | N-tolyl | t-bu | b10 | H |
| 2-19-451 | N-tolyl | N-tolyl | methyl | b10 | H |
| 2-19-452 | N-tolyl | N-tolyl | b1 | b10 | H |
| 2-19-453 | N-tolyl | N-tolyl | b2 | b10 | H |
| 2-19-454 | N-tolyl | N-tolyl | b3 | b10 | H |
| 2-19-455 | N-tolyl | N-tolyl | H | a1 | a1 |
| 2-19-456 | N-tolyl | N-tolyl | t-bu | a1 | a1 |
| 2-19-457 | N-tolyl | N-tolyl | methyl | a1 | a1 |
| 2-19-458 | N-tolyl | N-tolyl | b1 | a1 | a1 |
| 2-19-459 | N-tolyl | N-tolyl | b2 | a1 | a1 |
| 2-19-460 | N-tolyl | N-tolyl | b3 | a1 | a1 |
| 2-19-461 | N-tolyl | N-tolyl | H | a2 | a2 |
| 2-19-462 | N-tolyl | N-tolyl | t-bu | a2 | a2 |
| 2-19-463 | N-tolyl | N-tolyl | methyl | a2 | a2 |
| 2-19-464 | N-tolyl | N-tolyl | b1 | a2 | a2 |
| 2-19-465 | N-tolyl | N-tolyl | b2 | a2 | a2 |
| 2-19-466 | N-tolyl | N-tolyl | b3 | a2 | a2 |
| 2-19-467 | N-tolyl | N-tolyl | H | a3 | a3 |
| 2-19-468 | N-tolyl | N-tolyl | t-bu | a3 | a3 |
| 2-19-469 | N-tolyl | N-tolyl | methyl | a3 | a3 |
| 2-19-470 | N-tolyl | N-tolyl | b1 | a3 | a3 |
| 2-19-471 | N-tolyl | N-tolyl | b2 | a3 | a3 |
| 2-19-472 | N-tolyl | N-tolyl | b3 | a3 | a3 |
| 2-19-473 | N-tolyl | N-tolyl | H | b1 | b1 |
| 2-19-474 | N-tolyl | N-tolyl | t-bu | b1 | b1 |
| 2-19-475 | N-tolyl | N-tolyl | methyl | b1 | b1 |
| 2-19-476 | N-tolyl | N-tolyl | b1 | b1 | b1 |
| 2-19-477 | N-tolyl | N-tolyl | b2 | b1 | b1 |
| 2-19-478 | N-tolyl | N-tolyl | b3 | b1 | b1 |
| 2-19-479 | N-tolyl | N-tolyl | H | b2 | b2 |
| 2-19-480 | N-tolyl | N-tolyl | t-bu | b2 | b2 |
| 2-19-481 | N-tolyl | N-tolyl | methyl | b2 | b2 |
| 2-19-482 | N-tolyl | N-tolyl | b1 | b2 | b2 |
| 2-19-483 | N-tolyl | N-tolyl | b2 | b2 | b2 |
| 2-19-484 | N-tolyl | N-tolyl | b3 | b2 | b2 |
| 2-19-485 | N-tolyl | N-tolyl | H | b3 | b3 |
| 2-19-486 | N-tolyl | N-tolyl | t-bu | b3 | b3 |
| 2-19-487 | N-tolyl | N-tolyl | methyl | b3 | b3 |
| 2-19-488 | N-tolyl | N-tolyl | b1 | b3 | b3 |
| 2-19-489 | N-tolyl | N-tolyl | b2 | b3 | b3 |
| 2-19-490 | N-tolyl | N-tolyl | b3 | b3 | b3 |
| 2-19-491 | N-tolyl | N-tolyl | H | b5 | b5 |
| 2-19-492 | N-tolyl | N-tolyl | t-bu | b5 | b5 |
| 2-19-493 | N-tolyl | N-tolyl | methyl | b5 | b5 |
| 2-19-494 | N-tolyl | N-tolyl | b1 | b5 | b5 |
| 2-19-495 | N-tolyl | N-tolyl | b2 | b5 | b5 |
| 2-19-496 | N-tolyl | N-tolyl | b3 | b5 | b5 |
| 2-19-497 | N-tolyl | N-tolyl | H | b6 | b6 |
| 2-19-498 | N-tolyl | N-tolyl | t-bu | b6 | b6 |
| 2-19-499 | N-tolyl | N-tolyl | methyl | b6 | b6 |
| 2-19-500 | N-tolyl | N-tolyl | b1 | b6 | b6 |
| 2-19-501 | N-tolyl | N-tolyl | b2 | b6 | b6 |
| 2-19-502 | N-tolyl | N-tolyl | b3 | b6 | b6 |
| 2-19-503 | N-tolyl | N-tolyl | H | b7 | b7 |
| 2-19-504 | N-tolyl | N-tolyl | t-bu | b7 | b7 |
| 2-19-505 | N-tolyl | N-tolyl | methyl | b7 | b7 |
| 2-19-506 | N-tolyl | N-tolyl | b1 | b7 | b7 |
| 2-19-507 | N-tolyl | N-tolyl | b2 | b7 | b7 |
| 2-19-508 | N-tolyl | N-tolyl | b3 | b7 | b7 |
| 2-19-509 | N-Biph | N-Biph | H | H | H |
| 2-19-510 | N-Biph | N-Biph | t-bu | H | H |
| 2-19-511 | N-Biph | N-Biph | methyl | H | H |
| 2-19-512 | N-Biph | N-Biph | t-bu | t-bu | t-bu |
| 2-19-513 | N-Biph | N-Biph | methyl | methyl | methyl |
| 2-19-514 | N-Biph | N-Biph | b1 | H | H |
| 2-19-515 | N-Biph | N-Biph | b2 | H | H |
| 2-19-516 | N-Biph | N-Biph | b3 | H | H |
| 2-19-517 | N-Biph | N-Biph | H | H | a1 |
| 2-19-518 | N-Biph | N-Biph | t-bu | H | a1 |
| 2-19-519 | N-Biph | N-Biph | methyl | H | a1 |
| 2-19-520 | N-Biph | N-Biph | b1 | H | a1 |
| 2-19-521 | N-Biph | N-Biph | b2 | H | a1 |
| 2-19-522 | N-Biph | N-Biph | b3 | H | a1 |
| 2-19-523 | N-Biph | N-Biph | H | H | a2 |
| 2-19-524 | N-Biph | N-Biph | t-bu | H | a2 |
| 2-19-525 | N-Biph | N-Biph | methyl | H | a2 |
| 2-19-526 | N-Biph | N-Biph | b1 | H | a2 |
| 2-19-527 | N-Biph | N-Biph | b2 | H | a2 |
| 2-19-528 | N-Biph | N-Biph | b3 | H | a2 |
| 2-19-529 | N-Biph | N-Biph | H | H | a3 |
| 2-19-530 | N-Biph | N-Biph | t-bu | H | a3 |
| 2-19-531 | N-Biph | N-Biph | methyl | H | a3 |
| 2-19-532 | N-Biph | N-Biph | b1 | H | a3 |
| 2-19-533 | N-Biph | N-Biph | b2 | H | a3 |
| 2-19-534 | N-Biph | N-Biph | b3 | H | a3 |
| 2-19-535 | N-Biph | N-Biph | H | H | a4 |
| 2-19-536 | N-Biph | N-Biph | t-bu | H | a4 |
| 2-19-537 | N-Biph | N-Biph | methyl | H | a4 |
| 2-19-538 | N-Biph | N-Biph | b1 | H | a4 |
| 2-19-539 | N-Biph | N-Biph | b2 | H | a4 |
| 2-19-540 | N-Biph | N-Biph | b3 | H | a4 |
| 2-19-541 | N-Biph | N-Biph | H | H | a5 |
| 2-19-542 | N-Biph | N-Biph | t-bu | H | a5 |
| 2-19-543 | N-Biph | N-Biph | methyl | H | a5 |
| 2-19-544 | N-Biph | N-Biph | b1 | H | a5 |
| 2-19-545 | N-Biph | N-Biph | b2 | H | a5 |
| 2-19-546 | N-Biph | N-Biph | b3 | H | a5 |
| 2-19-547 | N-Biph | N-Biph | H | H | a6 |
| 2-19-548 | N-Biph | N-Biph | t-bu | H | a6 |
| 2-19-549 | N-Biph | N-Biph | methyl | H | a6 |
| 2-19-550 | N-Biph | N-Biph | b1 | H | a6 |
| 2-19-551 | N-Biph | N-Biph | b2 | H | a6 |
| 2-19-552 | N-Biph | N-Biph | b3 | H | a6 |
| 2-19-553 | N-Biph | N-Biph | H | H | b1 |
| 2-19-554 | N-Biph | N-Biph | t-bu | H | b1 |
| 2-19-555 | N-Biph | N-Biph | methyl | H | b1 |
| 2-19-556 | N-Biph | N-Biph | b1 | H | b1 |
| 2-19-557 | N-Biph | N-Biph | b2 | H | b1 |
| 2-19-558 | N-Biph | N-Biph | b3 | H | b1 |
| 2-19-559 | N-Biph | N-Biph | H | H | b2 |
| 2-19-560 | N-Biph | N-Biph | t-bu | H | b2 |
| 2-19-561 | N-Biph | N-Biph | methyl | H | b2 |
| 2-19-562 | N-Biph | N-Biph | b1 | H | b2 |
| 2-19-563 | N-Biph | N-Biph | b2 | H | b2 |
| 2-19-564 | N-Biph | N-Biph | b3 | H | b2 |
| 2-19-565 | N-Biph | N-Biph | H | H | b3 |
| 2-19-566 | N-Biph | N-Biph | t-bu | H | b3 |
| 2-19-567 | N-Biph | N-Biph | methyl | H | b3 |
| 2-19-568 | N-Biph | N-Biph | b1 | H | b3 |
| 2-19-569 | N-Biph | N-Biph | b2 | H | b3 |
| 2-19-570 | N-Biph | N-Biph | b3 | H | b3 |
| 2-19-571 | N-Biph | N-Biph | H | H | b4 |
| 2-19-572 | N-Biph | N-Biph | t-bu | H | b4 |
| 2-19-573 | N-Biph | N-Biph | methyl | H | b4 |
| 2-19-574 | N-Biph | N-Biph | b1 | H | b4 |
| 2-19-575 | N-Biph | N-Biph | b2 | H | b4 |
| 2-19-576 | N-Biph | N-Biph | b3 | H | b4 |
| 2-19-577 | N-Biph | N-Biph | H | H | b5 |
| 2-19-578 | N-Biph | N-Biph | t-bu | H | b5 |
| 2-19-579 | N-Biph | N-Biph | methyl | H | b5 |
| 2-19-580 | N-Biph | N-Biph | b1 | H | b5 |
| 2-19-581 | N-Biph | N-Biph | b2 | H | b5 |
| 2-19-582 | N-Biph | N-Biph | b3 | H | b5 |
| 2-19-583 | N-Biph | N-Biph | H | H | b6 |
| 2-19-584 | N-Biph | N-Biph | t-bu | H | b6 |
| 2-19-585 | N-Biph | N-Biph | methyl | H | b6 |
| 2-19-586 | N-Biph | N-Biph | b1 | H | b6 |
| 2-19-587 | N-Biph | N-Biph | b2 | H | b6 |
| 2-19-588 | N-Biph | N-Biph | b3 | H | b6 |
| 2-19-589 | N-Biph | N-Biph | H | H | b7 |
| 2-19-590 | N-Biph | N-Biph | t-bu | H | b7 |
| 2-19-591 | N-Biph | N-Biph | methyl | H | b7 |
| 2-19-592 | N-Biph | N-Biph | b1 | H | b7 |
| 2-19-593 | N-Biph | N-Biph | b2 | H | b7 |
| 2-19-594 | N-Biph | N-Biph | b3 | H | b7 |
| 2-19-595 | N-Biph | N-Biph | H | H | b8 |
| 2-19-596 | N-Biph | N-Biph | t-bu | H | b8 |
| 2-19-597 | N-Biph | N-Biph | methyl | H | b8 |
| 2-19-598 | N-Biph | N-Biph | b1 | H | b8 |
| 2-19-599 | N-Biph | N-Biph | b2 | H | b8 |
| 2-19-600 | N-Biph | N-Biph | b3 | H | b8 |
| 2-19-601 | N-Biph | N-Biph | H | H | b9 |
| 2-19-602 | N-Biph | N-Biph | t-bu | H | b9 |
| 2-19-603 | N-Biph | N-Biph | methyl | H | b9 |
| 2-19-604 | N-Biph | N-Biph | b1 | H | b9 |
| 2-19-605 | N-Biph | N-Biph | b2 | H | b9 |
| 2-19-606 | N-Biph | N-Biph | b3 | H | b9 |
| 2-19-607 | N-Biph | N-Biph | H | H | b10 |
| 2-19-608 | N-Biph | N-Biph | t-bu | H | b10 |
| 2-19-609 | N-Biph | N-Biph | methyl | H | b10 |
| 2-19-610 | N-Biph | N-Biph | b1 | H | b10 |
| 2-19-611 | N-Biph | N-Biph | b2 | H | b10 |
| 2-19-612 | N-Biph | N-Biph | b3 | H | b10 |
| 2-19-613 | N-Biph | N-Biph | H | a1 | H |
| 2-19-614 | N-Biph | N-Biph | t-bu | a1 | H |
| 2-19-615 | N-Biph | N-Biph | methyl | a1 | H |
| 2-19-616 | N-Biph | N-Biph | b1 | a1 | H |
| 2-19-617 | N-Biph | N-Biph | b2 | a1 | H |
| 2-19-618 | N-Biph | N-Biph | b3 | a1 | H |
| 2-19-619 | N-Biph | N-Biph | H | a2 | H |
| 2-19-620 | N-Biph | N-Biph | t-bu | a2 | H |
| 2-19-621 | N-Biph | N-Biph | methyl | a2 | H |
| 2-19-622 | N-Biph | N-Biph | b1 | a2 | H |
| 2-19-623 | N-Biph | N-Biph | b2 | a2 | H |
| 2-19-624 | N-Biph | N-Biph | b3 | a2 | H |
| 2-19-625 | N-Biph | N-Biph | H | a3 | H |
| 2-19-626 | N-Biph | N-Biph | t-bu | a3 | H |
| 2-19-627 | N-Biph | N-Biph | methyl | a3 | H |
| 2-19-628 | N-Biph | N-Biph | b1 | a3 | H |
| 2-19-629 | N-Biph | N-Biph | b2 | a3 | H |
| 2-19-630 | N-Biph | N-Biph | b3 | a3 | H |
| 2-19-631 | N-Biph | N-Biph | H | a4 | H |
| 2-19-632 | N-Biph | N-Biph | t-bu | a4 | H |
| 2-19-633 | N-Biph | N-Biph | methyl | a4 | H |
| 2-19-634 | N-Biph | N-Biph | b1 | a4 | H |
| 2-19-635 | N-Biph | N-Biph | b2 | a4 | H |
| 2-19-636 | N-Biph | N-Biph | b3 | a4 | H |
| 2-19-637 | N-Biph | N-Biph | H | a5 | H |
| 2-19-638 | N-Biph | N-Biph | t-bu | a5 | H |
| 2-19-639 | N-Biph | N-Biph | methyl | a5 | H |
| 2-19-640 | N-Biph | N-Biph | b1 | a5 | H |
| 2-19-641 | N-Biph | N-Biph | b2 | a5 | H |
| 2-19-642 | N-Biph | N-Biph | b3 | a5 | H |
| 2-19-643 | N-Biph | N-Biph | H | a6 | H |
| 2-19-644 | N-Biph | N-Biph | t-bu | a6 | H |
| 2-19-645 | N-Biph | N-Biph | methyl | a6 | H |
| 2-19-646 | N-Biph | N-Biph | b1 | a6 | H |
| 2-19-647 | N-Biph | N-Biph | b2 | a6 | H |
| 2-19-648 | N-Biph | N-Biph | b3 | a6 | H |
| 2-19-649 | N-Biph | N-Biph | H | b1 | H |
| 2-19-650 | N-Biph | N-Biph | t-bu | b1 | H |
| 2-19-651 | N-Biph | N-Biph | methyl | b1 | H |
| 2-19-652 | N-Biph | N-Biph | b1 | b1 | H |
| 2-19-653 | N-Biph | N-Biph | b2 | b1 | H |
| 2-19-654 | N-Biph | N-Biph | b3 | b1 | H |
| 2-19-655 | N-Biph | N-Biph | H | b2 | H |
| 2-19-656 | N-Biph | N-Biph | t-bu | b2 | H |
| 2-19-657 | N-Biph | N-Biph | methyl | b2 | H |
| 2-19-658 | N-Biph | N-Biph | b1 | b2 | H |
| 2-19-659 | N-Biph | N-Biph | b2 | b2 | H |
| 2-19-660 | N-Biph | N-Biph | b3 | b2 | H |
| 2-19-661 | N-Biph | N-Biph | H | b3 | H |
| 2-19-662 | N-Biph | N-Biph | t-bu | b3 | H |
| 2-19-663 | N-Biph | N-Biph | methyl | b3 | H |
| 2-19-664 | N-Biph | N-Biph | b1 | b3 | H |
| 2-19-665 | N-Biph | N-Biph | b2 | b3 | H |
| 2-19-666 | N-Biph | N-Biph | b3 | b3 | H |
| 2-19-667 | N-Biph | N-Biph | H | b4 | H |
| 2-19-668 | N-Biph | N-Biph | t-bu | b4 | H |
| 2-19-669 | N-Biph | N-Biph | methyl | b4 | H |
| 2-19-670 | N-Biph | N-Biph | b1 | b4 | H |
| 2-19-671 | N-Biph | N-Biph | b2 | b4 | H |
| 2-19-672 | N-Biph | N-Biph | b3 | b4 | H |
| 2-19-673 | N-Biph | N-Biph | H | b5 | H |
| 2-19-674 | N-Biph | N-Biph | t-bu | b5 | H |
| 2-19-675 | N-Biph | N-Biph | methyl | b5 | H |
| 2-19-676 | N-Biph | N-Biph | b1 | b5 | H |
| 2-19-677 | N-Biph | N-Biph | b2 | b5 | H |
| 2-19-678 | N-Biph | N-Biph | b3 | b5 | H |
| 2-19-679 | N-Biph | N-Biph | H | b6 | H |
| 2-19-680 | N-Biph | N-Biph | t-bu | b6 | H |
| 2-19-681 | N-Biph | N-Biph | methyl | b6 | H |
| 2-19-682 | N-Biph | N-Biph | b1 | b6 | H |
| 2-19-683 | N-Biph | N-Biph | b2 | b6 | H |
| 2-19-684 | N-Biph | N-Biph | b3 | b6 | H |
| 2-19-685 | N-Biph | N-Biph | H | b7 | H |
| 2-19-686 | N-Biph | N-Biph | t-bu | b7 | H |
| 2-19-687 | N-Biph | N-Biph | methyl | b7 | H |
| 2-19-688 | N-Biph | N-Biph | b1 | b7 | H |
| 2-19-689 | N-Biph | N-Biph | b2 | b7 | H |
| 2-19-690 | N-Biph | N-Biph | b3 | b7 | H |
| 2-19-691 | N-Biph | N-Biph | H | b8 | H |
| 2-19-692 | N-Biph | N-Biph | t-bu | b8 | H |
| 2-19-693 | N-Biph | N-Biph | methyl | b8 | H |
| 2-19-694 | N-Biph | N-Biph | b1 | b8 | H |
| 2-19-695 | N-Biph | N-Biph | b2 | b8 | H |
| 2-19-696 | N-Biph | N-Biph | b3 | b8 | H |
| 2-19-697 | N-Biph | N-Biph | H | b9 | H |
| 2-19-698 | N-Biph | N-Biph | t-bu | b9 | H |
| 2-19-699 | N-Biph | N-Biph | methyl | b9 | H |
| 2-19-700 | N-Biph | N-Biph | b1 | b9 | H |
| 2-19-701 | N-Biph | N-Biph | b2 | b9 | H |
| 2-19-702 | N-Biph | N-Biph | b3 | b9 | H |
| 2-19-703 | N-Biph | N-Biph | H | b10 | H |
| 2-19-704 | N-Biph | N-Biph | t-bu | b10 | H |
| 2-19-705 | N-Biph | N-Biph | methyl | b10 | H |
| 2-19-706 | N-Biph | N-Biph | b1 | b10 | H |
| 2-19-707 | N-Biph | N-Biph | b2 | b10 | H |
| 2-19-708 | N-Biph | N-Biph | b3 | b10 | H |
| 2-19-709 | N-Biph | N-Biph | H | a1 | a1 |
| 2-19-710 | N-Biph | N-Biph | t-bu | a1 | a1 |
| 2-19-711 | N-Biph | N-Biph | methyl | a1 | a1 |
| 2-19-712 | N-Biph | N-Biph | b1 | a1 | a1 |
| 2-19-713 | N-Biph | N-Biph | b2 | a1 | a1 |
| 2-19-714 | N-Biph | N-Biph | b3 | a1 | a1 |
| 2-19-715 | N-Biph | N-Biph | H | a2 | a2 |
| 2-19-716 | N-Biph | N-Biph | t-bu | a2 | a2 |
| 2-19-717 | N-Biph | N-Biph | methyl | a2 | a2 |
| 2-19-718 | N-Biph | N-Biph | b1 | a2 | a2 |
| 2-19-719 | N-Biph | N-Biph | b2 | a2 | a2 |
| 2-19-720 | N-Biph | N-Biph | b3 | a2 | a2 |
| 2-19-721 | N-Biph | N-Biph | H | a3 | a3 |
| 2-19-722 | N-Biph | N-Biph | t-bu | a3 | a3 |
| 2-19-723 | N-Biph | N-Biph | methyl | a3 | a3 |
| 2-19-724 | N-Biph | N-Biph | b1 | a3 | a3 |
| 2-19-725 | N-Biph | N-Biph | b2 | a3 | a3 |
| 2-19-726 | N-Biph | N-Biph | b3 | a3 | a3 |
| 2-19-727 | N-Biph | N-Biph | H | b1 | b1 |
| 2-19-728 | N-Biph | N-Biph | t-bu | b1 | b1 |
| 2-19-729 | N-Biph | N-Biph | methyl | b1 | b1 |
| 2-19-730 | N-Biph | N-Biph | b1 | b1 | b1 |
| 2-19-731 | N-Biph | N-Biph | b2 | b1 | b1 |
| 2-19-732 | N-Biph | N-Biph | b3 | b1 | b1 |
| 2-19-733 | N-Biph | N-Biph | H | b2 | b2 |
| 2-19-734 | N-Biph | N-Biph | t-bu | b2 | b2 |
| 2-19-735 | N-Biph | N-Biph | methyl | b2 | b2 |
| 2-19-736 | N-Biph | N-Biph | b1 | b2 | b2 |
| 2-19-737 | N-Biph | N-Biph | b2 | b2 | b2 |
| 2-19-738 | N-Biph | N-Biph | b3 | b2 | b2 |
| 2-19-739 | N-Biph | N-Biph | H | b3 | b3 |
| 2-19-740 | N-Biph | N-Biph | t-bu | b3 | b3 |
| 2-19-741 | N-Biph | N-Biph | methyl | b3 | b3 |
| 2-19-742 | N-Biph | N-Biph | b1 | b3 | b3 |
| 2-19-743 | N-Biph | N-Biph | b2 | b3 | b3 |
| 2-19-744 | N-Biph | N-Biph | b3 | b3 | b3 |
| 2-19-745 | N-Biph | N-Biph | H | b5 | b5 |
| 2-19-746 | N-Biph | N-Biph | t-bu | b5 | b5 |
| 2-19-747 | N-Biph | N-Biph | methyl | b5 | b5 |
| 2-19-748 | N-Biph | N-Biph | b1 | b5 | b5 |
| 2-19-749 | N-Biph | N-Biph | b2 | b5 | b5 |
| 2-19-750 | N-Biph | N-Biph | b3 | b5 | b5 |
| 2-19-751 | N-Biph | N-Biph | H | b6 | b6 |
| 2-19-752 | N-Biph | N-Biph | t-bu | b6 | b6 |
| 2-19-753 | N-Biph | N-Biph | methyl | b6 | b6 |
| 2-19-754 | N-Biph | N-Biph | b1 | b6 | b6 |
| 2-19-755 | N-Biph | N-Biph | b2 | b6 | b6 |
| 2-19-756 | N-Biph | N-Biph | b3 | b6 | b6 |
| 2-19-757 | N-Biph | N-Biph | H | b7 | b7 |
| 2-19-758 | N-Biph | N-Biph | t-bu | b7 | b7 |
| 2-19-759 | N-Biph | N-Biph | methyl | b7 | b7 |
| 2-19-760 | N-Biph | N-Biph | b1 | b7 | b7 |
| 2-19-761 | N-Biph | N-Biph | b2 | b7 | b7 |
| 2-19-762 | N-Biph | N-Biph | b3 | b7 | b7 |
| 2-19-763 | O | N—Ph | H | H | H |
| 2-19-764 | O | N—Ph | t-bu | H | H |
| 2-19-765 | O | N—Ph | methyl | H | H |
| 2-19-766 | O | N—Ph | t-bu | t-bu | t-bu |
| 2-19-767 | O | N—Ph | methyl | methyl | methyl |
| 2-19-768 | O | N—Ph | b1 | H | H |
| 2-19-769 | O | N—Ph | b2 | H | H |
| 2-19-770 | O | N—Ph | b3 | H | H |
| 2-19-771 | O | N—Ph | H | H | a1 |
| 2-19-772 | O | N—Ph | t-bu | H | a1 |
| 2-19-773 | O | N—Ph | methyl | H | a1 |
| 2-19-774 | O | N—Ph | b1 | H | a1 |
| 2-19-775 | O | N—Ph | b2 | H | a1 |
| 2-19-776 | O | N—Ph | b3 | H | a1 |
| 2-19-777 | O | N—Ph | H | H | a2 |
| 2-19-778 | O | N—Ph | t-bu | H | a2 |
| 2-19-779 | O | N—Ph | methyl | H | a2 |
| 2-19-780 | O | N—Ph | b1 | H | a2 |
| 2-19-781 | O | N—Ph | b2 | H | a2 |
| 2-19-782 | O | N—Ph | b3 | H | a2 |
| 2-19-783 | O | N—Ph | H | H | a3 |
| 2-19-784 | O | N—Ph | t-bu | H | a3 |
| 2-19-785 | O | N—Ph | methyl | H | a3 |
| 2-19-786 | O | N—Ph | b1 | H | a3 |
| 2-19-787 | O | N—Ph | b2 | H | a3 |
| 2-19-788 | O | N—Ph | b3 | H | a3 |
| 2-19-789 | O | N—Ph | H | H | a4 |
| 2-19-790 | O | N—Ph | t-bu | H | a4 |
| 2-19-791 | O | N—Ph | methyl | H | a4 |
| 2-19-792 | O | N—Ph | b1 | H | a4 |
| 2-19-793 | O | N—Ph | b2 | H | a4 |
| 2-19-794 | O | N—Ph | b3 | H | a4 |
| 2-19-795 | O | N—Ph | H | H | a5 |
| 2-19-796 | O | N—Ph | t-bu | H | a5 |
| 2-19-797 | O | N—Ph | methyl | H | a5 |
| 2-19-798 | O | N—Ph | b1 | H | a5 |
| 2-19-799 | O | N—Ph | b2 | H | a5 |
| 2-19-800 | O | N—Ph | b3 | H | a5 |
| 2-19-801 | O | N—Ph | H | H | a6 |
| 2-19-802 | O | N—Ph | t-bu | H | a6 |
| 2-19-803 | O | N—Ph | methyl | H | a6 |
| 2-19-804 | O | N—Ph | b1 | H | a6 |
| 2-19-805 | O | N—Ph | b2 | H | a6 |
| 2-19-806 | O | N—Ph | b3 | H | a6 |
| 2-19-807 | O | N—Ph | H | H | b1 |
| 2-19-808 | O | N—Ph | t-bu | H | b1 |
| 2-19-809 | O | N—Ph | methyl | H | b1 |
| 2-19-810 | O | N—Ph | b1 | H | b1 |
| 2-19-811 | O | N—Ph | b2 | H | b1 |
| 2-19-812 | O | N—Ph | b3 | H | b1 |
| 2-19-813 | O | N—Ph | H | H | b2 |
| 2-19-814 | O | N—Ph | t-bu | H | b2 |
| 2-19-815 | O | N—Ph | methyl | H | b2 |
| 2-19-816 | O | N—Ph | b1 | H | b2 |
| 2-19-817 | O | N—Ph | b2 | H | b2 |
| 2-19-818 | O | N—Ph | b3 | H | b2 |
| 2-19-819 | O | N—Ph | H | H | b3 |
| 2-19-820 | O | N—Ph | t-bu | H | b3 |
| 2-19-821 | O | N—Ph | methyl | H | b3 |
| 2-19-822 | O | N—Ph | b1 | H | b3 |
| 2-19-823 | O | N—Ph | b2 | H | b3 |
| 2-19-824 | O | N—Ph | b3 | H | b3 |
| 2-19-825 | O | N—Ph | H | H | b4 |
| 2-19-826 | O | N—Ph | t-bu | H | b4 |
| 2-19-827 | O | N—Ph | methyl | H | b4 |
| 2-19-828 | O | N—Ph | b1 | H | b4 |
| 2-19-829 | O | N—Ph | b2 | H | b4 |
| 2-19-830 | O | N—Ph | b3 | H | b4 |
| 2-19-831 | O | N—Ph | H | H | b5 |
| 2-19-832 | O | N—Ph | t-bu | H | b5 |
| 2-19-833 | O | N—Ph | methyl | H | b5 |
| 2-19-834 | O | N—Ph | b1 | H | b5 |
| 2-19-835 | O | N—Ph | b2 | H | b5 |
| 2-19-836 | O | N—Ph | b3 | H | b5 |
| 2-19-837 | O | N—Ph | H | H | b6 |
| 2-19-838 | O | N—Ph | t-bu | H | b6 |
| 2-19-839 | O | N—Ph | methyl | H | b6 |
| 2-19-840 | O | N—Ph | b1 | H | b6 |
| 2-19-841 | O | N—Ph | b2 | H | b6 |
| 2-19-842 | O | N—Ph | b3 | H | b6 |
| 2-19-843 | O | N—Ph | H | H | b7 |
| 2-19-844 | O | N—Ph | t-bu | H | b7 |
| 2-19-845 | O | N—Ph | methyl | H | b7 |
| 2-19-846 | O | N—Ph | b1 | H | b7 |
| 2-19-847 | O | N—Ph | b2 | H | b7 |
| 2-19-848 | O | N—Ph | b3 | H | b7 |
| 2-19-849 | O | N—Ph | H | H | b8 |
| 2-19-850 | O | N—Ph | t-bu | H | b8 |
| 2-19-851 | O | N—Ph | methyl | H | b8 |
| 2-19-852 | O | N—Ph | b1 | H | b8 |
| 2-19-853 | O | N—Ph | b2 | H | b8 |
| 2-19-854 | O | N—Ph | b3 | H | b8 |
| 2-19-855 | O | N—Ph | H | H | b9 |
| 2-19-856 | O | N—Ph | t-bu | H | b9 |
| 2-19-857 | O | N—Ph | methyl | H | b9 |
| 2-19-858 | O | N—Ph | b1 | H | b9 |
| 2-19-859 | O | N—Ph | b2 | H | b9 |
| 2-19-860 | O | N—Ph | b3 | H | b9 |
| 2-19-861 | O | N—Ph | H | H | b10 |
| 2-19-862 | O | N—Ph | t-bu | H | b10 |
| 2-19-863 | O | N—Ph | methyl | H | b10 |
| 2-19-864 | O | N—Ph | b1 | H | b10 |
| 2-19-865 | O | N—Ph | b2 | H | b10 |
| 2-19-866 | O | N—Ph | b3 | H | b10 |
| 2-19-867 | O | N—Ph | H | a1 | H |
| 2-19-868 | O | N—Ph | t-bu | a1 | H |
| 2-19-869 | O | N—Ph | methyl | a1 | H |
| 2-19-870 | O | N—Ph | b1 | a1 | H |
| 2-19-871 | O | N—Ph | b2 | a1 | H |
| 2-19-872 | O | N—Ph | b3 | a1 | H |
| 2-19-873 | O | N—Ph | H | a2 | H |
| 2-19-874 | O | N—Ph | t-bu | a2 | H |
| 2-19-875 | O | N—Ph | methyl | a2 | H |
| 2-19-876 | O | N—Ph | b1 | a2 | H |
| 2-19-877 | O | N—Ph | b2 | a2 | H |
| 2-19-878 | O | N—Ph | b3 | a2 | H |
| 2-19-879 | O | N—Ph | H | a3 | H |
| 2-19-880 | O | N—Ph | t-bu | a3 | H |
| 2-19-881 | O | N—Ph | methyl | a3 | H |
| 2-19-882 | O | N—Ph | b1 | a3 | H |
| 2-19-883 | O | N—Ph | b2 | a3 | H |
| 2-19-884 | O | N—Ph | b3 | a3 | H |
| 2-19-885 | O | N—Ph | H | a4 | H |
| 2-19-886 | O | N—Ph | t-bu | a4 | H |
| 2-19-887 | O | N—Ph | methyl | a4 | H |
| 2-19-888 | O | N—Ph | b1 | a4 | H |
| 2-19-889 | O | N—Ph | b2 | a4 | H |
| 2-19-890 | O | N—Ph | b3 | a4 | H |
| 2-19-891 | O | N—Ph | H | a5 | H |
| 2-19-892 | O | N—Ph | t-bu | a5 | H |
| 2-19-893 | O | N—Ph | methyl | a5 | H |
| 2-19-894 | O | N—Ph | b1 | a5 | H |
| 2-19-895 | O | N—Ph | b2 | a5 | H |
| 2-19-896 | O | N—Ph | b3 | a5 | H |
| 2-19-897 | O | N—Ph | H | a6 | H |
| 2-19-898 | O | N—Ph | t-bu | a6 | H |
| 2-19-899 | O | N—Ph | methyl | a6 | H |
| 2-19-900 | O | N—Ph | b1 | a6 | H |
| 2-19-901 | O | N—Ph | b2 | a6 | H |
| 2-19-902 | O | N—Ph | b3 | a6 | H |
| 2-19-903 | O | N—Ph | H | b1 | H |
| 2-19-904 | O | N—Ph | t-bu | b1 | H |
| 2-19-905 | O | N—Ph | methyl | b1 | H |
| 2-19-906 | O | N—Ph | b1 | b1 | H |
| 2-19-907 | O | N—Ph | b2 | b1 | H |
| 2-19-908 | O | N—Ph | b3 | b1 | H |
| 2-19-909 | O | N—Ph | H | b2 | H |
| 2-19-910 | O | N—Ph | t-bu | b2 | H |
| 2-19-911 | O | N—Ph | methyl | b2 | H |
| 2-19-912 | O | N—Ph | b1 | b2 | H |
| 2-19-913 | O | N—Ph | b2 | b2 | H |
| 2-19-914 | O | N—Ph | b3 | b2 | H |
| 2-19-915 | O | N—Ph | H | b3 | H |
| 2-19-916 | O | N—Ph | t-bu | b3 | H |
| 2-19-917 | O | N—Ph | methyl | b3 | H |
| 2-19-918 | O | N—Ph | b1 | b3 | H |
| 2-19-919 | O | N—Ph | b2 | b3 | H |
| 2-19-920 | O | N—Ph | b3 | b3 | H |
| 2-19-921 | O | N—Ph | H | b4 | H |
| 2-19-922 | O | N—Ph | t-bu | b4 | H |
| 2-19-923 | O | N—Ph | methyl | b4 | H |
| 2-19-924 | O | N—Ph | b1 | b4 | H |
| 2-19-925 | O | N—Ph | b2 | b4 | H |
| 2-19-926 | O | N—Ph | b3 | b4 | H |
| 2-19-927 | O | N—Ph | H | b5 | H |
| 2-19-928 | O | N—Ph | t-bu | b5 | H |
| 2-19-929 | O | N—Ph | methyl | b5 | H |
| 2-19-930 | O | N—Ph | b1 | b5 | H |
| 2-19-931 | O | N—Ph | b2 | b5 | H |
| 2-19-932 | O | N—Ph | b3 | b5 | H |
| 2-19-933 | O | N—Ph | H | b6 | H |
| 2-19-934 | O | N—Ph | t-bu | b6 | H |
| 2-19-935 | O | N—Ph | methyl | b6 | H |
| 2-19-936 | O | N—Ph | b1 | b6 | H |
| 2-19-937 | O | N—Ph | b2 | b6 | H |
| 2-19-938 | O | N—Ph | b3 | b6 | H |
| 2-19-939 | O | N—Ph | H | b7 | H |
| 2-19-940 | O | N—Ph | t-bu | b7 | H |
| 2-19-941 | O | N—Ph | methyl | b7 | H |
| 2-19-942 | O | N—Ph | b1 | b7 | H |
| 2-19-943 | O | N—Ph | b2 | b7 | H |
| 2-19-944 | O | N—Ph | b3 | b7 | H |
| 2-19-945 | O | N—Ph | H | b8 | H |
| 2-19-946 | O | N—Ph | t-bu | b8 | H |
| 2-19-947 | O | N—Ph | methyl | b8 | H |
| 2-19-948 | O | N—Ph | b1 | b8 | H |
| 2-19-949 | O | N—Ph | b2 | b8 | H |
| 2-19-950 | O | N—Ph | b3 | b8 | H |
| 2-19-951 | O | N—Ph | H | b9 | H |
| 2-19-952 | O | N—Ph | t-bu | b9 | H |
| 2-19-953 | O | N—Ph | methyl | b9 | H |
| 2-19-954 | O | N—Ph | b1 | b9 | H |
| 2-19-955 | O | N—Ph | b2 | b9 | H |
| 2-19-956 | O | N—Ph | b3 | b9 | H |
| 2-19-957 | O | N—Ph | H | b10 | H |
| 2-19-958 | O | N—Ph | t-bu | b10 | H |
| 2-19-959 | O | N—Ph | methyl | b10 | H |
| 2-19-960 | O | N—Ph | b1 | b10 | H |
| 2-19-961 | O | N—Ph | b2 | b10 | H |
| 2-19-962 | O | N—Ph | b3 | b10 | H |
| 2-19-963 | O | N—Ph | H | a1 | a1 |
| 2-19-964 | O | N—Ph | t-bu | a1 | a1 |
| 2-19-965 | O | N—Ph | methyl | a1 | a1 |
| 2-19-966 | O | N—Ph | b1 | a1 | a1 |
| 2-19-967 | O | N—Ph | b2 | a1 | a1 |
| 2-19-968 | O | N—Ph | b3 | a1 | a1 |
| 2-19-969 | O | N—Ph | H | a2 | a2 |
| 2-19-970 | O | N—Ph | t-bu | a2 | a2 |
| 2-19-971 | O | N—Ph | methyl | a2 | a2 |
| 2-19-972 | O | N—Ph | b1 | a2 | a2 |
| 2-19-973 | O | N—Ph | b2 | a2 | a2 |
| 2-19-974 | O | N—Ph | b3 | a2 | a2 |
| 2-19-975 | O | N—Ph | H | a3 | a3 |
| 2-19-976 | O | N—Ph | t-bu | a3 | a3 |
| 2-19-977 | O | N—Ph | methyl | a3 | a3 |
| 2-19-978 | O | N—Ph | b1 | a3 | a3 |
| 2-19-979 | O | N—Ph | b2 | a3 | a3 |
| 2-19-980 | O | N—Ph | b3 | a3 | a3 |
| 2-19-981 | O | N—Ph | H | b1 | b1 |
| 2-19-982 | O | N—Ph | t-bu | b1 | b1 |
| 2-19-983 | O | N—Ph | methyl | b1 | b1 |
| 2-19-984 | O | N—Ph | b1 | b1 | b1 |
| 2-19-985 | O | N—Ph | b2 | b1 | b1 |
| 2-19-986 | O | N—Ph | b3 | b1 | b1 |
| 2-19-987 | O | N—Ph | H | b2 | b2 |
| 2-19-988 | O | N—Ph | t-bu | b2 | b2 |
| 2-19-989 | O | N—Ph | methyl | b2 | b2 |
| 2-19-990 | O | N—Ph | b1 | b2 | b2 |
| 2-19-991 | O | N—Ph | b2 | b2 | b2 |
| 2-19-992 | O | N—Ph | b3 | b2 | b2 |
| 2-19-993 | O | N—Ph | H | b3 | b3 |
| 2-19-994 | O | N—Ph | t-bu | b3 | b3 |
| 2-19-995 | O | N—Ph | methyl | b3 | b3 |
| 2-19-996 | O | N—Ph | b1 | b3 | b3 |
| 2-19-997 | O | N—Ph | b2 | b3 | b3 |
| 2-19-998 | O | N—Ph | b3 | b3 | b3 |
| 2-19-999 | O | N—Ph | H | b5 | b5 |
| 2-19-1000 | O | N—Ph | t-bu | b5 | b5 |
| 2-19-1001 | O | N—Ph | methyl | b5 | b5 |
| 2-19-1002 | O | N—Ph | b1 | b5 | b5 |
| 2-19-1003 | O | N—Ph | b2 | b5 | b5 |
| 2-19-1004 | O | N—Ph | b3 | b5 | b5 |
| 2-19-1005 | O | N—Ph | H | b6 | b6 |
| 2-19-1006 | O | N—Ph | t-bu | b6 | b6 |
| 2-19-1007 | O | N—Ph | methyl | b6 | b6 |
| 2-19-1008 | O | N—Ph | b1 | b6 | b6 |
| 2-19-1009 | O | N—Ph | b2 | b6 | b6 |
| 2-19-1010 | O | N—Ph | b3 | b6 | b6 |
| 2-19-1011 | O | N—Ph | H | b7 | b7 |
| 2-19-1012 | O | N—Ph | t-bu | b7 | b7 |
| 2-19-1013 | O | N—Ph | methyl | b7 | b7 |
| 2-19-1014 | O | N—Ph | b1 | b7 | b7 |
| 2-19-1015 | O | N—Ph | b2 | b7 | b7 |
| 2-19-1016 | O | N—Ph | b3 | b7 | b7 |
| 2-19-1017 | S | N—Ph | H | H | H |
| 2-19-1018 | S | N—Ph | t-bu | H | H |
| 2-19-1019 | S | N—Ph | methyl | H | H |
| 2-19-1020 | S | N—Ph | t-bu | t-bu | t-bu |
| 2-19-1021 | S | N—Ph | methyl | methyl | methyl |
| 2-19-1022 | S | N—Ph | b1 | H | H |
| 2-19-1023 | S | N—Ph | b2 | H | H |
| 2-19-1024 | S | N—Ph | b3 | H | H |
| 2-19-1025 | S | N—Ph | H | H | a1 |
| 2-19-1026 | S | N—Ph | t-bu | H | a1 |
| 2-19-1027 | S | N—Ph | methyl | H | a1 |
| 2-19-1028 | S | N—Ph | b1 | H | a1 |
| 2-19-1029 | S | N—Ph | b2 | H | a1 |
| 2-19-1030 | S | N—Ph | b3 | H | a1 |
| 2-19-1031 | S | N—Ph | H | H | a2 |
| 2-19-1032 | S | N—Ph | t-bu | H | a2 |
| 2-19-1033 | S | N—Ph | methyl | H | a2 |
| 2-19-1034 | S | N—Ph | b1 | H | a2 |
| 2-19-1035 | S | N—Ph | b2 | H | a2 |
| 2-19-1036 | S | N—Ph | b3 | H | a2 |
| 2-19-1037 | S | N—Ph | H | H | a3 |
| 2-19-1038 | S | N—Ph | t-bu | H | a3 |
| 2-19-1039 | S | N—Ph | methyl | H | a3 |
| 2-19-1040 | S | N—Ph | b1 | H | a3 |
| 2-19-1041 | S | N—Ph | b2 | H | a3 |
| 2-19-1042 | S | N—Ph | b3 | H | a3 |
| 2-19-1043 | S | N—Ph | H | H | a4 |
| 2-19-1044 | S | N—Ph | t-bu | H | a4 |
| 2-19-1045 | S | N—Ph | methyl | H | a4 |
| 2-19-1046 | S | N—Ph | b1 | H | a4 |
| 2-19-1047 | S | N—Ph | b2 | H | a4 |
| 2-19-1048 | S | N—Ph | b3 | H | a4 |
| 2-19-1049 | S | N—Ph | H | H | a5 |
| 2-19-1050 | S | N—Ph | t-bu | H | a5 |
| 2-19-1051 | S | N—Ph | methyl | H | a5 |
| 2-19-1052 | S | N—Ph | b1 | H | a5 |
| 2-19-1053 | S | N—Ph | b2 | H | a5 |
| 2-19-1054 | S | N—Ph | b3 | H | a5 |
| 2-19-1055 | S | N—Ph | H | H | a6 |
| 2-19-1056 | S | N—Ph | t-bu | H | a6 |
| 2-19-1057 | S | N—Ph | methyl | H | a6 |
| 2-19-1058 | S | N—Ph | b1 | H | a6 |
| 2-19-1059 | S | N—Ph | b2 | H | a6 |
| 2-19-1060 | S | N—Ph | b3 | H | a6 |
| 2-19-1061 | S | N—Ph | H | H | b1 |
| 2-19-1062 | S | N—Ph | t-bu | H | b1 |
| 2-19-1063 | S | N—Ph | methyl | H | b1 |
| 2-19-1064 | S | N—Ph | b1 | H | b1 |
| 2-19-1065 | S | N—Ph | b2 | H | b1 |
| 2-19-1066 | S | N—Ph | b3 | H | b1 |
| 2-19-1067 | S | N—Ph | H | H | b2 |
| 2-19-1068 | S | N—Ph | t-bu | H | b2 |
| 2-19-1069 | S | N—Ph | methyl | H | b2 |
| 2-19-1070 | S | N—Ph | b1 | H | b2 |
| 2-19-1071 | S | N—Ph | b2 | H | b2 |
| 2-19-1072 | S | N—Ph | b3 | H | b2 |
| 2-19-1073 | S | N—Ph | H | H | b3 |
| 2-19-1074 | S | N—Ph | t-bu | H | b3 |
| 2-19-1075 | S | N—Ph | methyl | H | b3 |
| 2-19-1076 | S | N—Ph | b1 | H | b3 |
| 2-19-1077 | S | N—Ph | b2 | H | b3 |
| 2-19-1078 | S | N—Ph | b3 | H | b3 |
| 2-19-1079 | S | N—Ph | H | H | b4 |
| 2-19-1080 | S | N—Ph | t-bu | H | b4 |
| 2-19-1081 | S | N—Ph | methyl | H | b4 |
| 2-19-1082 | S | N—Ph | b1 | H | b4 |
| 2-19-1083 | S | N—Ph | b2 | H | b4 |
| 2-19-1084 | S | N—Ph | b3 | H | b4 |
| 2-19-1085 | S | N—Ph | H | H | b5 |
| 2-19-1086 | S | N—Ph | t-bu | H | b5 |
| 2-19-1087 | S | N—Ph | methyl | H | b5 |
| 2-19-1088 | S | N—Ph | b1 | H | b5 |
| 2-19-1089 | S | N—Ph | b2 | H | b5 |
| 2-19-1090 | S | N—Ph | b3 | H | b5 |
| 2-19-1091 | S | N—Ph | H | H | b6 |
| 2-19-1092 | S | N—Ph | t-bu | H | b6 |
| 2-19-1093 | S | N—Ph | methyl | H | b6 |
| 2-19-1094 | S | N—Ph | b1 | H | b6 |
| 2-19-1095 | S | N—Ph | b2 | H | b6 |
| 2-19-1096 | S | N—Ph | b3 | H | b6 |
| 2-19-1097 | S | N—Ph | H | H | b7 |
| 2-19-1098 | S | N—Ph | t-bu | H | b7 |
| 2-19-1099 | S | N—Ph | methyl | H | b7 |
| 2-19-1100 | S | N—Ph | b1 | H | b7 |
| 2-19-1101 | S | N—Ph | b2 | H | b7 |
| 2-19-1102 | S | N—Ph | b3 | H | b7 |
| 2-19-1103 | S | N—Ph | H | H | b8 |
| 2-19-1104 | S | N—Ph | t-bu | H | b8 |
| 2-19-1105 | S | N—Ph | methyl | H | b8 |
| 2-19-1106 | S | N—Ph | b1 | H | b8 |
| 2-19-1107 | S | N—Ph | b2 | H | b8 |
| 2-19-1108 | S | N—Ph | b3 | H | b8 |
| 2-19-1109 | S | N—Ph | H | H | b9 |
| 2-19-1110 | S | N—Ph | t-bu | H | b9 |
| 2-19-1111 | S | N—Ph | methyl | H | b9 |
| 2-19-1112 | S | N—Ph | b1 | H | b9 |
| 2-19-1113 | S | N—Ph | b2 | H | b9 |
| 2-19-1114 | S | N—Ph | b3 | H | b9 |
| 2-19-1115 | S | N—Ph | H | H | b10 |
| 2-19-1116 | S | N—Ph | t-bu | H | b10 |
| 2-19-1117 | S | N—Ph | methyl | H | b10 |
| 2-19-1118 | S | N—Ph | b1 | H | b10 |
| 2-19-1119 | S | N—Ph | b2 | H | b10 |
| 2-19-1120 | S | N—Ph | b3 | H | b10 |
| 2-19-1121 | S | N—Ph | H | a1 | H |
| 2-19-1122 | S | N—Ph | t-bu | a1 | H |
| 2-19-1123 | S | N—Ph | methyl | a1 | H |
| 2-19-1124 | S | N—Ph | b1 | a1 | H |
| 2-19-1125 | S | N—Ph | b2 | a1 | H |
| 2-19-1126 | S | N—Ph | b3 | a1 | H |
| 2-19-1127 | S | N—Ph | H | a2 | H |
| 2-19-1128 | S | N—Ph | t-bu | a2 | H |
| 2-19-1129 | S | N—Ph | methyl | a2 | H |
| 2-19-1130 | S | N—Ph | b1 | a2 | H |
| 2-19-1131 | S | N—Ph | b2 | a2 | H |
| 2-19-1132 | S | N—Ph | b3 | a2 | H |
| 2-19-1133 | S | N—Ph | H | a3 | H |
| 2-19-1134 | S | N—Ph | t-bu | a3 | H |
| 2-19-1135 | S | N—Ph | methyl | a3 | H |
| 2-19-1136 | S | N—Ph | b1 | a3 | H |
| 2-19-1137 | S | N—Ph | b2 | a3 | H |
| 2-19-1138 | S | N—Ph | b3 | a3 | H |
| 2-19-1139 | S | N—Ph | H | a4 | H |
| 2-19-1140 | S | N—Ph | t-bu | a4 | H |
| 2-19-1141 | S | N—Ph | methyl | a4 | H |
| 2-19-1142 | S | N—Ph | b1 | a4 | H |
| 2-19-1143 | S | N—Ph | b2 | a4 | H |
| 2-19-1144 | S | N—Ph | b3 | a4 | H |
| 2-19-1145 | S | N—Ph | H | a5 | H |
| 2-19-1146 | S | N—Ph | t-bu | a5 | H |
| 2-19-1147 | S | N—Ph | methyl | a5 | H |
| 2-19-1148 | S | N—Ph | b1 | a5 | H |
| 2-19-1149 | S | N—Ph | b2 | a5 | H |
| 2-19-1150 | S | N—Ph | b3 | a5 | H |
| 2-19-1151 | S | N—Ph | H | a6 | H |
| 2-19-1152 | S | N—Ph | t-bu | a6 | H |
| 2-19-1153 | S | N—Ph | methyl | a6 | H |
| 2-19-1154 | S | N—Ph | b1 | a6 | H |
| 2-19-1155 | S | N—Ph | b2 | a6 | H |
| 2-19-1156 | S | N—Ph | b3 | a6 | H |
| 2-19-1157 | S | N—Ph | H | b1 | H |
| 2-19-1158 | S | N—Ph | t-bu | b1 | H |
| 2-19-1159 | S | N—Ph | methyl | b1 | H |
| 2-19-1160 | S | N—Ph | b1 | b1 | H |
| 2-19-1161 | S | N—Ph | b2 | b1 | H |
| 2-19-1162 | S | N—Ph | b3 | b1 | H |
| 2-19-1163 | S | N—Ph | H | b2 | H |
| 2-19-1164 | S | N—Ph | t-bu | b2 | H |
| 2-19-1165 | S | N—Ph | methyl | b2 | H |
| 2-19-1166 | S | N—Ph | b1 | b2 | H |
| 2-19-1167 | S | N—Ph | b2 | b2 | H |
| 2-19-1168 | S | N—Ph | b3 | b2 | H |
| 2-19-1169 | S | N—Ph | H | b3 | H |
| 2-19-1170 | S | N—Ph | t-bu | b3 | H |
| 2-19-1171 | S | N—Ph | methyl | b3 | H |
| 2-19-1172 | S | N—Ph | b1 | b3 | H |
| 2-19-1173 | S | N—Ph | b2 | b3 | H |
| 2-19-1174 | S | N—Ph | b3 | b3 | H |
| 2-19-1175 | S | N—Ph | H | b4 | H |
| 2-19-1176 | S | N—Ph | t-bu | b4 | H |
| 2-19-1177 | S | N—Ph | methyl | b4 | H |
| 2-19-1178 | S | N—Ph | b1 | b4 | H |
| 2-19-1179 | S | N—Ph | b2 | b4 | H |
| 2-19-1180 | S | N—Ph | b3 | b4 | H |
| 2-19-1181 | S | N—Ph | H | b5 | H |
| 2-19-1182 | S | N—Ph | t-bu | b5 | H |
| 2-19-1183 | S | N—Ph | methyl | b5 | H |
| 2-19-1184 | S | N—Ph | b1 | b5 | H |
| 2-19-1185 | S | N—Ph | b2 | b5 | H |
| 2-19-1186 | S | N—Ph | b3 | b5 | H |
| 2-19-1187 | S | N—Ph | H | b6 | H |
| 2-19-1188 | S | N—Ph | t-bu | b6 | H |
| 2-19-1189 | S | N—Ph | methyl | b6 | H |
| 2-19-1190 | S | N—Ph | b1 | b6 | H |
| 2-19-1191 | S | N—Ph | b2 | b6 | H |
| 2-19-1192 | S | N—Ph | b3 | b6 | H |
| 2-19-1193 | S | N—Ph | H | b7 | H |
| 2-19-1194 | S | N—Ph | t-bu | b7 | H |
| 2-19-1195 | S | N—Ph | methyl | b7 | H |
| 2-19-1196 | S | N—Ph | b1 | b7 | H |
| 2-19-1197 | S | N—Ph | b2 | b7 | H |
| 2-19-1198 | S | N—Ph | b3 | b7 | H |
| 2-19-1199 | S | N—Ph | H | b8 | H |
| 2-19-1200 | S | N—Ph | t-bu | b8 | H |
| 2-19-1201 | S | N—Ph | methyl | b8 | H |
| 2-19-1202 | S | N—Ph | b1 | b8 | H |
| 2-19-1203 | S | N—Ph | b2 | b8 | H |
| 2-19-1204 | S | N—Ph | b3 | b8 | H |
| 2-19-1205 | S | N—Ph | H | b9 | H |
| 2-19-1206 | S | N—Ph | t-bu | b9 | H |
| 2-19-1207 | S | N—Ph | methyl | b9 | H |
| 2-19-1208 | S | N—Ph | b1 | b9 | H |
| 2-19-1209 | S | N—Ph | b2 | b9 | H |
| 2-19-1210 | S | N—Ph | b3 | b9 | H |
| 2-19-1211 | S | N—Ph | H | b10 | H |
| 2-19-1212 | S | N—Ph | t-bu | b10 | H |
| 2-19-1213 | S | N—Ph | methyl | b10 | H |
| 2-19-1214 | S | N—Ph | b1 | b10 | H |
| 2-19-1215 | S | N—Ph | b2 | b10 | H |
| 2-19-1216 | S | N—Ph | b3 | b10 | H |
| 2-19-1217 | S | N—Ph | H | a1 | a1 |
| 2-19-1218 | S | N—Ph | t-bu | a1 | a1 |
| 2-19-1219 | S | N—Ph | methyl | a1 | a1 |
| 2-19-1220 | S | N—Ph | b1 | a1 | a1 |
| 2-19-1221 | S | N—Ph | b2 | a1 | a1 |
| 2-19-1222 | S | N—Ph | b3 | a1 | a1 |
| 2-19-1223 | S | N—Ph | H | a2 | a2 |
| 2-19-1224 | S | N—Ph | t-bu | a2 | a2 |
| 2-19-1225 | S | N—Ph | methyl | a2 | a2 |
| 2-19-1226 | S | N—Ph | b1 | a2 | a2 |
| 2-19-1227 | S | N—Ph | b2 | a2 | a2 |
| 2-19-1228 | S | N—Ph | b3 | a2 | a2 |
| 2-19-1229 | S | N—Ph | H | a3 | a3 |
| 2-19-1230 | S | N—Ph | t-bu | a3 | a3 |
| 2-19-1231 | S | N—Ph | methyl | a3 | a3 |
| 2-19-1232 | S | N—Ph | b1 | a3 | a3 |
| 2-19-1233 | S | N—Ph | b2 | a3 | a3 |
| 2-19-1234 | S | N—Ph | b3 | a3 | a3 |
| 2-19-1235 | S | N—Ph | H | b1 | b1 |
| 2-19-1236 | S | N—Ph | t-bu | b1 | b1 |
| 2-19-1237 | S | N—Ph | methyl | b1 | b1 |
| 2-19-1238 | S | N—Ph | b1 | b1 | b1 |
| 2-19-1239 | S | N—Ph | b2 | b1 | b1 |
| 2-19-1240 | S | N—Ph | b3 | b1 | b1 |
| 2-19-1241 | S | N—Ph | H | b2 | b2 |
| 2-19-1242 | S | N—Ph | t-bu | b2 | b2 |
| 2-19-1243 | S | N—Ph | methyl | b2 | b2 |
| 2-19-1244 | S | N—Ph | b1 | b2 | b2 |
| 2-19-1245 | S | N—Ph | b2 | b2 | b2 |
| 2-19-1246 | S | N—Ph | b3 | b2 | b2 |
| 2-19-1247 | S | N—Ph | H | b3 | b3 |
| 2-19-1248 | S | N—Ph | t-bu | b3 | b3 |
| 2-19-1249 | S | N—Ph | methyl | b3 | b3 |
| 2-19-1250 | S | N—Ph | b1 | b3 | b3 |
| 2-19-1251 | S | N—Ph | b2 | b3 | b3 |
| 2-19-1252 | S | N—Ph | b3 | b3 | b3 |
| 2-19-1253 | S | N—Ph | H | b5 | b5 |
| 2-19-1254 | S | N—Ph | t-bu | b5 | b5 |
| 2-19-1255 | S | N—Ph | methyl | b5 | b5 |
| 2-19-1256 | S | N—Ph | b1 | b5 | b5 |
| 2-19-1257 | S | N—Ph | b2 | b5 | b5 |
| 2-19-1258 | S | N—Ph | b3 | b5 | b5 |
| 2-19-1259 | S | N—Ph | H | b6 | b6 |
| 2-19-1260 | S | N—Ph | t-bu | b6 | b6 |
| 2-19-1261 | S | N—Ph | methyl | b6 | b6 |
| 2-19-1262 | S | N—Ph | b1 | b6 | b6 |
| 2-19-1263 | S | N—Ph | b2 | b6 | b6 |
| 2-19-1264 | S | N—Ph | b3 | b6 | b6 |
| 2-19-1265 | S | N—Ph | H | b7 | b7 |
| 2-19-1266 | S | N—Ph | t-bu | b7 | b7 |
| 2-19-1267 | S | N—Ph | methyl | b7 | b7 |
| 2-19-1268 | S | N—Ph | b1 | b7 | b7 |
| 2-19-1269 | S | N—Ph | b2 | b7 | b7 |
| 2-19-1270 | S | N—Ph | b3 | b7 | b7 |

Group 2-20
The compounds are represented by the formula 2-20 below, wherein CY₁ and CY₂ are each a benzene group, X₁ is B, and Y1, Y2, Y3, R3a, (R1)a1, (R2)a2 are each shown in the table 20 below, 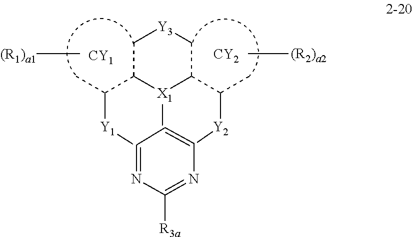
TABLE 20

| Compound | | | | | |
|---|---|---|---|---|---|
| No. | Y1/Y2 | Y3 | R3a | (R1)a1 | (R2)a2 |
| 2-20-001 | N-Ph | N-Ph | H | H | H |
| 2-20-002 | N-Ph | N-Ph | t-bu | H | H |
| 2-20-003 | N-Ph | N-Ph | methyl | H | H |
| 2-20-004 | N-Ph | N-Ph | t-bu | t-bu | t-bu |
| 2-20-005 | N-Ph | N-Ph | methyl | methyl | methyl |
| 2-20-006 | N-Ph | N-Ph | b1 | H | H |
| 2-20-007 | N-Ph | N-Ph | b2 | H | H |
| 2-20-008 | N-Ph | N-Ph | b3 | H | H |
| 2-20-009 | N-Ph | N-Ph | H | H | a1 |
| 2-20-010 | N-Ph | N-Ph | t-bu | H | a1 |
| 2-20-011 | N-Ph | N-Ph | methyl | H | a1 |
| 2-20-012 | N-Ph | N-Ph | b1 | H | a1 |
| 2-20-013 | N-Ph | N-Ph | b2 | H | a1 |
| 2-20-014 | N-Ph | N-Ph | b3 | H | a1 |
| 2-20-015 | N-Ph | N-Ph | H | H | a2 |
| 2-20-016 | N-Ph | N-Ph | t-bu | H | a2 |
| 2-20-017 | N-Ph | N-Ph | methyl | H | a2 |
| 2-20-018 | N-Ph | N-Ph | b1 | H | a2 |
| 2-20-019 | N-Ph | N-Ph | b2 | H | a2 |
| 2-20-020 | N-Ph | N-Ph | b3 | H | a2 |
| 2-20-021 | N-Ph | N-Ph | H | H | a3 |
| 2-20-022 | N-Ph | N-Ph | t-bu | H | a3 |
| 2-20-023 | N-Ph | N-Ph | methyl | H | a3 |
| 2-20-024 | N-Ph | N-Ph | b1 | H | a3 |
| 2-20-025 | N-Ph | N-Ph | b2 | H | a3 |
| 2-20-026 | N-Ph | N-Ph | b3 | H | a3 |
| 2-20-027 | N-Ph | N-Ph | H | H | a4 |
| 2-20-028 | N-Ph | N-Ph | t-bu | H | a4 |
| 2-20-029 | N-Ph | N-Ph | methyl | H | a4 |
| 2-20-030 | N-Ph | N-Ph | b1 | H | a4 |
| 2-20-031 | N-Ph | N-Ph | b2 | H | a4 |
| 2-20-032 | N-Ph | N-Ph | b3 | H | a4 |
| 2-20-033 | N-Ph | N-Ph | H | H | a5 |
| 2-20-034 | N-Ph | N-Ph | t-bu | H | a5 |
| 2-20-035 | N-Ph | N-Ph | methyl | H | a5 |
| 2-20-036 | N-Ph | N-Ph | b1 | H | a5 |
| 2-20-037 | N-Ph | N-Ph | b2 | H | a5 |
| 2-20-038 | N-Ph | N-Ph | b3 | H | a5 |
| 2-20-039 | N-Ph | N-Ph | H | H | a6 |
| 2-20-040 | N-Ph | N-Ph | t-bu | H | a6 |
| 2-20-041 | N-Ph | N-Ph | methyl | H | a6 |
| 2-20-042 | N-Ph | N-Ph | b1 | H | a6 |
| 2-20-043 | N-Ph | N-Ph | b2 | H | a6 |
| 2-20-044 | N-Ph | N-Ph | b3 | H | a6 |
| 2-20-045 | N-Ph | N-Ph | H | H | b1 |
| 2-20-046 | N-Ph | N-Ph | t-bu | H | b1 |
| 2-20-047 | N-Ph | N-Ph | methyl | H | b1 |
| 2-20-048 | N-Ph | N-Ph | b1 | H | b1 |
| 2-20-049 | N-Ph | N-Ph | b2 | H | b1 |
| 2-20-050 | N-Ph | N-Ph | b3 | H | b1 |
| 2-20-051 | N-Ph | N-Ph | H | H | b2 |
| 2-20-052 | N-Ph | N-Ph | t-bu | H | b2 |
| 2-20-053 | N-Ph | N-Ph | methyl | H | b2 |
| 2-20-054 | N-Ph | N-Ph | b1 | H | b2 |
| 2-20-055 | N-Ph | N-Ph | b2 | H | b2 |
| 2-20-056 | N-Ph | N-Ph | b3 | H | b2 |
| 2-20-057 | N-Ph | N-Ph | H | H | b3 |
| 2-20-058 | N-Ph | N-Ph | t-bu | H | b3 |
| 2-20-059 | N-Ph | N-Ph | methyl | H | b3 |
| 2-20-060 | N-Ph | N-Ph | b1 | H | b3 |
| 2-20-061 | N-Ph | N-Ph | b2 | H | b3 |
| 2-20-062 | N-Ph | N-Ph | b3 | H | b3 |
| 2-20-063 | N-Ph | N-Ph | H | H | b4 |
| 2-20-064 | N-Ph | N-Ph | t-bu | H | b4 |
| 2-20-065 | N-Ph | N-Ph | methyl | H | b4 |
| 2-20-066 | N-Ph | N-Ph | b1 | H | b4 |
| 2-20-067 | N-Ph | N-Ph | b2 | H | b4 |
| 2-20-068 | N-Ph | N-Ph | b3 | H | b4 |
| 2-20-069 | N-Ph | N-Ph | H | H | b5 |
| 2-20-070 | N-Ph | N-Ph | t-bu | H | b5 |
| 2-20-071 | N-Ph | N-Ph | methyl | H | b5 |
| 2-20-072 | N-Ph | N-Ph | b1 | H | b5 |
| 2-20-073 | N-Ph | N-Ph | b2 | H | b5 |
| 2-20-074 | N-Ph | N-Ph | b3 | H | b5 |
| 2-20-075 | N-Ph | N-Ph | H | H | b6 |
| 2-20-076 | N-Ph | N-Ph | t-bu | H | b6 |
| 2-20-077 | N-Ph | N-Ph | methyl | H | b6 |
| 2-20-078 | N-Ph | N-Ph | b1 | H | b6 |
| 2-20-079 | N-Ph | N-Ph | b2 | H | b6 |
| 2-20-080 | N-Ph | N-Ph | b3 | H | b6 |
| 2-20-081 | N-Ph | N-Ph | H | H | b7 |
| 2-20-082 | N-Ph | N-Ph | t-bu | H | b7 |
| 2-20-083 | N-Ph | N-Ph | methyl | H | b7 |
| 2-20-084 | N-Ph | N-Ph | b1 | H | b7 |
| 2-20-085 | N-Ph | N-Ph | b2 | H | b7 |
| 2-20-086 | N-Ph | N-Ph | b3 | H | b7 |
| 2-20-087 | N-Ph | N-Ph | H | H | b8 |
| 2-20-088 | N-Ph | N-Ph | t-bu | H | b8 |
| 2-20-089 | N-Ph | N-Ph | methyl | H | b8 |
| 2-20-090 | N-Ph | N-Ph | b1 | H | b8 |
| 2-20-091 | N-Ph | N-Ph | b2 | H | b8 |
| 2-20-092 | N-Ph | N-Ph | b3 | H | b8 |
| 2-20-093 | N-Ph | N-Ph | H | H | b9 |
| 2-20-094 | N-Ph | N-Ph | t-bu | H | b9 |
| 2-20-095 | N-Ph | N-Ph | methyl | H | b9 |
| 2-20-096 | N-Ph | N-Ph | b1 | H | b9 |
| 2-20-097 | N-Ph | N-Ph | b2 | H | b9 |
| 2-20-098 | N-Ph | N-Ph | b3 | H | b9 |
| 2-20-099 | N-Ph | N-Ph | H | H | b10 |
| 2-20-100 | N-Ph | N-Ph | t-bu | H | b10 |
| 2-20-101 | N-Ph | N-Ph | methyl | H | b10 |
| 2-20-102 | N-Ph | N-Ph | b1 | H | b10 |
| 2-20-103 | N-Ph | N-Ph | b2 | H | b10 |
| 2-20-104 | N-Ph | N-Ph | b3 | H | b10 |
| 2-20-105 | N-Ph | N-Ph | H | a1 | H |
| 2-20-106 | N-Ph | N-Ph | t-bu | a1 | H |
| 2-20-107 | N-Ph | N-Ph | methyl | a1 | H |
| 2-20-108 | N-Ph | N-Ph | b1 | a1 | H |
| 2-20-109 | N-Ph | N-Ph | b2 | a1 | H |
| 2-20-110 | N-Ph | N-Ph | b3 | a1 | H |
| 2-20-111 | N-Ph | N-Ph | H | a2 | H |
| 2-20-112 | N-Ph | N-Ph | t-bu | a2 | H |
| 2-20-113 | N-Ph | N-Ph | methyl | a2 | H |
| 2-20-114 | N-Ph | N-Ph | b1 | a2 | H |
| 2-20-115 | N-Ph | N-Ph | b2 | a2 | H |
| 2-20-116 | N-Ph | N-Ph | b3 | a2 | H |
| 2-20-117 | N-Ph | N-Ph | H | a3 | H |
| 2-20-118 | N-Ph | N-Ph | t-bu | a3 | H |
| 2-20-119 | N-Ph | N-Ph | methyl | a3 | H |
| 2-20-120 | N-Ph | N-Ph | b1 | a3 | H |
| 2-20-121 | N-Ph | N-Ph | b2 | a3 | H |
| 2-20-122 | N-Ph | N-Ph | b3 | a3 | H |
| 2-20-123 | N-Ph | N-Ph | H | a4 | H |
| 2-20-124 | N-Ph | N-Ph | t-bu | a4 | H |
| 2-20-125 | N-Ph | N-Ph | methyl | a4 | H |
| 2-20-126 | N-Ph | N-Ph | b1 | a4 | H |
| 2-20-127 | N-Ph | N-Ph | b2 | a4 | H |
| 2-20-128 | N-Ph | N-Ph | b3 | a4 | H |
| 2-20-129 | N-Ph | N-Ph | H | a5 | H |
| 2-20-130 | N-Ph | N-Ph | t-bu | a5 | H |
| 2-20-131 | N-Ph | N-Ph | methyl | a5 | H |
| 2-20-132 | N-Ph | N-Ph | b1 | a5 | H |
| 2-20-133 | N-Ph | N-Ph | b2 | a5 | H |
| 2-20-134 | N-Ph | N-Ph | b3 | a5 | H |
| 2-20-135 | N-Ph | N-Ph | H | a6 | H |
| 2-20-136 | N-Ph | N-Ph | t-bu | a6 | H |
| 2-20-137 | N-Ph | N-Ph | methyl | a6 | H |
| 2-20-138 | N-Ph | N-Ph | b1 | a6 | H |
| 2-20-139 | N-Ph | N-Ph | b2 | a6 | H |
| 2-20-140 | N-Ph | N-Ph | b3 | a6 | H |
| 2-20-141 | N-Ph | N-Ph | H | b1 | H |
| 2-20-142 | N-Ph | N-Ph | t-bu | b1 | H |
| 2-20-143 | N-Ph | N-Ph | methyl | b1 | H |
| 2-20-144 | N-Ph | N-Ph | b1 | b1 | H |
| 2-20-145 | N-Ph | N-Ph | b2 | b1 | H |
| 2-20-146 | N-Ph | N-Ph | b3 | b1 | H |
| 2-20-147 | N-Ph | N-Ph | H | b2 | H |
| 2-20-148 | N-Ph | N-Ph | t-bu | b2 | H |
| 2-20-149 | N-Ph | N-Ph | methyl | b2 | H |
| 2-20-150 | N-Ph | N-Ph | b1 | b2 | H |
| 2-20-151 | N-Ph | N-Ph | b2 | b2 | H |
| 2-20-152 | N-Ph | N-Ph | b3 | b2 | H |
| 2-20-153 | N-Ph | N-Ph | H | b3 | H |
| 2-20-154 | N-Ph | N-Ph | t-bu | b3 | H |
| 2-20-155 | N-Ph | N-Ph | methyl | b3 | H |
| 2-20-156 | N-Ph | N-Ph | b1 | b3 | H |
| 2-20-157 | N-Ph | N-Ph | b2 | b3 | H |
| 2-20-158 | N-Ph | N-Ph | b3 | b3 | H |
| 2-20-159 | N-Ph | N-Ph | H | b4 | H |
| 2-20-160 | N-Ph | N-Ph | t-bu | b4 | H |
| 2-20-161 | N-Ph | N-Ph | methyl | b4 | H |
| 2-20-162 | N-Ph | N-Ph | b1 | b4 | H |
| 2-20-163 | N-Ph | N-Ph | b2 | b4 | H |
| 2-20-164 | N-Ph | N-Ph | b3 | b4 | H |
| 2-20-165 | N-Ph | N-Ph | H | b5 | H |
| 2-20-166 | N-Ph | N-Ph | t-bu | b5 | H |
| 2-20-167 | N-Ph | N-Ph | methyl | b5 | H |
| 2-20-168 | N-Ph | N-Ph | b1 | b5 | H |
| 2-20-169 | N-Ph | N-Ph | b2 | b5 | H |
| 2-20-170 | N-Ph | N-Ph | b3 | b5 | H |
| 2-20-171 | N-Ph | N-Ph | H | b6 | H |
| 2-20-172 | N-Ph | N-Ph | t-bu | b6 | H |
| 2-20-173 | N-Ph | N-Ph | methyl | b6 | H |
| 2-20-174 | N-Ph | N-Ph | b1 | b6 | H |
| 2-20-175 | N-Ph | N-Ph | b2 | b6 | H |
| 2-20-176 | N-Ph | N-Ph | b3 | b6 | H |
| 2-20-177 | N-Ph | N-Ph | H | b7 | H |
| 2-20-178 | N-Ph | N-Ph | t-bu | b7 | H |
| 2-20-179 | N-Ph | N-Ph | methyl | b7 | H |
| 2-20-180 | N-Ph | N-Ph | b1 | b7 | H |
| 2-20-181 | N-Ph | N-Ph | b2 | b7 | H |
| 2-20-182 | N-Ph | N-Ph | b3 | b7 | H |
| 2-20-183 | N-Ph | N-Ph | H | b8 | H |
| 2-20-184 | N-Ph | N-Ph | t-bu | b8 | H |
| 2-20-185 | N-Ph | N-Ph | methyl | b8 | H |
| 2-20-186 | N-Ph | N-Ph | b1 | b8 | H |
| 2-20-187 | N-Ph | N-Ph | b2 | b8 | H |
| 2-20-188 | N-Ph | N-Ph | b3 | b8 | H |
| 2-20-189 | N-Ph | N-Ph | H | b9 | H |
| 2-20-190 | N-Ph | N-Ph | t-bu | b9 | H |
| 2-20-191 | N-Ph | N-Ph | methyl | b9 | H |
| 2-20-192 | N-Ph | N-Ph | b1 | b9 | H |
| 2-20-193 | N-Ph | N-Ph | b2 | b9 | H |
| 2-20-194 | N-Ph | N-Ph | b3 | b9 | H |
| 2-20-195 | N-Ph | N-Ph | H | b10 | H |
| 2-20-196 | N-Ph | N-Ph | t-bu | b10 | H |
| 2-20-197 | N-Ph | N-Ph | methyl | b10 | H |
| 2-20-198 | N-Ph | N-Ph | b1 | b10 | H |
| 2-20-199 | N-Ph | N-Ph | b2 | b10 | H |
| 2-20-200 | N-Ph | N-Ph | b3 | b10 | H |
| 2-20-201 | N-Ph | N-Ph | H | a1 | a1 |
| 2-20-202 | N-Ph | N-Ph | t-bu | a1 | a1 |
| 2-20-203 | N-Ph | N-Ph | methyl | a1 | a1 |
| 2-20-204 | N-Ph | N-Ph | b1 | a1 | a1 |
| 2-20-205 | N-Ph | N-Ph | b2 | a1 | a1 |
| 2-20-206 | N-Ph | N-Ph | b3 | a1 | a1 |
| 2-20-207 | N-Ph | N-Ph | H | a2 | a2 |
| 2-20-208 | N-Ph | N-Ph | t-bu | a2 | a2 |
| 2-20-209 | N-Ph | N-Ph | methyl | a2 | a2 |
| 2-20-210 | N-Ph | N-Ph | b1 | a2 | a2 |
| 2-20-211 | N-Ph | N-Ph | b2 | a2 | a2 |
| 2-20-212 | N-Ph | N-Ph | b3 | a2 | a2 |
| 2-20-213 | N-Ph | N-Ph | H | a3 | a3 |
| 2-20-214 | N-Ph | N-Ph | t-bu | a3 | a3 |
| 2-20-215 | N-Ph | N-Ph | methyl | a3 | a3 |
| 2-20-216 | N-Ph | N-Ph | b1 | a3 | a3 |
| 2-20-217 | N-Ph | N-Ph | b2 | a3 | a3 |
| 2-20-218 | N-Ph | N-Ph | b3 | a3 | a3 |
| 2-20-219 | N-Ph | N-Ph | H | b1 | b1 |
| 2-20-220 | N-Ph | N-Ph | t-bu | b1 | b1 |
| 2-20-221 | N-Ph | N-Ph | methyl | b1 | b1 |
| 2-20-222 | N-Ph | N-Ph | b1 | b1 | b1 |
| 2-20-223 | N-Ph | N-Ph | b2 | b1 | b1 |
| 2-20-224 | N-Ph | N-Ph | b3 | b1 | b1 |
| 2-20-225 | N-Ph | N-Ph | H | b2 | b2 |
| 2-20-226 | N-Ph | N-Ph | t-bu | b2 | b2 |
| 2-20-227 | N-Ph | N-Ph | methyl | b2 | b2 |
| 2-20-228 | N-Ph | N-Ph | b1 | b2 | b2 |
| 2-20-229 | N-Ph | N-Ph | b2 | b2 | b2 |
| 2-20-230 | N-Ph | N-Ph | b3 | b2 | b2 |
| 2-20-231 | N-Ph | N-Ph | H | b3 | b3 |
| 2-20-232 | N-Ph | N-Ph | t-bu | b3 | b3 |
| 2-20-233 | N-Ph | N-Ph | methyl | b3 | b3 |
| 2-20-234 | N-Ph | N-Ph | b1 | b3 | b3 |
| 2-20-235 | N-Ph | N-Ph | b2 | b3 | b3 |
| 2-20-236 | N-Ph | N-Ph | b3 | b3 | b3 |
| 2-20-237 | N-Ph | N-Ph | H | b5 | b5 |
| 2-20-238 | N-Ph | N-Ph | t-bu | b5 | b5 |
| 2-20-239 | N-Ph | N-Ph | methyl | b5 | b5 |
| 2-20-240 | N-Ph | N-Ph | b1 | b5 | b5 |
| 2-20-241 | N-Ph | N-Ph | b2 | b5 | b5 |
| 2-20-242 | N-Ph | N-Ph | b3 | b5 | b5 |
| 2-20-243 | N-Ph | N-Ph | H | b6 | b6 |
| 2-20-244 | N-Ph | N-Ph | t-bu | b6 | b6 |
| 2-20-245 | N-Ph | N-Ph | methyl | b6 | b6 |
| 2-20-246 | N-Ph | N-Ph | b1 | b6 | b6 |
| 2-20-247 | N-Ph | N-Ph | b2 | b6 | b6 |
| 2-20-248 | N-Ph | N-Ph | b3 | b6 | b6 |
| 2-20-249 | N-Ph | N-Ph | H | b7 | b7 |
| 2-20-250 | N-Ph | N-Ph | t-bu | b7 | b7 |
| 2-20-251 | N-Ph | N-Ph | methyl | b7 | b7 |
| 2-20-252 | N-Ph | N-Ph | b1 | b7 | b7 |
| 2-20-253 | N-Ph | N-Ph | b2 | b7 | b7 |
| 2-20-254 | N-Ph | N-Ph | b3 | b7 | b7 |
| 2-20-255 | N-tolyl | N-tolyl | H | H | H |
| 2-20-256 | N-tolyl | N-tolyl | t-bu | H | H |
| 2-20-257 | N-tolyl | N-tolyl | methyl | H | H |
| 2-20-258 | N-tolyl | N-tolyl | t-bu | t-bu | t-bu |
| 2-20-259 | N-tolyl | N-tolyl | methyl | methyl | methyl |
| 2-20-260 | N-tolyl | N-tolyl | b1 | H | H |
| 2-20-261 | N-tolyl | N-tolyl | b2 | H | H |
| 2-20-262 | N-tolyl | N-tolyl | b3 | H | H |
| 2-20-263 | N-tolyl | N-tolyl | H | H | a1 |
| 2-20-264 | N-tolyl | N-tolyl | t-bu | H | a1 |
| 2-20-265 | N-tolyl | N-tolyl | methyl | H | a1 |
| 2-20-266 | N-tolyl | N-tolyl | b1 | H | a1 |
| 2-20-267 | N-tolyl | N-tolyl | b2 | H | a1 |
| 2-20-268 | N-tolyl | N-tolyl | b3 | H | a1 |
| 2-20-269 | N-tolyl | N-tolyl | H | H | a2 |
| 2-20-270 | N-tolyl | N-tolyl | t-bu | H | a2 |
| 2-20-271 | N-tolyl | N-tolyl | methyl | H | a2 |
| 2-20-272 | N-tolyl | N-tolyl | b1 | H | a2 |
| 2-20-273 | N-tolyl | N-tolyl | b2 | H | a2 |
| 2-20-274 | N-tolyl | N-tolyl | b3 | H | a2 |
| 2-20-275 | N-tolyl | N-tolyl | H | H | a3 |
| 2-20-276 | N-tolyl | N-tolyl | t-bu | H | a3 |
| 2-20-277 | N-tolyl | N-tolyl | methyl | H | a3 |
| 2-20-278 | N-tolyl | N-tolyl | b1 | H | a3 |
| 2-20-279 | N-tolyl | N-tolyl | b2 | H | a3 |
| 2-20-280 | N-tolyl | N-tolyl | b3 | H | a3 |
| 2-20-281 | N-tolyl | N-tolyl | H | H | a4 |
| 2-20-282 | N-tolyl | N-tolyl | t-bu | H | a4 |
| 2-20-283 | N-tolyl | N-tolyl | methyl | H | a4 |
| 2-20-284 | N-tolyl | N-tolyl | b1 | H | a4 |
| 2-20-285 | N-tolyl | N-tolyl | b2 | H | a4 |
| 2-20-286 | N-tolyl | N-tolyl | b3 | H | a4 |
| 2-20-287 | N-tolyl | N-tolyl | H | H | a5 |
| 2-20-288 | N-tolyl | N-tolyl | t-bu | H | a5 |
| 2-20-289 | N-tolyl | N-tolyl | methyl | H | a5 |
| 2-20-290 | N-tolyl | N-tolyl | b1 | H | a5 |
| 2-20-291 | N-tolyl | N-tolyl | b2 | H | a5 |
| 2-20-292 | N-tolyl | N-tolyl | b3 | H | a5 |
| 2-20-293 | N-tolyl | N-tolyl | H | H | a6 |
| 2-20-294 | N-tolyl | N-tolyl | t-bu | H | a6 |
| 2-20-295 | N-tolyl | N-tolyl | methyl | H | a6 |
| 2-20-296 | N-tolyl | N-tolyl | b1 | H | a6 |
| 2-20-297 | N-tolyl | N-tolyl | b2 | H | a6 |
| 2-20-298 | N-tolyl | N-tolyl | b3 | H | a6 |
| 2-20-299 | N-tolyl | N-tolyl | H | H | b1 |
| 2-20-300 | N-tolyl | N-tolyl | t-bu | H | b1 |
| 2-20-301 | N-tolyl | N-tolyl | methyl | H | b1 |
| 2-20-302 | N-tolyl | N-tolyl | b1 | H | b1 |
| 2-20-303 | N-tolyl | N-tolyl | b2 | H | b1 |
| 2-20-304 | N-tolyl | N-tolyl | b3 | H | b1 |
| 2-20-305 | N-tolyl | N-tolyl | H | H | b2 |
| 2-20-306 | N-tolyl | N-tolyl | t-bu | H | b2 |
| 2-20-307 | N-tolyl | N-tolyl | methyl | H | b2 |
| 2-20-308 | N-tolyl | N-tolyl | b1 | H | b2 |
| 2-20-309 | N-tolyl | N-tolyl | b2 | H | b2 |
| 2-20-310 | N-tolyl | N-tolyl | b3 | H | b2 |
| 2-20-311 | N-tolyl | N-tolyl | H | H | b3 |
| 2-20-312 | N-tolyl | N-tolyl | t-bu | H | b3 |
| 2-20-313 | N-tolyl | N-tolyl | methyl | H | b3 |
| 2-20-314 | N-tolyl | N-tolyl | b1 | H | b3 |
| 2-20-315 | N-tolyl | N-tolyl | b2 | H | b3 |
| 2-20-316 | N-tolyl | N-tolyl | b3 | H | b3 |
| 2-20-317 | N-tolyl | N-tolyl | H | H | b4 |
| 2-20-318 | N-tolyl | N-tolyl | t-bu | H | b4 |
| 2-20-319 | N-tolyl | N-tolyl | methyl | H | b4 |
| 2-20-320 | N-tolyl | N-tolyl | b1 | H | b4 |
| 2-20-321 | N-tolyl | N-tolyl | b2 | H | b4 |
| 2-20-322 | N-tolyl | N-tolyl | b3 | H | b4 |
| 2-20-323 | N-tolyl | N-tolyl | H | H | b5 |
| 2-20-324 | N-tolyl | N-tolyl | t-bu | H | b5 |
| 2-20-325 | N-tolyl | N-tolyl | methyl | H | b5 |
| 2-20-326 | N-tolyl | N-tolyl | b1 | H | b5 |
| 2-20-327 | N-tolyl | N-tolyl | b2 | H | b5 |
| 2-20-328 | N-tolyl | N-tolyl | b3 | H | b5 |
| 2-20-329 | N-tolyl | N-tolyl | H | H | b6 |
| 2-20-330 | N-tolyl | N-tolyl | t-bu | H | b6 |
| 2-20-331 | N-tolyl | N-tolyl | methyl | H | b6 |
| 2-20-332 | N-tolyl | N-tolyl | b1 | H | b6 |
| 2-20-333 | N-tolyl | N-tolyl | b2 | H | b6 |
| 2-20-334 | N-tolyl | N-tolyl | b3 | H | b6 |
| 2-20-335 | N-tolyl | N-tolyl | H | H | b7 |
| 2-20-336 | N-tolyl | N-tolyl | t-bu | H | b7 |
| 2-20-337 | N-tolyl | N-tolyl | methyl | H | b7 |
| 2-20-338 | N-tolyl | N-tolyl | b1 | H | b7 |
| 2-20-339 | N-tolyl | N-tolyl | b2 | H | b7 |
| 2-20-340 | N-tolyl | N-tolyl | b3 | H | b7 |
| 2-20-341 | N-tolyl | N-tolyl | H | H | b8 |
| 2-20-342 | N-tolyl | N-tolyl | t-bu | H | b8 |
| 2-20-343 | N-tolyl | N-tolyl | methyl | H | b8 |
| 2-20-344 | N-tolyl | N-tolyl | b1 | H | b8 |
| 2-20-345 | N-tolyl | N-tolyl | b2 | H | b8 |
| 2-20-346 | N-tolyl | N-tolyl | b3 | H | b8 |
| 2-20-347 | N-tolyl | N-tolyl | H | H | b9 |
| 2-20-348 | N-tolyl | N-tolyl | t-bu | H | b9 |
| 2-20-349 | N-tolyl | N-tolyl | methyl | H | b9 |
| 2-20-350 | N-tolyl | N-tolyl | b1 | H | b9 |
| 2-20-351 | N-tolyl | N-tolyl | b2 | H | b9 |
| 2-20-352 | N-tolyl | N-tolyl | b3 | H | b9 |
| 2-20-353 | N-tolyl | N-tolyl | H | H | b10 |
| 2-20-354 | N-tolyl | N-tolyl | t-bu | H | b10 |
| 2-20-355 | N-tolyl | N-tolyl | methyl | H | b10 |
| 2-20-356 | N-tolyl | N-tolyl | b1 | H | b10 |
| 2-20-357 | N-tolyl | N-tolyl | b2 | H | b10 |
| 2-20-358 | N-tolyl | N-tolyl | b3 | H | b10 |
| 2-20-359 | N-tolyl | N-tolyl | H | a1 | H |
| 2-20-360 | N-tolyl | N-tolyl | t-bu | a1 | H |
| 2-20-361 | N-tolyl | N-tolyl | methyl | a1 | H |
| 2-20-362 | N-tolyl | N-tolyl | b1 | a1 | H |
| 2-20-363 | N-tolyl | N-tolyl | b2 | a1 | H |
| 2-20-364 | N-tolyl | N-tolyl | b3 | a1 | H |
| 2-20-365 | N-tolyl | N-tolyl | H | a2 | H |
| 2-20-366 | N-tolyl | N-tolyl | t-bu | a2 | H |
| 2-20-367 | N-tolyl | N-tolyl | methyl | a2 | H |
| 2-20-368 | N-tolyl | N-tolyl | b1 | a2 | H |
| 2-20-369 | N-tolyl | N-tolyl | b2 | a2 | H |
| 2-20-370 | N-tolyl | N-tolyl | b3 | a2 | H |
| 2-20-371 | N-tolyl | N-tolyl | H | a3 | H |
| 2-20-372 | N-tolyl | N-tolyl | t-bu | a3 | H |
| 2-20-373 | N-tolyl | N-tolyl | methyl | a3 | H |
| 2-20-374 | N-tolyl | N-tolyl | b1 | a3 | H |
| 2-20-375 | N-tolyl | N-tolyl | b2 | a3 | H |
| 2-20-376 | N-tolyl | N-tolyl | b3 | a3 | H |
| 2-20-377 | N-tolyl | N-tolyl | H | a4 | H |
| 2-20-378 | N-tolyl | N-tolyl | t-bu | a4 | H |
| 2-20-379 | N-tolyl | N-tolyl | methyl | a4 | H |
| 2-20-380 | N-tolyl | N-tolyl | b1 | a4 | H |
| 2-20-381 | N-tolyl | N-tolyl | b2 | a4 | H |
| 2-20-382 | N-tolyl | N-tolyl | b3 | a4 | H |
| 2-20-383 | N-tolyl | N-tolyl | H | a5 | H |
| 2-20-384 | N-tolyl | N-tolyl | t-bu | a5 | H |
| 2-20-385 | N-tolyl | N-tolyl | methyl | a5 | H |
| 2-20-386 | N-tolyl | N-tolyl | b1 | a5 | H |
| 2-20-387 | N-tolyl | N-tolyl | b2 | a5 | H |
| 2-20-388 | N-tolyl | N-tolyl | b3 | a5 | H |
| 2-20-389 | N-tolyl | N-tolyl | H | a6 | H |
| 2-20-390 | N-tolyl | N-tolyl | t-bu | a6 | H |
| 2-20-391 | N-tolyl | N-tolyl | methyl | a6 | H |
| 2-20-392 | N-tolyl | N-tolyl | b1 | a6 | H |
| 2-20-393 | N-tolyl | N-tolyl | b2 | a6 | H |
| 2-20-394 | N-tolyl | N-tolyl | b3 | a6 | H |
| 2-20-395 | N-tolyl | N-tolyl | H | b1 | H |
| 2-20-396 | N-tolyl | N-tolyl | t-bu | b1 | H |
| 2-20-397 | N-tolyl | N-tolyl | methyl | b1 | H |
| 2-20-398 | N-tolyl | N-tolyl | b1 | b1 | H |
| 2-20-399 | N-tolyl | N-tolyl | b2 | b1 | H |
| 2-20-400 | N-tolyl | N-tolyl | b3 | b1 | H |
| 2-20-401 | N-tolyl | N-tolyl | H | b2 | H |
| 2-20-402 | N-tolyl | N-tolyl | t-bu | b2 | H |
| 2-20-403 | N-tolyl | N-tolyl | methyl | b2 | H |
| 2-20-404 | N-tolyl | N-tolyl | b1 | b2 | H |
| 2-20-405 | N-tolyl | N-tolyl | b2 | b2 | H |
| 2-20-406 | N-tolyl | N-tolyl | b3 | b2 | H |
| 2-20-407 | N-tolyl | N-tolyl | H | b3 | H |
| 2-20-408 | N-tolyl | N-tolyl | t-bu | b3 | H |
| 2-20-409 | N-tolyl | N-tolyl | methyl | b3 | H |
| 2-20-410 | N-tolyl | N-tolyl | b1 | b3 | H |
| 2-20-411 | N-tolyl | N-tolyl | b2 | b3 | H |
| 2-20-412 | N-tolyl | N-tolyl | b3 | b3 | H |
| 2-20-413 | N-tolyl | N-tolyl | H | b4 | H |
| 2-20-414 | N-tolyl | N-tolyl | t-bu | b4 | H |
| 2-20-415 | N-tolyl | N-tolyl | methyl | b4 | H |
| 2-20-416 | N-tolyl | N-tolyl | b1 | b4 | H |
| 2-20-417 | N-tolyl | N-tolyl | b2 | b4 | H |
| 2-20-418 | N-tolyl | N-tolyl | b3 | b4 | H |
| 2-20-419 | N-tolyl | N-tolyl | H | b5 | H |
| 2-20-420 | N-tolyl | N-tolyl | t-bu | b5 | H |
| 2-20-421 | N-tolyl | N-tolyl | methyl | b5 | H |
| 2-20-422 | N-tolyl | N-tolyl | b1 | b5 | H |
| 2-20-423 | N-tolyl | N-tolyl | b2 | b5 | H |
| 2-20-424 | N-tolyl | N-tolyl | b3 | b5 | H |
| 2-20-425 | N-tolyl | N-tolyl | H | b6 | H |
| 2-20-426 | N-tolyl | N-tolyl | t-bu | b6 | H |
| 2-20-427 | N-tolyl | N-tolyl | methyl | b6 | H |
| 2-20-428 | N-tolyl | N-tolyl | b1 | b6 | H |
| 2-20-429 | N-tolyl | N-tolyl | b2 | b6 | H |
| 2-20-430 | N-tolyl | N-tolyl | b3 | b6 | H |
| 2-20-431 | N-tolyl | N-tolyl | H | b7 | H |
| 2-20-432 | N-tolyl | N-tolyl | t-bu | b7 | H |
| 2-20-433 | N-tolyl | N-tolyl | methyl | b7 | H |
| 2-20-434 | N-tolyl | N-tolyl | b1 | b7 | H |
| 2-20-435 | N-tolyl | N-tolyl | b2 | b7 | H |
| 2-20-436 | N-tolyl | N-tolyl | b3 | b7 | H |
| 2-20-437 | N-tolyl | N-tolyl | H | b8 | H |
| 2-20-438 | N-tolyl | N-tolyl | t-bu | b8 | H |
| 2-20-439 | N-tolyl | N-tolyl | methyl | b8 | H |
| 2-20-440 | N-tolyl | N-tolyl | b1 | b8 | H |
| 2-20-441 | N-tolyl | N-tolyl | b2 | b8 | H |
| 2-20-442 | N-tolyl | N-tolyl | b3 | b8 | H |
| 2-20-443 | N-tolyl | N-tolyl | H | b9 | H |
| 2-20-444 | N-tolyl | N-tolyl | t-bu | b9 | H |
| 2-20-445 | N-tolyl | N-tolyl | methyl | b9 | H |
| 2-20-446 | N-tolyl | N-tolyl | b1 | b9 | H |
| 2-20-447 | N-tolyl | N-tolyl | b2 | b9 | H |
| 2-20-448 | N-tolyl | N-tolyl | b3 | b9 | H |
| 2-20-449 | N-tolyl | N-tolyl | H | b10 | H |
| 2-20-450 | N-tolyl | N-tolyl | t-bu | b10 | H |
| 2-20-451 | N-tolyl | N-tolyl | methyl | b10 | H |
| 2-20-452 | N-tolyl | N-tolyl | b1 | b10 | H |
| 2-20-453 | N-tolyl | N-tolyl | b2 | b10 | H |
| 2-20-454 | N-tolyl | N-tolyl | b3 | b10 | H |
| 2-20-455 | N-tolyl | N-tolyl | H | a1 | a1 |
| 2-20-456 | N-tolyl | N-tolyl | t-bu | a1 | a1 |
| 2-20-457 | N-tolyl | N-tolyl | methyl | a1 | a1 |
| 2-20-458 | N-tolyl | N-tolyl | b1 | a1 | a1 |
| 2-20-459 | N-tolyl | N-tolyl | b2 | a1 | a1 |
| 2-20-460 | N-tolyl | N-tolyl | b3 | a1 | a1 |
| 2-20-461 | N-tolyl | N-tolyl | H | a2 | a2 |
| 2-20-462 | N-tolyl | N-tolyl | t-bu | a2 | a2 |
| 2-20-463 | N-tolyl | N-tolyl | methyl | a2 | a2 |
| 2-20-464 | N-tolyl | N-tolyl | b1 | a2 | a2 |
| 2-20-465 | N-tolyl | N-tolyl | b2 | a2 | a2 |
| 2-20-466 | N-tolyl | N-tolyl | b3 | a2 | a2 |
| 2-20-467 | N-tolyl | N-tolyl | H | a3 | a3 |
| 2-20-468 | N-tolyl | N-tolyl | t-bu | a3 | a3 |
| 2-20-469 | N-tolyl | N-tolyl | methyl | a3 | a3 |
| 2-20-470 | N-tolyl | N-tolyl | b1 | a3 | a3 |
| 2-20-471 | N-tolyl | N-tolyl | b2 | a3 | a3 |
| 2-20-472 | N-tolyl | N-tolyl | b3 | a3 | a3 |
| 2-20-473 | N-tolyl | N-tolyl | H | b1 | b1 |
| 2-20-474 | N-tolyl | N-tolyl | t-bu | b1 | b1 |
| 2-20-475 | N-tolyl | N-tolyl | methyl | b1 | b1 |
| 2-20-476 | N-tolyl | N-tolyl | b1 | b1 | b1 |
| 2-20-477 | N-tolyl | N-tolyl | b2 | b1 | b1 |
| 2-20-478 | N-tolyl | N-tolyl | b3 | b1 | b1 |
| 2-20-479 | N-tolyl | N-tolyl | H | b2 | b2 |
| 2-20-480 | N-tolyl | N-tolyl | t-bu | b2 | b2 |
| 2-20-481 | N-tolyl | N-tolyl | methyl | b2 | b2 |
| 2-20-482 | N-tolyl | N-tolyl | b1 | b2 | b2 |
| 2-20-483 | N-tolyl | N-tolyl | b2 | b2 | b2 |
| 2-20-484 | N-tolyl | N-tolyl | b3 | b2 | b2 |
| 2-20-485 | N-tolyl | N-tolyl | H | b3 | b3 |
| 2-20-486 | N-tolyl | N-tolyl | t-bu | b3 | b3 |
| 2-20-487 | N-tolyl | N-tolyl | methyl | b3 | b3 |
| 2-20-488 | N-tolyl | N-tolyl | b1 | b3 | b3 |
| 2-20-489 | N-tolyl | N-tolyl | b2 | b3 | b3 |
| 2-20-490 | N-tolyl | N-tolyl | b3 | b3 | b3 |
| 2-20-491 | N-tolyl | N-tolyl | H | b5 | b5 |
| 2-20-492 | N-tolyl | N-tolyl | t-bu | b5 | b5 |
| 2-20-493 | N-tolyl | N-tolyl | methyl | b5 | b5 |
| 2-20-494 | N-tolyl | N-tolyl | b1 | b5 | b5 |
| 2-20-495 | N-tolyl | N-tolyl | b2 | b5 | b5 |
| 2-20-496 | N-tolyl | N-tolyl | b3 | b5 | b5 |
| 2-20-497 | N-tolyl | N-tolyl | H | b6 | b6 |
| 2-20-498 | N-tolyl | N-tolyl | t-bu | b6 | b6 |
| 2-20-499 | N-tolyl | N-tolyl | methyl | b6 | b6 |
| 2-20-500 | N-tolyl | N-tolyl | b1 | b6 | b6 |
| 2-20-501 | N-tolyl | N-tolyl | b2 | b6 | b6 |
| 2-20-502 | N-tolyl | N-tolyl | b3 | b6 | b6 |
| 2-20-503 | N-tolyl | N-tolyl | H | b7 | b7 |
| 2-20-504 | N-tolyl | N-tolyl | t-bu | b7 | b7 |
| 2-20-505 | N-tolyl | N-tolyl | methyl | b7 | b7 |
| 2-20-506 | N-tolyl | N-tolyl | b1 | b7 | b7 |
| 2-20-507 | N-tolyl | N-tolyl | b2 | b7 | b7 |
| 2-20-508 | N-tolyl | N-tolyl | b3 | b7 | b7 |
| 2-20-509 | N-Biph | N-Biph | H | H | H |
| 2-20-510 | N-Biph | N-Biph | t-bu | H | H |
| 2-20-511 | N-Biph | N-Biph | methyl | H | H |
| 2-20-512 | N-Biph | N-Biph | t-bu | t-bu | t-bu |
| 2-20-513 | N-Biph | N-Biph | methyl | methyl | methyl |
| 2-20-514 | N-Biph | N-Biph | b1 | H | H |
| 2-20-515 | N-Biph | N-Biph | b2 | H | H |
| 2-20-516 | N-Biph | N-Biph | b3 | H | H |
| 2-20-517 | N-Biph | N-Biph | H | H | a1 |
| 2-20-518 | N-Biph | N-Biph | t-bu | H | a1 |
| 2-20-519 | N-Biph | N-Biph | methyl | H | a1 |
| 2-20-520 | N-Biph | N-Biph | b1 | H | a1 |
| 2-20-521 | N-Biph | N-Biph | b2 | H | a1 |
| 2-20-522 | N-Biph | N-Biph | b3 | H | a1 |
| 2-20-523 | N-Biph | N-Biph | H | H | a2 |
| 2-20-524 | N-Biph | N-Biph | t-bu | H | a2 |
| 2-20-525 | N-Biph | N-Biph | methyl | H | a2 |
| 2-20-526 | N-Biph | N-Biph | b1 | H | a2 |
| 2-20-527 | N-Biph | N-Biph | b2 | H | a2 |
| 2-20-528 | N-Biph | N-Biph | b3 | H | a2 |
| 2-20-529 | N-Biph | N-Biph | H | H | a3 |
| 2-20-530 | N-Biph | N-Biph | t-bu | H | a3 |
| 2-20-531 | N-Biph | N-Biph | methyl | H | a3 |
| 2-20-532 | N-Biph | N-Biph | b1 | H | a3 |
| 2-20-533 | N-Biph | N-Biph | b2 | H | a3 |
| 2-20-534 | N-Biph | N-Biph | b3 | H | a3 |
| 2-20-535 | N-Biph | N-Biph | H | H | a4 |
| 2-20-536 | N-Biph | N-Biph | t-bu | H | a4 |
| 2-20-537 | N-Biph | N-Biph | methyl | H | a4 |
| 2-20-538 | N-Biph | N-Biph | b1 | H | a4 |
| 2-20-539 | N-Biph | N-Biph | b2 | H | a4 |
| 2-20-540 | N-Biph | N-Biph | b3 | H | a4 |
| 2-20-541 | N-Biph | N-Biph | H | H | a5 |
| 2-20-542 | N-Biph | N-Biph | t-bu | H | a5 |
| 2-20-543 | N-Biph | N-Biph | methyl | H | a5 |
| 2-20-544 | N-Biph | N-Biph | b1 | H | a5 |
| 2-20-545 | N-Biph | N-Biph | b2 | H | a5 |
| 2-20-546 | N-Biph | N-Biph | b3 | H | a5 |
| 2-20-547 | N-Biph | N-Biph | H | H | a6 |
| 2-20-548 | N-Biph | N-Biph | t-bu | H | a6 |
| 2-20-549 | N-Biph | N-Biph | methyl | H | a6 |
| 2-20-550 | N-Biph | N-Biph | b1 | H | a6 |
| 2-20-551 | N-Biph | N-Biph | b2 | H | a6 |
| 2-20-552 | N-Biph | N-Biph | b3 | H | a6 |
| 2-20-553 | N-Biph | N-Biph | H | H | b1 |
| 2-20-554 | N-Biph | N-Biph | t-bu | H | b1 |
| 2-20-555 | N-Biph | N-Biph | methyl | H | b1 |
| 2-20-556 | N-Biph | N-Biph | b1 | H | b1 |
| 2-20-557 | N-Biph | N-Biph | b2 | H | b1 |
| 2-20-558 | N-Biph | N-Biph | b3 | H | b1 |
| 2-20-559 | N-Biph | N-Biph | H | H | b2 |
| 2-20-560 | N-Biph | N-Biph | t-bu | H | b2 |
| 2-20-561 | N-Biph | N-Biph | methyl | H | b2 |
| 2-20-562 | N-Biph | N-Biph | b1 | H | b2 |
| 2-20-563 | N-Biph | N-Biph | b2 | H | b2 |
| 2-20-564 | N-Biph | N-Biph | b3 | H | b2 |
| 2-20-565 | N-Biph | N-Biph | H | H | b3 |
| 2-20-566 | N-Biph | N-Biph | t-bu | H | b3 |
| 2-20-567 | N-Biph | N-Biph | methyl | H | b3 |
| 2-20-568 | N-Biph | N-Biph | b1 | H | b3 |
| 2-20-569 | N-Biph | N-Biph | b2 | H | b3 |
| 2-20-570 | N-Biph | N-Biph | b3 | H | b3 |
| 2-20-571 | N-Biph | N-Biph | H | H | b4 |
| 2-20-572 | N-Biph | N-Biph | t-bu | H | b4 |
| 2-20-573 | N-Biph | N-Biph | methyl | H | b4 |
| 2-20-574 | N-Biph | N-Biph | b1 | H | b4 |
| 2-20-575 | N-Biph | N-Biph | b2 | H | b4 |
| 2-20-576 | N-Biph | N-Biph | b3 | H | b4 |
| 2-20-577 | N-Biph | N-Biph | H | H | b5 |
| 2-20-578 | N-Biph | N-Biph | t-bu | H | b5 |
| 2-20-579 | N-Biph | N-Biph | methyl | H | b5 |
| 2-20-580 | N-Biph | N-Biph | b1 | H | b5 |
| 2-20-581 | N-Biph | N-Biph | b2 | H | b5 |
| 2-20-582 | N-Biph | N-Biph | b3 | H | b5 |
| 2-20-583 | N-Biph | N-Biph | H | H | b6 |
| 2-20-584 | N-Biph | N-Biph | t-bu | H | b6 |
| 2-20-585 | N-Biph | N-Biph | methyl | H | b6 |
| 2-20-586 | N-Biph | N-Biph | b1 | H | b6 |
| 2-20-587 | N-Biph | N-Biph | b2 | H | b6 |
| 2-20-588 | N-Biph | N-Biph | b3 | H | b6 |
| 2-20-589 | N-Biph | N-Biph | H | H | b7 |
| 2-20-590 | N-Biph | N-Biph | t-bu | H | b7 |
| 2-20-591 | N-Biph | N-Biph | methyl | H | b7 |
| 2-20-592 | N-Biph | N-Biph | b1 | H | b7 |
| 2-20-593 | N-Biph | N-Biph | b2 | H | b7 |
| 2-20-594 | N-Biph | N-Biph | b3 | H | b7 |
| 2-20-595 | N-Biph | N-Biph | H | H | b8 |
| 2-20-596 | N-Biph | N-Biph | t-bu | H | b8 |
| 2-20-597 | N-Biph | N-Biph | methyl | H | b8 |
| 2-20-598 | N-Biph | N-Biph | b1 | H | b8 |
| 2-20-599 | N-Biph | N-Biph | b2 | H | b8 |
| 2-20-600 | N-Biph | N-Biph | b3 | H | b8 |
| 2-20-601 | N-Biph | N-Biph | H | H | b9 |
| 2-20-602 | N-Biph | N-Biph | t-bu | H | b9 |
| 2-20-603 | N-Biph | N-Biph | methyl | H | b9 |
| 2-20-604 | N-Biph | N-Biph | b1 | H | b9 |
| 2-20-605 | N-Biph | N-Biph | b2 | H | b9 |
| 2-20-606 | N-Biph | N-Biph | b3 | H | b9 |
| 2-20-607 | N-Biph | N-Biph | H | H | b10 |
| 2-20-608 | N-Biph | N-Biph | t-bu | H | b10 |
| 2-20-609 | N-Biph | N-Biph | methyl | H | b10 |
| 2-20-610 | N-Biph | N-Biph | b1 | H | b10 |
| 2-20-611 | N-Biph | N-Biph | b2 | H | b10 |
| 2-20-612 | N-Biph | N-Biph | b3 | H | b10 |
| 2-20-613 | N-Biph | N-Biph | H | a1 | H |
| 2-20-614 | N-Biph | N-Biph | t-bu | a1 | H |
| 2-20-615 | N-Biph | N-Biph | methyl | a1 | H |
| 2-20-616 | N-Biph | N-Biph | b1 | a1 | H |
| 2-20-617 | N-Biph | N-Biph | b2 | a1 | H |
| 2-20-618 | N-Biph | N-Biph | b3 | a1 | H |
| 2-20-619 | N-Biph | N-Biph | H | a2 | H |
| 2-20-620 | N-Biph | N-Biph | t-bu | a2 | H |
| 2-20-621 | N-Biph | N-Biph | methyl | a2 | H |
| 2-20-622 | N-Biph | N-Biph | b1 | a2 | H |
| 2-20-623 | N-Biph | N-Biph | b2 | a2 | H |
| 2-20-624 | N-Biph | N-Biph | b3 | a2 | H |
| 2-20-625 | N-Biph | N-Biph | H | a3 | H |
| 2-20-626 | N-Biph | N-Biph | t-bu | a3 | H |
| 2-20-627 | N-Biph | N-Biph | methyl | a3 | H |
| 2-20-628 | N-Biph | N-Biph | b1 | a3 | H |
| 2-20-629 | N-Biph | N-Biph | b2 | a3 | H |
| 2-20-630 | N-Biph | N-Biph | b3 | a3 | H |
| 2-20-631 | N-Biph | N-Biph | H | a4 | H |
| 2-20-632 | N-Biph | N-Biph | t-bu | a4 | H |
| 2-20-633 | N-Biph | N-Biph | methyl | a4 | H |
| 2-20-634 | N-Biph | N-Biph | b1 | a4 | H |
| 2-20-635 | N-Biph | N-Biph | b2 | a4 | H |
| 2-20-636 | N-Biph | N-Biph | b3 | a4 | H |
| 2-20-637 | N-Biph | N-Biph | H | a5 | H |
| 2-20-638 | N-Biph | N-Biph | t-bu | a5 | H |
| 2-20-639 | N-Biph | N-Biph | methyl | a5 | H |
| 2-20-640 | N-Biph | N-Biph | b1 | a5 | H |
| 2-20-641 | N-Biph | N-Biph | b2 | a5 | H |
| 2-20-642 | N-Biph | N-Biph | b3 | a5 | H |
| 2-20-643 | N-Biph | N-Biph | H | a6 | H |
| 2-20-644 | N-Biph | N-Biph | t-bu | a6 | H |
| 2-20-645 | N-Biph | N-Biph | methyl | a6 | H |
| 2-20-646 | N-Biph | N-Biph | b1 | a6 | H |
| 2-20-647 | N-Biph | N-Biph | b2 | a6 | H |
| 2-20-648 | N-Biph | N-Biph | b3 | a6 | H |
| 2-20-649 | N-Biph | N-Biph | H | b1 | H |
| 2-20-650 | N-Biph | N-Biph | t-bu | b1 | H |
| 2-20-651 | N-Biph | N-Biph | methyl | b1 | H |
| 2-20-652 | N-Biph | N-Biph | b1 | b1 | H |
| 2-20-653 | N-Biph | N-Biph | b2 | b1 | H |
| 2-20-654 | N-Biph | N-Biph | b3 | b1 | H |
| 2-20-655 | N-Biph | N-Biph | H | b2 | H |
| 2-20-656 | N-Biph | N-Biph | t-bu | b2 | H |
| 2-20-657 | N-Biph | N-Biph | methyl | b2 | H |
| 2-20-658 | N-Biph | N-Biph | b1 | b2 | H |
| 2-20-659 | N-Biph | N-Biph | b2 | b2 | H |
| 2-20-660 | N-Biph | N-Biph | b3 | b2 | H |
| 2-20-661 | N-Biph | N-Biph | H | b3 | H |
| 2-20-662 | N-Biph | N-Biph | t-bu | b3 | H |
| 2-20-663 | N-Biph | N-Biph | methyl | b3 | H |
| 2-20-664 | N-Biph | N-Biph | b1 | b3 | H |
| 2-20-665 | N-Biph | N-Biph | b2 | b3 | H |
| 2-20-666 | N-Biph | N-Biph | b3 | b3 | H |
| 2-20-667 | N-Biph | N-Biph | H | b4 | H |
| 2-20-668 | N-Biph | N-Biph | t-bu | b4 | H |
| 2-20-669 | N-Biph | N-Biph | methyl | b4 | H |
| 2-20-670 | N-Biph | N-Biph | b1 | b4 | H |
| 2-20-671 | N-Biph | N-Biph | b2 | b4 | H |
| 2-20-672 | N-Biph | N-Biph | b3 | b4 | H |
| 2-20-673 | N-Biph | N-Biph | H | b5 | H |
| 2-20-674 | N-Biph | N-Biph | t-bu | b5 | H |
| 2-20-675 | N-Biph | N-Biph | methyl | b5 | H |
| 2-20-676 | N-Biph | N-Biph | b1 | b5 | H |
| 2-20-677 | N-Biph | N-Biph | b2 | b5 | H |
| 2-20-678 | N-Biph | N-Biph | b3 | b5 | H |
| 2-20-679 | N-Biph | N-Biph | H | b6 | H |
| 2-20-680 | N-Biph | N-Biph | t-bu | b6 | H |
| 2-20-681 | N-Biph | N-Biph | methyl | b6 | H |
| 2-20-682 | N-Biph | N-Biph | b1 | b6 | H |
| 2-20-683 | N-Biph | N-Biph | b2 | b6 | H |
| 2-20-684 | N-Biph | N-Biph | b3 | b6 | H |
| 2-20-685 | N-Biph | N-Biph | H | b7 | H |
| 2-20-686 | N-Biph | N-Biph | t-bu | b7 | H |
| 2-20-687 | N-Biph | N-Biph | methyl | b7 | H |
| 2-20-688 | N-Biph | N-Biph | b1 | b7 | H |
| 2-20-689 | N-Biph | N-Biph | b2 | b7 | H |
| 2-20-690 | N-Biph | N-Biph | b3 | b7 | H |
| 2-20-691 | N-Biph | N-Biph | H | b8 | H |
| 2-20-692 | N-Biph | N-Biph | t-bu | b8 | H |
| 2-20-693 | N-Biph | N-Biph | methyl | b8 | H |
| 2-20-694 | N-Biph | N-Biph | b1 | b8 | H |
| 2-20-695 | N-Biph | N-Biph | b2 | b8 | H |
| 2-20-696 | N-Biph | N-Biph | b3 | b8 | H |
| 2-20-697 | N-Biph | N-Biph | H | b9 | H |
| 2-20-698 | N-Biph | N-Biph | t-bu | b9 | H |
| 2-20-699 | N-Biph | N-Biph | methyl | b9 | H |
| 2-20-700 | N-Biph | N-Biph | b1 | b9 | H |
| 2-20-701 | N-Biph | N-Biph | b2 | b9 | H |
| 2-20-702 | N-Biph | N-Biph | b3 | b9 | H |
| 2-20-703 | N-Biph | N-Biph | H | b10 | H |
| 2-20-704 | N-Biph | N-Biph | t-bu | b10 | H |
| 2-20-705 | N-Biph | N-Biph | methyl | b10 | H |
| 2-20-706 | N-Biph | N-Biph | b1 | b10 | H |
| 2-20-707 | N-Biph | N-Biph | b2 | b10 | H |
| 2-20-708 | N-Biph | N-Biph | b3 | b10 | H |
| 2-20-709 | N-Biph | N-Biph | H | a1 | a1 |
| 2-20-710 | N-Biph | N-Biph | t-bu | a1 | a1 |
| 2-20-711 | N-Biph | N-Biph | methyl | a1 | a1 |
| 2-20-712 | N-Biph | N-Biph | b1 | a1 | a1 |
| 2-20-713 | N-Biph | N-Biph | b2 | a1 | a1 |
| 2-20-714 | N-Biph | N-Biph | b3 | a1 | a1 |
| 2-20-715 | N-Biph | N-Biph | H | a2 | a2 |
| 2-20-716 | N-Biph | N-Biph | t-bu | a2 | a2 |
| 2-20-717 | N-Biph | N-Biph | methyl | a2 | a2 |
| 2-20-718 | N-Biph | N-Biph | b1 | a2 | a2 |
| 2-20-719 | N-Biph | N-Biph | b2 | a2 | a2 |
| 2-20-720 | N-Biph | N-Biph | b3 | a2 | a2 |
| 2-20-721 | N-Biph | N-Biph | H | a3 | a3 |
| 2-20-722 | N-Biph | N-Biph | t-bu | a3 | a3 |
| 2-20-723 | N-Biph | N-Biph | methyl | a3 | a3 |
| 2-20-724 | N-Biph | N-Biph | b1 | a3 | a3 |
| 2-20-725 | N-Biph | N-Biph | b2 | a3 | a3 |
| 2-20-726 | N-Biph | N-Biph | b3 | a3 | a3 |
| 2-20-727 | N-Biph | N-Biph | H | b1 | b1 |
| 2-20-728 | N-Biph | N-Biph | t-bu | b1 | b1 |
| 2-20-729 | N-Biph | N-Biph | methyl | b1 | b1 |
| 2-20-730 | N-Biph | N-Biph | b1 | b1 | b1 |
| 2-20-731 | N-Biph | N-Biph | b2 | b1 | b1 |
| 2-20-732 | N-Biph | N-Biph | b3 | b1 | b1 |
| 2-20-733 | N-Biph | N-Biph | H | b2 | b2 |
| 2-20-734 | N-Biph | N-Biph | t-bu | b2 | b2 |
| 2-20-735 | N-Biph | N-Biph | methyl | b2 | b2 |
| 2-20-736 | N-Biph | N-Biph | b1 | b2 | b2 |
| 2-20-737 | N-Biph | N-Biph | b2 | b2 | b2 |
| 2-20-738 | N-Biph | N-Biph | b3 | b2 | b2 |
| 2-20-739 | N-Biph | N-Biph | H | b3 | b3 |
| 2-20-740 | N-Biph | N-Biph | t-bu | b3 | b3 |
| 2-20-741 | N-Biph | N-Biph | methyl | b3 | b3 |
| 2-20-742 | N-Biph | N-Biph | b1 | b3 | b3 |
| 2-20-743 | N-Biph | N-Biph | b2 | b3 | b3 |
| 2-20-744 | N-Biph | N-Biph | b3 | b3 | b3 |
| 2-20-745 | N-Biph | N-Biph | H | b5 | b5 |
| 2-20-746 | N-Biph | N-Biph | t-bu | b5 | b5 |
| 2-20-747 | N-Biph | N-Biph | methyl | b5 | b5 |
| 2-20-748 | N-Biph | N-Biph | b1 | b5 | b5 |
| 2-20-749 | N-Biph | N-Biph | b2 | b5 | b5 |
| 2-20-750 | N-Biph | N-Biph | b3 | b5 | b5 |
| 2-20-751 | N-Biph | N-Biph | H | b6 | b6 |
| 2-20-752 | N-Biph | N-Biph | t-bu | b6 | b6 |
| 2-20-753 | N-Biph | N-Biph | methyl | b6 | b6 |
| 2-20-754 | N-Biph | N-Biph | b1 | b6 | b6 |
| 2-20-755 | N-Biph | N-Biph | b2 | b6 | b6 |
| 2-20-756 | N-Biph | N-Biph | b3 | b6 | b6 |
| 2-20-757 | N-Biph | N-Biph | H | b7 | b7 |
| 2-20-758 | N-Biph | N-Biph | t-bu | b7 | b7 |
| 2-20-759 | N-Biph | N-Biph | methyl | b7 | b7 |
| 2-20-760 | N-Biph | N-Biph | b1 | b7 | b7 |
| 2-20-761 | N-Biph | N-Biph | b2 | b7 | b7 |
| 2-20-762 | N-Biph | N-Biph | b3 | b7 | b7 |
| 2-20-763 | N-Ph | O | H | H | H |
| 2-20-764 | N-Ph | O | t-bu | H | H |
| 2-20-765 | N-Ph | O | methyl | H | H |
| 2-20-766 | N-Ph | O | t-bu | t-bu | t-bu |
| 2-20-767 | N-Ph | O | methyl | methyl | methyl |
| 2-20-768 | N-Ph | O | b1 | H | H |
| 2-20-769 | N-Ph | O | b2 | H | H |
| 2-20-770 | N-Ph | O | b3 | H | H |
| 2-20-771 | N-Ph | O | H | H | a1 |
| 2-20-772 | N-Ph | O | t-bu | H | a1 |
| 2-20-773 | N-Ph | O | methyl | H | a1 |
| 2-20-774 | N-Ph | O | b1 | H | a1 |
| 2-20-775 | N-Ph | O | b2 | H | a1 |
| 2-20-776 | N-Ph | O | b3 | H | a1 |
| 2-20-777 | N-Ph | O | H | H | a2 |
| 2-20-778 | N-Ph | O | t-bu | H | a2 |
| 2-20-779 | N-Ph | O | methyl | H | a2 |
| 2-20-780 | N-Ph | O | b1 | H | a2 |
| 2-20-781 | N-Ph | O | b2 | H | a2 |
| 2-20-782 | N-Ph | O | b3 | H | a2 |
| 2-20-783 | N-Ph | O | H | H | a3 |
| 2-20-784 | N-Ph | O | t-bu | H | a3 |
| 2-20-785 | N-Ph | O | methyl | H | a3 |
| 2-20-786 | N-Ph | O | b1 | H | a3 |
| 2-20-787 | N-Ph | O | b2 | H | a3 |
| 2-20-788 | N-Ph | O | b3 | H | a3 |
| 2-20-789 | N-Ph | O | H | H | a4 |
| 2-20-790 | N-Ph | O | t-bu | H | a4 |
| 2-20-791 | N-Ph | O | methyl | H | a4 |
| 2-20-792 | N-Ph | O | b1 | H | a4 |
| 2-20-793 | N-Ph | O | b2 | H | a4 |
| 2-20-794 | N-Ph | O | b3 | H | a4 |
| 2-20-795 | N-Ph | O | H | H | a5 |
| 2-20-796 | N-Ph | O | t-bu | H | a5 |
| 2-20-797 | N-Ph | O | methyl | H | a5 |
| 2-20-798 | N-Ph | O | b1 | H | a5 |
| 2-20-799 | N-Ph | O | b2 | H | a5 |
| 2-20-800 | N-Ph | O | b3 | H | a5 |
| 2-20-801 | N-Ph | O | H | H | a6 |
| 2-20-802 | N-Ph | O | t-bu | H | a6 |
| 2-20-803 | N-Ph | O | methyl | H | a6 |
| 2-20-804 | N-Ph | O | b1 | H | a6 |
| 2-20-805 | N-Ph | O | b2 | H | a6 |
| 2-20-806 | N-Ph | O | b3 | H | a6 |
| 2-20-807 | N-Ph | O | H | H | b1 |
| 2-20-808 | N-Ph | O | t-bu | H | b1 |
| 2-20-809 | N-Ph | O | methyl | H | b1 |
| 2-20-810 | N-Ph | O | b1 | H | b1 |
| 2-20-811 | N-Ph | O | b2 | H | b1 |
| 2-20-812 | N-Ph | O | b3 | H | b1 |
| 2-20-813 | N-Ph | O | H | H | b2 |
| 2-20-814 | N-Ph | O | t-bu | H | b2 |
| 2-20-815 | N-Ph | O | methyl | H | b2 |
| 2-20-816 | N-Ph | O | b1 | H | b2 |
| 2-20-817 | N-Ph | O | b2 | H | b2 |
| 2-20-818 | N-Ph | O | b3 | H | b2 |
| 2-20-819 | N-Ph | O | H | H | b3 |
| 2-20-820 | N-Ph | O | t-bu | H | b3 |
| 2-20-821 | N-Ph | O | methyl | H | b3 |
| 2-20-822 | N-Ph | O | b1 | H | b3 |
| 2-20-823 | N-Ph | O | b2 | H | b3 |
| 2-20-824 | N-Ph | O | b3 | H | b3 |
| 2-20-825 | N-Ph | O | H | H | b4 |
| 2-20-826 | N-Ph | O | t-bu | H | b4 |
| 2-20-827 | N-Ph | O | methyl | H | b4 |
| 2-20-828 | N-Ph | O | b1 | H | b4 |
| 2-20-829 | N-Ph | O | b2 | H | b4 |
| 2-20-830 | N-Ph | O | b3 | H | b4 |
| 2-20-831 | N-Ph | O | H | H | b5 |
| 2-20-832 | N-Ph | O | t-bu | H | b5 |
| 2-20-833 | N-Ph | O | methyl | H | b5 |
| 2-20-834 | N-Ph | O | b1 | H | b5 |
| 2-20-835 | N-Ph | O | b2 | H | b5 |
| 2-20-836 | N-Ph | O | b3 | H | b5 |
| 2-20-837 | N-Ph | O | H | H | b6 |
| 2-20-838 | N-Ph | O | t-bu | H | b6 |
| 2-20-839 | N-Ph | O | methyl | H | b6 |
| 2-20-840 | N-Ph | O | b1 | H | b6 |
| 2-20-841 | N-Ph | O | b2 | H | b6 |
| 2-20-842 | N-Ph | O | b3 | H | b6 |
| 2-20-843 | N-Ph | O | H | H | b7 |
| 2-20-844 | N-Ph | O | t-bu | H | b7 |
| 2-20-845 | N-Ph | O | methyl | H | b7 |
| 2-20-846 | N-Ph | O | b1 | H | b7 |
| 2-20-847 | N-Ph | O | b2 | H | b7 |
| 2-20-848 | N-Ph | O | b3 | H | b7 |
| 2-20-849 | N-Ph | O | H | H | b8 |
| 2-20-850 | N-Ph | O | t-bu | H | b8 |
| 2-20-851 | N-Ph | O | methyl | H | b8 |
| 2-20-852 | N-Ph | O | b1 | H | b8 |
| 2-20-853 | N-Ph | O | b2 | H | b8 |
| 2-20-854 | N-Ph | O | b3 | H | b8 |
| 2-20-855 | N-Ph | O | H | H | b9 |
| 2-20-856 | N-Ph | O | t-bu | H | b9 |
| 2-20-857 | N-Ph | O | methyl | H | b9 |
| 2-20-858 | N-Ph | O | b1 | H | b9 |
| 2-20-859 | N-Ph | O | b2 | H | b9 |
| 2-20-860 | N-Ph | O | b3 | H | b9 |
| 2-20-861 | N-Ph | O | H | H | b10 |
| 2-20-862 | N-Ph | O | t-bu | H | b10 |
| 2-20-863 | N-Ph | O | methyl | H | b10 |
| 2-20-864 | N-Ph | O | b1 | H | b10 |
| 2-20-865 | N-Ph | O | b2 | H | b10 |
| 2-20-866 | N-Ph | O | b3 | H | b10 |
| 2-20-867 | N-Ph | O | H | a1 | H |
| 2-20-868 | N-Ph | O | t-bu | a1 | H |
| 2-20-869 | N-Ph | O | methyl | a1 | H |
| 2-20-870 | N-Ph | O | b1 | a1 | H |
| 2-20-871 | N-Ph | O | b2 | a1 | H |
| 2-20-872 | N-Ph | O | b3 | a1 | H |
| 2-20-873 | N-Ph | O | H | a2 | H |
| 2-20-874 | N-Ph | O | t-bu | a2 | H |
| 2-20-875 | N-Ph | O | methyl | a2 | H |
| 2-20-876 | N-Ph | O | b1 | a2 | H |
| 2-20-877 | N-Ph | O | b2 | a2 | H |
| 2-20-878 | N-Ph | O | b3 | a2 | H |
| 2-20-879 | N-Ph | O | H | a3 | H |
| 2-20-880 | N-Ph | O | t-bu | a3 | H |
| 2-20-881 | N-Ph | O | methyl | a3 | H |
| 2-20-882 | N-Ph | O | b1 | a3 | H |
| 2-20-883 | N-Ph | O | b2 | a3 | H |
| 2-20-884 | N-Ph | O | b3 | a3 | H |
| 2-20-885 | N-Ph | O | H | a4 | H |
| 2-20-886 | N-Ph | O | t-bu | a4 | H |
| 2-20-887 | N-Ph | O | methyl | a4 | H |
| 2-20-888 | N-Ph | O | b1 | a4 | H |
| 2-20-889 | N-Ph | O | b2 | a4 | H |
| 2-20-890 | N-Ph | O | b3 | a4 | H |
| 2-20-891 | N-Ph | O | H | a5 | H |
| 2-20-892 | N-Ph | O | t-bu | a5 | H |
| 2-20-893 | N-Ph | O | methyl | a5 | H |
| 2-20-894 | N-Ph | O | b1 | a5 | H |
| 2-20-895 | N-Ph | O | b2 | a5 | H |
| 2-20-896 | N-Ph | O | b3 | a5 | H |
| 2-20-897 | N-Ph | O | H | a6 | H |
| 2-20-898 | N-Ph | O | t-bu | a6 | H |
| 2-20-899 | N-Ph | O | methyl | a6 | H |
| 2-20-900 | N-Ph | O | b1 | a6 | H |
| 2-20-901 | N-Ph | O | b2 | a6 | H |
| 2-20-902 | N-Ph | O | b3 | a6 | H |
| 2-20-903 | N-Ph | O | H | b1 | H |
| 2-20-904 | N-Ph | O | t-bu | b1 | H |
| 2-20-905 | N-Ph | O | methyl | b1 | H |
| 2-20-906 | N-Ph | O | b1 | b1 | H |
| 2-20-907 | N-Ph | O | b2 | b1 | H |
| 2-20-908 | N-Ph | O | b3 | b1 | H |
| 2-20-909 | N-Ph | O | H | b2 | H |
| 2-20-910 | N-Ph | O | t-bu | b2 | H |
| 2-20-911 | N-Ph | O | methyl | b2 | H |
| 2-20-912 | N-Ph | O | b1 | b2 | H |
| 2-20-913 | N-Ph | O | b2 | b2 | H |
| 2-20-914 | N-Ph | O | b3 | b2 | H |
| 2-20-915 | N-Ph | O | H | b3 | H |
| 2-20-916 | N-Ph | O | t-bu | b3 | H |
| 2-20-917 | N-Ph | O | methyl | b3 | H |
| 2-20-918 | N-Ph | O | b1 | b3 | H |
| 2-20-919 | N-Ph | O | b2 | b3 | H |
| 2-20-920 | N-Ph | O | b3 | b3 | H |
| 2-20-921 | N-Ph | O | H | b4 | H |
| 2-20-922 | N-Ph | O | t-bu | b4 | H |
| 2-20-923 | N-Ph | O | methyl | b4 | H |
| 2-20-924 | N-Ph | O | b1 | b4 | H |
| 2-20-925 | N-Ph | O | b2 | b4 | H |
| 2-20-926 | N-Ph | O | b3 | b4 | H |
| 2-20-927 | N-Ph | O | H | b5 | H |
| 2-20-928 | N-Ph | O | t-bu | b5 | H |
| 2-20-929 | N-Ph | O | methyl | b5 | H |
| 2-20-930 | N-Ph | O | b1 | b5 | H |
| 2-20-931 | N-Ph | O | b2 | b5 | H |
| 2-20-932 | N-Ph | O | b3 | b5 | H |
| 2-20-933 | N-Ph | O | H | b6 | H |
| 2-20-934 | N-Ph | O | t-bu | b6 | H |
| 2-20-935 | N-Ph | O | methyl | b6 | H |
| 2-20-936 | N-Ph | O | b1 | b6 | H |
| 2-20-937 | N-Ph | O | b2 | b6 | H |
| 2-20-938 | N-Ph | O | b3 | b6 | H |
| 2-20-939 | N-Ph | O | H | b7 | H |
| 2-20-940 | N-Ph | O | t-bu | b7 | H |
| 2-20-941 | N-Ph | O | methyl | b7 | H |
| 2-20-942 | N-Ph | O | b1 | b7 | H |
| 2-20-943 | N-Ph | O | b2 | b7 | H |
| 2-20-944 | N-Ph | O | b3 | b7 | H |
| 2-20-945 | N-Ph | O | H | b8 | H |
| 2-20-946 | N-Ph | O | t-bu | b8 | H |
| 2-20-947 | N-Ph | O | methyl | b8 | H |
| 2-20-948 | N-Ph | O | b1 | b8 | H |
| 2-20-949 | N-Ph | O | b2 | b8 | H |
| 2-20-950 | N-Ph | O | b3 | b8 | H |
| 2-20-951 | N-Ph | O | H | b9 | H |
| 2-20-952 | N-Ph | O | t-bu | b9 | H |
| 2-20-953 | N-Ph | O | methyl | b9 | H |
| 2-20-954 | N-Ph | O | b1 | b9 | H |
| 2-20-955 | N-Ph | O | b2 | b9 | H |
| 2-20-956 | N-Ph | O | b3 | b9 | H |
| 2-20-957 | N-Ph | O | H | b10 | H |
| 2-20-958 | N-Ph | O | t-bu | b10 | H |
| 2-20-959 | N-Ph | O | methyl | b10 | H |
| 2-20-960 | N-Ph | O | b1 | b10 | H |
| 2-20-961 | N-Ph | O | b2 | b10 | H |
| 2-20-962 | N-Ph | O | b3 | b10 | H |
| 2-20-963 | N-Ph | O | H | a1 | a1 |
| 2-20-964 | N-Ph | O | t-bu | a1 | a1 |
| 2-20-965 | N-Ph | O | methyl | a1 | a1 |
| 2-20-966 | N-Ph | O | b1 | a1 | a1 |
| 2-20-967 | N-Ph | O | b2 | a1 | a1 |
| 2-20-968 | N-Ph | O | b3 | a1 | a1 |
| 2-20-969 | N-Ph | O | H | a2 | a2 |
| 2-20-970 | N-Ph | O | t-bu | a2 | a2 |
| 2-20-971 | N-Ph | O | methyl | a2 | a2 |
| 2-20-972 | N-Ph | O | b1 | a2 | a2 |
| 2-20-973 | N-Ph | O | b2 | a2 | a2 |
| 2-20-974 | N-Ph | O | b3 | a2 | a2 |
| 2-20-975 | N-Ph | O | H | a3 | a3 |
| 2-20-976 | N-Ph | O | t-bu | a3 | a3 |
| 2-20-977 | N-Ph | O | methyl | a3 | a3 |
| 2-20-978 | N-Ph | O | b1 | a3 | a3 |
| 2-20-979 | N-Ph | O | b2 | a3 | a3 |
| 2-20-980 | N-Ph | O | b3 | a3 | a3 |
| 2-20-981 | N-Ph | O | H | b1 | b1 |
| 2-20-982 | N-Ph | O | t-bu | b1 | b1 |
| 2-20-983 | N-Ph | O | methyl | b1 | b1 |
| 2-20-984 | N-Ph | O | b1 | b1 | b1 |
| 2-20-985 | N-Ph | O | b2 | b1 | b1 |
| 2-20-986 | N-Ph | O | b3 | b1 | b1 |
| 2-20-987 | N-Ph | O | H | b2 | b2 |
| 2-20-988 | N-Ph | O | t-bu | b2 | b2 |
| 2-20-989 | N-Ph | O | methyl | b2 | b2 |
| 2-20-990 | N-Ph | O | b1 | b2 | b2 |
| 2-20-991 | N-Ph | O | b2 | b2 | b2 |
| 2-20-992 | N-Ph | O | b3 | b2 | b2 |
| 2-20-993 | N-Ph | O | H | b3 | b3 |
| 2-20-994 | N-Ph | O | t-bu | b3 | b3 |
| 2-20-995 | N-Ph | O | methyl | b3 | b3 |
| 2-20-996 | N-Ph | O | b1 | b3 | b3 |
| 2-20-997 | N-Ph | O | b2 | b3 | b3 |
| 2-20-998 | N-Ph | O | b3 | b3 | b3 |
| 2-20-999 | N-Ph | O | H | b5 | b5 |
| 2-20-1000 | N-Ph | O | t-bu | b5 | b5 |
| 2-20-1001 | N-Ph | O | methyl | b5 | b5 |
| 2-20-1002 | N-Ph | O | b1 | b5 | b5 |
| 2-20-1003 | N-Ph | O | b2 | b5 | b5 |
| 2-20-1004 | N-Ph | O | b3 | b5 | b5 |
| 2-20-1005 | N-Ph | O | H | b6 | b6 |
| 2-20-1006 | N-Ph | O | t-bu | b6 | b6 |
| 2-20-1007 | N-Ph | O | methyl | b6 | b6 |
| 2-20-1008 | N-Ph | O | b1 | b6 | b6 |
| 2-20-1009 | N-Ph | O | b2 | b6 | b6 |
| 2-20-1010 | N-Ph | O | b3 | b6 | b6 |
| 2-20-1011 | N-Ph | O | H | b7 | b7 |
| 2-20-1012 | N-Ph | O | t-bu | b7 | b7 |
| 2-20-1013 | N-Ph | O | methyl | b7 | b7 |
| 2-20-1014 | N-Ph | O | b1 | b7 | b7 |
| 2-20-1015 | N-Ph | O | b2 | b7 | b7 |
| 2-20-1016 | N-Ph | O | b3 | b7 | b7 |
| 2-20-1017 | N-Ph | S | H | H | H |
| 2-20-1018 | N-Ph | S | t-bu | H | H |
| 2-20-1019 | N-Ph | S | methyl | H | H |
| 2-20-1020 | N-Ph | S | t-bu | t-bu | t-bu |
| 2-20-1021 | N-Ph | S | methyl | methyl | methyl |
| 2-20-1022 | N-Ph | S | b1 | H | H |
| 2-20-1023 | N-Ph | S | b2 | H | H |
| 2-20-1024 | N-Ph | S | b3 | H | H |
| 2-20-1025 | N-Ph | S | H | H | a1 |
| 2-20-1026 | N-Ph | S | t-bu | H | a1 |
| 2-20-1027 | N-Ph | S | methyl | H | a1 |
| 2-20-1028 | N-Ph | S | b1 | H | a1 |
| 2-20-1029 | N-Ph | S | b2 | H | a1 |
| 2-20-1030 | N-Ph | S | b3 | H | a1 |
| 2-20-1031 | N-Ph | S | H | H | a2 |
| 2-20-1032 | N-Ph | S | t-bu | H | a2 |
| 2-20-1033 | N-Ph | S | methyl | H | a2 |
| 2-20-1034 | N-Ph | S | b1 | H | a2 |
| 2-20-1035 | N-Ph | S | b2 | H | a2 |
| 2-20-1036 | N-Ph | S | b3 | H | a2 |
| 2-20-1037 | N-Ph | S | H | H | a3 |
| 2-20-1038 | N-Ph | S | t-bu | H | a3 |
| 2-20-1039 | N-Ph | S | methyl | H | a3 |
| 2-20-1040 | N-Ph | S | b1 | H | a3 |
| 2-20-1041 | N-Ph | S | b2 | H | a3 |
| 2-20-1042 | N-Ph | S | b3 | H | a3 |
| 2-20-1043 | N-Ph | S | H | H | a4 |
| 2-20-1044 | N-Ph | S | t-bu | H | a4 |
| 2-20-1045 | N-Ph | S | methyl | H | a4 |
| 2-20-1046 | N-Ph | S | b1 | H | a4 |
| 2-20-1047 | N-Ph | S | b2 | H | a4 |
| 2-20-1048 | N-Ph | S | b3 | H | a4 |
| 2-20-1049 | N-Ph | S | H | H | a5 |
| 2-20-1050 | N-Ph | S | t-bu | H | a5 |
| 2-20-1051 | N-Ph | S | methyl | H | a5 |
| 2-20-1052 | N-Ph | S | b1 | H | a5 |
| 2-20-1053 | N-Ph | S | b2 | H | a5 |
| 2-20-1054 | N-Ph | S | b3 | H | a5 |
| 2-20-1055 | N-Ph | S | H | H | a6 |
| 2-20-1056 | N-Ph | S | t-bu | H | a6 |
| 2-20-1057 | N-Ph | S | methyl | H | a6 |
| 2-20-1058 | N-Ph | S | b1 | H | a6 |
| 2-20-1059 | N-Ph | S | b2 | H | a6 |
| 2-20-1060 | N-Ph | S | b3 | H | a6 |
| 2-20-1061 | N-Ph | S | H | H | b1 |
| 2-20-1062 | N-Ph | S | t-bu | H | b1 |
| 2-20-1063 | N-Ph | S | methyl | H | b1 |
| 2-20-1064 | N-Ph | S | b1 | H | b1 |
| 2-20-1065 | N-Ph | S | b2 | H | b1 |
| 2-20-1066 | N-Ph | S | b3 | H | b1 |
| 2-20-1067 | N-Ph | S | H | H | b2 |
| 2-20-1068 | N-Ph | S | t-bu | H | b2 |
| 2-20-1069 | N-Ph | S | methyl | H | b2 |
| 2-20-1070 | N-Ph | S | b1 | H | b2 |
| 2-20-1071 | N-Ph | S | b2 | H | b2 |
| 2-20-1072 | N-Ph | S | b3 | H | b2 |
| 2-20-1073 | N-Ph | S | H | H | b3 |
| 2-20-1074 | N-Ph | S | t-bu | H | b3 |
| 2-20-1075 | N-Ph | S | methyl | H | b3 |
| 2-20-1076 | N-Ph | S | b1 | H | b3 |
| 2-20-1077 | N-Ph | S | b2 | H | b3 |
| 2-20-1078 | N-Ph | S | b3 | H | b3 |
| 2-20-1079 | N-Ph | S | H | H | b4 |
| 2-20-1080 | N-Ph | S | t-bu | H | b4 |
| 2-20-1081 | N-Ph | S | methyl | H | b4 |
| 2-20-1082 | N-Ph | S | b1 | H | b4 |
| 2-20-1083 | N-Ph | S | b2 | H | b4 |
| 2-20-1084 | N-Ph | S | b3 | H | b4 |
| 2-20-1085 | N-Ph | S | H | H | b5 |
| 2-20-1086 | N-Ph | S | t-bu | H | b5 |
| 2-20-1087 | N-Ph | S | methyl | H | b5 |
| 2-20-1088 | N-Ph | S | b1 | H | b5 |
| 2-20-1089 | N-Ph | S | b2 | H | b5 |
| 2-20-1090 | N-Ph | S | b3 | H | b5 |
| 2-20-1091 | N-Ph | S | H | H | b6 |
| 2-20-1092 | N-Ph | S | t-bu | H | b6 |
| 2-20-1093 | N-Ph | S | methyl | H | b6 |
| 2-20-1094 | N-Ph | S | b1 | H | b6 |
| 2-20-1095 | N-Ph | S | b2 | H | b6 |
| 2-20-1096 | N-Ph | S | b3 | H | b6 |
| 2-20-1097 | N-Ph | S | H | H | b7 |
| 2-20-1098 | N-Ph | S | t-bu | H | b7 |
| 2-20-1099 | N-Ph | S | methyl | H | b7 |
| 2-20-1100 | N-Ph | S | b1 | H | b7 |
| 2-20-1101 | N-Ph | S | b2 | H | b7 |
| 2-20-1102 | N-Ph | S | b3 | H | b7 |
| 2-20-1103 | N-Ph | S | H | H | b8 |
| 2-20-1104 | N-Ph | S | t-bu | H | b8 |
| 2-20-1105 | N-Ph | S | methyl | H | b8 |
| 2-20-1106 | N-Ph | S | b1 | H | b8 |
| 2-20-1107 | N-Ph | S | b2 | H | b8 |
| 2-20-1108 | N-Ph | S | b3 | H | b8 |
| 2-20-1109 | N-Ph | S | H | H | b9 |
| 2-20-1110 | N-Ph | S | t-bu | H | b9 |
| 2-20-1111 | N-Ph | S | methyl | H | b9 |
| 2-20-1112 | N-Ph | S | b1 | H | b9 |
| 2-20-1113 | N-Ph | S | b2 | H | b9 |
| 2-20-1114 | N-Ph | S | b3 | H | b9 |
| 2-20-1115 | N-Ph | S | H | H | b10 |
| 2-20-1116 | N-Ph | S | t-bu | H | b10 |
| 2-20-1117 | N-Ph | S | methyl | H | b10 |
| 2-20-1118 | N-Ph | S | b1 | H | b10 |
| 2-20-1119 | N-Ph | S | b2 | H | b10 |
| 2-20-1120 | N-Ph | S | b3 | H | b10 |
| 2-20-1121 | N-Ph | S | H | a1 | H |
| 2-20-1122 | N-Ph | S | t-bu | a1 | H |
| 2-20-1123 | N-Ph | S | methyl | a1 | H |
| 2-20-1124 | N-Ph | S | b1 | a1 | H |
| 2-20-1125 | N-Ph | S | b2 | a1 | H |
| 2-20-1126 | N-Ph | S | b3 | a1 | H |
| 2-20-1127 | N-Ph | S | H | a2 | H |
| 2-20-1128 | N-Ph | S | t-bu | a2 | H |
| 2-20-1129 | N-Ph | S | methyl | a2 | H |
| 2-20-1130 | N-Ph | S | b1 | a2 | H |
| 2-20-1131 | N-Ph | S | b2 | a2 | H |
| 2-20-1132 | N-Ph | S | b3 | a2 | H |
| 2-20-1133 | N-Ph | S | H | a3 | H |
| 2-20-1134 | N-Ph | S | t-bu | a3 | H |
| 2-20-1135 | N-Ph | S | methyl | a3 | H |
| 2-20-1136 | N-Ph | S | b1 | a3 | H |
| 2-20-1137 | N-Ph | S | b2 | a3 | H |
| 2-20-1138 | N-Ph | S | b3 | a3 | H |
| 2-20-1139 | N-Ph | S | H | a4 | H |
| 2-20-1140 | N-Ph | S | t-bu | a4 | H |
| 2-20-1141 | N-Ph | S | methyl | a4 | H |
| 2-20-1142 | N-Ph | S | b1 | a4 | H |
| 2-20-1143 | N-Ph | S | b2 | a4 | H |
| 2-20-1144 | N-Ph | S | b3 | a4 | H |
| 2-20-1145 | N-Ph | S | H | a5 | H |
| 2-20-1146 | N-Ph | S | t-bu | a5 | H |
| 2-20-1147 | N-Ph | S | methyl | a5 | H |
| 2-20-1148 | N-Ph | S | b1 | a5 | H |
| 2-20-1149 | N-Ph | S | b2 | a5 | H |
| 2-20-1150 | N-Ph | S | b3 | a5 | H |
| 2-20-1151 | N-Ph | S | H | a6 | H |
| 2-20-1152 | N-Ph | S | t-bu | a6 | H |
| 2-20-1153 | N-Ph | S | methyl | a6 | H |
| 2-20-1154 | N-Ph | S | b1 | a6 | H |
| 2-20-1155 | N-Ph | S | b2 | a6 | H |
| 2-20-1156 | N-Ph | S | b3 | a6 | H |
| 2-20-1157 | N-Ph | S | H | b1 | H |
| 2-20-1158 | N-Ph | S | t-bu | b1 | H |
| 2-20-1159 | N-Ph | S | methyl | b1 | H |
| 2-20-1160 | N-Ph | S | b1 | b1 | H |
| 2-20-1161 | N-Ph | S | b2 | b1 | H |
| 2-20-1162 | N-Ph | S | b3 | b1 | H |
| 2-20-1163 | N-Ph | S | H | b2 | H |
| 2-20-1164 | N-Ph | S | t-bu | b2 | H |
| 2-20-1165 | N-Ph | S | methyl | b2 | H |
| 2-20-1166 | N-Ph | S | b1 | b2 | H |
| 2-20-1167 | N-Ph | S | b2 | b2 | H |
| 2-20-1168 | N-Ph | S | b3 | b2 | H |
| 2-20-1169 | N-Ph | S | H | b3 | H |
| 2-20-1170 | N-Ph | S | t-bu | b3 | H |
| 2-20-1171 | N-Ph | S | methyl | b3 | H |
| 2-20-1172 | N-Ph | S | b1 | b3 | H |
| 2-20-1173 | N-Ph | S | b2 | b3 | H |
| 2-20-1174 | N-Ph | S | b3 | b3 | H |
| 2-20-1175 | N-Ph | S | H | b4 | H |
| 2-20-1176 | N-Ph | S | t-bu | b4 | H |
| 2-20-1177 | N-Ph | S | methyl | b4 | H |
| 2-20-1178 | N-Ph | S | b1 | b4 | H |
| 2-20-1179 | N-Ph | S | b2 | b4 | H |
| 2-20-1180 | N-Ph | S | b3 | b4 | H |
| 2-20-1181 | N-Ph | S | H | b5 | H |
| 2-20-1182 | N-Ph | S | t-bu | b5 | H |
| 2-20-1183 | N-Ph | S | methyl | b5 | H |
| 2-20-1184 | N-Ph | S | b1 | b5 | H |
| 2-20-1185 | N-Ph | S | b2 | b5 | H |
| 2-20-1186 | N-Ph | S | b3 | b5 | H |
| 2-20-1187 | N-Ph | S | H | b6 | H |
| 2-20-1188 | N-Ph | S | t-bu | b6 | H |
| 2-20-1189 | N-Ph | S | methyl | b6 | H |
| 2-20-1190 | N-Ph | S | b1 | b6 | H |
| 2-20-1191 | N-Ph | S | b2 | b6 | H |
| 2-20-1192 | N-Ph | S | b3 | b6 | H |
| 2-20-1193 | N-Ph | S | H | b7 | H |
| 2-20-1194 | N-Ph | S | t-bu | b7 | H |
| 2-20-1195 | N-Ph | S | methyl | b7 | H |
| 2-20-1196 | N-Ph | S | b1 | b7 | H |
| 2-20-1197 | N-Ph | S | b2 | b7 | H |
| 2-20-1198 | N-Ph | S | b3 | b7 | H |
| 2-20-1199 | N-Ph | S | H | b8 | H |
| 2-20-1200 | N-Ph | S | t-bu | b8 | H |
| 2-20-1201 | N-Ph | S | methyl | b8 | H |
| 2-20-1202 | N-Ph | S | b1 | b8 | H |
| 2-20-1203 | N-Ph | S | b2 | b8 | H |
| 2-20-1204 | N-Ph | S | b3 | b8 | H |
| 2-20-1205 | N-Ph | S | H | b9 | H |
| 2-20-1206 | N-Ph | S | t-bu | b9 | H |
| 2-20-1207 | N-Ph | S | methyl | b9 | H |
| 2-20-1208 | N-Ph | S | b1 | b9 | H |
| 2-20-1209 | N-Ph | S | b2 | b9 | H |
| 2-20-1210 | N-Ph | S | b3 | b9 | H |
| 2-20-1211 | N-Ph | S | H | b10 | H |
| 2-20-1212 | N-Ph | S | t-bu | b10 | H |
| 2-20-1213 | N-Ph | S | methyl | b10 | H |
| 2-20-1214 | N-Ph | S | b1 | b10 | H |
| 2-20-1215 | N-Ph | S | b2 | b10 | H |
| 2-20-1216 | N-Ph | S | b3 | b10 | H |
| 2-20-1217 | N-Ph | S | H | a1 | a1 |
| 2-20-1218 | N-Ph | S | t-bu | a1 | a1 |
| 2-20-1219 | N-Ph | S | methyl | a1 | a1 |
| 2-20-1220 | N-Ph | S | b1 | a1 | a1 |
| 2-20-1221 | N-Ph | S | b2 | a1 | a1 |
| 2-20-1222 | N-Ph | S | b3 | a1 | a1 |
| 2-20-1223 | N-Ph | S | H | a2 | a2 |
| 2-20-1224 | N-Ph | S | t-bu | a2 | a2 |
| 2-20-1225 | N-Ph | S | methyl | a2 | a2 |
| 2-20-1226 | N-Ph | S | b1 | a2 | a2 |
| 2-20-1227 | N-Ph | S | b2 | a2 | a2 |
| 2-20-1228 | N-Ph | S | b3 | a2 | a2 |
| 2-20-1229 | N-Ph | S | H | a3 | a3 |
| 2-20-1230 | N-Ph | S | t-bu | a3 | a3 |
| 2-20-1231 | N-Ph | S | methyl | a3 | a3 |
| 2-20-1232 | N-Ph | S | b1 | a3 | a3 |
| 2-20-1233 | N-Ph | S | b2 | a3 | a3 |
| 2-20-1234 | N-Ph | S | b3 | a3 | a3 |
| 2-20-1235 | N-Ph | S | H | b1 | b1 |
| 2-20-1236 | N-Ph | S | t-bu | b1 | b1 |
| 2-20-1237 | N-Ph | S | methyl | b1 | b1 |
| 2-20-1238 | N-Ph | S | b1 | b1 | b1 |
| 2-20-1239 | N-Ph | S | b2 | b1 | b1 |
| 2-20-1240 | N-Ph | S | b3 | b1 | b1 |
| 2-20-1241 | N-Ph | S | H | b2 | b2 |
| 2-20-1242 | N-Ph | S | t-bu | b2 | b2 |
| 2-20-1243 | N-Ph | S | methyl | b2 | b2 |
| 2-20-1244 | N-Ph | S | b1 | b2 | b2 |
| 2-20-1245 | N-Ph | S | b2 | b2 | b2 |
| 2-20-1246 | N-Ph | S | b3 | b2 | b2 |
| 2-20-1247 | N-Ph | S | H | b3 | b3 |
| 2-20-1248 | N-Ph | S | t-bu | b3 | b3 |
| 2-20-1249 | N-Ph | S | methyl | b3 | b3 |
| 2-20-1250 | N-Ph | S | b1 | b3 | b3 |
| 2-20-1251 | N-Ph | S | b2 | b3 | b3 |
| 2-20-1252 | N-Ph | S | b3 | b3 | b3 |
| 2-20-1253 | N-Ph | S | H | b5 | b5 |
| 2-20-1254 | N-Ph | S | t-bu | b5 | b5 |
| 2-20-1255 | N-Ph | S | methyl | b5 | b5 |
| 2-20-1256 | N-Ph | S | b1 | b5 | b5 |
| 2-20-1257 | N-Ph | S | b2 | b5 | b5 |
| 2-20-1258 | N-Ph | S | b3 | b5 | b5 |
| 2-20-1259 | N-Ph | S | H | b6 | b6 |
| 2-20-1260 | N-Ph | S | t-bu | b6 | b6 |
| 2-20-1261 | N-Ph | S | methyl | b6 | b6 |
| 2-20-1262 | N-Ph | S | b1 | b6 | b6 |
| 2-20-1263 | N-Ph | S | b2 | b6 | b6 |
| 2-20-1264 | N-Ph | S | b3 | b6 | b6 |
| 2-20-1265 | N-Ph | S | H | b7 | b7 |
| 2-20-1266 | N-Ph | 5 | t-bu | b7 | b7 |
| 2-20-1267 | N-Ph | S | methyl | b7 | b7 |
| 2-20-1268 | N-Ph | S | b1 | b7 | b7 |
| 2-20-1269 | N-Ph | S | b2 | b7 | b7 |
| 2-20-1270 | N-Ph | S | b3 | b7 | b7 |

Group 2-21
The compounds are represented by the formula 2-21 below, wherein CY₃ is a benzene group, X₁ is B, and Y1, Y2, Y3, R1b, R2b, (R3)a3 are each shown in the table 21 below, 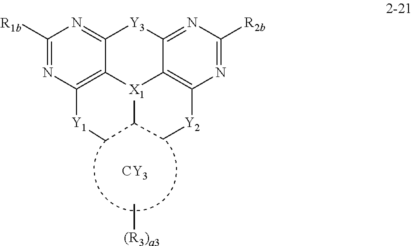
TABLE 21

| Compound | | | | | |
|---|---|---|---|---|---|
| No. | Y1/Y2 | Y3 | R1b | R2b | (R3)a3 |
| 2-21-001 | N—Ph | N—Ph | H | H | H |
| 2-21-002 | N—Ph | N—Ph | t-bu | t-bu | H |
| 2-21-003 | N—Ph | N—Ph | methyl | methyl | H |
| 2-21-004 | N—Ph | N—Ph | t-bu | t-bu | t-bu |
| 2-21-005 | N—Ph | N—Ph | methyl | methyl | methyl |
| 2-21-006 | N—Ph | N—Ph | b1 | b1 | H |
| 2-21-007 | N—Ph | N—Ph | b2 | b2 | H |
| 2-21-008 | N—Ph | N—Ph | b3 | b3 | H |
| 2-21-009 | N—Ph | N—Ph | H | H | a1 |
| 2-21-010 | N—Ph | N—Ph | t-bu | t-bu | a1 |
| 2-21-011 | N—Ph | N—Ph | methyl | methyl | a1 |
| 2-21-012 | N—Ph | N—Ph | b1 | b1 | a1 |
| 2-21-013 | N—Ph | N—Ph | b2 | b2 | a1 |
| 2-21-014 | N—Ph | N—Ph | b3 | b3 | a1 |
| 2-21-015 | N—Ph | N—Ph | H | H | a2 |
| 2-21-016 | N—Ph | N—Ph | t-bu | t-bu | a2 |
| 2-21-017 | N—Ph | N—Ph | methyl | methyl | a2 |
| 2-21-018 | N—Ph | N—Ph | b1 | b1 | a2 |
| 2-21-019 | N—Ph | N—Ph | b2 | b2 | a2 |
| 2-21-020 | N—Ph | N—Ph | b3 | b3 | a2 |
| 2-21-021 | N—Ph | N—Ph | H | H | a3 |
| 2-21-022 | N—Ph | N—Ph | t-bu | t-bu | a3 |
| 2-21-023 | N—Ph | N—Ph | methyl | methyl | a3 |
| 2-21-024 | N—Ph | N—Ph | b1 | b1 | a3 |
| 2-21-025 | N—Ph | N—Ph | b2 | b2 | a3 |
| 2-21-026 | N—Ph | N—Ph | b3 | b3 | a3 |
| 2-21-027 | N—Ph | N—Ph | H | H | a4 |
| 2-21-028 | N—Ph | N—Ph | t-bu | t-bu | a4 |
| 2-21-029 | N—Ph | N—Ph | methyl | methyl | a4 |
| 2-21-030 | N—Ph | N—Ph | b1 | b1 | a4 |
| 2-21-031 | N—Ph | N—Ph | b2 | b2 | a4 |
| 2-21-032 | N—Ph | N—Ph | b3 | b3 | a4 |
| 2-21-033 | N—Ph | N—Ph | H | H | a5 |
| 2-21-034 | N—Ph | N—Ph | t-bu | t-bu | a5 |
| 2-21-035 | N—Ph | N—Ph | methyl | methyl | a5 |
| 2-21-036 | N—Ph | N—Ph | b1 | b1 | a5 |
| 2-21-037 | N—Ph | N—Ph | b2 | b2 | a5 |
| 2-21-038 | N—Ph | N—Ph | b3 | b3 | a5 |
| 2-21-039 | N—Ph | N—Ph | H | H | a6 |
| 2-21-040 | N—Ph | N—Ph | t-bu | t-bu | a6 |
| 2-21-041 | N—Ph | N—Ph | methyl | methyl | a6 |
| 2-21-042 | N—Ph | N—Ph | b1 | b1 | a6 |
| 2-21-043 | N—Ph | N—Ph | b2 | b2 | a6 |
| 2-21-044 | N—Ph | N—Ph | b3 | b3 | a6 |
| 2-21-045 | N—Ph | N—Ph | H | H | b1 |
| 2-21-046 | N—Ph | N—Ph | t-bu | t-bu | b1 |
| 2-21-047 | N—Ph | N—Ph | methyl | methyl | b1 |
| 2-21-048 | N—Ph | N—Ph | b1 | b1 | b1 |
| 2-21-049 | N—Ph | N—Ph | b2 | b2 | b1 |
| 2-21-050 | N—Ph | N—Ph | b3 | b3 | b1 |
| 2-21-051 | N—Ph | N—Ph | H | H | b2 |
| 2-21-052 | N—Ph | N—Ph | t-bu | t-bu | b2 |
| 2-21-053 | N—Ph | N—Ph | methyl | methyl | b2 |
| 2-21-054 | N—Ph | N—Ph | b1 | b1 | b2 |
| 2-21-055 | N—Ph | N—Ph | b2 | b2 | b2 |
| 2-21-056 | N—Ph | N—Ph | b3 | b3 | b2 |
| 2-21-057 | N—Ph | N—Ph | H | H | b3 |
| 2-21-058 | N—Ph | N—Ph | t-bu | t-bu | b3 |
| 2-21-059 | N—Ph | N—Ph | methyl | methyl | b3 |
| 2-21-060 | N—Ph | N—Ph | b1 | b1 | b3 |
| 2-21-061 | N—Ph | N—Ph | b2 | b2 | b3 |
| 2-21-062 | N—Ph | N—Ph | b3 | b3 | b3 |
| 2-21-063 | N—Ph | N—Ph | H | H | b4 |
| 2-21-064 | N—Ph | N—Ph | t-bu | t-bu | b4 |
| 2-21-065 | N—Ph | N—Ph | methyl | methyl | b4 |
| 2-21-066 | N—Ph | N—Ph | b1 | b1 | b4 |
| 2-21-067 | N—Ph | N—Ph | b2 | b2 | b4 |
| 2-21-068 | N—Ph | N—Ph | b3 | b3 | b4 |
| 2-21-069 | N—Ph | N—Ph | H | H | b5 |
| 2-21-070 | N—Ph | N—Ph | t-bu | t-bu | b5 |
| 2-21-071 | N—Ph | N—Ph | methyl | methyl | b5 |
| 2-21-072 | N—Ph | N—Ph | b1 | b1 | b5 |
| 2-21-073 | N—Ph | N—Ph | b2 | b2 | b5 |
| 2-21-074 | N—Ph | N—Ph | b3 | b3 | b5 |
| 2-21-075 | N—Ph | N—Ph | H | H | b6 |
| 2-21-076 | N—Ph | N—Ph | t-bu | t-bu | b6 |
| 2-21-077 | N—Ph | N—Ph | methyl | methyl | b6 |
| 2-21-078 | N—Ph | N—Ph | b1 | b1 | b6 |
| 2-21-079 | N—Ph | N—Ph | b2 | b2 | b6 |
| 2-21-080 | N—Ph | N—Ph | b3 | b3 | b6 |
| 2-21-081 | N—Ph | N—Ph | H | H | b7 |
| 2-21-082 | N—Ph | N—Ph | t-bu | t-bu | b7 |
| 2-21-083 | N—Ph | N—Ph | methyl | methyl | b7 |
| 2-21-084 | N—Ph | N—Ph | b1 | b1 | b7 |
| 2-21-085 | N—Ph | N—Ph | b2 | b2 | b7 |
| 2-21-086 | N—Ph | N—Ph | b3 | b3 | b7 |
| 2-21-087 | N—Ph | N—Ph | H | H | b8 |
| 2-21-088 | N—Ph | N—Ph | t-bu | t-bu | b8 |
| 2-21-089 | N—Ph | N—Ph | methyl | methyl | b8 |
| 2-21-090 | N—Ph | N—Ph | b1 | b1 | b8 |
| 2-21-091 | N—Ph | N—Ph | b2 | b2 | b8 |
| 2-21-092 | N—Ph | N—Ph | b3 | b3 | b8 |
| 2-21-093 | N—Ph | N—Ph | H | H | b9 |
| 2-21-094 | N—Ph | N—Ph | t-bu | t-bu | b9 |
| 2-21-095 | N—Ph | N—Ph | methyl | methyl | b9 |
| 2-21-096 | N—Ph | N—Ph | b1 | b1 | b9 |
| 2-21-097 | N—Ph | N—Ph | b2 | b2 | b9 |
| 2-21-098 | N—Ph | N—Ph | b3 | b3 | b9 |
| 2-21-099 | N—Ph | N—Ph | H | H | b10 |
| 2-21-100 | N—Ph | N—Ph | t-bu | t-bu | b10 |
| 2-21-101 | N—Ph | N—Ph | methyl | methyl | b10 |
| 2-21-102 | N—Ph | N—Ph | b1 | b1 | b10 |
| 2-21-103 | N—Ph | N—Ph | b2 | b2 | b10 |
| 2-21-104 | N—Ph | N—Ph | b3 | b3 | b10 |
| 2-21-105 | N—Ph | N—Ph | H | H | H |
| 2-21-106 | N—Ph | N—Ph | t-bu | t-bu | H |
| 2-21-107 | N—Ph | N—Ph | methyl | methyl | H |
| 2-21-108 | N—Ph | N—Ph | b1 | b1 | H |
| 2-21-109 | N—Ph | N—Ph | b2 | b2 | H |
| 2-21-110 | N—Ph | N—Ph | b3 | b3 | H |
| 2-21-111 | N—Ph | N—Ph | H | H | H |
| 2-21-112 | N—Ph | N—Ph | t-bu | t-bu | H |
| 2-21-113 | N—Ph | N—Ph | methyl | methyl | H |
| 2-21-114 | N—Ph | N—Ph | b1 | b1 | H |
| 2-21-115 | N—Ph | N—Ph | b2 | b2 | H |
| 2-21-116 | N—Ph | N—Ph | b3 | b3 | H |
| 2-21-117 | N—Ph | N—Ph | H | H | H |
| 2-21-118 | N—Ph | N—Ph | t-bu | t-bu | H |
| 2-21-119 | N—Ph | N—Ph | methyl | methyl | H |
| 2-21-120 | N—Ph | N—Ph | b1 | b1 | H |
| 2-21-121 | N—Ph | N—Ph | b2 | b2 | H |
| 2-21-122 | N—Ph | N—Ph | b3 | b3 | H |
| 2-21-123 | N—Ph | N—Ph | H | H | H |
| 2-21-124 | N—Ph | N—Ph | t-bu | t-bu | H |
| 2-21-125 | N—Ph | N—Ph | methyl | methyl | H |
| 2-21-126 | N—Ph | N—Ph | b1 | b1 | H |
| 2-21-127 | N—Ph | N—Ph | b2 | b2 | H |
| 2-21-128 | N—Ph | N—Ph | b3 | b3 | H |
| 2-21-129 | N—Ph | N—Ph | H | H | H |
| 2-21-130 | N—Ph | N—Ph | t-bu | t-bu | H |
| 2-21-131 | N—Ph | N—Ph | methyl | methyl | H |
| 2-21-132 | N—Ph | N—Ph | b1 | b1 | H |
| 2-21-133 | N—Ph | N—Ph | b2 | b2 | H |
| 2-21-134 | N—Ph | N—Ph | b3 | b3 | H |
| 2-21-135 | N—Ph | N—Ph | H | H | H |
| 2-21-136 | N—Ph | N—Ph | t-bu | t-bu | H |
| 2-21-137 | N—Ph | N—Ph | methyl | methyl | H |
| 2-21-138 | N—Ph | N—Ph | b1 | b1 | H |
| 2-21-139 | N—Ph | N—Ph | b2 | b2 | H |
| 2-21-140 | N—Ph | N—Ph | b3 | b3 | H |
| 2-21-141 | N—Ph | N—Ph | H | H | H |
| 2-21-142 | N—Ph | N—Ph | t-bu | t-bu | H |
| 2-21-143 | N—Ph | N—Ph | methyl | methyl | H |
| 2-21-144 | N—Ph | N—Ph | b1 | b1 | H |
| 2-21-145 | N—Ph | N—Ph | b2 | b2 | H |
| 2-21-146 | N—Ph | N—Ph | b3 | b3 | H |
| 2-21-147 | N—Ph | N—Ph | H | H | H |
| 2-21-148 | N—Ph | N—Ph | t-bu | t-bu | H |
| 2-21-149 | N—Ph | N—Ph | methyl | methyl | H |
| 2-21-150 | N—Ph | N—Ph | b1 | b1 | H |
| 2-21-151 | N—Ph | N—Ph | b2 | b2 | H |
| 2-21-152 | N—Ph | N—Ph | b3 | b3 | H |
| 2-21-153 | N—Ph | N—Ph | H | H | H |
| 2-21-154 | N—Ph | N—Ph | t-bu | t-bu | H |
| 2-21-155 | N—Ph | N—Ph | methyl | methyl | H |
| 2-21-156 | N—Ph | N—Ph | b1 | b1 | H |
| 2-21-157 | N—Ph | N—Ph | b2 | b2 | H |
| 2-21-158 | N—Ph | N—Ph | b3 | b3 | H |
| 2-21-159 | N—Ph | N—Ph | H | H | H |
| 2-21-160 | N—Ph | N—Ph | t-bu | t-bu | H |
| 2-21-161 | N—Ph | N—Ph | methyl | methyl | H |
| 2-21-162 | N—Ph | N—Ph | b1 | b1 | H |
| 2-21-163 | N—Ph | N—Ph | b2 | b2 | H |
| 2-21-164 | N—Ph | N—Ph | b3 | b3 | H |
| 2-21-165 | N—Ph | N—Ph | H | H | H |
| 2-21-166 | N—Ph | N—Ph | t-bu | t-bu | H |
| 2-21-167 | N—Ph | N—Ph | methyl | methyl | H |
| 2-21-168 | N—Ph | N—Ph | b1 | b1 | H |
| 2-21-169 | N—Ph | N—Ph | b2 | b2 | H |
| 2-21-170 | N—Ph | N—Ph | b3 | b3 | H |
| 2-21-171 | N—Ph | N—Ph | H | H | H |
| 2-21-172 | N—Ph | N—Ph | t-bu | t-bu | H |
| 2-21-173 | N—Ph | N—Ph | methyl | methyl | H |
| 2-21-174 | N—Ph | N—Ph | b1 | b1 | H |
| 2-21-175 | N—Ph | N—Ph | b2 | b2 | H |
| 2-21-176 | N—Ph | N—Ph | b3 | b3 | H |
| 2-21-177 | N—Ph | N—Ph | H | H | H |
| 2-21-178 | N—Ph | N—Ph | t-bu | t-bu | H |
| 2-21-179 | N—Ph | N—Ph | methyl | methyl | H |
| 2-21-180 | N—Ph | N—Ph | b1 | b1 | H |
| 2-21-181 | N—Ph | N—Ph | b2 | b2 | H |
| 2-21-182 | N—Ph | N—Ph | b3 | b3 | H |
| 2-21-183 | N—Ph | N—Ph | H | H | H |
| 2-21-184 | N—Ph | N—Ph | t-bu | t-bu | H |
| 2-21-185 | N—Ph | N—Ph | methyl | methyl | H |
| 2-21-186 | N—Ph | N—Ph | b1 | b1 | H |
| 2-21-187 | N—Ph | N—Ph | b2 | b2 | H |
| 2-21-188 | N—Ph | N—Ph | b3 | b3 | H |
| 2-21-189 | N—Ph | N—Ph | H | H | H |
| 2-21-190 | N—Ph | N—Ph | t-bu | t-bu | H |
| 2-21-191 | N—Ph | N—Ph | methyl | methyl | H |
| 2-21-192 | N—Ph | N—Ph | b1 | b1 | H |
| 2-21-193 | N—Ph | N—Ph | b2 | b2 | H |
| 2-21-194 | N—Ph | N—Ph | b3 | b3 | H |
| 2-21-195 | N—Ph | N—Ph | H | H | H |
| 2-21-196 | N—Ph | N—Ph | t-bu | t-bu | H |
| 2-21-197 | N—Ph | N—Ph | methyl | methyl | H |
| 2-21-198 | N—Ph | N—Ph | b1 | b1 | H |
| 2-21-199 | N—Ph | N—Ph | b2 | b2 | H |
| 2-21-200 | N—Ph | N—Ph | b3 | b3 | H |
| 2-21-201 | N—Ph | N—Ph | H | H | a1 |
| 2-21-202 | N—Ph | N—Ph | t-bu | t-bu | a1 |
| 2-21-203 | N—Ph | N—Ph | methyl | methyl | a1 |
| 2-21-204 | N—Ph | N—Ph | b1 | b1 | a1 |
| 2-21-205 | N—Ph | N—Ph | b2 | b2 | a1 |
| 2-21-206 | N—Ph | N—Ph | b3 | b3 | a1 |
| 2-21-207 | N—Ph | N—Ph | H | H | a2 |
| 2-21-208 | N—Ph | N—Ph | t-bu | t-bu | a2 |
| 2-21-209 | N—Ph | N—Ph | methyl | methyl | a2 |
| 2-21-210 | N—Ph | N—Ph | b1 | b1 | a2 |
| 2-21-211 | N—Ph | N—Ph | b2 | b2 | a2 |
| 2-21-212 | N—Ph | N—Ph | b3 | b3 | a2 |
| 2-21-213 | N—Ph | N—Ph | H | H | a3 |
| 2-21-214 | N—Ph | N—Ph | t-bu | t-bu | a3 |
| 2-21-215 | N—Ph | N—Ph | methyl | methyl | a3 |
| 2-21-216 | N—Ph | N—Ph | b1 | b1 | a3 |
| 2-21-217 | N—Ph | N—Ph | b2 | b2 | a3 |
| 2-21-218 | N—Ph | N—Ph | b3 | b3 | a3 |
| 2-21-219 | N—Ph | N—Ph | H | H | b1 |
| 2-21-220 | N—Ph | N—Ph | t-bu | t-bu | b1 |
| 2-21-221 | N—Ph | N—Ph | methyl | methyl | b1 |
| 2-21-222 | N—Ph | N—Ph | b1 | b1 | b1 |
| 2-21-223 | N—Ph | N—Ph | b2 | b2 | b1 |
| 2-21-224 | N—Ph | N—Ph | b3 | b3 | b1 |
| 2-21-225 | N—Ph | N—Ph | H | H | b2 |
| 2-21-226 | N—Ph | N—Ph | t-bu | t-bu | b2 |
| 2-21-227 | N—Ph | N—Ph | methyl | methyl | b2 |
| 2-21-228 | N—Ph | N—Ph | b1 | b1 | b2 |
| 2-21-229 | N—Ph | N—Ph | b2 | b2 | b2 |
| 2-21-230 | N—Ph | N—Ph | b3 | b3 | b2 |
| 2-21-231 | N—Ph | N—Ph | H | H | b3 |
| 2-21-232 | N—Ph | N—Ph | t-bu | t-bu | b3 |
| 2-21-233 | N—Ph | N—Ph | methyl | methyl | b3 |
| 2-21-234 | N—Ph | N—Ph | b1 | b1 | b3 |
| 2-21-235 | N—Ph | N—Ph | b2 | b2 | b3 |
| 2-21-236 | N—Ph | N—Ph | b3 | b3 | b3 |
| 2-21-237 | N—Ph | N—Ph | H | H | b5 |
| 2-21-238 | N—Ph | N—Ph | t-bu | t-bu | b5 |
| 2-21-239 | N—Ph | N—Ph | methyl | methyl | b5 |
| 2-21-240 | N—Ph | N—Ph | b1 | b1 | b5 |
| 2-21-241 | N—Ph | N—Ph | b2 | b2 | b5 |
| 2-21-242 | N—Ph | N—Ph | b3 | b3 | b5 |
| 2-21-243 | N—Ph | N—Ph | H | H | b6 |
| 2-21-244 | N—Ph | N—Ph | t-bu | t-bu | b6 |
| 2-21-245 | N—Ph | N—Ph | methyl | methyl | b6 |
| 2-21-246 | N—Ph | N—Ph | b1 | b1 | b6 |
| 2-21-247 | N—Ph | N—Ph | b2 | b2 | b6 |
| 2-21-248 | N—Ph | N—Ph | b3 | b3 | b6 |
| 2-21-249 | N—Ph | N—Ph | H | H | b7 |
| 2-21-250 | N—Ph | N—Ph | t-bu | t-bu | b7 |
| 2-21-251 | N—Ph | N—Ph | methyl | methyl | b7 |
| 2-21-252 | N—Ph | N—Ph | b1 | b1 | b7 |
| 2-21-253 | N—Ph | N—Ph | b2 | b2 | b7 |
| 2-21-254 | N—Ph | N—Ph | b3 | b3 | b7 |
| 2-21-255 | N-tolyl | N-tolyl | H | H | H |
| 2-21-256 | N-tolyl | N-tolyl | t-bu | t-bu | H |
| 2-21-257 | N-tolyl | N-tolyl | methyl | methyl | H |
| 2-21-258 | N-tolyl | N-tolyl | t-bu | t-bu | t-bu |
| 2-21-259 | N-tolyl | N-tolyl | methyl | methyl | methyl |
| 2-21-260 | N-tolyl | N-tolyl | b1 | b1 | H |
| 2-21-261 | N-tolyl | N-tolyl | b2 | b2 | H |
| 2-21-262 | N-tolyl | N-tolyl | b3 | b3 | H |
| 2-21-263 | N-tolyl | N-tolyl | H | H | a1 |
| 2-21-264 | N-tolyl | N-tolyl | t-bu | t-bu | a1 |
| 2-21-265 | N-tolyl | N-tolyl | methyl | methyl | a1 |
| 2-21-266 | N-tolyl | N-tolyl | b1 | b1 | a1 |
| 2-21-267 | N-tolyl | N-tolyl | b2 | b2 | a1 |
| 2-21-268 | N-tolyl | N-tolyl | b3 | b3 | a1 |
| 2-21-269 | N-tolyl | N-tolyl | H | H | a2 |
| 2-21-270 | N-tolyl | N-tolyl | t-bu | t-bu | a2 |
| 2-21-271 | N-tolyl | N-tolyl | methyl | methyl | a2 |
| 2-21-272 | N-tolyl | N-tolyl | b1 | b1 | a2 |
| 2-21-273 | N-tolyl | N-tolyl | b2 | b2 | a2 |
| 2-21-274 | N-tolyl | N-tolyl | b3 | b3 | a2 |
| 2-21-275 | N-tolyl | N-tolyl | H | H | a3 |
| 2-21-276 | N-tolyl | N-tolyl | t-bu | t-bu | a3 |
| 2-21-277 | N-tolyl | N-tolyl | methyl | methyl | a3 |
| 2-21-278 | N-tolyl | N-tolyl | b1 | b1 | a3 |
| 2-21-279 | N-tolyl | N-tolyl | b2 | b2 | a3 |
| 2-21-280 | N-tolyl | N-tolyl | b3 | b3 | a3 |
| 2-21-281 | N-tolyl | N-tolyl | H | H | a4 |
| 2-21-282 | N-tolyl | N-tolyl | t-bu | t-bu | a4 |
| 2-21-283 | N-tolyl | N-tolyl | methyl | methyl | a4 |
| 2-21-284 | N-tolyl | N-tolyl | b1 | b1 | a4 |
| 2-21-285 | N-tolyl | N-tolyl | b2 | b2 | a4 |
| 2-21-286 | N-tolyl | N-tolyl | b3 | b3 | a4 |
| 2-21-287 | N-tolyl | N-tolyl | H | H | a5 |
| 2-21-288 | N-tolyl | N-tolyl | t-bu | t-bu | a5 |
| 2-21-289 | N-tolyl | N-tolyl | methyl | methyl | a5 |
| 2-21-290 | N-tolyl | N-tolyl | b1 | b1 | a5 |
| 2-21-291 | N-tolyl | N-tolyl | b2 | b2 | a5 |
| 2-21-292 | N-tolyl | N-tolyl | b3 | b3 | a5 |
| 2-21-293 | N-tolyl | N-tolyl | H | H | a6 |
| 2-21-294 | N-tolyl | N-tolyl | t-bu | t-bu | a6 |
| 2-21-295 | N-tolyl | N-tolyl | methyl | methyl | a6 |
| 2-21-296 | N-tolyl | N-tolyl | b1 | b1 | a6 |
| 2-21-297 | N-tolyl | N-tolyl | b2 | b2 | a6 |
| 2-21-298 | N-tolyl | N-tolyl | b3 | b3 | a6 |
| 2-21-299 | N-tolyl | N-tolyl | H | H | b1 |
| 2-21-300 | N-tolyl | N-tolyl | t-bu | t-bu | b1 |
| 2-21-301 | N-tolyl | N-tolyl | methyl | methyl | b1 |
| 2-21-302 | N-tolyl | N-tolyl | b1 | b1 | b1 |
| 2-21-303 | N-tolyl | N-tolyl | b2 | b2 | b1 |
| 2-21-304 | N-tolyl | N-tolyl | b3 | b3 | b1 |
| 2-21-305 | N-tolyl | N-tolyl | H | H | b2 |
| 2-21-306 | N-tolyl | N-tolyl | t-bu | t-bu | b2 |
| 2-21-307 | N-tolyl | N-tolyl | methyl | methyl | b2 |
| 2-21-308 | N-tolyl | N-tolyl | b1 | b1 | b2 |
| 2-21-309 | N-tolyl | N-tolyl | b2 | b2 | b2 |
| 2-21-310 | N-tolyl | N-tolyl | b3 | b3 | b2 |
| 2-21-311 | N-tolyl | N-tolyl | H | H | b3 |
| 2-21-312 | N-tolyl | N-tolyl | t-bu | t-bu | b3 |
| 2-21-313 | N-tolyl | N-tolyl | methyl | methyl | b3 |
| 2-21-314 | N-tolyl | N-tolyl | b1 | b1 | b3 |
| 2-21-315 | N-tolyl | N-tolyl | b2 | b2 | b3 |
| 2-21-316 | N-tolyl | N-tolyl | b3 | b3 | b3 |
| 2-21-317 | N-tolyl | N-tolyl | H | H | b4 |
| 2-21-318 | N-tolyl | N-tolyl | t-bu | t-bu | b4 |
| 2-21-319 | N-tolyl | N-tolyl | methyl | methyl | b4 |
| 2-21-320 | N-tolyl | N-tolyl | b1 | b1 | b4 |
| 2-21-321 | N-tolyl | N-tolyl | b2 | b2 | b4 |
| 2-21-322 | N-tolyl | N-tolyl | b3 | b3 | b4 |
| 2-21-323 | N-tolyl | N-tolyl | H | H | b5 |
| 2-21-324 | N-tolyl | N-tolyl | t-bu | t-bu | b5 |
| 2-21-325 | N-tolyl | N-tolyl | methyl | methyl | b5 |
| 2-21-326 | N-tolyl | N-tolyl | b1 | b1 | b5 |
| 2-21-327 | N-tolyl | N-tolyl | b2 | b2 | b5 |
| 2-21-328 | N-tolyl | N-tolyl | b3 | b3 | b5 |
| 2-21-329 | N-tolyl | N-tolyl | H | H | b6 |
| 2-21-330 | N-tolyl | N-tolyl | t-bu | t-bu | b6 |
| 2-21-331 | N-tolyl | N-tolyl | methyl | methyl | b6 |
| 2-21-332 | N-tolyl | N-tolyl | b1 | b1 | b6 |
| 2-21-333 | N-tolyl | N-tolyl | b2 | b2 | b6 |
| 2-21-334 | N-tolyl | N-tolyl | b3 | b3 | b6 |
| 2-21-335 | N-tolyl | N-tolyl | H | H | b7 |
| 2-21-336 | N-tolyl | N-tolyl | t-bu | t-bu | b7 |
| 2-21-337 | N-tolyl | N-tolyl | methyl | methyl | b7 |
| 2-21-338 | N-tolyl | N-tolyl | b1 | b1 | b7 |
| 2-21-339 | N-tolyl | N-tolyl | b2 | b2 | b7 |
| 2-21-340 | N-tolyl | N-tolyl | b3 | b3 | b7 |
| 2-21-341 | N-tolyl | N-tolyl | H | H | b8 |
| 2-21-342 | N-tolyl | N-tolyl | t-bu | t-bu | b8 |
| 2-21-343 | N-tolyl | N-tolyl | methyl | methyl | b8 |
| 2-21-344 | N-tolyl | N-tolyl | b1 | b1 | b8 |
| 2-21-345 | N-tolyl | N-tolyl | b2 | b2 | b8 |
| 2-21-346 | N-tolyl | N-tolyl | b3 | b3 | b8 |
| 2-21-347 | N-tolyl | N-tolyl | H | H | b9 |
| 2-21-348 | N-tolyl | N-tolyl | t-bu | t-bu | b9 |
| 2-21-349 | N-tolyl | N-tolyl | methyl | methyl | b9 |
| 2-21-350 | N-tolyl | N-tolyl | b1 | b1 | b9 |
| 2-21-351 | N-tolyl | N-tolyl | b2 | b2 | b9 |
| 2-21-352 | N-tolyl | N-tolyl | b3 | b3 | b9 |
| 2-21-353 | N-tolyl | N-tolyl | H | H | b10 |
| 2-21-354 | N-tolyl | N-tolyl | t-bu | t-bu | b10 |
| 2-21-355 | N-tolyl | N-tolyl | methyl | methyl | b10 |
| 2-21-356 | N-tolyl | N-tolyl | b1 | b1 | b10 |
| 2-21-357 | N-tolyl | N-tolyl | b2 | b2 | b10 |
| 2-21-358 | N-tolyl | N-tolyl | b3 | b3 | b10 |
| 2-21-359 | N-tolyl | N-tolyl | H | H | H |
| 2-21-360 | N-tolyl | N-tolyl | t-bu | t-bu | H |
| 2-21-361 | N-tolyl | N-tolyl | methyl | methyl | H |
| 2-21-362 | N-tolyl | N-tolyl | b1 | b1 | H |
| 2-21-363 | N-tolyl | N-tolyl | b2 | b2 | H |
| 2-21-364 | N-tolyl | N-tolyl | b3 | b3 | H |
| 2-21-365 | N-tolyl | N-tolyl | H | H | H |
| 2-21-366 | N-tolyl | N-tolyl | t-bu | t-bu | H |
| 2-21-367 | N-tolyl | N-tolyl | methyl | methyl | H |
| 2-21-368 | N-tolyl | N-tolyl | b1 | b1 | H |
| 2-21-369 | N-tolyl | N-tolyl | b2 | b2 | H |
| 2-21-370 | N-tolyl | N-tolyl | b3 | b3 | H |
| 2-21-371 | N-tolyl | N-tolyl | H | H | H |
| 2-21-372 | N-tolyl | N-tolyl | t-bu | t-bu | H |
| 2-21-373 | N-tolyl | N-tolyl | methyl | methyl | H |
| 2-21-374 | N-tolyl | N-tolyl | b1 | b1 | H |
| 2-21-375 | N-tolyl | N-tolyl | b2 | b2 | H |
| 2-21-376 | N-tolyl | N-tolyl | b3 | b3 | H |
| 2-21-377 | N-tolyl | N-tolyl | H | H | H |
| 2-21-378 | N-tolyl | N-tolyl | t-bu | t-bu | H |
| 2-21-379 | N-tolyl | N-tolyl | methyl | methyl | H |
| 2-21-380 | N-tolyl | N-tolyl | b1 | b1 | H |
| 2-21-381 | N-tolyl | N-tolyl | b2 | b2 | H |
| 2-21-382 | N-tolyl | N-tolyl | b3 | b3 | H |
| 2-21-383 | N-tolyl | N-tolyl | H | H | H |
| 2-21-384 | N-tolyl | N-tolyl | t-bu | t-bu | H |
| 2-21-385 | N-tolyl | N-tolyl | methyl | methyl | H |
| 2-21-386 | N-tolyl | N-tolyl | b1 | b1 | H |
| 2-21-387 | N-tolyl | N-tolyl | b2 | b2 | H |
| 2-21-388 | N-tolyl | N-tolyl | b3 | b3 | H |
| 2-21-389 | N-tolyl | N-tolyl | H | H | H |
| 2-21-390 | N-tolyl | N-tolyl | t-bu | t-bu | H |
| 2-21-391 | N-tolyl | N-tolyl | methyl | methyl | H |
| 2-21-392 | N-tolyl | N-tolyl | b1 | b1 | H |
| 2-21-393 | N-tolyl | N-tolyl | b2 | b2 | H |
| 2-21-394 | N-tolyl | N-tolyl | b3 | b3 | H |
| 2-21-395 | N-tolyl | N-tolyl | H | H | H |
| 2-21-396 | N-tolyl | N-tolyl | t-bu | t-bu | H |
| 2-21-397 | N-tolyl | N-tolyl | methyl | methyl | H |
| 2-21-398 | N-tolyl | N-tolyl | b1 | b1 | H |
| 2-21-399 | N-tolyl | N-tolyl | b2 | b2 | H |
| 2-21-400 | N-tolyl | N-tolyl | b3 | b3 | H |
| 2-21-401 | N-tolyl | N-tolyl | H | H | H |
| 2-21-402 | N-tolyl | N-tolyl | t-bu | t-bu | H |
| 2-21-403 | N-tolyl | N-tolyl | methyl | methyl | H |
| 2-21-404 | N-tolyl | N-tolyl | b1 | b1 | H |
| 2-21-405 | N-tolyl | N-tolyl | b2 | b2 | H |
| 2-21-406 | N-tolyl | N-tolyl | b3 | b3 | H |
| 2-21-407 | N-tolyl | N-tolyl | H | H | H |
| 2-21-408 | N-tolyl | N-tolyl | t-bu | t-bu | H |
| 2-21-409 | N-tolyl | N-tolyl | methyl | methyl | H |
| 2-21-410 | N-tolyl | N-tolyl | b1 | b1 | H |
| 2-21-411 | N-tolyl | N-tolyl | b2 | b2 | H |
| 2-21-412 | N-tolyl | N-tolyl | b3 | b3 | H |
| 2-21-413 | N-tolyl | N-tolyl | H | H | H |
| 2-21-414 | N-tolyl | N-tolyl | t-bu | t-bu | H |
| 2-21-415 | N-tolyl | N-tolyl | methyl | methyl | H |
| 2-21-416 | N-tolyl | N-tolyl | b1 | b1 | H |
| 2-21-417 | N-tolyl | N-tolyl | b2 | b2 | H |
| 2-21-418 | N-tolyl | N-tolyl | b3 | b3 | H |
| 2-21-419 | N-tolyl | N-tolyl | H | H | H |
| 2-21-420 | N-tolyl | N-tolyl | t-bu | t-bu | H |
| 2-21-421 | N-tolyl | N-tolyl | methyl | methyl | H |
| 2-21-422 | N-tolyl | N-tolyl | b1 | b1 | H |
| 2-21-423 | N-tolyl | N-tolyl | b2 | b2 | H |
| 2-21-424 | N-tolyl | N-tolyl | b3 | b3 | H |
| 2-21-425 | N-tolyl | N-tolyl | H | H | H |
| 2-21-426 | N-tolyl | N-tolyl | t-bu | t-bu | H |
| 2-21-427 | N-tolyl | N-tolyl | methyl | methyl | H |
| 2-21-428 | N-tolyl | N-tolyl | b1 | b1 | H |
| 2-21-429 | N-tolyl | N-tolyl | b2 | b2 | H |
| 2-21-430 | N-tolyl | N-tolyl | b3 | b3 | H |
| 2-21-431 | N-tolyl | N-tolyl | H | H | H |
| 2-21-432 | N-tolyl | N-tolyl | t-bu | t-bu | H |
| 2-21-433 | N-tolyl | N-tolyl | methyl | methyl | H |
| 2-21-434 | N-tolyl | N-tolyl | b1 | b1 | H |
| 2-21-435 | N-tolyl | N-tolyl | b2 | b2 | H |
| 2-21-436 | N-tolyl | N-tolyl | b3 | b3 | H |
| 2-21-437 | N-tolyl | N-tolyl | H | H | H |
| 2-21-438 | N-tolyl | N-tolyl | t-bu | t-bu | H |
| 2-21-439 | N-tolyl | N-tolyl | methyl | methyl | H |
| 2-21-440 | N-tolyl | N-tolyl | b1 | b1 | H |
| 2-21-441 | N-tolyl | N-tolyl | b2 | b2 | H |
| 2-21-442 | N-tolyl | N-tolyl | b3 | b3 | H |
| 2-21-443 | N-tolyl | N-tolyl | H | H | H |
| 2-21-444 | N-tolyl | N-tolyl | t-bu | t-bu | H |
| 2-21-445 | N-tolyl | N-tolyl | methyl | methyl | H |
| 2-21-446 | N-tolyl | N-tolyl | b1 | b1 | H |
| 2-21-447 | N-tolyl | N-tolyl | b2 | b2 | H |
| 2-21-448 | N-tolyl | N-tolyl | b3 | b3 | H |
| 2-21-449 | N-tolyl | N-tolyl | H | H | H |
| 2-21-450 | N-tolyl | N-tolyl | t-bu | t-bu | H |
| 2-21-451 | N-tolyl | N-tolyl | methyl | methyl | H |
| 2-21-452 | N-tolyl | N-tolyl | b1 | b1 | H |
| 2-21-453 | N-tolyl | N-tolyl | b2 | b2 | H |
| 2-21-454 | N-tolyl | N-tolyl | b3 | b3 | H |
| 2-21-455 | N-tolyl | N-tolyl | H | H | a1 |
| 2-21-456 | N-tolyl | N-tolyl | t-bu | t-bu | a1 |
| 2-21-457 | N-tolyl | N-tolyl | methyl | methyl | a1 |
| 2-21-458 | N-tolyl | N-tolyl | b1 | b1 | a1 |
| 2-21-459 | N-tolyl | N-tolyl | b2 | b2 | a1 |
| 2-21-460 | N-tolyl | N-tolyl | b3 | b3 | a1 |
| 2-21-461 | N-tolyl | N-tolyl | H | H | a2 |
| 2-21-462 | N-tolyl | N-tolyl | t-bu | t-bu | a2 |
| 2-21-463 | N-tolyl | N-tolyl | methyl | methyl | a2 |
| 2-21-464 | N-tolyl | N-tolyl | b1 | b1 | a2 |
| 2-21-465 | N-tolyl | N-tolyl | b2 | b2 | a2 |
| 2-21-466 | N-tolyl | N-tolyl | b3 | b3 | a2 |
| 2-21-467 | N-tolyl | N-tolyl | H | H | a3 |
| 2-21-468 | N-tolyl | N-tolyl | t-bu | t-bu | a3 |
| 2-21-469 | N-tolyl | N-tolyl | methyl | methyl | a3 |
| 2-21-470 | N-tolyl | N-tolyl | b1 | b1 | a3 |
| 2-21-471 | N-tolyl | N-tolyl | b2 | b2 | a3 |
| 2-21-472 | N-tolyl | N-tolyl | b3 | b3 | a3 |
| 2-21-473 | N-tolyl | N-tolyl | H | H | b1 |
| 2-21-474 | N-tolyl | N-tolyl | t-bu | t-bu | b1 |
| 2-21-475 | N-tolyl | N-tolyl | methyl | methyl | b1 |
| 2-21-476 | N-tolyl | N-tolyl | b1 | b1 | b1 |
| 2-21-477 | N-tolyl | N-tolyl | b2 | b2 | b1 |
| 2-21-478 | N-tolyl | N-tolyl | b3 | b3 | b1 |
| 2-21-479 | N-tolyl | N-tolyl | H | H | b2 |
| 2-21-480 | N-tolyl | N-tolyl | t-bu | t-bu | b2 |
| 2-21-481 | N-tolyl | N-tolyl | methyl | methyl | b2 |
| 2-21-482 | N-tolyl | N-tolyl | b1 | b1 | b2 |
| 2-21-483 | N-tolyl | N-tolyl | b2 | b2 | b2 |
| 2-21-484 | N-tolyl | N-tolyl | b3 | b3 | b2 |
| 2-21-485 | N-tolyl | N-tolyl | H | H | b3 |
| 2-21-486 | N-tolyl | N-tolyl | t-bu | t-bu | b3 |
| 2-21-487 | N-tolyl | N-tolyl | methyl | methyl | b3 |
| 2-21-488 | N-tolyl | N-tolyl | b1 | b1 | b3 |
| 2-21-489 | N-tolyl | N-tolyl | b2 | b2 | b3 |
| 2-21-490 | N-tolyl | N-tolyl | b3 | b3 | b3 |
| 2-21-491 | N-tolyl | N-tolyl | H | H | b5 |
| 2-21-492 | N-tolyl | N-tolyl | t-bu | t-bu | b5 |
| 2-21-493 | N-tolyl | N-tolyl | methyl | methyl | b5 |
| 2-21-494 | N-tolyl | N-tolyl | b1 | b1 | b5 |
| 2-21-495 | N-tolyl | N-tolyl | b2 | b2 | b5 |
| 2-21-496 | N-tolyl | N-tolyl | b3 | b3 | b5 |
| 2-21-497 | N-tolyl | N-tolyl | H | H | b6 |
| 2-21-498 | N-tolyl | N-tolyl | t-bu | t-bu | b6 |
| 2-21-499 | N-tolyl | N-tolyl | methyl | methyl | b6 |
| 2-21-500 | N-tolyl | N-tolyl | b1 | b1 | b6 |
| 2-21-501 | N-tolyl | N-tolyl | b2 | b2 | b6 |
| 2-21-502 | N-tolyl | N-tolyl | b3 | b3 | b6 |
| 2-21-503 | N-tolyl | N-tolyl | H | H | b7 |
| 2-21-504 | N-tolyl | N-tolyl | t-bu | t-bu | b7 |
| 2-21-505 | N-tolyl | N-tolyl | methyl | methyl | b7 |
| 2-21-506 | N-tolyl | N-tolyl | b1 | b1 | b7 |
| 2-21-507 | N-tolyl | N-tolyl | b2 | b2 | b7 |
| 2-21-508 | N-tolyl | N-tolyl | b3 | b3 | b7 |
| 2-21-509 | N-Biph | N-Biph | H | H | H |
| 2-21-510 | N-Biph | N-Biph | t-bu | t-bu | H |
| 2-21-511 | N-Biph | N-Biph | methyl | methyl | H |
| 2-21-512 | N-Biph | N-Biph | t-bu | t-bu | t-bu |
| 2-21-513 | N-Biph | N-Biph | methyl | methyl | methyl |
| 2-21-514 | N-Biph | N-Biph | b1 | b1 | H |
| 2-21-515 | N-Biph | N-Biph | b2 | b2 | H |
| 2-21-516 | N-Biph | N-Biph | b3 | b3 | H |
| 2-21-517 | N-Biph | N-Biph | H | H | a1 |
| 2-21-518 | N-Biph | N-Biph | t-bu | t-bu | a1 |
| 2-21-519 | N-Biph | N-Biph | methyl | methyl | a1 |
| 2-21-520 | N-Biph | N-Biph | b1 | b1 | a1 |
| 2-21-521 | N-Biph | N-Biph | b2 | b2 | a1 |
| 2-21-522 | N-Biph | N-Biph | b3 | b3 | a1 |
| 2-21-523 | N-Biph | N-Biph | H | H | a2 |
| 2-21-524 | N-Biph | N-Biph | t-bu | t-bu | a2 |
| 2-21-525 | N-Biph | N-Biph | methyl | methyl | a2 |
| 2-21-526 | N-Biph | N-Biph | b1 | b1 | a2 |
| 2-21-527 | N-Biph | N-Biph | b2 | b2 | a2 |
| 2-21-528 | N-Biph | N-Biph | b3 | b3 | a2 |
| 2-21-529 | N-Biph | N-Biph | H | H | a3 |
| 2-21-530 | N-Biph | N-Biph | t-bu | t-bu | a3 |
| 2-21-531 | N-Biph | N-Biph | methyl | methyl | a3 |
| 2-21-532 | N-Biph | N-Biph | b1 | b1 | a3 |
| 2-21-533 | N-Biph | N-Biph | b2 | b2 | a3 |
| 2-21-534 | N-Biph | N-Biph | b3 | b3 | a3 |
| 2-21-535 | N-Biph | N-Biph | H | H | a4 |
| 2-21-536 | N-Biph | N-Biph | t-bu | t-bu | a4 |
| 2-21-537 | N-Biph | N-Biph | methyl | methyl | a4 |
| 2-21-538 | N-Biph | N-Biph | b1 | b1 | a4 |
| 2-21-539 | N-Biph | N-Biph | b2 | b2 | a4 |
| 2-21-540 | N-Biph | N-Biph | b3 | b3 | a4 |
| 2-21-541 | N-Biph | N-Biph | H | H | a5 |
| 2-21-542 | N-Biph | N-Biph | t-bu | t-bu | a5 |
| 2-21-543 | N-Biph | N-Biph | methyl | methyl | a5 |
| 2-21-544 | N-Biph | N-Biph | b1 | b1 | a5 |
| 2-21-545 | N-Biph | N-Biph | b2 | b2 | a5 |
| 2-21-546 | N-Biph | N-Biph | b3 | b3 | a5 |
| 2-21-547 | N-Biph | N-Biph | H | H | a6 |
| 2-21-548 | N-Biph | N-Biph | t-bu | t-bu | a6 |
| 2-21-549 | N-Biph | N-Biph | methyl | methyl | a6 |
| 2-21-550 | N-Biph | N-Biph | b1 | b1 | a6 |
| 2-21-551 | N-Biph | N-Biph | b2 | b2 | a6 |
| 2-21-552 | N-Biph | N-Biph | b3 | b3 | a6 |
| 2-21-553 | N-Biph | N-Biph | H | H | b1 |
| 2-21-554 | N-Biph | N-Biph | t-bu | t-bu | b1 |
| 2-21-555 | N-Biph | N-Biph | methyl | methyl | b1 |
| 2-21-556 | N-Biph | N-Biph | b1 | b1 | b1 |
| 2-21-557 | N-Biph | N-Biph | b2 | b2 | b1 |
| 2-21-558 | N-Biph | N-Biph | b3 | b3 | b1 |
| 2-21-559 | N-Biph | N-Biph | H | H | b2 |
| 2-21-560 | N-Biph | N-Biph | t-bu | t-bu | b2 |
| 2-21-561 | N-Biph | N-Biph | methyl | methyl | b2 |
| 2-21-562 | N-Biph | N-Biph | b1 | b1 | b2 |
| 2-21-563 | N-Biph | N-Biph | b2 | b2 | b2 |
| 2-21-564 | N-Biph | N-Biph | b3 | b3 | b2 |
| 2-21-565 | N-Biph | N-Biph | H | H | b3 |
| 2-21-566 | N-Biph | N-Biph | t-bu | t-bu | b3 |
| 2-21-567 | N-Biph | N-Biph | methyl | methyl | b3 |
| 2-21-568 | N-Biph | N-Biph | b1 | b1 | b3 |
| 2-21-569 | N-Biph | N-Biph | b2 | b2 | b3 |
| 2-21-570 | N-Biph | N-Biph | b3 | b3 | b3 |
| 2-21-571 | N-Biph | N-Biph | H | H | b4 |
| 2-21-572 | N-Biph | N-Biph | t-bu | t-bu | b4 |
| 2-21-573 | N-Biph | N-Biph | methyl | methyl | b4 |
| 2-21-574 | N-Biph | N-Biph | b1 | b1 | b4 |
| 2-21-575 | N-Biph | N-Biph | b2 | b2 | b4 |
| 2-21-576 | N-Biph | N-Biph | b3 | b3 | b4 |
| 2-21-577 | N-Biph | N-Biph | H | H | b5 |
| 2-21-578 | N-Biph | N-Biph | t-bu | t-bu | b5 |
| 2-21-579 | N-Biph | N-Biph | methyl | methyl | b5 |
| 2-21-580 | N-Biph | N-Biph | b1 | b1 | b5 |
| 2-21-581 | N-Biph | N-Biph | b2 | b2 | b5 |
| 2-21-582 | N-Biph | N-Biph | b3 | b3 | b5 |
| 2-21-583 | N-Biph | N-Biph | H | H | b6 |
| 2-21-584 | N-Biph | N-Biph | t-bu | t-bu | b6 |
| 2-21-585 | N-Biph | N-Biph | methyl | methyl | b6 |
| 2-21-586 | N-Biph | N-Biph | b1 | b1 | b6 |
| 2-21-587 | N-Biph | N-Biph | b2 | b2 | b6 |
| 2-21-588 | N-Biph | N-Biph | b3 | b3 | b6 |
| 2-21-589 | N-Biph | N-Biph | H | H | b7 |
| 2-21-590 | N-Biph | N-Biph | t-bu | t-bu | b7 |
| 2-21-591 | N-Biph | N-Biph | methyl | methyl | b7 |
| 2-21-592 | N-Biph | N-Biph | b1 | b1 | b7 |
| 2-21-593 | N-Biph | N-Biph | b2 | b2 | b7 |
| 2-21-594 | N-Biph | N-Biph | b3 | b3 | b7 |
| 2-21-595 | N-Biph | N-Biph | H | H | b8 |
| 2-21-596 | N-Biph | N-Biph | t-bu | t-bu | b8 |
| 2-21-597 | N-Biph | N-Biph | methyl | methyl | b8 |
| 2-21-598 | N-Biph | N-Biph | b1 | b1 | b8 |
| 2-21-599 | N-Biph | N-Biph | b2 | b2 | b8 |
| 2-21-600 | N-Biph | N-Biph | b3 | b3 | b8 |
| 2-21-601 | N-Biph | N-Biph | H | H | b9 |
| 2-21-602 | N-Biph | N-Biph | t-bu | t-bu | b9 |
| 2-21-603 | N-Biph | N-Biph | methyl | methyl | b9 |
| 2-21-604 | N-Biph | N-Biph | b1 | b1 | b9 |
| 2-21-605 | N-Biph | N-Biph | b2 | b2 | b9 |
| 2-21-606 | N-Biph | N-Biph | b3 | b3 | b9 |
| 2-21-607 | N-Biph | N-Biph | H | H | b10 |
| 2-21-608 | N-Biph | N-Biph | t-bu | t-bu | b10 |
| 2-21-609 | N-Biph | N-Biph | methyl | methyl | b10 |
| 2-21-610 | N-Biph | N-Biph | b1 | b1 | b10 |
| 2-21-611 | N-Biph | N-Biph | b2 | b2 | b10 |
| 2-21-612 | N-Biph | N-Biph | b3 | b3 | b10 |
| 2-21-613 | N-Biph | N-Biph | H | H | H |
| 2-21-614 | N-Biph | N-Biph | t-bu | t-bu | H |
| 2-21-615 | N-Biph | N-Biph | methyl | methyl | H |
| 2-21-616 | N-Biph | N-Biph | b1 | b1 | H |
| 2-21-617 | N-Biph | N-Biph | b2 | b2 | H |
| 2-21-618 | N-Biph | N-Biph | b3 | b3 | H |
| 2-21-619 | N-Biph | N-Biph | H | H | H |
| 2-21-620 | N-Biph | N-Biph | t-bu | t-bu | H |
| 2-21-621 | N-Biph | N-Biph | methyl | methyl | H |
| 2-21-622 | N-Biph | N-Biph | b1 | b1 | H |
| 2-21-623 | N-Biph | N-Biph | b2 | b2 | H |
| 2-21-624 | N-Biph | N-Biph | b3 | b3 | H |
| 2-21-625 | N-Biph | N-Biph | H | H | H |
| 2-21-626 | N-Biph | N-Biph | t-bu | t-bu | H |
| 2-21-627 | N-Biph | N-Biph | methyl | methyl | H |
| 2-21-628 | N-Biph | N-Biph | b1 | b1 | H |
| 2-21-629 | N-Biph | N-Biph | b2 | b2 | H |
| 2-21-630 | N-Biph | N-Biph | b3 | b3 | H |
| 2-21-631 | N-Biph | N-Biph | H | H | H |
| 2-21-632 | N-Biph | N-Biph | t-bu | t-bu | H |
| 2-21-633 | N-Biph | N-Biph | methyl | methyl | H |
| 2-21-634 | N-Biph | N-Biph | b1 | b1 | H |
| 2-21-635 | N-Biph | N-Biph | b2 | b2 | H |
| 2-21-636 | N-Biph | N-Biph | b3 | b3 | H |
| 2-21-637 | N-Biph | N-Biph | H | H | H |
| 2-21-638 | N-Biph | N-Biph | t-bu | t-bu | H |
| 2-21-639 | N-Biph | N-Biph | methyl | methyl | H |
| 2-21-640 | N-Biph | N-Biph | b1 | b1 | H |
| 2-21-641 | N-Biph | N-Biph | b2 | b2 | H |
| 2-21-642 | N-Biph | N-Biph | b3 | b3 | H |
| 2-21-643 | N-Biph | N-Biph | H | H | H |
| 2-21-644 | N-Biph | N-Biph | t-bu | t-bu | H |
| 2-21-645 | N-Biph | N-Biph | methyl | methyl | H |
| 2-21-646 | N-Biph | N-Biph | b1 | b1 | H |
| 2-21-647 | N-Biph | N-Biph | b2 | b2 | H |
| 2-21-648 | N-Biph | N-Biph | b3 | b3 | H |
| 2-21-649 | N-Biph | N-Biph | H | H | H |
| 2-21-650 | N-Biph | N-Biph | t-bu | t-bu | H |
| 2-21-651 | N-Biph | N-Biph | methyl | methyl | H |
| 2-21-652 | N-Biph | N-Biph | b1 | b1 | H |
| 2-21-653 | N-Biph | N-Biph | b2 | b2 | H |
| 2-21-654 | N-Biph | N-Biph | b3 | b3 | H |
| 2-21-655 | N-Biph | N-Biph | H | H | H |
| 2-21-656 | N-Biph | N-Biph | t-bu | t-bu | H |
| 2-21-657 | N-Biph | N-Biph | methyl | methyl | H |
| 2-21-658 | N-Biph | N-Biph | b1 | b1 | H |
| 2-21-659 | N-Biph | N-Biph | b2 | b2 | H |
| 2-21-660 | N-Biph | N-Biph | b3 | b3 | H |
| 2-21-661 | N-Biph | N-Biph | H | H | H |
| 2-21-662 | N-Biph | N-Biph | t-bu | t-bu | H |
| 2-21-663 | N-Biph | N-Biph | methyl | methyl | H |
| 2-21-664 | N-Biph | N-Biph | b1 | b1 | H |
| 2-21-665 | N-Biph | N-Biph | b2 | b2 | H |
| 2-21-666 | N-Biph | N-Biph | b3 | b3 | H |
| 2-21-667 | N-Biph | N-Biph | H | H | H |
| 2-21-668 | N-Biph | N-Biph | t-bu | t-bu | H |
| 2-21-669 | N-Biph | N-Biph | methyl | methyl | H |
| 2-21-670 | N-Biph | N-Biph | b1 | b1 | H |
| 2-21-671 | N-Biph | N-Biph | b2 | b2 | H |
| 2-21-672 | N-Biph | N-Biph | b3 | b3 | H |
| 2-21-673 | N-Biph | N-Biph | H | H | H |
| 2-21-674 | N-Biph | N-Biph | t-bu | t-bu | H |
| 2-21-675 | N-Biph | N-Biph | methyl | methyl | H |
| 2-21-676 | N-Biph | N-Biph | b1 | b1 | H |
| 2-21-677 | N-Biph | N-Biph | b2 | b2 | H |
| 2-21-678 | N-Biph | N-Biph | b3 | b3 | H |
| 2-21-679 | N-Biph | N-Biph | H | H | H |
| 2-21-680 | N-Biph | N-Biph | t-bu | t-bu | H |
| 2-21-681 | N-Biph | N-Biph | methyl | methyl | H |
| 2-21-682 | N-Biph | N-Biph | b1 | b1 | H |
| 2-21-683 | N-Biph | N-Biph | b2 | b2 | H |
| 2-21-684 | N-Biph | N-Biph | b3 | b3 | H |
| 2-21-685 | N-Biph | N-Biph | H | H | H |
| 2-21-686 | N-Biph | N-Biph | t-bu | t-bu | H |
| 2-21-687 | N-Biph | N-Biph | methyl | methyl | H |
| 2-21-688 | N-Biph | N-Biph | b1 | b1 | H |
| 2-21-689 | N-Biph | N-Biph | b2 | b2 | H |
| 2-21-690 | N-Biph | N-Biph | b3 | b3 | H |
| 2-21-691 | N-Biph | N-Biph | H | H | H |
| 2-21-692 | N-Biph | N-Biph | t-bu | t-bu | H |
| 2-21-693 | N-Biph | N-Biph | methyl | methyl | H |
| 2-21-694 | N-Biph | N-Biph | b1 | b1 | H |
| 2-21-695 | N-Biph | N-Biph | b2 | b2 | H |
| 2-21-696 | N-Biph | N-Biph | b3 | b3 | H |
| 2-21-697 | N-Biph | N-Biph | H | H | H |
| 2-21-698 | N-Biph | N-Biph | t-bu | t-bu | H |
| 2-21-699 | N-Biph | N-Biph | methyl | methyl | H |
| 2-21-700 | N-Biph | N-Biph | b1 | b1 | H |
| 2-21-701 | N-Biph | N-Biph | b2 | b2 | H |
| 2-21-702 | N-Biph | N-Biph | b3 | b3 | H |
| 2-21-703 | N-Biph | N-Biph | H | H | H |
| 2-21-704 | N-Biph | N-Biph | t-bu | t-bu | H |
| 2-21-705 | N-Biph | N-Biph | methyl | methyl | H |
| 2-21-706 | N-Biph | N-Biph | b1 | b1 | H |
| 2-21-707 | N-Biph | N-Biph | b2 | b2 | H |
| 2-21-708 | N-Biph | N-Biph | b3 | b3 | H |
| 2-21-709 | N-Biph | N-Biph | H | H | a1 |
| 2-21-710 | N-Biph | N-Biph | t-bu | t-bu | a1 |
| 2-21-711 | N-Biph | N-Biph | methyl | methyl | a1 |
| 2-21-712 | N-Biph | N-Biph | b1 | b1 | a1 |
| 2-21-713 | N-Biph | N-Biph | b2 | b2 | a1 |
| 2-21-714 | N-Biph | N-Biph | b3 | b3 | a1 |
| 2-21-715 | N-Biph | N-Biph | H | H | a2 |
| 2-21-716 | N-Biph | N-Biph | t-bu | t-bu | a2 |
| 2-21-717 | N-Biph | N-Biph | methyl | methyl | a2 |
| 2-21-718 | N-Biph | N-Biph | b1 | b1 | a2 |
| 2-21-719 | N-Biph | N-Biph | b2 | b2 | a2 |
| 2-21-720 | N-Biph | N-Biph | b3 | b3 | a2 |
| 2-21-721 | N-Biph | N-Biph | H | H | a3 |
| 2-21-722 | N-Biph | N-Biph | t-bu | t-bu | a3 |
| 2-21-723 | N-Biph | N-Biph | methyl | methyl | a3 |
| 2-21-724 | N-Biph | N-Biph | b1 | b1 | a3 |
| 2-21-725 | N-Biph | N-Biph | b2 | b2 | a3 |
| 2-21-726 | N-Biph | N-Biph | b3 | b3 | a3 |
| 2-21-727 | N-Biph | N-Biph | H | H | b1 |
| 2-21-728 | N-Biph | N-Biph | t-bu | t-bu | b1 |
| 2-21-729 | N-Biph | N-Biph | methyl | methyl | b1 |
| 2-21-730 | N-Biph | N-Biph | b1 | b1 | b1 |
| 2-21-731 | N-Biph | N-Biph | b2 | b2 | b1 |
| 2-21-732 | N-Biph | N-Biph | b3 | b3 | b1 |
| 2-21-733 | N-Biph | N-Biph | H | H | b2 |
| 2-21-734 | N-Biph | N-Biph | t-bu | t-bu | b2 |
| 2-21-735 | N-Biph | N-Biph | methyl | methyl | b2 |
| 2-21-736 | N-Biph | N-Biph | b1 | b1 | b2 |
| 2-21-737 | N-Biph | N-Biph | b2 | b2 | b2 |
| 2-21-738 | N-Biph | N-Biph | b3 | b3 | b2 |
| 2-21-739 | N-Biph | N-Biph | H | H | b3 |
| 2-21-740 | N-Biph | N-Biph | t-bu | t-bu | b3 |
| 2-21-741 | N-Biph | N-Biph | methyl | methyl | b3 |
| 2-21-742 | N-Biph | N-Biph | b1 | b1 | b3 |
| 2-21-743 | N-Biph | N-Biph | b2 | b2 | b3 |
| 2-21-744 | N-Biph | N-Biph | b3 | b3 | b3 |
| 2-21-745 | N-Biph | N-Biph | H | H | b5 |
| 2-21-746 | N-Biph | N-Biph | t-bu | t-bu | b5 |
| 2-21-747 | N-Biph | N-Biph | methyl | methyl | b5 |
| 2-21-748 | N-Biph | N-Biph | b1 | b1 | b5 |
| 2-21-749 | N-Biph | N-Biph | b2 | b2 | b5 |
| 2-21-750 | N-Biph | N-Biph | b3 | b3 | b5 |
| 2-21-751 | N-Biph | N-Biph | H | H | b6 |
| 2-21-752 | N-Biph | N-Biph | t-bu | t-bu | b6 |
| 2-21-753 | N-Biph | N-Biph | methyl | methyl | b6 |
| 2-21-754 | N-Biph | N-Biph | b1 | b1 | b6 |
| 2-21-755 | N-Biph | N-Biph | b2 | b2 | b6 |
| 2-21-756 | N-Biph | N-Biph | b3 | b3 | b6 |
| 2-21-757 | N-Biph | N-Biph | H | H | b7 |
| 2-21-758 | N-Biph | N-Biph | t-bu | t-bu | b7 |
| 2-21-759 | N-Biph | N-Biph | methyl | methyl | b7 |
| 2-21-760 | N-Biph | N-Biph | b1 | b1 | b7 |
| 2-21-761 | N-Biph | N-Biph | b2 | b2 | b7 |
| 2-21-762 | N-Biph | N-Biph | b3 | b3 | b7 |
| 2-21-763 | N—Ph | O | H | H | H |
| 2-21-764 | N—Ph | O | t-bu | t-bu | H |
| 2-21-765 | N—Ph | O | methyl | methyl | H |
| 2-21-766 | N—Ph | O | t-bu | t-bu | t-bu |
| 2-21-767 | N—Ph | O | methyl | methyl | methyl |
| 2-21-768 | N—Ph | O | b1 | b1 | H |
| 2-21-769 | N—Ph | O | b2 | b2 | H |
| 2-21-770 | N—Ph | O | b3 | b3 | H |
| 2-21-771 | N—Ph | O | H | H | a1 |
| 2-21-772 | N—Ph | O | t-bu | t-bu | a1 |
| 2-21-773 | N—Ph | O | methyl | methyl | a1 |
| 2-21-774 | N—Ph | O | b1 | b1 | a1 |
| 2-21-775 | N—Ph | O | b2 | b2 | a1 |
| 2-21-776 | N—Ph | O | b3 | b3 | a1 |
| 2-21-777 | N—Ph | O | H | H | a2 |
| 2-21-778 | N—Ph | O | t-bu | t-bu | a2 |
| 2-21-779 | N—Ph | O | methyl | methyl | a2 |
| 2-21-780 | N—Ph | O | b1 | b1 | a2 |
| 2-21-781 | N—Ph | O | b2 | b2 | a2 |
| 2-21-782 | N—Ph | O | b3 | b3 | a2 |
| 2-21-783 | N—Ph | O | H | H | a3 |
| 2-21-784 | N—Ph | O | t-bu | t-bu | a3 |
| 2-21-785 | N—Ph | O | methyl | methyl | a3 |
| 2-21-786 | N—Ph | O | b1 | b1 | a3 |
| 2-21-787 | N—Ph | O | b2 | b2 | a3 |
| 2-21-788 | N—Ph | O | b3 | b3 | a3 |
| 2-21-789 | N—Ph | O | H | H | a4 |
| 2-21-790 | N—Ph | O | t-bu | t-bu | a4 |
| 2-21-791 | N—Ph | O | methyl | methyl | a4 |
| 2-21-792 | N—Ph | O | b1 | b1 | a4 |
| 2-21-793 | N—Ph | O | b2 | b2 | a4 |
| 2-21-794 | N—Ph | O | b3 | b3 | a4 |
| 2-21-795 | N—Ph | O | H | H | a5 |
| 2-21-796 | N—Ph | O | t-bu | t-bu | a5 |
| 2-21-797 | N—Ph | O | methyl | methyl | a5 |
| 2-21-798 | N—Ph | O | b1 | b1 | a5 |
| 2-21-799 | N—Ph | O | b2 | b2 | a5 |
| 2-21-800 | N—Ph | O | b3 | b3 | a5 |
| 2-21-801 | N—Ph | O | H | H | a6 |
| 2-21-802 | N—Ph | O | t-bu | t-bu | a6 |
| 2-21-803 | N—Ph | O | methyl | methyl | a6 |
| 2-21-804 | N—Ph | O | b1 | b1 | a6 |
| 2-21-805 | N—Ph | O | b2 | b2 | a6 |
| 2-21-806 | N—Ph | O | b3 | b3 | a6 |
| 2-21-807 | N—Ph | O | H | H | b1 |
| 2-21-808 | N—Ph | O | t-bu | t-bu | b1 |
| 2-21-809 | N—Ph | O | methyl | methyl | b1 |
| 2-21-810 | N—Ph | O | b1 | b1 | b1 |
| 2-21-811 | N—Ph | O | b2 | b2 | b1 |
| 2-21-812 | N—Ph | O | b3 | b3 | b1 |
| 2-21-813 | N—Ph | O | H | H | b2 |
| 2-21-814 | N—Ph | O | t-bu | t-bu | b2 |
| 2-21-815 | N—Ph | O | methyl | methyl | b2 |
| 2-21-816 | N—Ph | O | b1 | b1 | b2 |
| 2-21-817 | N—Ph | O | b2 | b2 | b2 |
| 2-21-818 | N—Ph | O | b3 | b3 | b2 |
| 2-21-819 | N—Ph | O | H | H | b3 |
| 2-21-820 | N—Ph | O | t-bu | t-bu | b3 |
| 2-21-821 | N—Ph | O | methyl | methyl | b3 |
| 2-21-822 | N—Ph | O | b1 | b1 | b3 |
| 2-21-823 | N—Ph | O | b2 | b2 | b3 |
| 2-21-824 | N—Ph | O | b3 | b3 | b3 |
| 2-21-825 | N—Ph | O | H | H | b4 |
| 2-21-826 | N—Ph | O | t-bu | t-bu | b4 |
| 2-21-827 | N—Ph | O | methyl | methyl | b4 |
| 2-21-828 | N—Ph | O | b1 | b1 | b4 |
| 2-21-829 | N—Ph | O | b2 | b2 | b4 |
| 2-21-830 | N—Ph | O | b3 | b3 | b4 |
| 2-21-831 | N—Ph | O | H | H | b5 |
| 2-21-832 | N—Ph | O | t-bu | t-bu | b5 |
| 2-21-833 | N—Ph | O | methyl | methyl | b5 |
| 2-21-834 | N—Ph | O | b1 | b1 | b5 |
| 2-21-835 | N—Ph | O | b2 | b2 | b5 |
| 2-21-836 | N—Ph | O | b3 | b3 | b5 |
| 2-21-837 | N—Ph | O | H | H | b6 |
| 2-21-838 | N—Ph | O | t-bu | t-bu | b6 |
| 2-21-839 | N—Ph | O | methyl | methyl | b6 |
| 2-21-840 | N—Ph | O | b1 | b1 | b6 |
| 2-21-841 | N—Ph | O | b2 | b2 | b6 |
| 2-21-842 | N—Ph | O | b3 | b3 | b6 |
| 2-21-843 | N—Ph | O | H | H | b7 |
| 2-21-844 | N—Ph | O | t-bu | t-bu | b7 |
| 2-21-845 | N—Ph | O | methyl | methyl | b7 |
| 2-21-846 | N—Ph | O | b1 | b1 | b7 |
| 2-21-847 | N—Ph | O | b2 | b2 | b7 |
| 2-21-848 | N—Ph | O | b3 | b3 | b7 |
| 2-21-849 | N—Ph | O | H | H | b8 |
| 2-21-850 | N—Ph | O | t-bu | t-bu | b8 |
| 2-21-851 | N—Ph | O | methyl | methyl | b8 |
| 2-21-852 | N—Ph | O | b1 | b1 | b8 |
| 2-21-853 | N—Ph | O | b2 | b2 | b8 |
| 2-21-854 | N—Ph | O | b3 | b3 | b8 |
| 2-21-855 | N—Ph | O | H | H | b9 |
| 2-21-856 | N—Ph | O | t-bu | t-bu | b9 |
| 2-21-857 | N—Ph | O | methyl | methyl | b9 |
| 2-21-858 | N—Ph | O | b1 | b1 | b9 |
| 2-21-859 | N—Ph | O | b2 | b2 | b9 |
| 2-21-860 | N—Ph | O | b3 | b3 | b9 |
| 2-21-861 | N—Ph | O | H | H | b10 |
| 2-21-862 | N—Ph | O | t-bu | t-bu | b10 |
| 2-21-863 | N—Ph | O | methyl | methyl | b10 |
| 2-21-864 | N—Ph | O | b1 | b1 | b10 |
| 2-21-865 | N—Ph | O | b2 | b2 | b10 |
| 2-21-866 | N—Ph | O | b3 | b3 | b10 |
| 2-21-867 | N—Ph | O | H | H | H |
| 2-21-868 | N—Ph | O | t-bu | t-bu | H |
| 2-21-869 | N—Ph | O | methyl | methyl | H |
| 2-21-870 | N—Ph | O | b1 | b1 | H |
| 2-21-871 | N—Ph | O | b2 | b2 | H |
| 2-21-872 | N—Ph | O | b3 | b3 | H |
| 2-21-873 | N—Ph | O | H | H | H |
| 2-21-874 | N—Ph | O | t-bu | t-bu | H |
| 2-21-875 | N—Ph | O | methyl | methyl | H |
| 2-21-876 | N—Ph | O | b1 | b1 | H |
| 2-21-877 | N—Ph | O | b2 | b2 | H |
| 2-21-878 | N—Ph | O | b3 | b3 | H |
| 2-21-879 | N—Ph | O | H | H | H |
| 2-21-880 | N—Ph | O | t-bu | t-bu | H |
| 2-21-881 | N—Ph | O | methyl | methyl | H |
| 2-21-882 | N—Ph | O | b1 | b1 | H |
| 2-21-883 | N—Ph | O | b2 | b2 | H |
| 2-21-884 | N—Ph | O | b3 | b3 | H |
| 2-21-885 | N—Ph | O | H | H | H |
| 2-21-886 | N—Ph | O | t-bu | t-bu | H |
| 2-21-887 | N—Ph | O | methyl | methyl | H |
| 2-21-888 | N—Ph | O | b1 | b1 | H |
| 2-21-889 | N—Ph | O | b2 | b2 | H |
| 2-21-890 | N—Ph | O | b3 | b3 | H |
| 2-21-891 | N—Ph | O | H | H | H |
| 2-21-892 | N—Ph | O | t-bu | t-bu | H |
| 2-21-893 | N—Ph | O | methyl | methyl | H |
| 2-21-894 | N—Ph | O | b1 | b1 | H |
| 2-21-895 | N—Ph | O | b2 | b2 | H |
| 2-21-896 | N—Ph | O | b3 | b3 | H |
| 2-21-897 | N—Ph | O | H | H | H |
| 2-21-898 | N—Ph | O | t-bu | t-bu | H |
| 2-21-899 | N—Ph | O | methyl | methyl | H |
| 2-21-900 | N—Ph | O | b1 | b1 | H |
| 2-21-901 | N—Ph | O | b2 | b2 | H |
| 2-21-902 | N—Ph | O | b3 | b3 | H |
| 2-21-903 | N—Ph | O | H | H | H |
| 2-21-904 | N—Ph | O | t-bu | t-bu | H |
| 2-21-905 | N—Ph | O | methyl | methyl | H |
| 2-21-906 | N—Ph | O | b1 | b1 | H |
| 2-21-907 | N—Ph | O | b2 | b2 | H |
| 2-21-908 | N—Ph | O | b3 | b3 | H |
| 2-21-909 | N—Ph | O | H | H | H |
| 2-21-910 | N—Ph | O | t-bu | t-bu | H |
| 2-21-911 | N—Ph | O | methyl | methyl | H |
| 2-21-912 | N—Ph | O | b1 | b1 | H |
| 2-21-913 | N—Ph | O | b2 | b2 | H |
| 2-21-914 | N—Ph | O | b3 | b3 | H |
| 2-21-915 | N—Ph | O | H | H | H |
| 2-21-916 | N—Ph | O | t-bu | t-bu | H |
| 2-21-917 | N—Ph | O | methyl | methyl | H |
| 2-21-918 | N—Ph | O | b1 | b1 | H |
| 2-21-919 | N—Ph | O | b2 | b2 | H |
| 2-21-920 | N—Ph | O | b3 | b3 | H |
| 2-21-921 | N—Ph | O | H | H | H |
| 2-21-922 | N—Ph | O | t-bu | t-bu | H |
| 2-21-923 | N—Ph | O | methyl | methyl | H |
| 2-21-924 | N—Ph | O | b1 | b1 | H |
| 2-21-925 | N—Ph | O | b2 | b2 | H |
| 2-21-926 | N—Ph | O | b3 | b3 | H |
| 2-21-927 | N—Ph | O | H | H | H |
| 2-21-928 | N—Ph | O | t-bu | t-bu | H |
| 2-21-929 | N—Ph | O | methyl | methyl | H |
| 2-21-930 | N—Ph | O | b1 | b1 | H |
| 2-21-931 | N—Ph | O | b2 | b2 | H |
| 2-21-932 | N—Ph | O | b3 | b3 | H |
| 2-21-933 | N—Ph | O | H | H | H |
| 2-21-934 | N—Ph | O | t-bu | t-bu | H |
| 2-21-935 | N—Ph | O | methyl | methyl | H |
| 2-21-936 | N—Ph | O | b1 | b1 | H |
| 2-21-937 | N—Ph | O | b2 | b2 | H |
| 2-21-938 | N—Ph | O | b3 | b3 | H |
| 2-21-939 | N—Ph | O | H | H | H |
| 2-21-940 | N—Ph | O | t-bu | t-bu | H |
| 2-21-941 | N—Ph | O | methyl | methyl | H |
| 2-21-942 | N—Ph | O | b1 | b1 | H |
| 2-21-943 | N—Ph | O | b2 | b2 | H |
| 2-21-944 | N—Ph | O | b3 | b3 | H |
| 2-21-945 | N—Ph | O | H | H | H |
| 2-21-946 | N—Ph | O | t-bu | t-bu | H |
| 2-21-947 | N—Ph | O | methyl | methyl | H |
| 2-21-948 | N—Ph | O | b1 | b1 | H |
| 2-21-949 | N—Ph | O | b2 | b2 | H |
| 2-21-950 | N—Ph | O | b3 | b3 | H |
| 2-21-951 | N—Ph | O | H | H | H |
| 2-21-952 | N—Ph | O | t-bu | t-bu | H |
| 2-21-953 | N—Ph | O | methyl | methyl | H |
| 2-21-954 | N—Ph | O | b1 | b1 | H |
| 2-21-955 | N—Ph | O | b2 | b2 | H |
| 2-21-956 | N—Ph | O | b3 | b3 | H |
| 2-21-957 | N—Ph | O | H | H | H |
| 2-21-958 | N—Ph | O | t-bu | t-bu | H |
| 2-21-959 | N—Ph | O | methyl | methyl | H |
| 2-21-960 | N—Ph | O | b1 | b1 | H |
| 2-21-961 | N—Ph | O | b2 | b2 | H |
| 2-21-962 | N—Ph | O | b3 | b3 | H |
| 2-21-963 | N—Ph | O | H | H | a1 |
| 2-21-964 | N—Ph | O | t-bu | t-bu | a1 |
| 2-21-965 | N—Ph | O | methyl | methyl | a1 |
| 2-21-966 | N—Ph | O | b1 | b1 | a1 |
| 2-21-967 | N—Ph | O | b2 | b2 | a1 |
| 2-21-968 | N—Ph | O | b3 | b3 | a1 |
| 2-21-969 | N—Ph | O | H | H | a2 |
| 2-21-970 | N—Ph | O | t-bu | t-bu | a2 |
| 2-21-971 | N—Ph | O | methyl | methyl | a2 |
| 2-21-972 | N—Ph | O | b1 | b1 | a2 |
| 2-21-973 | N—Ph | O | b2 | b2 | a2 |
| 2-21-974 | N—Ph | O | b3 | b3 | a2 |
| 2-21-975 | N—Ph | O | H | H | a3 |
| 2-21-976 | N—Ph | O | t-bu | t-bu | a3 |
| 2-21-977 | N—Ph | O | methyl | methyl | a3 |
| 2-21-978 | N—Ph | O | b1 | b1 | a3 |
| 2-21-979 | N—Ph | O | b2 | b2 | a3 |
| 2-21-980 | N—Ph | O | b3 | b3 | a3 |
| 2-21-981 | N—Ph | O | H | H | b1 |
| 2-21-982 | N—Ph | O | t-bu | t-bu | b1 |
| 2-21-983 | N—Ph | O | methyl | methyl | b1 |
| 2-21-984 | N—Ph | O | b1 | b1 | b1 |
| 2-21-985 | N—Ph | O | b2 | b2 | b1 |
| 2-21-986 | N—Ph | O | b3 | b3 | b1 |
| 2-21-987 | N—Ph | O | H | H | b2 |
| 2-21-988 | N—Ph | O | t-bu | t-bu | b2 |
| 2-21-989 | N—Ph | O | methyl | methyl | b2 |
| 2-21-990 | N—Ph | O | b1 | b1 | b2 |
| 2-21-991 | N—Ph | O | b2 | b2 | b2 |
| 2-21-992 | N—Ph | O | b3 | b3 | b2 |
| 2-21-993 | N—Ph | O | H | H | b3 |
| 2-21-994 | N—Ph | O | t-bu | t-bu | b3 |
| 2-21-995 | N—Ph | O | methyl | methyl | b3 |
| 2-21-996 | N—Ph | O | b1 | b1 | b3 |
| 2-21-997 | N—Ph | O | b2 | b2 | b3 |
| 2-21-998 | N—Ph | O | b3 | b3 | b3 |
| 2-21-999 | N—Ph | O | H | H | b5 |
| 2-21-1000 | N—Ph | O | t-bu | t-bu | b5 |
| 2-21-1001 | N—Ph | O | methyl | methyl | b5 |
| 2-21-1002 | N—Ph | O | b1 | b1 | b5 |
| 2-21-1003 | N—Ph | O | b2 | b2 | b5 |
| 2-21-1004 | N—Ph | O | b3 | b3 | b5 |
| 2-21-1005 | N—Ph | O | H | H | b6 |
| 2-21-1006 | N—Ph | O | t-bu | t-bu | b6 |
| 2-21-1007 | N—Ph | O | methyl | methyl | b6 |
| 2-21-1008 | N—Ph | O | b1 | b1 | b6 |
| 2-21-1009 | N—Ph | O | b2 | b2 | b6 |
| 2-21-1010 | N—Ph | O | b3 | b3 | b6 |
| 2-21-1011 | N—Ph | O | H | H | b7 |
| 2-21-1012 | N—Ph | O | t-bu | t-bu | b7 |
| 2-21-1013 | N—Ph | O | methyl | methyl | b7 |
| 2-21-1014 | N—Ph | O | b1 | b1 | b7 |
| 2-21-1015 | N—Ph | O | b2 | b2 | b7 |
| 2-21-1016 | N—Ph | O | b3 | b3 | b7 |
| 2-21-1017 | N—Ph | S | H | H | H |
| 2-21-1018 | N—Ph | S | t-bu | t-bu | H |
| 2-21-1019 | N—Ph | S | methyl | methyl | H |
| 2-21-1020 | N—Ph | S | t-bu | t-bu | t-bu |
| 2-21-1021 | N—Ph | S | methyl | methyl | methyl |
| 2-21-1022 | N—Ph | S | b1 | b1 | H |
| 2-21-1023 | N—Ph | S | b2 | b2 | H |
| 2-21-1024 | N—Ph | S | b3 | b3 | H |
| 2-21-1025 | N—Ph | S | H | H | a1 |
| 2-21-1026 | N—Ph | S | t-bu | t-bu | a1 |
| 2-21-1027 | N—Ph | S | methyl | methyl | a1 |
| 2-21-1028 | N—Ph | S | b1 | b1 | a1 |
| 2-21-1029 | N—Ph | S | b2 | b2 | a1 |
| 2-21-1030 | N—Ph | S | b3 | b3 | a1 |
| 2-21-1031 | N—Ph | S | H | H | a2 |
| 2-21-1032 | N—Ph | S | t-bu | t-bu | a2 |
| 2-21-1033 | N—Ph | S | methyl | methyl | a2 |
| 2-21-1034 | N—Ph | S | b1 | b1 | a2 |
| 2-21-1035 | N—Ph | S | b2 | b2 | a2 |
| 2-21-1036 | N—Ph | S | b3 | b3 | a2 |
| 2-21-1037 | N—Ph | S | H | H | a3 |
| 2-21-1038 | N—Ph | S | t-bu | t-bu | a3 |
| 2-21-1039 | N—Ph | S | methyl | methyl | a3 |
| 2-21-1040 | N—Ph | S | b1 | b1 | a3 |
| 2-21-1041 | N—Ph | S | b2 | b2 | a3 |
| 2-21-1042 | N—Ph | S | b3 | b3 | a3 |
| 2-21-1043 | N—Ph | S | H | H | a4 |
| 2-21-1044 | N—Ph | S | t-bu | t-bu | a4 |
| 2-21-1045 | N—Ph | S | methyl | methyl | a4 |
| 2-21-1046 | N—Ph | S | b1 | b1 | a4 |
| 2-21-1047 | N—Ph | S | b2 | b2 | a4 |
| 2-21-1048 | N—Ph | S | b3 | b3 | a4 |
| 2-21-1049 | N—Ph | S | H | H | a5 |
| 2-21-1050 | N—Ph | S | t-bu | t-bu | a5 |
| 2-21-1051 | N—Ph | S | methyl | methyl | a5 |
| 2-21-1052 | N—Ph | S | b1 | b1 | a5 |
| 2-21-1053 | N—Ph | S | b2 | b2 | a5 |
| 2-21-1054 | N—Ph | S | b3 | b3 | a5 |
| 2-21-1055 | N—Ph | S | H | H | a6 |
| 2-21-1056 | N—Ph | S | t-bu | t-bu | a6 |
| 2-21-1057 | N—Ph | S | methyl | methyl | a6 |
| 2-21-1058 | N—Ph | S | b1 | b1 | a6 |
| 2-21-1059 | N—Ph | S | b2 | b2 | a6 |
| 2-21-1060 | N—Ph | S | b3 | b3 | a6 |
| 2-21-1061 | N—Ph | S | H | H | b1 |
| 2-21-1062 | N—Ph | S | t-bu | t-bu | b1 |
| 2-21-1063 | N—Ph | S | methyl | methyl | b1 |
| 2-21-1064 | N—Ph | S | b1 | b1 | b1 |
| 2-21-1065 | N—Ph | S | b2 | b2 | b1 |
| 2-21-1066 | N—Ph | S | b3 | b3 | b1 |
| 2-21-1067 | N—Ph | S | H | H | b2 |
| 2-21-1068 | N—Ph | S | t-bu | t-bu | b2 |
| 2-21-1069 | N—Ph | S | methyl | methyl | b2 |
| 2-21-1070 | N—Ph | S | b1 | b1 | b2 |
| 2-21-1071 | N—Ph | S | b2 | b2 | b2 |
| 2-21-1072 | N—Ph | S | b3 | b3 | b2 |
| 2-21-1073 | N—Ph | S | H | H | b3 |
| 2-21-1074 | N—Ph | S | t-bu | t-bu | b3 |
| 2-21-1075 | N—Ph | S | methyl | methyl | b3 |
| 2-21-1076 | N—Ph | S | b1 | b1 | b3 |
| 2-21-1077 | N—Ph | S | b2 | b2 | b3 |
| 2-21-1078 | N—Ph | S | b3 | b3 | b3 |
| 2-21-1079 | N—Ph | S | H | H | b4 |
| 2-21-1080 | N—Ph | S | t-bu | t-bu | b4 |
| 2-21-1081 | N—Ph | S | methyl | methyl | b4 |
| 2-21-1082 | N—Ph | S | b1 | b1 | b4 |
| 2-21-1083 | N—Ph | S | b2 | b2 | b4 |
| 2-21-1084 | N—Ph | S | b3 | b3 | b4 |
| 2-21-1085 | N—Ph | S | H | H | b5 |
| 2-21-1086 | N—Ph | S | t-bu | t-bu | b5 |
| 2-21-1087 | N—Ph | S | methyl | methyl | b5 |
| 2-21-1088 | N—Ph | S | b1 | b1 | b5 |
| 2-21-1089 | N—Ph | S | b2 | b2 | b5 |
| 2-21-1090 | N—Ph | S | b3 | b3 | b5 |
| 2-21-1091 | N—Ph | S | H | H | b6 |
| 2-21-1092 | N—Ph | S | t-bu | t-bu | b6 |
| 2-21-1093 | N—Ph | S | methyl | methyl | b6 |
| 2-21-1094 | N—Ph | S | b1 | b1 | b6 |
| 2-21-1095 | N—Ph | S | b2 | b2 | b6 |
| 2-21-1096 | N—Ph | S | b3 | b3 | b6 |
| 2-21-1097 | N—Ph | S | H | H | b7 |
| 2-21-1098 | N—Ph | S | t-bu | t-bu | b7 |
| 2-21-1099 | N—Ph | S | methyl | methyl | b7 |
| 2-21-1100 | N—Ph | S | b1 | b1 | b7 |
| 2-21-1101 | N—Ph | S | b2 | b2 | b7 |
| 2-21-1102 | N—Ph | S | b3 | b3 | b7 |
| 2-21-1103 | N—Ph | S | H | H | b8 |
| 2-21-1104 | N—Ph | S | t-bu | t-bu | b8 |
| 2-21-1105 | N—Ph | S | methyl | methyl | b8 |
| 2-21-1106 | N—Ph | S | b1 | b1 | b8 |
| 2-21-1107 | N—Ph | S | b2 | b2 | b8 |
| 2-21-1108 | N—Ph | S | b3 | b3 | b8 |
| 2-21-1109 | N—Ph | S | H | H | b9 |
| 2-21-1110 | N—Ph | S | t-bu | t-bu | b9 |
| 2-21-1111 | N—Ph | S | methyl | methyl | b9 |
| 2-21-1112 | N—Ph | S | b1 | b1 | b9 |
| 2-21-1113 | N—Ph | S | b2 | b2 | b9 |
| 2-21-1114 | N—Ph | S | b3 | b3 | b9 |
| 2-21-1115 | N—Ph | S | H | H | b10 |
| 2-21-1116 | N—Ph | S | t-bu | t-bu | b10 |
| 2-21-1117 | N—Ph | S | methyl | methyl | b10 |
| 2-21-1118 | N—Ph | S | b1 | b1 | b10 |
| 2-21-1119 | N—Ph | S | b2 | b2 | b10 |
| 2-21-1120 | N—Ph | S | b3 | b3 | b10 |
| 2-21-1121 | N—Ph | S | H | H | H |
| 2-21-1122 | N—Ph | S | t-bu | t-bu | H |
| 2-21-1123 | N—Ph | S | methyl | methyl | H |
| 2-21-1124 | N—Ph | S | b1 | b1 | H |
| 2-21-1125 | N—Ph | S | b2 | b2 | H |
| 2-21-1126 | N—Ph | S | b3 | b3 | H |
| 2-21-1127 | N—Ph | S | H | H | H |
| 2-21-1128 | N—Ph | S | t-bu | t-bu | H |
| 2-21-1129 | N—Ph | S | methyl | methyl | H |
| 2-21-1130 | N—Ph | S | b1 | b1 | H |
| 2-21-1131 | N—Ph | S | b2 | b2 | H |
| 2-21-1132 | N—Ph | S | b3 | b3 | H |
| 2-21-1133 | N—Ph | S | H | H | H |
| 2-21-1134 | N—Ph | S | t-bu | t-bu | H |
| 2-21-1135 | N—Ph | S | methyl | methyl | H |
| 2-21-1136 | N—Ph | S | b1 | b1 | H |
| 2-21-1137 | N—Ph | S | b2 | b2 | H |
| 2-21-1138 | N—Ph | S | b3 | b3 | H |
| 2-21-1139 | N—Ph | S | H | H | H |
| 2-21-1140 | N—Ph | S | t-bu | t-bu | H |
| 2-21-1141 | N—Ph | S | methyl | methyl | H |
| 2-21-1142 | N—Ph | S | b1 | b1 | H |
| 2-21-1143 | N—Ph | S | b2 | b2 | H |
| 2-21-1144 | N—Ph | S | b3 | b3 | H |
| 2-21-1145 | N—Ph | S | H | H | H |
| 2-21-1146 | N—Ph | S | t-bu | t-bu | H |
| 2-21-1147 | N—Ph | S | methyl | methyl | H |
| 2-21-1148 | N—Ph | S | b1 | b1 | H |
| 2-21-1149 | N—Ph | S | b2 | b2 | H |
| 2-21-1150 | N—Ph | S | b3 | b3 | H |
| 2-21-1151 | N—Ph | S | H | H | H |
| 2-21-1152 | N—Ph | S | t-bu | t-bu | H |
| 2-21-1153 | N—Ph | S | methyl | methyl | H |
| 2-21-1154 | N—Ph | S | b1 | b1 | H |
| 2-21-1155 | N—Ph | S | b2 | b2 | H |
| 2-21-1156 | N—Ph | S | b3 | b3 | H |
| 2-21-1157 | N—Ph | S | H | H | H |
| 2-21-1158 | N—Ph | S | t-bu | t-bu | H |
| 2-21-1159 | N—Ph | S | methyl | methyl | H |
| 2-21-1160 | N—Ph | S | b1 | b1 | H |
| 2-21-1161 | N—Ph | S | b2 | b2 | H |
| 2-21-1162 | N—Ph | S | b3 | b3 | H |
| 2-21-1163 | N—Ph | S | H | H | H |
| 2-21-1164 | N—Ph | S | t-bu | t-bu | H |
| 2-21-1165 | N—Ph | S | methyl | methyl | H |
| 2-21-1166 | N—Ph | S | b1 | b1 | H |
| 2-21-1167 | N—Ph | S | b2 | b2 | H |
| 2-21-1168 | N—Ph | S | b3 | b3 | H |
| 2-21-1169 | N—Ph | S | H | H | H |
| 2-21-1170 | N—Ph | S | t-bu | t-bu | H |
| 2-21-1171 | N—Ph | S | methyl | methyl | H |
| 2-21-1172 | N—Ph | S | b1 | b1 | H |
| 2-21-1173 | N—Ph | S | b2 | b2 | H |
| 2-21-1174 | N—Ph | S | b3 | b3 | H |
| 2-21-1175 | N—Ph | S | H | H | H |
| 2-21-1176 | N—Ph | S | t-bu | t-bu | H |
| 2-21-1177 | N—Ph | S | methyl | methyl | H |
| 2-21-1178 | N—Ph | S | b1 | b1 | H |
| 2-21-1179 | N—Ph | S | b2 | b2 | H |
| 2-21-1180 | N—Ph | S | b3 | b3 | H |
| 2-21-1181 | N—Ph | S | H | H | H |
| 2-21-1182 | N—Ph | S | t-bu | t-bu | H |
| 2-21-1183 | N—Ph | S | methyl | methyl | H |
| 2-21-1184 | N—Ph | S | b1 | b1 | H |
| 2-21-1185 | N—Ph | S | b2 | b2 | H |
| 2-21-1186 | N—Ph | S | b3 | b3 | H |
| 2-21-1187 | N—Ph | S | H | H | H |
| 2-21-1188 | N—Ph | S | t-bu | t-bu | H |
| 2-21-1189 | N—Ph | S | methyl | methyl | H |
| 2-21-1190 | N—Ph | S | b1 | b1 | H |
| 2-21-1191 | N—Ph | S | b2 | b2 | H |
| 2-21-1192 | N—Ph | S | b3 | b3 | H |
| 2-21-1193 | N—Ph | S | H | H | H |
| 2-21-1194 | N—Ph | S | t-bu | t-bu | H |
| 2-21-1195 | N—Ph | S | methyl | methyl | H |
| 2-21-1196 | N—Ph | S | b1 | b1 | H |
| 2-21-1197 | N—Ph | S | b2 | b2 | H |
| 2-21-1198 | N—Ph | S | b3 | b3 | H |
| 2-21-1199 | N—Ph | S | H | H | H |
| 2-21-1200 | N—Ph | S | t-bu | t-bu | H |
| 2-21-1201 | N—Ph | S | methyl | methyl | H |
| 2-21-1202 | N—Ph | S | b1 | b1 | H |
| 2-21-1203 | N—Ph | S | b2 | b2 | H |
| 2-21-1204 | N—Ph | S | b3 | b3 | H |
| 2-21-1205 | N—Ph | S | H | H | H |
| 2-21-1206 | N—Ph | S | t-bu | t-bu | H |
| 2-21-1207 | N—Ph | S | methyl | methyl | H |
| 2-21-1208 | N—Ph | S | b1 | b1 | H |
| 2-21-1209 | N—Ph | S | b2 | b2 | H |
| 2-21-1210 | N—Ph | S | b3 | b3 | H |
| 2-21-1211 | N—Ph | S | H | H | H |
| 2-21-1212 | N—Ph | S | t-bu | t-bu | H |
| 2-21-1213 | N—Ph | S | methyl | methyl | H |
| 2-21-1214 | N—Ph | S | b1 | b1 | H |
| 2-21-1215 | N—Ph | S | b2 | b2 | H |
| 2-21-1216 | N—Ph | S | b3 | b3 | H |
| 2-21-1217 | N—Ph | S | H | H | a1 |
| 2-21-1218 | N—Ph | S | t-bu | t-bu | a1 |
| 2-21-1219 | N—Ph | S | methyl | methyl | a1 |
| 2-21-1220 | N—Ph | S | b1 | b1 | a1 |
| 2-21-1221 | N—Ph | S | b2 | b2 | a1 |
| 2-21-1222 | N—Ph | S | b3 | b3 | a1 |
| 2-21-1223 | N—Ph | S | H | H | a2 |
| 2-21-1224 | N—Ph | S | t-bu | t-bu | a2 |
| 2-21-1225 | N—Ph | S | methyl | methyl | a2 |
| 2-21-1226 | N—Ph | S | b1 | b1 | a2 |
| 2-21-1227 | N—Ph | S | b2 | b2 | a2 |
| 2-21-1228 | N—Ph | S | b3 | b3 | a2 |
| 2-21-1229 | N—Ph | S | H | H | a3 |
| 2-21-1230 | N—Ph | S | t-bu | t-bu | a3 |
| 2-21-1231 | N—Ph | S | methyl | methyl | a3 |
| 2-21-1232 | N—Ph | S | b1 | b1 | a3 |
| 2-21-1233 | N—Ph | S | b2 | b2 | a3 |
| 2-21-1234 | N—Ph | S | b3 | b3 | a3 |
| 2-21-1235 | N—Ph | S | H | H | b1 |
| 2-21-1236 | N—Ph | S | t-bu | t-bu | b1 |
| 2-21-1237 | N—Ph | S | methyl | methyl | b1 |
| 2-21-1238 | N—Ph | S | b1 | b1 | b1 |
| 2-21-1239 | N—Ph | S | b2 | b2 | b1 |
| 2-21-1240 | N—Ph | S | b3 | b3 | b1 |
| 2-21-1241 | N—Ph | S | H | H | b2 |
| 2-21-1242 | N—Ph | S | t-bu | t-bu | b2 |
| 2-21-1243 | N—Ph | S | methyl | methyl | b2 |
| 2-21-1244 | N—Ph | S | b1 | b1 | b2 |
| 2-21-1245 | N—Ph | S | b2 | b2 | b2 |
| 2-21-1246 | N—Ph | S | b3 | b3 | b2 |
| 2-21-1247 | N—Ph | S | H | H | b3 |
| 2-21-1248 | N—Ph | S | t-bu | t-bu | b3 |
| 2-21-1249 | N—Ph | S | methyl | methyl | b3 |
| 2-21-1250 | N—Ph | S | b1 | b1 | b3 |
| 2-21-1251 | N—Ph | S | b2 | b2 | b3 |
| 2-21-1252 | N—Ph | S | b3 | b3 | b3 |
| 2-21-1253 | N—Ph | S | H | H | b5 |
| 2-21-1254 | N—Ph | S | t-bu | t-bu | b5 |
| 2-21-1255 | N—Ph | S | methyl | methyl | b5 |
| 2-21-1256 | N—Ph | S | b1 | b1 | b5 |
| 2-21-1257 | N—Ph | S | b2 | b2 | b5 |
| 2-21-1258 | N—Ph | S | b3 | b3 | b5 |
| 2-21-1259 | N—Ph | S | H | H | b6 |
| 2-21-1260 | N—Ph | S | t-bu | t-bu | b6 |
| 2-21-1261 | N—Ph | S | methyl | methyl | b6 |
| 2-21-1262 | N—Ph | S | b1 | b1 | b6 |
| 2-21-1263 | N—Ph | S | b2 | b2 | b6 |
| 2-21-1264 | N—Ph | S | b3 | b3 | b6 |
| 2-21-1265 | N—Ph | S | H | H | b7 |
| 2-21-1266 | N—Ph | S | t-bu | t-bu | b7 |
| 2-21-1267 | N—Ph | S | methyl | methyl | b7 |
| 2-21-1268 | N—Ph | S | b1 | b1 | b7 |
| 2-21-1269 | N—Ph | S | b2 | b2 | b7 |
| 2-21-1270 | N—Ph | S | b3 | b3 | b7 |

Group 2-22
The compounds are represented by the formula 2-22 below, wherein CY₂ is a benzene group, X₁ is B, and Y1, Y2, Y3, R1b, R3a, (R2)a2 are each shown in the table 22 below, 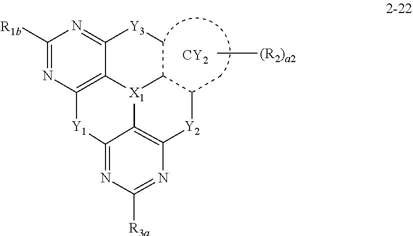
TABLE 22

| Compound | | | | | |
|---|---|---|---|---|---|
| No. | Y1 | Y2/Y3 | R1b | R3a | (R2)a2 |
| 2-22-001 | N-Ph | N-Ph | H | H | H |
| 2-22-002 | N-Ph | N-Ph | t-bu | t-bu | H |
| 2-22-003 | N-Ph | N-Ph | methyl | methyl | H |
| 2-22-004 | N-Ph | N-Ph | t-bu | t-bu | t-bu |
| 2-22-005 | N-Ph | N-Ph | methyl | methyl | methyl |
| 2-22-006 | N-Ph | N-Ph | b1 | b1 | H |
| 2-22-007 | N-Ph | N-Ph | b2 | b2 | H |
| 2-22-008 | N-Ph | N-Ph | b3 | b3 | H |
| 2-22-009 | N-Ph | N-Ph | H | H | a1 |
| 2-22-010 | N-Ph | N-Ph | t-bu | t-bu | a1 |
| 2-22-011 | N-Ph | N-Ph | methyl | methyl | a1 |
| 2-22-012 | N-Ph | N-Ph | b1 | b1 | a1 |
| 2-22-013 | N-Ph | N-Ph | b2 | b2 | a1 |
| 2-22-014 | N-Ph | N-Ph | b3 | b3 | a1 |
| 2-22-015 | N-Ph | N-Ph | H | H | a2 |
| 2-22-016 | N-Ph | N-Ph | t-bu | t-bu | a2 |
| 2-22-017 | N-Ph | N-Ph | methyl | methyl | a2 |
| 2-22-018 | N-Ph | N-Ph | b1 | b1 | a2 |
| 2-22-019 | N-Ph | N-Ph | b2 | b2 | a2 |
| 2-22-020 | N-Ph | N-Ph | b3 | b3 | a2 |
| 2-22-021 | N-Ph | N-Ph | H | H | a3 |
| 2-22-022 | N-Ph | N-Ph | t-bu | t-bu | a3 |
| 2-22-023 | N-Ph | N-Ph | methyl | methyl | a3 |
| 2-22-024 | N-Ph | N-Ph | b1 | b1 | a3 |
| 2-22-025 | N-Ph | N-Ph | b2 | b2 | a3 |
| 2-22-026 | N-Ph | N-Ph | b3 | b3 | a3 |
| 2-22-027 | N-Ph | N-Ph | H | H | a4 |
| 2-22-028 | N-Ph | N-Ph | t-bu | t-bu | a4 |
| 2-22-029 | N-Ph | N-Ph | methyl | methyl | a4 |
| 2-22-030 | N-Ph | N-Ph | b1 | b1 | a4 |
| 2-22-031 | N-Ph | N-Ph | b2 | b2 | a4 |
| 2-22-032 | N-Ph | N-Ph | b3 | b3 | a4 |
| 2-22-033 | N-Ph | N-Ph | H | H | a5 |
| 2-22-034 | N-Ph | N-Ph | t-bu | t-bu | a5 |
| 2-22-035 | N-Ph | N-Ph | methyl | methyl | a5 |
| 2-22-036 | N-Ph | N-Ph | b1 | b1 | a5 |
| 2-22-037 | N-Ph | N-Ph | b2 | b2 | a5 |
| 2-22-038 | N-Ph | N-Ph | b3 | b3 | a5 |
| 2-22-039 | N-Ph | N-Ph | H | H | a6 |
| 2-22-040 | N-Ph | N-Ph | t-bu | t-bu | a6 |
| 2-22-041 | N-Ph | N-Ph | methyl | methyl | a6 |
| 2-22-042 | N-Ph | N-Ph | b1 | b1 | a6 |
| 2-22-043 | N-Ph | N-Ph | b2 | b2 | a6 |
| 2-22-044 | N-Ph | N-Ph | b3 | b3 | a6 |
| 2-22-045 | N-Ph | N-Ph | H | H | b1 |
| 2-22-046 | N-Ph | N-Ph | t-bu | t-bu | b1 |
| 2-22-047 | N-Ph | N-Ph | methyl | methyl | b1 |
| 2-22-048 | N-Ph | N-Ph | b1 | b1 | b1 |
| 2-22-049 | N-Ph | N-Ph | b2 | b2 | b1 |
| 2-22-050 | N-Ph | N-Ph | b3 | b3 | b1 |
| 2-22-051 | N-Ph | N-Ph | H | H | b2 |
| 2-22-052 | N-Ph | N-Ph | t-bu | t-bu | b2 |
| 2-22-053 | N-Ph | N-Ph | methyl | methyl | b2 |
| 2-22-054 | N-Ph | N-Ph | b1 | b1 | b2 |
| 2-22-055 | N-Ph | N-Ph | b2 | b2 | b2 |
| 2-22-056 | N-Ph | N-Ph | b3 | b3 | b2 |
| 2-22-057 | N-Ph | N-Ph | H | H | b3 |
| 2-22-058 | N-Ph | N-Ph | t-bu | t-bu | b3 |
| 2-22-059 | N-Ph | N-Ph | methyl | methyl | b3 |
| 2-22-060 | N-Ph | N-Ph | b1 | b1 | b3 |
| 2-22-061 | N-Ph | N-Ph | b2 | b2 | b3 |
| 2-22-062 | N-Ph | N-Ph | b3 | b3 | b3 |
| 2-22-063 | N-Ph | N-Ph | H | H | b4 |
| 2-22-064 | N-Ph | N-Ph | t-bu | t-bu | b4 |
| 2-22-065 | N-Ph | N-Ph | methyl | methyl | b4 |
| 2-22-066 | N-Ph | N-Ph | b1 | b1 | b4 |
| 2-22-067 | N-Ph | N-Ph | b2 | b2 | b4 |
| 2-22-068 | N-Ph | N-Ph | b3 | b3 | b4 |
| 2-22-069 | N-Ph | N-Ph | H | H | b5 |
| 2-22-070 | N-Ph | N-Ph | t-bu | t-bu | b5 |
| 2-22-071 | N-Ph | N-Ph | methyl | methyl | b5 |
| 2-22-072 | N-Ph | N-Ph | b1 | b1 | b5 |
| 2-22-073 | N-Ph | N-Ph | b2 | b2 | b5 |
| 2-22-074 | N-Ph | N-Ph | b3 | b3 | b5 |
| 2-22-075 | N-Ph | N-Ph | H | H | b6 |
| 2-22-076 | N-Ph | N-Ph | t-bu | t-bu | b6 |
| 2-22-077 | N-Ph | N-Ph | methyl | methyl | b6 |
| 2-22-078 | N-Ph | N-Ph | b1 | b1 | b6 |
| 2-22-079 | N-Ph | N-Ph | b2 | b2 | b6 |
| 2-22-080 | N-Ph | N-Ph | b3 | b3 | b6 |
| 2-22-081 | N-Ph | N-Ph | H | H | b7 |
| 2-22-082 | N-Ph | N-Ph | t-bu | t-bu | b7 |
| 2-22-083 | N-Ph | N-Ph | methyl | methyl | b7 |
| 2-22-084 | N-Ph | N-Ph | b1 | b1 | b7 |
| 2-22-085 | N-Ph | N-Ph | b2 | b2 | b7 |
| 2-22-086 | N-Ph | N-Ph | b3 | b3 | b7 |
| 2-22-087 | N-Ph | N-Ph | H | H | b8 |
| 2-22-088 | N-Ph | N-Ph | t-bu | t-bu | b8 |
| 2-22-089 | N-Ph | N-Ph | methyl | methyl | b8 |
| 2-22-090 | N-Ph | N-Ph | b1 | b1 | b8 |
| 2-22-091 | N-Ph | N-Ph | b2 | b2 | b8 |
| 2-22-092 | N-Ph | N-Ph | b3 | b3 | b8 |
| 2-22-093 | N-Ph | N-Ph | H | H | b9 |
| 2-22-094 | N-Ph | N-Ph | t-bu | t-bu | b9 |
| 2-22-095 | N-Ph | N-Ph | methyl | methyl | b9 |
| 2-22-096 | N-Ph | N-Ph | b1 | b1 | b9 |
| 2-22-097 | N-Ph | N-Ph | b2 | b2 | b9 |
| 2-22-098 | N-Ph | N-Ph | b3 | b3 | b9 |
| 2-22-099 | N-Ph | N-Ph | H | H | b10 |
| 2-22-100 | N-Ph | N-Ph | t-bu | t-bu | b10 |
| 2-22-101 | N-Ph | N-Ph | methyl | methyl | b10 |
| 2-22-102 | N-Ph | N-Ph | b1 | b1 | b10 |
| 2-22-103 | N-Ph | N-Ph | b2 | b2 | b10 |
| 2-22-104 | N-Ph | N-Ph | b3 | b3 | b10 |
| 2-22-105 | N-Ph | N-Ph | H | H | H |
| 2-22-106 | N-Ph | N-Ph | t-bu | t-bu | H |
| 2-22-107 | N-Ph | N-Ph | methyl | methyl | H |
| 2-22-108 | N-Ph | N-Ph | b1 | b1 | H |
| 2-22-109 | N-Ph | N-Ph | b2 | b2 | H |
| 2-22-110 | N-Ph | N-Ph | b3 | b3 | H |
| 2-22-111 | N-Ph | N-Ph | H | H | H |
| 2-22-112 | N-Ph | N-Ph | t-bu | t-bu | H |
| 2-22-113 | N-Ph | N-Ph | methyl | methyl | H |
| 2-22-114 | N-Ph | N-Ph | b1 | b1 | H |
| 2-22-115 | N-Ph | N-Ph | b2 | b2 | H |
| 2-22-116 | N-Ph | N-Ph | b3 | b3 | H |
| 2-22-117 | N-Ph | N-Ph | H | H | H |
| 2-22-118 | N-Ph | N-Ph | t-bu | t-bu | H |
| 2-22-119 | N-Ph | N-Ph | methyl | methyl | H |
| 2-22-120 | N-Ph | N-Ph | b1 | b1 | H |
| 2-22-121 | N-Ph | N-Ph | b2 | b2 | H |
| 2-22-122 | N-Ph | N-Ph | b3 | b3 | H |
| 2-22-123 | N-Ph | N-Ph | H | H | H |
| 2-22-124 | N-Ph | N-Ph | t-bu | t-bu | H |
| 2-22-125 | N-Ph | N-Ph | methyl | methyl | H |
| 2-22-126 | N-Ph | N-Ph | b1 | b1 | H |
| 2-22-127 | N-Ph | N-Ph | b2 | b2 | H |
| 2-22-128 | N-Ph | N-Ph | b3 | b3 | H |
| 2-22-129 | N-Ph | N-Ph | H | H | H |
| 2-22-130 | N-Ph | N-Ph | t-bu | t-bu | H |
| 2-22-131 | N-Ph | N-Ph | methyl | methyl | H |
| 2-22-132 | N-Ph | N-Ph | b1 | b1 | H |
| 2-22-133 | N-Ph | N-Ph | b2 | b2 | H |
| 2-22-134 | N-Ph | N-Ph | b3 | b3 | H |
| 2-22-135 | N-Ph | N-Ph | H | H | H |
| 2-22-136 | N-Ph | N-Ph | t-bu | t-bu | H |
| 2-22-137 | N-Ph | N-Ph | methyl | methyl | H |
| 2-22-138 | N-Ph | N-Ph | b1 | b1 | H |
| 2-22-139 | N-Ph | N-Ph | b2 | b2 | H |
| 2-22-140 | N-Ph | N-Ph | b3 | b3 | H |
| 2-22-141 | N-Ph | N-Ph | H | H | H |
| 2-22-142 | N-Ph | N-Ph | t-bu | t-bu | H |
| 2-22-143 | N-Ph | N-Ph | methyl | methyl | H |
| 2-22-144 | N-Ph | N-Ph | b1 | b1 | H |
| 2-22-145 | N-Ph | N-Ph | b2 | b2 | H |
| 2-22-146 | N-Ph | N-Ph | b3 | b3 | H |
| 2-22-147 | N-Ph | N-Ph | H | H | H |
| 2-22-148 | N-Ph | N-Ph | t-bu | t-bu | H |
| 2-22-149 | N-Ph | N-Ph | methyl | methyl | H |
| 2-22-150 | N-Ph | N-Ph | b1 | b1 | H |
| 2-22-151 | N-Ph | N-Ph | b2 | b2 | H |
| 2-22-152 | N-Ph | N-Ph | b3 | b3 | H |
| 2-22-153 | N-Ph | N-Ph | H | H | H |
| 2-22-154 | N-Ph | N-Ph | t-bu | t-bu | H |
| 2-22-155 | N-Ph | N-Ph | methyl | methyl | H |
| 2-22-156 | N-Ph | N-Ph | b1 | b1 | H |
| 2-22-157 | N-Ph | N-Ph | b2 | b2 | H |
| 2-22-158 | N-Ph | N-Ph | b3 | b3 | H |
| 2-22-159 | N-Ph | N-Ph | H | H | H |
| 2-22-160 | N-Ph | N-Ph | t-bu | t-bu | H |
| 2-22-161 | N-Ph | N-Ph | methyl | methyl | H |
| 2-22-162 | N-Ph | N-Ph | b1 | b1 | H |
| 2-22-163 | N-Ph | N-Ph | b2 | b2 | H |
| 2-22-164 | N-Ph | N-Ph | b3 | b3 | H |
| 2-22-165 | N-Ph | N-Ph | H | H | H |
| 2-22-166 | N-Ph | N-Ph | t-bu | t-bu | H |
| 2-22-167 | N-Ph | N-Ph | methyl | methyl | H |
| 2-22-168 | N-Ph | N-Ph | b1 | b1 | H |
| 2-22-169 | N-Ph | N-Ph | b2 | b2 | H |
| 2-22-170 | N-Ph | N-Ph | b3 | b3 | H |
| 2-22-171 | N-Ph | N-Ph | H | H | H |
| 2-22-172 | N-Ph | N-Ph | t-bu | t-bu | H |
| 2-22-173 | N-Ph | N-Ph | methyl | methyl | H |
| 2-22-174 | N-Ph | N-Ph | b1 | b1 | H |
| 2-22-175 | N-Ph | N-Ph | b2 | b2 | H |
| 2-22-176 | N-Ph | N-Ph | b3 | b3 | H |
| 2-22-177 | N-Ph | N-Ph | H | H | H |
| 2-22-178 | N-Ph | N-Ph | t-bu | t-bu | H |
| 2-22-179 | N-Ph | N-Ph | methyl | methyl | H |
| 2-22-180 | N-Ph | N-Ph | b1 | b1 | H |
| 2-22-181 | N-Ph | N-Ph | b2 | b2 | H |
| 2-22-182 | N-Ph | N-Ph | b3 | b3 | H |
| 2-22-183 | N-Ph | N-Ph | H | H | H |
| 2-22-184 | N-Ph | N-Ph | t-bu | t-bu | H |
| 2-22-185 | N-Ph | N-Ph | methyl | methyl | H |
| 2-22-186 | N-Ph | N-Ph | b1 | b1 | H |
| 2-22-187 | N-Ph | N-Ph | b2 | b2 | H |
| 2-22-188 | N-Ph | N-Ph | b3 | b3 | H |
| 2-22-189 | N-Ph | N-Ph | H | H | H |
| 2-22-190 | N-Ph | N-Ph | t-bu | t-bu | H |
| 2-22-191 | N-Ph | N-Ph | methyl | methyl | H |
| 2-22-192 | N-Ph | N-Ph | b1 | b1 | H |
| 2-22-193 | N-Ph | N-Ph | b2 | b2 | H |
| 2-22-194 | N-Ph | N-Ph | b3 | b3 | H |
| 2-22-195 | N-Ph | N-Ph | H | H | H |
| 2-22-196 | N-Ph | N-Ph | t-bu | t-bu | H |
| 2-22-197 | N-Ph | N-Ph | methyl | methyl | H |
| 2-22-198 | N-Ph | N-Ph | b1 | b1 | H |
| 2-22-199 | N-Ph | N-Ph | b2 | b2 | H |
| 2-22-200 | N-Ph | N-Ph | b3 | b3 | H |
| 2-22-201 | N-Ph | N-Ph | H | H | a1 |
| 2-22-202 | N-Ph | N-Ph | t-bu | t-bu | a1 |
| 2-22-203 | N-Ph | N-Ph | methyl | methyl | a1 |
| 2-22-204 | N-Ph | N-Ph | b1 | b1 | a1 |
| 2-22-205 | N-Ph | N-Ph | b2 | b2 | a1 |
| 2-22-206 | N-Ph | N-Ph | b3 | b3 | a1 |
| 2-22-207 | N-Ph | N-Ph | H | H | a2 |
| 2-22-208 | N-Ph | N-Ph | t-bu | t-bu | a2 |
| 2-22-209 | N-Ph | N-Ph | methyl | methyl | a2 |
| 2-22-210 | N-Ph | N-Ph | b1 | b1 | a2 |
| 2-22-211 | N-Ph | N-Ph | b2 | b2 | a2 |
| 2-22-212 | N-Ph | N-Ph | b3 | b3 | a2 |
| 2-22-213 | N-Ph | N-Ph | H | H | a3 |
| 2-22-214 | N-Ph | N-Ph | t-bu | t-bu | a3 |
| 2-22-215 | N-Ph | N-Ph | methyl | methyl | a3 |
| 2-22-216 | N-Ph | N-Ph | b1 | b1 | a3 |
| 2-22-217 | N-Ph | N-Ph | b2 | b2 | a3 |
| 2-22-218 | N-Ph | N-Ph | b3 | b3 | a3 |
| 2-22-219 | N-Ph | N-Ph | H | H | b1 |
| 2-22-220 | N-Ph | N-Ph | t-bu | t-bu | b1 |
| 2-22-221 | N-Ph | N-Ph | methyl | methyl | b1 |
| 2-22-222 | N-Ph | N-Ph | b1 | b1 | b1 |
| 2-22-223 | N-Ph | N-Ph | b2 | b2 | b1 |
| 2-22-224 | N-Ph | N-Ph | b3 | b3 | b1 |
| 2-22-225 | N-Ph | N-Ph | H | H | b2 |
| 2-22-226 | N-Ph | N-Ph | t-bu | t-bu | b2 |
| 2-22-227 | N-Ph | N-Ph | methyl | methyl | b2 |
| 2-22-228 | N-Ph | N-Ph | b1 | b1 | b2 |
| 2-22-229 | N-Ph | N-Ph | b2 | b2 | b2 |
| 2-22-230 | N-Ph | N-Ph | b3 | b3 | b2 |
| 2-22-231 | N-Ph | N-Ph | H | H | b3 |
| 2-22-232 | N-Ph | N-Ph | t-bu | t-bu | b3 |
| 2-22-233 | N-Ph | N-Ph | methyl | methyl | b3 |
| 2-22-234 | N-Ph | N-Ph | b1 | b1 | b3 |
| 2-22-235 | N-Ph | N-Ph | b2 | b2 | b3 |
| 2-22-236 | N-Ph | N-Ph | b3 | b3 | b3 |
| 2-22-237 | N-Ph | N-Ph | H | H | b5 |
| 2-22-238 | N-Ph | N-Ph | t-bu | t-bu | b5 |
| 2-22-239 | N-Ph | N-Ph | methyl | methyl | b5 |
| 2-22-240 | N-Ph | N-Ph | b1 | b1 | b5 |
| 2-22-241 | N-Ph | N-Ph | b2 | b2 | b5 |
| 2-22-242 | N-Ph | N-Ph | b3 | b3 | b5 |
| 2-22-243 | N-Ph | N-Ph | H | H | b6 |
| 2-22-244 | N-Ph | N-Ph | t-bu | t-bu | b6 |
| 2-22-245 | N-Ph | N-Ph | methyl | methyl | b6 |
| 2-22-246 | N-Ph | N-Ph | b1 | b1 | b6 |
| 2-22-247 | N-Ph | N-Ph | b2 | b2 | b6 |
| 2-22-248 | N-Ph | N-Ph | b3 | b3 | b6 |
| 2-22-249 | N-Ph | N-Ph | H | H | b7 |
| 2-22-250 | N-Ph | N-Ph | t-bu | t-bu | b7 |
| 2-22-251 | N-Ph | N-Ph | methyl | methyl | b7 |
| 2-22-252 | N-Ph | N-Ph | b1 | b1 | b7 |
| 2-22-253 | N-Ph | N-Ph | b2 | b2 | b7 |
| 2-22-254 | N-Ph | N-Ph | b3 | b3 | b7 |
| 2-22-255 | N-tolyl | N-tolyl | H | H | H |
| 2-22-256 | N-tolyl | N-tolyl | t-bu | t-bu | H |
| 2-22-257 | N-tolyl | N-tolyl | methyl | methyl | H |
| 2-22-258 | N-tolyl | N-tolyl | t-bu | t-bu | t-bu |
| 2-22-259 | N-tolyl | N-tolyl | methyl | methyl | methyl |
| 2-22-260 | N-tolyl | N-tolyl | b1 | b1 | H |
| 2-22-261 | N-tolyl | N-tolyl | b2 | b2 | H |
| 2-22-262 | N-tolyl | N-tolyl | b3 | b3 | H |
| 2-22-263 | N-tolyl | N-tolyl | H | H | a1 |
| 2-22-264 | N-tolyl | N-tolyl | t-bu | t-bu | a1 |
| 2-22-265 | N-tolyl | N-tolyl | methyl | methyl | a1 |
| 2-22-266 | N-tolyl | N-tolyl | b1 | b1 | a1 |
| 2-22-267 | N-tolyl | N-tolyl | b2 | b2 | a1 |
| 2-22-268 | N-tolyl | N-tolyl | b3 | b3 | a1 |
| 2-22-269 | N-tolyl | N-tolyl | H | H | a2 |
| 2-22-270 | N-tolyl | N-tolyl | t-bu | t-bu | a2 |
| 2-22-271 | N-tolyl | N-tolyl | methyl | methyl | a2 |
| 2-22-272 | N-tolyl | N-tolyl | b1 | b1 | a2 |
| 2-22-273 | N-tolyl | N-tolyl | b2 | b2 | a2 |
| 2-22-274 | N-tolyl | N-tolyl | b3 | b3 | a2 |
| 2-22-275 | N-tolyl | N-tolyl | H | H | a3 |
| 2-22-276 | N-tolyl | N-tolyl | t-bu | t-bu | a3 |
| 2-22-277 | N-tolyl | N-tolyl | methyl | methyl | a3 |
| 2-22-278 | N-tolyl | N-tolyl | b1 | b1 | a3 |
| 2-22-279 | N-tolyl | N-tolyl | b2 | b2 | a3 |
| 2-22-280 | N-tolyl | N-tolyl | b3 | b3 | a3 |
| 2-22-281 | N-tolyl | N-tolyl | H | H | a4 |
| 2-22-282 | N-tolyl | N-tolyl | t-bu | t-bu | a4 |
| 2-22-283 | N-tolyl | N-tolyl | methyl | methyl | a4 |
| 2-22-284 | N-tolyl | N-tolyl | b1 | b1 | a4 |
| 2-22-285 | N-tolyl | N-tolyl | b2 | b2 | a4 |
| 2-22-286 | N-tolyl | N-tolyl | b3 | b3 | a4 |
| 2-22-287 | N-tolyl | N-tolyl | H | H | a5 |
| 2-22-288 | N-tolyl | N-tolyl | t-bu | t-bu | a5 |
| 2-22-289 | N-tolyl | N-tolyl | methyl | methyl | a5 |
| 2-22-290 | N-tolyl | N-tolyl | b1 | b1 | a5 |
| 2-22-291 | N-tolyl | N-tolyl | b2 | b2 | a5 |
| 2-22-292 | N-tolyl | N-tolyl | b3 | b3 | a5 |
| 2-22-293 | N-tolyl | N-tolyl | H | H | a6 |
| 2-22-294 | N-tolyl | N-tolyl | t-bu | t-bu | a6 |
| 2-22-295 | N-tolyl | N-tolyl | methyl | methyl | a6 |
| 2-22-296 | N-tolyl | N-tolyl | b1 | b1 | a6 |
| 2-22-297 | N-tolyl | N-tolyl | b2 | b2 | a6 |
| 2-22-298 | N-tolyl | N-tolyl | b3 | b3 | a6 |
| 2-22-299 | N-tolyl | N-tolyl | H | H | b1 |
| 2-22-300 | N-tolyl | N-tolyl | t-bu | t-bu | b1 |
| 2-22-301 | N-tolyl | N-tolyl | methyl | methyl | b1 |
| 2-22-302 | N-tolyl | N-tolyl | b1 | b1 | b1 |
| 2-22-303 | N-tolyl | N-tolyl | b2 | b2 | b1 |
| 2-22-304 | N-tolyl | N-tolyl | b3 | b3 | b1 |
| 2-22-305 | N-tolyl | N-tolyl | H | H | b2 |
| 2-22-306 | N-tolyl | N-tolyl | t-bu | t-bu | b2 |
| 2-22-307 | N-tolyl | N-tolyl | methyl | methyl | b2 |
| 2-22-308 | N-tolyl | N-tolyl | b1 | b1 | b2 |
| 2-22-309 | N-tolyl | N-tolyl | b2 | b2 | b2 |
| 2-22-310 | N-tolyl | N-tolyl | b3 | b3 | b2 |
| 2-22-311 | N-tolyl | N-tolyl | H | H | b3 |
| 2-22-312 | N-tolyl | N-tolyl | t-bu | t-bu | b3 |
| 2-22-313 | N-tolyl | N-tolyl | methyl | methyl | b3 |
| 2-22-314 | N-tolyl | N-tolyl | b1 | b1 | b3 |
| 2-22-315 | N-tolyl | N-tolyl | b2 | b2 | b3 |
| 2-22-316 | N-tolyl | N-tolyl | b3 | b3 | b3 |
| 2-22-317 | N-tolyl | N-tolyl | H | H | b4 |
| 2-22-318 | N-tolyl | N-tolyl | t-bu | t-bu | b4 |
| 2-22-319 | N-tolyl | N-tolyl | methyl | methyl | b4 |
| 2-22-320 | N-tolyl | N-tolyl | b1 | b1 | b4 |
| 2-22-321 | N-tolyl | N-tolyl | b2 | b2 | b4 |
| 2-22-322 | N-tolyl | N-tolyl | b3 | b3 | b4 |
| 2-22-323 | N-tolyl | N-tolyl | H | H | b5 |
| 2-22-324 | N-tolyl | N-tolyl | t-bu | t-bu | b5 |
| 2-22-325 | N-tolyl | N-tolyl | methyl | methyl | b5 |
| 2-22-326 | N-tolyl | N-tolyl | b1 | b1 | b5 |
| 2-22-327 | N-tolyl | N-tolyl | b2 | b2 | b5 |
| 2-22-328 | N-tolyl | N-tolyl | b3 | b3 | b5 |
| 2-22-329 | N-tolyl | N-tolyl | H | H | b6 |
| 2-22-330 | N-tolyl | N-tolyl | t-bu | t-bu | b6 |
| 2-22-331 | N-tolyl | N-tolyl | methyl | methyl | b6 |
| 2-22-332 | N-tolyl | N-tolyl | b1 | b1 | b6 |
| 2-22-333 | N-tolyl | N-tolyl | b2 | b2 | b6 |
| 2-22-334 | N-tolyl | N-tolyl | b3 | b3 | b6 |
| 2-22-335 | N-tolyl | N-tolyl | H | H | b7 |
| 2-22-336 | N-tolyl | N-tolyl | t-bu | t-bu | b7 |
| 2-22-337 | N-tolyl | N-tolyl | methyl | methyl | b7 |
| 2-22-338 | N-tolyl | N-tolyl | b1 | b1 | b7 |
| 2-22-339 | N-tolyl | N-tolyl | b2 | b2 | b7 |
| 2-22-340 | N-tolyl | N-tolyl | b3 | b3 | b7 |
| 2-22-341 | N-tolyl | N-tolyl | H | H | b8 |
| 2-22-342 | N-tolyl | N-tolyl | t-bu | t-bu | b8 |
| 2-22-343 | N-tolyl | N-tolyl | methyl | methyl | b8 |
| 2-22-344 | N-tolyl | N-tolyl | b1 | b1 | b8 |
| 2-22-345 | N-tolyl | N-tolyl | b2 | b2 | b8 |
| 2-22-346 | N-tolyl | N-tolyl | b3 | b3 | b8 |
| 2-22-347 | N-tolyl | N-tolyl | H | H | b9 |
| 2-22-348 | N-tolyl | N-tolyl | t-bu | t-bu | b9 |
| 2-22-349 | N-tolyl | N-tolyl | methyl | methyl | b9 |
| 2-22-350 | N-tolyl | N-tolyl | b1 | b1 | b9 |
| 2-22-351 | N-tolyl | N-tolyl | b2 | b2 | b9 |
| 2-22-352 | N-tolyl | N-tolyl | b3 | b3 | b9 |
| 2-22-353 | N-tolyl | N-tolyl | H | H | b10 |
| 2-22-354 | N-tolyl | N-tolyl | t-bu | t-bu | b10 |
| 2-22-355 | N-tolyl | N-tolyl | methyl | methyl | b10 |
| 2-22-356 | N-tolyl | N-tolyl | b1 | b1 | b10 |
| 2-22-357 | N-tolyl | N-tolyl | b2 | b2 | b10 |
| 2-22-358 | N-tolyl | N-tolyl | b3 | b3 | b10 |
| 2-22-359 | N-tolyl | N-tolyl | H | H | H |
| 2-22-360 | N-tolyl | N-tolyl | t-bu | t-bu | H |
| 2-22-361 | N-tolyl | N-tolyl | methyl | methyl | H |
| 2-22-362 | N-tolyl | N-tolyl | b1 | b1 | H |
| 2-22-363 | N-tolyl | N-tolyl | b2 | b2 | H |
| 2-22-364 | N-tolyl | N-tolyl | b3 | b3 | H |
| 2-22-365 | N-tolyl | N-tolyl | H | H | H |
| 2-22-366 | N-tolyl | N-tolyl | t-bu | t-bu | H |
| 2-22-367 | N-tolyl | N-tolyl | methyl | methyl | H |
| 2-22-368 | N-tolyl | N-tolyl | b1 | b1 | H |
| 2-22-369 | N-tolyl | N-tolyl | b2 | b2 | H |
| 2-22-370 | N-tolyl | N-tolyl | b3 | b3 | H |
| 2-22-371 | N-tolyl | N-tolyl | H | H | H |
| 2-22-372 | N-tolyl | N-tolyl | t-bu | t-bu | H |
| 2-22-373 | N-tolyl | N-tolyl | methyl | methyl | H |
| 2-22-374 | N-tolyl | N-tolyl | b1 | b1 | H |
| 2-22-375 | N-tolyl | N-tolyl | b2 | b2 | H |
| 2-22-376 | N-tolyl | N-tolyl | b3 | b3 | H |
| 2-22-377 | N-tolyl | N-tolyl | H | H | H |
| 2-22-378 | N-tolyl | N-tolyl | t-bu | t-bu | H |
| 2-22-379 | N-tolyl | N-tolyl | methyl | methyl | H |
| 2-22-380 | N-tolyl | N-tolyl | b1 | b1 | H |
| 2-22-381 | N-tolyl | N-tolyl | b2 | b2 | H |
| 2-22-382 | N-tolyl | N-tolyl | b3 | b3 | H |
| 2-22-383 | N-tolyl | N-tolyl | H | H | H |
| 2-22-384 | N-tolyl | N-tolyl | t-bu | t-bu | H |
| 2-22-385 | N-tolyl | N-tolyl | methyl | methyl | H |
| 2-22-386 | N-tolyl | N-tolyl | b1 | b1 | H |
| 2-22-387 | N-tolyl | N-tolyl | b2 | b2 | H |
| 2-22-388 | N-tolyl | N-tolyl | b3 | b3 | H |
| 2-22-389 | N-tolyl | N-tolyl | H | H | H |
| 2-22-390 | N-tolyl | N-tolyl | t-bu | t-bu | H |
| 2-22-391 | N-tolyl | N-tolyl | methyl | methyl | H |
| 2-22-392 | N-tolyl | N-tolyl | b1 | b1 | H |
| 2-22-393 | N-tolyl | N-tolyl | b2 | b2 | H |
| 2-22-394 | N-tolyl | N-tolyl | b3 | b3 | H |
| 2-22-395 | N-tolyl | N-tolyl | H | H | H |
| 2-22-396 | N-tolyl | N-tolyl | t-bu | t-bu | H |
| 2-22-397 | N-tolyl | N-tolyl | methyl | methyl | H |
| 2-22-398 | N-tolyl | N-tolyl | b1 | b1 | H |
| 2-22-399 | N-tolyl | N-tolyl | b2 | b2 | H |
| 2-22-400 | N-tolyl | N-tolyl | b3 | b3 | H |
| 2-22-401 | N-tolyl | N-tolyl | H | H | H |
| 2-22-402 | N-tolyl | N-tolyl | t-bu | t-bu | H |
| 2-22-403 | N-tolyl | N-tolyl | methyl | methyl | H |
| 2-22-404 | N-tolyl | N-tolyl | b1 | b1 | H |
| 2-22-405 | N-tolyl | N-tolyl | b2 | b2 | H |
| 2-22-406 | N-tolyl | N-tolyl | b3 | b3 | H |
| 2-22-407 | N-tolyl | N-tolyl | H | H | H |
| 2-22-408 | N-tolyl | N-tolyl | t-bu | t-bu | H |
| 2-22-409 | N-tolyl | N-tolyl | methyl | methyl | H |
| 2-22-410 | N-tolyl | N-tolyl | b1 | b1 | H |
| 2-22-411 | N-tolyl | N-tolyl | b2 | b2 | H |
| 2-22-412 | N-tolyl | N-tolyl | b3 | b3 | H |
| 2-22-413 | N-tolyl | N-tolyl | H | H | H |
| 2-22-414 | N-tolyl | N-tolyl | t-bu | t-bu | H |
| 2-22-415 | N-tolyl | N-tolyl | methyl | methyl | H |
| 2-22-416 | N-tolyl | N-tolyl | b1 | b1 | H |
| 2-22-417 | N-tolyl | N-tolyl | b2 | b2 | H |
| 2-22-418 | N-tolyl | N-tolyl | b3 | b3 | H |
| 2-22-419 | N-tolyl | N-tolyl | H | H | H |
| 2-22-420 | N-tolyl | N-tolyl | t-bu | t-bu | H |
| 2-22-421 | N-tolyl | N-tolyl | methyl | methyl | H |
| 2-22-422 | N-tolyl | N-tolyl | b1 | b1 | H |
| 2-22-423 | N-tolyl | N-tolyl | b2 | b2 | H |
| 2-22-424 | N-tolyl | N-tolyl | b3 | b3 | H |
| 2-22-425 | N-tolyl | N-tolyl | H | H | H |
| 2-22-426 | N-tolyl | N-tolyl | t-bu | t-bu | H |
| 2-22-427 | N-tolyl | N-tolyl | methyl | methyl | H |
| 2-22-428 | N-tolyl | N-tolyl | b1 | b1 | H |
| 2-22-429 | N-tolyl | N-tolyl | b2 | b2 | H |
| 2-22-430 | N-tolyl | N-tolyl | b3 | b3 | H |
| 2-22-431 | N-tolyl | N-tolyl | H | H | H |
| 2-22-432 | N-tolyl | N-tolyl | t-bu | t-bu | H |
| 2-22-433 | N-tolyl | N-tolyl | methyl | methyl | H |
| 2-22-434 | N-tolyl | N-tolyl | b1 | b1 | H |
| 2-22-435 | N-tolyl | N-tolyl | b2 | b2 | H |
| 2-22-436 | N-tolyl | N-tolyl | b3 | b3 | H |
| 2-22-437 | N-tolyl | N-tolyl | H | H | H |
| 2-22-438 | N-tolyl | N-tolyl | t-bu | t-bu | H |
| 2-22-439 | N-tolyl | N-tolyl | methyl | methyl | H |
| 2-22-440 | N-tolyl | N-tolyl | b1 | b1 | H |
| 2-22-441 | N-tolyl | N-tolyl | b2 | b2 | H |
| 2-22-442 | N-tolyl | N-tolyl | b3 | b3 | H |
| 2-22-443 | N-tolyl | N-tolyl | H | H | H |
| 2-22-444 | N-tolyl | N-tolyl | t-bu | t-bu | H |
| 2-22-445 | N-tolyl | N-tolyl | methyl | methyl | H |
| 2-22-446 | N-tolyl | N-tolyl | b1 | b1 | H |
| 2-22-447 | N-tolyl | N-tolyl | b2 | b2 | H |
| 2-22-448 | N-tolyl | N-tolyl | b3 | b3 | H |
| 2-22-449 | N-tolyl | N-tolyl | H | H | H |
| 2-22-450 | N-tolyl | N-tolyl | t-bu | t-bu | H |
| 2-22-451 | N-tolyl | N-tolyl | methyl | methyl | H |
| 2-22-452 | N-tolyl | N-tolyl | b1 | b1 | H |
| 2-22-453 | N-tolyl | N-tolyl | b2 | b2 | H |
| 2-22-454 | N-tolyl | N-tolyl | b3 | b3 | H |
| 2-22-455 | N-tolyl | N-tolyl | H | H | a1 |
| 2-22-456 | N-tolyl | N-tolyl | t-bu | t-bu | a1 |
| 2-22-457 | N-tolyl | N-tolyl | methyl | methyl | a1 |
| 2-22-458 | N-tolyl | N-tolyl | b1 | b1 | a1 |
| 2-22-459 | N-tolyl | N-tolyl | b2 | b2 | a1 |
| 2-22-460 | N-tolyl | N-tolyl | b3 | b3 | a1 |
| 2-22-461 | N-tolyl | N-tolyl | H | H | a2 |
| 2-22-462 | N-tolyl | N-tolyl | t-bu | t-bu | a2 |
| 2-22-463 | N-tolyl | N-tolyl | methyl | methyl | a2 |
| 2-22-464 | N-tolyl | N-tolyl | b1 | b1 | a2 |
| 2-22-465 | N-tolyl | N-tolyl | b2 | b2 | a2 |
| 2-22-466 | N-tolyl | N-tolyl | b3 | b3 | a2 |
| 2-22-467 | N-tolyl | N-tolyl | H | H | a3 |
| 2-22-468 | N-tolyl | N-tolyl | t-bu | t-bu | a3 |
| 2-22-469 | N-tolyl | N-tolyl | methyl | methyl | a3 |
| 2-22-470 | N-tolyl | N-tolyl | b1 | b1 | a3 |
| 2-22-471 | N-tolyl | N-tolyl | b2 | b2 | a3 |
| 2-22-472 | N-tolyl | N-tolyl | b3 | b3 | a3 |
| 2-22-473 | N-tolyl | N-tolyl | H | H | b1 |
| 2-22-474 | N-tolyl | N-tolyl | t-bu | t-bu | b1 |
| 2-22-475 | N-tolyl | N-tolyl | methyl | methyl | b1 |
| 2-22-476 | N-tolyl | N-tolyl | b1 | b1 | b1 |
| 2-22-477 | N-tolyl | N-tolyl | b2 | b2 | b1 |
| 2-22-478 | N-tolyl | N-tolyl | b3 | b3 | b1 |
| 2-22-479 | N-tolyl | N-tolyl | H | H | b2 |
| 2-22-480 | N-tolyl | N-tolyl | t-bu | t-bu | b2 |
| 2-22-481 | N-tolyl | N-tolyl | methyl | methyl | b2 |
| 2-22-482 | N-tolyl | N-tolyl | b1 | b1 | b2 |
| 2-22-483 | N-tolyl | N-tolyl | b2 | b2 | b2 |
| 2-22-484 | N-tolyl | N-tolyl | b3 | b3 | b2 |
| 2-22-485 | N-tolyl | N-tolyl | H | H | b3 |
| 2-22-486 | N-tolyl | N-tolyl | t-bu | t-bu | b3 |
| 2-22-487 | N-tolyl | N-tolyl | methyl | methyl | b3 |
| 2-22-488 | N-tolyl | N-tolyl | b1 | b1 | b3 |
| 2-22-489 | N-tolyl | N-tolyl | b2 | b2 | b3 |
| 2-22-490 | N-tolyl | N-tolyl | b3 | b3 | b3 |
| 2-22-491 | N-tolyl | N-tolyl | H | H | b5 |
| 2-22-492 | N-tolyl | N-tolyl | t-bu | t-bu | b5 |
| 2-22-493 | N-tolyl | N-tolyl | methyl | methyl | b5 |
| 2-22-494 | N-tolyl | N-tolyl | b1 | b1 | b5 |
| 2-22-495 | N-tolyl | N-tolyl | b2 | b2 | b5 |
| 2-22-496 | N-tolyl | N-tolyl | b3 | b3 | b5 |
| 2-22-497 | N-tolyl | N-tolyl | H | H | b6 |
| 2-22-498 | N-tolyl | N-tolyl | t-bu | t-bu | b6 |
| 2-22-499 | N-tolyl | N-tolyl | methyl | methyl | b6 |
| 2-22-500 | N-tolyl | N-tolyl | b1 | b1 | b6 |
| 2-22-501 | N-tolyl | N-tolyl | b2 | b2 | b6 |
| 2-22-502 | N-tolyl | N-tolyl | b3 | b3 | b6 |
| 2-22-503 | N-tolyl | N-tolyl | H | H | b7 |
| 2-22-504 | N-tolyl | N-tolyl | t-bu | t-bu | b7 |
| 2-22-505 | N-tolyl | N-tolyl | methyl | methyl | b7 |
| 2-22-506 | N-tolyl | N-tolyl | b1 | b1 | b7 |
| 2-22-507 | N-tolyl | N-tolyl | b2 | b2 | b7 |
| 2-22-508 | N-tolyl | N-tolyl | b3 | b3 | b7 |
| 2-22-509 | N-Biph | N-Biph | H | H | H |
| 2-22-510 | N-Biph | N-Biph | t-bu | t-bu | H |
| 2-22-511 | N-Biph | N-Biph | methyl | methyl | H |
| 2-22-512 | N-Biph | N-Biph | t-bu | t-bu | t-bu |
| 2-22-513 | N-Biph | N-Biph | methyl | methyl | methyl |
| 2-22-514 | N-Biph | N-Biph | b1 | b1 | H |
| 2-22-515 | N-Biph | N-Biph | b2 | b2 | H |
| 2-22-516 | N-Biph | N-Biph | b3 | b3 | H |
| 2-22-517 | N-Biph | N-Biph | H | H | a1 |
| 2-22-518 | N-Biph | N-Biph | t-bu | t-bu | a1 |
| 2-22-519 | N-Biph | N-Biph | methyl | methyl | a1 |
| 2-22-520 | N-Biph | N-Biph | b1 | b1 | a1 |
| 2-22-521 | N-Biph | N-Biph | b2 | b2 | a1 |
| 2-22-522 | N-Biph | N-Biph | b3 | b3 | a1 |
| 2-22-523 | N-Biph | N-Biph | H | H | a2 |
| 2-22-524 | N-Biph | N-Biph | t-bu | t-bu | a2 |
| 2-22-525 | N-Biph | N-Biph | methyl | methyl | a2 |
| 2-22-526 | N-Biph | N-Biph | b1 | b1 | a2 |
| 2-22-527 | N-Biph | N-Biph | b2 | b2 | a2 |
| 2-22-528 | N-Biph | N-Biph | b3 | b3 | a2 |
| 2-22-529 | N-Biph | N-Biph | H | H | a3 |
| 2-22-530 | N-Biph | N-Biph | t-bu | t-bu | a3 |
| 2-22-531 | N-Biph | N-Biph | methyl | methyl | a3 |
| 2-22-532 | N-Biph | N-Biph | b1 | b1 | a3 |
| 2-22-533 | N-Biph | N-Biph | b2 | b2 | a3 |
| 2-22-534 | N-Biph | N-Biph | b3 | b3 | a3 |
| 2-22-535 | N-Biph | N-Biph | H | H | a4 |
| 2-22-536 | N-Biph | N-Biph | t-bu | t-bu | a4 |
| 2-22-537 | N-Biph | N-Biph | methyl | methyl | a4 |
| 2-22-538 | N-Biph | N-Biph | b1 | b1 | a4 |
| 2-22-539 | N-Biph | N-Biph | b2 | b2 | a4 |
| 2-22-540 | N-Biph | N-Biph | b3 | b3 | a4 |
| 2-22-541 | N-Biph | N-Biph | H | H | a5 |
| 2-22-542 | N-Biph | N-Biph | t-bu | t-bu | a5 |
| 2-22-543 | N-Biph | N-Biph | methyl | methyl | a5 |
| 2-22-544 | N-Biph | N-Biph | b1 | b1 | a5 |
| 2-22-545 | N-Biph | N-Biph | b2 | b2 | a5 |
| 2-22-546 | N-Biph | N-Biph | b3 | b3 | a5 |
| 2-22-547 | N-Biph | N-Biph | H | H | a6 |
| 2-22-548 | N-Biph | N-Biph | t-bu | t-bu | a6 |
| 2-22-549 | N-Biph | N-Biph | methyl | methyl | a6 |
| 2-22-550 | N-Biph | N-Biph | b1 | b1 | a6 |
| 2-22-551 | N-Biph | N-Biph | b2 | b2 | a6 |
| 2-22-552 | N-Biph | N-Biph | b3 | b3 | a6 |
| 2-22-553 | N-Biph | N-Biph | H | H | b1 |
| 2-22-554 | N-Biph | N-Biph | t-bu | t-bu | b1 |
| 2-22-555 | N-Biph | N-Biph | methyl | methyl | b1 |
| 2-22-556 | N-Biph | N-Biph | b1 | b1 | b1 |
| 2-22-557 | N-Biph | N-Biph | b2 | b2 | b1 |
| 2-22-558 | N-Biph | N-Biph | b3 | b3 | b1 |
| 2-22-559 | N-Biph | N-Biph | H | H | b2 |
| 2-22-560 | N-Biph | N-Biph | t-bu | t-bu | b2 |
| 2-22-561 | N-Biph | N-Biph | methyl | methyl | b2 |
| 2-22-562 | N-Biph | N-Biph | b1 | b1 | b2 |
| 2-22-563 | N-Biph | N-Biph | b2 | b2 | b2 |
| 2-22-564 | N-Biph | N-Biph | b3 | b3 | b2 |
| 2-22-565 | N-Biph | N-Biph | H | H | b3 |
| 2-22-566 | N-Biph | N-Biph | t-bu | t-bu | b3 |
| 2-22-567 | N-Biph | N-Biph | methyl | methyl | b3 |
| 2-22-568 | N-Biph | N-Biph | b1 | b1 | b3 |
| 2-22-569 | N-Biph | N-Biph | b2 | b2 | b3 |
| 2-22-570 | N-Biph | N-Biph | b3 | b3 | b3 |
| 2-22-571 | N-Biph | N-Biph | H | H | b4 |
| 2-22-572 | N-Biph | N-Biph | t-bu | t-bu | b4 |
| 2-22-573 | N-Biph | N-Biph | methyl | methyl | b4 |
| 2-22-574 | N-Biph | N-Biph | b1 | b1 | b4 |
| 2-22-575 | N-Biph | N-Biph | b2 | b2 | b4 |
| 2-22-576 | N-Biph | N-Biph | b3 | b3 | b4 |
| 2-22-577 | N-Biph | N-Biph | H | H | b5 |
| 2-22-578 | N-Biph | N-Biph | t-bu | t-bu | b5 |
| 2-22-579 | N-Biph | N-Biph | methyl | methyl | b5 |
| 2-22-580 | N-Biph | N-Biph | b1 | b1 | b5 |
| 2-22-581 | N-Biph | N-Biph | b2 | b2 | b5 |
| 2-22-582 | N-Biph | N-Biph | b3 | b3 | b5 |
| 2-22-583 | N-Biph | N-Biph | H | H | b6 |
| 2-22-584 | N-Biph | N-Biph | t-bu | t-bu | b6 |
| 2-22-585 | N-Biph | N-Biph | methyl | methyl | b6 |
| 2-22-586 | N-Biph | N-Biph | b1 | b1 | b6 |
| 2-22-587 | N-Biph | N-Biph | b2 | b2 | b6 |
| 2-22-588 | N-Biph | N-Biph | b3 | b3 | b6 |
| 2-22-589 | N-Biph | N-Biph | H | H | b7 |
| 2-22-590 | N-Biph | N-Biph | t-bu | t-bu | b7 |
| 2-22-591 | N-Biph | N-Biph | methyl | methyl | b7 |
| 2-22-592 | N-Biph | N-Biph | b1 | b1 | b7 |
| 2-22-593 | N-Biph | N-Biph | b2 | b2 | b7 |
| 2-22-594 | N-Biph | N-Biph | b3 | b3 | b7 |
| 2-22-595 | N-Biph | N-Biph | H | H | b8 |
| 2-22-596 | N-Biph | N-Biph | t-bu | t-bu | b8 |
| 2-22-597 | N-Biph | N-Biph | methyl | methyl | b8 |
| 2-22-598 | N-Biph | N-Biph | b1 | b1 | b8 |
| 2-22-599 | N-Biph | N-Biph | b2 | b2 | b8 |
| 2-22-600 | N-Biph | N-Biph | b3 | b3 | b8 |
| 2-22-601 | N-Biph | N-Biph | H | H | b9 |
| 2-22-602 | N-Biph | N-Biph | t-bu | t-bu | b9 |
| 2-22-603 | N-Biph | N-Biph | methyl | methyl | b9 |
| 2-22-604 | N-Biph | N-Biph | b1 | b1 | b9 |
| 2-22-605 | N-Biph | N-Biph | b2 | b2 | b9 |
| 2-22-606 | N-Biph | N-Biph | b3 | b3 | b9 |
| 2-22-607 | N-Biph | N-Biph | H | H | b10 |
| 2-22-608 | N-Biph | N-Biph | t-bu | t-bu | b10 |
| 2-22-609 | N-Biph | N-Biph | methyl | methyl | b10 |
| 2-22-610 | N-Biph | N-Biph | b1 | b1 | b10 |
| 2-22-611 | N-Biph | N-Biph | b2 | b2 | b10 |
| 2-22-612 | N-Biph | N-Biph | b3 | b3 | b10 |
| 2-22-613 | N-Biph | N-Biph | H | H | H |
| 2-22-614 | N-Biph | N-Biph | t-bu | t-bu | H |
| 2-22-615 | N-Biph | N-Biph | methyl | methyl | H |
| 2-22-616 | N-Biph | N-Biph | b1 | b1 | H |
| 2-22-617 | N-Biph | N-Biph | b2 | b2 | H |
| 2-22-618 | N-Biph | N-Biph | b3 | b3 | H |
| 2-22-619 | N-Biph | N-Biph | H | H | H |
| 2-22-620 | N-Biph | N-Biph | t-bu | t-bu | H |
| 2-22-621 | N-Biph | N-Biph | methyl | methyl | H |
| 2-22-622 | N-Biph | N-Biph | b1 | b1 | H |
| 2-22-623 | N-Biph | N-Biph | b2 | b2 | H |
| 2-22-624 | N-Biph | N-Biph | b3 | b3 | H |
| 2-22-625 | N-Biph | N-Biph | H | H | H |
| 2-22-626 | N-Biph | N-Biph | t-bu | t-bu | H |
| 2-22-627 | N-Biph | N-Biph | methyl | methyl | H |
| 2-22-628 | N-Biph | N-Biph | b1 | b1 | H |
| 2-22-629 | N-Biph | N-Biph | b2 | b2 | H |
| 2-22-630 | N-Biph | N-Biph | b3 | b3 | H |
| 2-22-631 | N-Biph | N-Biph | H | H | H |
| 2-22-632 | N-Biph | N-Biph | t-bu | t-bu | H |
| 2-22-633 | N-Biph | N-Biph | methyl | methyl | H |
| 2-22-634 | N-Biph | N-Biph | b1 | b1 | H |
| 2-22-635 | N-Biph | N-Biph | b2 | b2 | H |
| 2-22-636 | N-Biph | N-Biph | b3 | b3 | H |
| 2-22-637 | N-Biph | N-Biph | H | H | H |
| 2-22-638 | N-Biph | N-Biph | t-bu | t-bu | H |
| 2-22-639 | N-Biph | N-Biph | methyl | methyl | H |
| 2-22-640 | N-Biph | N-Biph | b1 | b1 | H |
| 2-22-641 | N-Biph | N-Biph | b2 | b2 | H |
| 2-22-642 | N-Biph | N-Biph | b3 | b3 | H |
| 2-22-643 | N-Biph | N-Biph | H | H | H |
| 2-22-644 | N-Biph | N-Biph | t-bu | t-bu | H |
| 2-22-645 | N-Biph | N-Biph | methyl | methyl | H |
| 2-22-646 | N-Biph | N-Biph | b1 | b1 | H |
| 2-22-647 | N-Biph | N-Biph | b2 | b2 | H |
| 2-22-648 | N-Biph | N-Biph | b3 | b3 | H |
| 2-22-649 | N-Biph | N-Biph | H | H | H |
| 2-22-650 | N-Biph | N-Biph | t-bu | t-bu | H |
| 2-22-651 | N-Biph | N-Biph | methyl | methyl | H |
| 2-22-652 | N-Biph | N-Biph | b1 | b1 | H |
| 2-22-653 | N-Biph | N-Biph | b2 | b2 | H |
| 2-22-654 | N-Biph | N-Biph | b3 | b3 | H |
| 2-22-655 | N-Biph | N-Biph | H | H | H |
| 2-22-656 | N-Biph | N-Biph | t-bu | t-bu | H |
| 2-22-657 | N-Biph | N-Biph | methyl | methyl | H |
| 2-22-658 | N-Biph | N-Biph | b1 | b1 | H |
| 2-22-659 | N-Biph | N-Biph | b2 | b2 | H |
| 2-22-660 | N-Biph | N-Biph | b3 | b3 | H |
| 2-22-661 | N-Biph | N-Biph | H | H | H |
| 2-22-662 | N-Biph | N-Biph | t-bu | t-bu | H |
| 2-22-663 | N-Biph | N-Biph | methyl | methyl | H |
| 2-22-664 | N-Biph | N-Biph | b1 | b1 | H |
| 2-22-665 | N-Biph | N-Biph | b2 | b2 | H |
| 2-22-666 | N-Biph | N-Biph | b3 | b3 | H |
| 2-22-667 | N-Biph | N-Biph | H | H | H |
| 2-22-668 | N-Biph | N-Biph | t-bu | t-bu | H |
| 2-22-669 | N-Biph | N-Biph | methyl | methyl | H |
| 2-22-670 | N-Biph | N-Biph | b1 | b1 | H |
| 2-22-671 | N-Biph | N-Biph | b2 | b2 | H |
| 2-22-672 | N-Biph | N-Biph | b3 | b3 | H |
| 2-22-673 | N-Biph | N-Biph | H | H | H |
| 2-22-674 | N-Biph | N-Biph | t-bu | t-bu | H |
| 2-22-675 | N-Biph | N-Biph | methyl | methyl | H |
| 2-22-676 | N-Biph | N-Biph | b1 | b1 | H |
| 2-22-677 | N-Biph | N-Biph | b2 | b2 | H |
| 2-22-678 | N-Biph | N-Biph | b3 | b3 | H |
| 2-22-679 | N-Biph | N-Biph | H | H | H |
| 2-22-680 | N-Biph | N-Biph | t-bu | t-bu | H |
| 2-22-681 | N-Biph | N-Biph | methyl | methyl | H |
| 2-22-682 | N-Biph | N-Biph | b1 | b1 | H |
| 2-22-683 | N-Biph | N-Biph | b2 | b2 | H |
| 2-22-684 | N-Biph | N-Biph | b3 | b3 | H |
| 2-22-685 | N-Biph | N-Biph | H | H | H |
| 2-22-686 | N-Biph | N-Biph | t-bu | t-bu | H |
| 2-22-687 | N-Biph | N-Biph | methyl | methyl | H |
| 2-22-688 | N-Biph | N-Biph | b1 | b1 | H |
| 2-22-689 | N-Biph | N-Biph | b2 | b2 | H |
| 2-22-690 | N-Biph | N-Biph | b3 | b3 | H |
| 2-22-691 | N-Biph | N-Biph | H | H | H |
| 2-22-692 | N-Biph | N-Biph | t-bu | t-bu | H |
| 2-22-693 | N-Biph | N-Biph | methyl | methyl | H |
| 2-22-694 | N-Biph | N-Biph | b1 | b1 | H |
| 2-22-695 | N-Biph | N-Biph | b2 | b2 | H |
| 2-22-696 | N-Biph | N-Biph | b3 | b3 | H |
| 2-22-697 | N-Biph | N-Biph | H | H | H |
| 2-22-698 | N-Biph | N-Biph | t-bu | t-bu | H |
| 2-22-699 | N-Biph | N-Biph | methyl | methyl | H |
| 2-22-700 | N-Biph | N-Biph | b1 | b1 | H |
| 2-22-701 | N-Biph | N-Biph | b2 | b2 | H |
| 2-22-702 | N-Biph | N-Biph | b3 | b3 | H |
| 2-22-703 | N-Biph | N-Biph | H | H | H |
| 2-22-704 | N-Biph | N-Biph | t-bu | t-bu | H |
| 2-22-705 | N-Biph | N-Biph | methyl | methyl | H |
| 2-22-706 | N-Biph | N-Biph | b1 | b1 | H |
| 2-22-707 | N-Biph | N-Biph | b2 | b2 | H |
| 2-22-708 | N-Biph | N-Biph | b3 | b3 | H |
| 2-22-709 | N-Biph | N-Biph | H | H | a1 |
| 2-22-710 | N-Biph | N-Biph | t-bu | t-bu | a1 |
| 2-22-711 | N-Biph | N-Biph | methyl | methyl | a1 |
| 2-22-712 | N-Biph | N-Biph | b1 | b1 | a1 |
| 2-22-713 | N-Biph | N-Biph | b2 | b2 | a1 |
| 2-22-714 | N-Biph | N-Biph | b3 | b3 | a1 |
| 2-22-715 | N-Biph | N-Biph | H | H | a2 |
| 2-22-716 | N-Biph | N-Biph | t-bu | t-bu | a2 |
| 2-22-717 | N-Biph | N-Biph | methyl | methyl | a2 |
| 2-22-718 | N-Biph | N-Biph | b1 | b1 | a2 |
| 2-22-719 | N-Biph | N-Biph | b2 | b2 | a2 |
| 2-22-720 | N-Biph | N-Biph | b3 | b3 | a2 |
| 2-22-721 | N-Biph | N-Biph | H | H | a3 |
| 2-22-722 | N-Biph | N-Biph | t-bu | t-bu | a3 |
| 2-22-723 | N-Biph | N-Biph | methyl | methyl | a3 |
| 2-22-724 | N-Biph | N-Biph | b1 | b1 | a3 |
| 2-22-725 | N-Biph | N-Biph | b2 | b2 | a3 |
| 2-22-726 | N-Biph | N-Biph | b3 | b3 | a3 |
| 2-22-727 | N-Biph | N-Biph | H | H | b1 |
| 2-22-728 | N-Biph | N-Biph | t-bu | t-bu | b1 |
| 2-22-729 | N-Biph | N-Biph | methyl | methyl | b1 |
| 2-22-730 | N-Biph | N-Biph | b1 | b1 | b1 |
| 2-22-731 | N-Biph | N-Biph | b2 | b2 | b1 |
| 2-22-732 | N-Biph | N-Biph | b3 | b3 | b1 |
| 2-22-733 | N-Biph | N-Biph | H | H | b2 |
| 2-22-734 | N-Biph | N-Biph | t-bu | t-bu | b2 |
| 2-22-735 | N-Biph | N-Biph | methyl | methyl | b2 |
| 2-22-736 | N-Biph | N-Biph | b1 | b1 | b2 |
| 2-22-737 | N-Biph | N-Biph | b2 | b2 | b2 |
| 2-22-738 | N-Biph | N-Biph | b3 | b3 | b2 |
| 2-22-739 | N-Biph | N-Biph | H | H | b3 |
| 2-22-740 | N-Biph | N-Biph | t-bu | t-bu | b3 |
| 2-22-741 | N-Biph | N-Biph | methyl | methyl | b3 |
| 2-22-742 | N-Biph | N-Biph | b1 | b1 | b3 |
| 2-22-743 | N-Biph | N-Biph | b2 | b2 | b3 |
| 2-22-744 | N-Biph | N-Biph | b3 | b3 | b3 |
| 2-22-745 | N-Biph | N-Biph | H | H | b5 |
| 2-22-746 | N-Biph | N-Biph | t-bu | t-bu | b5 |
| 2-22-747 | N-Biph | N-Biph | methyl | methyl | b5 |
| 2-22-748 | N-Biph | N-Biph | b1 | b1 | b5 |
| 2-22-749 | N-Biph | N-Biph | b2 | b2 | b5 |
| 2-22-750 | N-Biph | N-Biph | b3 | b3 | b5 |
| 2-22-751 | N-Biph | N-Biph | H | H | b6 |
| 2-22-752 | N-Biph | N-Biph | t-bu | t-bu | b6 |
| 2-22-753 | N-Biph | N-Biph | methyl | methyl | b6 |
| 2-22-754 | N-Biph | N-Biph | b1 | b1 | b6 |
| 2-22-755 | N-Biph | N-Biph | b2 | b2 | b6 |
| 2-22-756 | N-Biph | N-Biph | b3 | b3 | b6 |
| 2-22-757 | N-Biph | N-Biph | H | H | b7 |
| 2-22-758 | N-Biph | N-Biph | t-bu | t-bu | b7 |
| 2-22-759 | N-Biph | N-Biph | methyl | methyl | b7 |
| 2-22-760 | N-Biph | N-Biph | b1 | b1 | b7 |
| 2-22-761 | N-Biph | N-Biph | b2 | b2 | b7 |
| 2-22-762 | N-Biph | N-Biph | b3 | b3 | b7 |
| 2-22-763 | O | N-Ph | H | H | H |
| 2-22-764 | O | N-Ph | t-bu | t-bu | H |
| 2-22-765 | O | N-Ph | methyl | methyl | H |
| 2-22-766 | O | N-Ph | t-bu | t-bu | t-bu |
| 2-22-767 | O | N-Ph | methyl | methyl | methyl |
| 2-22-768 | O | N-Ph | b1 | b1 | H |
| 2-22-769 | O | N-Ph | b2 | b2 | H |
| 2-22-770 | O | N-Ph | b3 | b3 | H |
| 2-22-771 | O | N-Ph | H | H | a1 |
| 2-22-772 | O | N-Ph | t-bu | t-bu | a1 |
| 2-22-773 | O | N-Ph | methyl | methyl | a1 |
| 2-22-774 | O | N-Ph | b1 | b1 | a1 |
| 2-22-775 | O | N-Ph | b2 | b2 | a1 |
| 2-22-776 | O | N-Ph | b3 | b3 | a1 |
| 2-22-777 | O | N-Ph | H | H | a2 |
| 2-22-778 | O | N-Ph | t-bu | t-bu | a2 |
| 2-22-779 | O | N-Ph | methyl | methyl | a2 |
| 2-22-780 | O | N-Ph | b1 | b1 | a2 |
| 2-22-781 | O | N-Ph | b2 | b2 | a2 |
| 2-22-782 | O | N-Ph | b3 | b3 | a2 |
| 2-22-783 | O | N-Ph | H | H | a3 |
| 2-22-784 | O | N-Ph | t-bu | t-bu | a3 |
| 2-22-785 | O | N-Ph | methyl | methyl | a3 |
| 2-22-786 | O | N-Ph | b1 | b1 | a3 |
| 2-22-787 | O | N-Ph | b2 | b2 | a3 |
| 2-22-788 | O | N-Ph | b3 | b3 | a3 |
| 2-22-789 | O | N-Ph | H | H | a4 |
| 2-22-790 | O | N-Ph | t-bu | t-bu | a4 |
| 2-22-791 | O | N-Ph | methyl | methyl | a4 |
| 2-22-792 | O | N-Ph | b1 | b1 | a4 |
| 2-22-793 | O | N-Ph | b2 | b2 | a4 |
| 2-22-794 | O | N-Ph | b3 | b3 | a4 |
| 2-22-795 | O | N-Ph | H | H | a5 |
| 2-22-796 | O | N-Ph | t-bu | t-bu | a5 |
| 2-22-797 | O | N-Ph | methyl | methyl | a5 |
| 2-22-798 | O | N-Ph | b1 | b1 | a5 |
| 2-22-799 | O | N-Ph | b2 | b2 | a5 |
| 2-22-800 | O | N-Ph | b3 | b3 | a5 |
| 2-22-801 | O | N-Ph | H | H | a6 |
| 2-22-802 | O | N-Ph | t-bu | t-bu | a6 |
| 2-22-803 | O | N-Ph | methyl | methyl | a6 |
| 2-22-804 | O | N-Ph | b1 | b1 | a6 |
| 2-22-805 | O | N-Ph | b2 | b2 | a6 |
| 2-22-806 | O | N-Ph | b3 | b3 | a6 |
| 2-22-807 | O | N-Ph | H | H | b1 |
| 2-22-808 | O | N-Ph | t-bu | t-bu | b1 |
| 2-22-809 | O | N-Ph | methyl | methyl | b1 |
| 2-22-810 | O | N-Ph | b1 | b1 | b1 |
| 2-22-811 | O | N-Ph | b2 | b2 | b1 |
| 2-22-812 | O | N-Ph | b3 | b3 | b1 |
| 2-22-813 | O | N-Ph | H | H | b2 |
| 2-22-814 | O | N-Ph | t-bu | t-bu | b2 |
| 2-22-815 | O | N-Ph | methyl | methyl | b2 |
| 2-22-816 | O | N-Ph | b1 | b1 | b2 |
| 2-22-817 | O | N-Ph | b2 | b2 | b2 |
| 2-22-818 | O | N-Ph | b3 | b3 | b2 |
| 2-22-819 | O | N-Ph | H | H | b3 |
| 2-22-820 | O | N-Ph | t-bu | t-bu | b3 |
| 2-22-821 | O | N-Ph | methyl | methyl | b3 |
| 2-22-822 | O | N-Ph | b1 | b1 | b3 |
| 2-22-823 | O | N-Ph | b2 | b2 | b3 |
| 2-22-824 | O | N-Ph | b3 | b3 | b3 |
| 2-22-825 | O | N-Ph | H | H | b4 |
| 2-22-826 | O | N-Ph | t-bu | t-bu | b4 |
| 2-22-827 | O | N-Ph | methyl | methyl | b4 |
| 2-22-828 | O | N-Ph | b1 | b1 | b4 |
| 2-22-829 | O | N-Ph | b2 | b2 | b4 |
| 2-22-830 | O | N-Ph | b3 | b3 | b4 |
| 2-22-831 | O | N-Ph | H | H | b5 |
| 2-22-832 | O | N-Ph | t-bu | t-bu | b5 |
| 2-22-833 | O | N-Ph | methyl | methyl | b5 |
| 2-22-834 | O | N-Ph | b1 | b1 | b5 |
| 2-22-835 | O | N-Ph | b2 | b2 | b5 |
| 2-22-836 | O | N-Ph | b3 | b3 | b5 |
| 2-22-837 | O | N-Ph | H | H | b6 |
| 2-22-838 | O | N-Ph | t-bu | t-bu | b6 |
| 2-22-839 | O | N-Ph | methyl | methyl | b6 |
| 2-22-840 | O | N-Ph | b1 | b1 | b6 |
| 2-22-841 | O | N-Ph | b2 | b2 | b6 |
| 2-22-842 | O | N-Ph | b3 | b3 | b6 |
| 2-22-843 | O | N-Ph | H | H | b7 |
| 2-22-844 | O | N-Ph | t-bu | t-bu | b7 |
| 2-22-845 | O | N-Ph | methyl | methyl | b7 |
| 2-22-846 | O | N-Ph | b1 | b1 | b7 |
| 2-22-847 | O | N-Ph | b2 | b2 | b7 |
| 2-22-848 | O | N-Ph | b3 | b3 | b7 |
| 2-22-849 | O | N-Ph | H | H | b8 |
| 2-22-850 | O | N-Ph | t-bu | t-bu | b8 |
| 2-22-851 | O | N-Ph | methyl | methyl | b8 |
| 2-22-852 | O | N-Ph | b1 | b1 | b8 |
| 2-22-853 | O | N-Ph | b2 | b2 | b8 |
| 2-22-854 | O | N-Ph | b3 | b3 | b8 |
| 2-22-855 | O | N-Ph | H | H | b9 |
| 2-22-856 | O | N-Ph | t-bu | t-bu | b9 |
| 2-22-857 | O | N-Ph | methyl | methyl | b9 |
| 2-22-858 | O | N-Ph | b1 | b1 | b9 |
| 2-22-859 | O | N-Ph | b2 | b2 | b9 |
| 2-22-860 | O | N-Ph | b3 | b3 | b9 |
| 2-22-861 | O | N-Ph | H | H | b10 |
| 2-22-862 | O | N-Ph | t-bu | t-bu | b10 |
| 2-22-863 | O | N-Ph | methyl | methyl | b10 |
| 2-22-864 | O | N-Ph | b1 | b1 | b10 |
| 2-22-865 | O | N-Ph | b2 | b2 | b10 |
| 2-22-866 | O | N-Ph | b3 | b3 | b10 |
| 2-22-867 | O | N-Ph | H | H | H |
| 2-22-868 | O | N-Ph | t-bu | t-bu | H |
| 2-22-869 | O | N-Ph | methyl | methyl | H |
| 2-22-870 | O | N-Ph | b1 | b1 | H |
| 2-22-871 | O | N-Ph | b2 | b2 | H |
| 2-22-872 | O | N-Ph | b3 | b3 | H |
| 2-22-873 | O | N-Ph | H | H | H |
| 2-22-874 | O | N-Ph | t-bu | t-bu | H |
| 2-22-875 | O | N-Ph | methyl | methyl | H |
| 2-22-876 | O | N-Ph | b1 | b1 | H |
| 2-22-877 | O | N-Ph | b2 | b2 | H |
| 2-22-878 | O | N-Ph | b3 | b3 | H |
| 2-22-879 | O | N-Ph | H | H | H |
| 2-22-880 | O | N-Ph | t-bu | t-bu | H |
| 2-22-881 | O | N-Ph | methyl | methyl | H |
| 2-22-882 | O | N-Ph | b1 | b1 | H |
| 2-22-883 | O | N-Ph | b2 | b2 | H |
| 2-22-884 | O | N-Ph | b3 | b3 | H |
| 2-22-885 | O | N-Ph | H | H | H |
| 2-22-886 | O | N-Ph | t-bu | t-bu | H |
| 2-22-887 | O | N-Ph | methyl | methyl | H |
| 2-22-888 | O | N-Ph | b1 | b1 | H |
| 2-22-889 | O | N-Ph | b2 | b2 | H |
| 2-22-890 | O | N-Ph | b3 | b3 | H |
| 2-22-891 | O | N-Ph | H | H | H |
| 2-22-892 | O | N-Ph | t-bu | t-bu | H |
| 2-22-893 | O | N-Ph | methyl | methyl | H |
| 2-22-894 | O | N-Ph | b1 | b1 | H |
| 2-22-895 | O | N-Ph | b2 | b2 | H |
| 2-22-896 | O | N-Ph | b3 | b3 | H |
| 2-22-897 | O | N-Ph | H | H | H |
| 2-22-898 | O | N-Ph | t-bu | t-bu | H |
| 2-22-899 | O | N-Ph | methyl | methyl | H |
| 2-22-900 | O | N-Ph | b1 | b1 | H |
| 2-22-901 | O | N-Ph | b2 | b2 | H |
| 2-22-902 | O | N-Ph | b3 | b3 | H |
| 2-22-903 | O | N-Ph | H | H | H |
| 2-22-904 | O | N-Ph | t-bu | t-bu | H |
| 2-22-905 | O | N-Ph | methyl | methyl | H |
| 2-22-906 | O | N-Ph | b1 | b1 | H |
| 2-22-907 | O | N-Ph | b2 | b2 | H |
| 2-22-908 | O | N-Ph | b3 | b3 | H |
| 2-22-909 | O | N-Ph | H | H | H |
| 2-22-910 | O | N-Ph | t-bu | t-bu | H |
| 2-22-911 | O | N-Ph | methyl | methyl | H |
| 2-22-912 | O | N-Ph | b1 | b1 | H |
| 2-22-913 | O | N-Ph | b2 | b2 | H |
| 2-22-914 | O | N-Ph | b3 | b3 | H |
| 2-22-915 | O | N-Ph | H | H | H |
| 2-22-916 | O | N-Ph | t-bu | t-bu | H |
| 2-22-917 | O | N-Ph | methyl | methyl | H |
| 2-22-918 | O | N-Ph | b1 | b1 | H |
| 2-22-919 | O | N-Ph | b2 | b2 | H |
| 2-22-920 | O | N-Ph | b3 | b3 | H |
| 2-22-921 | O | N-Ph | H | H | H |
| 2-22-922 | O | N-Ph | t-bu | t-bu | H |
| 2-22-923 | O | N-Ph | methyl | methyl | H |
| 2-22-924 | O | N-Ph | b1 | b1 | H |
| 2-22-925 | O | N-Ph | b2 | b2 | H |
| 2-22-926 | O | N-Ph | b3 | b3 | H |
| 2-22-927 | O | N-Ph | H | H | H |
| 2-22-928 | O | N-Ph | t-bu | t-bu | H |
| 2-22-929 | O | N-Ph | methyl | methyl | H |
| 2-22-930 | O | N-Ph | b1 | b1 | H |
| 2-22-931 | O | N-Ph | b2 | b2 | H |
| 2-22-932 | O | N-Ph | b3 | b3 | H |
| 2-22-933 | O | N-Ph | H | H | H |
| 2-22-934 | O | N-Ph | t-bu | t-bu | H |
| 2-22-935 | O | N-Ph | methyl | methyl | H |
| 2-22-936 | O | N-Ph | b1 | b1 | H |
| 2-22-937 | O | N-Ph | b2 | b2 | H |
| 2-22-938 | O | N-Ph | b3 | b3 | H |
| 2-22-939 | O | N-Ph | H | H | H |
| 2-22-940 | O | N-Ph | t-bu | t-bu | H |
| 2-22-941 | O | N-Ph | methyl | methyl | H |
| 2-22-942 | O | N-Ph | b1 | b1 | H |
| 2-22-943 | O | N-Ph | b2 | b2 | H |
| 2-22-944 | O | N-Ph | b3 | b3 | H |
| 2-22-945 | O | N-Ph | H | H | H |
| 2-22-946 | O | N-Ph | t-bu | t-bu | H |
| 2-22-947 | O | N-Ph | methyl | methyl | H |
| 2-22-948 | O | N-Ph | b1 | b1 | H |
| 2-22-949 | O | N-Ph | b2 | b2 | H |
| 2-22-950 | O | N-Ph | b3 | b3 | H |
| 2-22-951 | O | N-Ph | H | H | H |
| 2-22-952 | O | N-Ph | t-bu | t-bu | H |
| 2-22-953 | O | N-Ph | methyl | methyl | H |
| 2-22-954 | O | N-Ph | b1 | b1 | H |
| 2-22-955 | O | N-Ph | b2 | b2 | H |
| 2-22-956 | O | N-Ph | b3 | b3 | H |
| 2-22-957 | O | N-Ph | H | H | H |
| 2-22-958 | O | N-Ph | t-bu | t-bu | H |
| 2-22-959 | O | N-Ph | methyl | methyl | H |
| 2-22-960 | O | N-Ph | b1 | b1 | H |
| 2-22-961 | O | N-Ph | b2 | b2 | H |
| 2-22-962 | O | N-Ph | b3 | b3 | H |
| 2-22-963 | O | N-Ph | H | H | a1 |
| 2-22-964 | O | N-Ph | t-bu | t-bu | a1 |
| 2-22-965 | O | N-Ph | methyl | methyl | a1 |
| 2-22-966 | O | N-Ph | b1 | b1 | a1 |
| 2-22-967 | O | N-Ph | b2 | b2 | a1 |
| 2-22-968 | O | N-Ph | b3 | b3 | a1 |
| 2-22-969 | O | N-Ph | H | H | a2 |
| 2-22-970 | O | N-Ph | t-bu | t-bu | a2 |
| 2-22-971 | O | N-Ph | methyl | methyl | a2 |
| 2-22-972 | O | N-Ph | b1 | b1 | a2 |
| 2-22-973 | O | N-Ph | b2 | b2 | a2 |
| 2-22-974 | O | N-Ph | b3 | b3 | a2 |
| 2-22-975 | O | N-Ph | H | H | a3 |
| 2-22-976 | O | N-Ph | t-bu | t-bu | a3 |
| 2-22-977 | O | N-Ph | methyl | methyl | a3 |
| 2-22-978 | O | N-Ph | b1 | b1 | a3 |
| 2-22-979 | O | N-Ph | b2 | b2 | a3 |
| 2-22-980 | O | N-Ph | b3 | b3 | a3 |
| 2-22-981 | O | N-Ph | H | H | b1 |
| 2-22-982 | O | N-Ph | t-bu | t-bu | b1 |
| 2-22-983 | O | N-Ph | methyl | methyl | b1 |
| 2-22-984 | O | N-Ph | b1 | b1 | b1 |
| 2-22-985 | O | N-Ph | b2 | b2 | b1 |
| 2-22-986 | O | N-Ph | b3 | b3 | b1 |
| 2-22-987 | O | N-Ph | H | H | b2 |
| 2-22-988 | O | N-Ph | t-bu | t-bu | b2 |
| 2-22-989 | O | N-Ph | methyl | methyl | b2 |
| 2-22-990 | O | N-Ph | b1 | b1 | b2 |
| 2-22-991 | O | N-Ph | b2 | b2 | b2 |
| 2-22-992 | O | N-Ph | b3 | b3 | b2 |
| 2-22-993 | O | N-Ph | H | H | b3 |
| 2-22-994 | O | N-Ph | t-bu | t-bu | b3 |
| 2-22-995 | O | N-Ph | methyl | methyl | b3 |
| 2-22-996 | O | N-Ph | b1 | b1 | b3 |
| 2-22-997 | O | N-Ph | b2 | b2 | b3 |
| 2-22-998 | O | N-Ph | b3 | b3 | b3 |
| 2-22-999 | O | N-Ph | H | H | b5 |
| 2-22-1000 | O | N-Ph | t-bu | t-bu | b5 |
| 2-22-1001 | O | N-Ph | methyl | methyl | b5 |
| 2-22-1002 | O | N-Ph | b1 | b1 | b5 |
| 2-22-1003 | O | N-Ph | b2 | b2 | b5 |
| 2-22-1004 | O | N-Ph | b3 | b3 | b5 |
| 2-22-1005 | O | N-Ph | H | H | b6 |
| 2-22-1006 | O | N-Ph | t-bu | t-bu | b6 |
| 2-22-1007 | O | N-Ph | methyl | methyl | b6 |
| 2-22-1008 | O | N-Ph | b1 | b1 | b6 |
| 2-22-1009 | O | N-Ph | b2 | b2 | b6 |
| 2-22-1010 | O | N-Ph | b3 | b3 | b6 |
| 2-22-1011 | O | N-Ph | H | H | b7 |
| 2-22-1012 | O | N-Ph | t-bu | t-bu | b7 |
| 2-22-1013 | O | N-Ph | methyl | methyl | b7 |
| 2-22-1014 | O | N-Ph | b1 | b1 | b7 |
| 2-22-1015 | O | N-Ph | b2 | b2 | b7 |
| 2-22-1016 | O | N-Ph | b3 | b3 | b7 |
| 2-22-1017 | S | N-Ph | H | H | H |
| 2-22-1018 | S | N-Ph | t-bu | t-bu | H |
| 2-22-1019 | S | N-Ph | methyl | methyl | H |
| 2-22-1020 | S | N-Ph | t-bu | t-bu | t-bu |
| 2-22-1021 | S | N-Ph | methyl | methyl | methyl |
| 2-22-1022 | S | N-Ph | b1 | b1 | H |
| 2-22-1023 | S | N-Ph | b2 | b2 | H |
| 2-22-1024 | S | N-Ph | b3 | b3 | H |
| 2-22-1025 | S | N-Ph | H | H | a1 |
| 2-22-1026 | S | N-Ph | t-bu | t-bu | a1 |
| 2-22-1027 | S | N-Ph | methyl | methyl | a1 |
| 2-22-1028 | S | N-Ph | b1 | b1 | a1 |
| 2-22-1029 | S | N-Ph | b2 | b2 | a1 |
| 2-22-1030 | S | N-Ph | b3 | b3 | a1 |
| 2-22-1031 | S | N-Ph | H | H | a2 |
| 2-22-1032 | S | N-Ph | t-bu | t-bu | a2 |
| 2-22-1033 | S | N-Ph | methyl | methyl | a2 |
| 2-22-1034 | S | N-Ph | b1 | b1 | a2 |
| 2-22-1035 | S | N-Ph | b2 | b2 | a2 |
| 2-22-1036 | S | N-Ph | b3 | b3 | a2 |
| 2-22-1037 | S | N-Ph | H | H | a3 |
| 2-22-1038 | S | N-Ph | t-bu | t-bu | a3 |
| 2-22-1039 | S | N-Ph | methyl | methyl | a3 |
| 2-22-1040 | S | N-Ph | b1 | b1 | a3 |
| 2-22-1041 | S | N-Ph | b2 | b2 | a3 |
| 2-22-1042 | S | N-Ph | b3 | b3 | a3 |
| 2-22-1043 | S | N-Ph | H | H | a4 |
| 2-22-1044 | S | N-Ph | t-bu | t-bu | a4 |
| 2-22-1045 | S | N-Ph | methyl | methyl | a4 |
| 2-22-1046 | S | N-Ph | b1 | b1 | a4 |
| 2-22-1047 | S | N-Ph | b2 | b2 | a4 |
| 2-22-1048 | S | N-Ph | b3 | b3 | a4 |
| 2-22-1049 | S | N-Ph | H | H | a5 |
| 2-22-1050 | S | N-Ph | t-bu | t-bu | a5 |
| 2-22-1051 | S | N-Ph | methyl | methyl | a5 |
| 2-22-1052 | S | N-Ph | b1 | b1 | a5 |
| 2-22-1053 | S | N-Ph | b2 | b2 | a5 |
| 2-22-1054 | S | N-Ph | b3 | b3 | a5 |
| 2-22-1055 | S | N-Ph | H | H | a6 |
| 2-22-1056 | S | N-Ph | t-bu | t-bu | a6 |
| 2-22-1057 | S | N-Ph | methyl | methyl | a6 |
| 2-22-1058 | S | N-Ph | b1 | b1 | a6 |
| 2-22-1059 | S | N-Ph | b2 | b2 | a6 |
| 2-22-1060 | S | N-Ph | b3 | b3 | a6 |
| 2-22-1061 | S | N-Ph | H | H | b1 |
| 2-22-1062 | S | N-Ph | t-bu | t-bu | b1 |
| 2-22-1063 | S | N-Ph | methyl | methyl | b1 |
| 2-22-1064 | S | N-Ph | b1 | b1 | b1 |
| 2-22-1065 | S | N-Ph | b2 | b2 | b1 |
| 2-22-1066 | S | N-Ph | b3 | b3 | b1 |
| 2-22-1067 | S | N-Ph | H | H | b2 |
| 2-22-1068 | S | N-Ph | t-bu | t-bu | b2 |
| 2-22-1069 | S | N-Ph | methyl | methyl | b2 |
| 2-22-1070 | S | N-Ph | b1 | b1 | b2 |
| 2-22-1071 | S | N-Ph | b2 | b2 | b2 |
| 2-22-1072 | S | N-Ph | b3 | b3 | b2 |
| 2-22-1073 | S | N-Ph | H | H | b3 |
| 2-22-1074 | S | N-Ph | t-bu | t-bu | b3 |
| 2-22-1075 | S | N-Ph | methyl | methyl | b3 |
| 2-22-1076 | S | N-Ph | b1 | b1 | b3 |
| 2-22-1077 | S | N-Ph | b2 | b2 | b3 |
| 2-22-1078 | S | N-Ph | b3 | b3 | b3 |
| 2-22-1079 | S | N-Ph | H | H | b4 |
| 2-22-1080 | S | N-Ph | t-bu | t-bu | b4 |
| 2-22-1081 | S | N-Ph | methyl | methyl | b4 |
| 2-22-1082 | S | N-Ph | b1 | b1 | b4 |
| 2-22-1083 | S | N-Ph | b2 | b2 | b4 |
| 2-22-1084 | S | N-Ph | b3 | b3 | b4 |
| 2-22-1085 | S | N-Ph | H | H | b5 |
| 2-22-1086 | S | N-Ph | t-bu | t-bu | b5 |
| 2-22-1087 | S | N-Ph | methyl | methyl | b5 |
| 2-22-1088 | S | N-Ph | b1 | b1 | b5 |
| 2-22-1089 | S | N-Ph | b2 | b2 | b5 |
| 2-22-1090 | S | N-Ph | b3 | b3 | b5 |
| 2-22-1091 | S | N-Ph | H | H | b6 |
| 2-22-1092 | S | N-Ph | t-bu | t-bu | b6 |
| 2-22-1093 | S | N-Ph | methyl | methyl | b6 |
| 2-22-1094 | S | N-Ph | b1 | b1 | b6 |
| 2-22-1095 | S | N-Ph | b2 | b2 | b6 |
| 2-22-1096 | S | N-Ph | b3 | b3 | b6 |
| 2-22-1097 | S | N-Ph | H | H | b7 |
| 2-22-1098 | S | N-Ph | t-bu | t-bu | b7 |
| 2-22-1099 | S | N-Ph | methyl | methyl | b7 |
| 2-22-1100 | S | N-Ph | b1 | b1 | b7 |
| 2-22-1101 | S | N-Ph | b2 | b2 | b7 |
| 2-22-1102 | S | N-Ph | b3 | b3 | b7 |
| 2-22-1103 | S | N-Ph | H | H | b8 |
| 2-22-1104 | S | N-Ph | t-bu | t-bu | b8 |
| 2-22-1105 | S | N-Ph | methyl | methyl | b8 |
| 2-22-1106 | S | N-Ph | b1 | b1 | b8 |
| 2-22-1107 | S | N-Ph | b2 | b2 | b8 |
| 2-22-1108 | S | N-Ph | b3 | b3 | b8 |
| 2-22-1109 | S | N-Ph | H | H | b9 |
| 2-22-1110 | S | N-Ph | t-bu | t-bu | b9 |
| 2-22-1111 | S | N-Ph | methyl | methyl | b9 |
| 2-22-1112 | S | N-Ph | b1 | b1 | b9 |
| 2-22-1113 | S | N-Ph | b2 | b2 | b9 |
| 2-22-1114 | S | N-Ph | b3 | b3 | b9 |
| 2-22-1115 | S | N-Ph | H | H | b10 |
| 2-22-1116 | S | N-Ph | t-bu | t-bu | b10 |
| 2-22-1117 | S | N-Ph | methyl | methyl | b10 |
| 2-22-1118 | S | N-Ph | b1 | b1 | b10 |
| 2-22-1119 | S | N-Ph | b2 | b2 | b10 |
| 2-22-1120 | S | N-Ph | b3 | b3 | b10 |
| 2-22-1121 | S | N-Ph | H | H | H |
| 2-22-1122 | S | N-Ph | t-bu | t-bu | H |
| 2-22-1123 | S | N-Ph | methyl | methyl | H |
| 2-22-1124 | S | N-Ph | b1 | b1 | H |
| 2-22-1125 | S | N-Ph | b2 | b2 | H |
| 2-22-1126 | S | N-Ph | b3 | b3 | H |
| 2-22-1127 | S | N-Ph | H | H | H |
| 2-22-1128 | S | N-Ph | t-bu | t-bu | H |
| 2-22-1129 | S | N-Ph | methyl | methyl | H |
| 2-22-1130 | S | N-Ph | b1 | b1 | H |
| 2-22-1131 | S | N-Ph | b2 | b2 | H |
| 2-22-1132 | S | N-Ph | b3 | b3 | H |
| 2-22-1133 | S | N-Ph | H | H | H |
| 2-22-1134 | S | N-Ph | t-bu | t-bu | H |
| 2-22-1135 | S | N-Ph | methyl | methyl | H |
| 2-22-1136 | S | N-Ph | b1 | b1 | H |
| 2-22-1137 | S | N-Ph | b2 | b2 | H |
| 2-22-1138 | S | N-Ph | b3 | b3 | H |
| 2-22-1139 | S | N-Ph | H | H | H |
| 2-22-1140 | S | N-Ph | t-bu | t-bu | H |
| 2-22-1141 | S | N-Ph | methyl | methyl | H |
| 2-22-1142 | S | N-Ph | b1 | b1 | H |
| 2-22-1143 | S | N-Ph | b2 | b2 | H |
| 2-22-1144 | S | N-Ph | b3 | b3 | H |
| 2-22-1145 | S | N-Ph | H | H | H |
| 2-22-1146 | S | N-Ph | t-bu | t-bu | H |
| 2-22-1147 | S | N-Ph | methyl | methyl | H |
| 2-22-1148 | S | N-Ph | b1 | b1 | H |
| 2-22-1149 | S | N-Ph | b2 | b2 | H |
| 2-22-1150 | S | N-Ph | b3 | b3 | H |
| 2-22-1151 | S | N-Ph | H | H | H |
| 2-22-1152 | S | N-Ph | t-bu | t-bu | H |
| 2-22-1153 | S | N-Ph | methyl | methyl | H |
| 2-22-1154 | S | N-Ph | b1 | b1 | H |
| 2-22-1155 | S | N-Ph | b2 | b2 | H |
| 2-22-1156 | S | N-Ph | b3 | b3 | H |
| 2-22-1157 | S | N-Ph | H | H | H |
| 2-22-1158 | S | N-Ph | t-bu | t-bu | H |
| 2-22-1159 | S | N-Ph | methyl | methyl | H |
| 2-22-1160 | S | N-Ph | b1 | b1 | H |
| 2-22-1161 | S | N-Ph | b2 | b2 | H |
| 2-22-1162 | S | N-Ph | b3 | b3 | H |
| 2-22-1163 | S | N-Ph | H | H | H |
| 2-22-1164 | S | N-Ph | t-bu | t-bu | H |
| 2-22-1165 | S | N-Ph | methyl | methyl | H |
| 2-22-1166 | S | N-Ph | b1 | b1 | H |
| 2-22-1167 | S | N-Ph | b2 | b2 | H |
| 2-22-1168 | S | N-Ph | b3 | b3 | H |
| 2-22-1169 | S | N-Ph | H | H | H |
| 2-22-1170 | S | N-Ph | t-bu | t-bu | H |
| 2-22-1171 | S | N-Ph | methyl | methyl | H |
| 2-22-1172 | S | N-Ph | b1 | b1 | H |
| 2-22-1173 | S | N-Ph | b2 | b2 | H |
| 2-22-1174 | S | N-Ph | b3 | b3 | H |
| 2-22-1175 | S | N-Ph | H | H | H |
| 2-22-1176 | S | N-Ph | t-bu | t-bu | H |
| 2-22-1177 | S | N-Ph | methyl | methyl | H |
| 2-22-1178 | S | N-Ph | b1 | b1 | H |
| 2-22-1179 | S | N-Ph | b2 | b2 | H |
| 2-22-1180 | S | N-Ph | b3 | b3 | H |
| 2-22-1181 | S | N-Ph | H | H | H |
| 2-22-1182 | S | N-Ph | t-bu | t-bu | H |
| 2-22-1183 | S | N-Ph | methyl | methyl | H |
| 2-22-1184 | S | N-Ph | b1 | b1 | H |
| 2-22-1185 | S | N-Ph | b2 | b2 | H |
| 2-22-1186 | S | N-Ph | b3 | b3 | H |
| 2-22-1187 | S | N-Ph | H | H | H |
| 2-22-1188 | S | N-Ph | t-bu | t-bu | H |
| 2-22-1189 | S | N-Ph | methyl | methyl | H |
| 2-22-1190 | S | N-Ph | b1 | b1 | H |
| 2-22-1191 | S | N-Ph | b2 | b2 | H |
| 2-22-1192 | S | N-Ph | b3 | b3 | H |
| 2-22-1193 | S | N-Ph | H | H | H |
| 2-22-1194 | S | N-Ph | t-bu | t-bu | H |
| 2-22-1195 | S | N-Ph | methyl | methyl | H |
| 2-22-1196 | S | N-Ph | b1 | b1 | H |
| 2-22-1197 | S | N-Ph | b2 | b2 | H |
| 2-22-1198 | S | N-Ph | b3 | b3 | H |
| 2-22-1199 | S | N-Ph | H | H | H |
| 2-22-1200 | S | N-Ph | t-bu | t-bu | H |
| 2-22-1201 | S | N-Ph | methyl | methyl | H |
| 2-22-1202 | S | N-Ph | b1 | b1 | H |
| 2-22-1203 | S | N-Ph | b2 | b2 | H |
| 2-22-1204 | S | N-Ph | b3 | b3 | H |
| 2-22-1205 | S | N-Ph | H | H | H |
| 2-22-1206 | S | N-Ph | t-bu | t-bu | H |
| 2-22-1207 | S | N-Ph | methyl | methyl | H |
| 2-22-1208 | S | N-Ph | b1 | b1 | H |
| 2-22-1209 | S | N-Ph | b2 | b2 | H |
| 2-22-1210 | S | N-Ph | b3 | b3 | H |
| 2-22-1211 | S | N-Ph | H | H | H |
| 2-22-1212 | S | N-Ph | t-bu | t-bu | H |
| 2-22-1213 | S | N-Ph | methyl | methyl | H |
| 2-22-1214 | S | N-Ph | b1 | b1 | H |
| 2-22-1215 | S | N-Ph | b2 | b2 | H |
| 2-22-1216 | S | N-Ph | b3 | b3 | H |
| 2-22-1217 | S | N-Ph | H | H | a1 |
| 2-22-1218 | S | N-Ph | t-bu | t-bu | a1 |
| 2-22-1219 | S | N-Ph | methyl | methyl | a1 |
| 2-22-1220 | S | N-Ph | b1 | b1 | a1 |
| 2-22-1221 | S | N-Ph | b2 | b2 | a1 |
| 2-22-1222 | S | N-Ph | b3 | b3 | a1 |
| 2-22-1223 | S | N-Ph | H | H | a2 |
| 2-22-1224 | S | N-Ph | t-bu | t-bu | a2 |
| 2-22-1225 | S | N-Ph | methyl | methyl | a2 |
| 2-22-1226 | S | N-Ph | b1 | b1 | a2 |
| 2-22-1227 | S | N-Ph | b2 | b2 | a2 |
| 2-22-1228 | S | N-Ph | b3 | b3 | a2 |
| 2-22-1229 | S | N-Ph | H | H | a3 |
| 2-22-1230 | S | N-Ph | t-bu | t-bu | a3 |
| 2-22-1231 | S | N-Ph | methyl | methyl | a3 |
| 2-22-1232 | S | N-Ph | b1 | b1 | a3 |
| 2-22-1233 | S | N-Ph | b2 | b2 | a3 |
| 2-22-1234 | S | N-Ph | b3 | b3 | a3 |
| 2-22-1235 | S | N-Ph | H | H | b1 |
| 2-22-1236 | S | N-Ph | t-bu | t-bu | b1 |
| 2-22-1237 | S | N-Ph | methyl | methyl | b1 |
| 2-22-1238 | S | N-Ph | b1 | b1 | b1 |
| 2-22-1239 | S | N-Ph | b2 | b2 | b1 |
| 2-22-1240 | S | N-Ph | b3 | b3 | b1 |
| 2-22-1241 | S | N-Ph | H | H | b2 |
| 2-22-1242 | S | N-Ph | t-bu | t-bu | b2 |
| 2-22-1243 | S | N-Ph | methyl | methyl | b2 |
| 2-22-1244 | S | N-Ph | b1 | b1 | b2 |
| 2-22-1245 | S | N-Ph | b2 | b2 | b2 |
| 2-22-1246 | S | N-Ph | b3 | b3 | b2 |
| 2-22-1247 | S | N-Ph | H | H | b3 |
| 2-22-1248 | S | N-Ph | t-bu | t-bu | b3 |
| 2-22-1249 | S | N-Ph | methyl | methyl | b3 |
| 2-22-1250 | S | N-Ph | b1 | b1 | b3 |
| 2-22-1251 | S | N-Ph | b2 | b2 | b3 |
| 2-22-1252 | S | N-Ph | b3 | b3 | b3 |
| 2-22-1253 | S | N-Ph | H | H | b5 |
| 2-22-1254 | S | N-Ph | t-bu | t-bu | b5 |
| 2-22-1255 | S | N-Ph | methyl | methyl | b5 |
| 2-22-1256 | S | N-Ph | b1 | b1 | b5 |
| 2-22-1257 | S | N-Ph | b2 | b2 | b5 |
| 2-22-1258 | S | N-Ph | b3 | b3 | b5 |
| 2-22-1259 | S | N-Ph | H | H | b6 |
| 2-22-1260 | S | N-Ph | t-bu | t-bu | b6 |
| 2-22-1261 | S | N-Ph | methyl | methyl | b6 |
| 2-22-1262 | S | N-Ph | b1 | b1 | b6 |
| 2-22-1263 | S | N-Ph | b2 | b2 | b6 |
| 2-22-1264 | S | N-Ph | b3 | b3 | b6 |
| 2-22-1265 | S | N-Ph | H | H | b7 |
| 2-22-1266 | S | N-Ph | t-bu | t-bu | b7 |
| 2-22-1267 | S | N-Ph | methyl | methyl | b7 |
| 2-22-1268 | S | N-Ph | b1 | b1 | b7 |
| 2-22-1269 | S | N-Ph | b2 | b2 | b7 |
| 2-22-1270 | S | N-Ph | b3 | b3 | b7 |

Group 2-23
The compounds are represented by the formula 2-23 below, wherein CY₁ is a benzene group, X₁ is B, and Y1, Y2, Y3, R2b, R3a, (R1)a1 are each shown in the table 23 below, 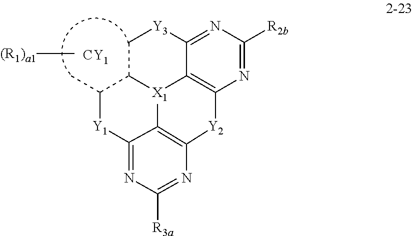
TABLE 23

| Compound | | | | | |
|---|---|---|---|---|---|
| No. | Y1/Y3 | Y2 | R2b | R3a | (R1)a1 |
| 2-23-001 | N—Ph | N—Ph | H | H | H |
| 2-23-002 | N—Ph | N—Ph | t-bu | t-bu | H |
| 2-23-003 | N—Ph | N—Ph | methyl | methyl | H |
| 2-23-004 | N—Ph | N—Ph | t-bu | t-bu | t-bu |
| 2-23-005 | N—Ph | N—Ph | methyl | methyl | methyl |
| 2-23-006 | N—Ph | N—Ph | b1 | b1 | H |
| 2-23-007 | N—Ph | N—Ph | b2 | b2 | H |
| 2-23-008 | N—Ph | N—Ph | b3 | b3 | H |
| 2-23-009 | N—Ph | N—Ph | H | H | a1 |
| 2-23-010 | N—Ph | N—Ph | t-bu | t-bu | a1 |
| 2-23-011 | N—Ph | N—Ph | methyl | methyl | a1 |
| 2-23-012 | N—Ph | N—Ph | b1 | b1 | a1 |
| 2-23-013 | N—Ph | N—Ph | b2 | b2 | a1 |
| 2-23-014 | N—Ph | N—Ph | b3 | b3 | a1 |
| 2-23-015 | N—Ph | N—Ph | H | H | a2 |
| 2-23-016 | N—Ph | N—Ph | t-bu | t-bu | a2 |
| 2-23-017 | N—Ph | N—Ph | methyl | methyl | a2 |
| 2-23-018 | N—Ph | N—Ph | b1 | b1 | a2 |
| 2-23-019 | N—Ph | N—Ph | b2 | b2 | a2 |
| 2-23-020 | N—Ph | N—Ph | b3 | b3 | a2 |
| 2-23-021 | N—Ph | N—Ph | H | H | a3 |
| 2-23-022 | N—Ph | N—Ph | t-bu | t-bu | a3 |
| 2-23-023 | N—Ph | N—Ph | methyl | methyl | a3 |
| 2-23-024 | N—Ph | N—Ph | b1 | b1 | a3 |
| 2-23-025 | N—Ph | N—Ph | b2 | b2 | a3 |
| 2-23-026 | N—Ph | N—Ph | b3 | b3 | a3 |
| 2-23-027 | N—Ph | N—Ph | H | H | a4 |
| 2-23-028 | N—Ph | N—Ph | t-bu | t-bu | a4 |
| 2-23-029 | N—Ph | N—Ph | methyl | methyl | a4 |
| 2-23-030 | N—Ph | N—Ph | b1 | b1 | a4 |
| 2-23-031 | N—Ph | N—Ph | b2 | b2 | a4 |
| 2-23-032 | N—Ph | N—Ph | b3 | b3 | a4 |
| 2-23-033 | N—Ph | N—Ph | H | H | a5 |
| 2-23-034 | N—Ph | N—Ph | t-bu | t-bu | a5 |
| 2-23-035 | N—Ph | N—Ph | methyl | methyl | a5 |
| 2-23-036 | N—Ph | N—Ph | b1 | b1 | a5 |
| 2-23-037 | N—Ph | N—Ph | b2 | b2 | a5 |
| 2-23-038 | N—Ph | N—Ph | b3 | b3 | a5 |
| 2-23-039 | N—Ph | N—Ph | H | H | a6 |
| 2-23-040 | N—Ph | N—Ph | t-bu | t-bu | a6 |
| 2-23-041 | N—Ph | N—Ph | methyl | methyl | a6 |
| 2-23-042 | N—Ph | N—Ph | b1 | b1 | a6 |
| 2-23-043 | N—Ph | N—Ph | b2 | b2 | a6 |
| 2-23-044 | N—Ph | N—Ph | b3 | b3 | a6 |
| 2-23-045 | N—Ph | N—Ph | H | H | b1 |
| 2-23-046 | N—Ph | N—Ph | t-bu | t-bu | b1 |
| 2-23-047 | N—Ph | N—Ph | methyl | methyl | b1 |
| 2-23-048 | N—Ph | N—Ph | b1 | b1 | b1 |
| 2-23-049 | N—Ph | N—Ph | b2 | b2 | b1 |
| 2-23-050 | N—Ph | N—Ph | b3 | b3 | b1 |
| 2-23-051 | N—Ph | N—Ph | H | H | b2 |
| 2-23-052 | N—Ph | N—Ph | t-bu | t-bu | b2 |
| 2-23-053 | N—Ph | N—Ph | methyl | methyl | b2 |
| 2-23-054 | N—Ph | N—Ph | b1 | b1 | b2 |
| 2-23-055 | N—Ph | N—Ph | b2 | b2 | b2 |
| 2-23-056 | N—Ph | N—Ph | b3 | b3 | b2 |
| 2-23-057 | N—Ph | N—Ph | H | H | b3 |
| 2-23-058 | N—Ph | N—Ph | t-bu | t-bu | b3 |
| 2-23-059 | N—Ph | N—Ph | methyl | methyl | b3 |
| 2-23-060 | N—Ph | N—Ph | b1 | b1 | b3 |
| 2-23-061 | N—Ph | N—Ph | b2 | b2 | b3 |
| 2-23-062 | N—Ph | N—Ph | b3 | b3 | b3 |
| 2-23-063 | N—Ph | N—Ph | H | H | b4 |
| 2-23-064 | N—Ph | N—Ph | t-bu | t-bu | b4 |
| 2-23-065 | N—Ph | N—Ph | methyl | methyl | b4 |
| 2-23-066 | N—Ph | N—Ph | b1 | b1 | b4 |
| 2-23-067 | N—Ph | N—Ph | b2 | b2 | b4 |
| 2-23-068 | N—Ph | N—Ph | b3 | b3 | b4 |
| 2-23-069 | N—Ph | N—Ph | H | H | b5 |
| 2-23-070 | N—Ph | N—Ph | t-bu | t-bu | b5 |
| 2-23-071 | N—Ph | N—Ph | methyl | methyl | b5 |
| 2-23-072 | N—Ph | N—Ph | b1 | b1 | b5 |
| 2-23-073 | N—Ph | N—Ph | b2 | b2 | b5 |
| 2-23-074 | N—Ph | N—Ph | b3 | b3 | b5 |
| 2-23-075 | N—Ph | N—Ph | H | H | b6 |
| 2-23-076 | N—Ph | N—Ph | t-bu | t-bu | b6 |
| 2-23-077 | N—Ph | N—Ph | methyl | methyl | b6 |
| 2-23-078 | N—Ph | N—Ph | b1 | b1 | b6 |
| 2-23-079 | N—Ph | N—Ph | b2 | b2 | b6 |
| 2-23-080 | N—Ph | N—Ph | b3 | b3 | b6 |
| 2-23-081 | N—Ph | N—Ph | H | H | b7 |
| 2-23-082 | N—Ph | N—Ph | t-bu | t-bu | b7 |
| 2-23-083 | N—Ph | N—Ph | methyl | methyl | b7 |
| 2-23-084 | N—Ph | N—Ph | b1 | b1 | b7 |
| 2-23-085 | N—Ph | N—Ph | b2 | b2 | b7 |
| 2-23-086 | N—Ph | N—Ph | b3 | b3 | b7 |
| 2-23-087 | N—Ph | N—Ph | H | H | b8 |
| 2-23-088 | N—Ph | N—Ph | t-bu | t-bu | b8 |
| 2-23-089 | N—Ph | N—Ph | methyl | methyl | b8 |
| 2-23-090 | N—Ph | N—Ph | b1 | b1 | b8 |
| 2-23-091 | N—Ph | N—Ph | b2 | b2 | b8 |
| 2-23-092 | N—Ph | N—Ph | b3 | b3 | b8 |
| 2-23-093 | N—Ph | N—Ph | H | H | b9 |
| 2-23-094 | N—Ph | N—Ph | t-bu | t-bu | b9 |
| 2-23-095 | N—Ph | N—Ph | methyl | methyl | b9 |
| 2-23-096 | N—Ph | N—Ph | b1 | b1 | b9 |
| 2-23-097 | N—Ph | N—Ph | b2 | b2 | b9 |
| 2-23-098 | N—Ph | N—Ph | b3 | b3 | b9 |
| 2-23-099 | N—Ph | N—Ph | H | H | b10 |
| 2-23-100 | N—Ph | N—Ph | t-bu | t-bu | b10 |
| 2-23-101 | N—Ph | N—Ph | methyl | methyl | b10 |
| 2-23-102 | N—Ph | N—Ph | b1 | b1 | b10 |
| 2-23-103 | N—Ph | N—Ph | b2 | b2 | b10 |
| 2-23-104 | N—Ph | N—Ph | b3 | b3 | b10 |
| 2-23-105 | N—Ph | N—Ph | H | H | H |
| 2-23-106 | N—Ph | N—Ph | t-bu | t-bu | H |
| 2-23-107 | N—Ph | N—Ph | methyl | methyl | H |
| 2-23-108 | N—Ph | N—Ph | b1 | b1 | H |
| 2-23-109 | N—Ph | N—Ph | b2 | b2 | H |
| 2-23-110 | N—Ph | N—Ph | b3 | b3 | H |
| 2-23-111 | N—Ph | N—Ph | H | H | H |
| 2-23-112 | N—Ph | N—Ph | t-bu | t-bu | H |
| 2-23-113 | N—Ph | N—Ph | methyl | methyl | H |
| 2-23-114 | N—Ph | N—Ph | b1 | b1 | H |
| 2-23-115 | N—Ph | N—Ph | b2 | b2 | H |
| 2-23-116 | N—Ph | N—Ph | b3 | b3 | H |
| 2-23-117 | N—Ph | N—Ph | H | H | H |
| 2-23-118 | N—Ph | N—Ph | t-bu | t-bu | H |
| 2-23-119 | N—Ph | N—Ph | methyl | methyl | H |
| 2-23-120 | N—Ph | N—Ph | b1 | b1 | H |
| 2-23-121 | N—Ph | N—Ph | b2 | b2 | H |
| 2-23-122 | N—Ph | N—Ph | b3 | b3 | H |
| 2-23-123 | N—Ph | N—Ph | H | H | H |
| 2-23-124 | N—Ph | N—Ph | t-bu | t-bu | H |
| 2-23-125 | N—Ph | N—Ph | methyl | methyl | H |
| 2-23-126 | N—Ph | N—Ph | b1 | b1 | H |
| 2-23-127 | N—Ph | N—Ph | b2 | b2 | H |
| 2-23-128 | N—Ph | N—Ph | b3 | b3 | H |
| 2-23-129 | N—Ph | N—Ph | H | H | H |
| 2-23-130 | N—Ph | N—Ph | t-bu | t-bu | H |
| 2-23-131 | N—Ph | N—Ph | methyl | methyl | H |
| 2-23-132 | N—Ph | N—Ph | b1 | b1 | H |
| 2-23-133 | N—Ph | N—Ph | b2 | b2 | H |
| 2-23-134 | N—Ph | N—Ph | b3 | b3 | H |
| 2-23-135 | N—Ph | N—Ph | H | H | H |
| 2-23-136 | N—Ph | N—Ph | t-bu | t-bu | H |
| 2-23-137 | N—Ph | N—Ph | methyl | methyl | H |
| 2-23-138 | N—Ph | N—Ph | b1 | b1 | H |
| 2-23-139 | N—Ph | N—Ph | b2 | b2 | H |
| 2-23-140 | N—Ph | N—Ph | b3 | b3 | H |
| 2-23-141 | N—Ph | N—Ph | H | H | H |
| 2-23-142 | N—Ph | N—Ph | t-bu | t-bu | H |
| 2-23-143 | N—Ph | N—Ph | methyl | methyl | H |
| 2-23-144 | N—Ph | N—Ph | b1 | b1 | H |
| 2-23-145 | N—Ph | N—Ph | b2 | b2 | H |
| 2-23-146 | N—Ph | N—Ph | b3 | b3 | H |
| 2-23-147 | N—Ph | N—Ph | H | H | H |
| 2-23-148 | N—Ph | N—Ph | t-bu | t-bu | H |
| 2-23-149 | N—Ph | N—Ph | methyl | methyl | H |
| 2-23-150 | N—Ph | N—Ph | b1 | b1 | H |
| 2-23-151 | N—Ph | N—Ph | b2 | b2 | H |
| 2-23-152 | N—Ph | N—Ph | b3 | b3 | H |
| 2-23-153 | N—Ph | N—Ph | H | H | H |
| 2-23-154 | N—Ph | N—Ph | t-bu | t-bu | H |
| 2-23-155 | N—Ph | N—Ph | methyl | methyl | H |
| 2-23-156 | N—Ph | N—Ph | b1 | b1 | H |
| 2-23-157 | N—Ph | N—Ph | b2 | b2 | H |
| 2-23-158 | N—Ph | N—Ph | b3 | b3 | H |
| 2-23-159 | N—Ph | N—Ph | H | H | H |
| 2-23-160 | N—Ph | N—Ph | t-bu | t-bu | H |
| 2-23-161 | N—Ph | N—Ph | methyl | methyl | H |
| 2-23-162 | N—Ph | N—Ph | b1 | b1 | H |
| 2-23-163 | N—Ph | N—Ph | b2 | b2 | H |
| 2-23-164 | N—Ph | N—Ph | b3 | b3 | H |
| 2-23-165 | N—Ph | N—Ph | H | H | H |
| 2-23-166 | N—Ph | N—Ph | t-bu | t-bu | H |
| 2-23-167 | N—Ph | N—Ph | methyl | methyl | H |
| 2-23-168 | N—Ph | N—Ph | b1 | b1 | H |
| 2-23-169 | N—Ph | N—Ph | b2 | b2 | H |
| 2-23-170 | N—Ph | N—Ph | b3 | b3 | H |
| 2-23-171 | N—Ph | N—Ph | H | H | H |
| 2-23-172 | N—Ph | N—Ph | t-bu | t-bu | H |
| 2-23-173 | N—Ph | N—Ph | methyl | methyl | H |
| 2-23-174 | N—Ph | N—Ph | b1 | b1 | H |
| 2-23-175 | N—Ph | N—Ph | b2 | b2 | H |
| 2-23-176 | N—Ph | N—Ph | b3 | b3 | H |
| 2-23-177 | N—Ph | N—Ph | H | H | H |
| 2-23-178 | N—Ph | N—Ph | t-bu | t-bu | H |
| 2-23-179 | N—Ph | N—Ph | methyl | methyl | H |
| 2-23-180 | N—Ph | N—Ph | b1 | b1 | H |
| 2-23-181 | N—Ph | N—Ph | b2 | b2 | H |
| 2-23-182 | N—Ph | N—Ph | b3 | b3 | H |
| 2-23-183 | N—Ph | N—Ph | H | H | H |
| 2-23-184 | N—Ph | N—Ph | t-bu | t-bu | H |
| 2-23-185 | N—Ph | N—Ph | methyl | methyl | H |
| 2-23-186 | N—Ph | N—Ph | b1 | b1 | H |
| 2-23-187 | N—Ph | N—Ph | b2 | b2 | H |
| 2-23-188 | N—Ph | N—Ph | b3 | b3 | H |
| 2-23-189 | N—Ph | N—Ph | H | H | H |
| 2-23-190 | N—Ph | N—Ph | t-bu | t-bu | H |
| 2-23-191 | N—Ph | N—Ph | methyl | methyl | H |
| 2-23-192 | N—Ph | N—Ph | b1 | b1 | H |
| 2-23-193 | N—Ph | N—Ph | b2 | b2 | H |
| 2-23-194 | N—Ph | N—Ph | b3 | b3 | H |
| 2-23-195 | N—Ph | N—Ph | H | H | H |
| 2-23-196 | N—Ph | N—Ph | t-bu | t-bu | H |
| 2-23-197 | N—Ph | N—Ph | methyl | methyl | H |
| 2-23-198 | N—Ph | N—Ph | b1 | b1 | H |
| 2-23-199 | N—Ph | N—Ph | b2 | b2 | H |
| 2-23-200 | N—Ph | N—Ph | b3 | b3 | H |
| 2-23-201 | N—Ph | N—Ph | H | H | a1 |
| 2-23-202 | N—Ph | N—Ph | t-bu | t-bu | a1 |
| 2-23-203 | N—Ph | N—Ph | methyl | methyl | a1 |
| 2-23-204 | N—Ph | N—Ph | b1 | b1 | a1 |
| 2-23-205 | N—Ph | N—Ph | b2 | b2 | a1 |
| 2-23-206 | N—Ph | N—Ph | b3 | b3 | a1 |
| 2-23-207 | N—Ph | N—Ph | H | H | a2 |
| 2-23-208 | N—Ph | N—Ph | t-bu | t-bu | a2 |
| 2-23-209 | N—Ph | N—Ph | methyl | methyl | a2 |
| 2-23-210 | N—Ph | N—Ph | b1 | b1 | a2 |
| 2-23-211 | N—Ph | N—Ph | b2 | b2 | a2 |
| 2-23-212 | N—Ph | N—Ph | b3 | b3 | a2 |
| 2-23-213 | N—Ph | N—Ph | H | H | a3 |
| 2-23-214 | N—Ph | N—Ph | t-bu | t-bu | a3 |
| 2-23-215 | N—Ph | N—Ph | methyl | methyl | a3 |
| 2-23-216 | N—Ph | N—Ph | b1 | b1 | a3 |
| 2-23-217 | N—Ph | N—Ph | b2 | b2 | a3 |
| 2-23-218 | N—Ph | N—Ph | b3 | b3 | a3 |
| 2-23-219 | N—Ph | N—Ph | H | H | b1 |
| 2-23-220 | N—Ph | N—Ph | t-bu | t-bu | b1 |
| 2-23-221 | N—Ph | N—Ph | methyl | methyl | b1 |
| 2-23-222 | N—Ph | N—Ph | b1 | b1 | b1 |
| 2-23-223 | N—Ph | N—Ph | b2 | b2 | b1 |
| 2-23-224 | N—Ph | N—Ph | b3 | b3 | b1 |
| 2-23-225 | N—Ph | N—Ph | H | H | b2 |
| 2-23-226 | N—Ph | N—Ph | t-bu | t-bu | b2 |
| 2-23-227 | N—Ph | N—Ph | methyl | methyl | b2 |
| 2-23-228 | N—Ph | N—Ph | b1 | b1 | b2 |
| 2-23-229 | N—Ph | N—Ph | b2 | b2 | b2 |
| 2-23-230 | N—Ph | N—Ph | b3 | b3 | b2 |
| 2-23-231 | N—Ph | N—Ph | H | H | b3 |
| 2-23-232 | N—Ph | N—Ph | t-bu | t-bu | b3 |
| 2-23-233 | N—Ph | N—Ph | methyl | methyl | b3 |
| 2-23-234 | N—Ph | N—Ph | b1 | b1 | b3 |
| 2-23-235 | N—Ph | N—Ph | b2 | b2 | b3 |
| 2-23-236 | N—Ph | N—Ph | b3 | b3 | b3 |
| 2-23-237 | N—Ph | N—Ph | H | H | b5 |
| 2-23-238 | N—Ph | N—Ph | t-bu | t-bu | b5 |
| 2-23-239 | N—Ph | N—Ph | methyl | methyl | b5 |
| 2-23-240 | N—Ph | N—Ph | b1 | b1 | b5 |
| 2-23-241 | N—Ph | N—Ph | b2 | b2 | b5 |
| 2-23-242 | N—Ph | N—Ph | b3 | b3 | b5 |
| 2-23-243 | N—Ph | N—Ph | H | H | b6 |
| 2-23-244 | N—Ph | N—Ph | t-bu | t-bu | b6 |
| 2-23-245 | N—Ph | N—Ph | methyl | methyl | b6 |
| 2-23-246 | N—Ph | N—Ph | b1 | b1 | b6 |
| 2-23-247 | N—Ph | N—Ph | b2 | b2 | b6 |
| 2-23-248 | N—Ph | N—Ph | b3 | b3 | b6 |
| 2-23-249 | N—Ph | N—Ph | H | H | b7 |
| 2-23-250 | N—Ph | N—Ph | t-bu | t-bu | b7 |
| 2-23-251 | N—Ph | N—Ph | methyl | methyl | b7 |
| 2-23-252 | N—Ph | N—Ph | b1 | b1 | b7 |
| 2-23-253 | N—Ph | N—Ph | b2 | b2 | b7 |
| 2-23-254 | N—Ph | N—Ph | b3 | b3 | b7 |
| 2-23-255 | N-tolyl | N-tolyl | H | H | H |
| 2-23-256 | N-tolyl | N-tolyl | t-bu | t-bu | H |
| 2-23-257 | N-tolyl | N-tolyl | methyl | methyl | H |
| 2-23-258 | N-tolyl | N-tolyl | t-bu | t-bu | t-bu |
| 2-23-259 | N-tolyl | N-tolyl | methyl | methyl | methyl |
| 2-23-260 | N-tolyl | N-tolyl | b1 | b1 | H |
| 2-23-261 | N-tolyl | N-tolyl | b2 | b2 | H |
| 2-23-262 | N-tolyl | N-tolyl | b3 | b3 | H |
| 2-23-263 | N-tolyl | N-tolyl | H | H | a1 |
| 2-23-264 | N-tolyl | N-tolyl | t-bu | t-bu | a1 |
| 2-23-265 | N-tolyl | N-tolyl | methyl | methyl | a1 |
| 2-23-266 | N-tolyl | N-tolyl | b1 | b1 | a1 |
| 2-23-267 | N-tolyl | N-tolyl | b2 | b2 | a1 |
| 2-23-268 | N-tolyl | N-tolyl | b3 | b3 | a1 |
| 2-23-269 | N-tolyl | N-tolyl | H | H | a2 |
| 2-23-270 | N-tolyl | N-tolyl | t-bu | t-bu | a2 |
| 2-23-271 | N-tolyl | N-tolyl | methyl | methyl | a2 |
| 2-23-272 | N-tolyl | N-tolyl | b1 | b1 | a2 |
| 2-23-273 | N-tolyl | N-tolyl | b2 | b2 | a2 |
| 2-23-274 | N-tolyl | N-tolyl | b3 | b3 | a2 |
| 2-23-275 | N-tolyl | N-tolyl | H | H | a3 |
| 2-23-276 | N-tolyl | N-tolyl | t-bu | t-bu | a3 |
| 2-23-277 | N-tolyl | N-tolyl | methyl | methyl | a3 |
| 2-23-278 | N-tolyl | N-tolyl | b1 | b1 | a3 |
| 2-23-279 | N-tolyl | N-tolyl | b2 | b2 | a3 |
| 2-23-280 | N-tolyl | N-tolyl | b3 | b3 | a3 |
| 2-23-281 | N-tolyl | N-tolyl | H | H | a4 |
| 2-23-282 | N-tolyl | N-tolyl | t-bu | t-bu | a4 |
| 2-23-283 | N-tolyl | N-tolyl | methyl | methyl | a4 |
| 2-23-284 | N-tolyl | N-tolyl | b1 | b1 | a4 |
| 2-23-285 | N-tolyl | N-tolyl | b2 | b2 | a4 |
| 2-23-286 | N-tolyl | N-tolyl | b3 | b3 | a4 |
| 2-23-287 | N-tolyl | N-tolyl | H | H | a5 |
| 2-23-288 | N-tolyl | N-tolyl | t-bu | t-bu | a5 |
| 2-23-289 | N-tolyl | N-tolyl | methyl | methyl | a5 |
| 2-23-290 | N-tolyl | N-tolyl | b1 | b1 | a5 |
| 2-23-291 | N-tolyl | N-tolyl | b2 | b2 | a5 |
| 2-23-292 | N-tolyl | N-tolyl | b3 | b3 | a5 |
| 2-23-293 | N-tolyl | N-tolyl | H | H | a6 |
| 2-23-294 | N-tolyl | N-tolyl | t-bu | t-bu | a6 |
| 2-23-295 | N-tolyl | N-tolyl | methyl | methyl | a6 |
| 2-23-296 | N-tolyl | N-tolyl | b1 | b1 | a6 |
| 2-23-297 | N-tolyl | N-tolyl | b2 | b2 | a6 |
| 2-23-298 | N-tolyl | N-tolyl | b3 | b3 | a6 |
| 2-23-299 | N-tolyl | N-tolyl | H | H | b1 |
| 2-23-300 | N-tolyl | N-tolyl | t-bu | t-bu | b1 |
| 2-23-301 | N-tolyl | N-tolyl | methyl | methyl | b1 |
| 2-23-302 | N-tolyl | N-tolyl | b1 | b1 | b1 |
| 2-23-303 | N-tolyl | N-tolyl | b2 | b2 | b1 |
| 2-23-304 | N-tolyl | N-tolyl | b3 | b3 | b1 |
| 2-23-305 | N-tolyl | N-tolyl | H | H | b2 |
| 2-23-306 | N-tolyl | N-tolyl | t-bu | t-bu | b2 |
| 2-23-307 | N-tolyl | N-tolyl | methyl | methyl | b2 |
| 2-23-308 | N-tolyl | N-tolyl | b1 | b1 | b2 |
| 2-23-309 | N-tolyl | N-tolyl | b2 | b2 | b2 |
| 2-23-310 | N-tolyl | N-tolyl | b3 | b3 | b2 |
| 2-23-311 | N-tolyl | N-tolyl | H | H | b3 |
| 2-23-312 | N-tolyl | N-tolyl | t-bu | t-bu | b3 |
| 2-23-313 | N-tolyl | N-tolyl | methyl | methyl | b3 |
| 2-23-314 | N-tolyl | N-tolyl | b1 | b1 | b3 |
| 2-23-315 | N-tolyl | N-tolyl | b2 | b2 | b3 |
| 2-23-316 | N-tolyl | N-tolyl | b3 | b3 | b3 |
| 2-23-317 | N-tolyl | N-tolyl | H | H | b4 |
| 2-23-318 | N-tolyl | N-tolyl | t-bu | t-bu | b4 |
| 2-23-319 | N-tolyl | N-tolyl | methyl | methyl | b4 |
| 2-23-320 | N-tolyl | N-tolyl | b1 | b1 | b4 |
| 2-23-321 | N-tolyl | N-tolyl | b2 | b2 | b4 |
| 2-23-322 | N-tolyl | N-tolyl | b3 | b3 | b4 |
| 2-23-323 | N-tolyl | N-tolyl | H | H | b5 |
| 2-23-324 | N-tolyl | N-tolyl | t-bu | t-bu | b5 |
| 2-23-325 | N-tolyl | N-tolyl | methyl | methyl | b5 |
| 2-23-326 | N-tolyl | N-tolyl | b1 | b1 | b5 |
| 2-23-327 | N-tolyl | N-tolyl | b2 | b2 | b5 |
| 2-23-328 | N-tolyl | N-tolyl | b3 | b3 | b5 |
| 2-23-329 | N-tolyl | N-tolyl | H | H | b6 |
| 2-23-330 | N-tolyl | N-tolyl | t-bu | t-bu | b6 |
| 2-23-331 | N-tolyl | N-tolyl | methyl | methyl | b6 |
| 2-23-332 | N-tolyl | N-tolyl | b1 | b1 | b6 |
| 2-23-333 | N-tolyl | N-tolyl | b2 | b2 | b6 |
| 2-23-334 | N-tolyl | N-tolyl | b3 | b3 | b6 |
| 2-23-335 | N-tolyl | N-tolyl | H | H | b7 |
| 2-23-336 | N-tolyl | N-tolyl | t-bu | t-bu | b7 |
| 2-23-337 | N-tolyl | N-tolyl | methyl | methyl | b7 |
| 2-23-338 | N-tolyl | N-tolyl | b1 | b1 | b7 |
| 2-23-339 | N-tolyl | N-tolyl | b2 | b2 | b7 |
| 2-23-340 | N-tolyl | N-tolyl | b3 | b3 | b7 |
| 2-23-341 | N-tolyl | N-tolyl | H | H | b8 |
| 2-23-342 | N-tolyl | N-tolyl | t-bu | t-bu | b8 |
| 2-23-343 | N-tolyl | N-tolyl | methyl | methyl | b8 |
| 2-23-344 | N-tolyl | N-tolyl | b1 | b1 | b8 |
| 2-23-345 | N-tolyl | N-tolyl | b2 | b2 | b8 |
| 2-23-346 | N-tolyl | N-tolyl | b3 | b3 | b8 |
| 2-23-347 | N-tolyl | N-tolyl | H | H | b9 |
| 2-23-348 | N-tolyl | N-tolyl | t-bu | t-bu | b9 |
| 2-23-349 | N-tolyl | N-tolyl | methyl | methyl | b9 |
| 2-23-350 | N-tolyl | N-tolyl | b1 | b1 | b9 |
| 2-23-351 | N-tolyl | N-tolyl | b2 | b2 | b9 |
| 2-23-352 | N-tolyl | N-tolyl | b3 | b3 | b9 |
| 2-23-353 | N-tolyl | N-tolyl | H | H | b10 |
| 2-23-354 | N-tolyl | N-tolyl | t-bu | t-bu | b10 |
| 2-23-355 | N-tolyl | N-tolyl | methyl | methyl | b10 |
| 2-23-356 | N-tolyl | N-tolyl | b1 | b1 | b10 |
| 2-23-357 | N-tolyl | N-tolyl | b2 | b2 | b10 |
| 2-23-358 | N-tolyl | N-tolyl | b3 | b3 | b10 |
| 2-23-359 | N-tolyl | N-tolyl | H | H | H |
| 2-23-360 | N-tolyl | N-tolyl | t-bu | t-bu | H |
| 2-23-361 | N-tolyl | N-tolyl | methyl | methyl | H |
| 2-23-362 | N-tolyl | N-tolyl | b1 | b1 | H |
| 2-23-363 | N-tolyl | N-tolyl | b2 | b2 | H |
| 2-23-364 | N-tolyl | N-tolyl | b3 | b3 | H |
| 2-23-365 | N-tolyl | N-tolyl | H | H | H |
| 2-23-366 | N-tolyl | N-tolyl | t-bu | t-bu | H |
| 2-23-367 | N-tolyl | N-tolyl | methyl | methyl | H |
| 2-23-368 | N-tolyl | N-tolyl | b1 | b1 | H |
| 2-23-369 | N-tolyl | N-tolyl | b2 | b2 | H |
| 2-23-370 | N-tolyl | N-tolyl | b3 | b3 | H |
| 2-23-371 | N-tolyl | N-tolyl | H | H | H |
| 2-23-372 | N-tolyl | N-tolyl | t-bu | t-bu | H |
| 2-23-373 | N-tolyl | N-tolyl | methyl | methyl | H |
| 2-23-374 | N-tolyl | N-tolyl | b1 | b1 | H |
| 2-23-375 | N-tolyl | N-tolyl | b2 | b2 | H |
| 2-23-376 | N-tolyl | N-tolyl | b3 | b3 | H |
| 2-23-377 | N-tolyl | N-tolyl | H | H | H |
| 2-23-378 | N-tolyl | N-tolyl | t-bu | t-bu | H |
| 2-23-379 | N-tolyl | N-tolyl | methyl | methyl | H |
| 2-23-380 | N-tolyl | N-tolyl | b1 | b1 | H |
| 2-23-381 | N-tolyl | N-tolyl | b2 | b2 | H |
| 2-23-382 | N-tolyl | N-tolyl | b3 | b3 | H |
| 2-23-383 | N-tolyl | N-tolyl | H | H | H |
| 2-23-384 | N-tolyl | N-tolyl | t-bu | t-bu | H |
| 2-23-385 | N-tolyl | N-tolyl | methyl | methyl | H |
| 2-23-386 | N-tolyl | N-tolyl | b1 | b1 | H |
| 2-23-387 | N-tolyl | N-tolyl | b2 | b2 | H |
| 2-23-388 | N-tolyl | N-tolyl | b3 | b3 | H |
| 2-23-389 | N-tolyl | N-tolyl | H | H | H |
| 2-23-390 | N-tolyl | N-tolyl | t-bu | t-bu | H |
| 2-23-391 | N-tolyl | N-tolyl | methyl | methyl | H |
| 2-23-392 | N-tolyl | N-tolyl | b1 | b1 | H |
| 2-23-393 | N-tolyl | N-tolyl | b2 | b2 | H |
| 2-23-394 | N-tolyl | N-tolyl | b3 | b3 | H |
| 2-23-395 | N-tolyl | N-tolyl | H | H | H |
| 2-23-396 | N-tolyl | N-tolyl | t-bu | t-bu | H |
| 2-23-397 | N-tolyl | N-tolyl | methyl | methyl | H |
| 2-23-398 | N-tolyl | N-tolyl | b1 | b1 | H |
| 2-23-399 | N-tolyl | N-tolyl | b2 | b2 | H |
| 2-23-400 | N-tolyl | N-tolyl | b3 | b3 | H |
| 2-23-401 | N-tolyl | N-tolyl | H | H | H |
| 2-23-402 | N-tolyl | N-tolyl | t-bu | t-bu | H |
| 2-23-403 | N-tolyl | N-tolyl | methyl | methyl | H |
| 2-23-404 | N-tolyl | N-tolyl | b1 | b1 | H |
| 2-23-405 | N-tolyl | N-tolyl | b2 | b2 | H |
| 2-23-406 | N-tolyl | N-tolyl | b3 | b3 | H |
| 2-23-407 | N-tolyl | N-tolyl | H | H | H |
| 2-23-408 | N-tolyl | N-tolyl | t-bu | t-bu | H |
| 2-23-409 | N-tolyl | N-tolyl | methyl | methyl | H |
| 2-23-410 | N-tolyl | N-tolyl | b1 | b1 | H |
| 2-23-411 | N-tolyl | N-tolyl | b2 | b2 | H |
| 2-23-412 | N-tolyl | N-tolyl | b3 | b3 | H |
| 2-23-413 | N-tolyl | N-tolyl | H | H | H |
| 2-23-414 | N-tolyl | N-tolyl | t-bu | t-bu | H |
| 2-23-415 | N-tolyl | N-tolyl | methyl | methyl | H |
| 2-23-416 | N-tolyl | N-tolyl | b1 | b1 | H |
| 2-23-417 | N-tolyl | N-tolyl | b2 | b2 | H |
| 2-23-418 | N-tolyl | N-tolyl | b3 | b3 | H |
| 2-23-419 | N-tolyl | N-tolyl | H | H | H |
| 2-23-420 | N-tolyl | N-tolyl | t-bu | t-bu | H |
| 2-23-421 | N-tolyl | N-tolyl | methyl | methyl | H |
| 2-23-422 | N-tolyl | N-tolyl | b1 | b1 | H |
| 2-23-423 | N-tolyl | N-tolyl | b2 | b2 | H |
| 2-23-424 | N-tolyl | N-tolyl | b3 | b3 | H |
| 2-23-425 | N-tolyl | N-tolyl | H | H | H |
| 2-23-426 | N-tolyl | N-tolyl | t-bu | t-bu | H |
| 2-23-427 | N-tolyl | N-tolyl | methyl | methyl | H |
| 2-23-428 | N-tolyl | N-tolyl | b1 | b1 | H |
| 2-23-429 | N-tolyl | N-tolyl | b2 | b2 | H |
| 2-23-430 | N-tolyl | N-tolyl | b3 | b3 | H |
| 2-23-431 | N-tolyl | N-tolyl | H | H | H |
| 2-23-432 | N-tolyl | N-tolyl | t-bu | t-bu | H |
| 2-23-433 | N-tolyl | N-tolyl | methyl | methyl | H |
| 2-23-434 | N-tolyl | N-tolyl | b1 | b1 | H |
| 2-23-435 | N-tolyl | N-tolyl | b2 | b2 | H |
| 2-23-436 | N-tolyl | N-tolyl | b3 | b3 | H |
| 2-23-437 | N-tolyl | N-tolyl | H | H | H |
| 2-23-438 | N-tolyl | N-tolyl | t-bu | t-bu | H |
| 2-23-439 | N-tolyl | N-tolyl | methyl | methyl | H |
| 2-23-440 | N-tolyl | N-tolyl | b1 | b1 | H |
| 2-23-441 | N-tolyl | N-tolyl | b2 | b2 | H |
| 2-23-442 | N-tolyl | N-tolyl | b3 | b3 | H |
| 2-23-443 | N-tolyl | N-tolyl | H | H | H |
| 2-23-444 | N-tolyl | N-tolyl | t-bu | t-bu | H |
| 2-23-445 | N-tolyl | N-tolyl | methyl | methyl | H |
| 2-23-446 | N-tolyl | N-tolyl | b1 | b1 | H |
| 2-23-447 | N-tolyl | N-tolyl | b2 | b2 | H |
| 2-23-448 | N-tolyl | N-tolyl | b3 | b3 | H |
| 2-23-449 | N-tolyl | N-tolyl | H | H | H |
| 2-23-450 | N-tolyl | N-tolyl | t-bu | t-bu | H |
| 2-23-451 | N-tolyl | N-tolyl | methyl | methyl | H |
| 2-23-452 | N-tolyl | N-tolyl | b1 | b1 | H |
| 2-23-453 | N-tolyl | N-tolyl | b2 | b2 | H |
| 2-23-454 | N-tolyl | N-tolyl | b3 | b3 | H |
| 2-23-455 | N-tolyl | N-tolyl | H | H | a1 |
| 2-23-456 | N-tolyl | N-tolyl | t-bu | t-bu | a1 |
| 2-23-457 | N-tolyl | N-tolyl | methyl | methyl | a1 |
| 2-23-458 | N-tolyl | N-tolyl | b1 | b1 | a1 |
| 2-23-459 | N-tolyl | N-tolyl | b2 | b2 | a1 |
| 2-23-460 | N-tolyl | N-tolyl | b3 | b3 | a1 |
| 2-23-461 | N-tolyl | N-tolyl | H | H | a2 |
| 2-23-462 | N-tolyl | N-tolyl | t-bu | t-bu | a2 |
| 2-23-463 | N-tolyl | N-tolyl | methyl | methyl | a2 |
| 2-23-464 | N-tolyl | N-tolyl | b1 | b1 | a2 |
| 2-23-465 | N-tolyl | N-tolyl | b2 | b2 | a2 |
| 2-23-466 | N-tolyl | N-tolyl | b3 | b3 | a2 |
| 2-23-467 | N-tolyl | N-tolyl | H | H | a3 |
| 2-23-468 | N-tolyl | N-tolyl | t-bu | t-bu | a3 |
| 2-23-469 | N-tolyl | N-tolyl | methyl | methyl | a3 |
| 2-23-470 | N-tolyl | N-tolyl | b1 | b1 | a3 |
| 2-23-471 | N-tolyl | N-tolyl | b2 | b2 | a3 |
| 2-23-472 | N-tolyl | N-tolyl | b3 | b3 | a3 |
| 2-23-473 | N-tolyl | N-tolyl | H | H | b1 |
| 2-23-474 | N-tolyl | N-tolyl | t-bu | t-bu | b1 |
| 2-23-475 | N-tolyl | N-tolyl | methyl | methyl | b1 |
| 2-23-476 | N-tolyl | N-tolyl | b1 | b1 | b1 |
| 2-23-477 | N-tolyl | N-tolyl | b2 | b2 | b1 |
| 2-23-478 | N-tolyl | N-tolyl | b3 | b3 | b1 |
| 2-23-479 | N-tolyl | N-tolyl | H | H | b2 |
| 2-23-480 | N-tolyl | N-tolyl | t-bu | t-bu | b2 |
| 2-23-481 | N-tolyl | N-tolyl | methyl | methyl | b2 |
| 2-23-482 | N-tolyl | N-tolyl | b1 | b1 | b2 |
| 2-23-483 | N-tolyl | N-tolyl | b2 | b2 | b2 |
| 2-23-484 | N-tolyl | N-tolyl | b3 | b3 | b2 |
| 2-23-485 | N-tolyl | N-tolyl | H | H | b3 |
| 2-23-486 | N-tolyl | N-tolyl | t-bu | t-bu | b3 |
| 2-23-487 | N-tolyl | N-tolyl | methyl | methyl | b3 |
| 2-23-488 | N-tolyl | N-tolyl | b1 | b1 | b3 |
| 2-23-489 | N-tolyl | N-tolyl | b2 | b2 | b3 |
| 2-23-490 | N-tolyl | N-tolyl | b3 | b3 | b3 |
| 2-23-491 | N-tolyl | N-tolyl | H | H | b5 |
| 2-23-492 | N-tolyl | N-tolyl | t-bu | t-bu | b5 |
| 2-23-493 | N-tolyl | N-tolyl | methyl | methyl | b5 |
| 2-23-494 | N-tolyl | N-tolyl | b1 | b1 | b5 |
| 2-23-495 | N-tolyl | N-tolyl | b2 | b2 | b5 |
| 2-23-496 | N-tolyl | N-tolyl | b3 | b3 | b5 |
| 2-23-497 | N-tolyl | N-tolyl | H | H | b6 |
| 2-23-498 | N-tolyl | N-tolyl | t-bu | t-bu | b6 |
| 2-23-499 | N-tolyl | N-tolyl | methyl | methyl | b6 |
| 2-23-500 | N-tolyl | N-tolyl | b1 | b1 | b6 |
| 2-23-501 | N-tolyl | N-tolyl | b2 | b2 | b6 |
| 2-23-502 | N-tolyl | N-tolyl | b3 | b3 | b6 |
| 2-23-503 | N-tolyl | N-tolyl | H | H | b7 |
| 2-23-504 | N-tolyl | N-tolyl | t-bu | t-bu | b7 |
| 2-23-505 | N-tolyl | N-tolyl | methyl | methyl | b7 |
| 2-23-506 | N-tolyl | N-tolyl | b1 | b1 | b7 |
| 2-23-507 | N-tolyl | N-tolyl | b2 | b2 | b7 |
| 2-23-508 | N-tolyl | N-tolyl | b3 | b3 | b7 |
| 2-23-509 | N-Biph | N-Biph | H | H | H |
| 2-23-510 | N-Biph | N-Biph | t-bu | t-bu | H |
| 2-23-511 | N-Biph | N-Biph | methyl | methyl | H |
| 2-23-512 | N-Biph | N-Biph | t-bu | t-bu | t-bu |
| 2-23-513 | N-Biph | N-Biph | methyl | methyl | methyl |
| 2-23-514 | N-Biph | N-Biph | b1 | b1 | H |
| 2-23-515 | N-Biph | N-Biph | b2 | b2 | H |
| 2-23-516 | N-Biph | N-Biph | b3 | b3 | H |
| 2-23-517 | N-Biph | N-Biph | H | H | a1 |
| 2-23-518 | N-Biph | N-Biph | t-bu | t-bu | a1 |
| 2-23-519 | N-Biph | N-Biph | methyl | methyl | a1 |
| 2-23-520 | N-Biph | N-Biph | b1 | b1 | a1 |
| 2-23-521 | N-Biph | N-Biph | b2 | b2 | a1 |
| 2-23-522 | N-Biph | N-Biph | b3 | b3 | a1 |
| 2-23-523 | N-Biph | N-Biph | H | H | a2 |
| 2-23-524 | N-Biph | N-Biph | t-bu | t-bu | a2 |
| 2-23-525 | N-Biph | N-Biph | methyl | methyl | a2 |
| 2-23-526 | N-Biph | N-Biph | b1 | b1 | a2 |
| 2-23-527 | N-Biph | N-Biph | b2 | b2 | a2 |
| 2-23-528 | N-Biph | N-Biph | b3 | b3 | a2 |
| 2-23-529 | N-Biph | N-Biph | H | H | a3 |
| 2-23-530 | N-Biph | N-Biph | t-bu | t-bu | a3 |
| 2-23-531 | N-Biph | N-Biph | methyl | methyl | a3 |
| 2-23-532 | N-Biph | N-Biph | b1 | b1 | a3 |
| 2-23-533 | N-Biph | N-Biph | b2 | b2 | a3 |
| 2-23-534 | N-Biph | N-Biph | b3 | b3 | a3 |
| 2-23-535 | N-Biph | N-Biph | H | H | a4 |
| 2-23-536 | N-Biph | N-Biph | t-bu | t-bu | a4 |
| 2-23-537 | N-Biph | N-Biph | methyl | methyl | a4 |
| 2-23-538 | N-Biph | N-Biph | b1 | b1 | a4 |
| 2-23-539 | N-Biph | N-Biph | b2 | b2 | a4 |
| 2-23-540 | N-Biph | N-Biph | b3 | b3 | a4 |
| 2-23-541 | N-Biph | N-Biph | H | H | a5 |
| 2-23-542 | N-Biph | N-Biph | t-bu | t-bu | a5 |
| 2-23-543 | N-Biph | N-Biph | methyl | methyl | a5 |
| 2-23-544 | N-Biph | N-Biph | b1 | b1 | a5 |
| 2-23-545 | N-Biph | N-Biph | b2 | b2 | a5 |
| 2-23-546 | N-Biph | N-Biph | b3 | b3 | a5 |
| 2-23-547 | N-Biph | N-Biph | H | H | a6 |
| 2-23-548 | N-Biph | N-Biph | t-bu | t-bu | a6 |
| 2-23-549 | N-Biph | N-Biph | methyl | methyl | a6 |
| 2-23-550 | N-Biph | N-Biph | b1 | b1 | a6 |
| 2-23-551 | N-Biph | N-Biph | b2 | b2 | a6 |
| 2-23-552 | N-Biph | N-Biph | b3 | b3 | a6 |
| 2-23-553 | N-Biph | N-Biph | H | H | b1 |
| 2-23-554 | N-Biph | N-Biph | t-bu | t-bu | b1 |
| 2-23-555 | N-Biph | N-Biph | methyl | methyl | b1 |
| 2-23-556 | N-Biph | N-Biph | b1 | b1 | b1 |
| 2-23-557 | N-Biph | N-Biph | b2 | b2 | b1 |
| 2-23-558 | N-Biph | N-Biph | b3 | b3 | b1 |
| 2-23-559 | N-Biph | N-Biph | H | H | b2 |
| 2-23-560 | N-Biph | N-Biph | t-bu | t-bu | b2 |
| 2-23-561 | N-Biph | N-Biph | methyl | methyl | b2 |
| 2-23-562 | N-Biph | N-Biph | b1 | b1 | b2 |
| 2-23-563 | N-Biph | N-Biph | b2 | b2 | b2 |
| 2-23-564 | N-Biph | N-Biph | b3 | b3 | b2 |
| 2-23-565 | N-Biph | N-Biph | H | H | b3 |
| 2-23-566 | N-Biph | N-Biph | t-bu | t-bu | b3 |
| 2-23-567 | N-Biph | N-Biph | methyl | methyl | b3 |
| 2-23-568 | N-Biph | N-Biph | b1 | b1 | b3 |
| 2-23-569 | N-Biph | N-Biph | b2 | b2 | b3 |
| 2-23-570 | N-Biph | N-Biph | b3 | b3 | b3 |
| 2-23-571 | N-Biph | N-Biph | H | H | b4 |
| 2-23-572 | N-Biph | N-Biph | t-bu | t-bu | b4 |
| 2-23-573 | N-Biph | N-Biph | methyl | methyl | b4 |
| 2-23-574 | N-Biph | N-Biph | b1 | b1 | b4 |
| 2-23-575 | N-Biph | N-Biph | b2 | b2 | b4 |
| 2-23-576 | N-Biph | N-Biph | b3 | b3 | b4 |
| 2-23-577 | N-Biph | N-Biph | H | H | b5 |
| 2-23-578 | N-Biph | N-Biph | t-bu | t-bu | b5 |
| 2-23-579 | N-Biph | N-Biph | methyl | methyl | b5 |
| 2-23-580 | N-Biph | N-Biph | b1 | b1 | b5 |
| 2-23-581 | N-Biph | N-Biph | b2 | b2 | b5 |
| 2-23-582 | N-Biph | N-Biph | b3 | b3 | b5 |
| 2-23-583 | N-Biph | N-Biph | H | H | b6 |
| 2-23-584 | N-Biph | N-Biph | t-bu | t-bu | b6 |
| 2-23-585 | N-Biph | N-Biph | methyl | methyl | b6 |
| 2-23-586 | N-Biph | N-Biph | b1 | b1 | b6 |
| 2-23-587 | N-Biph | N-Biph | b2 | b2 | b6 |
| 2-23-588 | N-Biph | N-Biph | b3 | b3 | b6 |
| 2-23-589 | N-Biph | N-Biph | H | H | b7 |
| 2-23-590 | N-Biph | N-Biph | t-bu | t-bu | b7 |
| 2-23-591 | N-Biph | N-Biph | methyl | methyl | b7 |
| 2-23-592 | N-Biph | N-Biph | b1 | b1 | b7 |
| 2-23-593 | N-Biph | N-Biph | b2 | b2 | b7 |
| 2-23-594 | N-Biph | N-Biph | b3 | b3 | b7 |
| 2-23-595 | N-Biph | N-Biph | H | H | b8 |
| 2-23-596 | N-Biph | N-Biph | t-bu | t-bu | b8 |
| 2-23-597 | N-Biph | N-Biph | methyl | methyl | b8 |
| 2-23-598 | N-Biph | N-Biph | b1 | b1 | b8 |
| 2-23-599 | N-Biph | N-Biph | b2 | b2 | b8 |
| 2-23-600 | N-Biph | N-Biph | b3 | b3 | b8 |
| 2-23-601 | N-Biph | N-Biph | H | H | b9 |
| 2-23-602 | N-Biph | N-Biph | t-bu | t-bu | b9 |
| 2-23-603 | N-Biph | N-Biph | methyl | methyl | b9 |
| 2-23-604 | N-Biph | N-Biph | b1 | b1 | b9 |
| 2-23-605 | N-Biph | N-Biph | b2 | b2 | b9 |
| 2-23-606 | N-Biph | N-Biph | b3 | b3 | b9 |
| 2-23-607 | N-Biph | N-Biph | H | H | b10 |
| 2-23-608 | N-Biph | N-Biph | t-bu | t-bu | b10 |
| 2-23-609 | N-Biph | N-Biph | methyl | methyl | b10 |
| 2-23-610 | N-Biph | N-Biph | b1 | b1 | b10 |
| 2-23-611 | N-Biph | N-Biph | b2 | b2 | b10 |
| 2-23-612 | N-Biph | N-Biph | b3 | b3 | b10 |
| 2-23-613 | N-Biph | N-Biph | H | H | H |
| 2-23-614 | N-Biph | N-Biph | t-bu | t-bu | H |
| 2-23-615 | N-Biph | N-Biph | methyl | methyl | H |
| 2-23-616 | N-Biph | N-Biph | b1 | b1 | H |
| 2-23-617 | N-Biph | N-Biph | b2 | b2 | H |
| 2-23-618 | N-Biph | N-Biph | b3 | b3 | H |
| 2-23-619 | N-Biph | N-Biph | H | H | H |
| 2-23-620 | N-Biph | N-Biph | t-bu | t-bu | H |
| 2-23-621 | N-Biph | N-Biph | methyl | methyl | H |
| 2-23-622 | N-Biph | N-Biph | b1 | b1 | H |
| 2-23-623 | N-Biph | N-Biph | b2 | b2 | H |
| 2-23-624 | N-Biph | N-Biph | b3 | b3 | H |
| 2-23-625 | N-Biph | N-Biph | H | H | H |
| 2-23-626 | N-Biph | N-Biph | t-bu | t-bu | H |
| 2-23-627 | N-Biph | N-Biph | methyl | methyl | H |
| 2-23-628 | N-Biph | N-Biph | b1 | b1 | H |
| 2-23-629 | N-Biph | N-Biph | b2 | b2 | H |
| 2-23-630 | N-Biph | N-Biph | b3 | b3 | H |
| 2-23-631 | N-Biph | N-Biph | H | H | H |
| 2-23-632 | N-Biph | N-Biph | t-bu | t-bu | H |
| 2-23-633 | N-Biph | N-Biph | methyl | methyl | H |
| 2-23-634 | N-Biph | N-Biph | b1 | b1 | H |
| 2-23-635 | N-Biph | N-Biph | b2 | b2 | H |
| 2-23-636 | N-Biph | N-Biph | b3 | b3 | H |
| 2-23-637 | N-Biph | N-Biph | H | H | H |
| 2-23-638 | N-Biph | N-Biph | t-bu | t-bu | H |
| 2-23-639 | N-Biph | N-Biph | methyl | methyl | H |
| 2-23-640 | N-Biph | N-Biph | b1 | b1 | H |
| 2-23-641 | N-Biph | N-Biph | b2 | b2 | H |
| 2-23-642 | N-Biph | N-Biph | b3 | b3 | H |
| 2-23-643 | N-Biph | N-Biph | H | H | H |
| 2-23-644 | N-Biph | N-Biph | t-bu | t-bu | H |
| 2-23-645 | N-Biph | N-Biph | methyl | methyl | H |
| 2-23-646 | N-Biph | N-Biph | b1 | b1 | H |
| 2-23-647 | N-Biph | N-Biph | b2 | b2 | H |
| 2-23-648 | N-Biph | N-Biph | b3 | b3 | H |
| 2-23-649 | N-Biph | N-Biph | H | H | H |
| 2-23-650 | N-Biph | N-Biph | t-bu | t-bu | H |
| 2-23-651 | N-Biph | N-Biph | methyl | methyl | H |
| 2-23-652 | N-Biph | N-Biph | b1 | b1 | H |
| 2-23-653 | N-Biph | N-Biph | b2 | b2 | H |
| 2-23-654 | N-Biph | N-Biph | b3 | b3 | H |
| 2-23-655 | N-Biph | N-Biph | H | H | H |
| 2-23-656 | N-Biph | N-Biph | t-bu | t-bu | H |
| 2-23-657 | N-Biph | N-Biph | methyl | methyl | H |
| 2-23-658 | N-Biph | N-Biph | b1 | b1 | H |
| 2-23-659 | N-Biph | N-Biph | b2 | b2 | H |
| 2-23-660 | N-Biph | N-Biph | b3 | b3 | H |
| 2-23-661 | N-Biph | N-Biph | H | H | H |
| 2-23-662 | N-Biph | N-Biph | t-bu | t-bu | H |
| 2-23-663 | N-Biph | N-Biph | methyl | methyl | H |
| 2-23-664 | N-Biph | N-Biph | b1 | b1 | H |
| 2-23-665 | N-Biph | N-Biph | b2 | b2 | H |
| 2-23-666 | N-Biph | N-Biph | b3 | b3 | H |
| 2-23-667 | N-Biph | N-Biph | H | H | H |
| 2-23-668 | N-Biph | N-Biph | t-bu | t-bu | H |
| 2-23-669 | N-Biph | N-Biph | methyl | methyl | H |
| 2-23-670 | N-Biph | N-Biph | b1 | b1 | H |
| 2-23-671 | N-Biph | N-Biph | b2 | b2 | H |
| 2-23-672 | N-Biph | N-Biph | b3 | b3 | H |
| 2-23-673 | N-Biph | N-Biph | H | H | H |
| 2-23-674 | N-Biph | N-Biph | t-bu | t-bu | H |
| 2-23-675 | N-Biph | N-Biph | methyl | methyl | H |
| 2-23-676 | N-Biph | N-Biph | b1 | b1 | H |
| 2-23-677 | N-Biph | N-Biph | b2 | b2 | H |
| 2-23-678 | N-Biph | N-Biph | b3 | b3 | H |
| 2-23-679 | N-Biph | N-Biph | H | H | H |
| 2-23-680 | N-Biph | N-Biph | t-bu | t-bu | H |
| 2-23-681 | N-Biph | N-Biph | methyl | methyl | H |
| 2-23-682 | N-Biph | N-Biph | b1 | b1 | H |
| 2-23-683 | N-Biph | N-Biph | b2 | b2 | H |
| 2-23-684 | N-Biph | N-Biph | b3 | b3 | H |
| 2-23-685 | N-Biph | N-Biph | H | H | H |
| 2-23-686 | N-Biph | N-Biph | t-bu | t-bu | H |
| 2-23-687 | N-Biph | N-Biph | methyl | methyl | H |
| 2-23-688 | N-Biph | N-Biph | b1 | b1 | H |
| 2-23-689 | N-Biph | N-Biph | b2 | b2 | H |
| 2-23-690 | N-Biph | N-Biph | b3 | b3 | H |
| 2-23-691 | N-Biph | N-Biph | H | H | H |
| 2-23-692 | N-Biph | N-Biph | t-bu | t-bu | H |
| 2-23-693 | N-Biph | N-Biph | methyl | methyl | H |
| 2-23-694 | N-Biph | N-Biph | b1 | b1 | H |
| 2-23-695 | N-Biph | N-Biph | b2 | b2 | H |
| 2-23-696 | N-Biph | N-Biph | b3 | b3 | H |
| 2-23-697 | N-Biph | N-Biph | H | H | H |
| 2-23-698 | N-Biph | N-Biph | t-bu | t-bu | H |
| 2-23-699 | N-Biph | N-Biph | methyl | methyl | H |
| 2-23-700 | N-Biph | N-Biph | b1 | b1 | H |
| 2-23-701 | N-Biph | N-Biph | b2 | b2 | H |
| 2-23-702 | N-Biph | N-Biph | b3 | b3 | H |
| 2-23-703 | N-Biph | N-Biph | H | H | H |
| 2-23-704 | N-Biph | N-Biph | t-bu | t-bu | H |
| 2-23-705 | N-Biph | N-Biph | methyl | methyl | H |
| 2-23-706 | N-Biph | N-Biph | b1 | b1 | H |
| 2-23-707 | N-Biph | N-Biph | b2 | b2 | H |
| 2-23-708 | N-Biph | N-Biph | b3 | b3 | H |
| 2-23-709 | N-Biph | N-Biph | H | H | a1 |
| 2-23-710 | N-Biph | N-Biph | t-bu | t-bu | a1 |
| 2-23-711 | N-Biph | N-Biph | methyl | methyl | a1 |
| 2-23-712 | N-Biph | N-Biph | b1 | b1 | a1 |
| 2-23-713 | N-Biph | N-Biph | b2 | b2 | a1 |
| 2-23-714 | N-Biph | N-Biph | b3 | b3 | a1 |
| 2-23-715 | N-Biph | N-Biph | H | H | a2 |
| 2-23-716 | N-Biph | N-Biph | t-bu | t-bu | a2 |
| 2-23-717 | N-Biph | N-Biph | methyl | methyl | a2 |
| 2-23-718 | N-Biph | N-Biph | b1 | b1 | a2 |
| 2-23-719 | N-Biph | N-Biph | b2 | b2 | a2 |
| 2-23-720 | N-Biph | N-Biph | b3 | b3 | a2 |
| 2-23-721 | N-Biph | N-Biph | H | H | a3 |
| 2-23-722 | N-Biph | N-Biph | t-bu | t-bu | a3 |
| 2-23-723 | N-Biph | N-Biph | methyl | methyl | a3 |
| 2-23-724 | N-Biph | N-Biph | b1 | b1 | a3 |
| 2-23-725 | N-Biph | N-Biph | b2 | b2 | a3 |
| 2-23-726 | N-Biph | N-Biph | b3 | b3 | a3 |
| 2-23-727 | N-Biph | N-Biph | H | H | b1 |
| 2-23-728 | N-Biph | N-Biph | t-bu | t-bu | b1 |
| 2-23-729 | N-Biph | N-Biph | methyl | methyl | b1 |
| 2-23-730 | N-Biph | N-Biph | b1 | b1 | b1 |
| 2-23-731 | N-Biph | N-Biph | b2 | b2 | b1 |
| 2-23-732 | N-Biph | N-Biph | b3 | b3 | b1 |
| 2-23-733 | N-Biph | N-Biph | H | H | b2 |
| 2-23-734 | N-Biph | N-Biph | t-bu | t-bu | b2 |
| 2-23-735 | N-Biph | N-Biph | methyl | methyl | b2 |
| 2-23-736 | N-Biph | N-Biph | b1 | b1 | b2 |
| 2-23-737 | N-Biph | N-Biph | b2 | b2 | b2 |
| 2-23-738 | N-Biph | N-Biph | b3 | b3 | b2 |
| 2-23-739 | N-Biph | N-Biph | H | H | b3 |
| 2-23-740 | N-Biph | N-Biph | t-bu | t-bu | b3 |
| 2-23-741 | N-Biph | N-Biph | methyl | methyl | b3 |
| 2-23-742 | N-Biph | N-Biph | b1 | b1 | b3 |
| 2-23-743 | N-Biph | N-Biph | b2 | b2 | b3 |
| 2-23-744 | N-Biph | N-Biph | b3 | b3 | b3 |
| 2-23-745 | N-Biph | N-Biph | H | H | b5 |
| 2-23-746 | N-Biph | N-Biph | t-bu | t-bu | b5 |
| 2-23-747 | N-Biph | N-Biph | methyl | methyl | b5 |
| 2-23-748 | N-Biph | N-Biph | b1 | b1 | b5 |
| 2-23-749 | N-Biph | N-Biph | b2 | b2 | b5 |
| 2-23-750 | N-Biph | N-Biph | b3 | b3 | b5 |
| 2-23-751 | N-Biph | N-Biph | H | H | b6 |
| 2-23-752 | N-Biph | N-Biph | t-bu | t-bu | b6 |
| 2-23-753 | N-Biph | N-Biph | methyl | methyl | b6 |
| 2-23-754 | N-Biph | N-Biph | b1 | b1 | b6 |
| 2-23-755 | N-Biph | N-Biph | b2 | b2 | b6 |
| 2-23-756 | N-Biph | N-Biph | b3 | b3 | b6 |
| 2-23-757 | N-Biph | N-Biph | H | H | b7 |
| 2-23-758 | N-Biph | N-Biph | t-bu | t-bu | b7 |
| 2-23-759 | N-Biph | N-Biph | methyl | methyl | b7 |
| 2-23-760 | N-Biph | N-Biph | b1 | b1 | b7 |
| 2-23-761 | N-Biph | N-Biph | b2 | b2 | b7 |
| 2-23-762 | N-Biph | N-Biph | b3 | b3 | b7 |
| 2-23-763 | N—Ph | O | H | H | H |
| 2-23-764 | N—Ph | O | t-bu | t-bu | H |
| 2-23-765 | N—Ph | O | methyl | methyl | H |
| 2-23-766 | N—Ph | O | t-bu | t-bu | t-bu |
| 2-23-767 | N—Ph | O | methyl | methyl | methyl |
| 2-23-768 | N—Ph | O | b1 | b1 | H |
| 2-23-769 | N—Ph | O | b2 | b2 | H |
| 2-23-770 | N—Ph | O | b3 | b3 | H |
| 2-23-771 | N—Ph | O | H | H | a1 |
| 2-23-772 | N—Ph | O | t-bu | t-bu | a1 |
| 2-23-773 | N—Ph | O | methyl | methyl | a1 |
| 2-23-774 | N—Ph | O | b1 | b1 | a1 |
| 2-23-775 | N—Ph | O | b2 | b2 | a1 |
| 2-23-776 | N—Ph | O | b3 | b3 | a1 |
| 2-23-777 | N—Ph | O | H | H | a2 |
| 2-23-778 | N—Ph | O | t-bu | t-bu | a2 |
| 2-23-779 | N—Ph | O | methyl | methyl | a2 |
| 2-23-780 | N—Ph | O | b1 | b1 | a2 |
| 2-23-781 | N—Ph | O | b2 | b2 | a2 |
| 2-23-782 | N—Ph | O | b3 | b3 | a2 |
| 2-23-783 | N—Ph | O | H | H | a3 |
| 2-23-784 | N—Ph | O | t-bu | t-bu | a3 |
| 2-23-785 | N—Ph | O | methyl | methyl | a3 |
| 2-23-786 | N—Ph | O | b1 | b1 | a3 |
| 2-23-787 | N—Ph | O | b2 | b2 | a3 |
| 2-23-788 | N—Ph | O | b3 | b3 | a3 |
| 2-23-789 | N—Ph | O | H | H | a4 |
| 2-23-790 | N—Ph | O | t-bu | t-bu | a4 |
| 2-23-791 | N—Ph | O | methyl | methyl | a4 |
| 2-23-792 | N—Ph | O | b1 | b1 | a4 |
| 2-23-793 | N—Ph | O | b2 | b2 | a4 |
| 2-23-794 | N—Ph | O | b3 | b3 | a4 |
| 2-23-795 | N—Ph | O | H | H | a5 |
| 2-23-796 | N—Ph | O | t-bu | t-bu | a5 |
| 2-23-797 | N—Ph | O | methyl | methyl | a5 |
| 2-23-798 | N—Ph | O | b1 | b1 | a5 |
| 2-23-799 | N—Ph | O | b2 | b2 | a5 |
| 2-23-800 | N—Ph | O | b3 | b3 | a5 |
| 2-23-801 | N—Ph | O | H | H | a6 |
| 2-23-802 | N—Ph | O | t-bu | t-bu | a6 |
| 2-23-803 | N—Ph | O | methyl | methyl | a6 |
| 2-23-804 | N—Ph | O | b1 | b1 | a6 |
| 2-23-805 | N—Ph | O | b2 | b2 | a6 |
| 2-23-806 | N—Ph | O | b3 | b3 | a6 |
| 2-23-807 | N—Ph | O | H | H | b1 |
| 2-23-808 | N—Ph | O | t-bu | t-bu | b1 |
| 2-23-809 | N—Ph | O | methyl | methyl | b1 |
| 2-23-810 | N—Ph | O | b1 | b1 | b1 |
| 2-23-811 | N—Ph | O | b2 | b2 | b1 |
| 2-23-812 | N—Ph | O | b3 | b3 | b1 |
| 2-23-813 | N—Ph | O | H | H | b2 |
| 2-23-814 | N—Ph | O | t-bu | t-bu | b2 |
| 2-23-815 | N—Ph | O | methyl | methyl | b2 |
| 2-23-816 | N—Ph | O | b1 | b1 | b2 |
| 2-23-817 | N—Ph | O | b2 | b2 | b2 |
| 2-23-818 | N—Ph | O | b3 | b3 | b2 |
| 2-23-819 | N—Ph | O | H | H | b3 |
| 2-23-820 | N—Ph | O | t-bu | t-bu | b3 |
| 2-23-821 | N—Ph | O | methyl | methyl | b3 |
| 2-23-822 | N—Ph | O | b1 | b1 | b3 |
| 2-23-823 | N—Ph | O | b2 | b2 | b3 |
| 2-23-824 | N—Ph | O | b3 | b3 | b3 |
| 2-23-825 | N—Ph | O | H | H | b4 |
| 2-23-826 | N—Ph | O | t-bu | t-bu | b4 |
| 2-23-827 | N—Ph | O | methyl | methyl | b4 |
| 2-23-828 | N—Ph | O | b1 | b1 | b4 |
| 2-23-829 | N—Ph | O | b2 | b2 | b4 |
| 2-23-830 | N—Ph | O | b3 | b3 | b4 |
| 2-23-831 | N—Ph | O | H | H | b5 |
| 2-23-832 | N—Ph | O | t-bu | t-bu | b5 |
| 2-23-833 | N—Ph | O | methyl | methyl | b5 |
| 2-23-834 | N—Ph | O | b1 | b1 | b5 |
| 2-23-835 | N—Ph | O | b2 | b2 | b5 |
| 2-23-836 | N—Ph | O | b3 | b3 | b5 |
| 2-23-837 | N—Ph | O | H | H | b6 |
| 2-23-838 | N—Ph | O | t-bu | t-bu | b6 |
| 2-23-839 | N—Ph | O | methyl | methyl | b6 |
| 2-23-840 | N—Ph | O | b1 | b1 | b6 |
| 2-23-841 | N—Ph | O | b2 | b2 | b6 |
| 2-23-842 | N—Ph | O | b3 | b3 | b6 |
| 2-23-843 | N—Ph | O | H | H | b7 |
| 2-23-844 | N—Ph | O | t-bu | t-bu | b7 |
| 2-23-845 | N—Ph | O | methyl | methyl | b7 |
| 2-23-846 | N—Ph | O | b1 | b1 | b7 |
| 2-23-847 | N—Ph | O | b2 | b2 | b7 |
| 2-23-848 | N—Ph | O | b3 | b3 | b7 |
| 2-23-849 | N—Ph | O | H | H | b8 |
| 2-23-850 | N—Ph | O | t-bu | t-bu | b8 |
| 2-23-851 | N—Ph | O | methyl | methyl | b8 |
| 2-23-852 | N—Ph | O | b1 | b1 | b8 |
| 2-23-853 | N—Ph | O | b2 | b2 | b8 |
| 2-23-854 | N—Ph | O | b3 | b3 | b8 |
| 2-23-855 | N—Ph | O | H | H | b9 |
| 2-23-856 | N—Ph | O | t-bu | t-bu | b9 |
| 2-23-857 | N—Ph | O | methyl | methyl | b9 |
| 2-23-858 | N—Ph | O | b1 | b1 | b9 |
| 2-23-859 | N—Ph | O | b2 | b2 | b9 |
| 2-23-860 | N—Ph | O | b3 | b3 | b9 |
| 2-23-861 | N—Ph | O | H | H | b10 |
| 2-23-862 | N—Ph | O | t-bu | t-bu | b10 |
| 2-23-863 | N—Ph | O | methyl | methyl | b10 |
| 2-23-864 | N—Ph | O | b1 | b1 | b10 |
| 2-23-865 | N—Ph | O | b2 | b2 | b10 |
| 2-23-866 | N—Ph | O | b3 | b3 | b10 |
| 2-23-867 | N—Ph | O | H | H | H |
| 2-23-868 | N—Ph | O | t-bu | t-bu | H |
| 2-23-869 | N—Ph | O | methyl | methyl | H |
| 2-23-870 | N—Ph | O | b1 | b1 | H |
| 2-23-871 | N—Ph | O | b2 | b2 | H |
| 2-23-872 | N—Ph | O | b3 | b3 | H |
| 2-23-873 | N—Ph | O | H | H | H |
| 2-23-874 | N—Ph | O | t-bu | t-bu | H |
| 2-23-875 | N—Ph | O | methyl | methyl | H |
| 2-23-876 | N—Ph | O | b1 | b1 | H |
| 2-23-877 | N—Ph | O | b2 | b2 | H |
| 2-23-878 | N—Ph | O | b3 | b3 | H |
| 2-23-879 | N—Ph | O | H | H | H |
| 2-23-880 | N—Ph | O | t-bu | t-bu | H |
| 2-23-881 | N—Ph | O | methyl | methyl | H |
| 2-23-882 | N—Ph | O | b1 | b1 | H |
| 2-23-883 | N—Ph | O | b2 | b2 | H |
| 2-23-884 | N—Ph | O | b3 | b3 | H |
| 2-23-885 | N—Ph | O | H | H | H |
| 2-23-886 | N—Ph | O | t-bu | t-bu | H |
| 2-23-887 | N—Ph | O | methyl | methyl | H |
| 2-23-888 | N—Ph | O | b1 | b1 | H |
| 2-23-889 | N—Ph | O | b2 | b2 | H |
| 2-23-890 | N—Ph | O | b3 | b3 | H |
| 2-23-891 | N—Ph | O | H | H | H |
| 2-23-892 | N—Ph | O | t-bu | t-bu | H |
| 2-23-893 | N—Ph | O | methyl | methyl | H |
| 2-23-894 | N—Ph | O | b1 | b1 | H |
| 2-23-895 | N—Ph | O | b2 | b2 | H |
| 2-23-896 | N—Ph | O | b3 | b3 | H |
| 2-23-897 | N—Ph | O | H | H | H |
| 2-23-898 | N—Ph | O | t-bu | t-bu | H |
| 2-23-899 | N—Ph | O | methyl | methyl | H |
| 2-23-900 | N—Ph | O | b1 | b1 | H |
| 2-23-901 | N—Ph | O | b2 | b2 | H |
| 2-23-902 | N—Ph | O | b3 | b3 | H |
| 2-23-903 | N—Ph | O | H | H | H |
| 2-23-904 | N—Ph | O | t-bu | t-bu | H |
| 2-23-905 | N—Ph | O | methyl | methyl | H |
| 2-23-906 | N—Ph | O | b1 | b1 | H |
| 2-23-907 | N—Ph | O | b2 | b2 | H |
| 2-23-908 | N—Ph | O | b3 | b3 | H |
| 2-23-909 | N—Ph | O | H | H | H |
| 2-23-910 | N—Ph | O | t-bu | t-bu | H |
| 2-23-911 | N—Ph | O | methyl | methyl | H |
| 2-23-912 | N—Ph | O | b1 | b1 | H |
| 2-23-913 | N—Ph | O | b2 | b2 | H |
| 2-23-914 | N—Ph | O | b3 | b3 | H |
| 2-23-915 | N—Ph | O | H | H | H |
| 2-23-916 | N—Ph | O | t-bu | t-bu | H |
| 2-23-917 | N—Ph | O | methyl | methyl | H |
| 2-23-918 | N—Ph | O | b1 | b1 | H |
| 2-23-919 | N—Ph | O | b2 | b2 | H |
| 2-23-920 | N—Ph | O | b3 | b3 | H |
| 2-23-921 | N—Ph | O | H | H | H |
| 2-23-922 | N—Ph | O | t-bu | t-bu | H |
| 2-23-923 | N—Ph | O | methyl | methyl | H |
| 2-23-924 | N—Ph | O | b1 | b1 | H |
| 2-23-925 | N—Ph | O | b2 | b2 | H |
| 2-23-926 | N—Ph | O | b3 | b3 | H |
| 2-23-927 | N—Ph | O | H | H | H |
| 2-23-928 | N—Ph | O | t-bu | t-bu | H |
| 2-23-929 | N—Ph | O | methyl | methyl | H |
| 2-23-930 | N—Ph | O | b1 | b1 | H |
| 2-23-931 | N—Ph | O | b2 | b2 | H |
| 2-23-932 | N—Ph | O | b3 | b3 | H |
| 2-23-933 | N—Ph | O | H | H | H |
| 2-23-934 | N—Ph | O | t-bu | t-bu | H |
| 2-23-935 | N—Ph | O | methyl | methyl | H |
| 2-23-936 | N—Ph | O | b1 | b1 | H |
| 2-23-937 | N—Ph | O | b2 | b2 | H |
| 2-23-938 | N—Ph | O | b3 | b3 | H |
| 2-23-939 | N—Ph | O | H | H | H |
| 2-23-940 | N—Ph | O | t-bu | t-bu | H |
| 2-23-941 | N—Ph | O | methyl | methyl | H |
| 2-23-942 | N—Ph | O | b1 | b1 | H |
| 2-23-943 | N—Ph | O | b2 | b2 | H |
| 2-23-944 | N—Ph | O | b3 | b3 | H |
| 2-23-945 | N—Ph | O | H | H | H |
| 2-23-946 | N—Ph | O | t-bu | t-bu | H |
| 2-23-947 | N—Ph | O | methyl | methyl | H |
| 2-23-948 | N—Ph | O | b1 | b1 | H |
| 2-23-949 | N—Ph | O | b2 | b2 | H |
| 2-23-950 | N—Ph | O | b3 | b3 | H |
| 2-23-951 | N—Ph | O | H | H | H |
| 2-23-952 | N—Ph | O | t-bu | t-bu | H |
| 2-23-953 | N—Ph | O | methyl | methyl | H |
| 2-23-954 | N—Ph | O | b1 | b1 | H |
| 2-23-955 | N—Ph | O | b2 | b2 | H |
| 2-23-956 | N—Ph | O | b3 | b3 | H |
| 2-23-957 | N—Ph | O | H | H | H |
| 2-23-958 | N—Ph | O | t-bu | t-bu | H |
| 2-23-959 | N—Ph | O | methyl | methyl | H |
| 2-23-960 | N—Ph | O | b1 | b1 | H |
| 2-23-961 | N—Ph | O | b2 | b2 | H |
| 2-23-962 | N—Ph | O | b3 | b3 | H |
| 2-23-963 | N—Ph | O | H | H | a1 |
| 2-23-964 | N—Ph | O | t-bu | t-bu | a1 |
| 2-23-965 | N—Ph | O | methyl | methyl | a1 |
| 2-23-966 | N—Ph | O | b1 | b1 | a1 |
| 2-23-967 | N—Ph | O | b2 | b2 | a1 |
| 2-23-968 | N—Ph | O | b3 | b3 | a1 |
| 2-23-969 | N—Ph | O | H | H | a2 |
| 2-23-970 | N—Ph | O | t-bu | t-bu | a2 |
| 2-23-971 | N—Ph | O | methyl | methyl | a2 |
| 2-23-972 | N—Ph | O | b1 | b1 | a2 |
| 2-23-973 | N—Ph | O | b2 | b2 | a2 |
| 2-23-974 | N—Ph | O | b3 | b3 | a2 |
| 2-23-975 | N—Ph | O | H | H | a3 |
| 2-23-976 | N—Ph | O | t-bu | t-bu | a3 |
| 2-23-977 | N—Ph | O | methyl | methyl | a3 |
| 2-23-978 | N—Ph | O | b1 | b1 | a3 |
| 2-23-979 | N—Ph | O | b2 | b2 | a3 |
| 2-23-980 | N—Ph | O | b3 | b3 | a3 |
| 2-23-981 | N—Ph | O | H | H | b1 |
| 2-23-982 | N—Ph | O | t-bu | t-bu | b1 |
| 2-23-983 | N—Ph | O | methyl | methyl | b1 |
| 2-23-984 | N—Ph | O | b1 | b1 | b1 |
| 2-23-985 | N—Ph | O | b2 | b2 | b1 |
| 2-23-986 | N—Ph | O | b3 | b3 | b1 |
| 2-23-987 | N—Ph | O | H | H | b2 |
| 2-23-988 | N—Ph | O | t-bu | t-bu | b2 |
| 2-23-989 | N—Ph | O | methyl | methyl | b2 |
| 2-23-990 | N—Ph | O | b1 | b1 | b2 |
| 2-23-991 | N—Ph | O | b2 | b2 | b2 |
| 2-23-992 | N—Ph | O | b3 | b3 | b2 |
| 2-23-993 | N—Ph | O | H | H | b3 |
| 2-23-994 | N—Ph | O | t-bu | t-bu | b3 |
| 2-23-995 | N—Ph | O | methyl | methyl | b3 |
| 2-23-996 | N—Ph | O | b1 | b1 | b3 |
| 2-23-997 | N—Ph | O | b2 | b2 | b3 |
| 2-23-998 | N—Ph | O | b3 | b3 | b3 |
| 2-23-999 | N—Ph | O | H | H | b5 |
| 2-23-1000 | N—Ph | O | t-bu | t-bu | b5 |
| 2-23-1001 | N—Ph | O | methyl | methyl | b5 |
| 2-23-1002 | N—Ph | O | b1 | b1 | b5 |
| 2-23-1003 | N—Ph | O | b2 | b2 | b5 |
| 2-23-1004 | N—Ph | O | b3 | b3 | b5 |
| 2-23-1005 | N—Ph | O | H | H | b6 |
| 2-23-1006 | N—Ph | O | t-bu | t-bu | b6 |
| 2-23-1007 | N—Ph | O | methyl | methyl | b6 |
| 2-23-1008 | N—Ph | O | b1 | b1 | b6 |
| 2-23-1009 | N—Ph | O | b2 | b2 | b6 |
| 2-23-1010 | N—Ph | O | b3 | b3 | b6 |
| 2-23-1011 | N—Ph | O | H | H | b7 |
| 2-23-1012 | N—Ph | O | t-bu | t-bu | b7 |
| 2-23-1013 | N—Ph | O | methyl | methyl | b7 |
| 2-23-1014 | N—Ph | O | b1 | b1 | b7 |
| 2-23-1015 | N—Ph | O | b2 | b2 | b7 |
| 2-23-1016 | N—Ph | O | b3 | b3 | b7 |
| 2-23-1017 | N—Ph | S | H | H | H |
| 2-23-1018 | N—Ph | S | t-bu | t-bu | H |
| 2-23-1019 | N—Ph | S | methyl | methyl | H |
| 2-23-1020 | N—Ph | S | t-bu | t-bu | t-bu |
| 2-23-1021 | N—Ph | S | methyl | methyl | methyl |
| 2-23-1022 | N—Ph | S | b1 | b1 | H |
| 2-23-1023 | N—Ph | S | b2 | b2 | H |
| 2-23-1024 | N—Ph | S | b3 | b3 | H |
| 2-23-1025 | N—Ph | S | H | H | a1 |
| 2-23-1026 | N—Ph | S | t-bu | t-bu | a1 |
| 2-23-1027 | N—Ph | S | methyl | methyl | a1 |
| 2-23-1028 | N—Ph | S | b1 | b1 | a1 |
| 2-23-1029 | N—Ph | S | b2 | b2 | a1 |
| 2-23-1030 | N—Ph | S | b3 | b3 | a1 |
| 2-23-1031 | N—Ph | S | H | H | a2 |
| 2-23-1032 | N—Ph | S | t-bu | t-bu | a2 |
| 2-23-1033 | N—Ph | S | methyl | methyl | a2 |
| 2-23-1034 | N—Ph | S | b1 | b1 | a2 |
| 2-23-1035 | N—Ph | S | b2 | b2 | a2 |
| 2-23-1036 | N—Ph | S | b3 | b3 | a2 |
| 2-23-1037 | N—Ph | S | H | H | a3 |
| 2-23-1038 | N—Ph | S | t-bu | t-bu | a3 |
| 2-23-1039 | N—Ph | S | methyl | methyl | a3 |
| 2-23-1040 | N—Ph | S | b1 | b1 | a3 |
| 2-23-1041 | N—Ph | S | b2 | b2 | a3 |
| 2-23-1042 | N—Ph | S | b3 | b3 | a3 |
| 2-23-1043 | N—Ph | S | H | H | a4 |
| 2-23-1044 | N—Ph | S | t-bu | t-bu | a4 |
| 2-23-1045 | N—Ph | S | methyl | methyl | a4 |
| 2-23-1046 | N—Ph | S | b1 | b1 | a4 |
| 2-23-1047 | N—Ph | S | b2 | b2 | a4 |
| 2-23-1048 | N—Ph | S | b3 | b3 | a4 |
| 2-23-1049 | N—Ph | S | H | H | a5 |
| 2-23-1050 | N—Ph | S | t-bu | t-bu | a5 |
| 2-23-1051 | N—Ph | S | methyl | methyl | a5 |
| 2-23-1052 | N—Ph | S | b1 | b1 | a5 |
| 2-23-1053 | N—Ph | S | b2 | b2 | a5 |
| 2-23-1054 | N—Ph | S | b3 | b3 | a5 |
| 2-23-1055 | N—Ph | S | H | H | a6 |
| 2-23-1056 | N—Ph | S | t-bu | t-bu | a6 |
| 2-23-1057 | N—Ph | S | methyl | methyl | a6 |
| 2-23-1058 | N—Ph | S | b1 | b1 | a6 |
| 2-23-1059 | N—Ph | S | b2 | b2 | a6 |
| 2-23-1060 | N—Ph | S | b3 | b3 | a6 |
| 2-23-1061 | N—Ph | S | H | H | b1 |
| 2-23-1062 | N—Ph | S | t-bu | t-bu | b1 |
| 2-23-1063 | N—Ph | S | methyl | methyl | b1 |
| 2-23-1064 | N—Ph | S | b1 | b1 | b1 |
| 2-23-1065 | N—Ph | S | b2 | b2 | b1 |
| 2-23-1066 | N—Ph | S | b3 | b3 | b1 |
| 2-23-1067 | N—Ph | S | H | H | b2 |
| 2-23-1068 | N—Ph | S | t-bu | t-bu | b2 |
| 2-23-1069 | N—Ph | S | methyl | methyl | b2 |
| 2-23-1070 | N—Ph | S | b1 | b1 | b2 |
| 2-23-1071 | N—Ph | S | b2 | b2 | b2 |
| 2-23-1072 | N—Ph | S | b3 | b3 | b2 |
| 2-23-1073 | N—Ph | S | H | H | b3 |
| 2-23-1074 | N—Ph | S | t-bu | t-bu | b3 |
| 2-23-1075 | N—Ph | S | methyl | methyl | b3 |
| 2-23-1076 | N—Ph | S | b1 | b1 | b3 |
| 2-23-1077 | N—Ph | S | b2 | b2 | b3 |
| 2-23-1078 | N—Ph | S | b3 | b3 | b3 |
| 2-23-1079 | N—Ph | S | H | H | b4 |
| 2-23-1080 | N—Ph | S | t-bu | t-bu | b4 |
| 2-23-1081 | N—Ph | S | methyl | methyl | b4 |
| 2-23-1082 | N—Ph | S | b1 | b1 | b4 |
| 2-23-1083 | N—Ph | S | b2 | b2 | b4 |
| 2-23-1084 | N—Ph | S | b3 | b3 | b4 |
| 2-23-1085 | N—Ph | S | H | H | b5 |
| 2-23-1086 | N—Ph | S | t-bu | t-bu | b5 |
| 2-23-1087 | N—Ph | S | methyl | methyl | b5 |
| 2-23-1088 | N—Ph | S | b1 | b1 | b5 |
| 2-23-1089 | N—Ph | S | b2 | b2 | b5 |
| 2-23-1090 | N—Ph | S | b3 | b3 | b5 |
| 2-23-1091 | N—Ph | S | H | H | b6 |
| 2-23-1092 | N—Ph | S | t-bu | t-bu | b6 |
| 2-23-1093 | N—Ph | S | methyl | methyl | b6 |
| 2-23-1094 | N—Ph | S | b1 | b1 | b6 |
| 2-23-1095 | N—Ph | S | b2 | b2 | b6 |
| 2-23-1096 | N—Ph | S | b3 | b3 | b6 |
| 2-23-1097 | N—Ph | S | H | H | b7 |
| 2-23-1098 | N—Ph | S | t-bu | t-bu | b7 |
| 2-23-1099 | N—Ph | S | methyl | methyl | b7 |
| 2-23-1100 | N—Ph | S | b1 | b1 | b7 |
| 2-23-1101 | N—Ph | S | b2 | b2 | b7 |
| 2-23-1102 | N—Ph | S | b3 | b3 | b7 |
| 2-23-1103 | N—Ph | S | H | H | b8 |
| 2-23-1104 | N—Ph | S | t-bu | t-bu | b8 |
| 2-23-1105 | N—Ph | S | methyl | methyl | b8 |
| 2-23-1106 | N—Ph | S | b1 | b1 | b8 |
| 2-23-1107 | N—Ph | S | b2 | b2 | b8 |
| 2-23-1108 | N—Ph | S | b3 | b3 | b8 |
| 2-23-1109 | N—Ph | S | H | H | b9 |
| 2-23-1110 | N—Ph | S | t-bu | t-bu | b9 |
| 2-23-1111 | N—Ph | S | methyl | methyl | b9 |
| 2-23-1112 | N—Ph | S | b1 | b1 | b9 |
| 2-23-1113 | N—Ph | S | b2 | b2 | b9 |
| 2-23-1114 | N—Ph | S | b3 | b3 | b9 |
| 2-23-1115 | N—Ph | S | H | H | b10 |
| 2-23-1116 | N—Ph | S | t-bu | t-bu | b10 |
| 2-23-1117 | N—Ph | S | methyl | methyl | b10 |
| 2-23-1118 | N—Ph | S | b1 | b1 | b10 |
| 2-23-1119 | N—Ph | S | b2 | b2 | b10 |
| 2-23-1120 | N—Ph | S | b3 | b3 | b10 |
| 2-23-1121 | N—Ph | S | H | H | H |
| 2-23-1122 | N—Ph | S | t-bu | t-bu | H |
| 2-23-1123 | N—Ph | S | methyl | methyl | H |
| 2-23-1124 | N—Ph | S | b1 | b1 | H |
| 2-23-1125 | N—Ph | S | b2 | b2 | H |
| 2-23-1126 | N—Ph | S | b3 | b3 | H |
| 2-23-1127 | N—Ph | S | H | H | H |
| 2-23-1128 | N—Ph | S | t-bu | t-bu | H |
| 2-23-1129 | N—Ph | S | methyl | methyl | H |
| 2-23-1130 | N—Ph | S | b1 | b1 | H |
| 2-23-1131 | N—Ph | S | b2 | b2 | H |
| 2-23-1132 | N—Ph | S | b3 | b3 | H |
| 2-23-1133 | N—Ph | S | H | H | H |
| 2-23-1134 | N—Ph | S | t-bu | t-bu | H |
| 2-23-1135 | N—Ph | S | methyl | methyl | H |
| 2-23-1136 | N—Ph | S | b1 | b1 | H |
| 2-23-1137 | N—Ph | S | b2 | b2 | H |
| 2-23-1138 | N—Ph | S | b3 | b3 | H |
| 2-23-1139 | N—Ph | S | H | H | H |
| 2-23-1140 | N—Ph | S | t-bu | t-bu | H |
| 2-23-1141 | N—Ph | S | methyl | methyl | H |
| 2-23-1142 | N—Ph | S | b1 | b1 | H |
| 2-23-1143 | N—Ph | S | b2 | b2 | H |
| 2-23-1144 | N—Ph | S | b3 | b3 | H |
| 2-23-1145 | N—Ph | S | H | H | H |
| 2-23-1146 | N—Ph | S | t-bu | t-bu | H |
| 2-23-1147 | N—Ph | S | methyl | methyl | H |
| 2-23-1148 | N—Ph | S | b1 | b1 | H |
| 2-23-1149 | N—Ph | S | b2 | b2 | H |
| 2-23-1150 | N—Ph | S | b3 | b3 | H |
| 2-23-1151 | N—Ph | S | H | H | H |
| 2-23-1152 | N—Ph | S | t-bu | t-bu | H |
| 2-23-1153 | N—Ph | S | methyl | methyl | H |
| 2-23-1154 | N—Ph | S | b1 | b1 | H |
| 2-23-1155 | N—Ph | S | b2 | b2 | H |
| 2-23-1156 | N—Ph | S | b3 | b3 | H |
| 2-23-1157 | N—Ph | S | H | H | H |
| 2-23-1158 | N—Ph | S | t-bu | t-bu | H |
| 2-23-1159 | N—Ph | S | methyl | methyl | H |
| 2-23-1160 | N—Ph | S | b1 | b1 | H |
| 2-23-1161 | N—Ph | S | b2 | b2 | H |
| 2-23-1162 | N—Ph | S | b3 | b3 | H |
| 2-23-1163 | N—Ph | S | H | H | H |
| 2-23-1164 | N—Ph | S | t-bu | t-bu | H |
| 2-23-1165 | N—Ph | S | methyl | methyl | H |
| 2-23-1166 | N—Ph | S | b1 | b1 | H |
| 2-23-1167 | N—Ph | S | b2 | b2 | H |
| 2-23-1168 | N—Ph | S | b3 | b3 | H |
| 2-23-1169 | N—Ph | S | H | H | H |
| 2-23-1170 | N—Ph | S | t-bu | t-bu | H |
| 2-23-1171 | N—Ph | S | methyl | methyl | H |
| 2-23-1172 | N—Ph | S | b1 | b1 | H |
| 2-23-1173 | N—Ph | S | b2 | b2 | H |
| 2-23-1174 | N—Ph | S | b3 | b3 | H |
| 2-23-1175 | N—Ph | S | H | H | H |
| 2-23-1176 | N—Ph | S | t-bu | t-bu | H |
| 2-23-1177 | N—Ph | S | methyl | methyl | H |
| 2-23-1178 | N—Ph | S | b1 | b1 | H |
| 2-23-1179 | N—Ph | S | b2 | b2 | H |
| 2-23-1180 | N—Ph | S | b3 | b3 | H |
| 2-23-1181 | N—Ph | S | H | H | H |
| 2-23-1182 | N—Ph | S | t-bu | t-bu | H |
| 2-23-1183 | N—Ph | S | methyl | methyl | H |
| 2-23-1184 | N—Ph | S | b1 | b1 | H |
| 2-23-1185 | N—Ph | S | b2 | b2 | H |
| 2-23-1186 | N—Ph | S | b3 | b3 | H |
| 2-23-1187 | N—Ph | S | H | H | H |
| 2-23-1188 | N—Ph | S | t-bu | t-bu | H |
| 2-23-1189 | N—Ph | S | methyl | methyl | H |
| 2-23-1190 | N—Ph | S | b1 | b1 | H |
| 2-23-1191 | N—Ph | S | b2 | b2 | H |
| 2-23-1192 | N—Ph | S | b3 | b3 | H |
| 2-23-1193 | N—Ph | S | H | H | H |
| 2-23-1194 | N—Ph | S | t-bu | t-bu | H |
| 2-23-1195 | N—Ph | S | methyl | methyl | H |
| 2-23-1196 | N—Ph | S | b1 | b1 | H |
| 2-23-1197 | N—Ph | S | b2 | b2 | H |
| 2-23-1198 | N—Ph | S | b3 | b3 | H |
| 2-23-1199 | N—Ph | S | H | H | H |
| 2-23-1200 | N—Ph | S | t-bu | t-bu | H |
| 2-23-1201 | N—Ph | S | methyl | methyl | H |
| 2-23-1202 | N—Ph | S | b1 | b1 | H |
| 2-23-1203 | N—Ph | S | b2 | b2 | H |
| 2-23-1204 | N—Ph | S | b3 | b3 | H |
| 2-23-1205 | N—Ph | S | H | H | H |
| 2-23-1206 | N—Ph | S | t-bu | t-bu | H |
| 2-23-1207 | N—Ph | S | methyl | methyl | H |
| 2-23-1208 | N—Ph | S | b1 | b1 | H |
| 2-23-1209 | N—Ph | S | b2 | b2 | H |
| 2-23-1210 | N—Ph | S | b3 | b3 | H |
| 2-23-1211 | N—Ph | S | H | H | H |
| 2-23-1212 | N—Ph | S | t-bu | t-bu | H |
| 2-23-1213 | N—Ph | S | methyl | methyl | H |
| 2-23-1214 | N—Ph | S | b1 | b1 | H |
| 2-23-1215 | N—Ph | S | b2 | b2 | H |
| 2-23-1216 | N—Ph | S | b3 | b3 | H |
| 2-23-1217 | N—Ph | S | H | H | a1 |
| 2-23-1218 | N—Ph | S | t-bu | t-bu | a1 |
| 2-23-1219 | N—Ph | S | methyl | methyl | a1 |
| 2-23-1220 | N—Ph | S | b1 | b1 | a1 |
| 2-23-1221 | N—Ph | S | b2 | b2 | a1 |
| 2-23-1222 | N—Ph | S | b3 | b3 | a1 |
| 2-23-1223 | N—Ph | S | H | H | a2 |
| 2-23-1224 | N—Ph | S | t-bu | t-bu | a2 |
| 2-23-1225 | N—Ph | S | methyl | methyl | a2 |
| 2-23-1226 | N—Ph | S | b1 | b1 | a2 |
| 2-23-1227 | N—Ph | S | b2 | b2 | a2 |
| 2-23-1228 | N—Ph | S | b3 | b3 | a2 |
| 2-23-1229 | N—Ph | S | H | H | a3 |
| 2-23-1230 | N—Ph | S | t-bu | t-bu | a3 |
| 2-23-1231 | N—Ph | S | methyl | methyl | a3 |
| 2-23-1232 | N—Ph | S | b1 | b1 | a3 |
| 2-23-1233 | N—Ph | S | b2 | b2 | a3 |
| 2-23-1234 | N—Ph | S | b3 | b3 | a3 |
| 2-23-1235 | N—Ph | S | H | H | b1 |
| 2-23-1236 | N—Ph | S | t-bu | t-bu | b1 |
| 2-23-1237 | N—Ph | S | methyl | methyl | b1 |
| 2-23-1238 | N—Ph | S | b1 | b1 | b1 |
| 2-23-1239 | N—Ph | S | b2 | b2 | b1 |
| 2-23-1240 | N—Ph | S | b3 | b3 | b1 |
| 2-23-1241 | N—Ph | S | H | H | b2 |
| 2-23-1242 | N—Ph | S | t-bu | t-bu | b2 |
| 2-23-1243 | N—Ph | S | methyl | methyl | b2 |
| 2-23-1244 | N—Ph | S | b1 | b1 | b2 |
| 2-23-1245 | N—Ph | S | b2 | b2 | b2 |
| 2-23-1246 | N—Ph | S | b3 | b3 | b2 |
| 2-23-1247 | N—Ph | S | H | H | b3 |
| 2-23-1248 | N—Ph | S | t-bu | t-bu | b3 |
| 2-23-1249 | N—Ph | S | methyl | methyl | b3 |
| 2-23-1250 | N—Ph | S | b1 | b1 | b3 |
| 2-23-1251 | N—Ph | S | b2 | b2 | b3 |
| 2-23-1252 | N—Ph | S | b3 | b3 | b3 |
| 2-23-1253 | N—Ph | S | H | H | b5 |
| 2-23-1254 | N—Ph | S | t-bu | t-bu | b5 |
| 2-23-1255 | N—Ph | S | methyl | methyl | b5 |
| 2-23-1256 | N—Ph | S | b1 | b1 | b5 |
| 2-23-1257 | N—Ph | S | b2 | b2 | b5 |
| 2-23-1258 | N—Ph | S | b3 | b3 | b5 |
| 2-23-1259 | N—Ph | S | H | H | b6 |
| 2-23-1260 | N—Ph | S | t-bu | t-bu | b6 |
| 2-23-1261 | N—Ph | S | methyl | methyl | b6 |
| 2-23-1262 | N—Ph | S | b1 | b1 | b6 |
| 2-23-1263 | N—Ph | S | b2 | b2 | b6 |
| 2-23-1264 | N—Ph | S | b3 | b3 | b6 |
| 2-23-1265 | N—Ph | S | H | H | b7 |
| 2-23-1266 | N—Ph | S | t-bu | t-bu | b7 |
| 2-23-1267 | N—Ph | S | methyl | methyl | b7 |
| 2-23-1268 | N—Ph | S | b1 | b1 | b7 |
| 2-23-1269 | N—Ph | S | b2 | b2 | b7 |
| 2-23-1270 | N—Ph | S | b3 | b3 | b7 |

Group 2-24
The compounds are represented by the formula 2-24 below, wherein X₁ is B, and Y1, Y2, Y3, R1b, R2b, R3a are each shown in the table 24 below, 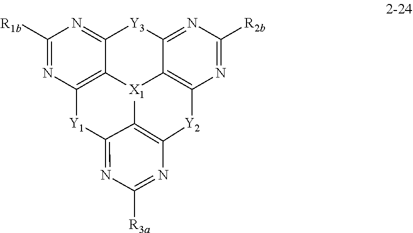
TABLE 24

| Compound | | | | |
|---|---|---|---|---|
| No. | Y1/Y2/Y3 | R1b | R2b | R3a |
| 2-24-001 | N-Ph | H | H | H |
| 2-24-002 | N-Ph | t-bu | t-bu | t-bu |
| 2-24-003 | N-Ph | methyl | methyl | methyl |
| 2-24-004 | N-Ph | b1 | b1 | b1 |
| 2-24-005 | N-Ph | b2 | b2 | b2 |
| 2-24-006 | N-Ph | b3 | b3 | b3 |
| 2-24-007 | N-Ph | b4 | b4 | b4 |
| 2-24-008 | N-Ph | b5 | b5 | b5 |
| 2-24-009 | N-Ph | b6 | b6 | b6 |
| 2-24-010 | N-Ph | b7 | b7 | b7 |
| 2-24-011 | N-Ph | b8 | b8 | b8 |
| 2-24-012 | N-Ph | b9 | b9 | b9 |
| 2-24-013 | N-Ph | b10 | b10 | b10 |
| 2-24-014 | N-Ph | a1 | a1 | a1 |
| 2-24-015 | N-Ph | a2 | a2 | a2 |
| 2-24-016 | N-Ph | a3 | a3 | a3 |
| 2-24-017 | N-Ph | a4 | a4 | a4 |
| 2-24-018 | N-Ph | a5 | a5 | a5 |
| 2-24-019 | N-Ph | a6 | a6 | a6 |
| 2-24-020 | N-tolyl | H | H | H |
| 2-24-021 | N-tolyl | t-bu | t-bu | t-bu |
| 2-24-022 | N-tolyl | methyl | methyl | methyl |
| 2-24-023 | N-tolyl | b1 | b1 | b1 |
| 2-24-024 | N-tolyl | b2 | b2 | b2 |
| 2-24-025 | N-tolyl | b3 | b3 | b3 |
| 2-24-026 | N-tolyl | b4 | b4 | b4 |
| 2-24-027 | N-tolyl | b5 | b5 | b5 |
| 2-24-028 | N-tolyl | b6 | b6 | b6 |
| 2-24-029 | N-tolyl | b7 | b7 | b7 |
| 2-24-030 | N-tolyl | b8 | b8 | b8 |
| 2-24-031 | N-tolyl | b9 | b9 | b9 |
| 2-24-032 | N-tolyl | b10 | b10 | b10 |
| 2-24-033 | N-tolyl | a1 | a1 | a1 |
| 2-24-034 | N-tolyl | a2 | a2 | a2 |
| 2-24-035 | N-tolyl | a3 | a3 | a3 |
| 2-24-036 | N-tolyl | a4 | a4 | a4 |
| 2-24-037 | N-tolyl | a5 | a5 | a5 |
| 2-24-038 | N-tolyl | a6 | a6 | a6 |
| 2-24-039 | N-Biph | H | H | H |
| 2-24-040 | N-Biph | t-bu | t-bu | t-bu |
| 2-24-041 | N-Biph | methyl | methyl | methyl |
| 2-24-042 | N-Biph | b1 | b1 | b1 |
| 2-24-043 | N-Biph | b2 | b2 | b2 |
| 2-24-044 | N-Biph | b3 | b3 | b3 |
| 2-24-045 | N-Biph | b4 | b4 | b4 |
| 2-24-046 | N-Biph | b5 | b5 | b5 |
| 2-24-047 | N-Biph | b6 | b6 | b6 |
| 2-24-048 | N-Biph | b7 | b7 | b7 |
| 2-24-049 | N-Biph | b8 | b8 | b8 |
| 2-24-050 | N-Biph | b9 | b9 | b9 |
| 2-24-051 | N-Biph | b10 | b10 | b10 |
| 2-24-052 | N-Biph | a1 | a1 | a1 |
| 2-24-053 | N-Biph | a2 | a2 | a2 |
| 2-24-054 | N-Biph | a3 | a3 | a3 |
| 2-24-055 | N-Biph | a4 | a4 | a4 |
| 2-24-056 | N-Biph | a5 | a5 | a5 |
| 2-24-057 | N-Biph | a6 | a6 | a6 |
| 2-24-058 | Se | H | H | H |
| 2-24-059 | Se | t-bu | t-bu | t-bu |
| 2-24-060 | Se | methyl | methyl | methyl |
| 2-24-061 | Se | b1 | b1 | b1 |
| 2-24-062 | Se | b2 | b2 | b2 |
| 2-24-063 | Se | b3 | b3 | b3 |
| 2-24-064 | Se | b4 | b4 | b4 |
| 2-24-065 | Se | b5 | b5 | b5 |
| 2-24-066 | Se | b6 | b6 | b6 |
| 2-24-067 | Se | b7 | b7 | b7 |
| 2-24-068 | Se | b8 | b8 | b8 |
| 2-24-069 | Se | b9 | b9 | b9 |
| 2-24-070 | Se | b10 | b10 | b10 |
| 2-24-071 | Se | a1 | a1 | a1 |
| 2-24-072 | Se | a2 | a2 | a2 |
| 2-24-073 | Se | a3 | a3 | a3 |
| 2-24-074 | Se | a4 | a4 | a4 |
| 2-24-075 | Se | a5 | a5 | a5 |
| 2-24-076 | Se | a6 | a6 | a6 |
| 2-24-077 | PO | H | H | H |
| 2-24-078 | PO | t-bu | t-bu | t-bu |
| 2-24-079 | PO | methyl | methyl | methyl |
| 2-24-080 | PO | b1 | b1 | b1 |
| 2-24-081 | PO | b2 | b2 | b2 |
| 2-24-082 | PO | b3 | b3 | b3 |
| 2-24-083 | PO | b4 | b4 | b4 |
| 2-24-084 | PO | b5 | b5 | b5 |
| 2-24-085 | PO | b6 | b6 | b6 |
| 2-24-086 | PO | b7 | b7 | b7 |
| 2-24-087 | PO | b8 | b8 | b8 |
| 2-24-088 | PO | b9 | b9 | b9 |
| 2-24-089 | PO | b10 | b10 | b10 |
| 2-24-090 | PO | a1 | a1 | a1 |
| 2-24-091 | PO | a2 | a2 | a2 |
| 2-24-092 | PO | a3 | a3 | a3 |
| 2-24-093 | PO | a4 | a4 | a4 |
| 2-24-094 | PO | a5 | a5 | a5 |
| 2-24-095 | PO | a6 | a6 | a6 |

Group 2-25
The compounds are represented by the formula 2-25 below, wherein X₁ is B, and R1 b, R1c, R1d, R2b, R2c, R2d, R3a, R10a, R10b, R10c, R10d, R10e, R10f, R10g, R10h are each shown in the table 25 below, 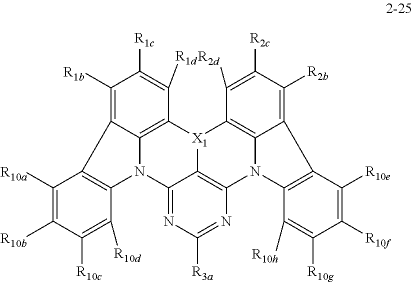
TABLE 25

| Compound | R1c/R1d/R10b/ | R2c/R2d/ | | R1b/R2b/R10a/R10d/ |
|---|---|---|---|---|
| No. | R10c | R10f/R10g | R3a | R10e/R10h |
| 2-25-001 | H | H | H | H |
| 2-25-002 | t-bu | H | H | H |
| 2-25-003 | methyl | H | H | H |
| 2-25-004 | b1 | H | H | H |
| 2-25-005 | b2 | H | H | H |
| 2-25-006 | b3 | H | H | H |
| 2-25-007 | b4 | H | H | H |
| 2-25-008 | b5 | H | H | H |
| 2-25-009 | b6 | H | H | H |
| 2-25-010 | b7 | H | H | H |
| 2-25-011 | b8 | H | H | H |
| 2-25-012 | b9 | H | H | H |
| 2-25-013 | b10 | H | H | H |
| 2-25-014 | a1 | H | H | H |
| 2-25-015 | a2 | H | H | H |
| 2-25-016 | a3 | H | H | H |
| 2-25-017 | a4 | H | H | H |
| 2-25-018 | a5 | H | H | H |
| 2-25-019 | a6 | H | H | H |
| 2-25-020 | H | H | H | H |
| 2-25-021 | H | t-bu | H | H |
| 2-25-022 | H | methyl | H | H |
| 2-25-023 | H | b1 | H | H |
| 2-25-024 | H | b2 | H | H |
| 2-25-025 | H | b3 | H | H |
| 2-25-026 | H | b4 | H | H |
| 2-25-027 | H | b5 | H | H |
| 2-25-028 | H | b6 | H | H |
| 2-25-029 | H | b7 | H | H |
| 2-25-030 | H | b8 | H | H |
| 2-25-031 | H | b9 | H | H |
| 2-25-032 | H | b10 | H | H |
| 2-25-033 | H | a1 | H | H |
| 2-25-034 | H | a2 | H | H |
| 2-25-035 | H | a3 | H | H |
| 2-25-036 | H | a4 | H | H |
| 2-25-037 | H | a5 | H | H |
| 2-25-038 | H | a6 | H | H |
| 2-25-039 | H | H | H | H |
| 2-25-040 | H | H | t-bu | H |
| 2-25-041 | H | H | methyl | H |
| 2-25-042 | H | H | b1 | H |
| 2-25-043 | H | H | b2 | H |
| 2-25-044 | H | H | b3 | H |
| 2-25-045 | H | H | b4 | H |
| 2-25-046 | H | H | b5 | H |
| 2-25-047 | H | H | b6 | H |
| 2-25-048 | H | H | b7 | H |
| 2-25-049 | H | H | b8 | H |
| 2-25-050 | H | H | b9 | H |
| 2-25-051 | H | H | b10 | H |
| 2-25-052 | H | H | a1 | H |
| 2-25-053 | H | H | a2 | H |
| 2-25-054 | H | H | a3 | H |
| 2-25-055 | H | H | a4 | H |
| 2-25-056 | H | H | a5 | H |
| 2-25-057 | H | H | a6 | H |
| 2-25-058 | H | H | t-bu | H |
| 2-25-059 | t-bu | H | methyl | H |
| 2-25-060 | methyl | H | b1 | H |
| 2-25-061 | b1 | H | b2 | H |
| 2-25-062 | b2 | H | b3 | H |
| 2-25-063 | b3 | H | H | H |
| 2-25-064 | b4 | H | t-bu | H |
| 2-25-065 | b5 | H | methyl | H |
| 2-25-066 | b6 | H | b1 | H |
| 2-25-067 | b7 | H | b2 | H |
| 2-25-068 | b8 | H | b3 | H |
| 2-25-069 | b9 | H | H | H |
| 2-25-070 | b10 | H | t-bu | H |
| 2-25-071 | a1 | H | methyl | H |
| 2-25-072 | a2 | H | b1 | H |
| 2-25-073 | a3 | H | b2 | H |
| 2-25-074 | a4 | H | b3 | H |
| 2-25-075 | a5 | H | H | H |
| 2-25-076 | a6 | H | t-bu | H |

In the tables 1 to 25 regarding the groups 2-1 to 2-25 above, “t-bu” indicates “tert-butyl group” and “N-Ph”, “N-tolyl”, “N-Biph”, “a1”, “a2”, “a3, “a4”, “a5”, “a6”, “b1”, “b2”, “b3”, “b4”, “b5”, “b6”, “b7”, “b8”, “b9”, and “b10” indicates as shown below, and Y indicates Y₁, Y₂ or Y₃:

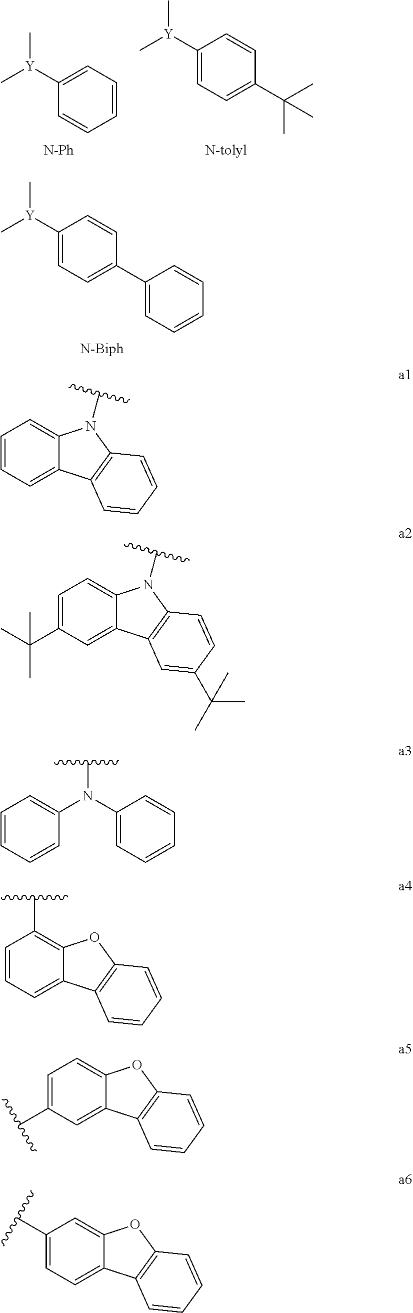

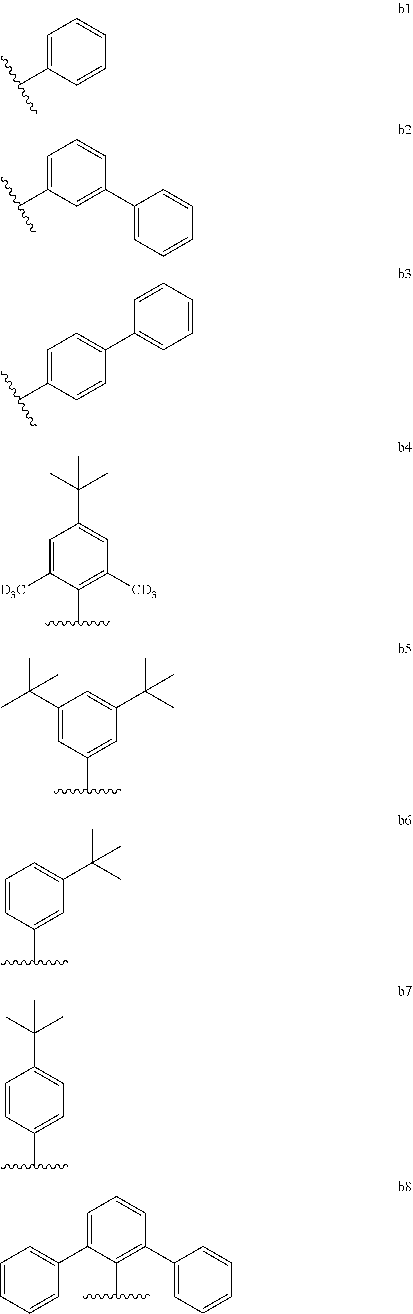

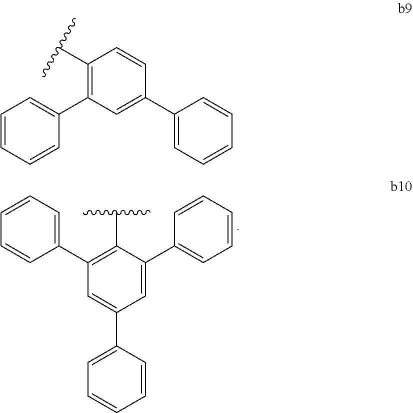

15. An organic light-emitting device comprising:
a first electrode,
a second electrode,
an organic layer located between the first electrode and the second electrode, and comprising an emission layer, and
wherein the organic layer comprises the heterocyclic compound of claim 1.

16. The organic light-emitting device of claim 15, wherein
the emission layer comprises the heterocyclic compound of claim 1.

17. The organic light-emitting device of claim 16, wherein
the heterocyclic compound is a fluorescent emitter.

18. The organic light-emitting device of claim 16, wherein
the emission layer further comprises a sensitizer which satisfies Equation 1:
ΔE_{ST}≤0.3 eV Equation 1
where ΔE_{ST} refers to the energy difference between a lowest excited singlet (S₁) and a lowest excited triplet (T₁).

19. The organic light-emitting device of claim 18, wherein
the sensitizer and the heterocyclic compound satisfy Conditions 1 and 2:
T_{decay}(HC)<T_{decay}(S) Condition 1
T_{decay}(HC)<1.5 μs Condition 2
wherein, in Conditions 1 and 2,
T_{decay} (HC) is a decay time of the heterocyclic compound, and
T_{decay}(S) is a decay time of the sensitizer.

20. The organic light-emitting device of claim 15, wherein
the emission layer further includes a light-emitting dopant and a host, and
an amount of the host is greater than a total amount of the heterocyclic compound and the light-emitting dopant.
